(19) United States
(12) Patent Application Publication
(10) Pub. No.: US 2025/0206746 A1
(43) Pub. Date: Jun. 26, 2025

(54) 4-ALKOXYPYRROLO[2,1-F][1,2,4]TRIAZINES AND PREPARATION AND USES THEREOF

(71) Applicant: BioSplice Therapeutics, Inc., San Diego, CA (US)

(72) Inventors: Gopi Kumar Mittapalli, San Diego, CA (US); Chi Ching Mak, San Diego, CA (US); Ramkrishna Reddy Vakiti, San Diego, CA (US); Brian Joseph Hofilena, San Diego, CA (US); Brian Walter Eastman, San Diego, CA (US); Lewis Daniel Turner, San Diego, CA (US)

(21) Appl. No.: 18/990,148

(22) Filed: Dec. 20, 2024

Related U.S. Application Data
(60) Provisional application No. 63/614,120, filed on Dec. 22, 2023.

(51) Int. Cl. C07D 487/04; A61K 31/53; A61K 31/5377; C07D 519/00

(52) U.S. Cl. CPC C07D 487/04; A61K 31/53; A61K 31/5377; C07D 519/00

(57) ABSTRACT

4-Alkoxypyrrolo[2,1-f][1,2,4]triazine compounds for treating various diseases and pathologies are disclosed. More particularly, the present disclosure concerns the use of 4-alkoxypyrrolo[2,1-f][1,2,4]triazine compounds or analogs thereof, in the treatment of disorders characterized by overexpression of DYRK1A (e.g., cancer, Down syndrome, Alzheimer's disease, diabetes, viral infections, and osteoarthritis).

### RELATED APPLICATIONS

This application claims the benefit of U.S. Provisional Application No. 63/614,120, filed Dec. 22, 2023, which is incorporated herein by reference in its entirety.

### BACKGROUND

### Technical Field

This disclosure relates to inhibitors of dual-specificity tyrosine phosphorylation-regulated 1A kinase, and compositions comprising the same. More particularly, it concerns the use of a 4-alkoxypyrrolo[2,1-f][1,2,4]triazine compounds or salts or analogs thereof, in the treatment of disorders characterized by the abnormal expression and/or activity of DYRK1A (e.g., cancer, Down syndrome, Alzheimer's disease, diabetes, viral infections, and osteoarthritis).

### Background

Dual-specificity tyrosine phosphorylation-regulated kinases (DYRK1A, 1B, 2-4) comprise a family of protein kinases within the CMGC group of the eukaryotic kinome. These protein kinases are involved in multiple cellular functions, including intracellular signaling, mRNA splicing, chromatin transcription, DNA damage repair, cell survival, cell cycle control, differentiation, homocysteine/methionine/folate regulation, body temperature regulation, endocytosis, neuronal development, synaptic plasticity, etc. Abnormal expression and/or activity of some of these kinases, DYRK1A in particular, is seen in many human nervous system diseases, such as cognitive deficits associated with Down syndrome, Alzheimer's disease, and related diseases, tauopathies, dementia, Pick's disease, Parkinson's disease, and other neurodegenerative diseases, Phelan-McDermid syndrome, autism, and CDKL5 deficiency disorder. DYRKs are also involved in diabetes, abnormal folate/methionine metabolism, osteoarthritis, several solid cancers (glioblastoma, breast, and pancreatic cancers) and leukemias (acute lymphoblastic leukemia, acute megakaryoblastic leukemia), viral infections (influenza, HIV-1, HCMV, HCV, CMV, HPV), as well as infections caused by unicellular parasites (Leishmania, Trypanosoma, Plasmodium) (International Journal of Molecular Sciences (2021), 22(11), 6047). DYRK1A has also been identified as a critical stabilizer of EGFR (Cell Death & Disease (2019), 10, 282) which is a crucial factor contributing to the keratinization, cell hyperproliferation, abnormal differentiation and inflammatory infiltration during the progress of psoriasis.

### SUMMARY

The present disclosure provides methods and reagents, the method involving contacting a cell with an agent, such as a 4-alkoxypyrrolo[2,1-f][1,2,4]triazine compound, in a sufficient amount to antagonize DYRK1A activity, e.g., reduce the proliferation of head and neck squamous cell carcinoma, luminal/HER2 breast cancer, (Cell (2016), 164(1-2), 293-309) or pancreatic adenocarcinoma, as well as impair the self-renewal capacity of glioblastoma and compromise ovarian cancer spheroid cell viability (Molecular Cancer Research (2017), 15(4), 371-381).

The present disclosure also provides methods and reagents, the method involving contacting a cell with an agent, such as a 4-alkoxypyrrolo[2,1-f][1,2,4]triazine compound, in a sufficient amount to antagonize DYRK1A activity, e.g., i) to normalize prenatal and early postnatal brain development; ii) to improve cognitive function in youth and adulthood; and/or iii) to attenuate Alzheimer's-type neurodegeneration.

Some embodiments disclosed herein include DYRK1A inhibitors containing a 4-alkoxypyrrolo[2,1-f][1,2,4]triazine core. Other embodiments disclosed herein include pharmaceutical compositions and methods of treatment using these compounds.

One embodiment disclosed herein includes a compound having the structure of Formula I.

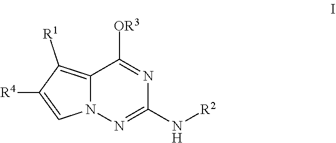

or a pharmaceutically acceptable salt thereof,
wherein,
R¹ is a 9-10 membered heteroaryl optionally substituted with 1-10 R⁵;
R² is selected from the group consisting of —(C₁₋₅ alkylene)CN, -heterocyclyl optionally substituted with 1-10 R⁶, —(C₁₋₂ alkylene)C(═O)heterocyclyl, and -carbocyclyl optionally substituted with 1-12 R⁷, wherein the —(C₁₋₂ alkylene) or (C₁₋₅ alkylene) is optionally substituted with 1-2 halide and/or 1-2 unsubstituted —(C₁₋₃ alkyl);
R³ is unsubstituted —(C₁₋₃ alkyl);
R⁴ is selected from the group consisting of H and halide;
each R⁵ is independently selected from the group consisting of halide, unsubstituted —(C₁₋₉ alkyl), unsubstituted —(C₁₋₉ haloalkyl), —(C₁₋₄ alkylene)OH, -carbocyclyl optionally substituted with 1-12 halides, —C(═O)N(R⁸)₂, wherein the —(C₁₋₄ alkylene) is optionally substituted with 1-4 halide and/or 1-2 unsubstituted —(C₁₋₃ alkyl);
each R⁶ is independently selected from the group consisting of halide, unsubstituted —(C₁₋₉ alkyl), unsubstituted-heterocyclyl, —(C₁₋₃ alkylene)OR⁹, —SO₂Me, and —(C₁₋₃ alkylene)ₚC(═O)R¹⁰, wherein each —(C₁₋₃ alkylene) is, independently, optionally substituted with 1-2 halide and/or 1-2 unsubstituted —(C₁₋₃ alkyl);
alternatively, two R⁶ attached to the same carbon atom are taken together to form a carbonyl group;
each R⁷ is independently selected from the group consisting of halide, unsubstituted —(C₁₋₉ alkyl), —OH, and —NHC(═O)R¹;
each R⁸ is independently selected from the group consisting of H, unsubstituted —(C₁₋₉ alkyl), and unsubstituted —(C₁₋₉ haloalkyl);
each R⁹ is independently selected from the group consisting of H and unsubstituted —(C₁₋₉ alkyl);
each R¹⁰ is independently selected from the group consisting of unsubstituted —(C₁₋₉ alkyl), —(C₁₋₂ alkylene)ₚN(R¹²)₂, —(C₁₋₂ alkylene)OR⁹, —(C₁₋₂ alkylene)ₚheterocyclyl, and unsubstituted-carbocyclyl, wherein each —(C₁₋₂ alkylene) is, independently, optionally substituted with 1-2 halide and/or 1-2 unsubstituted —(C₁₋₃ alkyl);
each R¹¹ is independently selected from the group consisting of unsubstituted —(C₁₋₉ alkyl), —(C₁₋₂ alkylene)ₚOR⁹, and -carbocyclyl optionally substituted with 1-12 halides, wherein the —(C₁₋₂ alkylene) is optionally substituted with 1-2 halide and/or 1-2 unsubstituted —(C₁₋₃ alkyl);
each R¹² is independently selected from the group consisting of H and unsubstituted —(C₁₋₉ alkyl);
each p is independently 0 or 1; and
wherein each H atom is optionally, independently replaced by ²H (D) (deuterium).

In another embodiment disclosed herein includes a compound having the structure of Formula I:

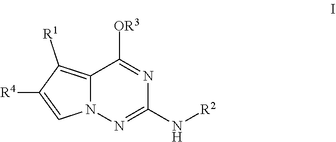

or a pharmaceutically acceptable salt thereof,
wherein,
R¹ is a 9-10 membered heteroaryl optionally substituted with 1-10 R⁵;
R² is selected from the group consisting of -heterocyclyl optionally substituted with 1-10 R⁶ and —(C₁₋₂ alkylene)C(═O)heterocyclyl, wherein the —(C₁₋₂ alkylene) is optionally substituted with 1-2 halide and/or 1-2 unsubstituted —(C₁₋₃ alkyl);
R³ is unsubstituted —(C₁₋₃ alkyl);
R⁴ is selected from the group consisting of H and halide;
each R⁵ is independently selected from the group consisting of halide, unsubstituted —(C₁₋₉ alkyl), unsubstituted —(C₁₋₉ haloalkyl), —(C₁₋₄ alkylene)OH, -carbocyclyl optionally substituted with 1-12 halides, —C(═O)N(R)₂, wherein the —(C₁₋₄ alkylene) is optionally substituted with 1-4 halide and/or 1-2 unsubstituted —(C₁₋₃ alkyl);
each R⁶ is —(C₁₋₃ alkylene)ₚC(═O)R¹⁰, wherein the —(C₁₋₃ alkylene) is optionally substituted with 1-2 halide and/or 1-2 unsubstituted —(C₁₋₃ alkyl);
each R⁸ is independently selected from the group consisting of H, unsubstituted —(C₁₋₉ alkyl), and unsubstituted —(C₁₋₉ haloalkyl);
each R⁹ is independently selected from the group consisting of H and unsubstituted —(C₁₋₉ alkyl);
each R¹⁰ is independently selected from the group consisting of —(C₁₋₂ alkylene)ₚN(R¹²)₂, —(C₁₋₂ alkylene)OR⁹, and —(C₁₋₂ alkylene)ₚheterocyclyl, wherein each —(C₁₋₂ alkylene) is, independently, optionally substituted with 1-2 halide and/or 1-2 unsubstituted —(C₁₋₃ alkyl);
each R¹² is independently selected from the group consisting of H and unsubstituted —(C₁₋₉ alkyl);
each p is independently 0 or 1; and
wherein each H atom is optionally, independently replaced by ²H (D) (deuterium).

Some embodiments include stereoisomers and pharmaceutically acceptable salts of a compound of Formula I. Some embodiments include pharmaceutically acceptable salts of a compound of Formula I.

Some embodiments include pro-drugs of a compound of Formula I.

Some embodiments of the present disclosure include pharmaceutical compositions comprising a compound of Formula I and a pharmaceutically acceptable carrier, diluent, or excipient.

Other embodiments disclosed herein include methods of inhibiting DYRK1A by administering to a patient affected by a disorder or disease in which DYRK1A overexpression is implicated, such as Alzheimer's disease, amyotrophic lateral sclerosis, CDKL5 deficiency disorder, Down syndrome, frontotemporal dementia with parkinsonism-17 (FTDP-17), Lewy body dementia, Parkinson's disease, Pick's disease, and additional diseases with pronounced neurodegeneration such as autism, dementia, epilepsy, Huntington's disease, multiple sclerosis; diseases and disorders associated with acquired brain injury such as chronic traumatic encephalopathy, traumatic brain injury, tumor, and stroke.

Inhibitors of DYRK1A can also be used to treat tauopathies. Tauopathies are neurodegenerative disorders characterized by the deposition of abnormal tau protein in the brain. The spectrum of tau pathologies expands beyond the traditionally discussed disease forms like Pick's disease, progressive supranuclear palsy, corticobasal degeneration, and argyrophilic grain disease. Emerging entities and pathologies include globular glial tauopathies, primary age-related tauopathy, which includes neurofibrillary tangle dementia, chronic traumatic encephalopathy (CTE), frontotemporal lobar degeneration with tau inclusions (FTLD-tau), and aging-related tau astrogliopathy. Clinical symptoms include frontotemporal dementia, corticobasal syndrome, Richardson syndrome, parkinsonism, pure akinesia with gait freezing and, rarely, motor neuron symptoms or cerebellar ataxia (Handbook of Clinical Neurology (2018), 145, 355-368 and Aging Cell (2019), 18(5), e13000).

Inhibitors of DYRK1A can also be used to treat disorders associated with abnormal folate/methionine metabolism.

Non-limiting examples of diseases which can be treated with the compounds and compositions provided herein include a variety of cancers, diabetes, psoriasis, knee osteoarthritis, tendinopathy, human immunodeficiency virus type 1 (HIV-1), human cytomegalovirus (HCMV), hepatitis C virus (HCV), and herpes simplex virus 1 (HSV-1).

Some embodiments of the present disclosure include methods to prepare compounds of Formula I.

It is to be understood that both the foregoing general description and the following detailed description are exemplary and explanatory only and are not restrictive of the disclosure, as claimed.

### DETAILED DESCRIPTION

Provided herein are compositions and methods for inhibiting DYRK1A.

Some embodiments provided herein relate to a method for treating a disease including, but not limited to, neurological diseases or disorders, cancers, cognitive deficits, knee osteoarthritis, tendinopathy, viral infections, unicellular parasite infections, and motor deficits.

In some embodiments, non-limiting examples of a neurological disease or disorder which can be treated with the compounds and compositions provided herein include, but are not limited to, Alzheimer's disease, amyotrophic lateral sclerosis, Down syndrome, frontotemporal dementia with parkinsonism-17 (FTDP-17), Lewy body dementia, Parkinson's disease, Pick's disease tauopathies, and additional diseases with pronounced neurodegeneration such as autism, dementia, epilepsy, Huntington's disease, multiple sclerosis; diseases and disorders associated with acquired brain injury such as chronic traumatic encephalopathy, traumatic brain injury, tumor, and stroke.

In some embodiments, non-limiting examples of cancers which can be treated with the compounds and compositions provided herein include solid cancers (e.g., glioblastoma, ovarian, breast, and pancreatic cancers) and leukemias (e.g., acute lymphoblastic leukemia, acute megakaryoblastic leukemia, and chronic myeloid leukemia).

In some embodiments, pharmaceutical compositions are provided that are effective for treatment of a disease of an animal, e.g., a mammal, caused by DYRK1A overexpression. The composition includes a pharmaceutically acceptable carrier and a compound as described herein.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used herein, “alkyl” means a branched, or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl and neo-pentyl. Alkyl groups can either be unsubstituted or substituted with one or more substituents. In some embodiments, alkyl groups include 1 to 9 carbon atoms (for example, 1 to 6 carbon atoms, 1 to 4 carbon atoms, or 1 to 2 carbon atoms).

As used herein, “alkenyl” means a straight or branched chain chemical group containing only carbon and hydrogen and containing at least one carbon-carbon double bond, such as ethenyl, 1-propenyl, 2-propenyl, 2-methyl-1-propenyl, 1-butenyl, 2-butenyl, and the like. In various embodiments, alkenyl groups can either be unsubstituted or substituted with one or more substituents. Typically, alkenyl groups will comprise 2 to 9 carbon atoms (for example, 2 to 6 carbon atoms, 2 to 4 carbon atoms, or 2 carbon atoms).

As used herein, “alkynyl” means a straight or branched chain chemical group containing only carbon and hydrogen and containing at least one carbon-carbon triple bond, such as ethynyl, 1-propynyl, 1-butynyl, 2-butynyl, and the like. In various embodiments, alkynyl groups can either be unsubstituted or substituted with one or more substituents. Typically, alkynyl groups will comprise 2 to 9 carbon atoms (for example, 2 to 6 carbon atoms, 2 to 4 carbon atoms, or 2 carbon atoms).

As used herein, “alkylene” means a bivalent branched or straight chain chemical group containing only carbon and hydrogen, such as methylene, ethylene, n-propylene, iso-propylene, n-butylene, iso-butylene, sec-butylene, tert-butylene, n-pentylene, iso-pentylene, sec-pentylene and neo-pentylene. Alkylene groups can either be unsubstituted or substituted with one or more substituents. In some embodiments, alkylene groups include 1 to 9 carbon atoms (for example, 1 to 6 carbon atoms, 1 to 4 carbon atoms, or 1 to 2 carbon atoms).

As used herein, “alkenylene” means a bivalent branched or straight chain chemical group containing only carbon and hydrogen and containing at least one carbon-carbon double bond, such as ethenylene, 1-propenylene, 2-propenylene, 2-methyl-1-propenylene, 1-butenylene, 2-butenylene, and the like. In various embodiments, alkenylene groups can either be unsubstituted or substituted with one or more substituents. Typically, alkenylene groups will comprise 2 to 9 carbon atoms (for example, 2 to 6 carbon atoms, 2 to 4 carbon atoms, or 2 carbon atoms).

As used herein, “alkynylene” means a bivalent branched or straight chain chemical group containing only carbon and hydrogen and containing at least one carbon-carbon triple bond, such as ethynylene, 1-propynylene, 1-butynylene, 2-butynylene, and the like. In various embodiments, alkynylene groups can either be unsubstituted or substituted with one or more substituents. Typically, alkynylene groups will comprise 2 to 9 carbon atoms (for example, 2 to 6 carbon atoms, 2 to 4 carbon atoms, or 2 carbon atoms).

As used herein, “alkoxy” means an alkyl-O— group in which the alkyl group is as described herein. Exemplary alkoxy groups include methoxy, ethoxy, n-propoxy, i-propoxy, n-butoxy, s-butoxy, t-butoxy, pentoxy, hexoxy and heptoxy, and also the linear or branched positional isomers thereof.

As used herein, “haloalkoxy” means a haloalkyl-O— group in which the haloalkyl group is as described herein. Exemplary haloalkoxy groups include fluoromethoxy, difluoromethoxy, trifluoromethoxy, and also the linear or branched positional isomers thereof.

As used herein, “carbocyclyl” means a cyclic ring system containing only carbon atoms in the ring system backbone, such as cyclopropyl, cyclobutyl, cyclopentyl, cyclohexyl, and cyclohexenyl. Carbocyclyls may include multiple fused rings. Carbocyclyls may have any degree of saturation provided that none of the rings in the ring system are aromatic. Carbocyclyl groups can either be unsubstituted or substituted with one or more substituents. In some embodiments, carbocyclyl groups include 3 to 10 carbon atoms, for example, 3 to 6 carbon atoms.

As used herein, “aryl” means a mono-, bi-, tri- or polycyclic group with only carbon atoms present in the ring backbone having 5 to 14 ring atoms, alternatively 5, 6, 9, or 10 ring atoms; and having 6, 10, or 14 pi electrons shared in a cyclic array; wherein at least one ring in the system is aromatic. Aryl groups can either be unsubstituted or substituted with one or more substituents. Examples of aryl include phenyl, naphthyl, tetrahydronaphthyl, 2,3-dihydro-1H-indenyl, and others. In some embodiments, the aryl is phenyl.

As used herein, “arylalkylene” means an aryl-alkylene-group in which the aryl and alkylene moieties are as previously described. In some embodiments, arylalkylene groups contain a C₁₋₄alkylene moiety. Exemplary arylalkylene groups include benzyl and 2-phenethyl.

As used herein, the term “heteroaryl” means a mono-, bi-, tri- or polycyclic group having 5 to 14 ring atoms, alternatively 5, 6, 9, or 10 ring atoms; and having 6, 10, or 14 pi electrons shared in a cyclic array; wherein at least one ring in the system is aromatic, and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

As used herein, “halo”, “halide” or “halogen” is a chloro, bromo, fluoro, or iodo atom radical. In some embodiments, a halo is a chloro, bromo or fluoro. For example, a halide can be fluoro.

As used herein, “haloalkyl” means a hydrocarbon substituent, which is a linear or branched alkyl, alkenyl or alkynyl substituted with one or more chloro, bromo, fluoro, and/or iodo atom(s). In some embodiments, a haloalkyl is a fluoroalkyl, wherein one or more of the hydrogen atoms have been substituted by fluoro. In some embodiments, haloalkyls are 1 to 3 carbons in length (e.g., 1 to 2 carbons in length or 1 carbon in length). The term “haloalkylene” means a diradical variant of haloalkyl, and such diradicals may act as spacers between radicals, other atoms, or between a ring and another functional group.

As used herein, “heterocyclyl” means a nonaromatic cyclic ring system comprising at least one heteroatom in the ring system backbone. Heterocyclyls may include multiple fused rings such as bicyclic and spirocyclic heterocyclyls. Heterocyclyls may be substituted or unsubstituted with one or more substituents. In some embodiments, heterocycles have 3-11 members. In six membered monocyclic heterocycles, the heteroatom(s) are selected from one to three of O, N and S, and wherein when the heterocycle is five membered, it can have one or two heteroatoms selected from O, N, and S. Examples of heterocyclyl include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others. In some embodiments, the heterocyclyl is selected from azetidinyl, morpholinyl, piperazinyl, pyrrolidinyl, and tetrahydropyridinyl.

As used herein, “monocyclic heterocyclyl” means a single nonaromatic cyclic ring comprising at least one heteroatom in the ring system backbone. Heterocyclyls may be substituted or unsubstituted with one or more substituents. In some embodiments, heterocycles have 3-7 members. In six membered monocyclic heterocycles, the heteroatom(s) are selected from one to three of O, N and S, and wherein when the heterocycle is five membered, it can have one or two heteroatoms selected from O, N, and S. Examples of monocyclic heterocyclyls include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others.

As used herein, “bicyclic heterocyclyl” means a nonaromatic bicyclic ring system comprising at least one heteroatom in the ring system backbone. Bicyclic heterocyclyls may be substituted or unsubstituted with one or more substituents. In some embodiments, bicyclic heterocycles have 4-11 members with the heteroatom(s) being selected from one to five of O, N and S. Examples of bicyclic heterocyclyls include 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, and the like.

As used herein, “spirocyclic heterocyclyl” means a nonaromatic bicyclic ring system comprising at least one heteroatom in the ring system backbone and with the rings connected through just one atom. Spirocyclic heterocyclyls may be substituted or unsubstituted with one or more substituents. In some embodiments, spirocyclic heterocycles have 5-11 members with the heteroatom(s) being selected from one to five of O, N and S. Examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 2,5-diazaspiro[3.6]decane, and the like.

The term “substituted” refers to moieties having substituents replacing a hydrogen on one or more non-hydrogen atoms of the molecule. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Substituents can include, for example, —(C₁₋₉ alkyl) optionally substituted with one or more of hydroxyl, —NH₂, —NH(C₁₋₃ alkyl), and —N(C₁₋₃ alkyl)₂; —(C₁₋₉ haloalkyl); a halide; a hydroxyl; a carbonyl [such as —C(O)OR, and —C(O)R]; a thiocarbonyl [such as —C(S)OR, —C(O)SR, and —C(S)R]; —(C₁₋₉ alkoxy) optionally substituted with one or more of halide, hydroxyl, —NH₂, —NH(C₁₋₃ alkyl), and —N(C₁₋₃ alkyl)₂; —OPO(OH)₂; a phosphonate [such as —PO(OH)₂ and —PO(OR′)₂]; —OPO(OR′)R″; —NRR′; —C(O)NRR′; —C(NR)NR′R″; —C(NR′)R″; a cyano; a nitro; an azido; —SH; —S—R; —OSO₂(OR); a sulfonate [such as —SO₂(OH) and —SO₂(OR)]; —SO₂NR′R″; and —SO₂R; in which each occurrence of R, R′ and R″ are independently selected from H; —(C₁₋₉ alkyl); C₆₋₁₀ aryl optionally substituted with 1-3 R′″; 5-10 membered heteroaryl having from 1-4 heteroatoms independently selected from N, O, and S and optionally substituted with 1-3 R′″; C₃₋₇ carbocyclyl optionally substituted with 1-3 R′″; and 3-8 membered heterocyclyl having from 1-4 heteroatoms independently selected from N, O, and S and optionally substituted with 1-3 R′″; wherein each R′″ is independently selected from —(C₁₋₆ alkyl), —(C₁₋₆haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl). In some embodiments, the substituent is selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl).

As used herein, when two groups are indicated to be “linked” or “bonded” to form a “ring,” it is to be understood that a bond is formed between the two groups and may involve replacement of a hydrogen atom on one or both groups with the bond, thereby forming a carbocyclyl, heterocyclyl, aryl, or heteroaryl ring. The skilled artisan will recognize that such rings can and are readily formed by routine chemical reactions. In some embodiments, such rings have from 3-7 members, for example, 5 or 6 members.

The skilled artisan will recognize that some chemical structures described herein may be represented on paper by one or more other resonance forms; or may exist in one or more other tautomeric forms, even when kinetically, the artisan recognizes that such tautomeric forms represent only a very small portion of a sample of such compound(s). Such compounds are clearly contemplated within the scope of this disclosure, though such resonance forms or tautomers are not explicitly represented herein.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art. Unless otherwise indicated, when a disclosed compound is named or depicted by a structure without specifying the stereochemistry and has one or more chiral centers, it is understood to represent all possible stereoisomers of the compound.

The present disclosure includes all pharmaceutically acceptable isotopically labeled compounds of Formula I wherein one or more atoms are replaced by atoms having the same atomic number, but an atomic mass or mass number different from the atomic mass or mass number which predominates in nature. Examples of isotopes suitable for inclusion in the compounds of the disclosure include, but are not limited to, isotopes of hydrogen, such as ²H (deuterium) and ³H (tritium), isotopes of carbon, such as ¹¹C, ¹³C and ¹⁴C, isotopes of chlorine, such as ³⁶Cl, isotopes of fluorine, such as ¹⁸F, isotopes of iodine, such as ¹²³I and ¹²⁵I, isotopes of nitrogen, such as ¹³N and ¹⁵N, isotopes of oxygen, such as ¹⁵O, ¹⁷O and ¹⁸O, isotopes of phosphorus, such as ³²P, and isotopes of sulfur, such as ³⁵S.

The term “administration” or “administering” refers to a method of providing a dosage of a compound or pharmaceutical composition to a vertebrate or invertebrate, including a mammal, a bird, a fish, or an amphibian, where the method of administration is, e.g., orally, subcutaneously, intravenously, intralymphatic, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic device. The method of administration can vary depending on various factors, e.g., the components of the pharmaceutical composition, the site of the disease, the disease involved, and the severity of the disease.

A “diagnostic” as used herein is a compound, method, system, or device that assists in the identification or characterization of a health or disease state. The diagnostic can be used in standard assays as is known in the art.

The term “mammal” is used in its usual biological sense. Thus, it specifically includes humans, cattle, horses, monkeys, dogs, cats, mice, rats, cows, sheep, pigs, goats, and non-human primates, but also includes many other species.

The terms “pharmaceutically acceptable carrier,” “pharmaceutically acceptable diluent,” and “pharmaceutically acceptable excipient” include any and all solvents, co-solvents, complexing agents, dispersion media, coatings, isotonic and absorption delaying agents and the like which are not biologically or otherwise undesirable. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies.

The term “pharmaceutically acceptable salt” refers to salts that retain the biological effectiveness and properties of the compounds provided herein and, which are not biologically or otherwise undesirable. In many cases, the compounds provided herein are capable of forming acid and/or base salts by virtue of the presence of amino and/or carboxyl groups or groups similar thereto. Many such salts are known in the art, for example, as described in WO 87/05297. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

“Patient” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate. In some embodiments, the patient is a human.

A “therapeutically effective amount” of a compound as provided herein is one which is sufficient to achieve the desired physiological effect and may vary according to the nature and severity of the disease condition, and the potency of the compound. “Therapeutically effective amount” is also intended to include one or more of the compounds of Formula I in combination with one or more other agents that are effective to treat the diseases and/or conditions described herein. The combination of compounds can be a synergistic combination. Synergy, as described, for example, by Chou and Talalay, Advances in Enzyme Regulation (1984), 22, 27-55, occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon the patient's height, weight, sex, age, and medical history.

A therapeutic effect relieves, to some extent, one or more of the symptoms of the disease.

“Treat,” “treatment,” or “treating,” as used herein refers to administering a compound or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing, or preventing the further development of a disorder, and/or reducing the severity of symptoms that will or are expected to develop.

“Drug-eluting” and/or controlled release as used herein refers to any and all mechanisms, e.g., diffusion, migration, permeation, and/or desorption by which the drug(s) incorporated in the drug-eluting material pass therefrom overtime into the surrounding body tissue.

“Drug-eluting material” and/or controlled release material as used herein refers to any natural, synthetic, or semi-synthetic material capable of acquiring and retaining a desired shape or configuration and into which one or more drugs can be incorporated and from which incorporated drug(s) are capable of eluting over time.

“Elutable drug” as used herein refers to any drug or combination of drugs having the ability to pass over time from the drug-eluting material in which it is incorporated into the surrounding areas of the body.

### Compounds

The compounds and compositions described herein can be used to inhibit DYRK1A for treating a disorder or disease in which DYRK1A overexpression is implicated, such as in neurological diseases or disorders, cancers, cognitive deficits, knee osteoarthritis, tendinopathy, viral infections, unicellular parasite infections, and motor deficits.

Some embodiments of the present disclosure include compounds of Formula I:

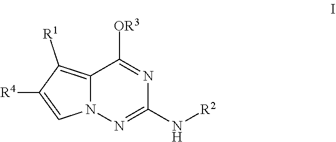

or salts, pharmaceutically acceptable salts, or prodrugs thereof.

In some embodiments of Formula I, R¹ is a 9-10 membered heteroaryl optionally substituted with 1-10 R⁵. In some embodiments, R¹ is a 9-10 membered heteroaryl optionally substituted with 1-10, 1-9, 1-8, 1-7, 1-6, 1-5, 1-4, 1-3, 1-2, or 1 R⁵.

In some embodiments of Formula I, R¹ is selected from the heteroaryl group consisting of:

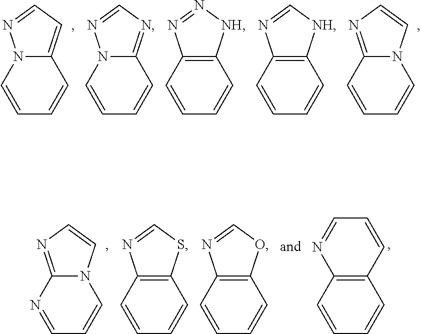

optionally substituted with 1-10 (e.g., 1-9, 1-8, 1-7, 1-6, 1-5, 1-4, 1-3, 1-2, 1) R⁵.

In some embodiments of Formula I, R¹ is selected from the heteroaryl group consisting of:

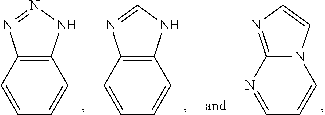

optionally substituted with 1-10 (e.g., 1-9, 1-8, 1-7, 1-6, 1-5, 1-4, 1-3, 1-2, 1) R⁵.

In some embodiments of Formula I, R¹ is selected from the heteroaryl group consisting of:

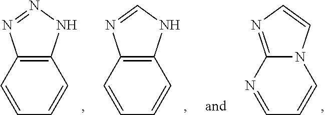

optionally substituted with 1-3 (e.g., 1-2, 1) R⁵.

In some embodiments of Formula I, R¹ is selected from the group consisting of

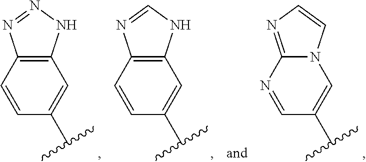 [SMILES: *c1ccc2nn[nH]c2c1 ; *c1ccc2nc[nH]c2c1 ; *c1cnc2nccn2c1]

optionally substituted with 1-3 R⁵.

In some embodiments of Formula I, R¹ is:

unsubstituted

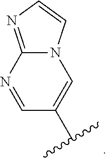 [SMILES: *c1cnc2nccn2c1]

In some embodiments of Formula I, R² is selected from the group consisting of —(C₁₋₅ alkylene)CN, -heterocyclyl optionally substituted with 1-10 (e.g., 1-9, 1-8, 1-7, 1-6, 1-5, 1-4, 1-3, 1-2, 1) R⁶, —(C₁₋₂ alkylene)C(═O)heterocyclyl, and -carbocyclyl optionally substituted with 1-12 (e.g., 1-11, 1-10, 1-9, 1-8, 1-7, 1-6, 1-5, 1-4, 1-3, 1-2, 1)R, wherein the —(C₁₋₂ alkylene) or (C₁₋₅ alkylene) is optionally substituted with 1-2 (e.g., 1) halide (e.g., F, Cl, Br, I) and/or 1-2 (e.g., 1) unsubstituted —(C₁₋₃ alkyl) (e.g., Me, Et, iPr, nPr).

In some embodiments of Formula I, R² is selected from the group consisting of —(C₁₋₅ alkylene)CN, -heterocyclyl optionally substituted with 1-4 (e.g., 1-3, 1-2, 1) R⁶, —CH₂C(═O)heterocyclyl, and -carbocyclyl optionally substituted with 1-4 (e.g., 1-3, 1-2, 1) R⁷, wherein the —(C₁₋₂ alkylene) or (C₁₋₅ alkylene) is optionally substituted with 1-2 halide.

In some embodiments of Formula I, R² is -carbocyclyl optionally substituted with 1-4 (e.g., 1-3, 1-2, 1) R⁷, wherein the carbocyclyl is selected from the group consisting of cyclopropyl, cyclobutyl, cyclopentyl, and cyclohexyl.

In some embodiments of Formula I, R² is selected from the group consisting of -heterocyclyl optionally substituted with 1-10 (e.g., 1-9, 1-8, 1-7, 1-6, 1-5, 1-4, 1-3, 1-2, 1) R⁶ and —(C₁₋₂ alkylene)C(═O)heterocyclyl, wherein the —(C₁₋₂ alkylene) is optionally substituted with 1-2 (e.g., 1) halide (e.g., F, Cl, Br, I) and/or 1-2 (e.g., 1) unsubstituted —(C₁₋₃ alkyl) (e.g., Me, Et, iPr, nPr).

In some embodiments of Formula I, R² is selected from the group consisting of -heterocyclyl optionally substituted with 1-4 (e.g., 1-3, 1-2, 1) R⁶ and —CH₂C(═O)heterocyclyl.

In some embodiments of Formula I, R² is selected from the group consisting of:

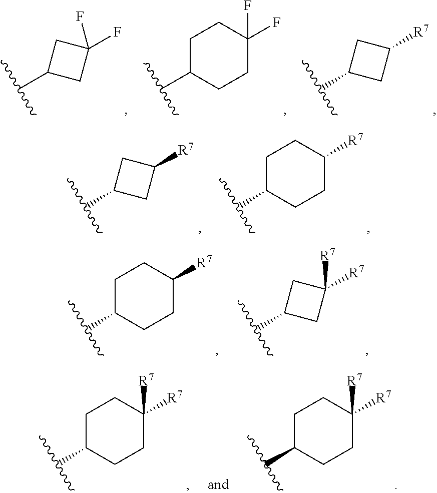

In some embodiments of Formula I, R² is —(C₁₋₂ alkylene)ₚheterocyclyl optionally substituted with 1-3 R⁶, wherein the —(C₁₋₅ alkylene) is optionally substituted with 1-2 halide and wherein the heterocyclyl is selected from the group consisting of oxetanyl, tetrahydrofuranyl, tetrahydropyranyl, azetidinyl, pyrrolidinyl, piperidinyl, morpholinyl, and piperazinyl.

In some embodiments of Formula I, R² is selected from the group consisting of:

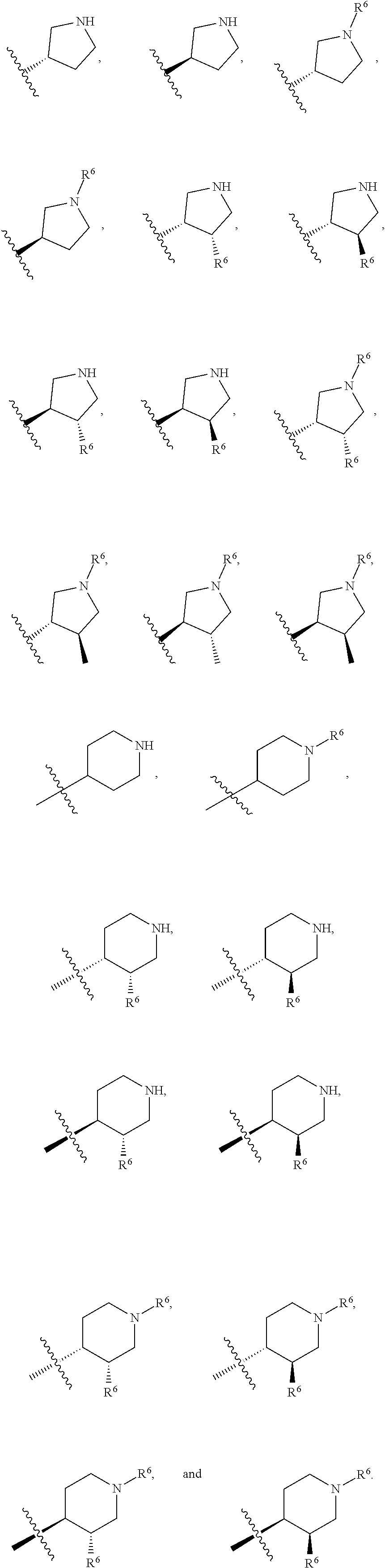

In some embodiments of Formula I, R³ is unsubstituted —(C₁₋₃ alkyl);

In some embodiments of Formula I, R³ is Me.

In some embodiments of Formula I, R⁴ is selected from the group consisting of H and halide (e.g., F, Cl, Br, I).

In some embodiments of Formula I, R⁴ is H.

In some embodiments of Formula I, R⁴ is halide.

In some embodiments of Formula I, R⁴ is F.

In some embodiments of Formula I, each R⁵ is independently selected from the group consisting of halide (e.g., F, Cl, Br, I), unsubstituted —(C₁₋₉ alkyl), unsubstituted —(C₁₋₉ haloalkyl), —(C₁₋₄ alkylene)OH, -carbocyclyl optionally substituted with 1-12 (e.g., 1-11, 1-10, 1-9, 1-8, 1-7, 1-6, 1-5, 1-4, 1-3, 1-2, 1) halides (e.g., F, Cl, Br, I), —C(═O)N(R⁸)₂, wherein the —(C₁₋₄ alkylene) is optionally substituted with 1-4 (e.g., 1-3, 1-2, 1) halide (e.g., F, Cl, Br, I) and/or 1-2 (e.g., 1) unsubstituted —(C₁₋₃ alkyl) (e.g., Me, Et, iPr, nPr).

In some embodiments of Formula I, each R⁵ is independently selected from the group consisting of F, unsubstituted —(C₁₋₄ alkyl), unsubstituted —(C₁₋₄ haloalkyl), —(C₁₋₃ alkylene)OH, -carbocyclyl optionally substituted with 1-4 fluorines, —C(═O)N(R⁸)₂, wherein the —(C₁₋₃ alkylene) is optionally substituted with 1-2 halide.

In some embodiments of Formula I, each R⁶ is independently selected from the group consisting of halide (e.g., F, Cl, Br, I), unsubstituted —(C₁₋₉ alkyl), unsubstituted -heterocyclyl, —(C₁₋₃ alkylene)OR⁹, —SO₂Me, and —(C₁₋₃ alkylene), C(═O)R¹⁰, wherein each —(C₁₋₃ alkylene) is, independently, optionally substituted with 1-2 (e.g., 1) halide (e.g., F, Cl, Br, I) and/or 1-2 (e.g., 1) unsubstituted —(C₁₋₃ alkyl) (e.g., Me, Et, iPr, nPr).

In some embodiments of Formula I, each R⁶ is independently selected from the group consisting of F, unsubstituted —(C₁₋₃ alkyl), unsubstituted -heterocyclyl, —CH₂CH₂OH, —CH₂CH₂OMe, —SO₂Me, and —(CH₂)ₚC(═O)R¹⁰.

In some embodiments of Formula I, alternatively, two R⁶ attached to the same carbon atom are taken together to form a carbonyl group.

In some embodiments of Formula I, each R⁶ is —(C₁₋₃ alkylene)ₚC(═O)R¹⁰, wherein the —(C₁₋₃ alkylene) is optionally substituted with 1-2 (e.g., 1) halide (e.g., F, Cl, Br, I) and/or 1-2 (e.g., 1) unsubstituted —(C₁₋₃ alkyl) (e.g., Me, Et, iPr, nPr).

In some embodiments of Formula I, each R⁶ is —(CH₂)ₚC(═O)R¹⁰.

In some embodiments of Formula I, each R¹ is independently selected from the group consisting of halide (e.g., F, Cl, Br, I), unsubstituted —(C₁₋₉ alkyl), —OH, and —NHC(═O)R¹¹.

In some embodiments of Formula I, each R¹ is independently selected from the group consisting of F, unsubstituted —(C₁₋₃ alkyl), —OH, and —NHC(═O)R¹¹.

In some embodiments of Formula I, each R⁸ is independently selected from the group consisting of H, unsubstituted —(C₁₋₉ alkyl), and unsubstituted —(C₁₋₉ haloalkyl).

In some embodiments of Formula I, each R⁸ is independently selected from the group consisting of H, unsubstituted —(C₁₋₃ alkyl), and unsubstituted —(C₁₋₃ haloalkyl).

In some embodiments of Formula I, each R⁹ is independently selected from the group consisting of H and unsubstituted —(C₁₋₉ alkyl).

In some embodiments of Formula I, each R⁹ is independently selected from the group consisting of H and unsubstituted —(C₁₋₃ alkyl).

In some embodiments of Formula I, each R⁹ is independently selected from the group consisting of H and Me.

In some embodiments of Formula I, each R¹⁰ is independently selected from the group consisting of unsubstituted —(C₁₋₉ alkyl), —(C₁₋₂ alkylene)ₚN(R¹²)₂, —(C₁₋₂ alkylene)OR⁹, —(C₁₋₂ alkylene)ₚheterocyclyl, and unsubstituted -carbocyclyl, wherein each —(C₁₋₂ alkylene) is, independently, optionally substituted with 1-2 (e.g., 1) halide (e.g., F, Cl, Br, I) and/or 1-2 (e.g., 1) unsubstituted —(C₁₋₃ alkyl) (e.g., Me, Et, iPr, nPr).

In some embodiments of Formula I, each R¹⁰ is independently selected from the group consisting of unsubstituted —(C₁₋₃ alkyl), —(CH₂)ₚN(R¹²)₂, —CH₂OR⁹, —(CH₂)ₚheterocyclyl, and unsubstituted -carbocyclyl.

In some embodiments of Formula I, each R¹⁰ is independently selected from the group consisting of unsubstituted —(C₁₋₃ alkyl), —(CH₂)ₚNMe₂, —CH₂OH, —(CH₂)ₚheterocyclyl, and unsubstituted -carbocyclyl.

In some embodiments of Formula I, each R¹⁰ is independently selected from the group consisting of —(C₁₋₂ alkylene)ₚN(R¹²)₂, —(C₁₋₂ alkylene)OR⁹, and —(C₁₋₂ alkylene)ₚheterocyclyl, wherein each —(C₁₋₂ alkylene) is, independently, optionally substituted with 1-2 (e.g., 1) halide (e.g., F, Cl, Br, I) and/or 1-2 (e.g., 1) unsubstituted —(C₁₋₃ alkyl) (e.g., Me, Et, iPr, nPr).

In some embodiments of Formula I, each R¹⁰ is independently selected from the group consisting of —(CH₂)ₚN(R¹²)₂, —CH₂OR⁹, and —(CH₂)ₚheterocyclyl.

In some embodiments of Formula I, each R¹⁰ is independently selected from the group consisting of —(CH₂)ₚNMe₂, —CH₂OH, —(CH₂)ₚheterocyclyl.

In some embodiments of Formula I, each R¹¹ is independently selected from the group consisting of unsubstituted —(C₁₋₉ alkyl), —(C₁₋₂ alkylene)ₚOR⁹, and -carbocyclyl optionally substituted with 1-12 (e.g., 1-11, 1-10, 1-9, 1-8, 1-7, 1-6, 1-5, 1-4, 1-3, 1-2, 1) halides (e.g., F, Cl, Br, I), wherein the —(C₁₋₂ alkylene) is optionally substituted with 1-2 (e.g., 1) halide (e.g., F, Cl, Br, I) and/or 1-2 (e.g., 1) unsubstituted —(C₁₋₃ alkyl) (e.g., Me, Et, iPr, nPr).

In some embodiments of Formula I, each R¹¹ is independently selected from the group consisting of unsubstituted —(C₁₋₃ alkyl), —CH₂OH, and -carbocyclyl optionally substituted with 1-2 (e.g., 1) halides (e.g., F, Cl, Br, I).

In some embodiments of Formula I, each R¹¹ is independently selected from the group consisting of Me, —CH₂OH, and -carbocyclyl optionally substituted with 1-2 fluorines.

In some embodiments of Formula I, each R¹² is independently selected from the group consisting of H and unsubstituted —(C₁₋₉ alkyl).

In some embodiments of Formula I, each R¹² is independently selected from the group consisting of H and unsubstituted —(C₁₋₃ alkyl).

In some embodiments of Formula I, each R¹² is independently selected from the group consisting of H and Me.

In some embodiments of Formula I, each p is independently 0 or 1.

In some embodiments of Formula I, each H atom is optionally, independently replaced by ²H (D) (deuterium).

In another embodiment disclosed herein includes a compound having the structure of Formula I:

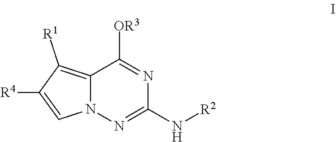

or a pharmaceutically acceptable salt thereof,
wherein:
R¹ is a 9-10 membered heteroaryl optionally substituted with 1-10 R⁵;
R² is selected from -heterocyclyl substituted with 1-10 R^{6a} and optionally with 1-10 R^{6b}; or —(C₁₋₂ alkylene)C(═O)heterocyclyl, wherein the —(C₁₋₂ alkylene) is optionally substituted with 1-2 halide and/or 1-2 unsubstituted —(C₁₋₃ alkyl);
R³ is unsubstituted —(C₁₋₃ alkyl);
R⁴ is selected from the group consisting of H and halide;
each R⁵ is independently selected from the group consisting of halide, unsubstituted —(C₁₋₉ alkyl), unsubstituted —(C₁₋₉ haloalkyl), —(C₁₋₄ alkylene)OH, -carbocyclyl optionally substituted with 1-12 halides, —C(═O)N(R)₂, wherein the —(C₁₋₄ alkylene) is optionally substituted with 1-4 halide and/or 1-2 unsubstituted —(C₁₋₃ alkyl);
each R^{6a} is —(C₁₋₃ alkylene)C(═O)R¹⁰, wherein —(C₁₋₃ alkylene) is optionally substituted with 1-2 halide and/or 1-2 unsubstituted —(C₁₋₃ alkyl);
each R^{6b} is halide;
each R⁸ is independently selected from the group consisting of H, unsubstituted —(C₁₋₉ alkyl), and unsubstituted —(C₁₋₉ haloalkyl);
each R⁹ is independently selected from the group consisting of H and unsubstituted —(C₁₋₉ alkyl);
each R¹⁰ is independently selected from the group consisting of unsubstituted —(C₁₋₉ alkyl), —(C₁₋₂ alkylene)ₚN(R¹²)₂, —(C₁₋₂ alkylene)OR⁹, —(C₁₋₂ alkylene)ₚheterocyclyl, and unsubstituted -carbocyclyl, wherein each —(C₁₋₂ alkylene) is, independently, optionally substituted with 1-2 halide and/or 1-2 unsubstituted —(C₁₋₃ alkyl);
each R¹² is independently selected from the group consisting of H and unsubstituted —(C₁-9 alkyl);
each p is independently 0 or 1; and
wherein each H atom is optionally, independently, replaced by ²H (D) (deuterium).

In another embodiment disclosed herein includes a compound having the structure of Formula I:

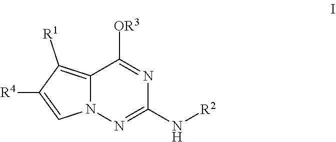

or a pharmaceutically acceptable salt thereof,
wherein:
R¹ is a 9-10 membered heteroaryl optionally substituted with 1-3 R⁵;
R² is selected from -heterocyclyl substituted with 1 R^{6a} and with 1-4 R^{6b}; or —(C₁₋₂ alkylene)C(═O)heterocyclyl, wherein the —(C₁₋₂ alkylene) is optionally substituted with 1-2 halide and/or 1-2 unsubstituted —(C₁₋₃ alkyl);
R³ is unsubstituted —(C₁₋₂ alkyl);
R⁴ is selected from the group consisting of H and halide;
each R⁵ is independently selected from the group consisting of halide, unsubstituted —(C₁₋₃ alkyl), unsubstituted —(C₁₋₄ haloalkyl), —(C₁₋₂ alkylene)OH, -carbocyclyl optionally substituted with 1-4 halides, —C(═O)N(RW)₂, wherein the —(C₁₋₄ alkylene) is optionally substituted with 1-4 halide and/or 1-2 unsubstituted —(C₁₋₃ alkyl);
each R^{6a} is —(C₁₋₃ alkylene)C(═O)R¹⁰, wherein —(C₁₋₃ alkylene) is optionally substituted with 1-2 halide and/or 1-2 unsubstituted —(C₁₋₃ alkyl);
each R^{6b} is halide;
each R⁸ is independently selected from the group consisting of H, unsubstituted —(C₁₋₃ alkyl), and unsubstituted —(C₁₋₃ haloalkyl);
each R⁹ is independently selected from the group consisting of H and unsubstituted —(C₁₋₃ alkyl);
each R¹⁰ is independently selected from the group consisting of unsubstituted —(C₁₋₃ alkyl), —(C₁₋₂ alkylene)ₚN(R¹²)₂, —(C₁₋₂ alkylene)OR⁹, —(C₁₋₂ alkylene)ₚheterocyclyl, and unsubstituted -carbocyclyl, wherein each —(C₁₋₂ alkylene) is, independently, optionally substituted with 1-2 halide and/or 1-2 unsubstituted —(C₁₋₃ alkyl);
each R¹² is independently selected from the group consisting of H and unsubstituted —(C₁₋₃ alkyl);
each p is independently 0 or 1; and
wherein each H atom is optionally, independently, replaced by ²H (D) (deuterium).

In some embodiments of Formula I, R² is —(CH₂)C(═O)(unsubstituted)heterocyclyl.

In some embodiments of Formula I, each R^{6a} is —(CH₂)C(═O)R¹⁰

In some embodiments of Formula I, each R^{6a} is —(CH₂)C(═O)R¹⁰, wherein R¹⁰ is selected the group consisting of —N(R¹²)₂ and unsubstituted -heterocyclyl.

In some embodiments of Formula I, each R^{6b} is F.

Illustrative compounds of Formula I are shown in Table 1.

TABLE 1

| | |
|---|---|
| 1 | 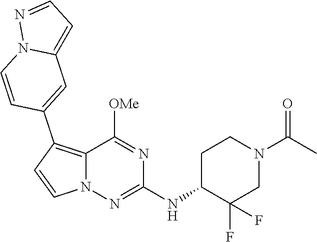 [SMILES: COc1nc(N[C@@H]2CCN(C(C)=O)CC2(F)F)nn2ccc(-c3ccn4nccc4c3)c12] |
| 2 | 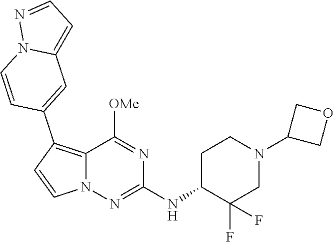 [SMILES: COc1nc(N[C@@H]2CCN(C3COC3)CC2(F)F)nn2ccc(-c3ccn4nccc4c3)c12] |
| 3 | 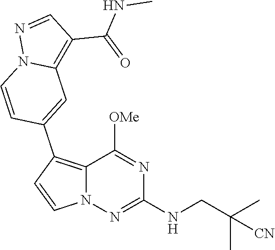 [SMILES: CNC(=O)c1cnn2ccc(-c3ccn4nc(NCC(C)(C)C#N)nc(OC)c34)cc12] |
| 4 | 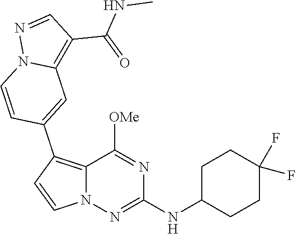 [SMILES: CNC(=O)c1cnn2ccc(-c3ccn4nc(NC5CCC(F)(F)CC5)nc(OC)c34)cc12] |
| 5 | 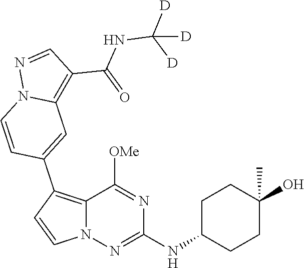 [SMILES: [2H]C([2H])([2H])NC(=O)c1cnn2ccc(-c3ccn4nc(N[C@H]5CC[C@](C)(O)CC5)nc(OC)c34)cc12] |
| 6 | 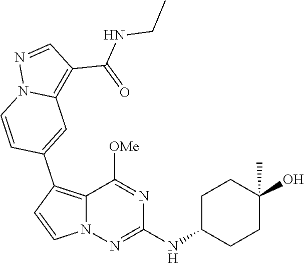 [SMILES: CCNC(=O)c1cnn2ccc(-c3ccn4nc(N[C@H]5CC[C@](C)(O)CC5)nc(OC)c34)cc12] |
| 7 | 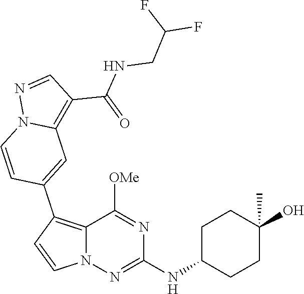 [SMILES: COc1nc(N[C@H]2CC[C@](C)(O)CC2)nn2ccc(-c3ccn4ncc(C(=O)NCC(F)F)c4c3)c12] |
| 8 | 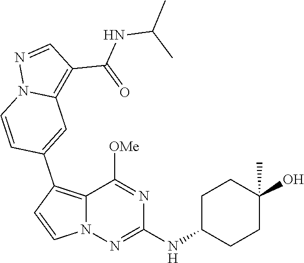 [SMILES: COc1nc(N[C@H]2CC[C@](C)(O)CC2)nn2ccc(-c3ccn4ncc(C(=O)NC(C)C)c4c3)c12] |
| 9 | 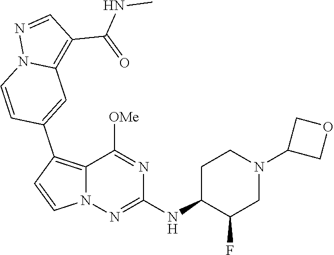 [SMILES: CNC(=O)c1cnn2ccc(-c3ccn4nc(N[C@H]5CCN(C6COC6)C[C@H]5F)nc(OC)c34)cc12] |
| 10 | 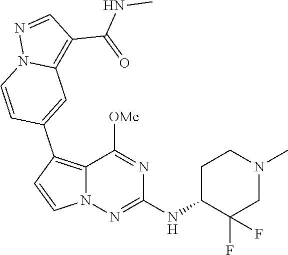 [SMILES: CNC(=O)c1cnn2ccc(-c3ccn4nc(N[C@@H]5CCN(C)CC5(F)F)nc(OC)c34)cc12] |
| 11 | 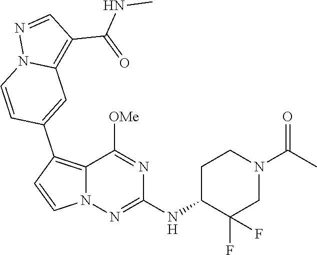 [SMILES: CNC(=O)c1cnn2ccc(-c3ccn4nc(N[C@@H]5CCN(C(C)=O)CC5(F)F)nc(OC)c34)cc12] |
| 12 | 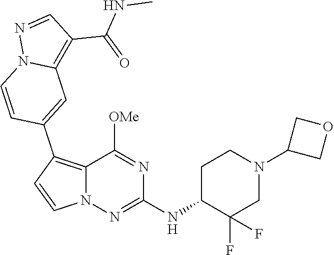 [SMILES: CNC(=O)c1cnn2ccc(-c3ccn4nc(N[C@@H]5CCN(C6COC6)CC5(F)F)nc(OC)c34)cc12] |
| 13 | 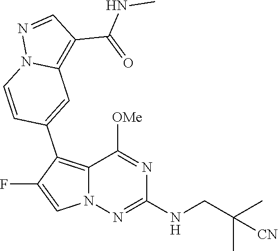 [SMILES: CNC(=O)c1cnn2ccc(-c3c(F)cn4nc(NCC(C)(C)C#N)nc(OC)c34)cc12] |
| 14 | 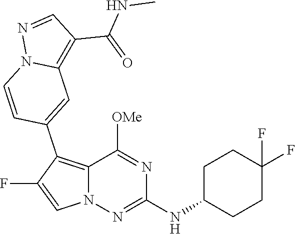 |
| 15 | 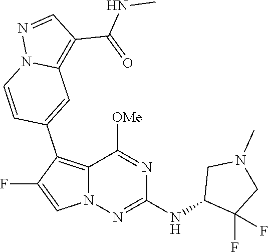 [SMILES: CNC(=O)c1cnn2ccc(-c3c(F)cn4nc(N[C@@H]5CN(C)CC5(F)F)nc(OC)c34)cc12] |
| 16 | 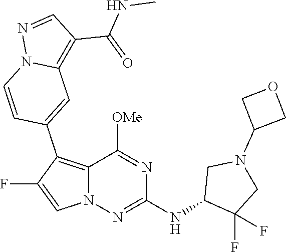 [SMILES: CNC(=O)c1cnn2ccc(-c3c(F)cn4nc(N[C@@H]5CN(C6COC6)CC5(F)F)nc(OC)c34)cc12] |
| 17 | 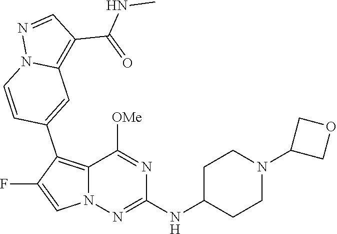 [SMILES: CNC(=O)c1cnn2ccc(-c3c(F)cn4nc(NC5CCN(C6COC6)CC5)nc(OC)c34)cc12] |
| 18 | 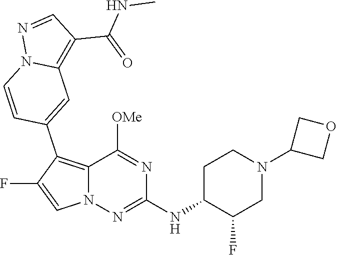 [SMILES: CNC(=O)c1cnn2ccc(-c3c(F)cn4nc(N[C@@H]5CCN(C6COC6)C[C@@H]5F)nc(OC)c34)cc12] |
| 19 | 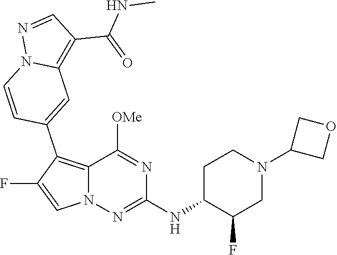 [SMILES: CNC(=O)c1cnn2ccc(-c3c(F)cn4nc(N[C@@H]5CCN(C6COC6)C[C@H]5F)nc(OC)c34)cc12] |
| 20 | 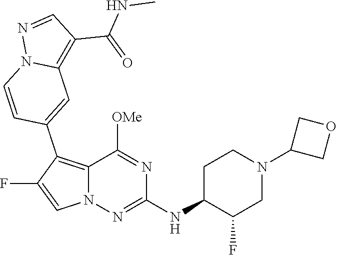 [SMILES: CNC(=O)c1cnn2ccc(-c3c(F)cn4nc(N[C@H]5CCN(C6COC6)C[C@@H]5F)nc(OC)c34)cc12] |
| 21 | 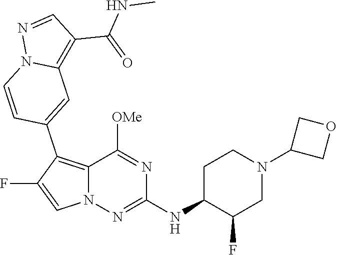 [SMILES: CNC(=O)c1cnn2ccc(-c3c(F)cn4nc(N[C@H]5CCN(C6COC6)C[C@H]5F)nc(OC)c34)cc12] |
| 22 | 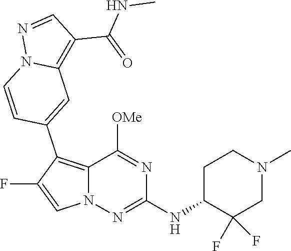 [SMILES: CNC(=O)c1cnn2ccc(-c3c(F)cn4nc(N[C@@H]5CCN(C)CC5(F)F)nc(OC)c34)cc12] |
| 23 | 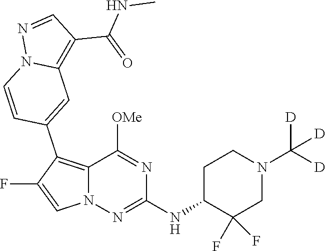 [SMILES: [2H]C([2H])([2H])N1CC[C@@H](Nc2nc(OC)c3c(-c4ccn5ncc(C(=O)NC)c5c4)c(F)cn3n2)C(F)(F)C1] |
| 24 | 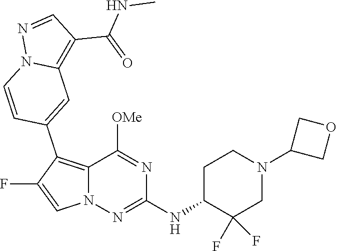 [SMILES: CNC(=O)c1cnn2ccc(-c3c(F)cn4nc(N[C@@H]5CCN(C6COC6)CC5(F)F)nc(OC)c34)cc12] |
| 25 | 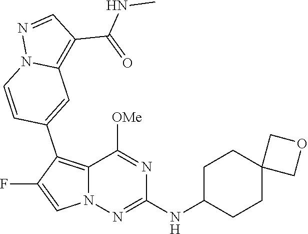 [SMILES: CNC(=O)c1cnn2ccc(-c3c(F)cn4nc(NC5CCC6(CC5)COC6)nc(OC)c34)cc12] |
| 26 | 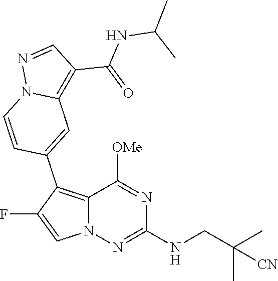 [SMILES: COc1nc(NCC(C)(C)C#N)nn2cc(F)c(-c3ccn4ncc(C(=O)NC(C)C)c4c3)c12] |
| 27 | 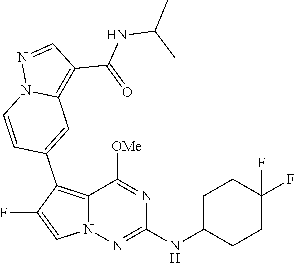 [SMILES: COc1nc(NC2CCC(F)(F)CC2)nn2cc(F)c(-c3ccn4ncc(C(=O)NC(C)C)c4c3)c12] |
| 28 | 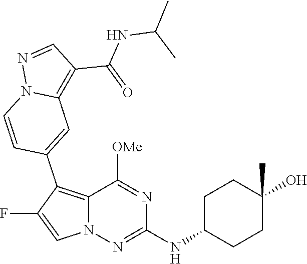 [SMILES: COc1nc(N[C@H]2CC[C@@](C)(O)CC2)nn2cc(F)c(-c3ccn4ncc(C(=O)NC(C)C)c4c3)c12] |
| 29 | 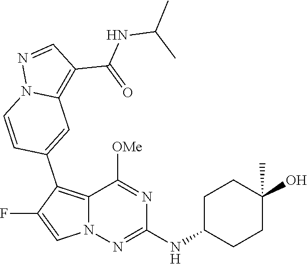 [SMILES: COc1nc(N[C@H]2CC[C@](C)(O)CC2)nn2cc(F)c(-c3ccn4ncc(C(=O)NC(C)C)c4c3)c12] |
| 30 | 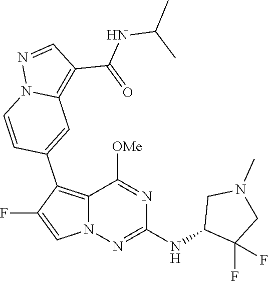 [SMILES: COc1nc(N[C@@H]2CN(C)CC2(F)F)nn2cc(F)c(-c3ccn4ncc(C(=O)NC(C)C)c4c3)c12] |
| 31 | 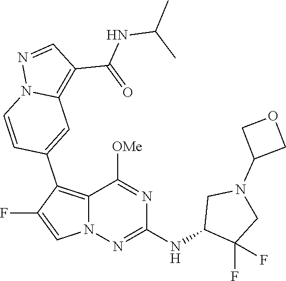 [SMILES: COc1nc(N[C@@H]2CN(C3COC3)CC2(F)F)nn2cc(F)c(-c3ccn4ncc(C(=O)NC(C)C)c4c3)c12] |
| 32 | 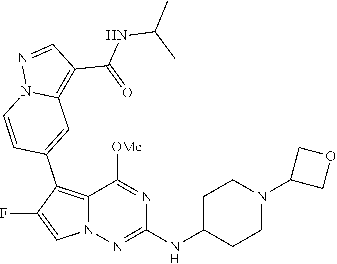 [SMILES: COc1nc(NC2CCN(C3COC3)CC2)nn2cc(F)c(-c3ccn4ncc(C(=O)NC(C)C)c4c3)c12] |
| 33 | 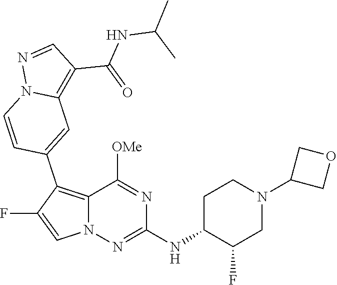 [SMILES: COc1nc(N[C@@H]2CCN(C3COC3)C[C@@H]2F)nn2cc(F)c(-c3ccn4ncc(C(=O)NC(C)C)c4c3)c12] |
| 34 | 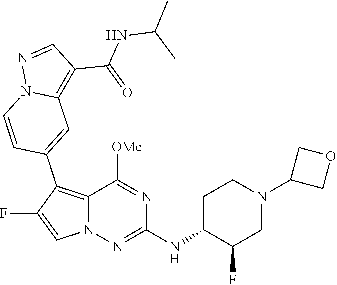 [SMILES: COc1nc(N[C@@H]2CCN(C3COC3)C[C@H]2F)nn2cc(F)c(-c3ccn4ncc(C(=O)NC(C)C)c4c3)c12] |
| 35 | 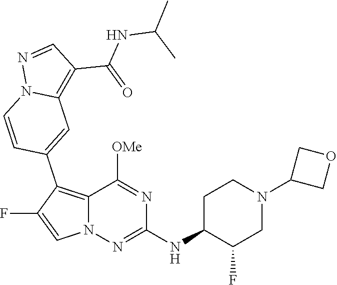 [SMILES: COc1nc(N[C@H]2CCN(C3COC3)C[C@@H]2F)nn2cc(F)c(-c3ccn4ncc(C(=O)NC(C)C)c4c3)c12] |
| 36 | 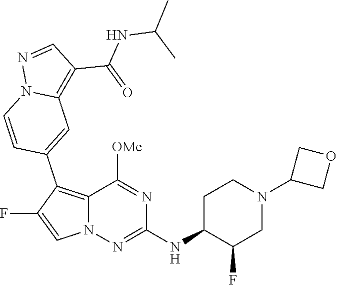 [SMILES: COc1nc(N[C@H]2CCN(C3COC3)C[C@H]2F)nn2cc(F)c(-c3ccn4ncc(C(=O)NC(C)C)c4c3)c12] |
| 37 | 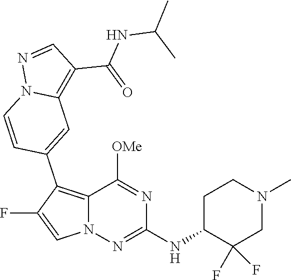 [SMILES: COc1nc(N[C@@H]2CCN(C)CC2(F)F)nn2cc(F)c(-c3ccn4ncc(C(=O)NC(C)C)c4c3)c12] |
| 38 | 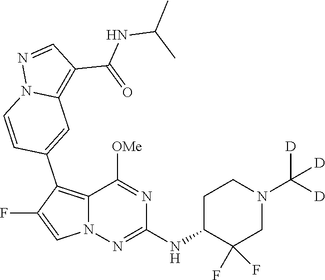 [SMILES: [2H]C([2H])([2H])N1CC[C@@H](Nc2nc(OC)c3c(-c4ccn5ncc(C(=O)NC(C)C)c5c4)c(F)cn3n2)C(F)(F)C1] |
| 39 | 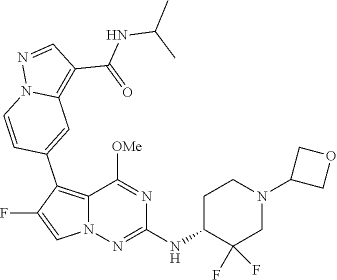 [SMILES: COc1nc(N[C@@H]2CCN(C3COC3)CC2(F)F)nn2cc(F)c(-c3ccn4ncc(C(=O)NC(C)C)c4c3)c12] |
| 40 | 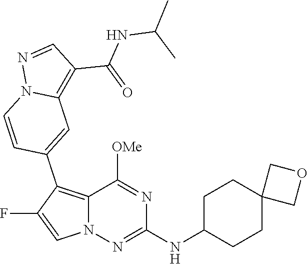 [SMILES: COc1nc(NC2CCC3(CC2)COC3)nn2cc(F)c(-c3ccn4ncc(C(=O)NC(C)C)c4c3)c12] |
| 41 | 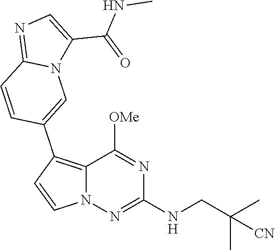 [SMILES: CNC(=O)c1cnc2ccc(-c3ccn4nc(NCC(C)(C)C#N)nc(OC)c34)cn12] |
| 42 | 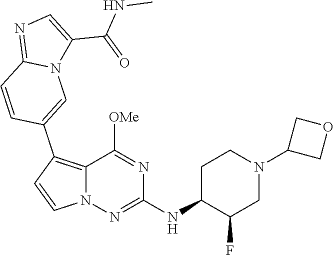 [SMILES: CNC(=O)c1cnc2ccc(-c3ccn4nc(N[C@H]5CCN(C6COC6)C[C@H]5F)nc(OC)c34)cn12] |
| 43 | 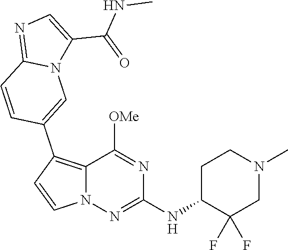 [SMILES: CNC(=O)c1cnc2ccc(-c3ccn4nc(N[C@@H]5CCN(C)CC5(F)F)nc(OC)c34)cn12] |
| 44 | 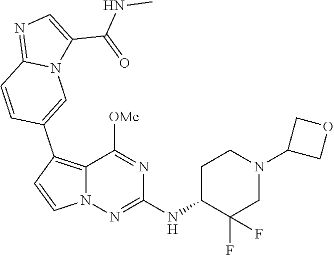 [SMILES: CNC(=O)c1cnc2ccc(-c3ccn4nc(N[C@@H]5CCN(C6COC6)CC5(F)F)nc(OC)c34)cn12] |
| 45 | 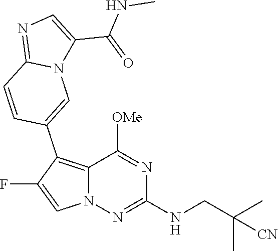 [SMILES: CNC(=O)c1cnc2ccc(-c3c(F)cn4nc(NCC(C)(C)C#N)nc(OC)c34)cn12] |
| 46 | 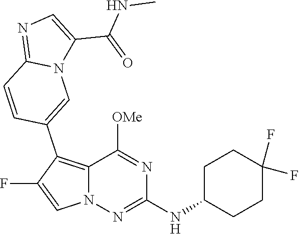 |
| 47 | 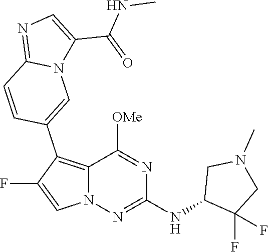 [SMILES: CNC(=O)c1cnc2ccc(-c3c(F)cn4nc(N[C@@H]5CN(C)CC5(F)F)nc(OC)c34)cn12] |
| 48 | 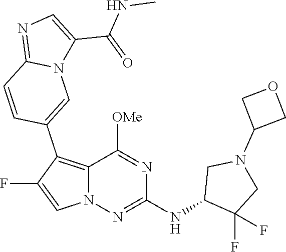 [SMILES: CNC(=O)c1cnc2ccc(-c3c(F)cn4nc(N[C@@H]5CN(C6COC6)CC5(F)F)nc(OC)c34)cn12] |
| 49 | 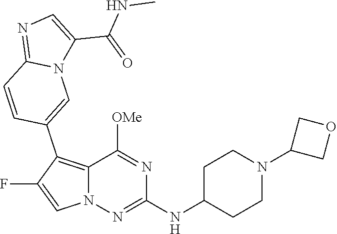 [SMILES: CNC(=O)c1cnc2ccc(-c3c(F)cn4nc(NC5CCN(C6COC6)CC5)nc(OC)c34)cn12] |
| 50 | 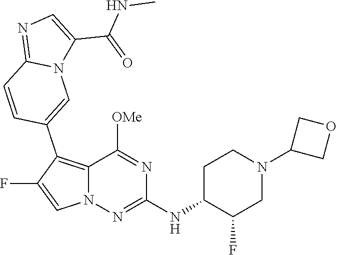 [SMILES: CNC(=O)c1cnc2ccc(-c3c(F)cn4nc(N[C@@H]5CCN(C6COC6)C[C@@H]5F)nc(OC)c34)cn12] |
| 51 | 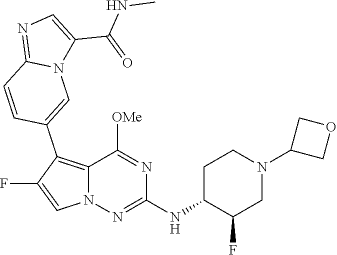 [SMILES: CNC(=O)c1cnc2ccc(-c3c(F)cn4nc(N[C@@H]5CCN(C6COC6)C[C@H]5F)nc(OC)c34)cn12] |
| 52 | 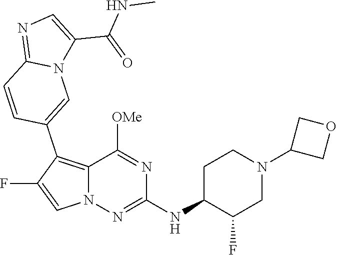 [SMILES: CNC(=O)c1cnc2ccc(-c3c(F)cn4nc(N[C@H]5CCN(C6COC6)C[C@@H]5F)nc(OC)c34)cn12] |
| 53 | 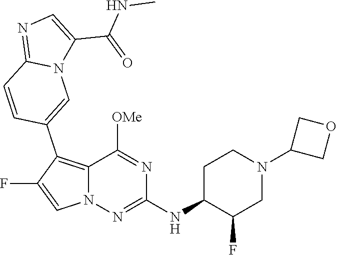 [SMILES: CNC(=O)c1cnc2ccc(-c3c(F)cn4nc(N[C@H]5CCN(C6COC6)C[C@H]5F)nc(OC)c34)cn12] |
| 54 | 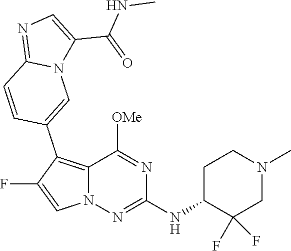 [SMILES: CNC(=O)c1cnc2ccc(-c3c(F)cn4nc(N[C@@H]5CCN(C)CC5(F)F)nc(OC)c34)cn12] |
| 55 | 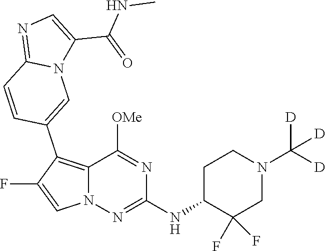 [SMILES: [2H]C([2H])([2H])N1CC[C@@H](Nc2nc(OC)c3c(-c4ccc5ncc(C(=O)NC)n5c4)c(F)cn3n2)C(F)(F)C1] |
| 56 | 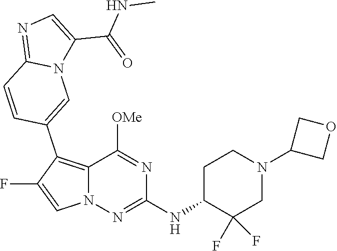 [SMILES: CNC(=O)c1cnc2ccc(-c3c(F)cn4nc(N[C@@H]5CCN(C6COC6)CC5(F)F)nc(OC)c34)cn12] |
| 57 | 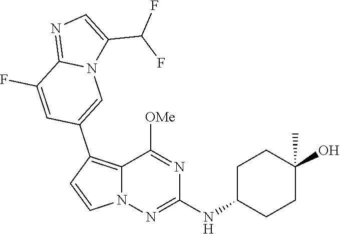 [SMILES: COc1nc(N[C@H]2CC[C@](C)(O)CC2)nn2ccc(-c3cc(F)c4ncc(C(F)F)n4c3)c12] |
| 58 | 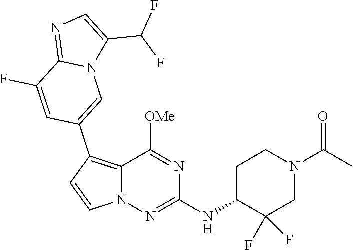 [SMILES: COc1nc(N[C@@H]2CCN(C(C)=O)CC2(F)F)nn2ccc(-c3cc(F)c4ncc(C(F)F)n4c3)c12] |
| 59 | 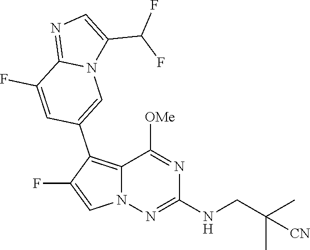 [SMILES: COc1nc(NCC(C)(C)C#N)nn2cc(F)c(-c3cc(F)c4ncc(C(F)F)n4c3)c12] |
| 60 | 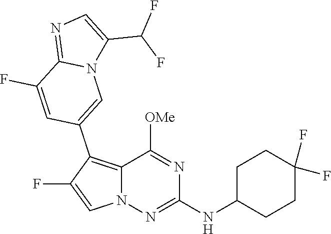 [SMILES: COc1nc(NC2CCC(F)(F)CC2)nn2cc(F)c(-c3cc(F)c4ncc(C(F)F)n4c3)c12] |
| 61 | 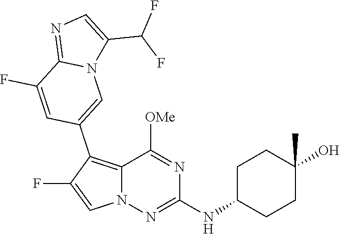 [SMILES: COc1nc(N[C@H]2CC[C@@](C)(O)CC2)nn2cc(F)c(-c3cc(F)c4ncc(C(F)F)n4c3)c12] |
| 62 | 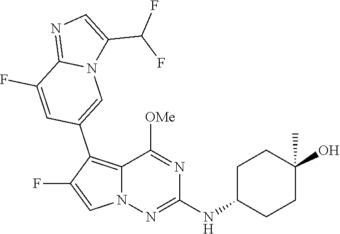 [SMILES: COc1nc(N[C@H]2CC[C@](C)(O)CC2)nn2cc(F)c(-c3cc(F)c4ncc(C(F)F)n4c3)c12] |
| 63 | 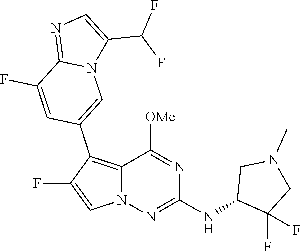 [SMILES: COc1nc(N[C@@H]2CN(C)CC2(F)F)nn2cc(F)c(-c3cc(F)c4ncc(C(F)F)n4c3)c12] |
| 64 | 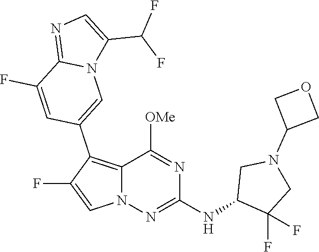 [SMILES: COc1nc(N[C@@H]2CN(C3COC3)CC2(F)F)nn2cc(F)c(-c3cc(F)c4ncc(C(F)F)n4c3)c12] |
| 65 | 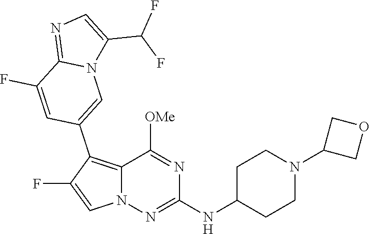 [SMILES: COc1nc(NC2CCN(C3COC3)CC2)nn2cc(F)c(-c3cc(F)c4ncc(C(F)F)n4c3)c12] |
| 66 | 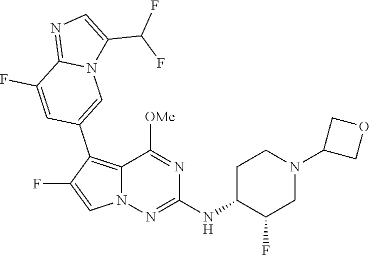 [SMILES: COc1nc(N[C@@H]2CCN(C3COC3)C[C@@H]2F)nn2cc(F)c(-c3cc(F)c4ncc(C(F)F)n4c3)c12] |
| 67 | 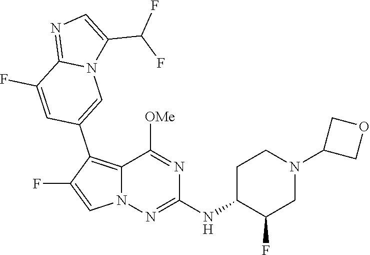 [SMILES: COc1nc(N[C@@H]2CCN(C3COC3)C[C@H]2F)nn2cc(F)c(-c3cc(F)c4ncc(C(F)F)n4c3)c12] |
| 68 | 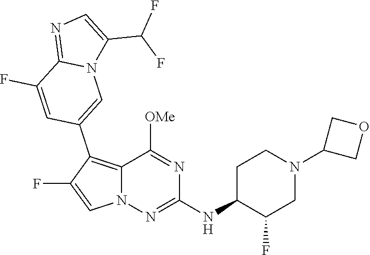 [SMILES: COc1nc(N[C@H]2CCN(C3COC3)C[C@@H]2F)nn2cc(F)c(-c3cc(F)c4ncc(C(F)F)n4c3)c12] |
| 69 | 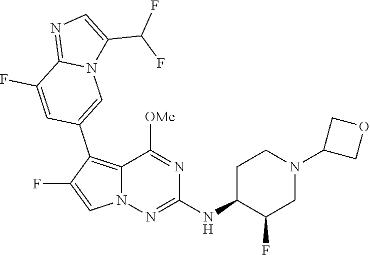 [SMILES: COc1nc(N[C@H]2CCN(C3COC3)C[C@H]2F)nn2cc(F)c(-c3cc(F)c4ncc(C(F)F)n4c3)c12] |
| 70 | 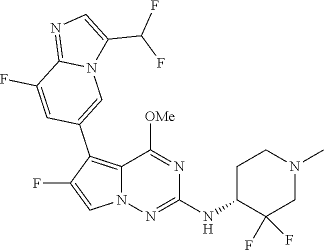 [SMILES: COc1nc(N[C@@H]2CCN(C)CC2(F)F)nn2cc(F)c(-c3cc(F)c4ncc(C(F)F)n4c3)c12] |
| 71 | 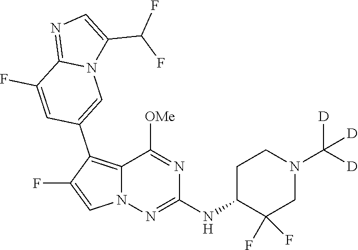 [SMILES: [2H]C([2H])([2H])N1CC[C@@H](Nc2nc(OC)c3c(-c4cc(F)c5ncc(C(F)F)n5c4)c(F)cn3n2)C(F)(F)C1] |
| 72 | 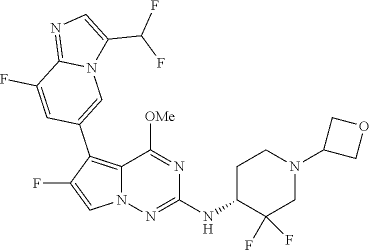 [SMILES: COc1nc(N[C@@H]2CCN(C3COC3)CC2(F)F)nn2cc(F)c(-c3cc(F)c4ncc(C(F)F)n4c3)c12] |
| 73 | 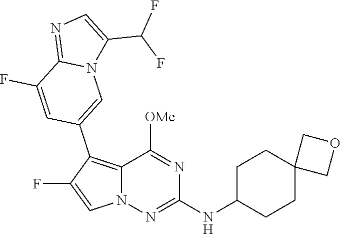 [SMILES: COc1nc(NC2CCC3(CC2)COC3)nn2cc(F)c(-c3cc(F)c4ncc(C(F)F)n4c3)c12] |
| 74 | 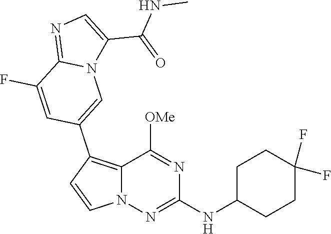 [SMILES: CNC(=O)c1cnc2c(F)cc(-c3ccn4nc(NC5CCC(F)(F)CC5)nc(OC)c34)cn12] |
| 75 | 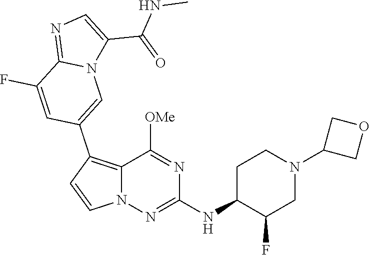 [SMILES: CNC(=O)c1cnc2c(F)cc(-c3ccn4nc(N[C@H]5CCN(C6COC6)C[C@H]5F)nc(OC)c34)cn12] |
| 76 | 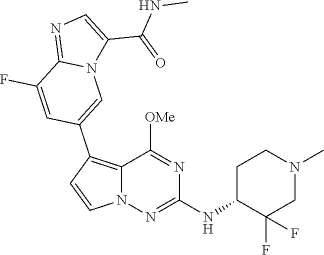 [SMILES: CNC(=O)c1cnc2c(F)cc(-c3ccn4nc(N[C@@H]5CCN(C)CC5(F)F)nc(OC)c34)cn12] |
| 77 | 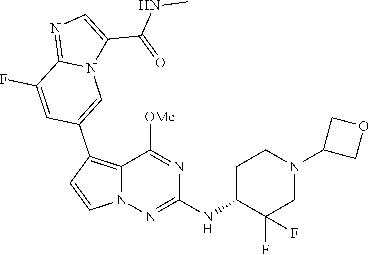 [SMILES: CNC(=O)c1cnc2c(F)cc(-c3ccn4nc(N[C@@H]5CCN(C6COC6)CC5(F)F)nc(OC)c34)cn12] |
| 78 | 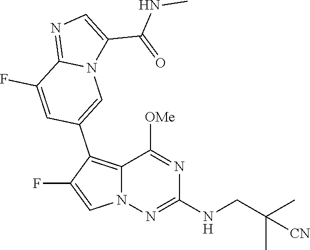 [SMILES: CNC(=O)c1cnc2c(F)cc(-c3c(F)cn4nc(NCC(C)(C)C#N)nc(OC)c34)cn12] |
| 79 | 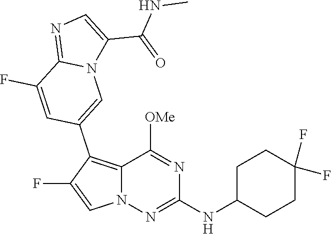 [SMILES: CNC(=O)c1cnc2c(F)cc(-c3c(F)cn4nc(NC5CCC(F)(F)CC5)nc(OC)c34)cn12] |
| 80 | 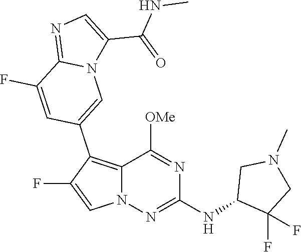 [SMILES: CNC(=O)c1cnc2c(F)cc(-c3c(F)cn4nc(N[C@@H]5CN(C)CC5(F)F)nc(OC)c34)cn12] |
| 81 | 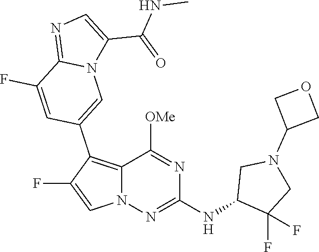 [SMILES: CNC(=O)c1cnc2c(F)cc(-c3c(F)cn4nc(N[C@@H]5CN(C6COC6)CC5(F)F)nc(OC)c34)cn12] |
| 82 | 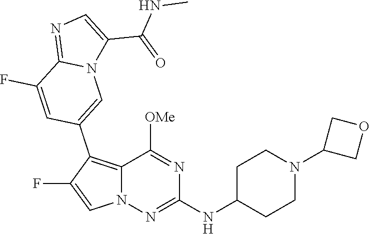 [SMILES: CNC(=O)c1cnc2c(F)cc(-c3c(F)cn4nc(NC5CCN(C6COC6)CC5)nc(OC)c34)cn12] |
| 83 | 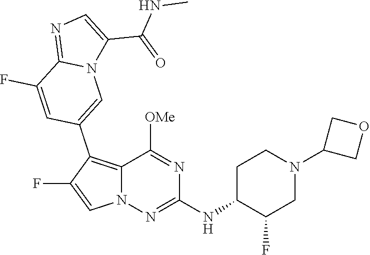 [SMILES: CNC(=O)c1cnc2c(F)cc(-c3c(F)cn4nc(N[C@@H]5CCN(C6COC6)C[C@@H]5F)nc(OC)c34)cn12] |
| 84 | 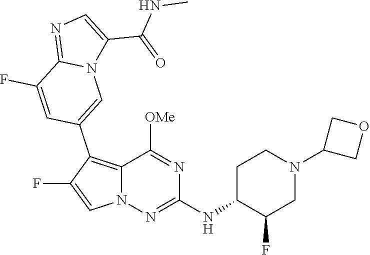 [SMILES: CNC(=O)c1cnc2c(F)cc(-c3c(F)cn4nc(N[C@@H]5CCN(C6COC6)C[C@H]5F)nc(OC)c34)cn12] |
| 85 | 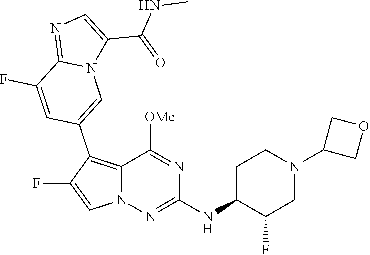 [SMILES: CNC(=O)c1cnc2c(F)cc(-c3c(F)cn4nc(N[C@H]5CCN(C6COC6)C[C@@H]5F)nc(OC)c34)cn12] |
| 86 | 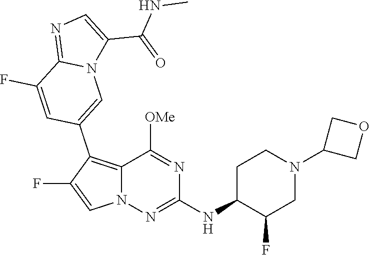 [SMILES: CNC(=O)c1cnc2c(F)cc(-c3c(F)cn4nc(N[C@H]5CCN(C6COC6)C[C@H]5F)nc(OC)c34)cn12] |
| 87 | 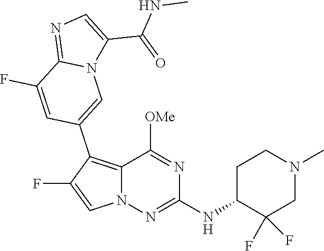 [SMILES: CNC(=O)c1cnc2c(F)cc(-c3c(F)cn4nc(N[C@@H]5CCN(C)CC5(F)F)nc(OC)c34)cn12] |
| 88 | 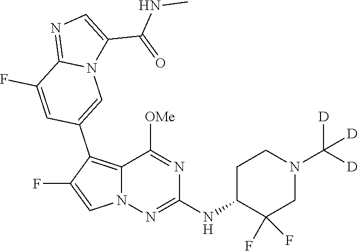 [SMILES: [2H]C([2H])([2H])N1CC[C@@H](Nc2nc(OC)c3c(-c4cc(F)c5ncc(C(=O)NC)n5c4)c(F)cn3n2)C(F)(F)C1] |
| 89 | 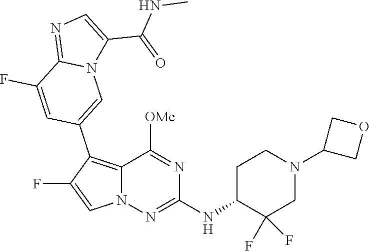 [SMILES: CNC(=O)c1cnc2c(F)cc(-c3c(F)cn4nc(N[C@@H]5CCN(C6COC6)CC5(F)F)nc(OC)c34)cn12] |
| 90 | 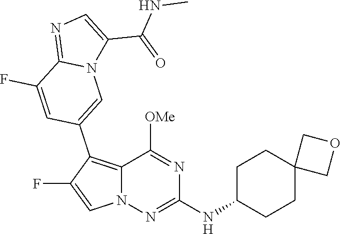 |
| 91 | 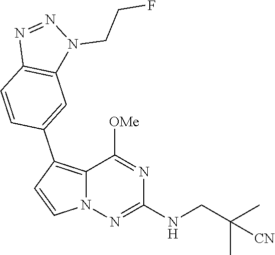 [SMILES: COc1nc(NCC(C)(C)C#N)nn2ccc(-c3ccc4nnn(CCF)c4c3)c12] |
| 92 | 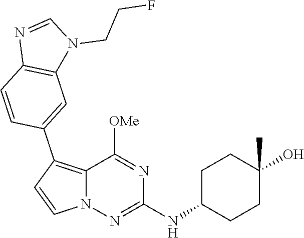 [SMILES: COc1nc(N[C@H]2CC[C@@](C)(O)CC2)nn2ccc(-c3ccc4ncn(CCF)c4c3)c12] |
| 93 | 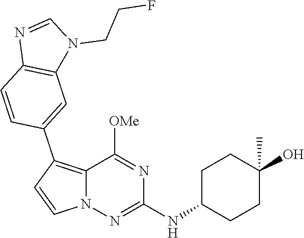 [SMILES: COc1nc(N[C@H]2CC[C@](C)(O)CC2)nn2ccc(-c3ccc4ncn(CCF)c4c3)c12] |
| 94 | 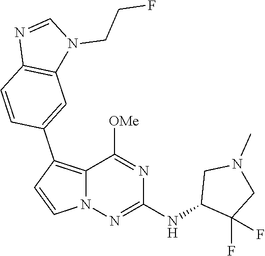 [SMILES: COc1nc(N[C@@H]2CN(C)CC2(F)F)nn2ccc(-c3ccc4ncn(CCF)c4c3)c12] |
| 95 | 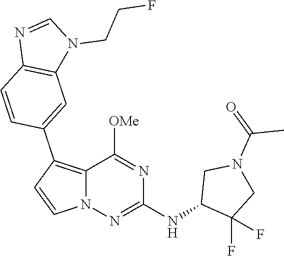 [SMILES: COc1nc(N[C@@H]2CN(C(C)=O)CC2(F)F)nn2ccc(-c3ccc4ncn(CCF)c4c3)c12] |
| 96 | 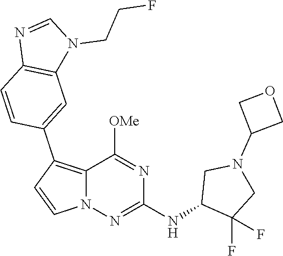 [SMILES: COc1nc(N[C@@H]2CN(C3COC3)CC2(F)F)nn2ccc(-c3ccc4ncn(CCF)c4c3)c12] |
| 97 | 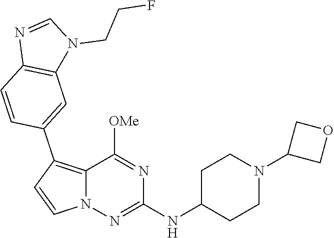 [SMILES: COc1nc(NC2CCN(C3COC3)CC2)nn2ccc(-c3ccc4ncn(CCF)c4c3)c12] |
| 98 | 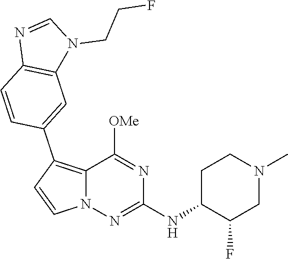 [SMILES: COc1nc(N[C@@H]2CCN(C)C[C@@H]2F)nn2ccc(-c3ccc4ncn(CCF)c4c3)c12] |
| 99 | 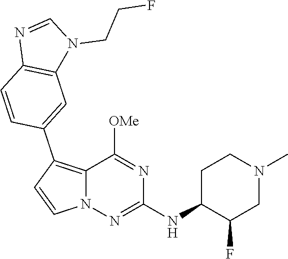 [SMILES: COc1nc(N[C@H]2CCN(C)C[C@H]2F)nn2ccc(-c3ccc4ncn(CCF)c4c3)c12] |
| 100 | 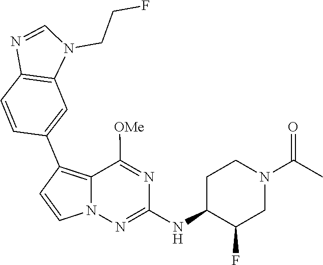 [SMILES: COc1nc(N[C@H]2CCN(C(C)=O)C[C@H]2F)nn2ccc(-c3ccc4ncn(CCF)c4c3)c12] |
| 101 | 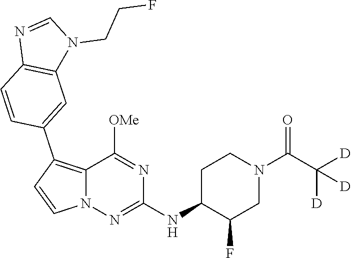 [SMILES: [2H]C([2H])([2H])C(=O)N1CC[C@H](Nc2nc(OC)c3c(-c4ccc5ncn(CCF)c5c4)ccn3n2)[C@H](F)C1] |
| 102 | 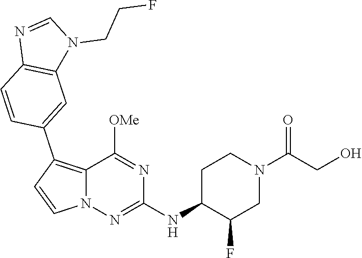 [SMILES: COc1nc(N[C@H]2CCN(C(=O)CO)C[C@H]2F)nn2ccc(-c3ccc4ncn(CCF)c4c3)c12] |
| 103 | 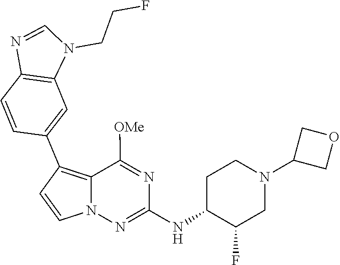 [SMILES: COc1nc(N[C@@H]2CCN(C3COC3)C[C@@H]2F)nn2ccc(-c3ccc4ncn(CCF)c4c3)c12] |
| 104 | 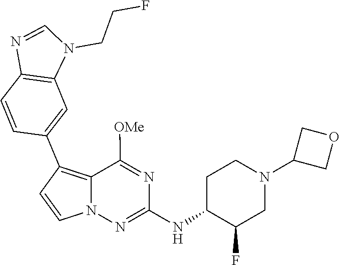 [SMILES: COc1nc(N[C@@H]2CCN(C3COC3)C[C@H]2F)nn2ccc(-c3ccc4ncn(CCF)c4c3)c12] |
| 105 | 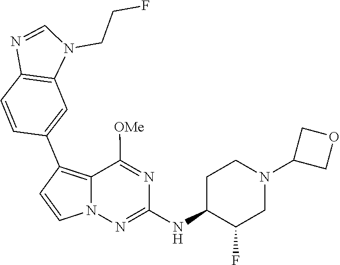 [SMILES: COc1nc(N[C@H]2CCN(C3COC3)C[C@@H]2F)nn2ccc(-c3ccc4ncn(CCF)c4c3)c12] |
| 106 | 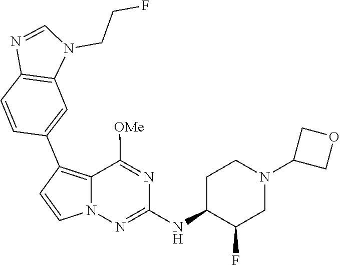 [SMILES: COc1nc(N[C@H]2CCN(C3COC3)C[C@H]2F)nn2ccc(-c3ccc4ncn(CCF)c4c3)c12] |
| 107 | 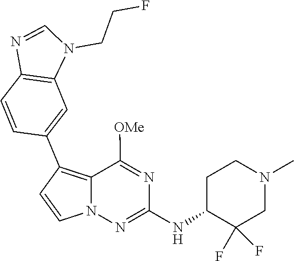 [SMILES: COc1nc(N[C@@H]2CCN(C)CC2(F)F)nn2ccc(-c3ccc4ncn(CCF)c4c3)c12] |
| 108 | 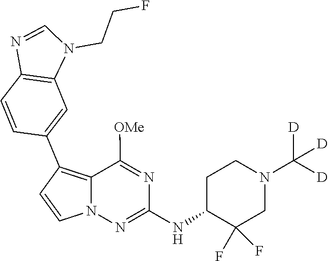 [SMILES: [2H]C([2H])([2H])N1CC[C@@H](Nc2nc(OC)c3c(-c4ccc5ncn(CCF)c5c4)ccn3n2)C(F)(F)C1] |
| 109 | 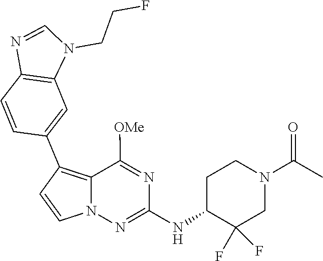 [SMILES: COc1nc(N[C@@H]2CCN(C(C)=O)CC2(F)F)nn2ccc(-c3ccc4ncn(CCF)c4c3)c12] |
| 110 | 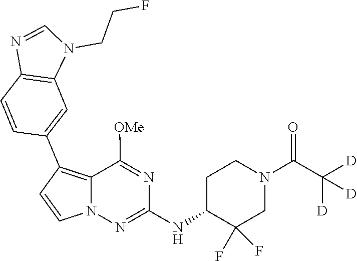 [SMILES: [2H]C([2H])([2H])C(=O)N1CC[C@@H](Nc2nc(OC)c3c(-c4ccc5ncn(CCF)c5c4)ccn3n2)C(F)(F)C1] |
| 111 | 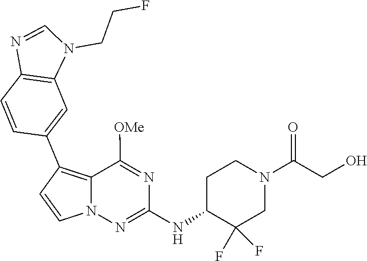 [SMILES: COc1nc(N[C@@H]2CCN(C(=O)CO)CC2(F)F)nn2ccc(-c3ccc4ncn(CCF)c4c3)c12] |
| 112 | 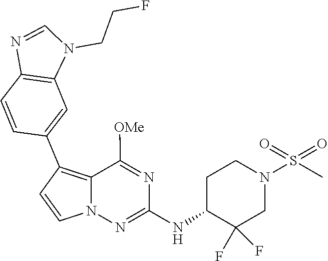 [SMILES: COc1nc(N[C@@H]2CCN(S(C)(=O)=O)CC2(F)F)nn2ccc(-c3ccc4ncn(CCF)c4c3)c12] |
| 113 | 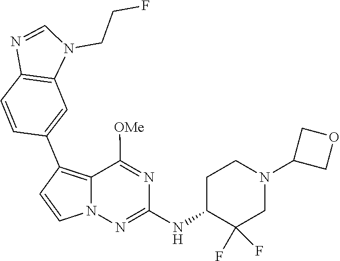 [SMILES: COc1nc(N[C@@H]2CCN(C3COC3)CC2(F)F)nn2ccc(-c3ccc4ncn(CCF)c4c3)c12] |
| 114 | 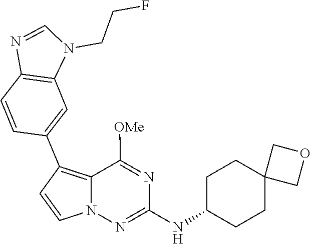 |
| 115 | 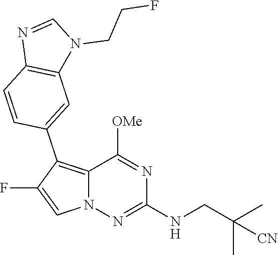 [SMILES: COc1nc(NCC(C)(C)C#N)nn2cc(F)c(-c3ccc4ncn(CCF)c4c3)c12] |
| 116 | 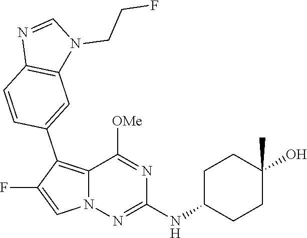 [SMILES: COc1nc(N[C@H]2CC[C@@](C)(O)CC2)nn2cc(F)c(-c3ccc4ncn(CCF)c4c3)c12] |
| 117 | 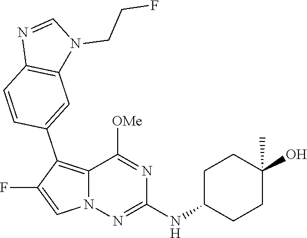 [SMILES: COc1nc(N[C@H]2CC[C@](C)(O)CC2)nn2cc(F)c(-c3ccc4ncn(CCF)c4c3)c12] |
| 118 | 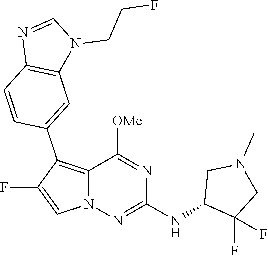 [SMILES: COc1nc(N[C@@H]2CN(C)CC2(F)F)nn2cc(F)c(-c3ccc4ncn(CCF)c4c3)c12] |
| 119 | 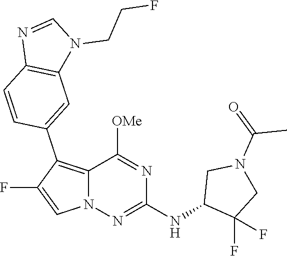 [SMILES: COc1nc(N[C@@H]2CN(C(C)=O)CC2(F)F)nn2cc(F)c(-c3ccc4ncn(CCF)c4c3)c12] |
| 120 | 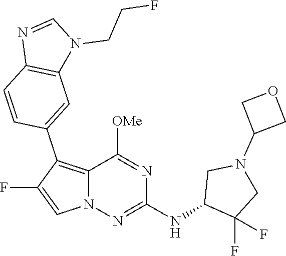 [SMILES: COc1nc(N[C@@H]2CN(C3COC3)CC2(F)F)nn2cc(F)c(-c3ccc4ncn(CCF)c4c3)c12] |
| 121 | 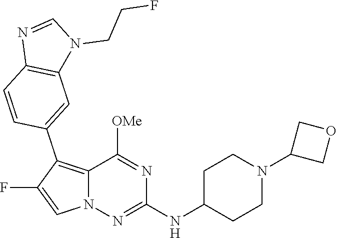 [SMILES: COc1nc(NC2CCN(C3COC3)CC2)nn2cc(F)c(-c3ccc4ncn(CCF)c4c3)c12] |
| 122 | 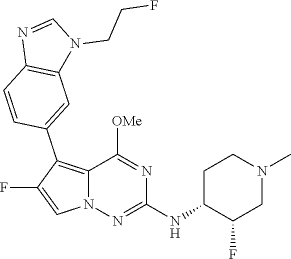 [SMILES: COc1nc(N[C@@H]2CCN(C)C[C@@H]2F)nn2cc(F)c(-c3ccc4ncn(CCF)c4c3)c12] |
| 123 | 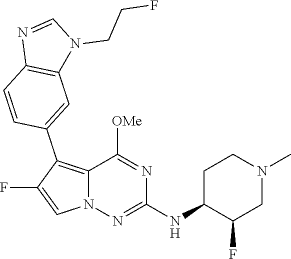 [SMILES: COc1nc(N[C@H]2CCN(C)C[C@H]2F)nn2cc(F)c(-c3ccc4ncn(CCF)c4c3)c12] |
| 124 | 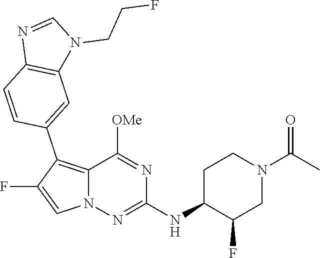 [SMILES: COc1nc(N[C@H]2CCN(C(C)=O)C[C@H]2F)nn2cc(F)c(-c3ccc4ncn(CCF)c4c3)c12] |
| 125 | 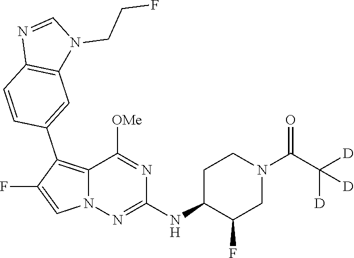 [SMILES: [2H]C([2H])([2H])C(=O)N1CC[C@H](Nc2nc(OC)c3c(-c4ccc5ncn(CCF)c5c4)c(F)cn3n2)[C@H](F)C1] |
| 126 | 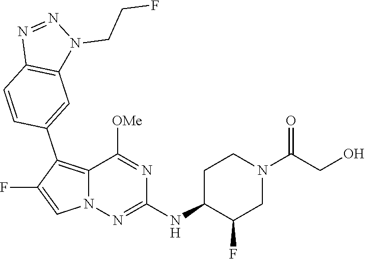 [SMILES: COc1nc(N[C@H]2CCN(C(=O)CO)C[C@H]2F)nn2cc(F)c(-c3ccc4nnn(CCF)c4c3)c12] |
| 127 | 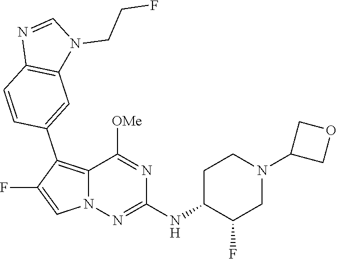 [SMILES: COc1nc(N[C@@H]2CCN(C3COC3)C[C@@H]2F)nn2cc(F)c(-c3ccc4ncn(CCF)c4c3)c12] |
| 128 | 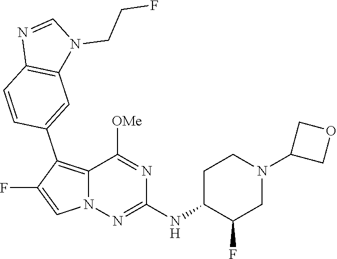 [SMILES: COc1nc(N[C@@H]2CCN(C3COC3)C[C@H]2F)nn2cc(F)c(-c3ccc4ncn(CCF)c4c3)c12] |
| 129 | 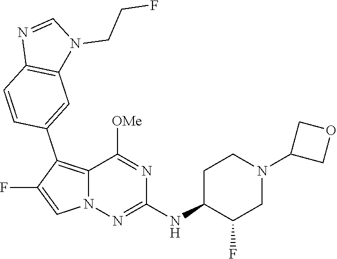 [SMILES: COc1nc(N[C@H]2CCN(C3COC3)C[C@@H]2F)nn2cc(F)c(-c3ccc4ncn(CCF)c4c3)c12] |
| 130 | 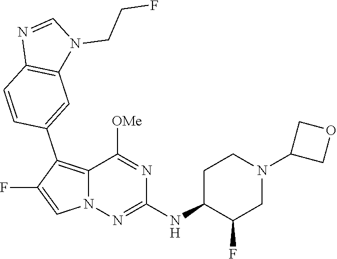 [SMILES: COc1nc(N[C@H]2CCN(C3COC3)C[C@H]2F)nn2cc(F)c(-c3ccc4ncn(CCF)c4c3)c12] |
| 131 | 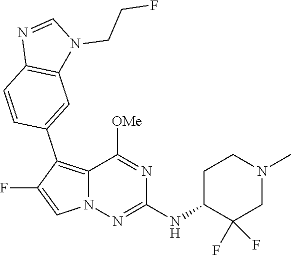 [SMILES: COc1nc(N[C@@H]2CCN(C)CC2(F)F)nn2cc(F)c(-c3ccc4ncn(CCF)c4c3)c12] |
| 132 | 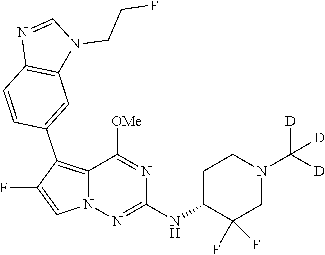 [SMILES: [2H]C([2H])([2H])N1CC[C@@H](Nc2nc(OC)c3c(-c4ccc5ncn(CCF)c5c4)c(F)cn3n2)C(F)(F)C1] |
| 133 | 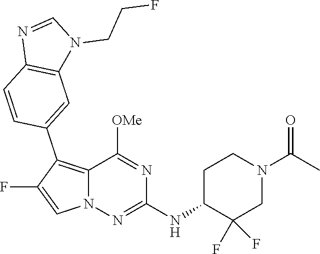 [SMILES: COc1nc(N[C@@H]2CCN(C(C)=O)CC2(F)F)nn2cc(F)c(-c3ccc4ncn(CCF)c4c3)c12] |
| 134 | 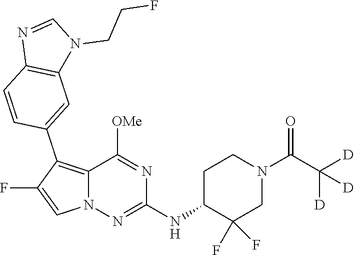 [SMILES: [2H]C([2H])([2H])C(=O)N1CC[C@@H](Nc2nc(OC)c3c(-c4ccc5ncn(CCF)c5c4)c(F)cn3n2)C(F)(F)C1] |
| 135 | 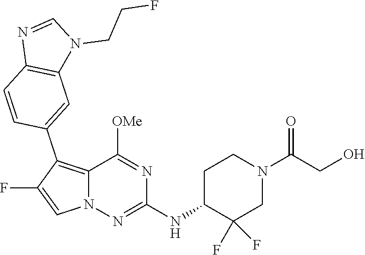 [SMILES: COc1nc(N[C@@H]2CCN(C(=O)CO)CC2(F)F)nn2cc(F)c(-c3ccc4ncn(CCF)c4c3)c12] |
| 136 | 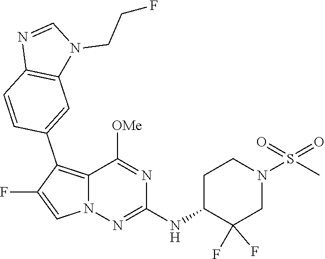 [SMILES: COc1nc(N[C@@H]2CCN(S(C)(=O)=O)CC2(F)F)nn2cc(F)c(-c3ccc4ncn(CCF)c4c3)c12] |
| 137 | 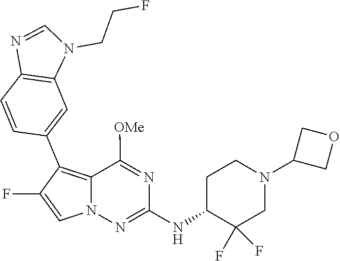 [SMILES: COc1nc(N[C@@H]2CCN(C3COC3)CC2(F)F)nn2cc(F)c(-c3ccc4ncn(CCF)c4c3)c12] |
| 138 | 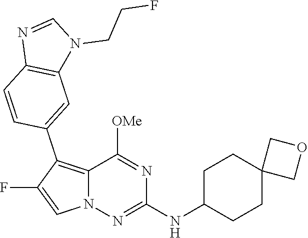 [SMILES: COc1nc(NC2CCC3(CC2)COC3)nn2cc(F)c(-c3ccc4ncn(CCF)c4c3)c12] |
| 139 | 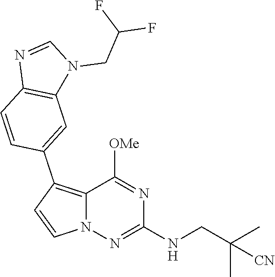 [SMILES: COc1nc(NCC(C)(C)C#N)nn2ccc(-c3ccc4ncn(CC(F)F)c4c3)c12] |
| 140 | 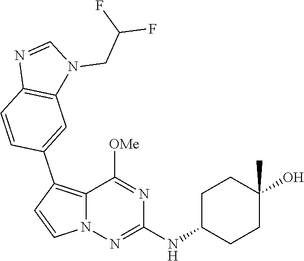 [SMILES: COc1nc(N[C@H]2CC[C@@](C)(O)CC2)nn2ccc(-c3ccc4ncn(CC(F)F)c4c3)c12] |
| 141 | 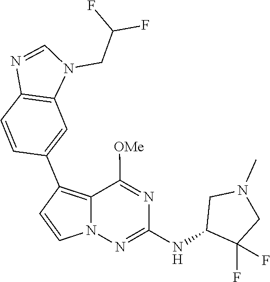 [SMILES: COc1nc(N[C@@H]2CN(C)CC2(F)F)nn2ccc(-c3ccc4ncn(CC(F)F)c4c3)c12] |
| 142 | 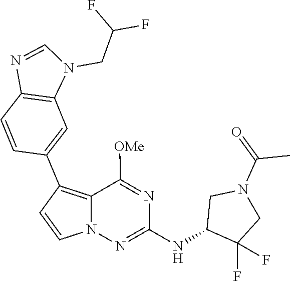 [SMILES: COc1nc(N[C@@H]2CN(C(C)=O)CC2(F)F)nn2ccc(-c3ccc4ncn(CC(F)F)c4c3)c12] |
| 143 | 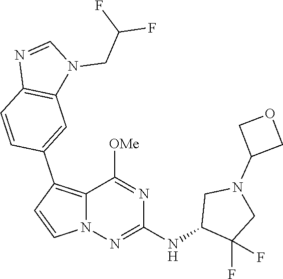 [SMILES: COc1nc(N[C@@H]2CN(C3COC3)CC2(F)F)nn2ccc(-c3ccc4ncn(CC(F)F)c4c3)c12] |
| 144 | 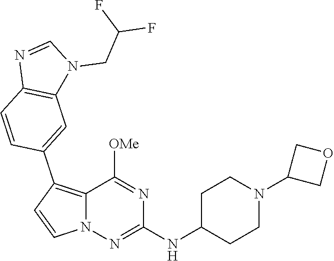 [SMILES: COc1nc(NC2CCN(C3COC3)CC2)nn2ccc(-c3ccc4ncn(CC(F)F)c4c3)c12] |
| 145 | 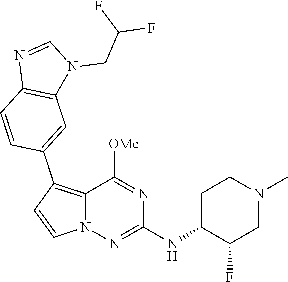 [SMILES: COc1nc(N[C@@H]2CCN(C)C[C@@H]2F)nn2ccc(-c3ccc4ncn(CC(F)F)c4c3)c12] |
| 146 | 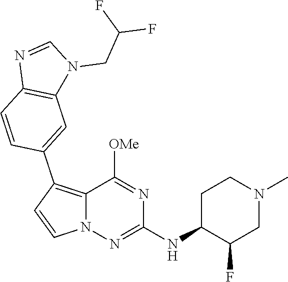 [SMILES: COc1nc(N[C@H]2CCN(C)C[C@H]2F)nn2ccc(-c3ccc4ncn(CC(F)F)c4c3)c12] |
| 147 | 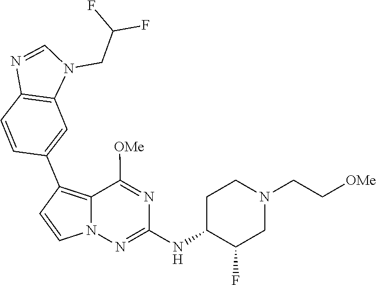 [SMILES: COCCN1CC[C@@H](Nc2nc(OC)c3c(-c4ccc5ncn(CC(F)F)c5c4)ccn3n2)[C@@H](F)C1] |
| 148 | 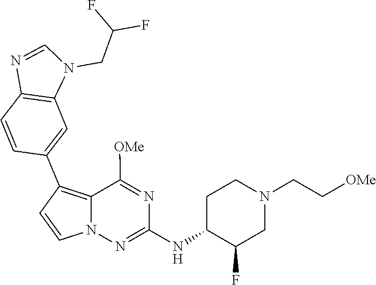 [SMILES: COCCN1CC[C@@H](Nc2nc(OC)c3c(-c4ccc5ncn(CC(F)F)c5c4)ccn3n2)[C@H](F)C1] |
| 149 | 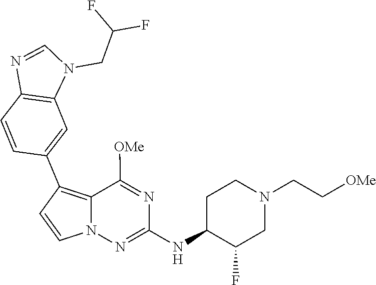 [SMILES: COCCN1CC[C@H](Nc2nc(OC)c3c(-c4ccc5ncn(CC(F)F)c5c4)ccn3n2)[C@@H](F)C1] |
| 150 | 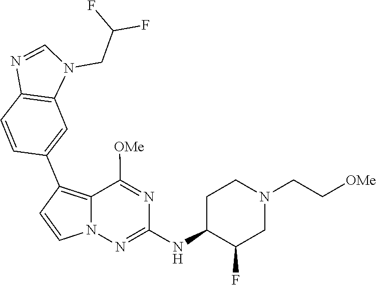 [SMILES: COCCN1CC[C@H](Nc2nc(OC)c3c(-c4ccc5ncn(CC(F)F)c5c4)ccn3n2)[C@H](F)C1] |
| 151 | 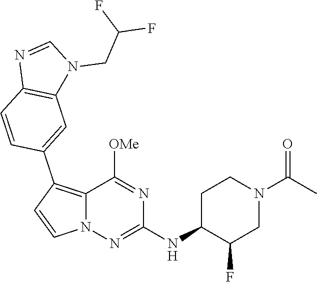 [SMILES: COc1nc(N[C@H]2CCN(C(C)=O)C[C@H]2F)nn2ccc(-c3ccc4ncn(CC(F)F)c4c3)c12] |
| 152 | 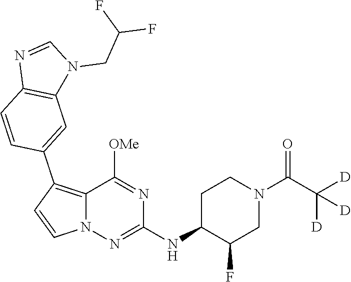 [SMILES: [2H]C([2H])([2H])C(=O)N1CC[C@H](Nc2nc(OC)c3c(-c4ccc5ncn(CC(F)F)c5c4)ccn3n2)[C@H](F)C1] |
| 153 | 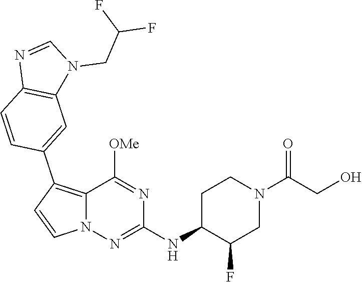 [SMILES: COc1nc(N[C@H]2CCN(C(=O)CO)C[C@H]2F)nn2ccc(-c3ccc4ncn(CC(F)F)c4c3)c12] |
| 154 | 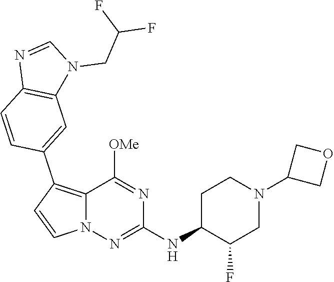 [SMILES: COc1nc(N[C@H]2CCN(C3COC3)C[C@@H]2F)nn2ccc(-c3ccc4ncn(CC(F)F)c4c3)c12] |
| 155 | 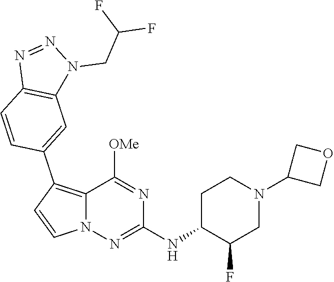 [SMILES: COc1nc(N[C@@H]2CCN(C3COC3)C[C@H]2F)nn2ccc(-c3ccc4nnn(CC(F)F)c4c3)c12] |
| 156 | 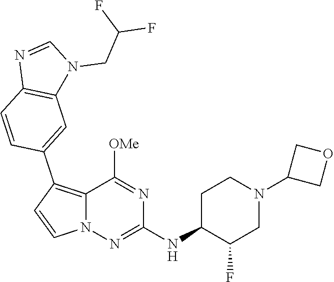 [SMILES: COc1nc(N[C@H]2CCN(C3COC3)C[C@@H]2F)nn2ccc(-c3ccc4ncn(CC(F)F)c4c3)c12] |
| 157 | 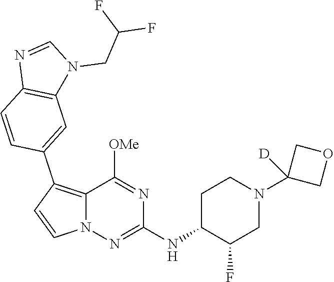 [SMILES: [2H]C1(N2CC[C@@H](Nc3nc(OC)c4c(-c5ccc6ncn(CC(F)F)c6c5)ccn4n3)[C@@H](F)C2)COC1] |
| 158 | 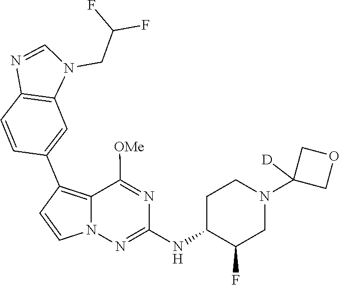 [SMILES: [2H]C1(N2CC[C@@H](Nc3nc(OC)c4c(-c5ccc6ncn(CC(F)F)c6c5)ccn4n3)[C@H](F)C2)COC1] |
| 159 | 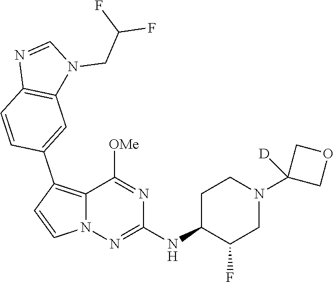 [SMILES: [2H]C1(N2CC[C@H](Nc3nc(OC)c4c(-c5ccc6ncn(CC(F)F)c6c5)ccn4n3)[C@@H](F)C2)COC1] |
| 160 | 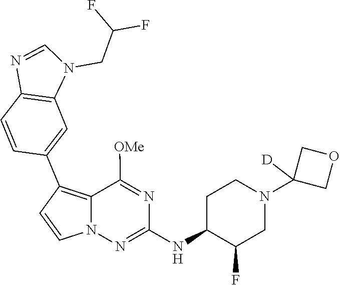 [SMILES: [2H]C1(N2CC[C@H](Nc3nc(OC)c4c(-c5ccc6ncn(CC(F)F)c6c5)ccn4n3)[C@H](F)C2)COC1] |
| 161 | 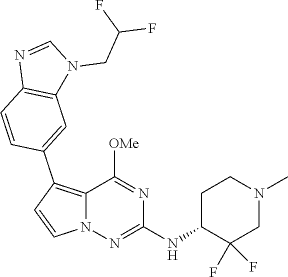 [SMILES: COc1nc(N[C@@H]2CCN(C)CC2(F)F)nn2ccc(-c3ccc4ncn(CC(F)F)c4c3)c12] |
| 162 | 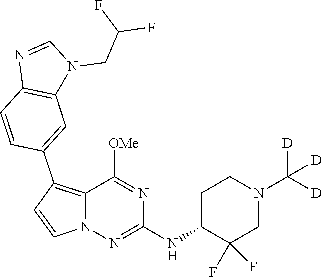 [SMILES: [2H]C([2H])([2H])N1CC[C@@H](Nc2nc(OC)c3c(-c4ccc5ncn(CC(F)F)c5c4)ccn3n2)C(F)(F)C1] |
| 163 | 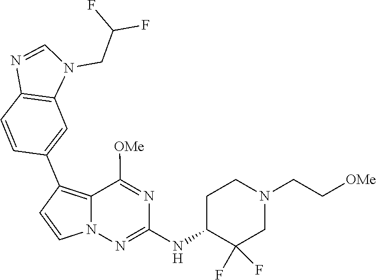 [SMILES: COCCN1CC[C@@H](Nc2nc(OC)c3c(-c4ccc5ncn(CC(F)F)c5c4)ccn3n2)C(F)(F)C1] |
| 164 | 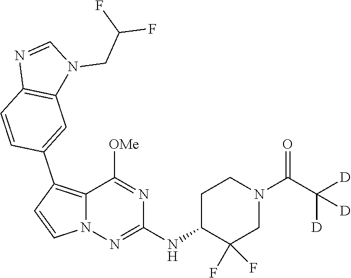 [SMILES: [2H]C([2H])([2H])C(=O)N1CC[C@@H](Nc2nc(OC)c3c(-c4ccc5ncn(CC(F)F)c5c4)ccn3n2)C(F)(F)C1] |
| 165 | 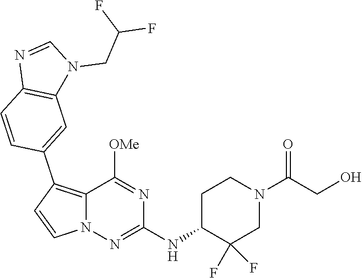 [SMILES: COc1nc(N[C@@H]2CCN(C(=O)CO)CC2(F)F)nn2ccc(-c3ccc4ncn(CC(F)F)c4c3)c12] |
| 166 | 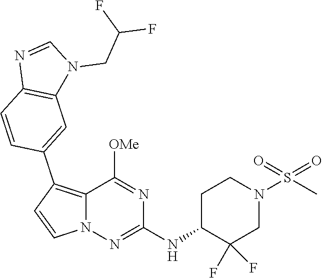 [SMILES: COc1nc(N[C@@H]2CCN(S(C)(=O)=O)CC2(F)F)nn2ccc(-c3ccc4ncn(CC(F)F)c4c3)c12] |
| 167 | 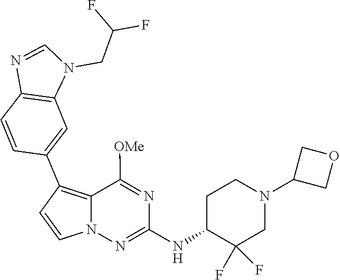 [SMILES: COc1nc(N[C@@H]2CCN(C3COC3)CC2(F)F)nn2ccc(-c3ccc4ncn(CC(F)F)c4c3)c12] |
| 168 | 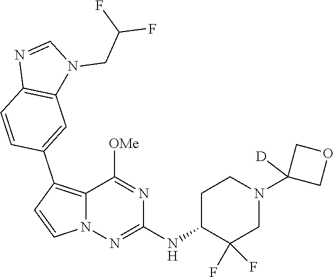 [SMILES: [2H]C1(N2CC[C@@H](Nc3nc(OC)c4c(-c5ccc6ncn(CC(F)F)c6c5)ccn4n3)C(F)(F)C2)COC1] |
| 169 | 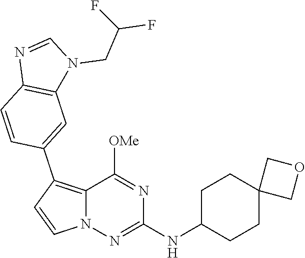 [SMILES: COc1nc(NC2CCC3(CC2)COC3)nn2ccc(-c3ccc4ncn(CC(F)F)c4c3)c12] |
| 170 | 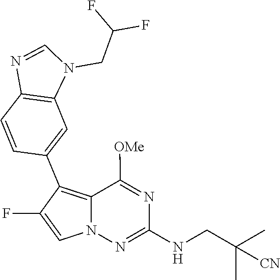 [SMILES: COc1nc(NCC(C)(C)C#N)nn2cc(F)c(-c3ccc4ncn(CC(F)F)c4c3)c12] |
| 171 | 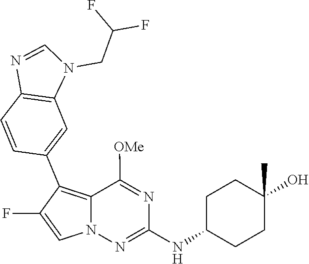 [SMILES: COc1nc(N[C@H]2CC[C@@](C)(O)CC2)nn2cc(F)c(-c3ccc4ncn(CC(F)F)c4c3)c12] |
| 172 | 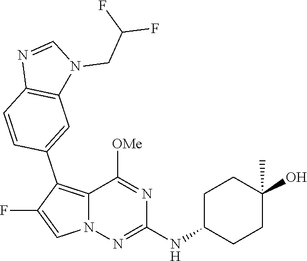 [SMILES: COc1nc(N[C@H]2CC[C@](C)(O)CC2)nn2cc(F)c(-c3ccc4ncn(CC(F)F)c4c3)c12] |
| 173 | 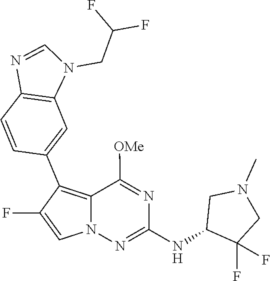 [SMILES: COc1nc(N[C@@H]2CN(C)CC2(F)F)nn2cc(F)c(-c3ccc4ncn(CC(F)F)c4c3)c12] |
| 174 | 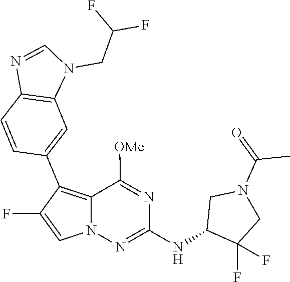 [SMILES: COc1nc(N[C@@H]2CN(C(C)=O)CC2(F)F)nn2cc(F)c(-c3ccc4ncn(CC(F)F)c4c3)c12] |
| 175 | 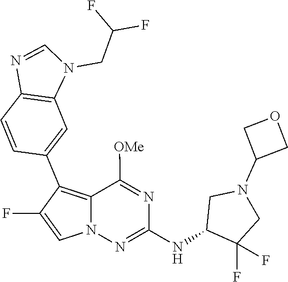 [SMILES: COc1nc(N[C@@H]2CN(C3COC3)CC2(F)F)nn2cc(F)c(-c3ccc4ncn(CC(F)F)c4c3)c12] |
| 176 | 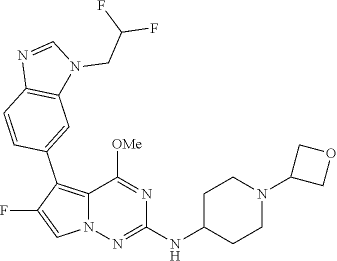 [SMILES: COc1nc(NC2CCN(C3COC3)CC2)nn2cc(F)c(-c3ccc4ncn(CC(F)F)c4c3)c12] |
| 177 | 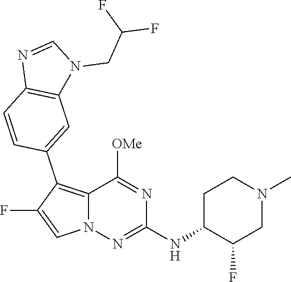 [SMILES: COc1nc(N[C@@H]2CCN(C)C[C@@H]2F)nn2cc(F)c(-c3ccc4ncn(CC(F)F)c4c3)c12] |
| 178 | 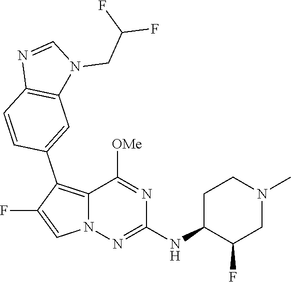 [SMILES: COc1nc(N[C@H]2CCN(C)C[C@H]2F)nn2cc(F)c(-c3ccc4ncn(CC(F)F)c4c3)c12] |
| 179 | 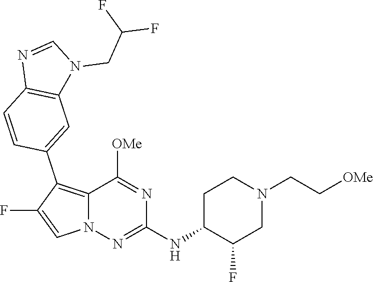 [SMILES: COCCN1CC[C@@H](Nc2nc(OC)c3c(-c4ccc5ncn(CC(F)F)c5c4)c(F)cn3n2)[C@@H](F)C1] |
| 180 | 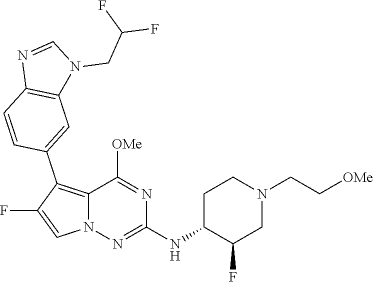 [SMILES: COCCN1CC[C@@H](Nc2nc(OC)c3c(-c4ccc5ncn(CC(F)F)c5c4)c(F)cn3n2)[C@H](F)C1] |
| 181 | 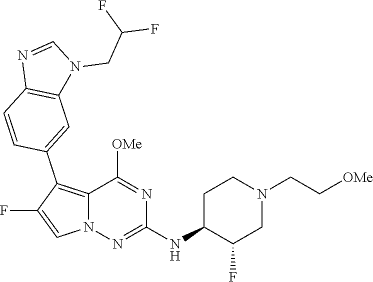 [SMILES: COCCN1CC[C@H](Nc2nc(OC)c3c(-c4ccc5ncn(CC(F)F)c5c4)c(F)cn3n2)[C@@H](F)C1] |
| 182 | 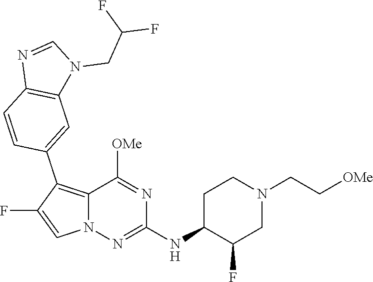 [SMILES: COCCN1CC[C@H](Nc2nc(OC)c3c(-c4ccc5ncn(CC(F)F)c5c4)c(F)cn3n2)[C@H](F)C1] |
| 183 | 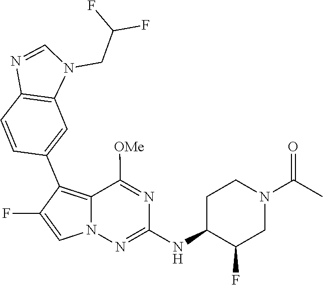 [SMILES: COc1nc(N[C@H]2CCN(C(C)=O)C[C@H]2F)nn2cc(F)c(-c3ccc4ncn(CC(F)F)c4c3)c12] |
| 184 | 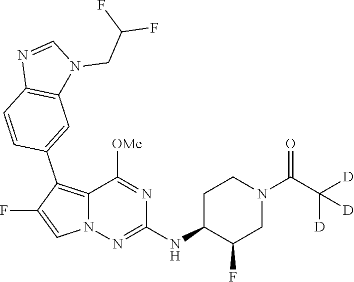 [SMILES: [2H]C([2H])([2H])C(=O)N1CC[C@H](Nc2nc(OC)c3c(-c4ccc5ncn(CC(F)F)c5c4)c(F)cn3n2)[C@H](F)C1] |
| 185 | 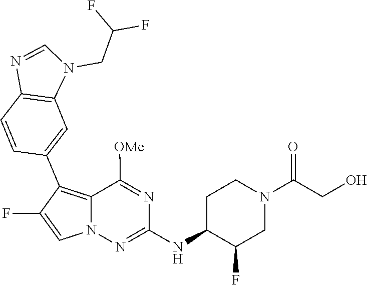 [SMILES: COc1nc(N[C@H]2CCN(C(=O)CO)C[C@H]2F)nn2cc(F)c(-c3ccc4ncn(CC(F)F)c4c3)c12] |
| 186 | 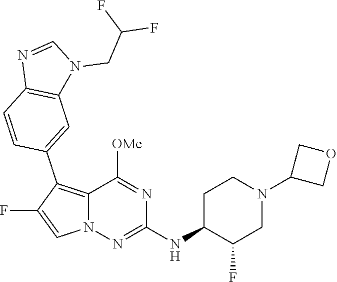 [SMILES: COc1nc(N[C@H]2CCN(C3COC3)C[C@@H]2F)nn2cc(F)c(-c3ccc4ncn(CC(F)F)c4c3)c12] |
| 187 | 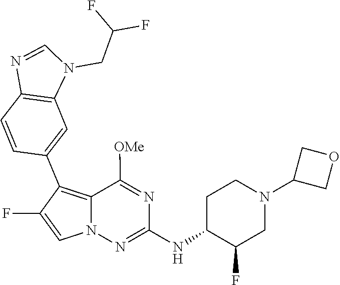 [SMILES: COc1nc(N[C@@H]2CCN(C3COC3)C[C@H]2F)nn2cc(F)c(-c3ccc4ncn(CC(F)F)c4c3)c12] |
| 188 | 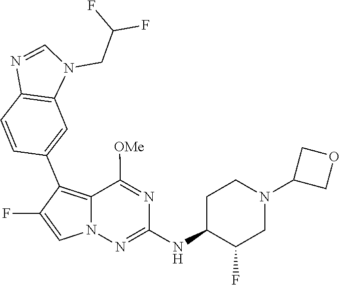 [SMILES: COc1nc(N[C@H]2CCN(C3COC3)C[C@@H]2F)nn2cc(F)c(-c3ccc4ncn(CC(F)F)c4c3)c12] |
| 189 | 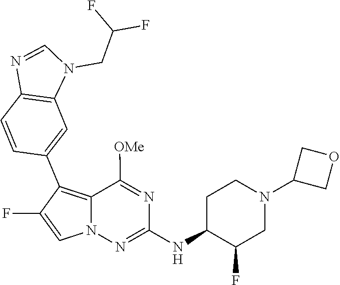 [SMILES: COc1nc(N[C@H]2CCN(C3COC3)C[C@H]2F)nn2cc(F)c(-c3ccc4ncn(CC(F)F)c4c3)c12] |
| 190 | 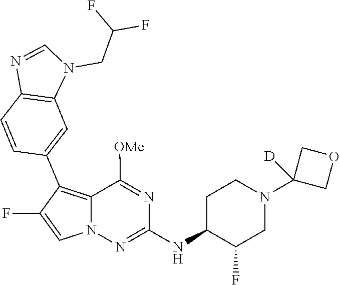 [SMILES: [2H]C1(N2CC[C@H](Nc3nc(OC)c4c(-c5ccc6ncn(CC(F)F)c6c5)c(F)cn4n3)[C@@H](F)C2)COC1] |
| 191 | 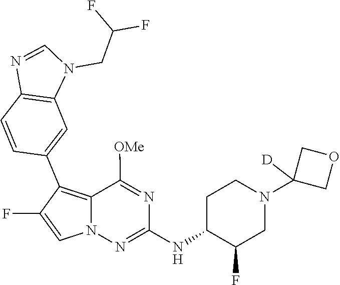 [SMILES: [2H]C1(N2CC[C@@H](Nc3nc(OC)c4c(-c5ccc6ncn(CC(F)F)c6c5)c(F)cn4n3)[C@H](F)C2)COC1] |
| 192 | 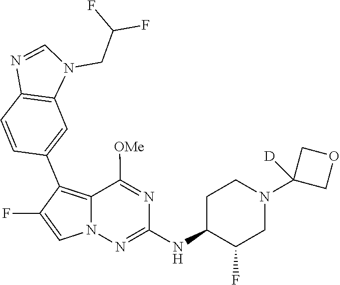 [SMILES: [2H]C1(N2CC[C@H](Nc3nc(OC)c4c(-c5ccc6ncn(CC(F)F)c6c5)c(F)cn4n3)[C@@H](F)C2)COC1] |
| 193 | 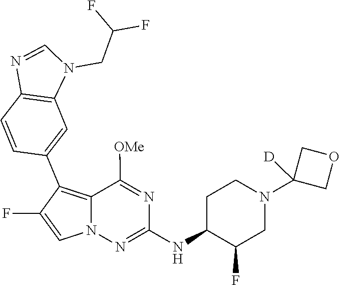 [SMILES: [2H]C1(N2CC[C@H](Nc3nc(OC)c4c(-c5ccc6ncn(CC(F)F)c6c5)c(F)cn4n3)[C@H](F)C2)COC1] |
| 194 | 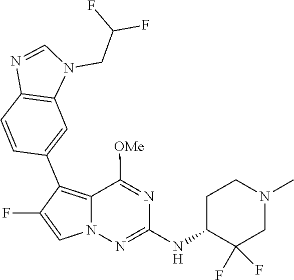 [SMILES: COc1nc(N[C@@H]2CCN(C)CC2(F)F)nn2cc(F)c(-c3ccc4ncn(CC(F)F)c4c3)c12] |
| 195 | 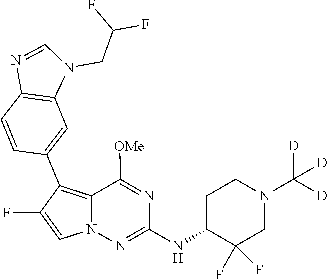 [SMILES: [2H]C([2H])([2H])N1CC[C@@H](Nc2nc(OC)c3c(-c4ccc5ncn(CC(F)F)c5c4)c(F)cn3n2)C(F)(F)C1] |
| 196 | 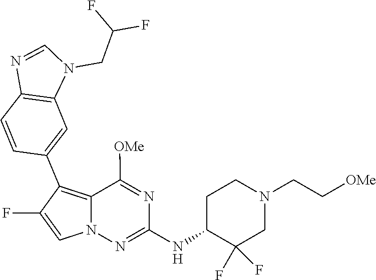 [SMILES: COCCN1CC[C@@H](Nc2nc(OC)c3c(-c4ccc5ncn(CC(F)F)c5c4)c(F)cn3n2)C(F)(F)C1] |
| 197 | 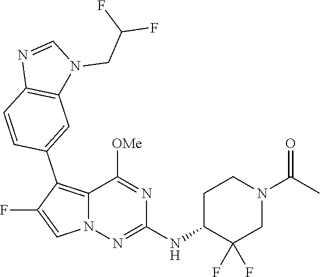 [SMILES: COc1nc(N[C@@H]2CCN(C(C)=O)CC2(F)F)nn2cc(F)c(-c3ccc4ncn(CC(F)F)c4c3)c12] |
| 198 | 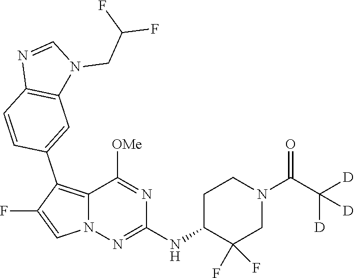 [SMILES: [2H]C([2H])([2H])C(=O)N1CC[C@@H](Nc2nc(OC)c3c(-c4ccc5ncn(CC(F)F)c5c4)c(F)cn3n2)C(F)(F)C1] |
| 199 | 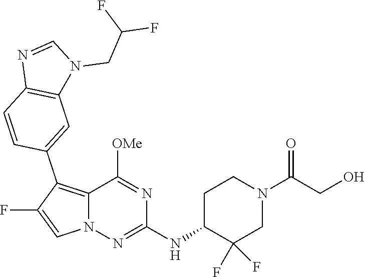 [SMILES: COc1nc(N[C@@H]2CCN(C(=O)CO)CC2(F)F)nn2cc(F)c(-c3ccc4ncn(CC(F)F)c4c3)c12] |
| 200 | 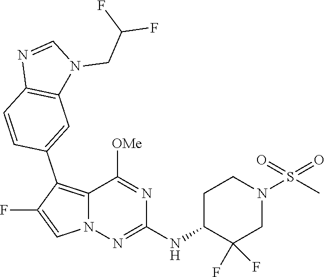 [SMILES: COc1nc(N[C@@H]2CCN(S(C)(=O)=O)CC2(F)F)nn2cc(F)c(-c3ccc4ncn(CC(F)F)c4c3)c12] |
| 201 | 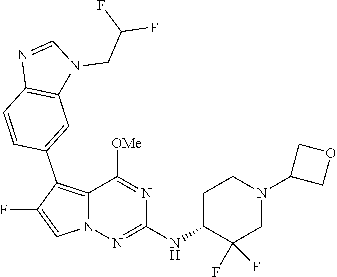 [SMILES: COc1nc(N[C@@H]2CCN(C3COC3)CC2(F)F)nn2cc(F)c(-c3ccc4ncn(CC(F)F)c4c3)c12] |
| 202 | 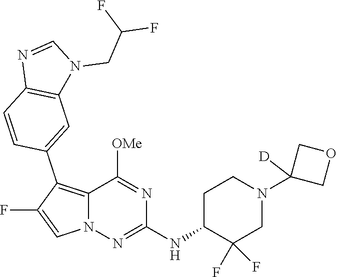 [SMILES: [2H]C1(N2CC[C@@H](Nc3nc(OC)c4c(-c5ccc6ncn(CC(F)F)c6c5)c(F)cn4n3)C(F)(F)C2)COC1] |
| 203 | 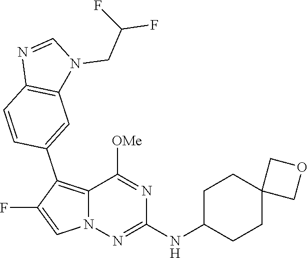 [SMILES: COc1nc(NC2CCC3(CC2)COC3)nn2cc(F)c(-c3ccc4ncn(CC(F)F)c4c3)c12] |
| 204 | 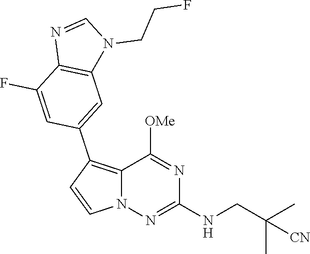 [SMILES: COc1nc(NCC(C)(C)C#N)nn2ccc(-c3cc(F)c4ncn(CCF)c4c3)c12] |
| 205 | 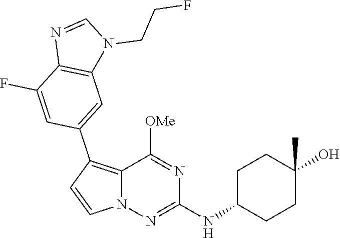 [SMILES: COc1nc(N[C@H]2CC[C@@](C)(O)CC2)nn2ccc(-c3cc(F)c4ncn(CCF)c4c3)c12] |
| 206 | 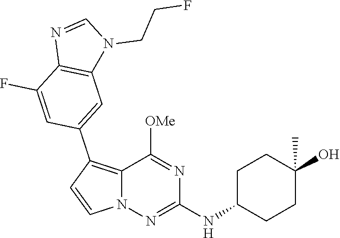 [SMILES: COc1nc(N[C@H]2CC[C@](C)(O)CC2)nn2ccc(-c3cc(F)c4ncn(CCF)c4c3)c12] |
| 207 | 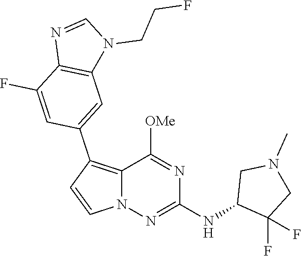 [SMILES: COc1nc(N[C@@H]2CN(C)CC2(F)F)nn2ccc(-c3cc(F)c4ncn(CCF)c4c3)c12] |
| 208 | 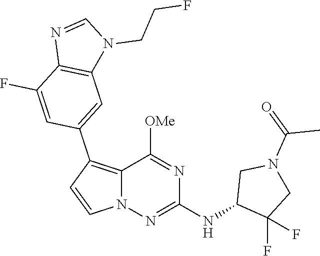 [SMILES: COc1nc(N[C@@H]2CN(C(C)=O)CC2(F)F)nn2ccc(-c3cc(F)c4ncn(CCF)c4c3)c12] |
| 209 | 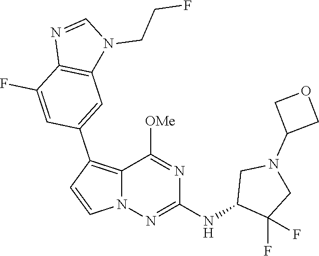 [SMILES: COc1nc(N[C@@H]2CN(C3COC3)CC2(F)F)nn2ccc(-c3cc(F)c4ncn(CCF)c4c3)c12] |
| 210 | 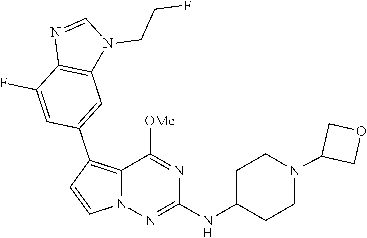 [SMILES: COc1nc(NC2CCN(C3COC3)CC2)nn2ccc(-c3cc(F)c4ncn(CCF)c4c3)c12] |
| 211 | 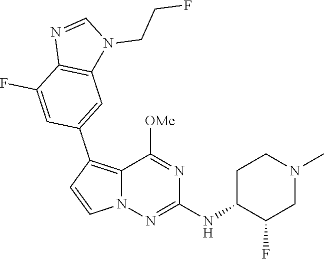 [SMILES: COc1nc(N[C@@H]2CCN(C)C[C@@H]2F)nn2ccc(-c3cc(F)c4ncn(CCF)c4c3)c12] |
| 212 | 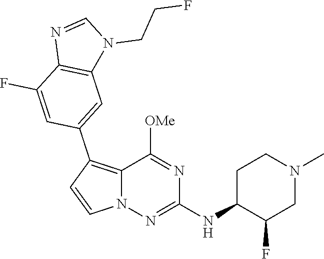 [SMILES: COc1nc(N[C@H]2CCN(C)C[C@H]2F)nn2ccc(-c3cc(F)c4ncn(CCF)c4c3)c12] |
| 213 | 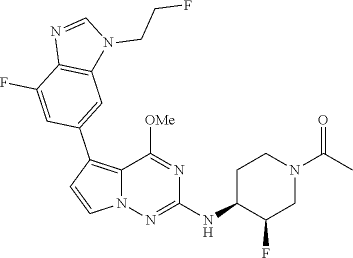 [SMILES: COc1nc(N[C@H]2CCN(C(C)=O)C[C@H]2F)nn2ccc(-c3cc(F)c4ncn(CCF)c4c3)c12] |
| 214 | 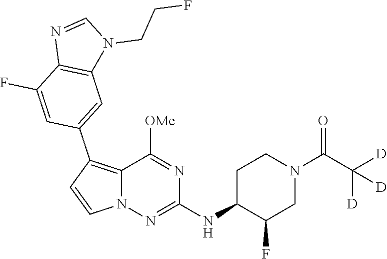 [SMILES: [2H]C([2H])([2H])C(=O)N1CC[C@H](Nc2nc(OC)c3c(-c4cc(F)c5ncn(CCF)c5c4)ccn3n2)[C@H](F)C1] |
| 215 | 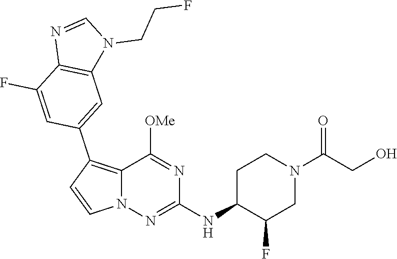 [SMILES: COc1nc(N[C@H]2CCN(C(=O)CO)C[C@H]2F)nn2ccc(-c3cc(F)c4ncn(CCF)c4c3)c12] |
| 216 | 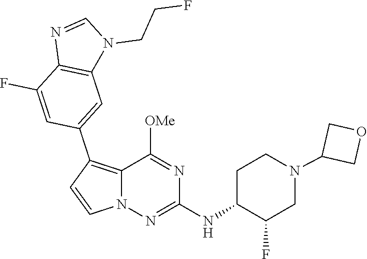 [SMILES: COc1nc(N[C@@H]2CCN(C3COC3)C[C@@H]2F)nn2ccc(-c3cc(F)c4ncn(CCF)c4c3)c12] |
| 217 | 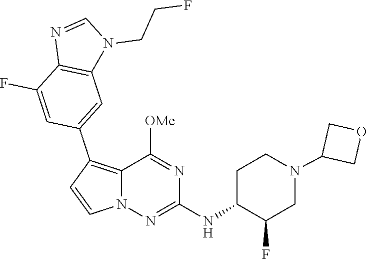 [SMILES: COc1nc(N[C@@H]2CCN(C3COC3)C[C@H]2F)nn2ccc(-c3cc(F)c4ncn(CCF)c4c3)c12] |
| 218 | 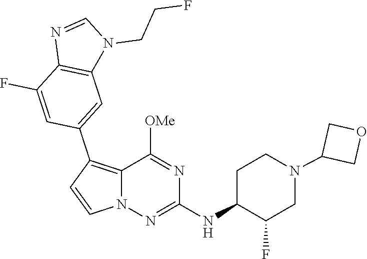 [SMILES: COc1nc(N[C@H]2CCN(C3COC3)C[C@@H]2F)nn2ccc(-c3cc(F)c4ncn(CCF)c4c3)c12] |
| 219 | 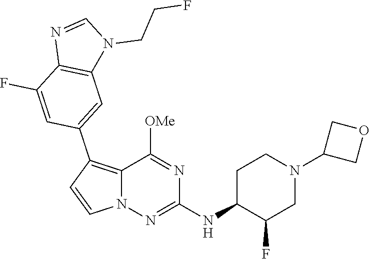 [SMILES: COc1nc(N[C@H]2CCN(C3COC3)C[C@H]2F)nn2ccc(-c3cc(F)c4ncn(CCF)c4c3)c12] |
| 220 | 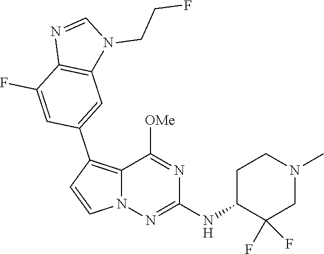 [SMILES: COc1nc(N[C@@H]2CCN(C)CC2(F)F)nn2ccc(-c3cc(F)c4ncn(CCF)c4c3)c12] |
| 221 | 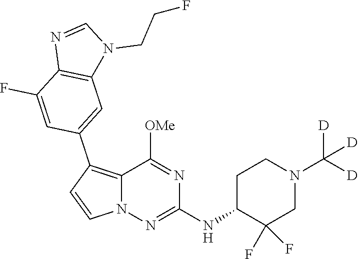 [SMILES: [2H]C([2H])([2H])N1CC[C@@H](Nc2nc(OC)c3c(-c4cc(F)c5ncn(CCF)c5c4)ccn3n2)C(F)(F)C1] |
| 222 | 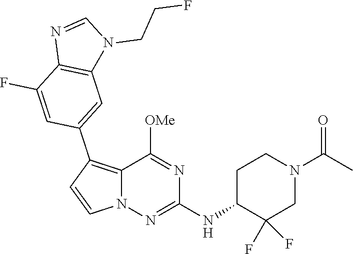 [SMILES: COc1nc(N[C@@H]2CCN(C(C)=O)CC2(F)F)nn2ccc(-c3cc(F)c4ncn(CCF)c4c3)c12] |
| 223 | 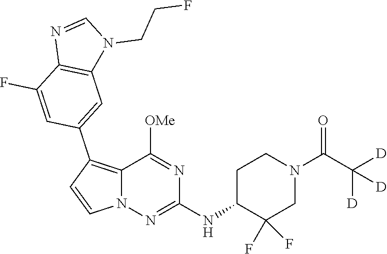 [SMILES: [2H]C([2H])([2H])C(=O)N1CC[C@@H](Nc2nc(OC)c3c(-c4cc(F)c5ncn(CCF)c5c4)ccn3n2)C(F)(F)C1] |
| 224 | 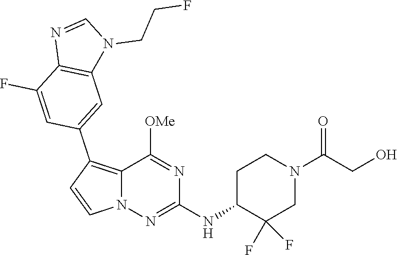 [SMILES: COc1nc(N[C@@H]2CCN(C(=O)CO)CC2(F)F)nn2ccc(-c3cc(F)c4ncn(CCF)c4c3)c12] |
| 225 | 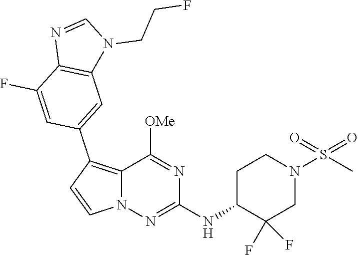 [SMILES: COc1nc(N[C@@H]2CCN(S(C)(=O)=O)CC2(F)F)nn2ccc(-c3cc(F)c4ncn(CCF)c4c3)c12] |
| 226 | 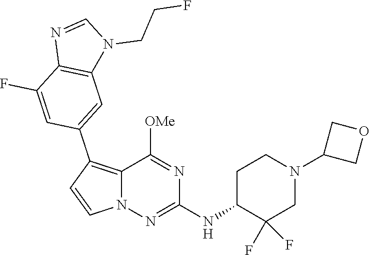 [SMILES: COc1nc(N[C@@H]2CCN(C3COC3)CC2(F)F)nn2ccc(-c3cc(F)c4ncn(CCF)c4c3)c12] |
| 227 | 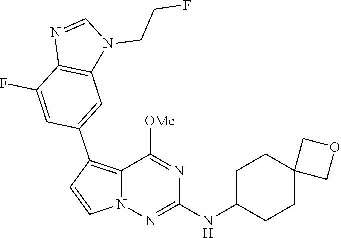 [SMILES: COc1nc(NC2CCC3(CC2)COC3)nn2ccc(-c3cc(F)c4ncn(CCF)c4c3)c12] |
| 228 | 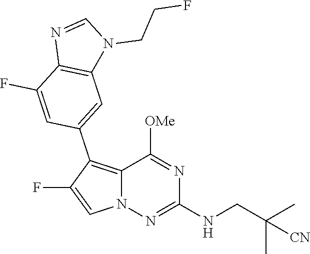 [SMILES: COc1nc(NCC(C)(C)C#N)nn2cc(F)c(-c3cc(F)c4ncn(CCF)c4c3)c12] |
| 229 | 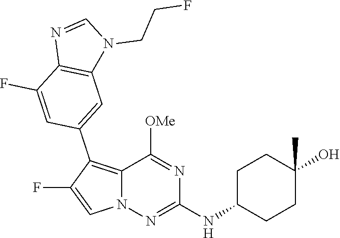 [SMILES: COc1nc(N[C@H]2CC[C@@](C)(O)CC2)nn2cc(F)c(-c3cc(F)c4ncn(CCF)c4c3)c12] |
| 230 | 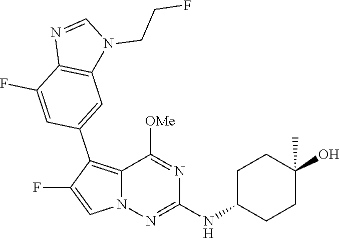 [SMILES: COc1nc(N[C@H]2CC[C@](C)(O)CC2)nn2cc(F)c(-c3cc(F)c4ncn(CCF)c4c3)c12] |
| 231 | 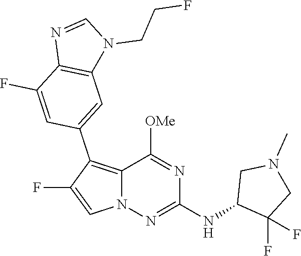 [SMILES: COc1nc(N[C@@H]2CN(C)CC2(F)F)nn2cc(F)c(-c3cc(F)c4ncn(CCF)c4c3)c12] |
| 232 | 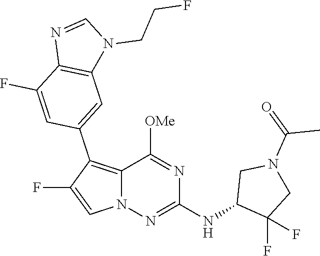 [SMILES: COc1nc(N[C@@H]2CN(C(C)=O)CC2(F)F)nn2cc(F)c(-c3cc(F)c4ncn(CCF)c4c3)c12] |
| 233 | 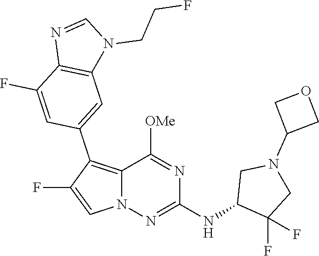 [SMILES: COc1nc(N[C@@H]2CN(C3COC3)CC2(F)F)nn2cc(F)c(-c3cc(F)c4ncn(CCF)c4c3)c12] |
| 234 | 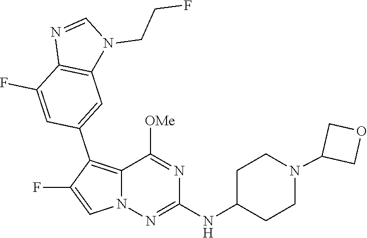 [SMILES: COc1nc(NC2CCN(C3COC3)CC2)nn2cc(F)c(-c3cc(F)c4ncn(CCF)c4c3)c12] |
| 235 | 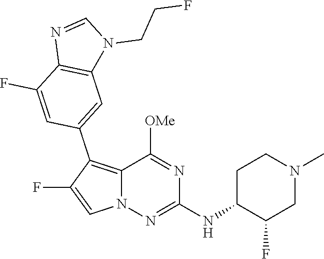 [SMILES: COc1nc(N[C@@H]2CCN(C)C[C@@H]2F)nn2cc(F)c(-c3cc(F)c4ncn(CCF)c4c3)c12] |
| 236 | 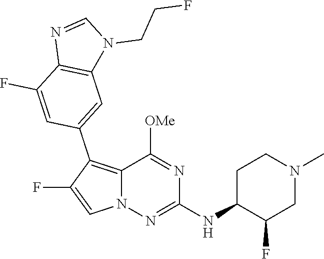 [SMILES: COc1nc(N[C@H]2CCN(C)C[C@H]2F)nn2cc(F)c(-c3cc(F)c4ncn(CCF)c4c3)c12] |
| 237 | 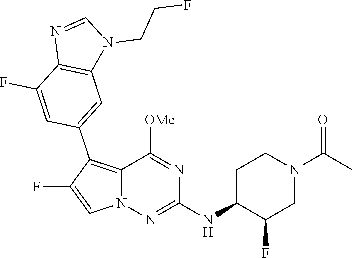 [SMILES: COc1nc(N[C@H]2CCN(C(C)=O)C[C@H]2F)nn2cc(F)c(-c3cc(F)c4ncn(CCF)c4c3)c12] |
| 238 | 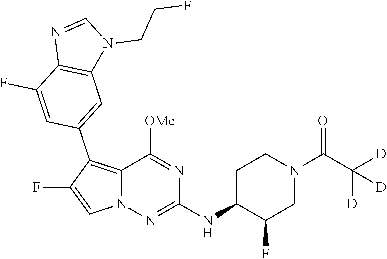 [SMILES: [2H]C([2H])([2H])C(=O)N1CC[C@H](Nc2nc(OC)c3c(-c4cc(F)c5ncn(CCF)c5c4)c(F)cn3n2)[C@H](F)C1] |
| 239 | 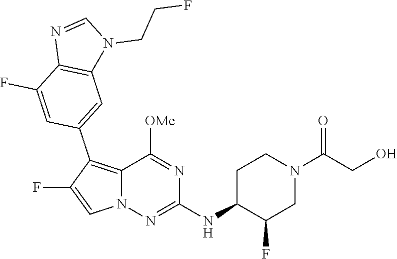 [SMILES: COc1nc(N[C@H]2CCN(C(=O)CO)C[C@H]2F)nn2cc(F)c(-c3cc(F)c4ncn(CCF)c4c3)c12] |
| 240 | 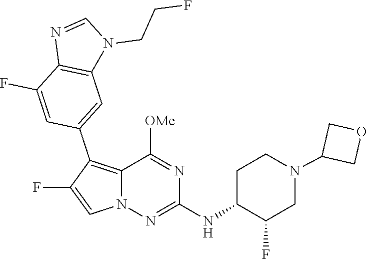 [SMILES: COc1nc(N[C@@H]2CCN(C3COC3)C[C@@H]2F)nn2cc(F)c(-c3cc(F)c4ncn(CCF)c4c3)c12] |
| 241 | 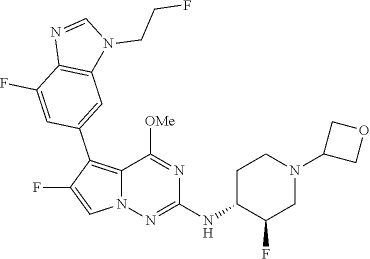 [SMILES: COc1nc(N[C@@H]2CCN(C3COC3)C[C@H]2F)nn2cc(F)c(-c3cc(F)c4ncn(CCF)c4c3)c12] |
| 242 | 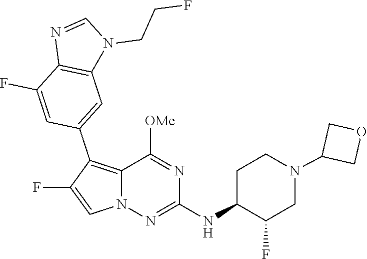 [SMILES: COc1nc(N[C@H]2CCN(C3COC3)C[C@@H]2F)nn2cc(F)c(-c3cc(F)c4ncn(CCF)c4c3)c12] |
| 243 | 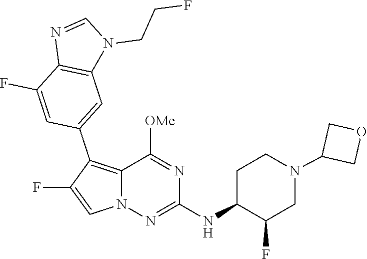 [SMILES: COc1nc(N[C@H]2CCN(C3COC3)C[C@H]2F)nn2cc(F)c(-c3cc(F)c4ncn(CCF)c4c3)c12] |
| 244 | 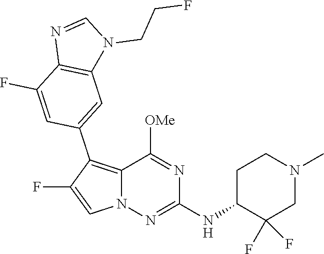 [SMILES: COc1nc(N[C@@H]2CCN(C)CC2(F)F)nn2cc(F)c(-c3cc(F)c4ncn(CCF)c4c3)c12] |
| 245 | 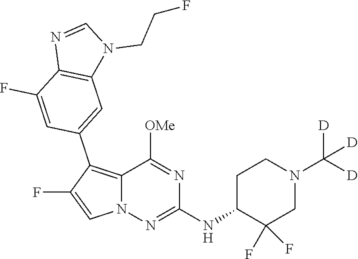 [SMILES: [2H]C([2H])([2H])N1CC[C@@H](Nc2nc(OC)c3c(-c4cc(F)c5ncn(CCF)c5c4)c(F)cn3n2)C(F)(F)C1] |
| 246 | 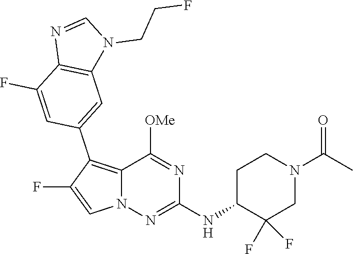 [SMILES: COc1nc(N[C@@H]2CCN(C(C)=O)CC2(F)F)nn2cc(F)c(-c3cc(F)c4ncn(CCF)c4c3)c12] |
| 247 | 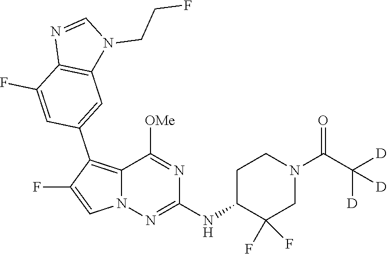 [SMILES: [2H]C([2H])([2H])C(=O)N1CC[C@@H](Nc2nc(OC)c3c(-c4cc(F)c5ncn(CCF)c5c4)c(F)cn3n2)C(F)(F)C1] |
| 248 | 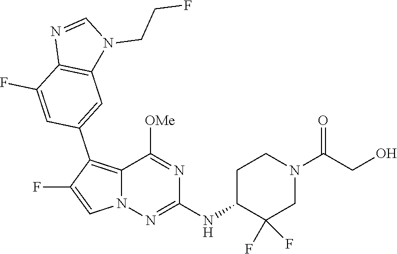 [SMILES: COc1nc(N[C@@H]2CCN(C(=O)CO)CC2(F)F)nn2cc(F)c(-c3cc(F)c4ncn(CCF)c4c3)c12] |
| 249 | 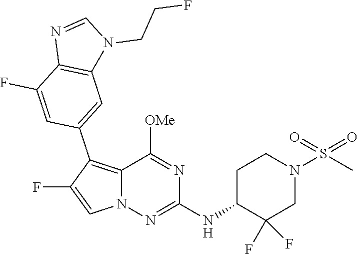 [SMILES: COc1nc(N[C@@H]2CCN(S(C)(=O)=O)CC2(F)F)nn2cc(F)c(-c3cc(F)c4ncn(CCF)c4c3)c12] |
| 250 | 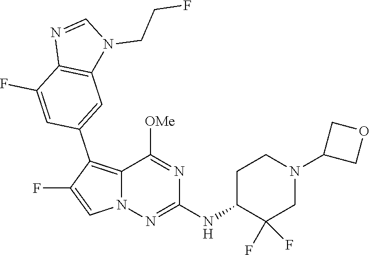 [SMILES: COc1nc(N[C@@H]2CCN(C3COC3)CC2(F)F)nn2cc(F)c(-c3cc(F)c4ncn(CCF)c4c3)c12] |
| 251 | 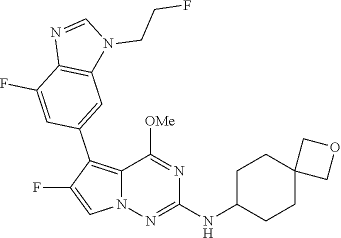 [SMILES: COc1nc(NC2CCC3(CC2)COC3)nn2cc(F)c(-c3cc(F)c4ncn(CCF)c4c3)c12] |
| 252 | 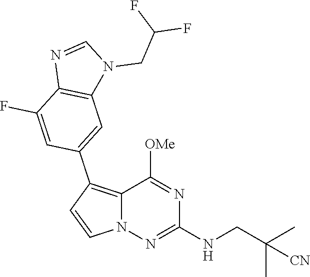 [SMILES: COc1nc(NCC(C)(C)C#N)nn2ccc(-c3cc(F)c4ncn(CC(F)F)c4c3)c12] |
| 253 | 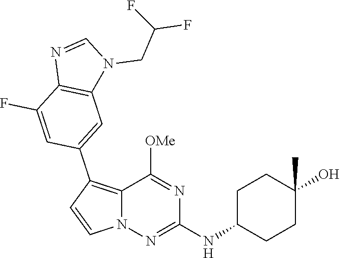 [SMILES: COc1nc(N[C@H]2CC[C@@](C)(O)CC2)nn2ccc(-c3cc(F)c4ncn(CC(F)F)c4c3)c12] |
| 254 | 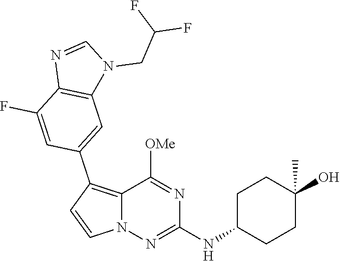 [SMILES: COc1nc(N[C@H]2CC[C@](C)(O)CC2)nn2ccc(-c3cc(F)c4ncn(CC(F)F)c4c3)c12] |
| 255 | 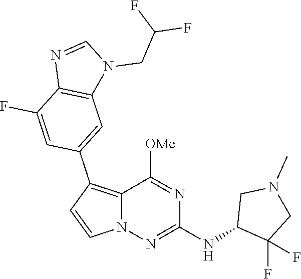 [SMILES: COc1nc(N[C@@H]2CN(C)CC2(F)F)nn2ccc(-c3cc(F)c4ncn(CC(F)F)c4c3)c12] |
| 256 | 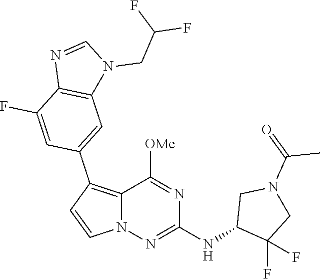 [SMILES: COc1nc(N[C@@H]2CN(C(C)=O)CC2(F)F)nn2ccc(-c3cc(F)c4ncn(CC(F)F)c4c3)c12] |
| 257 | 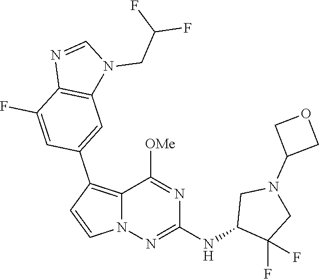 [SMILES: COc1nc(N[C@@H]2CN(C3COC3)CC2(F)F)nn2ccc(-c3cc(F)c4ncn(CC(F)F)c4c3)c12] |
| 258 | 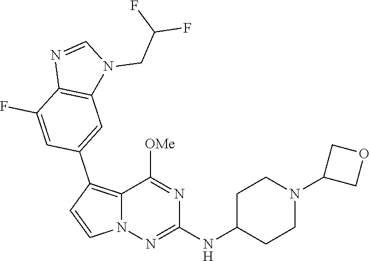 [SMILES: COc1nc(NC2CCN(C3COC3)CC2)nn2ccc(-c3cc(F)c4ncn(CC(F)F)c4c3)c12] |
| 259 | 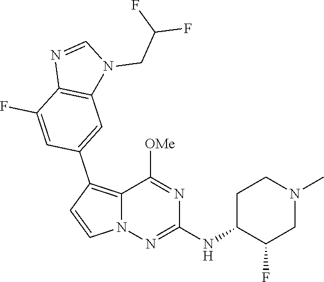 [SMILES: COc1nc(N[C@@H]2CCN(C)C[C@@H]2F)nn2ccc(-c3cc(F)c4ncn(CC(F)F)c4c3)c12] |
| 260 | 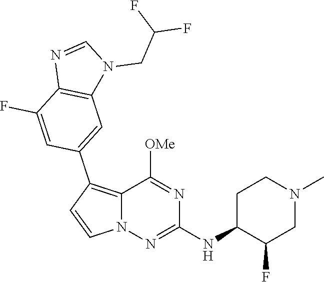 [SMILES: COc1nc(N[C@H]2CCN(C)C[C@H]2F)nn2ccc(-c3cc(F)c4ncn(CC(F)F)c4c3)c12] |
| 261 | 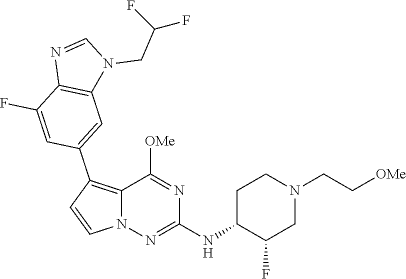 [SMILES: COCCN1CC[C@@H](Nc2nc(OC)c3c(-c4cc(F)c5ncn(CC(F)F)c5c4)ccn3n2)[C@@H](F)C1] |
| 262 | 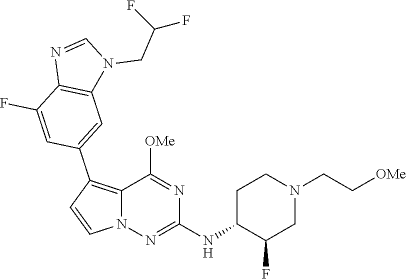 [SMILES: COCCN1CC[C@@H](Nc2nc(OC)c3c(-c4cc(F)c5ncn(CC(F)F)c5c4)ccn3n2)[C@H](F)C1] |
| 263 | 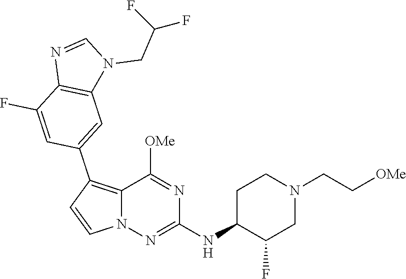 [SMILES: COCCN1CC[C@H](Nc2nc(OC)c3c(-c4cc(F)c5ncn(CC(F)F)c5c4)ccn3n2)[C@@H](F)C1] |
| 264 | 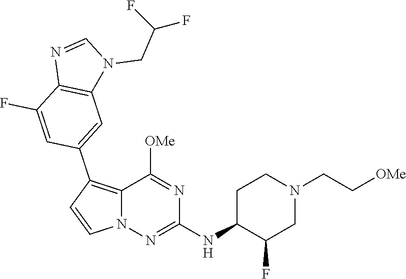 [SMILES: COCCN1CC[C@H](Nc2nc(OC)c3c(-c4cc(F)c5ncn(CC(F)F)c5c4)ccn3n2)[C@H](F)C1] |
| 265 | 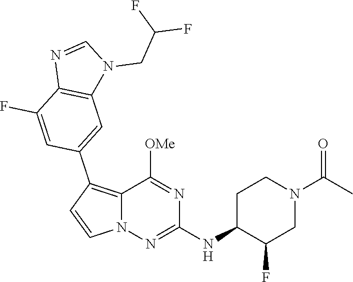 [SMILES: COc1nc(N[C@H]2CCN(C(C)=O)C[C@H]2F)nn2ccc(-c3cc(F)c4ncn(CC(F)F)c4c3)c12] |
| 266 | 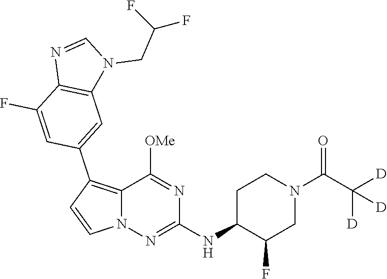 [SMILES: [2H]C([2H])([2H])C(=O)N1CC[C@H](Nc2nc(OC)c3c(-c4cc(F)c5ncn(CC(F)F)c5c4)ccn3n2)[C@H](F)C1] |
| 267 | 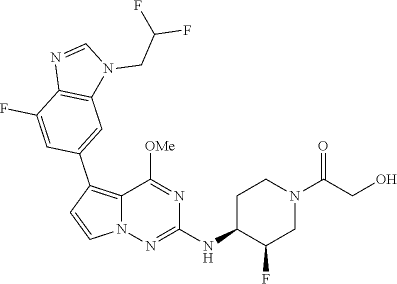 [SMILES: COc1nc(N[C@H]2CCN(C(=O)CO)C[C@H]2F)nn2ccc(-c3cc(F)c4ncn(CC(F)F)c4c3)c12] |
| 268 | 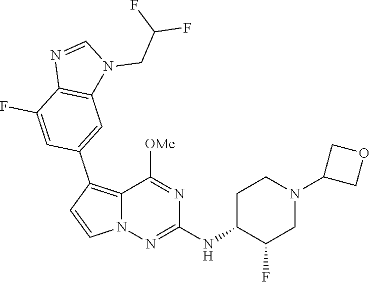 [SMILES: COc1nc(N[C@@H]2CCN(C3COC3)C[C@@H]2F)nn2ccc(-c3cc(F)c4ncn(CC(F)F)c4c3)c12] |
| 269 | 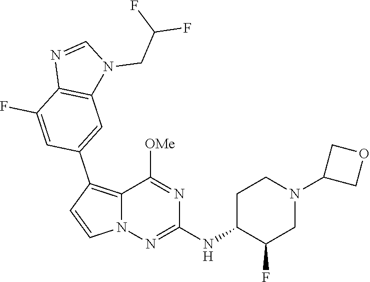 [SMILES: COc1nc(N[C@@H]2CCN(C3COC3)C[C@H]2F)nn2ccc(-c3cc(F)c4ncn(CC(F)F)c4c3)c12] |
| 270 | 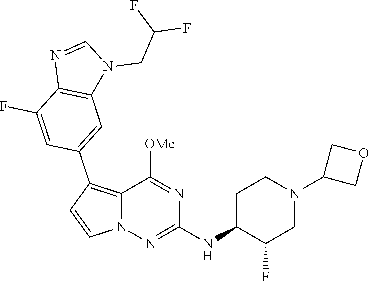 [SMILES: COc1nc(N[C@H]2CCN(C3COC3)C[C@@H]2F)nn2ccc(-c3cc(F)c4ncn(CC(F)F)c4c3)c12] |
| 271 | 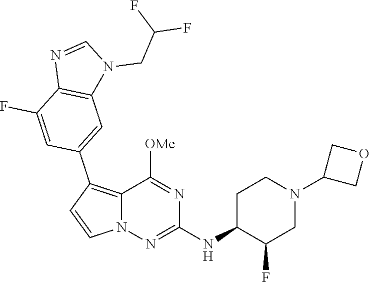 [SMILES: COc1nc(N[C@H]2CCN(C3COC3)C[C@H]2F)nn2ccc(-c3cc(F)c4ncn(CC(F)F)c4c3)c12] |
| 272 | 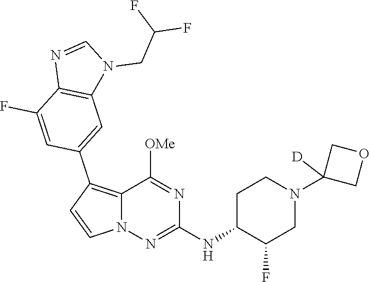 [SMILES: [2H]C1(N2CC[C@@H](Nc3nc(OC)c4c(-c5cc(F)c6ncn(CC(F)F)c6c5)ccn4n3)[C@@H](F)C2)COC1] |
| 273 | 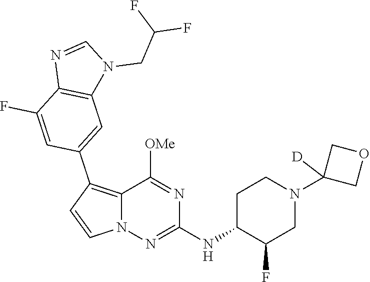 [SMILES: [2H]C1(N2CC[C@@H](Nc3nc(OC)c4c(-c5cc(F)c6ncn(CC(F)F)c6c5)ccn4n3)[C@H](F)C2)COC1] |
| 274 | 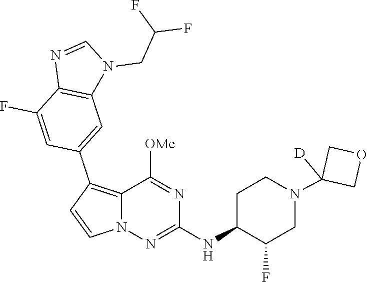 [SMILES: [2H]C1(N2CC[C@H](Nc3nc(OC)c4c(-c5cc(F)c6ncn(CC(F)F)c6c5)ccn4n3)[C@@H](F)C2)COC1] |
| 275 | 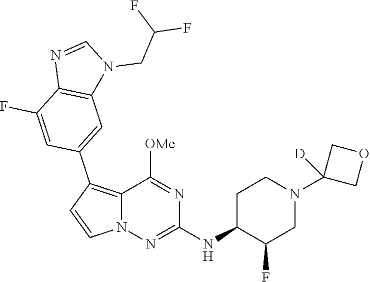 [SMILES: [2H]C1(N2CC[C@H](Nc3nc(OC)c4c(-c5cc(F)c6ncn(CC(F)F)c6c5)ccn4n3)[C@H](F)C2)COC1] |
| 276 | 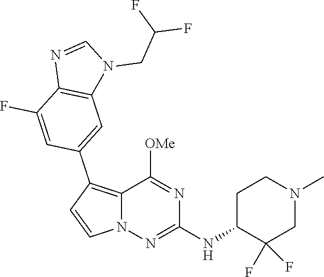 [SMILES: COc1nc(N[C@@H]2CCN(C)CC2(F)F)nn2ccc(-c3cc(F)c4ncn(CC(F)F)c4c3)c12] |
| 277 | 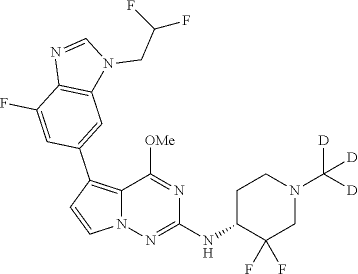 [SMILES: [2H]C([2H])([2H])N1CC[C@@H](Nc2nc(OC)c3c(-c4cc(F)c5ncn(CC(F)F)c5c4)ccn3n2)C(F)(F)C1] |
| 278 | 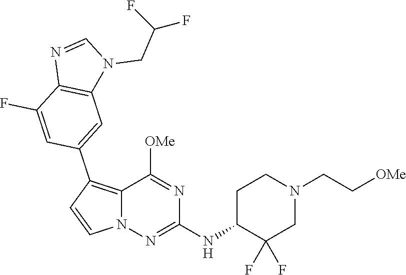 [SMILES: COCCN1CC[C@@H](Nc2nc(OC)c3c(-c4cc(F)c5ncn(CC(F)F)c5c4)ccn3n2)C(F)(F)C1] |
| 279 | 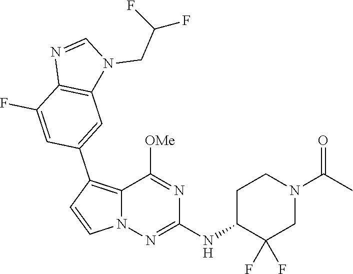 [SMILES: COc1nc(N[C@@H]2CCN(C(C)=O)CC2(F)F)nn2ccc(-c3cc(F)c4ncn(CC(F)F)c4c3)c12] |
| 280 | 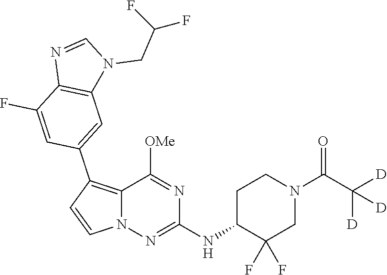 [SMILES: [2H]C([2H])([2H])C(=O)N1CC[C@@H](Nc2nc(OC)c3c(-c4cc(F)c5ncn(CC(F)F)c5c4)ccn3n2)C(F)(F)C1] |
| 281 | 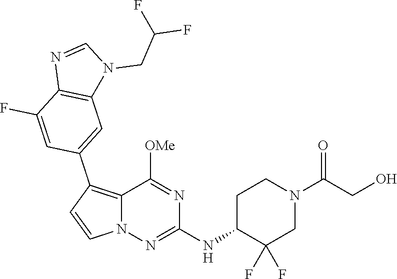 [SMILES: COc1nc(N[C@@H]2CCN(C(=O)CO)CC2(F)F)nn2ccc(-c3cc(F)c4ncn(CC(F)F)c4c3)c12] |
| 282 | 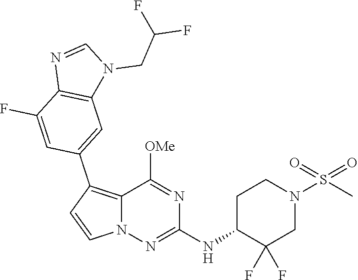 [SMILES: COc1nc(N[C@@H]2CCN(S(C)(=O)=O)CC2(F)F)nn2ccc(-c3cc(F)c4ncn(CC(F)F)c4c3)c12] |
| 283 | 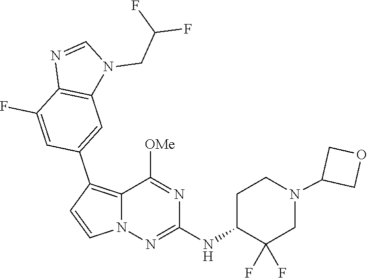 [SMILES: COc1nc(N[C@@H]2CCN(C3COC3)CC2(F)F)nn2ccc(-c3cc(F)c4ncn(CC(F)F)c4c3)c12] |
| 284 | 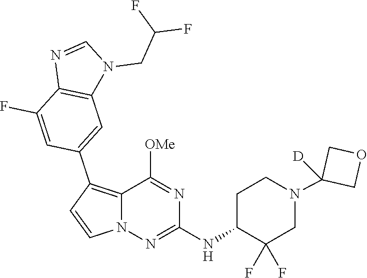 [SMILES: [2H]C1(N2CC[C@@H](Nc3nc(OC)c4c(-c5cc(F)c6ncn(CC(F)F)c6c5)ccn4n3)C(F)(F)C2)COC1] |
| 285 | 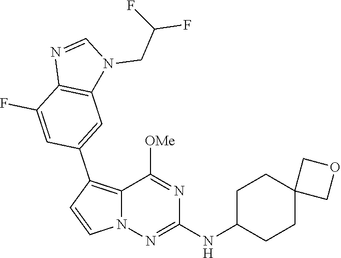 [SMILES: COc1nc(NC2CCC3(CC2)COC3)nn2ccc(-c3cc(F)c4ncn(CC(F)F)c4c3)c12] |
| 286 | 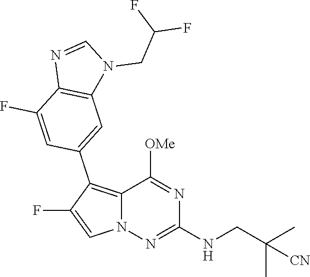 [SMILES: COc1nc(NCC(C)(C)C#N)nn2cc(F)c(-c3cc(F)c4ncn(CC(F)F)c4c3)c12] |
| 287 | 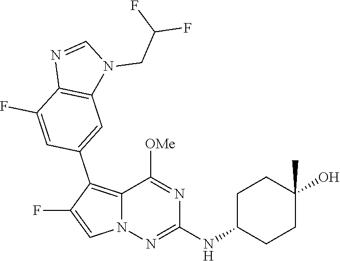 [SMILES: COc1nc(N[C@H]2CC[C@@](C)(O)CC2)nn2cc(F)c(-c3cc(F)c4ncn(CC(F)F)c4c3)c12] |
| 288 | 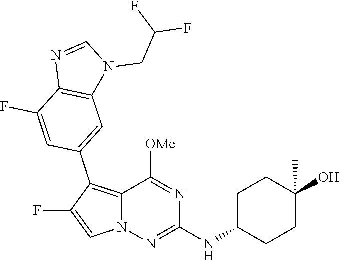 [SMILES: COc1nc(N[C@H]2CC[C@](C)(O)CC2)nn2cc(F)c(-c3cc(F)c4ncn(CC(F)F)c4c3)c12] |
| 289 | 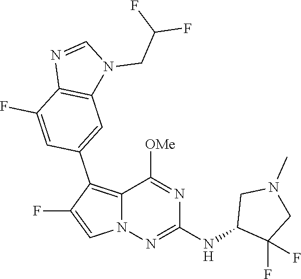 [SMILES: COc1nc(N[C@@H]2CN(C)CC2(F)F)nn2cc(F)c(-c3cc(F)c4ncn(CC(F)F)c4c3)c12] |
| 290 | 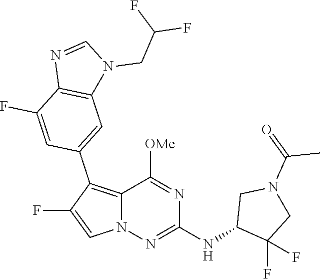 [SMILES: COc1nc(N[C@@H]2CN(C(C)=O)CC2(F)F)nn2cc(F)c(-c3cc(F)c4ncn(CC(F)F)c4c3)c12] |
| 291 | 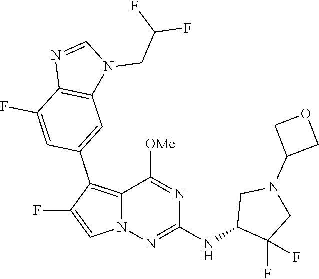 [SMILES: COc1nc(N[C@@H]2CN(C3COC3)CC2(F)F)nn2cc(F)c(-c3cc(F)c4ncn(CC(F)F)c4c3)c12] |
| 292 | 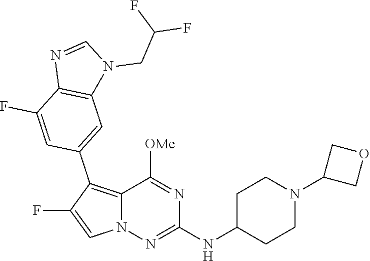 [SMILES: COc1nc(NC2CCN(C3COC3)CC2)nn2cc(F)c(-c3cc(F)c4ncn(CC(F)F)c4c3)c12] |
| 293 | 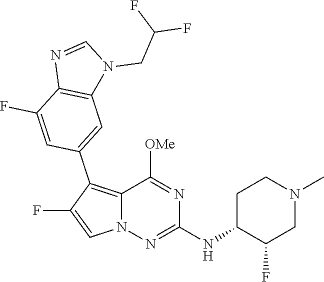 [SMILES: COc1nc(N[C@@H]2CCN(C)C[C@@H]2F)nn2cc(F)c(-c3cc(F)c4ncn(CC(F)F)c4c3)c12] |
| 294 | 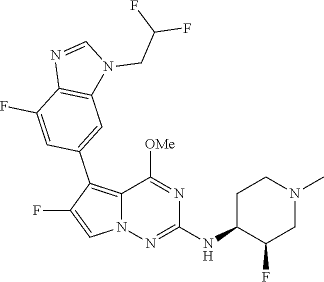 [SMILES: COc1nc(N[C@H]2CCN(C)C[C@H]2F)nn2cc(F)c(-c3cc(F)c4ncn(CC(F)F)c4c3)c12] |
| 295 | 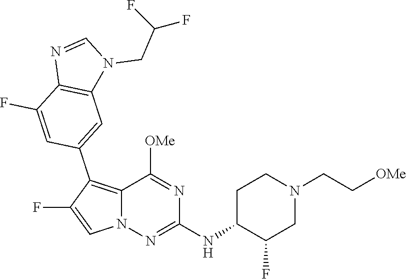 [SMILES: COCCN1CC[C@@H](Nc2nc(OC)c3c(-c4cc(F)c5ncn(CC(F)F)c5c4)c(F)cn3n2)[C@@H](F)C1] |
| 296 | 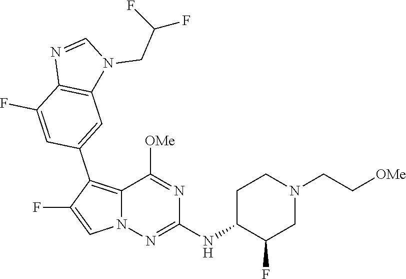 [SMILES: COCCN1CC[C@@H](Nc2nc(OC)c3c(-c4cc(F)c5ncn(CC(F)F)c5c4)c(F)cn3n2)[C@H](F)C1] |
| 297 | 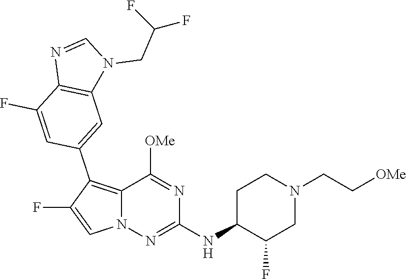 [SMILES: COCCN1CC[C@H](Nc2nc(OC)c3c(-c4cc(F)c5ncn(CC(F)F)c5c4)c(F)cn3n2)[C@@H](F)C1] |
| 298 | 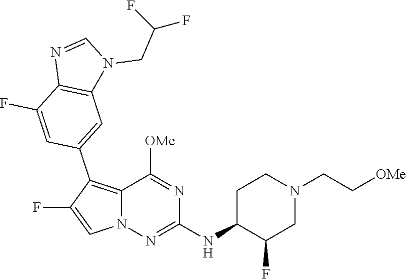 [SMILES: COCCN1CC[C@H](Nc2nc(OC)c3c(-c4cc(F)c5ncn(CC(F)F)c5c4)c(F)cn3n2)[C@H](F)C1] |
| 299 | 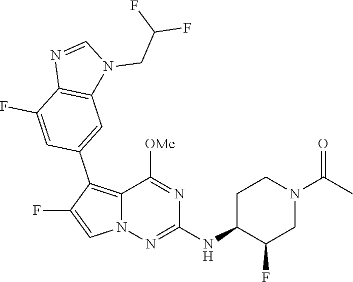 [SMILES: COc1nc(N[C@H]2CCN(C(C)=O)C[C@H]2F)nn2cc(F)c(-c3cc(F)c4ncn(CC(F)F)c4c3)c12] |
| 300 | 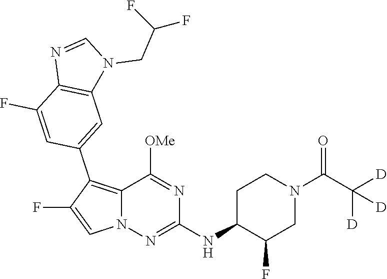 [SMILES: [2H]C([2H])([2H])C(=O)N1CC[C@H](Nc2nc(OC)c3c(-c4cc(F)c5ncn(CC(F)F)c5c4)c(F)cn3n2)[C@H](F)C1] |
| 301 | 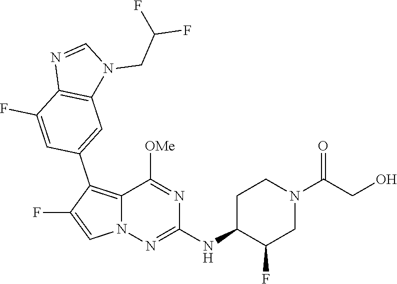 [SMILES: COc1nc(N[C@H]2CCN(C(=O)CO)C[C@H]2F)nn2cc(F)c(-c3cc(F)c4ncn(CC(F)F)c4c3)c12] |
| 302 | 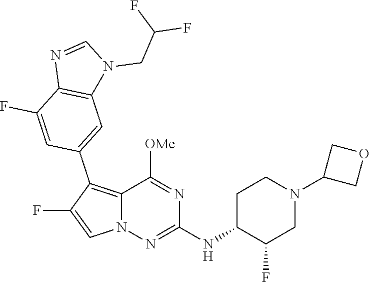 [SMILES: COc1nc(N[C@@H]2CCN(C3COC3)C[C@@H]2F)nn2cc(F)c(-c3cc(F)c4ncn(CC(F)F)c4c3)c12] |
| 303 | 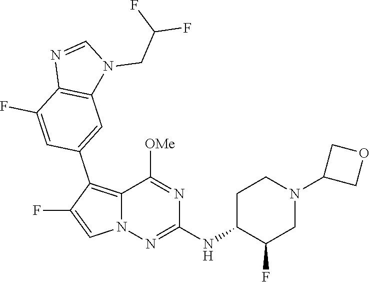 [SMILES: COc1nc(N[C@@H]2CCN(C3COC3)C[C@H]2F)nn2cc(F)c(-c3cc(F)c4ncn(CC(F)F)c4c3)c12] |
| 304 | 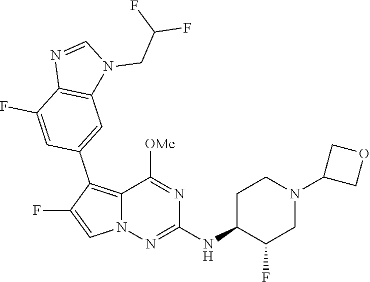 [SMILES: COc1nc(N[C@H]2CCN(C3COC3)C[C@@H]2F)nn2cc(F)c(-c3cc(F)c4ncn(CC(F)F)c4c3)c12] |
| 305 | 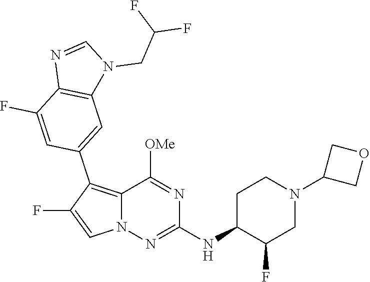 [SMILES: COc1nc(N[C@H]2CCN(C3COC3)C[C@H]2F)nn2cc(F)c(-c3cc(F)c4ncn(CC(F)F)c4c3)c12] |
| 306 | 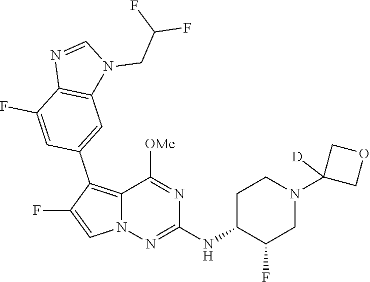 [SMILES: [2H]C1(N2CC[C@@H](Nc3nc(OC)c4c(-c5cc(F)c6ncn(CC(F)F)c6c5)c(F)cn4n3)[C@@H](F)C2)COC1] |
| 307 | 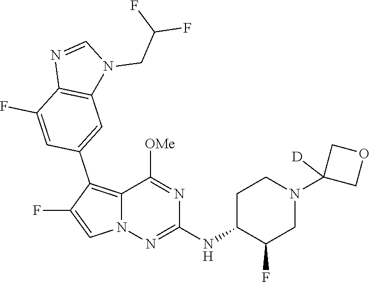 [SMILES: [2H]C1(N2CC[C@@H](Nc3nc(OC)c4c(-c5cc(F)c6ncn(CC(F)F)c6c5)c(F)cn4n3)[C@H](F)C2)COC1] |
| 308 | 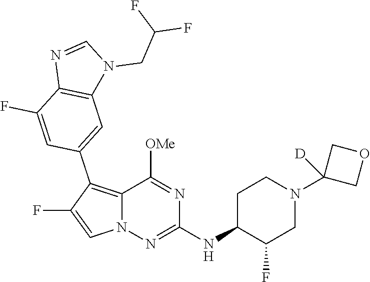 [SMILES: [2H]C1(N2CC[C@H](Nc3nc(OC)c4c(-c5cc(F)c6ncn(CC(F)F)c6c5)c(F)cn4n3)[C@@H](F)C2)COC1] |
| 309 | 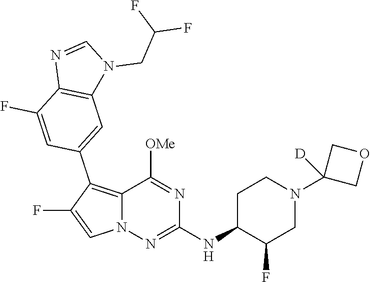 [SMILES: [2H]C1(N2CC[C@H](Nc3nc(OC)c4c(-c5cc(F)c6ncn(CC(F)F)c6c5)c(F)cn4n3)[C@H](F)C2)COC1] |
| 310 | 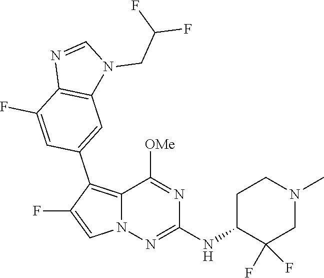 [SMILES: COc1nc(N[C@@H]2CCN(C)CC2(F)F)nn2cc(F)c(-c3cc(F)c4ncn(CC(F)F)c4c3)c12] |
| 311 | 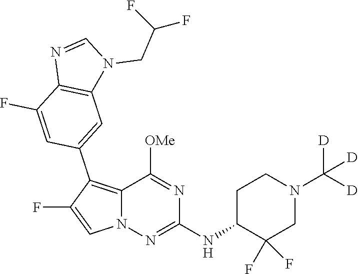 [SMILES: [2H]C([2H])([2H])N1CC[C@@H](Nc2nc(OC)c3c(-c4cc(F)c5ncn(CC(F)F)c5c4)c(F)cn3n2)C(F)(F)C1] |
| 312 | 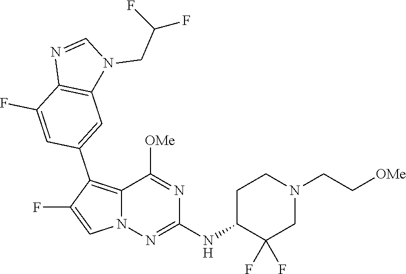 [SMILES: COCCN1CC[C@@H](Nc2nc(OC)c3c(-c4cc(F)c5ncn(CC(F)F)c5c4)c(F)cn3n2)C(F)(F)C1] |
| 313 | 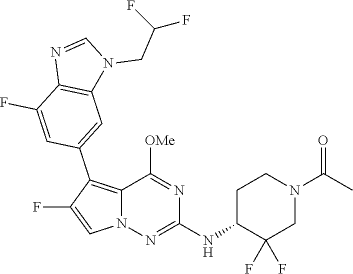 [SMILES: COc1nc(N[C@@H]2CCN(C(C)=O)CC2(F)F)nn2cc(F)c(-c3cc(F)c4ncn(CC(F)F)c4c3)c12] |
| 314 | 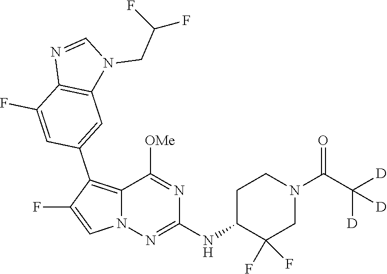 [SMILES: [2H]C([2H])([2H])C(=O)N1CC[C@@H](Nc2nc(OC)c3c(-c4cc(F)c5ncn(CC(F)F)c5c4)c(F)cn3n2)C(F)(F)C1] |
| 315 | 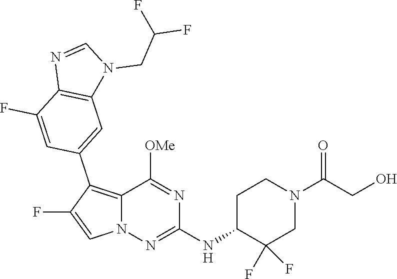 [SMILES: COc1nc(N[C@@H]2CCN(C(=O)CO)CC2(F)F)nn2cc(F)c(-c3cc(F)c4ncn(CC(F)F)c4c3)c12] |
| 316 | 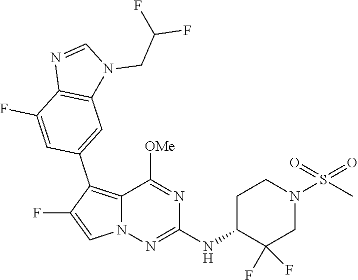 [SMILES: COc1nc(N[C@@H]2CCN(S(C)(=O)=O)CC2(F)F)nn2cc(F)c(-c3cc(F)c4ncn(CC(F)F)c4c3)c12] |
| 317 | 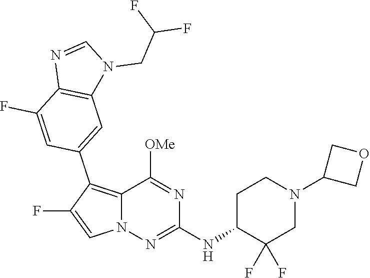 [SMILES: COc1nc(N[C@@H]2CCN(C3COC3)CC2(F)F)nn2cc(F)c(-c3cc(F)c4ncn(CC(F)F)c4c3)c12] |
| 318 | 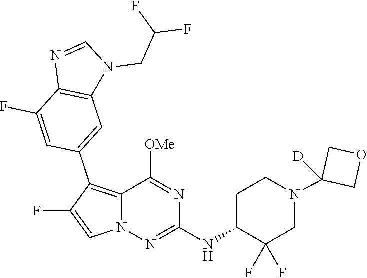 [SMILES: [2H]C1(N2CC[C@@H](Nc3nc(OC)c4c(-c5cc(F)c6ncn(CC(F)F)c6c5)c(F)cn4n3)C(F)(F)C2)COC1] |
| 319 | 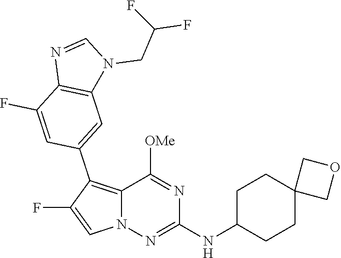 [SMILES: COc1nc(NC2CCC3(CC2)COC3)nn2cc(F)c(-c3cc(F)c4ncn(CC(F)F)c4c3)c12] |
| 320 | 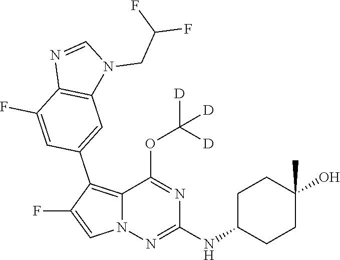 [SMILES: [2H]C([2H])([2H])Oc1nc(N[C@H]2CC[C@@](C)(O)CC2)nn2cc(F)c(-c3cc(F)c4ncn(CC(F)F)c4c3)c12] |
| 321 | 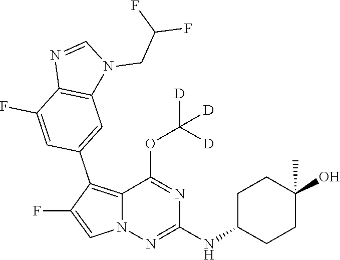 [SMILES: [2H]C([2H])([2H])Oc1nc(N[C@H]2CC[C@](C)(O)CC2)nn2cc(F)c(-c3cc(F)c4ncn(CC(F)F)c4c3)c12] |
| 322 | 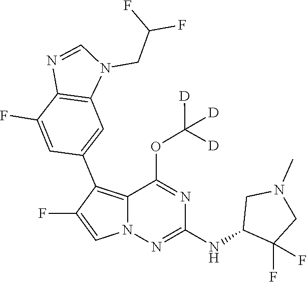 [SMILES: [2H]C([2H])([2H])Oc1nc(N[C@@H]2CN(C)CC2(F)F)nn2cc(F)c(-c3cc(F)c4ncn(CC(F)F)c4c3)c12] |
| 323 | 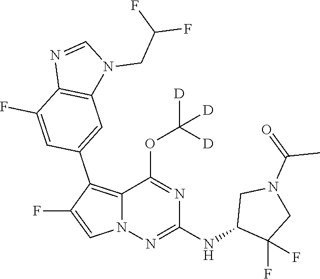 [SMILES: [2H]C([2H])([2H])Oc1nc(N[C@@H]2CN(C(C)=O)CC2(F)F)nn2cc(F)c(-c3cc(F)c4ncn(CC(F)F)c4c3)c12] |
| 324 | 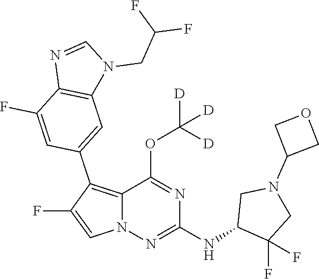 [SMILES: [2H]C([2H])([2H])Oc1nc(N[C@@H]2CN(C3COC3)CC2(F)F)nn2cc(F)c(-c3cc(F)c4ncn(CC(F)F)c4c3)c12] |
| 325 | 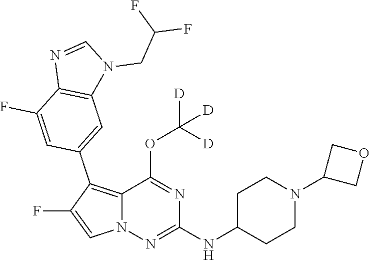 [SMILES: [2H]C([2H])([2H])Oc1nc(NC2CCN(C3COC3)CC2)nn2cc(F)c(-c3cc(F)c4ncn(CC(F)F)c4c3)c12] |
| 326 | 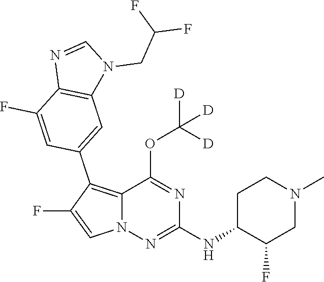 [SMILES: [2H]C([2H])([2H])Oc1nc(N[C@@H]2CCN(C)C[C@@H]2F)nn2cc(F)c(-c3cc(F)c4ncn(CC(F)F)c4c3)c12] |
| 327 | 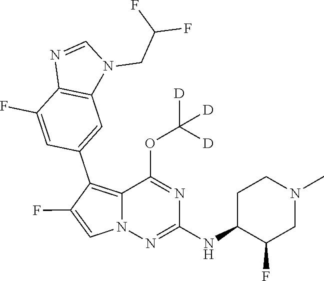 [SMILES: [2H]C([2H])([2H])Oc1nc(N[C@H]2CCN(C)C[C@H]2F)nn2cc(F)c(-c3cc(F)c4ncn(CC(F)F)c4c3)c12] |
| 328 | 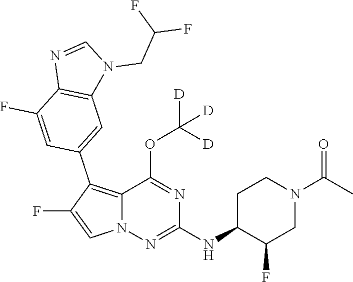 [SMILES: [2H]C([2H])([2H])Oc1nc(N[C@H]2CCN(C(C)=O)C[C@H]2F)nn2cc(F)c(-c3cc(F)c4ncn(CC(F)F)c4c3)c12] |
| 329 | 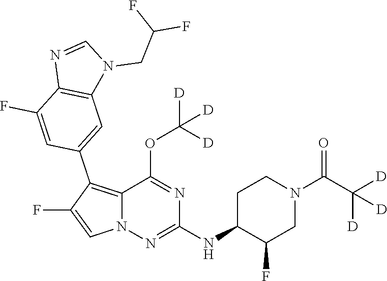 [SMILES: [2H]C([2H])([2H])Oc1nc(N[C@H]2CCN(C(=O)C([2H])([2H])[2H])C[C@H]2F)nn2cc(F)c(-c3cc(F)c4ncn(CC(F)F)c4c3)c12] |
| 330 | 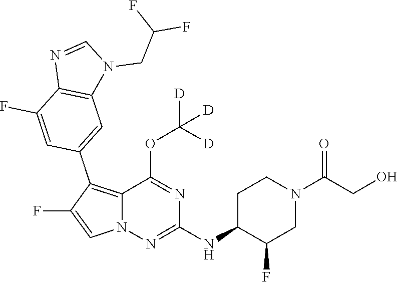 [SMILES: [2H]C([2H])([2H])Oc1nc(N[C@H]2CCN(C(=O)CO)C[C@H]2F)nn2cc(F)c(-c3cc(F)c4ncn(CC(F)F)c4c3)c12] |
| 331 | 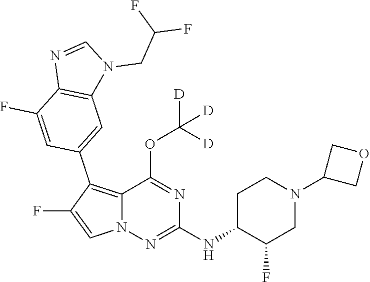 [SMILES: [2H]C([2H])([2H])Oc1nc(N[C@@H]2CCN(C3COC3)C[C@@H]2F)nn2cc(F)c(-c3cc(F)c4ncn(CC(F)F)c4c3)c12] |
| 332 | 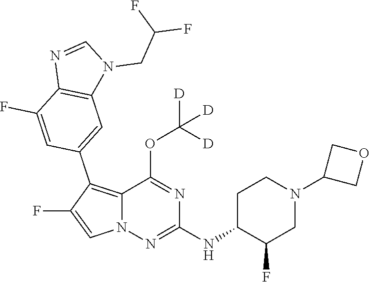 [SMILES: [2H]C([2H])([2H])Oc1nc(N[C@@H]2CCN(C3COC3)C[C@H]2F)nn2cc(F)c(-c3cc(F)c4ncn(CC(F)F)c4c3)c12] |
| 333 | 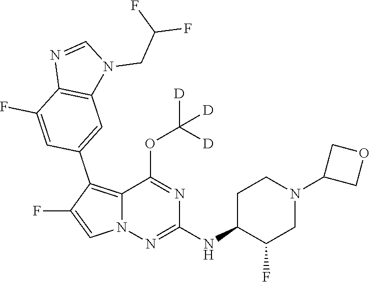 [SMILES: [2H]C([2H])([2H])Oc1nc(N[C@H]2CCN(C3COC3)C[C@@H]2F)nn2cc(F)c(-c3cc(F)c4ncn(CC(F)F)c4c3)c12] |
| 334 | 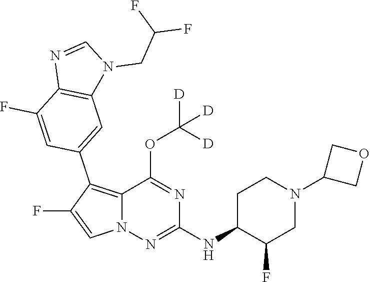 [SMILES: [2H]C([2H])([2H])Oc1nc(N[C@H]2CCN(C3COC3)C[C@H]2F)nn2cc(F)c(-c3cc(F)c4ncn(CC(F)F)c4c3)c12] |
| 335 | 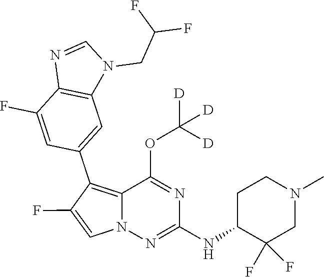 [SMILES: [2H]C([2H])([2H])Oc1nc(N[C@@H]2CCN(C)CC2(F)F)nn2cc(F)c(-c3cc(F)c4ncn(CC(F)F)c4c3)c12] |
| 336 | 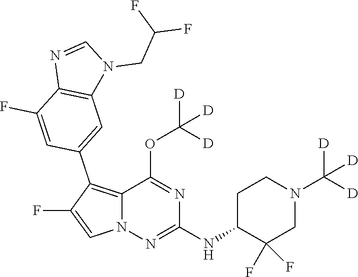 [SMILES: [2H]C([2H])([2H])Oc1nc(N[C@@H]2CCN(C([2H])([2H])[2H])CC2(F)F)nn2cc(F)c(-c3cc(F)c4ncn(CC(F)F)c4c3)c12] |
| 337 | 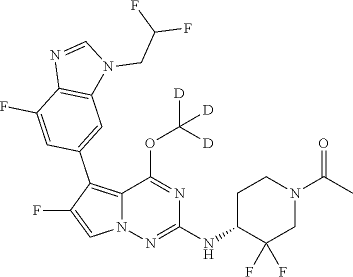 [SMILES: [2H]C([2H])([2H])Oc1nc(N[C@@H]2CCN(C(C)=O)CC2(F)F)nn2cc(F)c(-c3cc(F)c4ncn(CC(F)F)c4c3)c12] |
| 338 | 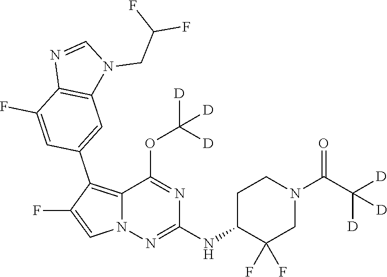 [SMILES: [2H]C([2H])([2H])Oc1nc(N[C@@H]2CCN(C(=O)C([2H])([2H])[2H])CC2(F)F)nn2cc(F)c(-c3cc(F)c4ncn(CC(F)F)c4c3)c12] |
| 339 | 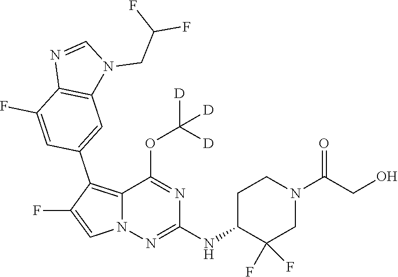 [SMILES: [2H]C([2H])([2H])Oc1nc(N[C@@H]2CCN(C(=O)CO)CC2(F)F)nn2cc(F)c(-c3cc(F)c4ncn(CC(F)F)c4c3)c12] |
| 340 | 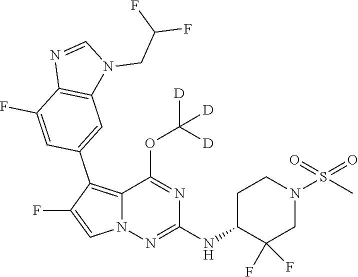 [SMILES: [2H]C([2H])([2H])Oc1nc(N[C@@H]2CCN(S(C)(=O)=O)CC2(F)F)nn2cc(F)c(-c3cc(F)c4ncn(CC(F)F)c4c3)c12] |
| 341 | 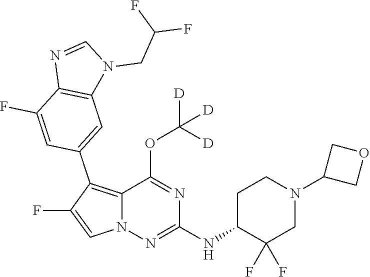 [SMILES: [2H]C([2H])([2H])Oc1nc(N[C@@H]2CCN(C3COC3)CC2(F)F)nn2cc(F)c(-c3cc(F)c4ncn(CC(F)F)c4c3)c12] |
| 342 | 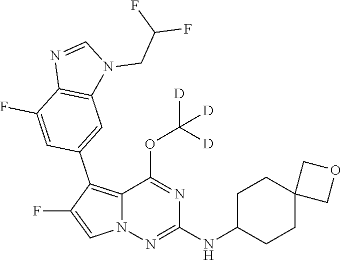 [SMILES: [2H]C([2H])([2H])Oc1nc(NC2CCC3(CC2)COC3)nn2cc(F)c(-c3cc(F)c4ncn(CC(F)F)c4c3)c12] |
| 343 | 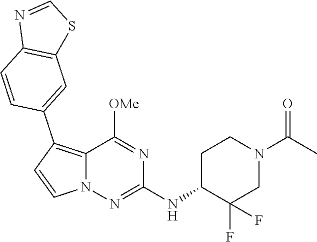 [SMILES: COc1nc(N[C@@H]2CCN(C(C)=O)CC2(F)F)nn2ccc(-c3ccc4ncsc4c3)c12] |
| 344 | 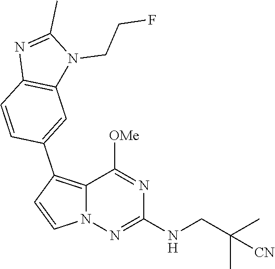 [SMILES: COc1nc(NCC(C)(C)C#N)nn2ccc(-c3ccc4nc(C)n(CCF)c4c3)c12] |
| 345 | 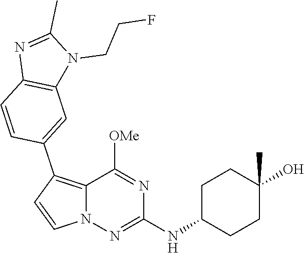 [SMILES: COc1nc(N[C@H]2CC[C@@](C)(O)CC2)nn2ccc(-c3ccc4nc(C)n(CCF)c4c3)c12] |
| 346 | 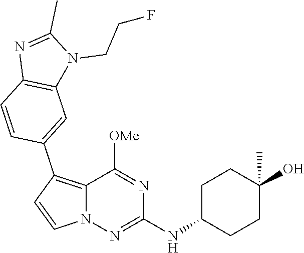 [SMILES: COc1nc(N[C@H]2CC[C@](C)(O)CC2)nn2ccc(-c3ccc4nc(C)n(CCF)c4c3)c12] |
| 347 | 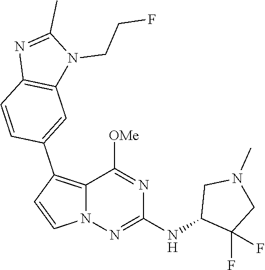 [SMILES: COc1nc(N[C@@H]2CN(C)CC2(F)F)nn2ccc(-c3ccc4nc(C)n(CCF)c4c3)c12] |
| 348 | 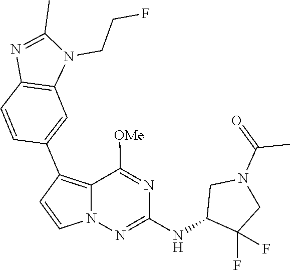 [SMILES: COc1nc(N[C@@H]2CN(C(C)=O)CC2(F)F)nn2ccc(-c3ccc4nc(C)n(CCF)c4c3)c12] |
| 349 | 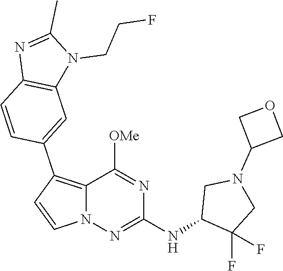 [SMILES: COc1nc(N[C@@H]2CN(C3COC3)CC2(F)F)nn2ccc(-c3ccc4nc(C)n(CCF)c4c3)c12] |
| 350 | 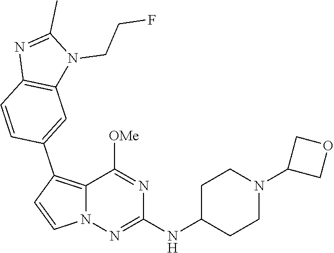 [SMILES: COc1nc(NC2CCN(C3COC3)CC2)nn2ccc(-c3ccc4nc(C)n(CCF)c4c3)c12] |
| 351 | 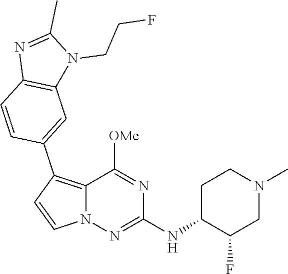 [SMILES: COc1nc(N[C@@H]2CCN(C)C[C@@H]2F)nn2ccc(-c3ccc4nc(C)n(CCF)c4c3)c12] |
| 352 | 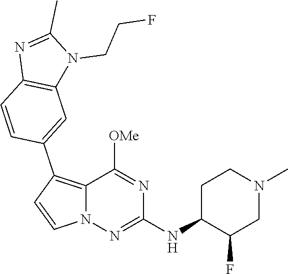 [SMILES: COc1nc(N[C@H]2CCN(C)C[C@H]2F)nn2ccc(-c3ccc4nc(C)n(CCF)c4c3)c12] |
| 353 | 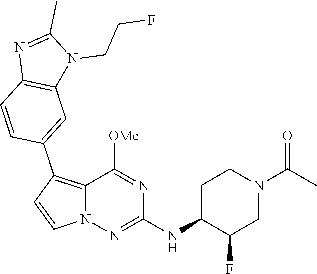 [SMILES: COc1nc(N[C@H]2CCN(C(C)=O)C[C@H]2F)nn2ccc(-c3ccc4nc(C)n(CCF)c4c3)c12] |
| 354 | 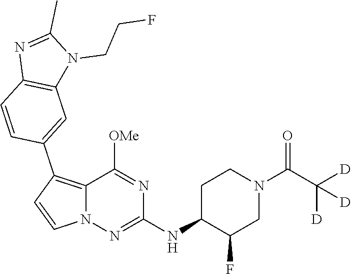 [SMILES: [2H]C([2H])([2H])C(=O)N1CC[C@H](Nc2nc(OC)c3c(-c4ccc5nc(C)n(CCF)c5c4)ccn3n2)[C@H](F)C1] |
| 355 | 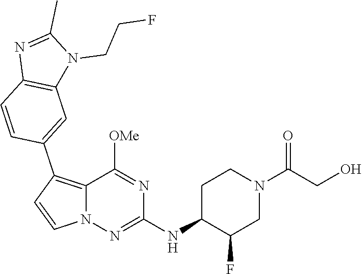 [SMILES: COc1nc(N[C@H]2CCN(C(=O)CO)C[C@H]2F)nn2ccc(-c3ccc4nc(C)n(CCF)c4c3)c12] |
| 356 | 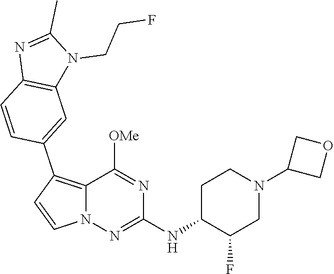 [SMILES: COc1nc(N[C@@H]2CCN(C3COC3)C[C@@H]2F)nn2ccc(-c3ccc4nc(C)n(CCF)c4c3)c12] |
| 357 | 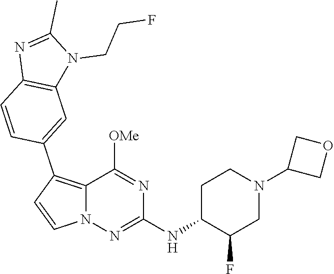 [SMILES: COc1nc(N[C@@H]2CCN(C3COC3)C[C@H]2F)nn2ccc(-c3ccc4nc(C)n(CCF)c4c3)c12] |
| 358 | 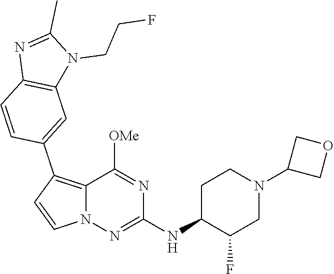 [SMILES: COc1nc(N[C@H]2CCN(C3COC3)C[C@@H]2F)nn2ccc(-c3ccc4nc(C)n(CCF)c4c3)c12] |
| 359 | 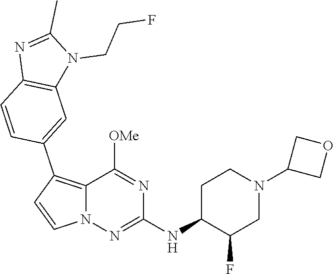 [SMILES: COc1nc(N[C@H]2CCN(C3COC3)C[C@H]2F)nn2ccc(-c3ccc4nc(C)n(CCF)c4c3)c12] |
| 360 | 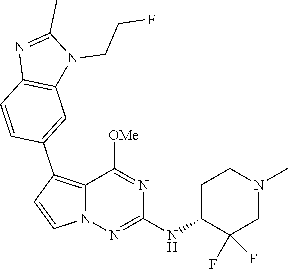 [SMILES: COc1nc(N[C@@H]2CCN(C)CC2(F)F)nn2ccc(-c3ccc4nc(C)n(CCF)c4c3)c12] |
| 361 | 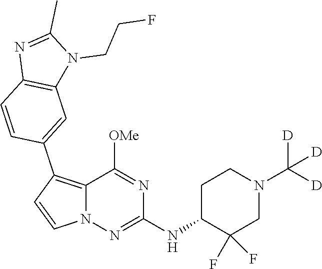 [SMILES: [2H]C([2H])([2H])N1CC[C@@H](Nc2nc(OC)c3c(-c4ccc5nc(C)n(CCF)c5c4)ccn3n2)C(F)(F)C1] |
| 362 | 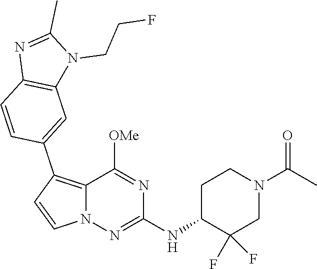 [SMILES: COc1nc(N[C@@H]2CCN(C(C)=O)CC2(F)F)nn2ccc(-c3ccc4nc(C)n(CCF)c4c3)c12] |
| 363 | 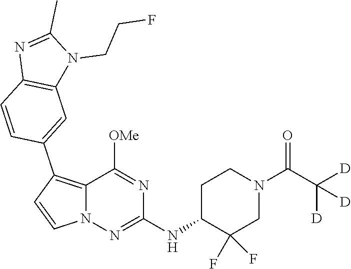 [SMILES: [2H]C([2H])([2H])C(=O)N1CC[C@@H](Nc2nc(OC)c3c(-c4ccc5nc(C)n(CCF)c5c4)ccn3n2)C(F)(F)C1] |
| 364 | 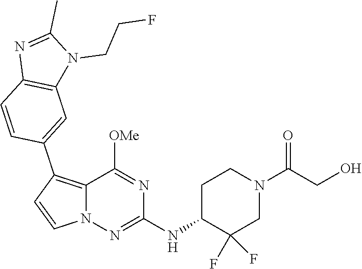 [SMILES: COc1nc(N[C@@H]2CCN(C(=O)CO)CC2(F)F)nn2ccc(-c3ccc4nc(C)n(CCF)c4c3)c12] |
| 365 | 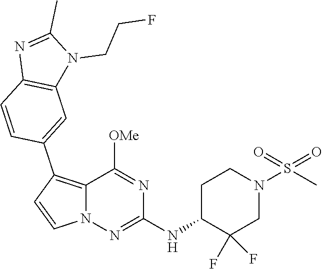 [SMILES: COc1nc(N[C@@H]2CCN(S(C)(=O)=O)CC2(F)F)nn2ccc(-c3ccc4nc(C)n(CCF)c4c3)c12] |
| 366 | 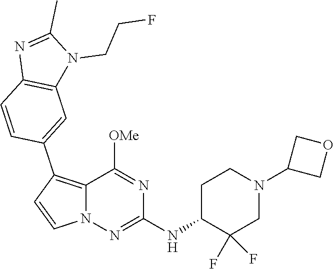 [SMILES: COc1nc(N[C@@H]2CCN(C3COC3)CC2(F)F)nn2ccc(-c3ccc4nc(C)n(CCF)c4c3)c12] |
| 367 | 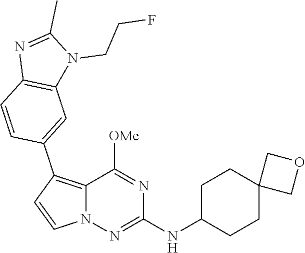 [SMILES: COc1nc(NC2CCC3(CC2)COC3)nn2ccc(-c3ccc4nc(C)n(CCF)c4c3)c12] |
| 368 | 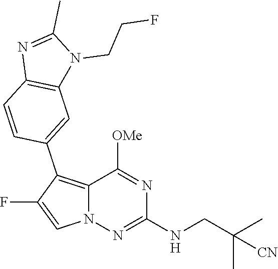 [SMILES: COc1nc(NCC(C)(C)C#N)nn2cc(F)c(-c3ccc4nc(C)n(CCF)c4c3)c12] |
| 369 | 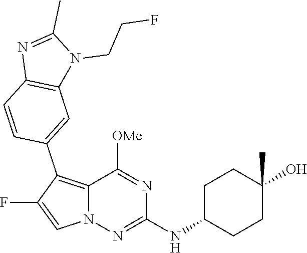 [SMILES: COc1nc(N[C@H]2CC[C@@](C)(O)CC2)nn2cc(F)c(-c3ccc4nc(C)n(CCF)c4c3)c12] |
| 370 | 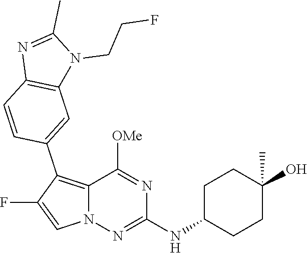 [SMILES: COc1nc(N[C@H]2CC[C@](C)(O)CC2)nn2cc(F)c(-c3ccc4nc(C)n(CCF)c4c3)c12] |
| 371 | 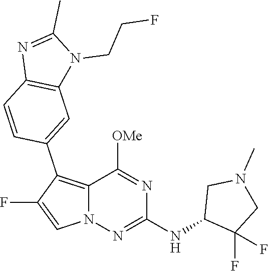 [SMILES: COc1nc(N[C@@H]2CN(C)CC2(F)F)nn2cc(F)c(-c3ccc4nc(C)n(CCF)c4c3)c12] |
| 372 | 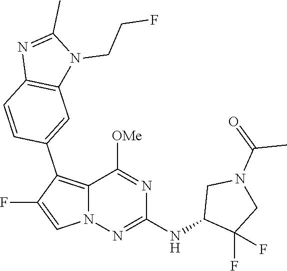 [SMILES: COc1nc(N[C@@H]2CN(C(C)=O)CC2(F)F)nn2cc(F)c(-c3ccc4nc(C)n(CCF)c4c3)c12] |
| 373 | 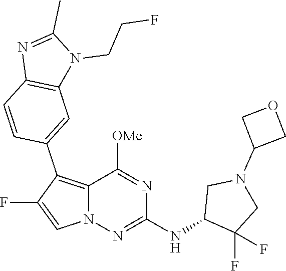 [SMILES: COc1nc(N[C@@H]2CN(C3COC3)CC2(F)F)nn2cc(F)c(-c3ccc4nc(C)n(CCF)c4c3)c12] |
| 374 | 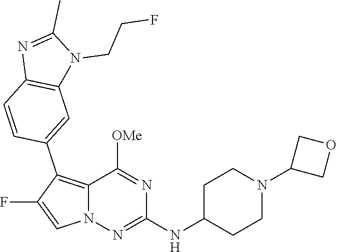 [SMILES: COc1nc(NC2CCN(C3COC3)CC2)nn2cc(F)c(-c3ccc4nc(C)n(CCF)c4c3)c12] |
| 375 | 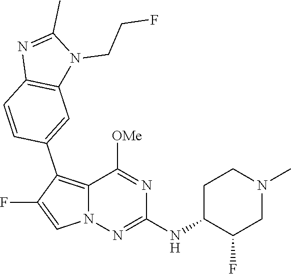 [SMILES: COc1nc(N[C@@H]2CCN(C)C[C@@H]2F)nn2cc(F)c(-c3ccc4nc(C)n(CCF)c4c3)c12] |
| 376 | 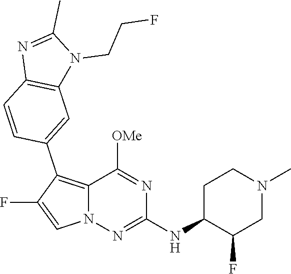 [SMILES: COc1nc(N[C@H]2CCN(C)C[C@H]2F)nn2cc(F)c(-c3ccc4nc(C)n(CCF)c4c3)c12] |
| 377 | 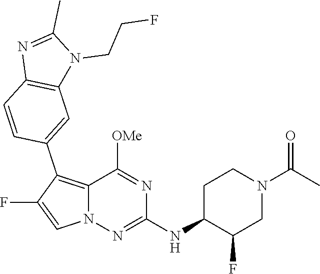 [SMILES: COc1nc(N[C@H]2CCN(C(C)=O)C[C@H]2F)nn2cc(F)c(-c3ccc4nc(C)n(CCF)c4c3)c12] |
| 378 | 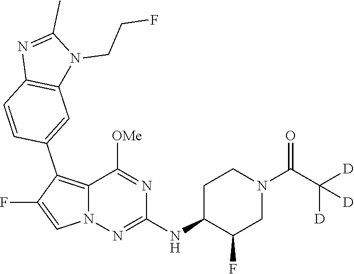 [SMILES: [2H]C([2H])([2H])C(=O)N1CC[C@H](Nc2nc(OC)c3c(-c4ccc5nc(C)n(CCF)c5c4)c(F)cn3n2)[C@H](F)C1] |
| 379 | 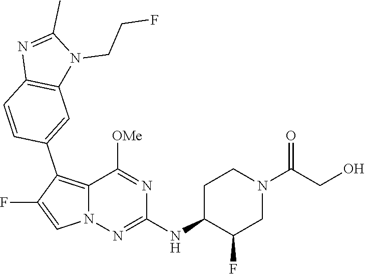 [SMILES: COc1nc(N[C@H]2CCN(C(=O)CO)C[C@H]2F)nn2cc(F)c(-c3ccc4nc(C)n(CCF)c4c3)c12] |
| 380 | 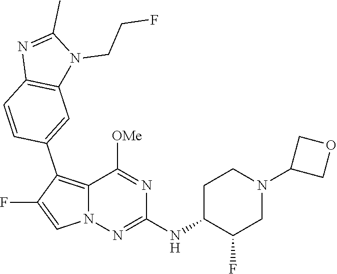 [SMILES: COc1nc(N[C@@H]2CCN(C3COC3)C[C@@H]2F)nn2cc(F)c(-c3ccc4nc(C)n(CCF)c4c3)c12] |
| 381 | 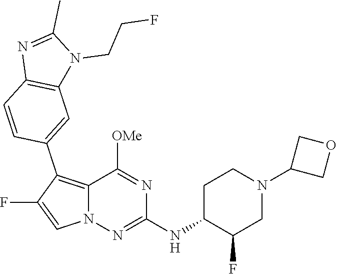 [SMILES: COc1nc(N[C@@H]2CCN(C3COC3)C[C@H]2F)nn2cc(F)c(-c3ccc4nc(C)n(CCF)c4c3)c12] |
| 382 | 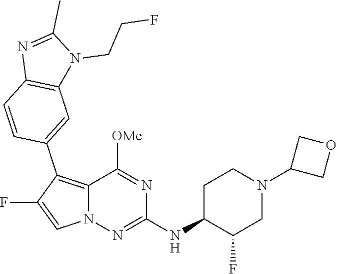 [SMILES: COc1nc(N[C@H]2CCN(C3COC3)C[C@@H]2F)nn2cc(F)c(-c3ccc4nc(C)n(CCF)c4c3)c12] |
| 383 | 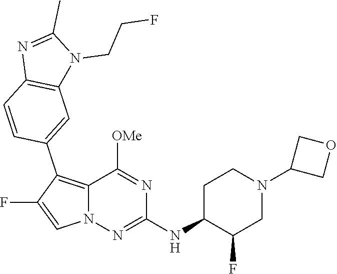 [SMILES: COc1nc(N[C@H]2CCN(C3COC3)C[C@H]2F)nn2cc(F)c(-c3ccc4nc(C)n(CCF)c4c3)c12] |
| 384 | 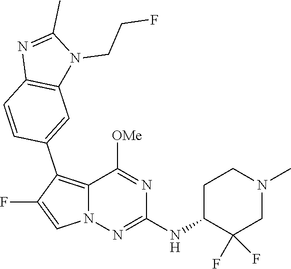 [SMILES: COc1nc(N[C@@H]2CCN(C)CC2(F)F)nn2cc(F)c(-c3ccc4nc(C)n(CCF)c4c3)c12] |
| 385 | 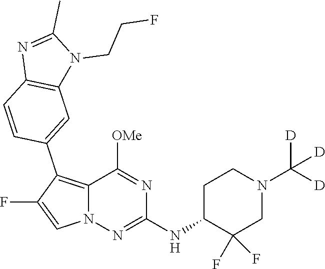 [SMILES: [2H]C([2H])([2H])N1CC[C@@H](Nc2nc(OC)c3c(-c4ccc5nc(C)n(CCF)c5c4)c(F)cn3n2)C(F)(F)C1] |
| 386 | 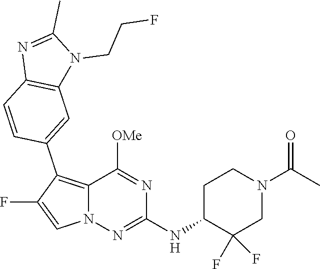 [SMILES: COc1nc(N[C@@H]2CCN(C(C)=O)CC2(F)F)nn2cc(F)c(-c3ccc4nc(C)n(CCF)c4c3)c12] |
| 387 | 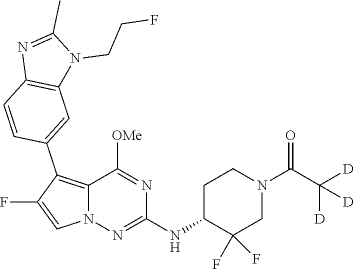 [SMILES: [2H]C([2H])([2H])C(=O)N1CC[C@@H](Nc2nc(OC)c3c(-c4ccc5nc(C)n(CCF)c5c4)c(F)cn3n2)C(F)(F)C1] |
| 388 | 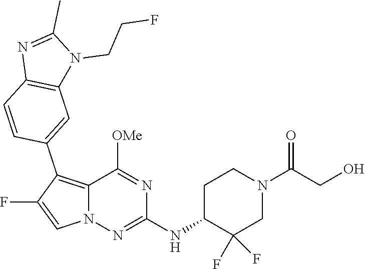 [SMILES: COc1nc(N[C@@H]2CCN(C(=O)CO)CC2(F)F)nn2cc(F)c(-c3ccc4nc(C)n(CCF)c4c3)c12] |
| 389 | 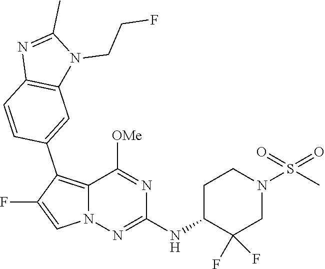 [SMILES: COc1nc(N[C@@H]2CCN(S(C)(=O)=O)CC2(F)F)nn2cc(F)c(-c3ccc4nc(C)n(CCF)c4c3)c12] |
| 390 | 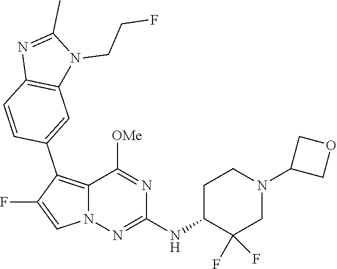 [SMILES: COc1nc(N[C@@H]2CCN(C3COC3)CC2(F)F)nn2cc(F)c(-c3ccc4nc(C)n(CCF)c4c3)c12] |
| 391 | 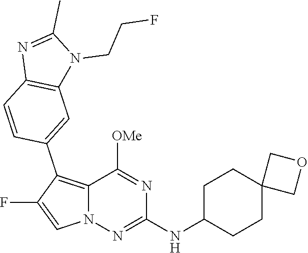 [SMILES: COc1nc(NC2CCC3(CC2)COC3)nn2cc(F)c(-c3ccc4nc(C)n(CCF)c4c3)c12] |
| 392 | 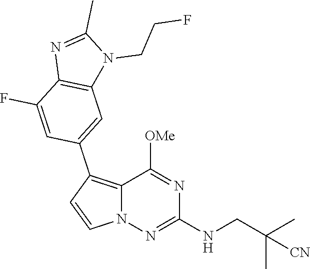 [SMILES: COc1nc(NCC(C)(C)C#N)nn2ccc(-c3cc(F)c4nc(C)n(CCF)c4c3)c12] |
| 393 | 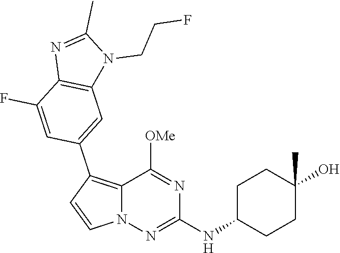 [SMILES: COc1nc(N[C@H]2CC[C@@](C)(O)CC2)nn2ccc(-c3cc(F)c4nc(C)n(CCF)c4c3)c12] |
| 394 | 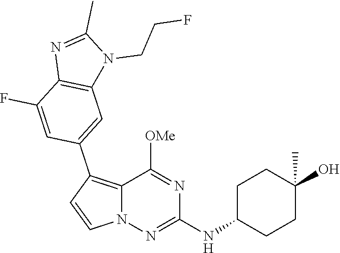 [SMILES: COc1nc(N[C@H]2CC[C@](C)(O)CC2)nn2ccc(-c3cc(F)c4nc(C)n(CCF)c4c3)c12] |
| 395 | 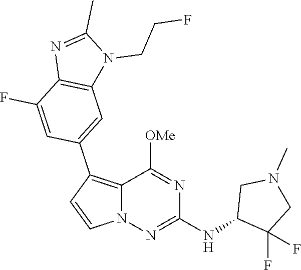 [SMILES: COc1nc(N[C@@H]2CN(C)CC2(F)F)nn2ccc(-c3cc(F)c4nc(C)n(CCF)c4c3)c12] |
| 396 | 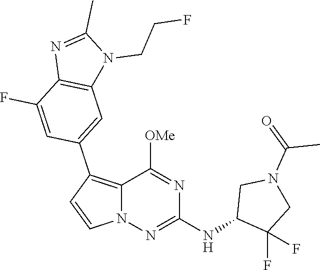 [SMILES: COc1nc(N[C@@H]2CN(C(C)=O)CC2(F)F)nn2ccc(-c3cc(F)c4nc(C)n(CCF)c4c3)c12] |
| 397 | 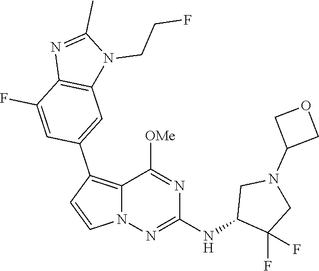 [SMILES: COc1nc(N[C@@H]2CN(C3COC3)CC2(F)F)nn2ccc(-c3cc(F)c4nc(C)n(CCF)c4c3)c12] |
| 398 | 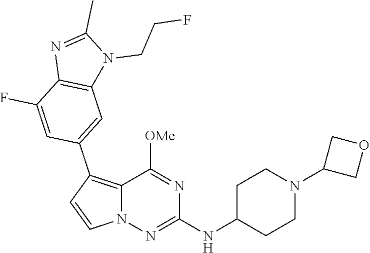 [SMILES: COc1nc(NC2CCN(C3COC3)CC2)nn2ccc(-c3cc(F)c4nc(C)n(CCF)c4c3)c12] |
| 399 | 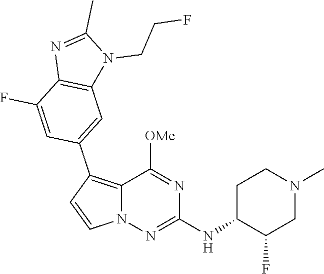 [SMILES: COc1nc(N[C@@H]2CCN(C)C[C@@H]2F)nn2ccc(-c3cc(F)c4nc(C)n(CCF)c4c3)c12] |
| 400 | 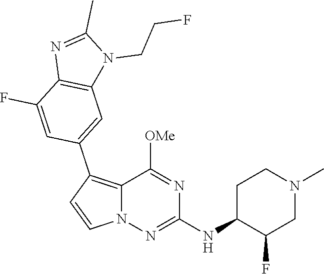 [SMILES: COc1nc(N[C@H]2CCN(C)C[C@H]2F)nn2ccc(-c3cc(F)c4nc(C)n(CCF)c4c3)c12] |
| 401 | 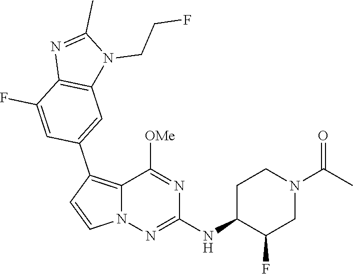 [SMILES: COc1nc(N[C@H]2CCN(C(C)=O)C[C@H]2F)nn2ccc(-c3cc(F)c4nc(C)n(CCF)c4c3)c12] |
| 402 | 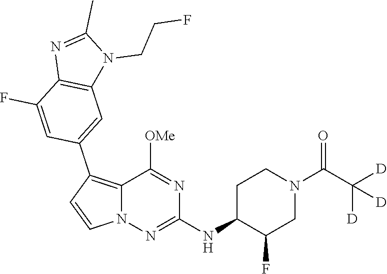 [SMILES: [2H]C([2H])([2H])C(=O)N1CC[C@H](Nc2nc(OC)c3c(-c4cc(F)c5nc(C)n(CCF)c5c4)ccn3n2)[C@H](F)C1] |
| 403 | 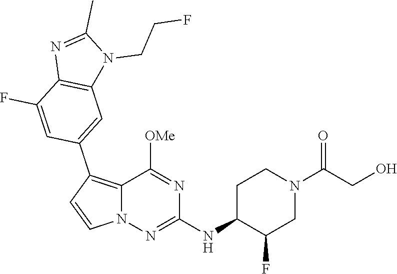 [SMILES: COc1nc(N[C@H]2CCN(C(=O)CO)C[C@H]2F)nn2ccc(-c3cc(F)c4nc(C)n(CCF)c4c3)c12] |
| 404 | 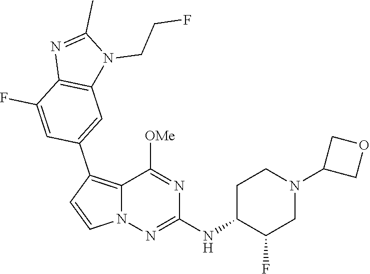 [SMILES: COc1nc(N[C@@H]2CCN(C3COC3)C[C@@H]2F)nn2ccc(-c3cc(F)c4nc(C)n(CCF)c4c3)c12] |
| 405 | 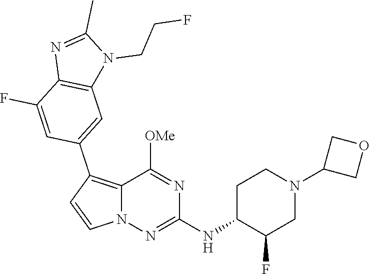 [SMILES: COc1nc(N[C@@H]2CCN(C3COC3)C[C@H]2F)nn2ccc(-c3cc(F)c4nc(C)n(CCF)c4c3)c12] |
| 406 | 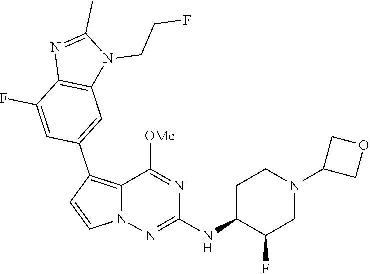 [SMILES: COc1nc(N[C@H]2CCN(C3COC3)C[C@H]2F)nn2ccc(-c3cc(F)c4nc(C)n(CCF)c4c3)c12] |
| 407 | 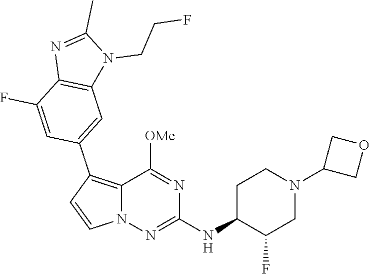 [SMILES: COc1nc(N[C@H]2CCN(C3COC3)C[C@@H]2F)nn2ccc(-c3cc(F)c4nc(C)n(CCF)c4c3)c12] |
| 408 | 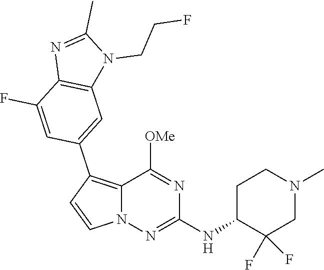 [SMILES: COc1nc(N[C@@H]2CCN(C)CC2(F)F)nn2ccc(-c3cc(F)c4nc(C)n(CCF)c4c3)c12] |
| 409 | 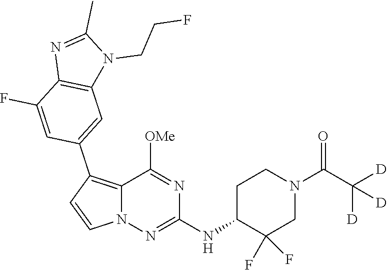 [SMILES: [2H]C([2H])([2H])C(=O)N1CC[C@@H](Nc2nc(OC)c3c(-c4cc(F)c5nc(C)n(CCF)c5c4)ccn3n2)C(F)(F)C1] |
| 410 | 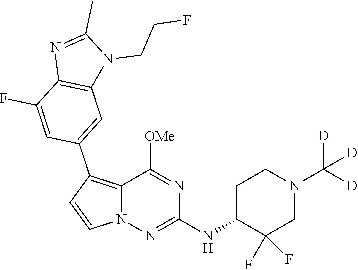 [SMILES: [2H]C([2H])([2H])N1CC[C@@H](Nc2nc(OC)c3c(-c4cc(F)c5nc(C)n(CCF)c5c4)ccn3n2)C(F)(F)C1] |
| 411 | 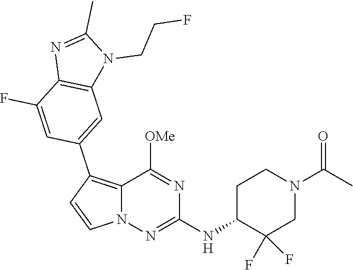 [SMILES: COc1nc(N[C@@H]2CCN(C(C)=O)CC2(F)F)nn2ccc(-c3cc(F)c4nc(C)n(CCF)c4c3)c12] |
| 412 | 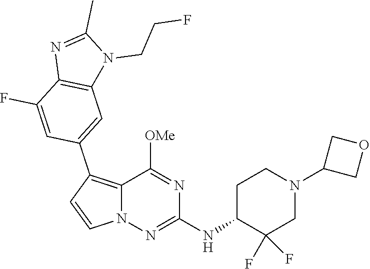 [SMILES: COc1nc(N[C@@H]2CCN(C3COC3)CC2(F)F)nn2ccc(-c3cc(F)c4nc(C)n(CCF)c4c3)c12] |
| 413 | 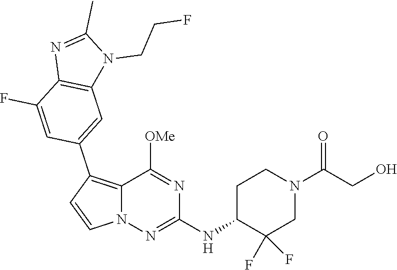 [SMILES: COc1nc(N[C@@H]2CCN(C(=O)CO)CC2(F)F)nn2ccc(-c3cc(F)c4nc(C)n(CCF)c4c3)c12] |
| 414 | 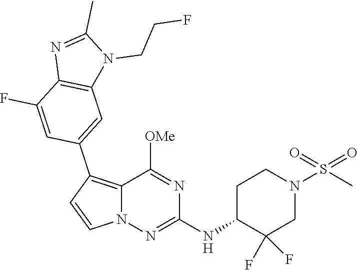 [SMILES: COc1nc(N[C@@H]2CCN(S(C)(=O)=O)CC2(F)F)nn2ccc(-c3cc(F)c4nc(C)n(CCF)c4c3)c12] |
| 415 | 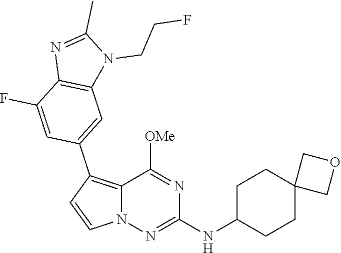 [SMILES: COc1nc(NC2CCC3(CC2)COC3)nn2ccc(-c3cc(F)c4nc(C)n(CCF)c4c3)c12] |
| 416 | 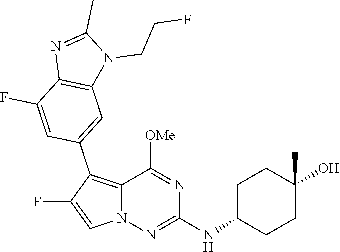 [SMILES: COc1nc(N[C@H]2CC[C@@](C)(O)CC2)nn2cc(F)c(-c3cc(F)c4nc(C)n(CCF)c4c3)c12] |
| 417 | 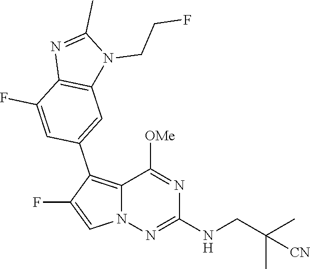 [SMILES: COc1nc(NCC(C)(C)C#N)nn2cc(F)c(-c3cc(F)c4nc(C)n(CCF)c4c3)c12] |
| 418 | 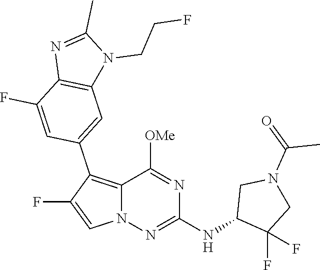 [SMILES: COc1nc(N[C@@H]2CN(C(C)=O)CC2(F)F)nn2cc(F)c(-c3cc(F)c4nc(C)n(CCF)c4c3)c12] |
| 419 | 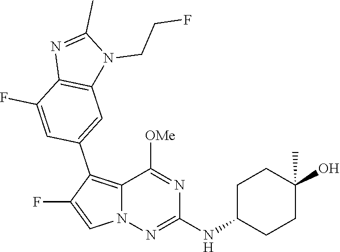 [SMILES: COc1nc(N[C@H]2CC[C@](C)(O)CC2)nn2cc(F)c(-c3cc(F)c4nc(C)n(CCF)c4c3)c12] |
| 420 | 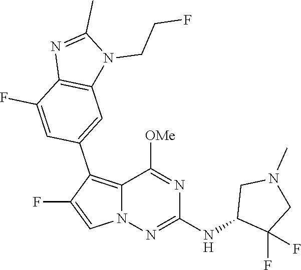 [SMILES: COc1nc(N[C@@H]2CN(C)CC2(F)F)nn2cc(F)c(-c3cc(F)c4nc(C)n(CCF)c4c3)c12] |
| 421 | 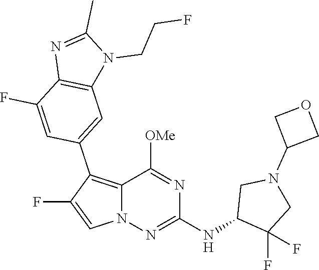 [SMILES: COc1nc(N[C@@H]2CN(C3COC3)CC2(F)F)nn2cc(F)c(-c3cc(F)c4nc(C)n(CCF)c4c3)c12] |
| 422 | 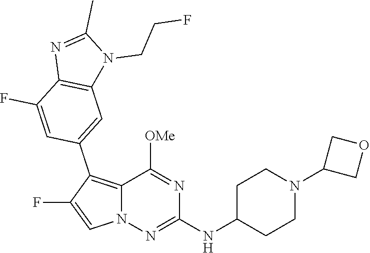 [SMILES: COc1nc(NC2CCN(C3COC3)CC2)nn2cc(F)c(-c3cc(F)c4nc(C)n(CCF)c4c3)c12] |
| 423 | 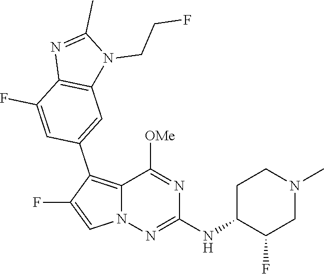 [SMILES: COc1nc(N[C@@H]2CCN(C)C[C@@H]2F)nn2cc(F)c(-c3cc(F)c4nc(C)n(CCF)c4c3)c12] |
| 424 | 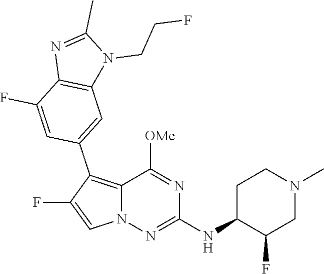 [SMILES: COc1nc(N[C@H]2CCN(C)C[C@H]2F)nn2cc(F)c(-c3cc(F)c4nc(C)n(CCF)c4c3)c12] |
| 425 | 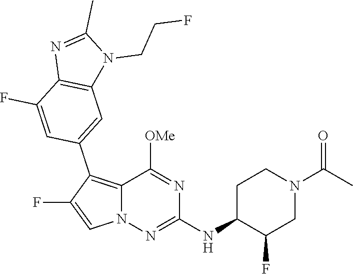 [SMILES: COc1nc(N[C@H]2CCN(C(C)=O)C[C@H]2F)nn2cc(F)c(-c3cc(F)c4nc(C)n(CCF)c4c3)c12] |
| 426 | 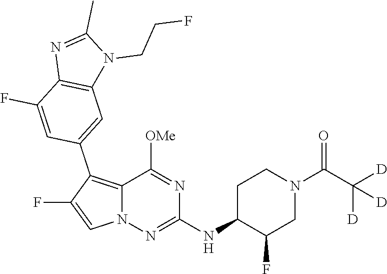 [SMILES: [2H]C([2H])([2H])C(=O)N1CC[C@H](Nc2nc(OC)c3c(-c4cc(F)c5nc(C)n(CCF)c5c4)c(F)cn3n2)[C@H](F)C1] |
| 427 | 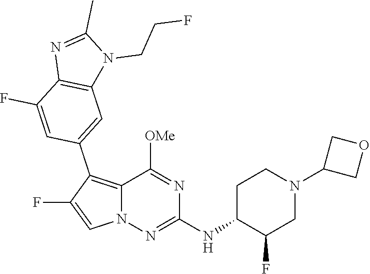 [SMILES: COc1nc(N[C@@H]2CCN(C3COC3)C[C@H]2F)nn2cc(F)c(-c3cc(F)c4nc(C)n(CCF)c4c3)c12] |
| 428 | 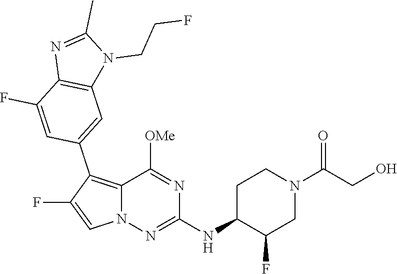 [SMILES: COc1nc(N[C@H]2CCN(C(=O)CO)C[C@H]2F)nn2cc(F)c(-c3cc(F)c4nc(C)n(CCF)c4c3)c12] |
| 429 | 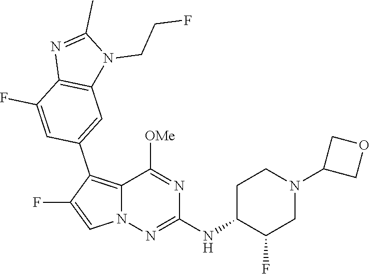 [SMILES: COc1nc(N[C@@H]2CCN(C3COC3)C[C@@H]2F)nn2cc(F)c(-c3cc(F)c4nc(C)n(CCF)c4c3)c12] |
| 430 | 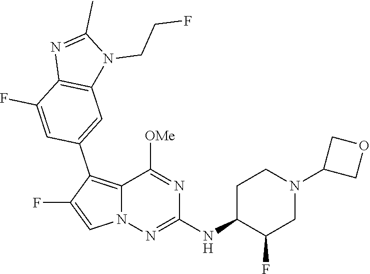 [SMILES: COc1nc(N[C@H]2CCN(C3COC3)C[C@H]2F)nn2cc(F)c(-c3cc(F)c4nc(C)n(CCF)c4c3)c12] |
| 431 | 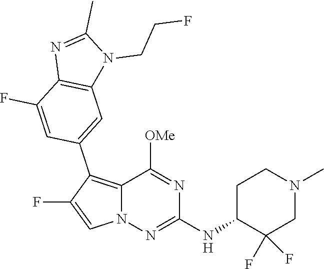 [SMILES: COc1nc(N[C@@H]2CCN(C)CC2(F)F)nn2cc(F)c(-c3cc(F)c4nc(C)n(CCF)c4c3)c12] |
| 432 | 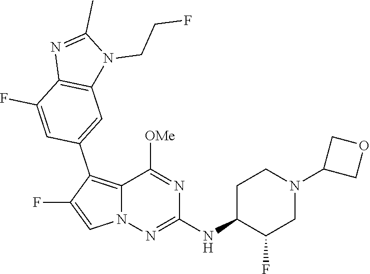 [SMILES: COc1nc(N[C@H]2CCN(C3COC3)C[C@@H]2F)nn2cc(F)c(-c3cc(F)c4nc(C)n(CCF)c4c3)c12] |
| 433 | 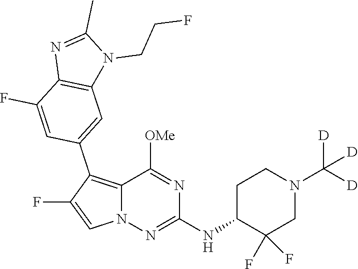 [SMILES: [2H]C([2H])([2H])N1CC[C@@H](Nc2nc(OC)c3c(-c4cc(F)c5nc(C)n(CCF)c5c4)c(F)cn3n2)C(F)(F)C1] |
| 434 | 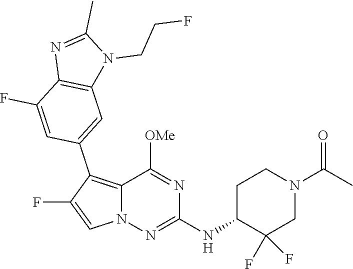 [SMILES: COc1nc(N[C@@H]2CCN(C(C)=O)CC2(F)F)nn2cc(F)c(-c3cc(F)c4nc(C)n(CCF)c4c3)c12] |
| 435 | 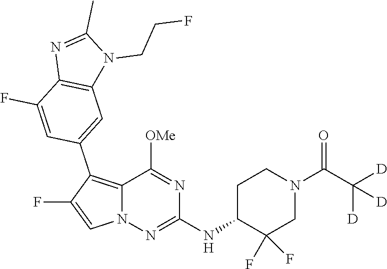 [SMILES: [2H]C([2H])([2H])C(=O)N1CC[C@@H](Nc2nc(OC)c3c(-c4cc(F)c5nc(C)n(CCF)c5c4)c(F)cn3n2)C(F)(F)C1] |
| 436 | 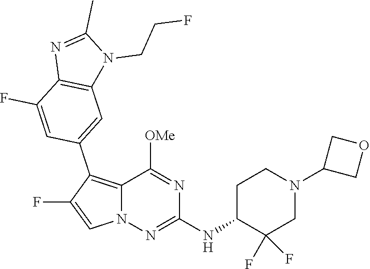 [SMILES: COc1nc(N[C@@H]2CCN(C3COC3)CC2(F)F)nn2cc(F)c(-c3cc(F)c4nc(C)n(CCF)c4c3)c12] |
| 437 | 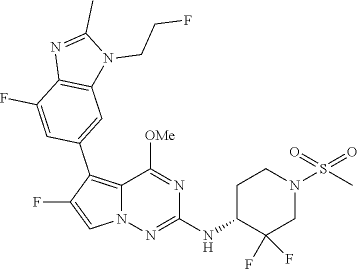 [SMILES: COc1nc(N[C@@H]2CCN(S(C)(=O)=O)CC2(F)F)nn2cc(F)c(-c3cc(F)c4nc(C)n(CCF)c4c3)c12] |
| 438 | 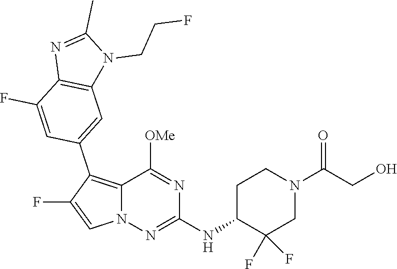 [SMILES: COc1nc(N[C@@H]2CCN(C(=O)CO)CC2(F)F)nn2cc(F)c(-c3cc(F)c4nc(C)n(CCF)c4c3)c12] |
| 439 | 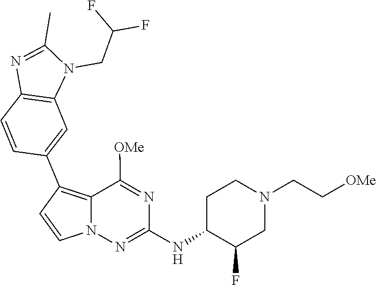 [SMILES: COCCN1CC[C@@H](Nc2nc(OC)c3c(-c4ccc5nc(C)n(CC(F)F)c5c4)ccn3n2)[C@H](F)C1] |
| 440 | 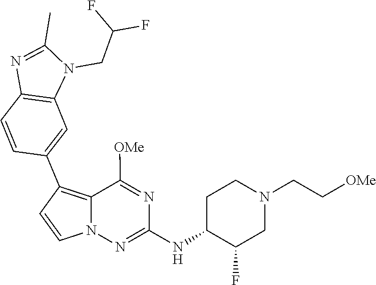 [SMILES: COCCN1CC[C@@H](Nc2nc(OC)c3c(-c4ccc5nc(C)n(CC(F)F)c5c4)ccn3n2)[C@@H](F)C1] |
| 441 | 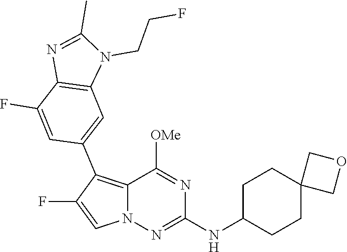 [SMILES: COc1nc(NC2CCC3(CC2)COC3)nn2cc(F)c(-c3cc(F)c4nc(C)n(CCF)c4c3)c12] |
| 442 | 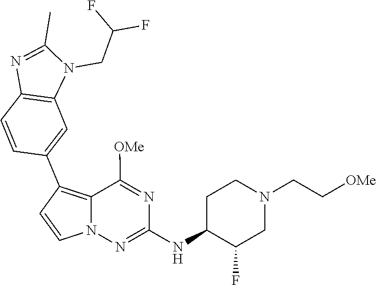 [SMILES: COCCN1CC[C@H](Nc2nc(OC)c3c(-c4ccc5nc(C)n(CC(F)F)c5c4)ccn3n2)[C@@H](F)C1] |
| 443 | 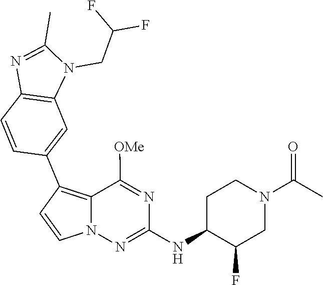 [SMILES: COc1nc(N[C@H]2CCN(C(C)=O)C[C@H]2F)nn2ccc(-c3ccc4nc(C)n(CC(F)F)c4c3)c12] |
| 444 | 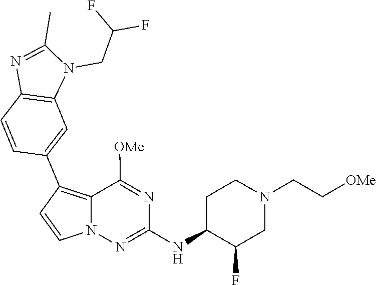 [SMILES: COCCN1CC[C@H](Nc2nc(OC)c3c(-c4ccc5nc(C)n(CC(F)F)c5c4)ccn3n2)[C@H](F)C1] |
| 445 | 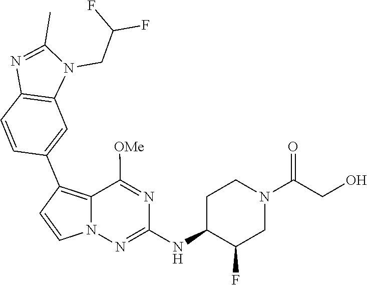 [SMILES: COc1nc(N[C@H]2CCN(C(=O)CO)C[C@H]2F)nn2ccc(-c3ccc4nc(C)n(CC(F)F)c4c3)c12] |
| 446 | 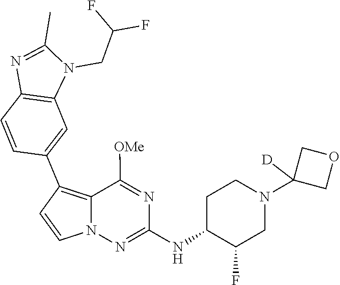 [SMILES: [2H]C1(N2CC[C@@H](Nc3nc(OC)c4c(-c5ccc6nc(C)n(CC(F)F)c6c5)ccn4n3)[C@@H](F)C2)COC1] |
| 447 | 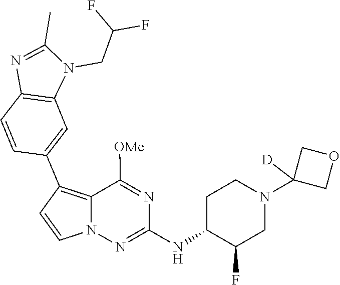 [SMILES: [2H]C1(N2CC[C@@H](Nc3nc(OC)c4c(-c5ccc6nc(C)n(CC(F)F)c6c5)ccn4n3)[C@H](F)C2)COC1] |
| 448 | 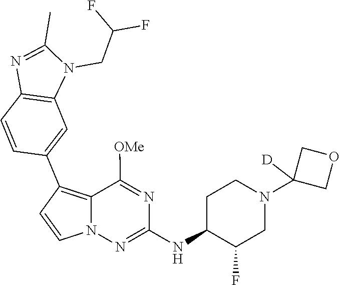 [SMILES: [2H]C1(N2CC[C@H](Nc3nc(OC)c4c(-c5ccc6nc(C)n(CC(F)F)c6c5)ccn4n3)[C@@H](F)C2)COC1] |
| 449 | 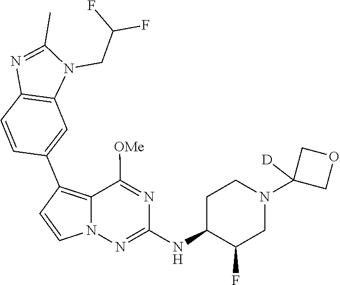 [SMILES: [2H]C1(N2CC[C@H](Nc3nc(OC)c4c(-c5ccc6nc(C)n(CC(F)F)c6c5)ccn4n3)[C@H](F)C2)COC1] |
| 450 | 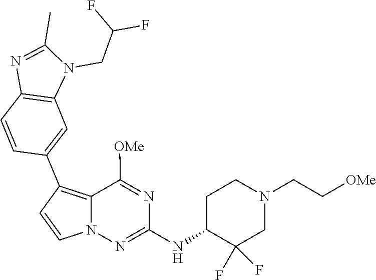 [SMILES: COCCN1CC[C@@H](Nc2nc(OC)c3c(-c4ccc5nc(C)n(CC(F)F)c5c4)ccn3n2)C(F)(F)C1] |
| 451 | 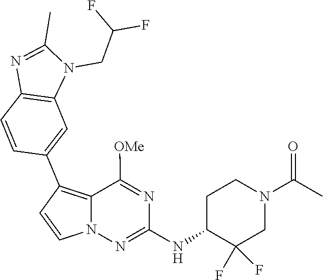 [SMILES: COc1nc(N[C@@H]2CCN(C(C)=O)CC2(F)F)nn2ccc(-c3ccc4nc(C)n(CC(F)F)c4c3)c12] |
| 452 | 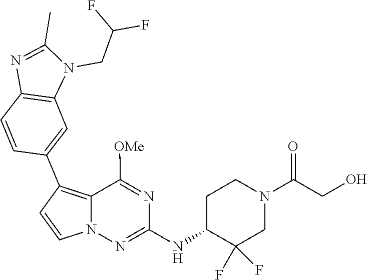 [SMILES: COc1nc(N[C@@H]2CCN(C(=O)CO)CC2(F)F)nn2ccc(-c3ccc4nc(C)n(CC(F)F)c4c3)c12] |
| 453 | 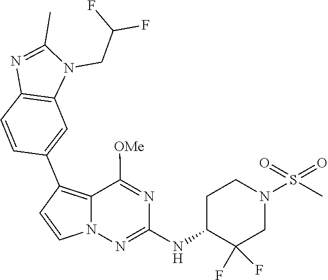 [SMILES: COc1nc(N[C@@H]2CCN(S(C)(=O)=O)CC2(F)F)nn2ccc(-c3ccc4nc(C)n(CC(F)F)c4c3)c12] |
| 454 | 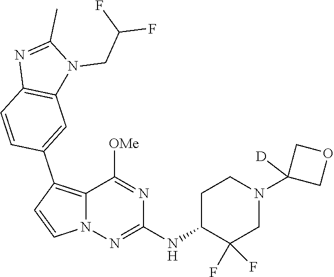 [SMILES: [2H]C1(N2CC[C@@H](Nc3nc(OC)c4c(-c5ccc6nc(C)n(CC(F)F)c6c5)ccn4n3)C(F)(F)C2)COC1] |
| 455 | 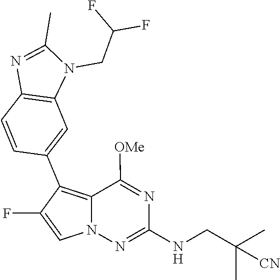 [SMILES: COc1nc(NCC(C)(C)C#N)nn2cc(F)c(-c3ccc4nc(C)n(CC(F)F)c4c3)c12] |
| 456 | 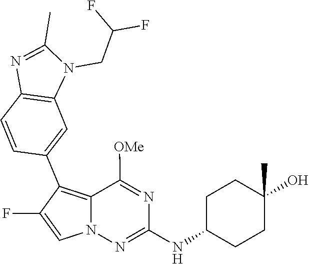 [SMILES: COc1nc(N[C@H]2CC[C@@](C)(O)CC2)nn2cc(F)c(-c3ccc4nc(C)n(CC(F)F)c4c3)c12] |
| 457 | 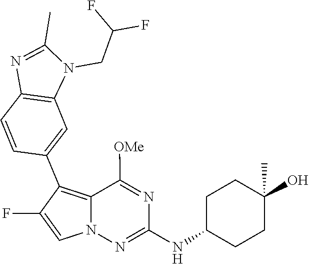 [SMILES: COc1nc(N[C@H]2CC[C@](C)(O)CC2)nn2cc(F)c(-c3ccc4nc(C)n(CC(F)F)c4c3)c12] |
| 458 | 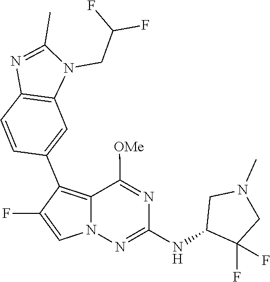 [SMILES: COc1nc(N[C@@H]2CN(C)CC2(F)F)nn2cc(F)c(-c3ccc4nc(C)n(CC(F)F)c4c3)c12] |
| 459 | 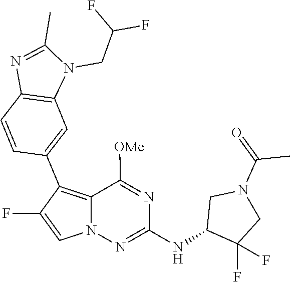 [SMILES: COc1nc(N[C@@H]2CN(C(C)=O)CC2(F)F)nn2cc(F)c(-c3ccc4nc(C)n(CC(F)F)c4c3)c12] |
| 460 | 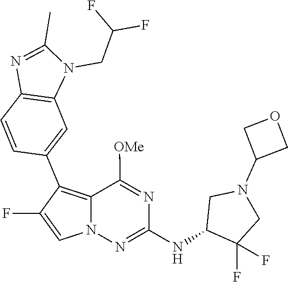 [SMILES: COc1nc(N[C@@H]2CN(C3COC3)CC2(F)F)nn2cc(F)c(-c3ccc4nc(C)n(CC(F)F)c4c3)c12] |
| 461 | 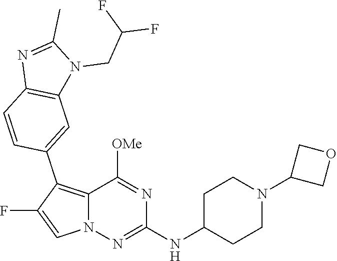 [SMILES: COc1nc(NC2CCN(C3COC3)CC2)nn2cc(F)c(-c3ccc4nc(C)n(CC(F)F)c4c3)c12] |
| 462 | 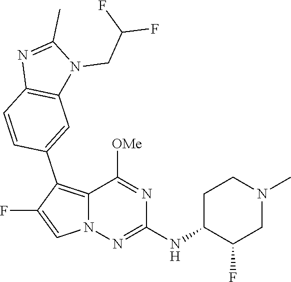 [SMILES: COc1nc(N[C@@H]2CCN(C)C[C@@H]2F)nn2cc(F)c(-c3ccc4nc(C)n(CC(F)F)c4c3)c12] |
| 463 | 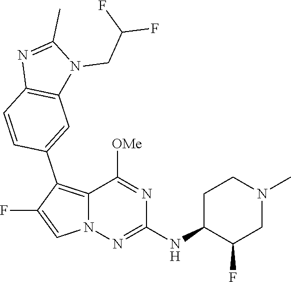 [SMILES: COc1nc(N[C@H]2CCN(C)C[C@H]2F)nn2cc(F)c(-c3ccc4nc(C)n(CC(F)F)c4c3)c12] |
| 464 | 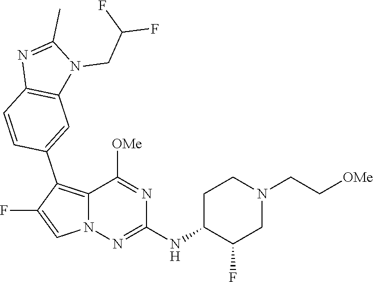 [SMILES: COCCN1CC[C@@H](Nc2nc(OC)c3c(-c4ccc5nc(C)n(CC(F)F)c5c4)c(F)cn3n2)[C@@H](F)C1] |
| 465 | 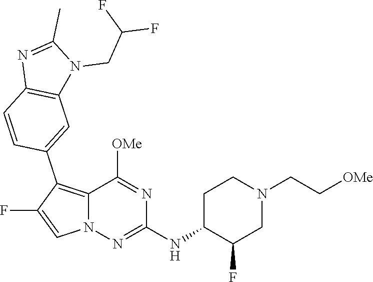 [SMILES: COCCN1CC[C@@H](Nc2nc(OC)c3c(-c4ccc5nc(C)n(CC(F)F)c5c4)c(F)cn3n2)[C@H](F)C1] |
| 466 | 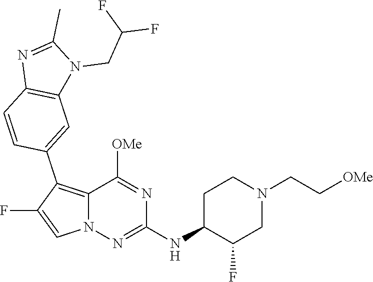 [SMILES: COCCN1CC[C@H](Nc2nc(OC)c3c(-c4ccc5nc(C)n(CC(F)F)c5c4)c(F)cn3n2)[C@@H](F)C1] |
| 467 | 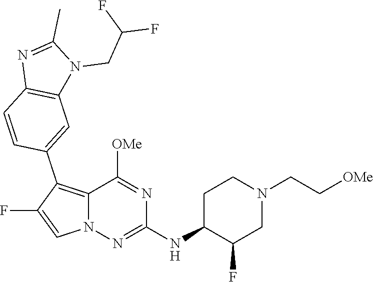 [SMILES: COCCN1CC[C@H](Nc2nc(OC)c3c(-c4ccc5nc(C)n(CC(F)F)c5c4)c(F)cn3n2)[C@H](F)C1] |
| 468 | 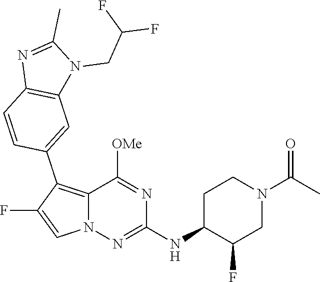 [SMILES: COc1nc(N[C@H]2CCN(C(C)=O)C[C@H]2F)nn2cc(F)c(-c3ccc4nc(C)n(CC(F)F)c4c3)c12] |
| 469 | 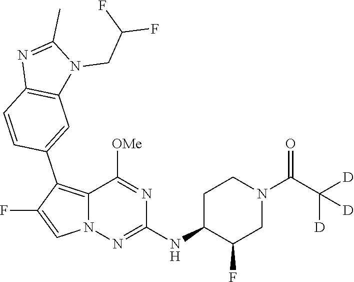 [SMILES: [2H]C([2H])([2H])C(=O)N1CC[C@H](Nc2nc(OC)c3c(-c4ccc5nc(C)n(CC(F)F)c5c4)c(F)cn3n2)[C@H](F)C1] |
| 470 | 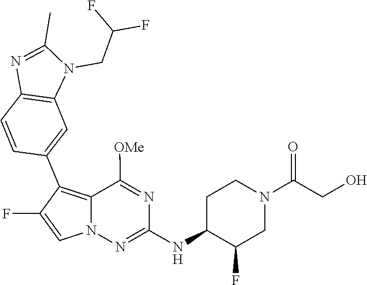 [SMILES: COc1nc(N[C@H]2CCN(C(=O)CO)C[C@H]2F)nn2cc(F)c(-c3ccc4nc(C)n(CC(F)F)c4c3)c12] |
| 471 | 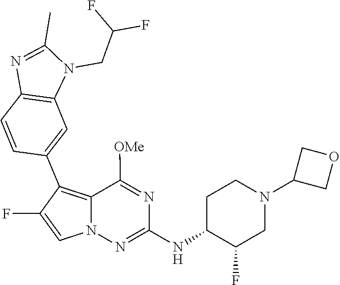 [SMILES: COc1nc(N[C@@H]2CCN(C3COC3)C[C@@H]2F)nn2cc(F)c(-c3ccc4nc(C)n(CC(F)F)c4c3)c12] |
| 472 | 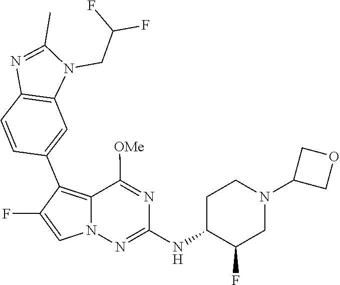 [SMILES: COc1nc(N[C@@H]2CCN(C3COC3)C[C@H]2F)nn2cc(F)c(-c3ccc4nc(C)n(CC(F)F)c4c3)c12] |
| 473 | 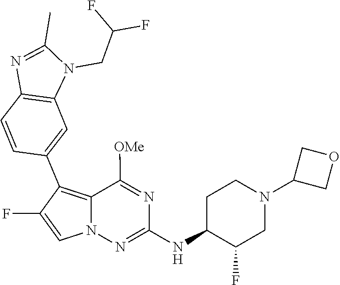 [SMILES: COc1nc(N[C@H]2CCN(C3COC3)C[C@@H]2F)nn2cc(F)c(-c3ccc4nc(C)n(CC(F)F)c4c3)c12] |
| 474 | 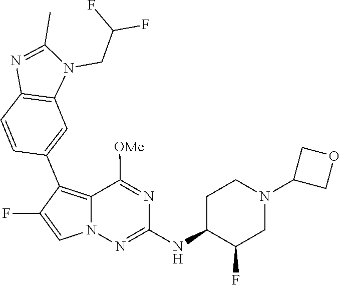 [SMILES: COc1nc(N[C@H]2CCN(C3COC3)C[C@H]2F)nn2cc(F)c(-c3ccc4nc(C)n(CC(F)F)c4c3)c12] |
| 475 | 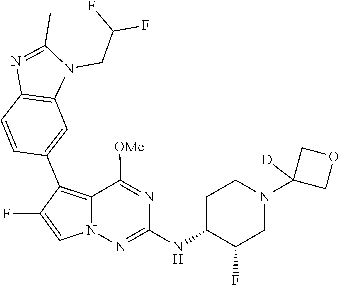 [SMILES: [2H]C1(N2CC[C@@H](Nc3nc(OC)c4c(-c5ccc6nc(C)n(CC(F)F)c6c5)c(F)cn4n3)[C@@H](F)C2)COC1] |
| 476 | 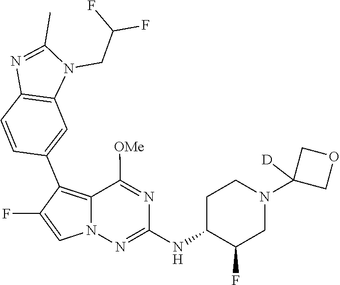 [SMILES: [2H]C1(N2CC[C@@H](Nc3nc(OC)c4c(-c5ccc6nc(C)n(CC(F)F)c6c5)c(F)cn4n3)[C@H](F)C2)COC1] |
| 477 | 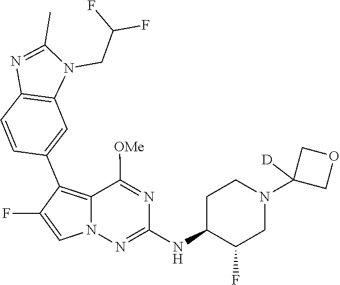 [SMILES: [2H]C1(N2CC[C@H](Nc3nc(OC)c4c(-c5ccc6nc(C)n(CC(F)F)c6c5)c(F)cn4n3)[C@@H](F)C2)COC1] |
| 478 | 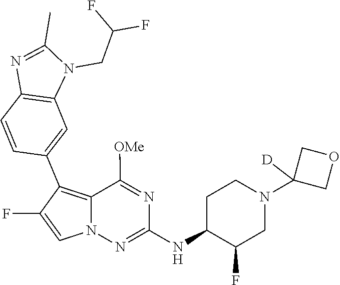 [SMILES: [2H]C1(N2CC[C@H](Nc3nc(OC)c4c(-c5ccc6nc(C)n(CC(F)F)c6c5)c(F)cn4n3)[C@H](F)C2)COC1] |
| 479 | 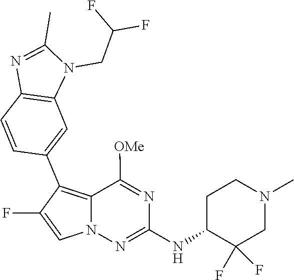 [SMILES: COc1nc(N[C@@H]2CCN(C)CC2(F)F)nn2cc(F)c(-c3ccc4nc(C)n(CC(F)F)c4c3)c12] |
| 480 | 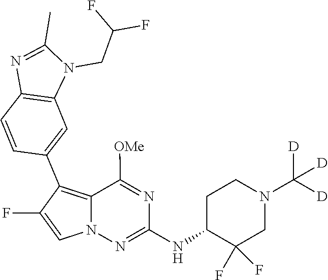 [SMILES: [2H]C([2H])([2H])N1CC[C@@H](Nc2nc(OC)c3c(-c4ccc5nc(C)n(CC(F)F)c5c4)c(F)cn3n2)C(F)(F)C1] |
| 481 | 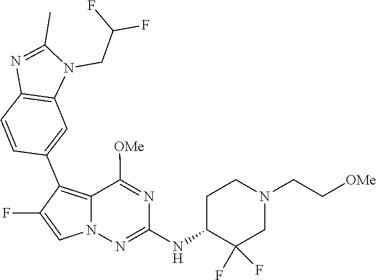 [SMILES: COCCN1CC[C@@H](Nc2nc(OC)c3c(-c4ccc5nc(C)n(CC(F)F)c5c4)c(F)cn3n2)C(F)(F)C1] |
| 482 | 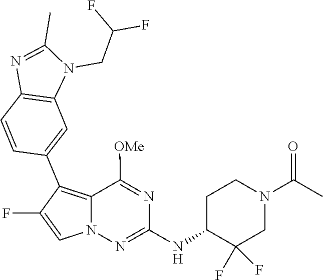 [SMILES: COc1nc(N[C@@H]2CCN(C(C)=O)CC2(F)F)nn2cc(F)c(-c3ccc4nc(C)n(CC(F)F)c4c3)c12] |
| 483 | 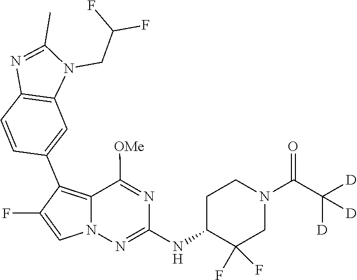 [SMILES: [2H]C([2H])([2H])C(=O)N1CC[C@@H](Nc2nc(OC)c3c(-c4ccc5nc(C)n(CC(F)F)c5c4)c(F)cn3n2)C(F)(F)C1] |
| 484 | 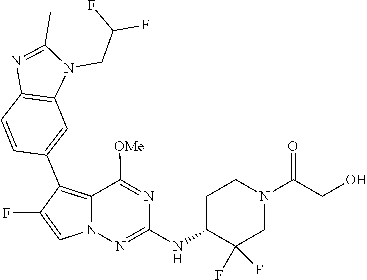 [SMILES: COc1nc(N[C@@H]2CCN(C(=O)CO)CC2(F)F)nn2cc(F)c(-c3ccc4nc(C)n(CC(F)F)c4c3)c12] |
| 485 | 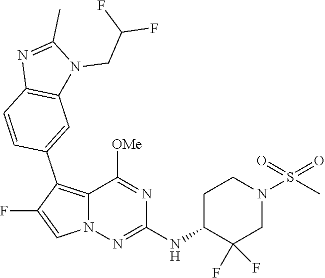 [SMILES: COc1nc(N[C@@H]2CCN(S(C)(=O)=O)CC2(F)F)nn2cc(F)c(-c3ccc4nc(C)n(CC(F)F)c4c3)c12] |
| 486 | 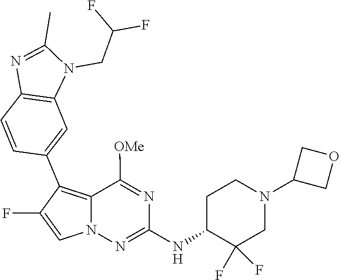 [SMILES: COc1nc(N[C@@H]2CCN(C3COC3)CC2(F)F)nn2cc(F)c(-c3ccc4nc(C)n(CC(F)F)c4c3)c12] |
| 487 | 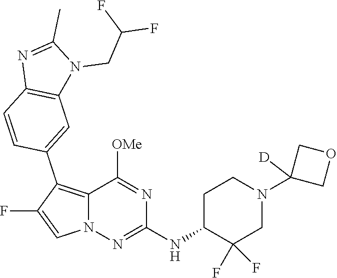 [SMILES: [2H]C1(N2CC[C@@H](Nc3nc(OC)c4c(-c5ccc6nc(C)n(CC(F)F)c6c5)c(F)cn4n3)C(F)(F)C2)COC1] |
| 488 | 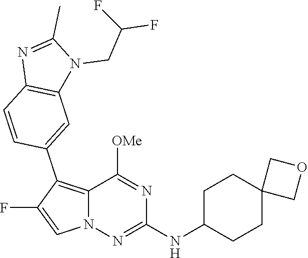 [SMILES: COc1nc(NC2CCC3(CC2)COC3)nn2cc(F)c(-c3ccc4nc(C)n(CC(F)F)c4c3)c12] |
| 489 | 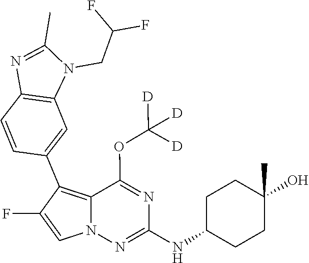 [SMILES: [2H]C([2H])([2H])Oc1nc(N[C@H]2CC[C@@](C)(O)CC2)nn2cc(F)c(-c3ccc4nc(C)n(CC(F)F)c4c3)c12] |
| 490 | 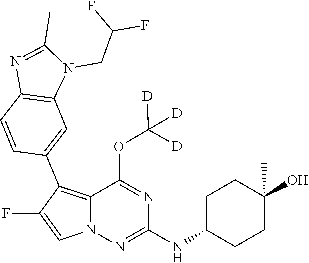 [SMILES: [2H]C([2H])([2H])Oc1nc(N[C@H]2CC[C@](C)(O)CC2)nn2cc(F)c(-c3ccc4nc(C)n(CC(F)F)c4c3)c12] |
| 491 | 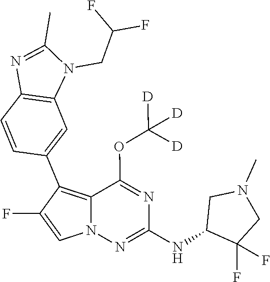 [SMILES: [2H]C([2H])([2H])Oc1nc(N[C@@H]2CN(C)CC2(F)F)nn2cc(F)c(-c3ccc4nc(C)n(CC(F)F)c4c3)c12] |
| 492 | 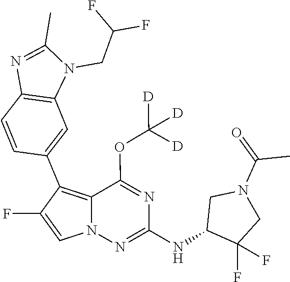 [SMILES: [2H]C([2H])([2H])Oc1nc(N[C@@H]2CN(C(C)=O)CC2(F)F)nn2cc(F)c(-c3ccc4nc(C)n(CC(F)F)c4c3)c12] |
| 493 | 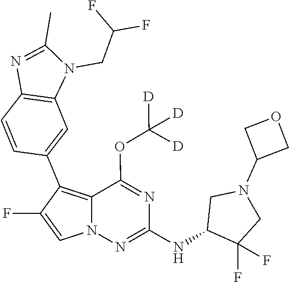 [SMILES: [2H]C([2H])([2H])Oc1nc(N[C@@H]2CN(C3COC3)CC2(F)F)nn2cc(F)c(-c3ccc4nc(C)n(CC(F)F)c4c3)c12] |
| 494 | 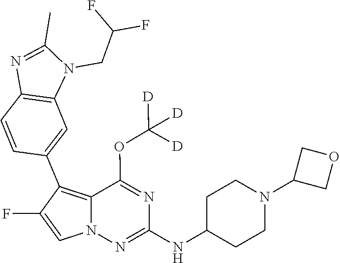 [SMILES: [2H]C([2H])([2H])Oc1nc(NC2CCN(C3COC3)CC2)nn2cc(F)c(-c3ccc4nc(C)n(CC(F)F)c4c3)c12] |
| 495 | 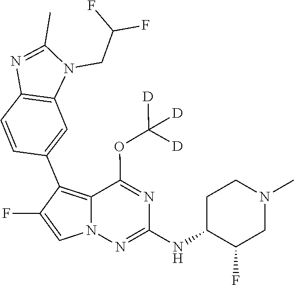 [SMILES: [2H]C([2H])([2H])Oc1nc(N[C@@H]2CCN(C)C[C@@H]2F)nn2cc(F)c(-c3ccc4nc(C)n(CC(F)F)c4c3)c12] |
| 496 | 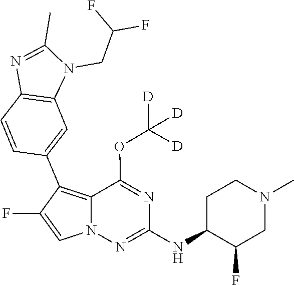 [SMILES: [2H]C([2H])([2H])Oc1nc(N[C@H]2CCN(C)C[C@H]2F)nn2cc(F)c(-c3ccc4nc(C)n(CC(F)F)c4c3)c12] |
| 497 | 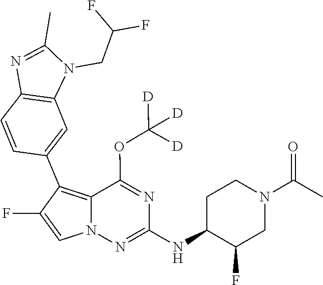 [SMILES: [2H]C([2H])([2H])Oc1nc(N[C@H]2CCN(C(C)=O)C[C@H]2F)nn2cc(F)c(-c3ccc4nc(C)n(CC(F)F)c4c3)c12] |
| 498 | 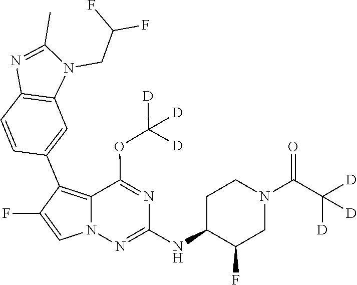 [SMILES: [2H]C([2H])([2H])Oc1nc(N[C@H]2CCN(C(=O)C([2H])([2H])[2H])C[C@H]2F)nn2cc(F)c(-c3ccc4nc(C)n(CC(F)F)c4c3)c12] |
| 499 | 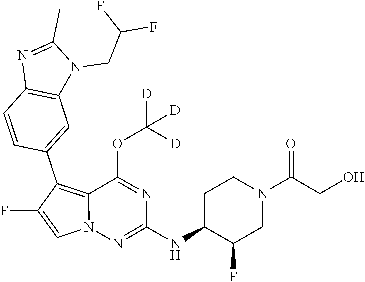 [SMILES: [2H]C([2H])([2H])Oc1nc(N[C@H]2CCN(C(=O)CO)C[C@H]2F)nn2cc(F)c(-c3ccc4nc(C)n(CC(F)F)c4c3)c12] |
| 500 | 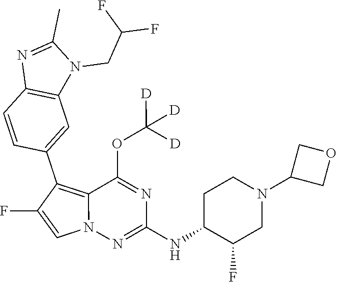 [SMILES: [2H]C([2H])([2H])Oc1nc(N[C@@H]2CCN(C3COC3)C[C@@H]2F)nn2cc(F)c(-c3ccc4nc(C)n(CC(F)F)c4c3)c12] |
| 501 | 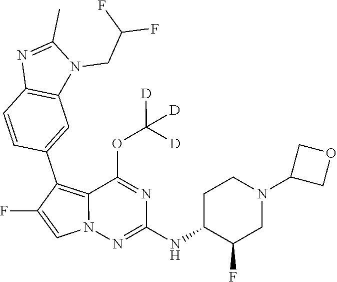 [SMILES: [2H]C([2H])([2H])Oc1nc(N[C@@H]2CCN(C3COC3)C[C@H]2F)nn2cc(F)c(-c3ccc4nc(C)n(CC(F)F)c4c3)c12] |
| 502 | 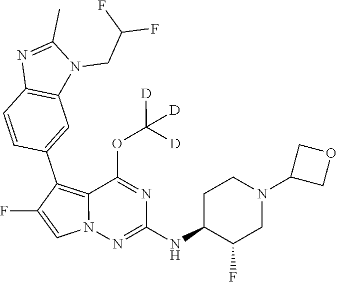 [SMILES: [2H]C([2H])([2H])Oc1nc(N[C@H]2CCN(C3COC3)C[C@@H]2F)nn2cc(F)c(-c3ccc4nc(C)n(CC(F)F)c4c3)c12] |
| 503 | 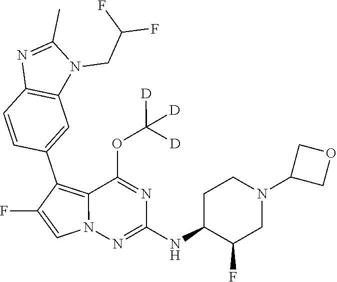 [SMILES: [2H]C([2H])([2H])Oc1nc(N[C@H]2CCN(C3COC3)C[C@H]2F)nn2cc(F)c(-c3ccc4nc(C)n(CC(F)F)c4c3)c12] |
| 504 | 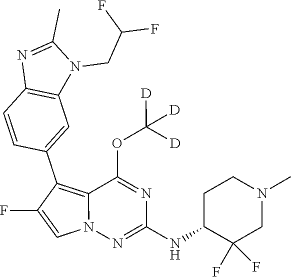 [SMILES: [2H]C([2H])([2H])Oc1nc(N[C@@H]2CCN(C)CC2(F)F)nn2cc(F)c(-c3ccc4nc(C)n(CC(F)F)c4c3)c12] |
| 505 | 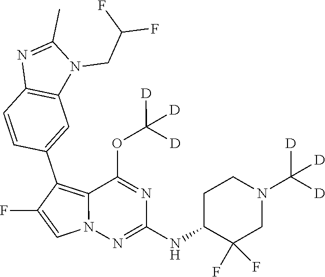 [SMILES: [2H]C([2H])([2H])Oc1nc(N[C@@H]2CCN(C([2H])([2H])[2H])CC2(F)F)nn2cc(F)c(-c3ccc4nc(C)n(CC(F)F)c4c3)c12] |
| 506 | 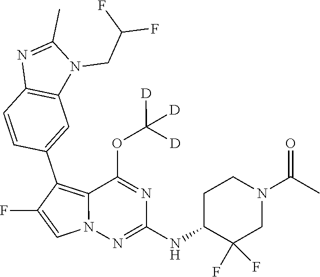 [SMILES: [2H]C([2H])([2H])Oc1nc(N[C@@H]2CCN(C(C)=O)CC2(F)F)nn2cc(F)c(-c3ccc4nc(C)n(CC(F)F)c4c3)c12] |
| 507 | 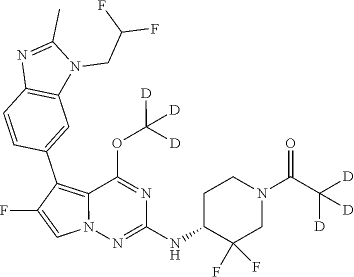 [SMILES: [2H]C([2H])([2H])Oc1nc(N[C@@H]2CCN(C(=O)C([2H])([2H])[2H])CC2(F)F)nn2cc(F)c(-c3ccc4nc(C)n(CC(F)F)c4c3)c12] |
| 508 | 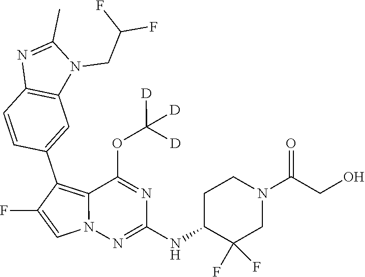 [SMILES: [2H]C([2H])([2H])Oc1nc(N[C@@H]2CCN(C(=O)CO)CC2(F)F)nn2cc(F)c(-c3ccc4nc(C)n(CC(F)F)c4c3)c12] |
| 509 | 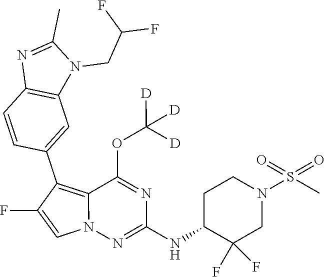 [SMILES: [2H]C([2H])([2H])Oc1nc(N[C@@H]2CCN(S(C)(=O)=O)CC2(F)F)nn2cc(F)c(-c3ccc4nc(C)n(CC(F)F)c4c3)c12] |
| 510 | 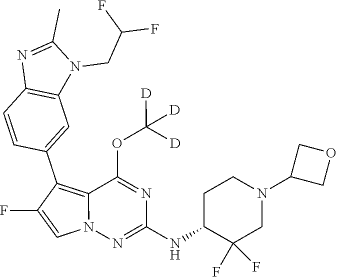 [SMILES: [2H]C([2H])([2H])Oc1nc(N[C@@H]2CCN(C3COC3)CC2(F)F)nn2cc(F)c(-c3ccc4nc(C)n(CC(F)F)c4c3)c12] |
| 511 | 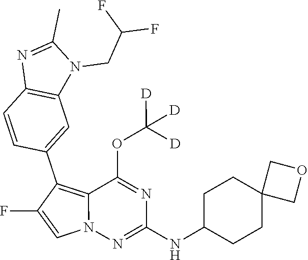 [SMILES: [2H]C([2H])([2H])Oc1nc(NC2CCC3(CC2)COC3)nn2cc(F)c(-c3ccc4nc(C)n(CC(F)F)c4c3)c12] |
| 512 | 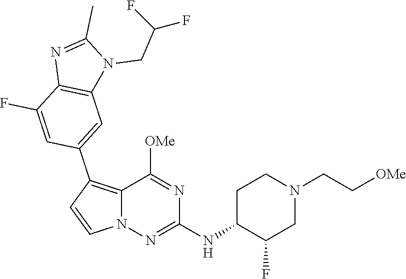 [SMILES: COCCN1CC[C@@H](Nc2nc(OC)c3c(-c4cc(F)c5nc(C)n(CC(F)F)c5c4)ccn3n2)[C@@H](F)C1] |
| 513 | 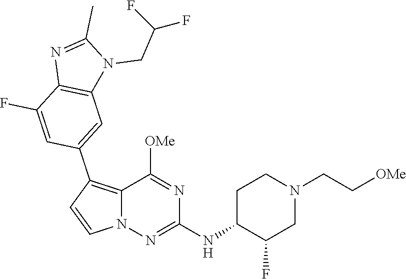 [SMILES: COCCN1CC[C@@H](Nc2nc(OC)c3c(-c4cc(F)c5nc(C)n(CC(F)F)c5c4)ccn3n2)[C@@H](F)C1] |
| 514 | 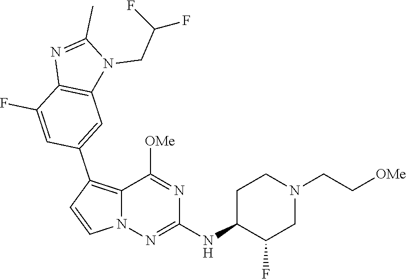 [SMILES: COCCN1CC[C@H](Nc2nc(OC)c3c(-c4cc(F)c5nc(C)n(CC(F)F)c5c4)ccn3n2)[C@@H](F)C1] |
| 515 | 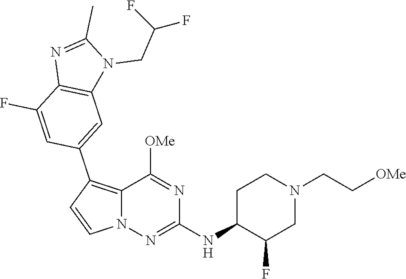 [SMILES: COCCN1CC[C@H](Nc2nc(OC)c3c(-c4cc(F)c5nc(C)n(CC(F)F)c5c4)ccn3n2)[C@H](F)C1] |
| 516 | 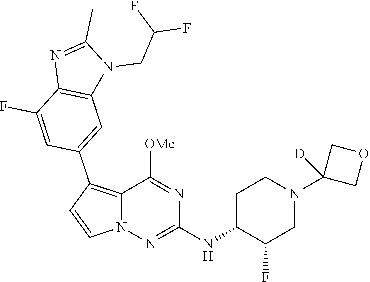 [SMILES: [2H]C1(N2CC[C@@H](Nc3nc(OC)c4c(-c5cc(F)c6nc(C)n(CC(F)F)c6c5)ccn4n3)[C@@H](F)C2)COC1] |
| 517 | 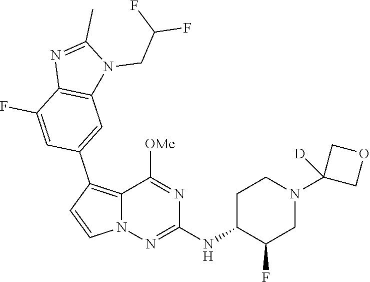 [SMILES: [2H]C1(N2CC[C@@H](Nc3nc(OC)c4c(-c5cc(F)c6nc(C)n(CC(F)F)c6c5)ccn4n3)[C@H](F)C2)COC1] |
| 518 | 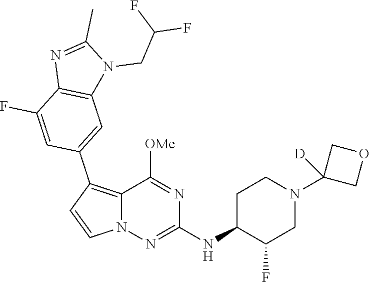 [SMILES: [2H]C1(N2CC[C@H](Nc3nc(OC)c4c(-c5cc(F)c6nc(C)n(CC(F)F)c6c5)ccn4n3)[C@@H](F)C2)COC1] |
| 519 | 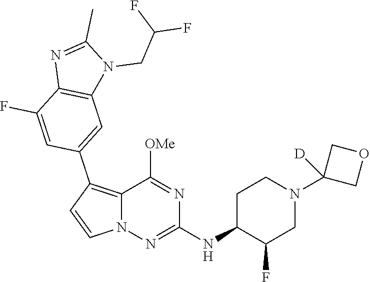 [SMILES: [2H]C1(N2CC[C@H](Nc3nc(OC)c4c(-c5cc(F)c6nc(C)n(CC(F)F)c6c5)ccn4n3)[C@H](F)C2)COC1] |
| 520 | 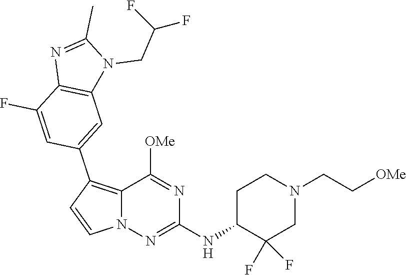 [SMILES: COCCN1CC[C@@H](Nc2nc(OC)c3c(-c4cc(F)c5nc(C)n(CC(F)F)c5c4)ccn3n2)C(F)(F)C1] |
| 521 | 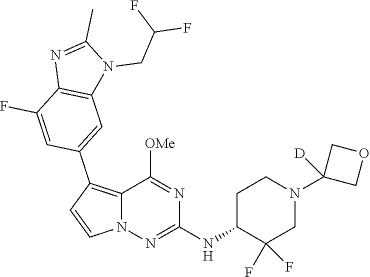 [SMILES: [2H]C1(N2CC[C@@H](Nc3nc(OC)c4c(-c5cc(F)c6nc(C)n(CC(F)F)c6c5)ccn4n3)C(F)(F)C2)COC1] |
| 522 | 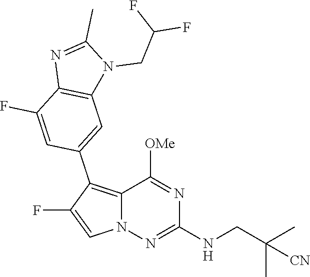 [SMILES: COc1nc(NCC(C)(C)C#N)nn2cc(F)c(-c3cc(F)c4nc(C)n(CC(F)F)c4c3)c12] |
| 523 | 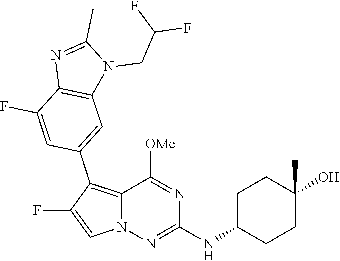 [SMILES: COc1nc(N[C@H]2CC[C@@](C)(O)CC2)nn2cc(F)c(-c3cc(F)c4nc(C)n(CC(F)F)c4c3)c12] |
| 524 | 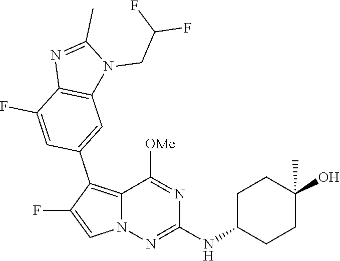 [SMILES: COc1nc(N[C@H]2CC[C@](C)(O)CC2)nn2cc(F)c(-c3cc(F)c4nc(C)n(CC(F)F)c4c3)c12] |
| 525 | 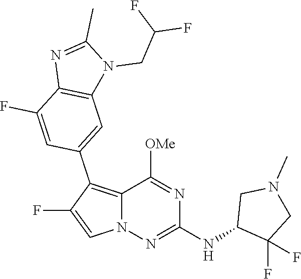 [SMILES: COc1nc(N[C@@H]2CN(C)CC2(F)F)nn2cc(F)c(-c3cc(F)c4nc(C)n(CC(F)F)c4c3)c12] |
| 526 | 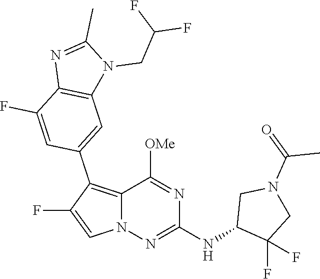 [SMILES: COc1nc(N[C@@H]2CN(C(C)=O)CC2(F)F)nn2cc(F)c(-c3cc(F)c4nc(C)n(CC(F)F)c4c3)c12] |
| 527 | 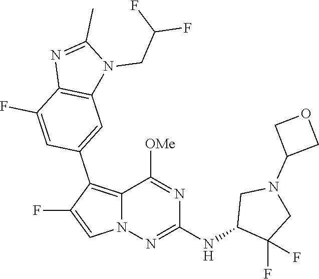 [SMILES: COc1nc(N[C@@H]2CN(C3COC3)CC2(F)F)nn2cc(F)c(-c3cc(F)c4nc(C)n(CC(F)F)c4c3)c12] |
| 528 | 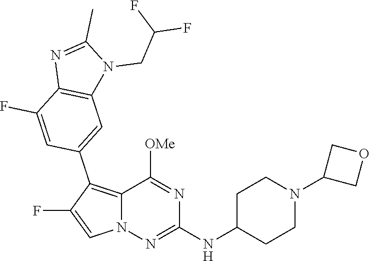 [SMILES: COc1nc(NC2CCN(C3COC3)CC2)nn2cc(F)c(-c3cc(F)c4nc(C)n(CC(F)F)c4c3)c12] |
| 529 | 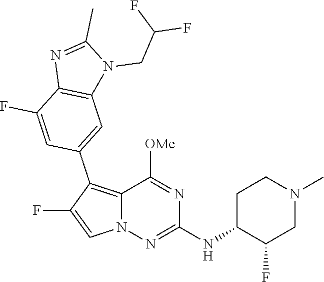 [SMILES: COc1nc(N[C@@H]2CCN(C)C[C@@H]2F)nn2cc(F)c(-c3cc(F)c4nc(C)n(CC(F)F)c4c3)c12] |
| 530 | 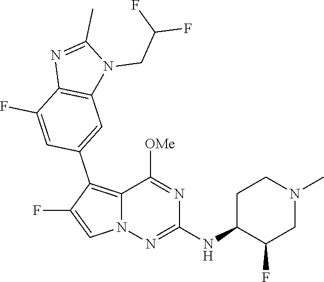 [SMILES: COc1nc(N[C@H]2CCN(C)C[C@H]2F)nn2cc(F)c(-c3cc(F)c4nc(C)n(CC(F)F)c4c3)c12] |
| 531 | 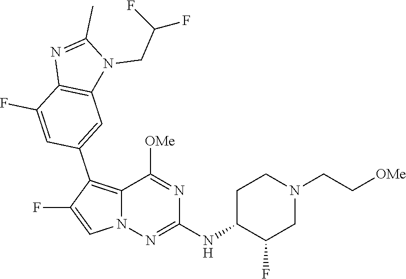 [SMILES: COCCN1CC[C@@H](Nc2nc(OC)c3c(-c4cc(F)c5nc(C)n(CC(F)F)c5c4)c(F)cn3n2)[C@@H](F)C1] |
| 532 | 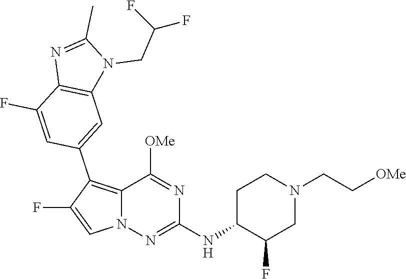 [SMILES: COCCN1CC[C@@H](Nc2nc(OC)c3c(-c4cc(F)c5nc(C)n(CC(F)F)c5c4)c(F)cn3n2)[C@H](F)C1] |
| 533 | 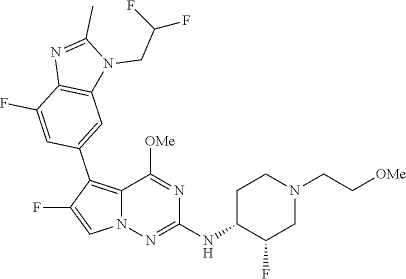 [SMILES: COCCN1CC[C@@H](Nc2nc(OC)c3c(-c4cc(F)c5nc(C)n(CC(F)F)c5c4)c(F)cn3n2)[C@@H](F)C1] |
| 534 | 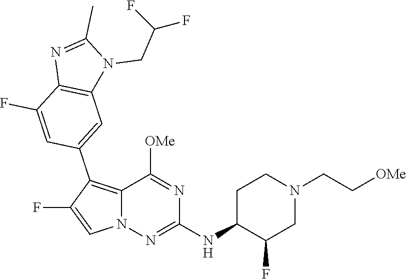 [SMILES: COCCN1CC[C@H](Nc2nc(OC)c3c(-c4cc(F)c5nc(C)n(CC(F)F)c5c4)c(F)cn3n2)[C@H](F)C1] |
| 535 | 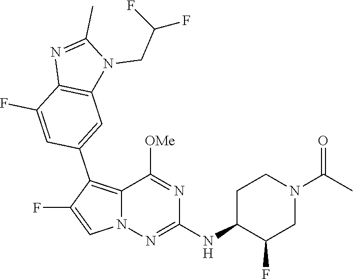 [SMILES: COc1nc(N[C@H]2CCN(C(C)=O)C[C@H]2F)nn2cc(F)c(-c3cc(F)c4nc(C)n(CC(F)F)c4c3)c12] |
| 536 | 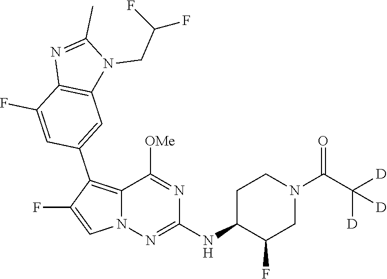 [SMILES: [2H]C([2H])([2H])C(=O)N1CC[C@H](Nc2nc(OC)c3c(-c4cc(F)c5nc(C)n(CC(F)F)c5c4)c(F)cn3n2)[C@H](F)C1] |
| 537 | 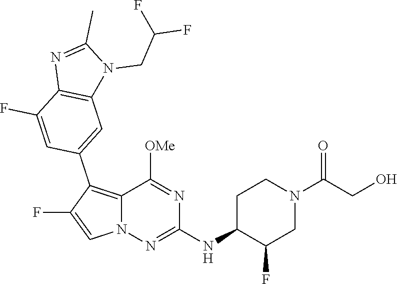 [SMILES: COc1nc(N[C@H]2CCN(C(=O)CO)C[C@H]2F)nn2cc(F)c(-c3cc(F)c4nc(C)n(CC(F)F)c4c3)c12] |
| 538 | 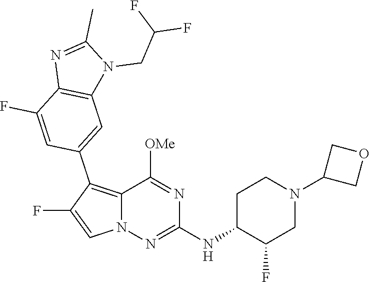 [SMILES: COc1nc(N[C@@H]2CCN(C3COC3)C[C@@H]2F)nn2cc(F)c(-c3cc(F)c4nc(C)n(CC(F)F)c4c3)c12] |
| 539 | 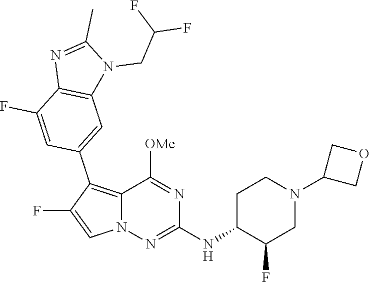 [SMILES: COc1nc(N[C@@H]2CCN(C3COC3)C[C@H]2F)nn2cc(F)c(-c3cc(F)c4nc(C)n(CC(F)F)c4c3)c12] |
| 540 | 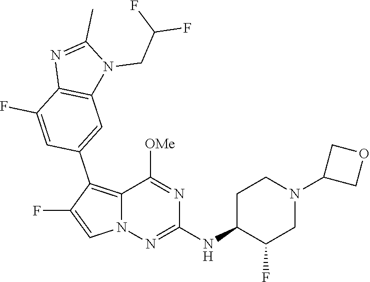 [SMILES: COc1nc(N[C@H]2CCN(C3COC3)C[C@@H]2F)nn2cc(F)c(-c3cc(F)c4nc(C)n(CC(F)F)c4c3)c12] |
| 541 | 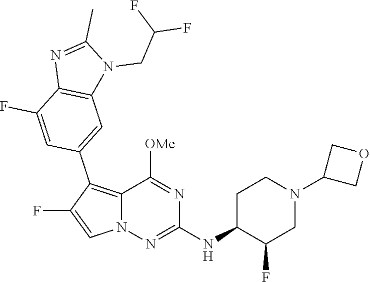 [SMILES: COc1nc(N[C@H]2CCN(C3COC3)C[C@H]2F)nn2cc(F)c(-c3cc(F)c4nc(C)n(CC(F)F)c4c3)c12] |
| 542 | 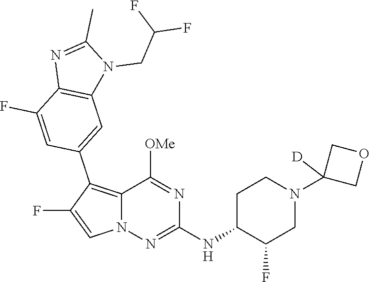 [SMILES: [2H]C1(N2CC[C@@H](Nc3nc(OC)c4c(-c5cc(F)c6nc(C)n(CC(F)F)c6c5)c(F)cn4n3)[C@@H](F)C2)COC1] |
| 543 | 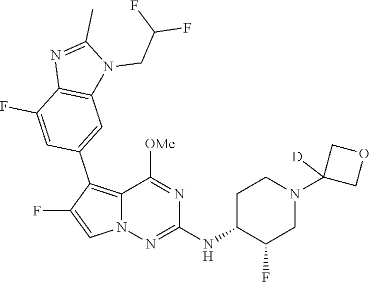 [SMILES: [2H]C1(N2CC[C@@H](Nc3nc(OC)c4c(-c5cc(F)c6nc(C)n(CC(F)F)c6c5)c(F)cn4n3)[C@@H](F)C2)COC1] |
| 544 | 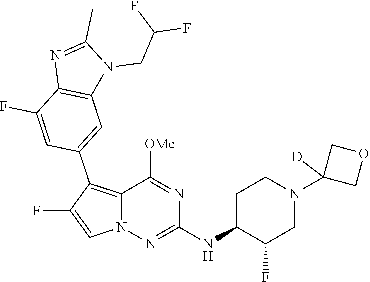 [SMILES: [2H]C1(N2CC[C@H](Nc3nc(OC)c4c(-c5cc(F)c6nc(C)n(CC(F)F)c6c5)c(F)cn4n3)[C@@H](F)C2)COC1] |
| 545 | 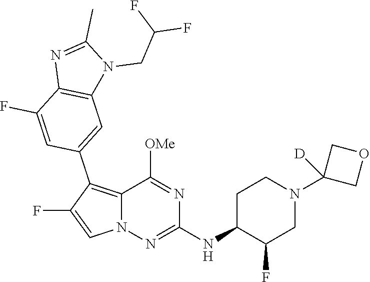 [SMILES: [2H]C1(N2CC[C@H](Nc3nc(OC)c4c(-c5cc(F)c6nc(C)n(CC(F)F)c6c5)c(F)cn4n3)[C@H](F)C2)COC1] |
| 546 | 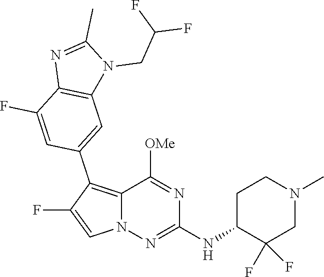 [SMILES: COc1nc(N[C@@H]2CCN(C)CC2(F)F)nn2cc(F)c(-c3cc(F)c4nc(C)n(CC(F)F)c4c3)c12] |
| 547 | 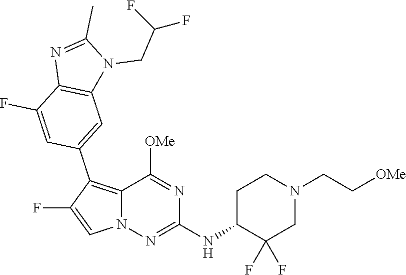 [SMILES: COCCN1CC[C@@H](Nc2nc(OC)c3c(-c4cc(F)c5nc(C)n(CC(F)F)c5c4)c(F)cn3n2)C(F)(F)C1] |
| 548 | 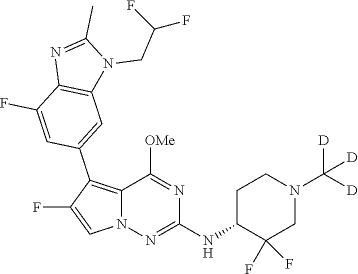 [SMILES: [2H]C([2H])([2H])N1CC[C@@H](Nc2nc(OC)c3c(-c4cc(F)c5nc(C)n(CC(F)F)c5c4)c(F)cn3n2)C(F)(F)C1] |
| 549 | 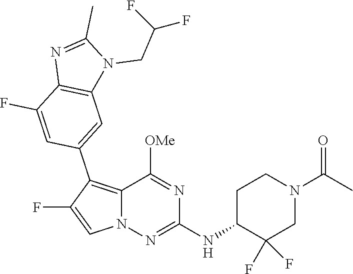 [SMILES: COc1nc(N[C@@H]2CCN(C(C)=O)CC2(F)F)nn2cc(F)c(-c3cc(F)c4nc(C)n(CC(F)F)c4c3)c12] |
| 550 | 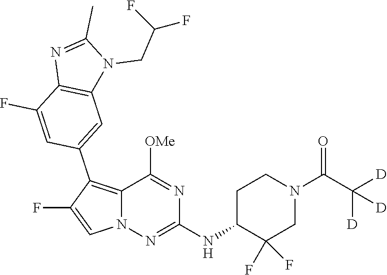 [SMILES: [2H]C([2H])([2H])C(=O)N1CC[C@@H](Nc2nc(OC)c3c(-c4cc(F)c5nc(C)n(CC(F)F)c5c4)c(F)cn3n2)C(F)(F)C1] |
| 551 | 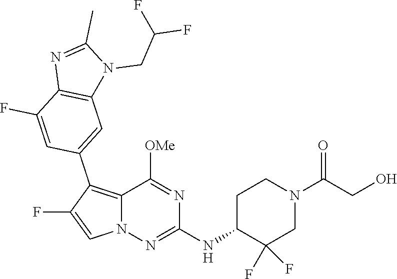 [SMILES: COc1nc(N[C@@H]2CCN(C(=O)CO)CC2(F)F)nn2cc(F)c(-c3cc(F)c4nc(C)n(CC(F)F)c4c3)c12] |
| 552 | 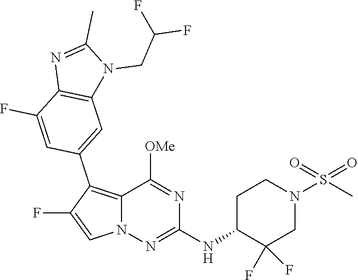 [SMILES: COc1nc(N[C@@H]2CCN(S(C)(=O)=O)CC2(F)F)nn2cc(F)c(-c3cc(F)c4nc(C)n(CC(F)F)c4c3)c12] |
| 553 | 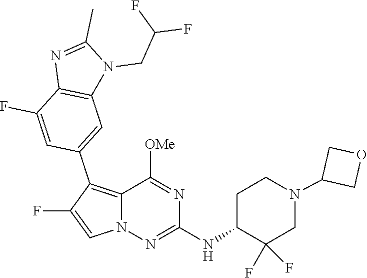 [SMILES: COc1nc(N[C@@H]2CCN(C3COC3)CC2(F)F)nn2cc(F)c(-c3cc(F)c4nc(C)n(CC(F)F)c4c3)c12] |
| 554 | 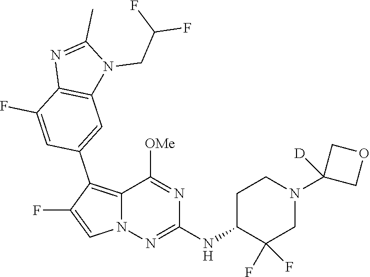 [SMILES: [2H]C1(N2CC[C@@H](Nc3nc(OC)c4c(-c5cc(F)c6nc(C)n(CC(F)F)c6c5)c(F)cn4n3)C(F)(F)C2)COC1] |
| 555 | 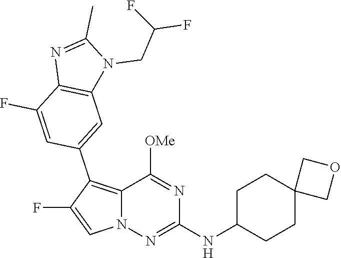 [SMILES: COc1nc(NC2CCC3(CC2)COC3)nn2cc(F)c(-c3cc(F)c4nc(C)n(CC(F)F)c4c3)c12] |
| 556 | 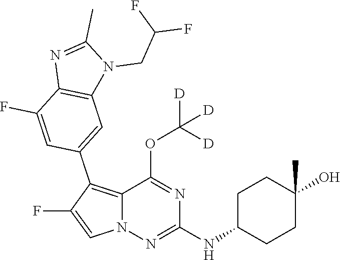 [SMILES: [2H]C([2H])([2H])Oc1nc(N[C@H]2CC[C@@](C)(O)CC2)nn2cc(F)c(-c3cc(F)c4nc(C)n(CC(F)F)c4c3)c12] |
| 557 | 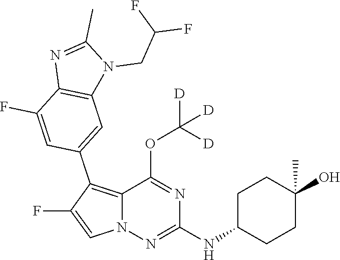 [SMILES: [2H]C([2H])([2H])Oc1nc(N[C@H]2CC[C@](C)(O)CC2)nn2cc(F)c(-c3cc(F)c4nc(C)n(CC(F)F)c4c3)c12] |
| 558 | 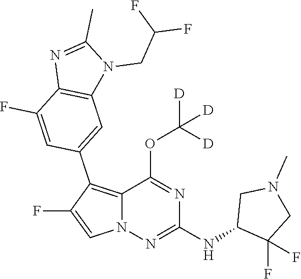 [SMILES: [2H]C([2H])([2H])Oc1nc(N[C@@H]2CN(C)CC2(F)F)nn2cc(F)c(-c3cc(F)c4nc(C)n(CC(F)F)c4c3)c12] |
| 559 | 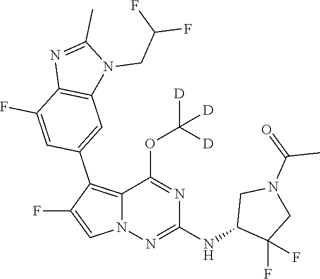 [SMILES: [2H]C([2H])([2H])Oc1nc(N[C@@H]2CN(C(C)=O)CC2(F)F)nn2cc(F)c(-c3cc(F)c4nc(C)n(CC(F)F)c4c3)c12] |
| 560 | 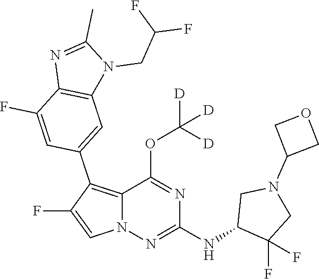 [SMILES: [2H]C([2H])([2H])Oc1nc(N[C@@H]2CN(C3COC3)CC2(F)F)nn2cc(F)c(-c3cc(F)c4nc(C)n(CC(F)F)c4c3)c12] |
| 561 | 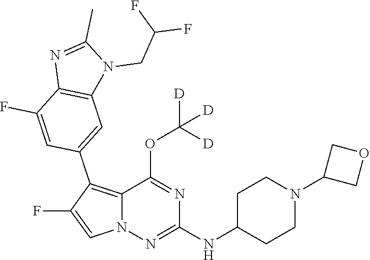 [SMILES: [2H]C([2H])([2H])Oc1nc(NC2CCN(C3COC3)CC2)nn2cc(F)c(-c3cc(F)c4nc(C)n(CC(F)F)c4c3)c12] |
| 562 | 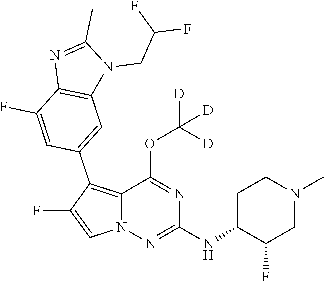 [SMILES: [2H]C([2H])([2H])Oc1nc(N[C@@H]2CCN(C)C[C@@H]2F)nn2cc(F)c(-c3cc(F)c4nc(C)n(CC(F)F)c4c3)c12] |
| 563 | 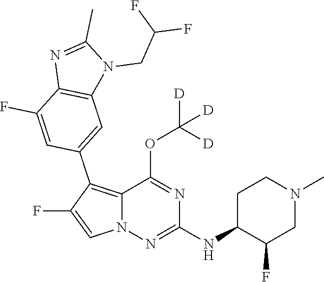 [SMILES: [2H]C([2H])([2H])Oc1nc(N[C@H]2CCN(C)C[C@H]2F)nn2cc(F)c(-c3cc(F)c4nc(C)n(CC(F)F)c4c3)c12] |
| 564 | 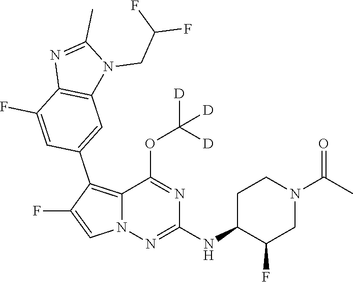 [SMILES: [2H]C([2H])([2H])Oc1nc(N[C@H]2CCN(C(C)=O)C[C@H]2F)nn2cc(F)c(-c3cc(F)c4nc(C)n(CC(F)F)c4c3)c12] |
| 565 | 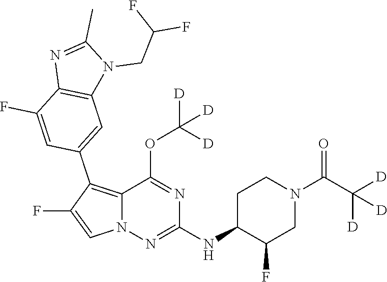 [SMILES: [2H]C([2H])([2H])Oc1nc(N[C@H]2CCN(C(=O)C([2H])([2H])[2H])C[C@H]2F)nn2cc(F)c(-c3cc(F)c4nc(C)n(CC(F)F)c4c3)c12] |
| 566 | 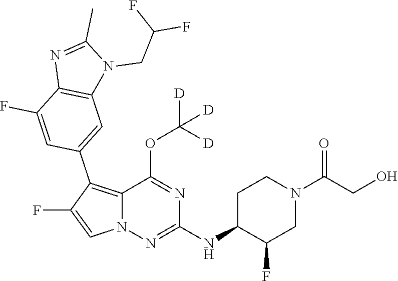 [SMILES: [2H]C([2H])([2H])Oc1nc(N[C@H]2CCN(C(=O)CO)C[C@H]2F)nn2cc(F)c(-c3cc(F)c4nc(C)n(CC(F)F)c4c3)c12] |
| 567 | 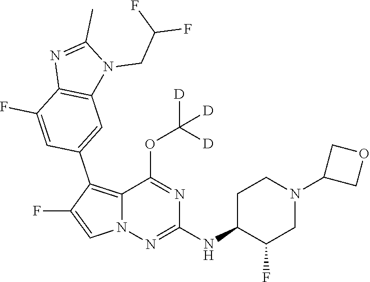 [SMILES: [2H]C([2H])([2H])Oc1nc(N[C@H]2CCN(C3COC3)C[C@@H]2F)nn2cc(F)c(-c3cc(F)c4nc(C)n(CC(F)F)c4c3)c12] |
| 568 | 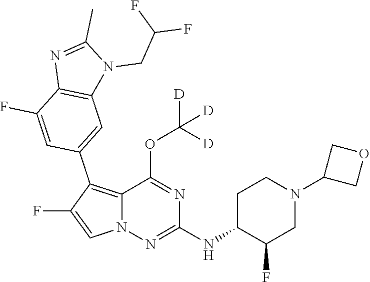 [SMILES: [2H]C([2H])([2H])Oc1nc(N[C@@H]2CCN(C3COC3)C[C@H]2F)nn2cc(F)c(-c3cc(F)c4nc(C)n(CC(F)F)c4c3)c12] |
| 569 | 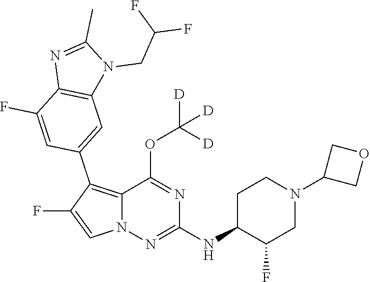 [SMILES: [2H]C([2H])([2H])Oc1nc(N[C@H]2CCN(C3COC3)C[C@@H]2F)nn2cc(F)c(-c3cc(F)c4nc(C)n(CC(F)F)c4c3)c12] |
| 570 | 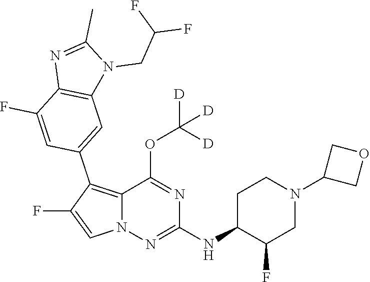 [SMILES: [2H]C([2H])([2H])Oc1nc(N[C@H]2CCN(C3COC3)C[C@H]2F)nn2cc(F)c(-c3cc(F)c4nc(C)n(CC(F)F)c4c3)c12] |
| 571 | 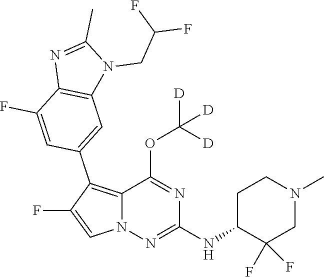 [SMILES: [2H]C([2H])([2H])Oc1nc(N[C@@H]2CCN(C)CC2(F)F)nn2cc(F)c(-c3cc(F)c4nc(C)n(CC(F)F)c4c3)c12] |
| 572 | 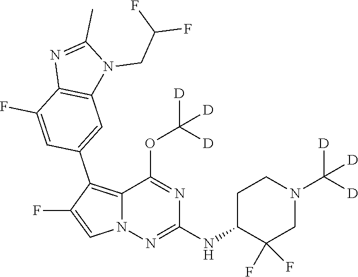 [SMILES: [2H]C([2H])([2H])Oc1nc(N[C@@H]2CCN(C([2H])([2H])[2H])CC2(F)F)nn2cc(F)c(-c3cc(F)c4nc(C)n(CC(F)F)c4c3)c12] |
| 573 | 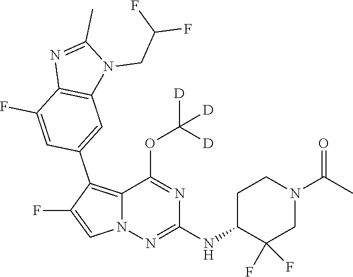 [SMILES: [2H]C([2H])([2H])Oc1nc(N[C@@H]2CCN(C(C)=O)CC2(F)F)nn2cc(F)c(-c3cc(F)c4nc(C)n(CC(F)F)c4c3)c12] |
| 574 | 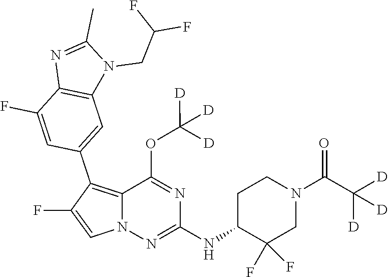 [SMILES: [2H]C([2H])([2H])Oc1nc(N[C@@H]2CCN(C(=O)C([2H])([2H])[2H])CC2(F)F)nn2cc(F)c(-c3cc(F)c4nc(C)n(CC(F)F)c4c3)c12] |
| 575 | 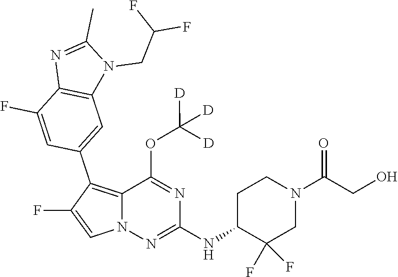 [SMILES: [2H]C([2H])([2H])Oc1nc(N[C@@H]2CCN(C(=O)CO)CC2(F)F)nn2cc(F)c(-c3cc(F)c4nc(C)n(CC(F)F)c4c3)c12] |
| 576 | 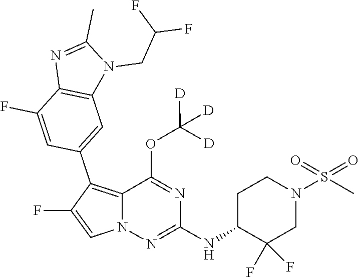 [SMILES: [2H]C([2H])([2H])Oc1nc(N[C@@H]2CCN(S(C)(=O)=O)CC2(F)F)nn2cc(F)c(-c3cc(F)c4nc(C)n(CC(F)F)c4c3)c12] |
| 577 | 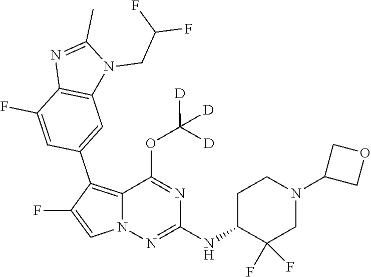 [SMILES: [2H]C([2H])([2H])Oc1nc(N[C@@H]2CCN(C3COC3)CC2(F)F)nn2cc(F)c(-c3cc(F)c4nc(C)n(CC(F)F)c4c3)c12] |
| 578 | 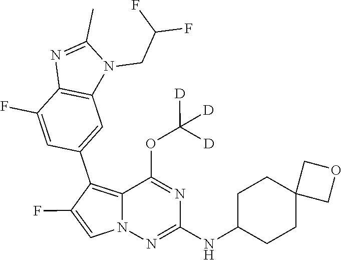 [SMILES: [2H]C([2H])([2H])Oc1nc(NC2CCC3(CC2)COC3)nn2cc(F)c(-c3cc(F)c4nc(C)n(CC(F)F)c4c3)c12] |
| 579 | 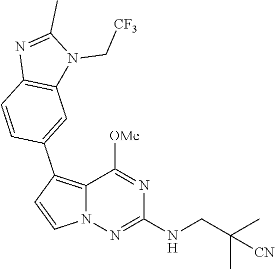 [SMILES: COc1nc(NCC(C)(C)C#N)nn2ccc(-c3ccc4nc(C)n(CC(F)(F)F)c4c3)c12] |
| 580 | 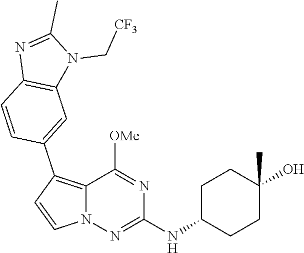 [SMILES: COc1nc(N[C@H]2CC[C@@](C)(O)CC2)nn2ccc(-c3ccc4nc(C)n(CC(F)(F)F)c4c3)c12] |
| 581 | 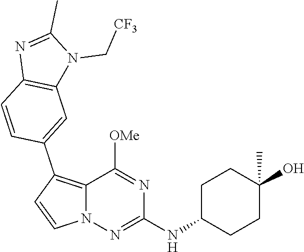 [SMILES: COc1nc(N[C@H]2CC[C@](C)(O)CC2)nn2ccc(-c3ccc4nc(C)n(CC(F)(F)F)c4c3)c12] |
| 582 | 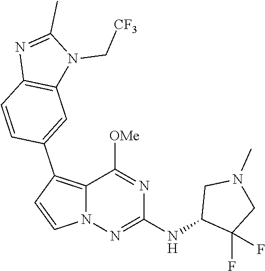 [SMILES: COc1nc(N[C@@H]2CN(C)CC2(F)F)nn2ccc(-c3ccc4nc(C)n(CC(F)(F)F)c4c3)c12] |
| 583 | 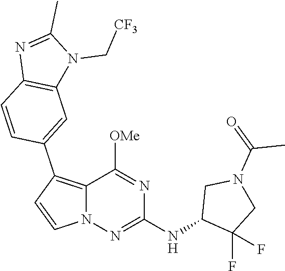 [SMILES: COc1nc(N[C@@H]2CN(C(C)=O)CC2(F)F)nn2ccc(-c3ccc4nc(C)n(CC(F)(F)F)c4c3)c12] |
| 584 | 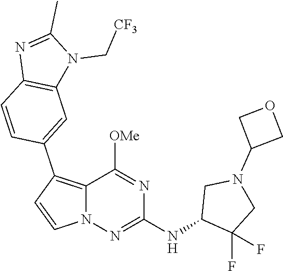 [SMILES: COc1nc(N[C@@H]2CN(C3COC3)CC2(F)F)nn2ccc(-c3ccc4nc(C)n(CC(F)(F)F)c4c3)c12] |
| 585 | 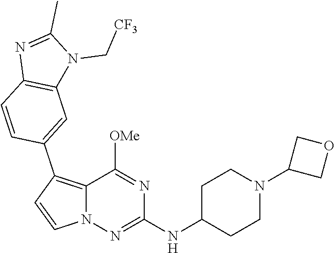 [SMILES: COc1nc(NC2CCN(C3COC3)CC2)nn2ccc(-c3ccc4nc(C)n(CC(F)(F)F)c4c3)c12] |
| 586 | 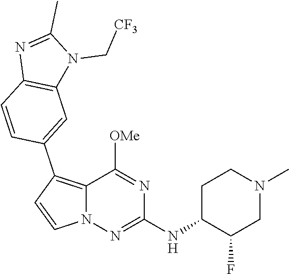 [SMILES: COc1nc(N[C@@H]2CCN(C)C[C@@H]2F)nn2ccc(-c3ccc4nc(C)n(CC(F)(F)F)c4c3)c12] |
| 587 | 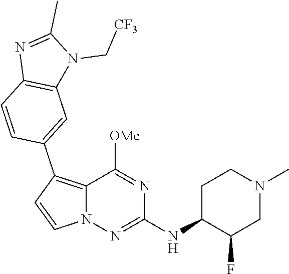 [SMILES: COc1nc(N[C@H]2CCN(C)C[C@H]2F)nn2ccc(-c3ccc4nc(C)n(CC(F)(F)F)c4c3)c12] |
| 588 | 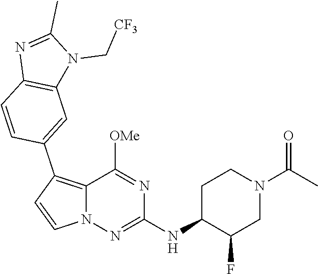 [SMILES: COc1nc(N[C@H]2CCN(C(C)=O)C[C@H]2F)nn2ccc(-c3ccc4nc(C)n(CC(F)(F)F)c4c3)c12] |
| 589 | 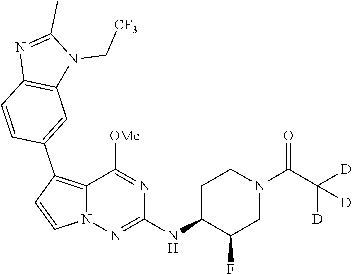 [SMILES: [2H]C([2H])([2H])C(=O)N1CC[C@H](Nc2nc(OC)c3c(-c4ccc5nc(C)n(CC(F)(F)F)c5c4)ccn3n2)[C@H](F)C1] |
| 590 | 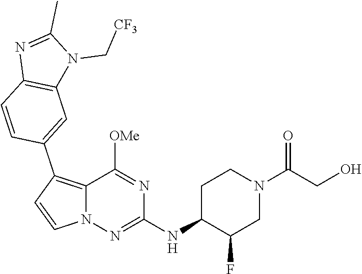 [SMILES: COc1nc(N[C@H]2CCN(C(=O)CO)C[C@H]2F)nn2ccc(-c3ccc4nc(C)n(CC(F)(F)F)c4c3)c12] |
| 591 | 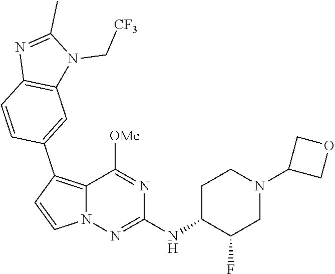 [SMILES: COc1nc(N[C@@H]2CCN(C3COC3)C[C@@H]2F)nn2ccc(-c3ccc4nc(C)n(CC(F)(F)F)c4c3)c12] |
| 592 | 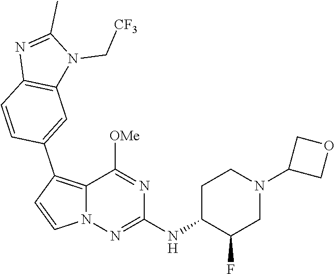 [SMILES: COc1nc(N[C@@H]2CCN(C3COC3)C[C@H]2F)nn2ccc(-c3ccc4nc(C)n(CC(F)(F)F)c4c3)c12] |
| 593 | 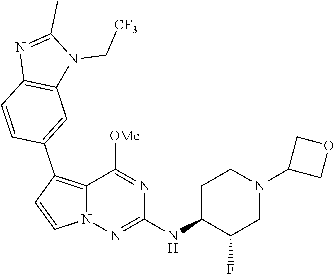 [SMILES: COc1nc(N[C@H]2CCN(C3COC3)C[C@@H]2F)nn2ccc(-c3ccc4nc(C)n(CC(F)(F)F)c4c3)c12] |
| 594 | 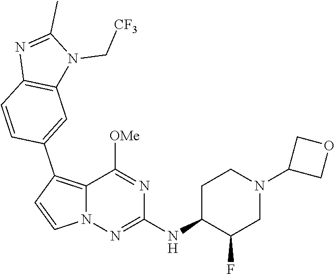 [SMILES: COc1nc(N[C@H]2CCN(C3COC3)C[C@H]2F)nn2ccc(-c3ccc4nc(C)n(CC(F)(F)F)c4c3)c12] |
| 595 | 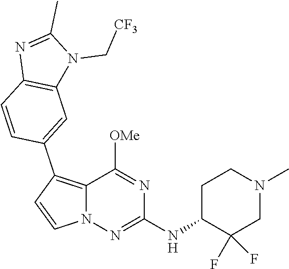 [SMILES: COc1nc(N[C@@H]2CCN(C)CC2(F)F)nn2ccc(-c3ccc4nc(C)n(CC(F)(F)F)c4c3)c12] |
| 596 | 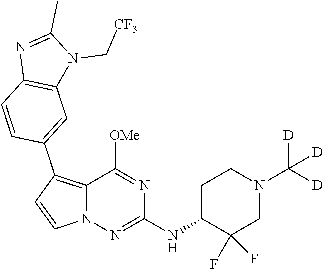 [SMILES: [2H]C([2H])([2H])N1CC[C@@H](Nc2nc(OC)c3c(-c4ccc5nc(C)n(CC(F)(F)F)c5c4)ccn3n2)C(F)(F)C1] |
| 597 | 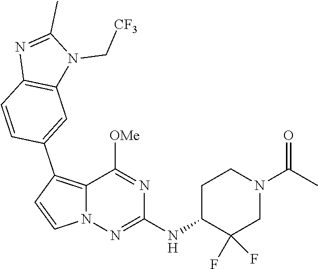 [SMILES: COc1nc(N[C@@H]2CCN(C(C)=O)CC2(F)F)nn2ccc(-c3ccc4nc(C)n(CC(F)(F)F)c4c3)c12] |
| 598 | 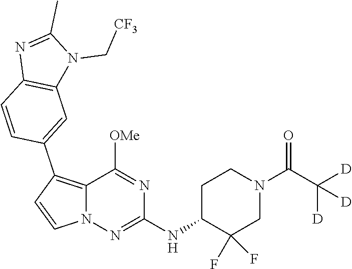 [SMILES: [2H]C([2H])([2H])C(=O)N1CC[C@@H](Nc2nc(OC)c3c(-c4ccc5nc(C)n(CC(F)(F)F)c5c4)ccn3n2)C(F)(F)C1] |
| 599 | 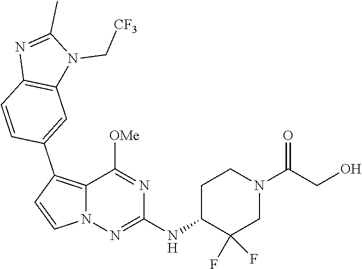 [SMILES: COc1nc(N[C@@H]2CCN(C(=O)CO)CC2(F)F)nn2ccc(-c3ccc4nc(C)n(CC(F)(F)F)c4c3)c12] |
| 600 | 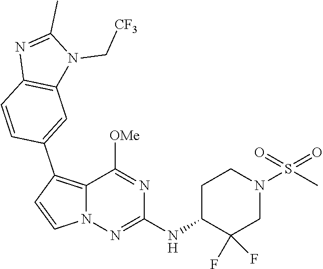 [SMILES: COc1nc(N[C@@H]2CCN(S(C)(=O)=O)CC2(F)F)nn2ccc(-c3ccc4nc(C)n(CC(F)(F)F)c4c3)c12] |
| 601 | 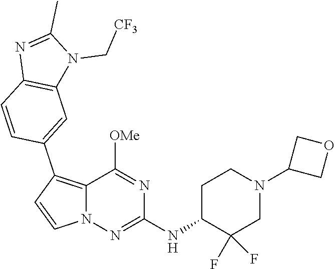 [SMILES: COc1nc(N[C@@H]2CCN(C3COC3)CC2(F)F)nn2ccc(-c3ccc4nc(C)n(CC(F)(F)F)c4c3)c12] |
| 602 | 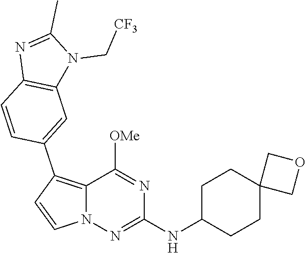 [SMILES: COc1nc(NC2CCC3(CC2)COC3)nn2ccc(-c3ccc4nc(C)n(CC(F)(F)F)c4c3)c12] |
| 603 | 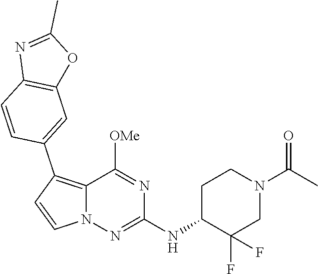 [SMILES: COc1nc(N[C@@H]2CCN(C(C)=O)CC2(F)F)nn2ccc(-c3ccc4nc(C)oc4c3)c12] |
| 604 | 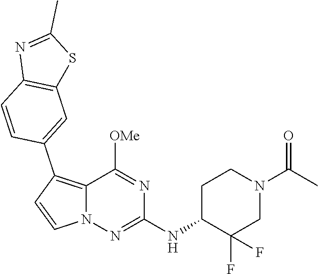 [SMILES: COc1nc(N[C@@H]2CCN(C(C)=O)CC2(F)F)nn2ccc(-c3ccc4nc(C)sc4c3)c12] |
| 605 | 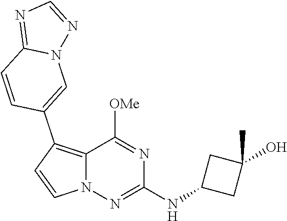 [SMILES: COc1nc(N[C@H]2C[C@@](C)(O)C2)nn2ccc(-c3ccc4ncnn4c3)c12] |
| 606 | 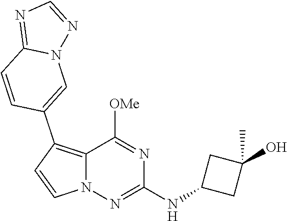 [SMILES: COc1nc(N[C@H]2C[C@](C)(O)C2)nn2ccc(-c3ccc4ncnn4c3)c12] |
| 607 | 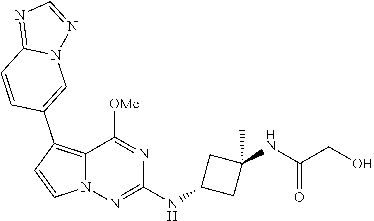 [SMILES: COc1nc(N[C@H]2C[C@](C)(NC(=O)CO)C2)nn2ccc(-c3ccc4ncnn4c3)c12] |
| 608 | 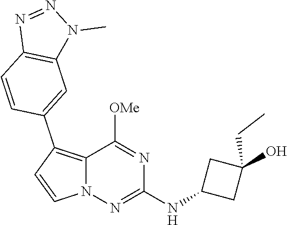 [SMILES: CC[C@]1(O)C[C@H](Nc2nc(OC)c3c(-c4ccc5nnn(C)c5c4)ccn3n2)C1] |
| 609 | 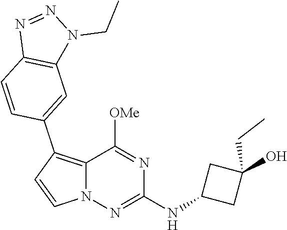 [SMILES: CCn1nnc2ccc(-c3ccn4nc(N[C@H]5C[C@@](O)(CC)C5)nc(OC)c34)cc21] |
| 610 | 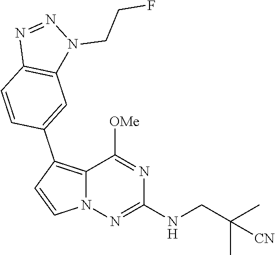 [SMILES: COc1nc(NCC(C)(C)C#N)nn2ccc(-c3ccc4nnn(CCF)c4c3)c12] |
| 611 | 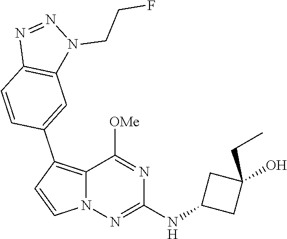 [SMILES: CC[C@]1(O)C[C@@H](Nc2nc(OC)c3c(-c4ccc5nnn(CCF)c5c4)ccn3n2)C1] |
| 612 | 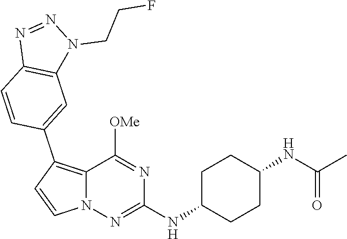 [SMILES: COc1nc(N[C@H]2CC[C@@H](NC(C)=O)CC2)nn2ccc(-c3ccc4nnn(CCF)c4c3)c12] |
| 613 | 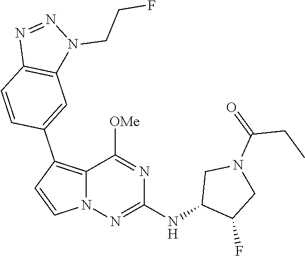 [SMILES: CCC(=O)N1C[C@H](F)[C@H](Nc2nc(OC)c3c(-c4ccc5nnn(CCF)c5c4)ccn3n2)C1] |
| 614 | 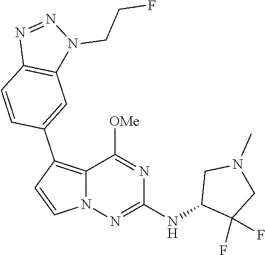 [SMILES: COc1nc(N[C@@H]2CN(C)CC2(F)F)nn2ccc(-c3ccc4nnn(CCF)c4c3)c12] |
| 615 | 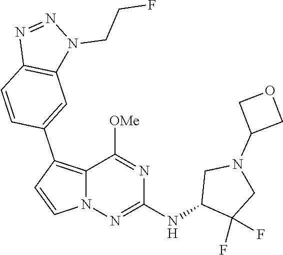 [SMILES: COc1nc(N[C@@H]2CN(C3COC3)CC2(F)F)nn2ccc(-c3ccc4nnn(CCF)c4c3)c12] |
| 616 | 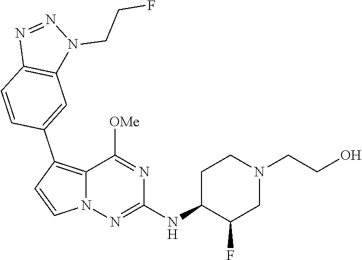 [SMILES: COc1nc(N[C@H]2CCN(CCO)C[C@H]2F)nn2ccc(-c3ccc4nnn(CCF)c4c3)c12] |
| 617 | 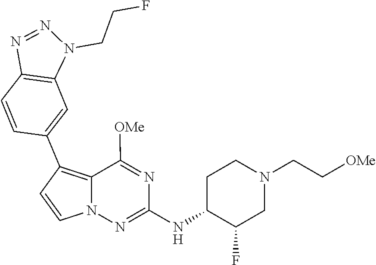 [SMILES: COCCN1CC[C@@H](Nc2nc(OC)c3c(-c4ccc5nnn(CCF)c5c4)ccn3n2)[C@@H](F)C1] |
| 618 | 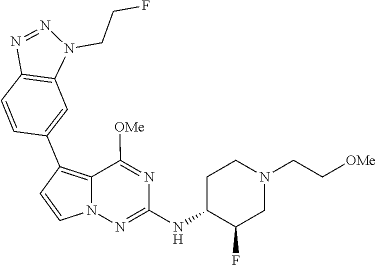 [SMILES: COCCN1CC[C@@H](Nc2nc(OC)c3c(-c4ccc5nnn(CCF)c5c4)ccn3n2)[C@H](F)C1] |
| 619 | 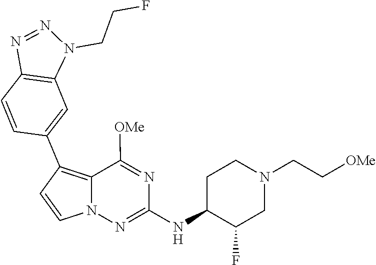 [SMILES: COCCN1CC[C@H](Nc2nc(OC)c3c(-c4ccc5nnn(CCF)c5c4)ccn3n2)[C@@H](F)C1] |
| 620 | 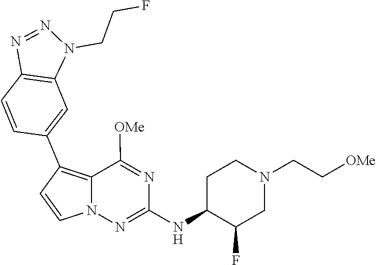 [SMILES: COCCN1CC[C@H](Nc2nc(OC)c3c(-c4ccc5nnn(CCF)c5c4)ccn3n2)[C@H](F)C1] |
| 621 | 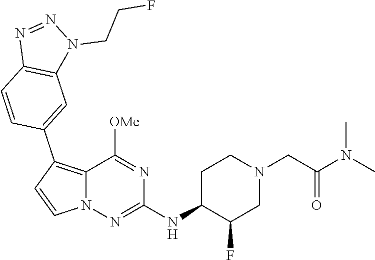 [SMILES: COc1nc(N[C@H]2CCN(CC(=O)N(C)C)C[C@H]2F)nn2ccc(-c3ccc4nnn(CCF)c4c3)c12] |
| 622 | 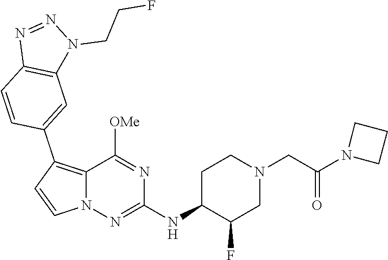 [SMILES: COc1nc(N[C@H]2CCN(CC(=O)N3CCC3)C[C@H]2F)nn2ccc(-c3ccc4nnn(CCF)c4c3)c12] |
| 623 | 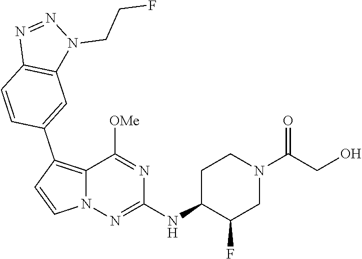 [SMILES: COc1nc(N[C@H]2CCN(C(=O)CO)C[C@H]2F)nn2ccc(-c3ccc4nnn(CCF)c4c3)c12] |
| 624 | 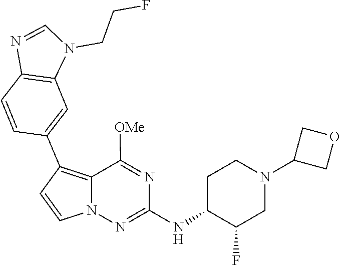 [SMILES: COc1nc(N[C@@H]2CCN(C3COC3)C[C@@H]2F)nn2ccc(-c3ccc4ncn(CCF)c4c3)c12] |
| 625 | 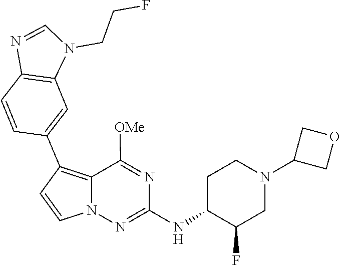 [SMILES: COc1nc(N[C@@H]2CCN(C3COC3)C[C@H]2F)nn2ccc(-c3ccc4ncn(CCF)c4c3)c12] |
| 626 | 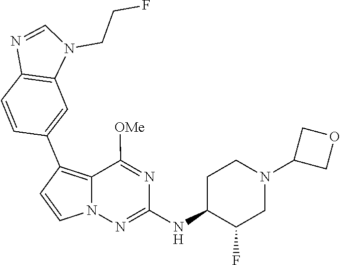 [SMILES: COc1nc(N[C@H]2CCN(C3COC3)C[C@@H]2F)nn2ccc(-c3ccc4ncn(CCF)c4c3)c12] |
| 627 | 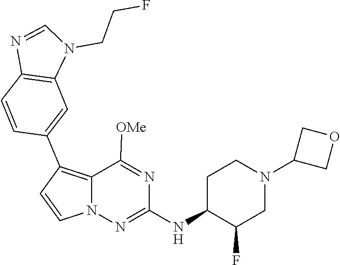 [SMILES: COc1nc(N[C@H]2CCN(C3COC3)C[C@H]2F)nn2ccc(-c3ccc4ncn(CCF)c4c3)c12] |
| 628 | 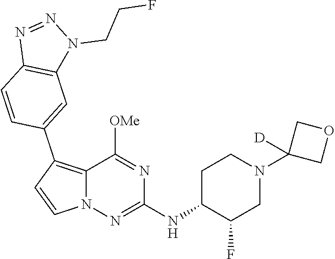 [SMILES: [2H]C1(N2CC[C@@H](Nc3nc(OC)c4c(-c5ccc6nnn(CCF)c6c5)ccn4n3)[C@@H](F)C2)COC1] |
| 629 | 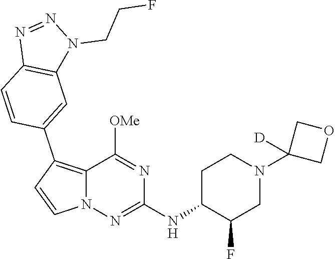 [SMILES: [2H]C1(N2CC[C@@H](Nc3nc(OC)c4c(-c5ccc6nnn(CCF)c6c5)ccn4n3)[C@H](F)C2)COC1] |
| 630 | 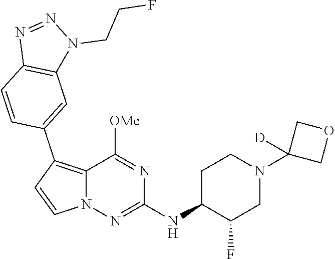 [SMILES: [2H]C1(N2CC[C@H](Nc3nc(OC)c4c(-c5ccc6nnn(CCF)c6c5)ccn4n3)[C@@H](F)C2)COC1] |
| 631 | 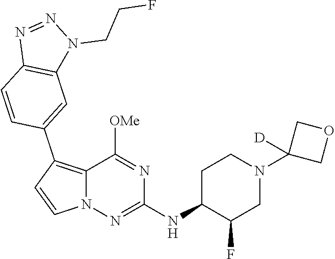 [SMILES: [2H]C1(N2CC[C@H](Nc3nc(OC)c4c(-c5ccc6nnn(CCF)c6c5)ccn4n3)[C@H](F)C2)COC1] |
| 632 | 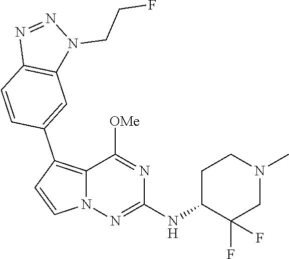 [SMILES: COc1nc(N[C@@H]2CCN(C)CC2(F)F)nn2ccc(-c3ccc4nnn(CCF)c4c3)c12] |
| 633 | 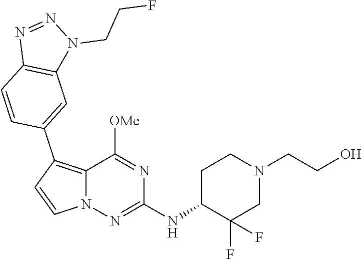 |
| 634 | 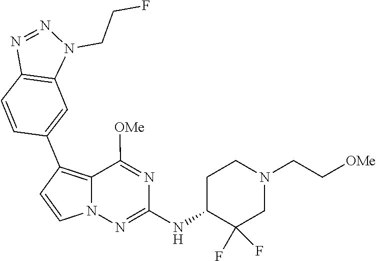 [SMILES: COCCN1CC[C@@H](Nc2nc(OC)c3c(-c4ccc5nnn(CCF)c5c4)ccn3n2)C(F)(F)C1] |
| 635 | 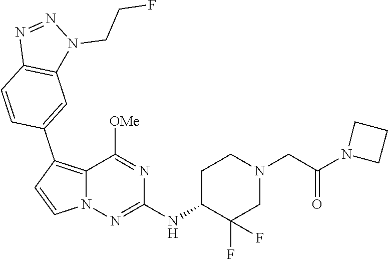 [SMILES: COc1nc(N[C@@H]2CCN(CC(=O)N3CCC3)CC2(F)F)nn2ccc(-c3ccc4nnn(CCF)c4c3)c12] |
| 636 | 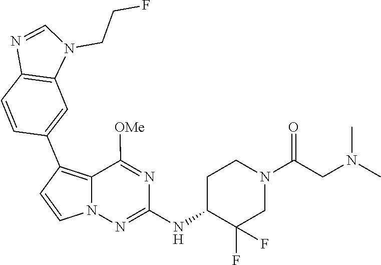 [SMILES: COc1nc(N[C@@H]2CCN(C(=O)CN(C)C)CC2(F)F)nn2ccc(-c3ccc4ncn(CCF)c4c3)c12] |
| 637 | 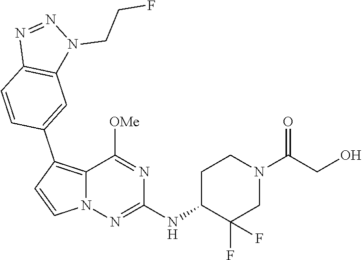 [SMILES: COc1nc(N[C@@H]2CCN(C(=O)CO)CC2(F)F)nn2ccc(-c3ccc4nnn(CCF)c4c3)c12] |
| 638 | 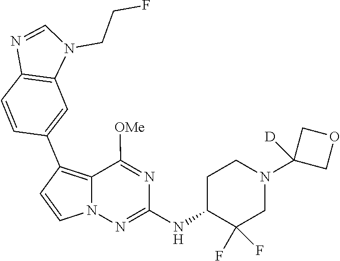 [SMILES: [2H]C1(N2CC[C@@H](Nc3nc(OC)c4c(-c5ccc6ncn(CCF)c6c5)ccn4n3)C(F)(F)C2)COC1] |
| 639 | 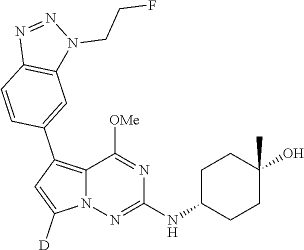 [SMILES: [2H]c1cc(-c2ccc3nnn(CCF)c3c2)c2c(OC)nc(N[C@H]3CC[C@@](C)(O)CC3)nn12] |
| 640 | 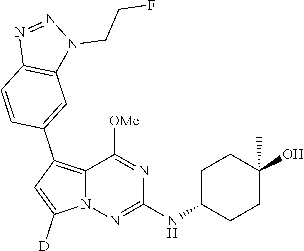 [SMILES: [2H]c1cc(-c2ccc3nnn(CCF)c3c2)c2c(OC)nc(N[C@H]3CC[C@](C)(O)CC3)nn12] |
| 641 | 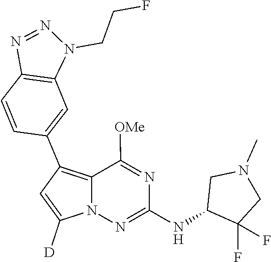 [SMILES: [2H]c1cc(-c2ccc3nnn(CCF)c3c2)c2c(OC)nc(N[C@@H]3CN(C)CC3(F)F)nn12] |
| 642 | 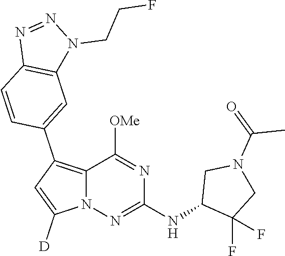 [SMILES: [2H]c1cc(-c2ccc3nnn(CCF)c3c2)c2c(OC)nc(N[C@@H]3CN(C(C)=O)CC3(F)F)nn12] |
| 643 | 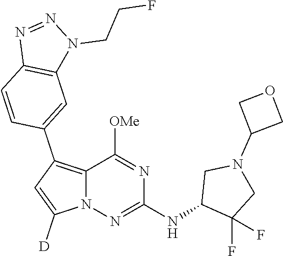 [SMILES: [2H]c1cc(-c2ccc3nnn(CCF)c3c2)c2c(OC)nc(N[C@@H]3CN(C4COC4)CC3(F)F)nn12] |
| 644 | 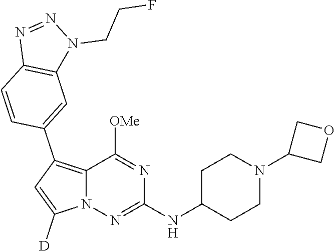 [SMILES: [2H]c1cc(-c2ccc3nnn(CCF)c3c2)c2c(OC)nc(NC3CCN(C4COC4)CC3)nn12] |
| 645 | 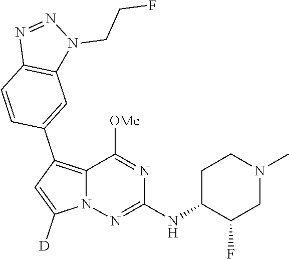 [SMILES: [2H]c1cc(-c2ccc3nnn(CCF)c3c2)c2c(OC)nc(N[C@@H]3CCN(C)C[C@@H]3F)nn12] |
| 646 | 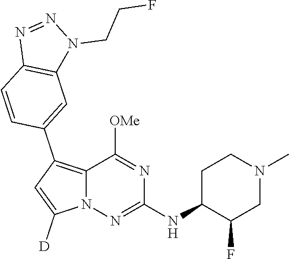 [SMILES: [2H]c1cc(-c2ccc3nnn(CCF)c3c2)c2c(OC)nc(N[C@H]3CCN(C)C[C@H]3F)nn12] |
| 647 | 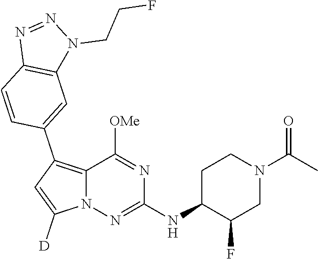 [SMILES: [2H]c1cc(-c2ccc3nnn(CCF)c3c2)c2c(OC)nc(N[C@H]3CCN(C(C)=O)C[C@H]3F)nn12] |
| 648 | 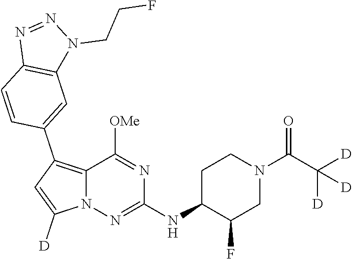 [SMILES: [2H]c1cc(-c2ccc3nnn(CCF)c3c2)c2c(OC)nc(N[C@H]3CCN(C(=O)C([2H])([2H])[2H])C[C@H]3F)nn12] |
| 649 | 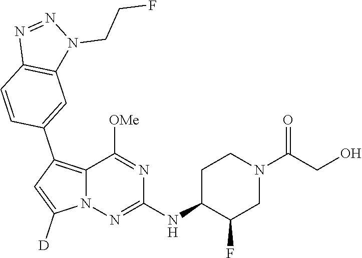 [SMILES: [2H]c1cc(-c2ccc3nnn(CCF)c3c2)c2c(OC)nc(N[C@H]3CCN(C(=O)CO)C[C@H]3F)nn12] |
| 650 | 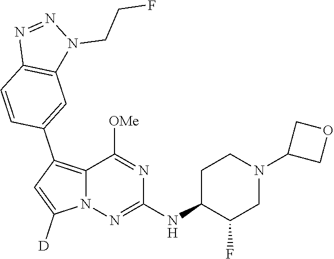 [SMILES: [2H]c1cc(-c2ccc3nnn(CCF)c3c2)c2c(OC)nc(N[C@H]3CCN(C4COC4)C[C@@H]3F)nn12] |
| 651 | 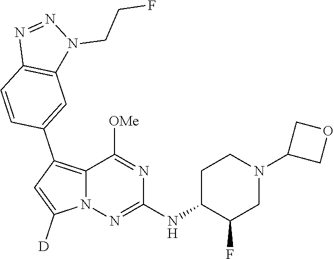 [SMILES: [2H]c1cc(-c2ccc3nnn(CCF)c3c2)c2c(OC)nc(N[C@@H]3CCN(C4COC4)C[C@H]3F)nn12] |
| 652 | 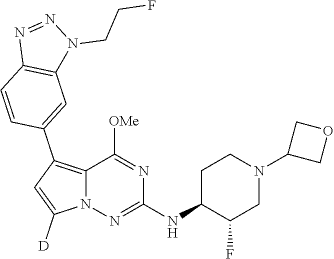 [SMILES: [2H]c1cc(-c2ccc3nnn(CCF)c3c2)c2c(OC)nc(N[C@H]3CCN(C4COC4)C[C@@H]3F)nn12] |
| 653 | 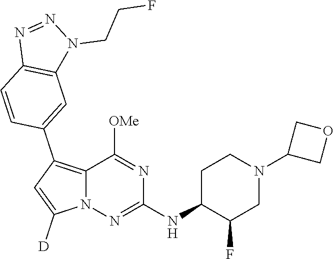 [SMILES: [2H]c1cc(-c2ccc3nnn(CCF)c3c2)c2c(OC)nc(N[C@H]3CCN(C4COC4)C[C@H]3F)nn12] |
| 654 | 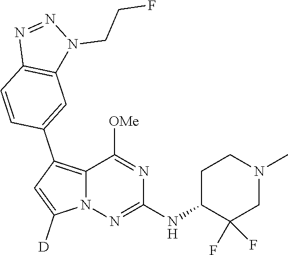 [SMILES: [2H]c1cc(-c2ccc3nnn(CCF)c3c2)c2c(OC)nc(N[C@@H]3CCN(C)CC3(F)F)nn12] |
| 655 | 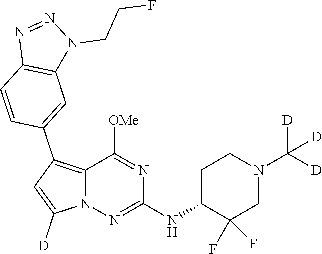 [SMILES: [2H]c1cc(-c2ccc3nnn(CCF)c3c2)c2c(OC)nc(N[C@@H]3CCN(C([2H])([2H])[2H])CC3(F)F)nn12] |
| 656 | 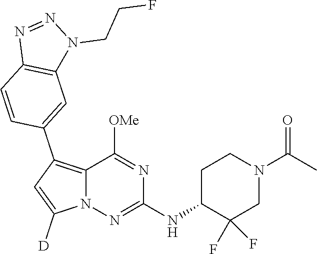 [SMILES: [2H]c1cc(-c2ccc3nnn(CCF)c3c2)c2c(OC)nc(N[C@@H]3CCN(C(C)=O)CC3(F)F)nn12] |
| 657 | 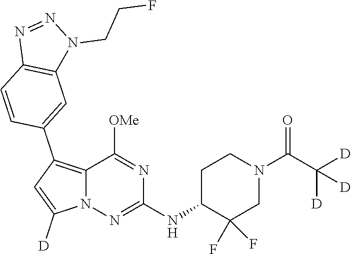 [SMILES: [2H]c1cc(-c2ccc3nnn(CCF)c3c2)c2c(OC)nc(N[C@@H]3CCN(C(=O)C([2H])([2H])[2H])CC3(F)F)nn12] |
| 658 | 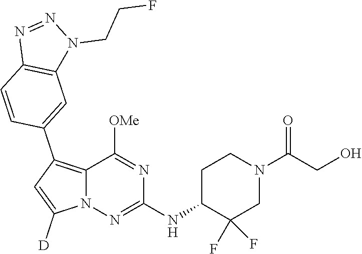 [SMILES: [2H]c1cc(-c2ccc3nnn(CCF)c3c2)c2c(OC)nc(N[C@@H]3CCN(C(=O)CO)CC3(F)F)nn12] |
| 659 | 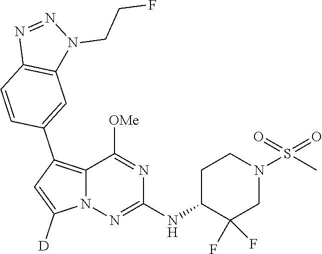 [SMILES: [2H]c1cc(-c2ccc3nnn(CCF)c3c2)c2c(OC)nc(N[C@@H]3CCN(S(C)(=O)=O)CC3(F)F)nn12] |
| 660 | 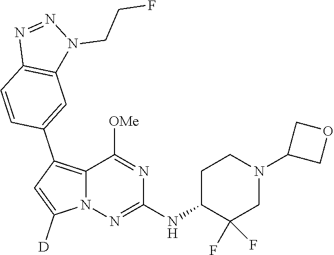 [SMILES: [2H]c1cc(-c2ccc3nnn(CCF)c3c2)c2c(OC)nc(N[C@@H]3CCN(C4COC4)CC3(F)F)nn12] |
| 661 | 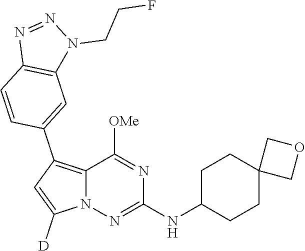 [SMILES: [2H]c1cc(-c2ccc3nnn(CCF)c3c2)c2c(OC)nc(NC3CCC4(CC3)COC4)nn12] |
| 662 | 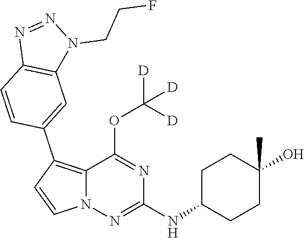 [SMILES: [2H]C([2H])([2H])Oc1nc(N[C@H]2CC[C@@](C)(O)CC2)nn2ccc(-c3ccc4nnn(CCF)c4c3)c12] |
| 663 | 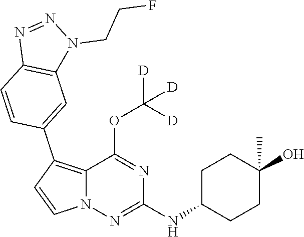 [SMILES: [2H]C([2H])([2H])Oc1nc(N[C@H]2CC[C@](C)(O)CC2)nn2ccc(-c3ccc4nnn(CCF)c4c3)c12] |
| 664 | 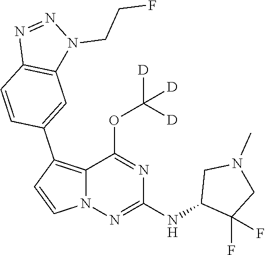 [SMILES: [2H]C([2H])([2H])Oc1nc(N[C@@H]2CN(C)CC2(F)F)nn2ccc(-c3ccc4nnn(CCF)c4c3)c12] |
| 665 | 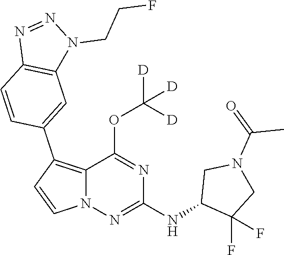 [SMILES: [2H]C([2H])([2H])Oc1nc(N[C@@H]2CN(C(C)=O)CC2(F)F)nn2ccc(-c3ccc4nnn(CCF)c4c3)c12] |
| 666 | 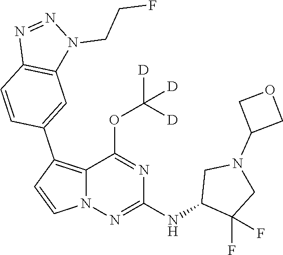 [SMILES: [2H]C([2H])([2H])Oc1nc(N[C@@H]2CN(C3COC3)CC2(F)F)nn2ccc(-c3ccc4nnn(CCF)c4c3)c12] |
| 667 | 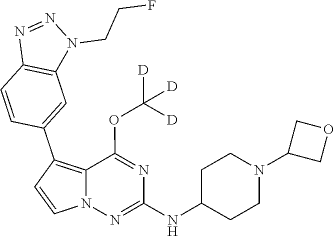 [SMILES: [2H]C([2H])([2H])Oc1nc(NC2CCN(C3COC3)CC2)nn2ccc(-c3ccc4nnn(CCF)c4c3)c12] |
| 668 | 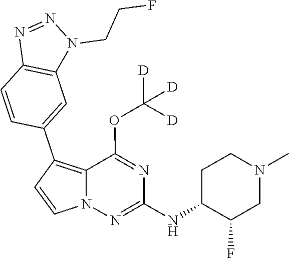 [SMILES: [2H]C([2H])([2H])Oc1nc(N[C@@H]2CCN(C)C[C@@H]2F)nn2ccc(-c3ccc4nnn(CCF)c4c3)c12] |
| 669 | 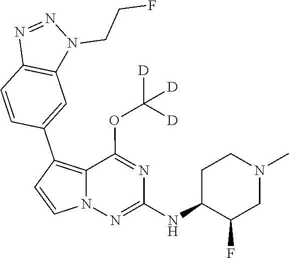 [SMILES: [2H]C([2H])([2H])Oc1nc(N[C@H]2CCN(C)C[C@H]2F)nn2ccc(-c3ccc4nnn(CCF)c4c3)c12] |
| 670 | 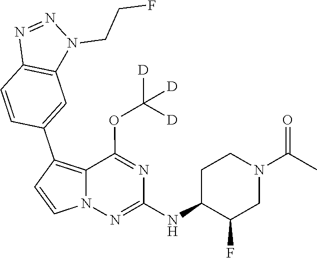 [SMILES: [2H]C([2H])([2H])Oc1nc(N[C@H]2CCN(C(C)=O)C[C@H]2F)nn2ccc(-c3ccc4nnn(CCF)c4c3)c12] |
| 671 | 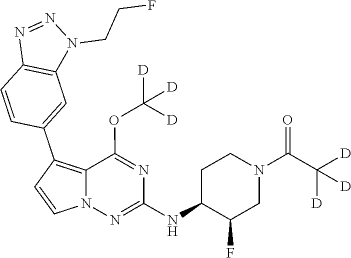 [SMILES: [2H]C([2H])([2H])Oc1nc(N[C@H]2CCN(C(=O)C([2H])([2H])[2H])C[C@H]2F)nn2ccc(-c3ccc4nnn(CCF)c4c3)c12] |
| 672 | 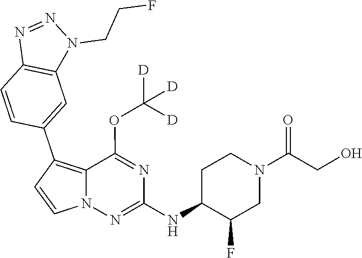 [SMILES: [2H]C([2H])([2H])Oc1nc(N[C@H]2CCN(C(=O)CO)C[C@H]2F)nn2ccc(-c3ccc4nnn(CCF)c4c3)c12] |
| 673 | 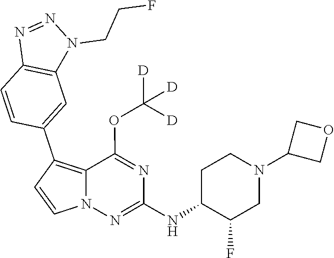 [SMILES: [2H]C([2H])([2H])Oc1nc(N[C@@H]2CCN(C3COC3)C[C@@H]2F)nn2ccc(-c3ccc4nnn(CCF)c4c3)c12] |
| 674 | 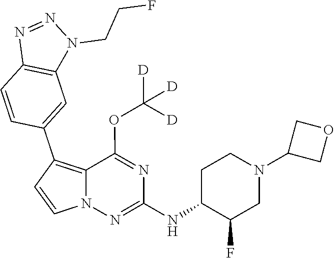 [SMILES: [2H]C([2H])([2H])Oc1nc(N[C@@H]2CCN(C3COC3)C[C@H]2F)nn2ccc(-c3ccc4nnn(CCF)c4c3)c12] |
| 675 | 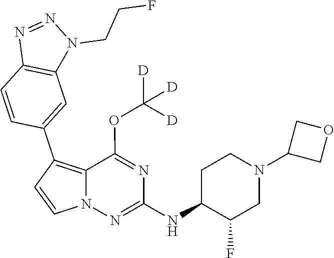 [SMILES: [2H]C([2H])([2H])Oc1nc(N[C@H]2CCN(C3COC3)C[C@@H]2F)nn2ccc(-c3ccc4nnn(CCF)c4c3)c12] |
| 676 | 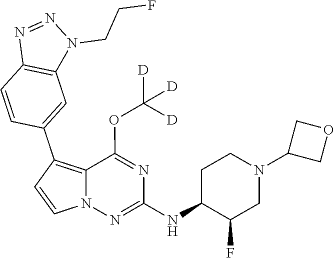 [SMILES: [2H]C([2H])([2H])Oc1nc(N[C@H]2CCN(C3COC3)C[C@H]2F)nn2ccc(-c3ccc4nnn(CCF)c4c3)c12] |
| 677 | 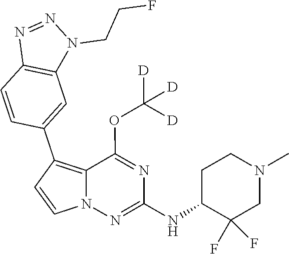 [SMILES: [2H]C([2H])([2H])Oc1nc(N[C@@H]2CCN(C)CC2(F)F)nn2ccc(-c3ccc4nnn(CCF)c4c3)c12] |
| 678 | 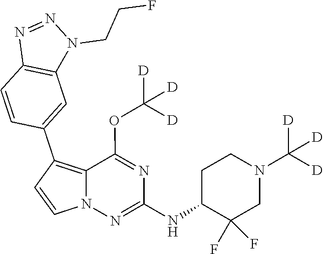 [SMILES: [2H]C([2H])([2H])Oc1nc(N[C@@H]2CCN(C([2H])([2H])[2H])CC2(F)F)nn2ccc(-c3ccc4nnn(CCF)c4c3)c12] |
| 679 | 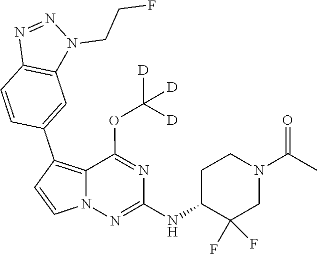 [SMILES: [2H]C([2H])([2H])Oc1nc(N[C@@H]2CCN(C(C)=O)CC2(F)F)nn2ccc(-c3ccc4nnn(CCF)c4c3)c12] |
| 680 | 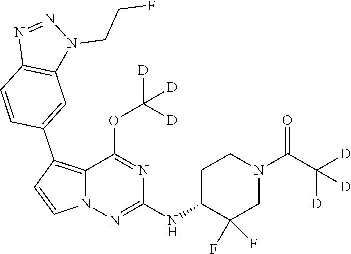 [SMILES: [2H]C([2H])([2H])Oc1nc(N[C@@H]2CCN(C(=O)C([2H])([2H])[2H])CC2(F)F)nn2ccc(-c3ccc4nnn(CCF)c4c3)c12] |
| 681 | 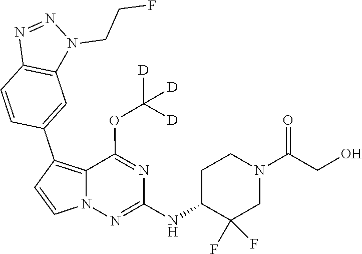 [SMILES: [2H]C([2H])([2H])Oc1nc(N[C@@H]2CCN(C(=O)CO)CC2(F)F)nn2ccc(-c3ccc4nnn(CCF)c4c3)c12] |
| 682 | 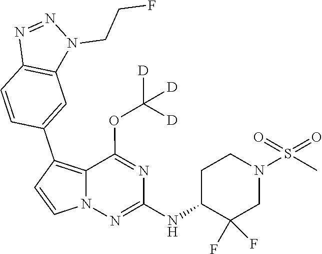 [SMILES: [2H]C([2H])([2H])Oc1nc(N[C@@H]2CCN(S(C)(=O)=O)CC2(F)F)nn2ccc(-c3ccc4nnn(CCF)c4c3)c12] |
| 683 | 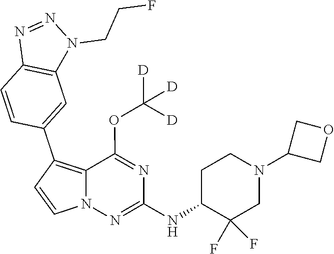 [SMILES: [2H]C([2H])([2H])Oc1nc(N[C@@H]2CCN(C3COC3)CC2(F)F)nn2ccc(-c3ccc4nnn(CCF)c4c3)c12] |
| 684 | 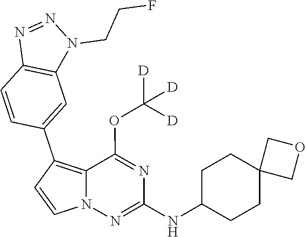 [SMILES: [2H]C([2H])([2H])Oc1nc(NC2CCC3(CC2)COC3)nn2ccc(-c3ccc4nnn(CCF)c4c3)c12] |
| 685 | 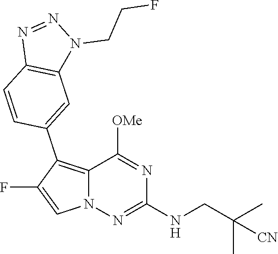 [SMILES: COc1nc(NCC(C)(C)C#N)nn2cc(F)c(-c3ccc4nnn(CCF)c4c3)c12] |
| 686 | 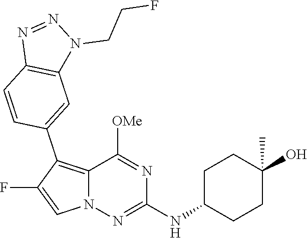 [SMILES: COc1nc(N[C@H]2CC[C@](C)(O)CC2)nn2cc(F)c(-c3ccc4nnn(CCF)c4c3)c12] |
| 687 | 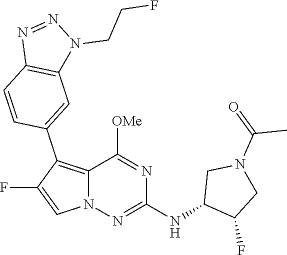 [SMILES: COc1nc(N[C@@H]2CN(C(C)=O)C[C@@H]2F)nn2cc(F)c(-c3ccc4nnn(CCF)c4c3)c12] |
| 688 | 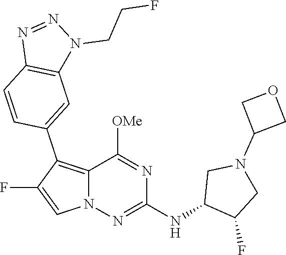 [SMILES: COc1nc(N[C@@H]2CN(C3COC3)C[C@@H]2F)nn2cc(F)c(-c3ccc4nnn(CCF)c4c3)c12] |
| 689 | 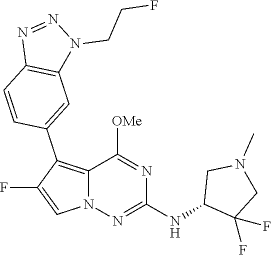 [SMILES: COc1nc(N[C@@H]2CN(C)CC2(F)F)nn2cc(F)c(-c3ccc4nnn(CCF)c4c3)c12] |
| 690 | 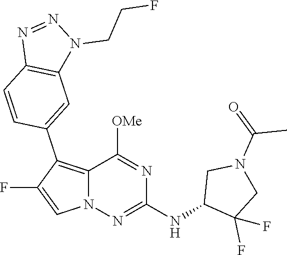 [SMILES: COc1nc(N[C@@H]2CN(C(C)=O)CC2(F)F)nn2cc(F)c(-c3ccc4nnn(CCF)c4c3)c12] |
| 691 | 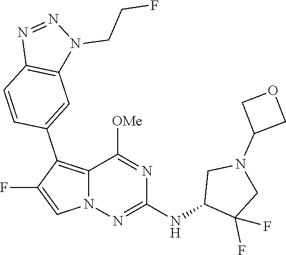 [SMILES: COc1nc(N[C@@H]2CN(C3COC3)CC2(F)F)nn2cc(F)c(-c3ccc4nnn(CCF)c4c3)c12] |
| 692 | 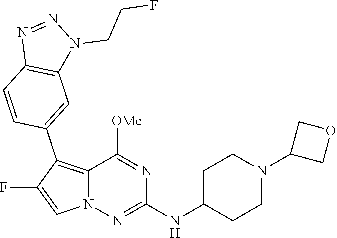 [SMILES: COc1nc(NC2CCN(C3COC3)CC2)nn2cc(F)c(-c3ccc4nnn(CCF)c4c3)c12] |
| 693 | 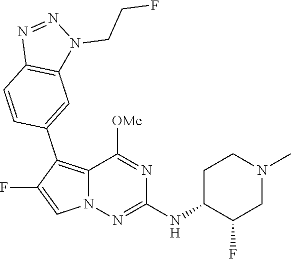 [SMILES: COc1nc(N[C@@H]2CCN(C)C[C@@H]2F)nn2cc(F)c(-c3ccc4nnn(CCF)c4c3)c12] |
| 694 | 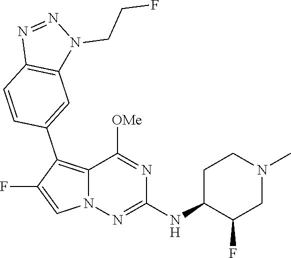 [SMILES: COc1nc(N[C@H]2CCN(C)C[C@H]2F)nn2cc(F)c(-c3ccc4nnn(CCF)c4c3)c12] |
| 695 | 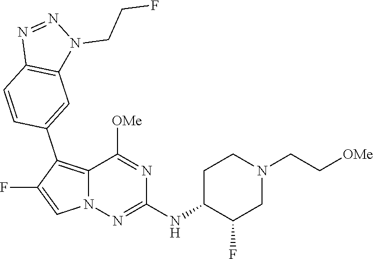 [SMILES: COCCN1CC[C@@H](Nc2nc(OC)c3c(-c4ccc5nnn(CCF)c5c4)c(F)cn3n2)[C@@H](F)C1] |
| 696 | 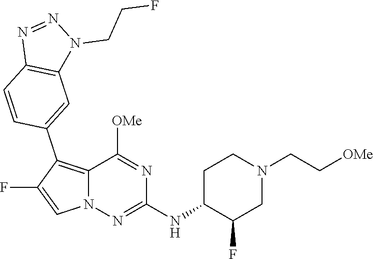 [SMILES: COCCN1CC[C@@H](Nc2nc(OC)c3c(-c4ccc5nnn(CCF)c5c4)c(F)cn3n2)[C@H](F)C1] |
| 697 | 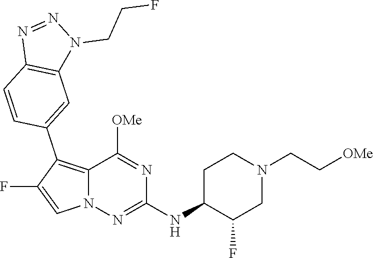 [SMILES: COCCN1CC[C@H](Nc2nc(OC)c3c(-c4ccc5nnn(CCF)c5c4)c(F)cn3n2)[C@@H](F)C1] |
| 698 | 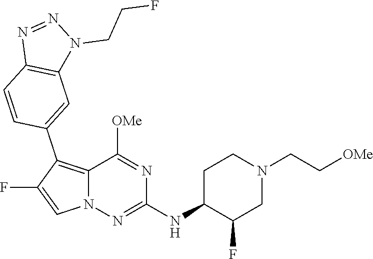 [SMILES: COCCN1CC[C@H](Nc2nc(OC)c3c(-c4ccc5nnn(CCF)c5c4)c(F)cn3n2)[C@H](F)C1] |
| 699 | 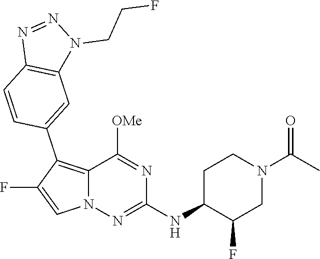 [SMILES: COc1nc(N[C@H]2CCN(C(C)=O)C[C@H]2F)nn2cc(F)c(-c3ccc4nnn(CCF)c4c3)c12] |
| 700 | 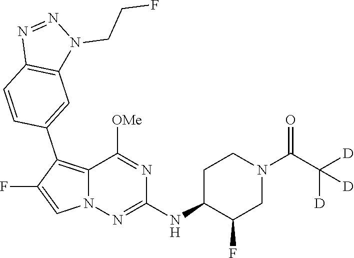 [SMILES: [2H]C([2H])([2H])C(=O)N1CC[C@H](Nc2nc(OC)c3c(-c4ccc5nnn(CCF)c5c4)c(F)cn3n2)[C@H](F)C1] |
| 701 | 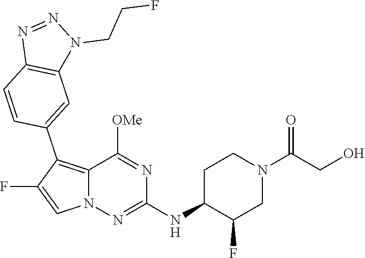 [SMILES: COc1nc(N[C@H]2CCN(C(=O)CO)C[C@H]2F)nn2cc(F)c(-c3ccc4nnn(CCF)c4c3)c12] |
| 702 | 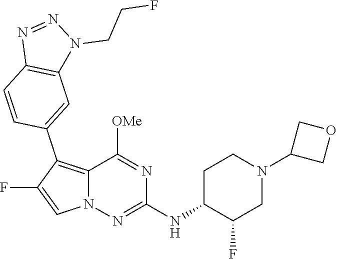 [SMILES: COc1nc(N[C@@H]2CCN(C3COC3)C[C@@H]2F)nn2cc(F)c(-c3ccc4nnn(CCF)c4c3)c12] |
| 703 | 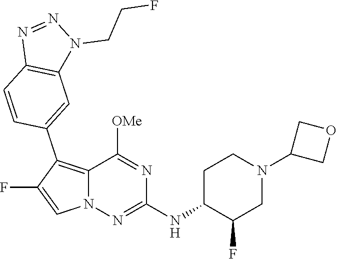 [SMILES: COc1nc(N[C@@H]2CCN(C3COC3)C[C@H]2F)nn2cc(F)c(-c3ccc4nnn(CCF)c4c3)c12] |
| 704 | 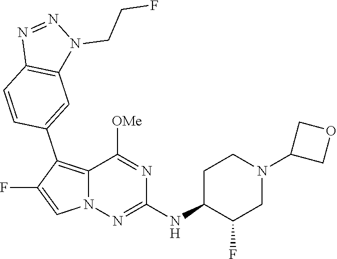 [SMILES: COc1nc(N[C@H]2CCN(C3COC3)C[C@@H]2F)nn2cc(F)c(-c3ccc4nnn(CCF)c4c3)c12] |
| 705 | 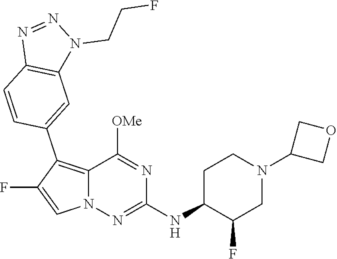 [SMILES: COc1nc(N[C@H]2CCN(C3COC3)C[C@H]2F)nn2cc(F)c(-c3ccc4nnn(CCF)c4c3)c12] |
| 706 | 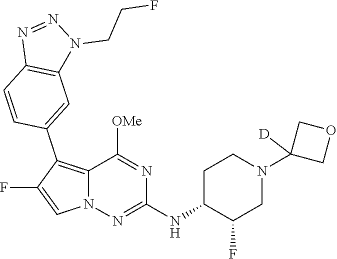 [SMILES: [2H]C1(N2CC[C@@H](Nc3nc(OC)c4c(-c5ccc6nnn(CCF)c6c5)c(F)cn4n3)[C@@H](F)C2)COC1] |
| 707 | 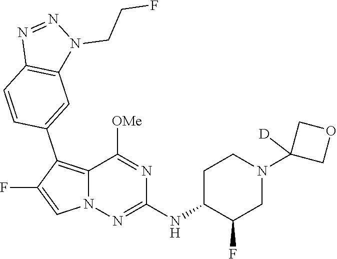 [SMILES: [2H]C1(N2CC[C@@H](Nc3nc(OC)c4c(-c5ccc6nnn(CCF)c6c5)c(F)cn4n3)[C@H](F)C2)COC1] |
| 708 | 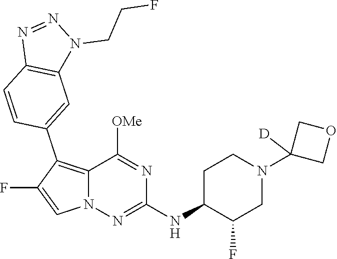 [SMILES: [2H]C1(N2CC[C@H](Nc3nc(OC)c4c(-c5ccc6nnn(CCF)c6c5)c(F)cn4n3)[C@@H](F)C2)COC1] |
| 709 | 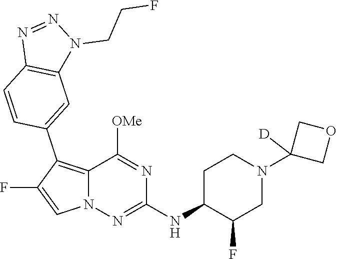 [SMILES: [2H]C1(N2CC[C@H](Nc3nc(OC)c4c(-c5ccc6nnn(CCF)c6c5)c(F)cn4n3)[C@H](F)C2)COC1] |
| 710 | 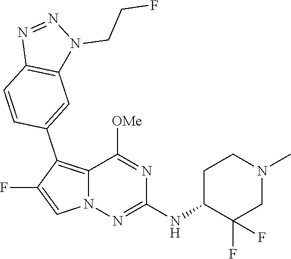 [SMILES: COc1nc(N[C@@H]2CCN(C)CC2(F)F)nn2cc(F)c(-c3ccc4nnn(CCF)c4c3)c12] |
| 711 | 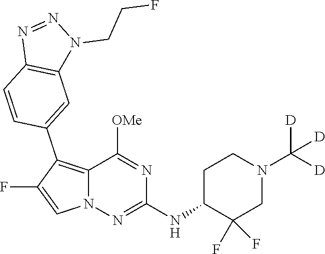 [SMILES: [2H]C([2H])([2H])N1CC[C@@H](Nc2nc(OC)c3c(-c4ccc5nnn(CCF)c5c4)c(F)cn3n2)C(F)(F)C1] |
| 712 | 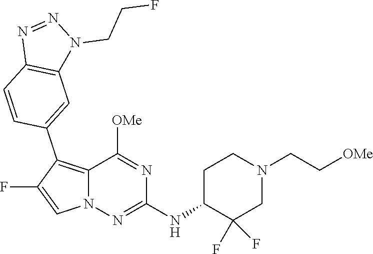 [SMILES: COCCN1CC[C@@H](Nc2nc(OC)c3c(-c4ccc5nnn(CCF)c5c4)c(F)cn3n2)C(F)(F)C1] |
| 713 | 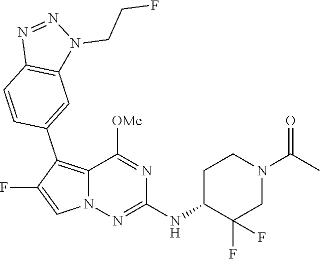 [SMILES: COc1nc(N[C@@H]2CCN(C(C)=O)CC2(F)F)nn2cc(F)c(-c3ccc4nnn(CCF)c4c3)c12] |
| 714 | 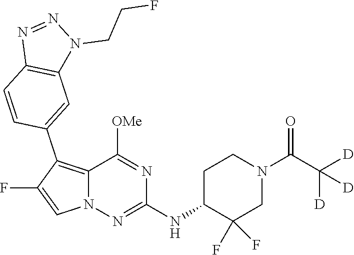 [SMILES: [2H]C([2H])([2H])C(=O)N1CC[C@@H](Nc2nc(OC)c3c(-c4ccc5nnn(CCF)c5c4)c(F)cn3n2)C(F)(F)C1] |
| 715 | 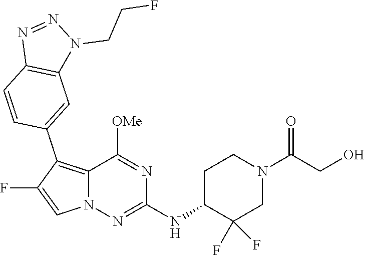 [SMILES: COc1nc(N[C@@H]2CCN(C(=O)CO)CC2(F)F)nn2cc(F)c(-c3ccc4nnn(CCF)c4c3)c12] |
| 716 | 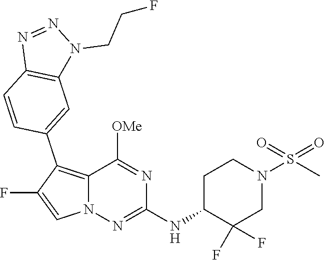 [SMILES: COc1nc(N[C@@H]2CCN(S(C)(=O)=O)CC2(F)F)nn2cc(F)c(-c3ccc4nnn(CCF)c4c3)c12] |
| 717 | 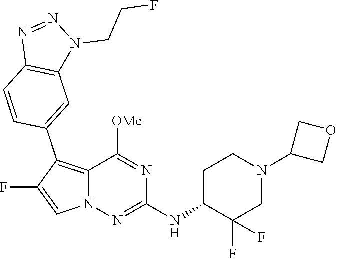 [SMILES: COc1nc(N[C@@H]2CCN(C3COC3)CC2(F)F)nn2cc(F)c(-c3ccc4nnn(CCF)c4c3)c12] |
| 718 | 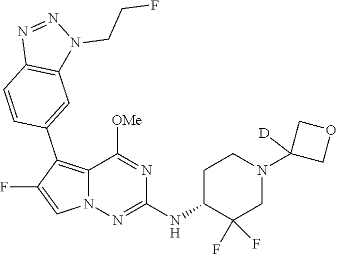 [SMILES: [2H]C1(N2CC[C@@H](Nc3nc(OC)c4c(-c5ccc6nnn(CCF)c6c5)c(F)cn4n3)C(F)(F)C2)COC1] |
| 719 | 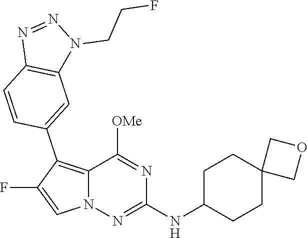 [SMILES: COc1nc(NC2CCC3(CC2)COC3)nn2cc(F)c(-c3ccc4nnn(CCF)c4c3)c12] |
| 720 | 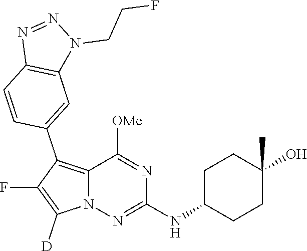 [SMILES: [2H]c1c(F)c(-c2ccc3nnn(CCF)c3c2)c2c(OC)nc(N[C@H]3CC[C@@](C)(O)CC3)nn12] |
| 721 | 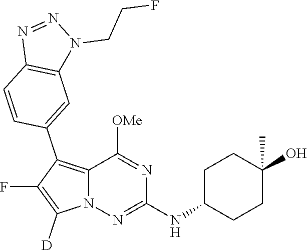 [SMILES: [2H]c1c(F)c(-c2ccc3nnn(CCF)c3c2)c2c(OC)nc(N[C@H]3CC[C@](C)(O)CC3)nn12] |
| 722 | 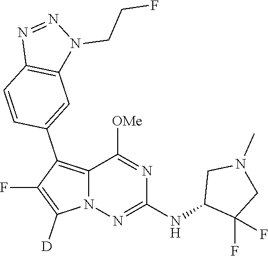 [SMILES: [2H]c1c(F)c(-c2ccc3nnn(CCF)c3c2)c2c(OC)nc(N[C@@H]3CN(C)CC3(F)F)nn12] |
| 723 | 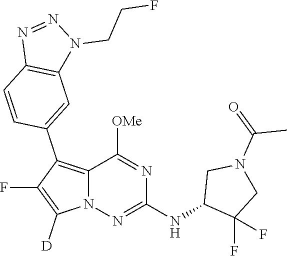 [SMILES: [2H]c1c(F)c(-c2ccc3nnn(CCF)c3c2)c2c(OC)nc(N[C@@H]3CN(C(C)=O)CC3(F)F)nn12] |
| 724 | 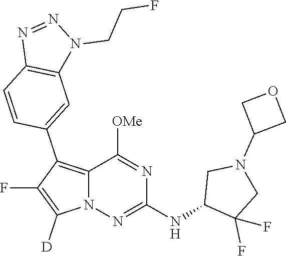 [SMILES: [2H]c1c(F)c(-c2ccc3nnn(CCF)c3c2)c2c(OC)nc(N[C@@H]3CN(C4COC4)CC3(F)F)nn12] |
| 725 | 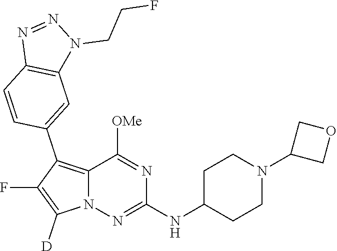 [SMILES: [2H]c1c(F)c(-c2ccc3nnn(CCF)c3c2)c2c(OC)nc(NC3CCN(C4COC4)CC3)nn12] |
| 726 | 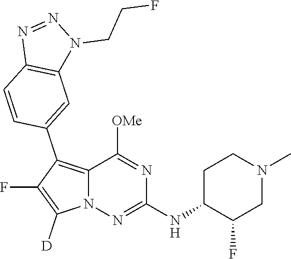 [SMILES: [2H]c1c(F)c(-c2ccc3nnn(CCF)c3c2)c2c(OC)nc(N[C@@H]3CCN(C)C[C@@H]3F)nn12] |
| 727 | 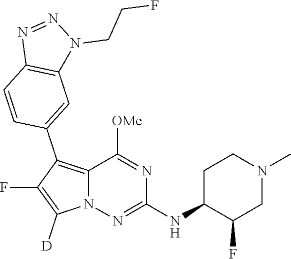 [SMILES: [2H]c1c(F)c(-c2ccc3nnn(CCF)c3c2)c2c(OC)nc(N[C@H]3CCN(C)C[C@H]3F)nn12] |
| 728 | 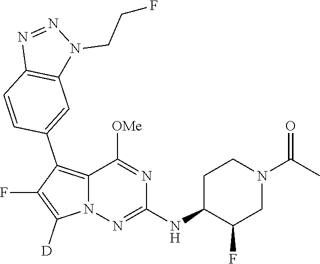 [SMILES: [2H]c1c(F)c(-c2ccc3nnn(CCF)c3c2)c2c(OC)nc(N[C@H]3CCN(C(C)=O)C[C@H]3F)nn12] |
| 729 | 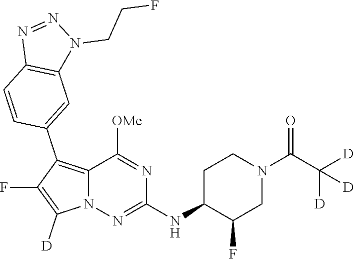 [SMILES: [2H]c1c(F)c(-c2ccc3nnn(CCF)c3c2)c2c(OC)nc(N[C@H]3CCN(C(=O)C([2H])([2H])[2H])C[C@H]3F)nn12] |
| 730 | 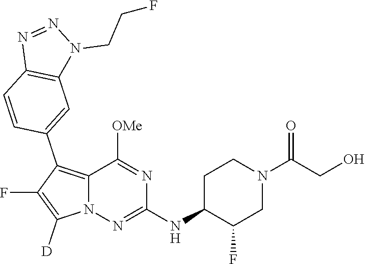 [SMILES: [2H]c1c(F)c(-c2ccc3nnn(CCF)c3c2)c2c(OC)nc(N[C@H]3CCN(C(=O)CO)C[C@@H]3F)nn12] |
| 731 | 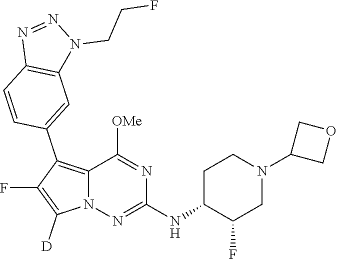 [SMILES: [2H]c1c(F)c(-c2ccc3nnn(CCF)c3c2)c2c(OC)nc(N[C@@H]3CCN(C4COC4)C[C@@H]3F)nn12] |
| 732 | 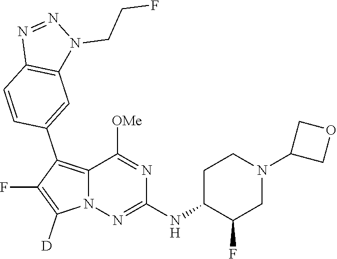 [SMILES: [2H]c1c(F)c(-c2ccc3nnn(CCF)c3c2)c2c(OC)nc(N[C@@H]3CCN(C4COC4)C[C@H]3F)nn12] |
| 733 | 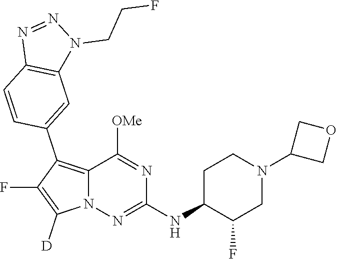 [SMILES: [2H]c1c(F)c(-c2ccc3nnn(CCF)c3c2)c2c(OC)nc(N[C@H]3CCN(C4COC4)C[C@@H]3F)nn12] |
| 734 | 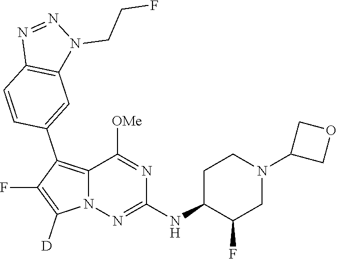 [SMILES: [2H]c1c(F)c(-c2ccc3nnn(CCF)c3c2)c2c(OC)nc(N[C@H]3CCN(C4COC4)C[C@H]3F)nn12] |
| 735 | 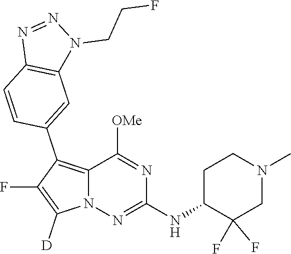 [SMILES: [2H]c1c(F)c(-c2ccc3nnn(CCF)c3c2)c2c(OC)nc(N[C@@H]3CCN(C)CC3(F)F)nn12] |
| 736 | 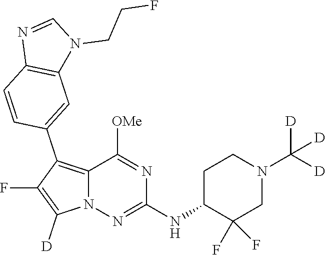 [SMILES: [2H]c1c(F)c(-c2ccc3ncn(CCF)c3c2)c2c(OC)nc(N[C@@H]3CCN(C([2H])([2H])[2H])CC3(F)F)nn12] |
| 737 | 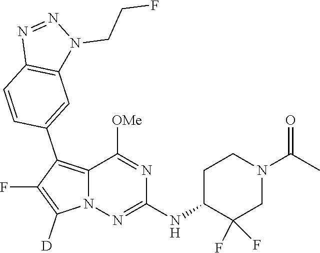 [SMILES: [2H]c1c(F)c(-c2ccc3nnn(CCF)c3c2)c2c(OC)nc(N[C@@H]3CCN(C(C)=O)CC3(F)F)nn12] |
| 738 | 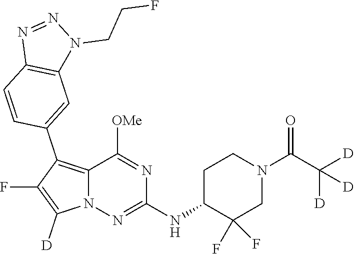 [SMILES: [2H]c1c(F)c(-c2ccc3nnn(CCF)c3c2)c2c(OC)nc(N[C@@H]3CCN(C(=O)C([2H])([2H])[2H])CC3(F)F)nn12] |
| 739 | 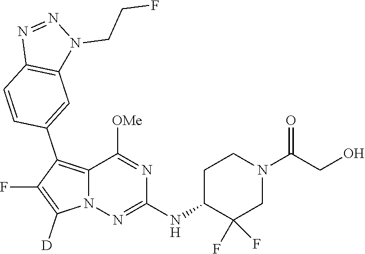 [SMILES: [2H]c1c(F)c(-c2ccc3nnn(CCF)c3c2)c2c(OC)nc(N[C@@H]3CCN(C(=O)CO)CC3(F)F)nn12] |
| 740 | 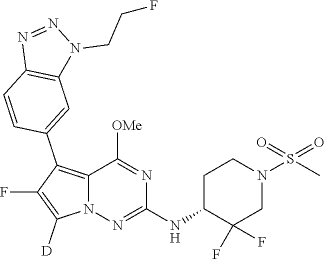 [SMILES: [2H]c1c(F)c(-c2ccc3nnn(CCF)c3c2)c2c(OC)nc(N[C@@H]3CCN(S(C)(=O)=O)CC3(F)F)nn12] |
| 741 | 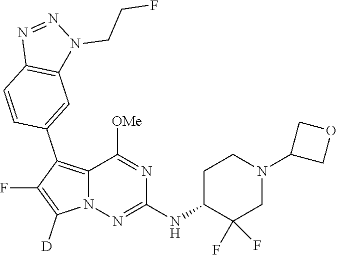 [SMILES: [2H]c1c(F)c(-c2ccc3nnn(CCF)c3c2)c2c(OC)nc(N[C@@H]3CCN(C4COC4)CC3(F)F)nn12] |
| 742 | 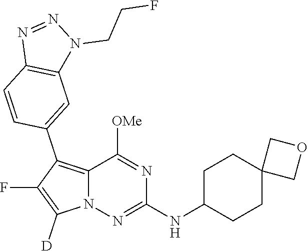 [SMILES: [2H]c1c(F)c(-c2ccc3nnn(CCF)c3c2)c2c(OC)nc(NC3CCC4(CC3)COC4)nn12] |
| 743 | 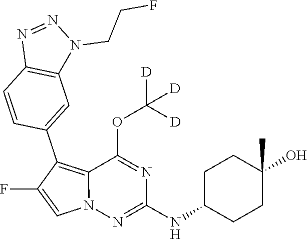 [SMILES: [2H]C([2H])([2H])Oc1nc(N[C@H]2CC[C@@](C)(O)CC2)nn2cc(F)c(-c3ccc4nnn(CCF)c4c3)c12] |
| 744 | 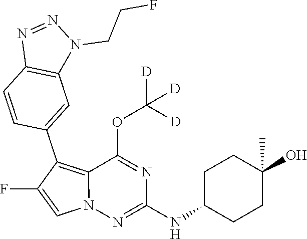 [SMILES: [2H]C([2H])([2H])Oc1nc(N[C@H]2CC[C@](C)(O)CC2)nn2cc(F)c(-c3ccc4nnn(CCF)c4c3)c12] |
| 745 | 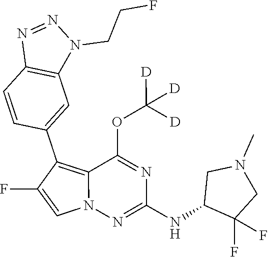 [SMILES: [2H]C([2H])([2H])Oc1nc(N[C@@H]2CN(C)CC2(F)F)nn2cc(F)c(-c3ccc4nnn(CCF)c4c3)c12] |
| 746 | 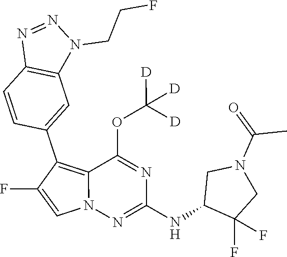 [SMILES: [2H]C([2H])([2H])Oc1nc(N[C@@H]2CN(C(C)=O)CC2(F)F)nn2cc(F)c(-c3ccc4nnn(CCF)c4c3)c12] |
| 747 | 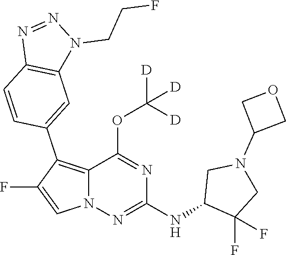 [SMILES: [2H]C([2H])([2H])Oc1nc(N[C@@H]2CN(C3COC3)CC2(F)F)nn2cc(F)c(-c3ccc4nnn(CCF)c4c3)c12] |
| 748 | 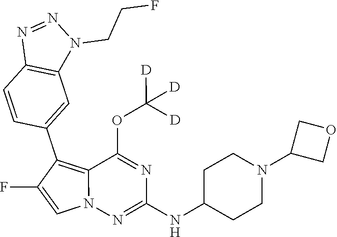 [SMILES: [2H]C([2H])([2H])Oc1nc(NC2CCN(C3COC3)CC2)nn2cc(F)c(-c3ccc4nnn(CCF)c4c3)c12] |
| 749 | 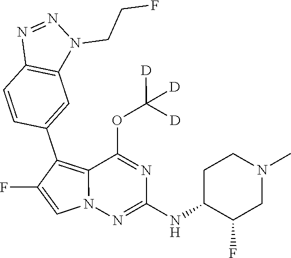 [SMILES: [2H]C([2H])([2H])Oc1nc(N[C@@H]2CCN(C)C[C@@H]2F)nn2cc(F)c(-c3ccc4nnn(CCF)c4c3)c12] |
| 750 | 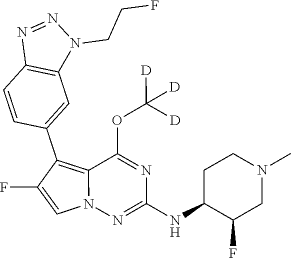 [SMILES: [2H]C([2H])([2H])Oc1nc(N[C@H]2CCN(C)C[C@H]2F)nn2cc(F)c(-c3ccc4nnn(CCF)c4c3)c12] |
| 751 | 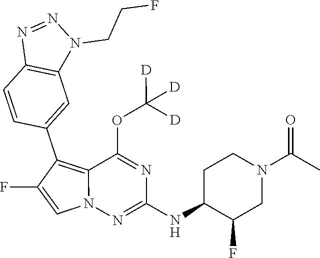 [SMILES: [2H]C([2H])([2H])Oc1nc(N[C@H]2CCN(C(C)=O)C[C@H]2F)nn2cc(F)c(-c3ccc4nnn(CCF)c4c3)c12] |
| 752 | 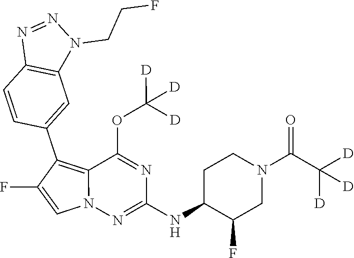 [SMILES: [2H]C([2H])([2H])Oc1nc(N[C@H]2CCN(C(=O)C([2H])([2H])[2H])C[C@H]2F)nn2cc(F)c(-c3ccc4nnn(CCF)c4c3)c12] |
| 753 | 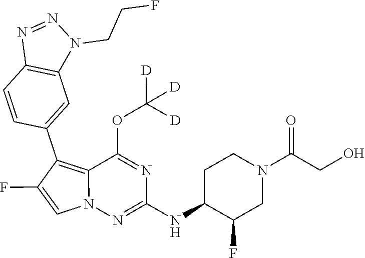 [SMILES: [2H]C([2H])([2H])Oc1nc(N[C@H]2CCN(C(=O)CO)C[C@H]2F)nn2cc(F)c(-c3ccc4nnn(CCF)c4c3)c12] |
| 754 | 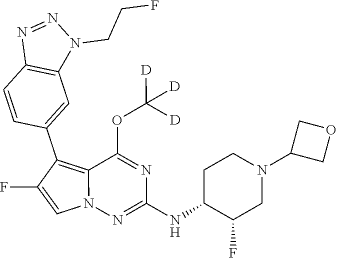 [SMILES: [2H]C([2H])([2H])Oc1nc(N[C@@H]2CCN(C3COC3)C[C@@H]2F)nn2cc(F)c(-c3ccc4nnn(CCF)c4c3)c12] |
| 755 | 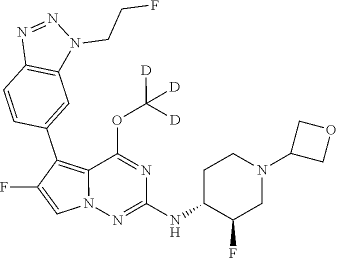 [SMILES: [2H]C([2H])([2H])Oc1nc(N[C@@H]2CCN(C3COC3)C[C@H]2F)nn2cc(F)c(-c3ccc4nnn(CCF)c4c3)c12] |
| 756 | 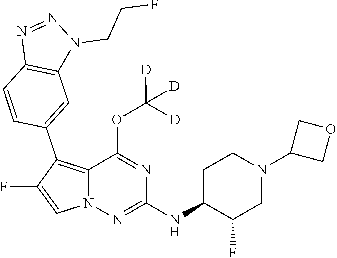 [SMILES: [2H]C([2H])([2H])Oc1nc(N[C@H]2CCN(C3COC3)C[C@@H]2F)nn2cc(F)c(-c3ccc4nnn(CCF)c4c3)c12] |
| 757 | 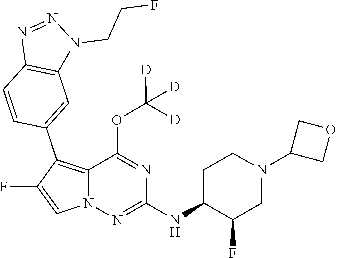 [SMILES: [2H]C([2H])([2H])Oc1nc(N[C@H]2CCN(C3COC3)C[C@H]2F)nn2cc(F)c(-c3ccc4nnn(CCF)c4c3)c12] |
| 758 | 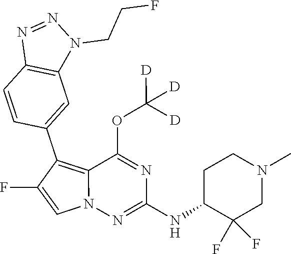 [SMILES: [2H]C([2H])([2H])Oc1nc(N[C@@H]2CCN(C)CC2(F)F)nn2cc(F)c(-c3ccc4nnn(CCF)c4c3)c12] |
| 759 | 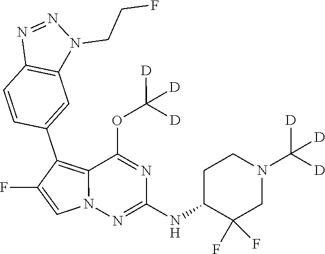 [SMILES: [2H]C([2H])([2H])Oc1nc(N[C@@H]2CCN(C([2H])([2H])[2H])CC2(F)F)nn2cc(F)c(-c3ccc4nnn(CCF)c4c3)c12] |
| 760 | 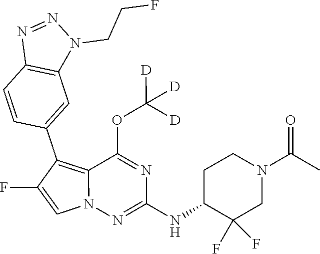 [SMILES: [2H]C([2H])([2H])Oc1nc(N[C@@H]2CCN(C(C)=O)CC2(F)F)nn2cc(F)c(-c3ccc4nnn(CCF)c4c3)c12] |
| 761 | 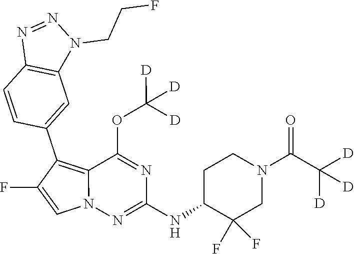 [SMILES: [2H]C([2H])([2H])Oc1nc(N[C@@H]2CCN(C(=O)C([2H])([2H])[2H])CC2(F)F)nn2cc(F)c(-c3ccc4nnn(CCF)c4c3)c12] |
| 762 | 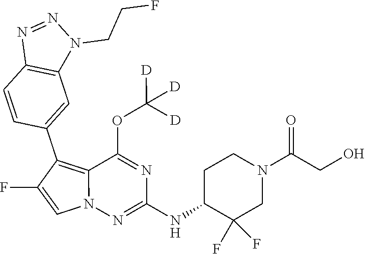 [SMILES: [2H]C([2H])([2H])Oc1nc(N[C@@H]2CCN(C(=O)CO)CC2(F)F)nn2cc(F)c(-c3ccc4nnn(CCF)c4c3)c12] |
| 763 | 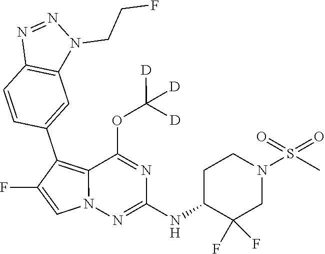 [SMILES: [2H]C([2H])([2H])Oc1nc(N[C@@H]2CCN(S(C)(=O)=O)CC2(F)F)nn2cc(F)c(-c3ccc4nnn(CCF)c4c3)c12] |
| 764 | 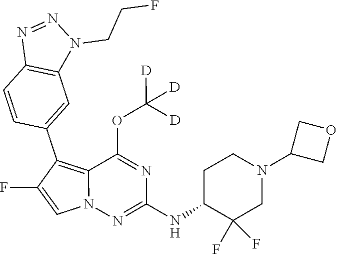 [SMILES: [2H]C([2H])([2H])Oc1nc(N[C@@H]2CCN(C3COC3)CC2(F)F)nn2cc(F)c(-c3ccc4nnn(CCF)c4c3)c12] |
| 765 | 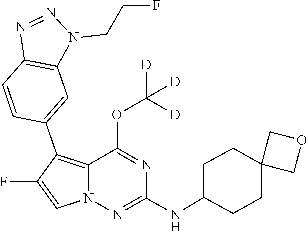 [SMILES: [2H]C([2H])([2H])Oc1nc(NC2CCC3(CC2)COC3)nn2cc(F)c(-c3ccc4nnn(CCF)c4c3)c12] |
| 766 | 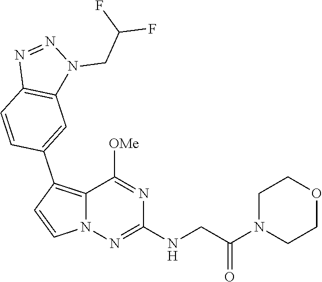 |
| 767 | 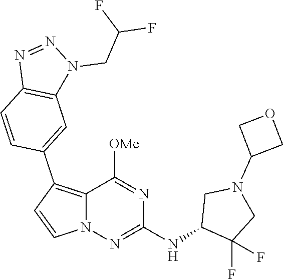 [SMILES: COc1nc(N[C@@H]2CN(C3COC3)CC2(F)F)nn2ccc(-c3ccc4nnn(CC(F)F)c4c3)c12] |
| 768 | 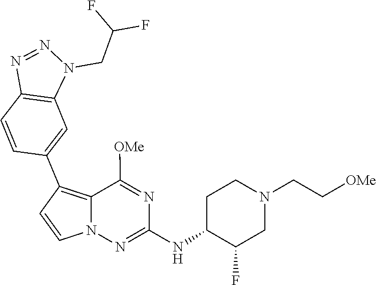 [SMILES: COCCN1CC[C@@H](Nc2nc(OC)c3c(-c4ccc5nnn(CC(F)F)c5c4)ccn3n2)[C@@H](F)C1] |
| 769 | 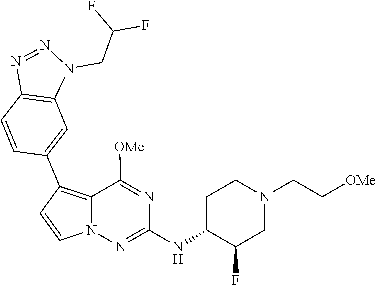 [SMILES: COCCN1CC[C@@H](Nc2nc(OC)c3c(-c4ccc5nnn(CC(F)F)c5c4)ccn3n2)[C@H](F)C1] |
| 770 | 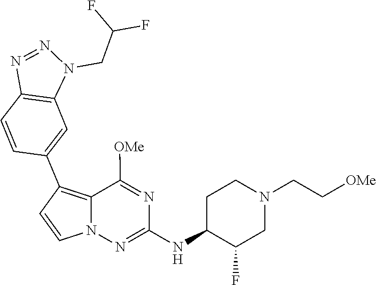 [SMILES: COCCN1CC[C@H](Nc2nc(OC)c3c(-c4ccc5nnn(CC(F)F)c5c4)ccn3n2)[C@@H](F)C1] |
| 771 | 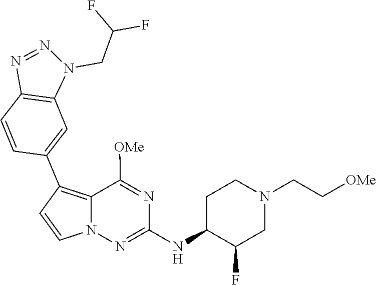 [SMILES: COCCN1CC[C@H](Nc2nc(OC)c3c(-c4ccc5nnn(CC(F)F)c5c4)ccn3n2)[C@H](F)C1] |
| 772 | 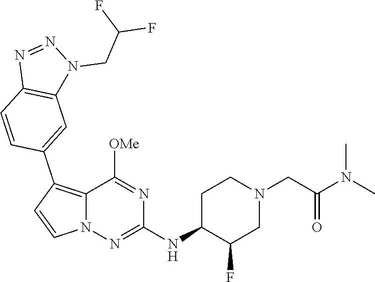 [SMILES: COc1nc(N[C@H]2CCN(CC(=O)N(C)C)C[C@H]2F)nn2ccc(-c3ccc4nnn(CC(F)F)c4c3)c12] |
| 773 | 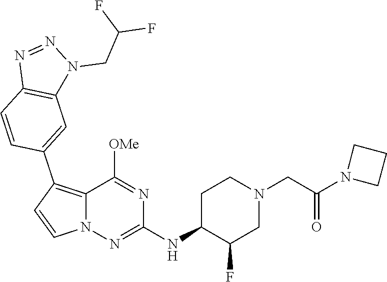 [SMILES: COc1nc(N[C@H]2CCN(CC(=O)N3CCC3)C[C@H]2F)nn2ccc(-c3ccc4nnn(CC(F)F)c4c3)c12] |
| 774 | 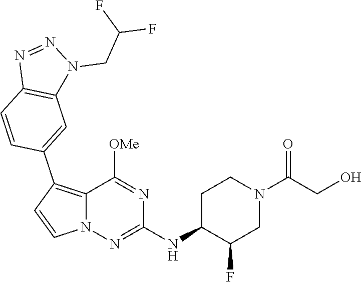 [SMILES: COc1nc(N[C@H]2CCN(C(=O)CO)C[C@H]2F)nn2ccc(-c3ccc4nnn(CC(F)F)c4c3)c12] |
| 775 | 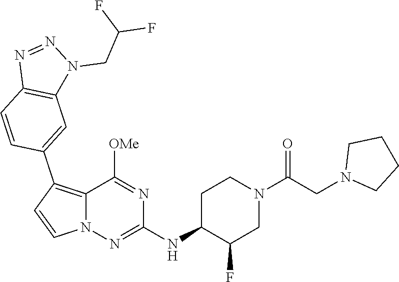 [SMILES: COc1nc(N[C@H]2CCN(C(=O)CN3CCCC3)C[C@H]2F)nn2ccc(-c3ccc4nnn(CC(F)F)c4c3)c12] |
| 776 | 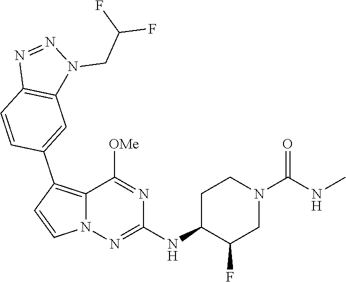 [SMILES: CNC(=O)N1CC[C@H](Nc2nc(OC)c3c(-c4ccc5nnn(CC(F)F)c5c4)ccn3n2)[C@H](F)C1] |
| 777 | 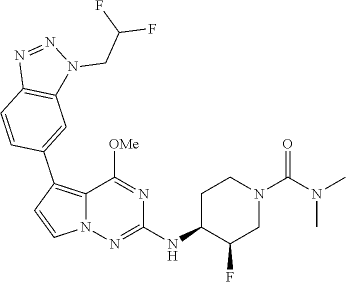 [SMILES: COc1nc(N[C@H]2CCN(C(=O)N(C)C)C[C@H]2F)nn2ccc(-c3ccc4nnn(CC(F)F)c4c3)c12] |
| 778 | 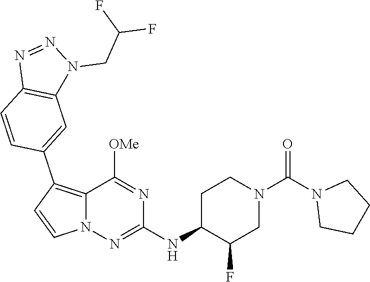 [SMILES: COc1nc(N[C@H]2CCN(C(=O)N3CCCC3)C[C@H]2F)nn2ccc(-c3ccc4nnn(CC(F)F)c4c3)c12] |
| 779 | 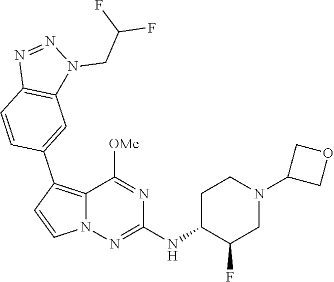 [SMILES: COc1nc(N[C@@H]2CCN(C3COC3)C[C@H]2F)nn2ccc(-c3ccc4nnn(CC(F)F)c4c3)c12] |
| 780 | 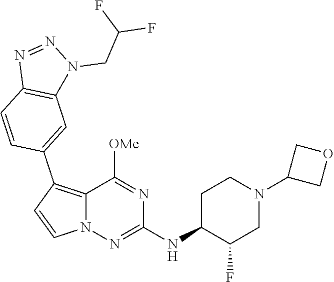 [SMILES: COc1nc(N[C@H]2CCN(C3COC3)C[C@@H]2F)nn2ccc(-c3ccc4nnn(CC(F)F)c4c3)c12] |
| 781 | 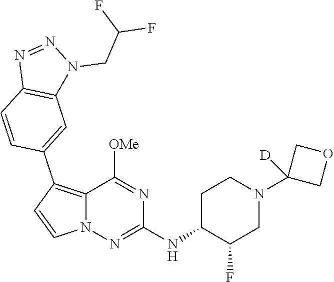 [SMILES: [2H]C1(N2CC[C@@H](Nc3nc(OC)c4c(-c5ccc6nnn(CC(F)F)c6c5)ccn4n3)[C@@H](F)C2)COC1] |
| 782 | 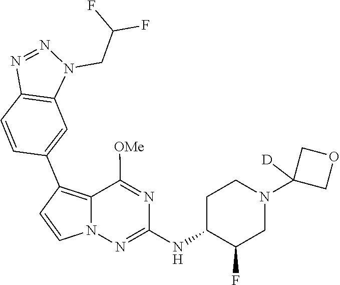 [SMILES: [2H]C1(N2CC[C@@H](Nc3nc(OC)c4c(-c5ccc6nnn(CC(F)F)c6c5)ccn4n3)[C@H](F)C2)COC1] |
| 783 | 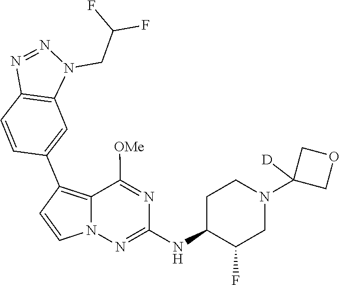 [SMILES: [2H]C1(N2CC[C@H](Nc3nc(OC)c4c(-c5ccc6nnn(CC(F)F)c6c5)ccn4n3)[C@@H](F)C2)COC1] |
| 784 | 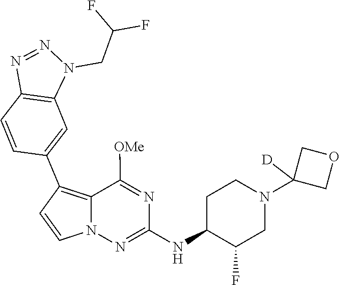 [SMILES: [2H]C1(N2CC[C@H](Nc3nc(OC)c4c(-c5ccc6nnn(CC(F)F)c6c5)ccn4n3)[C@@H](F)C2)COC1] |
| 785 | 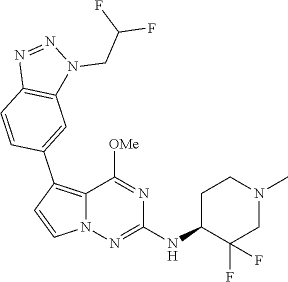 [SMILES: COc1nc(N[C@H]2CCN(C)CC2(F)F)nn2ccc(-c3ccc4nnn(CC(F)F)c4c3)c12] |
| 786 | 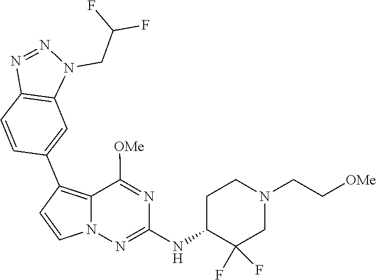 [SMILES: COCCN1CC[C@@H](Nc2nc(OC)c3c(-c4ccc5nnn(CC(F)F)c5c4)ccn3n2)C(F)(F)C1] |
| 787 | 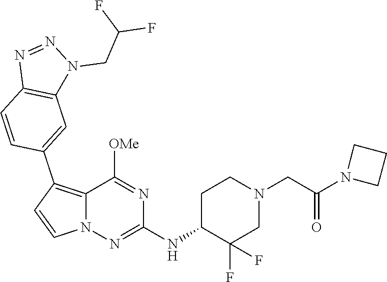 [SMILES: COc1nc(N[C@@H]2CCN(CC(=O)N3CCC3)CC2(F)F)nn2ccc(-c3ccc4nnn(CC(F)F)c4c3)c12] |
| 788 | 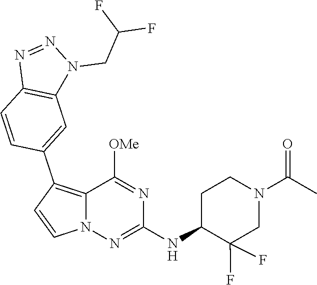 [SMILES: COc1nc(N[C@H]2CCN(C(C)=O)CC2(F)F)nn2ccc(-c3ccc4nnn(CC(F)F)c4c3)c12] |
| 789 | 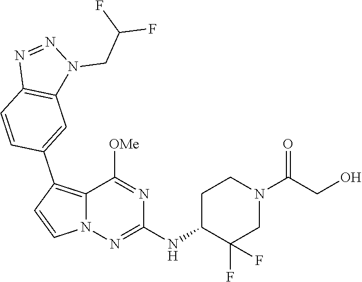 [SMILES: COc1nc(N[C@@H]2CCN(C(=O)CO)CC2(F)F)nn2ccc(-c3ccc4nnn(CC(F)F)c4c3)c12] |
| 790 | 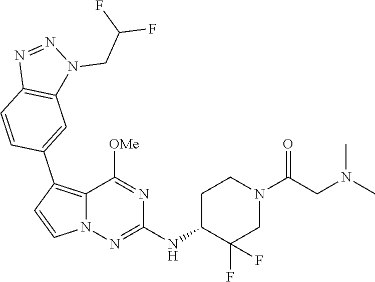 [SMILES: COc1nc(N[C@@H]2CCN(C(=O)CN(C)C)CC2(F)F)nn2ccc(-c3ccc4nnn(CC(F)F)c4c3)c12] |
| 791 | 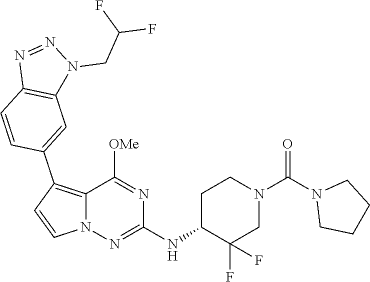 [SMILES: COc1nc(N[C@@H]2CCN(C(=O)N3CCCC3)CC2(F)F)nn2ccc(-c3ccc4nnn(CC(F)F)c4c3)c12] |
| 792 | 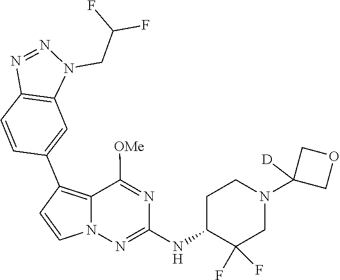 [SMILES: [2H]C1(N2CC[C@@H](Nc3nc(OC)c4c(-c5ccc6nnn(CC(F)F)c6c5)ccn4n3)C(F)(F)C2)COC1] |
| 793 | 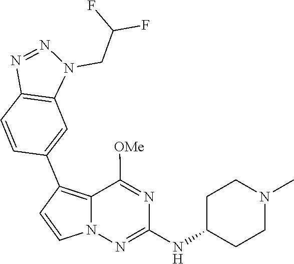 |
| 794 | 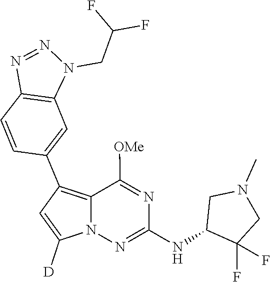 [SMILES: [2H]c1cc(-c2ccc3nnn(CC(F)F)c3c2)c2c(OC)nc(N[C@@H]3CN(C)CC3(F)F)nn12] |
| 795 | 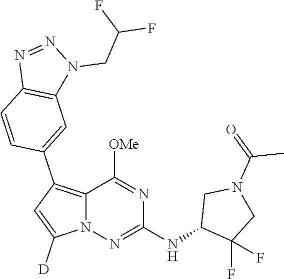 [SMILES: [2H]c1cc(-c2ccc3nnn(CC(F)F)c3c2)c2c(OC)nc(N[C@@H]3CN(C(C)=O)CC3(F)F)nn12] |
| 796 | 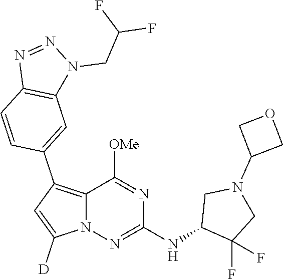 [SMILES: [2H]c1cc(-c2ccc3nnn(CC(F)F)c3c2)c2c(OC)nc(N[C@@H]3CN(C4COC4)CC3(F)F)nn12] |
| 797 | 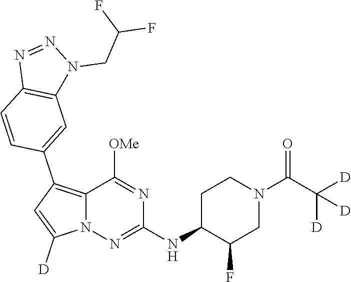 [SMILES: [2H]c1cc(-c2ccc3nnn(CC(F)F)c3c2)c2c(OC)nc(N[C@H]3CCN(C(=O)C([2H])([2H])[2H])C[C@H]3F)nn12] |
| 798 | 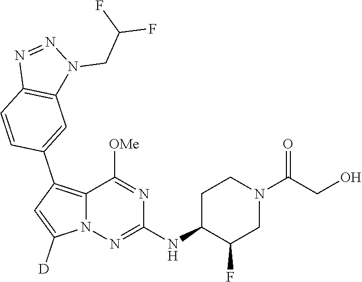 [SMILES: [2H]c1cc(-c2ccc3nnn(CC(F)F)c3c2)c2c(OC)nc(N[C@H]3CCN(C(=O)CO)C[C@H]3F)nn12] |
| 799 | 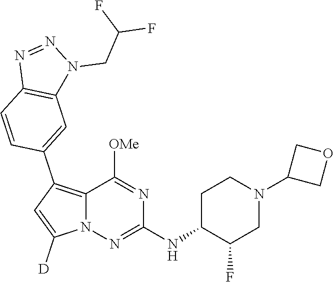 [SMILES: [2H]c1cc(-c2ccc3nnn(CC(F)F)c3c2)c2c(OC)nc(N[C@@H]3CCN(C4COC4)C[C@@H]3F)nn12] |
| 800 | 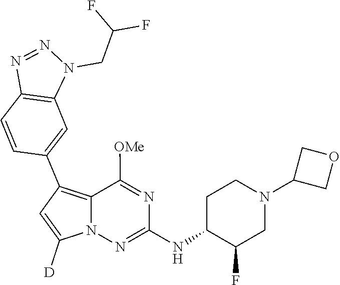 [SMILES: [2H]c1cc(-c2ccc3nnn(CC(F)F)c3c2)c2c(OC)nc(N[C@@H]3CCN(C4COC4)C[C@H]3F)nn12] |
| 801 | 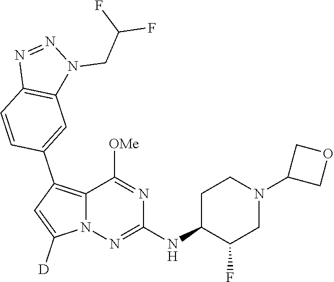 [SMILES: [2H]c1cc(-c2ccc3nnn(CC(F)F)c3c2)c2c(OC)nc(N[C@H]3CCN(C4COC4)C[C@@H]3F)nn12] |
| 802 | 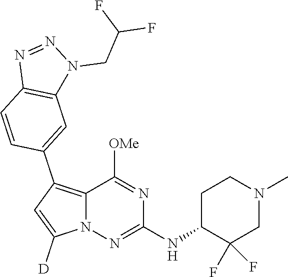 [SMILES: [2H]c1cc(-c2ccc3nnn(CC(F)F)c3c2)c2c(OC)nc(N[C@@H]3CCN(C)CC3(F)F)nn12] |
| 803 | 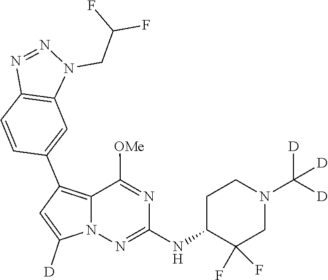 [SMILES: [2H]c1cc(-c2ccc3nnn(CC(F)F)c3c2)c2c(OC)nc(N[C@@H]3CCN(C([2H])([2H])[2H])CC3(F)F)nn12] |
| 804 | 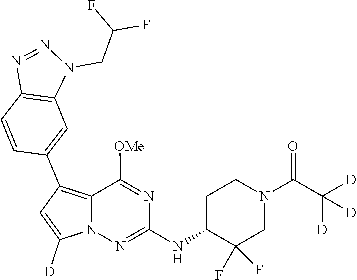 [SMILES: [2H]c1cc(-c2ccc3nnn(CC(F)F)c3c2)c2c(OC)nc(N[C@@H]3CCN(C(=O)C([2H])([2H])[2H])CC3(F)F)nn12] |
| 805 | 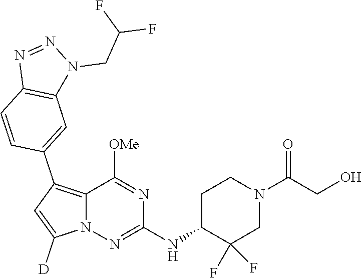 [SMILES: [2H]c1cc(-c2ccc3nnn(CC(F)F)c3c2)c2c(OC)nc(N[C@@H]3CCN(C(=O)CO)CC3(F)F)nn12] |
| 806 | 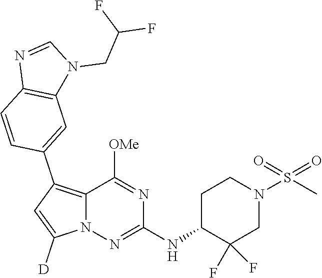 [SMILES: [2H]c1cc(-c2ccc3ncn(CC(F)F)c3c2)c2c(OC)nc(N[C@@H]3CCN(S(C)(=O)=O)CC3(F)F)nn12] |
| 807 | 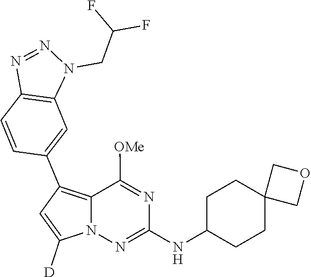 [SMILES: [2H]c1cc(-c2ccc3nnn(CC(F)F)c3c2)c2c(OC)nc(NC3CCC4(CC3)COC4)nn12] |
| 808 | 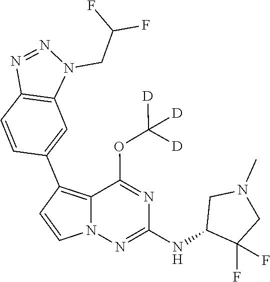 [SMILES: [2H]C([2H])([2H])Oc1nc(N[C@@H]2CN(C)CC2(F)F)nn2ccc(-c3ccc4nnn(CC(F)F)c4c3)c12] |
| 809 | 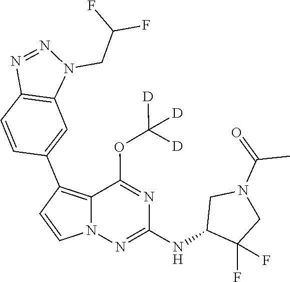 [SMILES: [2H]C([2H])([2H])Oc1nc(N[C@@H]2CN(C(C)=O)CC2(F)F)nn2ccc(-c3ccc4nnn(CC(F)F)c4c3)c12] |
| 810 | 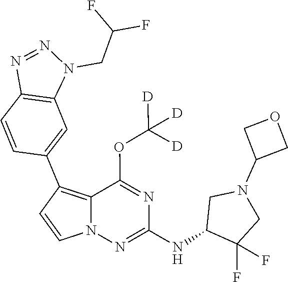 [SMILES: [2H]C([2H])([2H])Oc1nc(N[C@@H]2CN(C3COC3)CC2(F)F)nn2ccc(-c3ccc4nnn(CC(F)F)c4c3)c12] |
| 811 | 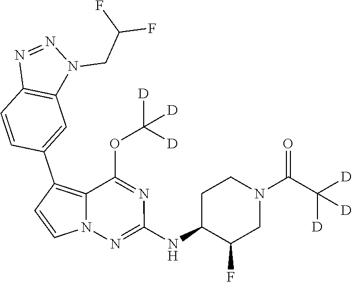 [SMILES: [2H]C([2H])([2H])Oc1nc(N[C@H]2CCN(C(=O)C([2H])([2H])[2H])C[C@H]2F)nn2ccc(-c3ccc4nnn(CC(F)F)c4c3)c12] |
| 812 | 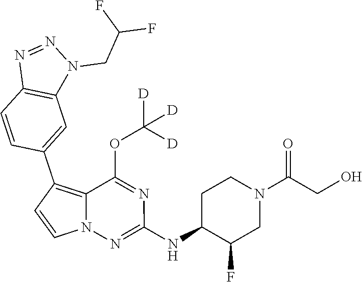 [SMILES: [2H]C([2H])([2H])Oc1nc(N[C@H]2CCN(C(=O)CO)C[C@H]2F)nn2ccc(-c3ccc4nnn(CC(F)F)c4c3)c12] |
| 813 | 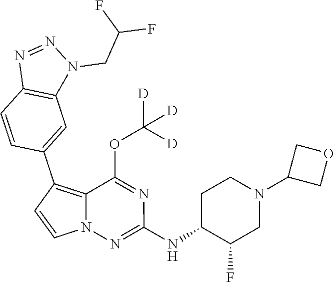 [SMILES: [2H]C([2H])([2H])Oc1nc(N[C@@H]2CCN(C3COC3)C[C@@H]2F)nn2ccc(-c3ccc4nnn(CC(F)F)c4c3)c12] |
| 814 | 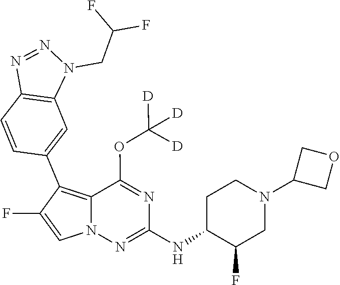 [SMILES: [2H]C([2H])([2H])Oc1nc(N[C@@H]2CCN(C3COC3)C[C@H]2F)nn2cc(F)c(-c3ccc4nnn(CC(F)F)c4c3)c12] |
| 815 | 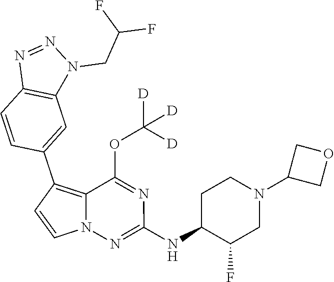 [SMILES: [2H]C([2H])([2H])Oc1nc(N[C@H]2CCN(C3COC3)C[C@@H]2F)nn2ccc(-c3ccc4nnn(CC(F)F)c4c3)c12] |
| 816 | 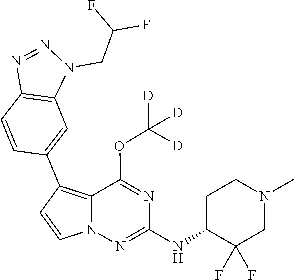 [SMILES: [2H]C([2H])([2H])Oc1nc(N[C@@H]2CCN(C)CC2(F)F)nn2ccc(-c3ccc4nnn(CC(F)F)c4c3)c12] |
| 817 | 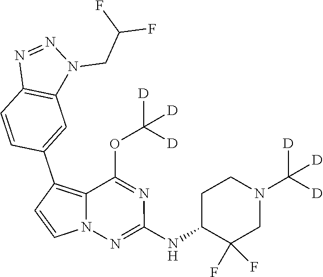 [SMILES: [2H]C([2H])([2H])Oc1nc(N[C@@H]2CCN(C([2H])([2H])[2H])CC2(F)F)nn2ccc(-c3ccc4nnn(CC(F)F)c4c3)c12] |
| 818 | 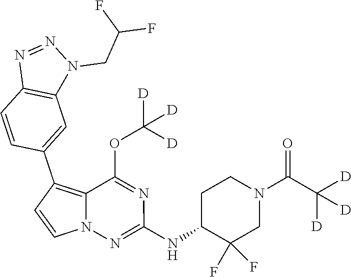 [SMILES: [2H]C([2H])([2H])Oc1nc(N[C@@H]2CCN(C(=O)C([2H])([2H])[2H])CC2(F)F)nn2ccc(-c3ccc4nnn(CC(F)F)c4c3)c12] |
| 819 | 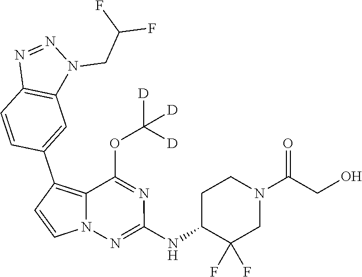 [SMILES: [2H]C([2H])([2H])Oc1nc(N[C@@H]2CCN(C(=O)CO)CC2(F)F)nn2ccc(-c3ccc4nnn(CC(F)F)c4c3)c12] |
| 820 | 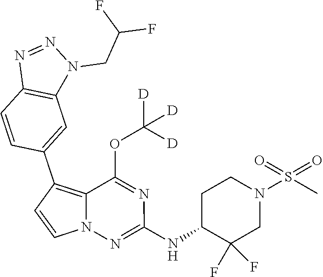 [SMILES: [2H]C([2H])([2H])Oc1nc(N[C@@H]2CCN(S(C)(=O)=O)CC2(F)F)nn2ccc(-c3ccc4nnn(CC(F)F)c4c3)c12] |
| 821 | 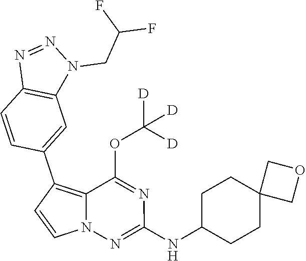 [SMILES: [2H]C([2H])([2H])Oc1nc(NC2CCC3(CC2)COC3)nn2ccc(-c3ccc4nnn(CC(F)F)c4c3)c12] |
| 822 | 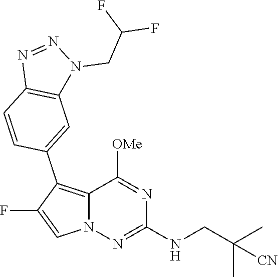 [SMILES: COc1nc(NCC(C)(C)C#N)nn2cc(F)c(-c3ccc4nnn(CC(F)F)c4c3)c12] |
| 823 | 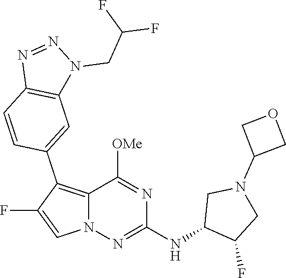 [SMILES: COc1nc(N[C@@H]2CN(C3COC3)C[C@@H]2F)nn2cc(F)c(-c3ccc4nnn(CC(F)F)c4c3)c12] |
| 824 | 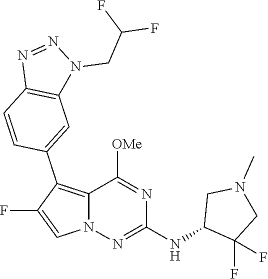 [SMILES: COc1nc(N[C@@H]2CN(C)CC2(F)F)nn2cc(F)c(-c3ccc4nnn(CC(F)F)c4c3)c12] |
| 825 | 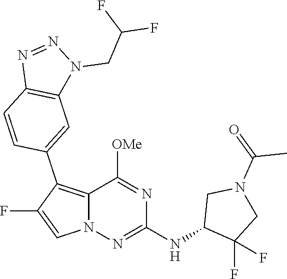 [SMILES: COc1nc(N[C@@H]2CN(C(C)=O)CC2(F)F)nn2cc(F)c(-c3ccc4nnn(CC(F)F)c4c3)c12] |
| 826 | 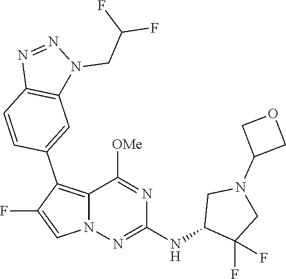 [SMILES: COc1nc(N[C@@H]2CN(C3COC3)CC2(F)F)nn2cc(F)c(-c3ccc4nnn(CC(F)F)c4c3)c12] |
| 827 | 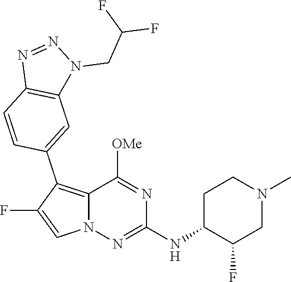 [SMILES: COc1nc(N[C@@H]2CCN(C)C[C@@H]2F)nn2cc(F)c(-c3ccc4nnn(CC(F)F)c4c3)c12] |
| 828 | 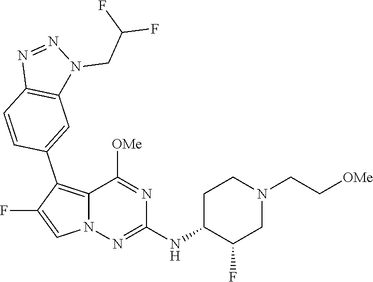 [SMILES: COCCN1CC[C@@H](Nc2nc(OC)c3c(-c4ccc5nnn(CC(F)F)c5c4)c(F)cn3n2)[C@@H](F)C1] |
| 829 | 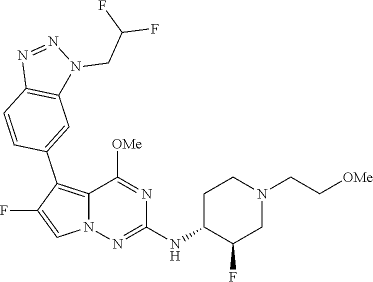 [SMILES: COCCN1CC[C@@H](Nc2nc(OC)c3c(-c4ccc5nnn(CC(F)F)c5c4)c(F)cn3n2)[C@H](F)C1] |
| 830 | 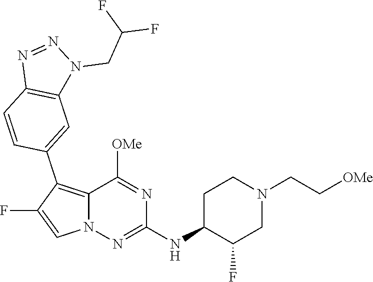 [SMILES: COCCN1CC[C@H](Nc2nc(OC)c3c(-c4ccc5nnn(CC(F)F)c5c4)c(F)cn3n2)[C@@H](F)C1] |
| 831 | 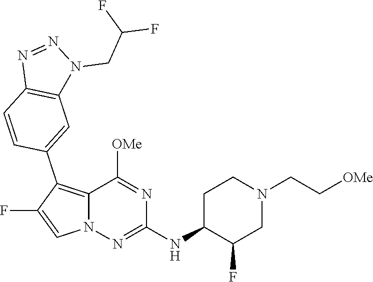 [SMILES: COCCN1CC[C@H](Nc2nc(OC)c3c(-c4ccc5nnn(CC(F)F)c5c4)c(F)cn3n2)[C@H](F)C1] |
| 832 | 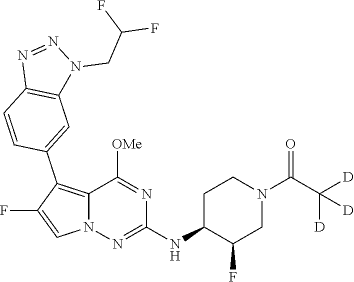 [SMILES: [2H]C([2H])([2H])C(=O)N1CC[C@H](Nc2nc(OC)c3c(-c4ccc5nnn(CC(F)F)c5c4)c(F)cn3n2)[C@H](F)C1] |
| 833 | 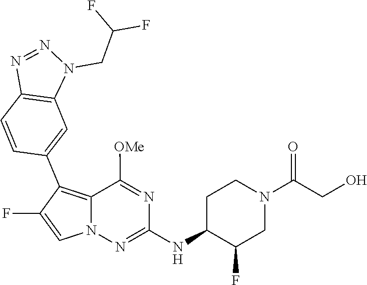 [SMILES: COc1nc(N[C@H]2CCN(C(=O)CO)C[C@H]2F)nn2cc(F)c(-c3ccc4nnn(CC(F)F)c4c3)c12] |
| 834 | 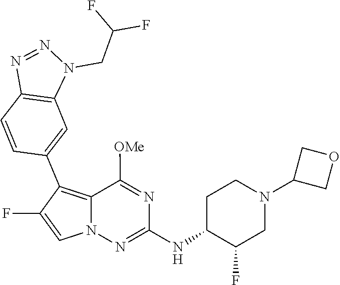 [SMILES: COc1nc(N[C@@H]2CCN(C3COC3)C[C@@H]2F)nn2cc(F)c(-c3ccc4nnn(CC(F)F)c4c3)c12] |
| 835 | 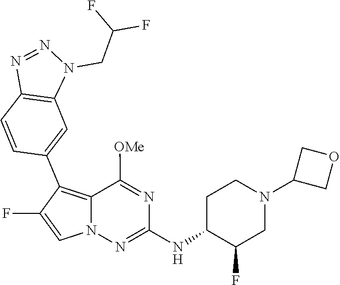 [SMILES: COc1nc(N[C@@H]2CCN(C3COC3)C[C@H]2F)nn2cc(F)c(-c3ccc4nnn(CC(F)F)c4c3)c12] |
| 836 | 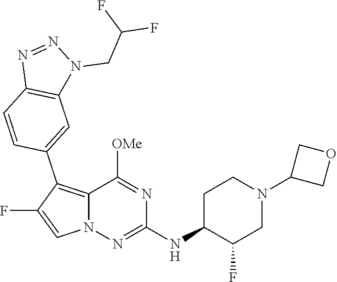 [SMILES: COc1nc(N[C@H]2CCN(C3COC3)C[C@@H]2F)nn2cc(F)c(-c3ccc4nnn(CC(F)F)c4c3)c12] |
| 837 | 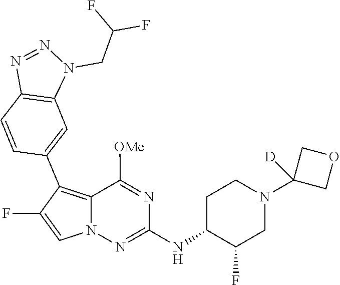 [SMILES: [2H]C1(N2CC[C@@H](Nc3nc(OC)c4c(-c5ccc6nnn(CC(F)F)c6c5)c(F)cn4n3)[C@@H](F)C2)COC1] |
| 838 | 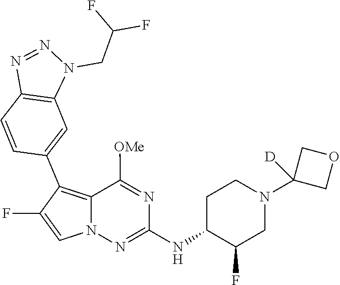 [SMILES: [2H]C1(N2CC[C@@H](Nc3nc(OC)c4c(-c5ccc6nnn(CC(F)F)c6c5)c(F)cn4n3)[C@H](F)C2)COC1] |
| 839 | 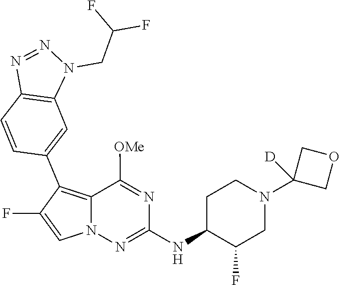 [SMILES: [2H]C1(N2CC[C@H](Nc3nc(OC)c4c(-c5ccc6nnn(CC(F)F)c6c5)c(F)cn4n3)[C@@H](F)C2)COC1] |
| 840 | 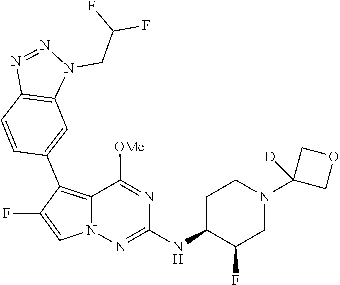 [SMILES: [2H]C1(N2CC[C@H](Nc3nc(OC)c4c(-c5ccc6nnn(CC(F)F)c6c5)c(F)cn4n3)[C@H](F)C2)COC1] |
| 841 | 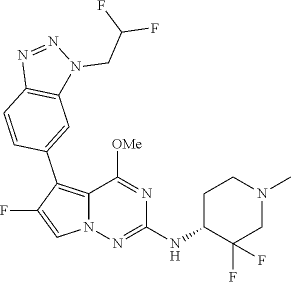 [SMILES: COc1nc(N[C@@H]2CCN(C)CC2(F)F)nn2cc(F)c(-c3ccc4nnn(CC(F)F)c4c3)c12] |
| 842 | 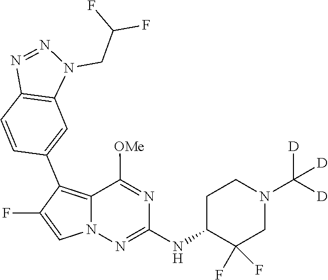 [SMILES: [2H]C([2H])([2H])N1CC[C@@H](Nc2nc(OC)c3c(-c4ccc5nnn(CC(F)F)c5c4)c(F)cn3n2)C(F)(F)C1] |
| 843 | 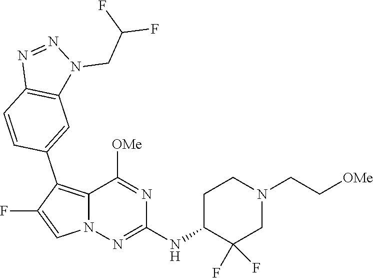 [SMILES: COCCN1CC[C@@H](Nc2nc(OC)c3c(-c4ccc5nnn(CC(F)F)c5c4)c(F)cn3n2)C(F)(F)C1] |
| 844 | 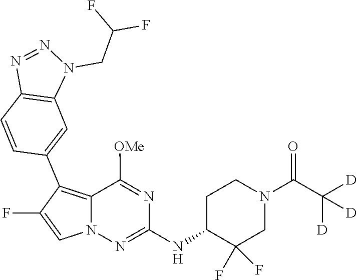 [SMILES: [2H]C([2H])([2H])C(=O)N1CC[C@@H](Nc2nc(OC)c3c(-c4ccc5nnn(CC(F)F)c5c4)c(F)cn3n2)C(F)(F)C1] |
| 845 | 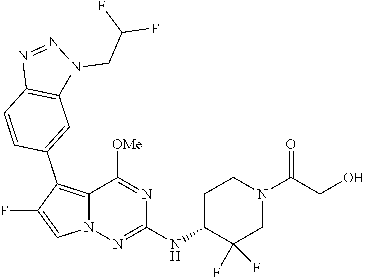 [SMILES: COc1nc(N[C@@H]2CCN(C(=O)CO)CC2(F)F)nn2cc(F)c(-c3ccc4nnn(CC(F)F)c4c3)c12] |
| 846 | 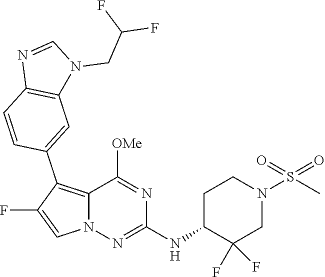 [SMILES: COc1nc(N[C@@H]2CCN(S(C)(=O)=O)CC2(F)F)nn2cc(F)c(-c3ccc4ncn(CC(F)F)c4c3)c12] |
| 847 | 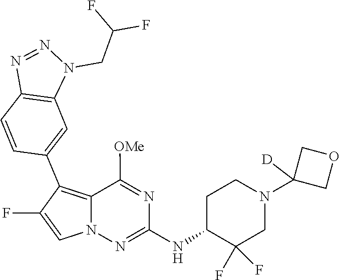 [SMILES: [2H]C1(N2CC[C@@H](Nc3nc(OC)c4c(-c5ccc6nnn(CC(F)F)c6c5)c(F)cn4n3)C(F)(F)C2)COC1] |
| 848 | 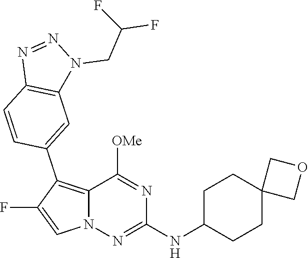 [SMILES: COc1nc(NC2CCC3(CC2)COC3)nn2cc(F)c(-c3ccc4nnn(CC(F)F)c4c3)c12] |
| 849 | 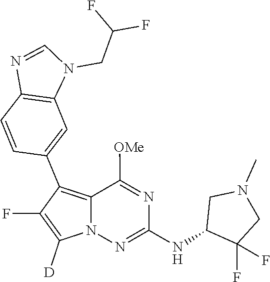 [SMILES: [2H]c1c(F)c(-c2ccc3ncn(CC(F)F)c3c2)c2c(OC)nc(N[C@@H]3CN(C)CC3(F)F)nn12] |
| 850 | 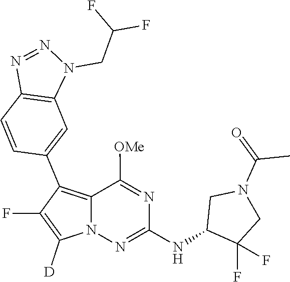 [SMILES: [2H]c1c(F)c(-c2ccc3nnn(CC(F)F)c3c2)c2c(OC)nc(N[C@@H]3CN(C(C)=O)CC3(F)F)nn12] |
| 851 | 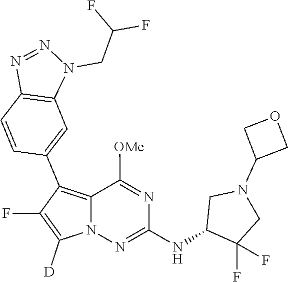 [SMILES: [2H]c1c(F)c(-c2ccc3nnn(CC(F)F)c3c2)c2c(OC)nc(N[C@@H]3CN(C4COC4)CC3(F)F)nn12] |
| 852 | 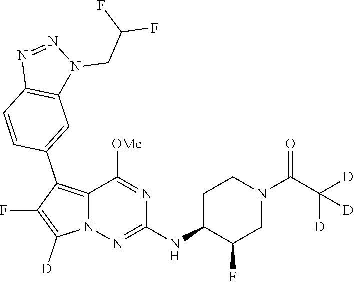 [SMILES: [2H]c1c(F)c(-c2ccc3nnn(CC(F)F)c3c2)c2c(OC)nc(N[C@H]3CCN(C(=O)C([2H])([2H])[2H])C[C@H]3F)nn12] |
| 853 | 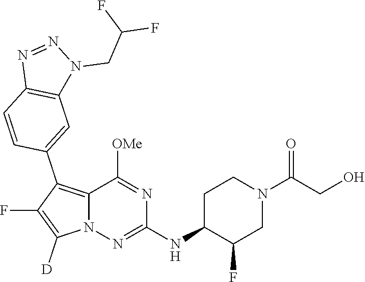 [SMILES: [2H]c1c(F)c(-c2ccc3nnn(CC(F)F)c3c2)c2c(OC)nc(N[C@H]3CCN(C(=O)CO)C[C@H]3F)nn12] |
| 854 | 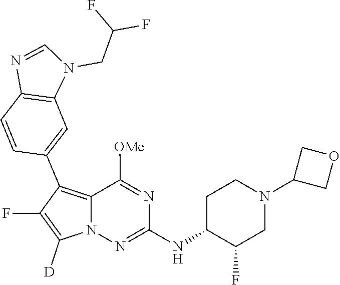 [SMILES: [2H]c1c(F)c(-c2ccc3ncn(CC(F)F)c3c2)c2c(OC)nc(N[C@@H]3CCN(C4COC4)C[C@@H]3F)nn12] |
| 855 | 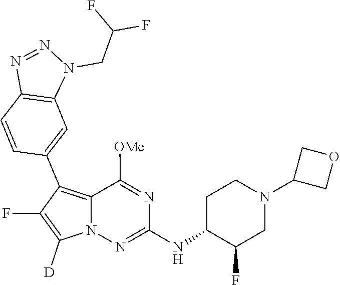 [SMILES: [2H]c1c(F)c(-c2ccc3nnn(CC(F)F)c3c2)c2c(OC)nc(N[C@@H]3CCN(C4COC4)C[C@H]3F)nn12] |
| 856 | 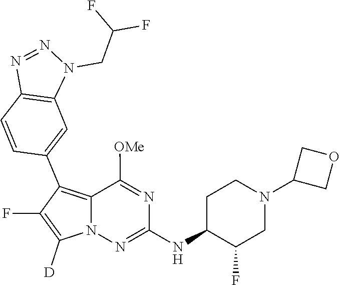 [SMILES: [2H]c1c(F)c(-c2ccc3nnn(CC(F)F)c3c2)c2c(OC)nc(N[C@H]3CCN(C4COC4)C[C@@H]3F)nn12] |
| 857 | 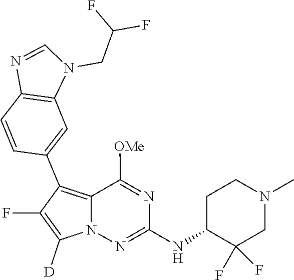 [SMILES: [2H]c1c(F)c(-c2ccc3ncn(CC(F)F)c3c2)c2c(OC)nc(N[C@@H]3CCN(C)CC3(F)F)nn12] |
| 858 | 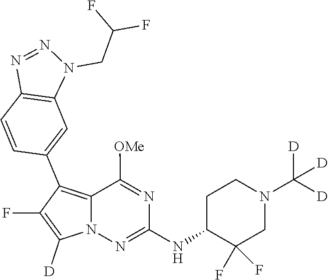 [SMILES: [2H]c1c(F)c(-c2ccc3nnn(CC(F)F)c3c2)c2c(OC)nc(N[C@@H]3CCN(C([2H])([2H])[2H])CC3(F)F)nn12] |
| 859 | 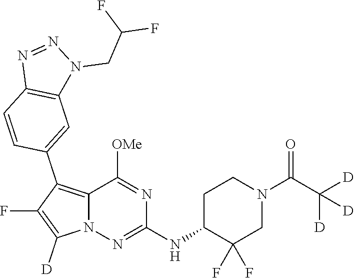 [SMILES: [2H]c1c(F)c(-c2ccc3nnn(CC(F)F)c3c2)c2c(OC)nc(N[C@@H]3CCN(C(=O)C([2H])([2H])[2H])CC3(F)F)nn12] |
| 860 | 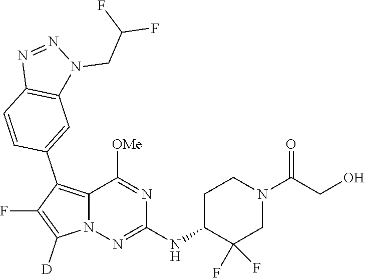 [SMILES: [2H]c1c(F)c(-c2ccc3nnn(CC(F)F)c3c2)c2c(OC)nc(N[C@@H]3CCN(C(=O)CO)CC3(F)F)nn12] |
| 861 | 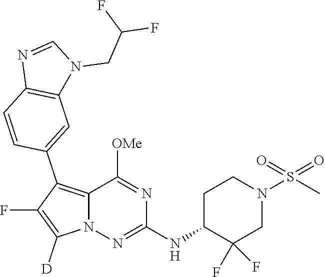 [SMILES: [2H]c1c(F)c(-c2ccc3ncn(CC(F)F)c3c2)c2c(OC)nc(N[C@@H]3CCN(S(C)(=O)=O)CC3(F)F)nn12] |
| 862 | 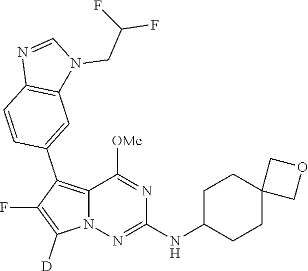 [SMILES: [2H]c1c(F)c(-c2ccc3ncn(CC(F)F)c3c2)c2c(OC)nc(NC3CCC4(CC3)COC4)nn12] |
| 863 | 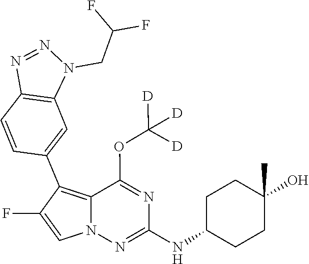 [SMILES: [2H]C([2H])([2H])Oc1nc(N[C@H]2CC[C@@](C)(O)CC2)nn2cc(F)c(-c3ccc4nnn(CC(F)F)c4c3)c12] |
| 864 | 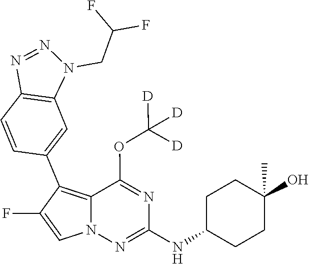 [SMILES: [2H]C([2H])([2H])Oc1nc(N[C@H]2CC[C@](C)(O)CC2)nn2cc(F)c(-c3ccc4nnn(CC(F)F)c4c3)c12] |
| 865 | 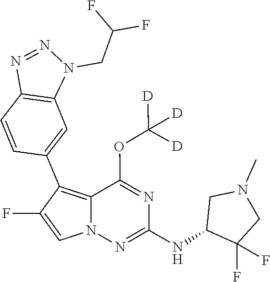 [SMILES: [2H]C([2H])([2H])Oc1nc(N[C@@H]2CN(C)CC2(F)F)nn2cc(F)c(-c3ccc4nnn(CC(F)F)c4c3)c12] |
| 866 | 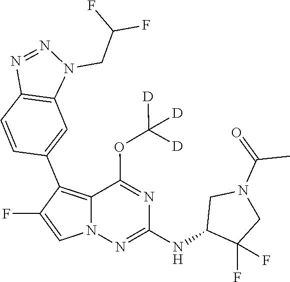 [SMILES: [2H]C([2H])([2H])Oc1nc(N[C@@H]2CN(C(C)=O)CC2(F)F)nn2cc(F)c(-c3ccc4nnn(CC(F)F)c4c3)c12] |
| 867 | 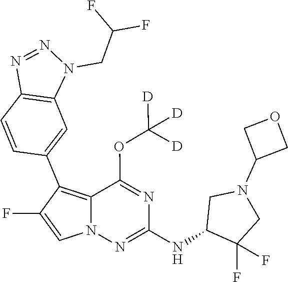 [SMILES: [2H]C([2H])([2H])Oc1nc(N[C@@H]2CN(C3COC3)CC2(F)F)nn2cc(F)c(-c3ccc4nnn(CC(F)F)c4c3)c12] |
| 868 | 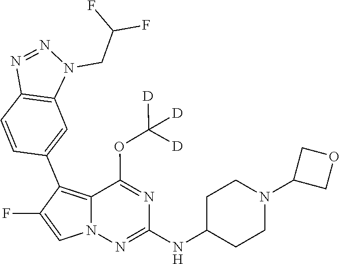 [SMILES: [2H]C([2H])([2H])Oc1nc(NC2CCN(C3COC3)CC2)nn2cc(F)c(-c3ccc4nnn(CC(F)F)c4c3)c12] |
| 869 | 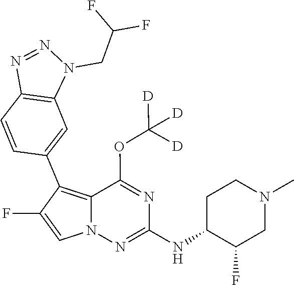 [SMILES: [2H]C([2H])([2H])Oc1nc(N[C@@H]2CCN(C)C[C@@H]2F)nn2cc(F)c(-c3ccc4nnn(CC(F)F)c4c3)c12] |
| 870 | 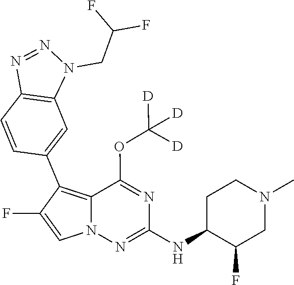 [SMILES: [2H]C([2H])([2H])Oc1nc(N[C@H]2CCN(C)C[C@H]2F)nn2cc(F)c(-c3ccc4nnn(CC(F)F)c4c3)c12] |
| 871 | 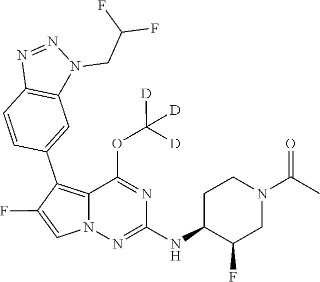 [SMILES: [2H]C([2H])([2H])Oc1nc(N[C@H]2CCN(C(C)=O)C[C@H]2F)nn2cc(F)c(-c3ccc4nnn(CC(F)F)c4c3)c12] |
| 872 | 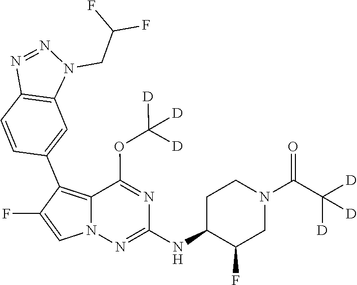 [SMILES: [2H]C([2H])([2H])Oc1nc(N[C@H]2CCN(C(=O)C([2H])([2H])[2H])C[C@H]2F)nn2cc(F)c(-c3ccc4nnn(CC(F)F)c4c3)c12] |
| 873 | 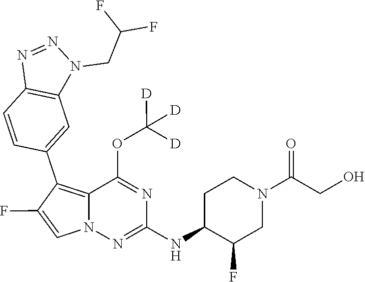 [SMILES: [2H]C([2H])([2H])Oc1nc(N[C@H]2CCN(C(=O)CO)C[C@H]2F)nn2cc(F)c(-c3ccc4nnn(CC(F)F)c4c3)c12] |
| 874 | 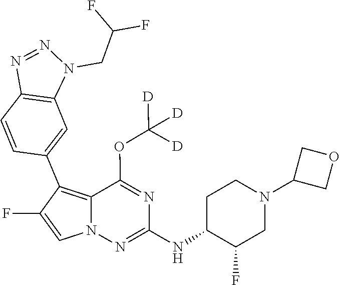 [SMILES: [2H]C([2H])([2H])Oc1nc(N[C@@H]2CCN(C3COC3)C[C@@H]2F)nn2cc(F)c(-c3ccc4nnn(CC(F)F)c4c3)c12] |
| 875 | 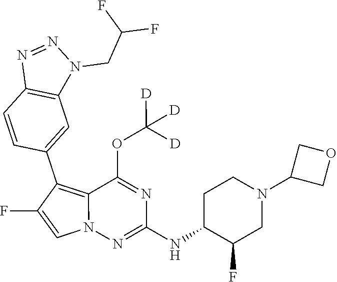 [SMILES: [2H]C([2H])([2H])Oc1nc(N[C@@H]2CCN(C3COC3)C[C@H]2F)nn2cc(F)c(-c3ccc4nnn(CC(F)F)c4c3)c12] |
| 876 | 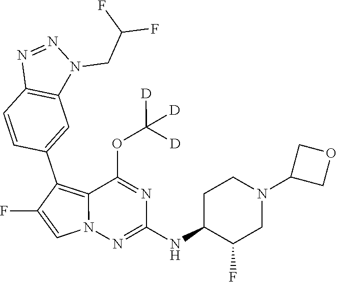 [SMILES: [2H]C([2H])([2H])Oc1nc(N[C@H]2CCN(C3COC3)C[C@@H]2F)nn2cc(F)c(-c3ccc4nnn(CC(F)F)c4c3)c12] |
| 877 | 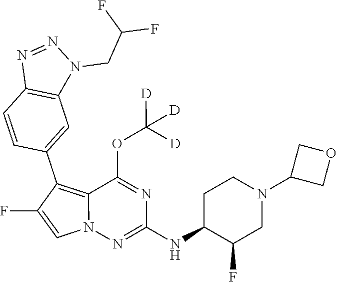 [SMILES: [2H]C([2H])([2H])Oc1nc(N[C@H]2CCN(C3COC3)C[C@H]2F)nn2cc(F)c(-c3ccc4nnn(CC(F)F)c4c3)c12] |
| 878 | 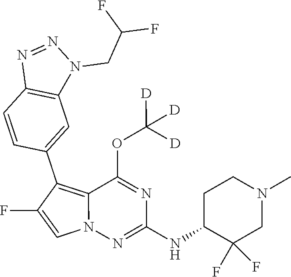 [SMILES: [2H]C([2H])([2H])Oc1nc(N[C@@H]2CCN(C)CC2(F)F)nn2cc(F)c(-c3ccc4nnn(CC(F)F)c4c3)c12] |
| 879 | 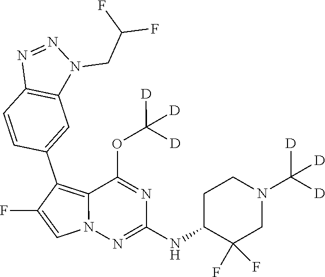 [SMILES: [2H]C([2H])([2H])Oc1nc(N[C@@H]2CCN(C([2H])([2H])[2H])CC2(F)F)nn2cc(F)c(-c3ccc4nnn(CC(F)F)c4c3)c12] |
| 880 | 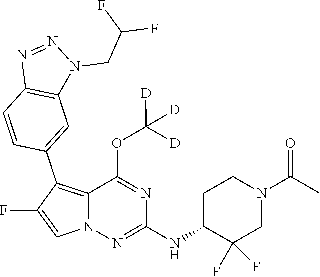 [SMILES: [2H]C([2H])([2H])Oc1nc(N[C@@H]2CCN(C(C)=O)CC2(F)F)nn2cc(F)c(-c3ccc4nnn(CC(F)F)c4c3)c12] |
| 881 | 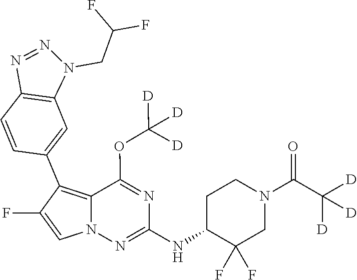 [SMILES: [2H]C([2H])([2H])Oc1nc(N[C@@H]2CCN(C(=O)C([2H])([2H])[2H])CC2(F)F)nn2cc(F)c(-c3ccc4nnn(CC(F)F)c4c3)c12] |
| 882 | 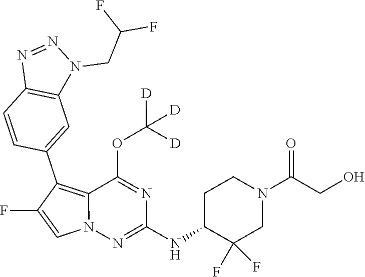 [SMILES: [2H]C([2H])([2H])Oc1nc(N[C@@H]2CCN(C(=O)CO)CC2(F)F)nn2cc(F)c(-c3ccc4nnn(CC(F)F)c4c3)c12] |
| 883 | 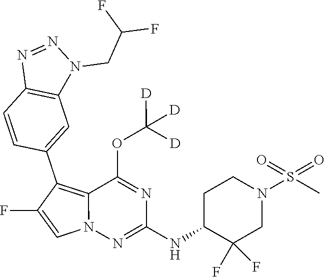 [SMILES: [2H]C([2H])([2H])Oc1nc(N[C@@H]2CCN(S(C)(=O)=O)CC2(F)F)nn2cc(F)c(-c3ccc4nnn(CC(F)F)c4c3)c12] |
| 884 | 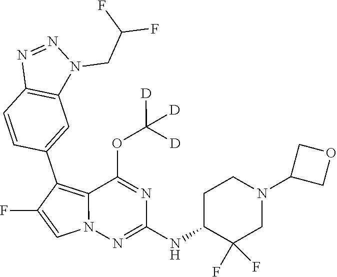 [SMILES: [2H]C([2H])([2H])Oc1nc(N[C@@H]2CCN(C3COC3)CC2(F)F)nn2cc(F)c(-c3ccc4nnn(CC(F)F)c4c3)c12] |
| 885 | 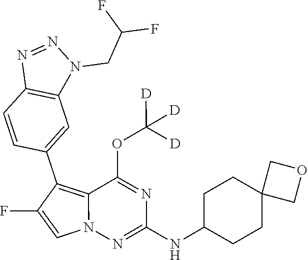 [SMILES: [2H]C([2H])([2H])Oc1nc(NC2CCC3(CC2)COC3)nn2cc(F)c(-c3ccc4nnn(CC(F)F)c4c3)c12] |
| 886 | 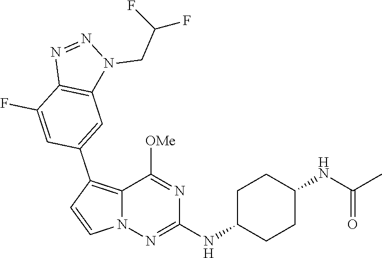 [SMILES: COc1nc(N[C@H]2CC[C@@H](NC(C)=O)CC2)nn2ccc(-c3cc(F)c4nnn(CC(F)F)c4c3)c12] |
| 887 | 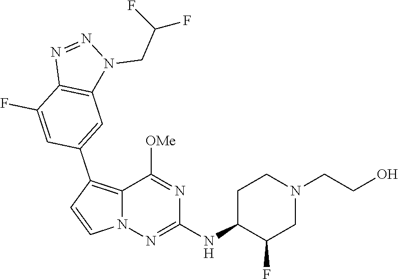 [SMILES: COc1nc(N[C@H]2CCN(CCO)C[C@H]2F)nn2ccc(-c3cc(F)c4nnn(CC(F)F)c4c3)c12] |
| 888 | 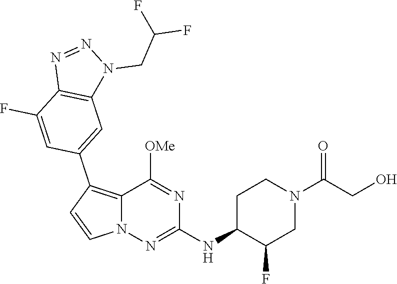 [SMILES: COc1nc(N[C@H]2CCN(C(=O)CO)C[C@H]2F)nn2ccc(-c3cc(F)c4nnn(CC(F)F)c4c3)c12] |
| 889 | 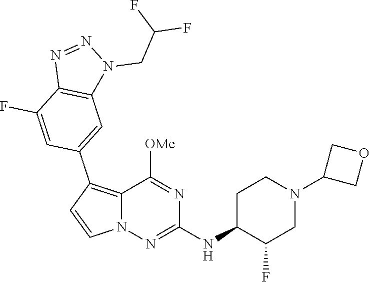 [SMILES: COc1nc(N[C@H]2CCN(C3COC3)C[C@@H]2F)nn2ccc(-c3cc(F)c4nnn(CC(F)F)c4c3)c12] |
| 890 | 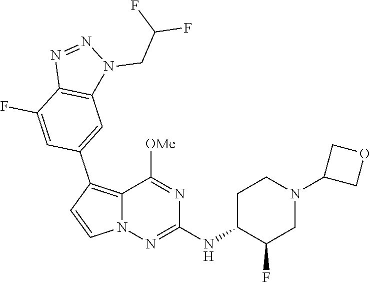 [SMILES: COc1nc(N[C@@H]2CCN(C3COC3)C[C@H]2F)nn2ccc(-c3cc(F)c4nnn(CC(F)F)c4c3)c12] |
| 891 | 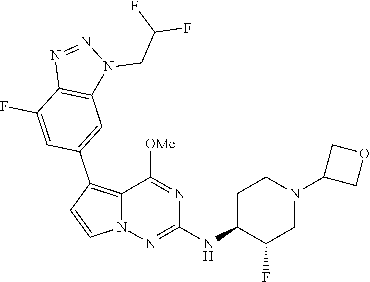 [SMILES: COc1nc(N[C@H]2CCN(C3COC3)C[C@@H]2F)nn2ccc(-c3cc(F)c4nnn(CC(F)F)c4c3)c12] |
| 892 | 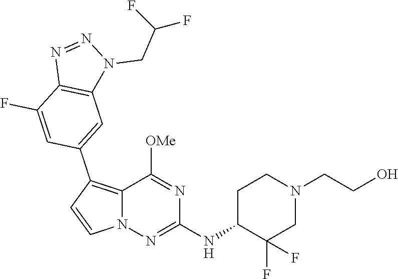 [SMILES: COc1nc(N[C@@H]2CCN(CCO)CC2(F)F)nn2ccc(-c3cc(F)c4nnn(CC(F)F)c4c3)c12] |
| 893 | 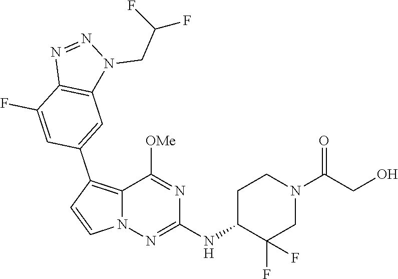 [SMILES: COc1nc(N[C@@H]2CCN(C(=O)CO)CC2(F)F)nn2ccc(-c3cc(F)c4nnn(CC(F)F)c4c3)c12] |
| 894 | 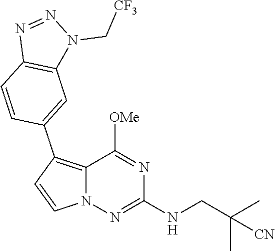 [SMILES: COc1nc(NCC(C)(C)C#N)nn2ccc(-c3ccc4nnn(CC(F)(F)F)c4c3)c12] |
| 895 | 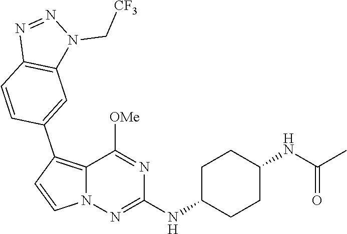 [SMILES: COc1nc(N[C@H]2CC[C@@H](NC(C)=O)CC2)nn2ccc(-c3ccc4nnn(CC(F)(F)F)c4c3)c12] |
| 896 | 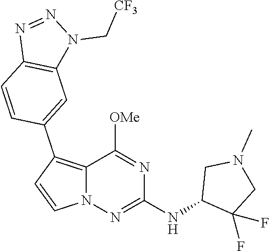 [SMILES: COc1nc(N[C@@H]2CN(C)CC2(F)F)nn2ccc(-c3ccc4nnn(CC(F)(F)F)c4c3)c12] |
| 897 | 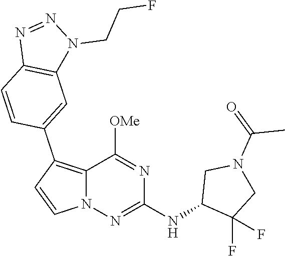 [SMILES: COc1nc(N[C@@H]2CN(C(C)=O)CC2(F)F)nn2ccc(-c3ccc4nnn(CCF)c4c3)c12] |
| 898 | 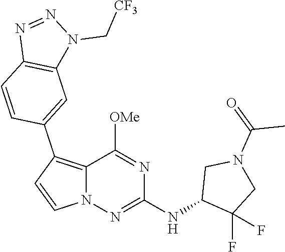 [SMILES: COc1nc(N[C@@H]2CN(C(C)=O)CC2(F)F)nn2ccc(-c3ccc4nnn(CC(F)(F)F)c4c3)c12] |
| 899 | 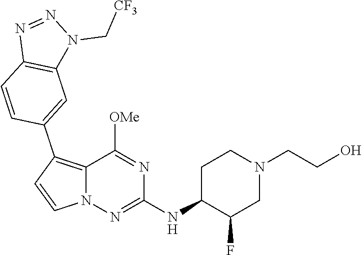 [SMILES: COc1nc(N[C@H]2CCN(CCO)C[C@H]2F)nn2ccc(-c3ccc4nnn(CC(F)(F)F)c4c3)c12] |
| 900 | 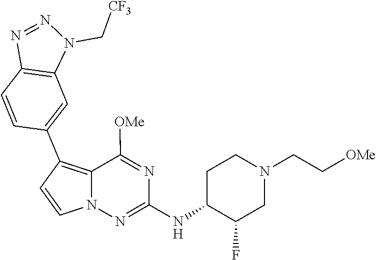 [SMILES: COCCN1CC[C@@H](Nc2nc(OC)c3c(-c4ccc5nnn(CC(F)(F)F)c5c4)ccn3n2)[C@@H](F)C1] |
| 901 | 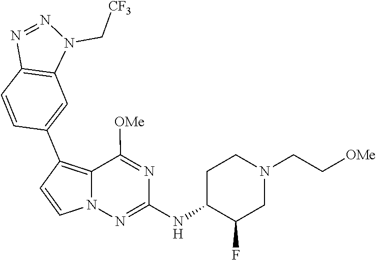 [SMILES: COCCN1CC[C@@H](Nc2nc(OC)c3c(-c4ccc5nnn(CC(F)(F)F)c5c4)ccn3n2)[C@H](F)C1] |
| 902 | 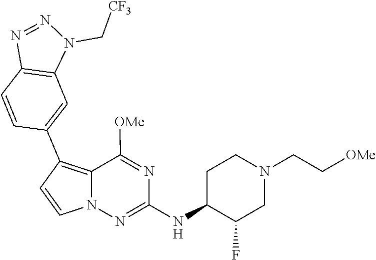 [SMILES: COCCN1CC[C@H](Nc2nc(OC)c3c(-c4ccc5nnn(CC(F)(F)F)c5c4)ccn3n2)[C@@H](F)C1] |
| 903 | 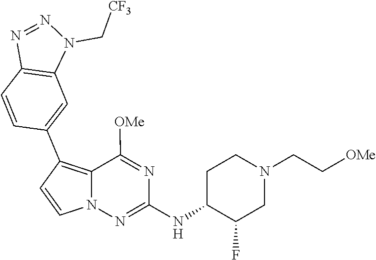 [SMILES: COCCN1CC[C@@H](Nc2nc(OC)c3c(-c4ccc5nnn(CC(F)(F)F)c5c4)ccn3n2)[C@@H](F)C1] |
| 904 | 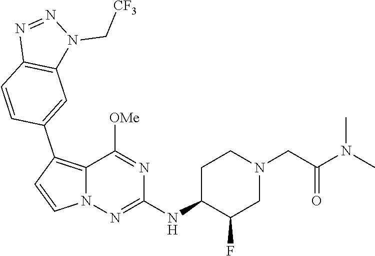 [SMILES: COc1nc(N[C@H]2CCN(CC(=O)N(C)C)C[C@H]2F)nn2ccc(-c3ccc4nnn(CC(F)(F)F)c4c3)c12] |
| 905 | 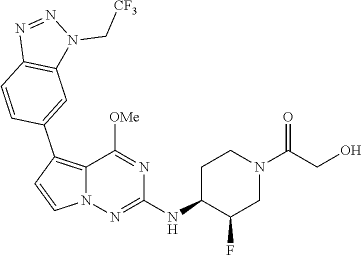 [SMILES: COc1nc(N[C@H]2CCN(C(=O)CO)C[C@H]2F)nn2ccc(-c3ccc4nnn(CC(F)(F)F)c4c3)c12] |
| 906 | 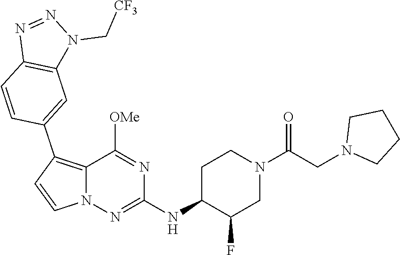 [SMILES: COc1nc(N[C@H]2CCN(C(=O)CN3CCCC3)C[C@H]2F)nn2ccc(-c3ccc4nnn(CC(F)(F)F)c4c3)c12] |
| 907 | 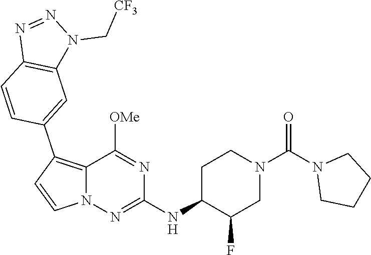 [SMILES: COc1nc(N[C@H]2CCN(C(=O)N3CCCC3)C[C@H]2F)nn2ccc(-c3ccc4nnn(CC(F)(F)F)c4c3)c12] |
| 908 | 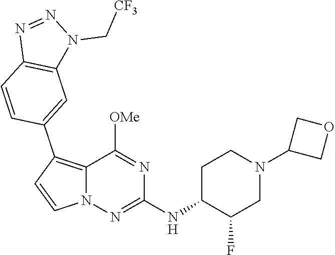 [SMILES: COc1nc(N[C@@H]2CCN(C3COC3)C[C@@H]2F)nn2ccc(-c3ccc4nnn(CC(F)(F)F)c4c3)c12] |
| 909 | 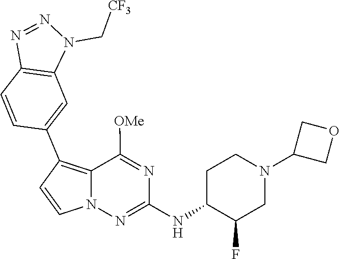 [SMILES: COc1nc(N[C@@H]2CCN(C3COC3)C[C@H]2F)nn2ccc(-c3ccc4nnn(CC(F)(F)F)c4c3)c12] |
| 910 | 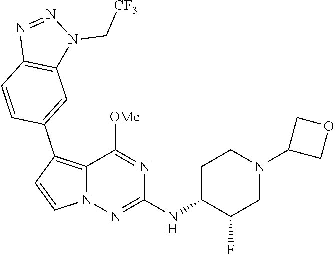 [SMILES: COc1nc(N[C@@H]2CCN(C3COC3)C[C@@H]2F)nn2ccc(-c3ccc4nnn(CC(F)(F)F)c4c3)c12] |
| 911 | 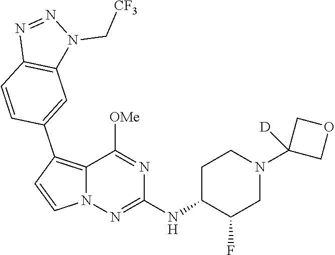 [SMILES: [2H]C1(N2CC[C@@H](Nc3nc(OC)c4c(-c5ccc6nnn(CC(F)(F)F)c6c5)ccn4n3)[C@@H](F)C2)COC1] |
| 912 | 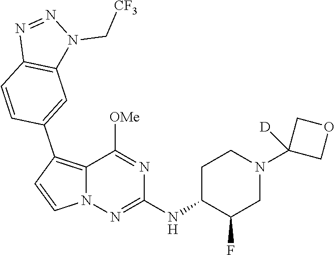 [SMILES: [2H]C1(N2CC[C@@H](Nc3nc(OC)c4c(-c5ccc6nnn(CC(F)(F)F)c6c5)ccn4n3)[C@H](F)C2)COC1] |
| 913 | 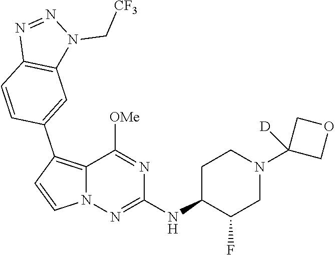 [SMILES: [2H]C1(N2CC[C@H](Nc3nc(OC)c4c(-c5ccc6nnn(CC(F)(F)F)c6c5)ccn4n3)[C@@H](F)C2)COC1] |
| 914 | 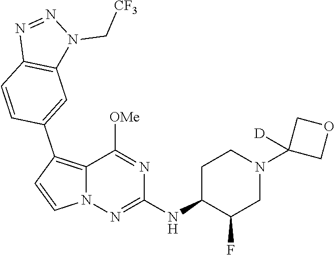 [SMILES: [2H]C1(N2CC[C@H](Nc3nc(OC)c4c(-c5ccc6nnn(CC(F)(F)F)c6c5)ccn4n3)[C@H](F)C2)COC1] |
| 915 | 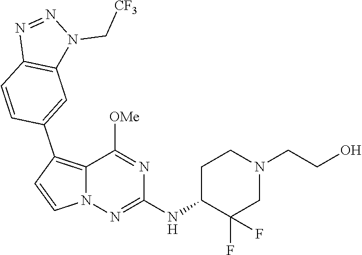 |
| 916 | 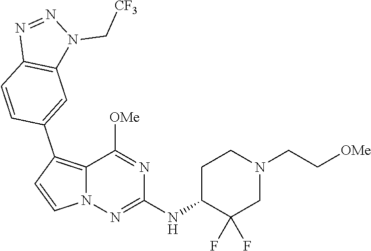 [SMILES: COCCN1CC[C@@H](Nc2nc(OC)c3c(-c4ccc5nnn(CC(F)(F)F)c5c4)ccn3n2)C(F)(F)C1] |
| 917 | 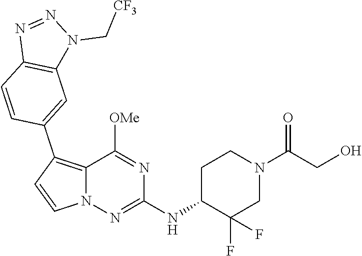 [SMILES: COc1nc(N[C@@H]2CCN(C(=O)CO)CC2(F)F)nn2ccc(-c3ccc4nnn(CC(F)(F)F)c4c3)c12] |
| 918 | 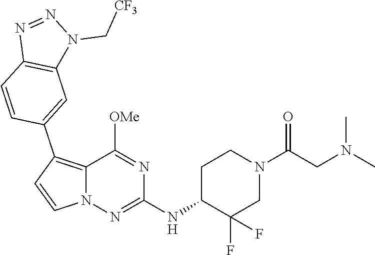 |
| 919 | 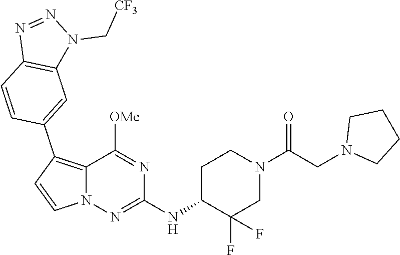 |
| 920 | 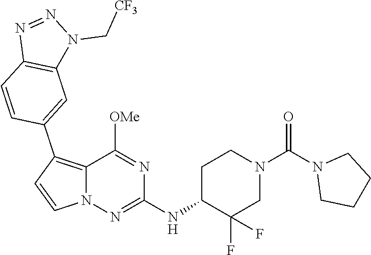 |
| 921 | 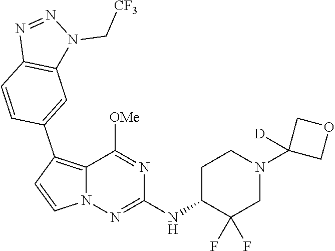 [SMILES: [2H]C1(N2CC[C@@H](Nc3nc(OC)c4c(-c5ccc6nnn(CC(F)(F)F)c6c5)ccn4n3)C(F)(F)C2)COC1] |
| 922 | 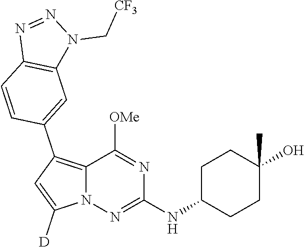 [SMILES: [2H]c1cc(-c2ccc3nnn(CC(F)(F)F)c3c2)c2c(OC)nc(N[C@H]3CC[C@@](C)(O)CC3)nn12] |
| 923 | 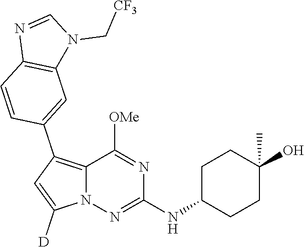 [SMILES: [2H]c1cc(-c2ccc3ncn(CC(F)(F)F)c3c2)c2c(OC)nc(N[C@H]3CC[C@](C)(O)CC3)nn12] |
| 924 | 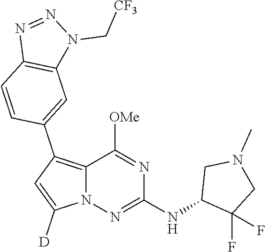 [SMILES: [2H]c1cc(-c2ccc3nnn(CC(F)(F)F)c3c2)c2c(OC)nc(N[C@@H]3CN(C)CC3(F)F)nn12] |
| 925 | 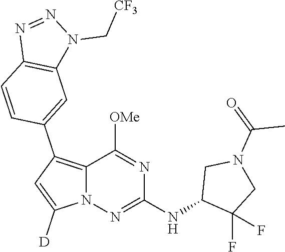 [SMILES: [2H]c1cc(-c2ccc3nnn(CC(F)(F)F)c3c2)c2c(OC)nc(N[C@@H]3CN(C(C)=O)CC3(F)F)nn12] |
| 926 | 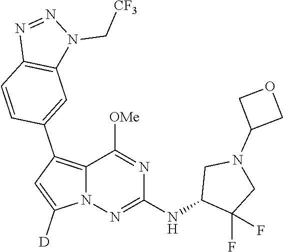 [SMILES: [2H]c1cc(-c2ccc3nnn(CC(F)(F)F)c3c2)c2c(OC)nc(N[C@@H]3CN(C4COC4)CC3(F)F)nn12] |
| 927 | 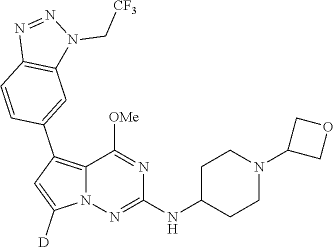 [SMILES: [2H]c1cc(-c2ccc3nnn(CC(F)(F)F)c3c2)c2c(OC)nc(NC3CCN(C4COC4)CC3)nn12] |
| 928 | 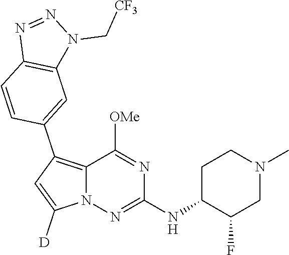 [SMILES: [2H]c1cc(-c2ccc3nnn(CC(F)(F)F)c3c2)c2c(OC)nc(N[C@@H]3CCN(C)C[C@@H]3F)nn12] |
| 929 | 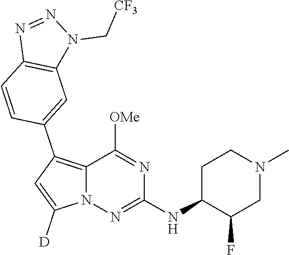 [SMILES: [2H]c1cc(-c2ccc3nnn(CC(F)(F)F)c3c2)c2c(OC)nc(N[C@H]3CCN(C)C[C@H]3F)nn12] |
| 930 | 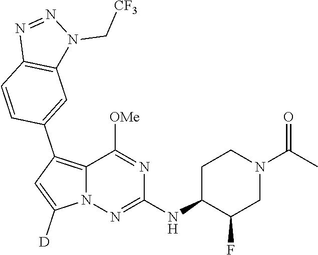 [SMILES: [2H]c1cc(-c2ccc3nnn(CC(F)(F)F)c3c2)c2c(OC)nc(N[C@H]3CCN(C(C)=O)C[C@H]3F)nn12] |
| 931 | 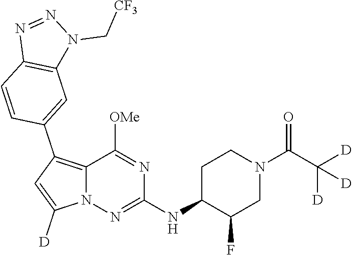 [SMILES: [2H]c1cc(-c2ccc3nnn(CC(F)(F)F)c3c2)c2c(OC)nc(N[C@H]3CCN(C(=O)C([2H])([2H])[2H])C[C@H]3F)nn12] |
| 932 | 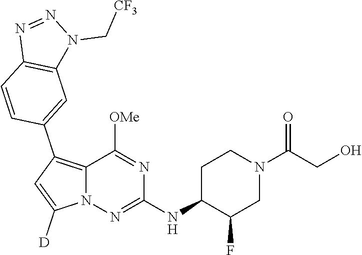 [SMILES: [2H]c1cc(-c2ccc3nnn(CC(F)(F)F)c3c2)c2c(OC)nc(N[C@H]3CCN(C(=O)CO)C[C@H]3F)nn12] |
| 933 | 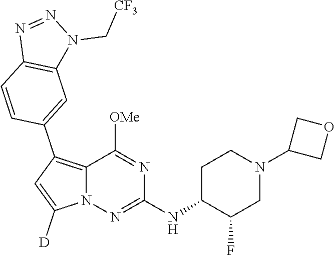 [SMILES: [2H]c1cc(-c2ccc3nnn(CC(F)(F)F)c3c2)c2c(OC)nc(N[C@@H]3CCN(C4COC4)C[C@@H]3F)nn12] |
| 934 | 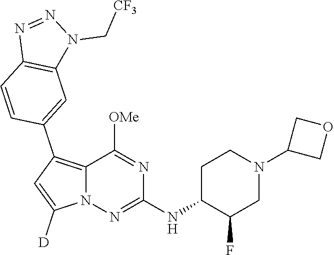 [SMILES: [2H]c1cc(-c2ccc3nnn(CC(F)(F)F)c3c2)c2c(OC)nc(N[C@@H]3CCN(C4COC4)C[C@H]3F)nn12] |
| 935 | 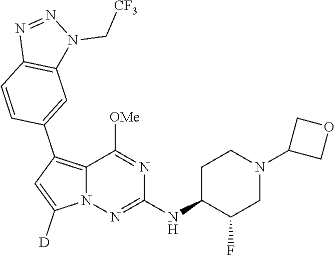 [SMILES: [2H]c1cc(-c2ccc3nnn(CC(F)(F)F)c3c2)c2c(OC)nc(N[C@H]3CCN(C4COC4)C[C@@H]3F)nn12] |
| 936 | 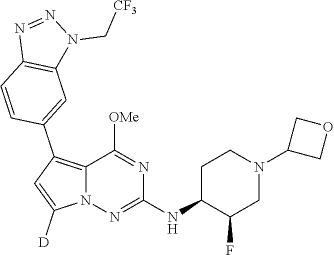 [SMILES: [2H]c1cc(-c2ccc3nnn(CC(F)(F)F)c3c2)c2c(OC)nc(N[C@H]3CCN(C4COC4)C[C@H]3F)nn12] |
| 937 | 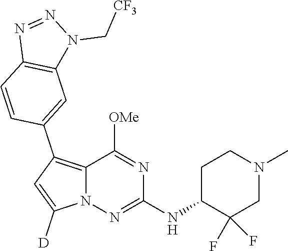 [SMILES: [2H]c1cc(-c2ccc3nnn(CC(F)(F)F)c3c2)c2c(OC)nc(N[C@@H]3CCN(C)CC3(F)F)nn12] |
| 938 | 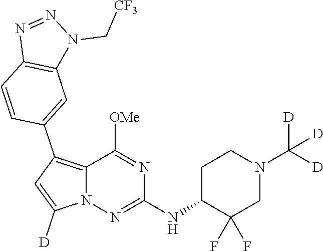 [SMILES: [2H]c1cc(-c2ccc3nnn(CC(F)(F)F)c3c2)c2c(OC)nc(N[C@@H]3CCN(C([2H])([2H])[2H])CC3(F)F)nn12] |
| 939 | 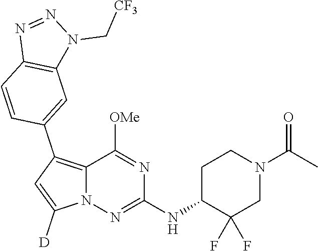 [SMILES: [2H]c1cc(-c2ccc3nnn(CC(F)(F)F)c3c2)c2c(OC)nc(N[C@@H]3CCN(C(C)=O)CC3(F)F)nn12] |
| 940 | 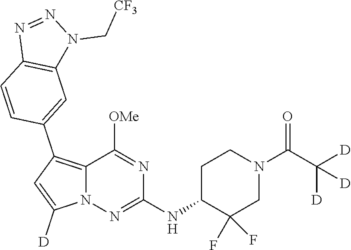 [SMILES: [2H]c1cc(-c2ccc3nnn(CC(F)(F)F)c3c2)c2c(OC)nc(N[C@@H]3CCN(C(=O)C([2H])([2H])[2H])CC3(F)F)nn12] |
| 941 | 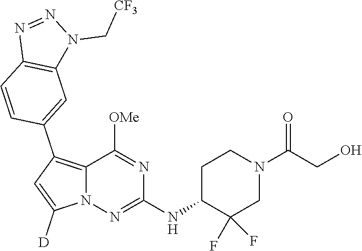 [SMILES: [2H]c1cc(-c2ccc3nnn(CC(F)(F)F)c3c2)c2c(OC)nc(N[C@@H]3CCN(C(=O)CO)CC3(F)F)nn12] |
| 942 | 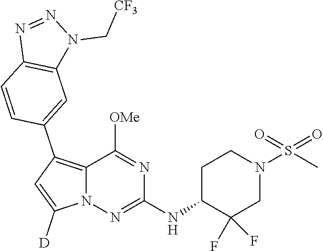 [SMILES: [2H]c1cc(-c2ccc3nnn(CC(F)(F)F)c3c2)c2c(OC)nc(N[C@@H]3CCN(S(C)(=O)=O)CC3(F)F)nn12] |
| 943 | 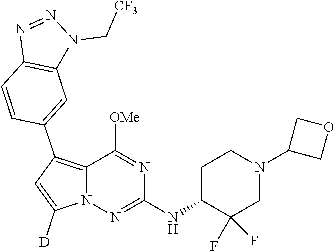 [SMILES: [2H]c1cc(-c2ccc3nnn(CC(F)(F)F)c3c2)c2c(OC)nc(N[C@@H]3CCN(C4COC4)CC3(F)F)nn12] |
| 944 | 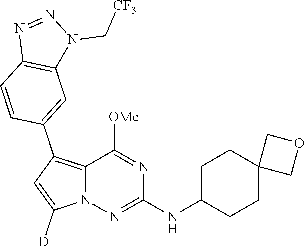 [SMILES: [2H]c1cc(-c2ccc3nnn(CC(F)(F)F)c3c2)c2c(OC)nc(NC3CCC4(CC3)COC4)nn12] |
| 945 | 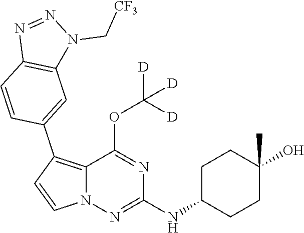 [SMILES: [2H]C([2H])([2H])Oc1nc(N[C@H]2CC[C@@](C)(O)CC2)nn2ccc(-c3ccc4nnn(CC(F)(F)F)c4c3)c12] |
| 946 | 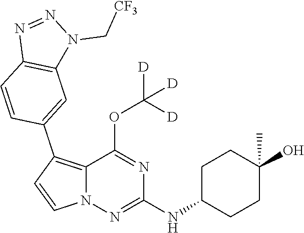 [SMILES: [2H]C([2H])([2H])Oc1nc(N[C@H]2CC[C@](C)(O)CC2)nn2ccc(-c3ccc4nnn(CC(F)(F)F)c4c3)c12] |
| 947 | 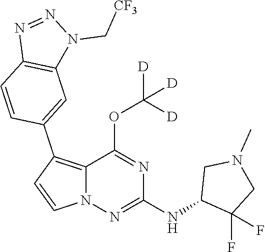 [SMILES: [2H]C([2H])([2H])Oc1nc(N[C@@H]2CN(C)CC2(F)F)nn2ccc(-c3ccc4nnn(CC(F)(F)F)c4c3)c12] |
| 948 | 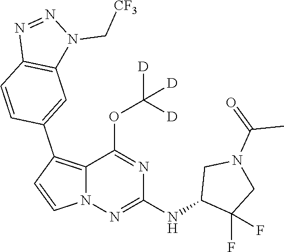 [SMILES: [2H]C([2H])([2H])Oc1nc(N[C@@H]2CN(C(C)=O)CC2(F)F)nn2ccc(-c3ccc4nnn(CC(F)(F)F)c4c3)c12] |
| 949 | 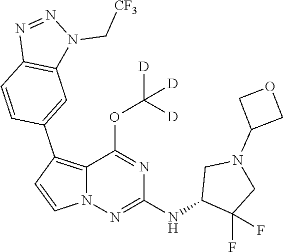 [SMILES: [2H]C([2H])([2H])Oc1nc(N[C@@H]2CN(C3COC3)CC2(F)F)nn2ccc(-c3ccc4nnn(CC(F)(F)F)c4c3)c12] |
| 950 | 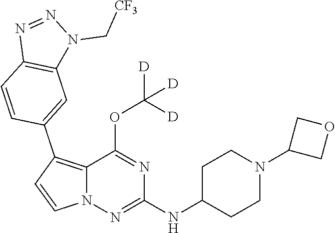 [SMILES: [2H]C([2H])([2H])Oc1nc(NC2CCN(C3COC3)CC2)nn2ccc(-c3ccc4nnn(CC(F)(F)F)c4c3)c12] |
| 951 | 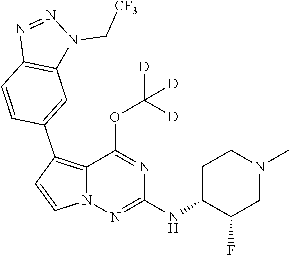 [SMILES: [2H]C([2H])([2H])Oc1nc(N[C@@H]2CCN(C)C[C@@H]2F)nn2ccc(-c3ccc4nnn(CC(F)(F)F)c4c3)c12] |
| 952 | 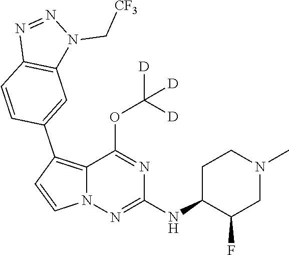 [SMILES: [2H]C([2H])([2H])Oc1nc(N[C@H]2CCN(C)C[C@H]2F)nn2ccc(-c3ccc4nnn(CC(F)(F)F)c4c3)c12] |
| 953 | 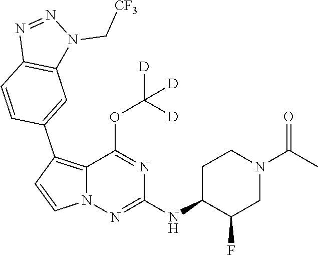 [SMILES: [2H]C([2H])([2H])Oc1nc(N[C@H]2CCN(C(C)=O)C[C@H]2F)nn2ccc(-c3ccc4nnn(CC(F)(F)F)c4c3)c12] |
| 954 | 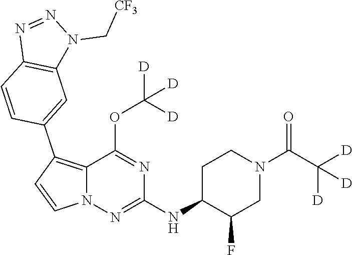 [SMILES: [2H]C([2H])([2H])Oc1nc(N[C@H]2CCN(C(=O)C([2H])([2H])[2H])C[C@H]2F)nn2ccc(-c3ccc4nnn(CC(F)(F)F)c4c3)c12] |
| 955 | 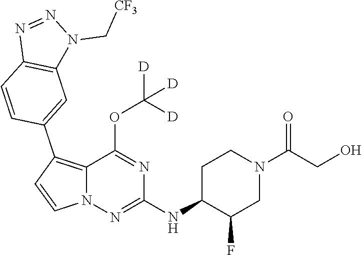 [SMILES: [2H]C([2H])([2H])Oc1nc(N[C@H]2CCN(C(=O)CO)C[C@H]2F)nn2ccc(-c3ccc4nnn(CC(F)(F)F)c4c3)c12] |
| 956 | 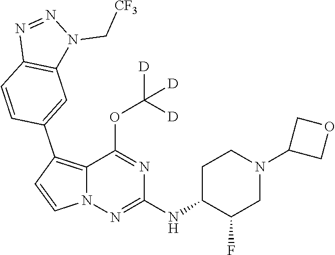 [SMILES: [2H]C([2H])([2H])Oc1nc(N[C@@H]2CCN(C3COC3)C[C@@H]2F)nn2ccc(-c3ccc4nnn(CC(F)(F)F)c4c3)c12] |
| 957 | 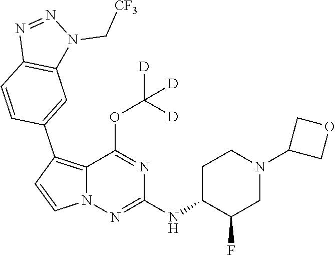 [SMILES: [2H]C([2H])([2H])Oc1nc(N[C@@H]2CCN(C3COC3)C[C@H]2F)nn2ccc(-c3ccc4nnn(CC(F)(F)F)c4c3)c12] |
| 958 | 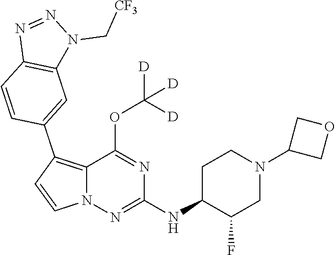 [SMILES: [2H]C([2H])([2H])Oc1nc(N[C@H]2CCN(C3COC3)C[C@@H]2F)nn2ccc(-c3ccc4nnn(CC(F)(F)F)c4c3)c12] |
| 959 | 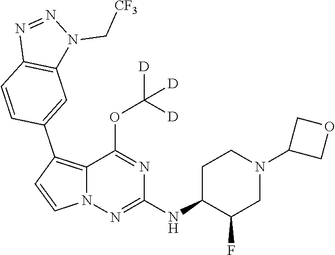 [SMILES: [2H]C([2H])([2H])Oc1nc(N[C@H]2CCN(C3COC3)C[C@H]2F)nn2ccc(-c3ccc4nnn(CC(F)(F)F)c4c3)c12] |
| 960 | 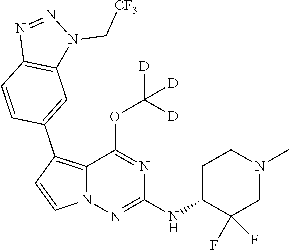 [SMILES: [2H]C([2H])([2H])Oc1nc(N[C@@H]2CCN(C)CC2(F)F)nn2ccc(-c3ccc4nnn(CC(F)(F)F)c4c3)c12] |
| 961 | 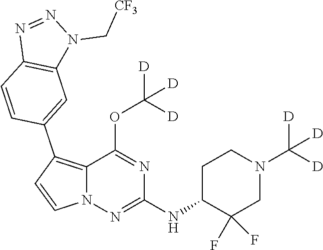 [SMILES: [2H]C([2H])([2H])Oc1nc(N[C@@H]2CCN(C([2H])([2H])[2H])CC2(F)F)nn2ccc(-c3ccc4nnn(CC(F)(F)F)c4c3)c12] |
| 962 | 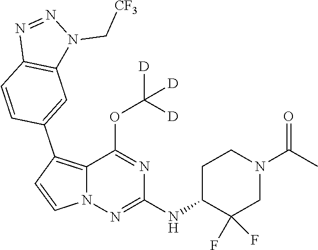 [SMILES: [2H]C([2H])([2H])Oc1nc(N[C@@H]2CCN(C(C)=O)CC2(F)F)nn2ccc(-c3ccc4nnn(CC(F)(F)F)c4c3)c12] |
| 963 | 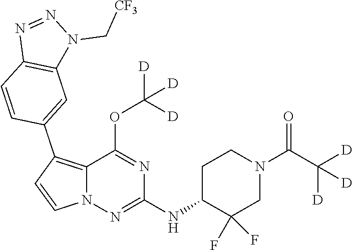 [SMILES: [2H]C([2H])([2H])Oc1nc(N[C@@H]2CCN(C(=O)C([2H])([2H])[2H])CC2(F)F)nn2ccc(-c3ccc4nnn(CC(F)(F)F)c4c3)c12] |
| 964 | 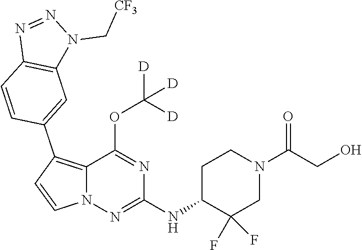 [SMILES: [2H]C([2H])([2H])Oc1nc(N[C@@H]2CCN(C(=O)CO)CC2(F)F)nn2ccc(-c3ccc4nnn(CC(F)(F)F)c4c3)c12] |
| 965 | 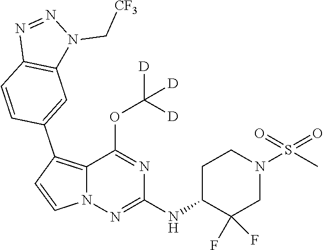 [SMILES: [2H]C([2H])([2H])Oc1nc(N[C@@H]2CCN(S(C)(=O)=O)CC2(F)F)nn2ccc(-c3ccc4nnn(CC(F)(F)F)c4c3)c12] |
| 966 | 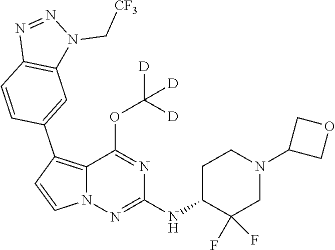 [SMILES: [2H]C([2H])([2H])Oc1nc(N[C@@H]2CCN(C3COC3)CC2(F)F)nn2ccc(-c3ccc4nnn(CC(F)(F)F)c4c3)c12] |
| 967 | 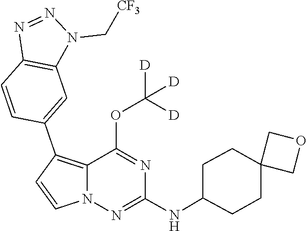 [SMILES: [2H]C([2H])([2H])Oc1nc(NC2CCC3(CC2)COC3)nn2ccc(-c3ccc4nnn(CC(F)(F)F)c4c3)c12] |
| 968 | 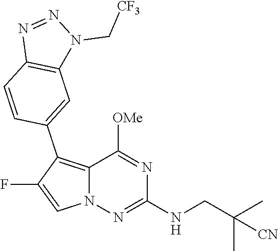 [SMILES: COc1nc(NCC(C)(C)C#N)nn2cc(F)c(-c3ccc4nnn(CC(F)(F)F)c4c3)c12] |
| 969 | 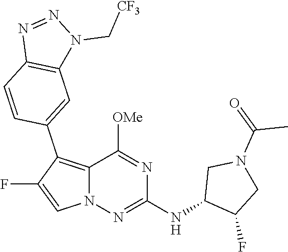 [SMILES: COc1nc(N[C@@H]2CN(C(C)=O)C[C@@H]2F)nn2cc(F)c(-c3ccc4nnn(CC(F)(F)F)c4c3)c12] |
| 970 | 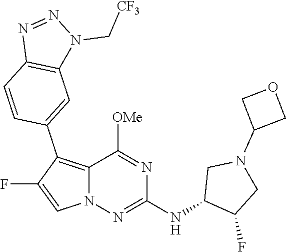 [SMILES: COc1nc(N[C@@H]2CN(C3COC3)C[C@@H]2F)nn2cc(F)c(-c3ccc4nnn(CC(F)(F)F)c4c3)c12] |
| 971 | 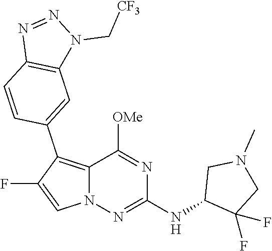 [SMILES: COc1nc(N[C@@H]2CN(C)CC2(F)F)nn2cc(F)c(-c3ccc4nnn(CC(F)(F)F)c4c3)c12] |
| 972 | 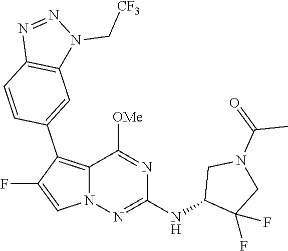 [SMILES: COc1nc(N[C@@H]2CN(C(C)=O)CC2(F)F)nn2cc(F)c(-c3ccc4nnn(CC(F)(F)F)c4c3)c12] |
| 973 | 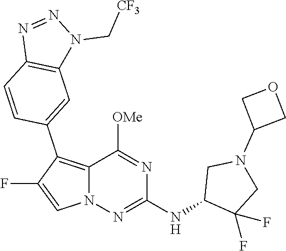 [SMILES: COc1nc(N[C@@H]2CN(C3COC3)CC2(F)F)nn2cc(F)c(-c3ccc4nnn(CC(F)(F)F)c4c3)c12] |
| 974 | 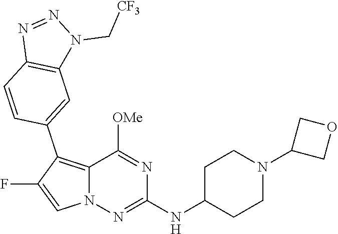 [SMILES: COc1nc(NC2CCN(C3COC3)CC2)nn2cc(F)c(-c3ccc4nnn(CC(F)(F)F)c4c3)c12] |
| 975 | 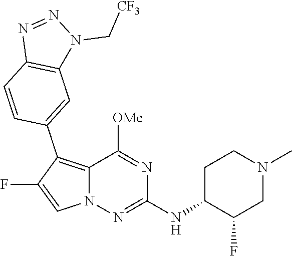 [SMILES: COc1nc(N[C@@H]2CCN(C)C[C@@H]2F)nn2cc(F)c(-c3ccc4nnn(CC(F)(F)F)c4c3)c12] |
| 976 | 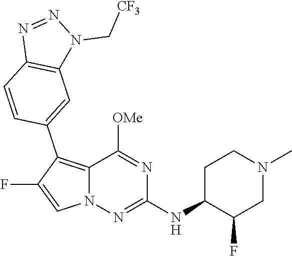 [SMILES: COc1nc(N[C@H]2CCN(C)C[C@H]2F)nn2cc(F)c(-c3ccc4nnn(CC(F)(F)F)c4c3)c12] |
| 977 | 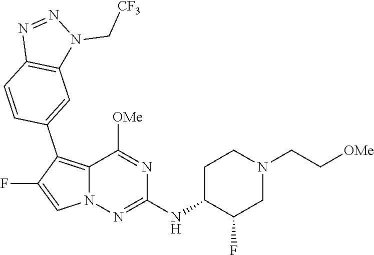 [SMILES: COCCN1CC[C@@H](Nc2nc(OC)c3c(-c4ccc5nnn(CC(F)(F)F)c5c4)c(F)cn3n2)[C@@H](F)C1] |
| 978 | 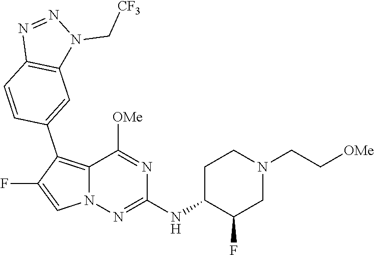 [SMILES: COCCN1CC[C@@H](Nc2nc(OC)c3c(-c4ccc5nnn(CC(F)(F)F)c5c4)c(F)cn3n2)[C@H](F)C1] |
| 979 | 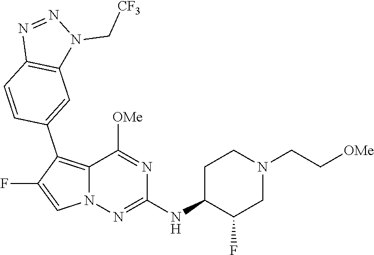 [SMILES: COCCN1CC[C@H](Nc2nc(OC)c3c(-c4ccc5nnn(CC(F)(F)F)c5c4)c(F)cn3n2)[C@@H](F)C1] |
| 980 | 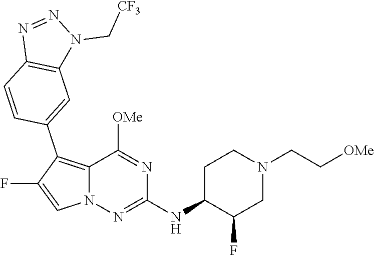 [SMILES: COCCN1CC[C@H](Nc2nc(OC)c3c(-c4ccc5nnn(CC(F)(F)F)c5c4)c(F)cn3n2)[C@H](F)C1] |
| 981 | 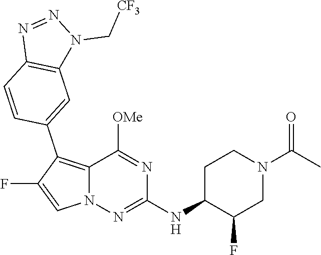 [SMILES: COc1nc(N[C@H]2CCN(C(C)=O)C[C@H]2F)nn2cc(F)c(-c3ccc4nnn(CC(F)(F)F)c4c3)c12] |
| 982 | 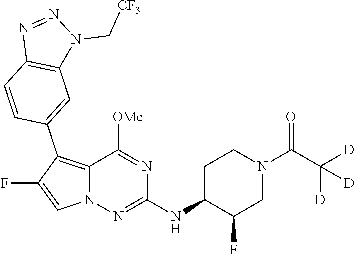 [SMILES: [2H]C([2H])([2H])C(=O)N1CC[C@H](Nc2nc(OC)c3c(-c4ccc5nnn(CC(F)(F)F)c5c4)c(F)cn3n2)[C@H](F)C1] |
| 983 | 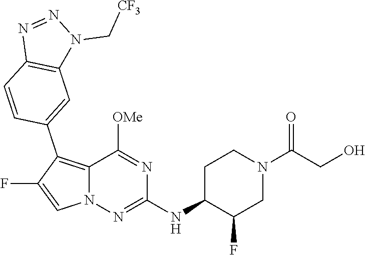 [SMILES: COc1nc(N[C@H]2CCN(C(=O)CO)C[C@H]2F)nn2cc(F)c(-c3ccc4nnn(CC(F)(F)F)c4c3)c12] |
| 984 | 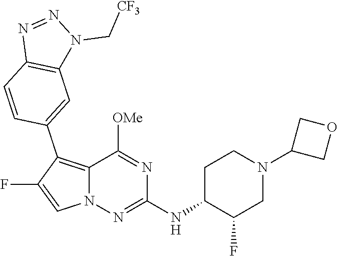 [SMILES: COc1nc(N[C@@H]2CCN(C3COC3)C[C@@H]2F)nn2cc(F)c(-c3ccc4nnn(CC(F)(F)F)c4c3)c12] |
| 985 | 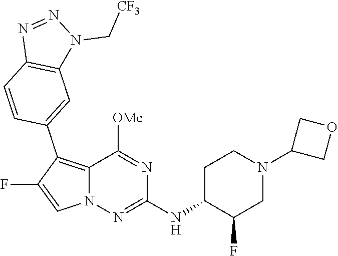 [SMILES: COc1nc(N[C@@H]2CCN(C3COC3)C[C@H]2F)nn2cc(F)c(-c3ccc4nnn(CC(F)(F)F)c4c3)c12] |
| 986 | 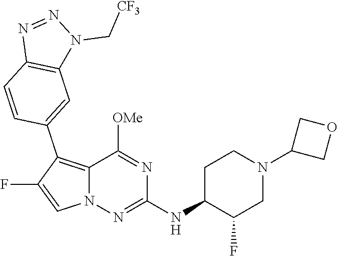 [SMILES: COc1nc(N[C@H]2CCN(C3COC3)C[C@@H]2F)nn2cc(F)c(-c3ccc4nnn(CC(F)(F)F)c4c3)c12] |
| 987 | 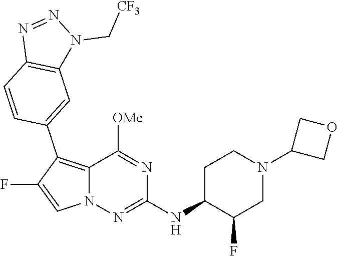 [SMILES: COc1nc(N[C@H]2CCN(C3COC3)C[C@H]2F)nn2cc(F)c(-c3ccc4nnn(CC(F)(F)F)c4c3)c12] |
| 988 | 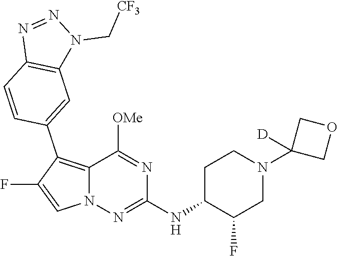 [SMILES: [2H]C1(N2CC[C@@H](Nc3nc(OC)c4c(-c5ccc6nnn(CC(F)(F)F)c6c5)c(F)cn4n3)[C@@H](F)C2)COC1] |
| 989 | 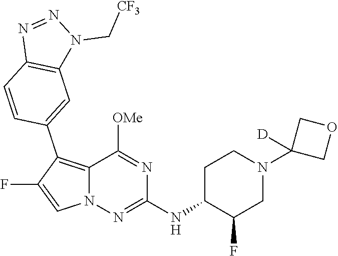 [SMILES: [2H]C1(N2CC[C@@H](Nc3nc(OC)c4c(-c5ccc6nnn(CC(F)(F)F)c6c5)c(F)cn4n3)[C@H](F)C2)COC1] |
| 990 | 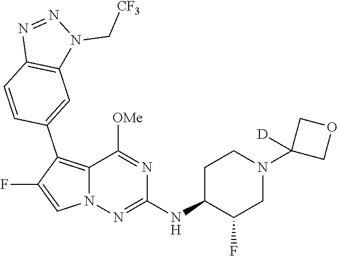 [SMILES: [2H]C1(N2CC[C@H](Nc3nc(OC)c4c(-c5ccc6nnn(CC(F)(F)F)c6c5)c(F)cn4n3)[C@@H](F)C2)COC1] |
| 991 | 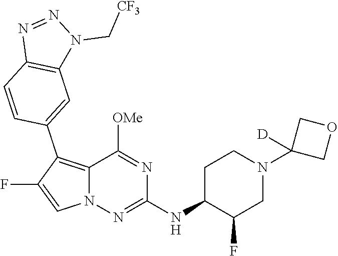 [SMILES: [2H]C1(N2CC[C@H](Nc3nc(OC)c4c(-c5ccc6nnn(CC(F)(F)F)c6c5)c(F)cn4n3)[C@H](F)C2)COC1] |
| 992 | 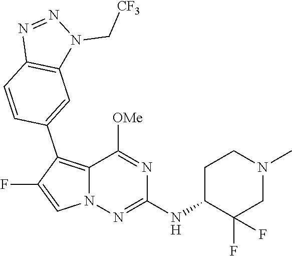 [SMILES: COc1nc(N[C@@H]2CCN(C)CC2(F)F)nn2cc(F)c(-c3ccc4nnn(CC(F)(F)F)c4c3)c12] |
| 993 | 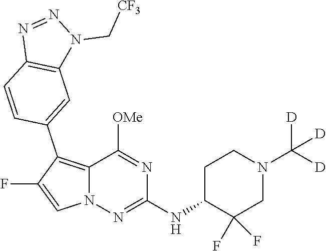 [SMILES: [2H]C([2H])([2H])N1CC[C@@H](Nc2nc(OC)c3c(-c4ccc5nnn(CC(F)(F)F)c5c4)c(F)cn3n2)C(F)(F)C1] |
| 994 | 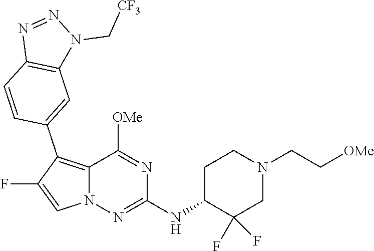 [SMILES: COCCN1CC[C@@H](Nc2nc(OC)c3c(-c4ccc5nnn(CC(F)(F)F)c5c4)c(F)cn3n2)C(F)(F)C1] |
| 995 | 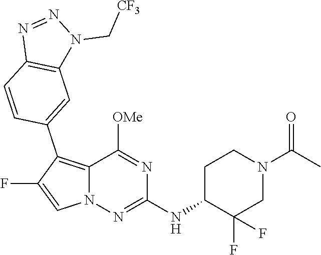 [SMILES: COc1nc(N[C@@H]2CCN(C(C)=O)CC2(F)F)nn2cc(F)c(-c3ccc4nnn(CC(F)(F)F)c4c3)c12] |
| 996 | 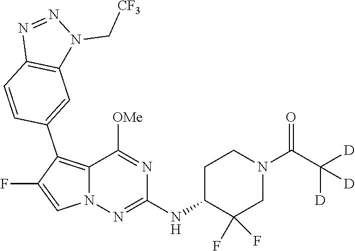 [SMILES: [2H]C([2H])([2H])C(=O)N1CC[C@@H](Nc2nc(OC)c3c(-c4ccc5nnn(CC(F)(F)F)c5c4)c(F)cn3n2)C(F)(F)C1] |
| 997 | 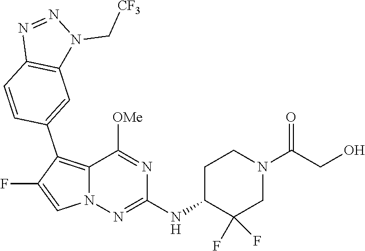 [SMILES: COc1nc(N[C@@H]2CCN(C(=O)CO)CC2(F)F)nn2cc(F)c(-c3ccc4nnn(CC(F)(F)F)c4c3)c12] |
| 998 | 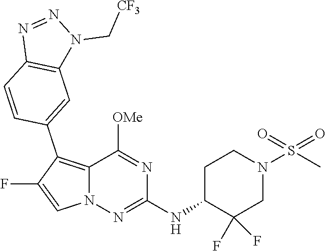 [SMILES: COc1nc(N[C@@H]2CCN(S(C)(=O)=O)CC2(F)F)nn2cc(F)c(-c3ccc4nnn(CC(F)(F)F)c4c3)c12] |
| 999 | 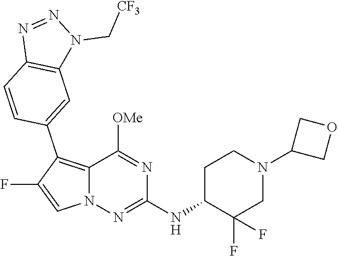 [SMILES: COc1nc(N[C@@H]2CCN(C3COC3)CC2(F)F)nn2cc(F)c(-c3ccc4nnn(CC(F)(F)F)c4c3)c12] |
| 1000 | 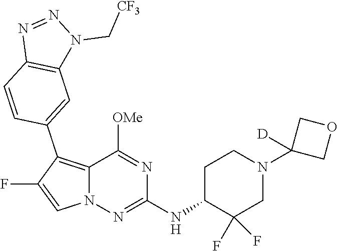 [SMILES: [2H]C1(N2CC[C@@H](Nc3nc(OC)c4c(-c5ccc6nnn(CC(F)(F)F)c6c5)c(F)cn4n3)C(F)(F)C2)COC1] |
| 1001 | 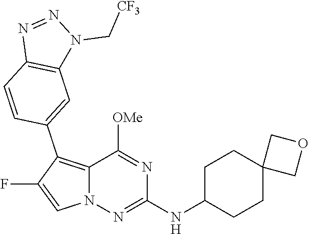 [SMILES: COc1nc(NC2CCC3(CC2)COC3)nn2cc(F)c(-c3ccc4nnn(CC(F)(F)F)c4c3)c12] |
| 1002 | 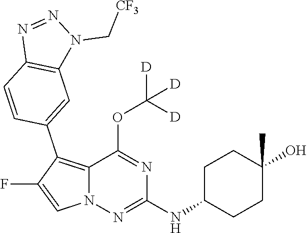 [SMILES: [2H]C([2H])([2H])Oc1nc(N[C@H]2CC[C@@](C)(O)CC2)nn2cc(F)c(-c3ccc4nnn(CC(F)(F)F)c4c3)c12] |
| 1003 | 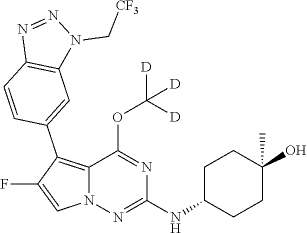 [SMILES: [2H]C([2H])([2H])Oc1nc(N[C@H]2CC[C@](C)(O)CC2)nn2cc(F)c(-c3ccc4nnn(CC(F)(F)F)c4c3)c12] |
| 1004 | 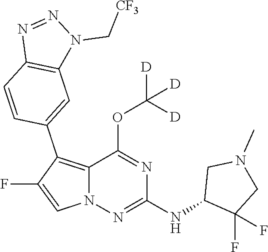 [SMILES: [2H]C([2H])([2H])Oc1nc(N[C@@H]2CN(C)CC2(F)F)nn2cc(F)c(-c3ccc4nnn(CC(F)(F)F)c4c3)c12] |
| 1005 | 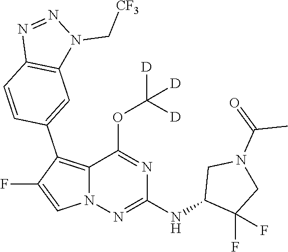 [SMILES: [2H]C([2H])([2H])Oc1nc(N[C@@H]2CN(C(C)=O)CC2(F)F)nn2cc(F)c(-c3ccc4nnn(CC(F)(F)F)c4c3)c12] |
| 1006 | 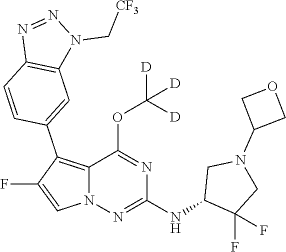 [SMILES: [2H]C([2H])([2H])Oc1nc(N[C@@H]2CN(C3COC3)CC2(F)F)nn2cc(F)c(-c3ccc4nnn(CC(F)(F)F)c4c3)c12] |
| 1007 | 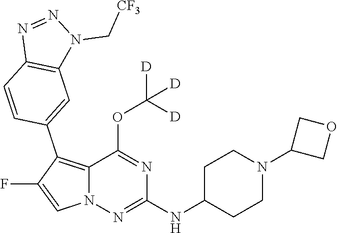 [SMILES: [2H]C([2H])([2H])Oc1nc(NC2CCN(C3COC3)CC2)nn2cc(F)c(-c3ccc4nnn(CC(F)(F)F)c4c3)c12] |
| 1008 | 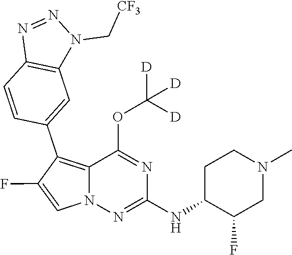 [SMILES: [2H]C([2H])([2H])Oc1nc(N[C@@H]2CCN(C)C[C@@H]2F)nn2cc(F)c(-c3ccc4nnn(CC(F)(F)F)c4c3)c12] |
| 1009 | 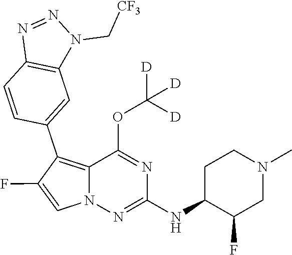 [SMILES: [2H]C([2H])([2H])Oc1nc(N[C@H]2CCN(C)C[C@H]2F)nn2cc(F)c(-c3ccc4nnn(CC(F)(F)F)c4c3)c12] |
| 1010 | 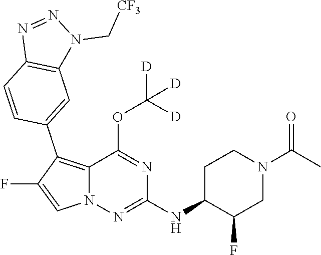 [SMILES: [2H]C([2H])([2H])Oc1nc(N[C@H]2CCN(C(C)=O)C[C@H]2F)nn2cc(F)c(-c3ccc4nnn(CC(F)(F)F)c4c3)c12] |
| 1011 | 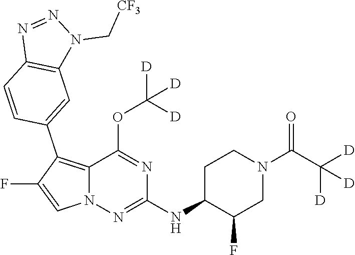 [SMILES: [2H]C([2H])([2H])Oc1nc(N[C@H]2CCN(C(=O)C([2H])([2H])[2H])C[C@H]2F)nn2cc(F)c(-c3ccc4nnn(CC(F)(F)F)c4c3)c12] |
| 1012 | 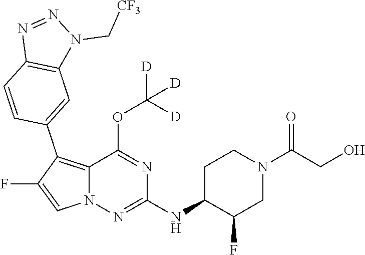 [SMILES: [2H]C([2H])([2H])Oc1nc(N[C@H]2CCN(C(=O)CO)C[C@H]2F)nn2cc(F)c(-c3ccc4nnn(CC(F)(F)F)c4c3)c12] |
| 1013 | 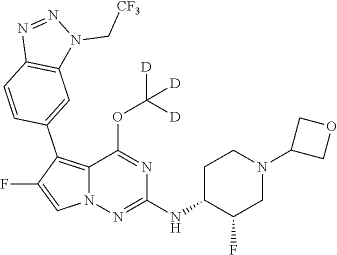 [SMILES: [2H]C([2H])([2H])Oc1nc(N[C@@H]2CCN(C3COC3)C[C@@H]2F)nn2cc(F)c(-c3ccc4nnn(CC(F)(F)F)c4c3)c12] |
| 1014 | 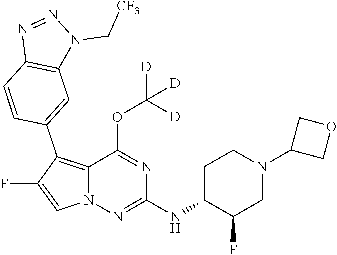 [SMILES: [2H]C([2H])([2H])Oc1nc(N[C@@H]2CCN(C3COC3)C[C@H]2F)nn2cc(F)c(-c3ccc4nnn(CC(F)(F)F)c4c3)c12] |
| 1015 | 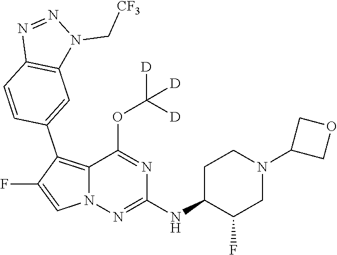 [SMILES: [2H]C([2H])([2H])Oc1nc(N[C@H]2CCN(C3COC3)C[C@@H]2F)nn2cc(F)c(-c3ccc4nnn(CC(F)(F)F)c4c3)c12] |
| 1016 | 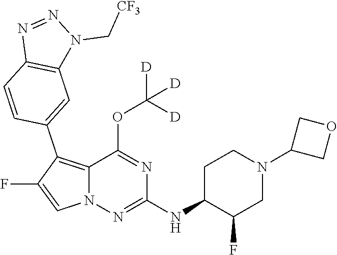 [SMILES: [2H]C([2H])([2H])Oc1nc(N[C@H]2CCN(C3COC3)C[C@H]2F)nn2cc(F)c(-c3ccc4nnn(CC(F)(F)F)c4c3)c12] |
| 1017 | 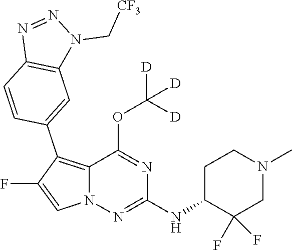 [SMILES: [2H]C([2H])([2H])Oc1nc(N[C@@H]2CCN(C)CC2(F)F)nn2cc(F)c(-c3ccc4nnn(CC(F)(F)F)c4c3)c12] |
| 1018 | 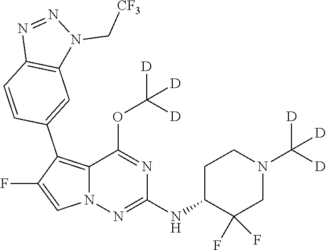 [SMILES: [2H]C([2H])([2H])Oc1nc(N[C@@H]2CCN(C([2H])([2H])[2H])CC2(F)F)nn2cc(F)c(-c3ccc4nnn(CC(F)(F)F)c4c3)c12] |
| 1019 | 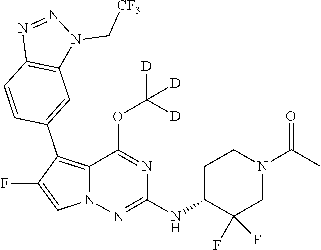 [SMILES: [2H]C([2H])([2H])Oc1nc(N[C@@H]2CCN(C(C)=O)CC2(F)F)nn2cc(F)c(-c3ccc4nnn(CC(F)(F)F)c4c3)c12] |
| 1020 | 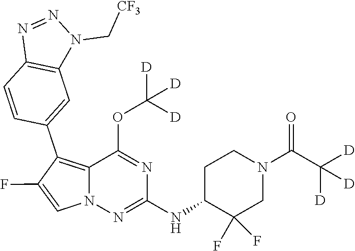 [SMILES: [2H]C([2H])([2H])Oc1nc(N[C@@H]2CCN(C(=O)C([2H])([2H])[2H])CC2(F)F)nn2cc(F)c(-c3ccc4nnn(CC(F)(F)F)c4c3)c12] |
| 1021 | 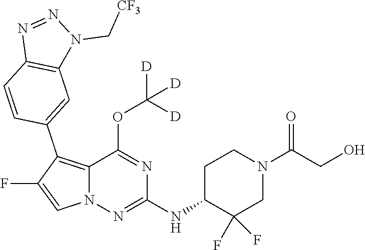 [SMILES: [2H]C([2H])([2H])Oc1nc(N[C@@H]2CCN(C(=O)CO)CC2(F)F)nn2cc(F)c(-c3ccc4nnn(CC(F)(F)F)c4c3)c12] |
| 1022 | 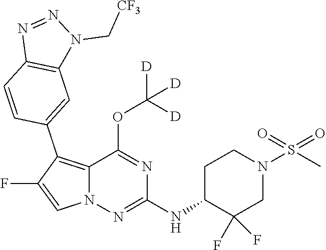 [SMILES: [2H]C([2H])([2H])Oc1nc(N[C@@H]2CCN(S(C)(=O)=O)CC2(F)F)nn2cc(F)c(-c3ccc4nnn(CC(F)(F)F)c4c3)c12] |
| 1023 | 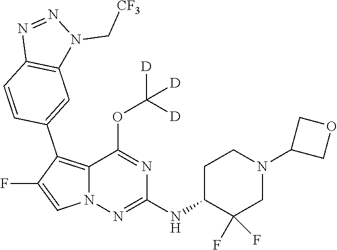 [SMILES: [2H]C([2H])([2H])Oc1nc(N[C@@H]2CCN(C3COC3)CC2(F)F)nn2cc(F)c(-c3ccc4nnn(CC(F)(F)F)c4c3)c12] |
| 1024 | 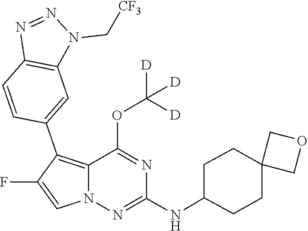 [SMILES: [2H]C([2H])([2H])Oc1nc(NC2CCC3(CC2)COC3)nn2cc(F)c(-c3ccc4nnn(CC(F)(F)F)c4c3)c12] |
| 1025 | 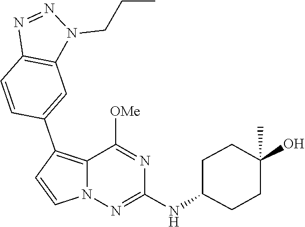 [SMILES: CCCn1nnc2ccc(-c3ccn4nc(N[C@H]5CC[C@](C)(O)CC5)nc(OC)c34)cc21] |
| 1026 | 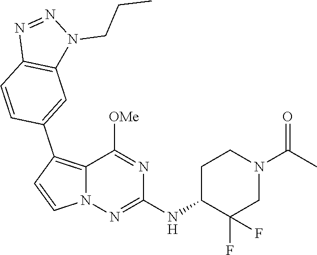 [SMILES: CCCn1nnc2ccc(-c3ccn4nc(N[C@@H]5CCN(C(C)=O)CC5(F)F)nc(OC)c34)cc21] |
| 1027 | 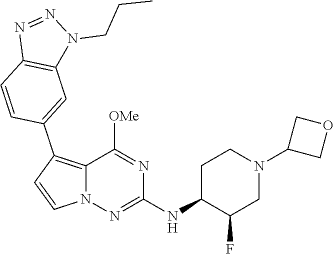 [SMILES: CCCn1nnc2ccc(-c3ccn4nc(N[C@H]5CCN(C6COC6)C[C@H]5F)nc(OC)c34)cc21] |
| 1028 | 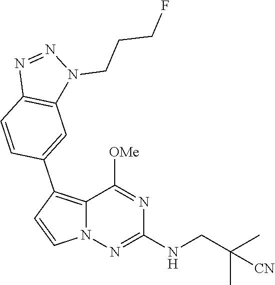 [SMILES: COc1nc(NCC(C)(C)C#N)nn2ccc(-c3ccc4nnn(CCCF)c4c3)c12] |
| 1029 | 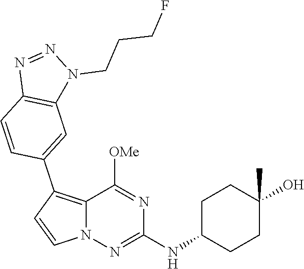 |
| 1030 | 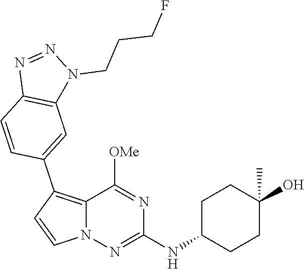 |
| 1031 | 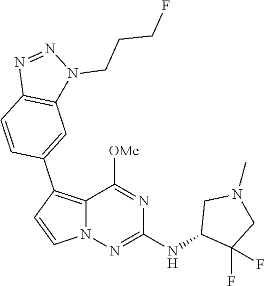 |
| 1032 | 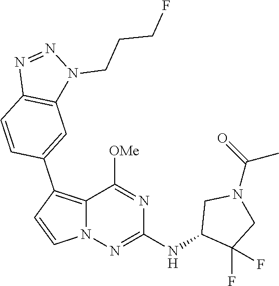 |
| 1033 | 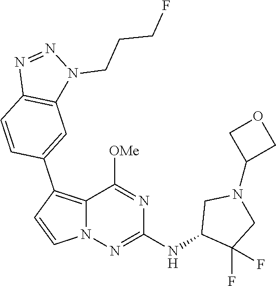 |
| 1034 | 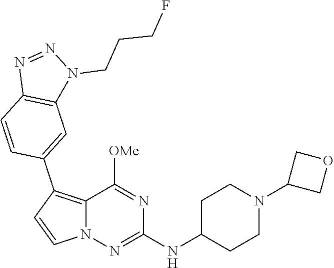 |
| 1035 | 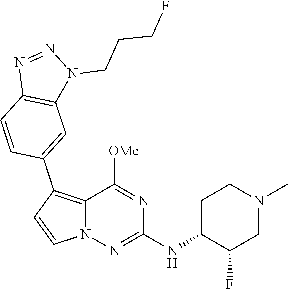 |
| 1036 | 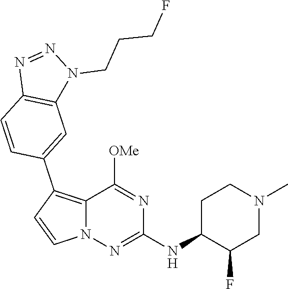 |
| 1037 | 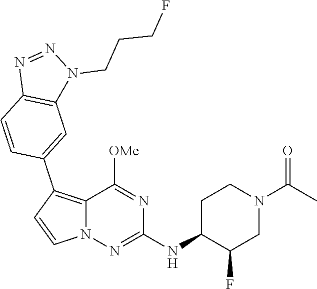 |
| 1038 | 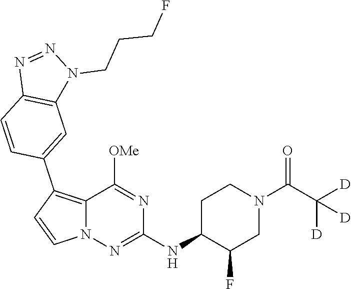 |
| 1039 | 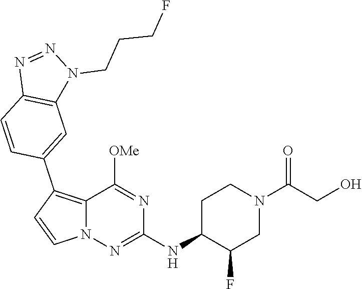 |
| 1040 | 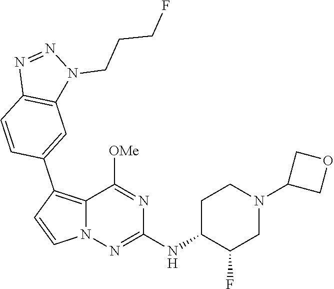 |
| 1041 | 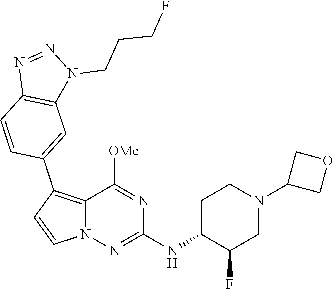 |
| 1042 | 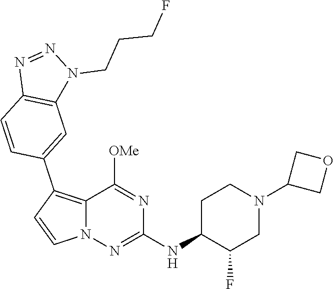 |
| 1043 | 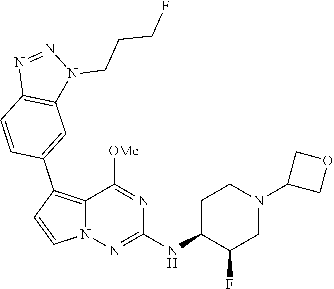 |
| 1044 | 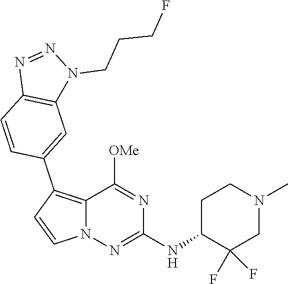 |
| 1045 | 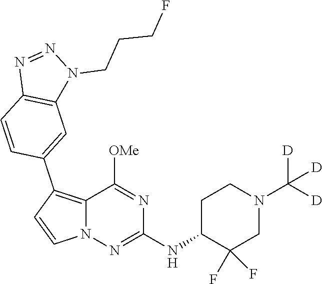 |
| 1046 | 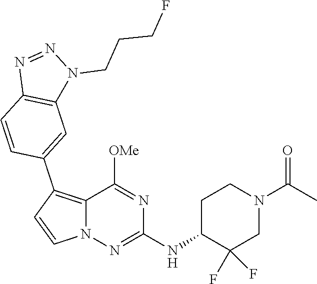 |
| 1047 | 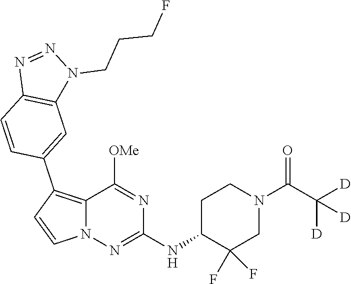 |
| 1048 | 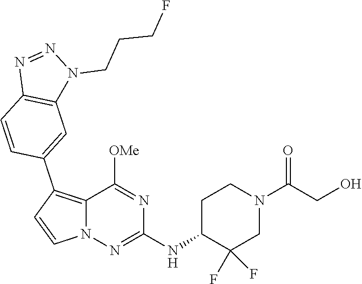 |
| 1049 | 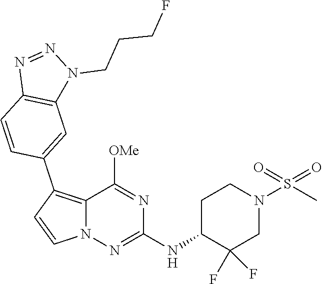 |
| 1050 | 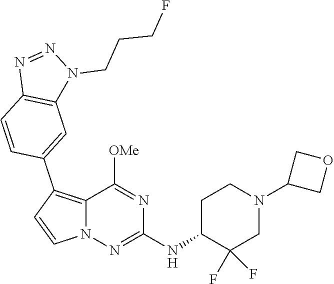 |
| 1051 | 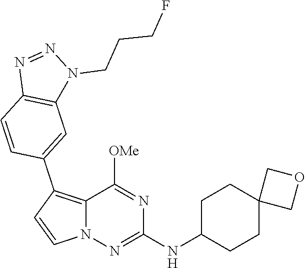 |
| 1052 | 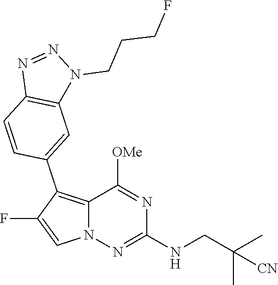 [SMILES: COc1nc(NCC(C)(C)C#N)nn2cc(F)c(-c3ccc4nnn(CCCF)c4c3)c12] |
| 1053 | 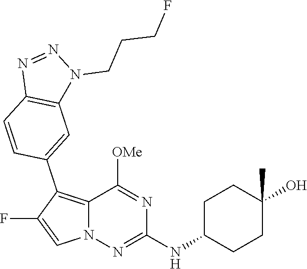 [SMILES: COc1nc(N[C@H]2CC[C@@](C)(O)CC2)nn2cc(F)c(-c3ccc4nnn(CCCF)c4c3)c12] |
| 1054 | 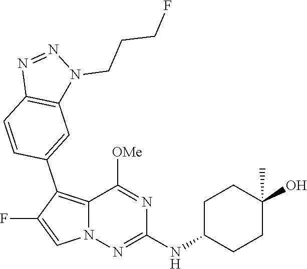 [SMILES: COc1nc(N[C@H]2CC[C@](C)(O)CC2)nn2cc(F)c(-c3ccc4nnn(CCCF)c4c3)c12] |
| 1055 | 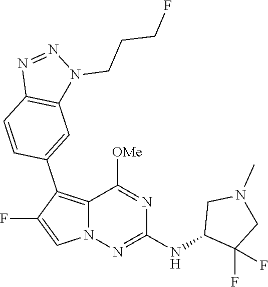 [SMILES: COc1nc(N[C@@H]2CN(C)CC2(F)F)nn2cc(F)c(-c3ccc4nnn(CCCF)c4c3)c12] |
| 1056 | 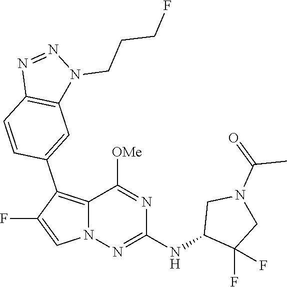 [SMILES: COc1nc(N[C@@H]2CN(C(C)=O)CC2(F)F)nn2cc(F)c(-c3ccc4nnn(CCCF)c4c3)c12] |
| 1057 | 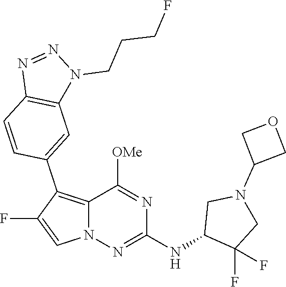 [SMILES: COc1nc(N[C@@H]2CN(C3COC3)CC2(F)F)nn2cc(F)c(-c3ccc4nnn(CCCF)c4c3)c12] |
| 1058 | 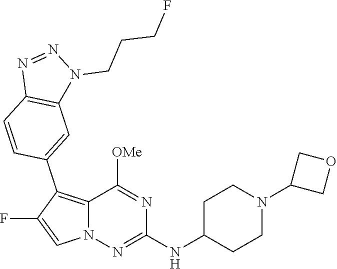 [SMILES: COc1nc(NC2CCN(C3COC3)CC2)nn2cc(F)c(-c3ccc4nnn(CCCF)c4c3)c12] |
| 1059 | 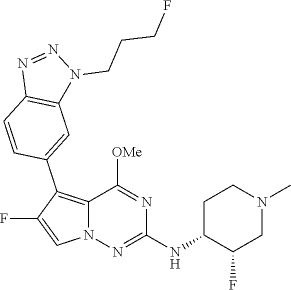 [SMILES: COc1nc(N[C@@H]2CCN(C)C[C@@H]2F)nn2cc(F)c(-c3ccc4nnn(CCCF)c4c3)c12] |
| 1060 | 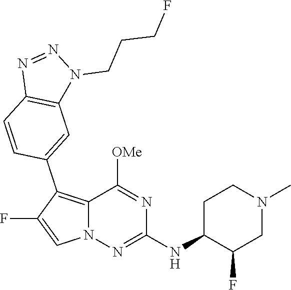 [SMILES: COc1nc(N[C@H]2CCN(C)C[C@H]2F)nn2cc(F)c(-c3ccc4nnn(CCCF)c4c3)c12] |
| 1061 | 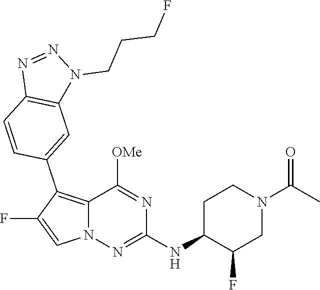 [SMILES: COc1nc(N[C@H]2CCN(C(C)=O)C[C@H]2F)nn2cc(F)c(-c3ccc4nnn(CCCF)c4c3)c12] |
| 1062 | 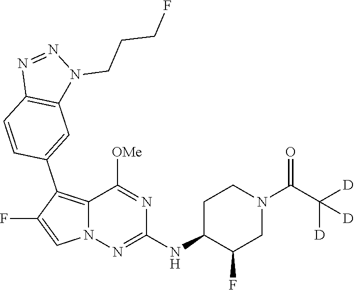 [SMILES: [2H]C([2H])([2H])C(=O)N1CC[C@H](Nc2nc(OC)c3c(-c4ccc5nnn(CCCF)c5c4)c(F)cn3n2)[C@H](F)C1] |
| 1063 | 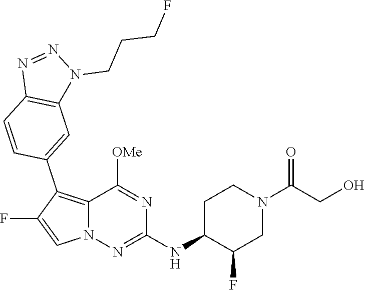 [SMILES: COc1nc(N[C@H]2CCN(C(=O)CO)C[C@H]2F)nn2cc(F)c(-c3ccc4nnn(CCCF)c4c3)c12] |
| 1064 | 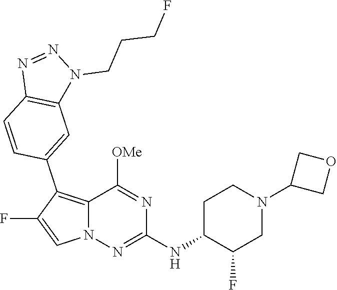 [SMILES: COc1nc(N[C@@H]2CCN(C3COC3)C[C@@H]2F)nn2cc(F)c(-c3ccc4nnn(CCCF)c4c3)c12] |
| 1065 | 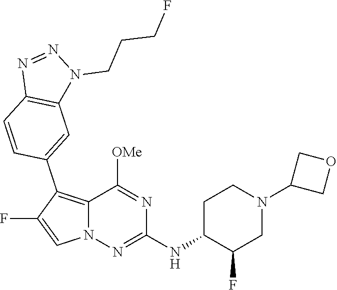 [SMILES: COc1nc(N[C@@H]2CCN(C3COC3)C[C@H]2F)nn2cc(F)c(-c3ccc4nnn(CCCF)c4c3)c12] |
| 1066 | 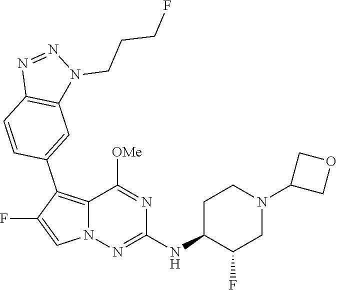 [SMILES: COc1nc(N[C@H]2CCN(C3COC3)C[C@@H]2F)nn2cc(F)c(-c3ccc4nnn(CCCF)c4c3)c12] |
| 1067 | 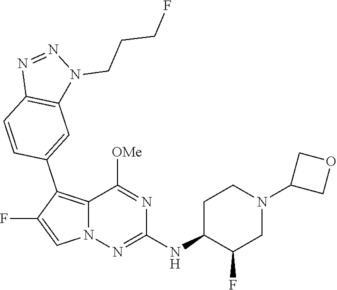 [SMILES: COc1nc(N[C@H]2CCN(C3COC3)C[C@H]2F)nn2cc(F)c(-c3ccc4nnn(CCCF)c4c3)c12] |
| 1068 | 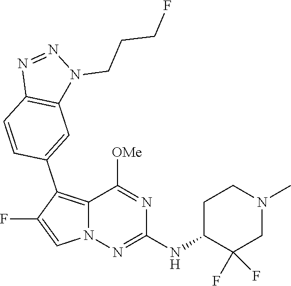 [SMILES: COc1nc(N[C@@H]2CCN(C)CC2(F)F)nn2cc(F)c(-c3ccc4nnn(CCCF)c4c3)c12] |
| 1069 | 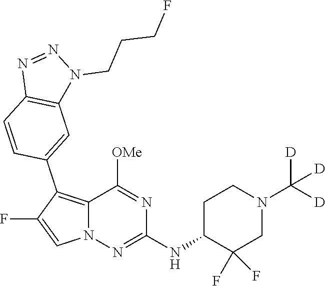 [SMILES: [2H]C([2H])([2H])N1CC[C@@H](Nc2nc(OC)c3c(-c4ccc5nnn(CCCF)c5c4)c(F)cn3n2)C(F)(F)C1] |
| 1070 | 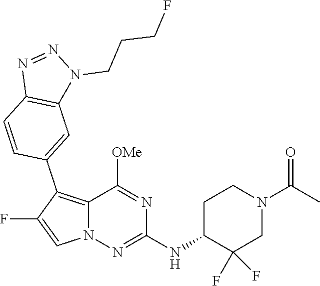 [SMILES: COc1nc(N[C@@H]2CCN(C(C)=O)CC2(F)F)nn2cc(F)c(-c3ccc4nnn(CCCF)c4c3)c12] |
| 1071 | 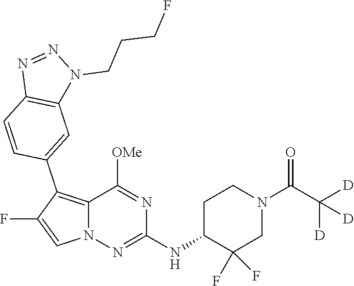 [SMILES: [2H]C([2H])([2H])C(=O)N1CC[C@@H](Nc2nc(OC)c3c(-c4ccc5nnn(CCCF)c5c4)c(F)cn3n2)C(F)(F)C1] |
| 1072 | 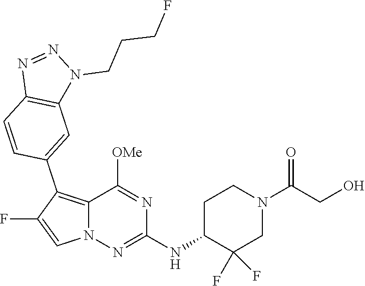 [SMILES: COc1nc(N[C@@H]2CCN(C(=O)CO)CC2(F)F)nn2cc(F)c(-c3ccc4nnn(CCCF)c4c3)c12] |
| 1073 | 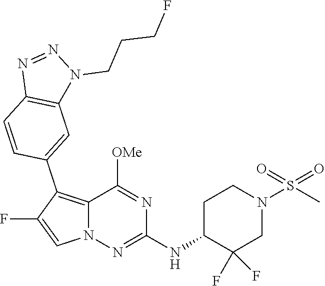 [SMILES: COc1nc(N[C@@H]2CCN(S(C)(=O)=O)CC2(F)F)nn2cc(F)c(-c3ccc4nnn(CCCF)c4c3)c12] |
| 1074 | 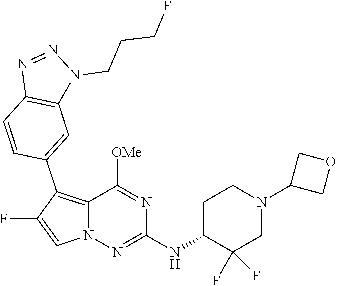 [SMILES: COc1nc(N[C@@H]2CCN(C3COC3)CC2(F)F)nn2cc(F)c(-c3ccc4nnn(CCCF)c4c3)c12] |
| 1075 | 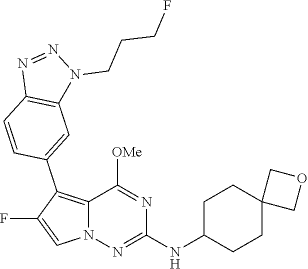 [SMILES: COc1nc(NC2CCC3(CC2)COC3)nn2cc(F)c(-c3ccc4nnn(CCCF)c4c3)c12] |
| 1076 | 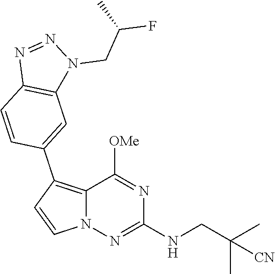 [SMILES: COc1nc(NCC(C)(C)C#N)nn2ccc(-c3ccc4nnn(C[C@H](C)F)c4c3)c12] |
| 1077 | 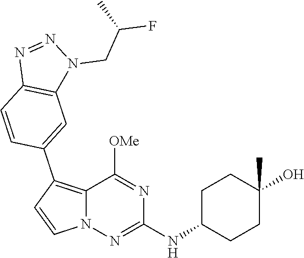 [SMILES: COc1nc(N[C@H]2CC[C@@](C)(O)CC2)nn2ccc(-c3ccc4nnn(C[C@H](C)F)c4c3)c12] |
| 1078 | 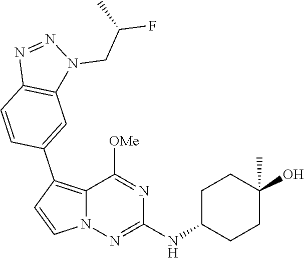 [SMILES: COc1nc(N[C@H]2CC[C@](C)(O)CC2)nn2ccc(-c3ccc4nnn(C[C@H](C)F)c4c3)c12] |
| 1079 | 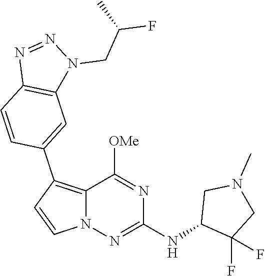 [SMILES: COc1nc(N[C@@H]2CN(C)CC2(F)F)nn2ccc(-c3ccc4nnn(C[C@H](C)F)c4c3)c12] |
| 1080 | 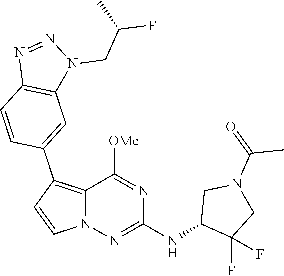 [SMILES: COc1nc(N[C@@H]2CN(C(C)=O)CC2(F)F)nn2ccc(-c3ccc4nnn(C[C@H](C)F)c4c3)c12] |
| 1081 | 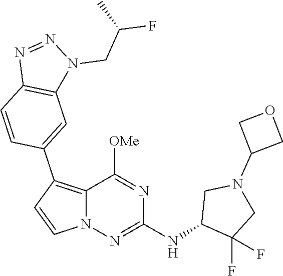 [SMILES: COc1nc(N[C@@H]2CN(C3COC3)CC2(F)F)nn2ccc(-c3ccc4nnn(C[C@H](C)F)c4c3)c12] |
| 1082 | 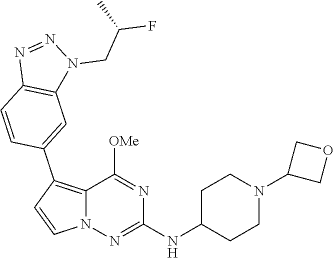 [SMILES: COc1nc(NC2CCN(C3COC3)CC2)nn2ccc(-c3ccc4nnn(C[C@H](C)F)c4c3)c12] |
| 1083 | 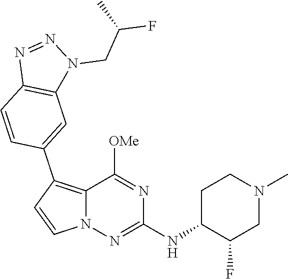 [SMILES: COc1nc(N[C@@H]2CCN(C)C[C@@H]2F)nn2ccc(-c3ccc4nnn(C[C@H](C)F)c4c3)c12] |
| 1084 | 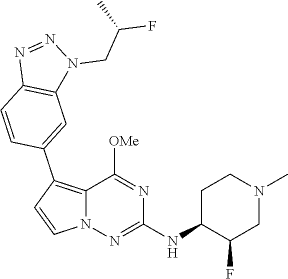 [SMILES: COc1nc(N[C@H]2CCN(C)C[C@H]2F)nn2ccc(-c3ccc4nnn(C[C@H](C)F)c4c3)c12] |
| 1085 | 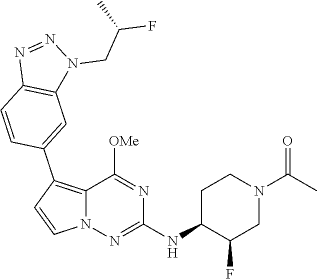 [SMILES: COc1nc(N[C@H]2CCN(C(C)=O)C[C@H]2F)nn2ccc(-c3ccc4nnn(C[C@H](C)F)c4c3)c12] |
| 1086 | 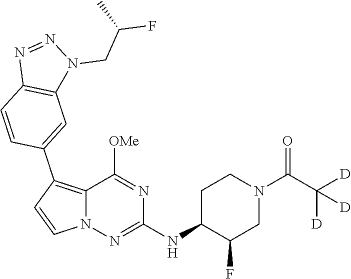 [SMILES: [2H]C([2H])([2H])C(=O)N1CC[C@H](Nc2nc(OC)c3c(-c4ccc5nnn(C[C@H](C)F)c5c4)ccn3n2)[C@H](F)C1] |
| 1087 | 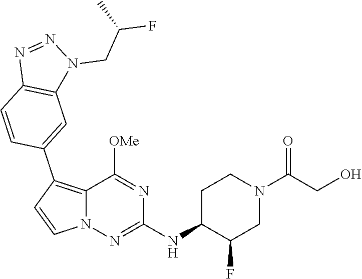 [SMILES: COc1nc(N[C@H]2CCN(C(=O)CO)C[C@H]2F)nn2ccc(-c3ccc4nnn(C[C@H](C)F)c4c3)c12] |
| 1088 | 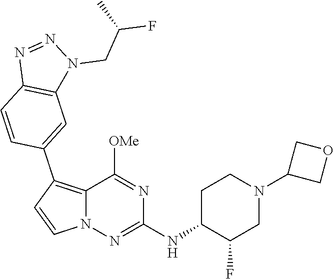 [SMILES: COc1nc(N[C@@H]2CCN(C3COC3)C[C@@H]2F)nn2ccc(-c3ccc4nnn(C[C@H](C)F)c4c3)c12] |
| 1089 | 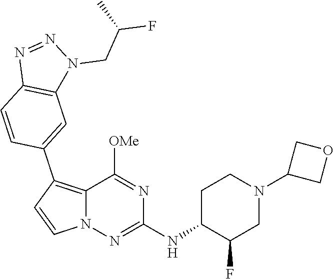 [SMILES: COc1nc(N[C@@H]2CCN(C3COC3)C[C@H]2F)nn2ccc(-c3ccc4nnn(C[C@H](C)F)c4c3)c12] |
| 1090 | 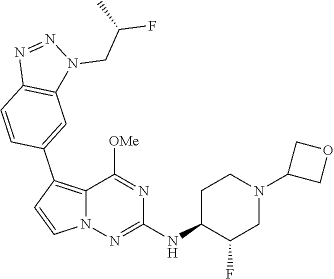 [SMILES: COc1nc(N[C@H]2CCN(C3COC3)C[C@@H]2F)nn2ccc(-c3ccc4nnn(C[C@H](C)F)c4c3)c12] |
| 1091 | 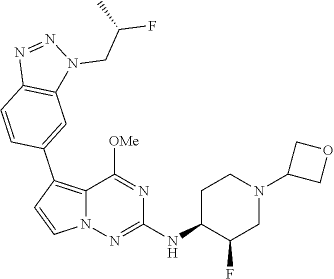 [SMILES: COc1nc(N[C@H]2CCN(C3COC3)C[C@H]2F)nn2ccc(-c3ccc4nnn(C[C@H](C)F)c4c3)c12] |
| 1092 | 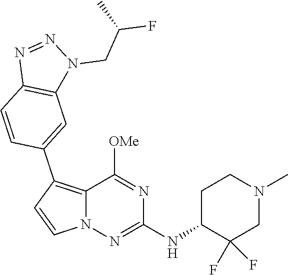 [SMILES: COc1nc(N[C@@H]2CCN(C)CC2(F)F)nn2ccc(-c3ccc4nnn(C[C@H](C)F)c4c3)c12] |
| 1093 | 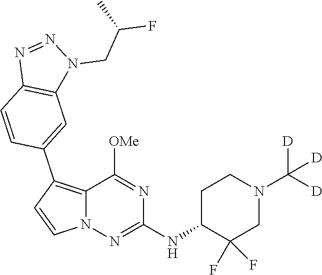 [SMILES: [2H]C([2H])([2H])N1CC[C@@H](Nc2nc(OC)c3c(-c4ccc5nnn(C[C@H](C)F)c5c4)ccn3n2)C(F)(F)C1] |
| 1094 | 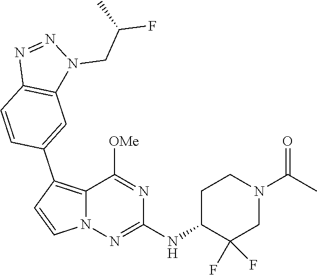 [SMILES: COc1nc(N[C@@H]2CCN(C(C)=O)CC2(F)F)nn2ccc(-c3ccc4nnn(C[C@H](C)F)c4c3)c12] |
| 1095 | 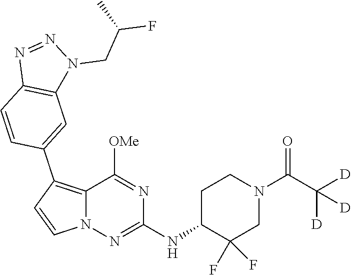 [SMILES: [2H]C([2H])([2H])C(=O)N1CC[C@@H](Nc2nc(OC)c3c(-c4ccc5nnn(C[C@H](C)F)c5c4)ccn3n2)C(F)(F)C1] |
| 1096 | 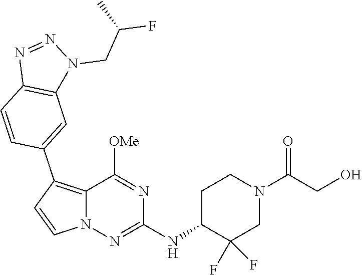 [SMILES: COc1nc(N[C@@H]2CCN(C(=O)CO)CC2(F)F)nn2ccc(-c3ccc4nnn(C[C@H](C)F)c4c3)c12] |
| 1097 | 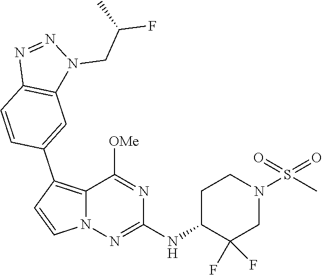 [SMILES: COc1nc(N[C@@H]2CCN(S(C)(=O)=O)CC2(F)F)nn2ccc(-c3ccc4nnn(C[C@H](C)F)c4c3)c12] |
| 1098 | 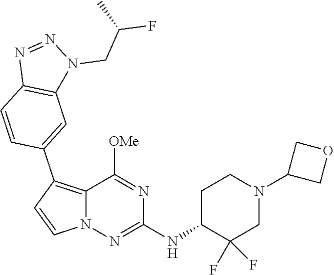 [SMILES: COc1nc(N[C@@H]2CCN(C3COC3)CC2(F)F)nn2ccc(-c3ccc4nnn(C[C@H](C)F)c4c3)c12] |
| 1099 | 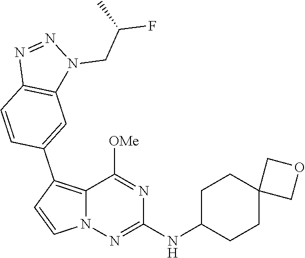 [SMILES: COc1nc(NC2CCC3(CC2)COC3)nn2ccc(-c3ccc4nnn(C[C@H](C)F)c4c3)c12] |
| 1100 | 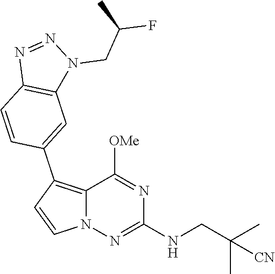 [SMILES: COc1nc(NCC(C)(C)C#N)nn2ccc(-c3ccc4nnn(C[C@@H](C)F)c4c3)c12] |
| 1101 | 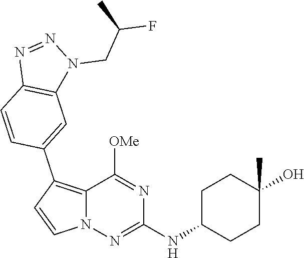 [SMILES: COc1nc(N[C@H]2CC[C@@](C)(O)CC2)nn2ccc(-c3ccc4nnn(C[C@@H](C)F)c4c3)c12] |
| 1102 | 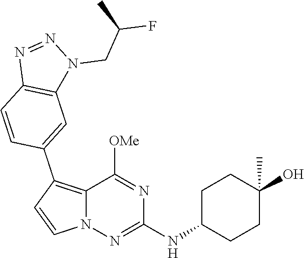 [SMILES: COc1nc(N[C@H]2CC[C@](C)(O)CC2)nn2ccc(-c3ccc4nnn(C[C@@H](C)F)c4c3)c12] |
| 1103 | 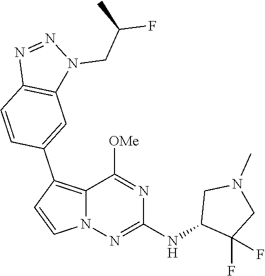 [SMILES: COc1nc(N[C@@H]2CN(C)CC2(F)F)nn2ccc(-c3ccc4nnn(C[C@@H](C)F)c4c3)c12] |
| 1104 | 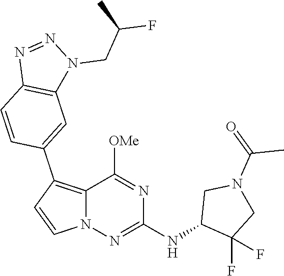 [SMILES: COc1nc(N[C@@H]2CN(C(C)=O)CC2(F)F)nn2ccc(-c3ccc4nnn(C[C@@H](C)F)c4c3)c12] |
| 1105 | 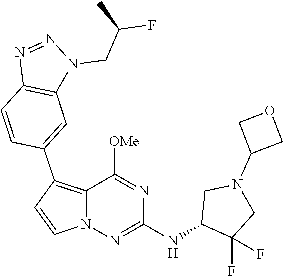 [SMILES: COc1nc(N[C@@H]2CN(C3COC3)CC2(F)F)nn2ccc(-c3ccc4nnn(C[C@@H](C)F)c4c3)c12] |
| 1106 | 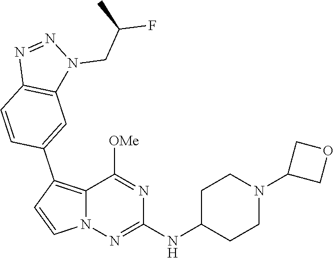 [SMILES: COc1nc(NC2CCN(C3COC3)CC2)nn2ccc(-c3ccc4nnn(C[C@@H](C)F)c4c3)c12] |
| 1107 | 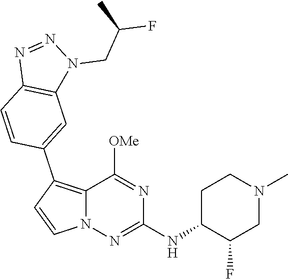 [SMILES: COc1nc(N[C@@H]2CCN(C)C[C@@H]2F)nn2ccc(-c3ccc4nnn(C[C@@H](C)F)c4c3)c12] |
| 1108 | 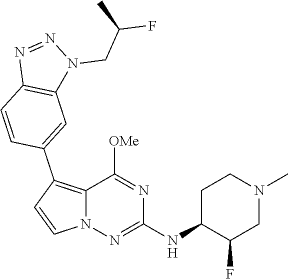 [SMILES: COc1nc(N[C@H]2CCN(C)C[C@H]2F)nn2ccc(-c3ccc4nnn(C[C@@H](C)F)c4c3)c12] |
| 1109 | 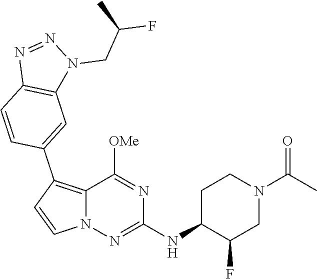 [SMILES: COc1nc(N[C@H]2CCN(C(C)=O)C[C@H]2F)nn2ccc(-c3ccc4nnn(C[C@@H](C)F)c4c3)c12] |
| 1110 | 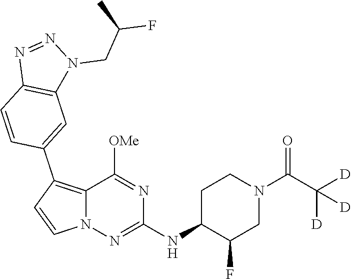 [SMILES: [2H]C([2H])([2H])C(=O)N1CC[C@H](Nc2nc(OC)c3c(-c4ccc5nnn(C[C@@H](C)F)c5c4)ccn3n2)[C@H](F)C1] |
| 1111 | 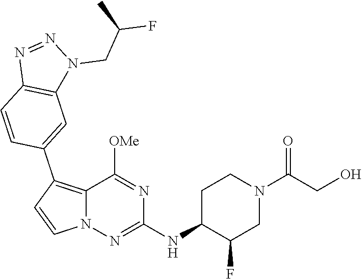 [SMILES: COc1nc(N[C@H]2CCN(C(=O)CO)C[C@H]2F)nn2ccc(-c3ccc4nnn(C[C@@H](C)F)c4c3)c12] |
| 1112 | 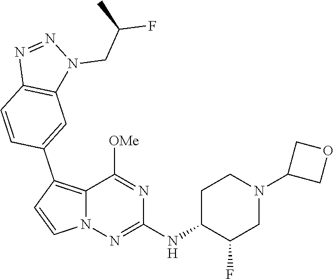 [SMILES: COc1nc(N[C@@H]2CCN(C3COC3)C[C@@H]2F)nn2ccc(-c3ccc4nnn(C[C@@H](C)F)c4c3)c12] |
| 1113 | 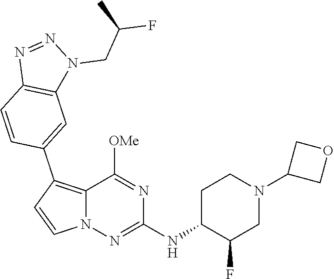 [SMILES: COc1nc(N[C@@H]2CCN(C3COC3)C[C@H]2F)nn2ccc(-c3ccc4nnn(C[C@@H](C)F)c4c3)c12] |
| 1114 | 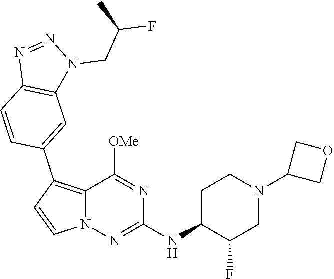 [SMILES: COc1nc(N[C@H]2CCN(C3COC3)C[C@@H]2F)nn2ccc(-c3ccc4nnn(C[C@@H](C)F)c4c3)c12] |
| 1115 | 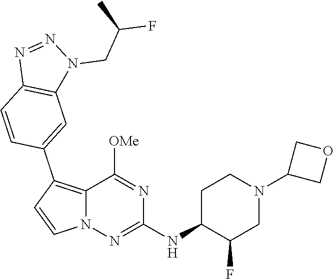 [SMILES: COc1nc(N[C@H]2CCN(C3COC3)C[C@H]2F)nn2ccc(-c3ccc4nnn(C[C@@H](C)F)c4c3)c12] |
| 1116 | 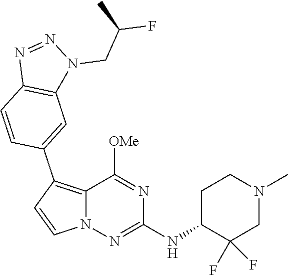 [SMILES: COc1nc(N[C@@H]2CCN(C)CC2(F)F)nn2ccc(-c3ccc4nnn(C[C@@H](C)F)c4c3)c12] |
| 1117 | 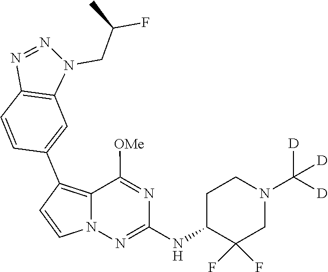 [SMILES: [2H]C([2H])([2H])N1CC[C@@H](Nc2nc(OC)c3c(-c4ccc5nnn(C[C@@H](C)F)c5c4)ccn3n2)C(F)(F)C1] |
| 1118 | 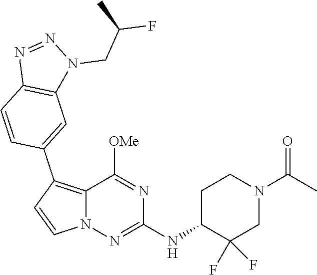 [SMILES: COc1nc(N[C@@H]2CCN(C(C)=O)CC2(F)F)nn2ccc(-c3ccc4nnn(C[C@@H](C)F)c4c3)c12] |
| 1119 | 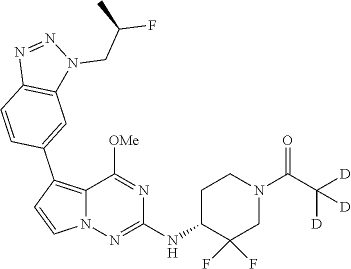 [SMILES: [2H]C([2H])([2H])C(=O)N1CC[C@@H](Nc2nc(OC)c3c(-c4ccc5nnn(C[C@@H](C)F)c5c4)ccn3n2)C(F)(F)C1] |
| 1120 | 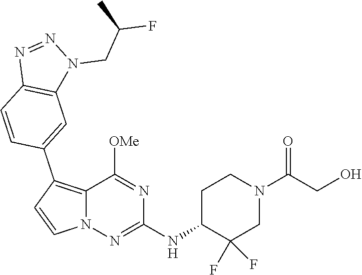 [SMILES: COc1nc(N[C@@H]2CCN(C(=O)CO)CC2(F)F)nn2ccc(-c3ccc4nnn(C[C@@H](C)F)c4c3)c12] |
| 1121 | 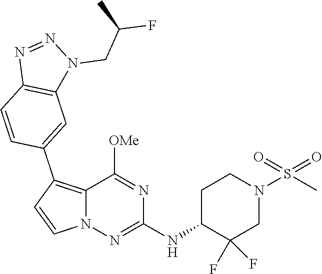 [SMILES: COc1nc(N[C@@H]2CCN(S(C)(=O)=O)CC2(F)F)nn2ccc(-c3ccc4nnn(C[C@@H](C)F)c4c3)c12] |
| 1122 | 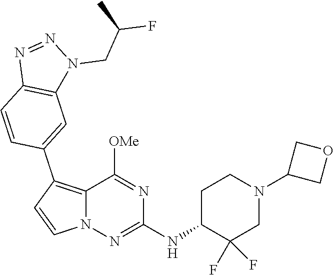 [SMILES: COc1nc(N[C@@H]2CCN(C3COC3)CC2(F)F)nn2ccc(-c3ccc4nnn(C[C@@H](C)F)c4c3)c12] |
| 1123 | 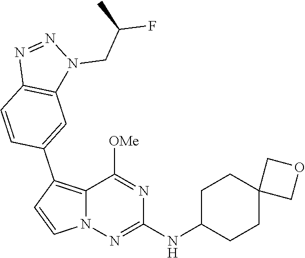 [SMILES: COc1nc(NC2CCC3(CC2)COC3)nn2ccc(-c3ccc4nnn(C[C@@H](C)F)c4c3)c12] |
| 1124 | 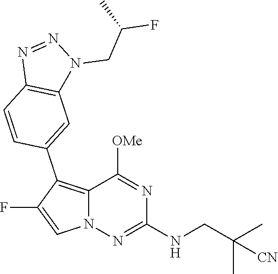 [SMILES: COc1nc(NCC(C)(C)C#N)nn2cc(F)c(-c3ccc4nnn(C[C@H](C)F)c4c3)c12] |
| 1125 | 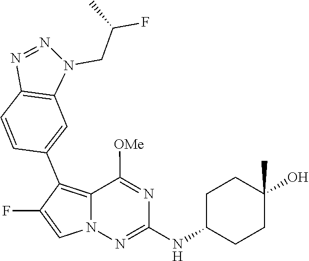 [SMILES: COc1nc(N[C@H]2CC[C@@](C)(O)CC2)nn2cc(F)c(-c3ccc4nnn(C[C@H](C)F)c4c3)c12] |
| 1126 | 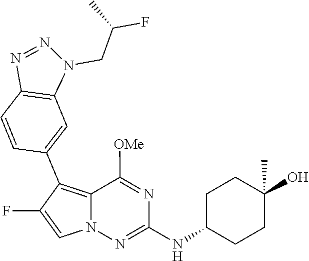 [SMILES: COc1nc(N[C@H]2CC[C@](C)(O)CC2)nn2cc(F)c(-c3ccc4nnn(C[C@H](C)F)c4c3)c12] |
| 1127 | 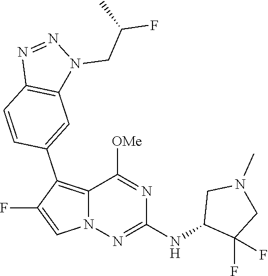 [SMILES: COc1nc(N[C@@H]2CN(C)CC2(F)F)nn2cc(F)c(-c3ccc4nnn(C[C@H](C)F)c4c3)c12] |
| 1128 | 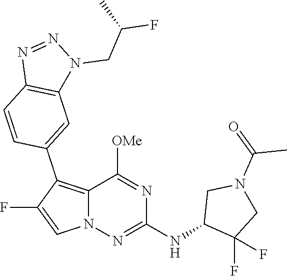 [SMILES: COc1nc(N[C@@H]2CN(C(C)=O)CC2(F)F)nn2cc(F)c(-c3ccc4nnn(C[C@H](C)F)c4c3)c12] |
| 1129 | 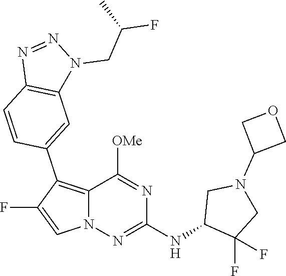 [SMILES: COc1nc(N[C@@H]2CN(C3COC3)CC2(F)F)nn2cc(F)c(-c3ccc4nnn(C[C@H](C)F)c4c3)c12] |
| 1130 | 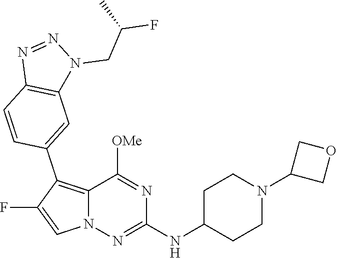 [SMILES: COc1nc(NC2CCN(C3COC3)CC2)nn2cc(F)c(-c3ccc4nnn(C[C@H](C)F)c4c3)c12] |
| 1131 | 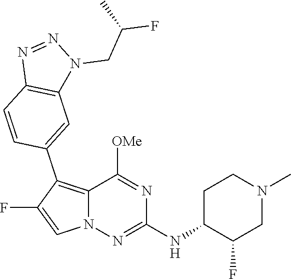 [SMILES: COc1nc(N[C@@H]2CCN(C)C[C@@H]2F)nn2cc(F)c(-c3ccc4nnn(C[C@H](C)F)c4c3)c12] |
| 1132 | 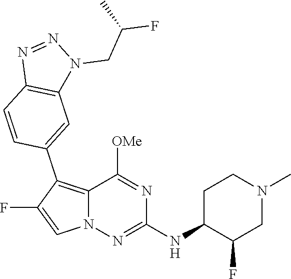 [SMILES: COc1nc(N[C@H]2CCN(C)C[C@H]2F)nn2cc(F)c(-c3ccc4nnn(C[C@H](C)F)c4c3)c12] |
| 1133 | 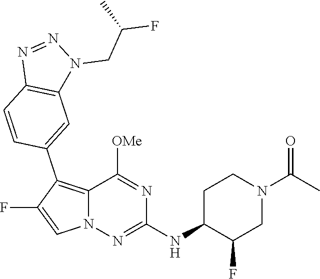 [SMILES: COc1nc(N[C@H]2CCN(C(C)=O)C[C@H]2F)nn2cc(F)c(-c3ccc4nnn(C[C@H](C)F)c4c3)c12] |
| 1134 | 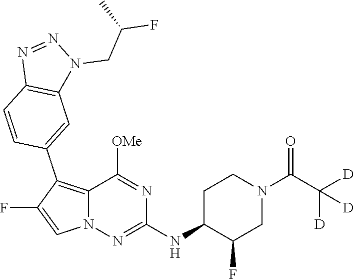 [SMILES: [2H]C([2H])([2H])C(=O)N1CC[C@H](Nc2nc(OC)c3c(-c4ccc5nnn(C[C@H](C)F)c5c4)c(F)cn3n2)[C@H](F)C1] |
| 1135 | 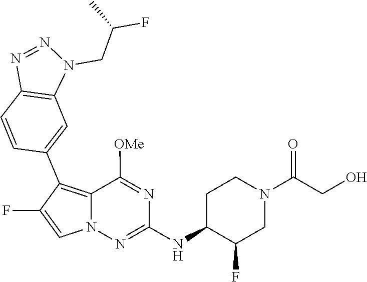 [SMILES: COc1nc(N[C@H]2CCN(C(=O)CO)C[C@H]2F)nn2cc(F)c(-c3ccc4nnn(C[C@H](C)F)c4c3)c12] |
| 1136 | 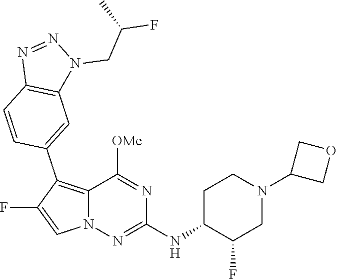 [SMILES: COc1nc(N[C@@H]2CCN(C3COC3)C[C@@H]2F)nn2cc(F)c(-c3ccc4nnn(C[C@H](C)F)c4c3)c12] |
| 1137 | 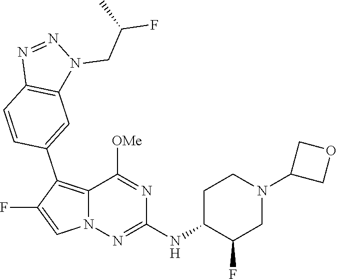 [SMILES: COc1nc(N[C@@H]2CCN(C3COC3)C[C@H]2F)nn2cc(F)c(-c3ccc4nnn(C[C@H](C)F)c4c3)c12] |
| 1138 | 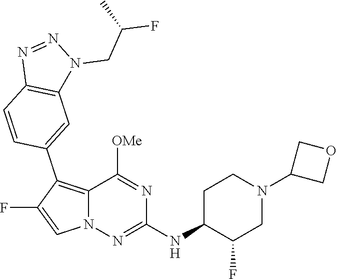 [SMILES: COc1nc(N[C@H]2CCN(C3COC3)C[C@@H]2F)nn2cc(F)c(-c3ccc4nnn(C[C@H](C)F)c4c3)c12] |
| 1139 | 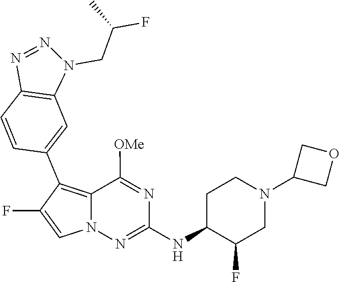 [SMILES: COc1nc(N[C@H]2CCN(C3COC3)C[C@H]2F)nn2cc(F)c(-c3ccc4nnn(C[C@H](C)F)c4c3)c12] |
| 1140 | 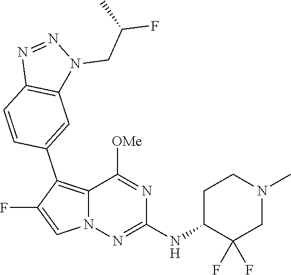 [SMILES: COc1nc(N[C@@H]2CCN(C)CC2(F)F)nn2cc(F)c(-c3ccc4nnn(C[C@H](C)F)c4c3)c12] |
| 1141 | 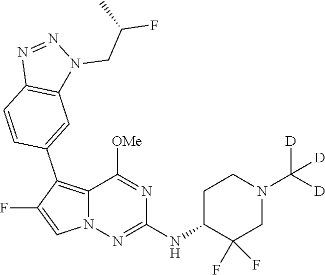 [SMILES: [2H]C([2H])([2H])N1CC[C@@H](Nc2nc(OC)c3c(-c4ccc5nnn(C[C@H](C)F)c5c4)c(F)cn3n2)C(F)(F)C1] |
| 1142 | 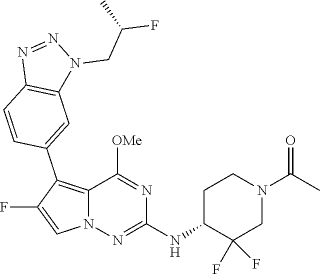 [SMILES: COc1nc(N[C@@H]2CCN(C(C)=O)CC2(F)F)nn2cc(F)c(-c3ccc4nnn(C[C@H](C)F)c4c3)c12] |
| 1143 | 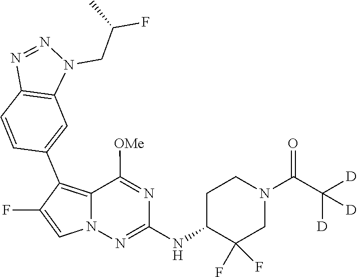 [SMILES: [2H]C([2H])([2H])C(=O)N1CC[C@@H](Nc2nc(OC)c3c(-c4ccc5nnn(C[C@H](C)F)c5c4)c(F)cn3n2)C(F)(F)C1] |
| 1144 | 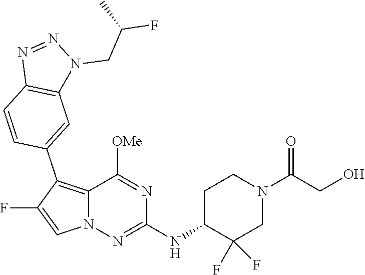 [SMILES: COc1nc(N[C@@H]2CCN(C(=O)CO)CC2(F)F)nn2cc(F)c(-c3ccc4nnn(C[C@H](C)F)c4c3)c12] |
| 1145 | 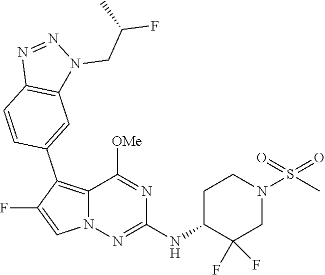 [SMILES: COc1nc(N[C@@H]2CCN(S(C)(=O)=O)CC2(F)F)nn2cc(F)c(-c3ccc4nnn(C[C@H](C)F)c4c3)c12] |
| 1146 | 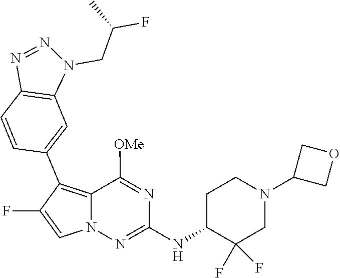 [SMILES: COc1nc(N[C@@H]2CCN(C3COC3)CC2(F)F)nn2cc(F)c(-c3ccc4nnn(C[C@H](C)F)c4c3)c12] |
| 1147 | 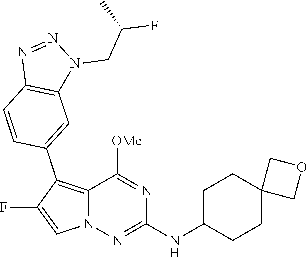 [SMILES: COc1nc(NC2CCC3(CC2)COC3)nn2cc(F)c(-c3ccc4nnn(C[C@H](C)F)c4c3)c12] |
| 1148 | 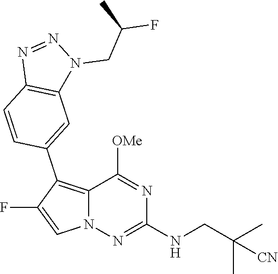 [SMILES: COc1nc(NCC(C)(C)C#N)nn2cc(F)c(-c3ccc4nnn(C[C@@H](C)F)c4c3)c12] |
| 1149 | 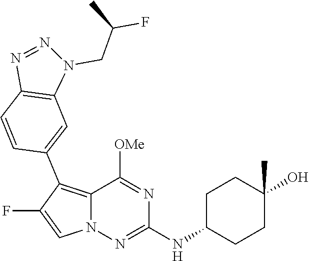 [SMILES: COc1nc(N[C@H]2CC[C@@](C)(O)CC2)nn2cc(F)c(-c3ccc4nnn(C[C@@H](C)F)c4c3)c12] |
| 1150 | 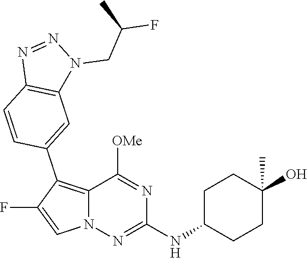 [SMILES: COc1nc(N[C@H]2CC[C@](C)(O)CC2)nn2cc(F)c(-c3ccc4nnn(C[C@@H](C)F)c4c3)c12] |
| 1151 | 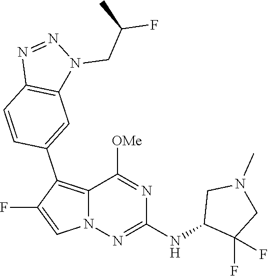 [SMILES: COc1nc(N[C@@H]2CN(C)CC2(F)F)nn2cc(F)c(-c3ccc4nnn(C[C@@H](C)F)c4c3)c12] |
| 1152 | 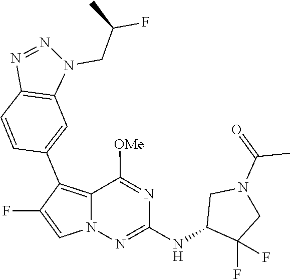 [SMILES: COc1nc(N[C@@H]2CN(C(C)=O)CC2(F)F)nn2cc(F)c(-c3ccc4nnn(C[C@@H](C)F)c4c3)c12] |
| 1153 | 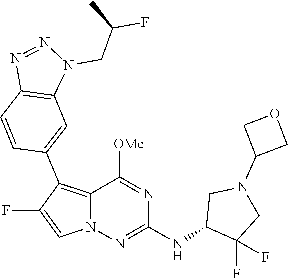 [SMILES: COc1nc(N[C@@H]2CN(C3COC3)CC2(F)F)nn2cc(F)c(-c3ccc4nnn(C[C@@H](C)F)c4c3)c12] |
| 1154 | 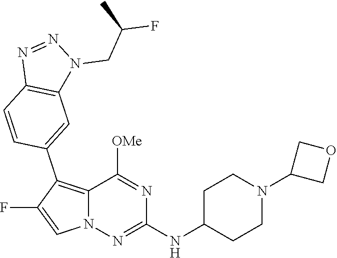 [SMILES: COc1nc(NC2CCN(C3COC3)CC2)nn2cc(F)c(-c3ccc4nnn(C[C@@H](C)F)c4c3)c12] |
| 1155 | 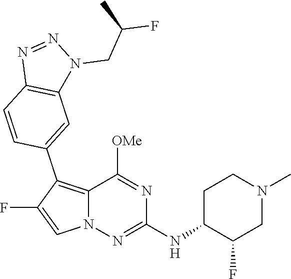 [SMILES: COc1nc(N[C@@H]2CCN(C)C[C@@H]2F)nn2cc(F)c(-c3ccc4nnn(C[C@@H](C)F)c4c3)c12] |
| 1156 | 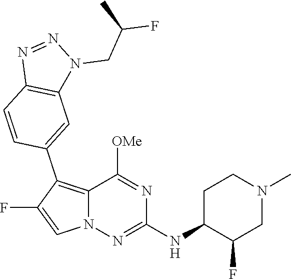 [SMILES: COc1nc(N[C@H]2CCN(C)C[C@H]2F)nn2cc(F)c(-c3ccc4nnn(C[C@@H](C)F)c4c3)c12] |
| 1157 | 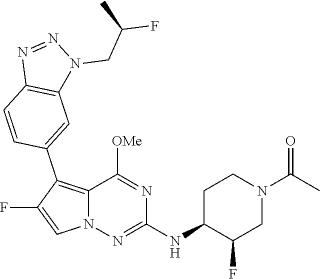 [SMILES: COc1nc(N[C@H]2CCN(C(C)=O)C[C@H]2F)nn2cc(F)c(-c3ccc4nnn(C[C@@H](C)F)c4c3)c12] |
| 1158 | 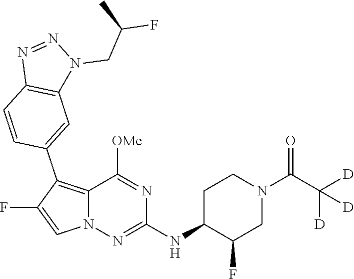 [SMILES: [2H]C([2H])([2H])C(=O)N1CC[C@H](Nc2nc(OC)c3c(-c4ccc5nnn(C[C@@H](C)F)c5c4)c(F)cn3n2)[C@H](F)C1] |
| 1159 | 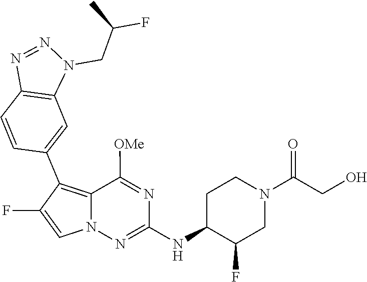 [SMILES: COc1nc(N[C@H]2CCN(C(=O)CO)C[C@H]2F)nn2cc(F)c(-c3ccc4nnn(C[C@@H](C)F)c4c3)c12] |
| 1160 | 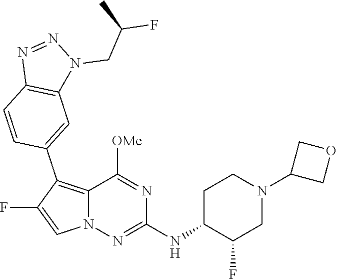 [SMILES: COc1nc(N[C@@H]2CCN(C3COC3)C[C@@H]2F)nn2cc(F)c(-c3ccc4nnn(C[C@@H](C)F)c4c3)c12] |
| 1161 | 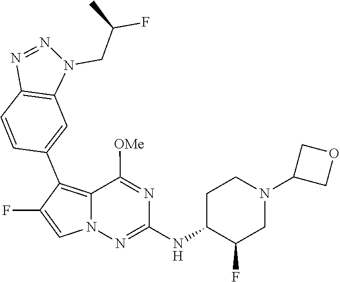 [SMILES: COc1nc(N[C@@H]2CCN(C3COC3)C[C@H]2F)nn2cc(F)c(-c3ccc4nnn(C[C@@H](C)F)c4c3)c12] |
| 1162 | 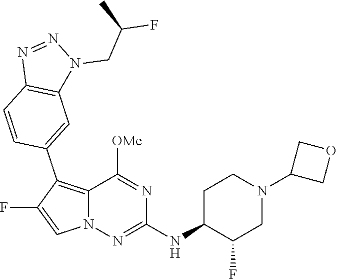 [SMILES: COc1nc(N[C@H]2CCN(C3COC3)C[C@@H]2F)nn2cc(F)c(-c3ccc4nnn(C[C@@H](C)F)c4c3)c12] |
| 1163 | 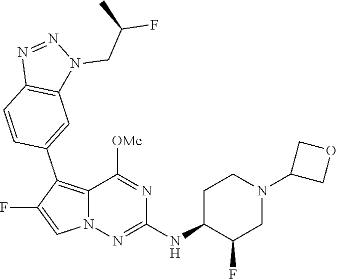 [SMILES: COc1nc(N[C@H]2CCN(C3COC3)C[C@H]2F)nn2cc(F)c(-c3ccc4nnn(C[C@@H](C)F)c4c3)c12] |
| 1164 | 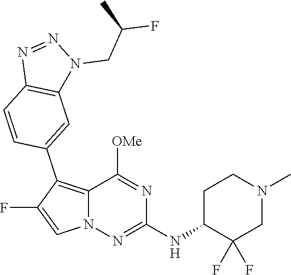 [SMILES: COc1nc(N[C@@H]2CCN(C)CC2(F)F)nn2cc(F)c(-c3ccc4nnn(C[C@@H](C)F)c4c3)c12] |
| 1165 | 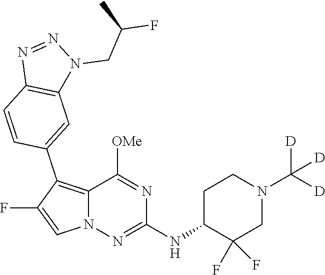 [SMILES: [2H]C([2H])([2H])N1CC[C@@H](Nc2nc(OC)c3c(-c4ccc5nnn(C[C@@H](C)F)c5c4)c(F)cn3n2)C(F)(F)C1] |
| 1166 | 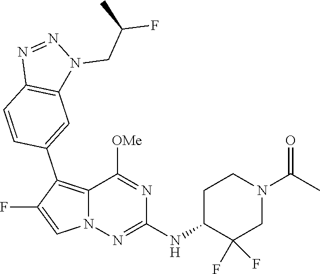 [SMILES: COc1nc(N[C@@H]2CCN(C(C)=O)CC2(F)F)nn2cc(F)c(-c3ccc4nnn(C[C@@H](C)F)c4c3)c12] |
| 1167 | 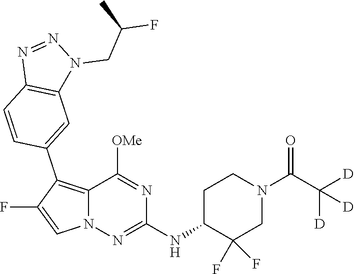 [SMILES: [2H]C([2H])([2H])C(=O)N1CC[C@@H](Nc2nc(OC)c3c(-c4ccc5nnn(C[C@@H](C)F)c5c4)c(F)cn3n2)C(F)(F)C1] |
| 1168 | 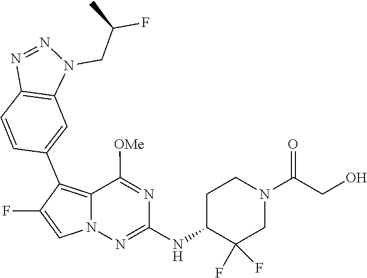 [SMILES: COc1nc(N[C@@H]2CCN(C(=O)CO)CC2(F)F)nn2cc(F)c(-c3ccc4nnn(C[C@@H](C)F)c4c3)c12] |
| 1169 | 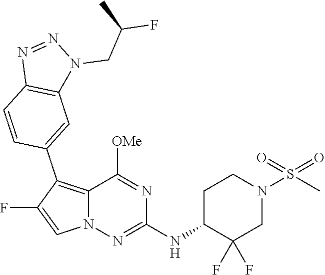 [SMILES: COc1nc(N[C@@H]2CCN(S(C)(=O)=O)CC2(F)F)nn2cc(F)c(-c3ccc4nnn(C[C@@H](C)F)c4c3)c12] |
| 1170 | 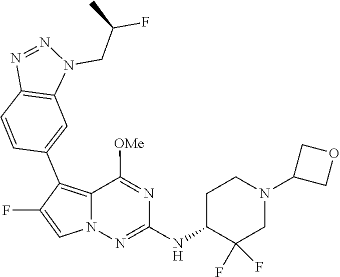 [SMILES: COc1nc(N[C@@H]2CCN(C3COC3)CC2(F)F)nn2cc(F)c(-c3ccc4nnn(C[C@@H](C)F)c4c3)c12] |
| 1171 | 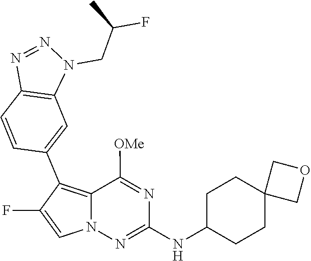 [SMILES: COc1nc(NC2CCC3(CC2)COC3)nn2cc(F)c(-c3ccc4nnn(C[C@@H](C)F)c4c3)c12] |
| 1172 | 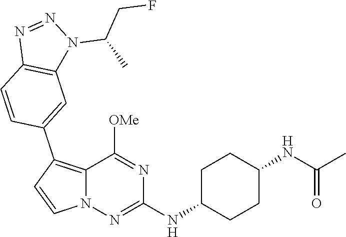 [SMILES: COc1nc(N[C@H]2CC[C@@H](NC(C)=O)CC2)nn2ccc(-c3ccc4nnn([C@@H](C)CF)c4c3)c12] |
| 1173 | 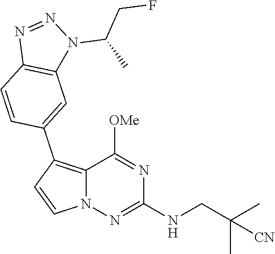 [SMILES: COc1nc(NCC(C)(C)C#N)nn2ccc(-c3ccc4nnn([C@@H](C)CF)c4c3)c12] |
| 1174 | 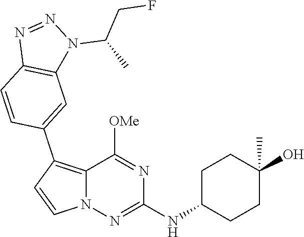 [SMILES: COc1nc(N[C@H]2CC[C@](C)(O)CC2)nn2ccc(-c3ccc4nnn([C@@H](C)CF)c4c3)c12] |
| 1175 | 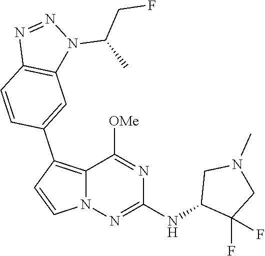 [SMILES: COc1nc(N[C@@H]2CN(C)CC2(F)F)nn2ccc(-c3ccc4nnn([C@@H](C)CF)c4c3)c12] |
| 1176 | 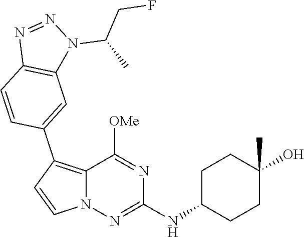 [SMILES: COc1nc(N[C@H]2CC[C@@](C)(O)CC2)nn2ccc(-c3ccc4nnn([C@@H](C)CF)c4c3)c12] |
| 1177 | 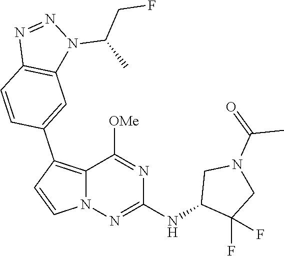 [SMILES: COc1nc(N[C@@H]2CN(C(C)=O)CC2(F)F)nn2ccc(-c3ccc4nnn([C@@H](C)CF)c4c3)c12] |
| 1178 | 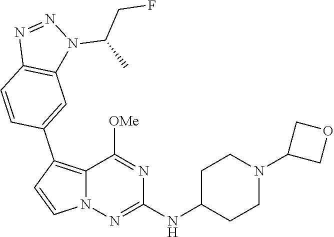 [SMILES: COc1nc(NC2CCN(C3COC3)CC2)nn2ccc(-c3ccc4nnn([C@@H](C)CF)c4c3)c12] |
| 1179 | 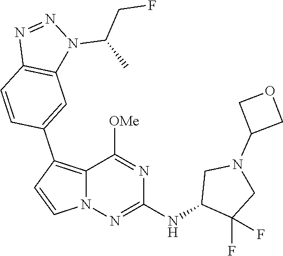 [SMILES: COc1nc(N[C@@H]2CN(C3COC3)CC2(F)F)nn2ccc(-c3ccc4nnn([C@@H](C)CF)c4c3)c12] |
| 1180 | 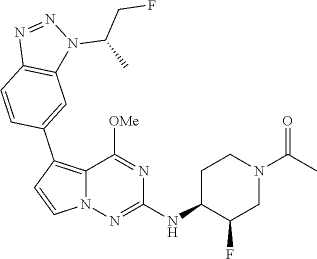 [SMILES: COc1nc(N[C@H]2CCN(C(C)=O)C[C@H]2F)nn2ccc(-c3ccc4nnn([C@@H](C)CF)c4c3)c12] |
| 1181 | 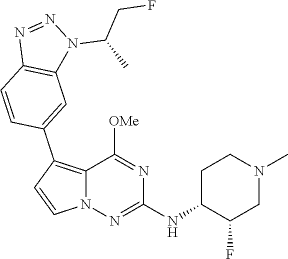 [SMILES: COc1nc(N[C@@H]2CCN(C)C[C@@H]2F)nn2ccc(-c3ccc4nnn([C@@H](C)CF)c4c3)c12] |
| 1182 | 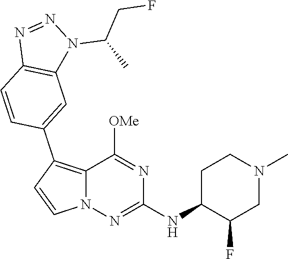 [SMILES: COc1nc(N[C@H]2CCN(C)C[C@H]2F)nn2ccc(-c3ccc4nnn([C@@H](C)CF)c4c3)c12] |
| 1183 | 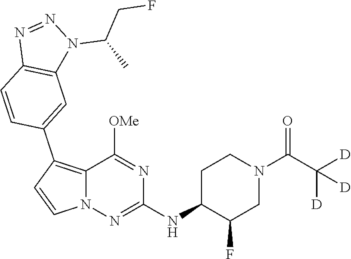 [SMILES: [2H]C([2H])([2H])C(=O)N1CC[C@H](Nc2nc(OC)c3c(-c4ccc5nnn([C@@H](C)CF)c5c4)ccn3n2)[C@H](F)C1] |
| 1184 | 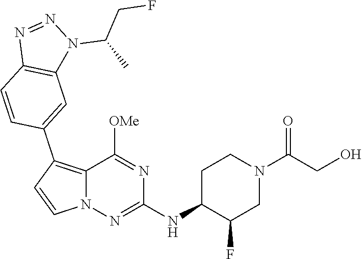 [SMILES: COc1nc(N[C@H]2CCN(C(=O)CO)C[C@H]2F)nn2ccc(-c3ccc4nnn([C@@H](C)CF)c4c3)c12] |
| 1185 | 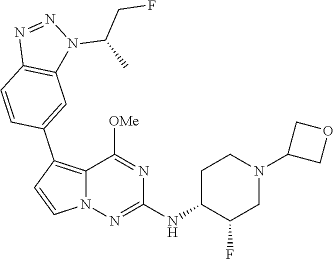 [SMILES: COc1nc(N[C@@H]2CCN(C3COC3)C[C@@H]2F)nn2ccc(-c3ccc4nnn([C@@H](C)CF)c4c3)c12] |
| 1186 | 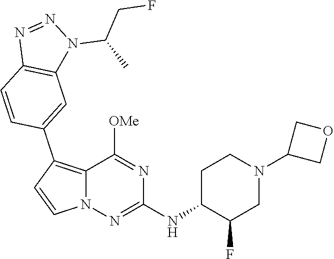 [SMILES: COc1nc(N[C@@H]2CCN(C3COC3)C[C@H]2F)nn2ccc(-c3ccc4nnn([C@@H](C)CF)c4c3)c12] |
| 1187 | 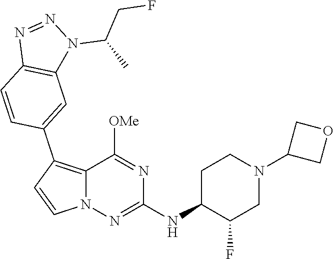 [SMILES: COc1nc(N[C@H]2CCN(C3COC3)C[C@@H]2F)nn2ccc(-c3ccc4nnn([C@@H](C)CF)c4c3)c12] |
| 1188 | 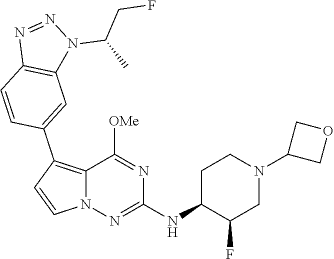 [SMILES: COc1nc(N[C@H]2CCN(C3COC3)C[C@H]2F)nn2ccc(-c3ccc4nnn([C@@H](C)CF)c4c3)c12] |
| 1189 | 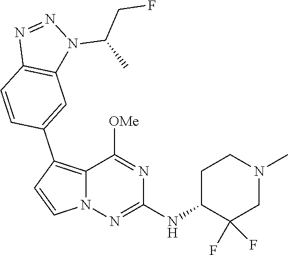 [SMILES: COc1nc(N[C@@H]2CCN(C)CC2(F)F)nn2ccc(-c3ccc4nnn([C@@H](C)CF)c4c3)c12] |
| 1190 | 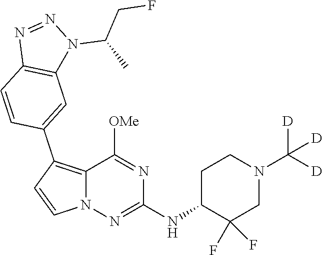 [SMILES: [2H]C([2H])([2H])N1CC[C@@H](Nc2nc(OC)c3c(-c4ccc5nnn([C@@H](C)CF)c5c4)ccn3n2)C(F)(F)C1] |
| 1191 | 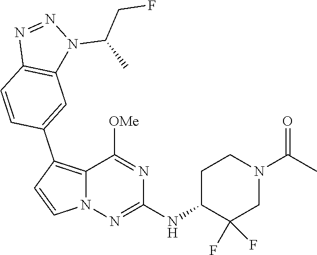 [SMILES: COc1nc(N[C@@H]2CCN(C(C)=O)CC2(F)F)nn2ccc(-c3ccc4nnn([C@@H](C)CF)c4c3)c12] |
| 1192 | 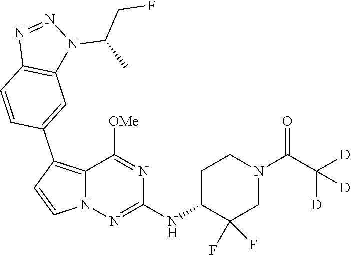 [SMILES: [2H]C([2H])([2H])C(=O)N1CC[C@@H](Nc2nc(OC)c3c(-c4ccc5nnn([C@@H](C)CF)c5c4)ccn3n2)C(F)(F)C1] |
| 1193 | 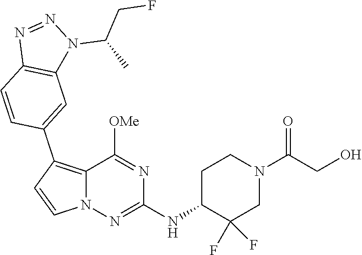 [SMILES: COc1nc(N[C@@H]2CCN(C(=O)CO)CC2(F)F)nn2ccc(-c3ccc4nnn([C@@H](C)CF)c4c3)c12] |
| 1194 | 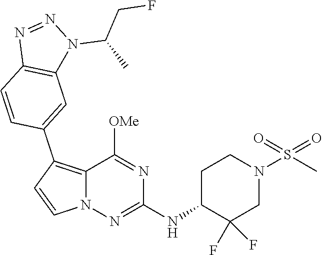 [SMILES: COc1nc(N[C@@H]2CCN(S(C)(=O)=O)CC2(F)F)nn2ccc(-c3ccc4nnn([C@@H](C)CF)c4c3)c12] |
| 1195 | 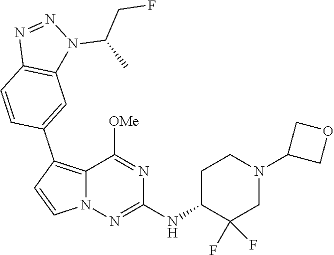 [SMILES: COc1nc(N[C@@H]2CCN(C3COC3)CC2(F)F)nn2ccc(-c3ccc4nnn([C@@H](C)CF)c4c3)c12] |
| 1196 | 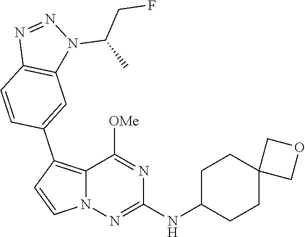 [SMILES: COc1nc(NC2CCC3(CC2)COC3)nn2ccc(-c3ccc4nnn([C@@H](C)CF)c4c3)c12] |
| 1197 | 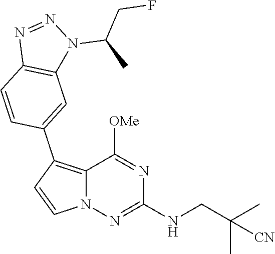 [SMILES: COc1nc(NCC(C)(C)C#N)nn2ccc(-c3ccc4nnn([C@H](C)CF)c4c3)c12] |
| 1198 | 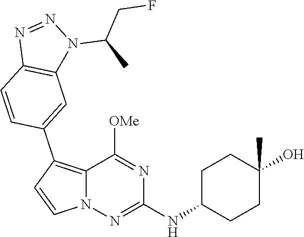 [SMILES: COc1nc(N[C@H]2CC[C@@](C)(O)CC2)nn2ccc(-c3ccc4nnn([C@H](C)CF)c4c3)c12] |
| 1199 | 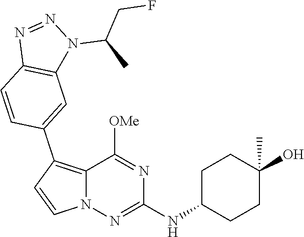 [SMILES: COc1nc(N[C@H]2CC[C@](C)(O)CC2)nn2ccc(-c3ccc4nnn([C@H](C)CF)c4c3)c12] |
| 1200 | 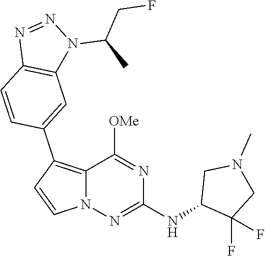 [SMILES: COc1nc(N[C@@H]2CN(C)CC2(F)F)nn2ccc(-c3ccc4nnn([C@H](C)CF)c4c3)c12] |
| 1201 | 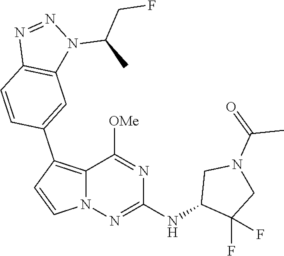 [SMILES: COc1nc(N[C@@H]2CN(C(C)=O)CC2(F)F)nn2ccc(-c3ccc4nnn([C@H](C)CF)c4c3)c12] |
| 1202 | 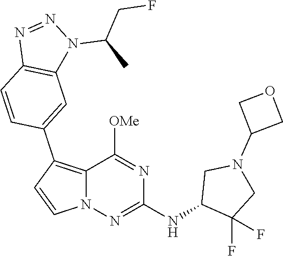 [SMILES: COc1nc(N[C@@H]2CN(C3COC3)CC2(F)F)nn2ccc(-c3ccc4nnn([C@H](C)CF)c4c3)c12] |
| 1203 | 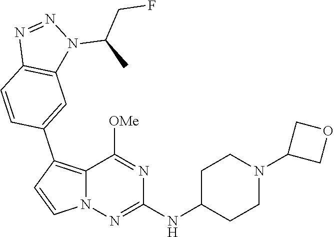 [SMILES: COc1nc(NC2CCN(C3COC3)CC2)nn2ccc(-c3ccc4nnn([C@H](C)CF)c4c3)c12] |
| 1204 | 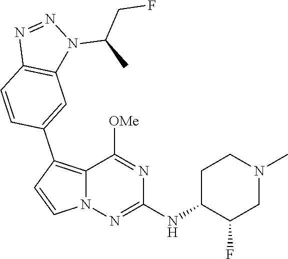 [SMILES: COc1nc(N[C@@H]2CCN(C)C[C@@H]2F)nn2ccc(-c3ccc4nnn([C@H](C)CF)c4c3)c12] |
| 1205 | 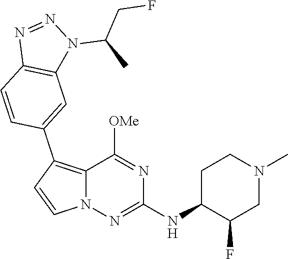 [SMILES: COc1nc(N[C@H]2CCN(C)C[C@H]2F)nn2ccc(-c3ccc4nnn([C@H](C)CF)c4c3)c12] |
| 1206 | 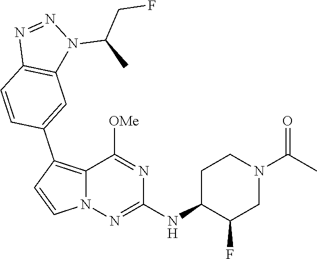 [SMILES: COc1nc(N[C@H]2CCN(C(C)=O)C[C@H]2F)nn2ccc(-c3ccc4nnn([C@H](C)CF)c4c3)c12] |
| 1207 | 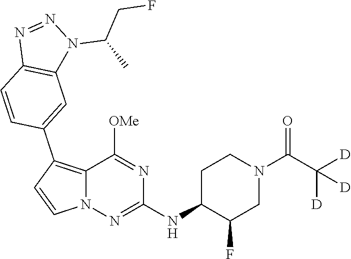 [SMILES: [2H]C([2H])([2H])C(=O)N1CC[C@H](Nc2nc(OC)c3c(-c4ccc5nnn([C@@H](C)CF)c5c4)ccn3n2)[C@H](F)C1] |
| 1208 | 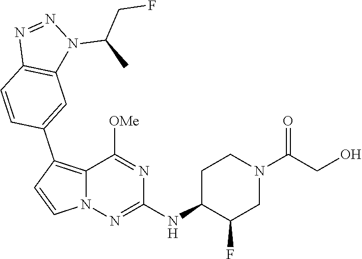 [SMILES: COc1nc(N[C@H]2CCN(C(=O)CO)C[C@H]2F)nn2ccc(-c3ccc4nnn([C@H](C)CF)c4c3)c12] |
| 1209 | 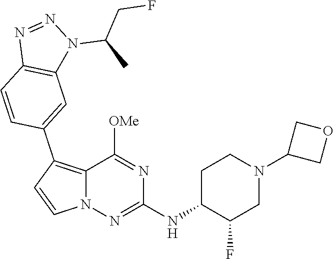 [SMILES: COc1nc(N[C@@H]2CCN(C3COC3)C[C@@H]2F)nn2ccc(-c3ccc4nnn([C@H](C)CF)c4c3)c12] |
| 1210 | 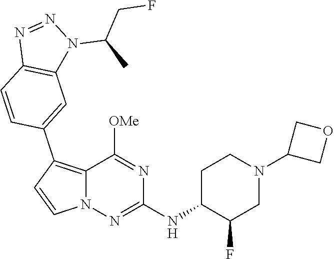 [SMILES: COc1nc(N[C@@H]2CCN(C3COC3)C[C@H]2F)nn2ccc(-c3ccc4nnn([C@H](C)CF)c4c3)c12] |
| 1211 | 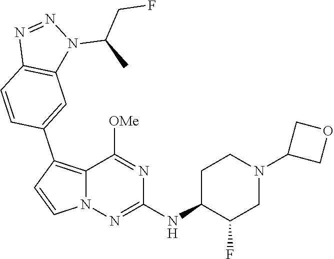 [SMILES: COc1nc(N[C@H]2CCN(C3COC3)C[C@@H]2F)nn2ccc(-c3ccc4nnn([C@H](C)CF)c4c3)c12] |
| 1212 | 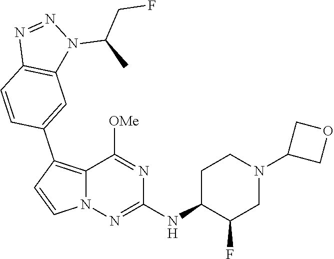 [SMILES: COc1nc(N[C@H]2CCN(C3COC3)C[C@H]2F)nn2ccc(-c3ccc4nnn([C@H](C)CF)c4c3)c12] |
| 1213 | 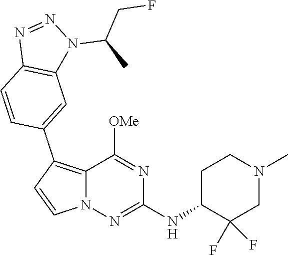 [SMILES: COc1nc(N[C@@H]2CCN(C)CC2(F)F)nn2ccc(-c3ccc4nnn([C@H](C)CF)c4c3)c12] |
| 1214 | 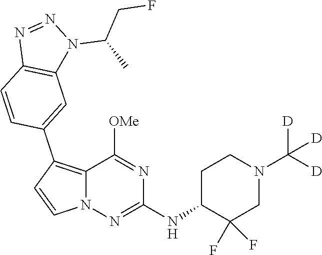 [SMILES: [2H]C([2H])([2H])N1CC[C@@H](Nc2nc(OC)c3c(-c4ccc5nnn([C@@H](C)CF)c5c4)ccn3n2)C(F)(F)C1] |
| 1215 | 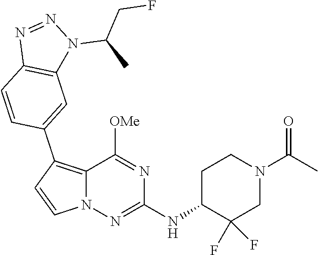 [SMILES: COc1nc(N[C@@H]2CCN(C(C)=O)CC2(F)F)nn2ccc(-c3ccc4nnn([C@H](C)CF)c4c3)c12] |
| 1216 | 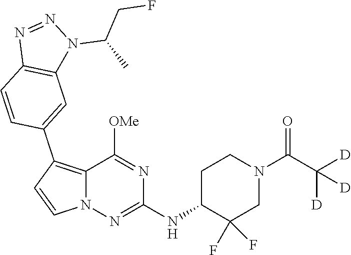 [SMILES: [2H]C([2H])([2H])C(=O)N1CC[C@@H](Nc2nc(OC)c3c(-c4ccc5nnn([C@@H](C)CF)c5c4)ccn3n2)C(F)(F)C1] |
| 1217 | 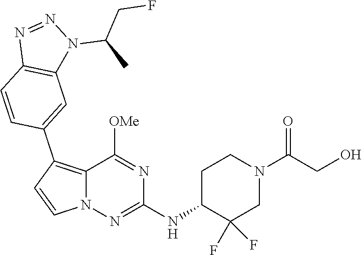 [SMILES: COc1nc(N[C@@H]2CCN(C(=O)CO)CC2(F)F)nn2ccc(-c3ccc4nnn([C@H](C)CF)c4c3)c12] |
| 1218 | 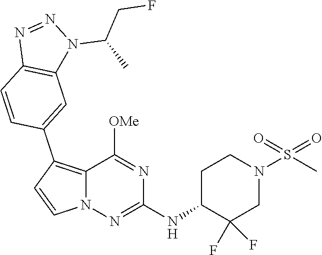 [SMILES: COc1nc(N[C@@H]2CCN(S(C)(=O)=O)CC2(F)F)nn2ccc(-c3ccc4nnn([C@@H](C)CF)c4c3)c12] |
| 1219 | 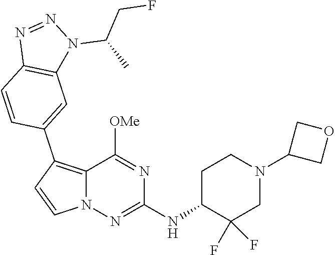 [SMILES: COc1nc(N[C@@H]2CCN(C3COC3)CC2(F)F)nn2ccc(-c3ccc4nnn([C@@H](C)CF)c4c3)c12] |
| 1220 | 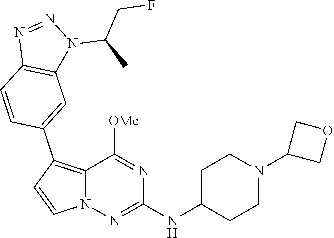 [SMILES: COc1nc(NC2CCN(C3COC3)CC2)nn2ccc(-c3ccc4nnn([C@H](C)CF)c4c3)c12] |
| 1221 | 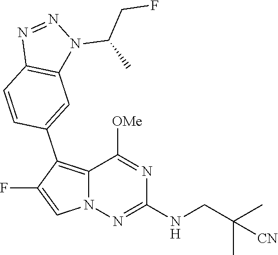 [SMILES: COc1nc(NCC(C)(C)C#N)nn2cc(F)c(-c3ccc4nnn([C@@H](C)CF)c4c3)c12] |
| 1222 | 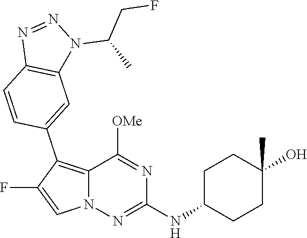 [SMILES: COc1nc(N[C@H]2CC[C@@](C)(O)CC2)nn2cc(F)c(-c3ccc4nnn([C@@H](C)CF)c4c3)c12] |
| 1223 | 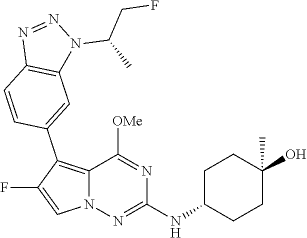 [SMILES: COc1nc(N[C@H]2CC[C@](C)(O)CC2)nn2cc(F)c(-c3ccc4nnn([C@@H](C)CF)c4c3)c12] |
| 1224 | 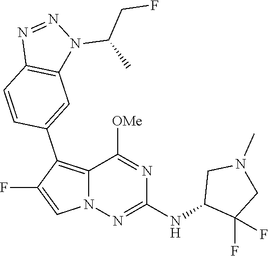 [SMILES: COc1nc(N[C@@H]2CN(C)CC2(F)F)nn2cc(F)c(-c3ccc4nnn([C@@H](C)CF)c4c3)c12] |
| 1225 | 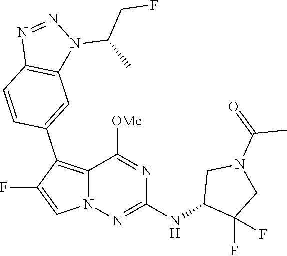 [SMILES: COc1nc(N[C@@H]2CN(C(C)=O)CC2(F)F)nn2cc(F)c(-c3ccc4nnn([C@@H](C)CF)c4c3)c12] |
| 1226 | 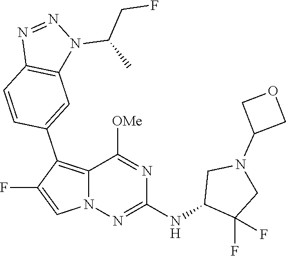 [SMILES: COc1nc(N[C@@H]2CN(C3COC3)CC2(F)F)nn2cc(F)c(-c3ccc4nnn([C@@H](C)CF)c4c3)c12] |
| 1227 | 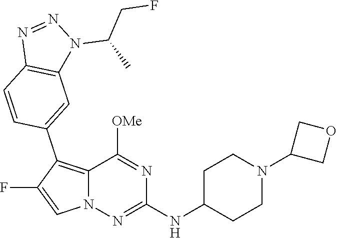 [SMILES: COc1nc(NC2CCN(C3COC3)CC2)nn2cc(F)c(-c3ccc4nnn([C@@H](C)CF)c4c3)c12] |
| 1228 | 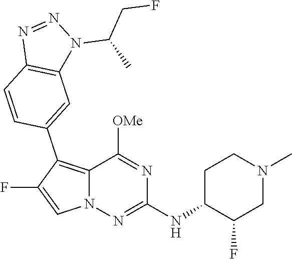 [SMILES: COc1nc(N[C@@H]2CCN(C)C[C@@H]2F)nn2cc(F)c(-c3ccc4nnn([C@@H](C)CF)c4c3)c12] |
| 1229 | 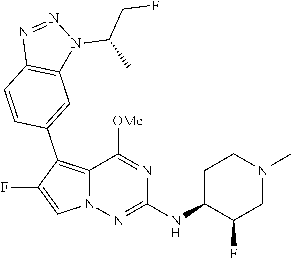 [SMILES: COc1nc(N[C@H]2CCN(C)C[C@H]2F)nn2cc(F)c(-c3ccc4nnn([C@@H](C)CF)c4c3)c12] |
| 1230 | 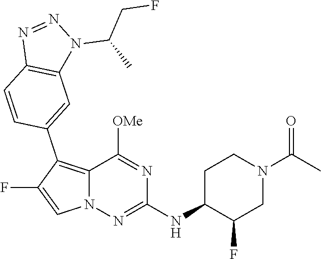 [SMILES: COc1nc(N[C@H]2CCN(C(C)=O)C[C@H]2F)nn2cc(F)c(-c3ccc4nnn([C@@H](C)CF)c4c3)c12] |
| 1231 | 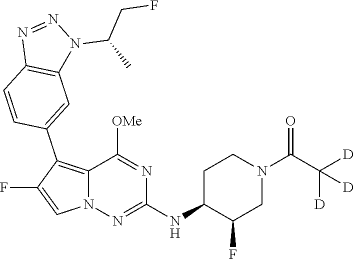 [SMILES: [2H]C([2H])([2H])C(=O)N1CC[C@H](Nc2nc(OC)c3c(-c4ccc5nnn([C@@H](C)CF)c5c4)c(F)cn3n2)[C@H](F)C1] |
| 1232 | 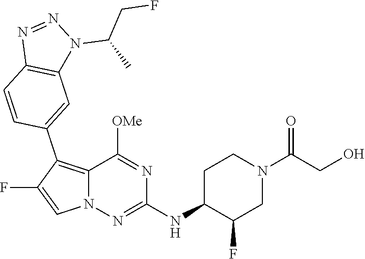 [SMILES: COc1nc(N[C@H]2CCN(C(=O)CO)C[C@H]2F)nn2cc(F)c(-c3ccc4nnn([C@@H](C)CF)c4c3)c12] |
| 1233 | 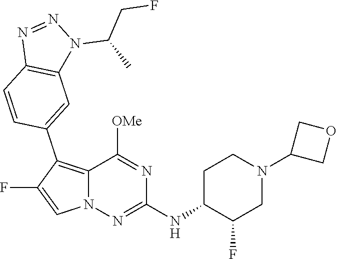 [SMILES: COc1nc(N[C@@H]2CCN(C3COC3)C[C@@H]2F)nn2cc(F)c(-c3ccc4nnn([C@@H](C)CF)c4c3)c12] |
| 1234 | 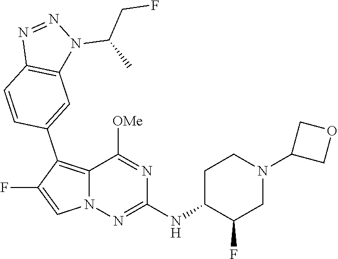 [SMILES: COc1nc(N[C@@H]2CCN(C3COC3)C[C@H]2F)nn2cc(F)c(-c3ccc4nnn([C@@H](C)CF)c4c3)c12] |
| 1235 | 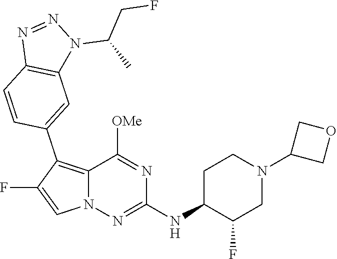 [SMILES: COc1nc(N[C@H]2CCN(C3COC3)C[C@@H]2F)nn2cc(F)c(-c3ccc4nnn([C@@H](C)CF)c4c3)c12] |
| 1236 | 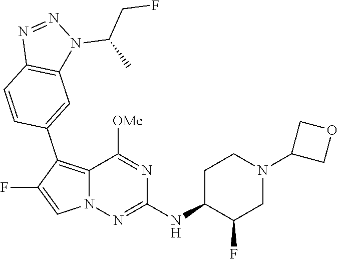 [SMILES: COc1nc(N[C@H]2CCN(C3COC3)C[C@H]2F)nn2cc(F)c(-c3ccc4nnn([C@@H](C)CF)c4c3)c12] |
| 1237 | 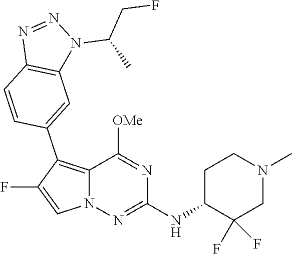 [SMILES: COc1nc(N[C@@H]2CCN(C)CC2(F)F)nn2cc(F)c(-c3ccc4nnn([C@@H](C)CF)c4c3)c12] |
| 1238 | 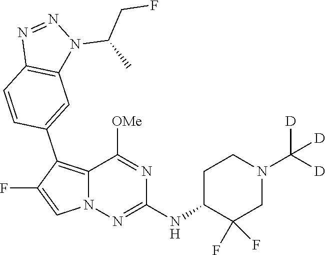 [SMILES: [2H]C([2H])([2H])N1CC[C@@H](Nc2nc(OC)c3c(-c4ccc5nnn([C@@H](C)CF)c5c4)c(F)cn3n2)C(F)(F)C1] |
| 1239 | 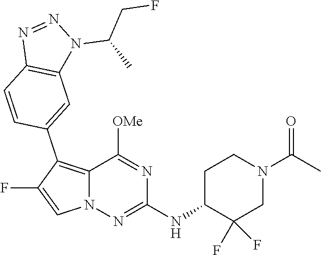 [SMILES: COc1nc(N[C@@H]2CCN(C(C)=O)CC2(F)F)nn2cc(F)c(-c3ccc4nnn([C@@H](C)CF)c4c3)c12] |
| 1240 | 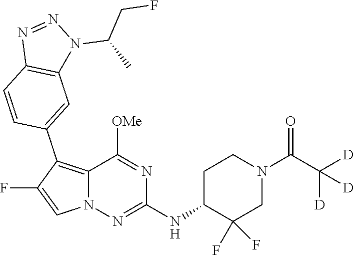 [SMILES: [2H]C([2H])([2H])C(=O)N1CC[C@@H](Nc2nc(OC)c3c(-c4ccc5nnn([C@@H](C)CF)c5c4)c(F)cn3n2)C(F)(F)C1] |
| 1241 | 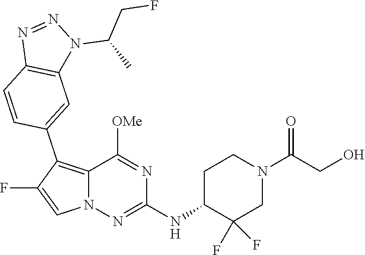 [SMILES: COc1nc(N[C@@H]2CCN(C(=O)CO)CC2(F)F)nn2cc(F)c(-c3ccc4nnn([C@@H](C)CF)c4c3)c12] |
| 1242 | 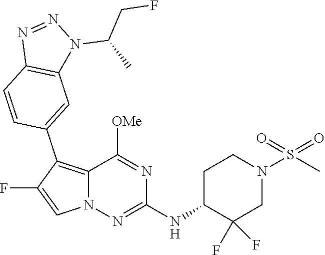 [SMILES: COc1nc(N[C@@H]2CCN(S(C)(=O)=O)CC2(F)F)nn2cc(F)c(-c3ccc4nnn([C@@H](C)CF)c4c3)c12] |
| 1243 | 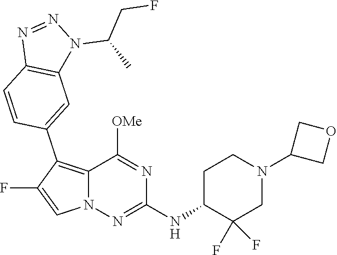 [SMILES: COc1nc(N[C@@H]2CCN(C3COC3)CC2(F)F)nn2cc(F)c(-c3ccc4nnn([C@@H](C)CF)c4c3)c12] |
| 1244 | 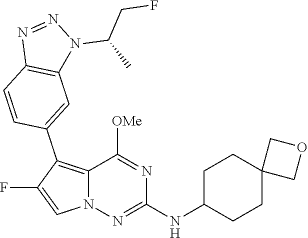 [SMILES: COc1nc(NC2CCC3(CC2)COC3)nn2cc(F)c(-c3ccc4nnn([C@@H](C)CF)c4c3)c12] |
| 1245 | 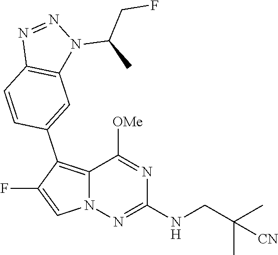 [SMILES: COc1nc(NCC(C)(C)C#N)nn2cc(F)c(-c3ccc4nnn([C@H](C)CF)c4c3)c12] |
| 1246 | 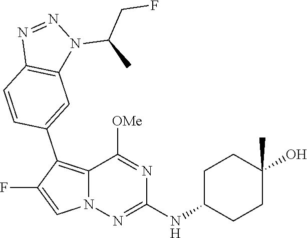 [SMILES: COc1nc(N[C@H]2CC[C@@](C)(O)CC2)nn2cc(F)c(-c3ccc4nnn([C@H](C)CF)c4c3)c12] |
| 1247 | 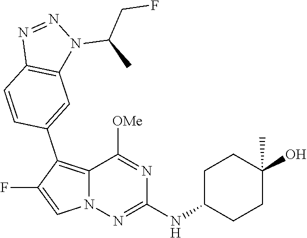 [SMILES: COc1nc(N[C@H]2CC[C@](C)(O)CC2)nn2cc(F)c(-c3ccc4nnn([C@H](C)CF)c4c3)c12] |
| 1248 | 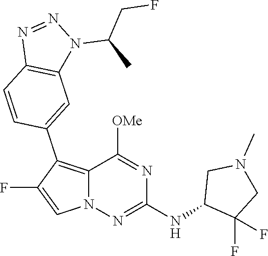 [SMILES: COc1nc(N[C@@H]2CN(C)CC2(F)F)nn2cc(F)c(-c3ccc4nnn([C@H](C)CF)c4c3)c12] |
| 1249 | 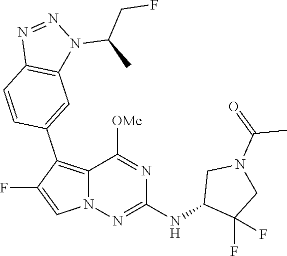 [SMILES: COc1nc(N[C@@H]2CN(C(C)=O)CC2(F)F)nn2cc(F)c(-c3ccc4nnn([C@H](C)CF)c4c3)c12] |
| 1250 | 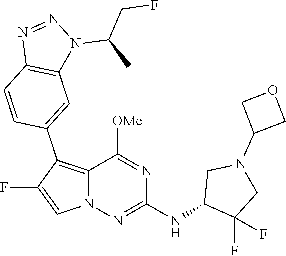 [SMILES: COc1nc(N[C@@H]2CN(C3COC3)CC2(F)F)nn2cc(F)c(-c3ccc4nnn([C@H](C)CF)c4c3)c12] |
| 1251 | 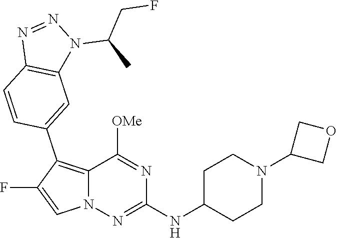 [SMILES: COc1nc(NC2CCN(C3COC3)CC2)nn2cc(F)c(-c3ccc4nnn([C@H](C)CF)c4c3)c12] |
| 1252 | 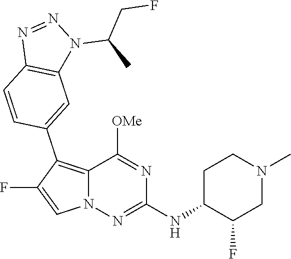 [SMILES: COc1nc(N[C@@H]2CCN(C)C[C@@H]2F)nn2cc(F)c(-c3ccc4nnn([C@H](C)CF)c4c3)c12] |
| 1253 | 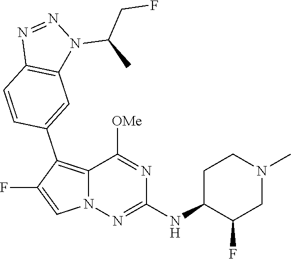 [SMILES: COc1nc(N[C@H]2CCN(C)C[C@H]2F)nn2cc(F)c(-c3ccc4nnn([C@H](C)CF)c4c3)c12] |
| 1254 | 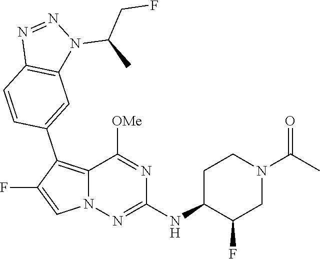 [SMILES: COc1nc(N[C@H]2CCN(C(C)=O)C[C@H]2F)nn2cc(F)c(-c3ccc4nnn([C@H](C)CF)c4c3)c12] |
| 1255 | 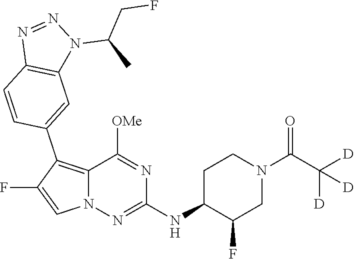 [SMILES: [2H]C([2H])([2H])C(=O)N1CC[C@H](Nc2nc(OC)c3c(-c4ccc5nnn([C@H](C)CF)c5c4)c(F)cn3n2)[C@H](F)C1] |
| 1256 | 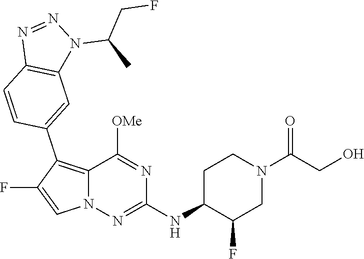 [SMILES: COc1nc(N[C@H]2CCN(C(=O)CO)C[C@H]2F)nn2cc(F)c(-c3ccc4nnn([C@H](C)CF)c4c3)c12] |
| 1257 | 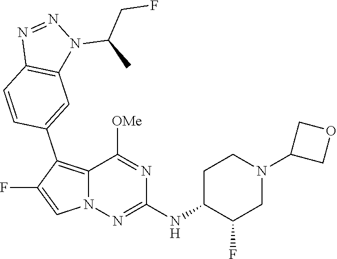 [SMILES: COc1nc(N[C@@H]2CCN(C3COC3)C[C@@H]2F)nn2cc(F)c(-c3ccc4nnn([C@H](C)CF)c4c3)c12] |
| 1258 | 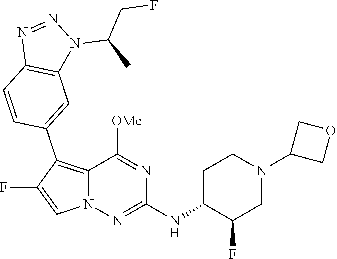 [SMILES: COc1nc(N[C@@H]2CCN(C3COC3)C[C@H]2F)nn2cc(F)c(-c3ccc4nnn([C@H](C)CF)c4c3)c12] |
| 1259 | 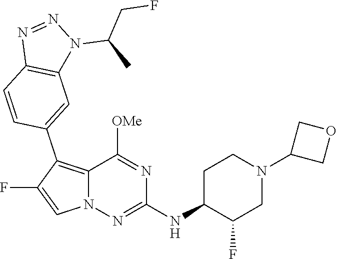 [SMILES: COc1nc(N[C@H]2CCN(C3COC3)C[C@@H]2F)nn2cc(F)c(-c3ccc4nnn([C@H](C)CF)c4c3)c12] |
| 1260 | 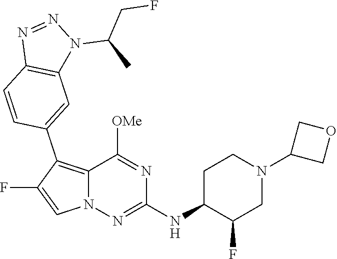 [SMILES: COc1nc(N[C@H]2CCN(C3COC3)C[C@H]2F)nn2cc(F)c(-c3ccc4nnn([C@H](C)CF)c4c3)c12] |
| 1261 | 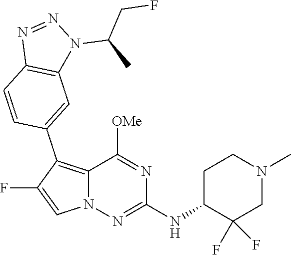 [SMILES: COc1nc(N[C@@H]2CCN(C)CC2(F)F)nn2cc(F)c(-c3ccc4nnn([C@H](C)CF)c4c3)c12] |
| 1262 | 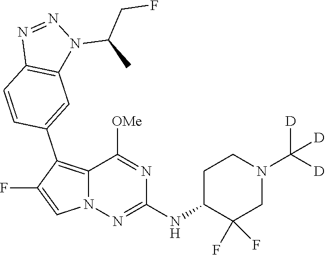 [SMILES: [2H]C([2H])([2H])N1CC[C@@H](Nc2nc(OC)c3c(-c4ccc5nnn([C@H](C)CF)c5c4)c(F)cn3n2)C(F)(F)C1] |
| 1263 | 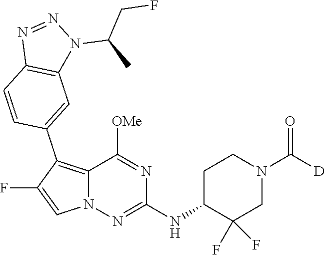 [SMILES: [2H]C(=O)N1CC[C@@H](Nc2nc(OC)c3c(-c4ccc5nnn([C@H](C)CF)c5c4)c(F)cn3n2)C(F)(F)C1] |
| 1264 | 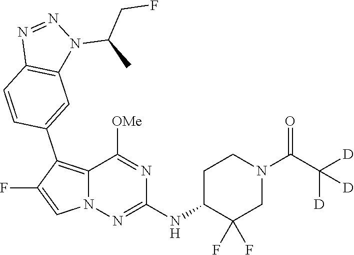 [SMILES: [2H]C([2H])([2H])C(=O)N1CC[C@@H](Nc2nc(OC)c3c(-c4ccc5nnn([C@H](C)CF)c5c4)c(F)cn3n2)C(F)(F)C1] |
| 1265 | 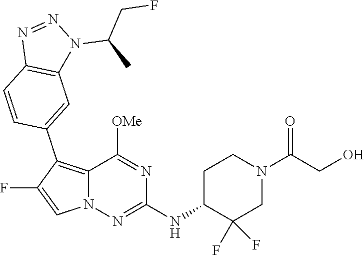 [SMILES: COc1nc(N[C@@H]2CCN(C(=O)CO)CC2(F)F)nn2cc(F)c(-c3ccc4nnn([C@H](C)CF)c4c3)c12] |
| 1266 | 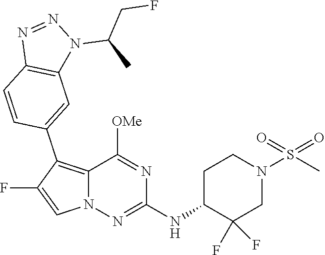 [SMILES: COc1nc(N[C@@H]2CCN(S(C)(=O)=O)CC2(F)F)nn2cc(F)c(-c3ccc4nnn([C@H](C)CF)c4c3)c12] |
| 1267 | 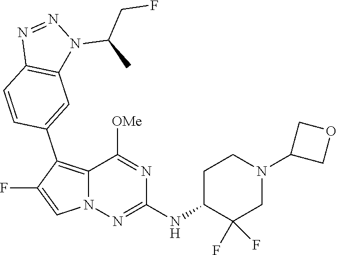 [SMILES: COc1nc(N[C@@H]2CCN(C3COC3)CC2(F)F)nn2cc(F)c(-c3ccc4nnn([C@H](C)CF)c4c3)c12] |
| 1268 | 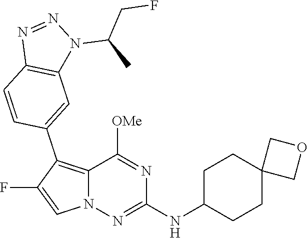 [SMILES: COc1nc(NC2CCC3(CC2)COC3)nn2cc(F)c(-c3ccc4nnn([C@H](C)CF)c4c3)c12] |
| 1269 | 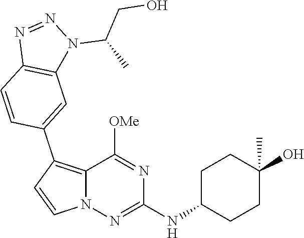 [SMILES: COc1nc(N[C@H]2CC[C@](C)(O)CC2)nn2ccc(-c3ccc4nnn([C@@H](C)CO)c4c3)c12] |
| 1270 | 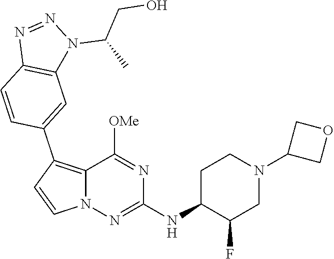 [SMILES: COc1nc(N[C@H]2CCN(C3COC3)C[C@H]2F)nn2ccc(-c3ccc4nnn([C@@H](C)CO)c4c3)c12] |
| 1271 | 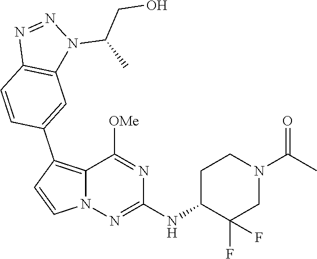 [SMILES: COc1nc(N[C@@H]2CCN(C(C)=O)CC2(F)F)nn2ccc(-c3ccc4nnn([C@@H](C)CO)c4c3)c12] |
| 1272 | 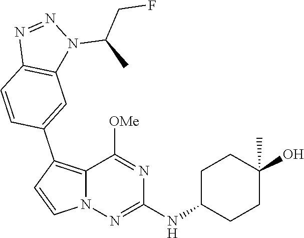 [SMILES: COc1nc(N[C@H]2CC[C@](C)(O)CC2)nn2ccc(-c3ccc4nnn([C@H](C)CF)c4c3)c12] |
| 1273 | 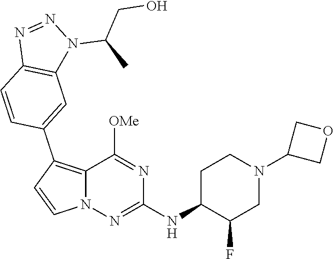 [SMILES: COc1nc(N[C@H]2CCN(C3COC3)C[C@H]2F)nn2ccc(-c3ccc4nnn([C@H](C)CO)c4c3)c12] |
| 1274 | 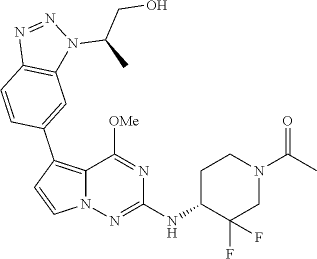 [SMILES: COc1nc(N[C@@H]2CCN(C(C)=O)CC2(F)F)nn2ccc(-c3ccc4nnn([C@H](C)CO)c4c3)c12] |
| 1275 | 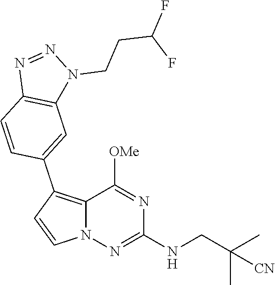 [SMILES: COc1nc(NCC(C)(C)C#N)nn2ccc(-c3ccc4nnn(CCC(F)F)c4c3)c12] |
| 1276 | 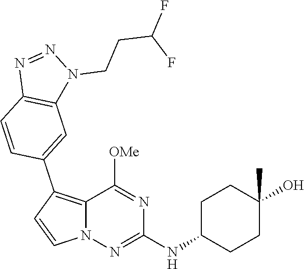 [SMILES: COc1nc(N[C@H]2CC[C@@](C)(O)CC2)nn2ccc(-c3ccc4nnn(CCC(F)F)c4c3)c12] |
| 1277 | 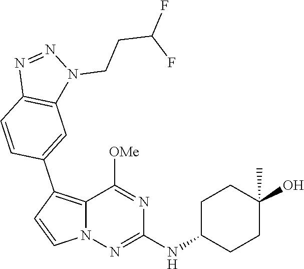 [SMILES: COc1nc(N[C@H]2CC[C@](C)(O)CC2)nn2ccc(-c3ccc4nnn(CCC(F)F)c4c3)c12] |
| 1278 | 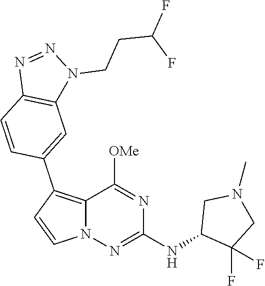 |
| 1279 | 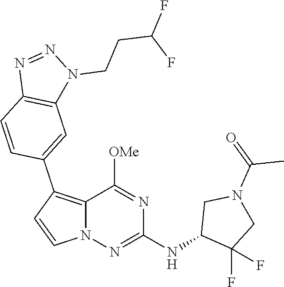 [SMILES: COc1nc(N[C@@H]2CN(C(C)=O)CC2(F)F)nn2ccc(-c3ccc4nnn(CCC(F)F)c4c3)c12] |
| 1280 | 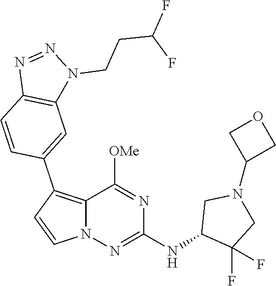 |
| 1281 | 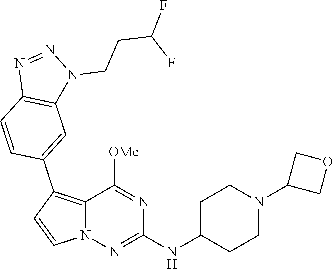 [SMILES: COc1nc(NC2CCN(C3COC3)CC2)nn2ccc(-c3ccc4nnn(CCC(F)F)c4c3)c12] |
| 1282 | 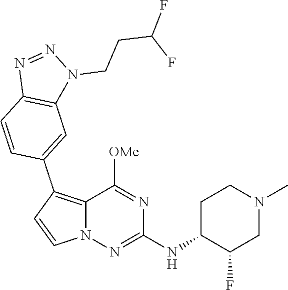 [SMILES: COc1nc(N[C@@H]2CCN(C)C[C@@H]2F)nn2ccc(-c3ccc4nnn(CCC(F)F)c4c3)c12] |
| 1283 | 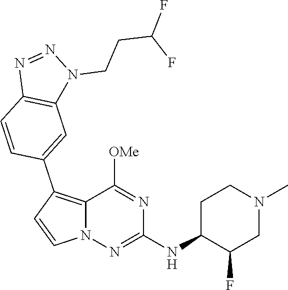 [SMILES: COc1nc(N[C@H]2CCN(C)C[C@H]2F)nn2ccc(-c3ccc4nnn(CCC(F)F)c4c3)c12] |
| 1284 | 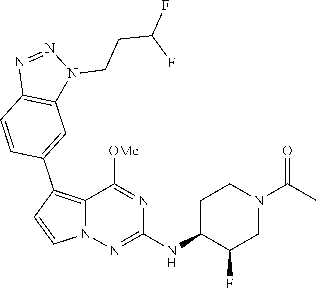 |
| 1285 | 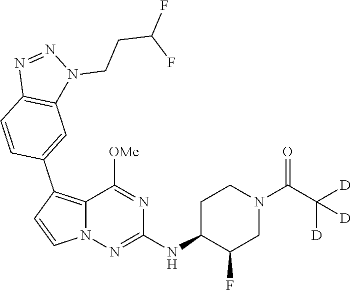 [SMILES: [2H]C([2H])([2H])C(=O)N1CC[C@H](Nc2nc(OC)c3c(-c4ccc5nnn(CCC(F)F)c5c4)ccn3n2)[C@H](F)C1] |
| 1286 | 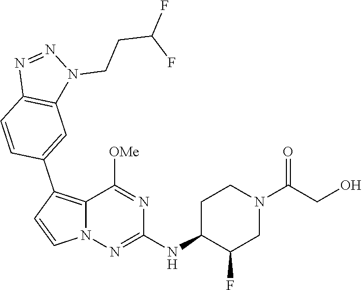 [SMILES: COc1nc(N[C@H]2CCN(C(=O)CO)C[C@H]2F)nn2ccc(-c3ccc4nnn(CCC(F)F)c4c3)c12] |
| 1287 | 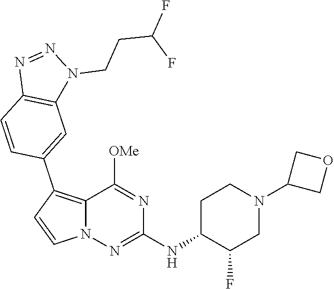 |
| 1288 | 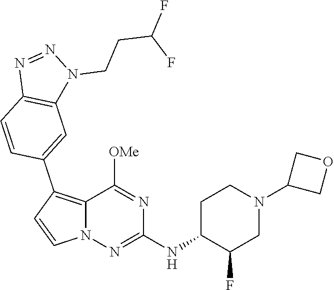 [SMILES: COc1nc(N[C@@H]2CCN(C3COC3)C[C@H]2F)nn2ccc(-c3ccc4nnn(CCC(F)F)c4c3)c12] |
| 1289 | 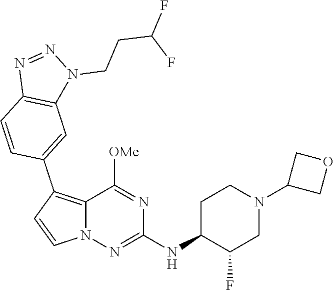 [SMILES: COc1nc(N[C@H]2CCN(C3COC3)C[C@@H]2F)nn2ccc(-c3ccc4nnn(CCC(F)F)c4c3)c12] |
| 1290 | 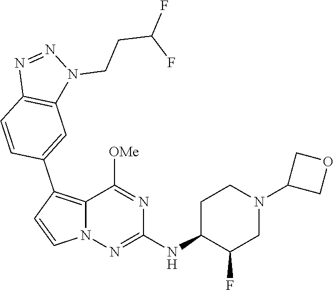 [SMILES: COc1nc(N[C@H]2CCN(C3COC3)C[C@H]2F)nn2ccc(-c3ccc4nnn(CCC(F)F)c4c3)c12] |
| 1291 | 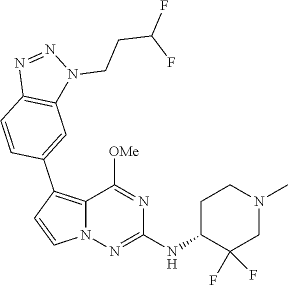 [SMILES: COc1nc(N[C@@H]2CCN(C)CC2(F)F)nn2ccc(-c3ccc4nnn(CCC(F)F)c4c3)c12] |
| 1292 | 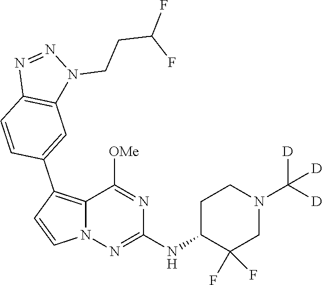 [SMILES: [2H]C([2H])([2H])N1CC[C@@H](Nc2nc(OC)c3c(-c4ccc5nnn(CCC(F)F)c5c4)ccn3n2)C(F)(F)C1] |
| 1293 | 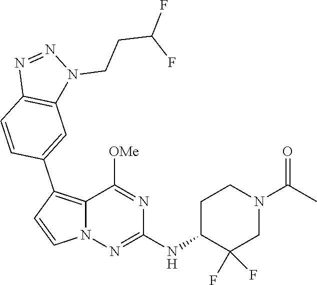 [SMILES: COc1nc(N[C@@H]2CCN(C(C)=O)CC2(F)F)nn2ccc(-c3ccc4nnn(CCC(F)F)c4c3)c12] |
| 1294 | 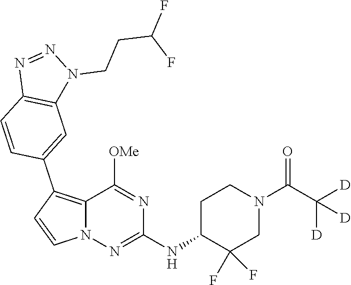 [SMILES: [2H]C([2H])([2H])C(=O)N1CC[C@@H](Nc2nc(OC)c3c(-c4ccc5nnn(CCC(F)F)c5c4)ccn3n2)C(F)(F)C1] |
| 1295 | 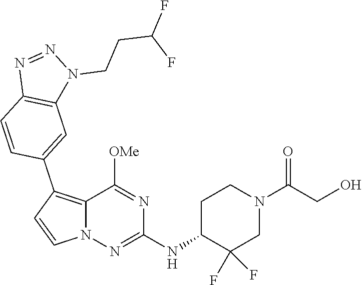 [SMILES: COc1nc(N[C@@H]2CCN(C(=O)CO)CC2(F)F)nn2ccc(-c3ccc4nnn(CCC(F)F)c4c3)c12] |
| 1296 | 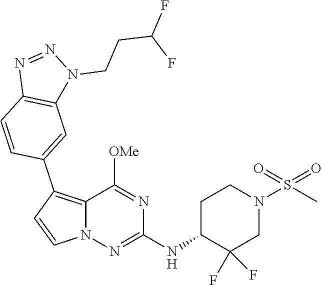 [SMILES: COc1nc(N[C@@H]2CCN(S(C)(=O)=O)CC2(F)F)nn2ccc(-c3ccc4nnn(CCC(F)F)c4c3)c12] |
| 1297 | 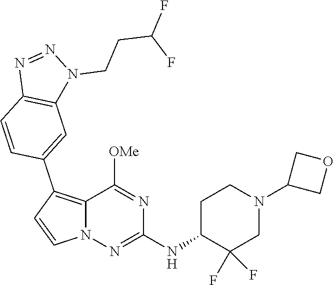 [SMILES: COc1nc(N[C@@H]2CCN(C3COC3)CC2(F)F)nn2ccc(-c3ccc4nnn(CCC(F)F)c4c3)c12] |
| 1298 | 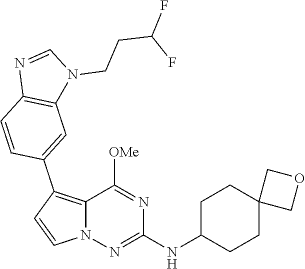 [SMILES: COc1nc(NC2CCC3(CC2)COC3)nn2ccc(-c3ccc4ncn(CCC(F)F)c4c3)c12] |
| 1299 | 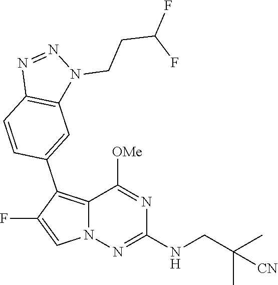 [SMILES: COc1nc(NCC(C)(C)C#N)nn2cc(F)c(-c3ccc4nnn(CCC(F)F)c4c3)c12] |
| 1300 | 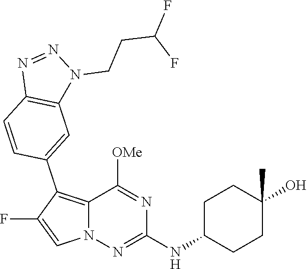 [SMILES: COc1nc(N[C@H]2CC[C@@](C)(O)CC2)nn2cc(F)c(-c3ccc4nnn(CCC(F)F)c4c3)c12] |
| 1301 | 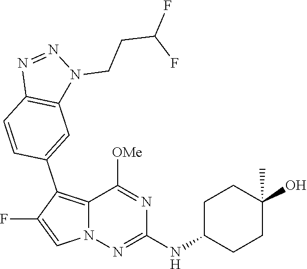 [SMILES: COc1nc(N[C@H]2CC[C@](C)(O)CC2)nn2cc(F)c(-c3ccc4nnn(CCC(F)F)c4c3)c12] |
| 1302 | 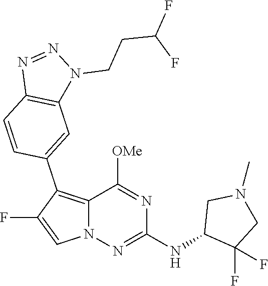 |
| 1303 | 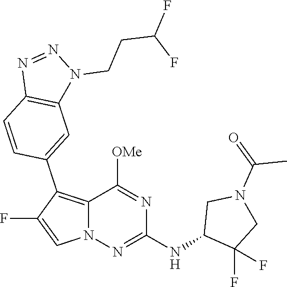 [SMILES: COc1nc(N[C@@H]2CN(C(C)=O)CC2(F)F)nn2cc(F)c(-c3ccc4nnn(CCC(F)F)c4c3)c12] |
| 1304 | 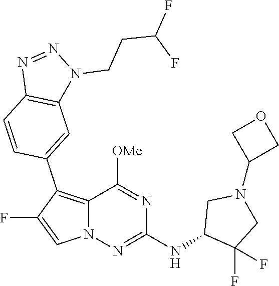 |
| 1305 | 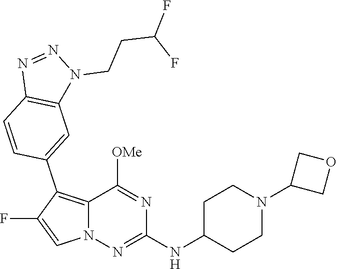 [SMILES: COc1nc(NC2CCN(C3COC3)CC2)nn2cc(F)c(-c3ccc4nnn(CCC(F)F)c4c3)c12] |
| 1306 | 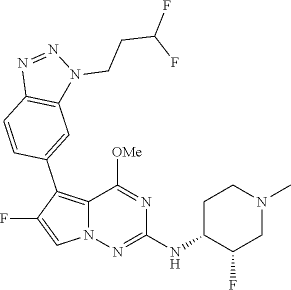 [SMILES: COc1nc(N[C@@H]2CCN(C)C[C@@H]2F)nn2cc(F)c(-c3ccc4nnn(CCC(F)F)c4c3)c12] |
| 1307 | 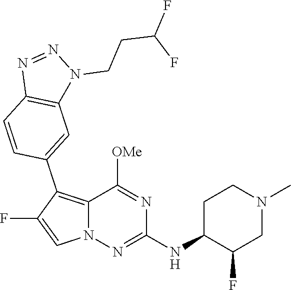 [SMILES: COc1nc(N[C@H]2CCN(C)C[C@H]2F)nn2cc(F)c(-c3ccc4nnn(CCC(F)F)c4c3)c12] |
| 1308 | 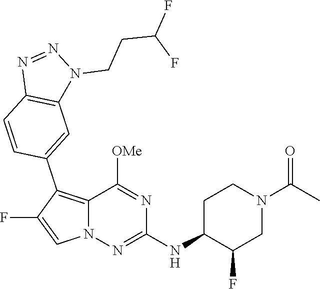 |
| 1309 | 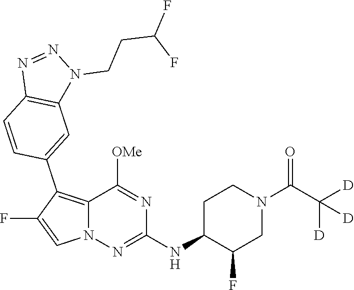 [SMILES: [2H]C([2H])([2H])C(=O)N1CC[C@H](Nc2nc(OC)c3c(-c4ccc5nnn(CCC(F)F)c5c4)c(F)cn3n2)[C@H](F)C1] |
| 1310 | 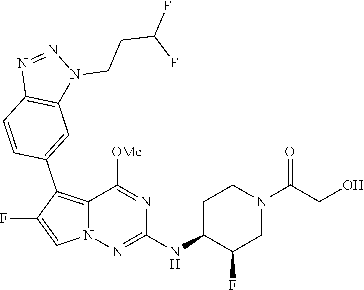 [SMILES: COc1nc(N[C@H]2CCN(C(=O)CO)C[C@H]2F)nn2cc(F)c(-c3ccc4nnn(CCC(F)F)c4c3)c12] |
| 1311 | 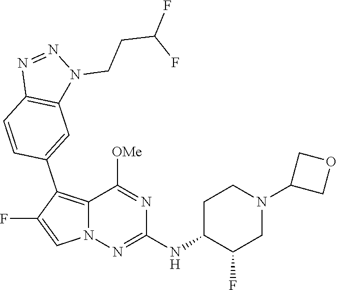 [SMILES: COc1nc(N[C@@H]2CCN(C3COC3)C[C@@H]2F)nn2cc(F)c(-c3ccc4nnn(CCC(F)F)c4c3)c12] |
| 1312 | 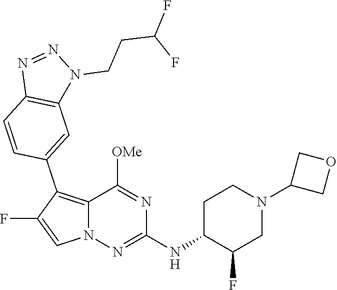 [SMILES: COc1nc(N[C@@H]2CCN(C3COC3)C[C@H]2F)nn2cc(F)c(-c3ccc4nnn(CCC(F)F)c4c3)c12] |
| 1313 | 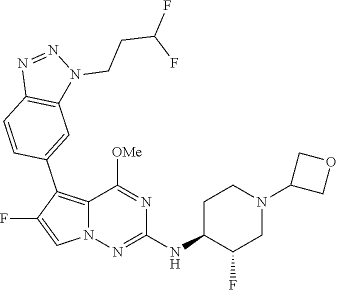 [SMILES: COc1nc(N[C@H]2CCN(C3COC3)C[C@@H]2F)nn2cc(F)c(-c3ccc4nnn(CCC(F)F)c4c3)c12] |
| 1314 | 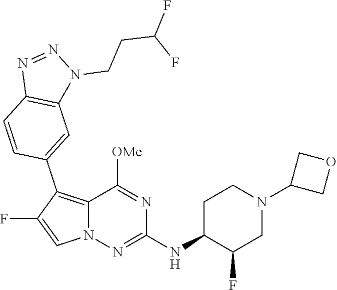 [SMILES: COc1nc(N[C@H]2CCN(C3COC3)C[C@H]2F)nn2cc(F)c(-c3ccc4nnn(CCC(F)F)c4c3)c12] |
| 1315 | 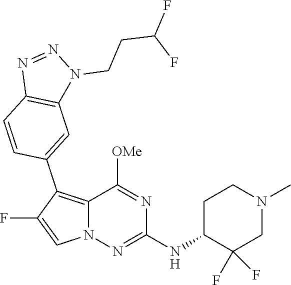 [SMILES: COc1nc(N[C@@H]2CCN(C)CC2(F)F)nn2cc(F)c(-c3ccc4nnn(CCC(F)F)c4c3)c12] |
| 1316 | 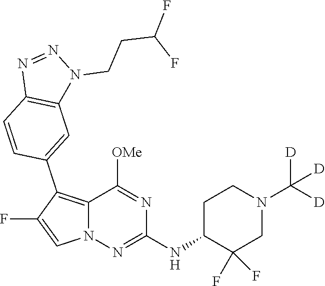 [SMILES: [2H]C([2H])([2H])N1CC[C@@H](Nc2nc(OC)c3c(-c4ccc5nnn(CCC(F)F)c5c4)c(F)cn3n2)C(F)(F)C1] |
| 1317 | 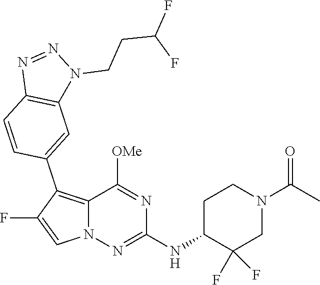 |
| 1318 | 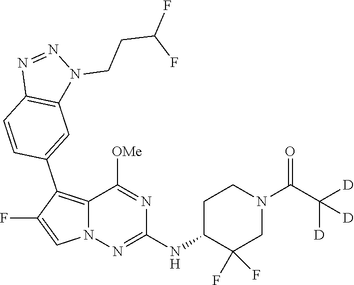 [SMILES: [2H]C([2H])([2H])C(=O)N1CC[C@@H](Nc2nc(OC)c3c(-c4ccc5nnn(CCC(F)F)c5c4)c(F)cn3n2)C(F)(F)C1] |
| 1319 | 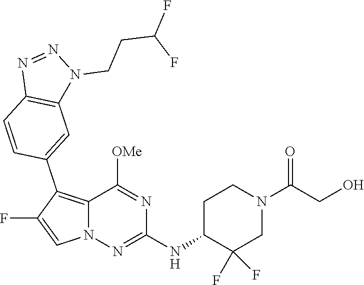 [SMILES: COc1nc(N[C@@H]2CCN(C(=O)CO)CC2(F)F)nn2cc(F)c(-c3ccc4nnn(CCC(F)F)c4c3)c12] |
| 1320 | 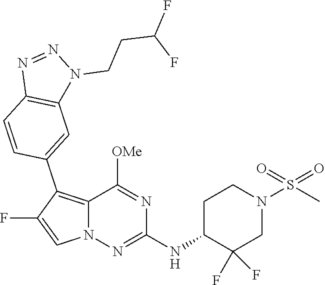 [SMILES: COc1nc(N[C@@H]2CCN(S(C)(=O)=O)CC2(F)F)nn2cc(F)c(-c3ccc4nnn(CCC(F)F)c4c3)c12] |
| 1321 | 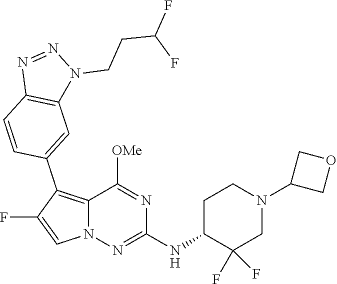 |
| 1322 | 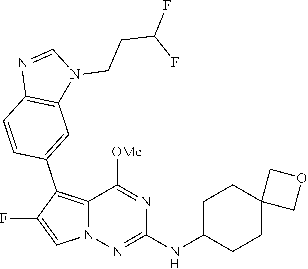 [SMILES: COc1nc(NC2CCC3(CC2)COC3)nn2cc(F)c(-c3ccc4ncn(CCC(F)F)c4c3)c12] |
| 1323 | 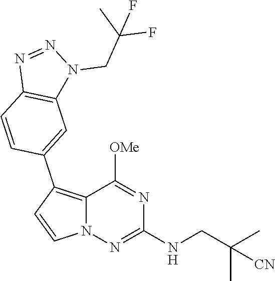 [SMILES: COc1nc(NCC(C)(C)C#N)nn2ccc(-c3ccc4nnn(CC(C)(F)F)c4c3)c12] |
| 1324 | 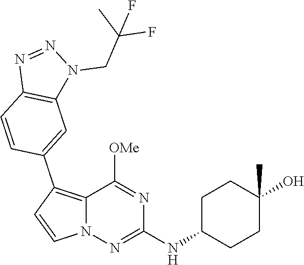 [SMILES: COc1nc(N[C@H]2CC[C@@](C)(O)CC2)nn2ccc(-c3ccc4nnn(CC(C)(F)F)c4c3)c12] |
| 1325 | 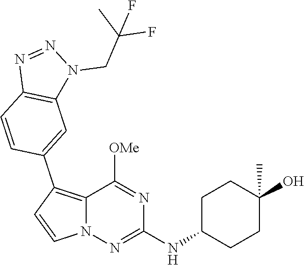 [SMILES: COc1nc(N[C@H]2CC[C@](C)(O)CC2)nn2ccc(-c3ccc4nnn(CC(C)(F)F)c4c3)c12] |
| 1326 | 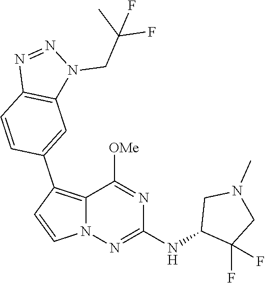 [SMILES: COc1nc(N[C@@H]2CN(C)CC2(F)F)nn2ccc(-c3ccc4nnn(CC(C)(F)F)c4c3)c12] |
| 1327 | 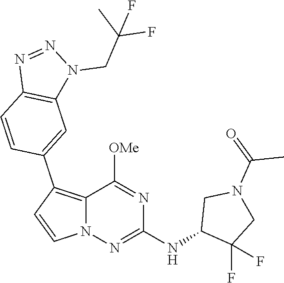 [SMILES: COc1nc(N[C@@H]2CN(C(C)=O)CC2(F)F)nn2ccc(-c3ccc4nnn(CC(C)(F)F)c4c3)c12] |
| 1328 | 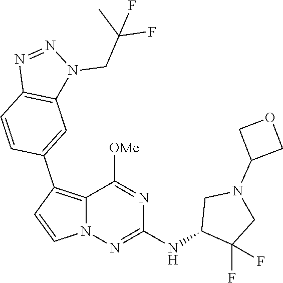 [SMILES: COc1nc(N[C@@H]2CN(C3COC3)CC2(F)F)nn2ccc(-c3ccc4nnn(CC(C)(F)F)c4c3)c12] |
| 1329 | 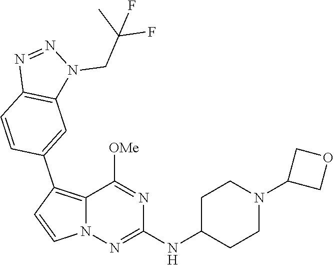 [SMILES: COc1nc(NC2CCN(C3COC3)CC2)nn2ccc(-c3ccc4nnn(CC(C)(F)F)c4c3)c12] |
| 1330 | 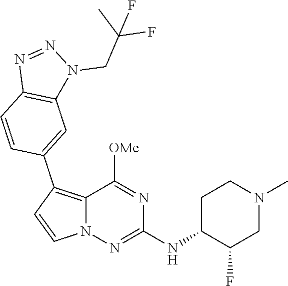 [SMILES: COc1nc(N[C@@H]2CCN(C)C[C@@H]2F)nn2ccc(-c3ccc4nnn(CC(C)(F)F)c4c3)c12] |
| 1331 | 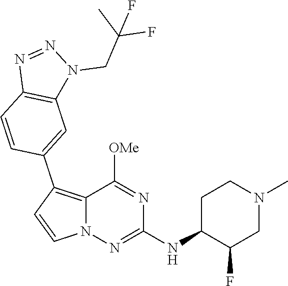 [SMILES: COc1nc(N[C@H]2CCN(C)C[C@H]2F)nn2ccc(-c3ccc4nnn(CC(C)(F)F)c4c3)c12] |
| 1332 | 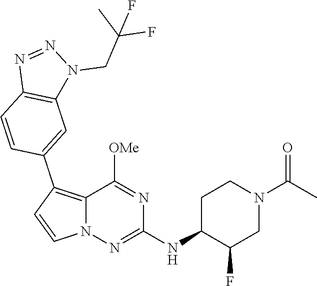 [SMILES: COc1nc(N[C@H]2CCN(C(C)=O)C[C@H]2F)nn2ccc(-c3ccc4nnn(CC(C)(F)F)c4c3)c12] |
| 1333 | 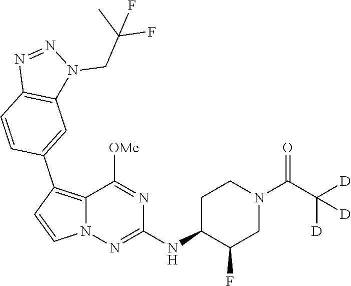 [SMILES: [2H]C([2H])([2H])C(=O)N1CC[C@H](Nc2nc(OC)c3c(-c4ccc5nnn(CC(C)(F)F)c5c4)ccn3n2)[C@H](F)C1] |
| 1334 | 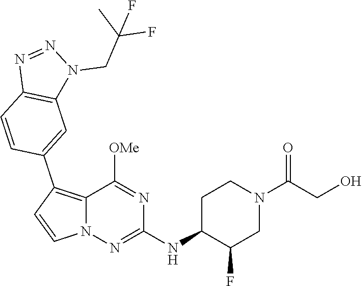 [SMILES: COc1nc(N[C@H]2CCN(C(=O)CO)C[C@H]2F)nn2ccc(-c3ccc4nnn(CC(C)(F)F)c4c3)c12] |
| 1335 | 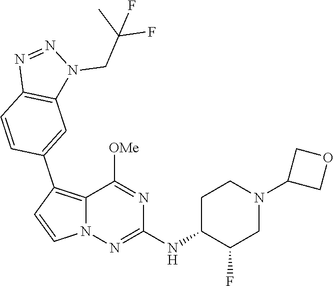 [SMILES: COc1nc(N[C@@H]2CCN(C3COC3)C[C@@H]2F)nn2ccc(-c3ccc4nnn(CC(C)(F)F)c4c3)c12] |
| 1336 | 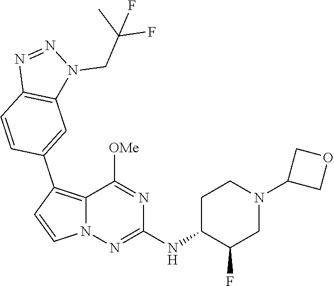 [SMILES: COc1nc(N[C@@H]2CCN(C3COC3)C[C@H]2F)nn2ccc(-c3ccc4nnn(CC(C)(F)F)c4c3)c12] |
| 1337 | 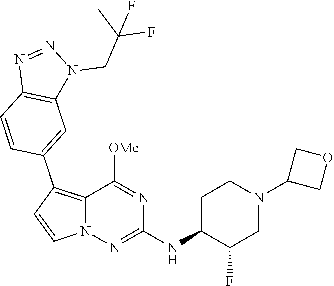 [SMILES: COc1nc(N[C@H]2CCN(C3COC3)C[C@@H]2F)nn2ccc(-c3ccc4nnn(CC(C)(F)F)c4c3)c12] |
| 1338 | 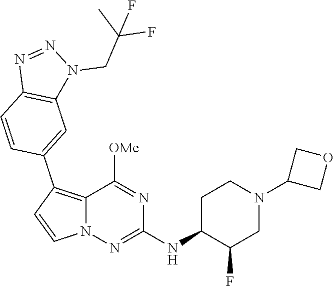 [SMILES: COc1nc(N[C@H]2CCN(C3COC3)C[C@H]2F)nn2ccc(-c3ccc4nnn(CC(C)(F)F)c4c3)c12] |
| 1339 | 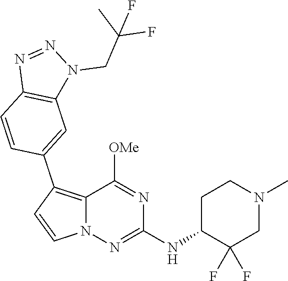 [SMILES: COc1nc(N[C@@H]2CCN(C)CC2(F)F)nn2ccc(-c3ccc4nnn(CC(C)(F)F)c4c3)c12] |
| 1340 | 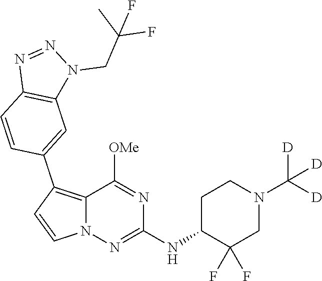 [SMILES: [2H]C([2H])([2H])N1CC[C@@H](Nc2nc(OC)c3c(-c4ccc5nnn(CC(C)(F)F)c5c4)ccn3n2)C(F)(F)C1] |
| 1341 | 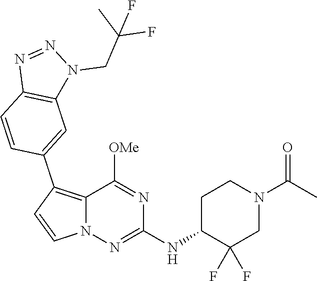 [SMILES: COc1nc(N[C@@H]2CCN(C(C)=O)CC2(F)F)nn2ccc(-c3ccc4nnn(CC(C)(F)F)c4c3)c12] |
| 1342 | 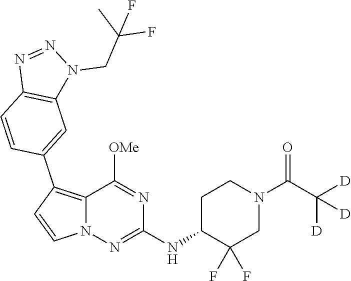 [SMILES: [2H]C([2H])([2H])C(=O)N1CC[C@@H](Nc2nc(OC)c3c(-c4ccc5nnn(CC(C)(F)F)c5c4)ccn3n2)C(F)(F)C1] |
| 1343 | 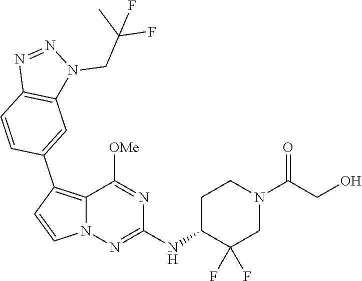 [SMILES: COc1nc(N[C@@H]2CCN(C(=O)CO)CC2(F)F)nn2ccc(-c3ccc4nnn(CC(C)(F)F)c4c3)c12] |
| 1344 | 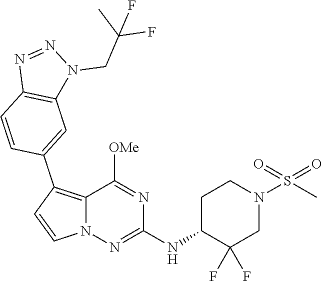 [SMILES: COc1nc(N[C@@H]2CCN(S(C)(=O)=O)CC2(F)F)nn2ccc(-c3ccc4nnn(CC(C)(F)F)c4c3)c12] |
| 1345 | 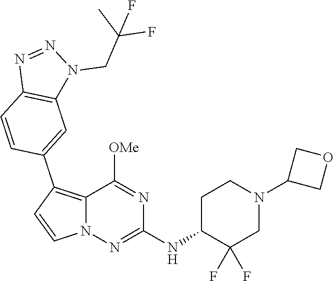 [SMILES: COc1nc(N[C@@H]2CCN(C3COC3)CC2(F)F)nn2ccc(-c3ccc4nnn(CC(C)(F)F)c4c3)c12] |
| 1346 | 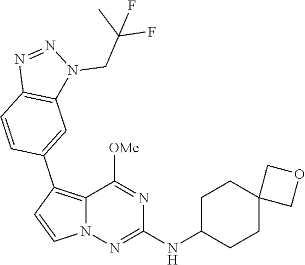 [SMILES: COc1nc(NC2CCC3(CC2)COC3)nn2ccc(-c3ccc4nnn(CC(C)(F)F)c4c3)c12] |
| 1347 | 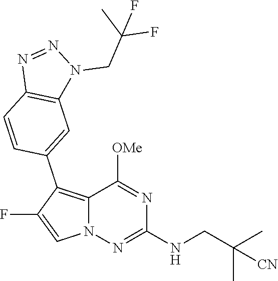 [SMILES: COc1nc(NCC(C)(C)C#N)nn2cc(F)c(-c3ccc4nnn(CC(C)(F)F)c4c3)c12] |
| 1348 | 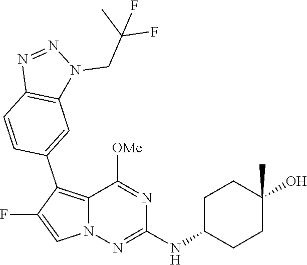 [SMILES: COc1nc(N[C@H]2CC[C@@](C)(O)CC2)nn2cc(F)c(-c3ccc4nnn(CC(C)(F)F)c4c3)c12] |
| 1349 | 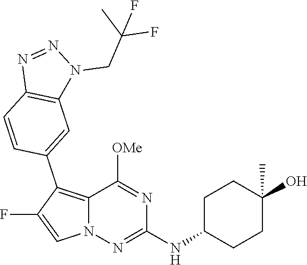 [SMILES: COc1nc(N[C@H]2CC[C@](C)(O)CC2)nn2cc(F)c(-c3ccc4nnn(CC(C)(F)F)c4c3)c12] |
| 1350 | 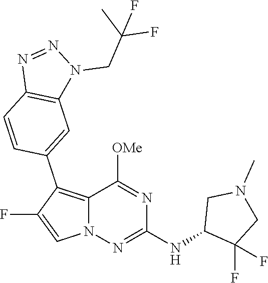 [SMILES: COc1nc(N[C@@H]2CN(C)CC2(F)F)nn2cc(F)c(-c3ccc4nnn(CC(C)(F)F)c4c3)c12] |
| 1351 | 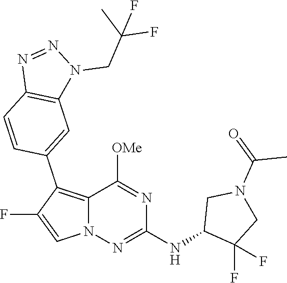 [SMILES: COc1nc(N[C@@H]2CN(C(C)=O)CC2(F)F)nn2cc(F)c(-c3ccc4nnn(CC(C)(F)F)c4c3)c12] |
| 1352 | 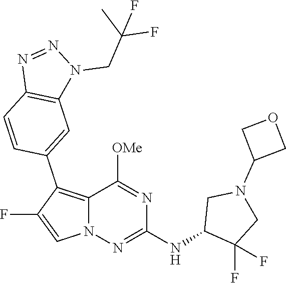 [SMILES: COc1nc(N[C@@H]2CN(C3COC3)CC2(F)F)nn2cc(F)c(-c3ccc4nnn(CC(C)(F)F)c4c3)c12] |
| 1353 | 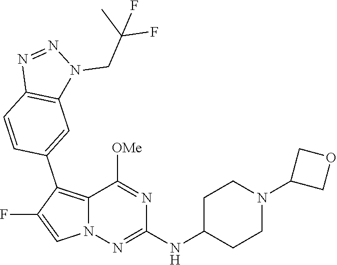 [SMILES: COc1nc(NC2CCN(C3COC3)CC2)nn2cc(F)c(-c3ccc4nnn(CC(C)(F)F)c4c3)c12] |
| 1354 | 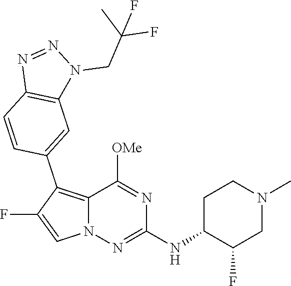 [SMILES: COc1nc(N[C@@H]2CCN(C)C[C@@H]2F)nn2cc(F)c(-c3ccc4nnn(CC(C)(F)F)c4c3)c12] |
| 1355 | 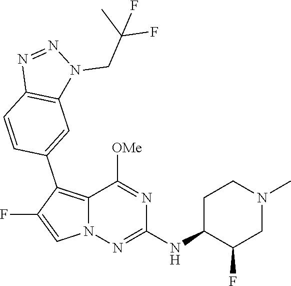 [SMILES: COc1nc(N[C@H]2CCN(C)C[C@H]2F)nn2cc(F)c(-c3ccc4nnn(CC(C)(F)F)c4c3)c12] |
| 1356 | 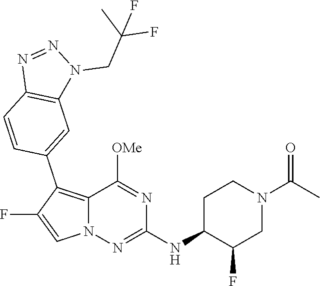 [SMILES: COc1nc(N[C@H]2CCN(C(C)=O)C[C@H]2F)nn2cc(F)c(-c3ccc4nnn(CC(C)(F)F)c4c3)c12] |
| 1357 | 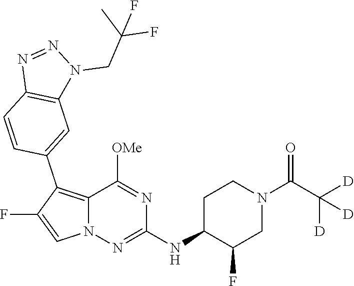 [SMILES: [2H]C([2H])([2H])C(=O)N1CC[C@H](Nc2nc(OC)c3c(-c4ccc5nnn(CC(C)(F)F)c5c4)c(F)cn3n2)[C@H](F)C1] |
| 1358 | 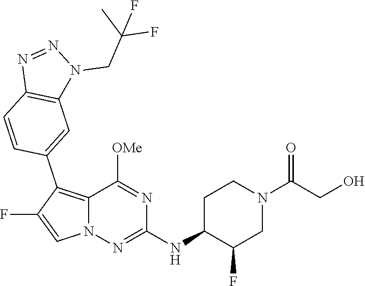 [SMILES: COc1nc(N[C@H]2CCN(C(=O)CO)C[C@H]2F)nn2cc(F)c(-c3ccc4nnn(CC(C)(F)F)c4c3)c12] |
| 1359 | 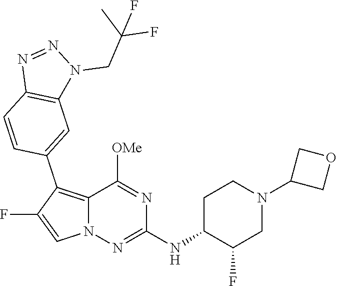 [SMILES: COc1nc(N[C@@H]2CCN(C3COC3)C[C@@H]2F)nn2cc(F)c(-c3ccc4nnn(CC(C)(F)F)c4c3)c12] |
| 1360 | 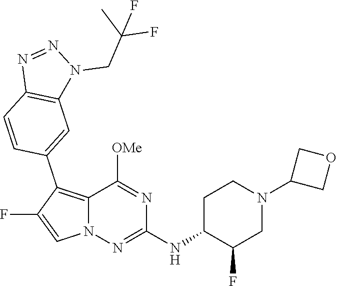 [SMILES: COc1nc(N[C@@H]2CCN(C3COC3)C[C@H]2F)nn2cc(F)c(-c3ccc4nnn(CC(C)(F)F)c4c3)c12] |
| 1361 | 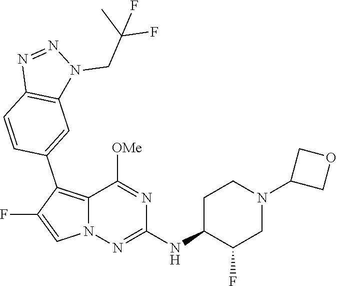 [SMILES: COc1nc(N[C@H]2CCN(C3COC3)C[C@@H]2F)nn2cc(F)c(-c3ccc4nnn(CC(C)(F)F)c4c3)c12] |
| 1362 | 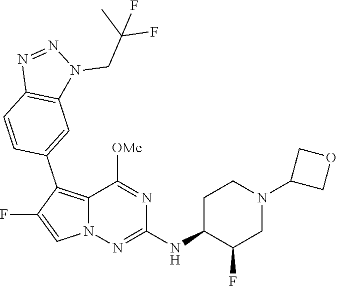 [SMILES: COc1nc(N[C@H]2CCN(C3COC3)C[C@H]2F)nn2cc(F)c(-c3ccc4nnn(CC(C)(F)F)c4c3)c12] |
| 1363 | 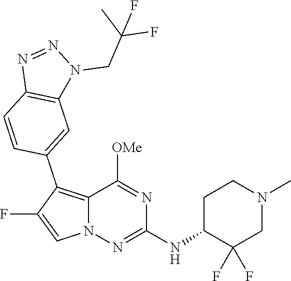 [SMILES: COc1nc(N[C@@H]2CCN(C)CC2(F)F)nn2cc(F)c(-c3ccc4nnn(CC(C)(F)F)c4c3)c12] |
| 1364 | 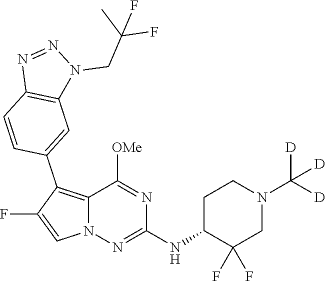 [SMILES: [2H]C([2H])([2H])N1CC[C@@H](Nc2nc(OC)c3c(-c4ccc5nnn(CC(C)(F)F)c5c4)c(F)cn3n2)C(F)(F)C1] |
| 1365 | 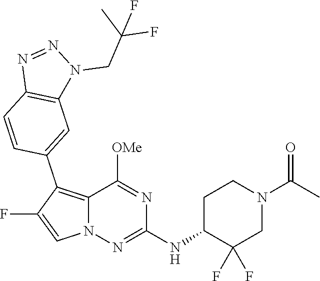 [SMILES: COc1nc(N[C@@H]2CCN(C(C)=O)CC2(F)F)nn2cc(F)c(-c3ccc4nnn(CC(C)(F)F)c4c3)c12] |
| 1366 | 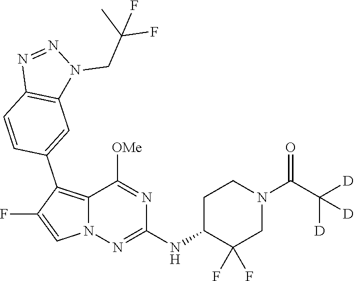 [SMILES: [2H]C([2H])([2H])C(=O)N1CC[C@@H](Nc2nc(OC)c3c(-c4ccc5nnn(CC(C)(F)F)c5c4)c(F)cn3n2)C(F)(F)C1] |
| 1367 | 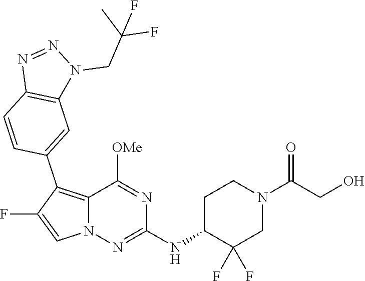 [SMILES: COc1nc(N[C@@H]2CCN(C(=O)CO)CC2(F)F)nn2cc(F)c(-c3ccc4nnn(CC(C)(F)F)c4c3)c12] |
| 1368 | 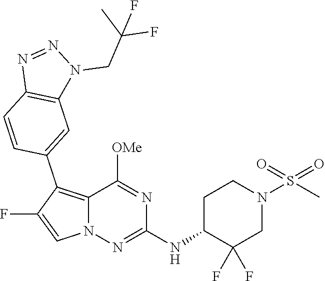 [SMILES: COc1nc(N[C@@H]2CCN(S(C)(=O)=O)CC2(F)F)nn2cc(F)c(-c3ccc4nnn(CC(C)(F)F)c4c3)c12] |
| 1369 | 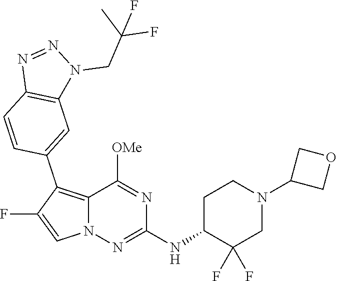 [SMILES: COc1nc(N[C@@H]2CCN(C3COC3)CC2(F)F)nn2cc(F)c(-c3ccc4nnn(CC(C)(F)F)c4c3)c12] |
| 1370 | 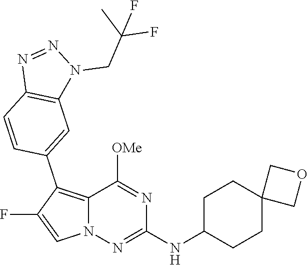 [SMILES: COc1nc(NC2CCC3(CC2)COC3)nn2cc(F)c(-c3ccc4nnn(CC(C)(F)F)c4c3)c12] |
| 1371 | 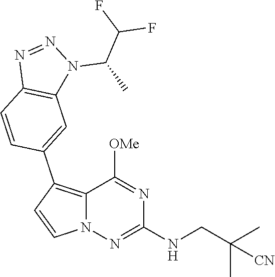 [SMILES: COc1nc(NCC(C)(C)C#N)nn2ccc(-c3ccc4nnn([C@@H](C)C(F)F)c4c3)c12] |
| 1372 | 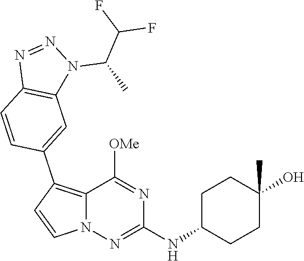 [SMILES: COc1nc(N[C@H]2CC[C@@](C)(O)CC2)nn2ccc(-c3ccc4nnn([C@@H](C)C(F)F)c4c3)c12] |
| 1373 | 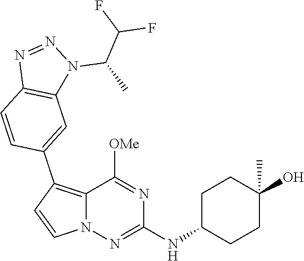 [SMILES: COc1nc(N[C@H]2CC[C@](C)(O)CC2)nn2ccc(-c3ccc4nnn([C@@H](C)C(F)F)c4c3)c12] |
| 1374 | 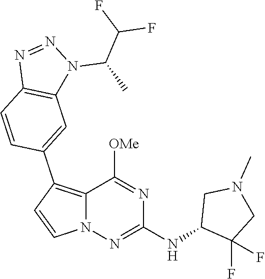 [SMILES: COc1nc(N[C@@H]2CN(C)CC2(F)F)nn2ccc(-c3ccc4nnn([C@@H](C)C(F)F)c4c3)c12] |
| 1375 | 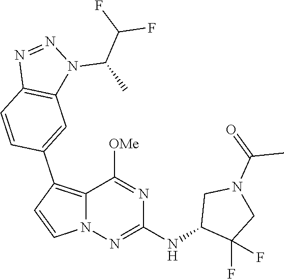 [SMILES: COc1nc(N[C@@H]2CN(C(C)=O)CC2(F)F)nn2ccc(-c3ccc4nnn([C@@H](C)C(F)F)c4c3)c12] |
| 1376 | 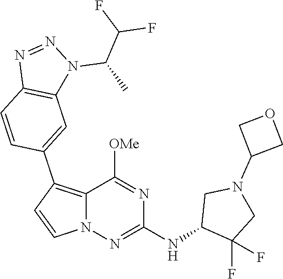 [SMILES: COc1nc(N[C@@H]2CN(C3COC3)CC2(F)F)nn2ccc(-c3ccc4nnn([C@@H](C)C(F)F)c4c3)c12] |
| 1377 | 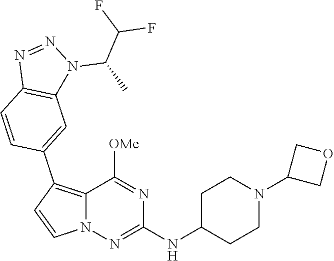 [SMILES: COc1nc(NC2CCN(C3COC3)CC2)nn2ccc(-c3ccc4nnn([C@@H](C)C(F)F)c4c3)c12] |
| 1378 | 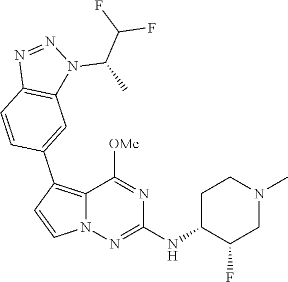 [SMILES: COc1nc(N[C@@H]2CCN(C)C[C@@H]2F)nn2ccc(-c3ccc4nnn([C@@H](C)C(F)F)c4c3)c12] |
| 1379 | 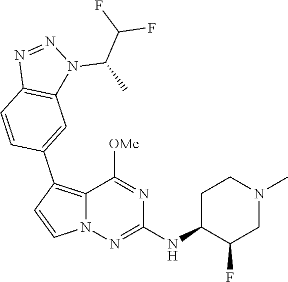 [SMILES: COc1nc(N[C@H]2CCN(C)C[C@H]2F)nn2ccc(-c3ccc4nnn([C@@H](C)C(F)F)c4c3)c12] |
| 1380 | 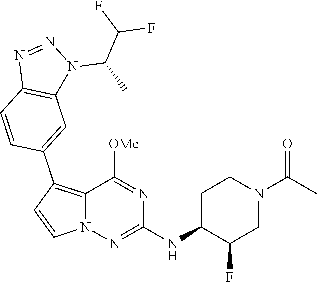 [SMILES: COc1nc(N[C@H]2CCN(C(C)=O)C[C@H]2F)nn2ccc(-c3ccc4nnn([C@@H](C)C(F)F)c4c3)c12] |
| 1381 | 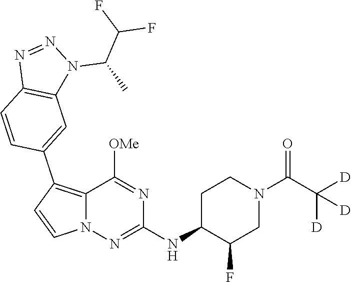 [SMILES: [2H]C([2H])([2H])C(=O)N1CC[C@H](Nc2nc(OC)c3c(-c4ccc5nnn([C@@H](C)C(F)F)c5c4)ccn3n2)[C@H](F)C1] |
| 1382 | 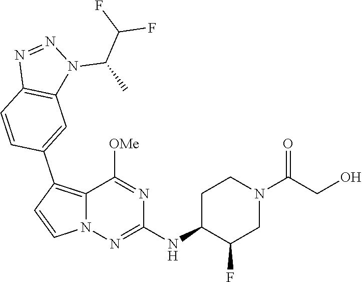 [SMILES: COc1nc(N[C@H]2CCN(C(=O)CO)C[C@H]2F)nn2ccc(-c3ccc4nnn([C@@H](C)C(F)F)c4c3)c12] |
| 1383 | 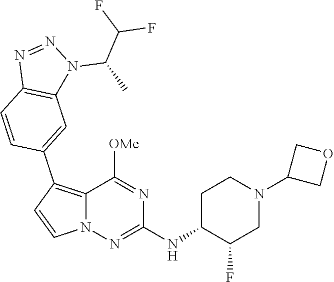 [SMILES: COc1nc(N[C@@H]2CCN(C3COC3)C[C@@H]2F)nn2ccc(-c3ccc4nnn([C@@H](C)C(F)F)c4c3)c12] |
| 1384 | 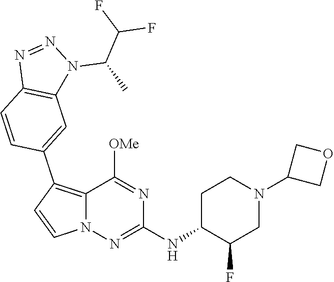 [SMILES: COc1nc(N[C@@H]2CCN(C3COC3)C[C@H]2F)nn2ccc(-c3ccc4nnn([C@@H](C)C(F)F)c4c3)c12] |
| 1385 | 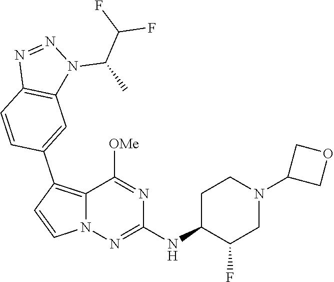 [SMILES: COc1nc(N[C@H]2CCN(C3COC3)C[C@@H]2F)nn2ccc(-c3ccc4nnn([C@@H](C)C(F)F)c4c3)c12] |
| 1386 | 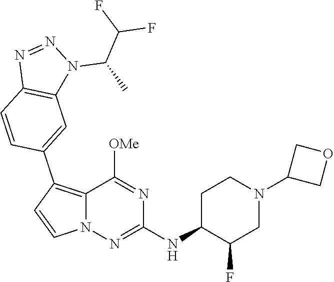 [SMILES: COc1nc(N[C@H]2CCN(C3COC3)C[C@H]2F)nn2ccc(-c3ccc4nnn([C@@H](C)C(F)F)c4c3)c12] |
| 1387 | 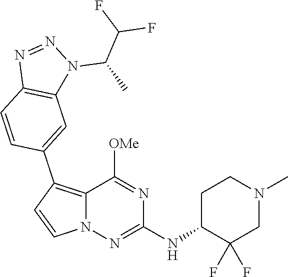 [SMILES: COc1nc(N[C@@H]2CCN(C)CC2(F)F)nn2ccc(-c3ccc4nnn([C@@H](C)C(F)F)c4c3)c12] |
| 1388 | 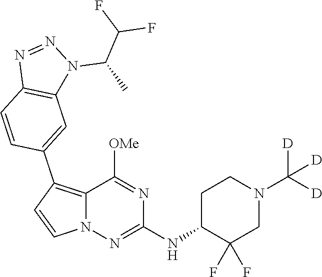 [SMILES: [2H]C([2H])([2H])N1CC[C@@H](Nc2nc(OC)c3c(-c4ccc5nnn([C@@H](C)C(F)F)c5c4)ccn3n2)C(F)(F)C1] |
| 1389 | 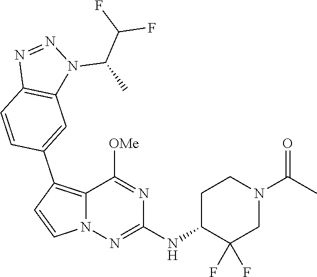 [SMILES: COc1nc(N[C@@H]2CCN(C(C)=O)CC2(F)F)nn2ccc(-c3ccc4nnn([C@@H](C)C(F)F)c4c3)c12] |
| 1390 | 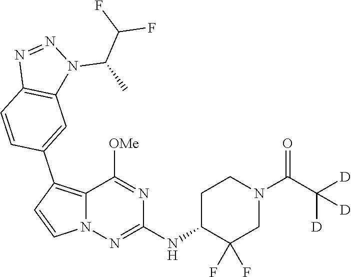 [SMILES: [2H]C([2H])([2H])C(=O)N1CC[C@@H](Nc2nc(OC)c3c(-c4ccc5nnn([C@@H](C)C(F)F)c5c4)ccn3n2)C(F)(F)C1] |
| 1391 | 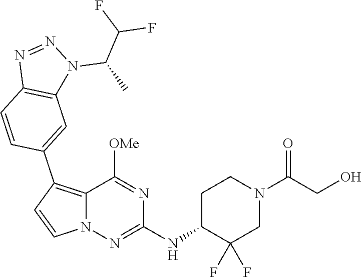 [SMILES: COc1nc(N[C@@H]2CCN(C(=O)CO)CC2(F)F)nn2ccc(-c3ccc4nnn([C@@H](C)C(F)F)c4c3)c12] |
| 1392 | 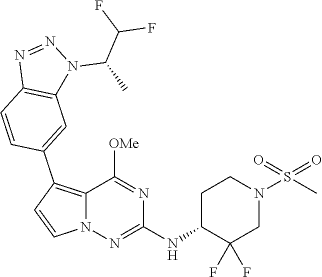 [SMILES: COc1nc(N[C@@H]2CCN(S(C)(=O)=O)CC2(F)F)nn2ccc(-c3ccc4nnn([C@@H](C)C(F)F)c4c3)c12] |
| 1393 | 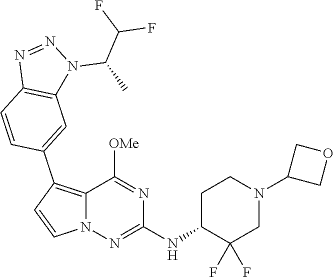 [SMILES: COc1nc(N[C@@H]2CCN(C3COC3)CC2(F)F)nn2ccc(-c3ccc4nnn([C@@H](C)C(F)F)c4c3)c12] |
| 1394 | 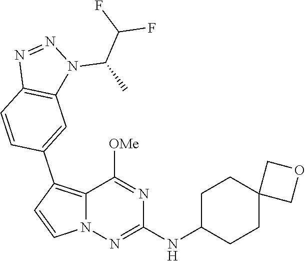 [SMILES: COc1nc(NC2CCC3(CC2)COC3)nn2ccc(-c3ccc4nnn([C@@H](C)C(F)F)c4c3)c12] |
| 1395 | 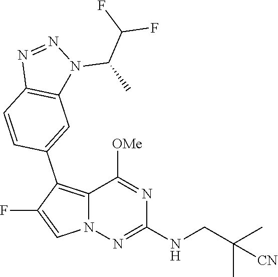 [SMILES: COc1nc(NCC(C)(C)C#N)nn2cc(F)c(-c3ccc4nnn([C@@H](C)C(F)F)c4c3)c12] |
| 1396 | 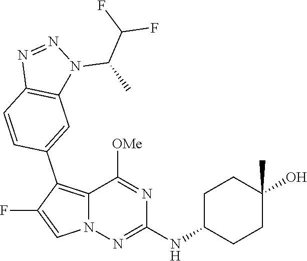 [SMILES: COc1nc(N[C@H]2CC[C@@](C)(O)CC2)nn2cc(F)c(-c3ccc4nnn([C@@H](C)C(F)F)c4c3)c12] |
| 1397 | 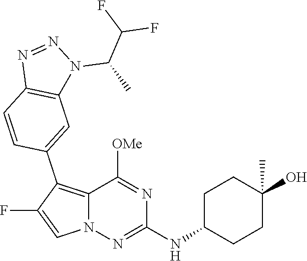 [SMILES: COc1nc(N[C@H]2CC[C@](C)(O)CC2)nn2cc(F)c(-c3ccc4nnn([C@@H](C)C(F)F)c4c3)c12] |
| 1398 | 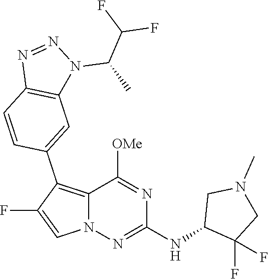 [SMILES: COc1nc(N[C@@H]2CN(C)CC2(F)F)nn2cc(F)c(-c3ccc4nnn([C@@H](C)C(F)F)c4c3)c12] |
| 1399 | 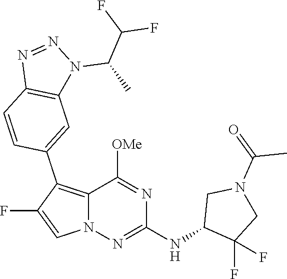 [SMILES: COc1nc(N[C@@H]2CN(C(C)=O)CC2(F)F)nn2cc(F)c(-c3ccc4nnn([C@@H](C)C(F)F)c4c3)c12] |
| 1400 | 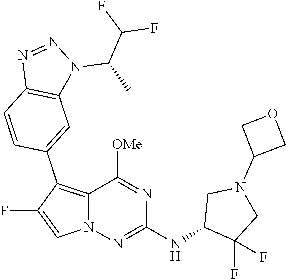 [SMILES: COc1nc(N[C@@H]2CN(C3COC3)CC2(F)F)nn2cc(F)c(-c3ccc4nnn([C@@H](C)C(F)F)c4c3)c12] |
| 1401 | 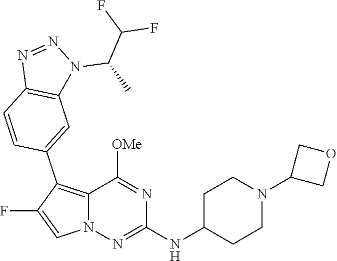 [SMILES: COc1nc(NC2CCN(C3COC3)CC2)nn2cc(F)c(-c3ccc4nnn([C@@H](C)C(F)F)c4c3)c12] |
| 1402 | 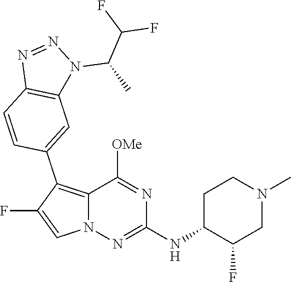 [SMILES: COc1nc(N[C@@H]2CCN(C)C[C@@H]2F)nn2cc(F)c(-c3ccc4nnn([C@@H](C)C(F)F)c4c3)c12] |
| 1403 | 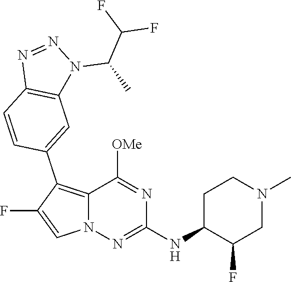 [SMILES: COc1nc(N[C@H]2CCN(C)C[C@H]2F)nn2cc(F)c(-c3ccc4nnn([C@@H](C)C(F)F)c4c3)c12] |
| 1404 | 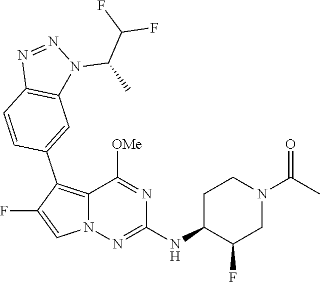 [SMILES: COc1nc(N[C@H]2CCN(C(C)=O)C[C@H]2F)nn2cc(F)c(-c3ccc4nnn([C@@H](C)C(F)F)c4c3)c12] |
| 1405 | 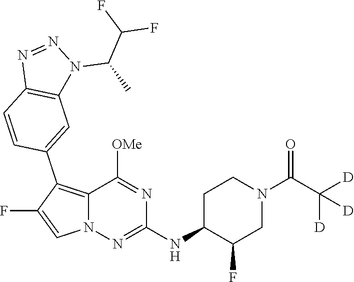 [SMILES: [2H]C([2H])([2H])C(=O)N1CC[C@H](Nc2nc(OC)c3c(-c4ccc5nnn([C@@H](C)C(F)F)c5c4)c(F)cn3n2)[C@H](F)C1] |
| 1406 | 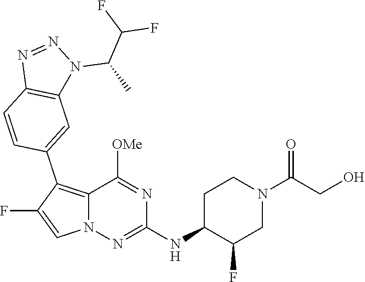 [SMILES: COc1nc(N[C@H]2CCN(C(=O)CO)C[C@H]2F)nn2cc(F)c(-c3ccc4nnn([C@@H](C)C(F)F)c4c3)c12] |
| 1407 | 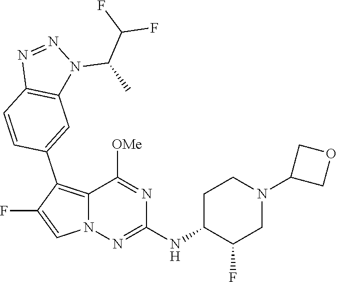 [SMILES: COc1nc(N[C@@H]2CCN(C3COC3)C[C@@H]2F)nn2cc(F)c(-c3ccc4nnn([C@@H](C)C(F)F)c4c3)c12] |
| 1408 | 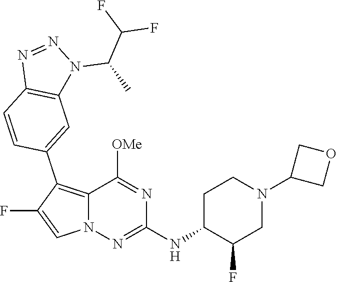 [SMILES: COc1nc(N[C@@H]2CCN(C3COC3)C[C@H]2F)nn2cc(F)c(-c3ccc4nnn([C@@H](C)C(F)F)c4c3)c12] |
| 1409 | 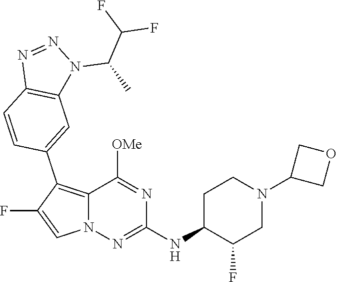 [SMILES: COc1nc(N[C@H]2CCN(C3COC3)C[C@@H]2F)nn2cc(F)c(-c3ccc4nnn([C@@H](C)C(F)F)c4c3)c12] |
| 1410 | 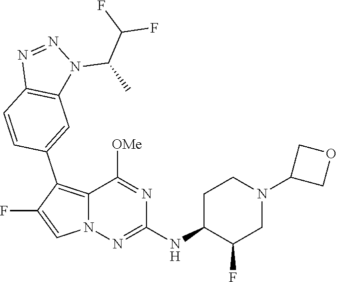 [SMILES: COc1nc(N[C@H]2CCN(C3COC3)C[C@H]2F)nn2cc(F)c(-c3ccc4nnn([C@@H](C)C(F)F)c4c3)c12] |
| 1411 | 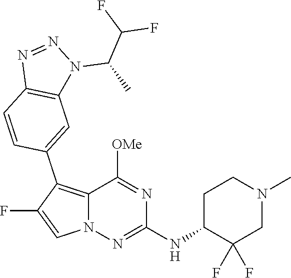 [SMILES: COc1nc(N[C@@H]2CCN(C)CC2(F)F)nn2cc(F)c(-c3ccc4nnn([C@@H](C)C(F)F)c4c3)c12] |
| 1412 | 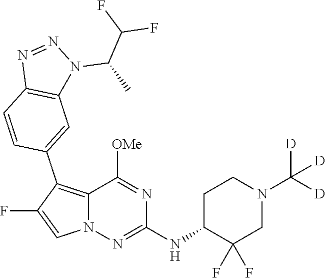 [SMILES: [2H]C([2H])([2H])N1CC[C@@H](Nc2nc(OC)c3c(-c4ccc5nnn([C@@H](C)C(F)F)c5c4)c(F)cn3n2)C(F)(F)C1] |
| 1413 | 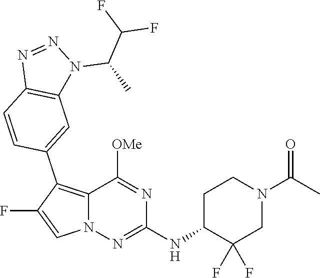 [SMILES: COc1nc(N[C@@H]2CCN(C(C)=O)CC2(F)F)nn2cc(F)c(-c3ccc4nnn([C@@H](C)C(F)F)c4c3)c12] |
| 1414 | 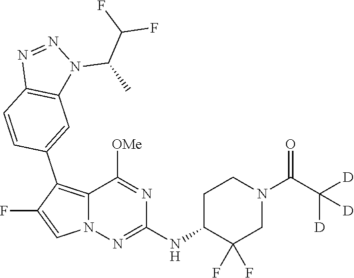 [SMILES: [2H]C([2H])([2H])C(=O)N1CC[C@@H](Nc2nc(OC)c3c(-c4ccc5nnn([C@@H](C)C(F)F)c5c4)c(F)cn3n2)C(F)(F)C1] |
| 1415 | 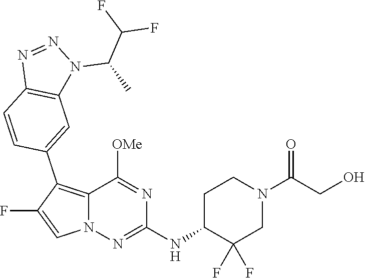 [SMILES: COc1nc(N[C@@H]2CCN(C(=O)CO)CC2(F)F)nn2cc(F)c(-c3ccc4nnn([C@@H](C)C(F)F)c4c3)c12] |
| 1416 | 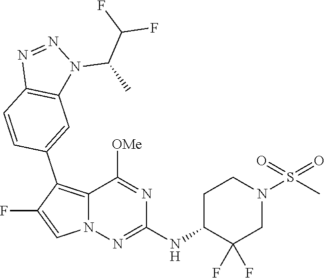 [SMILES: COc1nc(N[C@@H]2CCN(S(C)(=O)=O)CC2(F)F)nn2cc(F)c(-c3ccc4nnn([C@@H](C)C(F)F)c4c3)c12] |
| 1417 | 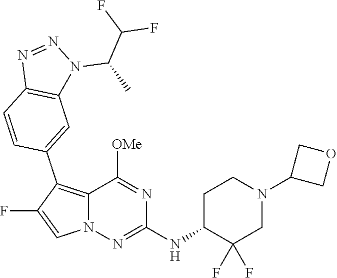 [SMILES: COc1nc(N[C@@H]2CCN(C3COC3)CC2(F)F)nn2cc(F)c(-c3ccc4nnn([C@@H](C)C(F)F)c4c3)c12] |
| 1418 | 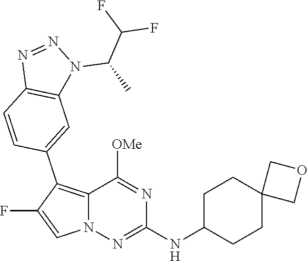 [SMILES: COc1nc(NC2CCC3(CC2)COC3)nn2cc(F)c(-c3ccc4nnn([C@@H](C)C(F)F)c4c3)c12] |
| 1419 | 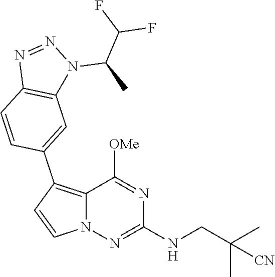 [SMILES: COc1nc(NCC(C)(C)C#N)nn2ccc(-c3ccc4nnn([C@H](C)C(F)F)c4c3)c12] |
| 1420 | 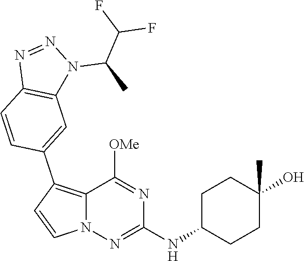 [SMILES: COc1nc(N[C@H]2CC[C@@](C)(O)CC2)nn2ccc(-c3ccc4nnn([C@H](C)C(F)F)c4c3)c12] |
| 1421 | 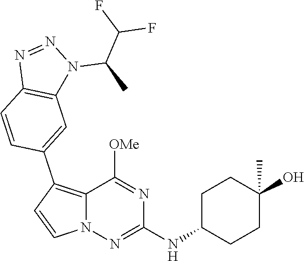 [SMILES: COc1nc(N[C@H]2CC[C@](C)(O)CC2)nn2ccc(-c3ccc4nnn([C@H](C)C(F)F)c4c3)c12] |
| 1422 | 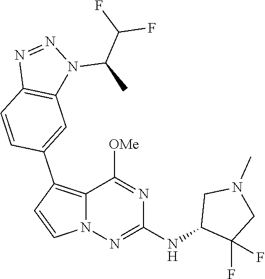 [SMILES: COc1nc(N[C@@H]2CN(C)CC2(F)F)nn2ccc(-c3ccc4nnn([C@H](C)C(F)F)c4c3)c12] |
| 1423 | 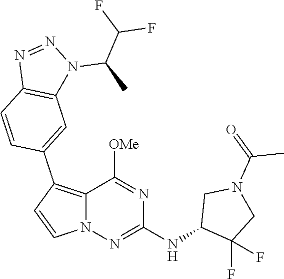 [SMILES: COc1nc(N[C@@H]2CN(C(C)=O)CC2(F)F)nn2ccc(-c3ccc4nnn([C@H](C)C(F)F)c4c3)c12] |
| 1424 | 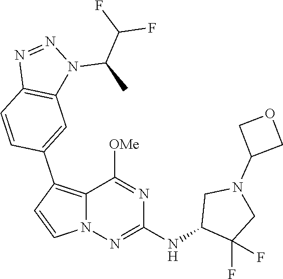 [SMILES: COc1nc(N[C@@H]2CN(C3COC3)CC2(F)F)nn2ccc(-c3ccc4nnn([C@H](C)C(F)F)c4c3)c12] |
| 1425 | 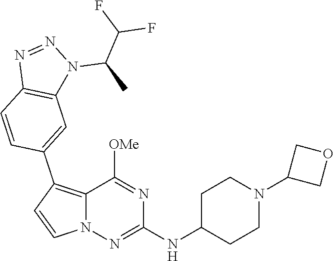 [SMILES: COc1nc(NC2CCN(C3COC3)CC2)nn2ccc(-c3ccc4nnn([C@H](C)C(F)F)c4c3)c12] |
| 1426 | 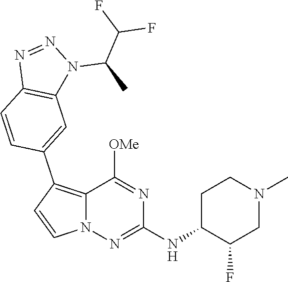 [SMILES: COc1nc(N[C@@H]2CCN(C)C[C@@H]2F)nn2ccc(-c3ccc4nnn([C@H](C)C(F)F)c4c3)c12] |
| 1427 | 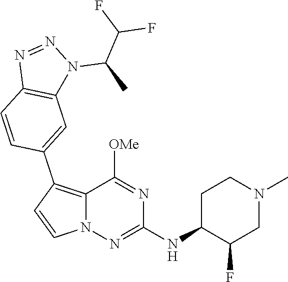 [SMILES: COc1nc(N[C@H]2CCN(C)C[C@H]2F)nn2ccc(-c3ccc4nnn([C@H](C)C(F)F)c4c3)c12] |
| 1428 | 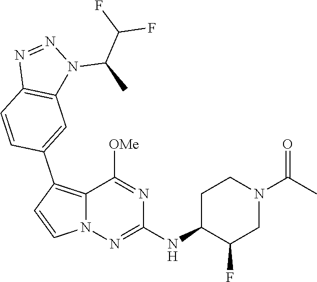 [SMILES: COc1nc(N[C@H]2CCN(C(C)=O)C[C@H]2F)nn2ccc(-c3ccc4nnn([C@H](C)C(F)F)c4c3)c12] |
| 1429 | 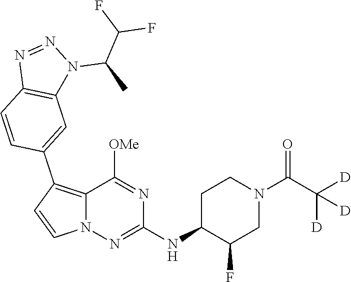 [SMILES: [2H]C([2H])([2H])C(=O)N1CC[C@H](Nc2nc(OC)c3c(-c4ccc5nnn([C@H](C)C(F)F)c5c4)ccn3n2)[C@H](F)C1] |
| 1430 | 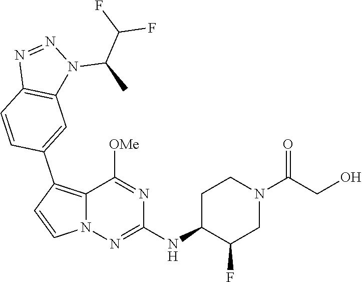 [SMILES: COc1nc(N[C@H]2CCN(C(=O)CO)C[C@H]2F)nn2ccc(-c3ccc4nnn([C@H](C)C(F)F)c4c3)c12] |
| 1431 | 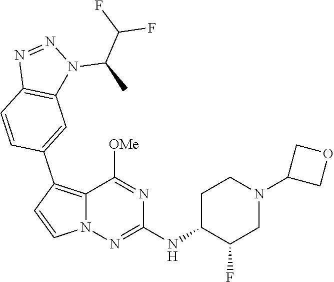 [SMILES: COc1nc(N[C@@H]2CCN(C3COC3)C[C@@H]2F)nn2ccc(-c3ccc4nnn([C@H](C)C(F)F)c4c3)c12] |
| 1432 | 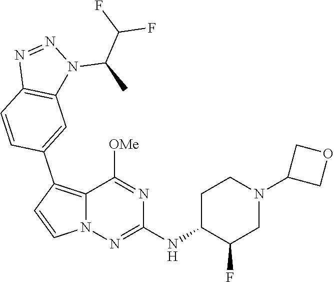 [SMILES: COc1nc(N[C@@H]2CCN(C3COC3)C[C@H]2F)nn2ccc(-c3ccc4nnn([C@H](C)C(F)F)c4c3)c12] |
| 1433 | 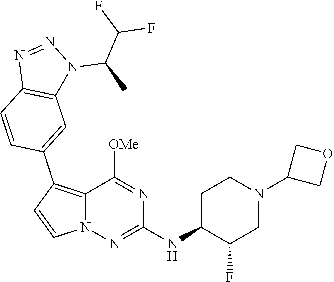 [SMILES: COc1nc(N[C@H]2CCN(C3COC3)C[C@@H]2F)nn2ccc(-c3ccc4nnn([C@H](C)C(F)F)c4c3)c12] |
| 1434 | 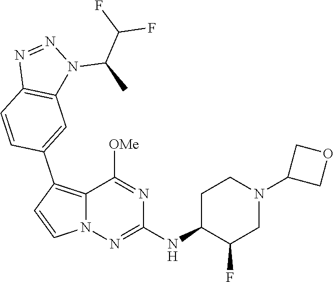 [SMILES: COc1nc(N[C@H]2CCN(C3COC3)C[C@H]2F)nn2ccc(-c3ccc4nnn([C@H](C)C(F)F)c4c3)c12] |
| 1435 | 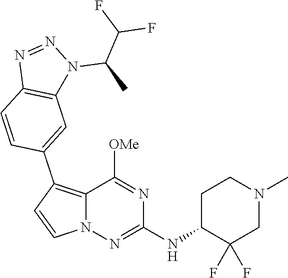 [SMILES: COc1nc(N[C@@H]2CCN(C)CC2(F)F)nn2ccc(-c3ccc4nnn([C@H](C)C(F)F)c4c3)c12] |
| 1436 | 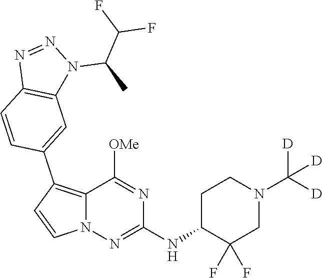 [SMILES: [2H]C([2H])([2H])N1CC[C@@H](Nc2nc(OC)c3c(-c4ccc5nnn([C@H](C)C(F)F)c5c4)ccn3n2)C(F)(F)C1] |
| 1437 | 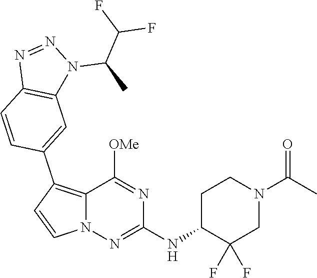 [SMILES: COc1nc(N[C@@H]2CCN(C(C)=O)CC2(F)F)nn2ccc(-c3ccc4nnn([C@H](C)C(F)F)c4c3)c12] |
| 1438 | 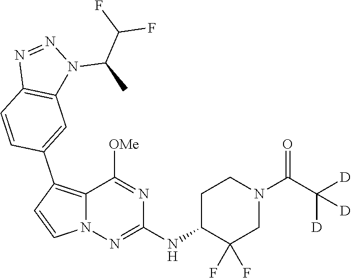 [SMILES: [2H]C([2H])([2H])C(=O)N1CC[C@@H](Nc2nc(OC)c3c(-c4ccc5nnn([C@H](C)C(F)F)c5c4)ccn3n2)C(F)(F)C1] |
| 1439 | 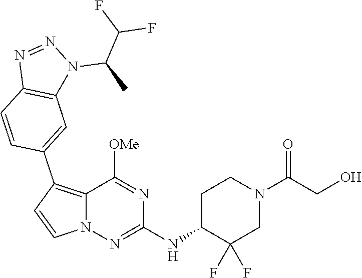 [SMILES: COc1nc(N[C@@H]2CCN(C(=O)CO)CC2(F)F)nn2ccc(-c3ccc4nnn([C@H](C)C(F)F)c4c3)c12] |
| 1440 | 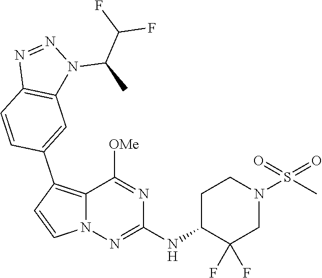 [SMILES: COc1nc(N[C@@H]2CCN(S(C)(=O)=O)CC2(F)F)nn2ccc(-c3ccc4nnn([C@H](C)C(F)F)c4c3)c12] |
| 1441 | 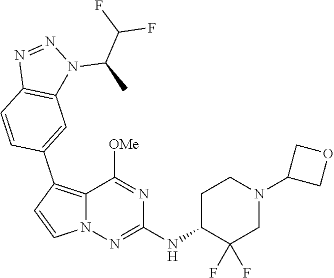 [SMILES: COc1nc(N[C@@H]2CCN(C3COC3)CC2(F)F)nn2ccc(-c3ccc4nnn([C@H](C)C(F)F)c4c3)c12] |
| 1442 | 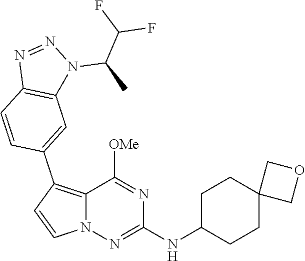 [SMILES: COc1nc(NC2CCC3(CC2)COC3)nn2ccc(-c3ccc4nnn([C@H](C)C(F)F)c4c3)c12] |
| 1443 | 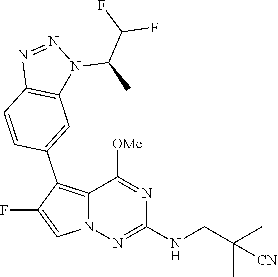 [SMILES: COc1nc(NCC(C)(C)C#N)nn2cc(F)c(-c3ccc4nnn([C@H](C)C(F)F)c4c3)c12] |
| 1444 | 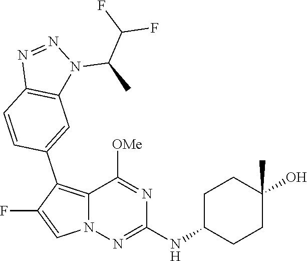 [SMILES: COc1nc(N[C@H]2CC[C@@](C)(O)CC2)nn2cc(F)c(-c3ccc4nnn([C@H](C)C(F)F)c4c3)c12] |
| 1445 | 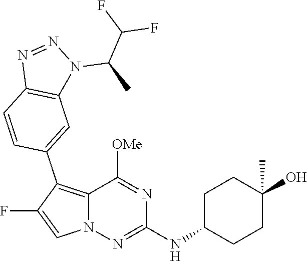 [SMILES: COc1nc(N[C@H]2CC[C@](C)(O)CC2)nn2cc(F)c(-c3ccc4nnn([C@H](C)C(F)F)c4c3)c12] |
| 1446 | 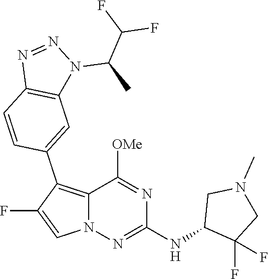 [SMILES: COc1nc(N[C@@H]2CN(C)CC2(F)F)nn2cc(F)c(-c3ccc4nnn([C@H](C)C(F)F)c4c3)c12] |
| 1447 | 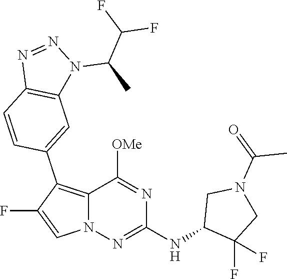 [SMILES: COc1nc(N[C@@H]2CN(C(C)=O)CC2(F)F)nn2cc(F)c(-c3ccc4nnn([C@H](C)C(F)F)c4c3)c12] |
| 1448 | 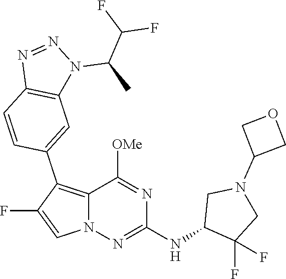 [SMILES: COc1nc(N[C@@H]2CN(C3COC3)CC2(F)F)nn2cc(F)c(-c3ccc4nnn([C@H](C)C(F)F)c4c3)c12] |
| 1449 | 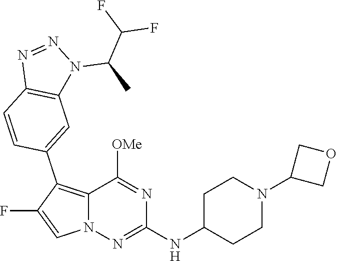 [SMILES: COc1nc(NC2CCN(C3COC3)CC2)nn2cc(F)c(-c3ccc4nnn([C@H](C)C(F)F)c4c3)c12] |
| 1450 | 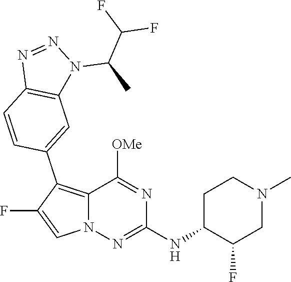 [SMILES: COc1nc(N[C@@H]2CCN(C)C[C@@H]2F)nn2cc(F)c(-c3ccc4nnn([C@H](C)C(F)F)c4c3)c12] |
| 1451 | 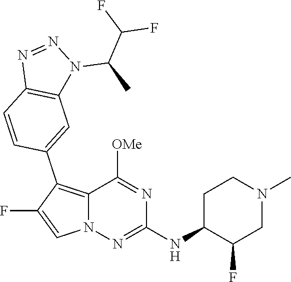 [SMILES: COc1nc(N[C@H]2CCN(C)C[C@H]2F)nn2cc(F)c(-c3ccc4nnn([C@H](C)C(F)F)c4c3)c12] |
| 1452 | 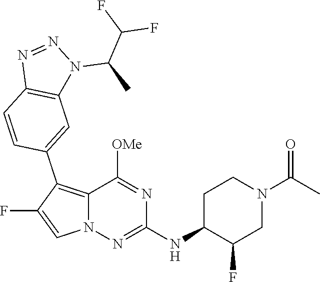 [SMILES: COc1nc(N[C@H]2CCN(C(C)=O)C[C@H]2F)nn2cc(F)c(-c3ccc4nnn([C@H](C)C(F)F)c4c3)c12] |
| 1453 | 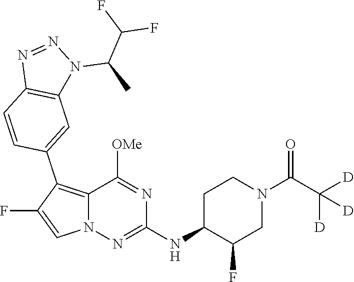 [SMILES: [2H]C([2H])([2H])C(=O)N1CC[C@H](Nc2nc(OC)c3c(-c4ccc5nnn([C@H](C)C(F)F)c5c4)c(F)cn3n2)[C@H](F)C1] |
| 1454 | 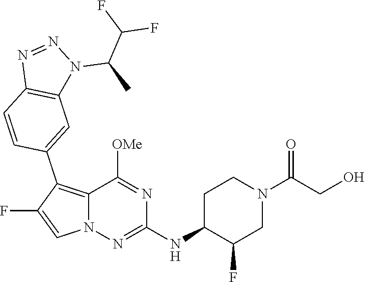 [SMILES: COc1nc(N[C@H]2CCN(C(=O)CO)C[C@H]2F)nn2cc(F)c(-c3ccc4nnn([C@H](C)C(F)F)c4c3)c12] |
| 1455 | 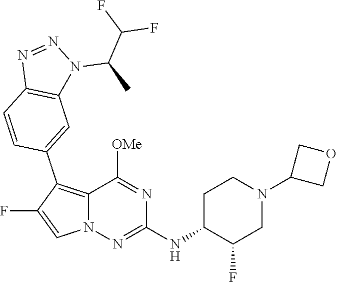 [SMILES: COc1nc(N[C@@H]2CCN(C3COC3)C[C@@H]2F)nn2cc(F)c(-c3ccc4nnn([C@H](C)C(F)F)c4c3)c12] |
| 1456 | 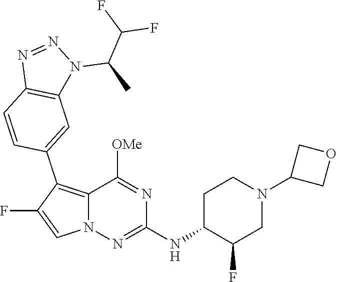 [SMILES: COc1nc(N[C@@H]2CCN(C3COC3)C[C@H]2F)nn2cc(F)c(-c3ccc4nnn([C@H](C)C(F)F)c4c3)c12] |
| 1457 | 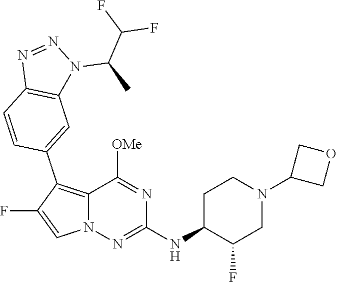 [SMILES: COc1nc(N[C@H]2CCN(C3COC3)C[C@@H]2F)nn2cc(F)c(-c3ccc4nnn([C@H](C)C(F)F)c4c3)c12] |
| 1458 | 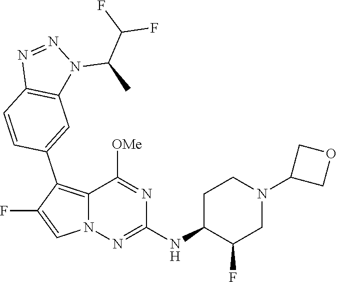 [SMILES: COc1nc(N[C@H]2CCN(C3COC3)C[C@H]2F)nn2cc(F)c(-c3ccc4nnn([C@H](C)C(F)F)c4c3)c12] |
| 1459 | 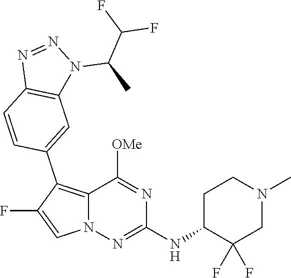 [SMILES: COc1nc(N[C@@H]2CCN(C)CC2(F)F)nn2cc(F)c(-c3ccc4nnn([C@H](C)C(F)F)c4c3)c12] |
| 1460 | 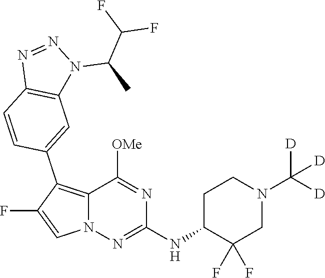 [SMILES: [2H]C([2H])([2H])N1CC[C@@H](Nc2nc(OC)c3c(-c4ccc5nnn([C@H](C)C(F)F)c5c4)c(F)cn3n2)C(F)(F)C1] |
| 1461 | 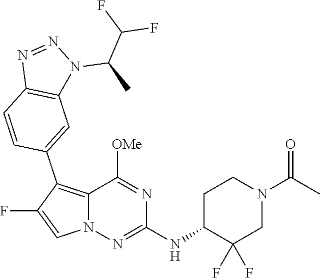 [SMILES: COc1nc(N[C@@H]2CCN(C(C)=O)CC2(F)F)nn2cc(F)c(-c3ccc4nnn([C@H](C)C(F)F)c4c3)c12] |
| 1462 | 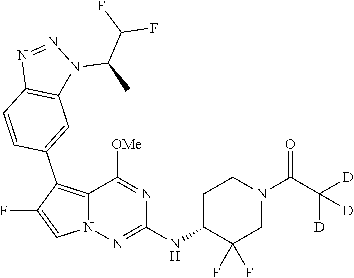 [SMILES: [2H]C([2H])([2H])C(=O)N1CC[C@@H](Nc2nc(OC)c3c(-c4ccc5nnn([C@H](C)C(F)F)c5c4)c(F)cn3n2)C(F)(F)C1] |
| 1463 | 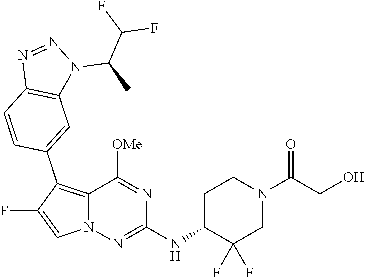 [SMILES: COc1nc(N[C@@H]2CCN(C(=O)CO)CC2(F)F)nn2cc(F)c(-c3ccc4nnn([C@H](C)C(F)F)c4c3)c12] |
| 1464 | 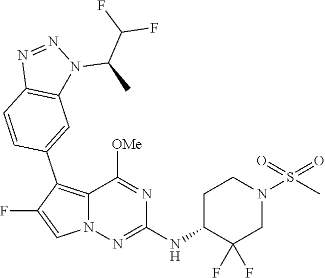 [SMILES: COc1nc(N[C@@H]2CCN(S(C)(=O)=O)CC2(F)F)nn2cc(F)c(-c3ccc4nnn([C@H](C)C(F)F)c4c3)c12] |
| 1465 | 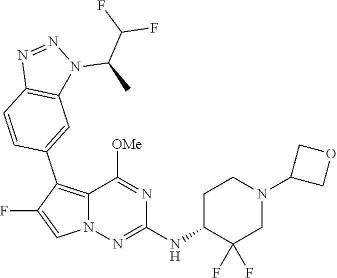 [SMILES: COc1nc(N[C@@H]2CCN(C3COC3)CC2(F)F)nn2cc(F)c(-c3ccc4nnn([C@H](C)C(F)F)c4c3)c12] |
| 1466 | 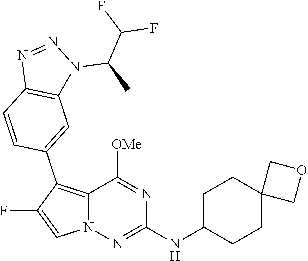 [SMILES: COc1nc(NC2CCC3(CC2)COC3)nn2cc(F)c(-c3ccc4nnn([C@H](C)C(F)F)c4c3)c12] |
| 1467 | 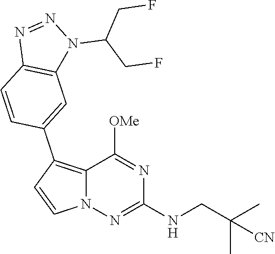 [SMILES: COc1nc(NCC(C)(C)C#N)nn2ccc(-c3ccc4nnn(C(CF)CF)c4c3)c12] |
| 1468 | 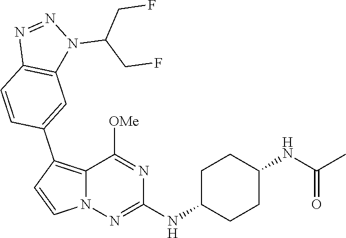 [SMILES: COc1nc(N[C@H]2CC[C@@H](NC(C)=O)CC2)nn2ccc(-c3ccc4nnn(C(CF)CF)c4c3)c12] |
| 1469 | 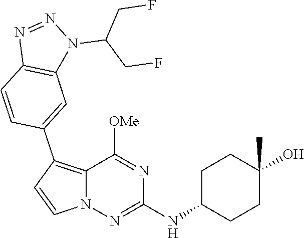 [SMILES: COc1nc(N[C@H]2CC[C@@](C)(O)CC2)nn2ccc(-c3ccc4nnn(C(CF)CF)c4c3)c12] |
| 1470 | 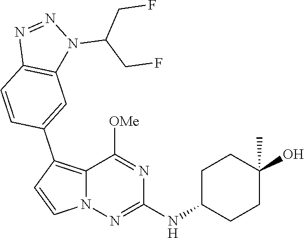 [SMILES: COc1nc(N[C@H]2CC[C@](C)(O)CC2)nn2ccc(-c3ccc4nnn(C(CF)CF)c4c3)c12] |
| 1471 | 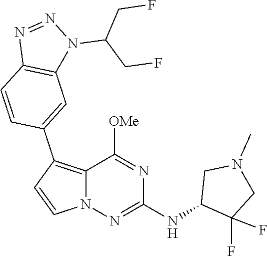 [SMILES: COc1nc(N[C@@H]2CN(C)CC2(F)F)nn2ccc(-c3ccc4nnn(C(CF)CF)c4c3)c12] |
| 1472 | 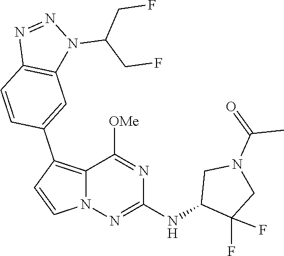 [SMILES: COc1nc(N[C@@H]2CN(C(C)=O)CC2(F)F)nn2ccc(-c3ccc4nnn(C(CF)CF)c4c3)c12] |
| 1473 | 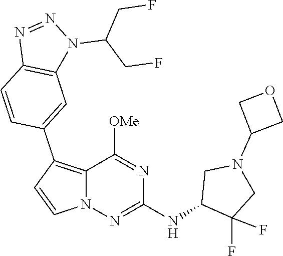 [SMILES: COc1nc(N[C@@H]2CN(C3COC3)CC2(F)F)nn2ccc(-c3ccc4nnn(C(CF)CF)c4c3)c12] |
| 1474 | 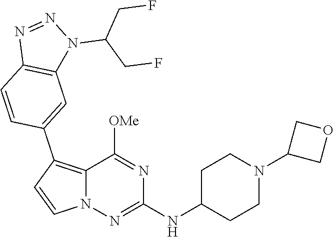 [SMILES: COc1nc(NC2CCN(C3COC3)CC2)nn2ccc(-c3ccc4nnn(C(CF)CF)c4c3)c12] |
| 1475 | 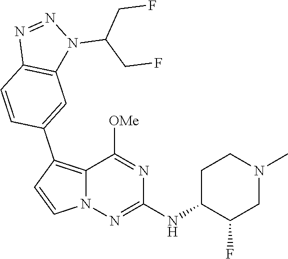 [SMILES: COc1nc(N[C@@H]2CCN(C)C[C@@H]2F)nn2ccc(-c3ccc4nnn(C(CF)CF)c4c3)c12] |
| 1476 | 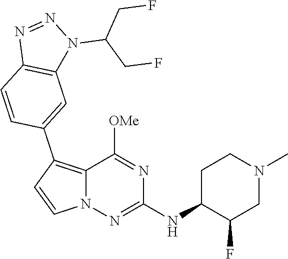 [SMILES: COc1nc(N[C@H]2CCN(C)C[C@H]2F)nn2ccc(-c3ccc4nnn(C(CF)CF)c4c3)c12] |
| 1477 | 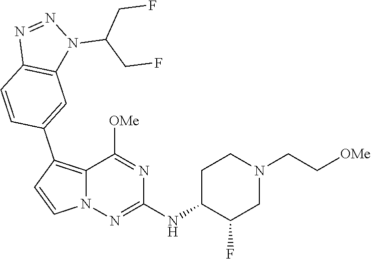 [SMILES: COCCN1CC[C@@H](Nc2nc(OC)c3c(-c4ccc5nnn(C(CF)CF)c5c4)ccn3n2)[C@@H](F)C1] |
| 1478 | 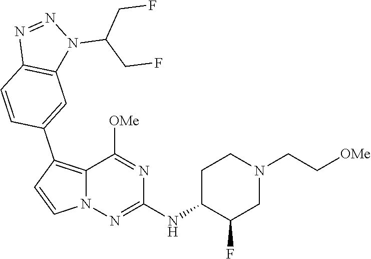 [SMILES: COCCN1CC[C@@H](Nc2nc(OC)c3c(-c4ccc5nnn(C(CF)CF)c5c4)ccn3n2)[C@H](F)C1] |
| 1479 | 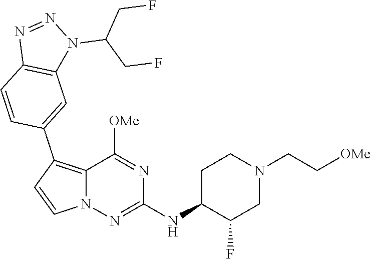 [SMILES: COCCN1CC[C@H](Nc2nc(OC)c3c(-c4ccc5nnn(C(CF)CF)c5c4)ccn3n2)[C@@H](F)C1] |
| 1480 | 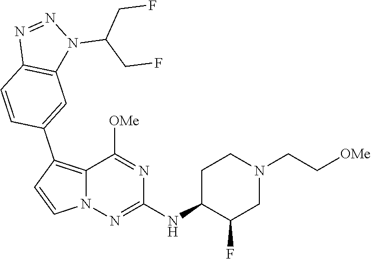 [SMILES: COCCN1CC[C@H](Nc2nc(OC)c3c(-c4ccc5nnn(C(CF)CF)c5c4)ccn3n2)[C@H](F)C1] |
| 1481 | 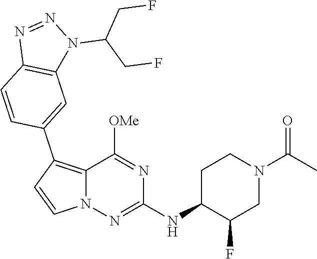 [SMILES: COc1nc(N[C@H]2CCN(C(C)=O)C[C@H]2F)nn2ccc(-c3ccc4nnn(C(CF)CF)c4c3)c12] |
| 1482 | 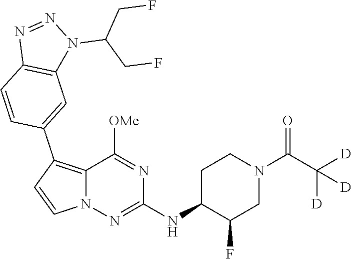 [SMILES: [2H]C([2H])([2H])C(=O)N1CC[C@H](Nc2nc(OC)c3c(-c4ccc5nnn(C(CF)CF)c5c4)ccn3n2)[C@H](F)C1] |
| 1483 | 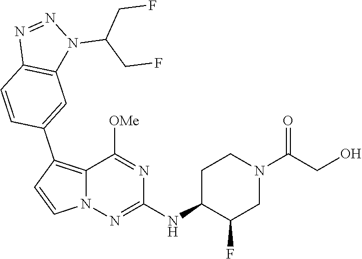 [SMILES: COc1nc(N[C@H]2CCN(C(=O)CO)C[C@H]2F)nn2ccc(-c3ccc4nnn(C(CF)CF)c4c3)c12] |
| 1484 | 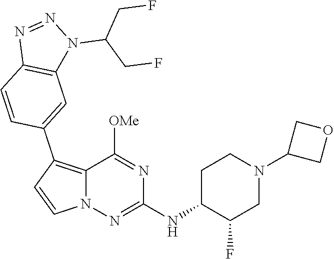 [SMILES: COc1nc(N[C@@H]2CCN(C3COC3)C[C@@H]2F)nn2ccc(-c3ccc4nnn(C(CF)CF)c4c3)c12] |
| 1485 | 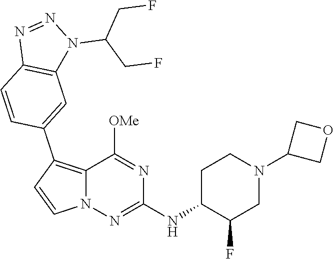 [SMILES: COc1nc(N[C@@H]2CCN(C3COC3)C[C@H]2F)nn2ccc(-c3ccc4nnn(C(CF)CF)c4c3)c12] |
| 1486 | 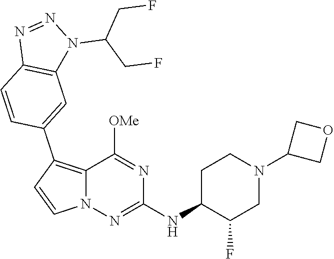 [SMILES: COc1nc(N[C@H]2CCN(C3COC3)C[C@@H]2F)nn2ccc(-c3ccc4nnn(C(CF)CF)c4c3)c12] |
| 1487 | 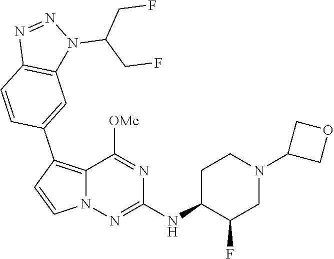 [SMILES: COc1nc(N[C@H]2CCN(C3COC3)C[C@H]2F)nn2ccc(-c3ccc4nnn(C(CF)CF)c4c3)c12] |
| 1488 | 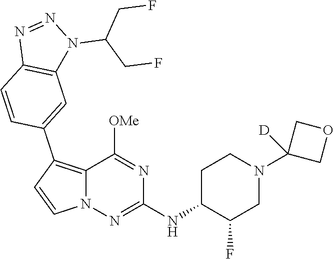 [SMILES: [2H]C1(N2CC[C@@H](Nc3nc(OC)c4c(-c5ccc6nnn(C(CF)CF)c6c5)ccn4n3)[C@@H](F)C2)COC1] |
| 1489 | 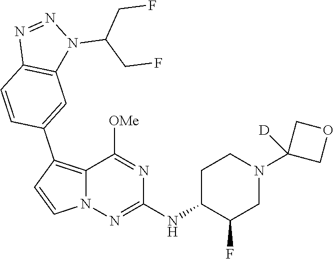 [SMILES: [2H]C1(N2CC[C@@H](Nc3nc(OC)c4c(-c5ccc6nnn(C(CF)CF)c6c5)ccn4n3)[C@H](F)C2)COC1] |
| 1490 | 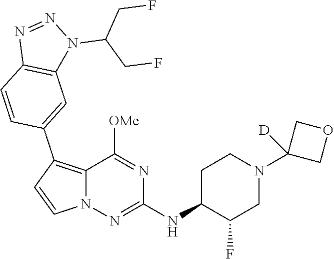 [SMILES: [2H]C1(N2CC[C@H](Nc3nc(OC)c4c(-c5ccc6nnn(C(CF)CF)c6c5)ccn4n3)[C@@H](F)C2)COC1] |
| 1491 | 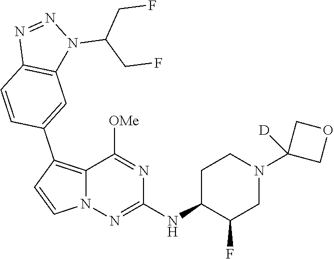 [SMILES: [2H]C1(N2CC[C@H](Nc3nc(OC)c4c(-c5ccc6nnn(C(CF)CF)c6c5)ccn4n3)[C@H](F)C2)COC1] |
| 1492 | 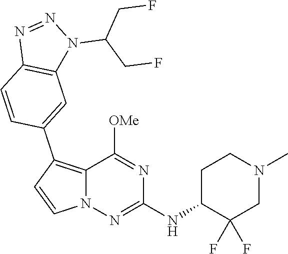 [SMILES: COc1nc(N[C@@H]2CCN(C)CC2(F)F)nn2ccc(-c3ccc4nnn(C(CF)CF)c4c3)c12] |
| 1493 | 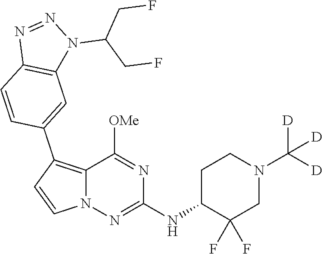 [SMILES: [2H]C([2H])([2H])N1CC[C@@H](Nc2nc(OC)c3c(-c4ccc5nnn(C(CF)CF)c5c4)ccn3n2)C(F)(F)C1] |
| 1494 | 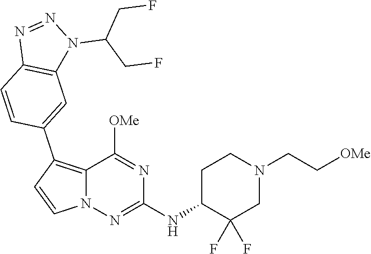 [SMILES: COCCN1CC[C@@H](Nc2nc(OC)c3c(-c4ccc5nnn(C(CF)CF)c5c4)ccn3n2)C(F)(F)C1] |
| 1495 | 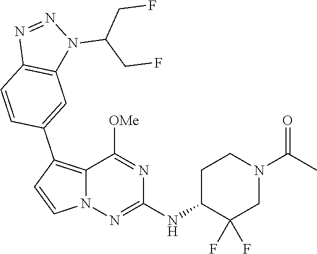 [SMILES: COc1nc(N[C@@H]2CCN(C(C)=O)CC2(F)F)nn2ccc(-c3ccc4nnn(C(CF)CF)c4c3)c12] |
| 1496 | 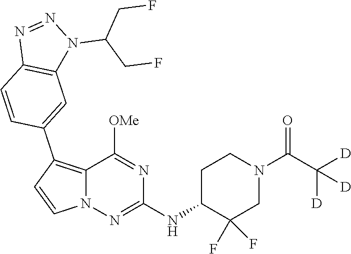 [SMILES: [2H]C([2H])([2H])C(=O)N1CC[C@@H](Nc2nc(OC)c3c(-c4ccc5nnn(C(CF)CF)c5c4)ccn3n2)C(F)(F)C1] |
| 1497 | 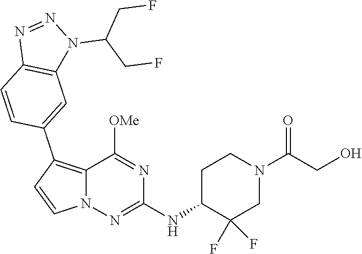 [SMILES: COc1nc(N[C@@H]2CCN(C(=O)CO)CC2(F)F)nn2ccc(-c3ccc4nnn(C(CF)CF)c4c3)c12] |
| 1498 | 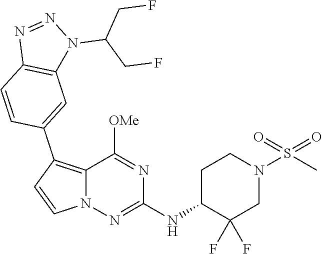 [SMILES: COc1nc(N[C@@H]2CCN(S(C)(=O)=O)CC2(F)F)nn2ccc(-c3ccc4nnn(C(CF)CF)c4c3)c12] |
| 1499 | 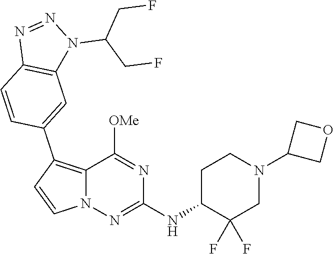 [SMILES: COc1nc(N[C@@H]2CCN(C3COC3)CC2(F)F)nn2ccc(-c3ccc4nnn(C(CF)CF)c4c3)c12] |
| 1500 | 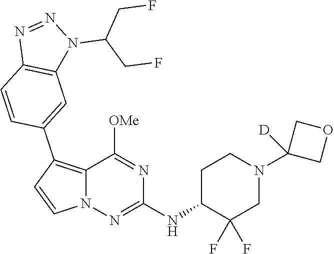 [SMILES: [2H]C1(N2CC[C@@H](Nc3nc(OC)c4c(-c5ccc6nnn(C(CF)CF)c6c5)ccn4n3)C(F)(F)C2)COC1] |
| 1501 | 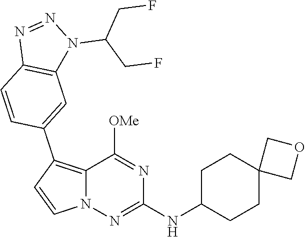 [SMILES: COc1nc(NC2CCC3(CC2)COC3)nn2ccc(-c3ccc4nnn(C(CF)CF)c4c3)c12] |
| 1502 | 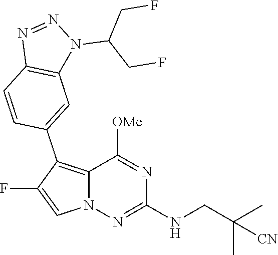 [SMILES: COc1nc(NCC(C)(C)C#N)nn2cc(F)c(-c3ccc4nnn(C(CF)CF)c4c3)c12] |
| 1503 | 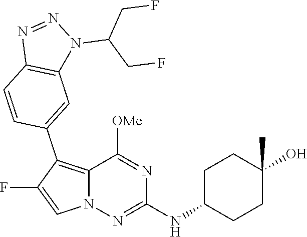 [SMILES: COc1nc(N[C@H]2CC[C@@](C)(O)CC2)nn2cc(F)c(-c3ccc4nnn(C(CF)CF)c4c3)c12] |
| 1504 | 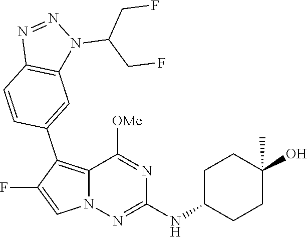 [SMILES: COc1nc(N[C@H]2CC[C@](C)(O)CC2)nn2cc(F)c(-c3ccc4nnn(C(CF)CF)c4c3)c12] |
| 1505 | 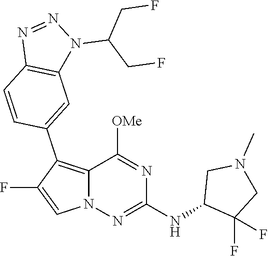 [SMILES: COc1nc(N[C@@H]2CN(C)CC2(F)F)nn2cc(F)c(-c3ccc4nnn(C(CF)CF)c4c3)c12] |
| 1506 | 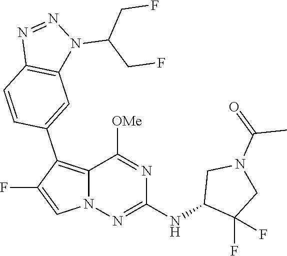 [SMILES: COc1nc(N[C@@H]2CN(C(C)=O)CC2(F)F)nn2cc(F)c(-c3ccc4nnn(C(CF)CF)c4c3)c12] |
| 1507 | 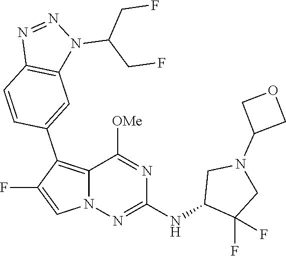 [SMILES: COc1nc(N[C@@H]2CN(C3COC3)CC2(F)F)nn2cc(F)c(-c3ccc4nnn(C(CF)CF)c4c3)c12] |
| 1508 | 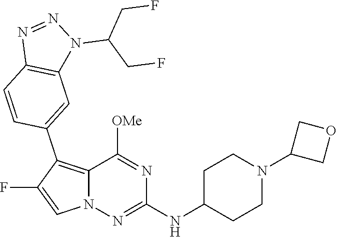 [SMILES: COc1nc(NC2CCN(C3COC3)CC2)nn2cc(F)c(-c3ccc4nnn(C(CF)CF)c4c3)c12] |
| 1509 | 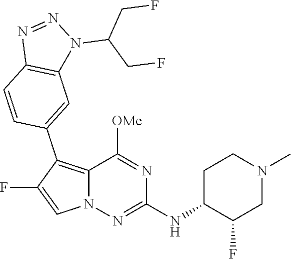 [SMILES: COc1nc(N[C@@H]2CCN(C)C[C@@H]2F)nn2cc(F)c(-c3ccc4nnn(C(CF)CF)c4c3)c12] |
| 1510 | 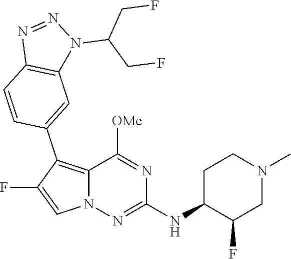 [SMILES: COc1nc(N[C@H]2CCN(C)C[C@H]2F)nn2cc(F)c(-c3ccc4nnn(C(CF)CF)c4c3)c12] |
| 1511 | 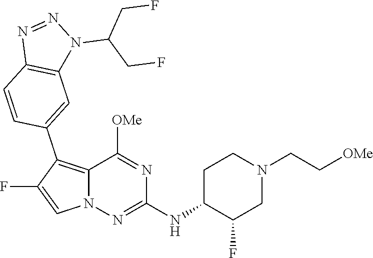 [SMILES: COCCN1CC[C@@H](Nc2nc(OC)c3c(-c4ccc5nnn(C(CF)CF)c5c4)c(F)cn3n2)[C@@H](F)C1] |
| 1512 | 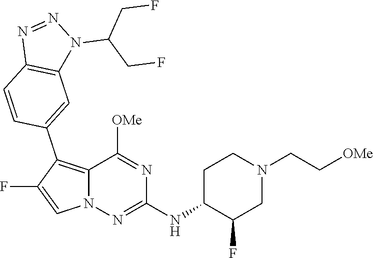 [SMILES: COCCN1CC[C@@H](Nc2nc(OC)c3c(-c4ccc5nnn(C(CF)CF)c5c4)c(F)cn3n2)[C@H](F)C1] |
| 1513 | 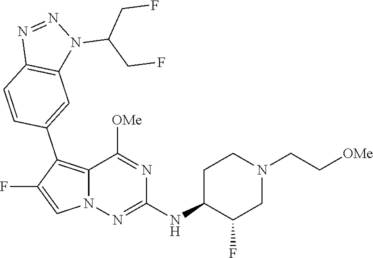 [SMILES: COCCN1CC[C@H](Nc2nc(OC)c3c(-c4ccc5nnn(C(CF)CF)c5c4)c(F)cn3n2)[C@@H](F)C1] |
| 1514 | 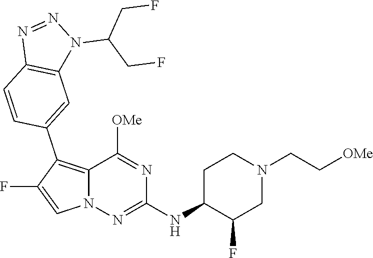 [SMILES: COCCN1CC[C@H](Nc2nc(OC)c3c(-c4ccc5nnn(C(CF)CF)c5c4)c(F)cn3n2)[C@H](F)C1] |
| 1515 | 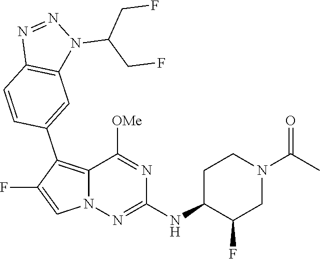 [SMILES: COc1nc(N[C@H]2CCN(C(C)=O)C[C@H]2F)nn2cc(F)c(-c3ccc4nnn(C(CF)CF)c4c3)c12] |
| 1516 | 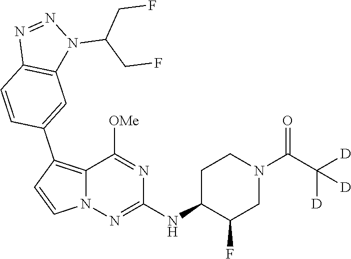 [SMILES: [2H]C([2H])([2H])C(=O)N1CC[C@H](Nc2nc(OC)c3c(-c4ccc5nnn(C(CF)CF)c5c4)ccn3n2)[C@H](F)C1] |
| 1517 | 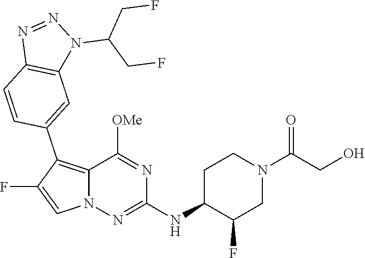 [SMILES: COc1nc(N[C@H]2CCN(C(=O)CO)C[C@H]2F)nn2cc(F)c(-c3ccc4nnn(C(CF)CF)c4c3)c12] |
| 1518 | 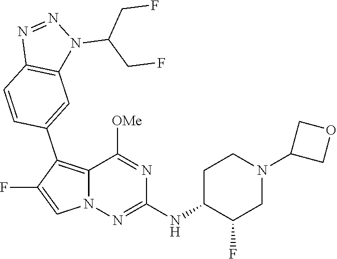 [SMILES: COc1nc(N[C@@H]2CCN(C3COC3)C[C@@H]2F)nn2cc(F)c(-c3ccc4nnn(C(CF)CF)c4c3)c12] |
| 1519 | 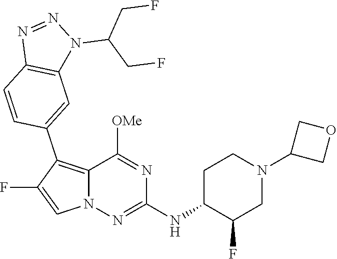 [SMILES: COc1nc(N[C@@H]2CCN(C3COC3)C[C@H]2F)nn2cc(F)c(-c3ccc4nnn(C(CF)CF)c4c3)c12] |
| 1520 | 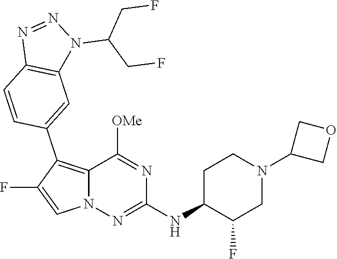 [SMILES: COc1nc(N[C@H]2CCN(C3COC3)C[C@@H]2F)nn2cc(F)c(-c3ccc4nnn(C(CF)CF)c4c3)c12] |
| 1521 | 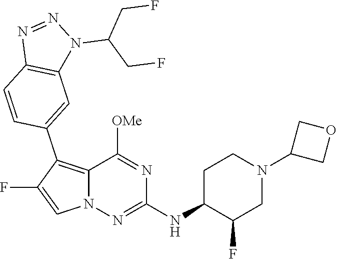 [SMILES: COc1nc(N[C@H]2CCN(C3COC3)C[C@H]2F)nn2cc(F)c(-c3ccc4nnn(C(CF)CF)c4c3)c12] |
| 1522 | 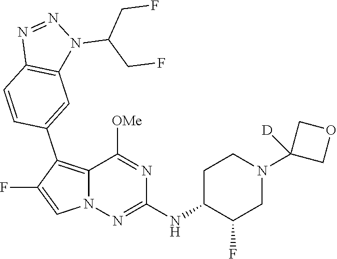 [SMILES: [2H]C1(N2CC[C@@H](Nc3nc(OC)c4c(-c5ccc6nnn(C(CF)CF)c6c5)c(F)cn4n3)[C@@H](F)C2)COC1] |
| 1523 | 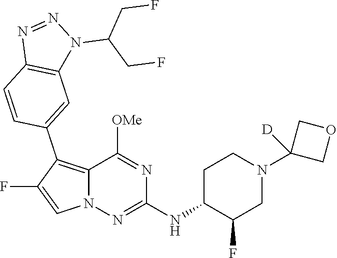 [SMILES: [2H]C1(N2CC[C@@H](Nc3nc(OC)c4c(-c5ccc6nnn(C(CF)CF)c6c5)c(F)cn4n3)[C@H](F)C2)COC1] |
| 1524 | 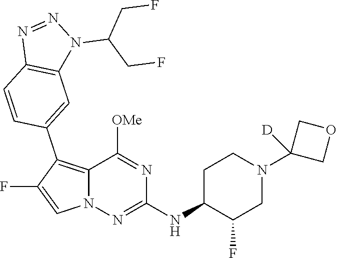 [SMILES: [2H]C1(N2CC[C@H](Nc3nc(OC)c4c(-c5ccc6nnn(C(CF)CF)c6c5)c(F)cn4n3)[C@@H](F)C2)COC1] |
| 1525 | 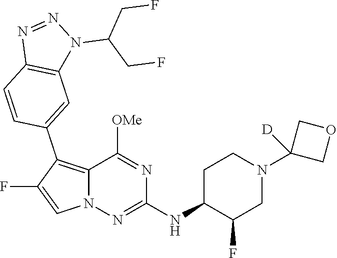 [SMILES: [2H]C1(N2CC[C@H](Nc3nc(OC)c4c(-c5ccc6nnn(C(CF)CF)c6c5)c(F)cn4n3)[C@H](F)C2)COC1] |
| 1526 | 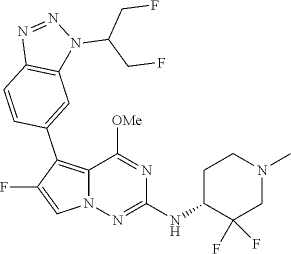 [SMILES: COc1nc(N[C@@H]2CCN(C)CC2(F)F)nn2cc(F)c(-c3ccc4nnn(C(CF)CF)c4c3)c12] |
| 1527 | 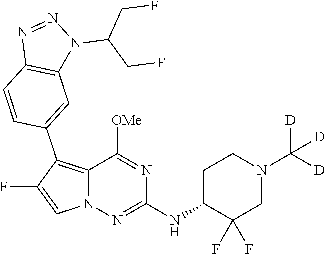 [SMILES: [2H]C([2H])([2H])N1CC[C@@H](Nc2nc(OC)c3c(-c4ccc5nnn(C(CF)CF)c5c4)c(F)cn3n2)C(F)(F)C1] |
| 1528 | 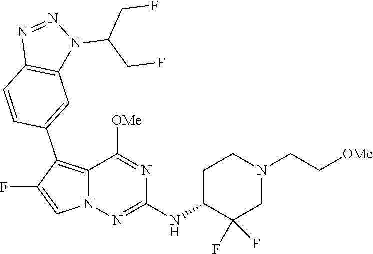 [SMILES: COCCN1CC[C@@H](Nc2nc(OC)c3c(-c4ccc5nnn(C(CF)CF)c5c4)c(F)cn3n2)C(F)(F)C1] |
| 1529 | 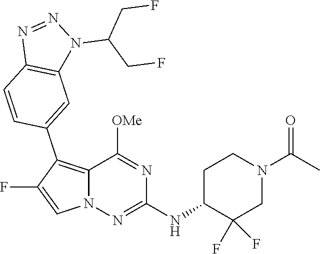 [SMILES: COc1nc(N[C@@H]2CCN(C(C)=O)CC2(F)F)nn2cc(F)c(-c3ccc4nnn(C(CF)CF)c4c3)c12] |
| 1530 | 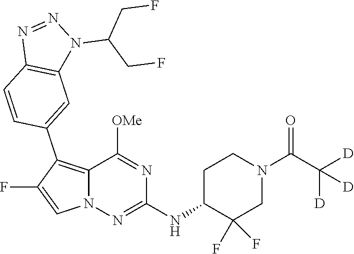 [SMILES: [2H]C([2H])([2H])C(=O)N1CC[C@@H](Nc2nc(OC)c3c(-c4ccc5nnn(C(CF)CF)c5c4)c(F)cn3n2)C(F)(F)C1] |
| 1531 | 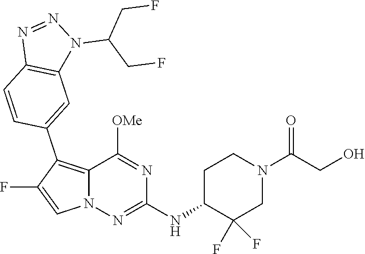 [SMILES: COc1nc(N[C@@H]2CCN(C(=O)CO)CC2(F)F)nn2cc(F)c(-c3ccc4nnn(C(CF)CF)c4c3)c12] |
| 1532 | 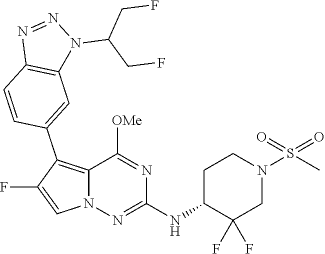 [SMILES: COc1nc(N[C@@H]2CCN(S(C)(=O)=O)CC2(F)F)nn2cc(F)c(-c3ccc4nnn(C(CF)CF)c4c3)c12] |
| 1533 | 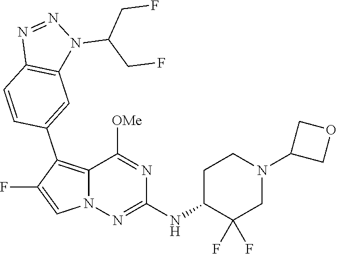 [SMILES: COc1nc(N[C@@H]2CCN(C3COC3)CC2(F)F)nn2cc(F)c(-c3ccc4nnn(C(CF)CF)c4c3)c12] |
| 1534 | 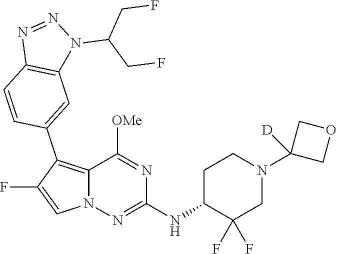 [SMILES: [2H]C1(N2CC[C@@H](Nc3nc(OC)c4c(-c5ccc6nnn(C(CF)CF)c6c5)c(F)cn4n3)C(F)(F)C2)COC1] |
| 1535 | 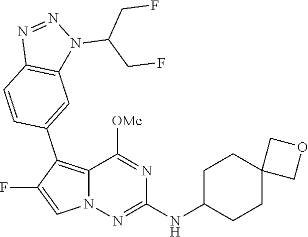 [SMILES: COc1nc(NC2CCC3(CC2)COC3)nn2cc(F)c(-c3ccc4nnn(C(CF)CF)c4c3)c12] |
| 1536 | 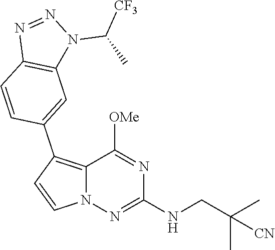 [SMILES: COc1nc(NCC(C)(C)C#N)nn2ccc(-c3ccc4nnn([C@@H](C)C(F)(F)F)c4c3)c12] |
| 1537 | 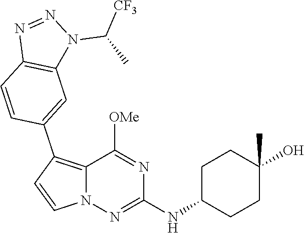 [SMILES: COc1nc(N[C@H]2CC[C@@](C)(O)CC2)nn2ccc(-c3ccc4nnn([C@@H](C)C(F)(F)F)c4c3)c12] |
| 1538 | 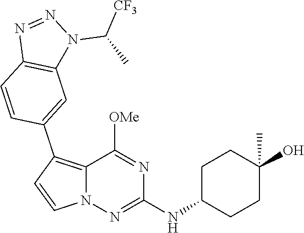 [SMILES: COc1nc(N[C@H]2CC[C@](C)(O)CC2)nn2ccc(-c3ccc4nnn([C@@H](C)C(F)(F)F)c4c3)c12] |
| 1539 | 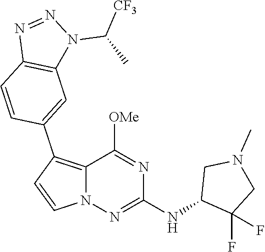 [SMILES: COc1nc(N[C@@H]2CN(C)CC2(F)F)nn2ccc(-c3ccc4nnn([C@@H](C)C(F)(F)F)c4c3)c12] |
| 1540 | 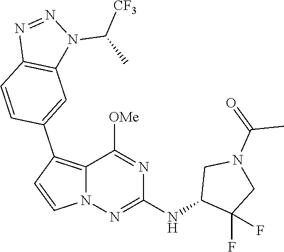 [SMILES: COc1nc(N[C@@H]2CN(C(C)=O)CC2(F)F)nn2ccc(-c3ccc4nnn([C@@H](C)C(F)(F)F)c4c3)c12] |
| 1541 | 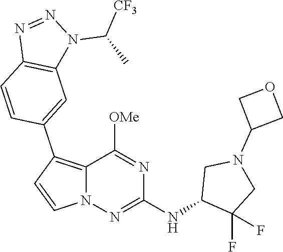 [SMILES: COc1nc(N[C@@H]2CN(C3COC3)CC2(F)F)nn2ccc(-c3ccc4nnn([C@@H](C)C(F)(F)F)c4c3)c12] |
| 1542 | 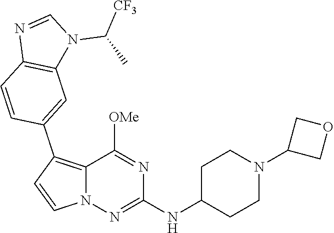 [SMILES: COc1nc(NC2CCN(C3COC3)CC2)nn2ccc(-c3ccc4ncn([C@@H](C)C(F)(F)F)c4c3)c12] |
| 1543 | 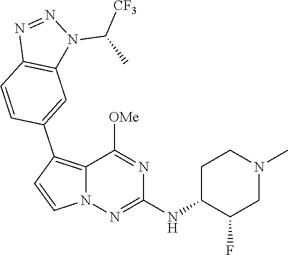 [SMILES: COc1nc(N[C@@H]2CCN(C)C[C@@H]2F)nn2ccc(-c3ccc4nnn([C@@H](C)C(F)(F)F)c4c3)c12] |
| 1544 | 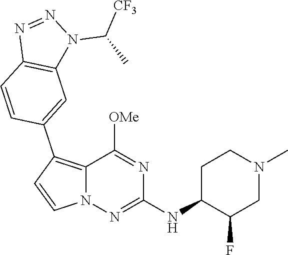 [SMILES: COc1nc(N[C@H]2CCN(C)C[C@H]2F)nn2ccc(-c3ccc4nnn([C@@H](C)C(F)(F)F)c4c3)c12] |
| 1545 | 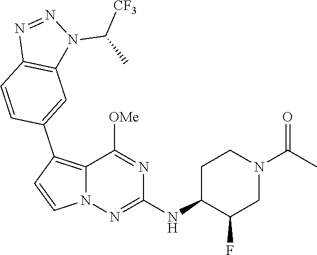 [SMILES: COc1nc(N[C@H]2CCN(C(C)=O)C[C@H]2F)nn2ccc(-c3ccc4nnn([C@@H](C)C(F)(F)F)c4c3)c12] |
| 1546 | 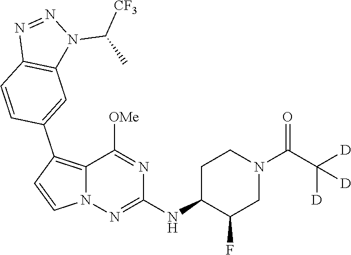 [SMILES: [2H]C([2H])([2H])C(=O)N1CC[C@H](Nc2nc(OC)c3c(-c4ccc5nnn([C@@H](C)C(F)(F)F)c5c4)ccn3n2)[C@H](F)C1] |
| 1547 | 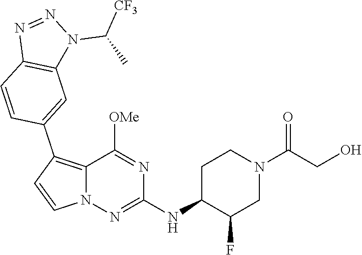 [SMILES: COc1nc(N[C@H]2CCN(C(=O)CO)C[C@H]2F)nn2ccc(-c3ccc4nnn([C@@H](C)C(F)(F)F)c4c3)c12] |
| 1548 | 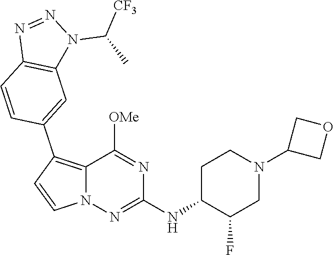 [SMILES: COc1nc(N[C@@H]2CCN(C3COC3)C[C@@H]2F)nn2ccc(-c3ccc4nnn([C@@H](C)C(F)(F)F)c4c3)c12] |
| 1549 | 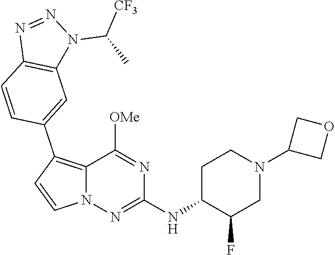 [SMILES: COc1nc(N[C@@H]2CCN(C3COC3)C[C@H]2F)nn2ccc(-c3ccc4nnn([C@@H](C)C(F)(F)F)c4c3)c12] |
| 1550 | 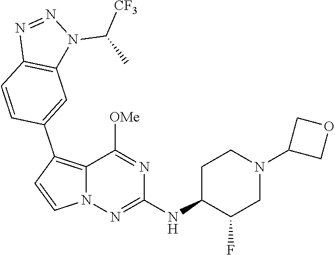 [SMILES: COc1nc(N[C@H]2CCN(C3COC3)C[C@@H]2F)nn2ccc(-c3ccc4nnn([C@@H](C)C(F)(F)F)c4c3)c12] |
| 1551 | 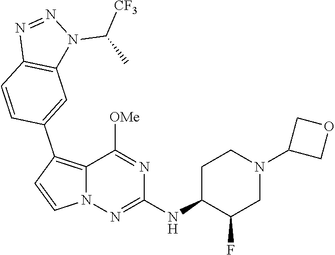 [SMILES: COc1nc(N[C@H]2CCN(C3COC3)C[C@H]2F)nn2ccc(-c3ccc4nnn([C@@H](C)C(F)(F)F)c4c3)c12] |
| 1552 | 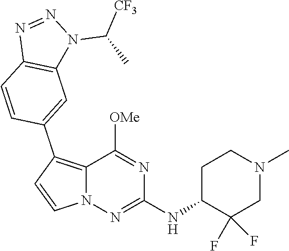 [SMILES: COc1nc(N[C@@H]2CCN(C)CC2(F)F)nn2ccc(-c3ccc4nnn([C@@H](C)C(F)(F)F)c4c3)c12] |
| 1553 | 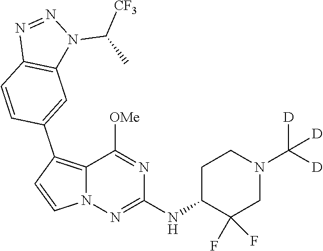 [SMILES: [2H]C([2H])([2H])N1CC[C@@H](Nc2nc(OC)c3c(-c4ccc5nnn([C@@H](C)C(F)(F)F)c5c4)ccn3n2)C(F)(F)C1] |
| 1554 | 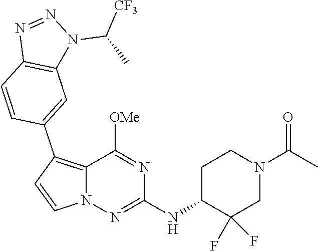 [SMILES: COc1nc(N[C@@H]2CCN(C(C)=O)CC2(F)F)nn2ccc(-c3ccc4nnn([C@@H](C)C(F)(F)F)c4c3)c12] |
| 1555 | 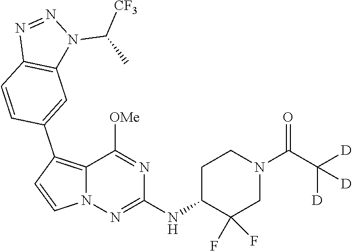 [SMILES: [2H]C([2H])([2H])C(=O)N1CC[C@@H](Nc2nc(OC)c3c(-c4ccc5nnn([C@@H](C)C(F)(F)F)c5c4)ccn3n2)C(F)(F)C1] |
| 1556 | 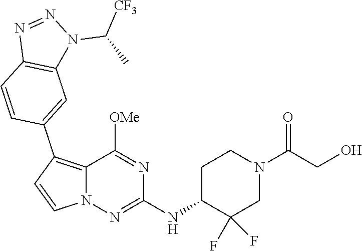 [SMILES: COc1nc(N[C@@H]2CCN(C(=O)CO)CC2(F)F)nn2ccc(-c3ccc4nnn([C@@H](C)C(F)(F)F)c4c3)c12] |
| 1557 | 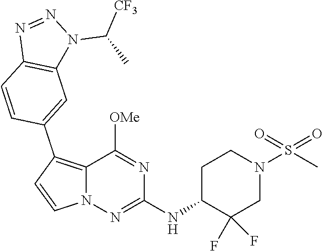 [SMILES: COc1nc(N[C@@H]2CCN(S(C)(=O)=O)CC2(F)F)nn2ccc(-c3ccc4nnn([C@@H](C)C(F)(F)F)c4c3)c12] |
| 1558 | 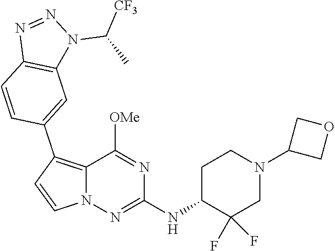 [SMILES: COc1nc(N[C@@H]2CCN(C3COC3)CC2(F)F)nn2ccc(-c3ccc4nnn([C@@H](C)C(F)(F)F)c4c3)c12] |
| 1559 | 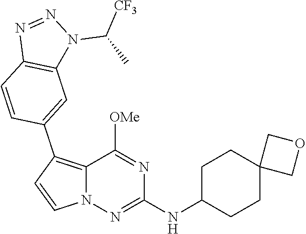 [SMILES: COc1nc(NC2CCC3(CC2)COC3)nn2ccc(-c3ccc4nnn([C@@H](C)C(F)(F)F)c4c3)c12] |
| 1560 | 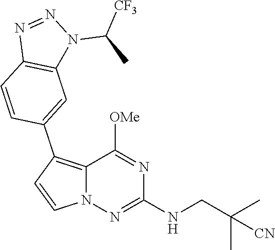 [SMILES: COc1nc(NCC(C)(C)C#N)nn2ccc(-c3ccc4nnn([C@H](C)C(F)(F)F)c4c3)c12] |
| 1561 | 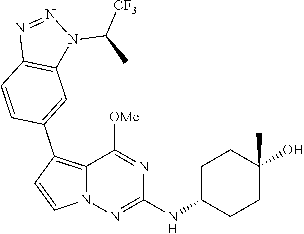 [SMILES: COc1nc(N[C@H]2CC[C@@](C)(O)CC2)nn2ccc(-c3ccc4nnn([C@H](C)C(F)(F)F)c4c3)c12] |
| 1562 | 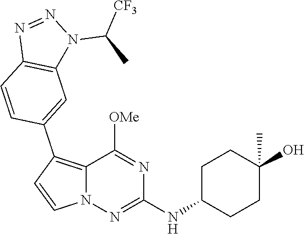 [SMILES: COc1nc(N[C@H]2CC[C@](C)(O)CC2)nn2ccc(-c3ccc4nnn([C@H](C)C(F)(F)F)c4c3)c12] |
| 1563 | 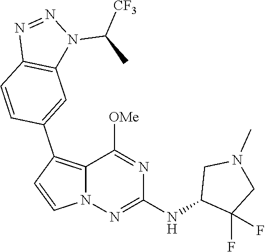 [SMILES: COc1nc(N[C@@H]2CN(C)CC2(F)F)nn2ccc(-c3ccc4nnn([C@H](C)C(F)(F)F)c4c3)c12] |
| 1564 | 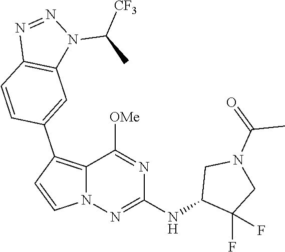 [SMILES: COc1nc(N[C@@H]2CN(C(C)=O)CC2(F)F)nn2ccc(-c3ccc4nnn([C@H](C)C(F)(F)F)c4c3)c12] |
| 1565 | 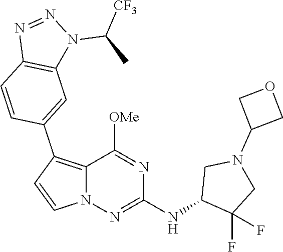 [SMILES: COc1nc(N[C@@H]2CN(C3COC3)CC2(F)F)nn2ccc(-c3ccc4nnn([C@H](C)C(F)(F)F)c4c3)c12] |
| 1566 | 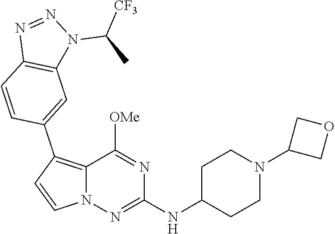 [SMILES: COc1nc(NC2CCN(C3COC3)CC2)nn2ccc(-c3ccc4nnn([C@H](C)C(F)(F)F)c4c3)c12] |
| 1567 | 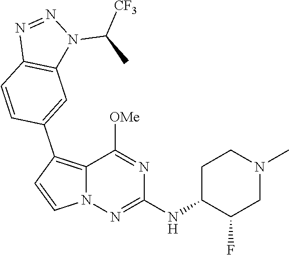 [SMILES: COc1nc(N[C@@H]2CCN(C)C[C@@H]2F)nn2ccc(-c3ccc4nnn([C@H](C)C(F)(F)F)c4c3)c12] |
| 1568 | 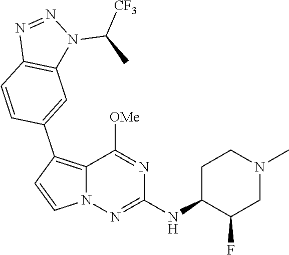 [SMILES: COc1nc(N[C@H]2CCN(C)C[C@H]2F)nn2ccc(-c3ccc4nnn([C@H](C)C(F)(F)F)c4c3)c12] |
| 1569 | 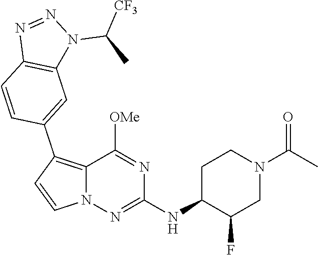 [SMILES: COc1nc(N[C@H]2CCN(C(C)=O)C[C@H]2F)nn2ccc(-c3ccc4nnn([C@H](C)C(F)(F)F)c4c3)c12] |
| 1570 | 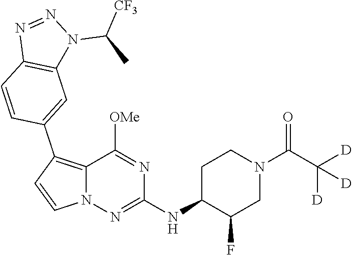 [SMILES: [2H]C([2H])([2H])C(=O)N1CC[C@H](Nc2nc(OC)c3c(-c4ccc5nnn([C@H](C)C(F)(F)F)c5c4)ccn3n2)[C@H](F)C1] |
| 1571 | 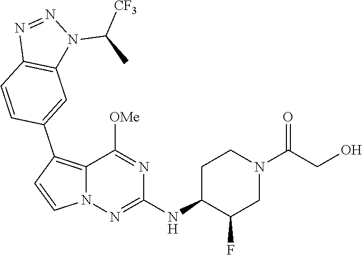 [SMILES: COc1nc(N[C@H]2CCN(C(=O)CO)C[C@H]2F)nn2ccc(-c3ccc4nnn([C@H](C)C(F)(F)F)c4c3)c12] |
| 1572 | 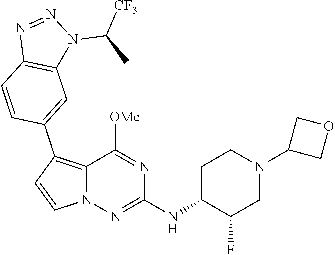 [SMILES: COc1nc(N[C@@H]2CCN(C3COC3)C[C@@H]2F)nn2ccc(-c3ccc4nnn([C@H](C)C(F)(F)F)c4c3)c12] |
| 1573 | 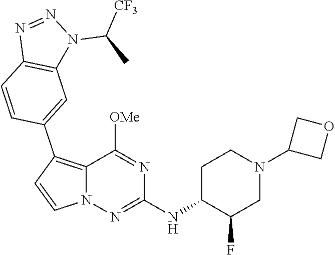 [SMILES: COc1nc(N[C@@H]2CCN(C3COC3)C[C@H]2F)nn2ccc(-c3ccc4nnn([C@H](C)C(F)(F)F)c4c3)c12] |
| 1574 | 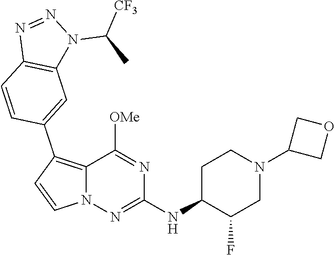 [SMILES: COc1nc(N[C@H]2CCN(C3COC3)C[C@@H]2F)nn2ccc(-c3ccc4nnn([C@H](C)C(F)(F)F)c4c3)c12] |
| 1575 | 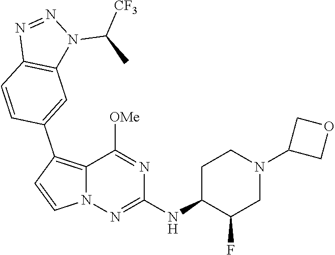 [SMILES: COc1nc(N[C@H]2CCN(C3COC3)C[C@H]2F)nn2ccc(-c3ccc4nnn([C@H](C)C(F)(F)F)c4c3)c12] |
| 1576 | 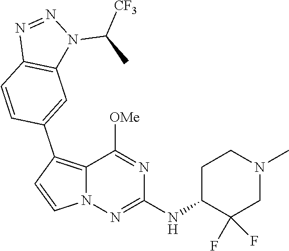 [SMILES: COc1nc(N[C@@H]2CCN(C)CC2(F)F)nn2ccc(-c3ccc4nnn([C@H](C)C(F)(F)F)c4c3)c12] |
| 1577 | 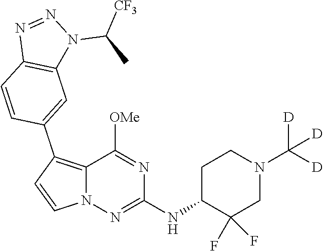 [SMILES: [2H]C([2H])([2H])N1CC[C@@H](Nc2nc(OC)c3c(-c4ccc5nnn([C@H](C)C(F)(F)F)c5c4)ccn3n2)C(F)(F)C1] |
| 1578 | 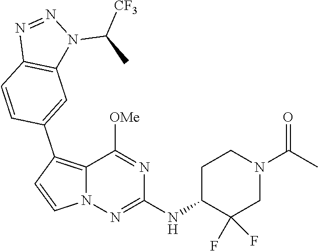 [SMILES: COc1nc(N[C@@H]2CCN(C(C)=O)CC2(F)F)nn2ccc(-c3ccc4nnn([C@H](C)C(F)(F)F)c4c3)c12] |
| 1579 | 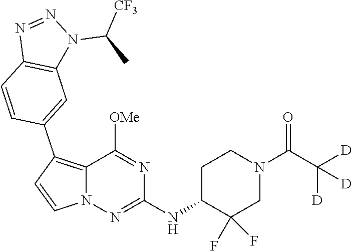 [SMILES: [2H]C([2H])([2H])C(=O)N1CC[C@@H](Nc2nc(OC)c3c(-c4ccc5nnn([C@H](C)C(F)(F)F)c5c4)ccn3n2)C(F)(F)C1] |
| 1580 | 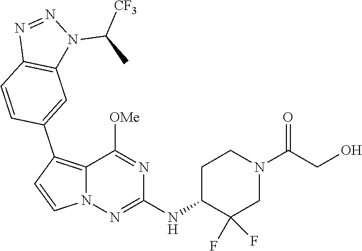 [SMILES: COc1nc(N[C@@H]2CCN(C(=O)CO)CC2(F)F)nn2ccc(-c3ccc4nnn([C@H](C)C(F)(F)F)c4c3)c12] |
| 1581 | 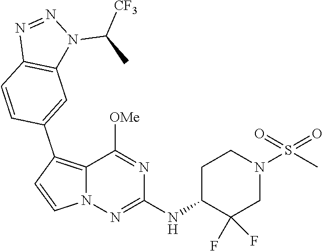 [SMILES: COc1nc(N[C@@H]2CCN(S(C)(=O)=O)CC2(F)F)nn2ccc(-c3ccc4nnn([C@H](C)C(F)(F)F)c4c3)c12] |
| 1582 | 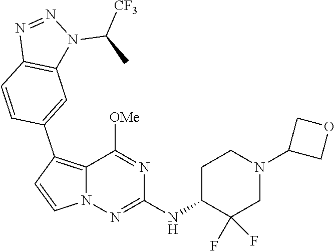 [SMILES: COc1nc(N[C@@H]2CCN(C3COC3)CC2(F)F)nn2ccc(-c3ccc4nnn([C@H](C)C(F)(F)F)c4c3)c12] |
| 1583 | 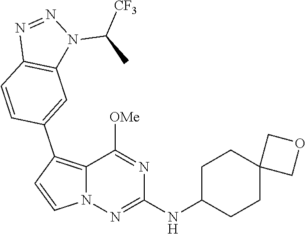 [SMILES: COc1nc(NC2CCC3(CC2)COC3)nn2ccc(-c3ccc4nnn([C@H](C)C(F)(F)F)c4c3)c12] |
| 1584 | 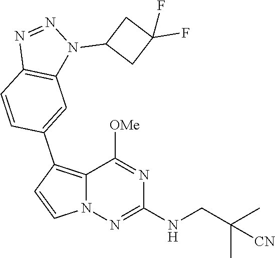 [SMILES: COc1nc(NCC(C)(C)C#N)nn2ccc(-c3ccc4nnn(C5CC(F)(F)C5)c4c3)c12] |
| 1585 | 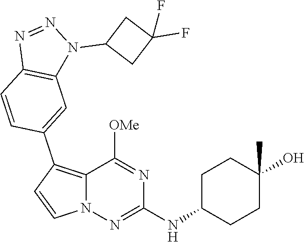 [SMILES: COc1nc(N[C@H]2CC[C@@](C)(O)CC2)nn2ccc(-c3ccc4nnn(C5CC(F)(F)C5)c4c3)c12] |
| 1586 | 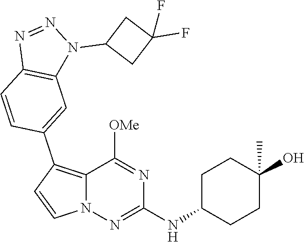 [SMILES: COc1nc(N[C@H]2CC[C@](C)(O)CC2)nn2ccc(-c3ccc4nnn(C5CC(F)(F)C5)c4c3)c12] |
| 1587 | 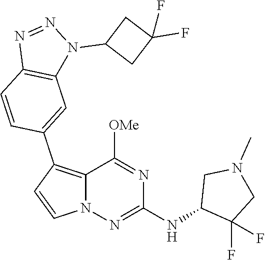 [SMILES: COc1nc(N[C@@H]2CN(C)CC2(F)F)nn2ccc(-c3ccc4nnn(C5CC(F)(F)C5)c4c3)c12] |
| 1588 | 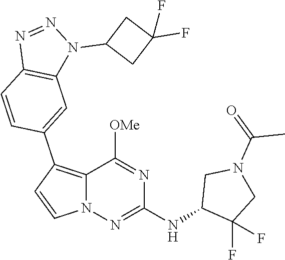 [SMILES: COc1nc(N[C@@H]2CN(C(C)=O)CC2(F)F)nn2ccc(-c3ccc4nnn(C5CC(F)(F)C5)c4c3)c12] |
| 1589 | 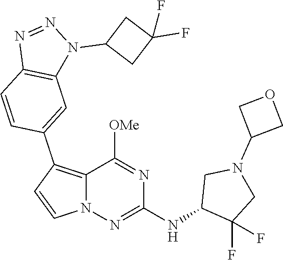 [SMILES: COc1nc(N[C@@H]2CN(C3COC3)CC2(F)F)nn2ccc(-c3ccc4nnn(C5CC(F)(F)C5)c4c3)c12] |
| 1590 | 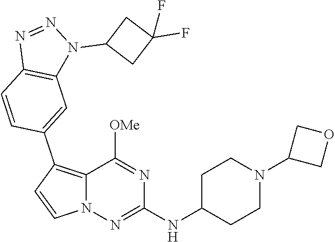 [SMILES: COc1nc(NC2CCN(C3COC3)CC2)nn2ccc(-c3ccc4nnn(C5CC(F)(F)C5)c4c3)c12] |
| 1591 | 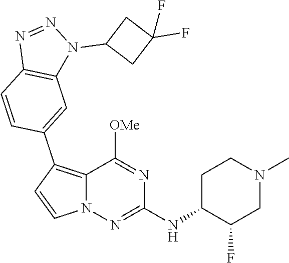 [SMILES: COc1nc(N[C@@H]2CCN(C)C[C@@H]2F)nn2ccc(-c3ccc4nnn(C5CC(F)(F)C5)c4c3)c12] |
| 1592 | 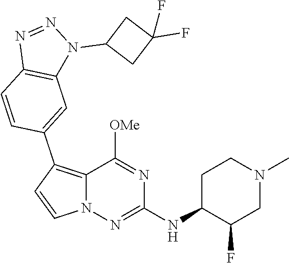 [SMILES: COc1nc(N[C@H]2CCN(C)C[C@H]2F)nn2ccc(-c3ccc4nnn(C5CC(F)(F)C5)c4c3)c12] |
| 1593 | 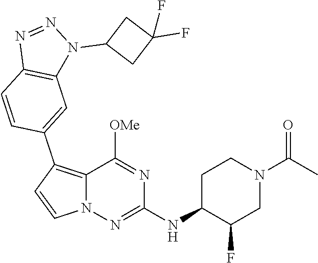 [SMILES: COc1nc(N[C@H]2CCN(C(C)=O)C[C@H]2F)nn2ccc(-c3ccc4nnn(C5CC(F)(F)C5)c4c3)c12] |
| 1594 | 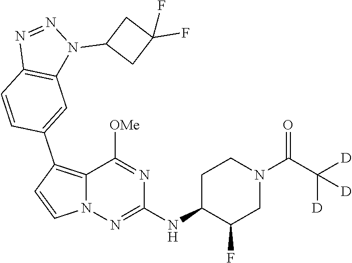 [SMILES: [2H]C([2H])([2H])C(=O)N1CC[C@H](Nc2nc(OC)c3c(-c4ccc5nnn(C6CC(F)(F)C6)c5c4)ccn3n2)[C@H](F)C1] |
| 1595 | 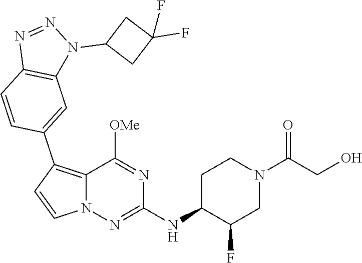 [SMILES: COc1nc(N[C@H]2CCN(C(=O)CO)C[C@H]2F)nn2ccc(-c3ccc4nnn(C5CC(F)(F)C5)c4c3)c12] |
| 1596 | 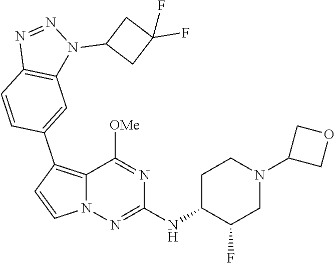 [SMILES: COc1nc(N[C@@H]2CCN(C3COC3)C[C@@H]2F)nn2ccc(-c3ccc4nnn(C5CC(F)(F)C5)c4c3)c12] |
| 1597 | 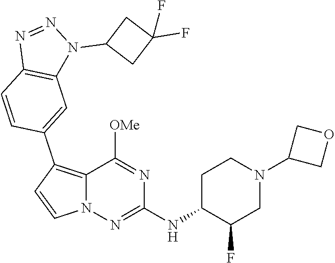 [SMILES: COc1nc(N[C@@H]2CCN(C3COC3)C[C@H]2F)nn2ccc(-c3ccc4nnn(C5CC(F)(F)C5)c4c3)c12] |
| 1598 | 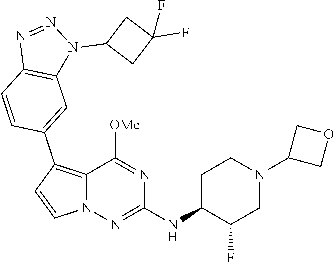 [SMILES: COc1nc(N[C@H]2CCN(C3COC3)C[C@@H]2F)nn2ccc(-c3ccc4nnn(C5CC(F)(F)C5)c4c3)c12] |
| 1599 | 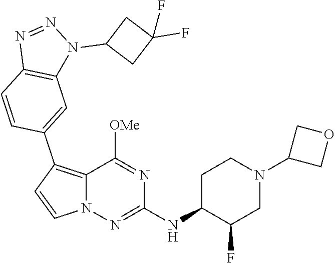 [SMILES: COc1nc(N[C@H]2CCN(C3COC3)C[C@H]2F)nn2ccc(-c3ccc4nnn(C5CC(F)(F)C5)c4c3)c12] |
| 1600 | 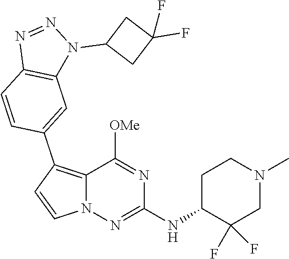 [SMILES: COc1nc(N[C@@H]2CCN(C)CC2(F)F)nn2ccc(-c3ccc4nnn(C5CC(F)(F)C5)c4c3)c12] |
| 1601 | 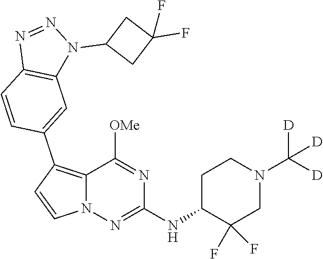 [SMILES: [2H]C([2H])([2H])N1CC[C@@H](Nc2nc(OC)c3c(-c4ccc5nnn(C6CC(F)(F)C6)c5c4)ccn3n2)C(F)(F)C1] |
| 1602 | 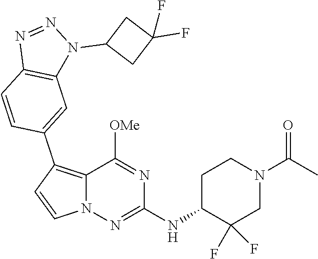 [SMILES: COc1nc(N[C@@H]2CCN(C(C)=O)CC2(F)F)nn2ccc(-c3ccc4nnn(C5CC(F)(F)C5)c4c3)c12] |
| 1603 | 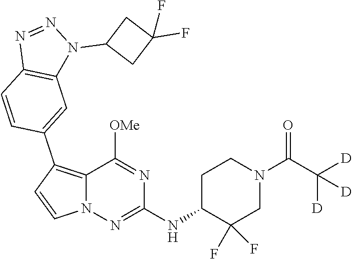 [SMILES: [2H]C([2H])([2H])C(=O)N1CC[C@@H](Nc2nc(OC)c3c(-c4ccc5nnn(C6CC(F)(F)C6)c5c4)ccn3n2)C(F)(F)C1] |
| 1604 | 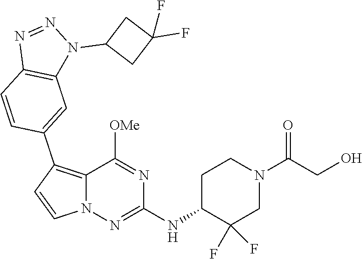 [SMILES: COc1nc(N[C@@H]2CCN(C(=O)CO)CC2(F)F)nn2ccc(-c3ccc4nnn(C5CC(F)(F)C5)c4c3)c12] |
| 1605 | 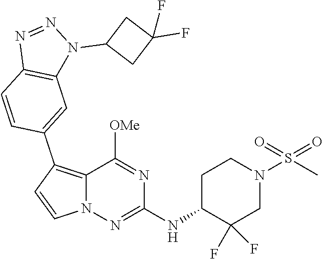 [SMILES: COc1nc(N[C@@H]2CCN(S(C)(=O)=O)CC2(F)F)nn2ccc(-c3ccc4nnn(C5CC(F)(F)C5)c4c3)c12] |
| 1606 | 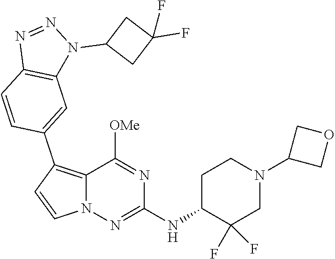 [SMILES: COc1nc(N[C@@H]2CCN(C3COC3)CC2(F)F)nn2ccc(-c3ccc4nnn(C5CC(F)(F)C5)c4c3)c12] |
| 1607 | 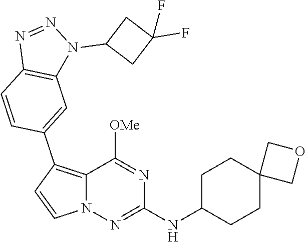 [SMILES: COc1nc(NC2CCC3(CC2)COC3)nn2ccc(-c3ccc4nnn(C5CC(F)(F)C5)c4c3)c12] |
| 1608 | 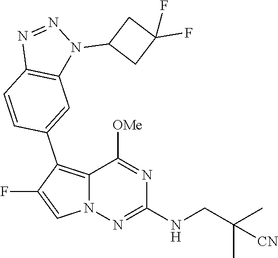 [SMILES: COc1nc(NCC(C)(C)C#N)nn2cc(F)c(-c3ccc4nnn(C5CC(F)(F)C5)c4c3)c12] |
| 1609 | 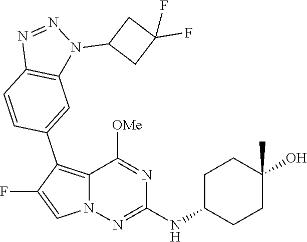 [SMILES: COc1nc(N[C@H]2CC[C@@](C)(O)CC2)nn2cc(F)c(-c3ccc4nnn(C5CC(F)(F)C5)c4c3)c12] |
| 1610 | 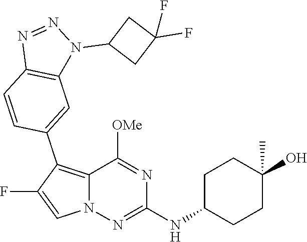 [SMILES: COc1nc(N[C@H]2CC[C@](C)(O)CC2)nn2cc(F)c(-c3ccc4nnn(C5CC(F)(F)C5)c4c3)c12] |
| 1611 | 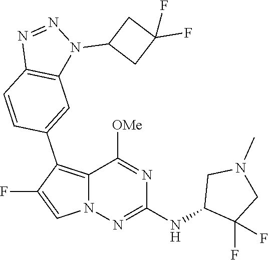 [SMILES: COc1nc(N[C@@H]2CN(C)CC2(F)F)nn2cc(F)c(-c3ccc4nnn(C5CC(F)(F)C5)c4c3)c12] |
| 1612 | 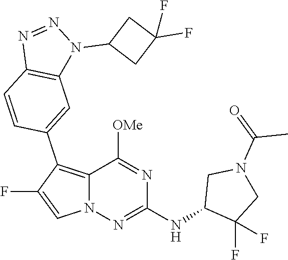 [SMILES: COc1nc(N[C@@H]2CN(C(C)=O)CC2(F)F)nn2cc(F)c(-c3ccc4nnn(C5CC(F)(F)C5)c4c3)c12] |
| 1613 | 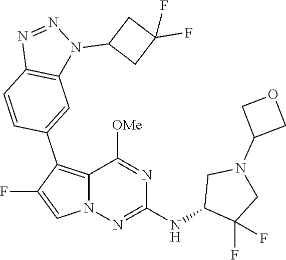 [SMILES: COc1nc(N[C@@H]2CN(C3COC3)CC2(F)F)nn2cc(F)c(-c3ccc4nnn(C5CC(F)(F)C5)c4c3)c12] |
| 1614 | 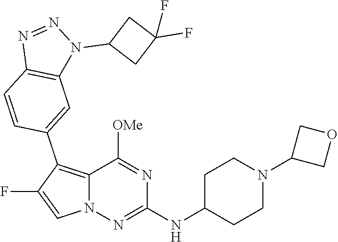 [SMILES: COc1nc(NC2CCN(C3COC3)CC2)nn2cc(F)c(-c3ccc4nnn(C5CC(F)(F)C5)c4c3)c12] |
| 1615 | 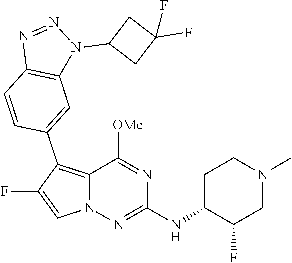 [SMILES: COc1nc(N[C@@H]2CCN(C)C[C@@H]2F)nn2cc(F)c(-c3ccc4nnn(C5CC(F)(F)C5)c4c3)c12] |
| 1616 | 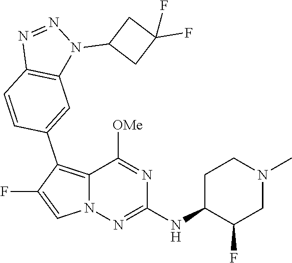 [SMILES: COc1nc(N[C@H]2CCN(C)C[C@H]2F)nn2cc(F)c(-c3ccc4nnn(C5CC(F)(F)C5)c4c3)c12] |
| 1617 | 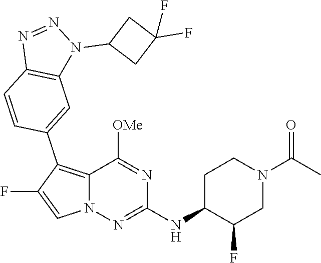 [SMILES: COc1nc(N[C@H]2CCN(C(C)=O)C[C@H]2F)nn2cc(F)c(-c3ccc4nnn(C5CC(F)(F)C5)c4c3)c12] |
| 1618 | 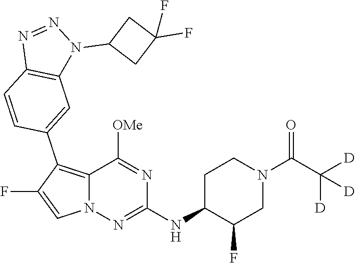 [SMILES: [2H]C([2H])([2H])C(=O)N1CC[C@H](Nc2nc(OC)c3c(-c4ccc5nnn(C6CC(F)(F)C6)c5c4)c(F)cn3n2)[C@H](F)C1] |
| 1619 | 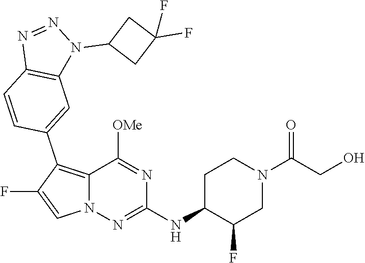 [SMILES: COc1nc(N[C@H]2CCN(C(=O)CO)C[C@H]2F)nn2cc(F)c(-c3ccc4nnn(C5CC(F)(F)C5)c4c3)c12] |
| 1620 | 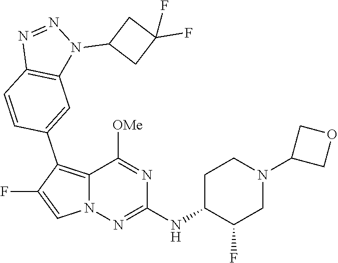 [SMILES: COc1nc(N[C@@H]2CCN(C3COC3)C[C@@H]2F)nn2cc(F)c(-c3ccc4nnn(C5CC(F)(F)C5)c4c3)c12] |
| 1621 | 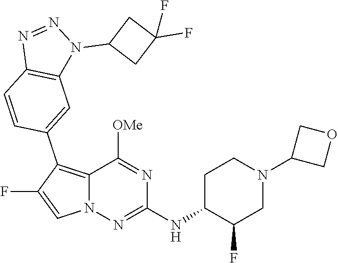 [SMILES: COc1nc(N[C@@H]2CCN(C3COC3)C[C@H]2F)nn2cc(F)c(-c3ccc4nnn(C5CC(F)(F)C5)c4c3)c12] |
| 1622 | 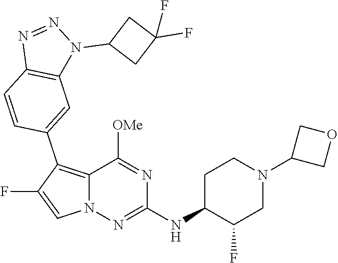 [SMILES: COc1nc(N[C@H]2CCN(C3COC3)C[C@@H]2F)nn2cc(F)c(-c3ccc4nnn(C5CC(F)(F)C5)c4c3)c12] |
| 1623 | 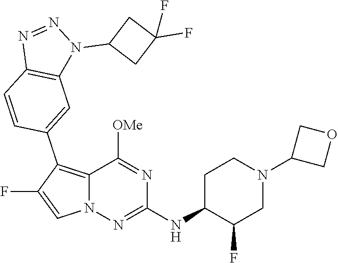 [SMILES: COc1nc(N[C@H]2CCN(C3COC3)C[C@H]2F)nn2cc(F)c(-c3ccc4nnn(C5CC(F)(F)C5)c4c3)c12] |
| 1624 | 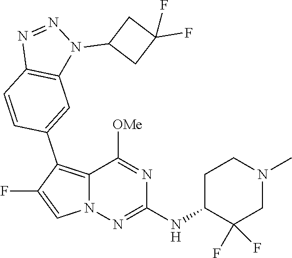 [SMILES: COc1nc(N[C@@H]2CCN(C)CC2(F)F)nn2cc(F)c(-c3ccc4nnn(C5CC(F)(F)C5)c4c3)c12] |
| 1625 | 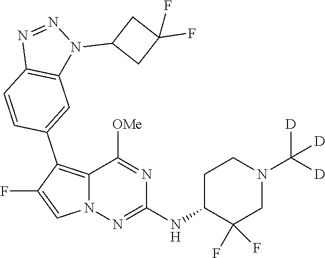 [SMILES: [2H]C([2H])([2H])N1CC[C@@H](Nc2nc(OC)c3c(-c4ccc5nnn(C6CC(F)(F)C6)c5c4)c(F)cn3n2)C(F)(F)C1] |
| 1626 | 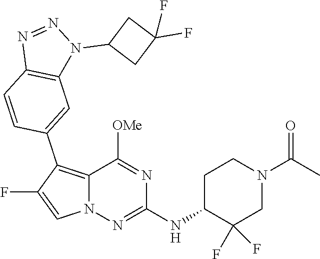 [SMILES: COc1nc(N[C@@H]2CCN(C(C)=O)CC2(F)F)nn2cc(F)c(-c3ccc4nnn(C5CC(F)(F)C5)c4c3)c12] |
| 1627 | 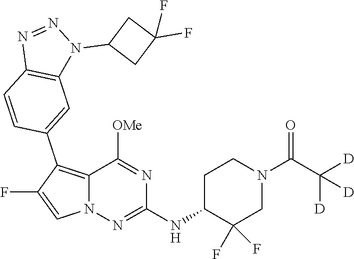 [SMILES: [2H]C([2H])([2H])C(=O)N1CC[C@@H](Nc2nc(OC)c3c(-c4ccc5nnn(C6CC(F)(F)C6)c5c4)c(F)cn3n2)C(F)(F)C1] |
| 1628 | 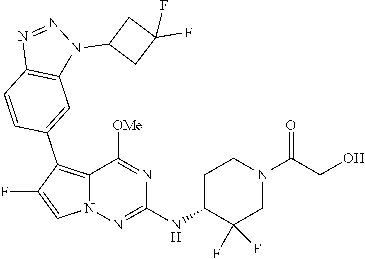 [SMILES: COc1nc(N[C@@H]2CCN(C(=O)CO)CC2(F)F)nn2cc(F)c(-c3ccc4nnn(C5CC(F)(F)C5)c4c3)c12] |
| 1629 | 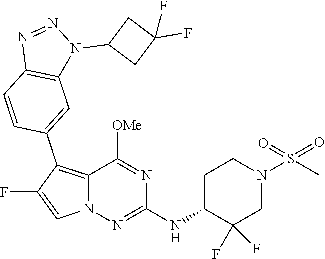 [SMILES: COc1nc(N[C@@H]2CCN(S(C)(=O)=O)CC2(F)F)nn2cc(F)c(-c3ccc4nnn(C5CC(F)(F)C5)c4c3)c12] |
| 1630 | 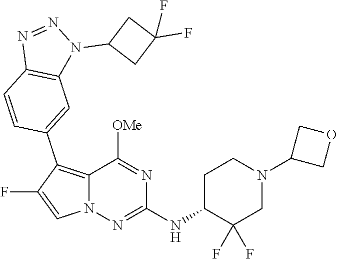 [SMILES: COc1nc(N[C@@H]2CCN(C3COC3)CC2(F)F)nn2cc(F)c(-c3ccc4nnn(C5CC(F)(F)C5)c4c3)c12] |
| 1631 | 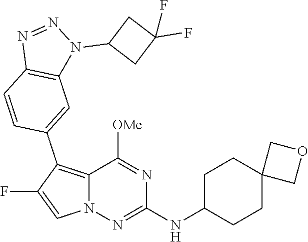 [SMILES: COc1nc(NC2CCC3(CC2)COC3)nn2cc(F)c(-c3ccc4nnn(C5CC(F)(F)C5)c4c3)c12] |
| 1632 | 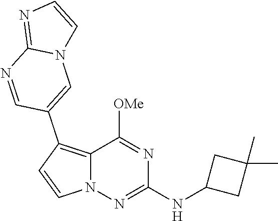 [SMILES: COc1nc(NC2CC(C)(C)C2)nn2ccc(-c3cnc4nccn4c3)c12] |
| 1633 | 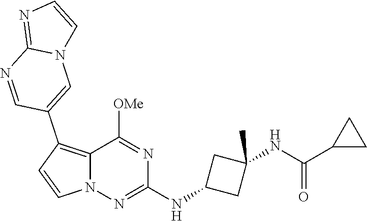 [SMILES: COc1nc(N[C@H]2C[C@@](C)(NC(=O)C3CC3)C2)nn2ccc(-c3cnc4nccn4c3)c12] |
| 1634 | 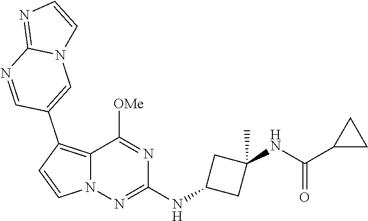 [SMILES: COc1nc(N[C@H]2C[C@](C)(NC(=O)C3CC3)C2)nn2ccc(-c3cnc4nccn4c3)c12] |
| 1635 | 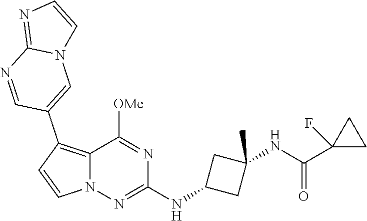 [SMILES: COc1nc(N[C@H]2C[C@@](C)(NC(=O)C3(F)CC3)C2)nn2ccc(-c3cnc4nccn4c3)c12] |
| 1636 | 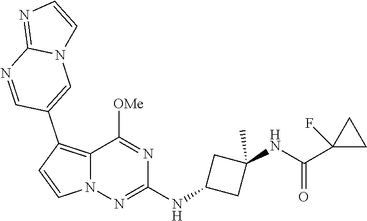 [SMILES: COc1nc(N[C@H]2C[C@](C)(NC(=O)C3(F)CC3)C2)nn2ccc(-c3cnc4nccn4c3)c12] |
| 1637 | 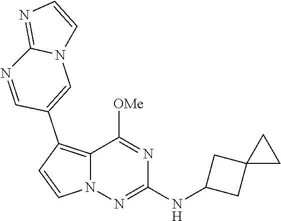 [SMILES: COc1nc(NC2CC3(CC3)C2)nn2ccc(-c3cnc4nccn4c3)c12] |
| 1638 | 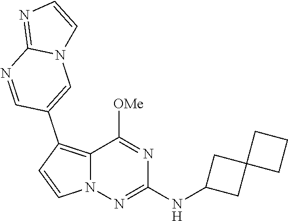 [SMILES: COc1nc(NC2CC3(CCC3)C2)nn2ccc(-c3cnc4nccn4c3)c12] |
| 1639 | 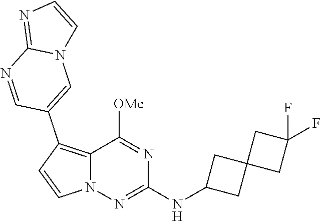 [SMILES: COc1nc(NC2CC3(C2)CC(F)(F)C3)nn2ccc(-c3cnc4nccn4c3)c12] |
| 1640 | 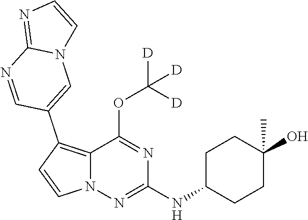 [SMILES: [2H]C([2H])([2H])Oc1nc(N[C@H]2CC[C@](C)(O)CC2)nn2ccc(-c3cnc4nccn4c3)c12] |
| 1641 | 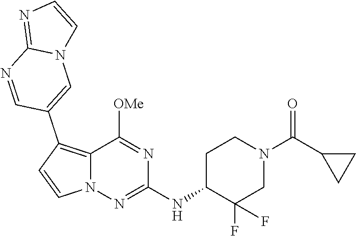 [SMILES: COc1nc(N[C@@H]2CCN(C(=O)C3CC3)CC2(F)F)nn2ccc(-c3cnc4nccn4c3)c12] |
| 1642 | 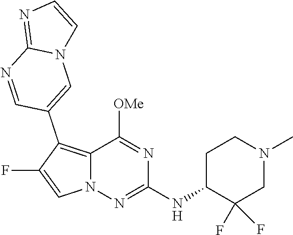 [SMILES: COc1nc(N[C@@H]2CCN(C)CC2(F)F)nn2cc(F)c(-c3cnc4nccn4c3)c12] |
| 1643 | 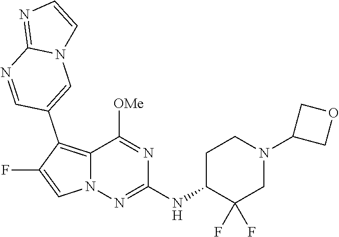 [SMILES: COc1nc(N[C@@H]2CCN(C3COC3)CC2(F)F)nn2cc(F)c(-c3cnc4nccn4c3)c12] |
| 1644 | 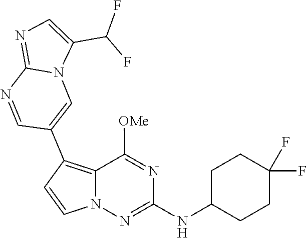 [SMILES: COc1nc(NC2CCC(F)(F)CC2)nn2ccc(-c3cnc4ncc(C(F)F)n4c3)c12] |
| 1645 | 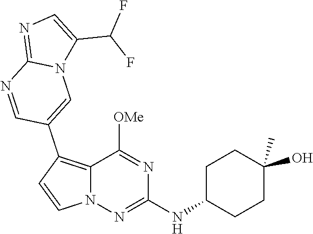 [SMILES: COc1nc(N[C@H]2CC[C@](C)(O)CC2)nn2ccc(-c3cnc4ncc(C(F)F)n4c3)c12] |
| 1646 | 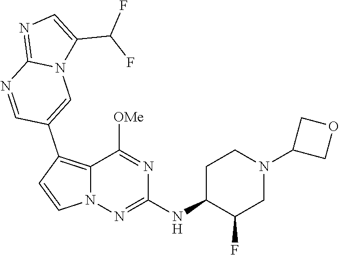 [SMILES: COc1nc(N[C@H]2CCN(C3COC3)C[C@H]2F)nn2ccc(-c3cnc4ncc(C(F)F)n4c3)c12] |
| 1647 | 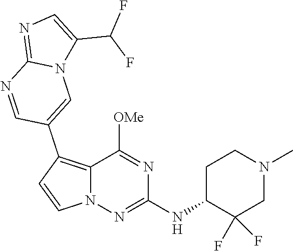 [SMILES: COc1nc(N[C@@H]2CCN(C)CC2(F)F)nn2ccc(-c3cnc4ncc(C(F)F)n4c3)c12] |
| 1648 | 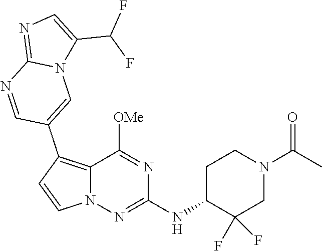 [SMILES: COc1nc(N[C@@H]2CCN(C(C)=O)CC2(F)F)nn2ccc(-c3cnc4ncc(C(F)F)n4c3)c12] |
| 1649 | 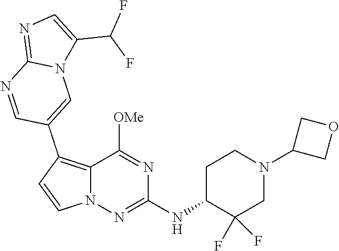 [SMILES: COc1nc(N[C@@H]2CCN(C3COC3)CC2(F)F)nn2ccc(-c3cnc4ncc(C(F)F)n4c3)c12] |
| 1650 | 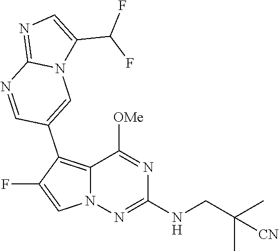 [SMILES: COc1nc(NCC(C)(C)C#N)nn2cc(F)c(-c3cnc4ncc(C(F)F)n4c3)c12] |
| 1651 | 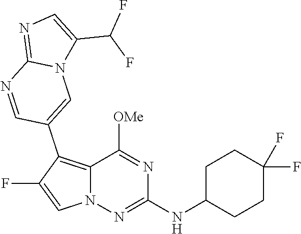 [SMILES: COc1nc(NC2CCC(F)(F)CC2)nn2cc(F)c(-c3cnc4ncc(C(F)F)n4c3)c12] |
| 1652 | 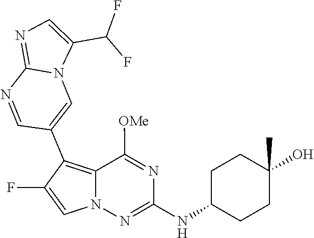 [SMILES: COc1nc(N[C@H]2CC[C@@](C)(O)CC2)nn2cc(F)c(-c3cnc4ncc(C(F)F)n4c3)c12] |
| 1653 | 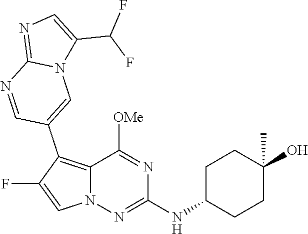 [SMILES: COc1nc(N[C@H]2CC[C@](C)(O)CC2)nn2cc(F)c(-c3cnc4ncc(C(F)F)n4c3)c12] |
| 1654 | 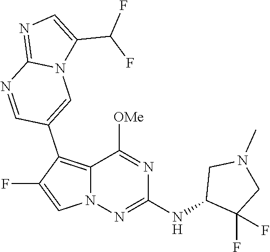 [SMILES: COc1nc(N[C@@H]2CN(C)CC2(F)F)nn2cc(F)c(-c3cnc4ncc(C(F)F)n4c3)c12] |
| 1655 | 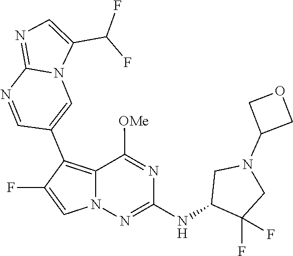 [SMILES: COc1nc(N[C@@H]2CN(C3COC3)CC2(F)F)nn2cc(F)c(-c3cnc4ncc(C(F)F)n4c3)c12] |
| 1656 | 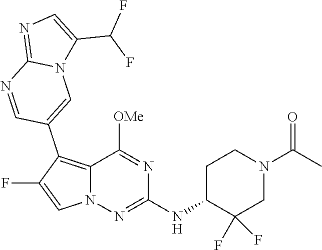 [SMILES: COc1nc(N[C@@H]2CCN(C(C)=O)CC2(F)F)nn2cc(F)c(-c3cnc4ncc(C(F)F)n4c3)c12] |
| 1657 | 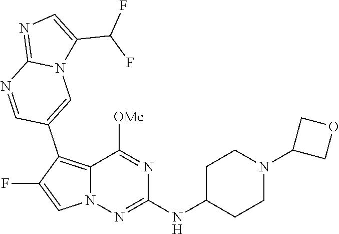 [SMILES: COc1nc(NC2CCN(C3COC3)CC2)nn2cc(F)c(-c3cnc4ncc(C(F)F)n4c3)c12] |
| 1658 | 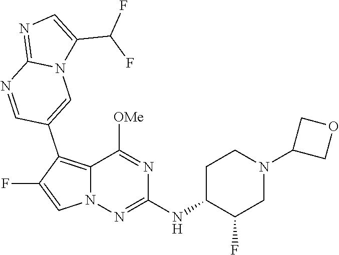 [SMILES: COc1nc(N[C@@H]2CCN(C3COC3)C[C@@H]2F)nn2cc(F)c(-c3cnc4ncc(C(F)F)n4c3)c12] |
| 1659 | 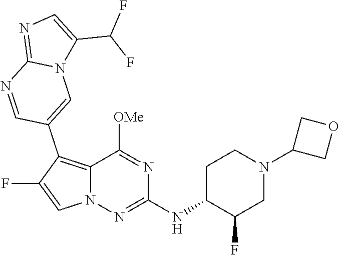 [SMILES: COc1nc(N[C@@H]2CCN(C3COC3)C[C@H]2F)nn2cc(F)c(-c3cnc4ncc(C(F)F)n4c3)c12] |
| 1660 | 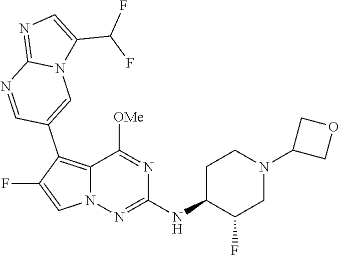 [SMILES: COc1nc(N[C@H]2CCN(C3COC3)C[C@@H]2F)nn2cc(F)c(-c3cnc4ncc(C(F)F)n4c3)c12] |
| 1661 | 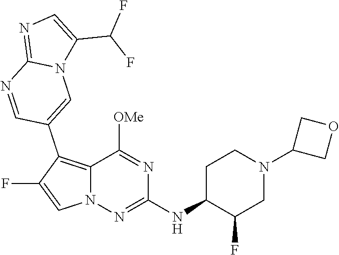 [SMILES: COc1nc(N[C@H]2CCN(C3COC3)C[C@H]2F)nn2cc(F)c(-c3cnc4ncc(C(F)F)n4c3)c12] |
| 1662 | 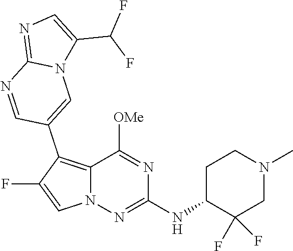 [SMILES: COc1nc(N[C@@H]2CCN(C)CC2(F)F)nn2cc(F)c(-c3cnc4ncc(C(F)F)n4c3)c12] |
| 1663 | 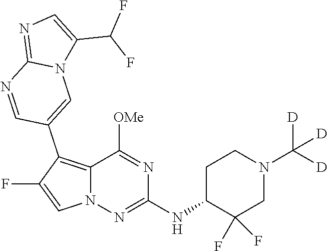 [SMILES: [2H]C([2H])([2H])N1CC[C@@H](Nc2nc(OC)c3c(-c4cnc5ncc(C(F)F)n5c4)c(F)cn3n2)C(F)(F)C1] |
| 1664 | 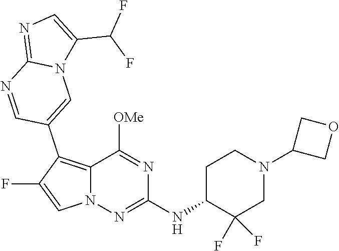 [SMILES: COc1nc(N[C@@H]2CCN(C3COC3)CC2(F)F)nn2cc(F)c(-c3cnc4ncc(C(F)F)n4c3)c12] |
| 1665 | 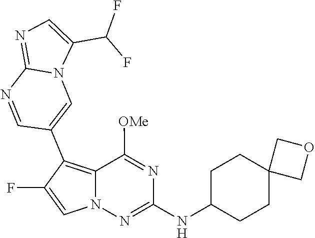 [SMILES: COc1nc(NC2CCC3(CC2)COC3)nn2cc(F)c(-c3cnc4ncc(C(F)F)n4c3)c12] |
| 1666 | 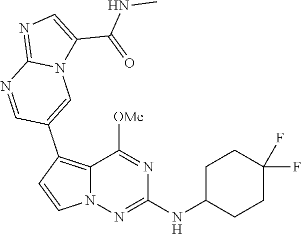 [SMILES: CNC(=O)c1cnc2ncc(-c3ccn4nc(NC5CCC(F)(F)CC5)nc(OC)c34)cn12] |
| 1667 | 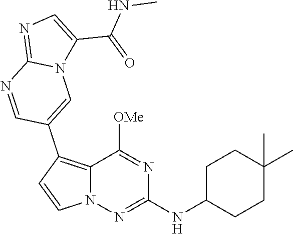 [SMILES: CNC(=O)c1cnc2ncc(-c3ccn4nc(NC5CCC(C)(C)CC5)nc(OC)c34)cn12] |
| 1668 | 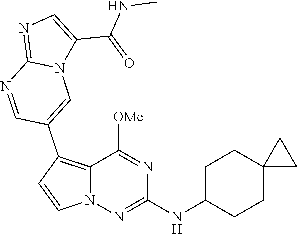 [SMILES: CNC(=O)c1cnc2ncc(-c3ccn4nc(NC5CCC6(CC5)CC6)nc(OC)c34)cn12] |
| 1669 | 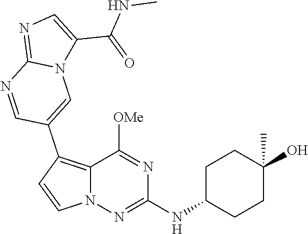 [SMILES: CNC(=O)c1cnc2ncc(-c3ccn4nc(N[C@H]5CC[C@](C)(O)CC5)nc(OC)c34)cn12] |
| 1670 | 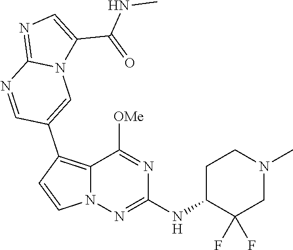 [SMILES: CNC(=O)c1cnc2ncc(-c3ccn4nc(N[C@@H]5CCN(C)CC5(F)F)nc(OC)c34)cn12] |
| 1671 | 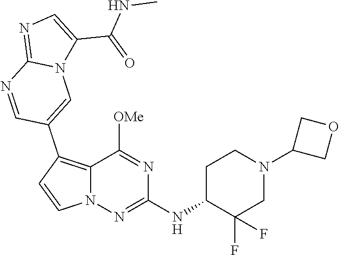 [SMILES: CNC(=O)c1cnc2ncc(-c3ccn4nc(N[C@@H]5CCN(C6COC6)CC5(F)F)nc(OC)c34)cn12] |
| 1672 | 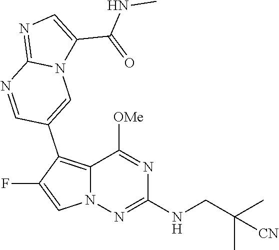 [SMILES: CNC(=O)c1cnc2ncc(-c3c(F)cn4nc(NCC(C)(C)C#N)nc(OC)c34)cn12] |
| 1673 | 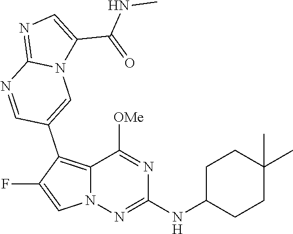 [SMILES: CNC(=O)c1cnc2ncc(-c3c(F)cn4nc(NC5CCC(C)(C)CC5)nc(OC)c34)cn12] |
| 1674 | 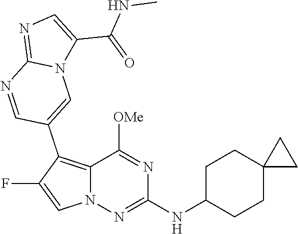 [SMILES: CNC(=O)c1cnc2ncc(-c3c(F)cn4nc(NC5CCC6(CC5)CC6)nc(OC)c34)cn12] |
| 1675 | 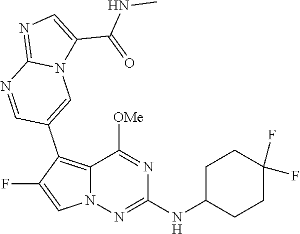 [SMILES: CNC(=O)c1cnc2ncc(-c3c(F)cn4nc(NC5CCC(F)(F)CC5)nc(OC)c34)cn12] |
| 1676 | 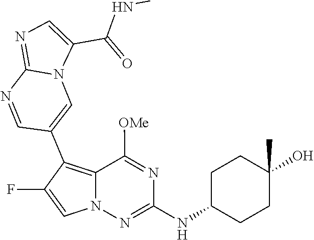 [SMILES: CNC(=O)c1cnc2ncc(-c3c(F)cn4nc(N[C@H]5CC[C@@](C)(O)CC5)nc(OC)c34)cn12] |
| 1677 | 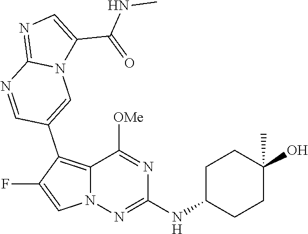 [SMILES: CNC(=O)c1cnc2ncc(-c3c(F)cn4nc(N[C@H]5CC[C@](C)(O)CC5)nc(OC)c34)cn12] |
| 1678 | 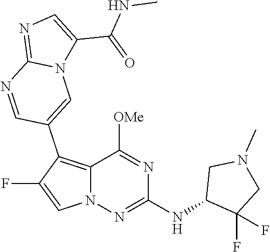 [SMILES: CNC(=O)c1cnc2ncc(-c3c(F)cn4nc(N[C@@H]5CN(C)CC5(F)F)nc(OC)c34)cn12] |
| 1679 | 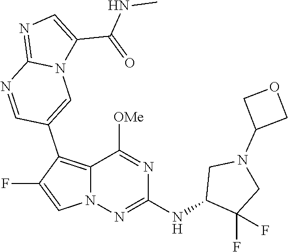 [SMILES: CNC(=O)c1cnc2ncc(-c3c(F)cn4nc(N[C@@H]5CN(C6COC6)CC5(F)F)nc(OC)c34)cn12] |
| 1680 | 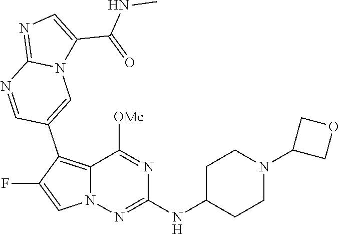 [SMILES: CNC(=O)c1cnc2ncc(-c3c(F)cn4nc(NC5CCN(C6COC6)CC5)nc(OC)c34)cn12] |
| 1681 | 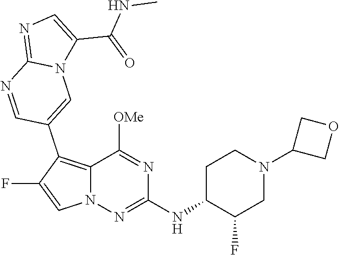 [SMILES: CNC(=O)c1cnc2ncc(-c3c(F)cn4nc(N[C@@H]5CCN(C6COC6)C[C@@H]5F)nc(OC)c34)cn12] |
| 1682 | 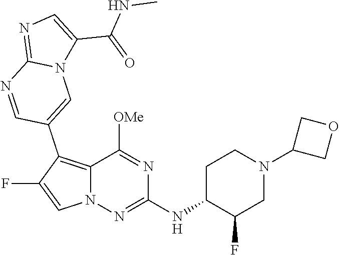 [SMILES: CNC(=O)c1cnc2ncc(-c3c(F)cn4nc(N[C@@H]5CCN(C6COC6)C[C@H]5F)nc(OC)c34)cn12] |
| 1683 | 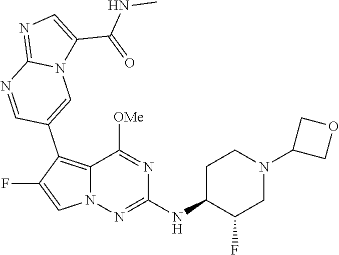 [SMILES: CNC(=O)c1cnc2ncc(-c3c(F)cn4nc(N[C@H]5CCN(C6COC6)C[C@@H]5F)nc(OC)c34)cn12] |
| 1684 | 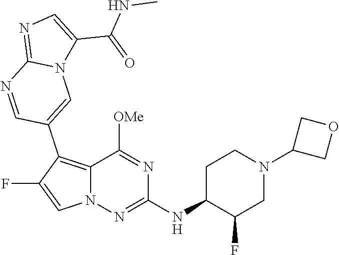 [SMILES: CNC(=O)c1cnc2ncc(-c3c(F)cn4nc(N[C@H]5CCN(C6COC6)C[C@H]5F)nc(OC)c34)cn12] |
| 1685 | 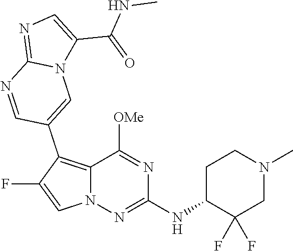 [SMILES: CNC(=O)c1cnc2ncc(-c3c(F)cn4nc(N[C@@H]5CCN(C)CC5(F)F)nc(OC)c34)cn12] |
| 1686 | 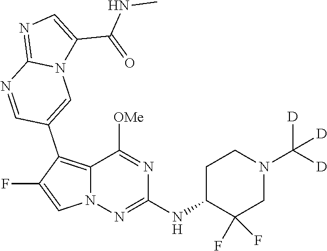 [SMILES: [2H]C([2H])([2H])N1CC[C@@H](Nc2nc(OC)c3c(-c4cnc5ncc(C(=O)NC)n5c4)c(F)cn3n2)C(F)(F)C1] |
| 1687 | 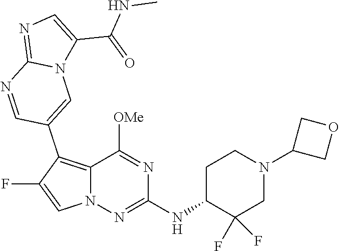 [SMILES: CNC(=O)c1cnc2ncc(-c3c(F)cn4nc(N[C@@H]5CCN(C6COC6)CC5(F)F)nc(OC)c34)cn12] |
| 1688 | 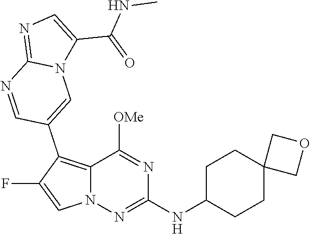 [SMILES: CNC(=O)c1cnc2ncc(-c3c(F)cn4nc(NC5CCC6(CC5)COC6)nc(OC)c34)cn12] |
| 1689 | 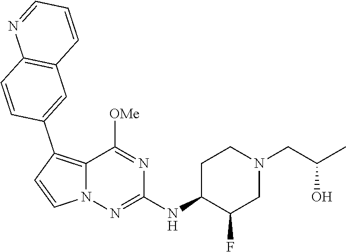 [SMILES: COc1nc(N[C@H]2CCN(C[C@H](C)O)C[C@H]2F)nn2ccc(-c3ccc4ncccc4c3)c12] |
| 1690 | 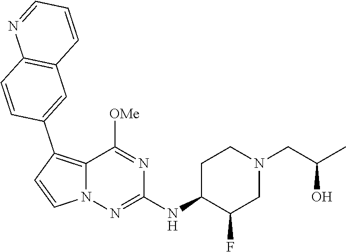 [SMILES: COc1nc(N[C@H]2CCN(C[C@@H](C)O)C[C@H]2F)nn2ccc(-c3ccc4ncccc4c3)c12] |
| 1691 | 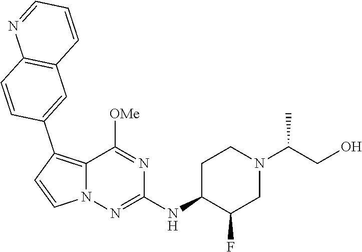 [SMILES: COc1nc(N[C@H]2CCN([C@H](C)CO)C[C@H]2F)nn2ccc(-c3ccc4ncccc4c3)c12] |
| 1692 | 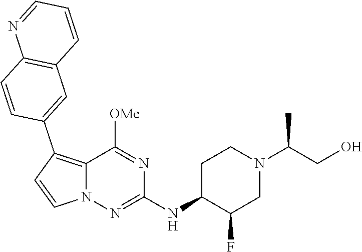 [SMILES: COc1nc(N[C@H]2CCN([C@@H](C)CO)C[C@H]2F)nn2ccc(-c3ccc4ncccc4c3)c12] |
| 1693 | 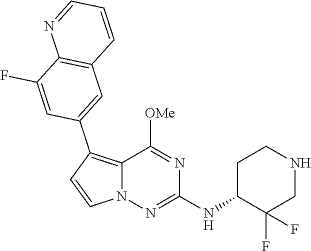 [SMILES: COc1nc(N[C@@H]2CCNCC2(F)F)nn2ccc(-c3cc(F)c4ncccc4c3)c12] |
| 1694 | 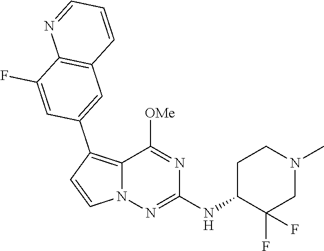 [SMILES: COc1nc(N[C@@H]2CCN(C)CC2(F)F)nn2ccc(-c3cc(F)c4ncccc4c3)c12] |
| 1695 | 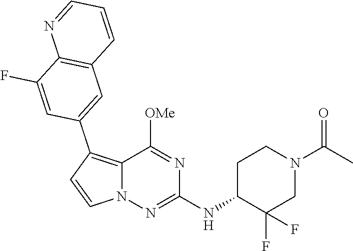 [SMILES: COc1nc(N[C@@H]2CCN(C(C)=O)CC2(F)F)nn2ccc(-c3cc(F)c4ncccc4c3)c12] |
| 1696 | 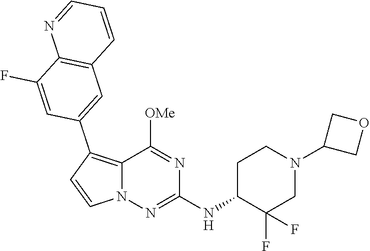 [SMILES: COc1nc(N[C@@H]2CCN(C3COC3)CC2(F)F)nn2ccc(-c3cc(F)c4ncccc4c3)c12] |
| 1697 | 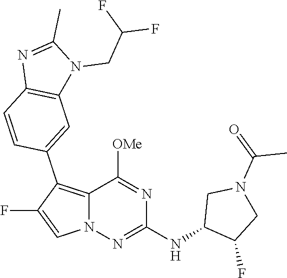 [SMILES: COc1nc(N[C@@H]2CN(C(C)=O)C[C@@H]2F)nn2cc(F)c(-c3ccc4nc(C)n(CC(F)F)c4c3)c12] |
| 1698 | 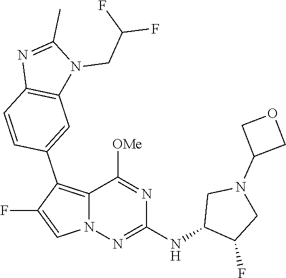 [SMILES: COc1nc(N[C@@H]2CN(C3COC3)C[C@@H]2F)nn2cc(F)c(-c3ccc4nc(C)n(CC(F)F)c4c3)c12] |

### Administration and Pharmaceutical Compositions

Some embodiments include pharmaceutical compositions comprising: (a) a therapeutically effective amount of a compound provided herein, or its corresponding enantiomer, diastereoisomer or tautomer, or pharmaceutically acceptable salt; and (b) a pharmaceutically acceptable carrier.

The compounds provided herein may also be useful in combination (administered together or sequentially) with other known agents.

Non-limiting examples of diseases which can be treated with a combination of a compound of Formula I and another active agent are colorectal cancer, ovarian cancer, hepatocellular carcinoma, head and neck squamous cell carcinoma, acute lymphoblastic leukemia (ALL), pancreatic cancer, brain tumors, acute megakaryoblastic leukemia (AMKL), and osteoarthritis. For example, a compound of Formula I can be combined with one or more chemotherapeutic compounds.

In some embodiments, hepatocellular carcinoma can be treated with a combination of a compound of Formula I and one or more of the following drugs/therapies: sorafenib (Nexavar®); regorafenib (Stivarga®, Regonix®), nivolumab (Opdivo®); lenvatinib (Lenvima®); pembrolizumab (Keytruda®); cabozantinib (Cometriq®, Cabometyx®); 5-fluorouracil (5-FU®); ramucirumab (Cyramza®); combination of gemcitabine and oxaliplatin (GEMOX). Other therapies that can be performed in combination with a compound of Formula I are i) transcatheter arterial chemoembolization (TACE) in combination with doxorubicin (DOXIL®), cisplatin, or mitomycin C (Mitosol®, Mutamycin®, Jelmyto®); ii) low-dose brachytherapy.

In some embodiments, head and neck squamous cell carcinoma can be treated with a combination of a compound of Formula I and one or more of the following drugs/therapies: TransOral Robotic Surgery (TORS); TORS with radiation therapy; larotrectinib (Vitrakvi®); EGFR inhibitors, e.g., erlotinib (Tarceva®), osimertinib (Tagrisso®), neratinib (Nerlynx®), gefitinib (Iressa®), cetuximab (Erbitux®), panitumumab (Vectibix®), dacomitinib (Vizimpro®), lapatinib (Tykerb®), necitumumab (Portrazza), and vandetanib (Caprelsa®).

In some embodiments, acute lymphoblastic leukemia (ALL) can be treated with a combination of a compound of Formula I and one or more of the following drugs/therapies: remission induction therapy; consolidation therapy; nelarabine (Arranon®); asparaginase Erwinia chrysanthemi (Erwinaze®); asparaginase Erwinia chrysanthemi (recombinant)-rywn (Rylaze®); calaspargase Pegol-mknl (Asparlas®); inotuzumab ozogamicin (Besponsa®); blinatumomab (Blincyto®); daunorubicin hydrochloride (Cerubidine®); clofarabine (Clolar®); cyclophosphamide; methotrexate sodium (Trexall®); cytarabine (Cytosar-U®); dasatinib (Sprycel®); dexamethasone; imatinib mesylate (Gleevec®); ponatinib hydrochloride (Iclusig®); mercaptopurine (Purinethol®, Purixan®); tisagenlecleucel (Kymriah®); vincristine sulfate liposome (Marqibo®); pegaspargase (Oncaspar®); prednisone; daunorubicin hydrochloride (Rubidomycin®); and vincristine sulfate.

In some embodiments, pancreatic cancer can be treated with a combination of a compound of Formula I and one or more of the following drugs/therapies: ablation and embolization treatment; gemcitabine (Gemzar®); 5-fluorouracil (5-FU®); oxaliplatin (Eloxatin®); albumin-bound paclitaxel (Abraxane®); capecitabine (Xeloda®); cisplatin; irinotecan (Camptosar®); liposomal irinotecan (Onivyde®); paclitaxel (Taxol®), and docetaxel (Taxotere®).

In some embodiments, brain tumors can be treated with a combination of a compound of Formula I and one or more of the following drugs/therapies: carmustine can be administered by way of a gliadel wafer; for glioblastoma and high-grade glioma, radiation therapy with daily low-dose temozolomide (Temodar®) followed by monthly doses of temozolomide after radiation therapy for 6 months to 1 year; lomustine (Gleostine®), procarbazine (Matulane®), and vincristine (Vincasar®), have been used along with radiation therapy; anti-angiogenesis therapy with bevacizumab (Avastin®, Mvasi®); and targeted therapy using larotrectinib (Vitrakvi®).

In some embodiments, acute megakaryoblastic leukemia (AMKL) can be treated with a combination of a compound of Formula I and one or more of the following drugs/therapies: cytarabine (Cytosar-U®), etoposide (Vepesid®), and anthracycline drugs. Anthracyclines include daunorubicin (Cerubidine®), idarubicin (Idamycin®), and mitoxantrone (Novantrone®).

In some embodiments, acute myeloid leukemia (AML) can be treated with a combination of a compound of Formula I and one or more of the following drugs/therapies: venetoclax and hypomethylating agents (e.g., decitabine, azacitidine), induction chemotherapy (cytarabine and an anthracycline (e.g., daunorubicin or idarubicin), all-trans-retinoic acid (ATRA) and either arsenic trioxide (ATO) monotherapy or an anthracycline), consolidation therapy (cytarabine).

In some embodiments, myelodysplastic syndrome (MDS) can be treated with a combination of a compound of Formula I and one or more of the following drugs/therapies: 5-azacytidine, decitabine, lenalidomide, and decitabine/cedazuridine (Ingovi®).

In some embodiments, colorectal cancer can be treated with a combination of a compound of Formula I and one or more of the following drugs: 5-fluorouracil (5-FU), which can be administered with the vitamin-like drug leucovorin (also called folinic acid); capecitabine (XELODA®), irinotecan (CAMPOSTAR®), oxaliplatin (ELOXATIN®). Examples of combinations of these drugs which could be further combined with a compound of Formula I are FOLFOX (5-FU, leucovorin, and oxaliplatin), FOLFIRI (5-FU, leucovorin, and irinotecan), FOLFOXIRI (leucovorin, 5-FU, oxaliplatin, and irinotecan) and CapeOx (Capecitabine and oxaliplatin). For rectal cancer, chemo with 5-FU or capecitabine combined with radiation may be given before surgery (neoadjuvant treatment).

In some embodiments, ovarian cancer can be treated with a combination of a compound of Formula I and one or more of the following drugs: topotecan, liposomal doxorubicin (DOXIL®), gemcitabine (GEMZAR®), cyclophosphamide (CYTOXAN®), vinorelbine (NAVELBINE®), ifosfamide (IFEX®), etoposide (VP-16), altretamine (HEXALEN®), capecitabine (XELODA®), irinotecan (CPT-11, CAMPTOSAR®), melphalan, pemetrexed (ALIMTA®) and albumin bound paclitaxel (nab-paclitaxel, ABRAXANE®). Examples of combinations of these drugs which could be further combined with a compound of Formula I are TIP (paclitaxel [Taxol], ifosfamide, and cisplatin), VeIP (vinblastine, ifosfamide, and cisplatin) and VIP (etoposide [VP-16], ifosfamide, and cisplatin). Ovarian cancer can also be treated with a combination of a compound of Formula I and immune checkpoint blockade (ICB) therapy.

In some embodiments, a compound of Formula I can be used to treat cancer in combination with any of the following methods: (a) hormone therapy such as aromatase inhibitors, LHRH [luteinizing hormone-releasing hormone] analogs and inhibitors, and others; (b) ablation or embolization procedures such as radiofrequency ablation (RFA), ethanol (alcohol) ablation, microwave thermotherapy and cryosurgery (cryotherapy); (c) chemotherapy using alkylating agents such as cisplatin and carboplatin, oxaliplatin, mechlorethamine, cyclophosphamide, chlorambucil and ifosfamide; (d) chemotherapy using anti-metabolites such as azathioprine and mercaptopurine; (e) chemotherapy using plant alkaloids and terpenoids such as vinca alkaloids (i.e., vincristine, vinblastine, vinorelbine and vindesine) and taxanes; (f) chemotherapy using podophyllotoxin, etoposide, teniposide and docetaxel; (g) chemotherapy using topoisomerase inhibitors such as irinotecan, topotecan, amsacrine, etoposide, etoposide phosphate, and teniposide; (h) chemotherapy using cytotoxic antibiotics such as actinomycin, anthracyclines, doxorubicin, daunorubicin, valrubicin, idarubicin, epirubicin, bleomycin, plicamycin and mitomycin; (i) chemotherapy using tyrosine-kinase inhibitors such as imatinib mesylate (GLEEVEC®, also known as STI-571), gefitinib (Iressa, also known as ZD1839), erlotinib (marketed as TARCEVA®), bortezomib (VELCADE®), tamoxifen, tofacitinib, crizotinib, Bcl-2 inhibitors (e.g., obatoclax, navitoclax (ABT-263), oblimersen (G3139), venetoclax (ABT-199), Gossypol), PARP inhibitors (e.g., iniparib, olaparib, rucaparib, niraparib, talazoparib), PI3K inhibitors (e.g., perifosine in a phase III trial), VEGF receptor 2 inhibitors (e.g., apatinib), AN-152, (AEZS-108), BRAF inhibitors (e.g., vemurafenib, dabrafenib and LGX818), MEK inhibitors (e.g., trametinib and MEK162), CDK inhibitors (e.g., PD-0332991), salinomycin and sorafenib; (j) chemotherapy using monoclonal antibodies such as rituximab (marketed as MABTHERA® or RITUXAN®), trastuzumab (Herceptin also known as ErbB2), cetuximab (marketed as ERBITUX®), and bevacizumab (marketed as AVASTIN®); (k) chemotherapy using KRAS G12C inhibitors such as sotorasib (Lumakras® and Lumykras®), adagrasib (MRTX849), and ARS-3248 (Wellspring Biosciences); (1) chemotherapy using checkpoint inhibitor therapy such as ipilimumab (Yervoy®), nivolumab (Opdivo®), pembrolizumab (Keytruda®), atezolizumab (Tecentriq®), avelumab (Bavencio), durvalumab (Imfinzi), cemiplimab (Libtayo®), and spartalizumab (PDR001); (m) chemotherapy using antibody-drug conjugates (ADC) such as gemtuzumab ozogamicin, brentuximab vedotin, trastuzumab emtansine, inotuzumab ozogamicin, polatuzumab vedotin, enfortumab vedotin, trastuzumab deruxtecan, sacituzumab govitecan, belantamab mafodotin, moxetumomab pasudotox, and loncastuximab tesirine; (n) chemotherapy using proteasome inhibitors such as carfilzomib, lactacystin, disulfiram, salinosporamide A (marizomib), oprozomib, delanzomib, epoxomicin, MG132, β-hydroxy β-methylbutyric acid (HMB), bortezomib, ixazomib (alone or in in combination with lenalidomide and dexamethasone); and (o) radiation therapy.

In some embodiments, a compound of Formula I, can be used to treat diabetes mellitus in combination with any of the following methods: (a) injections of insulin; (b) biguanides such as metformin (Glucophage), phenformin (DBI), and buformin; (c) thiazolidinediones (TZDs) such as rosiglitazone (Avandia), pioglitazone (Actos), and yroglitazone (Rezulin); (d) lyn kinase activators such as glimepiride (Amaryl®) and tolimidone (MLR-1023); (e) secretagogues such as sulfonylureas (non-limiting examples are acetohexamide, carbutamide, chlorpropamide, glycyclamide (tolcyclamide), metahexamide, tolazamide, tolbutamide, glibenclamide (glyburide), glibornuride, gliclazide, glipizide, gliquidone, glisoxepide, glyclopyramide, and glimepiride) and meglitinides (nonlimiting examples are repaglinide (Prandin), nateglinide (Starlix), and mitiglinide (Glufast)); (f) alpha-glucosidase inhibitors such as acarbose (Glucobay, Precose, Prandase), miglitol (Glyset), and voglibose; (g) injectable incretin mimetics such as glucagon-like peptide-1 (GLP-1) and gastric inhibitory peptide (glucose-dependent insulinotropic peptide, GIP), nonlimiting examples of injectable glucagon-like peptide (GLP) analogs and agonists are exenatide (Exendin-4, marketed as Byetta), liraglutide (Victoza, Saxenda), taspoglutide, lixisenatide (Lyxumia), Semaglutide (Ozempic, Rybelsus), dulaglutide (Trulicity), albiglutide (Tanzeum), nonlimiting examples of dipeptidyl peptidase-4 (DPP-4) inhibitors are sitagliptin (Januvia), vildagliptin (Galvus), saxagliptin (Onglyza), linagliptin (Tradjenta), gemigliptin (Zemiglo), anagliptin (Suiny), teneligliptin (Tenelia), alogliptin (Nesina, Vipidia, Kazano, Vipidomet (with metformin), Oseni, Incresync (with pioglitazone)), trelagliptin (Zafatek, Wedica), omarigliptin (MK-3102), evogliptin (Suganon, Evodine), gosogliptin (Saterex), and dutogliptin; (h) injectable amylin analogues such as pramlintide (Symlin); (i) glycosurics (SGLT2 inhibitors) such as canagliflozin (Invokana, Sulisent, Prominad), dapagliflozin (Forxiga, Farxiga, Edistride), empagliflozin (Jardiance, Sciampa-M), ertugliflozin (Steglatro), ipragliflozin (Suglat), luseogliflozin (Lusefi), remogliflozin etabonate (pro-drug of remogliflozin), sergliflozin etabonate (GW869682X), sotagliflozin (Zynquista), and tofogliflozin (CSG452).

In some embodiments, a compound of Formula I can be used to treat osteoarthritis in combination with any of the following methods: (d) injections of a Wnt signaling pathway inhibitor (e.g., lorecivivint); (a) nonsteroidal anti-inflammatory drugs (NSAIDs) such as ibuprofen, naproxen, aspirin and acetaminophen; (b) physical therapy; (c) injections of corticosteroid medications; (d) injections of hyaluronic acid derivatives (e.g., Hyalgan, Synvisc); (e) narcotics, like codeine; (f) in combination with braces and/or shoe inserts or any device that can immobilize or support your joint to help you keep pressure off it (e.g., splints, braces, shoe inserts or other medical devices); (g) realigning bones (osteotomy); (h) joint replacement (arthroplasty); and (i) in combination with a chronic pain class.

In some embodiments, a compound of Formula I can be used to treat Alzheimer's disease in combination with aducanumab (Aduhelm™); acetylcholinesterase inhibitors, e.g., tacrine, rivastigmine (Exelon®), galantamine (Razadyne® and GalantaMind™), and donepezil (Aricept®); and memantine (Axura®, Ebixa®, Namenda®).

Administration of the compounds disclosed herein or the pharmaceutically acceptable salts thereof can be via any of the accepted modes of administration, including, but not limited to, orally, subcutaneously, intravenously, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic devices. In some embodiments, the administration method includes oral or parenteral administration.

Compounds provided herein intended for pharmaceutical use may be administered as crystalline or amorphous products. Pharmaceutically acceptable compositions may include solid, semi-solid, liquid, solutions, colloidal, liposomes, emulsions, suspensions, complexes, coacervates and aerosols. Dosage forms, such as, e.g., tablets, capsules, powders, liquids, suspensions, suppositories, aerosols, implants, controlled release, or the like. They may be obtained, for example, as solid plugs, powders, or films by methods such as precipitation, crystallization, milling, grinding, supercritical fluid processing, coacervation, complex coacervation, encapsulation, emulsification, complexation, freeze drying, spray drying, or evaporative drying. Microwave or radio frequency drying may be used for this purpose. The compounds can also be administered in sustained or controlled release dosage forms, including depot injections, osmotic pumps, pills (tablets and or capsules), transdermal (including electrotransport) patches, implants, and the like, for prolonged and/or timed, pulsed administration at a predetermined rate.

The compounds can be administered either alone or in combination with a conventional pharmaceutical carrier, excipient, or the like. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic carrier may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In one embodiment, the compositions will take the form of a unit dosage form such as a pill or tablet and thus the composition may contain, along with a compound provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives, or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Liquid pharmaceutically administrable compositions can, for example, be prepared by dissolving, dispersing, etc. a compound provided herein and optional pharmaceutical adjuvants in a carrier (e.g., water, saline, aqueous dextrose, glycerol, glycols, ethanol, or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrin derivatives, sorbitan monolaurate, triethanolamine acetate, triethanolamine oleate, and the like).

In some embodiments, the unit dosage of compounds of Formula I is about 0.25 mg/Kg to about 50 mg/Kg in humans.

In some embodiments, the unit dosage of compounds of Formula I is about 0.25 mg/Kg to about 20 mg/Kg in humans.

In some embodiments, the unit dosage of compounds of Formula I is about 0.50 mg/Kg to about 19 mg/Kg in humans.

In some embodiments, the unit dosage of compounds of Formula I is about 0.75 mg/Kg to about 18 mg/Kg in humans.

In some embodiments, the unit dosage of compounds of Formula I is about 1.0 mg/Kg to about 17 mg/Kg in humans.

In some embodiments, the unit dosage of compounds of Formula I is about 1.25 mg/Kg to about 16 mg/Kg in humans.

In some embodiments, the unit dosage of compounds of Formula I is about 1.50 mg/Kg to about 15 mg/Kg in humans.

In some embodiments, the unit dosage of compounds of Formula I is about 1.75 mg/Kg to about 14 mg/Kg in humans.

In some embodiments, the unit dosage of compounds of Formula I is about 2.0 mg/Kg to about 13 mg/Kg in humans.

In some embodiments, the unit dosage of compounds of Formula I is about 3.0 mg/Kg to about 12 mg/Kg in humans.

In some embodiments, the unit dosage of compounds of Formula I is about 4.0 mg/Kg to about 11 mg/Kg in humans.

In some embodiments, the unit dosage of compounds of Formula I is about 5.0 mg/Kg to about 10 mg/Kg in humans.

In some embodiments, the compositions are provided in unit dosage forms suitable for single administration.

In some embodiments, the compositions are provided in unit dosage forms suitable for twice a day administration.

In some embodiments, the compositions are provided in unit dosage forms suitable for three times a day administration.

Injectables can be prepared in conventional forms, either as liquid solutions, colloid, liposomes, complexes, coacervate or suspensions, as emulsions, or in solid forms suitable for reconstitution in liquid prior to injection. The percentage of a compound provided herein contained in such parenteral compositions is highly dependent on the specific nature thereof, as well as the activity of the compound and the needs of the patient. However, percentages of active ingredient of 0.01% to 10% in solution are employable and could be higher if the composition is a solid or suspension, which could be subsequently diluted to the above percentages.

In some embodiments, the composition comprises about 0.1-10% ofthe active agent in solution.

In some embodiments, the composition comprises about 0.1-5% of the active agent in solution.

In some embodiments, the composition comprises about 0.1-4% of the active agent in solution.

In some embodiments, the composition comprises about 0.15-3% of the active agent in solution.

In some embodiments, the composition comprises about 0.2-2% of the active agent in solution.

In some embodiments, the compositions are provided in dosage forms suitable for continuous dosage by intravenous infusion over a period of about 1-96 hours.

In some embodiments, the compositions are provided in dosage forms suitable for continuous dosage by intravenous infusion over a period of about 1-72 hours.

In some embodiments, the compositions are provided in dosage forms suitable for continuous dosage by intravenous infusion over a period of about 1-48 hours.

In some embodiments, the compositions are provided in dosage forms suitable for continuous dosage by intravenous infusion over a period of about 1-24 hours.

In some embodiments, the compositions are provided in dosage forms suitable for continuous dosage by intravenous infusion over a period of about 1-12 hours.

In some embodiments, the compositions are provided in dosage forms suitable for continuous dosage by intravenous infusion over a period of about 1-6 hours.

In some embodiments, these compositions can be administered by intravenous infusion to humans at doses of about 5 mg/m² to about 300 mg/m².

In some embodiments, these compositions can be administered by intravenous infusion to humans at doses of about 5 mg/m² to about 200 mg/m².

In some embodiments, these compositions can be administered by intravenous infusion to humans at doses of about 5 mg/m² to about 100 mg/m².

In some embodiments, these compositions can be administered by intravenous infusion to humans at doses of about 10 mg/m² to about 50 mg/m².

In some embodiments, these compositions can be administered by intravenous infusion to humans at doses of about 50 mg/m² to about 200 mg/m².

In some embodiments, these compositions can be administered by intravenous infusion to humans at doses of about 75 mg/m² to about 175 mg/m².

In some embodiments, these compositions can be administered by intravenous infusion to humans at doses of about 100 mg/m² to about 150 mg/m².

It is to be noted that concentrations and dosage values may also vary depending on the specific compound and the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In one embodiment, the compositions can be administered to the respiratory tract (including nasal and pulmonary) e.g., through a nebulizer, metered-dose inhalers, atomizer, mister, aerosol, dry powder inhaler, insufflator, liquid instillation or other suitable device or technique.

In some embodiments, aerosols intended for delivery to the nasal mucosa are provided for inhalation through the nose. For optimal delivery to the nasal cavities, inhaled particle sizes of about 5 to about 100 microns are useful, with particle sizes of about 10 to about 60 microns being preferred. For nasal delivery, a larger inhaled particle size may be desired to maximize impaction on the nasal mucosa and to minimize or prevent pulmonary deposition of the administered formulation. In some embodiments, aerosols intended for delivery to the lung are provided for inhalation through the nose or the mouth. For delivery to the lung, inhaled aerodynamic particle sizes of about less than 10 μm are useful (e.g., about 1 to about 10 microns). Inhaled particles may be defined as liquid droplets containing dissolved drug, liquid droplets containing suspended drug particles (in cases where the drug is insoluble in the suspending medium), dry particles of pure drug substance, drug substance incorporated with excipients, liposomes, emulsions, colloidal systems, coacervates, aggregates of drug nanoparticles, or dry particles of a diluent which contain embedded drug nanoparticles.

In some embodiments, compounds of Formula I disclosed herein intended for respiratory delivery (either systemic or local) can be administered as aqueous formulations, as non-aqueous solutions, or suspensions, as suspensions or solutions in halogenated hydrocarbon propellants with or without alcohol, as a colloidal system, as emulsions, coacervates, or as dry powders. Aqueous formulations may be aerosolized by liquid nebulizers employing either hydraulic or ultrasonic atomization or by modified micropump systems (like the soft mist inhalers, the Aerodose® or the AERx® systems). Propellant-based systems may use suitable pressurized metered-dose inhalers (pMDIs). Dry powders may use dry powder inhaler devices (DPIs), which are capable of dispersing the drug substance effectively. A desired particle size and distribution may be obtained by choosing an appropriate device.

In some embodiments, the compositions of Formula I disclosed herein can be administered to the ear by various methods. For example, a round window catheter (e.g., U.S. Pat. Nos. 6,440,102 and 6,648,873) can be used.

Alternatively, formulations can be incorporated into a wick for use between the outer and middle ear (e.g., U.S. Pat. No. 6,120,484) or absorbed to collagen sponge or other solid support (e.g., U.S. Pat. No. 4,164,559).

If desired, formulations of the disclosure can be incorporated into a gel formulation (e.g., U.S. Pat. Nos. 4,474,752 and 6,911,211).

In some embodiments, compounds of Formula I disclosed herein intended for delivery to the ear can be administered via an implanted pump and delivery system through a needle directly into the middle or inner ear (cochlea) or through a cochlear implant stylet electrode channel or alternative prepared drug delivery channel such as but not limited to a needle through temporal bone into the cochlea.

Other options include delivery via a pump through a thin film coated onto a multichannel electrode or electrode with a specially imbedded drug delivery channel (pathways) carved into the thin film for this purpose. In other embodiments the acidic or basic solid compound of Formula I can be delivered from the reservoir of an external or internal implanted pumping system.

Formulations of the disclosure also can be administered to the ear by intratympanic injection into the middle ear, inner ear, or cochlea (e.g., U.S. Pat. No. 6,377,849 and Ser. No. 11/337,815).

Intratympanic injection of therapeutic agents is the technique of injecting a therapeutic agent behind the tympanic membrane into the middle and/or inner ear. In one embodiment, the formulations described herein are administered directly onto the round window membrane via transtympanic injection. In another embodiment, the ion channel modulating agent auris-acceptable formulations described herein are administered onto the round window membrane via a non-transtympanic approach to the inner ear. In additional embodiments, the formulation described herein is administered onto the round window membrane via a surgical approach to the round window membrane comprising modification of the crista fenestrae cochleae.

In some embodiments, the compounds of Formula I are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG (like PEG ointments), and the like.

Suppositories for rectal administration ofthe drug (either as a solution, colloid, suspension or a complex) can be prepared by mixing a compound provided herein with a suitable non-irritating excipient that is solid at ordinary temperatures but liquid at the rectal temperature and will therefore melt or erode/dissolve in the rectum and release the compound. Such materials include cocoa butter, glycerinated gelatin, hydrogenated vegetable oils, poloxamers, mixtures of polyethylene glycols of various molecular weights and fatty acid esters of polyethylene glycol. In suppository forms of the compositions, a low-melting wax such as, but not limited to, a mixture of fatty acid glycerides, optionally in combination with cocoa butter, is first melted.

Solid compositions can be provided in various different types of dosage forms, depending on the physicochemical properties of the compound provided herein, the desired dissolution rate, cost considerations, and other criteria. In one of the embodiments, the solid composition is a single unit. This implies that one unit dose of the compound is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In one embodiment, the solid composition is a highly porous lyophilized form. Such lyophilizates, sometimes also called wafers or lyophilized tablets, are particularly useful for their rapid disintegration, which also enables the rapid dissolution of the compound.

On the other hand, for some applications the solid composition may also be formed as a multiple unit dosage form as defined above. Examples of multiple units are powders, granules, microparticles, pellets, mini-tablets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution. Effervescent compositions are also contemplated to aid the quick dispersion and absorption of the compound.

Another type of multiparticulate system which is also capable of achieving rapid drug dissolution is that of powders, granules, or pellets from water-soluble excipients which are coated with a compound provided herein so that the compound is located at the outer surface of the individual particles. In this type of system, the water-soluble low molecular weight excipient may be useful for preparing the cores of such coated particles, which can be subsequently coated with a coating composition comprising the compound and, for example, one or more additional excipients, such as a binder, a pore former, a saccharide, a sugar alcohol, a film-forming polymer, a plasticizer, or other excipients used in pharmaceutical coating compositions.

Also provided herein are kits. Typically, a kit includes one or more compounds or compositions as described herein. In certain embodiments, a kit can include one or more delivery systems, e.g., for delivering or administering a compound as provided herein, and directions for use of the kit (e.g., instructions for treating a patient). In another embodiment, the kit can include a compound or composition as described herein and a label that indicates that the contents are to be administered to a patient with cancer. In another embodiment, the kit can include a compound or composition as described herein and a label that indicates that the contents are to be administered to a patient with one or more of glioblastoma, ovarian, breast, pancreatic cancers, acute lymphoblastic leukemia, acute megakaryoblastic leukemia, chronic myeloid leukemia, Alzheimer's disease, amyotrophic lateral sclerosis, CDKL5 deficiency disorder, Down syndrome, frontotemporal dementia with parkinsonism-17 (FTDP-17), Lewy body dementia, Parkinson's disease, Pick's disease, autism, dementia, epilepsy, Huntington's disease, and multiple sclerosis.

### Methods of Treatment

The compounds and compositions provided herein can be used as inhibitors of DYRK1A, and thus can be used to treat a variety of disorders and diseases in which over expression of DYRK1A is implicated, such as cancer and neurological conditions/disorders/diseases. Non-limiting examples of diseases which can be treated with the compounds and compositions provided herein include a variety of cancers, Alzheimer's disease, amyotrophic lateral sclerosis, CDKL5 deficiency disorder, Down Syndrome, frontotemporal dementia with parkinsonism-17 (FTDP-17), Lewy body dementia, Parkinson's disease, Pick's disease, and additional diseases with pronounced neurodegeneration such as autism, dementia, epilepsy, Huntington's disease, multiple sclerosis; diseases and disorders associated with acquired brain injury such as chronic traumatic encephalopathy, traumatic brain injury, tumor, stroke, tauopathies (e.g., Pick's disease, progressive supranuclear palsy, corticobasal degeneration, argyrophilic grain disease, globular glial tauopathies, primary age-related tauopathy, which includes neurofibrillary tangle dementia, chronic traumatic encephalopathy (CTE), frontotemporal lobar degeneration with tau inclusions (FTLD-tau), and aging-related tau astrogliopathy. Clinical symptoms include frontotemporal dementia, corticobasal syndrome, Richardson syndrome, parkinsonism, pure akinesia with gait freezing and, rarely, motor neuron symptoms or cerebellar ataxia, diabetes, psoriasis, knee osteoarthritis, tendinopathy, human immunodeficiency virus type 1 (HIV-1), human cytomegalovirus (HCMV), hepatitis C virus (HCV), and herpes simplex virus 1 (HSV-1).

The gene encoding DYRK1A is located on chromosome 21, within the Down syndrome critical region (DSCR), the triploidy of which is responsible for most Down syndrome-associated deficiencies (FEBS Journal (2011), 278, 246-256). There is considerable genetical and pharmacological evidence showing that the mere 1.5-fold overexpression of DYRK1A is responsible for most cognitive deficits observed in Down syndrome patients (Pharmacology & Therapeutics (2019), 194, 199-221 and Brain Science (2018), 8(10), 187). Genetical normalization of DYRK1A levels or pharmacological inhibition of its catalytic activity restores cognitive functions. The development of pharmacological inhibitors of DYRK1A is a major avenue for the treatment of cognitive deficits associated with Down syndrome.

DYRK1A and DYRK1B are utilized during human cytomegalovirus (HCMV) placental replication. Inhibition of DYRKs prevent replication of various viruses, including hepatitis C virus (HCV), human cytomegalovirus (HCMV), human immunodeficiency virus type 1 (HIV-1), and herpes simplex virus 1 (HSV-1) (Journal of Virology (2020), 94(6) and PLoS ONE (2015), 10, e0144229).

There is a growing body of evidence showing that DYRK1A/1B inhibitors induce the proliferation of insulin-producing pancreatic β-cells, making DYRK1A/1B kinases attractive therapeutic targets for β-cell regeneration for both type 1 and type 2 diabetes mellitus and gestational diabetes (Nature Communications (2015), 6(8372); Diabetes (2016), 65(6), 1660-1671; JCI Insight (2020), 5(1), e132594; Science Translational Medicine (2020), 12(530); International Journal ofMolecular Sciences (2021), 22(16), 9083; and Journal of Medicinal Chemistry (2021), 64(6), 2901-2922). Other forms of diabetes that may be treated with DYRK inhibitors are maturity onset diabetes of the young (MODY, monogenic diabetes), cases of diabetes that are caused by the body's tissue receptors not responding to insulin, double diabetes (when a type 1 diabetic becomes insulin resistant), diabetes associated with excessive secretion of insulin-antagonistic hormones, malnutrition-related diabetes mellitus (ICD-10 code E12), and diabetes caused by any genetic mutations (autosomal or mitochondrial) that leads to defects in beta cell function.

There is abundant literature linking DYRK1A with solid cancers and leukemias (Pharmacology & Therapeutics (2015), 151, 87-98; Cancers (2020), 12(8), 2106; and Cellular and Molecular Life Sciences (2021), 78, 603-619). The most prominent examples are pancreatic cancer (Gut (2019), 68(8), 1465-1476 and Gene (2020), 758, 144960), brain tumors, glioblastoma (Journal of Clinical Investigation (2013), 123(6), 2475-2487), acute megakaryoblastic leukemia (AMKL) (Journal of Clinical Investigation (2012), 122(3), 948-962), and acute lymphoblastic leukemia (ALL) (Journal of Clinical Investigation (2021), 131(1), e135937). Other cancers linked to DYRK1A are ovarian (Frontiers in Oncology (2021), 11, 637193), head and neck squamous cell carcinoma (Scientific Reports (2016), 6, 36132), hepatocellular carcinoma (Cell Death & Disease (2021), 12, 125), DYRK1A regulates DNA damage response (Scientific Reports (2019), 9, 6014 and Scientific Reports (2019), 9, 6539). In some situations, DYRK1A appears to function as a tumor-suppressor protein (Molecular & Cellular Oncology (2015), 2(1), e970048 and Nature (2016), 529, 172-177).

Other cancers can also be treated with the compounds and compositions described herein.

More particularly, cancers that may be treated by the compounds, compositions and methods described herein include, but are not limited to, the following:

1) Breast cancers, including, for example ER breast cancer, ER⁻ breast cancer, her²⁻ breast cancer, her2⁺ breast cancer, stromal tumors such as fibroadenomas, phyllodes tumors, and sarcomas, and epithelial tumors such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; chemoresistant breast cancers (TNBC), and miscellaneous malignant neoplasms. Further examples of breast cancers can include luminal A, luminal B, basal A, basal B, and triple negative breast cancer, which is estrogen receptor negative (ER⁻), progesterone receptor negative, and her2 negative (her2⁻). In some embodiments, the breast cancer may have a high risk Oncotype score.

2) Cardiac cancers, including, for example sarcoma, e.g., angiosarcoma, fibrosarcoma, rhabdomyosarcoma, and liposarcoma; myxoma; rhabdomyoma; fibroma; lipoma and teratoma.

3) Lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; chemoresistant small cell lung cancer (SCLC), and mesothelioma.

4) Gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; colon cancers with APC gene mutations; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma.

5) Genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma.

6) Liver cancers, including, for example, hepatoma, e.g., hepatocellular carcinoma; cholangiocarcinoma; hepatoblastoma; angiosarcoma; hepatocellular adenoma; and hemangioma.

7) Bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors.

8) Nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, oligodendrocytoma, schwannoma, retinoblastoma, and congenital tumors; and cancers of the spinal cord, e.g., neurofibroma, meningioma, glioma, and sarcoma.

9) Gynecological cancers, including, for example, cancers of the uterus, e.g., endometrial cancers (e.g., carcinoma, endometrioid adenocarcinoma, serous carcinoma, clear cell carcinoma, mucinous carcinomas, mixed or undifferentiated carcinoma (including mixed M0llerian tumor), endometrial stromal sarcoma, squamous cell carcinoma of the endometrium, urothelial carcinoma, endometrial cancer with CTNNB1 mutations); cancers of the cervix, e.g., cervical carcinoma, and pre tumor cervical dysplasia; cancers of the ovaries, e.g., BRCA-mutant ovarian cancer, surface epithelial-stromal tumors (epithelial ovarian cancer (Type 1 (endometroid, mucinous, clear cell, low grade serous) or Type 2 (poorly differentiated, carcinosarcoma, and high grade serous))), ovarian carcinoma, including serous cystadenocarcinoma, mucinous cystadenocarcinoma, endometrioid tumors, small cell ovarian cancer (small cell ovarian cancer of hypercalcemic type, small cell ovarian cancer of pulmonary type) unclassified carcinoma, granulosa theca cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma, primary fallopian tube cancer; Primary peritoneal cancer (also known as serous surface papillary carcinoma, primary peritoneal carcinoma, extra-ovarian serous carcinoma, primary serous papillary carcinoma, and psammomacarcinoma).

10) Hematologic cancers, including, for example, cancers of the blood, e.g., acute myeloid leukemia, chronic myeloid leukemia, myelodysplastic syndromes (refractory cytopenia with unilineage dysplasia (refractory anemia, refractory neutropenia, and refractory thrombocytopenia), refractory anemia with ring sideroblasts, refractory cytopenia with multilineage dysplasia, refractory anemias with excess blasts I and II, refractory cytopenia of childhood), and myeloproliferative neoplasms, acute lymphoblastic leukemia, chronic lymphocytic leukemia, myeloproliferative diseases, multiple myeloma, myelodysplastic syndrome, myelodysplastic-myeloproliferative diseases, Hodgkin's lymphoma, non-Hodgkin's lymphoma (malignant lymphoma) and Waldenstrom's macroglobulinemia.

11) Skin cancers and skin disorders, including, for example, malignant melanoma and metastatic melanoma, basal cell carcinoma, squamous cell carcinoma, Kaposi's sarcoma, moles dysplastic nevi, lipoma, angioma, dermatofibroma, keloids, and scleroderma.

12) Adrenal gland cancers, including, for example, neuroblastoma.

13) Soft-tissue sarcomas (STS) such as fibrosarcoma, malignant fibrous histiocytoma, dermatofibrosarcoma, liposarcoma, rhabdomyosarcoma, leiomyosarcoma, hemangiosarcoma, Kaposi's sarcoma, lymphangiosarcoma, synovial sarcoma, malignant peripheral nerve sheath tumors (also called neurofibrosarcomas, malignant schwannomas, and neurogenic sarcomas), neurofibrosarcoma, extraskeletal chondrosarcoma, extraskeletal osteosarcoma, extraskeletal myxoid chondrosarcoma, extraskeletal mesenchymal, embryonal, alveolar soft part sarcoma, and infantile hemangio-pericytoma.

More particularly, tumors of the central nervous system that may be treated by the compounds, compositions and methods described herein include:

1) Astrocytic tumors, e.g., diffuse astrocytoma (fibrillary, protoplasmic, gemistocytic, mixed), anaplastic (malignant) astrocytoma, glioblastoma multiforme (giant cell glioblastoma and gliosarcoma), pilocytic astrocytoma (pilomyxoid astrocytoma), pleomorphic xanthoastrocytoma, subependymal giant cell astrocytoma, and gliomatosis cerebri.

2) Oligodendroglial tumors, e.g., oligodendroglioma and anaplastic oligodendroglioma.

3) Oligoastrocytic tumors, e.g., oligoastrocytoma and anaplastic oligoastrocytoma.

4) Ependymal tumors, e.g., subependymoma, myxopapillary ependymoma, ependymoma, (cellular, papillary, clear cell, tanycytic), and anaplastic (malignant) ependymoma.

5) Choroid plexus tumors, e.g., choroid plexus papilloma, atypical choroid plexus papilloma, and choroid plexus carcinoma.

6) Neuronal and mixed neuronal-glial tumors, e.g., gangliocytoma, ganglioglioma, dysembryoplastic neuroepithelial tumor (DNET), dysplastic gangliocytoma of the cerebellum (Lhermitte-Duclos), desmoplastic infantile astrocytoma/ganglioglioma, central neurocytoma, anaplastic ganglioglioma, extraventricular neurocytoma, cerebellar liponeurocytoma, Papillary glioneuronal tumor, Rosette-forming glioneuronal tumor of the fourth ventricle, and paraganglioma of the filum terminale.

7) Pineal tumors, e.g., pineocytoma, pineoblastoma, papillary tumors ofthe pineal region, and pineal parenchymal tumor of intermediate differentiation.

8) Embryonal tumors, e.g., medulloblastoma (medulloblastoma with extensive nodularity, anaplastic medulloblastoma, desmoplastic, large cell, melanotic, medullomyoblastoma), medulloepithelioma, supratentorial primitive neuroectodermal tumors, and primitive neuroectodermal tumors (PNETs) such as neuroblastoma, ganglioneuroblastoma, ependymoblastoma, and atypical teratoid/rhabdoid tumor.

9) Neuroblastic tumors, e.g., olfactory (esthesioneuroblastoma), olfactory neuroepithelioma, and neuroblastomas of the adrenal gland and sympathetic nervous system.

10) Glial tumors, e.g., astroblastoma, chordoid glioma of the third ventricle, and angiocentric glioma.

11) Tumors of cranial and paraspinal nerves, e.g., schwannoma, neurofibroma Perineurioma, and malignant peripheral nerve sheath tumor.

12) Tumors of the meninges such as tumors of meningothelial cells, e.g., meningioma (atypical meningioma and anaplastic meningioma); mesenchymal tumors, e.g., lipoma, angiolipoma, hibernoma, liposarcoma, solitary fibrous tumor, fibrosarcoma, malignant fibrous histiocytoma, leiomyoma, leiomyosarcoma, rhabdomyoma, rhabdomyosarcoma, chondroma, chondrosarcoma, osteoma, osteosarcoma, osteochondroma, haemangioma, epithelioid hemangioendothelioma, haemangiopericytoma, anaplastic haemangiopericytoma, angiosarcoma, Kaposi Sarcoma, and Ewing Sarcoma; primary melanocytic lesions, e.g., diffuse melanocytosis, melanocytoma, malignant melanoma, meningeal melanomatosis; and hemangioblastomas.

13) Tumors of the hematopoietic system, e.g., malignant Lymphomas, plasmocytoma, and granulocytic sarcoma.

14) Germ cell tumors, e.g., germinoma, embryonal carcinoma, yolk sac tumor, choriocarcinoma, teratoma, and mixed germ cell tumors.

15) Tumors of the sellar region, e.g., craniopharyngioma, granular cell tumor, pituicytoma, and spindle cell oncocytoma of the adenohypophysis.

Cancers may be solid tumors that may or may not be metastatic. Cancers may also occur, as in leukemia, as a diffuse tissue. Thus, the term “tumor cell,” as provided herein, includes a cell afflicted by any one of the above identified disorders.

A method of treating cancer using a compound or composition as described herein may be combined with existing methods of treating cancers, for example by chemotherapy, irradiation, or surgery (e.g., oophorectomy). In some embodiments, a compound or composition can be administered before, during, or after another anticancer agent or treatment.

There is mounting evidence for a role of DYRK1A in the onset of Alzheimer's Disease (Future Medicinal Chemistry (2016), 8(6), 681-696 and European Journal of Medicinal Chemistry (2018), 158, 559-592). DYRK1A phosphorylates key substrates involved in Alzheimer's Disease and dementia: Tau, septin 4, amyloid precursor protein (APP), presenilin 1, neprilysin, Munc18-1, α-synuclein, RCAN1, and β-tubulin. By modulating alternative splicing of Tau exon 10, DYRK1A favors the production of the 3R-Tau splice isoform (characteristic for DS/AD/tauopathy) over the 4R-Tau isoform (Journal of Biological Chemistry (2015), 290, 15219-15237).

Genome-wide association studies (GWAS) have revealed that DYRK1A is a risk factor for Parkinson's Disease (The Lancet Neurology (2019), 18(12), 1091-1102). DYRK1A phosphorylates key factors for Parkinson's Disease such as parkin, septin 4, and α-synuclein. Upregulation of micro-RNAs specific for Parkinson's Disease targets DYRK1A expression. There is further evidence that DYRK1A expression is increased in Parkinson's Disease and in Pick's disease (Neurobiology of Disease (2005), 20(2), 392-400).

The compounds and compositions provided herein can be used as inhibitors and/or modulators of the enzyme DYRK1A, and thus can be used to treat a variety of disorders and diseases associated with tau protein, including, but not limited to, Alzheimer's disease, amyotrophic lateral sclerosis (ALS), down syndrome, frontotemporal dementia (FTD) including FTD with parkinsonism-17 (FTDP-17), behavioural variant frontotemporal dementia (bvFTD), FTD in patients with motor neuron disease (MND) (typically amyotrophic lateral sclerosis, also called FTD-ALS), corticobasal degeneration (CBD) (also called corticobasal ganglionic degeneration), progressive supranuclear palsy, primary progressive aphasia (PPA), globular glial tauopathy (GGT), myotonic dystrophy type 1 (DM1) (also called Steinert disease), myotonic dystrophy type 2 (DM2) (also called proximal myotonic myopathy), Guam complex, argyrophilic grain disease, dementia pugilistica, post-encephalitic parkinsonism, Lewy body dementia, Parkinson's disease, Pick's disease, and additional diseases with pronounced neurodegeneration such as autism, dementia, epilepsy, Huntington's disease, multiple sclerosis; diseases and disorders associated with acquired brain injury such as chronic traumatic encephalopathy, traumatic brain injury, tumor, and stroke.

Non-limiting examples of neurological disorders (e.g., neurological conditions and neurological diseases) which can be treated with the compounds and compositions provided herein include Alzheimer's disease, aphasia, apraxia, arachnoiditis, ataxia telangiectasia, attention deficit hyperactivity disorder, auditory processing disorder, autism, alcoholism, Bell's palsy, bipolar disorder, brachial plexus injury, Canavan disease, carpal tunnel syndrome, causalgia, central pain syndrome, central pontine myelinolysis, centronuclear myopathy, cephalic disorder, cerebral aneurysm, cerebral arteriosclerosis, cerebral atrophy, cerebral gigantism, cerebral palsy, cerebral vasculitis, cervical spinal stenosis, Charcot-Marie-Tooth disease, Chiari malformation, chronic fatigue syndrome, chronic inflammatory demyelinating polyneuropathy (CIDP), chronic pain, Coffin-Lowry syndrome, complex regional pain syndrome, compression neuropathy, congenital facial diplegia, corticobasal degeneration, cranial arteritis, craniosynostosis, Creutzfeldt-Jakob disease, cumulative trauma disorder, Cushing's syndrome, cytomegalic inclusion body disease (CIBD), Dandy-Walker syndrome, Dawson disease, De Morsier's syndrome, Dejerine-Klumpke palsy, Dejerine-Sottas disease, delayed sleep phase syndrome, dementia, dermatomyositis, developmental dyspraxia, diabetic neuropathy, diffuse sclerosis, Dravet syndrome, dysautonomia, dyscalculia, dysgraphia, dyslexia, dystonia, empty sella syndrome, encephalitis, encephalocele, encephalotrigeminal angiomatosis, encopresis, epilepsy, Erb's palsy, erythromelalgia, essential tremor, Fabry's disease, Fahr's syndrome, familial spastic paralysis, febrile seizure, Fisher syndrome, Friedreich's ataxia, fibromyalgia, Foville's syndrome, Gaucher's disease, Gerstmann's syndrome, giant cell arteritis, giant cell inclusion disease, globoid cell leukodystrophy, gray matter heterotopia, Guillain-Barré syndrome, HTLV-1 associated myelopathy, Hallervorden-Spatz disease, hemifacial spasm, hereditary spastic paraplegia, heredopathia atactica polyneuritiformis, herpes zoster oticus, herpes zoster, Hirayama syndrome, holoprosencephaly, Huntington's disease, hydranencephaly, hydrocephalus, hypercortisolism, hypoxia, immune-mediated encephalomyelitis, inclusion body myositis, incontinentia pigmenti, infantile phytanic acid storage disease, infantile Refsum disease, infantile spasms, inflammatory myopathy, intracranial cyst, intracranial hypertension, Joubert syndrome, Karak syndrome, Kearns-Sayre syndrome, Kennedy disease, Kinsboume syndrome, Klippel Feil syndrome, Krabbe disease, Kugelberg-Welander disease, kuru, Lafora disease, Lambert-Eaton myasthenic syndrome, Landau-Kleffner syndrome, lateral medullary (Wallenberg) syndrome, Leigh's disease, Lennox-Gastaut syndrome, Lesch-Nyhan syndrome, leukodystrophy, Lewy body dementia, lissencephaly, locked-in syndrome, Lou Gehrig's disease, lumbar disc disease, lumbar spinal stenosis, Lyme disease, Machado-Joseph disease (Spinocerebellar ataxia type 3), macrencephaly, macropsia, megalencephaly, Melkersson-Rosenthal syndrome, Meniere's disease, meningitis, Menkes disease, metachromatic leukodystrophy, microcephaly, micropsia, Miller Fisher syndrome, misophonia, mitochondrial myopathy, Mobius syndrome, monomelic amyotrophy, motor neuron disease, motor skills disorder, Moyamoya disease, mucopolysaccharidoses, multi-infarct dementia, multifocal motor neuropathy, multiple sclerosis, multiple system atrophy, muscular dystrophy, myalgic encephalomyelitis, myasthenia gravis, myelinoclastic diffuse sclerosis, myoclonic Encephalopathy of infants, myoclonus, myopathy, myotubular myopathy, myotonia congenital, narcolepsy, neurofibromatosis, neuroleptic malignant syndrome, lupus erythematosus, neuromyotonia, neuronal ceroid lipofuscinosis, Niemann-Pick disease, O'Sullivan-McLeod syndrome, occipital Neuralgia, occult Spinal Dysraphism Sequence, Ohtahara syndrome, olivopontocerebellar atrophy, opsoclonus myoclonus syndrome, optic neuritis, orthostatic hypotension, palinopsia, paresthesia, Parkinson's disease, paramyotonia Congenita, paraneoplastic diseases, paroxysmal attacks, Parry-Romberg syndrome, Pelizaeus-Merzbacher disease, periodic paralyses, peripheral neuropathy, photic sneeze reflex, phytanic acid storage disease, Pick's disease, polymicrogyria (PMG), polymyositis, porencephaly, post-polio syndrome, postherpetic neuralgia (PHN), postural hypotension, Prader-Willi syndrome, primary lateral sclerosis, prion diseases, progressive hemifacial atrophy, progressive multifocal leukoencephalopathy, progressive supranuclear palsy, pseudotumor cerebri, Ramsay Hunt syndrome type I, Ramsay Hunt syndrome type II, Ramsay Hunt syndrome type III, Rasmussen's encephalitis, reflex neurovascular dystrophy, Refsum disease, restless legs syndrome, retrovirus-associated myelopathy, Rett syndrome, Reye's syndrome, rhythmic movement disorder, Romberg syndrome, Saint Vitus dance, Sandhoff disease, schizophrenia, Schilder's disease, schizencephaly, sensory integration dysfunction, septo-optic dysplasia, Shy-Drager syndrome, Sjögren's syndrome, snatiation, Sotos syndrome, spasticity, spina bifida, spinal cord tumors, spinal muscular atrophy, spinocerebellar ataxia, Steele-Richardson-Olszewski syndrome, Stiff-person syndrome, stroke, Sturge-Weber syndrome, subacute sclerosing panencephalitis, subcortical arteriosclerotic encephalopathy, superficial siderosis, Sydenham's chorea, syncope, synesthesia, syringomyelia, tarsal tunnel syndrome, tardive dyskinesia, tardive dysphrenia, Tarlov cyst, Tay-Sachs disease, temporal arteritis, tetanus, tethered spinal cord syndrome, Thomsen disease, thoracic outlet syndrome, tic douloureux, Todd's paralysis, Tourette syndrome, toxic encephalopathy, transient ischemic attack, transmissible spongiform encephalopathies, transverse myelitis, tremor, trigeminal neuralgia, tropical spastic paraparesis, trypanosomiasis, tuberous sclerosis, ubisiosis, Von Hippel-Lindau disease (VHL), Viliuisk Encephalomyelitis (VE), Wallenberg's syndrome, Werdnig, Hoffman disease, west syndrome, Williams syndrome, Wilson's disease, and Zellweger syndrome.

The compounds and compositions may also be useful in the inhibition of the development of invasive cancer, tumor angiogenesis and metastasis.

In some embodiments, the pharmaceutical composition comprises a therapeutically effective amount of a compound of Formula I, or a pharmaceutically acceptable salt thereof, and a pharmaceutically acceptable excipient.

In some embodiments, the disorder or disease is cancer.

In some embodiments, the disorder or disease is metastatic melanoma.

In some embodiments, the disorder or disease is tendon regeneration.

In some embodiments, the disorder or disease is diabetes.

In some embodiments, the disorder or disease is degenerative disc disease.

In some embodiments, the disorder or disease is osteoarthritis.

In some embodiments, the disorder or disease is a viral infection.

In some embodiments, the disorder or disease is a neurological disorder.

In some embodiments, the disorder or disease is Alzheimer's disease.

In some embodiments, the disorder or disease is osteoarthritis.

In some embodiments, the patient is a human.

In some embodiments, the cancer is chosen from: hepatocellular carcinoma, colon cancer, breast cancer, pancreatic cancer, chronic myeloid leukemia (CML), chronic myelomonocytic leukemia, chronic lymphocytic leukemia (CLL), acute myeloid leukemia, acute lymphocytic leukemia, Hodgkin lymphoma, lymphoma, sarcoma, and ovarian cancer.

In some embodiments, the cancer is chosen from: lung cancer—non-small cell, lung cancer—small cell, multiple myeloma, nasopharyngeal cancer, neuroblastoma, osteosarcoma, penile cancer, pituitary tumors, prostate cancer, retinoblastoma, synovial sarcoma, rhabdomyosarcoma, salivary gland cancer, skin cancer—basal and squamous cell, skin cancer—melanoma, small intestine cancer, stomach (gastric) cancers, testicular cancer, thymus cancer, thyroid cancer, uterine sarcoma, vaginal cancer, vulvar cancer, laryngeal or hypopharyngeal cancer, kidney cancer, Kaposi sarcoma, gestational trophoblastic disease, gastrointestinal stromal tumor, gastrointestinal carcinoid tumor, gallbladder cancer, eye cancer (melanoma and lymphoma), Ewing tumor, esophagus cancer, endometrial cancer, colorectal cancer, cervical cancer, brain or spinal cord tumor, bone metastasis, bone cancer, bladder cancer, bile duct cancer, anal cancer and adrenal cortical cancer.

In some embodiments, the cancer is hepatocellular carcinoma; in some embodiments, the cancer is colon cancer; in some embodiments, the cancer is colorectal cancer; in some embodiments, the cancer is breast cancer; in some embodiments, the cancer is pancreatic cancer; in some embodiments, the cancer is chronic myeloid leukemia (CML); in some embodiments, the cancer is chronic myelomonocytic leukemia; in some embodiments, the cancer is chronic lymphocytic leukemia (CLL); in some embodiments, the cancer is acute myeloid leukemia; in some embodiments, the cancer is acute lymphocytic leukemia; in some embodiments, the cancer is Hodgkin lymphoma; in some embodiments, the cancer is lymphoma; in some embodiments, the cancer is sarcoma; in some embodiments, the cancer is ovarian cancer; in some embodiments, the cancer is lung cancer—non-small cell; in some embodiments, the cancer is lung cancer—small cell; in some embodiments, the cancer is multiple myeloma; in some embodiments, the cancer is nasopharyngeal cancer; in some embodiments, the cancer is neuroblastoma; in some embodiments, the cancer is osteosarcoma; in some embodiments, the cancer is penile cancer; in some embodiments, the cancer is pituitary tumors; in some embodiments, the cancer is prostate cancer; in some embodiments, the cancer is retinoblastoma; in some embodiments, the cancer is rhabdomyosarcoma; in some embodiments, the cancer is salivary gland cancer; in some embodiments, the cancer is skin cancer—basal and squamous cell; in some embodiments, the cancer is skin cancer—melanoma; in some embodiments, the cancer is small intestine cancer; in some embodiments, the cancer is stomach (gastric) cancers; in some embodiments, the cancer is testicular cancer; in some embodiments, the cancer is thymus cancer; in some embodiments, the cancer is thyroid cancer; in some embodiments, the cancer is uterine sarcoma; in some embodiments, the cancer is vaginal cancer; in some embodiments, the cancer is vulvar cancer; in some embodiments, the cancer is Wilms tumor; in some embodiments, the cancer is laryngeal or hypopharyngeal cancer; in some embodiments, the cancer is kidney cancer; in some embodiments, the cancer is Kaposi sarcoma; in some embodiments, the cancer is gestational trophoblastic disease; in some embodiments, the cancer is gastrointestinal stromal tumor; in some embodiments, the cancer is gastrointestinal carcinoid tumor; in some embodiments, the cancer is gallbladder cancer; in some embodiments, the cancer is eye cancer (melanoma and lymphoma); in some embodiments, the cancer is Ewing tumor; in some embodiments, the cancer is esophagus cancer; in some embodiments, the cancer is endometrial cancer; in some embodiments, the cancer is colorectal cancer; in some embodiments, the cancer is cervical cancer; in some embodiments, the cancer is brain or spinal cord tumor; in some embodiments, the cancer is bone metastasis; in some embodiments, the cancer is bone cancer; in some embodiments, the cancer is bladder cancer; in some embodiments, the cancer is bile duct cancer; in some embodiments, the cancer is anal cancer; and in some embodiments, the cancer is adrenal cortical cancer.

In some embodiments, the disorder or disease is a neurological condition, disorder, or disease, wherein the neurological disease is selected from: Alzheimer's disease, frontotemporal dementias, Parkinson's disease, Huntington's disease, progressive supranuclear palsy, corticobasal degeneration, multiple system atrophy, amyotrophic lateral sclerosis (ALS), inclusion body myositis, autism, degenerative myopathies.

In some embodiments, the disorder or disease is selected from the group consisting of: Alzheimer's disease, amyotrophic lateral sclerosis, Down syndrome, frontotemporal dementia with parkinsonism-17 (FTDP-17), Lewy body dementia, Parkinson's disease, Pick's disease, and additional diseases with pronounced neurodegeneration such as autism, dementia, epilepsy, Huntington's disease, multiple sclerosis; diseases and disorders associated with acquired brain injury such as chronic traumatic encephalopathy, traumatic brain injury, tumor, and stroke.

In some embodiments, a compound of Formula I inhibits DYRK1A.

In some embodiments, the method treats a disease or disorder mediated by kinase activity in a patient, the method comprises administering to the patient a therapeutically effective amount of a compound (or compounds) of Formula I, or a pharmaceutically acceptable salt thereof.

In some embodiments, the disease or disorder comprises tumor growth, cell proliferation, or angiogenesis.

In some embodiments, the method inhibits the activity of a protein kinase receptor, the method comprises contacting the receptor with an effective amount of a compound (or compounds) of Formula I, or a pharmaceutically acceptable salt thereof.

In some embodiments, the method treats a disease or disorder associated with aberrant cellular proliferation in a patient; the method comprises administering to the patient a therapeutically effective amount of a compound (or compounds) of Formula I, or a pharmaceutically acceptable salt thereof.

In some embodiments, the method prevents or reduces abnormal cellular proliferation in a patient; the method comprises administering to the patient a therapeutically effective amount of a compound (or compounds) of Formula I, or a pharmaceutically acceptable salt thereof.

In some embodiments, the method treats a disease or disorder associated with aberrant cellular proliferation in a patient, the method comprises administering to the patient a pharmaceutical composition comprising one or more of the compounds of claim 1 in combination with a pharmaceutically acceptable carrier and one or more other agents.

### Evaluation of Biological Activity

The biological activity of the compounds described herein can be tested using any suitable assay known to those of skill in the art. For example, the activity of a compound may be tested using one or more of the test methods outlined below.

For example, in vitro assays for DYRK1A biological activity may be used, e.g., regulation of microtubule-associated protein tau (MAPT/Tau) phosphorylation in neuronal cell lines such as the human SH-SY5Y neuroblastoma cell line. Assays for DYRK1A-regulated level of phosphorylation can include monitoring levels of basal pSer396 Tau, which can be measured, for example, by serial dilutions of a candidate inhibitor composition using a ten micromolar top concentration and detected by ELISA or Western Blotting. An exemplary assay for DYRK-1A-regulated phosphorylation uses the SH-SY5Y cells cultured in a 96 well plate format for a period of time sufficient to stabilize microtubules and Tau phosphorylation, usually at least 2 days, then treated with a 1/3 serial dilution of compounds overnight and lysed. The cell lysate is resolved by SDS PAGE, then transferred to nitrocellulose and probed with an antibody specific for pSer396 Tau. The chemiluminescence signal for HRP-linked antibodies used in western blotting is detected using a Carestream Image Station and blot densitometry for pSer396 and beta-actin are analyzed using ImageJ (NIH).

In a further example, the activity of a candidate compound can be measured by phosphoTau (Thr212) AlphaLISA by adding the lysate mentioned above onto total Tau-coated plates and detected with a specific pThr212Tau antibody. Colorimetric detection of AlphaLISA signal is performed by EnVision Multilabel Plate Reader (Perkin Elmer).

To further illustrate this disclosure, the following examples are included. The examples should not, of course, be construed as specifically limiting the disclosure. Variations of these examples within the scope of the claims are within the purview of one skilled in the art and are considered to fall within the scope of the disclosure as described and claimed herein. The reader will recognize that the skilled artisan, armed with the present disclosure, and skill in the art is able to prepare and use the disclosure without exhaustive examples.

### EXAMPLES

### Compound Preparation

The starting materials used in preparing the compounds of the disclosure are known, made by known methods, or are commercially available. It will be apparent to the skilled artisan that methods for preparing precursors and functionality related to the compounds claimed herein are generally described in the literature. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry: Reactions, Mechanisms, and Structure 7^{th} Ed., John Wiley & Sons (2013), Carey and Sundberg, Advanced Organic Chemistry 5^{th} Ed., Springer (2007), Comprehensive Organic Transformations: A Guide to Functional Group Transformations, 2^{nd} Ed., John Wiley & Sons (1999) (incorporated herein by reference in its entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in P. Wuts Greene's Protective Groups in Organic Synthesis, 5th Ed., John Wiley & Sons (2014), incorporated herein by reference in its entirety.

Trademarks used herein are examples only and reflect illustrative materials used at the time of the disclosure. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the disclosure.

(¹H) nuclear magnetic resonance spectra (NMR) were measured in the indicated solvents on a Bruker NMR spectrometer (Avance™ DRX300, 300 MHz for ¹H or Avance™ DRX500, 500 MHz for ¹H) or Varian NMR spectrometer (Mercury 400BB, 400 MHz for ¹H). Peak positions are expressed in parts per million (ppm) downfield from tetramethylsilane. The peak multiplicities are denoted as follows, s, singlet; d, doublet; t, triplet; q, quartet; ABq, AB quartet; quin, quintet; sex, sextet; sep, septet; non, nonet; dd, doublet of doublets; ddd, doublet of doublets of doublets; d/ABq, doublet of AB quartet; dt, doublet of triplets; td, triplet of doublets; dq, doublet of quartets; m, multiplet.

The following abbreviations have the indicated meanings:
Ac₂O=acetic anhydride
Boc=tert-butyloxycarbonyl
BrettPhos=dicyclohexyl(2′,4′,6′-triisopropyl-3,6-dimethoxy-[1,1′-biphenyl]-2-yl) phosphine
BrettPhos Pd G3=[(2-di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate
brine=saturated aqueous sodium chloride
nBuLi=n-butyl lithium
CBzC1=benzyl chloroformate
CDCl₃=deuterated chloroform
SFC=supercritical fluid chromatography
Cs₂CO₃=cesium carbonate
DCE=1,2-dichloroethane
DCM=dichloromethane
DIPEA=N,N-diisopropylethylamine
DME=1,2-dimethoxyethane, or glyme, or monoglyme
DMF=N,N-dimethylformamide
DMSO-d₆=deuterated dimethylsulfoxide
EDCI=1-Ethyl-3-(3-dimethylaminopropyl)carbodiimide
ESIMS=electron spray mass spectrometry
EtOAc=ethyl acetate
HATU=1-[bis(dimethylamino)methylene]-1H-1,2,3-triazolo[4,5-b]pyridinium 3-oxid
hexafluorophosphate
HCl=hydrochloric acid
HOAc=acetic acid
HOBt=1-hydroxybenzotriazole
ISCO=Teledyne ISCO, Inc brand CombiFlash® Rf 200
KHMDS=potassium bis(trimethylsilyl)amide
KOAc=potassium acetate
LCMS=liquid chromatography-mass spectrometry
LiHMDS=lithium bis(trimethylsilyl)amide
MCPBA=meta-chloroperoxybenzoic acid
MeCN=acetonitrile
MeOH=methanol
MgSO₄=magnesium sulfate
MW=microwave irradiation
NaO^{t}Bu=sodium tert-butoxide
NaHCO₃=sodium bicarbonate
NaBH(OAc)₃=Sodium triacetoxyborohydride
Na₂SO₄=sodium sulfate
NMM=4-Methylmorpholine
NMR=nuclear magnetic resonance
ON=overnight
Pd/C=palladium on carbon
Pd(dppf)Cl₂=1,1′-bis(diphenylphosphino)ferrocene-palladium(I1)dichloride
Pd(OAc)₂=palladium(II) acetate
PE=petroleum ether
QPhos=1,2,3,4,5-pentaphenyl-1′-(di-tert-butylphosphino)ferrocene
r.t.=room temperature
SFC=supercritical fluid chromatography
TEA=triethylamine
TFA=trifluoroacetic acid
THF=tetrahydrofuran
TLC=thin layer chromatography
TMSOK=potassium trimethylsilanolate
pTsOH=para-toluenesulfonic acid
XPhos=dicyclohexyl[2′,4′,6′-tris(propan-2-yl)[1,1′-biphenyl]-2-yl]phosphane

The following example schemes are provided for the guidance of the reader, and collectively represent an example method for making the compounds provided herein. Furthermore, other methods for preparing compounds of the disclosure will be readily apparent to the person of ordinary skill in the art in light of the following reaction schemes and examples. The skilled artisan is thoroughly equipped to prepare these compounds by those methods given the literature and this disclosure. The compound numberings used in the synthetic schemes depicted below are meant for those specific schemes only and should not be construed as or confused with same numberings in other sections of the application. Unless otherwise indicated, all variables are as defined above.

### General Procedures

Compounds of Formula I of the present disclosure can be prepared as depicted in Scheme 1.

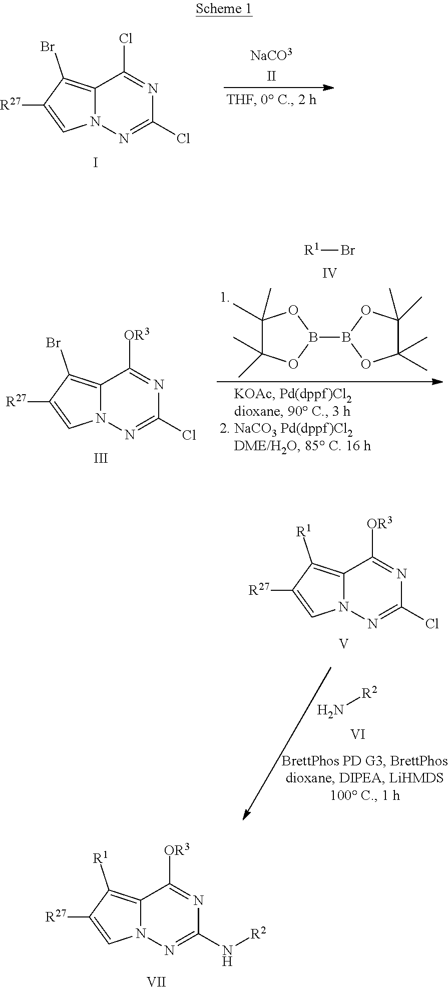

Scheme 1 describes a method for preparation of 4-alkoxypyrrolo[2,1-f][1,2,4]triazine derivatives (VII) by first displacing the 4-chloride (I) with a variety of alkoxys (II) to produce 2-chloro-5-bromo-4-alkoxypyrrolo[2,1-f][1,2,4]triazine III. Formation of a variety of boronic acid pinacol esters by reacting various bromides (IV) with bis(pinacolato)diboron followed by Suzuki coupling with bromide (III) produces 2-chloro-4-alkoxypyrrolo[2,1-f][1,2,4]triazine (V). The chloro is then displaced with a variety of amines (VI) to produce the final 4-alkoxypyrrolo[2,1-f][1,2,4]triazine (VII).

### Illustrative Compound Examples

Preparation of intermediate 5-bromo-2-chloro-4-methoxypyrrolo[2,1-f][1,2,4]triazine (XII) is depicted below in Scheme 2.

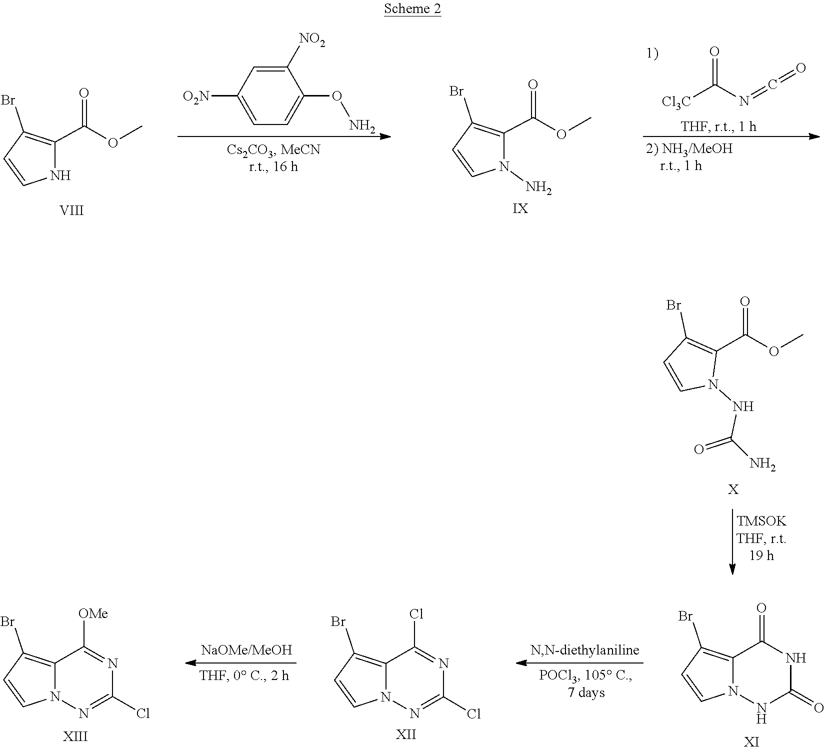

### Step 1

To a solution of methyl 3-bromo-1H-pyrrole-2-carboxylate (VIII) (100 g, 490.15 mmol, 1 eq.) in MeCN (2 L) was added Cs₂CO₃ (255.52 g, 784.23 mmol, 1.6 eq.) in one portion at 20° C. (no exothermic). The reaction was stirred at room temperature for 5 h. A solution of O-(2,4-dinitrophenyl)hydroxylamine (146.40 g, 735.22 mmol, 1.5 eq.) in MeCN (2 L) was then added dropwise at 0-5° C. under N₂. After addition, the reaction was warmed to room temperature for 16 h. The reaction mixture was filtered, and the filtrate was diluted with MTBE (2 L) and washed with brine (2 L×3). The above reaction was performed six times.

The combined organics of five batches were combined, dried over MgSO₄, filtered, and concentrated under vacuum to give the crude product. The crude product was purified by column chromatography on silica gel (DCM/PE=0-50%, then EtOAc/PE=20%) to give methyl 1-amino-3-bromo-1H-pyrrole-2-carboxylate (IX) (380 g, 1.62 mol, 55.1% yield, 93.3% purity) as a yellow solid. ¹H NMR (400 MHz, DMSO-d₆) δ ppm 3.84 (3H, s), 6.24 (1H, d, J=2.8 Hz), 6.38 (2H, br s), 7.12 (1H, d, J=2.8 Hz).

### Steps 2-3

To a solution of methyl 1-amino-3-bromo-1H-pyrrole-2-carboxylate (IX) (190 g, 867.44 mmol, 1 eq.) in THF (1900 mL) was added dropwise 2,2,2-trichloroacetyl isocyanate (179.76 g, 954.18 mmol, 113.06 mL, 1.1 eq.) at 0˜5° C., then warmed to room temperature for 1 h. The reaction was then added dropwise to NH₃/MeOH (7 M, 1.24 L, 10 eq) at room temperature, stirred at room temperature for 1 h to give a yellow suspension. The above reaction was performed twice.

The two reactions were combined and concentrated under vacuum. The residue was triturated with MTBE (4 L) at room temperature for 30 min, filtered. The filter cake was washed with MTBE (500 mL×3), dried under vacuum to give methyl 3-bromo-1-ureido-1H-pyrrole-2-carboxylate (X) (402 g, 1.53 mol, 88.2% yield, 99.78% purity) as an off-white solid. ¹H NMR (400 MHz, DMSO-d₆) δ ppm 3.75 (3H, s), 6.27 (2H, br s), 6.28 (1H, d, J=2.8 Hz), 7.10 (1H, d, J=2.8 Hz), 9.27 (1H, br s); ESIMS found for C₇H₈BrN₃O₃ m/z 262.1 (⁷⁹BrM+H).

### Step 4

To a solution of methyl 3-bromo-1-ureido-1H-pyrrole-2-carboxylate (X) (67 g, 255.67 mmol, 1 eq.) in THF (4 L) was added TMSOK (65.60 g, 511.33 mmol, 2 eq.) in portions at 0˜5° C. under N₂. After stirring for 5 min, the reaction became a thick slurry. The reaction mixture was stirred vigorously at room temperature for 16 h. LCMS showed ˜25.8% of starting material remained. An additional portion of TMSOK (16.40 g, 127.83 mmol, 0.5 eq.) was added at room temperature and stirred for 3 h. LCMS showed the reaction was complete. The reaction was concentrated. The residue was diluted with H₂O (1.6 L), cooled with ice-bath, added dropwise HCl (4 M, 160 mL) to pH 2-3, and filtered. The filter cake was washed with H₂O (600 mL×3). The above reaction was performed six times.

The filter cakes of six reactions were combined, triturated with H₂O (6 L) at room temperature, for 2 h and then filtered. The filter cake was dried in a vacuum drying oven at 60° C. for 24 h to give 5-bromopyrrolo[2,1-f][1,2,4]triazine-2,4(1H,3H)-dione (XI) (320 g, 1.28 mol, 83.4% yield, 91.95% purity) as a white solid. ¹H NMR (400 MHz, DMSO-d₆) δ ppm 6.49 (1H, d, J=2.8 Hz), 7.17 (1H, d, J=2.8 Hz), 11.30 (1H, br s); ESIMS found for C₆H₄BrN₃O₂ m/z 232.0 (⁸¹BrM+H).

### Step 5

To a solution of POCl₃ (1500 mL) was added 5-bromopyrrolo[2,1-f][1,2,4]triazine-2,4(1H,3H)-dione (XI) (180 g, 782.55 mmol, 1 eq.) in portions at room temperature. N,N-Diethylaniline (291.95 g, 1.96 mol, 312.92 mL, 2.5 eq.) was then added dropwise at room temperature (a little exothermic). After addition, the reaction was heated to 105° C. for 48 h. LCMS showed the starting material was consumed but the intermediate remained, ˜4.3% of desired product was formed. The reaction was cooled to 90° C., added an additional POCl₃ (500 mL) in one portion, then heated to reflux gently for 24 h. LCMS showed most of intermediate remained, ˜11.9% of desired product was formed. The reaction was heated to reflux gently for another 4 days. LCMS showed ˜7.4% of intermediate remained, ˜55.5% of desired product was formed. The reaction was cooled to 40° C. and distilled under reduced pressure to remove most of POCl₃. The residue was diluted with MeTHF (5 L), poured into ice-H₂O (2 L), the added brine (1 L), and separated. The aqueous layer was extracted with MeTHF (1.5 L×2). The combined organic were washed with brine (1.5 L×2), dried over MgSO₄, filtered, and concentrated under vacuum to give the crude product. The crude product was purified by column chromatography on silica gel (4 kg, 100˜200 mesh, DCM/PE=0-20%) and triturated with n-heptane (500 mL) at room temperature for 3 h, then filtered to afford 5-bromo-2,4-dichloropyrrolo[2,1-f][1,2,4]triazine (XII) (150 g, 556.14 mmol, 71.1% yield, 98.96% purity) as a yellow solid. ¹H NMR (400 MHz, CDCl₃) δ ppm 7.02 (1H, d, J=2.4 Hz), 7.79 (1H, d, J=2.4 Hz); ESIMS found for C₆H₂BrCl₂N₃ m/z 266.1 (⁷⁹BrM+H).

### Step 6

To a stirred solution of 5-bromo-2,4-dichloropyrrolo[2,1-f][1,2,4]triazine (XII) (8.0 g, 29.97 mmol) in THF (10 mL) was added sodium methoxide (66 mL, 33 mmol) in MeOH dropwise at 0° C. and the mixture was stirred for 2 h. The reaction mixture was quenched with saturated aqueous NH₄Cl, taken into EtOAc, washed with water and brine. The organics were separated, dried over anhydrous Na₂SO₄, concentrated, and dried under high vacuo to obtain 5-bromo-2-chloro-4-methoxypyrrolo[2,1-f][1,2,4]triazine (XIII) (6 g, 22.86 mmol, 76.3% yield) as an off-white solid which was used for the next step without purification. ¹H NMR (499 MHz, DMSO-d₆) δ ppm 4.12 (3H, s), 7.00 (1H, d, J=3.01 Hz), 8.01 (1H, d, J=3.01 Hz); ESIMS found for C₇H₅BrClN₃O m/z 261.1 (⁷⁹BrM+H).

The following intermediate was prepared in accordance with the procedure described in the above Scheme 2.

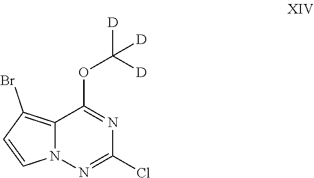 [SMILES: [2H]C([2H])([2H])Oc1nc(Cl)nn2ccc(Br)c12]

5-Bromo-2-chloro-4-(methoxy-d₃)pyrrolo[2,1-f][1,2,4]triazine (XIV): Light yellow solid (856 mg, 3.224 mmol, 81.2% yield). ¹H NMR (499 MHz, DMSO-d₆) δ ppm ¹H NMR (499 MHz, DMSO-d₆) δ ppm 7.00 (1H, d, J=3.01 Hz), 8.01 (1H, d, J=3.01 Hz); ESIMS found C₇H₂D₃BrClN₃O m/z 264.9 (M+H).

Preparation of intermediate 5-bromo-2-chloro-6-fluoro-4-methoxypyrrolo[2,1-f][1,2,4]triazine (XXIV) is depicted below in Scheme 3.

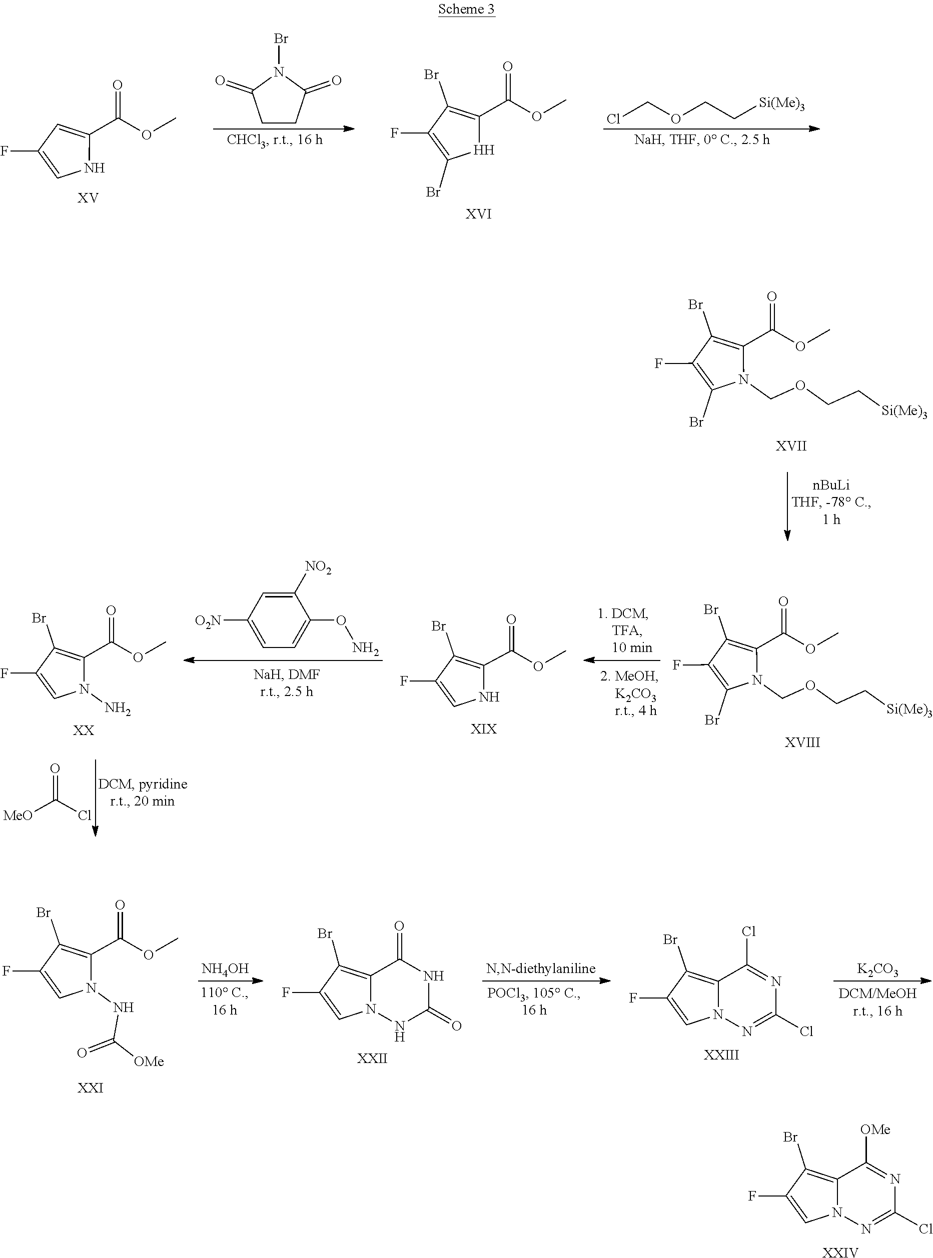

### Step 1

To a solution of methyl 4-fluoro-1H-pyrrole-2-carboxylate (XV) (commercially available from PharmaBlock (USA), Inc.) (1 g, 6.99 mmol) in CHCl₃ (9 mL) at 0° C. was added N-bromosuccinimide (2.64 g, 14.68 mmol). The reaction was stirred at room temperature for 16 h. The reaction mixture was quenched with 10% sodium thiosulfate (50 mL) and extracted with CHC₃ (3×30 mL). The organic layers were combined and washed with aqueous saturated NaCl (50 ml) and evaporated under reduced pressure to produce methyl 3,5-dibromo-4-fluoro-1H-pyrrole-2-carboxylate (XVI) (2.1 g, 6.979 mmol, 99.9% yield) as a beige solid. The product was used as is without further purification. ESIMS found for C₆H₄Br₂FNO₂ m/z 299.9 (M+H).

### Step 2

To a solution of methyl 3,5-dibromo-4-fluoro-1H-pyrrole-2-carboxylate (XVI) (2.1 g, 6.98 mmol) in dry THF (25 mL) at 0° C. under N₂ was added slowly NaH (0.43 g, 10.63 mmol) in small portions. The suspension was stirred at 0° C. for 30 min. To the suspension was added (2-(chloromethoxy)ethyl)trimethylsilane (1.9 mL, 10.74 mmol) at 0° C. under N₂. The reaction mixture was stirred at 0° C. for 2 h before quenching with ice-water (20 mL) and extracted with EtOAc (3×30 mL). The EtOAc layer was evaporated under reduced pressure and purified by silica gel column chromatography (100% hexanes) to produce methyl 3,5-dibromo-4-fluoro-1-((2-(trimethylsilyl)ethoxy)methyl)-1H-pyrrole-2-carboxylate (XVII) (2.76 g, 6.401 mmol, 91.7% yield) as an amber liquid. ¹H NMR (499 MHz, DMSO-d₆) δ ppm −0.07 (9H, s), 0.81 (2H, t, J=7.80 Hz), 3.50 (2H, t, J=7.80 Hz), 3.82 (3H, s), 5.69 (2H, s).

### Step 3

To solution of methyl 3,5-dibromo-4-fluoro-1-((2-(trimethylsilyl)ethoxy) methyl)-1H-pyrrole-2-carboxylate (XVII) (2.76 g, 6.4 mmol) in dry THF (60 mL) at −78° C. under N₂ was added nBuLi (2.4 mL, 6 mmol). The reaction mixture was stirred at −78° C. for 1 h. The mixture was carefully quenched with ice water (20 mL), warmed to room temperature, and extracted with EtOAc (3×20 mL). The organics were dried over anhydrous Na₂SO₄, filtered and evaporated onto Celite® and purified by silica gel column chromatography (0→6% EtOAc/hexanes) to produce methyl 3-bromo-4-fluoro-1-((2-(trimethylsilyl)ethoxy)methyl)-1H-pyrrole-2-carboxylate (XVIII) (760 mg, 2.157 mmol, 33.7% yield) as a clear oil. ¹HNMR (500 MHz, DMSO-d₆) δ ppm −0.06 (9H, s), 0.80 (2H, t, J=7.96 Hz), 3.41-3.50 (2H, m), 3.80 (3H, s), 5.56 (2H, s), 7.52 (1H, d, J=3.29 Hz).

### Step 4

To a stirred solution of methyl 3-bromo-4-fluoro-1-((2-(trimethylsilyl) ethoxy)methyl)-1H-pyrrole-2-carboxylate (XVIII) (881 mg, 2.5 mmol) in DCM (23 mL) was added TFA (23 mL) and the mixture was stirred at room temperature for 10 min. The solvents were concentrated, the residue taken in a mixture of MeOH (12 mL)/water (6 mL). To this mixture was added K₂CO₃ (1.75 g, 12.66 mmol) and stirred at room temperature for 4 h. Water (10 mL) was added, and the solution was neutralized with 1H HCl and washed with CHCl₃ (3×10 mL). The organic layer was stripped onto Celite® and purified by silica gel column chromatography (0→5% MeOH/CHCl₃ (7 N NH₃)) to produce methyl 3-bromo-4-fluoro-1H-pyrrole-2-carboxylate (XIX) (450 mg, 2.027 mmol, 81.0% yield) as an off white solid. ¹H NMR (499 MHz, DMSO-d₆) δ ppm 3.80 (3H, s), 7.15 (1H, d, J=3.56 Hz), 12.29 (1H, br s); ESIMS found for C₆HsBrFNO₂ m/z 222.0 (M+H).

### Step 5

To a solution of methyl 3-bromo-4-fluoro-1H-pyrrole-2-carboxylate (XIX) (500 mg, 2.25 mmol) in dry DMF (7 mL) at 0° C. was slowly added NaH (120 mg, 2.93 mmol). After 30 min, 0-(2,4-dinitrophenyl)hydroxylamine (490 mg, 2.48 mmol)) was added in one portion and the reaction was stirred in the ice bath for 1 h and at room temperature for 1 h. LC/MS showed the reaction was incomplete so addition O-(2,4-dinitrophenyl)hydroxylamine (490 mg, 2.48 mmol) and stirred at room temperature for 15 min. The reaction was diluted with water (100 mL) and extracted with EtOAc (3×75 mL). The combined extracts were stripped onto Celite® and purified by silica gel column chromatography (100% CHCl₃) to produce methyl 1-amino-3-bromo-4-fluoro-1H-pyrrole-2-carboxylate (XX) (501 mg, 2.114 mmol, 93.9% yield) as an off-white solid. ¹H NMR (499 MHz, DMSO-d₆) δ ppm 3.80 (3H, s), 6.36 (2H, s), 7.25 (1H, d, J=3.29 Hz); ESIMS found for C₆H₆BrFN₂O₂ m/z 237.0 (M+H).

### Step 6

To a solution of methyl 1-amino-3-bromo-4-fluoro-1H-pyrrole-2-carboxylate (XX) (500 mg, 2.11 mmol) in DCM (10 mL) was added pyridine (210 μL, 2.6 mmol) and methyl carbonochloridate (180 μL, 2.33 mmol) dropwise. The reaction was stirred at room temperature for 20 min. DCM (100 mL) was added and washed with 1 N HCl (2×50 mL), stripped onto Celite® and purified by silica gel column chromatography (0→100% EtOAc/hexanes) to produce methyl 3-bromo-4-fluoro-1-((methoxycarbonyl) amino)-1H-pyrrole-2-carboxylate (XXI) (560 mg, 1.898 mmol, 89.8% yield) as a clear oil. ¹H NMR (499 MHz, DMSO-d₆) δ ppm 3.69 (3H, br s), 3.76 (3H, s), 7.48 (1H, br d, J=1.92 Hz), 10.76 (1H, br s); ESIMS found for C₈H₈BrFN₂O₄ m/z 295.0 (M+H).

### Step 7

To NH₄OH (35 mL, 263.7 mmol) in a sealed tube was added methyl 3-bromo-4-fluoro-1-((methoxycarbonyl)amino)-1H-pyrrole-2-carboxylate (XXI) (560 mg, 1.9 mmol). The reaction was sealed and heated to 110° C. for 16 h. The solvent was removed under vacuum and the residue was dissolved in MeCN. The solvent was stripped (3× to remove water) and placed under high vacuum for 1 h, to produce 5-bromo-6-fluoropyrrolo[2,1-f][1,2,4]triazine-2,4(1H,3H)-dione (XXII) (440 mg, 1.774 mmol, 93.5% yield) as a light brown solid. The product was used as is without further purification. ¹H NMR (499 MHz, DMSO-d₆) δ ppm 7.04 (1H, d, J=3.29 Hz), 7.13 (1H, br s), 9.58 (1H, br s); ESIMS found for C₆H₃BrFN₃O₂ m/z 247.9 (M+H).

### Step 8

To a solution of neat POCl₃ (3 mL, 32.19 mmol) was added 5-bromo-6-fluoropyrrolo[2,1-f][1,2,4]triazine-2,4(1H,3H)-dione (XXII) (440 mg, 1.77 mmol) in portions at room temperature. N,N-Diethylaniline (0.7 mL, 4.4 mmol) was then added dropwise at room temperature. The reaction was heated at 105° C. for 16 h. The solvent was removed under vacuum before adding water (20 mL) and extracting with EtOAc. The organic layer was stripped onto Celite® and purified by silica gel column chromatography (0→100% EtOAc/hexanes) to produce 5-bromo-2,4-dichloro-6-fluoropyrrolo[2,1-f][1,2,4]triazine (XXIII) (175 mg, 0.614 mmol, 34.6% yield) as an off-white solid. ¹H NMR (499 MHz, DMSO-d₆) δ ppm 7.92 (1H, d, J=3.29 Hz); ESIMS found for C₆HBrCl₂FN₃ m/z 283.9 (M+H).

### Step 9

To a solution of 5-bromo-2,4-dichloro-6-fluoropyrrolo[2,1-f][1,2,4]triazine (XXIII) (175 mg, 0.61 mmol) in MeOH (664 μL) and DCM (2 mL) was added K₂CO₃ (254.1 mg, 1.84 mmol). The flask was capped and the reaction and stirred at room temperature for 16 h. Water (50 mL) was added and extracted with DCM (3×50 mL). The organic layers were separated and washed with water (2×50 mL) and brine (50 mL). The solvent was removed under vacuum to produce 5-bromo-2-chloro-6-fluoro-4-methoxypyrrolo[2,1-f][1,2,4]triazine (XXIV) (163 mg, 0.581 mmol, 94.6% yield) as a beige solid. ¹H NMR (500 MHz, DMSO-d₆) δ ppm 4.12 (3H, s), 8.34 (1H, d, J=3.29 Hz); ESIMS found for C₇H₄BrClFN₃O m/z 279.95 (M+H).

Preparation of intermediate 2,5-dichloro-4-methoxypyrrolo[2,1-f][1,2,4]triazine (XXVI) is depicted below in Scheme 4.

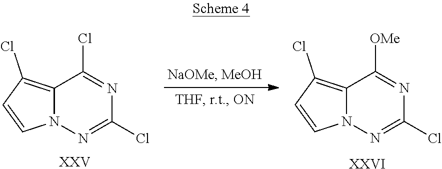

### Step 1

To a stirred solution of 2,4,5-trichloropyrrolo[2,1-f][1,2,4]triazine (XXV) (commercially available from PharmaBlock Sciences Inc.) (202 mg, 0.910 mmol) in THF (4 mL) was added sodium methoxide (2 mL, 1 mmol) in MeOH dropwise at 0° C. and the mixture was stirred at room temperature overnight. The reaction mixture was quenched with water, taken into EtOAc, and washed with brine. The organics were separated, dried over anhydrous Na₂SO₄, concentrated, and the crude mixture was purified on ISCO (0→100% EtOAc/hexanes). Fractions were collected and concentrated under reduced pressure and dried under high vacuo to obtain 5-bromo-2-chloro-4-methoxypyrrolo[2,1-f][1,2,4]triazine (XXVI) (140 mg, 0.642 mmol, 70.7% yield) as a pale-yellow solid. ¹H NMR (499 MHz, DMSO-d₆) δ ppm 4.12 (3H, s), 6.95 (1H, d, J=3.01 Hz), 8.02 (1H, d, J=3.01 Hz).

Preparation of intermediate 6-bromo-1-(2,2-difluoroethyl)-4-fluoro-2-methyl-1H-benzo[d]imidazole (XXXI) is depicted below in Scheme 5.

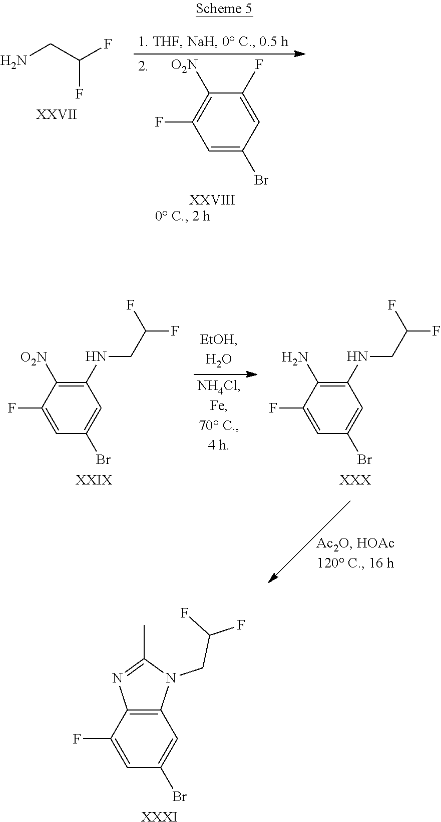

### Step 1

To an ice-cold suspension of sodium hydride (222 mg, 9.2 mmol) in THF (20 mL) was added 2,2-difluoroethylamine (XXVII) (746 mg, 9.32 mmol). The reaction mixture was allowed to stir at 0° C. for 30 min before adding an ice-cold solution of 5-bromo-1,3-difluoro-2-nitrobenzene (XXVIII) (2.0 g, 8.4 mmol) in THF (20 mL). Reaction mixture was stirred at 0° C. for 2 h. The reaction was quenched with water. The aqueous layer was extracted with EtOAc, the organics were washed with brine, dried over anhydrous Na₂SO₄, filtered and concentrated. The crude product was purified via column chromatography (40 g of silica gel) (0→20% EtOAc/hexanes) to give 5-bromo-N-(2,2-difluoroethyl)-3-fluoro-2-nitroaniline (XXIX) (500 mg, 0.836 mmol, 19.9% yield) as a yellow solid. ESIMS found for C₈H₆BrF₃N₂O₂ m/z 299.0 (⁷⁹BrM+H).

### Step 2

A mixture of 5-bromo-N-(2,2-difluoroethyl)-3-fluoro-2-nitroaniline (XXIX) (500 mg, 1.67 mmol), Fe (1.09 g, 16.72 mmol) and NH₄Cl (1.34 g, 25.1 mmol) in a mixture of EtOH (6 mL) and water (2.5 mL) was heated to 70° C. for 4 h. The reaction mixture was cooled and filtered through Celite®. Filtrates were dissolved in EtOAc, washed water and brine, dried over anhydrous Na₂SO₄, filtered, and concentrated under reduced pressure to obtain 5-bromo-N¹-(2,2-difluoroethyl)-3-fluorobenzene-1,2-diamine (XXX) (420 mg, 1.561 mmol, 93.4% yield) as a brown solid which was used for next reaction without further purification. ESIMS found for C₈H₈BrF₃N₂ m/z 269.0 (M+H).

### Step 3

A solution of 5-bromo-N¹-(2,2-difluoroethyl)-3-fluorobenzene-1,2-diamine (XXX) (420 mg, 1.56 mmol) and Ac₂O (175 μL, 1.85 mmol) in HOAc (8 mL) was heated to 120° C. for 16 h. The reaction mixture was concentrated, the residue partitioned between EtOAc/1 N NaOH, organics separated and washed with water and brine. The organics were dried over anhydrous Na₂SO₄ and concentrated to yield 6-bromo-1-(2,2-difluoroethyl)-4-fluoro-2-methyl-1H-benzo[d]imidazole (XXXI) (380 mg, 1.297 mmol, 83.1% yield) as a light brown solid. ESIMS found for C₁₀H₁₀BrF₃N₂ m/z 295.0 (M+H).

Preparation of intermediate 6-bromo-1-(2,2-difluoroethyl)-2-methyl-1H-benzo[d]imidazole (XXXV) is depicted below in Scheme 6.

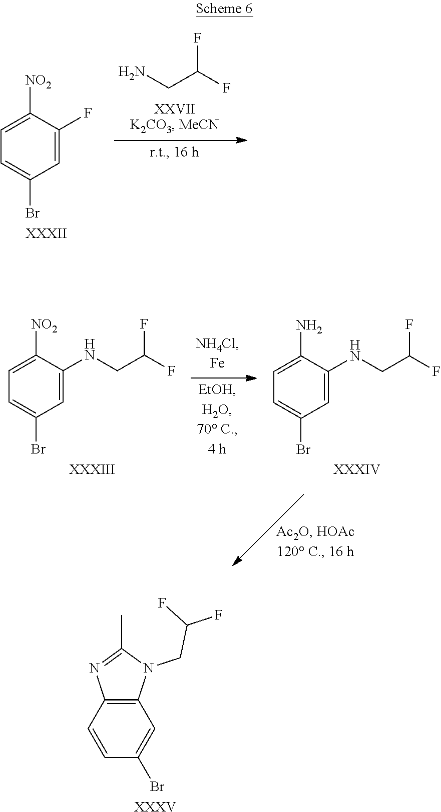

### Step 1

A mixture of K₂CO₃ (1.66 g, 11.99 mmol), 2,2-difluoroethylamine (XXVII) (0.49 g, 6.05 mmol) and 4-bromo-2-fluoro-1-nitrobenzene (XXXII) (1.2 g, 5.45 mmol) in MeCN (20 mL) was stirred at room temperature for 16 h. The reaction was filtered, solvents concentrated, and purified via column chromatography (24 g of silica gel) (0-20% EtOAc/hexanes) to yield 5-bromo-N-(2,2-difluoroethyl)-2-nitroaniline (XXXIII) (1.33 g, 4.73 mmol, 86.8% yield) as a white solid which was used in the next step without further purification. ESIMS found for C₈H₇BrF₂N₂O₂ m/z 281.0 (M+H).

### Step 2

A mixture of 5-bromo-N-(2,2-difluoroethyl)-2-nitroaniline (XXXIII) (1.33 g, 4.73 mmol) Fe (3.09 g, 47.33 mmol) and NH₄Cl (3.8 g, 71.05 mmol) dissolved in EtOH (18 mL) and water (6 mL) was heated to 70° C. for 4 h. The reaction mixture was cooled, filtered through Celite®. The filtrates were taken into EtOAc and washed with water, brine, dried over anhydrous Na₂SO₄, filtered, and concentrated under reduced pressure to obtain 4-bromo-2-N-(2,2-difluoroethyl)benzene-1,2-diamine (XXXIV) (1.12 g, 4.461 mmol, 94.3% yield) as a brown oil which was used for the next reaction without further purification. ESIMS found for C₈H₉BrF₂N₂ m/z 251.0 (M+H).

### Step 3

A solution of 4-bromo-2-N-(2,2-difluoroethyl)benzene-1,2-diamine (XXXIV) (1.12 g, 4.46 mmol) and acetic anhydride (0.5 mL, 5.3 mmol) in HOAc (20 mL) was heated to 120° C. for 16 h. The reaction mixture was concentrated, the residue partitioned between EtOAc/1N NaOH, organics separated, washed with water and brine. The organics were dried over anhydrous Na₂SO₄, concentrated, and purified via column chromatography (24 g of silica gel) (0→8% MeOH/CHCl₃) to produce 6-bromo-1-(2,2-difluoroethyl)-2-methylbenzimidazole (XXXV) (970 mg, 3.526 mmol, 79.0% yield) as a beige solid. ESIMS found C₁₀H₉BrF₂N₂ m/z 275.0 (M+H).

Preparation of intermediate 6-bromo-1-(2,2-difluoroethyl)-1H-benzo[d]imidazole (XXXVI) is depicted below in Scheme 7.

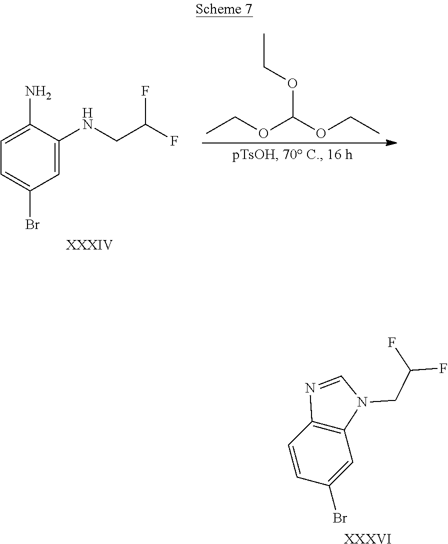

### Step 1

To a solution of 5-bromo-N₁-(2,2-difluoroethyl)benzene-1,2-diamine (XXXIV) (3.0 g, 11.95 mmol) in triethoxymethane (60 mL) at room temperature was added p-toluenesulfonic acid (230 mg, 1.20 mmol). The reaction mixture was stirred at 70° C. for 16 h, then cooled to room temperature. The reaction mixture was concentrated. The reaction mixture was diluted with water (200 mL) and extracted with EtOAc (2×300 mL). The organic layers were concentrated, dried over Na₂SO₄, filtered, and concentrated. The mixture was purified by reverse-phase column chromatography (Regular C18 20-40 um, MeCN/0.1% NH₄HCO₃ aqueous solution, gradient: 40%→60% MeCN) to afford 6-bromo-1-(2,2-difluoroethyl)-1H-benzo[d]imidazole (XXXVI) (1.5 g, 5.746 mmol, 48.1% yield) as a yellow solid. ¹H NMR (400 MHz, DMSO-d₆) δ 8.26 (s 1H), 7.98 (d, J=1.6 Hz, 1H), 7.64 (d, J=8.4 Hz, 1H), 7.38 (dd, J=8.8, 2 Hz, 1H), 6.46 (tt, J=54.3, 3.0 Hz, 1H), 5.36 (td, J=16.4, 3.2 Hz, 2H); ESIMS found for C₉H₇BrF₂N₂ m/z 260.9 (M+H).

Preparation of intermediate 6-bromo-1-(2,2-difluoroethyl)-4-fluoro-1H-benzo[d]imidazole (XXXIX) is depicted below in Scheme 8.

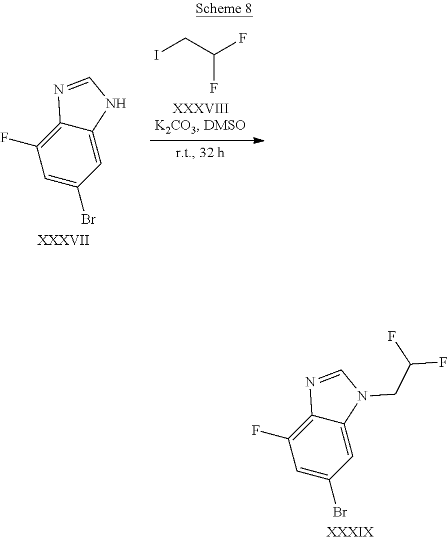

### Step 1

A mixture of K₂CO₃ (1.29 g, 9.3 mmol), 1,1-difluoro-2-iodoethane (XXXVIII) (1.34 g, 7 mmol) and 6-bromo-4-fluoro-1H-1,3-benzodiazole (XXXVII) (commercially available from AA Blocks, LLC) (1.0 g, 4.65 mmol) in DMSO (5 mL) was stirred at room temperature for 16 h. LCMS showed some starting material remained. An additional amount of 1,1-difluoro-2-iodoethane (1.34 g, 7 mmol) was added and stirred at room temperature for an additional 16 h. LCMS showed reaction was complete. The reaction mixture was poured into a solution of EtOAc and water. The layers were separated, and the aqueous phase was washed with EtOAc (3×). All the EtOAc phases were combined, washed with brine solution, dried over MgSO₄, and purified via column chromatography (24 g of silica gel) (50→100% EtOAc/hexanes) to obtain 6-bromo-1-(2,2-difluoroethyl)-4-fluoro-1H-benzo[d]imidazole (XXXIX) (1.14 g, 4.085 mmol, 87.8% yield) as a clear viscous solid that solidified to a white solid upon drying. ESIMS found for C₉H₆BrF₃N₂ m/z 279.0 (M+H).

Preparation of intermediate 6-bromo-1-(2,2-difluoroethyl)-1H-benzo[d][1,2,3]triazole (XL) is depicted below in Scheme 9.

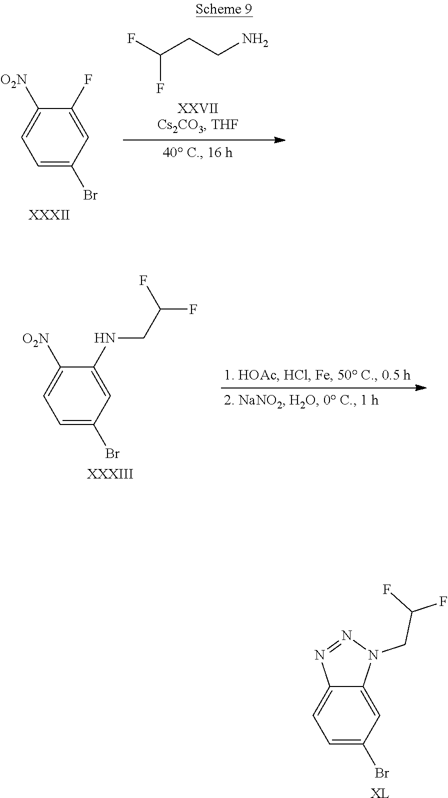

### Step 1

A solution of 4-bromo-2-fluoro-1-nitrobenzene (XXXII) (20.0 g, 98.03 mmol) in THF (500.0 mL) was cooled to 0° C. Cs₂CO₃ (63.9 g, 196.06 mmol) was added, 2,2-difluoroethan-1-amine (XXVII) (36.6 g, 183.81 mmol) was added at 0° C. The reaction was warmed to 40° C. for 16 h. The reaction mixture was extracted with EtOAc (500 L×3). The combined organics were washed with brine (500 mL×3). The combined organic layers were dried with Na₂SO₄, filtered, and concentrated to give the crude product. The crude was purified by column chromatography on silica gel (10→20% EtOAc/PE) to give 5-bromo-N-(2,2-difluoroethyl)-2-nitroaniline (XXXIII) (23 g, 81.83 mmol, 83.5% yield) as a yellow solid. ¹H NMR (400 MHz, DMSO-d₆) δ 3.99 (tdd, J=15.6, 6.6, 3.8 Hz, 2H), 6.29 (tt, J=55.4, 3.7 Hz, 1H), 6.96 (dd, J=9.2, 2.0 Hz, 1H), 7.51 (d, J=1.6 Hz, 1H), 8.05 (d, J=9.2 Hz, 1H), 8.33 (t, J=6.4 Hz, 1H); ESIMS found for C₈H₇BrF₂N₂O₂ m/z 280.9 (M+H).

### Step 2

To a solution of 5-bromo-N-(2,2-difluoroethyl)-2-nitroaniline (XXXIII) (12.0 g, 42.86 mmol) in HOAc/HCl (500 mL/50 mL) was added Fe (30.0 g, 428.62 mmol). The reaction mixture was stirred at 50° C. for 30 minutes, then cooled to room temperature and filtered. NaNO₂ (3.0 g, 53.58 mmol) in water (20 mL) was then added dropwise into the above acid solution at 0° C. The reaction solution was stirred for 1 h at 0° C. The reaction mixture was concentrated to dryness, the reaction mixture was poured into EtOAc (300 mL) and H₂O (300 mL). The pH was adjusted>7 with NaHCO₃. The reaction mixture was extracted with EtOAc (500 mL×3). The combined organics were washed with brine (500 mL×3). The organic layers was concentrated, dried over Na₂SO₄, filtered, and concentrated to give the crude. The crude was purified by column chromatography on silica gel (10→50% EtOAc/PE) to give 6-bromo-1-(2,2-difluoroethyl)-1H-benzo[d][1,2,3]triazole (XL) (5 g, 19.08 mmol, 44.5%) as a brown solid. ¹H NMR (400 MHz, DMSO-d₆) δ 5.41-5.29 (m, 2H), 6.61 (tt, J=54.2, 3.2 Hz, 1H), 7.60 (dd, J=8.8, 1.7 Hz, 1H), 8.08 (d, J=8.8 Hz, 1H), 8.31 (d, J=1.0 Hz, 1H); ESIMS found for CsH₆BrF₂N₃ m/z 261.9 (M+H).

The following intermediates were prepared in accordance with the procedure described in the above Scheme 9.

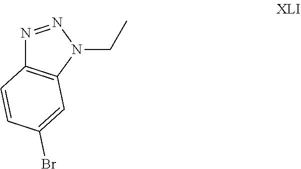

6-Bromo-1-ethyl-1H-benzo[d][1,2,3]triazole (XLI): White solid (4.112 g, 18.188 mmol). ¹H NMR (400 MHz, DMSO-d₆) δ 8.30 (d, J=1.6 Hz, 1H), 8.00 (dd, J=8.8, 3.2 Hz, 1H), 7.54 (dd, J=8.8, 3.2 Hz, 1H), 4.74 (q, J=7.2 Hz, 2H), 1.51 (t, J=7.2 Hz, 3H); ESIMS found for C₈H₈BrN₃ m/z 225.9 (M+H).

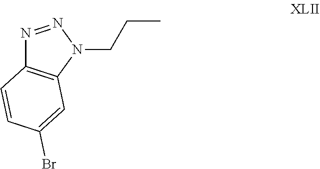

6-Bromo-1-propyl-1H-benzo[d][1,2,3]triazole (XLII): White solid (6.3 g, 26.239 mmol).

¹H NMR (400 MHz, DMSO-d₆) δ ¹H NMR (400 MHz, DMSO-d₆) δ ppm 0.85 (3H, t, J=7.38 Hz), 1.91 (2H, sxt, J=7.23 Hz), 4.66 (2H, t, J=7.00 Hz), 7.52 (1H, dd, J=8.82, 1.56 Hz), 8.00 (1H, d, J=8.75 Hz), 8.29 (1H, d, J=1.50 Hz); ESIMS found for C₉H₁₀BrN₃ m/z 242.0 (⁸¹BrM+H).

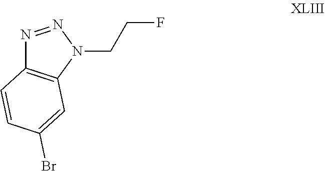 [SMILES: FCCn1nnc2ccc(Br)cc21]

6-Bromo-1-(2-fluoroethyl)-1H-benzo[d][1,2,3]triazole (XLIII): White solid (4.163 g, 17.057 mmol). ¹H NMR (400 MHz, DMSO-d₆) δ 8.29 (s 1H), 8.05 (d, J=8.4 Hz, 1H), 7.57 (dd, J=8.4, 1.6 Hz, 1H), 5.11 (dt, J=28, 4 Hz, 2H), 4.92 (dt, J=48, 4 Hz, 2H); ESIMS found for C₈H₇BrFN₃ m/z 244.0 (M+H).

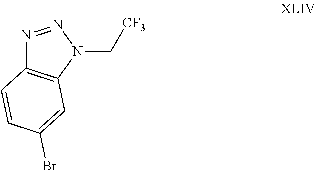 [SMILES: FC(F)(F)Cn1nnc2ccc(Br)cc21]

6-Bromo-1-(2,2,2-trifluoroethyl)-1H-benzo[d][1,2,3]triazole (XLIV): White solid (5.05 g, 18.032 mmol). ¹H NMR (400 MHz, DMSO-d₆) δ 8.38 (s 1H), 8.11 (d, J=8.8 Hz, 1H), 7.63 (dd, J=8.4, 1.6 Hz, 1H), 5.90 (q, J=9.2 Hz, 2H); ESIMS found for C₈H₈BrF₃N₃ m/z 280.0 (M+H).

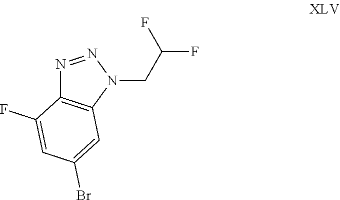 [SMILES: Fc1cc(Br)cc2c1nnn2CC(F)F]

6-Bromo-1-(2,2-difluoroethyl)-4-fluoro-1H-benzo[d][1,2,3]triazole (XLV): White solid (4.112 g, 14.683 mmol). ¹H NMR (400 MHz, DMSO-d₆) δ 8.19 (s 1H), 7.61 (d, J=10.0 Hz, 1H), 6.60 (tt, J=54.3, 3.0 Hz, 1H), 5.36 (td, J=15.81, 2.87 Hz, 2H); ESIMS found for C₈H₅BrF₃N₃ m/z 280.0 (M+H).

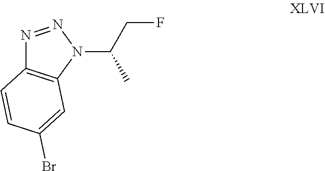

(S)-6-Bromo-1-(1-fluoropropan-2-yl)-1H-benzo[d][1,2,3]triazole (XLVI): White solid (1.06 g, 4.107 mmol). ¹H NMR (400 MHz, DMSO-d₆) δ ¹H NMR (400 MHz, DMSO-d₆) δ ppm 1.65 (3H, dd, J=7.00, 1.38 Hz), 4.87 (2H, dd, J=46.76, 5.63 Hz), 5.39-5.59 (1H, m), 7.56 (1H, dd, J=8.82, 1.69 Hz), 8.04 (1H, d, J=8.75 Hz), 8.31 (1H, d, J=1.50 Hz); ESIMS found for C₉H₉BrFN₃ m/z 257.9 (M+H).

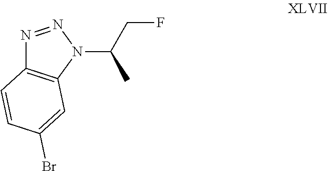

(R)-6-Bromo-1-(1-fluoropropan-2-yl)-1H-benzo[d][1,2,3]triazole (XLVII): White solid (1.036 g, 4.014 mmol).

¹H NMR (400 MHz, DMSO-d₆) δ ¹H NMR (400 MHz, DMSO-d₆) δ ppm 1.65 (3H, dd, J=7.00, 1.38 Hz), 4.86 (2H, dd, J=46.76, 5.75 Hz), 5.39-5.60 (1H, m), 7.56 (1H, dd, J=8.75, 1.75 Hz), 8.04 (1H, d, J=8.75 Hz), 8.31 (1H, d, J=1.38 Hz); ESIMS found for C₉H₉BrFN₃ m/z 257.9 (M+H).

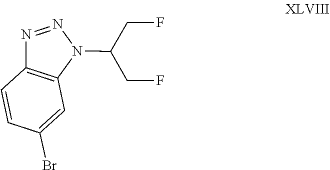 [SMILES: FCC(CF)n1nnc2ccc(Br)cc21]

6-Bromo-1-(1,3-difluoropropan-2-yl)-1H-benzo[d][1,2,3]triazole (XLVIII): White solid (1.064 g, 3.854 mmol). ¹H NMR (400 MHz, DMSO-d₆) δ ¹H NMR (400 MHz, DMSO-d₆) δ ppm 4.97-5.08 (2H, m), 5.09-5.20 (2H, m), 5.70-5.91 (1H, m), 7.59 (1H, dd, J=8.82, 1.69 Hz), 8.08 (1H, d, J=8.75 Hz), 8.35 (1H, d, J=1.50 Hz); ESIMS found for C₉H₈BrF₂N₃ m/z 277.9 (Br⁸¹M+H).

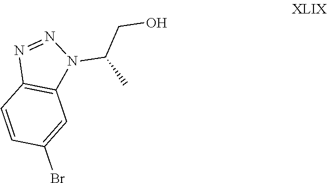

(S)-2-(6-Bromo-1H-benzo[d][1,2,3]triazol-1-yl)propan-1-ol (XLIX): White solid (1.106 g, 4.319 mmol).

¹H NMR (400 MHz, DMSO-d₆) δ ¹H NMR (400 MHz, DMSO-d₆) δ ppm 1.59 (3H, d, J=6.88 Hz), 3.80-3.87 (2H, m), 5.02 (1H, t, J=5.57 Hz), 5.05-5.14 (1H, m), 7.51 (1H, dd, J=8.75, 1.63 Hz), 8.00 (1H, d, J=8.76 Hz), 8.23 (1H, d, J=1.50 Hz); ESIMS found for C₉H₁₀BrN₃O m/z 256.0 (M+H).

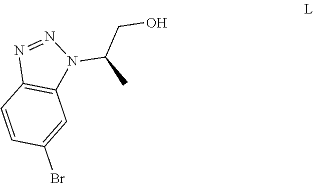

(R)-2-(6-Bromo-1H-benzo[d][1,2,3]triazol-1-yl)propan-1-ol (L): White solid (1.159 g, 4.526 mmol).

¹H NMR (400 MHz, DMSO-d₆) δ ¹H NMR (400 MHz, DMSO-d₆) δ ppm 1.59 (3H, d, J=6.88 Hz), 3.80-3.86 (2H, m), 5.03 (1H, t, J=5.57 Hz), 5.05-5.13 (1H, m), 7.51 (1H, dd, J=8.76, 1.75 Hz), 8.00 (1H, d, J=8.76 Hz), 8.23 (1H, d, J=1.25 Hz); ESIMS found for C₉H₁₀BrN₃O m/z 256.0 (M+H).

Preparation of intermediate 1-(2-fluoroethyl)-6-(4,4,5,5-tetramethyl-1,3,2-dioxaborolan-2-yl)-1H-benzo[d][1,2,3]triazole (LI) is depicted below in Scheme 10.

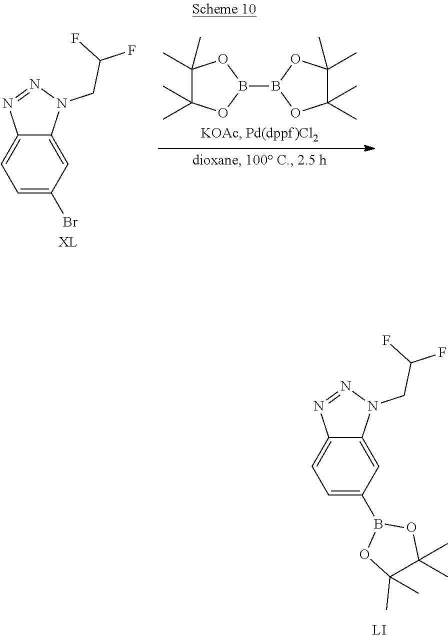

### Step 1

6-Bromo-1-(2,2-difluoroethyl)-1H-benzo[d][1,2,3]triazole (XL) (5.16 g, 19.69 mmol), bis(pinacolato)diboron (7.51 g, 29.57 mmol), Pd(dppf)Cl₂ (803 mg, 0.98 mmol) and KOAc (5.8 g, 59.1 mmol) were suspended in dry 1,4-dioxane (100 mL). The reaction was sonicated and degassed for 5 minutes before heating at 100° C. for 2.5 h. The reaction mixture was loaded onto Celite® and purified by column chromatography (0→50% EtOAc/hexanes) to produce 1-(2,2-difluoroethyl)-6-(4,4,5,5-tetramethyl-1,3,2-dioxaborolan-2-yl)-1H-benzo[d][1,2,3]triazole (LI) (5.654 g, 18.291 mmol, 92.9% yield) as a fluffy pale-orange solid. ESIMS found for C₁₄H₁₈BF₂N₃O₂ m/z 310.15 (M+H).

The following intermediates were prepared in accordance with the procedure described in the above Scheme 10.

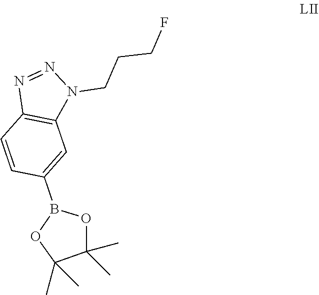 [SMILES: CC1(C)OB(c2ccc3nnn(CCCF)c3c2)OC1(C)C]

1-(3-Fluoropropyl)-6-(4,4,5,5-tetramethyl-1,3,2-dioxaborolan-2-yl)-1H-benzo[d][1,2,3]triazole (LII): White solid (2.712 g, 8.887 mmol).

¹H NMR (400 MHz, DMSO-d₆) δ ¹H NMR (400 MHz, DMSO-d₆) δ ppm 1.34 (12H, s), 2.21-2.44 (2H, m), 4.49 (2H, dt, J=47.24, 5.60 Hz), 4.85 (2H, dt, J=25.28, 7.00 Hz), 7.65 (1H, dd, J=8.32, 0.69 Hz), 8.03 (1H, dd, J=8.44, 0.81 Hz), 8.19 (1H, s); ESIMS found for C₁₅H₂₁BFN₃O₂ m/z 306.1 (M+H).

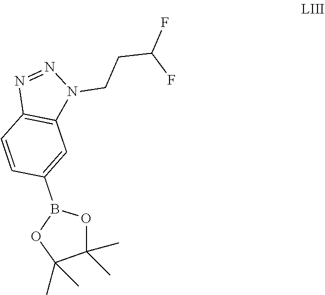 [SMILES: CC1(C)OB(c2ccc3nnn(CCC(F)F)c3c2)OC1(C)C]

1-(3,3-Difluoropropyl)-6-(4,4,5,5-tetramethyl-1,3,2-dioxaborolan-2-yl)-1H-benzo[d][1,2,3]triazole (LIII): White solid (5.0 g, 15.473 mmol).

¹H NMR (400 MHz, DMSO-d₆) δ ¹H NMR (400 MHz, DMSO-d₆) δ ppm 1.34 (12H, s), 2.51-2.62 (2H, m), 4.95 (2H, t, J=6.94 Hz), 6.22 (1H, tt, J=56.00, 4.31 Hz), 7.66 (1H, d, J=8.38 Hz), 8.03 (1H, d, J=8.38 Hz), 8.21 (1H, s); ESIMS found for C₁₅H₂₀BF₂N₃O₂ m/z 324.2 (M+H).

Preparation of intermediate 6-bromo-N-methylimidazo[1,2-a]pyridine-3-carboxamide (LV) is depicted below in Scheme 11.

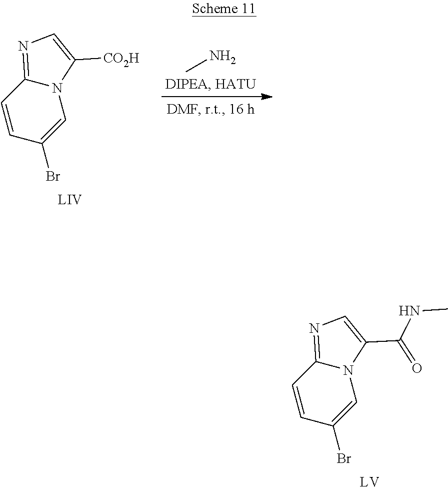

### Step 1

To a solution of 6-bromoimidazo[1,2-a]pyridine-3-carboxylic acid (LIV) (commercially available from Advanced ChemBlocks Inc.) (1 g, 4.15 mmol) in DMF (15 mL) was added HATU (1.9 g, 5 mmol) at room temperature and stirred for 15 min. The solution was cooled to −60° C. before adding methanamine-HCl (2.8 g, 41.47 mmol) followed by DIPEA (8.7 mL, 49.95 mmol). The mixture was stirred at room temperature for 16 h. The reaction mixture was quenched with a saturated aqueous NaHCO₃ solution, concentrated, absorbed on silica gel, and purified by ISCO (0→15% MeOH in CHCl₃). The appropriate fractions were collected, concentrated, and dried on high vacuo to obtain 6-bromo-N-methylimidazo[1,2-a]pyridine-3-carboxamide (LV) (460 mg, 1.811 mmol, 43.6% yield) as an off-white solid. ESIMS found for C₉H₈BrN₃O m/z 254.0 (M+H).

The following intermediates were prepared in accordance with the procedure described in the above Scheme 11.

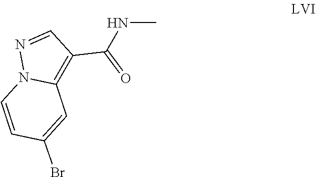

5-Bromo-N-methylpyrazolo[1,5-a]pyridine-3-carboxamide (LVI): Off-white solid (290 mg, 1.141 mmol, 91.7% yield). ESIMS found for C₉H₈BrN₃O m/z 253.95 (M+H).

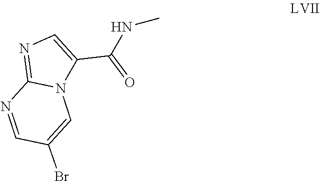

6-Bromo-N-methylimidazo[1,2-a]pyrimidine-3-carboxamide (LVII): Off-white solid (576 mg, 2.258 mmol, 91.1% yield). ESIMS found for C₈H₇BrN₄O m/z 255.0 (M+H).

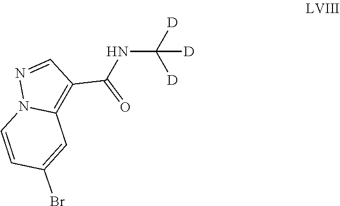 [SMILES: [2H]C([2H])([2H])NC(=O)c1cnn2ccc(Br)cc12]

5-Bromo-N-(methyl-d₃)pyrazolo[1,5-a]pyridine-3-carboxamide (LVIII): Off-white solid (500 mg, 1.945 mmol, 93.8% yield). ESIMS found for C₉H₅D₃BrN₃O m/z 257.0 (M+H).

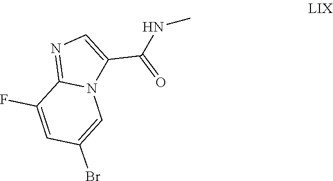

6-Bromo-8-fluoro-N-methylimidazo[1,2-a]pyridine-3-carboxamide (LIX): Beige solid (320 mg, 1.176 mmol, 50.8% yield). ESIMS found for C₉H₇BrFN₃O m/z 272.0 (M+H).

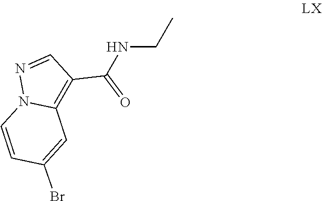

5-Bromo-N-ethylpyrazolo[1,5-a]pyridine-3-carboxamide (LX): Off-white solid (500 mg, 1.865 mmol, 89.9% Yield). ESIMS found for C₁₀H₁₀BrN₃O m/z 268.0 (M+H).

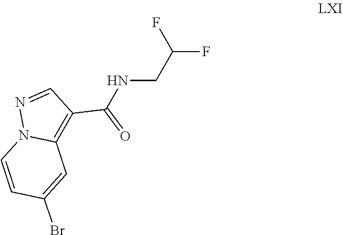 [SMILES: O=C(NCC(F)F)c1cnn2ccc(Br)cc12]

5-Bromo-N-(2,2-difluoroethyl)pyrazolo[1,5-a]pyridine-3-carboxamide (LXI): White solid (576 mg, 1.894 mmol, 91.3% yield). ESIMS found for C₁₀H₈BrF₂N₃O m/z 304.0 (M+H).

Preparation of intermediate 6-bromo-3-(difluoromethyl)-8-fluoroimidazo[1,2-a]pyridine (LXV) is depicted below in Scheme 12.

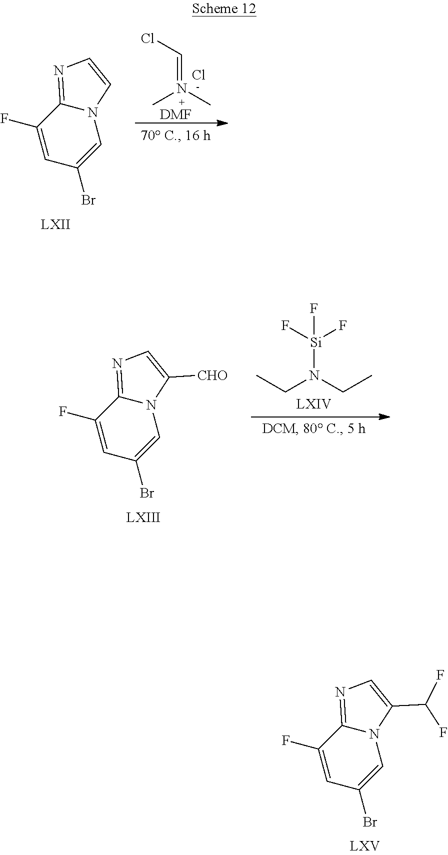

### Step 1

To a stirred suspension of the Vilsmeier reagent (720 mg, 5.59 mmol) in DMF (2 mL) was added 6-bromo-8-fluoroimidazo[1,2-a]pyridine (LXII) (400 mg, 1.86 mmol) in DMF (4 mL). The mixture was heated to 70° C. for 16 h. The reaction mixture was cooled, added to ice, basified with 2 N NaOH, stirred for 1 h and the solids were collected by filtration. The solids were taken in CHCl₃, washed with brine solution, dried over anhydrous Na₂SO₄, evaporated, and dried under high vacuo to obtain 6-bromo-8-fluoroimidazo[1,2-a]pyridine-3-carbaldehyde (LXIII) (360.0 mg, 1.481 mmol, 79.6% yield) as a beige solid. ESIMS found for C₈H₄BrFN₂O m/z 243.1 (M+H).

### Step 2

To a stirred solution of 6-bromo-8-fluoroimidazo[1,2-a]pyridine-3-carbaldehyde (LXIII) (500 mg, 2.06 mmol) in DCM (6 mL) in a plastic seal tube was added drop wise (diethylamino)sulfur trifluoride (LXIV) (1.5 mL, 12.13 mmol) at room temperature. The reaction mixture was heated to 80° C. for 5 h. The reaction mixture was cooled to room temperature, diluted with DCM, washed with aqueous saturated NaHCO₃, brine, dried over anhydrous Na₂SO₄, and concentrated. The residue was purified by ISCO (0→60% EtOAc/hexanes) to obtain 6-bromo-3-(difluoromethyl)-8-fluoroimidazo[1,2-a]pyridine (LXV) (437 mg, 1.649 mmol, 80.1% yield) as an off-white solid. ESIMS found for C₈H₄BrF₃N₂ m/z 264.95 (M+H).

Preparation of intermediate imidazo[1,2-a]pyrimidin-6-ylboronic acid (LXVIII) is depicted below in Scheme 13.

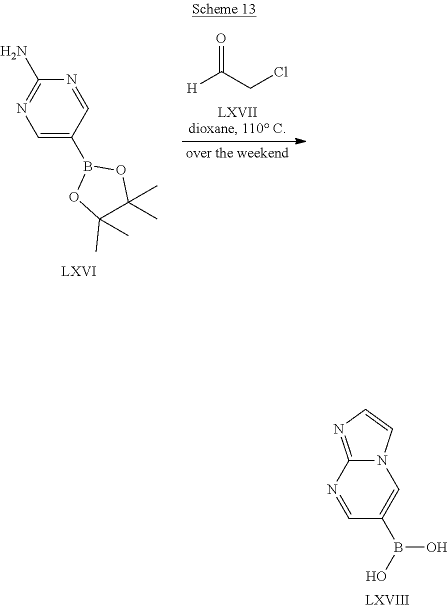

### Step 1

A mixture of 5-(4,4,5,5-tetramethyl-1,3,2-dioxaborolan-2-yl)pyrimidin-2-amine (LXVI) (1 g, 4.52 mmol) and chloroacetaldehyde (LXVII) (0.92 mL, 5.39 mmol) was dissolved in 1,4-dioxane (20 mL) and heated to 110° C. over the weekend. The reaction mixture was cooled, and the solids were collected by filtration and dried under high vacuo to obtain imidazo[1,2-a]pyrimidin-6-ylboronic acid (LXVIII) (650 mg, 3.989 mmol, 88.2% yield) as a brown solid which was used for next step without purification. ESIMS found for C₆H₆BN₃O₂ m/z 164.1 (M+H).

Preparation of intermediate N-(trans-4-aminocyclohexyl)acetamide (LXXI) is depicted below in Scheme 14.

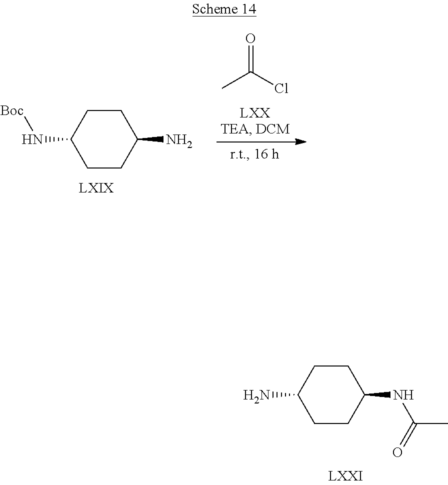

### Step 1

To a stirring solution of tert-butyl N-(4-aminocyclohexyl)carbamate (LXIX) (commercially available from Combi-Blocks Inc.) (0.6 g, 2.8 mmol) in DCM (6 mL) was added TEA (1.2 mL, 8.61 mmol). Acetyl chloride (LXX) (0.22 mL, 3.09 mmol) was then slowly and the reaction mixture was stirred at room temperature for 16 h. The solvent was removed, and the crude material was dissolved in EtOAc, washed with 1 M NaOH, brine, and dried over anhydrous MgSO₄ and finally concentrated. The product was dissolved in EtOH (2 mL) and 4 M HCl (1 mL). The solution was stirred at room temperature for 2 h before evaporating to dryness to give the HCl salt of N-(4-aminocyclohexyl)acetamide (LXXI) (480 mg, 2.49 mmol, 89.0% yield) as a white solid. ESIMS found for C₈H₁₆N₂O m/z 157.05 (M+H).

Preparation of intermediate cis-3-amino-1-ethylcyclobutan-1-ol (LXXX) is depicted below in Scheme 15.

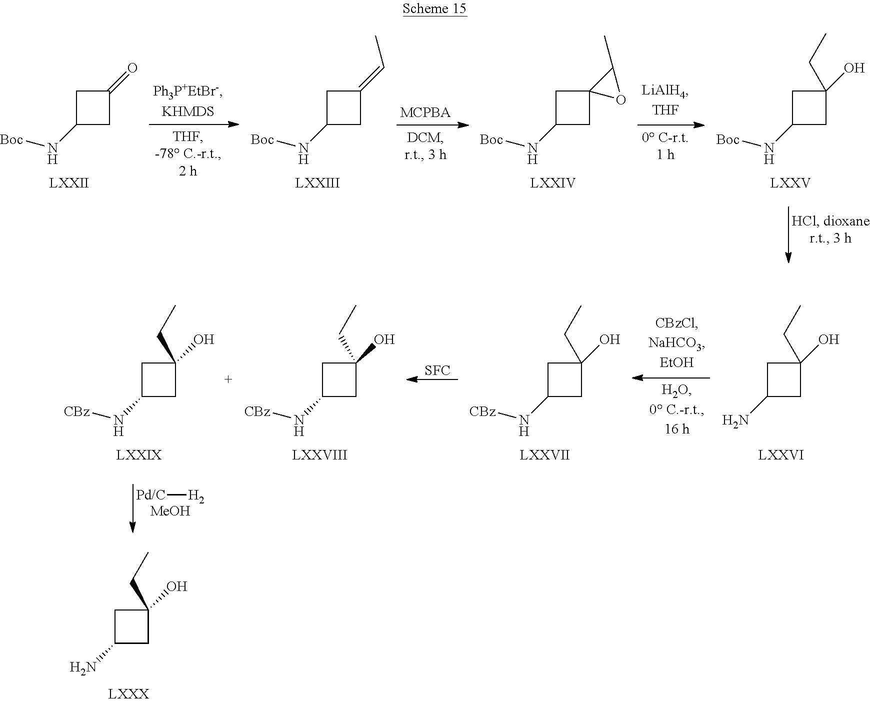

### Step 1

To a cloudy solution of ethyltriphenylphosphonium bromide (30.08 g, 81.08 mmol) in THF (240 mL) at −78° C. under N₂ was added KHMDS (80 mL, 81.08 mmol) at −78° C. The mixture was stirred for 30 minutes before adding tert-butyl (3-oxocyclobutyl)carbamate (LXXII) (10 g, 54.05 mmol) in THF (50 mL) dropwise at −78° C. The reaction mixture was stirred at room temperature for 2 h. The reaction solution was quenched with brine (200 mL) and extracted with EtOAc (2×200 mL). The organic layer was washed with H₂O (150 mL) and brine (150 mL), then separated, dried over anhydrous Na₂SO₄, and concentrated in vacuo. The crude was purified by silica gel column chromatography (silica, 100-200 mesh, 10% EtOAc/PE) to obtain tert-butyl (3-ethylidenecyclobutyl)carbamate (LXXIII) (1.5 g, 7.603 mmol, 14.1% yield) as off-white solid. H NMR (400 MHz, CDCl₃) δ ppm 1.37 (s, 9H), 1.43 (dd, J=7.0, 3.4 Hz, 3H), 2.49-2.29 (m, 2H), 2.93 (s, 2H), 4.05 (s, 1H), 4.69 (s, 1H), 5.15 (s, 1H).

### Step 2

To a solution of tert-butyl (3-ethylidenecyclobutyl)carbamate (LXXIII) (1.0 g, 5.08 mmol) in DCM (40 mL) at 0° C. was added 3-chloro-benzenecarboperoxoic acid (1.75 g, 10.15 mmol). The mixture was stirred at room temperature for 3 h. The mixture was quenched with brine (50 mL) and extracted with DCM (2×80 mL). The organic layer was separated, then washed with saturated aqueous NaHCO₃ solution (50 mL) and brine (50 mL). The organic layer was separated, dried over anhydrous Na₂SO₄, and concentrated in vacuo to give tert-butyl (2-methyl-1-oxaspiro[2.3]hexan-5-yl)carbamate (LXXIV) (1.0 g, 4.689 mmol, 92.3% yield) as a white solid which was used in the next step without further purification. ESIMS found for C₁₁H₁₉NO₃ m/z 158.1 (M-^{t}Bu+H).

### Step 3

To a solution of tert-butyl (2-methyl-1-oxaspiro[2.3]hexan-5-yl)carbamate (LXXIV) (1.0 g, 4.69 mmol) in THF (10 mL) at 0° C. was added LiAlH4 (6 mL, 6.17 mmol). The mixture was stirred at room temperature for 1 h. The mixture was added sodium sulfate decahydrate and then filtered through a Celite® pad, and the filtrate was concentrated to give the crude product. The crude product was chromatographed on silica gel (silica, 100-200 mesh, 30% EtOAc/PE) to give the tert-butyl (3-ethyl-3-hydroxycyclobutyl)carbamate (LXXV) (200 mg, 0.929 mmol, 19.8% yield) as a white oil. ¹H NMR (400 MHz, DMSO-d₆) δ ppm 0.79 (dt, J=19.6, 7.4 Hz, 3H), 1.36 (s, 9H), 1.43 (dd, J=14.4, 7.2 Hz, 2H), 1.81 (dd, J=14.2, 5.6 Hz, 2H), 2.26-2.02 (m, 2H), 4.05-3.37 (m, 1H), 4.60 (d, J=45.8 Hz, 1H), 6.90 (t, J=73.8 Hz, 1H).

### Step 4

tert-Butyl (3-ethyl-3-hydroxycyclobutyl)carbamate (LXXV) (500 mg, 2.33 mmol) was dissolved in a solution of 4 M HCl in dioxane (5 mL). The mixture was stirred at room temperature for 1 h. The mixture was concentrated and dried under vacuum to produce desired 3-amino-1-ethylcyclobutan-1-ol (LXXVI) (500 mg, assuming quantitative) as a yellow oil which was used in the next step without further purification.

### Step 5

To a solution of crude 3-amino-1-ethylcyclobutan-1-ol (LXXVI) (500 mg, 2.33 mmol) in mixture of EtOAc (20 mL) and aqueous NaHCO₃ (10 mL) at 0° C. was added benzyl chloroformate (515.7 mg, 3.02 mmol). The mixture was stirred at room temperature for 16 h. The mixture was quenched with brine (50 mL) and extracted with EtOAc (2×100 mL). The organic layer was separated, dried over anhydrous Na₂SO₄, and concentrated in vacuo. The crude product was chromatographed on silica gel (silica, 100-200 mesh, 30% EtOAc/PE) to yield the racemic benzyl (3-ethyl-3-hydroxycyclobutyl)carbamate (LXXVII) (450 mg, 1.805 mmol, 77.5% yield) as a white solid. ¹H NMR (400 MHz, DMSO-d₆) δ ppm 0.80 (dt, J=19.6, 7.4 Hz, 3H), 1.44 (dt, J=13.8, 7.0 Hz, 2H), 1.90-1.77 (m, 2H), 2.30-2.06 (m, 2H), 4.20-3.39 (m, 1H), 4.66 (d, J=47.4 Hz, 1H), 4.99 (s, 2H), 7.54-7.09 (m, 6H); ESIMS found for C₁₄H₁₉NO₃ m/z 250.1 (M+H).

Racemic benzyl (3-ethyl-3-hydroxycyclobutyl)carbamate (LXXVII) (2.5 g, 10.0 mmol) was purified by SFC (Table 2) to afford the benzyl (cis-3-ethyl-3-hydroxycyclobutyl)carbamate (LXXIX) (1.8 g, 7.220 mmol) as a white solid ¹H NMR (400 MHz, DMSO-d₆) δ ppm 0.82 (t, J=7.2 Hz, 3H), 1.43 (q, J=7.2 Hz, 2H), 1.85 (td, J=8.8, 2.4 Hz, 2H), 2.33-2.14 (m, 2H), 3.59-3.40 (m, 1H), 4.72 (s, 1H), 4.99 (s, 2H), 7.60-7.09 (m, 6H), ESIMS found for C₁₄H₁₉NO₃ m/z 250.1 (M+H), and benzyl (trans-3-ethyl-3-hydroxycyclobutyl)carbamate (LXXVIII) (0.3 g, 1.203 mmol) as a white solid.

TABLE 2
Chiral Pre-SFC Method

| | |
|---|---|
| System: | Waters SFC 150 |
| Column name: | CHIRALPAK ® IG (Daicel Corporation) |
| Column size: | 250*25 mm 10 μm |
| Mobile Phase A: | Supercritical CO₂, |
| Mobile Phase B: | MEOH (+0.1% 7.0 mol/L Ammonia in MEOH) |
| A:B: | 80:20 |
| Wavelength: | 214 nm |
| Flow: | 120 ml/min |
| Column temp: | RT |
| Back Pressure: | 100 bar |
| Injection: | 2 mL |
| Cycle time: | 7.04 min |
| Solvent: | MEOH: redistilled grade |
| | Supercritical CO₂: Food grade |
| Preparation of | sample was dissolved in about 95 mL MeOH |
| sample solution: | |

### Step 6

To a solution of benzyl (cis-3-ethyl-3-hydroxycyclobutyl)carbamate (LXXIX) (1.8 g, 7.22 mmol) in MeOH (50 mL) was added Pd/C (360 mg). The mixture was stirred at room temperature under H₂ (1 atm) for 16 h. The mixture was filtered through Celite®, and the filtrate was concentrated to give cis-3-amino-1-ethylcyclobutan-1-ol (LXXX) (540 mg, 4.688 mol, 64.9% yield) as a yellow solid. ¹H NMR (400 MHz, DMSO-d₆) δ ppm 0.80 (t, J=7.4 Hz, 3H), 1.37 (q, J=7.4 Hz, 2H), 1.64-1.54 (m, 2H), 2.27-2.17 (m, 2H), 2.85-2.73 (m, 1H), 3.17 (s, 1H).

The following intermediate was prepared in accordance with the procedure described in the above Scheme 15.

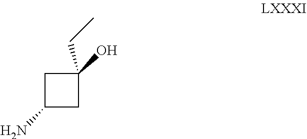

trans-3-Amino-1-ethylcyclobutan-1-ol (LXXXI): ¹H NMR (400 MHz, DMSO-d₆) δ ppm 0.77 (t, J=7.4 Hz, 3H), 1.46 (q, J=7.4 Hz, 2H), 1.58-1.50 (m, 2H), 2.12-2.03 (m, 2H), 3.17 (s, 1H), 3.52-3.38 (m, 1H).

### Example 1

Preparation of (R)—N-(3,3-difluoropiperidin-4-yl)-5-(8-fluoroquinolin-6-yl)-4-methoxypyrrolo[2,1-f][1,2,4]triazin-2-amine (1693), (R)—N-(3,3-difluoro-1-methylpiperidin-4-yl)-5-(8-fluoroquinolin-6-yl)-4-methoxypyrrolo[2,1-f][1,2,4]triazin-2-amine (1694), (R)-1-(3,3-difluoro-4-((5-(8-fluoroquinolin-6-yl)-4-methoxypyrrolo[2,1-f][1,2,4]triazin-2-yl)amino)piperidin-1-yl)ethan-1-one (1695), and (R)—N-(3,3-difluoro-1-(oxetan-3-yl)piperidin-4-yl)-5-(8-fluoroquinolin-6-yl)-4-methoxypyrrolo[2,1-f][1,2,4]triazin-2-amine (1696) are depicted below in Scheme 16.

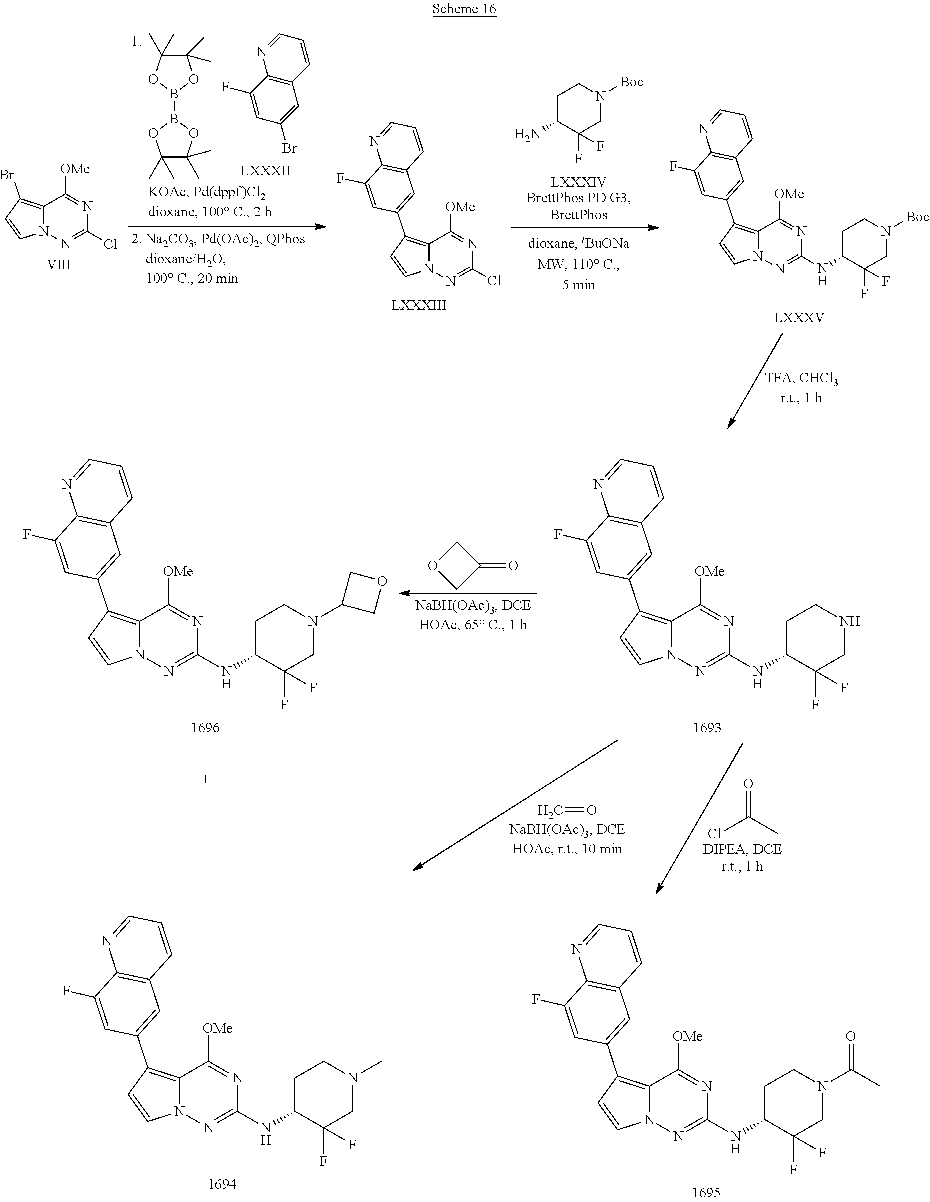

### Steps 1-2

6-Bromo-8-fluoroquinoline (LXXXII) (300 mg, 1.33 mmol), bis(pinacolato)diboron (404 mg, 1.59 mmol), Pd(dppf)Cl₂ (45 mg, 0.06 mmol), KOAc (334 mg, 3.4 mmol) were suspended in dry 1,4-dioxane (7 mL). The reaction was sonicated and was purged with N₂ for 5 min before heating at 100° C. for 2 h. The LC/MS shows a mixture of the boronic acid and boronic ester.

The reaction mixture was cooled and 1 M aqueous solution of Na₂CO₃ (3.5 mL, 3.5 mmol), 5-bromo-2-chloro-4-methoxypyrrolo[2,1-f][1,2,4]triazine (VIII) (300 mg, 1.14 mmol), Pd(OAc)₂ (13 mg, 0.06 mmol) and QPhos (81 mg, 0.11 mmol) were added. The reaction was purged with Ar for 5 min. The reaction was then heated to 100° C. for 20 min. The reaction mixture was added to aqueous saturated NH₄Cl solution before adding CHCl₃. The organic layer was separated, and the aqueous layer was extracted with CHCl₃ (×3). The combined organic layers were stripped onto Celite® and purified by column chromatography (0→100% EtOAc/hexanes) to give 6-(2-chloro-4-methoxypyrrolo[2,1-f][1,2,4]triazin-5-yl)-8-fluoroquinoline (LXXXIII) (366 mg, 1.113 mmol, 97.4% yield) as a beige solid. ESIMS found for C₁₆H₁₀ClFN₄O m/z 329.1 (M+H).

### Step 3

6-(2-Chloro-4-methoxypyrrolo[2,1-f][1,2,4]triazin-5-yl)-8-fluoroquinoline (LXXXIII) (366 mg, 1.11 mmol), tert-butyl (R)-4-amino-3,3-difluoropiperidine-1-carboxylate (LXXXIV) (264 mg, 1.12 mmol), BrettPhos PD G3 (50 mg, 0.06 mmol), BrettPhos (59 mg, 0.11 mmol) and sodium tert-butoxy (267 mg, 2.78 mmol) were added to dry 1,4-dioxane (6 mL) in a microwave vial. The suspension was purged with N₂ then 3 Å MS (5 beads) were added and stirred at room temperature for 30 min. The beads were removed and then the reaction was irradiated to 110° C. for 5 min. The reaction mixture was added to EtOAc, and water and the organic layer was separated. The aqueous layer was extracted with EtOAc (×3) and the combined organic layers stripped onto Celite® and purified by silica gel column chromatography (0→50% EtOAc/hexanes) to produce tert-butyl (R)-3,3-difluoro-4-((5-(8-fluoroquinolin-6-yl)-4-methoxypyrrolo[2,1-f][1,2,4]triazin-2-yl)amino) piperidine-1-carboxylate (LXXXV) (177 mg, 0.335 mmol, 30.1% yield) as a white solid. ESIMS found for C₂₆H₂₇F₃N₆O₃ m/z 529.2 (M+H).

### Step 4

tert-Butyl (R)-3,3-difluoro-4-((5-(8-fluoroquinolin-6-yl)-4-methoxypyrrolo [2,1-f][1,2,4]triazin-2-yl)amino)piperidine-1-carboxylate (LXXXV) (177 mg, 0.335 mmol) was added to TFA (2 mL) in CHCl₃ (4 mL) and stirred at room temperature for 1 h. The solvent was removed under vacuum and the residue was purified by silica gel column chromatography (0→10% 7 NH₃ in MeOH/CHCl₃) to produce (R)—N-(3,3-difluoropiperidin-4-yl)-5-(8-fluoroquinolin-6-yl)-4-methoxypyrrolo[2,1-f][1,2,4]triazin-2-amine (1693) (141 mg, 0.329 mmol, 98.3% yield) as a white solid. ¹H NMR (500 MHz, DMSO-d₆) δ ppm 1.71 (1H, qd, J=12.12, 3.70 Hz), 1.81-1.93 (1H, m), 2.62 (1H, br t, J=11.53 Hz), 2.84 (1H, dd, J=29.40, 13.72 Hz), 2.94 (1H, br d, J=11.80 Hz), 3.09-3.21 (1H, m), 4.02 (3H, s), 4.28-4.44 (1H, m), 6.85 (1H, d, J=2.47 Hz), 6.91 (1H, d, J=9.33 Hz), 7.63 (1H, dd, J=8.51, 4.12 Hz), 7.67 (1H, d, J=2.74 Hz), 7.85 (1H, dd, J=12.62, 1.65 Hz), 7.99 (1H, d, J=1.65 Hz), 8.44 (1H, d, J=8.51 Hz), 8.91 (1H, dd, J=4.12, 1.65 Hz); ESIMS found for C₂₁H₁₉F₃N₆O m/z 429.15 (M+1).

### Step 5a

To a suspension of oxetan-3-one (17.9 μL, 0.31 mmol) in dry DCE (0.96 mL) was added HOAc (18 μL, 0.31 mmol) and (R)—N-(3,3-difluoropiperidin-4-yl)-5-(8-fluoroquinolin-6-yl)-4-methoxypyrrolo[2,1-f][1,2,4]triazin-2-amine (1693) (45 mg, 0.11 mmol). 3 Å MS (5 beads) were added and the reaction was heated to 65° C. for 1 h. NaBH(OAc)₃ (45 mg, 0.21 mmol) was added, and the reaction was stirred for 10 min. The reaction was incomplete so an addition portion of NaBH(OAc)₃ (45 mg, 0.21 mmol), HOAc (18 μL, 0.31 mmol), oxetan-3-one (17.9 μL, 0.31 mmol) was added and the reaction was heated to 65° C. for another 1 h. LCMS confirmed the reaction to be complete. CHCl₃ (10 mL) was added, and the reaction was washed with saturated aqueous NaHCO₃ (10 mL). The CHCl₃ layer was stripped onto Celite® and purified by column chromatography (0→5% MeOH/CHCl3) to give (R)—N-(3,3-difluoro-1-(oxetan-3-yl)piperidin-4-yl)-5-(8-fluoroquinolin-6-yl)-4-methoxypyrrolo[2,1-f][1,2,4]triazin-2-amine (1696) (33 mg, 0.068 mmol, 64.8% yield) as a white solid. ¹H NMR (500 MHz, DMSO-d₆) δ ppm 1.77-1.87 (1H, m), 1.87-1.93 (1H, m), 2.16 (1H, br t, J=10.57 Hz), 2.41 (1H, dd, J=27.00, 11.53 Hz), 2.76 (1H, br d, J=11.53 Hz), 2.96-3.08 (1H, m), 3.60 (1H, quin, J=6.17 Hz), 4.02 (3H, s), 4.23-4.38 (1H, m), 4.44 (2H, dt, J=15.09, 6.17 Hz), 4.55 (2H, td, J=6.52, 3.71 Hz), 6.86 (1H, d, J=2.47 Hz), 6.94 (1H, d, J=9.33 Hz), 7.63 (1H, dd, J=8.23, 4.12 Hz), 7.67 (1H, d, J=2.47 Hz), 7.85 (1H, dd, J=12.49, 1.51 Hz), 7.99 (1H, d, J=1.37 Hz), 8.44 (1H, d, J=8.51 Hz), 8.91 (1H, dd, J=4.12, 1.37 Hz); ESIMS found for C₂₄H₂₃F₃N₆O₂ m/z 485.2 (M+1).

### Step 5b

To a suspension of (R)—N-(3,3-difluoropiperidin-4-yl)-5-(8-fluoroquinolin-6-yl)-4-methoxypyrrolo[2,1-f][1,2,4]triazin-2-amine (1693) (45 mg, 0.11 mmol) in dry DCE (0.96 mL) was added HOAc (18 μL, 0.31 mmol) and formaldehyde (9 μL, 0.33 mmol) and the reaction was stirred at room temperature for 30 min. NaBH(OAc)₃ (44 mg, 0.21 mmol) was added, and the reaction was stirred at room temperature for another 10 min. CHCl₃ (10 mL) was added, and the reaction was washed with saturated aqueous NaHCO₃ (10 mL). The CHCl₃ layer was stripped onto Celite® and purified by column chromatography (0→5% 7 N NH₃ in MeOH/CHCl3) to yield (R)—N-(3,3-difluoro-1-methylpiperidin-4-yl)-5-(8-fluoroquinolin-6-yl)-4-methoxypyrrolo[2,1-f][1,2,4]triazin-2-amine (1694) (20 mg, 0.045 mmol, 43.0% yield) as a white solid. ¹H NMR (500 MHz, DMSO-d₆) δ ppm 1.77-1.90 (2H, m), 2.18 (1H, td, J=11.05, 3.16 Hz), 2.26 (3H, s), 2.38 (1H, dd, J=27.70, 11.80 Hz), 2.79 (1H, br d, J=11.53 Hz), 2.98-3.13 (1H, m), 4.02 (3H, s), 4.17-4.32 (1H, m), 6.86 (1H, d, J=2.74 Hz), 6.88 (1H, d, J=9.61 Hz), 7.63 (1H, dd, J=8.37, 4.25 Hz), 7.67 (1H, d, J=2.74 Hz), 7.85 (1H, dd, J=12.49, 1.78 Hz), 7.99 (1H, d, J=1.65 Hz), 8.44 (1H, d, J=8.23 Hz), 8.91 (1H, dd, J=4.12, 1.65 Hz); ESIMS found for C₂₂H₂₁F₃N₆O m/z 443.2 (M+1).

### Step 5c

To a reaction flask was added (R)—N-(3,3-difluoropiperidin-4-yl)-5-(8-fluoroquinolin-6-yl)-4-methoxypyrrolo[2,1-f][1,2,4]triazin-2-amine (1693) (45 mg, 0.11 mmol) followed by acetyl chloride (12 μL, 0.17 mmol) and DIPEA (54 μL, 0.31 mmol). This solution was stirred at room temperature for 1 h. The reaction was stripped onto Celite® and purified by silica gel column chromatography (0→100% EtOAc/hexanes) to produce (R)-1-(3,3-difluoro-4-((5-(8-fluoroquinolin-6-yl)-4-methoxypyrrolo[2,1-f][1,2,4]triazin-2-yl)amino)piperidin-1-yl)ethan-1-one (1695) (23 mg, 0.049 mmol, 46.5% yield) as an off-white solid. ¹H NMR (500 MHz, DMSO-d₆) δ ppm 1.58-1.85 (1H, m), 1.85-1.98 (1H, m), 2.01-2.09 (3H, m), 2.93-3.05 (1H, m), 3.61-3.93 (1H, m), 4.03 (3H, s), 4.10-4.34 (1H, m), 4.44-4.62 (2H, m), 6.87 (1H, d, J=2.74 Hz), 7.04 (1H, d, J=9.33 Hz), 7.63 (1H, dd, J=8.23, 4.12 Hz), 7.67 (1H, dd, J=2.47, 1.37 Hz), 7.85 (1H, dd, J=12.62, 1.65 Hz), 7.99 (1H, d, J=1.65 Hz), 8.44 (1H, d, J=8.51 Hz), 8.91 (1H, dd, J=4.12, 1.37 Hz); ESIMS found for C₂₃H₂₁F₃N₆O₂ m/z 471.1 (M+1).

### Example 2

Preparation of N-((1r,3r)-3-((5-(imidazo[1,2-a]pyrimidin-6-yl)-4-methoxypyrrolo[2,1-f][1,2,4]triazin-2-yl)amino)-1-methylcyclobutyl)cyclopropanecarboxamide (1634) are depicted below in Scheme 17.

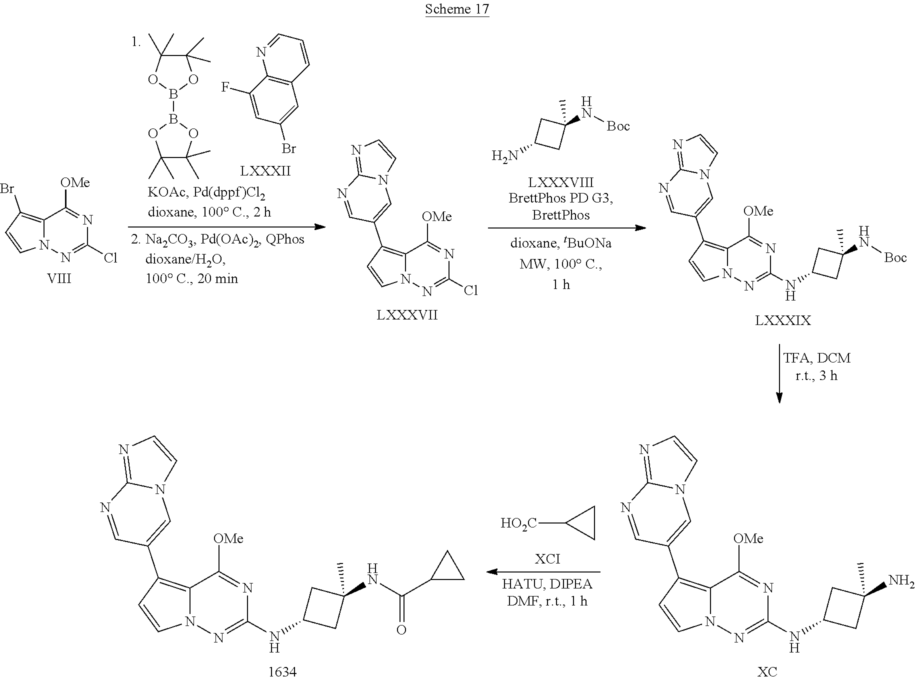

### Steps 1-2

Used procedure described in Example 1, Scheme 16, steps 1-2 to produce 2-chloro-5-(imidazo[1,2-a]pyrimidin-6-yl)-4-methoxypyrrolo[2,1-f][1,2,4]triazine (LXXXVII) (1.92 g, 6.385 mmol). ¹H NMR (400 MHz, DMSO-d₆) δ ppm 4.08 (3H, s), 7.18 (1H, d, J=2.75 Hz), 7.76 (1H, d, J=1.13 Hz), 7.97 (1H, d, J=1.13 Hz), 8.15 (1H, d, J=2.75 Hz), 8.78 (1H, d, J=2.50 Hz), 9.18 (1H, d, J=2.38 Hz); ESIMS found for C₁₃H₉ClN₆O m/z 301.1 (M+H).

### Step 3

Used procedure described in Example 1, Scheme 16, step 3 to produce tert-butyl ((1r,3r)-3-((5-(imidazo[1,2-a]pyrimidin-6-yl)-4-methoxypyrrolo[2,1-f][1,2,4]triazin-2-yl)amino)-1-methylcyclobutyl)carbamate (LXXXIX) (79 mg, 0.170 mmol, 51.1% yield) as a colorless gum. ESIMS found for C₂₃H₂₈N₈O₃ m/z 465.25 (M+H).

### Step 4

Used procedure described in Example 1, Scheme 16, step 4 to produce (1r,3r)-N¹-(5-(imidazo[1,2-a]pyrimidin-6-yl)-4-methoxypyrrolo[2,1-f][1,2,4]triazin-2-yl)-3-methylcyclobutane-1,3-diamine (XC) (45 mg, 0.124 mmol, 72.6% yield) as a white solid. ESIMS found for C₁₈H₂₀N₈O m/z 365.2 (M+H).

### Step 5

To a solution of cyclopropanecarboxylic acid (XCI) (commercially available from Combi-Blocks Inc.) (3 μL, 0.03 mmol) and HATU (14 mg, 0.04 mmol) in DMF (0.5 mL) was added DIPEA (15 μL, 0.09 mmol). The mixture was stirred for 10 min before adding (1r,3r)-N¹-(5-(imidazo[1,2-a]pyrimidin-6-yl)-4-methoxypyrrolo[2,1-f][1,2,4]triazin-2-yl)-3-methylcyclobutane-1,3-diamine (XC) (10 mg, 0.03 mmol) in DMF (0.5 mL). The reaction mixture was stirred at room temperature for 1 h. The reaction mixture was quenched with a saturated aqueous NaHCO₃ solution and extracted with EtOAc. The organics were separated, concentrated, absorbed on silica gel, and purified by ISCO (0→20% MeOH in CHCl₃) to obtain N-((1r,3r)-3-((5-(imidazo[1,2-a]pyrimidin-6-yl)-4-methoxypyrrolo[2,1-f][1,2,4]triazin-2-yl)amino)-1-methylcyclobutyl)cyclopropanecarboxamide (1634) (11 mg, 0.025 mmol, 92.7% yield) as an off-white solid. ¹H NMR (499 MHz, DMSO-d₆) δ ppm 0.57-0.70 (4H, m), 1.36 (3H, s), 1.54-1.64 (1H, m), 1.90-2.02 (2H, m), 2.60-2.70 (2H, m), 3.97 (3H, s), 4.21 (1H, sxt, J=7.83 Hz), 6.77 (1H, d, J=2.46 Hz), 7.06 (1H, d, J=7.39 Hz), 7.67 (1H, d, J=2.46 Hz), 7.72 (1H, d, J=1.37 Hz), 7.93 (1H, d, J=1.37 Hz), 8.15 (1H, s), 8.73 (1H, d, J=2.46 Hz), 9.08 (1H, d, J=2.46 Hz); ESIMS found for C₂₂H₂₄N₈O₂ m/z 433.2 (M+1).

### Example 3

Preparation of (R)-1-(4-((5-(1-(2,2-difluoroethyl)-1H-benzo[d][1,2,3]triazol-6-yl)-4-methoxypyrrolo[2,1-f][1,2,4]triazin-2-yl)amino)-3,3-difluoropiperidin-1-yl)-2-hydroxyethan-1-one (789) are depicted below in Scheme 18.

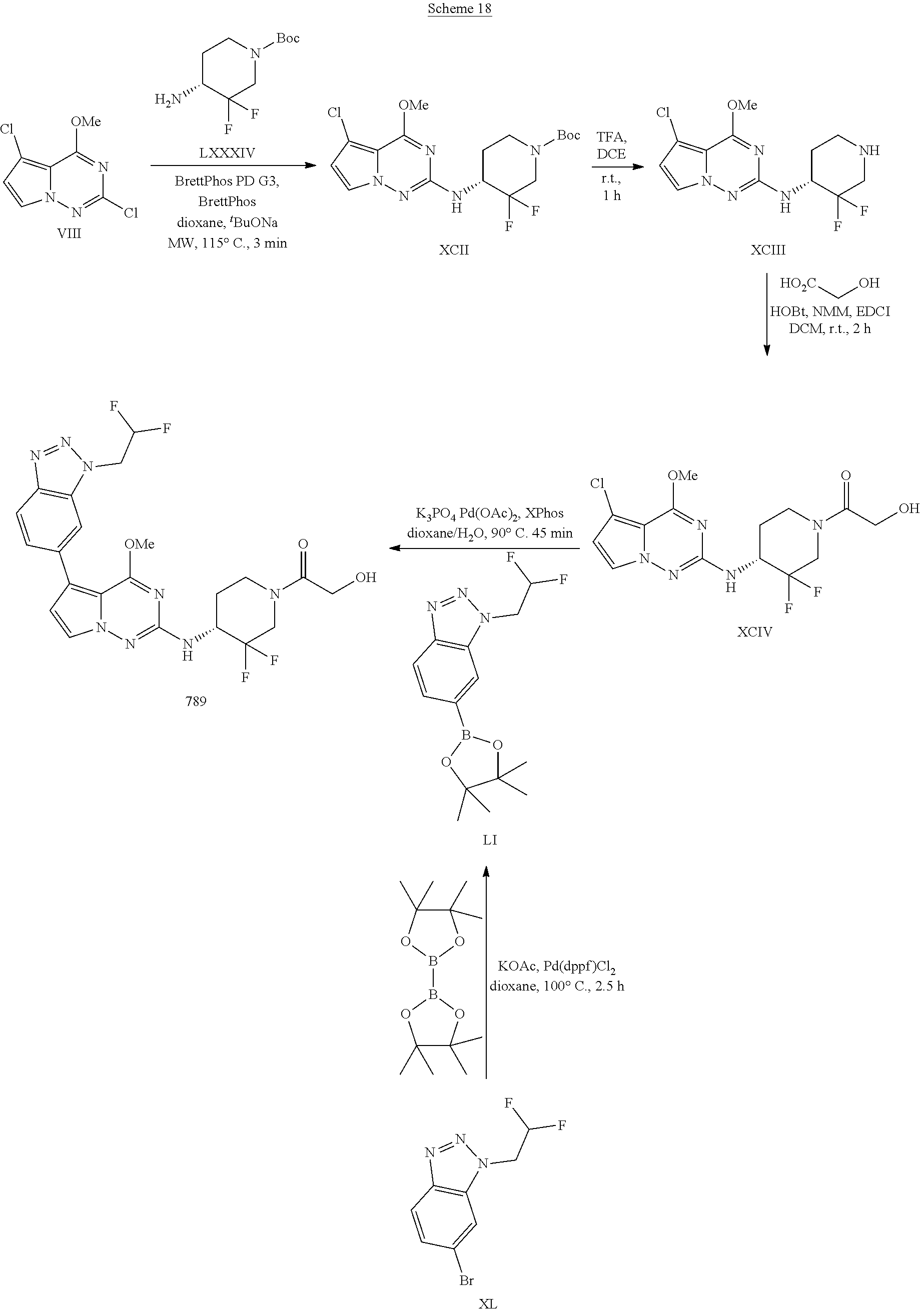

### Steps 1

2,5-dichloro-4-methoxypyrrolo[2,1-f][1,2,4]triazine (VIII) (200 mg, 0.92 mmol), tert-butyl (R)-4-amino-3,3-difluoropiperidine-1-carboxylate (LXXXIV) (260 mg, 1.10 mmol), BrettPhos PD G3 (66 mg, 0.07 mmol), BrettPhos (40 mg, 0.07 mmol) and ^{t}BuONa (220 mg, 2.29 mmol) were suspended in dry 1,4-dioxane (5 mL). 3 Å MS (6 beads) were added and the mixture was stirred at room temperature for 40 min. The beads were removed quickly, and the reaction vessel was sealed and purged and sonicated with Ar for 5 min followed by microwave irradiation at 115° C. for 3 min. The reaction mixture was poured into EtOAc/water, brine was added, and the organic layer was separated. The aqueous layer was extracted with EtOAc (×2) and the combined organic layers were dried (MgSO₄) and reduced in vacuo to give tert-butyl (R)-4-((5-chloro-4-methoxypyrrolo[2,1-f][1,2,4]triazin-2-yl)amino)-3,3-difluoropiperidine-1-carboxylate (XCII) (383 mg, 0.917 mmol, 99.9% yield) as a brown semi-solid which was used without purification. ESIMS found for C₁₇H₂₂ClF₂N₅O₃ m/z 362.1 (M-^{t}Bu+H).

### Step 2

To a solution of tert-butyl (R)-4-((5-chloro-4-methoxypyrrolo[2,1-f][1,2,4]triazin-2-yl)amino)-3,3-difluoropiperidine-1-carboxylate (XCII) (479 mg, 1.15 mmol) in DCE (0.9 mL) was added TFA (0.1 mL) and the reaction was stirred at room temperature for 1 h. The reaction mixture was reduced in vacuo and loaded onto Celite® and purified by column chromatography (0→3% 7.0 M NH₃ in MeOH/CHCl₃) to produce (R)-5-chloro-N-(3,3-difluoropiperidin-4-yl)-4-methoxypyrrolo[2,1-f][1,2,4]triazin-2-amine (XCIII) (177 mg, 0.557 mmol, 48.6% yield) as a pale-yellow solid. ESIMS found for C₁₂H₁₄ClF₂N₅O m/z 318.1 (M+H).

### Step 3

To a suspension of glycolic acid (29 mg, 0.38 mmol) in dry DCM (1 mL) was added (R)-5-chloro-N-(3,3-difluoropiperidin-4-yl)-4-methoxypyrrolo[2,1-f][1,2,4]triazin-2-amine (XCIII) (80 mg, 0.25 mmol), 1-hydroxybenzotriazole (37 mg, 0.27 mmol), EDCI (72 mg, 0.38 mmol) and N-methylmorpholine (61 μL, 0.55 mmol). The reaction was stirred at room temperature for 2 h. The reaction mixture was loaded onto Celite® and purified by column chromatography (0→70% EtOAc/hexanes) to yield (R)-1-(4-((5-chloro-4-methoxypyrrolo[2,1-f][1,2,4]triazin-2-yl)amino)-3,3-difluoropiperidin-1-yl)-2-hydroxyethan-1-one (XCIV) (54 mg, 0.144 mmol, 57.1% yield) as a white semi-solid. ESIMS found for C₁₄H₁₆ClF₂N₅O₃ m/z 376.1 (M+H).

### Step 4

6-Bromo-1-(2,2-difluoroethyl)-1H-benzo[d][1,2,3]triazole (XL) (5.16 g, 19.69 mmol), bis(pinacolato)diboron (7.51 g, 29.57 mmol), Pd(dppf)Cl₂ (803 mg, 0.98 mmol) and KOAc (5.8 g, 59.1 mmol) were suspended in dry 1,4-dioxane (100 mL). The reaction was sonicated and degassed for 5 minutes before heating at 100° C. for 2.5 h. The reaction mixture was loaded onto Celite® and purified by column chromatography (0→50% EtOAc/hexanes) to produce 1-(2,2-difluoroethyl)-6-(4,4,5,5-tetramethyl-1,3,2-dioxaborolan-2-yl)-1H-benzo[d][1,2,3]triazole (LI) (5.654 g, 18.291 mmol, 92.9% yield) as a fluffy pale-orange solid. ESIMS found for C₁₄H₁₈BF₂N₃₀₂ m/z 310.15 (M+H).

### Step 5

To a suspension of (R)-1-(4-((5-chloro-4-methoxypyrrolo[2,1-f][1,2,4]triazin-2-yl)amino)-3,3-difluoropiperidin-1-yl)-2-hydroxyethan-1-one (XCIV) (13 mg, 0.03 mmol), 1-(2,2-difluoroethyl)-6-(4,4,5,5-tetramethyl-1,3,2-dioxaborolan-2-yl)-1H-benzo[d][1,2,3]triazole (LI) (21 mg, 0.07 mmol), Pd(OAc)₂ (2 mg, 0.01 mmol), and XPhos (4 mg, 0.01 mmol) in dry 1,4-dioxane (0.5 mL) was added a 2 M aqueous solution of K₃PO₄ (55 μL, 0.11 mmol). The reaction was sonicated and purged with Ar for 5 min. The reaction was then heated to 90° C. for 45 min. The reaction mixture was loaded onto Celite®, and the product was purified by column chromatography (0→100% EtOAc/hexanes). The product was further purified by HPLC (0→65% MeCN/H₂O in 0.1% formic acid) to give (R)-1-(4-((5-(1-(2,2-Difluoroethyl)-1H-benzo[d][1,2,3]triazol-6-yl)-4-methoxypyrrolo[2,1-f][1,2,4]triazin-2-yl)amino)-3,3-difluoropiperidin-1-yl)-2-hydroxyethan-1-one (789) (8 mg, 0.015 mmol, 44.3% yield) as a fluffy white solid after lyophilization. ¹H NMR (499 MHz, DMSO-d₆) δ ppm 1.60-1.84 (1H, m), 1.87-2.00 (1H, m), 2.99-3.11 (1H, m), 3.59-3.82 (1H, m), 3.98 (3H, s), 4.04-4.31 (3H, m), 4.43-4.65 (2H, m), 4.77-4.87 (1H, m), 5.30 (2H, td, J=15.81, 2.60 Hz), 6.61 (1H, tt, J=54.30, 3.00 Hz), 6.77 (1H, d, J=2.46 Hz), 7.03 (1H, d, J=9.31 Hz), 7.64-7.67 (2H, m), 8.02-8.06 (2H, m); ESIMS found for C₂₂H₂₂F₄N₈O₃ m/z 523.2 (M+1).

### Example 4

Preparation of 2-((3R,4S)-3-fluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-ol (616) are depicted below in Scheme 19.

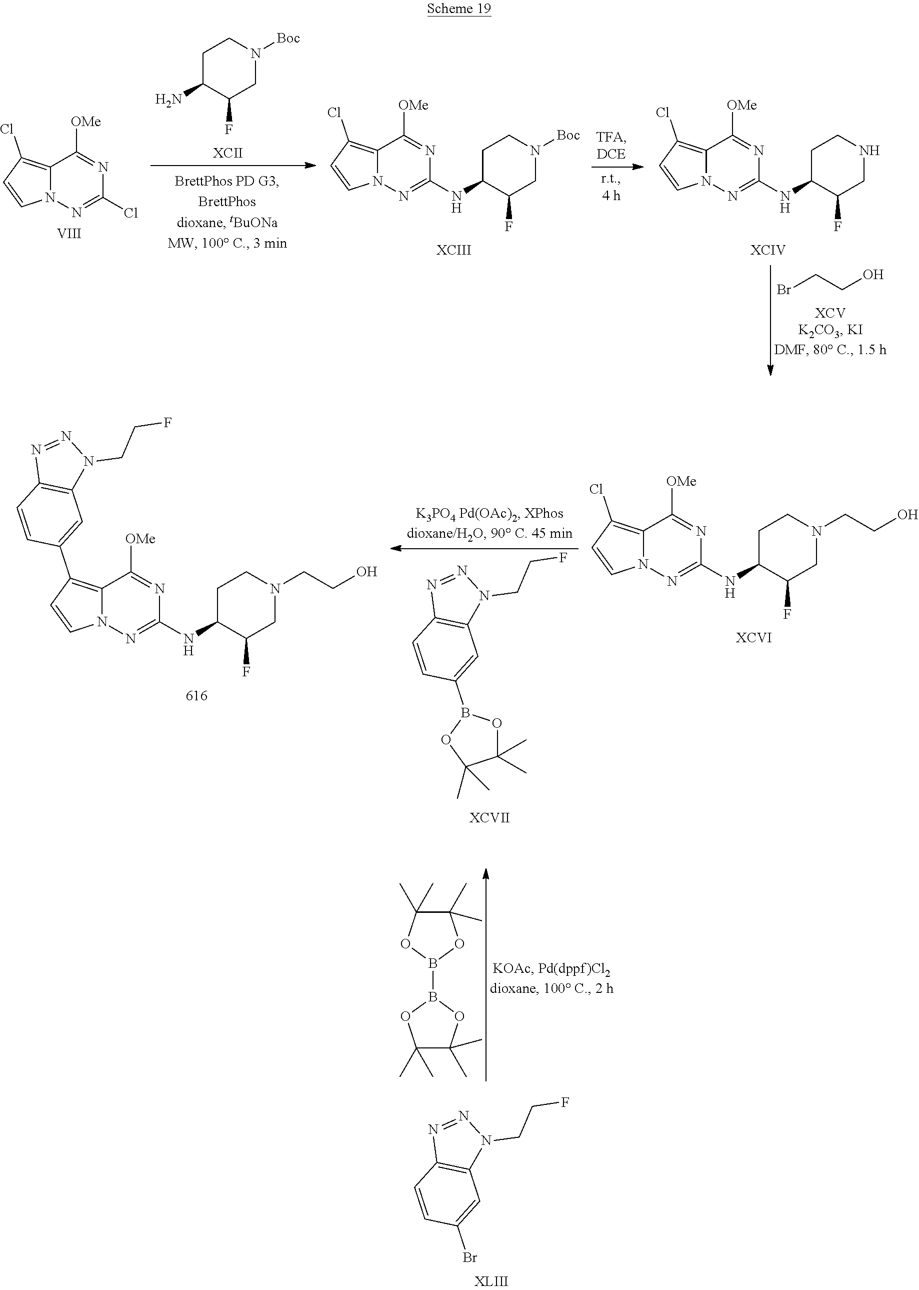

### Steps 1

Used procedure described in Example 3, Scheme 18, step 1 to produce tert-butyl (3R,4S)-4-((5-chloro-4-methoxypyrrolo[2,1-f][1,2,4]triazin-2-yl)amino)-3-fluoropiperidine-1-carboxylate (XCIII). The product was not isolated but used directly in step 2.

### Step 2

Used procedure described in Example 3, Scheme 18, step 2 to produce 5-chloro-N-((3R,4S)-3-fluoropiperidin-4-yl)-4-methoxypyrrolo[2,1-f][1,2,4]triazin-2-amine (XCIV) (342 mg, 1.141 mmol, 71.1% yield for 2 steps) as a light brown solid. ESIMS found for C₁₂H₁₅ClFN₅O m/z 300.1 (M+H).

### Step 3

To a suspension of 5-chloro-N-((3R,4S)-3-fluoropiperidin-4-yl)-4-methoxypyrrolo[2,1-f][1,2,4]triazin-2-amine (XCIV) (342 mg, 1.14 mmol), KI (247 mg, 1.49 mmol) and K₂CO₃ (396 mg, 2.87 mmol) in DMF (6 mL) was added 2-bromoethanol (XCV) (110 μL, 1.55 mmol). The reaction was heated to 80° C. for 1.5 h. The reaction mixture was poured into EtOAc, and water and the organic layer was separated. The aqueous layer was extracted with EtOAc (×2), and the combined organic layers were washed with water, brine, dried (MgSO₄) and reduced in vacuo to give the crude product 2-((3R,4S)-4-((5-chloro-4-methoxypyrrolo[2,1-f][1,2,4]triazin-2-yl)amino)-3-fluoropiperidin-1-yl)ethan-1-ol (XCVI) (294 mg, 0.855 mmol, 74.95% yield) as a beige solid. ESIMS found for C₁₄H₁₉ClFN₅O₂ m/z 344.1 (M+H).

### Step 4

Used procedure described in Example 3, Scheme 18, step 4 to produce 1-(2-fluoroethyl)-6-(4,4,5,5-tetramethyl-1,3,2-dioxaborolan-2-yl)-1H-benzo[d][1,2,3]triazole (XCVII) (5.285 g, 18.153 mmol, 108.2% yield) as a fluffy white solid. ESIMS found for C₁₄H₁₉BFN₃O₂ m/z 292.2 (M+1).

### Step 5

Used procedure described in Example 3, Scheme 18, step 5 to produce 2-((3R,4S)-3-Fluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-ol (616) (27 mg, 0.057 mmol, 39.3% yield) as a white solid. ¹H NMR (499 MHz, DMSO-d₆) δ ppm 1.63-1.74 (1H, m), 1.84-1.99 (1H, m), 2.18 (1H, br t, J=10.81 Hz), 2.32 (1H, dd, J=37.85, 12.59 Hz), 2.44 (2H, t, J=6.30 Hz), 2.91 (1H, br d, J=10.68 Hz), 3.11-3.20 (1H, m), 3.50 (2H, t, J=6.30 Hz), 3.71-3.87 (1H, m), 3.95 (3H, s), 4.41 (1H, br s), 4.90 (1H, d, J=49.90 Hz), 4.94 (2H, dt, J=47.20, 4.65 Hz), 5.08 (2H, dt, J=27.95, 4.65 Hz), 6.62 (1H, d, J=7.94 Hz), 6.75 (1H, d, J=2.74 Hz), 7.60-7.63 (1H, m), 7.64 (1H, d, J=2.46 Hz), 7.99-8.03 (2H, m); ESIMS found for C₂₂H₂₆F₂N₈O₂ m/z 473.2 (M+1).

The following compounds were prepared in accordance with the procedures described in the above Schemes 1-19.

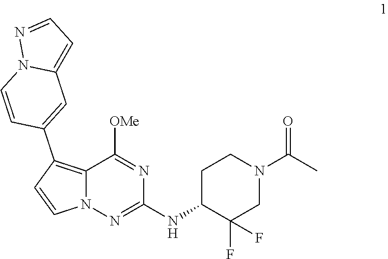 [SMILES: COc1nc(N[C@@H]2CCN(C(C)=O)CC2(F)F)nn2ccc(-c3ccn4nccc4c3)c12]

(R)-1-(3,3-Difluoro-4-((4-methoxy-5-(pyrazolo[1,5-a]pyridin-5-yl)pyrrolo [2,1-f][1,2,4]triazin-2-yl)amino)piperidin-1-yl)ethan-1-one 1.

Off-white solid (24 mg, 0.054 mmol, 86.9% yield). ¹H NMR (499 MHz, DMSO-d₆) δ ppm 1.57-1.83 (1H, m), 1.84-2.01 (1H, m), 2.02-2.09 (3H, m), 2.98 (1H, br t, J=11.36 Hz), 3.61-3.93 (1H, m), 4.02 (3H, s), 4.08-4.32 (1H, m), 4.44-4.62 (2H, m), 6.59 (1H, d, J=1.64 Hz), 6.81 (1H, d, J=2.46 Hz), 7.01 (1H, d, J=9.31 Hz), 7.11 (1H, dd, J=7.12, 1.92 Hz), 7.63 (1H, dd, J=2.46, 1.37 Hz), 7.79-7.86 (1H, m), 7.97 (1H, d, J=2.19 Hz), 8.63 (1H, d, J=7.39 Hz); ESIMS found for C₂₁H₂₁F₂N₇O₂ m/z 442.1 (M+1).

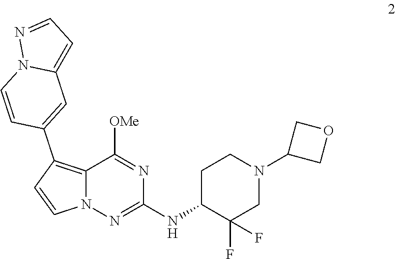 [SMILES: COc1nc(N[C@@H]2CCN(C3COC3)CC2(F)F)nn2ccc(-c3ccn4nccc4c3)c12]

(R)—N-(3,3-Difluoro-1-(oxetan-3-yl)piperidin-4-yl)-4-methoxy-5-(pyrazolo [1,5-a]pyridin-5-yl)pyrrolo[2,1-f][1,2,4]triazin-2-amine 2.

Off-white solid (28 mg, 0.062 mmol, 81.8% yield). ¹H NMR (499 MHz, DMSO-d₆) δ ppm 1.75-1.86 (1H, m), 1.86-1.92 (1H, m), 2.16 (1H, br t, J=10.54 Hz), 2.40 (1H, dd, J=26.85, 11.77 Hz), 2.76 (1H, br d, J=12.05 Hz), 2.95-3.09 (1H, m), 3.60 (1H, quin, J=6.37 Hz), 4.02 (3H, s), 4.22-4.37 (1H, m), 4.44 (2H, dt, J=15.13, 6.26 Hz), 4.55 (2H, td, J=6.57, 3.56 Hz), 6.59 (1H, d, J=1.64 Hz), 6.80 (1H, d, J=2.74 Hz), 6.91 (1H, d, J=9.31 Hz), 7.11 (1H, dd, J=7.26, 2.05 Hz), 7.63 (1H, d, J=2.46 Hz), 7.82 (1H, d, J=1.09 Hz), 7.97 (1H, d, J=2.46 Hz), 8.63 (1H, d, J=7.12 Hz); ESIMS found for C₂₂H₂₃F₂N₇O₂ m/z 456.2 (M+1).

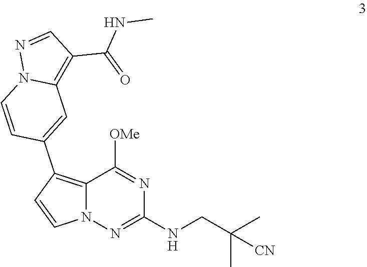 [SMILES: CNC(=O)c1cnn2ccc(-c3ccn4nc(NCC(C)(C)C#N)nc(OC)c34)cc12]

5-(2-((2-Cyano-2-methylpropyl)amino)-4-methoxypyrrolo[2,1-f][1,2,4]triazin-5-yl)-N-methylpyrazolo[1,5-a]pyridine-3-carboxamide 3.

Beige solid (21 mg, 0.050 mmol, 46.8% yield). ¹H NMR (499 MHz, DMSO-d₆) δ ppm 1.37 (6H, s), 2.79 (3H, d, J=4.38 Hz), 3.49 (2H, d, J=6.84 Hz), 4.07 (3H, s), 6.88 (1H, d, J=2.46 Hz), 7.19 (1H, t, J=6.57 Hz), 7.30 (1H, dd, J=7.26, 2.05 Hz), 7.67 (1H, d, J=2.74 Hz), 8.11 (1H, q, J=4.38 Hz), 8.46 (1H, s), 8.53 (1H, d, J=1.37 Hz), 8.67-8.72 (1H, m); ESIMS found for C₂₁H₂₂N₈O₂ m/z 419.2 (M+1).

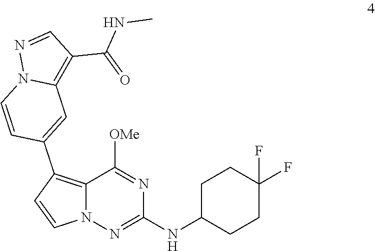 [SMILES: CNC(=O)c1cnn2ccc(-c3ccn4nc(NC5CCC(F)(F)CC5)nc(OC)c34)cc12]

5-(2-((4,4-Difluorocyclohexyl)amino)-4-methoxypyrrolo[2,1-f][1,2,4]triazin-5-yl)-N-methylpyrazolo[1,5-a]pyridine-3-carboxamide 4.

Beige solid (28 mg, 0.062 mmol, 64.9% yield). ¹H NMR (499 MHz, DMSO-d₆) δ ppm 1.55-1.70 (2H, m), 1.85-2.03 (4H, m), 2.08 (2H, q, J=10.59 Hz), 2.79 (3H, d, J=4.65 Hz), 3.81 (1H, br d, J=8.49 Hz), 4.03 (3H, s), 6.82 (1H, d, J=7.94 Hz), 6.85 (1H, d, J=2.74 Hz), 7.29 (1H, dd, J=7.39, 2.19 Hz), 7.65 (1H, d, J=2.74 Hz), 8.10 (1H, q, J=4.56 Hz), 8.46 (1H, s), 8.52 (1H, dd, J=1.92, 0.82 Hz), 8.69 (1H, dd, J=7.39, 0.82 Hz); ESIMS found for C₂₂H₂₃F₂N₇O₂ m/z 456.2 (M+1).

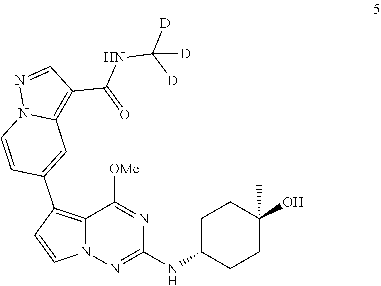 [SMILES: [2H]C([2H])([2H])NC(=O)c1cnn2ccc(-c3ccn4nc(N[C@H]5CC[C@](C)(O)CC5)nc(OC)c34)cc12]

5-(2-(((1r,4r)-4-Hydroxy-4-methylcyclohexyl)amino)-4-methoxypyrrolo[2,1-f][1,2,4]triazin-5-yl)-N-(methyl-d₃)pyrazolo[1,5-a]pyridine-3-carboxamide 5.

Off-white solid (35 mg, 0.077 mmol, 60.1% yield). ¹H NMR (499 MHz, DMSO-d₆) δ ppm 1.15 (3H, s), 1.36-1.52 (4H, m), 1.55-1.66 (2H, m), 1.81-1.95 (2H, m), 3.65 (1H, br dd, J=7.94, 3.83 Hz), 4.02 (3H, s), 4.24 (1H, s), 6.56 (1H, d, J=7.94 Hz), 6.83 (1H, d, J=2.74 Hz), 7.29 (1H, dd, J=7.26, 2.05 Hz), 7.66 (1H, d, J=2.46 Hz), 8.07 (1H, s), 8.45 (1H, s), 8.52 (1H, d, J=1.37 Hz), 8.68 (1H, d, J=7.12 Hz); ESIMS found for C₂₃H₂₄[2H₃]N₇O₃ m/z 453.25 (M+1).

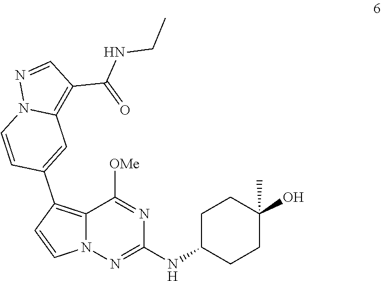 [SMILES: CCNC(=O)c1cnn2ccc(-c3ccn4nc(N[C@H]5CC[C@](C)(O)CC5)nc(OC)c34)cc12]

N-Ethyl-5-(2-(((1r,4r)-4-hydroxy-4-methylcyclohexyl)amino)-4-methoxypyrrolo[2,1-f][1,2,4]triazin-5-yl)pyrazolo[1,5-a]pyridine-3-carboxamide 6.

Beige solid (33 mg, 0.071 mmol, 55.3% yield). ¹H NMR (499 MHz, DMSO-d₆) δ ppm 1.14 (3H, t, J=7.25 Hz), 1.14 (3H, s), 1.36-1.53 (4H, m), 1.55-1.65 (2H, m), 1.81-1.93 (2H, m), 3.27-3.32 (2H, m), 3.64 (1H, br dd, J=7.94, 4.11 Hz), 4.02 (3H, s), 4.24 (1H, s), 6.56 (1H, d, J=7.94 Hz), 6.83 (1H, d, J=2.74 Hz), 7.29 (1H, dd, J=7.26, 2.05 Hz), 7.65 (1H, d, J=2.74 Hz), 8.12 (1H, t, J=5.61 Hz), 8.49 (1H, s), 8.52 (1H, d, J=1.37 Hz), 8.68 (1H, d, J=7.12 Hz); ESIMS found for C₂₄H₂₉N₇O₃ m/z 464.25 (M+1).

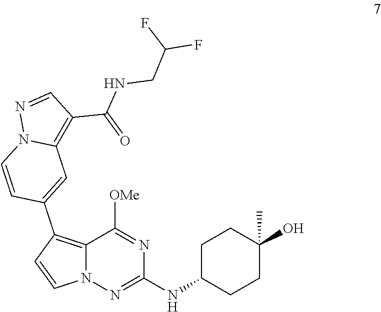 [SMILES: COc1nc(N[C@H]2CC[C@](C)(O)CC2)nn2ccc(-c3ccn4ncc(C(=O)NCC(F)F)c4c3)c12]

N-(2,2-Difluoroethyl)-5-(2-(((1r,4r)-4-hydroxy-4-methylcyclohexyl)amino)-4-methoxypyrrolo[2,1-f][1,2,4]triazin-5-yl)pyrazolo[1,5-a]pyridine-3-carboxamide 7.

Beige solid (24 mg, 0.048 mmol, 37.3% yield). ¹H NMR (499 MHz, DMSO-d₆) δ ppm 1.14 (3H, s), 1.35-1.55 (4H, m), 1.56-1.65 (2H, m), 1.82-1.92 (2H, m), 3.60-3.77 (3H, m), 4.02 (3H, s), 4.24 (1H, br s), 6.13 (1H, tt, J=56.25, 3.97 Hz), 6.57 (1H, d, J=7.94 Hz), 6.85 (1H, d, J=2.74 Hz), 7.33 (1H, dd, J=7.26, 2.05 Hz), 7.66 (1H, d, J=2.74 Hz), 8.51-8.56 (2H, m), 8.57 (1H, s), 8.72 (1H, dd, J=7.39, 0.82 Hz); ESIMS found for C₂₄H₂₇F₂N₇O₃ m/z 250.6 (M/2+1).

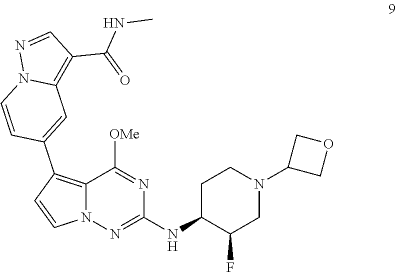 [SMILES: CNC(=O)c1cnn2ccc(-c3ccn4nc(N[C@H]5CCN(C6COC6)C[C@H]5F)nc(OC)c34)cc12]

5-(2-(((3R,4S)-3-Fluoro-1-(oxetan-3-yl)piperidin-4-yl)amino)-4-methoxypyrrolo[2,1-f][1,2,4]triazin-5-yl)-N-methylpyrazolo[1,5-a]pyridine-3-carboxamide 9.

Off-white solid (7 mg, 0.014 mmol, 33.6% yield). ¹H NMR (499 MHz, DMSO-d₆) δ ppm 1.72 (1H, br d, J=12.59 Hz), 1.92 (1H, qd, J=11.91, 3.15 Hz), 1.98-2.06 (1H, m), 2.16 (1H, dd, J=37.05, 12.32 Hz), 2.76 (1H, br d, J=12.05 Hz), 2.79 (3H, d, J=4.38 Hz), 2.98 (1H, br d, J=9.31 Hz), 3.46-3.53 (1H, m), 3.75-3.89 (1H, m), 4.04 (3H, s), 4.40 (1H, t, J=6.02 Hz), 4.46 (1H, t, J=6.02 Hz), 4.54 (2H, td, J=6.50, 2.87 Hz), 4.94 (1H, d, J=48.80 Hz), 6.77 (1H, d, J=7.94 Hz), 6.86 (1H, d, J=2.46 Hz), 7.29 (1H, dd, J=7.39, 2.19 Hz), 7.65 (1H, d, J=2.74 Hz), 8.11 (1H, q, J=4.56 Hz), 8.46 (1H, s), 8.50-8.55 (1H, m), 8.67-8.73 (1H, m); ESIMS found for C₂₄H₂₇FN₈O₃ m/z 495.3 (M+1).

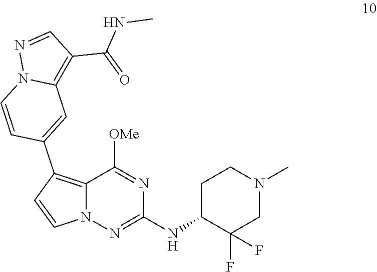 [SMILES: CNC(=O)c1cnn2ccc(-c3ccn4nc(N[C@@H]5CCN(C)CC5(F)F)nc(OC)c34)cc12]

(R)-5-(2-((3,3-Difluoro-1-methylpiperidin-4-yl)amino)-4-methoxypyrrolo [2,1-f][1,2,4]triazin-5-yl)-N-methylpyrazolo[1,5-a]pyridine-3-carboxamide 10.

Off-white solid (9 mg, 0.019 mmol, 25.4% yield). ¹H NMR (499 MHz, DMSO-d₆) δ ppm 1.78-1.92 (2H, m), 2.12-2.23 (1H, m), 2.26 (3H, s), 2.33-2.44 (1H, m), 2.76-2.81 (1H, m), 2.79 (2H, d, J=4.65 Hz), 3.01-3.12 (1H, m), 4.05 (3H, s), 4.16-4.33 (1H, m), 6.87 (1H, d, J=2.74 Hz), 6.90 (1H, d, J=9.31 Hz), 7.30 (1H, dd, J=7.26, 2.05 Hz), 7.67 (1H, d, J=2.46 Hz), 8.10 (1H, q, J=4.47 Hz), 8.46 (1H, s), 8.50-8.56 (1H, m), 8.69 (1H, dd, J=7.12, 0.82 Hz) ESIMS found for C₂₂H₂₄F₂N₈O₂ m/z 471.25 (M+1).

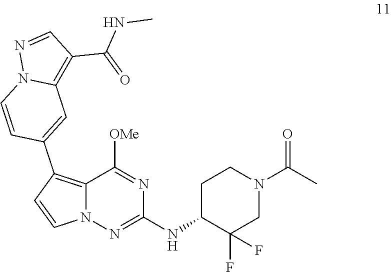 [SMILES: CNC(=O)c1cnn2ccc(-c3ccn4nc(N[C@@H]5CCN(C(C)=O)CC5(F)F)nc(OC)c34)cc12]

(R)-5-(2-((1-Acetyl-3,3-difluoropiperidin-4-yl)amino)-4-methoxypyrrolo [2,1-f][1,2,4]triazin-5-yl)-N-methylpyrazolo[1,5-a]pyridine-3-carboxamide 11.

Beige solid (22 mg, 0.044 mmol, 56.7% yield). ¹H NMR (499 MHz, DMSO-d₆) δ ppm 1.57-1.83 (1H, m), 1.89-1.99 (1H, m), 2.02-2.10 (3H, m), 2.79 (3H, d, J=4.38 Hz), 3.70 (1H, dd, J=28.55, 14.24 Hz), 3.87 (1H, br d, J=13.96 Hz), 4.06 (3H, s), 4.13-4.32 (1H, m), 4.44-4.63 (2H, m), 6.89 (1H, d, J=2.74 Hz), 7.07 (1H, d, J=9.31 Hz), 7.30 (1H, dd, J=7.26, 2.05 Hz), 7.66 (1H, dd, J=2.60, 1.51 Hz), 8.07-8.16 (1H, m), 8.46 (1H, s), 8.50-8.57 (1H, m), 8.70 (1H, dd, J=7.12, 0.82 Hz); ESIMS found for C₂₃H₂₄F₂N₈O₃ m/z 499.1 (M+1).

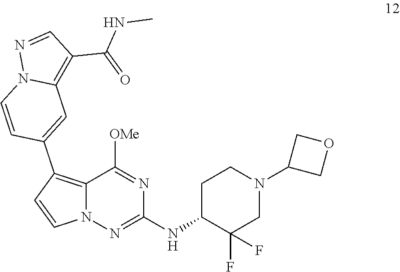 [SMILES: CNC(=O)c1cnn2ccc(-c3ccn4nc(N[C@@H]5CCN(C6COC6)CC5(F)F)nc(OC)c34)cc12]

(R)-5-(2-((3,3-Difluoro-1-(oxetan-3-yl)piperidin-4-yl)amino)-4-methoxypyrrolo[2,1-f][1,2,4]triazin-5-yl)-N-methylpyrazolo[1,5-a]pyridine-3-carboxamide 12.

Beige solid (11 mg, 0.022 mmol, 25.1% yield). ¹H NMR (499 MHz, DMSO-d₆) δ ppm 1.76-1.86 (1H, m), 1.87-1.94 (1H, m), 2.16 (1H, br t, J=10.68 Hz), 2.41 (1H, dd, J=27.40, 12.05 Hz), 2.76 (1H, br d, J=11.77 Hz), 2.79 (3H, d, J=4.38 Hz), 2.96-3.08 (1H, m), 3.56-3.65 (1H, m), 4.06 (3H, s), 4.24-4.38 (1H, m), 4.44 (2H, dt, J=15.13, 6.26 Hz), 4.55 (2H, td, J=6.57, 3.56 Hz), 6.87 (1H, d, J=2.46 Hz), 6.97 (1H, d, J=9.58 Hz), 7.30 (1H, dd, J=7.39, 2.19 Hz), 7.66 (1H, d, J=2.46 Hz), 8.11 (1H, q, J=4.29 Hz), 8.46 (1H, s), 8.50-8.54 (1H, m), 8.66-8.73 (1H, m); ESIMS found for C₂₄H₂₆F₂N₈O₃ m/z 513.2 (M+1).

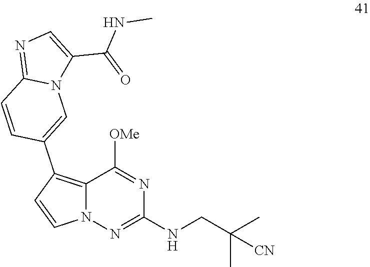 [SMILES: CNC(=O)c1cnc2ccc(-c3ccn4nc(NCC(C)(C)C#N)nc(OC)c34)cn12]

6-(2-((2-Cyano-2-methylpropyl)amino)-4-methoxypyrrolo[2,1-f][1,2,4]triazin-5-yl)-N-methylimidazo[1,2-a]pyridine-3-carboxamide 41.

Beige solid (32 mg, 0.077 mmol, 71.3% yield). ¹H NMR (499 MHz, DMSO-d₆) δ ppm 1.36 (6H, s), 2.83 (3H, d, J=4.65 Hz), 3.49 (2H, d, J=6.57 Hz), 4.05 (3H, s), 6.80 (1H, d, J=2.74 Hz), 7.14 (1H, t, J=6.71 Hz), 7.66 (1H, d, J=2.74 Hz), 7.69 (2H, d, J=1.37 Hz), 8.27 (1H, s), 8.44 (1H, q, J=4.29 Hz), 9.82 (1H, t, J=1.37 Hz); ESIMS found for C₂₁H₂₂N₈O₂ m/z 419.2 (M+1).

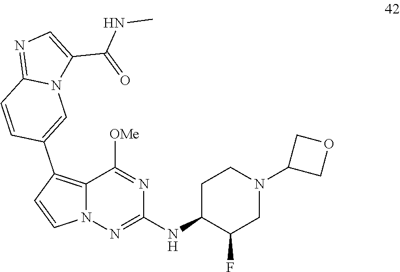 [SMILES: CNC(=O)c1cnc2ccc(-c3ccn4nc(N[C@H]5CCN(C6COC6)C[C@H]5F)nc(OC)c34)cn12]

6-(2-(((3R,4S)-3-Fluoro-1-(oxetan-3-yl)piperidin-4-yl)amino)-4-methoxypyrrolo[2,1-f][1,2,4]triazin-5-yl)-N-methylimidazo[1,2-a]pyridine-3-carboxamide 42.

Off-white solid (12 mg, 0.024 mmol, 54.0% yield). ¹H NMR (499 MHz, DMSO-d₆) δ ppm 1.72 (1H, br dd, J=9.17, 3.97 Hz), 1.92 (1H, qd, J=12.09, 3.70 Hz), 1.98-2.06 (1H, m), 2.16 (1H, dd, J=36.45, 12.35 Hz), 2.76 (1H, br d, J=9.86 Hz), 2.82 (3H, d, J=4.38 Hz), 2.93-3.02 (1H, m), 3.49 (1H, quin, J=6.30 Hz), 3.73-3.92 (1H, m), 4.02 (3H, s), 4.40 (1H, t, J=6.16 Hz), 4.46 (1H, t, J=6.02 Hz), 4.54 (2H, td, J=6.57, 3.01 Hz), 4.94 (1H, d, J=49.90 Hz), 6.72 (1H, d, J=7.94 Hz), 6.78 (1H, d, J=2.74 Hz), 7.64 (1H, d, J=2.74 Hz), 7.69 (2H, d, J=1.37 Hz), 8.27 (1H, s), 8.44 (1H, q, J=4.38 Hz), 9.81 (1H, t, J=1.37 Hz); ESIMS found for C₂₄H₂₇FN₈O₃ m/z 495.2 (M+1).

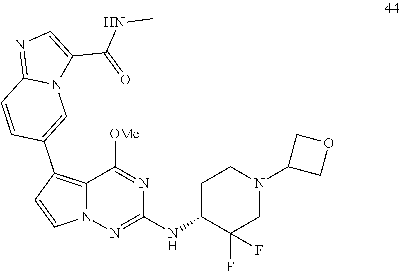 [SMILES: CNC(=O)c1cnc2ccc(-c3ccn4nc(N[C@@H]5CCN(C6COC6)CC5(F)F)nc(OC)c34)cn12]

(R)-6-(2-((3,3-Difluoro-1-(oxetan-3-yl)piperidin-4-yl)amino)-4-methoxypyrrolo[2,1-f][1,2,4]triazin-5-yl)-N-methylimidazo[1,2-a]pyridine-3-carboxamide 44.

White solid (11 mg, 0.022 mmol, 25.1% yield). ¹H NMR (499 MHz, DMSO-d₆) δ ppm 1.75-1.86 (1H, m), 1.86-1.94 (1H, m), 2.11-2.20 (1H, m), 2.40 (1H, dd, J=26.60, 11.77 Hz), 2.76 (1H, br d, J=11.77 Hz), 2.83 (3H, d, J=4.65 Hz), 2.96-3.07 (1H, m), 3.60 (1H, quin, J=6.23 Hz), 4.04 (3H, s), 4.24-4.37 (1H, m), 4.44 (2H, dt, J=15.19, 6.09 Hz), 4.55 (2H, td, J=6.57, 3.56 Hz), 6.79 (1H, d, J=2.74 Hz), 6.92 (1H, d, J=9.58 Hz), 7.65 (1H, d, J=2.46 Hz), 7.69 (2H, d, J=1.37 Hz), 8.27 (1H, s), 8.44 (1H, q, J=4.47 Hz), 9.81 (1H, t, J=1.37 Hz); ESIMS found for C₂₄H₂₆F₂N₈O₃ m/z 513.3 (M+1).

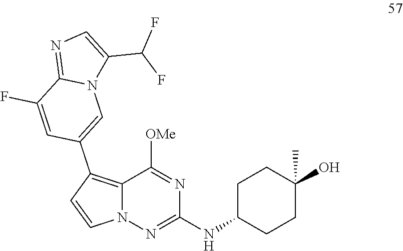 [SMILES: COc1nc(N[C@H]2CC[C@](C)(O)CC2)nn2ccc(-c3cc(F)c4ncc(C(F)F)n4c3)c12]

(1r,4r)-4-((5-(3-(Difluoromethyl)-8-fluoroimidazo[1,2-a]pyridin-6-yl)-4-methoxypyrrolo[2,1-f][1,2,4]triazin-2-yl)amino)-1-methylcyclohexan-1-ol 57.

Off-white solid (21 mg, 0.046 mmol, 28.3% yield). ¹H NMR (499 MHz, DMSO-d₆) δ ppm 1.14 (3H, s), 1.36-1.52 (4H, m), 1.56-1.68 (2H, m), 1.82-1.94 (2H, m), 3.64 (1H, br dd, J=8.08, 3.97 Hz), 3.99 (3H, s), 4.23 (1H, s), 6.56 (1H, d, J=7.94 Hz), 6.85 (1H, d, J=2.74 Hz), 7.60 (1H, t, J=52.95 Hz), 7.67 (1H, d, J=2.74 Hz), 7.69 (1H, dd, J=12.10, 0.85 Hz), 7.99 (1H, s), 8.65 (1H, d, J=1.37 Hz); ESIMS found for C₂₂H₂₃F₃N₆O₂ m/z 461.2 (M+1).

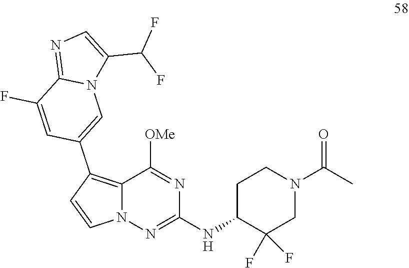 [SMILES: COc1nc(N[C@@H]2CCN(C(C)=O)CC2(F)F)nn2ccc(-c3cc(F)c4ncc(C(F)F)n4c3)c12]

(R)-1-(4-((5-(3-(Difluoromethyl)-8-fluoroimidazo[1,2-a]pyridin-6-yl)-4-methoxypyrrolo[2,1-f][1,2,4]triazin-2-yl)amino)-3,3-difluoropiperidin-1-yl)ethan-1-one 58.

Off-white solid (18 mg, 0.035 mmol, 42.4% yield). ¹H NMR (499 MHz, DMSO-d₆) δ ppm 1.57-1.84 (1H, m), 1.84-1.99 (1H, m), 2.07 (3H, s), 2.92-3.05 (1H, m), 3.65-3.92 (1H, m), 4.03 (3H, s), 4.11-4.31 (1H, m), 4.44-4.61 (2H, m), 6.91 (1H, d, J=2.74 Hz), 7.08 (1H, br d, J=9.03 Hz), 7.60 (1H, t, J=52.95 Hz), 7.68 (1H, dd, J=2.60, 1.51 Hz), 7.71 (1H, d, J=12.32 Hz), 7.99 (1H, s), 8.66 (1H, d, J=1.10 Hz); ESIMS found for C₂₂H₂₀F₅N₇O₂ m/z 510.2 (M+1).

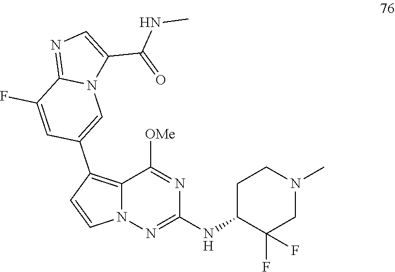 [SMILES: CNC(=O)c1cnc2c(F)cc(-c3ccn4nc(N[C@@H]5CCN(C)CC5(F)F)nc(OC)c34)cn12]

(R)-6-(2-((3,3-Difluoro-1-methylpiperidin-4-yl)amino)-4-methoxypyrrolo[2,1-f][1,2,4]triazin-5-yl)-8-fluoro-N-methylimidazo[1,2-a]pyridine-3-carboxamide 76.

Off-white solid (7 mg, 0.014 mmol, 36.6% yield). ¹H NMR (499 MHz, DMSO-d₆) δ ppm 1.76-1.92 (2H, m), 2.13-2.22 (1H, m), 2.26 (3H, s), 2.34-2.43 (1H, m), 2.77 (1H, br s), 2.84 (3H, d, J=4.38 Hz), 3.01-3.09 (1H, m), 4.06 (3H, s), 4.17-4.32 (1H, m), 6.85 (1H, d, J=2.74 Hz), 6.89 (1H, d, J=9.31 Hz), 7.66 (1H, d, J=2.74 Hz), 7.69 (1H, dd, J=12.32, 1.37 Hz), 8.30 (1H, s), 8.54-8.59 (1H, m), 9.70 (1H, d, J=1.37 Hz); ESIMS found for C₂₂H₂₃F₃N₈O₂ m/z 489.15 (M+1).

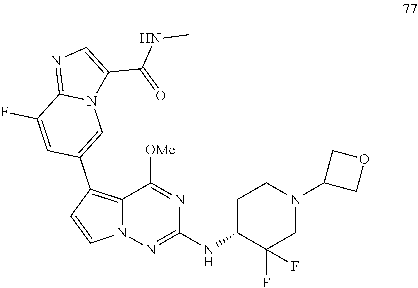 [SMILES: CNC(=O)c1cnc2c(F)cc(-c3ccn4nc(N[C@@H]5CCN(C6COC6)CC5(F)F)nc(OC)c34)cn12]

(R)-6-(2-((3,3-Difluoro-1-(oxetan-3-yl)piperidin-4-yl)amino)-4-methoxypyrrolo[2,1-f][1,2,4]triazin-5-yl)-8-fluoro-N-methylimidazo[1,2-a]pyridine-3-carboxamide 77.

Beige solid (5 mg, 0.028 mmol, 48.0% yield). ¹H NMR (499 MHz, DMSO-d₆) δ ppm 1.76-1.86 (1H, m), 1.86-1.93 (1H, m), 2.16 (1H, br t, J=10.27 Hz), 2.40 (1H, dd, J=27.45, 12.32 Hz), 2.76 (1H, br d, J=12.32 Hz), 2.84 (3H, d, J=4.65 Hz), 2.95-3.08 (1H, m), 3.60 (1H, quin, J=6.30 Hz), 4.07 (3H, s), 4.24-4.36 (1H, m), 4.44 (2H, dt, J=15.19, 6.09 Hz), 4.55 (2H, td, J=6.57, 3.56 Hz), 6.85 (1H, d, J=2.46 Hz), 6.96 (1H, d, J=9.31 Hz), 7.66 (1H, d, J=2.74 Hz), 7.69 (1H, dd, J=12.46, 1.23 Hz), 8.30 (1H, s), 8.56 (1H, q, J=4.38 Hz), 9.70 (1H, d, J=1.37 Hz); ESIMS found for C₂₄H₂₅F₃N₈O₃ m/z 531.2 (M+1).

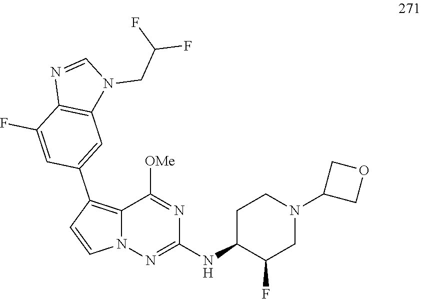 [SMILES: COc1nc(N[C@H]2CCN(C3COC3)C[C@H]2F)nn2ccc(-c3cc(F)c4ncn(CC(F)F)c4c3)c12]

5-(1-(2,2-Difluoroethyl)-4-fluoro-1H-benzo[d]imidazol-6-yl)-N-((3R,4S)-3-fluoro-1-(oxetan-3-yl)piperidin-4-yl)-4-methoxypyrrolo[2,1-f][1,2,4]triazin-2-amine 271.

White solid (12 mg, 0.023 mmol, 23.5% yield). ¹H NMR (499 MHz, DMSO-d₆) δ ppm 1.66-1.78 (1H, m), 1.92 (1H, qd, J=12.05, 3.29 Hz), 1.98-2.07 (1H, m), 2.15 (1H, dd, J=36.75, 12.87 Hz), 2.76 (1H, br d, J=10.68 Hz), 2.94-3.03 (1H, m), 3.49 (1H, quin, J=6.43 Hz), 3.74-3.90 (1H, m), 3.96 (3H, d, J=1.10 Hz), 4.40 (1H, t, J=6.02 Hz), 4.46 (1H, t, J=6.16 Hz), 4.54 (2H, td, J=6.50, 3.15 Hz), 4.79-4.88 (2H, m), 4.93 (1H, d, J=49.90 Hz), 6.50 (1H, tt, J=54.30, 2.87 Hz), 6.64 (1H, t, J=8.08 Hz), 6.70 (1H, t, J=2.46 Hz), 7.24-7.36 (1H, m), 7.60 (1H, dd, J=8.35, 2.60 Hz), 7.66-7.70 (1H, m), 8.26 (1H, d, J=2.19 Hz); ESIMS found for C₂₄H₂₅F₄N₇O₂ m/z 260.6 (M/2+1).

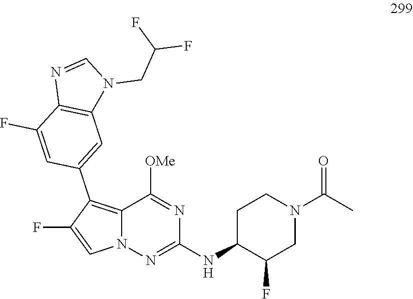 [SMILES: COc1nc(N[C@H]2CCN(C(C)=O)C[C@H]2F)nn2cc(F)c(-c3cc(F)c4ncn(CC(F)F)c4c3)c12]

1-((3R,4S)-4-((5-(1-(2,2-Difluoroethyl)-4-fluoro-1H-benzo[d]imidazol-6-yl)-6-fluoro-4-methoxypyrrolo[2,1-f][1,2,4]triazin-2-yl)amino)-3-fluoropiperidin-1-yl)ethan-1-one 299.

White solid (15 mg, 0.029 mmol, 55.2% yield). ¹H NMR (499 MHz, DMSO-d₆) δ ppm 1.64-1.73 (1H, m), 1.74-1.90 (1H, m), 1.97-2.07 (3H, m), 2.66-2.98 (1H, m), 3.15-3.25 (1H, m), 3.92 (3H, s), 3.94-4.05 (1H, m), 4.06-4.16 (1H, m), 4.38-4.75 (1H, m), 4.86 (2H, tdd, J=15.98, 15.98, 7.73, 2.74 Hz), 4.97 (1H, d, J=49.90 Hz), 6.52 (1H, tt, J=54.30, 2.87 Hz), 6.84-6.89 (1H, m), 7.26 (1H, d, J=12.59 Hz), 7.61 (1H, s), 7.78 (1H, br d, J=2.74 Hz), 8.30 (1H, s); ESIMS found for C₂₃H₂₂F₅N₇O₂ m/z 524.2 (M+1).

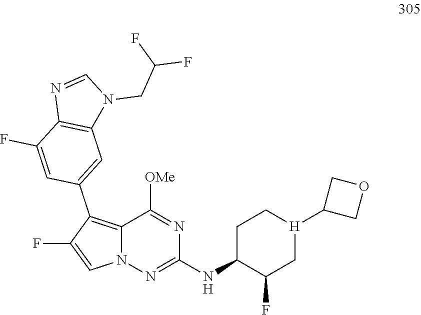

5-(1-(2,2-Difluoroethyl)-4-fluoro-1H-benzo[d]imidazol-6-yl)-6-fluoro-N-((3R,4S)-3-fluoro-1-(oxetan-3-yl)piperidin-4-yl)-4-methoxypyrrolo[2,1-f][1,2,4]triazin-2-amine 305.

White solid (16 mg, 0.030 mmol, 57.3% yield). ¹H NMR (499 MHz, DMSO-d₆) δ ppm 1.64-1.78 (1H, m), 1.91 (1H, qd, J=12.14, 3.29 Hz), 1.97-2.05 (1H, m), 2.14 (1H, dd, J=37.00, 12.59 Hz), 2.75 (1H, br d, J=10.13 Hz), 2.98 (1H, br t, J=10.54 Hz), 3.49 (1H, quin, J=6.37 Hz), 3.71-3.87 (1H, m), 3.92 (3H, s), 4.40 (1H, t, J=6.16 Hz), 4.46 (1H, t, J=6.16 Hz), 4.54 (2H, td, J=6.43, 3.01 Hz), 4.80-4.91 (2H, m), 4.91 (1H, d, J=49.90 Hz), 6.51 (1H, tt, J=54.30, 2.87 Hz), 6.76 (1H, t, J=7.53 Hz), 7.26 (1H, d, J=12.59 Hz), 7.62 (1H, d, J=12.05 Hz), 7.78 (1H, br d, J=3.01 Hz), 8.29 (1H, s); ESIMS found for C₂₄H₂₄F₅N₇O₂ m/z 538.2 (M+1).

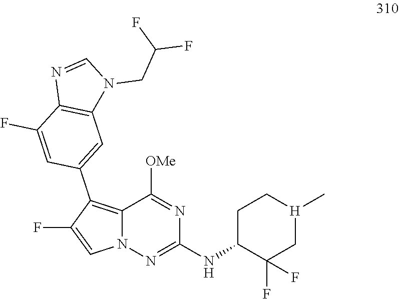

(R)—N-(3,3-Difluoro-1-methylpiperidin-4-yl)-5-(1-(2,2-difluoroethyl)-4-fluoro-1H-benzo[d]imidazol-6-yl)-6-fluoro-4-methoxypyrrolo[2,1-f][1,2,4]triazin-2-amine 310.

White solid (8 mg, 0.016 mmol, 31.1% yield). ¹H NMR (499 MHz, DMSO-d₆) δ ppm 1.75-1.91 (2H, m), 2.16 (1H, td, J=11.09, 3.29 Hz), 2.25 (3H, s), 2.37 (1H, dd, J=27.15, 11.77 Hz), 2.78 (1H, br d, J=11.23 Hz), 2.98-3.10 (1H, m), 3.93 (3H, s), 4.13-4.29 (1H, m), 4.85 (2H, tdd, J=15.95, 15.95, 8.08, 2.74 Hz), 6.51 (1H, tt, J=54.55, 2.74 Hz), 6.86-6.95 (1H, m), 7.15-7.31 (1H, m), 7.57-7.67 (1H, m), 7.81 (1H, dd, J=5.20, 2.74 Hz), 8.30 (1H, d, J=4.93 Hz); ESIMS found for C₂₂H₂₁F₆N₇O m/z 257.6 (M/2+1).

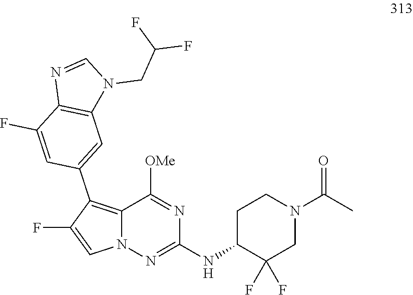 [SMILES: COc1nc(N[C@@H]2CCN(C(C)=O)CC2(F)F)nn2cc(F)c(-c3cc(F)c4ncn(CC(F)F)c4c3)c12]

(R)-1-(4-((5-(1-(2,2-Difluoroethyl)-4-fluoro-1H-benzo[d]imidazol-6-yl)-6-fluoro-4-methoxypyrrolo[2,1-f][1,2,4]triazin-2-yl)amino)-3,3-difluoropiperidin-1-yl)ethan-1-one 313.

White solid (9 mg, 0.017 mmol, 33.2% yield). ¹H NMR (499 MHz, DMSO-d₆) δ ppm 1.56-1.82 (1H, m), 1.84-1.98 (1H, m), 2.07 (3H, s), 2.92-3.04 (1H, m), 3.61-3.90 (1H, m), 3.94 (3H, s), 4.10-4.30 (1H, m), 4.42-4.58 (2H, m), 4.76-4.95 (2H, m), 6.52 (1H, tt, J=54.55, 2.87 Hz), 7.02-7.12 (1H, m), 7.27 (1H, d, J=12.59 Hz), 7.62 (1H, s), 7.79 (1H, d, J=2.46 Hz), 8.30 (1H, s); ESIMS found for C₂₃H₂₁F₆N₇O₂ m/z 542.15 (M+1).

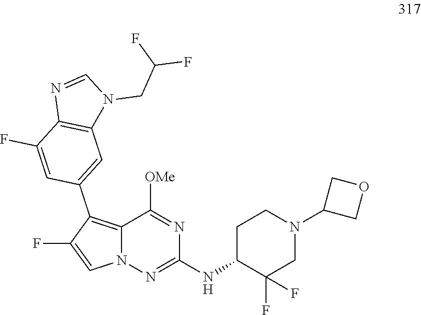 [SMILES: COc1nc(N[C@@H]2CCN(C3COC3)CC2(F)F)nn2cc(F)c(-c3cc(F)c4ncn(CC(F)F)c4c3)c12]

(R)—N-(3,3-Difluoro-1-(oxetan-3-yl)piperidin-4-yl)-5-(1-(2,2-difluoroethyl)-4-fluoro-1H-benzo[d]imidazol-6-yl)-6-fluoro-4-methoxypyrrolo[2,1-f][1,2,4]triazin-2-amine 317.

White solid (7 mg, 0.013 mmol, 25.2% yield). ¹H NMR (499 MHz, DMSO-d₆) δ ppm 1.76-1.85 (1H, m), 1.85-1.92 (1H, m), 2.09-2.20 (1H, m), 2.39 (1H, dd, J=26.90, 11.77 Hz), 2.76 (1H, br d, J=11.50 Hz), 2.96-3.07 (1H, m), 3.60 (1H, quin, J=6.30 Hz), 3.94 (3H, s), 4.22-4.36 (1H, m), 4.44 (2H, dt, J=15.06, 6.16 Hz), 4.55 (2H, td, J=6.57, 3.83 Hz), 4.77-4.93 (2H, m), 6.51 (1H, tt, J=54.55, 2.75 Hz), 6.95 (1H, d, J=9.03 Hz), 7.26 (1H, d, J=12.59 Hz), 7.61 (1H, s), 7.80 (1H, br d, J=3.01 Hz), 8.29 (1H, s); ESIMS found for C₂₄H₂₃F₆N₇O₂ m/z 556.2 (M+1).

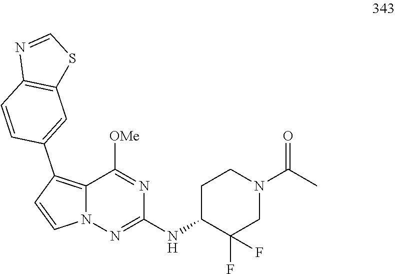 [SMILES: COc1nc(N[C@@H]2CCN(C(C)=O)CC2(F)F)nn2ccc(-c3ccc4ncsc4c3)c12]

(R)-1-(4-((5-(Benzo[d]thiazol-6-yl)-4-methoxypyrrolo[2,1-f][1,2,4]triazin-2-yl)amino)-3,3-difluoropiperidin-1-yl)ethan-1-one 343.

White solid (22 mg, 0.048 mmol, 34.5% yield). ¹H NMR (499 MHz, DMSO-d₆) δ ppm 1.56-1.84 (1H, m), 1.86-2.01 (1H, m), 2.03-2.10 (3H, m), 2.92-3.07 (1H, m), 3.63-3.93 (1H, m), 3.98 (3H, s), 4.09-4.32 (1H, m), 4.44-4.63 (2H, m), 6.75 (1H, d, J=2.74 Hz), 6.97 (1H, d, J=9.31 Hz), 7.63 (1H, dd, J=2.60, 1.23 Hz), 7.74 (1H, dd, J=8.49, 1.64 Hz), 8.07 (1H, d, J=8.49 Hz), 8.30 (1H, d, J=1.64 Hz), 9.36 (1H, s); ESIMS found for C₂₁H₂₀F₂N₆O₂S m/z 459.1 (M+1).

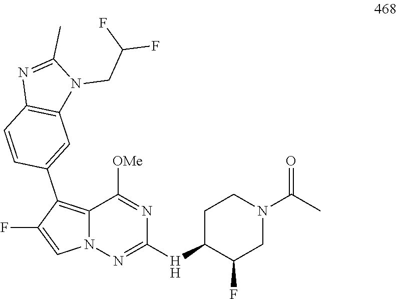

1-((3R,4S)-4-((5-(1-(2,2-Difluoroethyl)-2-methyl-1H-benzo[d]imidazol-6-yl)-6-fluoro-4-methoxypyrrolo[2,1-f][1,2,4]triazin-2-yl)amino)-3-fluoropiperidin-1-yl)ethan-1-one 468.

White solid (2.6 mg, 0.005 mmol, 77.1% yield). ¹H NMR (499 MHz, DMSO-d₆) δ ppm 1.64-1.72 (1H, m), 1.73-1.91 (1H, m), 1.98-2.07 (3H, m), 2.58 (3H, s), 2.67-2.96 (1H, m), 3.14-3.25 (1H, m), 3.89 (3H, s), 3.92-4.07 (1H, m), 4.08-4.19 (1H, m), 4.39-4.52 (1H, m), 4.75 (2H, td, J=16.15, 2.19 Hz), 4.97 (1H, d, J=49.95 Hz), 6.46 (1H, tt, J=54.30, 2.87 Hz), 6.76-6.85 (1H, m), 7.30 (1H, d, J=8.49 Hz), 7.55 (1H, d, J=8.49 Hz), 7.65 (1H, s), 7.76 (1H, d, J=3.01 Hz); ESIMS found for C₂₄H₂₅F₄N₇O₂ m/z 520.2 (M+1).

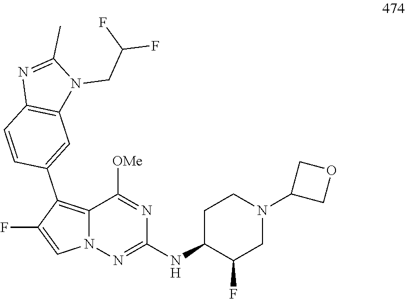 [SMILES: COc1nc(N[C@H]2CCN(C3COC3)C[C@H]2F)nn2cc(F)c(-c3ccc4nc(C)n(CC(F)F)c4c3)c12]

5-(1-(2,2-Difluoroethyl)-2-methyl-1H-benzo[d]imidazol-6-yl)-6-fluoro-N-((3R,4S)-3-fluoro-1-(oxetan-3-yl)piperidin-4-yl)-4-methoxypyrrolo[2,1-f][1,2,4]triazin-2-amine 474.

White solid (14.4 mg, 0.027 mmol, 51.5% yield). ¹H NMR (499 MHz, DMSO-d₆) δ ppm 1.72 (1H, br d, J=10.13 Hz), 1.91 (1H, qd, J=12.14, 3.29 Hz), 1.98-2.06 (1H, m), 2.15 (1H, dd, J=36.20, 11.77 Hz), 2.57 (3H, s), 2.76 (1H, br d, J=10.68 Hz), 2.93-3.06 (1H, m), 3.43-3.55 (1H, m), 3.70-3.85 (1H, m), 3.89 (3H, s), 4.40 (1H, t, J=6.02 Hz), 4.46 (1H, t, J=6.02 Hz), 4.54 (2H, td, J=6.43, 2.74 Hz), 4.74 (2H, td, J=16.15, 2.19 Hz), 4.92 (1H, d, J=49.65 Hz), 6.45 (1H, tt, J=54.30, 2.87 Hz), 6.70 (1H, br d, J=7.67 Hz), 7.28 (1H, d, J=8.21 Hz), 7.54 (1H, d, J=8.21 Hz), 7.64 (1H, s), 7.76 (1H, d, J=2.74 Hz); ESIMS found for C₂₅H₂₇F₄N₇O₂ m/z 534.1 (M+1).

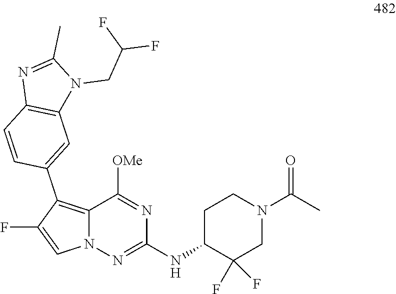

(R)-1-(4-((5-(1-(2,2-Difluoroethyl)-2-methyl-1H-benzo[d]imidazol-6-yl)-6-fluoro-4-methoxypyrrolo[2,1-f][1,2,4]triazin-2-yl)amino)-3,3-difluoropiperidin-1-yl)ethan-1-one 482.

White solid (0.4 mg, 0.0007 mmol, 18.4% yield). ¹H NMR (499 MHz, DMSO-d₆) δ ppm 1.56-1.82 (1H, m), 1.83-1.99 (1H, m), 2.03-2.09 (3H, m), 2.60 (3H, s), 2.98 (1H, br t, J=10.95 Hz), 3.62-3.88 (1H, m), 3.91 (3H, s), 4.11-4.31 (1H, m), 4.42-4.60 (2H, m), 4.77 (2H, td, J=15.90, 2.20 Hz), 6.47 (1H, tt, J=54.30, 2.87 Hz), 7.02 (1H, d, J=9.86 Hz), 7.33 (1H, s), 7.57 (1H, d, J=8.21 Hz), 7.68 (1H, s), 7.78 (1H, s); ESIMS found for C₂₄H₂₄F₅N₇O₂ m/z 538.2 (M+1).

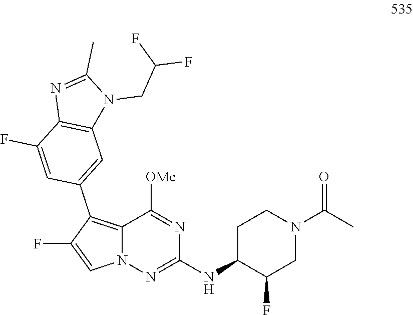 [SMILES: COc1nc(N[C@H]2CCN(C(C)=O)C[C@H]2F)nn2cc(F)c(-c3cc(F)c4nc(C)n(CC(F)F)c4c3)c12]

1-((3R,4S)-4-((5-(1-(2,2-Difluoroethyl)-4-fluoro-2-methyl-1H-benzo[d]imidazol-6-yl)-6-fluoro-4-methoxypyrrolo[2,1-f][1,2,4]triazin-2-yl)amino)-3-fluoropiperidin-1-yl)ethan-1-one 535.

White solid (9 mg, 0.017 mmol, 51.9% yield). ¹H NMR (499 MHz, DMSO-d₆) δ ppm 1.64-1.72 (1H, m), 1.73-1.90 (1H, m), 2.00 (3H, s), 2.59 (3H, s), 2.65-2.97 (1H, m), 3.21 (1H, br t, J=11.91 Hz), 3.85-4.01 (1H, m), 3.91 (2H, s), 4.02-4.17 (1H, m), 4.38-4.72 (1H, m), 4.77 (2H, td, J=16.08, 2.33 Hz), 4.97 (1H, d, J=49.10 Hz), 6.47 (1H, tt, J=54.60, 2.87 Hz), 6.85 (1H, dd, J=7.67, 3.56 Hz), 7.12 (1H, d, J=12.05 Hz), 7.52 (1H, s), 7.78 (1H, d, J=2.74 Hz); ESIMS found for C₂₄H₂₄F₅N₇O₂ m/z 538.2 (M+1).

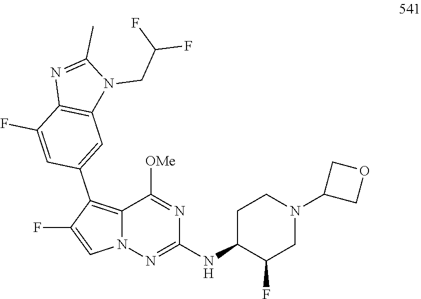

5-(1-(2,2-Difluoroethyl)-4-fluoro-2-methyl-1H-benzo[d]imidazol-6-yl)-6-fluoro-N-((3R,4S)-3-fluoro-1-(oxetan-3-yl)piperidin-4-yl)-4-methoxypyrrolo[2,1-f][1,2,4]triazin-2-amine 541.

White solid (8 mg, 0.015 mmol, 44.9% yield). ¹H NMR (499 MHz, DMSO-d₆) δ ppm 1.63-1.77 (1H, m), 1.91 (1H, qd, J=12.14, 3.29 Hz), 1.97-2.05 (1H, m), 2.14 (1H, dd, J=37.00, 12.59 Hz), 2.75 (1H, br d, J=10.68 Hz), 2.92-3.04 (1H, m), 3.49 (1H, quin, J=6.37 Hz), 3.69-3.87 (1H, m), 3.91 (3H, s), 4.40 (1H, t, J=6.02 Hz), 4.46 (1H, t, J=6.16 Hz), 4.54 (2H, td, J=6.50, 2.87 Hz), 4.77 (2H, td, J=16.20, 2.46 Hz), 4.91 (1H, d, J=49.90 Hz), 6.47 (1H, tt, J=54.30, 2.87 Hz), 6.75 (1H, d, J=7.67 Hz), 7.11 (1H, d, J=11.77 Hz), 7.51 (1H, s), 7.78 (1H, d, J=3.01 Hz); ESIMS found for C₂₅H₂₆F₅N₇O₂ m/z 552.2 (M+1).

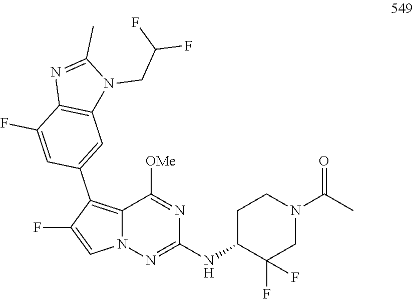 [SMILES: COc1nc(N[C@@H]2CCN(C(C)=O)CC2(F)F)nn2cc(F)c(-c3cc(F)c4nc(C)n(CC(F)F)c4c3)c12]

(R)-1-(4-((5-(1-(2,2-Difluoroethyl)-4-fluoro-2-methyl-1H-benzo[d]imidazol-6-yl)-6-fluoro-4-methoxypyrrolo[2,1-f][1,2,4]triazin-2-yl)amino)-3,3-difluoropiperidin-1-yl)ethan-1-one 549.

White solid (5 mg, 0.009 mmol, 42.0% yield). ¹H NMR (499 MHz, DMSO-d₆) δ ppm 1.57-1.83 (1H, m), 1.83-1.99 (1H, m), 2.07 (3H, s), 2.59 (3H, s), 2.91-3.05 (1H, m), 3.61-3.90 (1H, m), 3.93 (3H, s), 4.11-4.31 (1H, m), 4.40-4.60 (2H, m), 4.78 (2H, td, J=16.15, 2.19 Hz), 6.47 (1H, tt, J=54.50, 2.87 Hz), 7.06 (1H, dd, J=9.31, 1.92 Hz), 7.12 (1H, d, J=12.05 Hz), 7.52 (1H, s), 7.74-7.81 (1H, m); ESIMS found for C₂₄H₂₃F₆N₇O₂ m/z 556.2 (M+1).

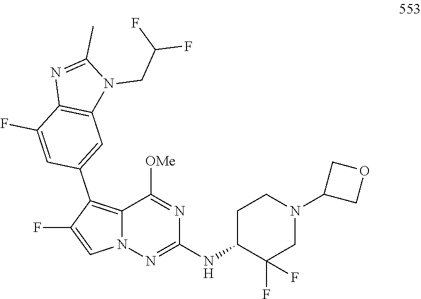 [SMILES: COc1nc(N[C@@H]2CCN(C3COC3)CC2(F)F)nn2cc(F)c(-c3cc(F)c4nc(C)n(CC(F)F)c4c3)c12]

(R)—N-(3,3-Difluoro-1-(oxetan-3-yl)piperidin-4-yl)-5-(1-(2,2-difluoroethyl)-4-fluoro-2-methyl-1H-benzo[d]imidazol-6-yl)-6-fluoro-4-methoxypyrrolo[2,1-f][1,2,4]triazin-2-amine 553.

White solid (4 mg, 0.007 mmol, 32.8% yield). ¹H NMR (499 MHz, DMSO-d₆) δ ppm 1.76-1.85 (1H, m), 1.85-1.92 (1H, m), 2.15 (1H, br t, J=10.68 Hz), 2.39 (1H, dd, J=27.15, 11.50 Hz), 2.59 (3H, s), 2.76 (1H, br d, J=10.40 Hz), 2.94-3.08 (1H, m), 3.60 (1H, quin, J=6.23 Hz), 3.89-3.98 (3H, m), 4.20-4.35 (1H, m), 4.44 (2H, dt, J=15.19, 6.09 Hz), 4.55 (2H, td, J=6.57, 3.83 Hz), 4.77 (2H, td, J=16.45, 2.19 Hz), 6.47 (1H, tt, J=54.30, 2.74 Hz), 6.96 (1H, d, J=9.31 Hz), 7.12 (1H, d, J=11.77 Hz), 7.52 (1H, s), 7.79 (1H, d, J=3.01 Hz); ESIMS found for C₂₅H₂₅F₆N₇O₂ m/z 570.2 (M+1).

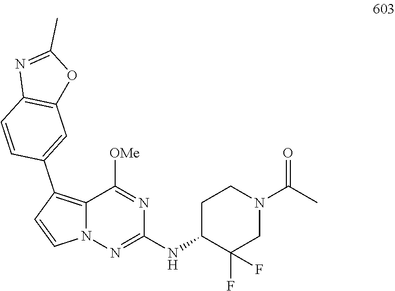 [SMILES: COc1nc(N[C@@H]2CCN(C(C)=O)CC2(F)F)nn2ccc(-c3ccc4nc(C)oc4c3)c12]

(R)-1-(3,3-Difluoro-4-((4-methoxy-5-(2-methylbenzo[d]oxazol-6-yl)pyrrolo[2,1-f][1,2,4]triazin-2-yl)amino)piperidin-1-yl)ethan-1-one 603.

White solid (12 mg, 0.026 mmol, 27.0% yield). ¹H NMR (499 MHz, DMSO-d₆) δ ppm 1.56-1.83 (1H, m), 1.84-2.01 (1H, m), 2.02-2.09 (3H, m), 2.62 (3H, s), 2.93-3.05 (1H, m), 3.63-3.91 (1H, m), 3.98 (3H, s), 4.09-4.31 (1H, m), 4.41-4.61 (2H, m), 6.71 (1H, d, J=2.46 Hz), 6.95 (1H, d, J=9.58 Hz), 7.51 (1H, dd, J=8.21, 1.37 Hz), 7.59-7.64 (2H, m), 7.80 (1H, d, J=1.37 Hz); ESIMS found for C₂₂H₂₂F₂N₆O₃ m/z 457.2 (M+1).

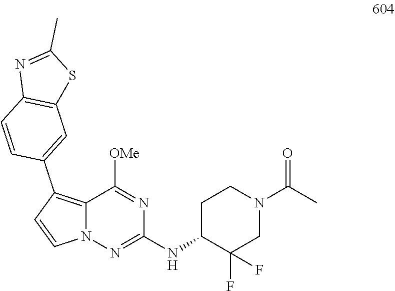 [SMILES: COc1nc(N[C@@H]2CCN(C(C)=O)CC2(F)F)nn2ccc(-c3ccc4nc(C)sc4c3)c12]

(R)-1-(3,3-Difluoro-4-((4-methoxy-5-(2-methylbenzo[d]thiazol-6-yl)pyrrolo[2,1-f][1,2,4]triazin-2-yl)amino)piperidin-1-yl)ethan-1-one 604.

White solid (14 mg, 0.030 mmol, 30.5% yield). ¹H NMR (499 MHz, DMSO-d₆) δ ppm 1.56-1.84 (1H, m), 1.84-2.00 (1H, m), 2.03-2.10 (3H, m), 2.81 (3H, s), 2.98 (1H, br t, J=11.09 Hz), 3.63-3.92 (1H, m), 3.97 (3H, s), 4.09-4.33 (1H, m), 4.44-4.61 (2H, m), 6.72 (1H, d, J=2.46 Hz), 6.95 (1H, d, J=9.58 Hz), 7.62 (1H, dd, J=2.46, 1.37 Hz), 7.66 (1H, dd, J=8.49, 1.64 Hz), 7.88 (1H, d, J=8.21 Hz), 8.16 (1H, d, J=1.92 Hz); ESIMS found for C₂₂H₂₂F₂N₆O₂S m/z 473.2 (M+1).

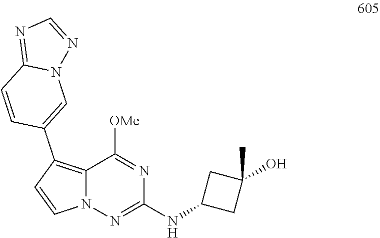 [SMILES: COc1nc(N[C@H]2C[C@@](C)(O)C2)nn2ccc(-c3ccc4ncnn4c3)c12]

(1s,3s)-3-((5-([1,2,4]Triazolo[1,5-a]pyridin-6-yl)-4-methoxypyrrolo[2,1-f][1,2,4]triazin-2-yl)amino)-1-methylcyclobutan-1-ol 605.

Beige solid (17 mg, 0.047 mmol, 28.0% yield). ¹H NMR (499 MHz, DMSO-d₆) δ ppm 1.27 (3H, s), 1.97-2.07 (2H, m), 2.31-2.40 (2H, m), 3.67-3.77 (1H, m), 3.96 (3H, s), 4.93 (1H, s), 6.80 (1H, d, J=2.46 Hz), 6.97 (1H, d, J=6.57 Hz), 7.65 (1H, d, J=2.74 Hz), 7.82-7.86 (1H, m), 7.87-7.91 (1H, m), 8.49 (1H, s), 9.06 (1H, d, J=0.82 Hz); ESIMS found for C₁₈H₁₉N₇O₂ m/z 366.2 (M+1).

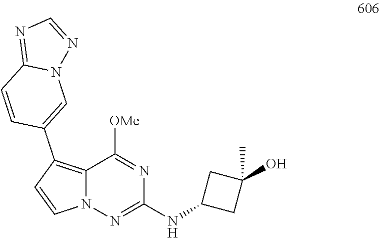 [SMILES: COc1nc(N[C@H]2C[C@](C)(O)C2)nn2ccc(-c3ccc4ncnn4c3)c12]

(1r,3r)-3-((5-([1,2,4]Triazolo[1,5-a]pyridin-6-yl)-4-methoxypyrrolo[2,1-f][1,2,4]triazin-2-yl)amino)-1-methylcyclobutan-1-ol 606.

Off-white solid (13 mg, 0.036 mmol, 21.4% yield). ¹H NMR (499 MHz, DMSO-d₆) δ ppm 1.28 (3H, s), 1.94-2.05 (2H, m), 2.27-2.36 (2H, m), 3.96 (3H, s), 4.25 (1H, dq, J=14.78, 7.30 Hz), 4.79 (1H, s), 6.80 (1H, d, J=2.46 Hz), 6.98 (1H, d, J=6.84 Hz), 7.65 (1H, d, J=2.74 Hz), 7.82-7.85 (1H, m), 7.87-7.91 (1H, m), 8.49 (1H, s), 9.02-9.09 (1H, m); ESIMS found for C₁₈H₁₉N₇O₂ m/z 366.15 (M+1).

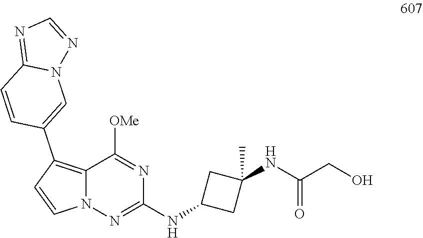 [SMILES: COc1nc(N[C@H]2C[C@](C)(NC(=O)CO)C2)nn2ccc(-c3ccc4ncnn4c3)c12]

N-((1r,3r)-3-((5-([1,2,4]Triazolo[1,5-a]pyridin-6-yl)-4-methoxypyrrolo[2,1-f][1,2,4]triazin-2-yl)amino)-1-methylcyclobutyl)-2-hydroxyacetamide 607.

Off-white solid (4 mg, 0.010 mmol, 31.4% yield). ¹H NMR (499 MHz, DMSO-d₆) δ ppm 1.39 (3H, s), 1.93-2.06 (2H, m), 2.68-2.74 (2H, m), 3.79 (2H, br d, J=4.11 Hz), 3.96 (3H, s), 4.19 (1H, dq, J=15.30, 7.77 Hz), 5.35-5.44 (1H, m), 6.80 (1H, d, J=2.46 Hz), 7.05 (1H, d, J=7.12 Hz), 7.60 (1H, s), 7.65 (1H, d, J=2.74 Hz), 7.81-7.85 (1H, m), 7.87-7.91 (1H, m), 8.49 (1H, s), 9.04-9.09 (1H, m); ESIMS found for C₂₀H₂₂N₈O₃ m/z 423.2 (M+1).

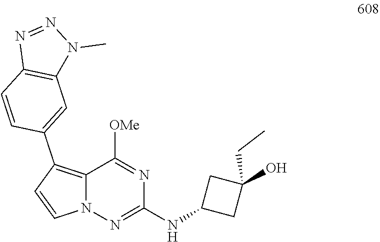 [SMILES: CC[C@]1(O)C[C@H](Nc2nc(OC)c3c(-c4ccc5nnn(C)c5c4)ccn3n2)C1]

(1r,3s)-1-Ethyl-3-((4-methoxy-5-(1-methyl-1H-benzo[d][1,2,3]triazol-6-yl)pyrrolo[2,1-f][1,2,4]triazin-2-yl)amino)cyclobutan-1-ol 608.

Off-white solid (29 mg, 0.074 mmol, 40.0% yield). ¹H NMR (499 MHz, DMSO-d₆) δ ppm 0.82 (3H, t, J=7.39 Hz), 1.51 (2H, q, J=7.39 Hz), 1.90-2.00 (2H, m), 2.20-2.30 (2H, m), 3.95 (3H, s), 4.25-4.34 (1H, m), 4.31 (3H, s), 4.62 (1H, s), 6.74 (1H, d, J=2.46 Hz), 6.93 (1H, d, J=7.12 Hz), 7.60 (1H, dd, J=8.62, 1.51 Hz), 7.63 (1H, d, J=2.74 Hz), 7.93 (1H, s), 7.97 (1H, d, J=8.49 Hz); ESIMS found for C₂₀H₂₃N₇O₂ m/z 394.2 (M+1).

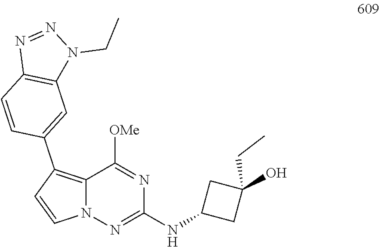 [SMILES: CCn1nnc2ccc(-c3ccn4nc(N[C@H]5C[C@@](O)(CC)C5)nc(OC)c34)cc21]

(1r,3s)-1-Ethyl-3-((5-(1-ethyl-1H-benzo[d][1,2,3]triazol-6-yl)-4-methoxypyrrolo[2,1-f][1,2,4]triazin-2-yl)amino)cyclobutan-1-ol 609.

Off-white solid (20 mg, 0.049 mmol, 32.3% yield). ¹H NMR (499 MHz, DMSO-d₆) δ ppm 0.82 (3H, t, J=7.39 Hz), 1.51 (2H, q, J=7.39 Hz), 1.54 (3H, t, J=7.26 Hz), 1.90-2.00 (2H, m), 2.19-2.30 (2H, m), 3.94 (3H, s), 4.30 (1H, sxt, J=7.39 Hz), 4.62 (1H, s), 4.75 (2H, q, J=7.39 Hz), 6.75 (1H, d, J=2.74 Hz), 6.93 (1H, d, J=6.84 Hz), 7.58-7.61 (1H, m), 7.63 (1H, d, J=2.74 Hz), 7.98 (1H, br d, J=8.21 Hz), 7.97-7.98 (1H, m); ESIMS found for C₂₁H₂₅N₇O₂ m/z 408.2 (M+1).

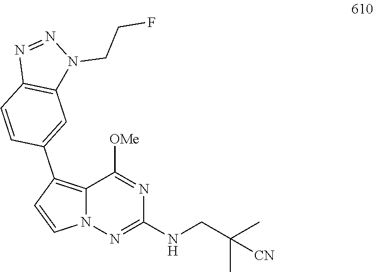 [SMILES: COc1nc(NCC(C)(C)C#N)nn2ccc(-c3ccc4nnn(CCF)c4c3)c12]

3-((5-(1-(2-Fluoroethyl)-1H-benzo[d][1,2,3]triazol-6-yl)-4-methoxypyrrolo[2,1-f][1,2,4]triazin-2-yl)amino)-2,2-dimethylpropanenitrile 610.

White solid (31 mg, 0.076 mmol, 70.8% yield). ¹H NMR (499 MHz, DMSO-d₆) δ ppm 1.36 (6H, s), 3.49 (2H, d, J=6.57 Hz), 3.98 (3H, s), 4.94 (2H, dt, J=47.20, 4.65 Hz), 5.08 (2H, dt, J=28.00, 4.65 Hz), 6.77 (1H, d, J=2.46 Hz), 7.12 (1H, t, J=6.57 Hz), 7.61-7.64 (1H, m), 7.66 (1H, d, J=2.46 Hz), 8.01 (1H, s), 8.02 (1H, d, J=6.84 Hz); ESIMS found for C₂₀H₂₁FN₈O m/z 409.2 (M+1).

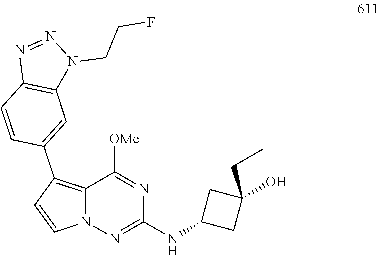 [SMILES: CC[C@]1(O)C[C@@H](Nc2nc(OC)c3c(-c4ccc5nnn(CCF)c5c4)ccn3n2)C1]

(1s,3r)-1-Ethyl-3-((5-(1-(2-fluoroethyl)-1H-benzo[d][1,2,3]triazol-6-yl)-4-methoxypyrrolo[2,1-f][1,2,4]triazin-2-yl)amino)cyclobutan-1-ol 611.

Beige solid (11 mg, 0.026 mmol, 19.2% yield). ¹H NMR (499 MHz, DMSO-d₆) δ ppm 0.87 (3H, t, J=7.26 Hz), 1.50 (2H, q, J=7.39 Hz), 1.91-2.01 (2H, m), 2.41 (2H, ddd, J=9.51, 7.32, 2.60 Hz), 3.62-3.72 (1H, m), 3.93 (3H, s), 4.75 (1H, s), 4.94 (2H, dt, J=47.20, 4.65 Hz), 5.08 (2H, dt, J=28.00, 4.65 Hz), 6.73 (1H, d, J=2.74 Hz), 6.93 (1H, d, J=6.57 Hz), 7.61 (1H, dd, J=8.62, 1.51 Hz), 7.64 (1H, d, J=2.46 Hz), 7.99 (1H, s), 8.00 (1H, d, J=8.76 Hz); ESIMS found for C₂₁H₂₄FN₇O₂ m/z 426.2 (M+1).

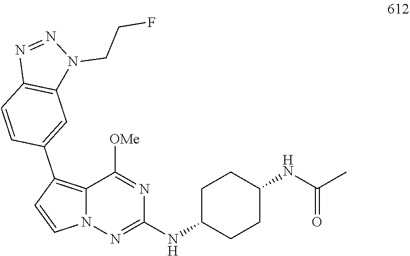 [SMILES: COc1nc(N[C@H]2CC[C@@H](NC(C)=O)CC2)nn2ccc(-c3ccc4nnn(CCF)c4c3)c12]

N-(cis-4-((5-(1-(2-Fluoroethyl)-1H-benzo[d][1,2,3]triazol-6-yl)-4-methoxypyrrolo[2,1-f][1,2,4]triazin-2-yl)amino)cyclohexyl)acetamide 612.

White solid (34 mg, 0.073 mmol, 58.6% yield). ¹H NMR (499 MHz, DMSO-d₆) δ ppm 1.51-1.60 (2H, m), 1.61-1.72 (4H, m), 1.74-1.84 (2H, m), 1.81 (3H, s), 3.68 (2H, dt, J=6.57, 3.29 Hz), 3.95 (3H, s), 4.94 (2H, dt, J=47.15, 4.65 Hz), 5.08 (2H, dt, J=28.00, 4.65 Hz), 6.45 (1H, d, J=6.30 Hz), 6.73 (1H, d, J=2.74 Hz), 7.60-7.63 (1H, m), 7.64 (1H, d, J=2.46 Hz), 7.70 (1H, d, J=7.12 Hz), 8.01 (1H, d, J=10.13 Hz), 8.00 (1H, s); ESIMS found for C₂₃H₂₇FN₈O₂ m/z 467.25 (M+1).

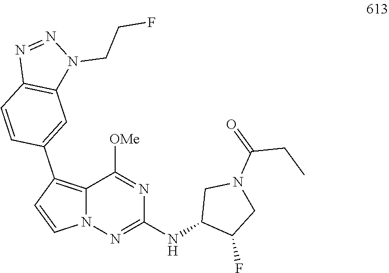 [SMILES: CCC(=O)N1C[C@H](F)[C@H](Nc2nc(OC)c3c(-c4ccc5nnn(CCF)c5c4)ccn3n2)C1]

1-((3S,4R)-3-Fluoro-4-((5-(1-(2-fluoroethyl)-1H-benzo[d][1,2,3]triazol-6-yl)-4-methoxypyrrolo[2,1-f][1,2,4]triazin-2-yl)amino)pyrrolidin-1-yl)propan-1-one 613.

White solid (22 mg, 0.047 mmol, 32.0% yield). ¹H NMR (499 MHz, DMSO-d₆) δ ppm 1.00 (3H, q, J=7.39 Hz), 2.14-2.39 (2H, m), 3.27-3.42 (1H, m), 3.44-3.65 (1H, m), 3.67-3.82 (1H, m), 3.83-3.94 (1H, m), 3.97 (3H, s), 4.27-4.59 (1H, m), 4.94 (2H, dt, J=47.15, 4.65 Hz), 5.08 (2H, dt, J=28.00, 4.65 Hz), 5.25-5.50 (1H, m), 6.79 (1H, t, J=2.05 Hz), 7.09 (1H, dd, J=7.12, 4.93 Hz), 7.63 (1H, d, J=9.58 Hz), 7.68 (1H, dd, J=5.07, 2.60 Hz), 8.00-8.04 (2H, m); ESIMS found for C₂₂H₂₄F₂N₈O₂ m/z 471.2 (M+1).

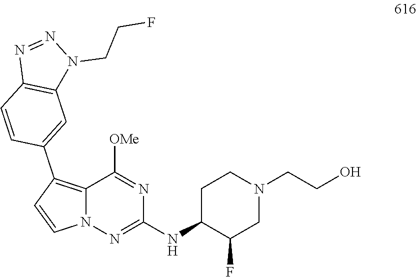 [SMILES: COc1nc(N[C@H]2CCN(CCO)C[C@H]2F)nn2ccc(-c3ccc4nnn(CCF)c4c3)c12]

2-((3R,4S)-3-Fluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-ol 616.

White solid (27 mg, 0.057 mmol, 39.3% yield). ¹H NMR (499 MHz, DMSO-d₆) δ ppm 1.63-1.74 (1H, m), 1.84-1.99 (1H, m), 2.18 (1H, br t, J=10.81 Hz), 2.32 (1H, dd, J=37.85, 12.59 Hz), 2.44 (2H, t, J=6.30 Hz), 2.91 (1H, br d, J=10.68 Hz), 3.11-3.20 (1H, m), 3.50 (2H, t, J=6.30 Hz), 3.71-3.87 (1H, m), 3.95 (3H, s), 4.41 (1H, br s), 4.90 (1H, d, J=49.90 Hz), 4.94 (2H, dt, J=47.20, 4.65 Hz), 5.08 (2H, dt, J=27.95, 4.65 Hz), 6.62 (1H, d, J=7.94 Hz), 6.75 (1H, d, J=2.74 Hz), 7.60-7.63 (1H, m), 7.64 (1H, d, J=2.46 Hz), 7.99-8.03 (2H, m); ESIMS found for C₂₂H₂₆F₂N₈O₂ m/z 473.2 (M+1).

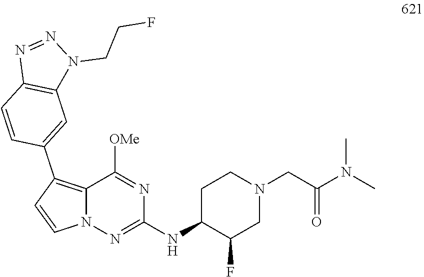 [SMILES: COc1nc(N[C@H]2CCN(CC(=O)N(C)C)C[C@H]2F)nn2ccc(-c3ccc4nnn(CCF)c4c3)c12]

2-((3R,4S)-3-Fluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)-N,N-dimethylacetamide 621.

White solid (6 mg, 0.012 mmol, 15.0% yield). ¹H NMR (499 MHz, DMSO-d₆) δ ppm 1.69 (1H, br dd, J=12.87, 3.56 Hz), 1.86-1.99 (1H, m), 2.31-2.40 (1H, m), 2.51 (1H, dd, J=37.85, 12.35 Hz), 2.81 (3H, s), 2.85 (1H, br d, J=11.23 Hz), 3.02 (3H, s), 3.07-3.14 (1H, m), 3.17-3.26 (2H, m), 3.71-3.87 (1H, m), 3.95 (3H, s), 4.91 (1H, d, J=50.10 Hz), 4.94 (2H, dt, J=47.20, 4.65 Hz), 5.08 (2H, dt, J=28.00, 4.65 Hz), 6.64 (1H, d, J=7.94 Hz), 6.75 (1H, d, J=2.74 Hz), 7.61-7.63 (1H, m), 7.65 (1H, d, J=2.74 Hz), 8.01 (1H, d, J=10.13 Hz), 8.00 (1H, s); ESIMS found for C₂₄H₂₉F₂N₉O₂ m/z 514.25 (M+1).

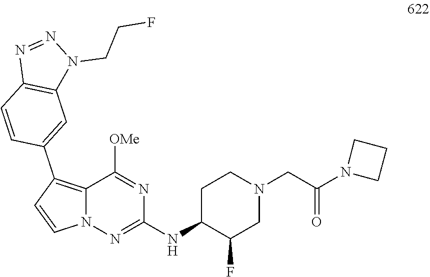 [SMILES: COc1nc(N[C@H]2CCN(CC(=O)N3CCC3)C[C@H]2F)nn2ccc(-c3ccc4nnn(CCF)c4c3)c12]

1-(Azetidin-1-yl)-2-((3R,4S)-3-fluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-one 622.

White solid (5 mg, 0.010 mmol, 11.1% yield). ¹H NMR (499 MHz, DMSO-d₆) δ ppm 1.69 (1H, br dd, J=12.87, 3.29 Hz), 1.93 (1H, qd, J=12.27, 3.70 Hz), 2.15-2.23 (2H, m), 2.29 (1H, br t, J=10.54 Hz), 2.45 (1H, dd, J=38.10, 12.05 Hz), 2.85 (1H, br d, J=11.77 Hz), 3.02 (2H, s), 3.11 (1H, br t, J=10.81 Hz), 3.71-3.83 (1H, m), 3.83-3.89 (2H, m), 3.95 (3H, s), 4.16-4.25 (2H, m), 4.91 (1H, d, J=49.65 Hz), 4.94 (2H, dt, J=47.20, 4.65 Hz), 5.08 (2H, dt, J=27.95, 4.52 Hz), 6.65 (1H, d, J=7.94 Hz), 6.75 (1H, d, J=2.74 Hz), 7.62 (1H, dd, J=8.76, 1.64 Hz), 7.65 (1H, d, J=2.46 Hz), 8.01 (1H, d, J=10.13 Hz), 8.00 (1H, s); ESIMS found for C₂₅H₂₉F₂N₉O₂ m/z 526.3 (M+1).

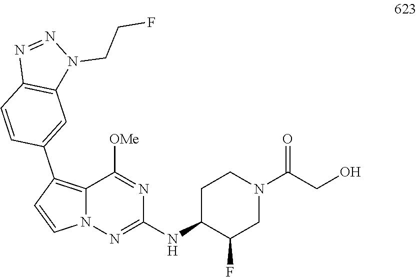 [SMILES: COc1nc(N[C@H]2CCN(C(=O)CO)C[C@H]2F)nn2ccc(-c3ccc4nnn(CCF)c4c3)c12]

1-((3R,4S)-3-Fluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)-2-hydroxyethan-1-one 623.

White solid (12 mg, 0.025 mmol, 44.1% yield). ¹H NMR (499 MHz, DMSO-d₆) δ ppm 1.68-1.91 (2H, m), 2.76-3.06 (1H, m), 3.10-3.19 (1H, m), 3.96 (3H, s), 3.98-4.07 (2H, m), 4.07-4.20 (2H, m), 4.39-4.72 (1H, m), 4.56-4.62 (1H, m), 4.94 (2H, dt, J=47.20, 4.65 Hz), 4.93-5.08 (1H, m), 5.08 (2H, dt, J=27.70, 4.65 Hz), 6.76 (1H, d, J=2.74 Hz), 6.80-6.84 (1H, m), 7.62 (1H, dd, J=8.62, 1.51 Hz), 7.64 (1H, d, J=2.46 Hz), 7.99-8.03 (2H, m); ESIMS found for C₂₂H₂₄F₂N₈O₃ m/z 487.25 (M+1).

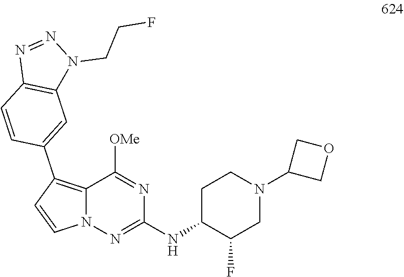 [SMILES: COc1nc(N[C@@H]2CCN(C3COC3)C[C@@H]2F)nn2ccc(-c3ccc4nnn(CCF)c4c3)c12]

N-((3S,4R)-3-Fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-2-amine 624.

White solid (16 mg, 0.033 mmol, 58.7% yield). ¹H NMR (499 MHz, DMSO-d₆) δ ppm 1.69-1.78 (1H, m), 1.92 (1H, qd, J=12.05, 3.01 Hz), 1.98-2.06 (1H, m), 2.16 (1H, dd, J=37.00, 12.59 Hz), 2.76 (1H, br d, J=11.77 Hz), 2.99 (1H, br t, J=10.40 Hz), 3.50 (1H, quin, J=6.23 Hz), 3.75-3.90 (1H, m), 3.96 (3H, s), 4.40 (1H, t, J=6.02 Hz), 4.46 (1H, t, J=6.02 Hz), 4.54 (2H, td, J=6.43, 3.01 Hz), 4.94 (2H, dt, J=47.15, 4.65 Hz), 4.87-5.00 (1H, m), 5.08 (2H, dt, J=27.95, 4.65 Hz), 6.69 (1H, d, J=7.94 Hz), 6.75 (1H, d, J=2.46 Hz), 7.62 (1H, dd, J=8.62, 1.51 Hz), 7.64 (1H, d, J=2.74 Hz), 7.99-8.03 (2H, m); ESIMS found for C₂₃H₂₆F₂N₈O₂ m/z 485.2 (M+1).

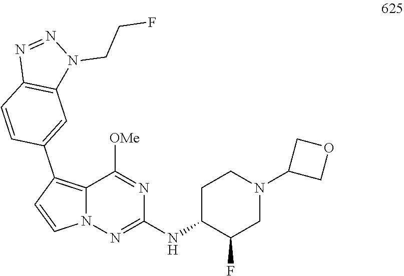 [SMILES: COc1nc(N[C@@H]2CCN(C3COC3)C[C@H]2F)nn2ccc(-c3ccc4nnn(CCF)c4c3)c12]

N-((3R,4R)-3-Fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-2-amine 625.

White solid (22 mg, 0.045 mmol, 55.7% yield). ¹H NMR (499 MHz, DMSO-d₆) δ ppm 1.48-1.59 (1H, m), 1.95 (1H, td, J=11.57, 2.33 Hz), 2.02 (2H, dt, J=9.86, 4.93 Hz), 2.66 (1H, br d, J=11.23 Hz), 3.05 (1H, dt, J=10.47, 5.30 Hz), 3.52 (1H, quin, J=6.30 Hz), 3.80-3.91 (1H, m), 3.95 (3H, s), 4.44 (2H, dt, J=19.51, 6.13 Hz), 4.55 (2H, t, J=6.57 Hz), 4.60 (1H, dtd, J=49.65, 9.31, 9.31, 4.65 Hz), 4.94 (2H, dt, J=47.15, 4.65 Hz), 5.07 (2H, dt, J=28.00, 4.65 Hz), 6.74 (1H, d, J=2.46 Hz), 6.91 (1H, d, J=8.76 Hz), 7.62 (1H, dd, J=8.62, 1.51 Hz), 7.65 (1H, d, J=2.46 Hz), 8.00 (1H, s), 8.01 (1H, d, J=9.03 Hz); ESIMS found for C₂₃H₂₆F₂N₈O₂ m/z 485.25 (M+1).

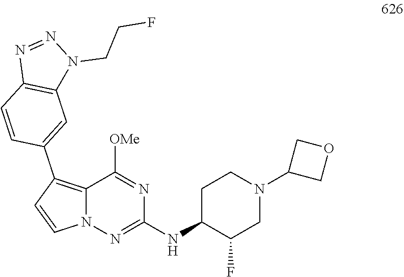 [SMILES: COc1nc(N[C@H]2CCN(C3COC3)C[C@@H]2F)nn2ccc(-c3ccc4nnn(CCF)c4c3)c12]

N-((3S,4S)-3-Fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-2-amine 626.

White solid (12.7 mg, 0.026 mmol, 47.1% yield). ¹H NMR (499 MHz, DMSO-d₆) δ ppm 1.53 (1H, qd, J=11.96, 3.83 Hz), 1.96 (1H, td, J=11.64, 2.46 Hz), 2.02 (2H, dt, J=9.86, 4.93 Hz), 2.66 (1H, br d, J=10.95 Hz), 3.05 (1H, dt, J=10.34, 5.37 Hz), 3.52 (1H, quin, J=6.30 Hz), 3.81-3.92 (1H, m), 3.95 (3H, s), 4.44 (2H, dt, J=19.58, 6.09 Hz), 4.55 (2H, t, J=6.43 Hz), 4.60 (1H, dtd, J=49.60, 9.38, 9.38, 4.79 Hz), 4.94 (2H, dt, J=47.20, 4.65 Hz), 5.08 (2H, dt, J=28.00, 4.65 Hz), 6.74 (1H, d, J=2.74 Hz), 6.91 (1H, d, J=8.76 Hz), 7.62 (1H, dd, J=8.50, 1.35 Hz), 7.65 (1H, d, J=2.74 Hz), 8.01 (1H, d, J=10.40 Hz), 8.00 (1H, s); ESIMS found for C₂₃H₂₆F₂N₈O₂ m/z 485.2 (M+1).

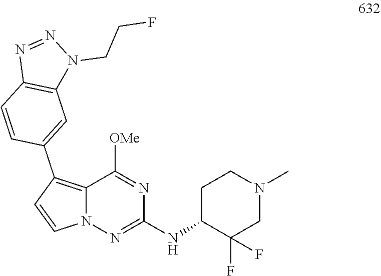 [SMILES: COc1nc(N[C@@H]2CCN(C)CC2(F)F)nn2ccc(-c3ccc4nnn(CCF)c4c3)c12]

(R)—N-(3,3-Difluoro-1-methylpiperidin-4-yl)-5-(1-(2-fluoroethyl)-1H-benzo[d][1,2,3]triazol-6-yl)-4-methoxypyrrolo[2,1-f][1,2,4]triazin-2-amine 632.

White solid (20 mg, 0.043 mmol, 36.0% yield). ¹H NMR (499 MHz, DMSO-d₆) δ ppm 1.77-1.90 (2H, m), 2.12-2.22 (1H, m), 2.26 (3H, s), 2.38 (1H, dd, J=27.15, 11.77 Hz), 2.78 (1H, br d, J=11.77 Hz), 3.05 (1H, td, J=9.79, 8.08 Hz), 3.97 (3H, s), 4.18-4.32 (1H, m), 4.94 (2H, dt, J=47.20, 4.65 Hz), 5.08 (2H, dt, J=28.00, 4.65 Hz), 6.77 (1H, d, J=2.46 Hz), 6.83 (1H, d, J=9.31 Hz), 7.60-7.64 (1H, m), 7.66 (1H, d, J=2.74 Hz), 8.00-8.03 (2H, m); ESIMS found for C₂₁H₂₃F₃N₈O m/z 461.1 (M+1).

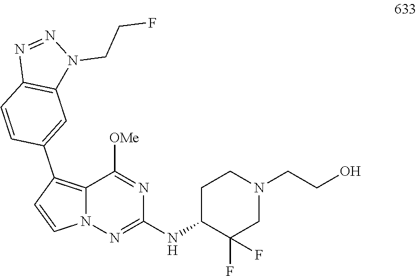

(R)-2-(3,3-Difluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-ol 633.

White solid (11 mg, 0.022 mmol, 16.2% yield). ¹H NMR (499 MHz, DMSO-d₆) δ ppm 1.74-1.82 (1H, m), 1.82-1.89 (1H, m), 2.24-2.34 (1H, m), 2.50-2.54 (3H, m), 2.90 (1H, br d, J=12.05 Hz), 3.13-3.23 (1H, m), 3.52 (2H, br q, J=4.65 Hz), 3.97 (3H, s), 4.18-4.33 (1H, m), 4.49 (1H, t, J=4.65 Hz), 4.94 (2H, dt, J=47.20, 4.65 Hz), 5.08 (2H, dt, J=27.70, 4.65 Hz), 6.77 (1H, d, J=2.74 Hz), 6.83 (1H, d, J=9.31 Hz), 7.61-7.64 (1H, m), 7.66 (1H, d, J=2.46 Hz), 8.00-8.03 (2H, m); ESIMS found for C₂₂H₂₅F₃N₈O₂ m/z 491.2 (M+1).

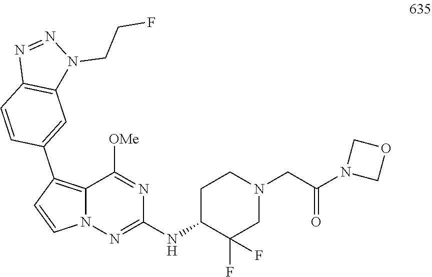 [SMILES: COc1nc(N[C@@H]2CCN(CC(=O)N3COC3)CC2(F)F)nn2ccc(-c3ccc4nnn(CCF)c4c3)c12]

(R)-1-(Azetidin-1-yl)-2-(3,3-difluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-one 635.

White solid (5 mg, 0.009 mmol, 19.1% yield). ¹H NMR (499 MHz, DMSO-d₆) δ ppm 1.76-1.91 (2H, m), 2.19 (2H, quin, J=7.73 Hz), 2.43 (1H, td, J=11.50, 3.01 Hz), 2.68 (1H, ddd, J=27.95, 12.35, 1.35 Hz), 2.85 (1H, br d, J=11.77 Hz), 3.08-3.18 (1H, m), 3.11 (2H, d, J=5.75 Hz), 3.86 (2H, t, J=7.67 Hz), 3.97 (3H, s), 4.17 (2H, t, J=7.67 Hz), 4.22-4.33 (1H, m), 4.94 (2H, dt, J=47.20, 4.65 Hz), 5.08 (2H, dt, J=27.70, 4.65 Hz), 6.77 (1H, d, J=2.46 Hz), 6.85 (1H, d, J=9.31 Hz), 7.60-7.64 (1H, m), 7.67 (1H, d, J=2.46 Hz), 8.00-8.03 (2H, m); ESIMS found for C₂₅H₂₈F₃N₉O₂ m/z 544.25 (M+1).

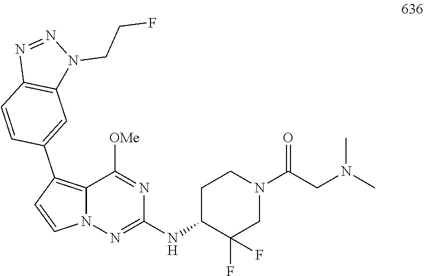

(R)-1-(3,3-Difluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)-2-(dimethylamino)ethan-1-one 636.

White solid (12 mg, 0.023 mmol, 22.7% yield). ¹H NMR (499 MHz, DMSO-d₆) δ ppm 1.58-1.86 (1H, m), 1.88-2.00 (1H, m), 2.19 (6H, s), 2.95-3.09 (2H, m), 3.18-3.26 (1H, m), 3.62-3.81 (1H, m), 3.98 (3H, s), 4.03-4.28 (1H, m), 4.41-4.63 (2H, m), 4.94 (2H, dt, J=46.90, 4.38 Hz), 5.08 (2H, dt, J=27.70, 4.38 Hz), 6.78 (1H, d, J=2.19 Hz), 6.94-7.05 (1H, m), 7.63 (1H, br d, J=9.31 Hz), 7.65 (1H, br s), 7.99-8.04 (2H, m); ESIMS found for C₂₄H₂₈F₃N₉O₂ m/z 532.2 (M+1).

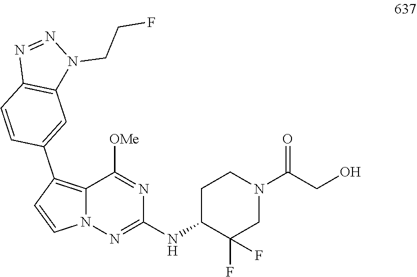 [SMILES: COc1nc(N[C@@H]2CCN(C(=O)CO)CC2(F)F)nn2ccc(-c3ccc4nnn(CCF)c4c3)c12]

(R)-1-(3,3-Difluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)-2-hydroxyethan-1-one 637.

Fluffy white solid (9 mg, 0.018 mmol, 51.6% yield). ¹H NMR (499 MHz, DMSO-d₆) δ ppm 1.59-1.84 (1H, m), 1.93 (1H, br t, J=12.87 Hz), 3.06 (1H, br t, J=12.46 Hz), 3.59-3.84 (1H, m), 3.98 (3H, s), 4.04-4.31 (3H, m), 4.42-4.63 (2H, m), 4.77-4.85 (1H, m), 4.94 (2H, dt, J=47.15, 4.65 Hz), 5.08 (2H, dt, J=28.00, 4.65 Hz), 6.78 (1H, d, J=2.74 Hz), 7.02 (1H, d, J=9.31 Hz), 7.61-7.64 (1H, m), 7.65 (1H, d, J=2.46 Hz), 8.00-8.03 (2H, m); ESIMS found for C₂₂H₂₃F₃N₈O₃ m/z 505.2 (M+1).

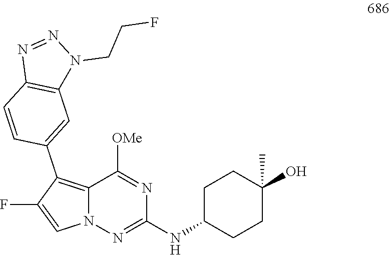 [SMILES: COc1nc(N[C@H]2CC[C@](C)(O)CC2)nn2cc(F)c(-c3ccc4nnn(CCF)c4c3)c12]

(1r,4r)-4-((6-Fluoro-5-(1-(2-fluoroethyl)-1H-benzo[d][1,2,3]triazol-6-yl)-4-methoxypyrrolo[2,1-f][1,2,4]triazin-2-yl)amino)-1-methylcyclohexan-1-ol 686.

Off-white solid (12 mg, 0.026 mmol, 31.9% yield). ¹H NMR (499 MHz, DMSO-d₆) δ ppm 1.14 (3H, s), 1.36-1.53 (4H, m), 1.55-1.66 (2H, m), 1.80-1.92 (2H, m), 3.56-3.68 (1H, m), 3.89 (3H, s), 4.24 (1H, s), 4.92 (2H, dt, J=47.40, 4.65 Hz), 5.08 (2H, dt, J=27.95, 4.65 Hz), 6.59 (1H, d, J=7.94 Hz), 7.54 (1H, d, J=8.76 Hz), 7.84 (1H, d, J=2.74 Hz), 7.96 (1H, s), 8.06 (1H, d, J=8.76 Hz); ESIMS found for C₂₂H₂₅F₂N₇O₂ m/z 458.2 (M+1).

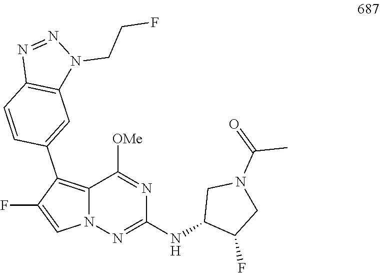 [SMILES: COc1nc(N[C@@H]2CN(C(C)=O)C[C@@H]2F)nn2cc(F)c(-c3ccc4nnn(CCF)c4c3)c12]

1-((3S,4R)-3-Fluoro-4-((6-fluoro-5-(1-(2-fluoroethyl)-1H-benzo[d][1,2,3]triazol-6-yl)-4-methoxypyrrolo[2,1-f][1,2,4]triazin-2-yl)amino)pyrrolidin-1-yl)ethan-1-one 687.

White solid (13.8 mg, 0.029 mmol, 88.6% yield). ¹H NMR (499 MHz, DMSO-d₆) δ ppm 1.93-2.01 (3H, m), 3.45-3.56 (1H, m), 3.57-3.75 (1H, m), 3.81-3.89 (1H, m), 3.90 (3H, s), 3.93 (1H, d, J=1.64 Hz), 4.28-4.57 (1H, m), 4.93 (2H, dt, J=47.45, 4.65 Hz), 5.09 (2H, dt, J=28.00, 4.65 Hz), 5.24-5.48 (1H, m), 7.20 (1H, dd, J=7.39, 1.37 Hz), 7.55 (1H, d, J=8.76 Hz), 7.87 (1H, dd, J=4.38, 3.01 Hz), 7.99 (1H, s), 8.07 (1H, d, J=8.49 Hz); ESIMS found for C₂₁H₂₁F₃N₈O₂ m/z 475.2 (M+1).

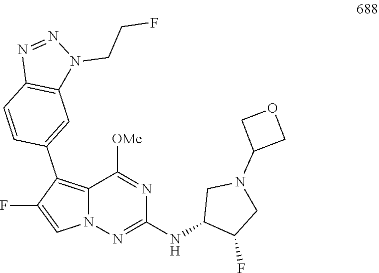 [SMILES: COc1nc(N[C@@H]2CN(C3COC3)C[C@@H]2F)nn2cc(F)c(-c3ccc4nnn(CCF)c4c3)c12]

6-Fluoro-N-((3R,4S)-4-fluoro-1-(oxetan-3-yl)pyrrolidin-3-yl)-5-(1-(2-fluoroethyl)-1H-benzo[d][1,2,3]triazol-6-yl)-4-methoxypyrrolo[2,1-f][1,2,4]triazin-2-amine 688.

White solid (10 mg, 0.021 mmol, 62.5% yield). ¹H NMR (499 MHz, DMSO-d₆) δ ppm 2.69 (1H, br t, J=8.90 Hz), 2.77 (1H, dd, J=28.80, 12.35 Hz), 3.01 (1H, br t, J=8.35 Hz), 3.18 (1H, ddd, J=32.90, 11.36, 3.70 Hz), 3.74-3.84 (1H, m), 3.92 (3H, s), 4.20-4.37 (1H, m), 4.47 (2H, t, J=6.02 Hz), 4.60 (2H, t, J=6.57 Hz), 4.93 (2H, dt, J=47.15, 4.65 Hz), 5.09 (2H, dt, J=28.00, 4.65 Hz), 5.24 (1H, dt, J=56.50, 4.04 Hz), 6.96 (1H, br d, J=7.67 Hz), 7.55 (1H, d, J=8.76 Hz), 7.86 (1H, d, J=3.01 Hz), 7.99 (1H, s), 8.07 (1H, d, J=8.76 Hz); ESIMS found for C₂₂H₂₃F₃N₈O₂ m/z 489.15 (M+1).

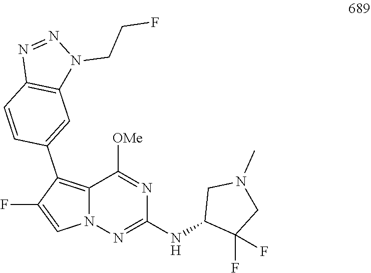 [SMILES: COc1nc(N[C@@H]2CN(C)CC2(F)F)nn2cc(F)c(-c3ccc4nnn(CCF)c4c3)c12]

(R)—N-(4,4-Difluoro-1-methylpyrrolidin-3-yl)-6-fluoro-5-(1-(2-fluoroethyl)-1H-benzo[d][1,2,3]triazol-6-yl)-4-methoxypyrrolo[2,1-f][1,2,4]triazin-2-amine 689.

White solid (15 mg, 0.032 mmol, 75.8% yield). ¹H NMR (499 MHz, DMSO-d₆) δ ppm 2.28 (3H, s), 2.44 (1H, t, J=9.03 Hz), 2.52-2.66 (1H, m), 3.18-3.29 (2H, m), 3.92 (3H, s), 4.49-4.64 (1H, m), 4.92 (2H, dt, J=47.15, 4.65 Hz), 5.09 (2H, dt, J=27.95, 4.65 Hz), 7.12 (1H, d, J=8.76 Hz), 7.55 (1H, d, J=8.21 Hz), 7.88 (1H, d, J=2.74 Hz), 7.99 (1H, s), 8.04-8.10 (1H, m); ESIMS found for C₂₀H₂₀F₄N₈O m/z 465.2 (M+1).

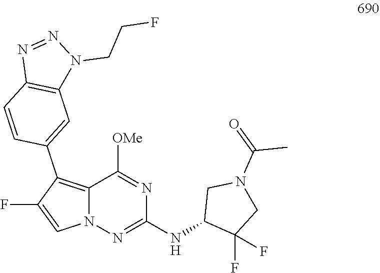 [SMILES: COc1nc(N[C@@H]2CN(C(C)=O)CC2(F)F)nn2cc(F)c(-c3ccc4nnn(CCF)c4c3)c12]

(R)-1-(3,3-Difluoro-4-((6-fluoro-5-(1-(2-fluoroethyl)-1H-benzo[d][1,2,3]triazol-6-yl)-4-methoxypyrrolo[2,1-f][1,2,4]triazin-2-yl)amino)pyrrolidin-1-yl)ethan-1-one 690.

White solid (17 mg, 0.035 mmol, 81.0% yield). ¹H NMR (499 MHz, DMSO-d₆) δ ppm 1.95-1.99 (3H, m), 3.35-3.43 (1H, m), 3.58 (1H, dd, J=10.68, 6.84 Hz), 3.73-3.90 (1H, m), 3.93 (3H, d, J=2.19 Hz), 3.98-4.10 (1H, m), 4.79-4.95 (1H, m), 4.93 (2H, dt, J=47.15, 4.65 Hz), 5.09 (2H, dt, J=27.95, 4.65 Hz), 7.37-7.47 (1H, m), 7.55 (1H, d, J=8.21 Hz), 7.87-7.92 (1H, m), 7.99 (1H, s), 8.07 (1H, d, J=8.21 Hz); ESIMS found for C₂₁H₂₀F₄N₈O₂ m/z 493.2 (M+1).

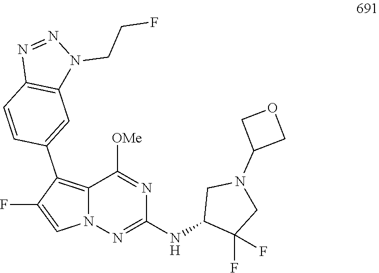 [SMILES: COc1nc(N[C@@H]2CN(C3COC3)CC2(F)F)nn2cc(F)c(-c3ccc4nnn(CCF)c4c3)c12]

(R)—N-(4,4-Difluoro-1-(oxetan-3-yl)pyrrolidin-3-yl)-6-fluoro-5-(1-(2-fluoroethyl)-1H-benzo[d][1,2,3]triazol-6-yl)-4-methoxypyrrolo[2,1-f][1,2,4]triazin-2-amine 691.

White solid (10 mg, 0.020 mmol, 71.1% yield). ¹H NMR (499 MHz, DMSO-d₆) δ ppm 2.51-2.56 (1H, m), 2.67-2.77 (1H, m), 3.25-3.32 (2H, m), 3.70 (1H, quin, J=6.09 Hz), 3.93 (3H, s), 4.46-4.52 (2H, m), 4.55-4.66 (1H, m), 4.59 (2H, q, J=6.84 Hz), 4.93 (2H, dt, J=47.15, 4.65 Hz), 5.09 (2H, dt, J=27.70, 4.65 Hz), 7.14 (1H, d, J=8.76 Hz), 7.55 (1H, d, J=8.76 Hz), 7.88 (1H, d, J=3.01 Hz), 8.00 (1H, s), 8.05-8.10 (1H, m); ESIMS found for C₂₂H₂₂F₄N₈O₂ m/z 507.2 (M+1).

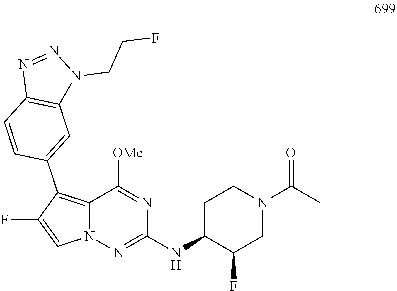 [SMILES: COc1nc(N[C@H]2CCN(C(C)=O)C[C@H]2F)nn2cc(F)c(-c3ccc4nnn(CCF)c4c3)c12]

1-((3R,4S)-3-Fluoro-4-((6-fluoro-5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-one 699.

White solid (21 mg, 0.043 mmol, 91.6% yield). ¹H NMR (499 MHz, DMSO-d₆) δ ppm 1.64-1.73 (1H, m), 1.74-1.91 (1H, m), 1.99-2.06 (3H, m), 2.67-2.98 (1H, m), 3.08-3.26 (1H, m), 3.84-4.00 (1H, m), 3.92 (3H, s), 4.02-4.18 (1H, m), 4.39-4.76 (1H, m), 4.93 (2H, dt, J=47.20, 4.52 Hz), 4.97 (1H, d, J=49.10 Hz), 5.09 (2H, dt, J=27.95, 4.52 Hz), 6.91 (1H, dd, J=7.67, 4.11 Hz), 7.50-7.57 (1H, m), 7.83 (1H, d, J=2.74 Hz), 7.98 (1H, s), 8.07 (1H, d, J=9.31 Hz); ESIMS found for C₂₂H₂₃F₃N₈O₂ m/z 489.15 (M+1).

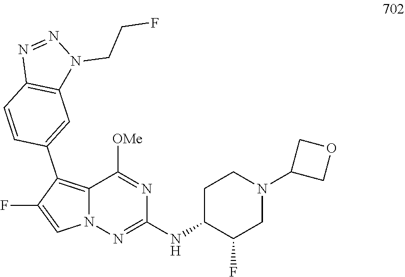 [SMILES: COc1nc(N[C@@H]2CCN(C3COC3)C[C@@H]2F)nn2cc(F)c(-c3ccc4nnn(CCF)c4c3)c12]

6-Fluoro-N-((3S,4R)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-2-amine 702.

White solid (14 mg, 0.028 mmol, 70.7% yield). ¹H NMR (499 MHz, DMSO-d₆) δ ppm 1.66-1.78 (1H, m), 1.85-1.96 (1H, m), 2.01 (1H, br dd, J=5.75, 1.64 Hz), 2.09-2.24 (1H, m), 2.71-2.82 (1H, m), 2.91-3.05 (1H, m), 3.26-3.31 (1H, m), 3.44-3.54 (1H, m), 3.73-3.87 (1H, m), 3.91 (3H, s), 4.35-4.50 (2H, m), 4.51-4.60 (2H, m), 4.93 (3H, dt, J=47.15, 4.65 Hz), 5.09 (2H, dt, J=28.00, 4.65 Hz), 6.78-6.84 (1H, m), 7.54 (1H, d, J=8.76 Hz), 7.82 (1H, d, J=2.46 Hz), 7.97 (1H, s), 8.06 (1H, d, J=8.76 Hz); ESIMS found for C₂₃H₂₅F₃N₈O₂ m/z 503.2 (M+1).

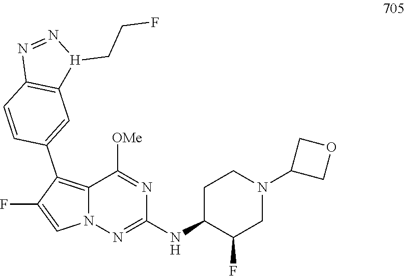

6-Fluoro-N-((3R,4S)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-2-amine 705.

White solid (19 mg, 0.038 mmol, 80.4% yield). ¹H NMR (499 MHz, DMSO-d₆) δ ppm 1.67-1.76 (1H, m), 1.92 (1H, qd, J=12.09, 3.15 Hz), 1.99-2.05 (1H, m), 2.14 (1H, dd, J=36.95, 12.87 Hz), 2.75 (1H, br d, J=11.50 Hz), 2.95-3.05 (1H, m), 3.49 (1H, quin, J=6.37 Hz), 3.71-3.88 (1H, m), 3.91 (3H, s), 4.40 (1H, t, J=6.16 Hz), 4.46 (1H, t, J=6.16 Hz), 4.54 (2H, td, J=6.50, 2.87 Hz), 4.92 (1H, d, J=49.90 Hz), 4.93 (2H, dt, J=47.15, 4.65 Hz), 5.09 (2H, dt, J=28.00, 4.65 Hz), 6.81 (1H, d, J=7.94 Hz), 7.51-7.58 (1H, m), 7.82 (1H, d, J=2.74 Hz), 7.97 (1H, s), 8.03-8.09 (1H, m); ESIMS found for C₂₃H₂₅F₃N₈O₂ m/z 503.25 (M+1).

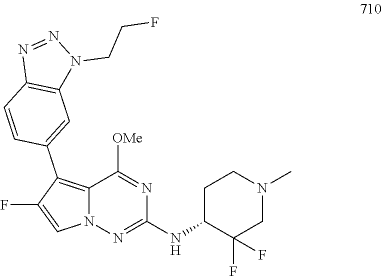 [SMILES: COc1nc(N[C@@H]2CCN(C)CC2(F)F)nn2cc(F)c(-c3ccc4nnn(CCF)c4c3)c12]

(R)—N-(3,3-Difluoro-1-methylpiperidin-4-yl)-6-fluoro-5-(1-(2-fluoroethyl)-1H-benzo[d][1,2,3]triazol-6-yl)-4-methoxypyrrolo[2,1-f][1,2,4]triazin-2-amine 710.

White solid (9 mg, 0.019 mmol, 56.0% yield). ¹H NMR (499 MHz, DMSO-d₆) δ ppm 1.76-1.90 (2H, m), 2.17 (1H, td, J=11.02, 3.15 Hz), 2.25 (3H, s), 2.37 (1H, dd, J=26.85, 12.32 Hz), 2.78 (1H, br d, J=11.77 Hz), 2.98-3.11 (1H, m), 3.92 (3H, s), 4.15-4.29 (1H, m), 4.93 (2H, dt, J=47.15, 4.65 Hz), 5.09 (2H, dt, J=27.95, 4.65 Hz), 6.95 (1H, d, J=9.31 Hz), 7.55 (1H, d, J=8.76 Hz), 7.85 (1H, d, J=2.74 Hz), 7.98 (1H, s), 8.06 (1H, d, J=8.76 Hz); ESIMS found for C₂₁H₂₂F₄N₈O m/z 479.2 (M+1).

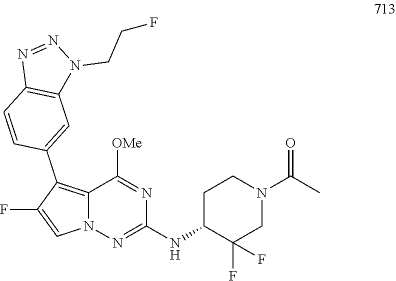 [SMILES: COc1nc(N[C@@H]2CCN(C(C)=O)CC2(F)F)nn2cc(F)c(-c3ccc4nnn(CCF)c4c3)c12]

(R)-1-(3,3-Difluoro-4-((6-fluoro-5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-one 713.

White solid (16 mg, 0.032 mmol, 97.8% yield). ¹H NMR (499 MHz, DMSO-d₆) δ ppm 1.58-1.83 (1H, m), 1.84-1.98 (1H, m), 2.03-2.09 (3H, m), 2.92-3.05 (1H, m), 3.62-3.90 (1H, m), 3.93 (3H, s), 4.11-4.31 (1H, m), 4.43-4.58 (2H, m), 4.93 (2H, dt, J=47.15, 4.65 Hz), 5.09 (2H, dt, J=28.00, 4.65 Hz), 7.11 (1H, dd, J=9.31, 2.19 Hz), 7.55 (1H, d, J=8.76 Hz), 7.80-7.87 (1H, m), 7.99 (1H, s), 8.04-8.11 (1H, m); ESIMS found for C₂₂H₂₂F₄N₈O₂ m/z 507.15 (M+1).

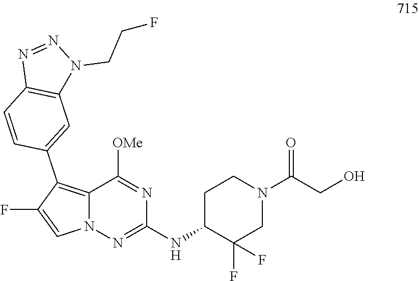 [SMILES: COc1nc(N[C@@H]2CCN(C(=O)CO)CC2(F)F)nn2cc(F)c(-c3ccc4nnn(CCF)c4c3)c12]

(R)-1-(3,3-Difluoro-4-((6-fluoro-5-(1-(2-fluoroethyl)-1H-benzo[d][1,2,3]triazol-6-yl)-4-methoxypyrrolo[2,1-f][1,2,4]triazin-2-yl)amino)piperidin-1-yl)-2-hydroxyethan-1-one 715.

Beige solid (13 mg, 0.025 mmol, 77.0% yield). ¹H NMR (499 MHz, DMSO-d₆) δ ppm 1.60-1.83 (1H, m), 1.85-1.99 (1H, m), 3.04 (1H, brt, J=11.77 Hz), 3.59-3.83 (1H, m), 3.93 (3H, s), 4.05-4.30 (3H, m), 4.44-4.61 (2H, m), 4.79-4.86 (1H, m), 4.93 (2H, dt, J=47.15, 4.65 Hz), 5.09 (2H, dt, J=27.70, 4.65 Hz), 7.13 (1H, d, J=9.31 Hz), 7.55 (1H, d, J=8.49 Hz), 7.84 (1H, d, J=2.74 Hz), 7.99 (1H, s), 8.07 (1H, d, J=8.49 Hz); ESIMS found for C₂₂H₂₂F₄N₈O₃ m/z 523.15 (M+1).

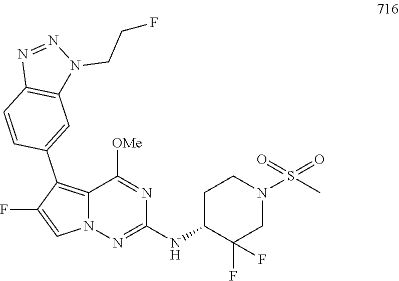 [SMILES: COc1nc(N[C@@H]2CCN(S(C)(=O)=O)CC2(F)F)nn2cc(F)c(-c3ccc4nnn(CCF)c4c3)c12]

(R)—N-(3,3-Difluoro-1-(methylsulfonyl)piperidin-4-yl)-6-fluoro-5-(1-(2-fluoroethyl)-1H-benzo[d][1,2,3]triazol-6-yl)-4-methoxypyrrolo[2,1-f][1,2,4]triazin-2-amine 716.

Off-white solid (9 mg, 0.017 mmol, 51.4% yield). ¹H NMR (499 MHz, DMSO-d₆) δ ppm 1.82-1.94 (1H, m), 1.96-2.05 (1H, m), 2.99 (3H, s), 3.13-3.19 (1H, m), 3.45 (1H, dd, J=26.30, 12.87 Hz), 3.56-3.66 (1H, m), 3.73-3.85 (1H, m), 3.94 (3H, s), 4.41-4.55 (1H, m), 4.93 (2H, dt, J=47.15, 4.65 Hz), 5.09 (2H, dt, J=27.95, 4.65 Hz), 7.17 (1H, d, J=9.31 Hz), 7.55 (1H, d, J=8.76 Hz), 7.83 (1H, d, J=2.74 Hz), 7.99 (1H, s), 8.07 (1H, d, J=8.76 Hz); ESIMS found for C₂₁H₂₂F₄N₈O₃S m/z 543.05 (M+1).

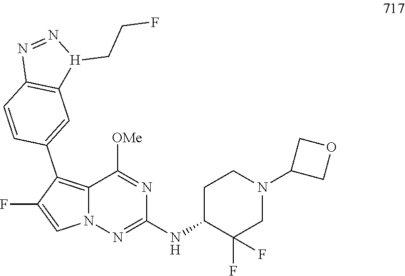

(R)—N-(3,3-Difluoro-1-(oxetan-3-yl)piperidin-4-yl)-6-fluoro-5-(1-(2-fluoroethyl)-1H-benzo[d][1,2,3]triazol-6-yl)-4-methoxypyrrolo[2,1-f][1,2,4]triazin-2-amine 717.

White solid (14 mg, 0.027 mmol, 83.5% yield). ¹H NMR (499 MHz, DMSO-d₆) δ ppm 1.76-1.85 (1H, m), 1.85-1.92 (1H, m), 2.09-2.21 (1H, m), 2.39 (1H, dd, J=27.15, 11.50 Hz), 2.76 (1H, br d, J=11.50 Hz), 2.96-3.07 (1H, m), 3.60 (1H, quin, J=6.23 Hz), 3.88-3.96 (3H, m), 4.19-4.35 (1H, m), 4.44 (2H, dt, J=15.20, 6.23 Hz), 4.55 (2H, td, J=6.64, 3.70 Hz), 4.93 (2H, dt, J=47.20, 4.65 Hz), 5.09 (2H, dt, J=28.00, 4.65 Hz), 7.01 (1H, d, J=9.31 Hz), 7.55 (1H, d, J=8.76 Hz), 7.84 (1H, d, J=2.74 Hz), 7.98 (1H, s), 8.07 (1H, d, J=8.76 Hz); ESIMS found for C₂₃H₂₄F₄N₈O₂ m/z 521.2 (M+1).

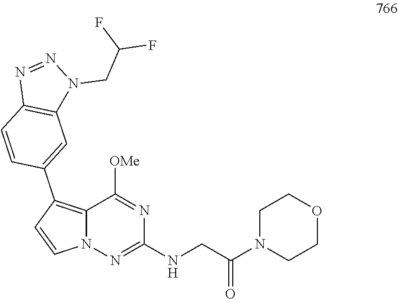 [SMILES: COc1nc(NCC(=O)N2CCOCC2)nn2ccc(-c3ccc4nnn(CC(F)F)c4c3)c12]

2-((5-(1-(2,2-Difluoroethyl)-1H-benzo[d][1,2,3]triazol-6-yl)-4-methoxypyrrolo[2,1-f][1,2,4]triazin-2-yl)amino)-1-morpholinoethan-1-one 766.

White solid (24 mg, 0.051 mmol, 25.5% yield). ¹H NMR (499 MHz, DMSO-d₆) δ ppm 3.48 (2H, br d, J=4.38 Hz), 3.50-3.54 (2H, m), 3.55-3.60 (2H, m), 3.63 (2H, br d, J=4.38 Hz), 3.95 (3H, s), 4.09 (2H, d, J=5.75 Hz), 5.30 (2H, td, J=16.02, 2.74 Hz), 6.60 (1H, tt, J=54.30, 3.00 Hz), 6.50-6.52 (1H, m), 6.76 (1H, d, J=2.46 Hz), 7.63-7.66 (2H, m), 8.03 (1H, s), 8.04 (1H, d, J=6.30 Hz); ESIMS found for C₂₁H₂₂F₂N₈O₃ m/z 473.2 (M+1).

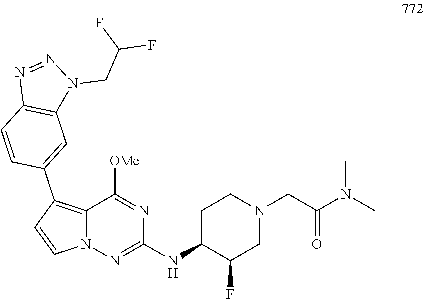 [SMILES: COc1nc(N[C@H]2CCN(CC(=O)N(C)C)C[C@H]2F)nn2ccc(-c3ccc4nnn(CC(F)F)c4c3)c12]

2-((3R,4S)-4-((5-(1-(2,2-Difluoroethyl)-1H-benzo[d][1,2,3]triazol-6-yl)-4-methoxypyrrolo[2,1-f][1,2,4]triazin-2-yl)amino)-3-fluoropiperidin-1-yl)-N,N-dimethylacetamide 772.

White solid (6 mg, 0.011 mmol, 12.4% yield). ¹H NMR (499 MHz, DMSO-d₆) δ ppm 1.64-1.74 (1H, m), 1.87-1.99 (1H, m), 2.31-2.39 (1H, m), 2.51 (1H, dd, J=37.85, 12.60 Hz), 2.81 (3H, s), 2.85 (1H, br d, J=10.68 Hz), 3.02 (3H, s), 3.07-3.15 (1H, m), 3.17-3.26 (2H, m), 3.71-3.88 (1H, m), 3.95 (3H, s), 4.91 (1H, d, J=49.90 Hz), 5.30 (2H, td, J=15.88, 2.74 Hz), 6.60 (2H, tt, J=54.30, 3.00 Hz), 6.65 (1H, d, J=7.67 Hz), 6.75 (1H, d, J=2.46 Hz), 7.63-7.65 (1H, m), 7.66 (1H, d, J=2.46 Hz), 8.01-8.05 (2H, m); ESIMS found for C₂₄H₂₈F₃N₉O₂ m/z 532.2 (M+1).

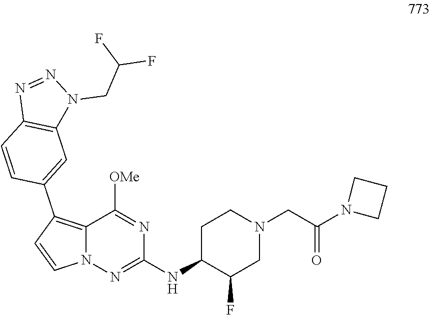 [SMILES: COc1nc(N[C@H]2CCN(CC(=O)N3CCC3)C[C@H]2F)nn2ccc(-c3ccc4nnn(CC(F)F)c4c3)c12]

1-(Azetidin-1-yl)-2-((3R,4S)-4-((5-(1-(2,2-difluoroethyl)-1H-benzo[d][1,2,3]triazol-6-yl)-4-methoxypyrrolo[2,1-f][1,2,4]triazin-2-yl)amino)-3-fluoropiperidin-1-yl)ethan-1-one 773.

White solid (6 mg, 0.011 mmol, 12.9% yield). ¹H NMR (499 MHz, DMSO-d₆) δ ppm 1.64-1.75 (1H, m), 1.93 (1H, qd, J=12.32, 4.11 Hz), 2.14-2.24 (2H, m), 2.26-2.34 (1H, m), 2.45 (1H, dd, J=38.15, 12.32 Hz), 2.85 (1H, br d, J=11.50 Hz), 3.02 (2H, s), 3.11 (1H, br t, J=10.68 Hz), 3.71-3.83 (1H, m), 3.86 (2H, br t, J=7.80 Hz), 3.95 (3H, s), 4.16-4.25 (2H, m), 4.91 (1H, d, J=49.90 Hz), 5.30 (2H, td, J=15.95, 2.87 Hz), 6.60 (1H, tt, J=54.30, 3.01 Hz), 6.66 (1H, d, J=7.67 Hz), 6.75 (1H, d, J=2.46 Hz), 7.63-7.65 (1H, m), 7.66 (1H, d, J=2.74 Hz), 8.01-8.05 (2H, m); ESIMS found for C₂₅H₂₈F₃N₉O₂ m/z 544.25 (M+1).

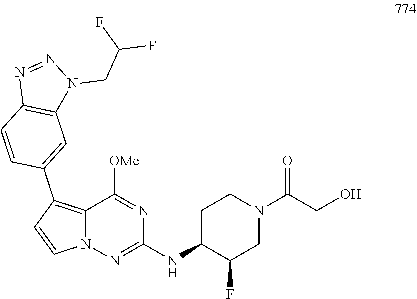 [SMILES: COc1nc(N[C@H]2CCN(C(=O)CO)C[C@H]2F)nn2ccc(-c3ccc4nnn(CC(F)F)c4c3)c12]

1-((3R,4S)-4-((5-(1-(2,2-Difluoroethyl)-1H-benzo[d][1,2,3]triazol-6-yl)-4-methoxypyrrolo[2,1-f][1,2,4]triazin-2-yl)amino)-3-fluoropiperidin-1-yl)-2-hydroxyethan-1-one 774.

White solid (12 mg, 0.024 mmol, 42.6% yield). ¹H NMR (499 MHz, DMSO-d₆) δ ppm 1.67-1.90 (2H, m), 2.75-2.87 (1H, m), 2.92-3.08 (1H, m), 3.96 (3H, s), 3.98-4.21 (4H, m), 4.56-4.74 (2H, m), 4.92-5.10 (1H, m), 5.30 (2H, td, J=15.95, 2.87 Hz), 6.60 (1H, tt, J=54.30, 3.00 Hz), 6.76 (1H, d, J=2.46 Hz), 6.83 (1H, dd, J=7.45, 1.37 Hz), 7.63-7.66 (1H, m), 7.65 (1H, d, J=2.74 Hz), 8.01-8.05 (2H, m); ESIMS found for C₂₂H₂₃F₃N₈O₃ m/z 505.2 (M+1).

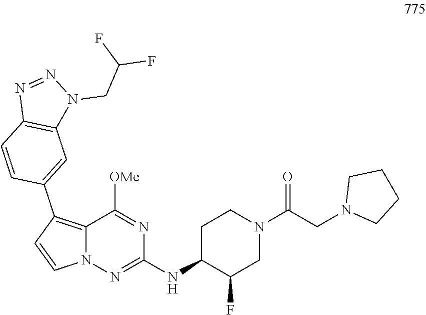 [SMILES: COc1nc(N[C@H]2CCN(C(=O)CN3CCCC3)C[C@H]2F)nn2ccc(-c3ccc4nnn(CC(F)F)c4c3)c12]

1-((3R,4S)-4-((5-(1-(2,2-Difluoroethyl)-1H-benzo[d][1,2,3]triazol-6-yl)-4-methoxypyrrolo[2,1-f][1,2,4]triazin-2-yl)amino)-3-fluoropiperidin-1-yl)-2-(pyrrolidin-1-yl)ethan-1-one 775.

White solid (6 mg, 0.011 mmol, 17.0% yield). ¹H NMR (499 MHz, DMSO-d₆) δ ppm 1.63-1.80 (6H, m), 1.88 (1H, qd, J=12.55, 3.97 Hz), 2.71-2.82 (1H, m), 2.92 (1H, dd, J=39.50, 14.24 Hz), 3.10 (1H, dd, J=27.40, 13.96 Hz), 3.12-3.19 (1H, m), 3.35-3.53 (2H, m), 3.96 (3H, s), 3.98-4.09 (1H, m), 4.14 (1H, br d, J=13.42 Hz), 4.34-4.46 (1H, m), 4.63-4.75 (1H, m), 4.91-5.09 (1H, m), 5.30 (2H, td, J=15.95, 2.60 Hz), 6.61 (1H, tt, J=54.30, 2.87 Hz), 6.75 (1H, d, J=2.46 Hz), 6.80 (1H, dd, J=15.06, 7.67 Hz), 7.59-7.70 (2H, m), 7.99-8.07 (2H, m); ESIMS found for C₂₆H₃₀F₃N₉O₂ m/z 558.3 (M+1).

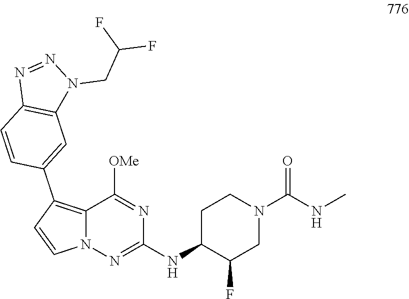 [SMILES: CNC(=O)N1CC[C@H](Nc2nc(OC)c3c(-c4ccc5nnn(CC(F)F)c5c4)ccn3n2)[C@H](F)C1]

(3R,4S)-4-((5-(1-(2,2-Difluoroethyl)-1H-benzo[d][1,2,3]triazol-6-yl)-4-methoxypyrrolo[2,1-f][1,2,4]triazin-2-yl)amino)-3-fluoro-N-methylpiperidine-1-carboxamide 776.

White solid (11 mg, 0.022 mmol, 26.0% yield). ¹H NMR (499 MHz, DMSO-d₆) δ ppm 1.62-1.71 (1H, m), 1.78 (1H, qd, J=12.50, 4.65 Hz), 2.57 (3H, d, J=4.38 Hz), 2.80-2.91 (1H, m), 3.02 (1H, dd, J=38.95, 14.24 Hz), 3.87-3.94 (1H, m), 3.96 (3H, s), 3.98-4.05 (1H, m), 4.25-4.36 (1H, m), 4.92 (1H, d, J=49.65 Hz), 5.30 (2H, td, J=15.95, 2.60 Hz), 6.46 (1H, q, J=4.11 Hz), 6.61 (1H, tt, J=54.30, 3.00 Hz), 6.73-6.77 (2H, m), 7.63-7.66 (1H, m), 7.65 (1H, d, J=2.46 Hz), 8.01-8.06 (2H, m); ESIMS found for C₂₂H₂₄F₃N₉O₂ m/z 504.25 (M+1).

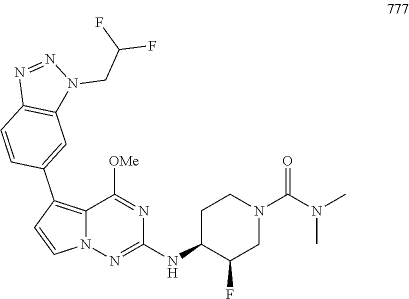 [SMILES: COc1nc(N[C@H]2CCN(C(=O)N(C)C)C[C@H]2F)nn2ccc(-c3ccc4nnn(CC(F)F)c4c3)c12]

(3R,4S)-4-((5-(1-(2,2-Difluoroethyl)-1H-benzo[d][1,2,3]triazol-6-yl)-4-methoxypyrrolo[2,1-f][1,2,4]triazin-2-yl)amino)-3-fluoro-N,N-dimethylpiperidine-1-carboxamide 777.

White solid (5 mg, 0.010 mmol, 11.9% yield). ¹H NMR (499 MHz, DMSO-d₆) δ ppm 1.66-1.77 (1H, m), 1.87-2.00 (1H, m), 2.74 (6H, s), 2.80-2.91 (1H, m), 3.12 (1H, dd, J=38.65, 13.96 Hz), 3.62-3.71 (1H, m), 3.81-3.89 (1H, m), 3.89-4.00 (1H, m), 3.96 (2H, s), 4.95 (1H, d, J=49.35 Hz), 5.30 (2H, td, J=15.95, 2.87 Hz), 6.60 (1H, tt, J=54.30, 3.00 Hz), 6.75 (1H, d, J=2.74 Hz), 6.77 (1H, d, J=7.67 Hz), 7.63-7.66 (1H, m), 7.65 (1H, d, J=2.74 Hz), 8.01-8.05 (2H, m); ESIMS found for C₂₃H₂₆F₃N₉O₂ m/z 518.2 (M+1).

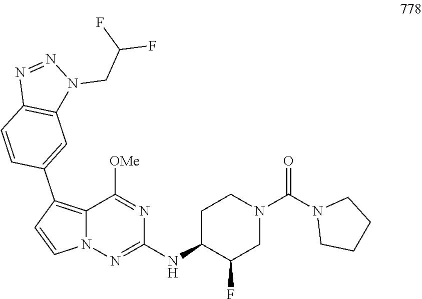 [SMILES: COc1nc(N[C@H]2CCN(C(=O)N3CCCC3)C[C@H]2F)nn2ccc(-c3ccc4nnn(CC(F)F)c4c3)c12]

((3R,4S)-4-((5-(1-(2,2-Difluoroethyl)-1H-benzo[d][1,2,3]triazol-6-yl)-4-methoxypyrrolo[2,1-f][1,2,4]triazin-2-yl)amino)-3-fluoropiperidin-1-yl)(pyrrolidin-1-yl) methanone 778.

White solid (7 mg, 0.013 mmol, 12.2% yield). ¹H NMR (499 MHz, DMSO-d₆) δ ppm 1.67-1.74 (3H, m), 1.75-1.82 (2H, m), 1.91 (1H, qd, J=12.41, 3.83 Hz), 2.79-2.90 (1H, m), 3.10 (1H, dd, J=38.40, 13.96 Hz), 3.20 (2H, dt, J=9.86, 4.93 Hz), 3.34-3.37 (2H, m), 3.77 (1H, br d, J=12.59 Hz), 3.89-4.02 (2H, m), 3.96 (3H, s), 4.95 (1H, d, J=49.10 Hz), 5.30 (2H, td, J=15.95, 2.60 Hz), 6.61 (1H, tt, J=54.30, 3.01 Hz), 6.75 (1H, d, J=2.74 Hz), 6.77 (1H, d, J=7.94 Hz), 7.63-7.67 (2H, m), 8.01-8.06 (2H, m); ESIMS found for C₂₅H₂₈F₃N₉O₂ m/z 544.2 (M+1).

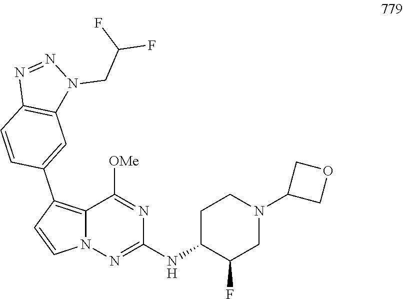 [SMILES: COc1nc(N[C@@H]2CCN(C3COC3)C[C@H]2F)nn2ccc(-c3ccc4nnn(CC(F)F)c4c3)c12]

5-(1-(2,2-Difluoroethyl)-1H-benzo[d][1,2,3]triazol-6-yl)-N-((3R,4R)-3-fluoro-1-(oxetan-3-yl)piperidin-4-yl)-4-methoxypyrrolo[2,1-f][1,2,4]triazin-2-amine 779.

White solid (25 mg, 0.050 mmol, 61.0% yield). ¹H NMR (499 MHz, DMSO-d₆) δ ppm 1.53 (1H, qd, J=12.05, 3.83 Hz), 1.95 (1H, td, J=11.57, 2.33 Hz), 2.02 (2H, dt, J=9.92, 5.03 Hz), 2.66 (1H, br d, J=11.23 Hz), 3.00-3.09 (1H, m), 3.52 (1H, quin, J=6.30 Hz), 3.80-3.92 (1H, m), 3.95 (3H, s), 4.44 (2H, dt, J=19.44, 6.02 Hz), 4.55 (2H, t, J=6.57 Hz), 4.60 (1H, dtd, J=49.35, 9.31, 9.31, 4.65 Hz), 5.30 (2H, td, J=15.88, 2.74 Hz), 6.60 (1H, tt, J=54.30, 3.00 Hz), 6.73 (1H, d, J=2.46 Hz), 6.92 (1H, d, J=8.76 Hz), 7.64 (1H, dd, J=8.75, 1.60 Hz), 7.65 (1H, d, J=2.74 Hz), 8.01-8.05 (2H, m); ESIMS found for C₂₃H₂₅F₃N₈O₂ m/z 503.2 (M+1).

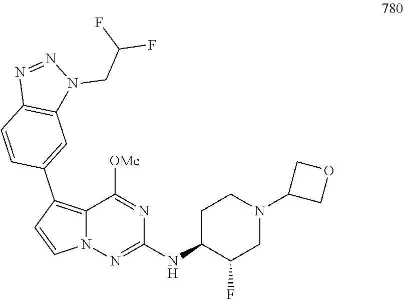 [SMILES: COc1nc(N[C@H]2CCN(C3COC3)C[C@@H]2F)nn2ccc(-c3ccc4nnn(CC(F)F)c4c3)c12]

5-(1-(2,2-Difluoroethyl)-1H-benzo[d][1,2,3]triazol-6-yl)-N-((3S,4S)-3-fluoro-1-(oxetan-3-yl)piperidin-4-yl)-4-methoxypyrrolo[2,1-f][1,2,4]triazin-2-amine 780.

White solid (38.0 mg, 0.076 mmol, 59.8% yield). ¹H NMR (499 MHz, DMSO-d₆) δ ppm 1.53 (1H, qd, J=12.05, 3.83 Hz), 1.96 (1H, td, J=11.57, 2.33 Hz), 2.02 (2H, dt, J=9.86, 4.93 Hz), 2.66 (1H, br d, J=11.22 Hz), 3.05 (1H, dt, J=10.68, 5.34 Hz), 3.52 (1H, quin, J=6.37 Hz), 3.82-3.91 (1H, m), 3.95 (3H, s), 4.44 (2H, dt, J=19.64, 6.06 Hz), 4.55 (2H, t, J=6.57 Hz), 4.60 (1H, dtd, J=49.65, 9.31, 9.31, 4.65 Hz), 5.30 (2H, td, J=15.95, 2.87 Hz), 6.61 (1H, tt, J=54.30, 3.00 Hz), 6.74 (1H, d, J=2.46 Hz), 6.92 (1H, d, J=8.49 Hz), 7.63-7.65 (1H, m), 7.66 (1H, d, J=2.46 Hz), 8.01-8.05 (2H, m); ESIMS found for C₂₃H₂₅F₃N₈O₂ m/z 503.2 (M+1).

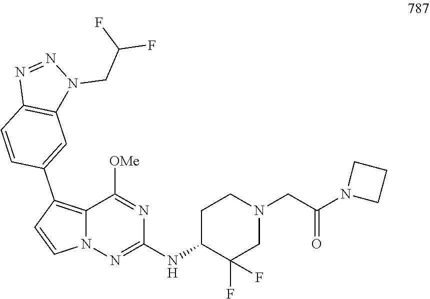 [SMILES: COc1nc(N[C@@H]2CCN(CC(=O)N3CCC3)CC2(F)F)nn2ccc(-c3ccc4nnn(CC(F)F)c4c3)c12]

(R)-1-(Azetidin-1-yl)-2-(4-((5-(1-(2,2-difluoroethyl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one 787.

White solid (8 mg, 0.014 mmol, 29.6% yield). ¹H NMR (499 MHz, DMSO-d₆) δ ppm 1.76-1.92 (2H, m), 2.19 (2H, quin, J=7.67 Hz), 2.38-2.46 (1H, m), 2.68 (1H, dd, J=27.15, 11.77 Hz), 2.85 (1H, br d, J=11.23 Hz), 3.08-3.18 (1H, m), 3.11 (2H, d, J=5.48 Hz), 3.86 (2H, t, J=7.67 Hz), 3.97 (3H, s), 4.17 (2H, t, J=7.80 Hz), 4.22-4.33 (1H, m), 5.30 (2H, td, J=15.95, 2.60 Hz), 6.61 (1H, tt, J=54.30, 2.87 Hz), 6.76 (1H, d, J=2.74 Hz), 6.86 (1H, d, J=9.31 Hz), 7.63-7.66 (1H, m), 7.67 (1H, d, J=2.74 Hz), 8.01-8.05 (2H, m); ESIMS found for C₂₅H₂₇F₄N₉O₂ m/z 562.25 (M+1).

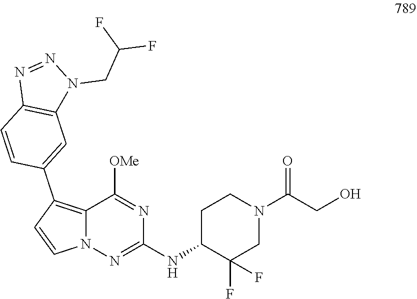 [SMILES: COc1nc(N[C@@H]2CCN(C(=O)CO)CC2(F)F)nn2ccc(-c3ccc4nnn(CC(F)F)c4c3)c12]

(R)-1-(4-((5-(1-(2,2-Difluoroethyl)-1H-benzo[d][1,2,3]triazol-6-yl)-4-methoxypyrrolo[2,1-f][1,2,4]triazin-2-yl)amino)-3,3-difluoropiperidin-1-yl)-2-hydroxyethan-1-one 789.

Fluffy white solid (8 mg, 0.015 mmol, 44.3% yield). ¹H NMR (499 MHz, DMSO-d₆) δ ppm 1.60-1.84 (1H, m), 1.87-2.00 (1H, m), 2.99-3.11 (1H, m), 3.59-3.82 (1H, m), 3.98 (3H, s), 4.04-4.31 (3H, m), 4.43-4.65 (2H, m), 4.77-4.87 (1H, m), 5.30 (2H, td, J=15.81, 2.60 Hz), 6.61 (1H, tt, J=54.30, 3.00 Hz), 6.77 (1H, d, J=2.46 Hz), 7.03 (1H, d, J=9.31 Hz), 7.64-7.67 (2H, m), 8.02-8.06 (2H, m); ESIMS found for C₂₂H₂₂F₄N₈O₃ m/z 523.2 (M+1).

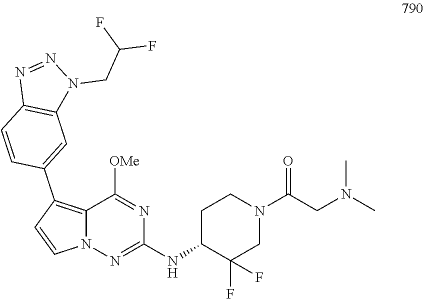 [SMILES: COc1nc(N[C@@H]2CCN(C(=O)CN(C)C)CC2(F)F)nn2ccc(-c3ccc4nnn(CC(F)F)c4c3)c12]

(R)-1-(4-((5-(1-(2,2-Difluoroethyl)-1H-benzo[d][1,2,3]triazol-6-yl)-4-methoxypyrrolo[2,1-f][1,2,4]triazin-2-yl)amino)-3,3-difluoropiperidin-1-yl)-2-(dimethylamino)ethan-1-one 790.

White solid (6 mg, 0.011 mmol, 11.0% yield). ¹H NMR (499 MHz, DMSO-d₆) δ ppm 1.59-1.86 (1H, m), 1.89-1.99 (1H, m), 2.19 (6H, s), 2.95-3.09 (2H, m), 3.17-3.26 (1H, m), 3.62-3.80 (1H, m), 3.98 (3H, s), 4.07-4.31 (1H, m), 4.39-4.62 (2H, m), 5.30 (2H, td, J=15.95, 2.87 Hz), 6.61 (1H, tt, J=54.30, 3.00 Hz), 6.77 (1H, d, J=2.74 Hz), 6.98-7.05 (1H, m), 7.63-7.66 (1H, m), 7.66 (1H, s), 8.02-8.06 (2H, m); ESIMS found for C₂₄H₂₇F₄N₉O₂ m/z 550.15 (M+1).

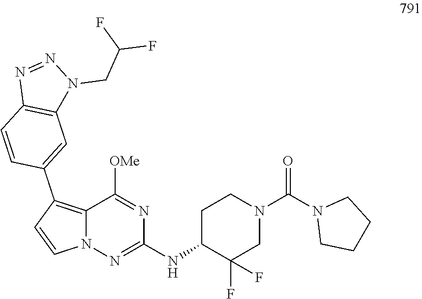 [SMILES: COc1nc(N[C@@H]2CCN(C(=O)N3CCCC3)CC2(F)F)nn2ccc(-c3ccc4nnn(CC(F)F)c4c3)c12]

(R)-(4-((5-(1-(2,2-Difluoroethyl)-1H-benzo[d][1,2,3]triazol-6-yl)-4-methoxypyrrolo[2,1-f][1,2,4]triazin-2-yl)amino)-3,3-difluoropiperidin-1-yl)(pyrrolidin-1-yl)methanone 791.

White solid (12 mg, 0.021 mmol, 31.7% yield). ¹H NMR (499 MHz, DMSO-d₆) δ ppm 1.69-1.80 (4H, m), 1.81-1.88 (1H, m), 1.88-1.94 (1H, m), 2.95-3.07 (1H, m), 3.17-3.27 (2H, m), 3.28-3.33 (2H, m), 3.36-3.39 (1H, m), 3.68 (1H, br d, J=13.14 Hz), 3.82-3.92 (1H, m), 3.97 (3H, s), 4.40-4.56 (1H, m), 5.30 (2H, td, J=15.88, 2.74 Hz), 6.60 (1H, tt, J=54.30, 3.01 Hz), 6.77 (1H, d, J=2.74 Hz), 6.97 (1H, d, J=9.31 Hz), 7.63-7.67 (2H, m), 8.02-8.05 (2H, m); ESIMS found for C₂₅H₂₇F₄N₉O₂ m/z 562.2 (M+1).

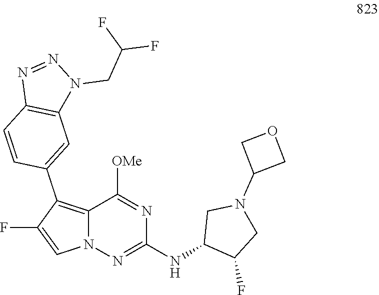 [SMILES: COc1nc(N[C@@H]2CN(C3COC3)C[C@@H]2F)nn2cc(F)c(-c3ccc4nnn(CC(F)F)c4c3)c12]

5-(1-(2,2-Difluoroethyl)-1H-benzo[d][1,2,3]triazol-6-yl)-6-fluoro-N-((3R,4S)-4-fluoro-1-(oxetan-3-yl)pyrrolidin-3-yl)-4-methoxypyrrolo[2,1-f][1,2,4]triazin-2-amine 823.

White solid (7 mg, 0.014 mmol, 28.3% yield). ¹H NMR (499 MHz, DMSO-d₆) δ ppm 2.68 (1H, t, J=9.03 Hz), 2.77 (1H, ddd, J=29.90, 12.05, 1.37 Hz), 3.01 (1H, t, J=8.21 Hz), 3.17 (1H, ddd, J=32.95, 11.91, 4.52 Hz), 3.74-3.84 (1H, m), 3.92 (3H, s), 4.20-4.37 (1H, m), 4.47 (2H, t, J=5.89 Hz), 4.60 (2H, t, J=6.57 Hz), 5.24 (1H, dtd, J=55.65, 4.25, 4.25, 1.40 Hz), 5.32 (2H, td, J=15.88, 3.01 Hz), 6.59 (1H, tt, J=54.30, 3.01 Hz), 6.96 (1H, d, J=7.67 Hz), 7.57 (1H, d, J=8.76 Hz), 7.86 (1H, d, J=3.01 Hz), 8.00 (1H, s), 8.09 (1H, d, J=8.76 Hz); ESIMS found for C₂₂H₂₂F₄N₈O₂ m/z 507.15 (M+1).

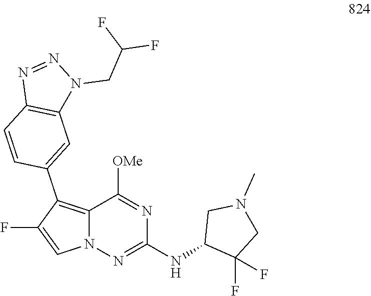 [SMILES: COc1nc(N[C@@H]2CN(C)CC2(F)F)nn2cc(F)c(-c3ccc4nnn(CC(F)F)c4c3)c12]

(R)—N-(4,4-Difluoro-1-methylpyrrolidin-3-yl)-5-(1-(2,2-difluoroethyl)-1H-benzo[d][1,2,3]triazol-6-yl)-6-fluoro-4-methoxypyrrolo[2,1-f][1,2,4]triazin-2-amine 824.

White solid (8.5 mg, 0.018 mmol, 41.3% yield). ¹H NMR (499 MHz, DMSO-d₆) δ ppm 2.28 (3H, s), 2.44 (1H, t, J=9.03 Hz), 2.52-2.62 (1H, m), 3.16-3.28 (2H, m), 3.88-3.97 (3H, m), 4.48-4.64 (1H, m), 5.32 (2H, td, J=15.95, 2.33 Hz), 6.59 (1H, tt, J=54.30, 2.87 Hz), 7.13 (1H, d, J=8.76 Hz), 7.57 (1H, d, J=8.76 Hz), 7.88 (1H, d, J=2.74 Hz), 8.01 (1H, s), 8.09 (1H, d, J=8.49 Hz); ESIMS found for C₂₀H₁₉F₅N₈O m/z 483.2 (M+1).

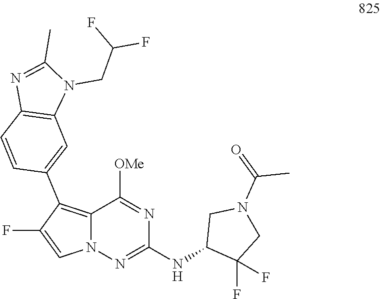 [SMILES: COc1nc(N[C@@H]2CN(C(C)=O)CC2(F)F)nn2cc(F)c(-c3ccc4nc(C)n(CC(F)F)c4c3)c12]

(R)-1-(4-((5-(1-(2,2-Difluoroethyl)-1H-benzo[d][1,2,3]triazol-6-yl)-6-fluoro-4-methoxypyrrolo[2,1-f][1,2,4]triazin-2-yl)amino)-3,3-difluoropyrrolidin-1-yl)ethan-1-one 825.

White solid (12 mg, 0.024 mmol, 55.1% yield). ¹H NMR (499 MHz, DMSO-d₆) δ ppm 1.93-2.02 (3H, m), 3.72-3.90 (2H, m), 3.93 (3H, d, J=2.46 Hz), 4.00-4.12 (2H, m), 4.78-5.00 (1H, m), 5.32 (2H, td, J=15.88, 2.46 Hz), 6.59 (1H, tt, J=54.25, 2.87 Hz), 7.37-7.49 (1H, m), 7.57 (1H, d, J=8.76 Hz), 7.86-7.94 (1H, m), 8.00 (1H, s), 8.09 (1H, d, J=8.76 Hz); ESIMS found for C₂₁H₁₉F₅N₈O₂ m/z 511.1 (M+1).

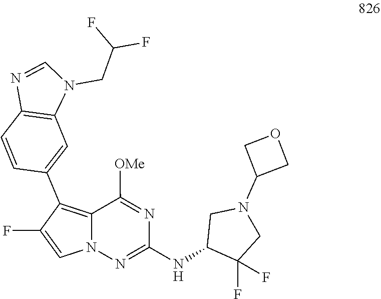 [SMILES: COc1nc(N[C@@H]2CN(C3COC3)CC2(F)F)nn2cc(F)c(-c3ccc4ncn(CC(F)F)c4c3)c12]

(R)—N-(4,4-Difluoro-1-(oxetan-3-yl)pyrrolidin-3-yl)-5-(1-(2,2-difluoroethyl)-1H-benzo[d][1,2,3]triazol-6-yl)-6-fluoro-4-methoxypyrrolo[2,1-f][1,2,4]triazin-2-amine 826.

White solid (2 mg, 0.004 mmol, 8.9% yield). ¹H NMR (499 MHz, DMSO-d₆) δ ppm 2.52-2.56 (1H, m), 2.66-2.78 (1H, m), 3.23-3.30 (2H, m), 3.70 (1H, quin, J=6.09 Hz), 3.92 (3H, s), 4.46-4.52 (2H, m), 4.54-4.62 (2H, m), 4.58-4.66 (1H, m), 5.32 (2H, td, J=16.08, 2.05 Hz), 6.59 (1H, tt, J=54.00, 2.87 Hz), 7.15 (1H, d, J=9.03 Hz), 7.57 (1H, d, J=8.76 Hz), 7.89 (1H, d, J=3.01 Hz), 8.01 (1H, s), 8.09 (1H, d, J=9.31 Hz); ESIMS found for C₂₂H₂₁F₅N₈O₂ m/z 525.2 (M+1).

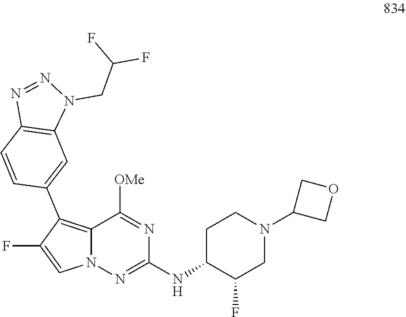 [SMILES: COc1nc(N[C@@H]2CCN(C3COC3)C[C@@H]2F)nn2cc(F)c(-c3ccc4nnn(CC(F)F)c4c3)c12]

5-(1-(2,2-Difluoroethyl)-1H-benzo[d][1,2,3]triazol-6-yl)-6-fluoro-N-((3S,4R)-3-fluoro-1-(oxetan-3-yl)piperidin-4-yl)-4-methoxypyrrolo[2,1-f][1,2,4]triazin-2-amine 834.

White solid (16 mg, 0.031 mmol, 72.0% yield). ¹H NMR (499 MHz, DMSO-d₆) δ ppm 1.66-1.76 (1H, m), 1.92 (1H, qd, J=12.09, 3.42 Hz), 1.98-2.05 (1H, m), 2.15 (1H, dd, J=36.75, 12.59 Hz), 2.76 (1H, br d, J=9.86 Hz), 2.93-3.05 (1H, m), 3.49 (1H, quin, J=6.37 Hz), 3.72-3.88 (1H, m), 3.91 (3H, s), 4.40 (1H, t, J=6.16 Hz), 4.46 (1H, t, J=6.16 Hz), 4.54 (2H, td, J=6.50, 2.87 Hz), 4.92 (1H, d, J=49.40 Hz), 5.32 (2H, td, J=16.02, 2.74 Hz), 6.59 (1H, tt, J=54.30, 3.01 Hz), 6.82 (1H, d, J=7.94 Hz), 7.56 (1H, d, J=8.76 Hz), 7.83 (1H, d, J=3.01 Hz), 7.99 (1H, s), 8.09 (1H, d, J=9.31 Hz); ESIMS found for C₂₃H₂₄F₄N₈O₂ m/z 521.2 (M+1).

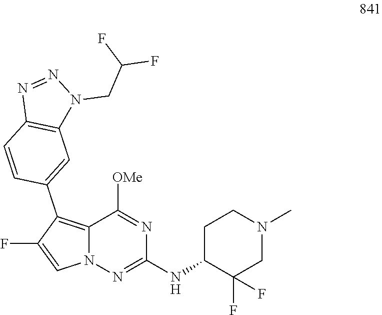 [SMILES: COc1nc(N[C@@H]2CCN(C)CC2(F)F)nn2cc(F)c(-c3ccc4nnn(CC(F)F)c4c3)c12]

(R)—N-(3,3-Difluoro-1-methylpiperidin-4-yl)-5-(1-(2,2-difluoroethyl)-1H-benzo[d][1,2,3]triazol-6-yl)-6-fluoro-4-methoxypyrrolo[2,1-f][1,2,4]triazin-2-amine 841.

White solid (14 mg, 0.028 mmol, 90.7% yield). ¹H NMR (499 MHz, DMSO-d₆) δ ppm 1.73-1.90 (2H, m), 2.17 (1H, td, J=10.88, 3.42 Hz), 2.25 (3H, s), 2.37 (1H, ddd, J=26.30, 12.39, 1.51 Hz), 2.78 (1H, br d, J=11.50 Hz), 2.99-3.11 (1H, m), 3.92 (3H, s), 4.14-4.29 (1H, m), 5.31 (2H, td, J=16.08, 2.87 Hz), 6.59 (1H, tt, J=54.00, 3.01 Hz), 6.96 (1H, d, J=9.31 Hz), 7.57 (1H, d, J=8.49 Hz), 7.85 (1H, d, J=3.01 Hz), 7.99 (1H, s), 8.09 (1H, d, J=8.76 Hz); ESIMS found for C₂₁H₂₁F₅N₈O m/z 497.2 (M+1).

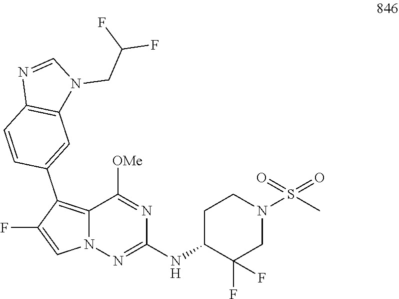 [SMILES: COc1nc(N[C@@H]2CCN(S(C)(=O)=O)CC2(F)F)nn2cc(F)c(-c3ccc4ncn(CC(F)F)c4c3)c12]

(R)—N-(3,3-Difluoro-1-(methylsulfonyl)piperidin-4-yl)-5-(1-(2,2-difluoroethyl)-1H-benzo[d][1,2,3]triazol-6-yl)-6-fluoro-4-methoxypyrrolo[2,1-f][1,2,4]triazin-2-amine 846.

Off-white solid (13 mg, 0.023 mmol, 93.2% yield). ¹H NMR (499 MHz, DMSO-d₆) δ ppm 1.81-1.95 (1H, m), 1.96-2.06 (1H, m), 2.99 (3H, s), 3.11-3.22 (1H, m), 3.38-3.52 (1H, m), 3.57-3.66 (1H, m), 3.73-3.85 (1H, m), 3.93 (3H, s), 4.40-4.57 (1H, m), 5.32 (2H, td, J=16.08, 2.33 Hz), 6.59 (1H, tt, J=54.00, 2.87 Hz), 7.18 (1H, d, J=9.31 Hz), 7.57 (1H, d, J=8.76 Hz), 7.83 (1H, d, J=3.01 Hz), 8.00 (1H, s), 8.09 (1H, d, J=8.76 Hz); ESIMS found for C₂₁H₂₁F₅N₈O₃S m/z 561.1 (M+1).

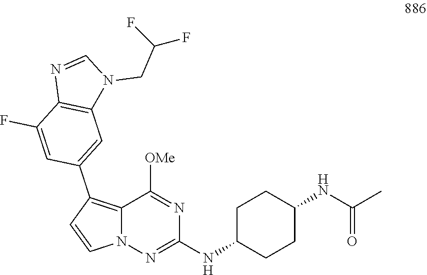 [SMILES: COc1nc(N[C@H]2CC[C@@H](NC(C)=O)CC2)nn2ccc(-c3cc(F)c4ncn(CC(F)F)c4c3)c12]

N-(cis-4-((5-(1-(2,2-Difluoroethyl)-4-fluoro-1H-benzo[d][1,2,3]triazol-6-yl)-4-methoxypyrrolo[2,1-f][1,2,4]triazin-2-yl)amino)cyclohexyl)acetamide 886.

White solid (28.5 mg, 0.057 mmol, 45.6% yield). ¹H NMR (499 MHz, DMSO-d₆) δ ppm 1.51-1.59 (2H, m), 1.60-1.72 (4H, m), 1.74-1.83 (2H, m), 1.81 (3H, s), 3.61-3.74 (2H, m), 3.97 (3H, s), 5.34 (2H, td, J=15.95, 2.60 Hz), 6.51 (1H, d, J=6.30 Hz), 6.62 (1H, tt, J=54.30, 3.00 Hz), 6.78 (1H, d, J=2.46 Hz), 7.51 (1H, dd, J=12.05, 1.10 Hz), 7.65 (1H, d, J=2.74 Hz), 7.70 (1H, d, J=6.84 Hz), 7.90 (1H, s); ESIMS found for C₂₃H₂₅F₃N₈O₂ m/z 503.2 (M+1).

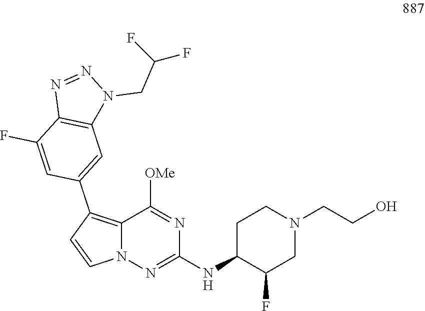 [SMILES: COc1nc(N[C@H]2CCN(CCO)C[C@H]2F)nn2ccc(-c3cc(F)c4nnn(CC(F)F)c4c3)c12]

2-((3R,4S)-4-((5-(1-(2,2-Difluoroethyl)-4-fluoro-1H-benzo[d][1,2,3]triazol-6-yl)-4-methoxypyrrolo[2,1-f][1,2,4]triazin-2-yl)amino)-3-fluoropiperidin-1-yl)ethan-1-ol 887.

White solid (25 mg, 0.049 mmol, 33.8% yield). ¹H NMR (499 MHz, DMSO-d₆) δ ppm 1.63-1.73 (1H, m), 1.91 (1H, qd, J=12.18, 3.70 Hz), 2.13-2.22 (1H, m), 2.32 (1H, dd, J=37.85, 12.32 Hz), 2.44 (2H, t, J=6.30 Hz), 2.91 (1H, br d, J=11.50 Hz), 3.16 (1H, br dd, J=11.36, 9.99 Hz), 3.50 (2H, t, J=6.30 Hz), 3.70-3.87 (1H, m), 3.98 (3H, s), 4.42 (1H, br s), 4.90 (1H, d, J=50.20 Hz), 5.34 (2H, td, J=15.88, 2.74 Hz), 6.62 (1H, tt, J=54.20, 3.00 Hz), 6.68 (1H, d, J=7.94 Hz), 6.80 (1H, d, J=2.74 Hz), 7.51 (1H, dd, J=12.05, 0.82 Hz), 7.66 (1H, d, J=2.74 Hz), 7.91 (1H, s); ESIMS found for C₂₂H₂₄F₄N₈O₂ m/z 509.2 (M+1).

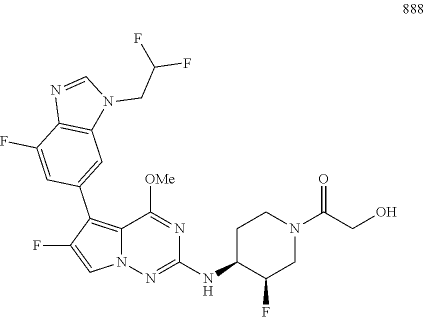 [SMILES: COc1nc(N[C@H]2CCN(C(=O)CO)C[C@H]2F)nn2cc(F)c(-c3cc(F)c4ncn(CC(F)F)c4c3)c12]

1-((3R,4S)-4-((5-(1-(2,2-Difluoroethyl)-4-fluoro-1H-benzo[d][1,2,3]triazol-6-yl)-4-methoxypyrrolo[2,1-f][1,2,4]triazin-2-yl)amino)-3-fluoropiperidin-1-yl)-2-hydroxyethan-1-one 888.

White solid (12 mg, 0.023 mmol, 41.1% yield). ¹H NMR (499 MHz, DMSO-d₆) δ ppm 1.69-1.91 (2H, m), 2.75-2.87 (1H, m), 2.93-3.06 (1H, m), 3.98 (3H, s), 3.99-4.20 (4H, m), 4.42 (1H, br d, J=10.95 Hz), 4.60 (1H, q, J=5.20 Hz), 4.90-5.10 (1H, m), 5.34 (2H, td, J=16.08, 2.60 Hz), 6.62 (1H, tt, J=54.30, 3.00 Hz), 6.81 (1H, d, J=2.74 Hz), 6.85-6.92 (1H, m), 7.48-7.55 (1H, m), 7.66 (1H, d, J=2.46 Hz), 7.91 (1H, s); ESIMS found for C₂₂H₂₂F₄N₈O₃ m/z 523.1 (M+1).

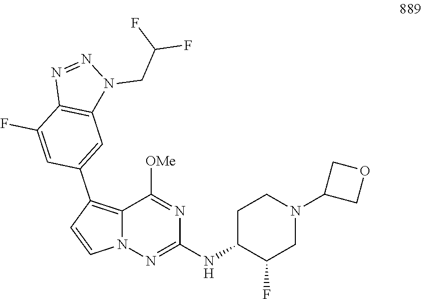 [SMILES: COc1nc(N[C@@H]2CCN(C3COC3)C[C@@H]2F)nn2ccc(-c3cc(F)c4nnn(CC(F)F)c4c3)c12]

5-(1-(2,2-Difluoroethyl)-4-fluoro-1H-benzo[d][1,2,3]triazol-6-yl)-N-((3S,4R)-3-fluoro-1-(oxetan-3-yl)piperidin-4-yl)-4-methoxypyrrolo[2,1-f][1,2,4]triazin-2-amine 889.

White solid (22 mg, 0.042 mmol, 75.2% yield). ¹H NMR (499 MHz, DMSO-d₆) δ ppm 1.67-1.77 (1H, m), 1.92 (1H, qd, J=12.14, 3.29 Hz), 1.98-2.06 (1H, m), 2.16 (1H, dd, J=36.75, 12.59 Hz), 2.76 (1H, br d, J=9.86 Hz), 2.94-3.06 (1H, m), 3.49 (1H, quin, J=6.30 Hz), 3.74-3.90 (1H, m), 3.98 (3H, s), 4.40 (1H, t, J=6.16 Hz), 4.46 (1H, t, J=6.16 Hz), 4.54 (2H, td, J=6.57, 3.01 Hz), 4.94 (1H, d, J=49.35 Hz), 5.34 (2H, td, J=15.88, 2.74 Hz), 6.62 (1H, tt, J=54.30, 3.00 Hz), 6.76 (1H, d, J=7.94 Hz), 6.80 (1H, d, J=2.46 Hz), 7.51 (1H, dd, J=12.05, 1.10 Hz), 7.65 (1H, d, J=2.74 Hz), 7.91 (1H, s); ESIMS found for C₂₃H₂₄F₄N₈O₂ m/z 521.2 (M+1).

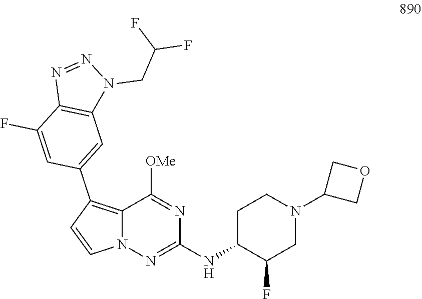 [SMILES: COc1nc(N[C@@H]2CCN(C3COC3)C[C@H]2F)nn2ccc(-c3cc(F)c4nnn(CC(F)F)c4c3)c12]

5-(1-(2,2-Difluoroethyl)-4-fluoro-1H-benzo[d][1,2,3]triazol-6-yl)-N-((3R,4R)-3-fluoro-1-(oxetan-3-yl)piperidin-4-yl)-4-methoxypyrrolo[2,1-f][1,2,4]triazin-2-amine 890.

Off-white solid (29 mg, 0.056 mmol, 68.4% yield). ¹H NMR (499 MHz, DMSO-d₆) δ ppm 1.48-1.59 (1H, m), 1.92-1.98 (1H, m), 2.02 (2H, dt, J=9.72, 4.72 Hz), 2.66 (1H, br d, J=11.50 Hz), 3.05 (1H, dt, J=10.27, 5.00 Hz), 3.52 (1H, quin, J=6.30 Hz), 3.79-3.92 (1H, m), 3.97 (3H, s), 4.44 (2H, dt, J=19.71, 6.16 Hz), 4.55 (2H, t, J=6.57 Hz), 4.60 (1H, dtd, J=49.35, 9.38, 9.38, 4.79 Hz), 5.34 (2H, td, J=15.81, 2.60 Hz), 6.62 (1H, tt, J=54.30, 3.00 Hz), 6.79 (1H, d, J=2.74 Hz), 6.96 (1H, d, J=8.49 Hz), 7.51 (1H, dd, J=12.05, 0.82 Hz), 7.67 (1H, d, J=2.74 Hz), 7.91 (1H, s); ESIMS found for C₂₃H₂₄F₄N₈O₂ m/z 521.2 (M+1).

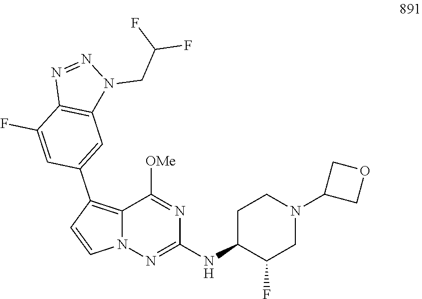 [SMILES: COc1nc(N[C@H]2CCN(C3COC3)C[C@@H]2F)nn2ccc(-c3cc(F)c4nnn(CC(F)F)c4c3)c12]

5-(1-(2,2-Difluoroethyl)-4-fluoro-1H-benzo[d][1,2,3]triazol-6-yl)-N-((3S,4S)-3-fluoro-1-(oxetan-3-yl)piperidin-4-yl)-4-methoxypyrrolo[2,1-f][1,2,4]triazin-2-amine 891.

White solid (13.6 mg, 0.026 mmol, 47.0% yield). ¹H NMR (499 MHz, DMSO-d₆) δ ppm 1.53 (1H, qd, J=12.00, 3.70 Hz), 1.95 (1H, td, J=11.50, 2.19 Hz), 2.01 (2H, td, J=9.79, 5.34 Hz), 2.66 (1H, br d, J=10.95 Hz), 3.05 (1H, dt, J=10.34, 5.37 Hz), 3.52 (1H, quin, J=6.30 Hz), 3.80-3.92 (1H, m), 3.98 (3H, s), 4.44 (2H, dt, J=19.71, 6.16 Hz), 4.55 (2H, t, J=6.57 Hz), 4.60 (1H, dtd, J=49.30, 9.24, 9.24, 4.79 Hz), 5.34 (2H, td, J=15.95, 2.60 Hz), 6.62 (1H, tt, J=54.30, 3.00 Hz), 6.79 (1H, d, J=2.46 Hz), 6.96 (1H, d, J=8.76 Hz), 7.51 (1H, dd, J=12.05, 0.82 Hz), 7.67 (1H, d, J=2.74 Hz), 7.91 (1H, s); ESIMS found for C₂₃H₂₄F₄N₈O₂ m/z 521.2 (M+1).

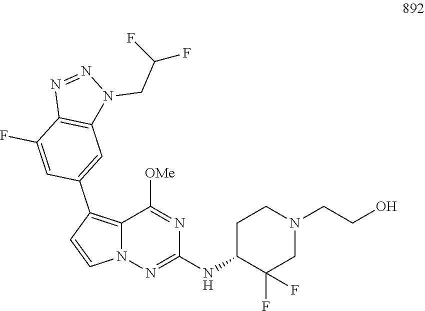 [SMILES: COc1nc(N[C@@H]2CCN(CCO)CC2(F)F)nn2ccc(-c3cc(F)c4nnn(CC(F)F)c4c3)c12]

(R)-2-(4-((5-(1-(2,2-Difluoroethyl)-4-fluoro-1H-benzo[d][1,2,3]triazol-6-yl)-4-methoxypyrrolo[2,1-f][1,2,4]triazin-2-yl)amino)-3,3-difluoropiperidin-1-yl)ethan-1-ol 892.

White solid (9 mg, 0.017 mmol, 12.4% yield). ¹H NMR (499 MHz, DMSO-d₆) δ ppm 1.72-1.89 (2H, m), 2.29 (1H, br t, J=10.68 Hz), 2.50-2.54 (3H, m), 2.91 (1H, br d, J=10.68 Hz), 3.11-3.23 (1H, m), 3.52 (2H, q, J=5.84 Hz), 3.99 (3H, s), 4.16-4.33 (1H, m), 4.49 (1H, t, J=5.20 Hz), 5.34 (2H, td, J=15.88, 2.74 Hz), 6.62 (1H, tt, J=54.15, 3.00 Hz), 6.81 (1H, d, J=2.46 Hz), 6.89 (1H, d, J=9.31 Hz), 7.52 (1H, dd, J=12.18, 0.96 Hz), 7.67 (1H, d, J=2.74 Hz), 7.91 (1H, s); ESIMS found for C₂₂H₂₃F₅N₈O₂ m/z 527.2 (M+1).

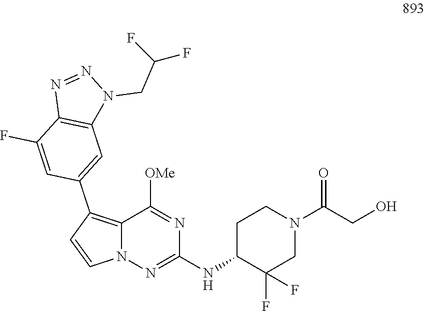 [SMILES: COc1nc(N[C@@H]2CCN(C(=O)CO)CC2(F)F)nn2ccc(-c3cc(F)c4nnn(CC(F)F)c4c3)c12]

(R)-1-(4-((5-(1-(2,2-Difluoroethyl)-4-fluoro-1H-benzo[d][1,2,3]triazol-6-yl)-4-methoxypyrrolo[2,1-f][1,2,4]triazin-2-yl)amino)-3,3-difluoropiperidin-1-yl)-2-hydroxyethan-1-one 893.

Fluffy white solid (9 mg, 0.017 mmol, 48.1% yield). ¹H NMR (499 MHz, DMSO-d₆) δ ppm 1.61-1.84 (1H, m), 1.87-2.00 (1H, m), 3.00-3.13 (1H, m), 3.59-3.86 (1H, m), 4.00 (3H, s), 4.05-4.31 (3H, m), 4.43-4.64 (2H, m), 4.82 (1H, br d, J=4.38 Hz), 5.34 (2H, td, J=15.95, 2.87 Hz), 6.62 (1H, tt, J=54.20, 3.00 Hz), 6.83 (1H, d, J=2.46 Hz), 7.08 (1H, d, J=9.31 Hz), 7.52 (1H, dd, J=12.05, 1.10 Hz), 7.66 (1H, d, J=2.46 Hz), 7.92 (1H, s); ESIMS found for C₂₂H₂₁F₅N₈O₃ m/z 541.25 (M+1).

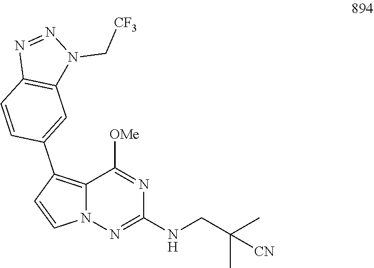 [SMILES: COc1nc(NCC(C)(C)C#N)nn2ccc(-c3ccc4nnn(CC(F)(F)F)c4c3)c12]

3-((4-Methoxy-5-(1-(2,2,2-trifluoroethyl)-1H-benzo[d][1,2,3]triazol-6-yl)pyrrolo[2,1-f][1,2,4]triazin-2-yl)amino)-2,2-dimethylpropanenitrile 894.

White solid (40 mg, 0.090 mmol, 83.9% yield). ¹H NMR (499 MHz, DMSO-d₆) δ ppm 1.36 (6H, s), 3.49 (2H, d, J=6.84 Hz), 3.97 (3H, s), 5.88 (2H, q, J=9.03 Hz), 6.76 (1H, d, J=2.46 Hz), 7.15 (1H, t, J=6.71 Hz), 7.68 (1H, dd, J=8.80, 1.35 Hz), 7.67 (1H, d, J=2.74 Hz), 8.08 (1H, d, J=8.76 Hz), 8.11 (1H, s); ESIMS found for C₂₀H₁₉F₃N₈O m/z 445.2 (M+1).

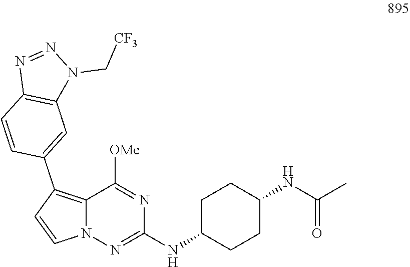 [SMILES: COc1nc(N[C@H]2CC[C@@H](NC(C)=O)CC2)nn2ccc(-c3ccc4nnn(CC(F)(F)F)c4c3)c12]

N-(cis-4-((4-Methoxy-5-(1-(2,2,2-trifluoroethyl)-1H-benzo[d][1,2,3]triazol-6-yl)pyrrolo[2,1-f][1,2,4]triazin-2-yl)amino)cyclohexyl)acetamide 895.

White solid (29 mg, 0.058 mmol, 46.4% yield). ¹H NMR (499 MHz, DMSO-d₆) δ ppm 1.50-1.60 (2H, m), 1.60-1.72 (4H, m), 1.74-1.83 (2H, m), 1.81 (3H, s), 3.68 (2H, br dd, J=6.57, 3.56 Hz), 3.94 (3H, s), 5.87 (2H, q, J=9.22 Hz), 6.48 (1H, d, J=6.30 Hz), 6.72 (1H, d, J=2.46 Hz), 7.65 (1H, d, J=2.74 Hz), 7.67 (1H, dd, J=8.62, 1.51 Hz), 7.70 (1H, d, J=6.84 Hz), 8.07 (1H, d, J=8.49 Hz), 8.10 (1H, s); ESIMS found for C₂₃H₂₅F₃N₈O₂ m/z 503.2 (M+1).

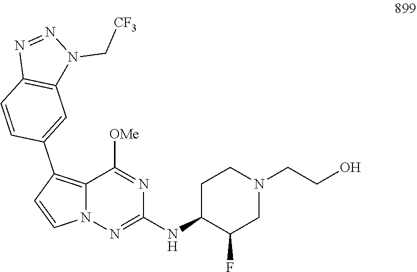 [SMILES: COc1nc(N[C@H]2CCN(CCO)C[C@H]2F)nn2ccc(-c3ccc4nnn(CC(F)(F)F)c4c3)c12]

2-((3R,4S)-3-Fluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-ol 899.

White solid (28 mg, 0.055 mmol, 37.9% yield). ¹H NMR (499 MHz, DMSO-d₆) δ ppm 1.62-1.74 (1H, m), 1.91 (1H, qd, J=12.18, 3.70 Hz), 2.12-2.22 (1H, m), 2.32 (1H, dd, J=37.85, 12.32 Hz), 2.44 (2H, t, J=6.30 Hz), 2.86-2.96 (1H, m), 3.16 (1H, br dd, J=11.36, 9.99 Hz), 3.51 (2H, t, J=6.30 Hz), 3.72-3.87 (1H, m), 3.94 (3H, s), 4.42 (1H, br s), 4.90 (1H, d, J=49.65 Hz), 5.87 (2H, q, J=9.22 Hz), 6.65 (1H, d, J=7.94 Hz), 6.74 (1H, d, J=2.74 Hz), 7.66 (1H, d, J=2.46 Hz), 7.67 (1H, dd, J=8.76, 1.37 Hz), 8.07 (1H, d, J=8.76 Hz), 8.10 (1H, s); ESIMS found for C₂₂H₂₄F₄N₈O₂ m/z 509.2 (M+1).

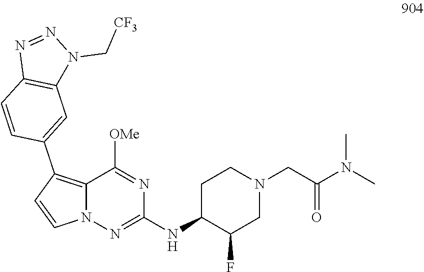 [SMILES: COc1nc(N[C@H]2CCN(CC(=O)N(C)C)C[C@H]2F)nn2ccc(-c3ccc4nnn(CC(F)(F)F)c4c3)c12]

2-((3R,4S)-3-Fluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)-N,N-dimethylacetamide 904.

White solid (10 mg, 0.018 mmol, 20.0% yield). ¹H NMR (499 MHz, DMSO-d₆) δ ppm 1.69 (1H, br dd, J=12.73, 3.42 Hz), 1.86-1.99 (1H, m), 2.30-2.40 (1H, m), 2.51 (1H, dd, J=37.60, 12.32 Hz), 2.81 (3H, s), 2.85 (1H, br dd, J=13.14, 1.64 Hz), 3.02 (3H, s), 3.07-3.15 (1H, m), 3.17-3.26 (2H, m), 3.71-3.87 (1H, m), 3.94 (3H, s), 4.91 (1H, d, J=49.90 Hz), 5.87 (2H, q, J=9.22 Hz), 6.67 (1H, d, J=7.94 Hz), 6.74 (1H, d, J=2.74 Hz), 7.67 (1H, d, J=2.46 Hz), 7.66-7.69 (1H, m), 8.07 (1H, d, J=8.49 Hz), 8.10 (1H, s); ESIMS found for C₂₄H₂₇F₄N₉O₂ m/z 550.25 (M+1).

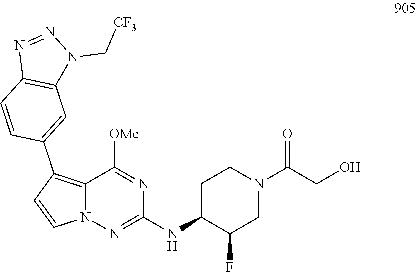 [SMILES: COc1nc(N[C@H]2CCN(C(=O)CO)C[C@H]2F)nn2ccc(-c3ccc4nnn(CC(F)(F)F)c4c3)c12]

1-((3R,4S)-3-Fluoro-4-((4-methoxy-5-(1-(2,2,2-trifluoroethyl)-1H-benzo[d][1,2,3]triazol-6-yl)pyrrolo [2,1-f][1,2,4]triazin-2-yl)amino)piperidin-1l-yl)-2-hydroxyethan-1-one 905.

White solid (13 mg, 0.025 mmol, 44.5% yield). ¹H NMR (499 MHz, DMSO-d₆) δ ppm 1.67-1.92 (2H, m), 2.75-2.89 (1H, m), 2.92-3.08 (1H, m), 3.95 (3H, s), 3.98-4.22 (4H, m), 4.51-4.74 (2H, m), 4.90-5.11 (1H, m), 5.87 (2H, q, J=9.31 Hz), 6.76 (1H, d, J=2.46 Hz), 6.81-6.88 (1H, m), 7.66 (1H, d, J=2.74 Hz), 7.66-7.69 (1H, m), 8.08 (1H, d, J=8.76 Hz), 8.10 (1H, s); ESIMS found for C₂₂H₂₂F₄N₈O₃ m/z 523.15 (M+1).

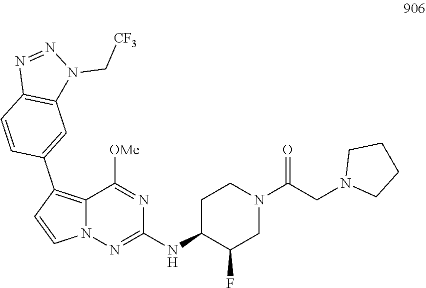 [SMILES: COc1nc(N[C@H]2CCN(C(=O)CN3CCCC3)C[C@H]2F)nn2ccc(-c3ccc4nnn(CC(F)(F)F)c4c3)c12]

1-((3R,4S)-3-Fluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-(pyrrolidin-1-yl)ethan-1-one 906.

White solid (6 mg, 0.010 mmol, 16.5% yield). ¹H NMR (499 MHz, DMSO-d₆) δ ppm 1.61-1.79 (6H, m), 1.88 (1H, qd, J=12.59, 4.38 Hz), 2.69-2.81 (1H, m), 2.92 (1H, dd, J=39.80, 13.96 Hz), 3.09 (1H, dd, J=27.15, 13.69 Hz), 3.13-3.19 (1H, m), 3.35-3.50 (2H, m), 3.95 (3H, s), 3.97-4.09 (1H, m), 4.15 (1H, br d, J=13.96 Hz), 4.35-4.47 (1H, m), 4.61-4.76 (1H, m), 4.90-5.07 (1H, m), 5.88 (2H, q, J=9.03 Hz), 6.75 (1H, d, J=2.46 Hz), 6.82 (1H, dd, J=15.19, 7.80 Hz), 7.66 (1H, d, J=2.46 Hz), 7.68 (1H, dd, J=8.76, 1.37 Hz), 8.08 (1H, d, J=8.76 Hz), 8.10 (1H, s); ESIMS found for C₂H₂₉F₄N₉O₂ m/z 576.3 (M+1).

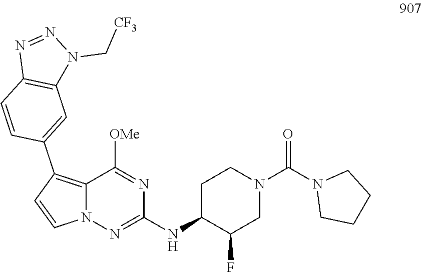 [SMILES: COc1nc(N[C@H]2CCN(C(=O)N3CCCC3)C[C@H]2F)nn2ccc(-c3ccc4nnn(CC(F)(F)F)c4c3)c12]

((3R,4S)-3-Fluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)(pyrrolidin-1-yl) methanone 907.

White solid (5 mg, 0.009 mmol, 8.4% yield). ¹H NMR (499 MHz, DMSO-d₆) δ ppm 1.65-1.74 (3H, m), 1.75-1.82 (2H, m), 1.89 (1H, qd, J=12.41, 4.11 Hz), 2.78-2.87 (1H, m), 3.10 (1H, dd, J=38.40, 13.69 Hz), 3.16-3.23 (2H, m), 3.28-3.37 (2H, m), 3.71-3.82 (1H, m), 3.88-4.01 (2H, m), 3.94 (3H, s), 4.94 (1H, d, J=49.65 Hz), 5.86 (2H, q, J=9.22 Hz), 6.75 (1H, d, J=2.74 Hz), 6.77 (1H, d, J=7.67 Hz), 7.65 (1H, d, J=2.46 Hz), 7.67 (1H, dd, J=8.62, 1.51 Hz), 8.07 (1H, d, J=8.76 Hz), 8.09 (1H, s); ESIMS found for C₂₅H₂₇F₄N₉O₂ m/z 562.25 (M+1).

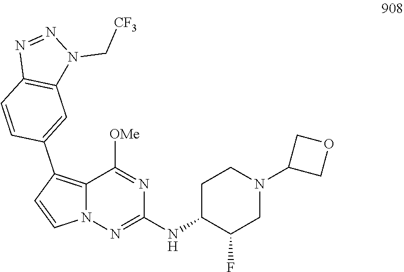 [SMILES: COc1nc(N[C@@H]2CCN(C3COC3)C[C@@H]2F)nn2ccc(-c3ccc4nnn(CC(F)(F)F)c4c3)c12]

N-((3S,4R)-3-Fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine 908.

Off-white solid (21 mg, 0.040 mmol, 71.8% yield). ¹H NMR (499 MHz, DMSO-d₆) δ ppm 1.68-1.78 (1H, m), 1.92 (1H, qd, J=12.05, 3.29 Hz), 1.98-2.06 (1H, m), 2.16 (1H, dd, J=37.30, 12.87 Hz), 2.71-2.82 (1H, m), 2.93-3.06 (1H, m), 3.50 (1H, quin, J=6.37 Hz), 3.74-3.89 (1H, m), 3.95 (3H, s), 4.40 (1H, t, J=6.16 Hz), 4.46 (1H, t, J=6.16 Hz), 4.54 (2H, td, J=6.43, 3.01 Hz), 4.94 (1H, d, J=49.65 Hz), 5.87 (2H, q, J=9.13 Hz), 6.72 (1H, d, J=7.94 Hz), 6.74 (1H, d, J=2.74 Hz), 7.65 (1H, d, J=2.74 Hz), 7.67 (1H, dd, J=8.76, 1.37 Hz), 8.07 (1H, d, J=8.49 Hz), 8.10 (1H, s); ESIMS found for C₂₃H₂₄F₄N₈O₂ m/z 521.2 (M+1).

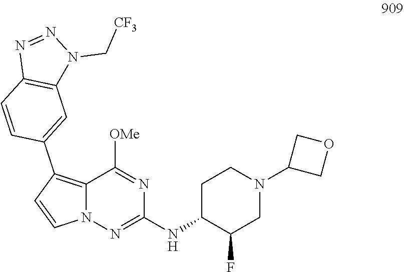 [SMILES: COc1nc(N[C@@H]2CCN(C3COC3)C[C@H]2F)nn2ccc(-c3ccc4nnn(CC(F)(F)F)c4c3)c12]

N-((3R,4R)-3-Fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine 909.

White solid (25 mg, 0.048 mmol, 58.9% yield). ¹H NMR (499 MHz, DMSO-d₆) δ ppm 1.53 (1H, qd, J=11.96, 3.29 Hz), 1.96 (1H, td, J=11.57, 2.33 Hz), 2.02 (2H, dt, J=9.99, 5.13 Hz), 2.61-2.73 (1H, m), 3.05 (1H, dt, J=10.27, 5.27 Hz), 3.52 (1H, quin, J=6.30 Hz), 3.79-3.91 (1H, m), 3.94 (3H, s), 4.44 (2H, dt, J=19.58, 6.23 Hz), 4.55 (2H, t, J=6.57 Hz), 4.60 (1H, dtd, J=49.65, 9.38, 9.38, 4.79 Hz), 5.87 (2H, q, J=9.22 Hz), 6.73 (1H, d, J=2.74 Hz), 6.94 (1H, d, J=8.76 Hz), 7.66-7.69 (1H, m), 7.67 (1H, d, J=2.46 Hz), 8.07 (1H, d, J=8.76 Hz), 8.10 (1H, s); ESIMS found for C₂₃H₂₄F₄N₈O₂ m/z 521.2 (M+1).

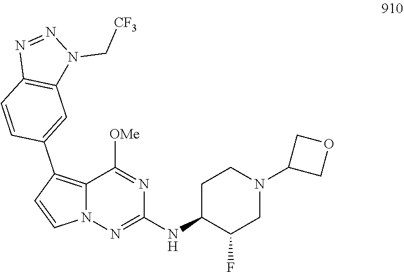 [SMILES: COc1nc(N[C@H]2CCN(C3COC3)C[C@@H]2F)nn2ccc(-c3ccc4nnn(CC(F)(F)F)c4c3)c12]

N-((3S,4S)-3-Fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine 910.

White solid (6 mg, 0.012 mmol, 20.7% yield). ¹H NMR (499 MHz, DMSO-d₆) δ ppm 1.53 (1H, qd, J=11.91, 3.42 Hz), 1.92-1.98 (1H, m), 2.02 (2H, dt, J=10.06, 4.96 Hz), 2.67 (1H, br d, J=10.68 Hz), 2.99-3.11 (1H, m), 3.52 (1H, quin, J=6.30 Hz), 3.82-3.91 (1H, m), 3.94 (3H, s), 4.44 (2H, dt, J=19.58, 6.23 Hz), 4.55 (2H, t, J=6.57 Hz), 4.60 (1H, dtd, J=49.60, 9.38, 9.38, 4.79 Hz), 5.87 (2H, q, J=9.22 Hz), 6.73 (1H, d, J=2.74 Hz), 6.93 (1H, d, J=8.76 Hz), 7.66-7.69 (1H, m), 7.67 (1H, d, J=2.46 Hz), 8.07 (1H, d, J=8.76 Hz), 8.10 (1H, s); ESIMS found for C₂₃H₂₄F₄N₈O₂ m/z 521.2 (M+1).

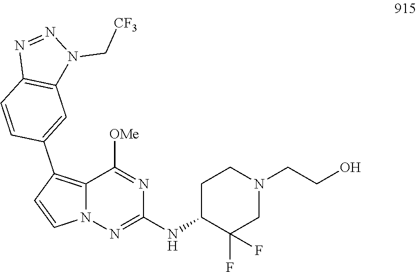

(R)-2-(3,3-Difluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-ol915.

White solid (10 mg, 0.019 mmol, 13.7% yield). ¹H NMR (499 MHz, DMSO-d₆) δ ppm 1.73-1.90 (2H, m), 2.23-2.36 (1H, m), 2.50-2.54 (3H, m), 2.90 (1H, br d, J=11.77 Hz), 3.13-3.23 (1H, m), 3.52 (2H, q, J=5.93 Hz), 3.96 (3H, s), 4.17-4.34 (1H, m), 4.49 (1H, t, J=5.20 Hz), 5.87 (2H, q, J=8.94 Hz), 6.76 (1H, d, J=2.46 Hz), 6.86 (1H, d, J=9.31 Hz), 7.66-7.69 (2H, m), 8.08 (1H, d, J=8.76 Hz), 8.11 (1H, s); ESIMS found for C₂₂H₂₃F₅N₈O₂ m/z 527.2 (M+1).

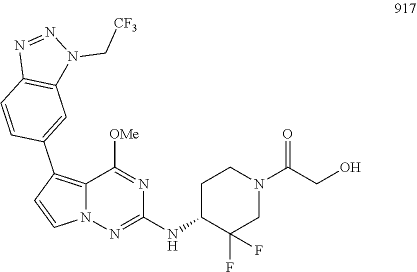

(R)-1-(3,3-Difluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-hydroxyethan-1-one 917.

Fluffy white solid (8 mg, 0.015 mmol, 42.8% yield). ¹H NMR (499 MHz, DMSO-d₆) δ ppm 1.60-1.85 (1H, m), 1.93 (1H, br t, J=13.42 Hz), 3.06 (1H, br t, J=11.09 Hz), 3.59-3.85 (1H, m), 3.97 (3H, s), 4.05-4.31 (3H, m), 4.44-4.66 (2H, m), 4.82 (1H, br d, J=4.93 Hz), 5.88 (2H, q, J=9.22 Hz), 6.77 (1H, d, J=2.46 Hz), 7.05 (1H, d, J=9.58 Hz), 7.66 (1H, d, J=2.46 Hz), 7.68 (1H, dd, J=8.76, 1.37 Hz), 8.08 (1H, d, J=8.76 Hz), 8.11 (1H, s); ESIMS found for C₂₂H₂₁F₅N₈O₃ m/z 541.2 (M+1).

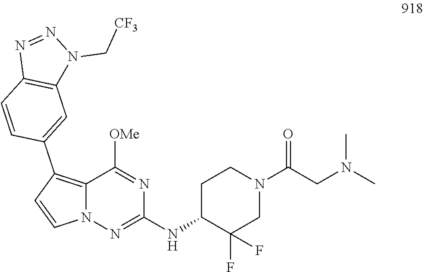

(R)-1-(3,3-Difluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-(dimethylamino)ethan-1-one 918.

White solid (7 mg, 0.012 mmol, 12.4% yield). ¹H NMR (499 MHz, DMSO-d₆) δ ppm 1.60-1.87 (1H, m), 1.89-2.00 (1H, m), 2.18 (6H, s), 2.93-3.08 (2H, m), 3.17-3.25 (1H, m), 3.62-3.77 (1H, m), 3.97 (3H, s), 4.05-4.28 (1H, m), 4.38-4.63 (2H, m), 5.87 (2H, q, J=9.03 Hz), 6.77 (1H, d, J=2.74 Hz), 6.97-7.08 (1H, m), 7.66 (1H, d, J=2.46 Hz), 7.68 (1H, dd, J=8.76, 1.37 Hz), 8.08 (1H, d, J=8.49 Hz), 8.11 (1H, s); ESIMS found for C₂₄H₂₆F₅N₉O₂ m/z 568.2 (M+1).

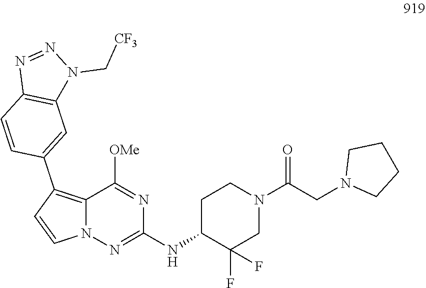

(R)-1-(3,3-Difluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-(pyrrolidin-1-yl)ethan-1-one 919.

White solid (6 mg, 0.010 mmol, 25.5% yield). ¹H NMR (499 MHz, DMSO-d₆) δ ppm 1.70 (5H, br s), 1.77-1.88 (1H, m), 1.92 (1H, br s), 2.93-3.05 (1H, m), 3.11-3.26 (2H, m), 3.38-3.51 (2H, m), 3.61-3.78 (1H, m), 3.97 (3H, s), 4.02-4.15 (1H, m), 4.25 (1H, br d, J=13.42 Hz), 4.39-4.64 (2H, m), 5.88 (2H, q, J=8.75 Hz), 6.77 (1H, br s), 6.95-7.11 (1H, m), 7.63-7.71 (2H, m), 8.08 (1H, br d, J=8.76 Hz), 8.11 (1H, s); ESIMS found for C₂₆H₂₈F₅N₉O₂ m/z 594.25 (M+1).

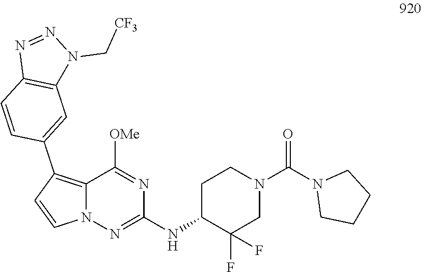

(R)-(3,3-Difluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)(pyrrolidin-1-yl) methanone 920.

White solid (14 mg, 0.024 mmol, 35.8% yield). ¹H NMR (499 MHz, DMSO-d₆) δ ppm 1.77 (5H, dt, J=10.47, 6.54 Hz), 1.81-1.96 (2H, m), 2.95-3.08 (1H, m), 3.19-3.26 (3H, m), 3.34-3.39 (1H, m), 3.63-3.73 (1H, m), 3.79-3.92 (1H, m), 3.97 (3H, s), 4.40-4.57 (1H, m), 5.88 (2H, q, J=8.80 Hz), 6.77 (1H, d, J=2.74 Hz), 6.99 (1H, d, J=9.31 Hz), 7.66-7.70 (2H, m), 8.08 (1H, d, J=8.76 Hz), 8.11 (1H, s); ESIMS found for C₂₅H₂₆F₅N₉O₂ m/z 580.2 (M+1).

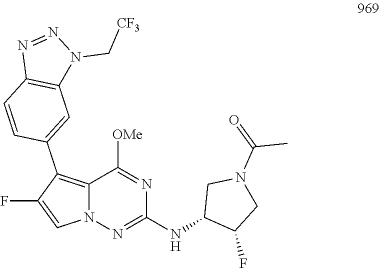 [SMILES: COc1nc(N[C@@H]2CN(C(C)=O)C[C@@H]2F)nn2cc(F)c(-c3ccc4nnn(CC(F)(F)F)c4c3)c12]

1-((3S,4R)-3-Fluoro-4-((6-fluoro-4-methoxy-5-(1-(2,2,2-trifluoroethyl)-1H-benzo[d][1,2,3]triazol-6-yl)pyrrolo[2,1-f][1,2,4]triazin-2-yl)amino)pyrrolidin-1-yl)ethan-1-one 969.

White solid (7 mg, 0.014 mmol, 45.9% yield). ¹H NMR (499 MHz, DMSO-d₆) δ ppm 1.93-2.01 (3H, m), 3.47-3.60 (1H, m), 3.61-3.75 (1H, m), 3.78-3.97 (2H, m), 3.91-3.93 (3H, m), 4.27-4.57 (1H, m), 5.22-5.49 (1H, m), 5.90 (2H, q, J=9.22 Hz), 7.22 (1H, d, J=7.39 Hz), 7.61 (1H, d, J=8.76 Hz), 7.88 (1H, dd, J=4.38, 2.74 Hz), 8.08 (1H, s), 8.13 (1H, d, J=8.49 Hz); ESIMS found for C₂₁H₁₉F₅N₈O₂ m/z 511.1 (M+1).

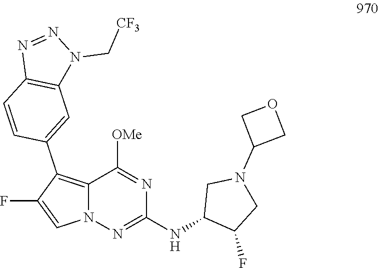 [SMILES: COc1nc(N[C@@H]2CN(C3COC3)C[C@@H]2F)nn2cc(F)c(-c3ccc4nnn(CC(F)(F)F)c4c3)c12]

6-Fluoro-N-((3R,4S)-4-fluoro-1-(oxetan-3-yl)pyrrolidin-3-yl)-4-methoxy-5-(1-(2,2,2-trifluoroethyl)-1H-benzo[d][1,2,3]triazol-6-yl)pyrrolo[2,1-f][1,2,4]triazin-2-amine 970.

White solid (7 mg, 0.013 mmol, 44.7% yield). ¹H NMR (499 MHz, DMSO-d₆) δ ppm 2.68 (1H, t, J=9.03 Hz), 2.77 (1H, ddd, J=29.85, 11.91, 1.23 Hz), 3.01 (1H, t, J=8.35 Hz), 3.17 (1H, ddd, J=32.60, 11.91, 4.52 Hz), 3.79 (1H, quin, J=6.16 Hz), 3.91 (3H, s), 4.21-4.37 (1H, m), 4.47 (2H, t, J=6.02 Hz), 4.60 (2H, t, J=6.57 Hz), 5.24 (1H, dtd, J=55.65, 4.65, 4.65, 1.60 Hz), 5.89 (2H, q, J=9.22 Hz), 6.98 (1H, d, J=7.67 Hz), 7.60 (1H, d, J=8.76 Hz), 7.87 (1H, d, J=3.01 Hz), 8.08 (1H, s), 8.13 (1H, d, J=8.76 Hz); ESIMS found for C₂₂H₂₁F₅N₈O₂ m/z 525.2 (M+1).

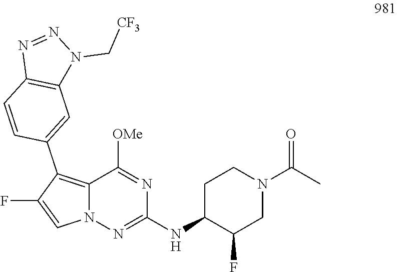 [SMILES: COc1nc(N[C@H]2CCN(C(C)=O)C[C@H]2F)nn2cc(F)c(-c3ccc4nnn(CC(F)(F)F)c4c3)c12]

1-((3R,4S)-3-Fluoro-4-((6-fluoro-4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one 981.

White solid (13 mg, 0.025 mmol, 79.7% yield). ¹H NMR (499 MHz, DMSO-d₆) δ ppm 1.65-1.73 (1H, m), 1.74-1.93 (1H, m), 1.98-2.07 (3H, m), 2.67-2.99 (1H, m), 3.22 (1H, td, J=13.14, 2.74 Hz), 3.90 (3H, s), 3.94-4.07 (1H, m), 4.07-4.19 (1H, m), 4.39-4.76 (1H, m), 4.97 (1H, d, J=48.55 Hz), 5.89 (2H, q, J=9.03 Hz), 6.93 (1H, dd, J=7.67, 4.11 Hz), 7.60 (1H, d, J=8.76 Hz), 7.84 (1H, d, J=3.01 Hz), 8.07 (1H, s), 8.13 (1H, d, J=8.76 Hz); ESIMS found for C₂₂H₂₁F₅N₈O₂ m/z 525.2 (M+1).

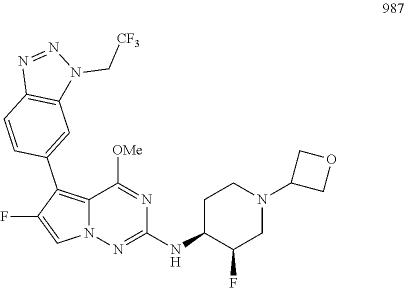 [SMILES: COc1nc(N[C@H]2CCN(C3COC3)C[C@H]2F)nn2cc(F)c(-c3ccc4nnn(CC(F)(F)F)c4c3)c12]

6-Fluoro-N-((3R,4S)-3-fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine 987.

Off-white solid (11 mg, 0.020 mmol, 65.7% yield). ¹H NMR (499 MHz, DMSO-d₆) δ ppm 1.68-1.77 (1H, m), 1.92 (1H, qd, J=12.05, 3.01 Hz), 1.99-2.05 (1H, m), 2.15 (1H, dd, J=37.00, 12.59 Hz), 2.69-2.82 (1H, m), 2.98 (1H, br t, J=9.58 Hz), 3.49 (1H, quin, J=6.02 Hz), 3.72-3.86 (1H, m), 3.90 (3H, s), 4.40 (1H, t, J=6.16 Hz), 4.46 (1H, t, J=6.02 Hz), 4.54 (2H, td, J=6.43, 2.74 Hz), 4.92 (1H, d, J=49.90 Hz), 5.89 (2H, q, J=9.22 Hz), 6.84 (1H, d, J=7.67 Hz), 7.60 (1H, d, J=8.76 Hz), 7.84 (1H, d, J=2.74 Hz), 8.07 (1H, s), 8.13 (1H, d, J=8.76 Hz); ESIMS found for C₂₃H₂₃F₅N₈O₂ m/z 539.2 (M+1).

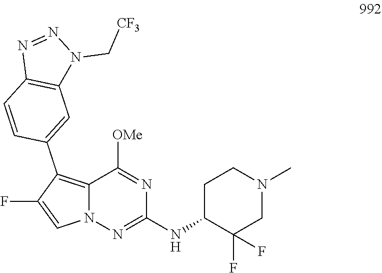 [SMILES: COc1nc(N[C@@H]2CCN(C)CC2(F)F)nn2cc(F)c(-c3ccc4nnn(CC(F)(F)F)c4c3)c12]

(R)—N-(3,3-Difluoro-1-methylpiperidin-4-yl)-6-fluoro-4-methoxy-5-(1-(2,2,2-trifluoroethyl)-1H-benzo[d][1,2,3]triazol-6-yl)pyrrolo[2,1-f][1,2,4]triazin-2-amine 992.

White solid (6 mg, 0.012 mmol, 26.5% yield). ¹H NMR (499 MHz, DMSO-d₆) δ ppm 1.77-1.90 (2H, m), 2.17 (1H, td, J=11.02, 3.15 Hz), 2.25 (3H, s), 2.37 (1H, ddd, J=26.60, 12.45, 1.37 Hz), 2.78 (1H, br d, J=10.95 Hz), 3.01-3.09 (1H, m), 3.91 (3H, s), 4.15-4.30 (1H, m), 5.89 (2H, q, J=9.22 Hz), 6.98 (1H, d, J=9.31 Hz), 7.60 (1H, d, J=8.76 Hz), 7.86 (1H, d, J=2.74 Hz), 8.07 (1H, s), 8.13 (1H, d, J=8.76 Hz); ESIMS found for C₂₁H₂₀F₆N₈O m/z 515.2 (M+1).

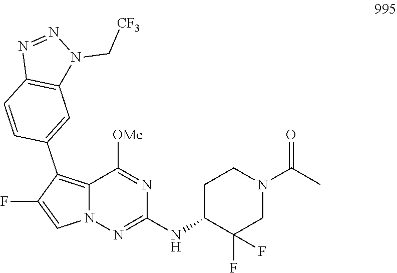 [SMILES: COc1nc(N[C@@H]2CCN(C(C)=O)CC2(F)F)nn2cc(F)c(-c3ccc4nnn(CC(F)(F)F)c4c3)c12]

(R)-1-(3,3-Difluoro-4-((6-fluoro-4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one 995.

White solid (8 mg, 0.015 mmol, 49.2% yield). ¹H NMR (499 MHz, DMSO-d₆) δ ppm 1.56-1.83 (1H, m), 1.84-2.00 (1H, m), 2.02-2.09 (3H, m), 2.91-3.05 (1H, m), 3.63-3.89 (1H, m), 3.92 (3H, s), 4.10-4.32 (1H, m), 4.42-4.60 (2H, m), 5.89 (2H, q, J=9.22 Hz), 7.14 (1H, dd, J=9.31, 2.46 Hz), 7.61 (1H, d, J=8.76 Hz), 7.86 (1H, d, J=2.19 Hz), 8.08 (1H, s), 8.13 (1H, d, J=8.49 Hz); ESIMS found for C₂₂H₂₀F₆N₈O₂ m/z 543.2 (M+1).

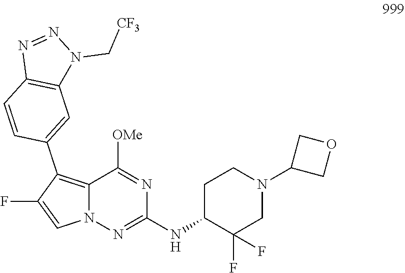 [SMILES: COc1nc(N[C@@H]2CCN(C3COC3)CC2(F)F)nn2cc(F)c(-c3ccc4nnn(CC(F)(F)F)c4c3)c12]

(R)—N-(3,3-Difluoro-1-(oxetan-3-yl)piperidin-4-yl)-6-fluoro-4-methoxy-5-(1-(2,2,2-trifluoroethyl)-1H-benzo[d][1,2,3]triazol-6-yl)pyrrolo[2,1-f][1,2,4]triazin-2-amine 999.

White solid (7 mg, 0.013 mmol, 42.0% yield). ¹H NMR (499 MHz, DMSO-d₆) δ ppm 1.76-1.85 (1H, m), 1.86-1.92 (1H, m), 2.10-2.21 (1H, m), 2.39 (1H, dd, J=27.15, 11.50 Hz), 2.70-2.82 (1H, m), 2.95-3.08 (1H, m), 3.60 (1H, quin, J=6.23 Hz), 3.92 (3H, s), 4.20-4.36 (1H, m), 4.44 (2H, dt, J=15.33, 6.16 Hz), 4.55 (2H, td, J=6.57, 3.56 Hz), 5.89 (2H, q, J=9.13 Hz), 7.04 (1H, d, J=9.58 Hz), 7.60 (1H, d, J=8.76 Hz), 7.86 (1H, d, J=2.74 Hz), 8.08 (1H, s), 8.13 (1H, d, J=8.76 Hz); ESIMS found for C₂₃H₂₂F₆N₈O₂ m/z 557.1 (M+1).

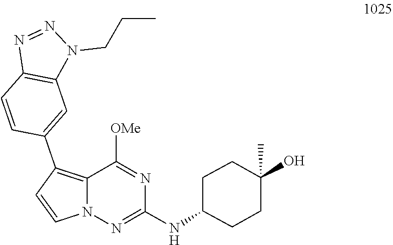 [SMILES: CCCn1nnc2ccc(-c3ccn4nc(N[C@H]5CC[C@](C)(O)CC5)nc(OC)c34)cc21]

(1r,4r)-4-((4-Methoxy-5-(1-propyl-1H-benzo[d][1,2,3]triazol-6-yl)pyrrolo[2,1-f][1,2,4]triazin-2-yl)amino)-1-methylcyclohexan-1-ol 1025.

Fluffy white solid (13 mg, 0.030 mmol, 61.8% yield). ¹H NMR (499 MHz, DMSO-d₆) δ ppm 0.89 (3H, t, J=7.39 Hz), 1.15 (3H, s), 1.37-1.52 (4H, m), 1.56-1.66 (2H, m), 1.82-1.91 (2H, m), 1.96 (2H, sxt, J=7.23 Hz), 3.61-3.71 (1H, m), 3.94 (3H, s), 4.23 (1H, s), 4.68 (2H, t, J=6.98 Hz), 6.48 (1H, d, J=7.94 Hz), 6.73 (1H, d, J=2.74 Hz), 7.56-7.61 (1H, m), 7.64 (1H, d, J=2.46 Hz), 7.96-8.00 (2H, m); ESIMS found for C₂₃H₂₉N₇O₂ m/z 436.2 (M+1).

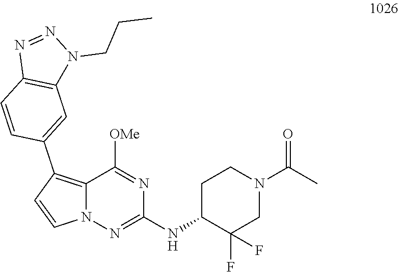 [SMILES: CCCn1nnc2ccc(-c3ccn4nc(N[C@@H]5CCN(C(C)=O)CC5(F)F)nc(OC)c34)cc21]

(R)-1-(3,3-Difluoro-4-((4-methoxy-5-(1-propyl-1H-benzo[d][1,2,3]triazol-6-yl)pyrrolo[2,1-f][1,2,4]triazin-2-yl)amino)piperidin-1-yl)ethan-1-one 1026.

Fluffy white solid (8 mg, 0.017 mmol, 39.6% yield). ¹H NMR (499 MHz, DMSO-d₆) δ ppm 0.89 (3H, t, J=7.39 Hz), 1.58-1.83 (1H, m), 1.84-1.92 (1H, m), 1.97 (2H, q, J=7.25 Hz), 2.04-2.10 (3H, m), 2.94-3.05 (1H, m), 3.65-3.90 (1H, m), 3.98 (3H, s), 4.10-4.32 (1H, m), 4.44-4.60 (2H, m), 4.68 (2H, t, J=6.98 Hz), 6.79 (1H, d, J=2.74 Hz), 7.00 (1H, d, J=9.31 Hz), 7.59-7.62 (1H, m), 7.65 (1H, dd, J=2.46, 1.37 Hz), 7.98-8.01 (2H, m); ESIMS found for C₂₃H₂₆F₂N₈O₂ m/z 485.2+(M+1).

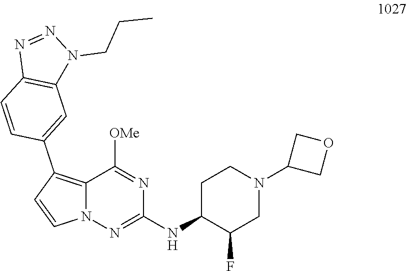 [SMILES: CCCn1nnc2ccc(-c3ccn4nc(N[C@H]5CCN(C6COC6)C[C@H]5F)nc(OC)c34)cc21]

N-((3R,4S)-3-Fluoro-1-(oxetan-3-yl)piperidin-4-yl)-4-methoxy-5-(1-propyl-1H-benzo[d][1,2,3]triazol-6-yl)pyrrolo[2,1-f][1,2,4]triazin-2-amine 1027.

Fluffy white solid (10 mg, 0.021 mmol, 49.4% yield). ¹H NMR (499 MHz, DMSO-d₆) δ ppm 0.89 (3H, t, J=7.39 Hz), 1.67-1.78 (1H, m), 1.87-1.94 (1H, m), 1.94-1.99 (2H, m), 2.00-2.06 (1H, m), 2.16 (1H, dd, J=37.00, 12.32 Hz), 2.76 (1H, br d, J=12.32 Hz), 2.94-3.05 (1H, m), 3.50 (1H, quin, J=6.30 Hz), 3.74-3.90 (1H, m), 3.96 (3H, s), 4.40 (1H, t, J=6.16 Hz), 4.46 (1H, t, J=6.02 Hz), 4.54 (2H, td, J=6.57, 3.01 Hz), 4.68 (2H, t, J=6.98 Hz), 4.94 (1H, d, J=49.35 Hz), 6.69 (1H, d, J=7.67 Hz), 6.76 (1H, d, J=2.74 Hz), 7.58-7.61 (1H, m), 7.64 (1H, d, J=2.74 Hz), 7.97-8.00 (2H, m); ESIMS found for C₂₄H₂₉FN₈O₂ m/z 481.3 (M+1).

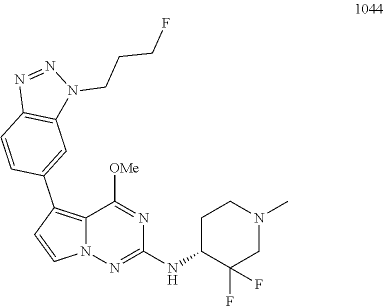

(R)—N-(3,3-Difluoro-1-methylpiperidin-4-yl)-5-(1-(3-fluoropropyl)-1H-benzo[d][1,2,3]triazol-6-yl)-4-methoxypyrrolo[2,1-f][1,2,4]triazin-2-amine 1044.

White solid (20 mg, 0.042 mmol, 40.0% yield). ¹H NMR (499 MHz, DMSO-d₆) δ ppm 1.77-1.91 (2H, m), 2.11-2.22 (1H, m), 2.26 (3H, s), 2.28-2.44 (3H, m), 2.79 (1H, br d, J=11.77 Hz), 3.01-3.10 (1H, m), 3.97 (3H, s), 4.17-4.33 (1H, m), 4.51 (2H, dt, J=47.45, 5.61 Hz), 4.83 (2H, t, J=6.98 Hz), 6.78 (1H, d, J=2.46 Hz), 6.84 (1H, d, J=9.31 Hz), 7.60-7.64 (1H, m), 7.66 (1H, d, J=2.46 Hz), 7.98-8.02 (2H, m); ESIMS found for C₂₂H₂₅F₃N₈O m/z 475.2 (M+1).

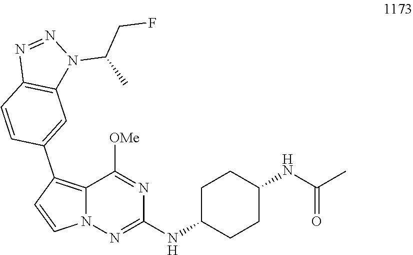 [SMILES: COc1nc(N[C@H]2CC[C@@H](NC(C)=O)CC2)nn2ccc(-c3ccc4nnn([C@@H](C)CF)c4c3)c12]

N-(cis-4-((5-(1-((S)-1-fluoropropan-2-yl)-1H-benzo[d][1,2,3]triazol-6-yl)-4-methoxypyrrolo[2,1-f][1,2,4]triazin-2-yl)amino)cyclohexyl)acetamide 1173.

White solid (8 mg, 0.017 mmol, 14.8% yield). ¹H NMR (499 MHz, DMSO-d₆) δ ppm 1.51-1.59 (2H, m), 1.62-1.70 (2H, m), 1.68 (3H, dd, J=6.98, 1.23 Hz), 1.74-1.80 (4H, m), 1.81 (3H, s), 3.68 (2H, dt, J=6.64, 3.39 Hz), 3.95 (3H, s), 4.83-4.92 (1H, m), 4.92-5.01 (1H, m), 5.42-5.54 (1H, m), 6.45 (1H, d, J=6.30 Hz), 6.74 (1H, d, J=2.74 Hz), 7.61 (1H, dd, J=8.76, 1.37 Hz), 7.64 (1H, d, J=2.46 Hz), 7.70 (1H, d, J=7.12 Hz), 8.01 (1H, d, J=8.76 Hz), 8.04 (1H, s); ESIMS found for C₂₄H₂₉FN₈O₂ m/z 481.3 (M+1).

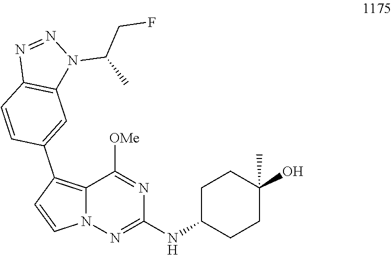 [SMILES: COc1nc(N[C@H]2CC[C@](C)(O)CC2)nn2ccc(-c3ccc4nnn([C@@H](C)CF)c4c3)c12]

(1S,4r)-4-((5-(1-((S)-1-Fluoropropan-2-yl)-1H-benzo[d][1,2,3]triazol-6-yl)-4-methoxypyrrolo[2,1-f][1,2,4]triazin-2-yl)amino)-1-methylcyclohexan-1-ol 1175.

Fluffy white solid (13 mg, 0.029 mmol, 59.4% yield). ¹H NMR (499 MHz, DMSO-d₆) δ ppm 1.15 (3H, s), 1.38-1.52 (4H, m), 1.55-1.64 (2H, m), 1.68 (3H, dd, J=6.98, 1.23 Hz), 1.81-1.93 (2H, m), 3.59-3.70 (1H, m), 3.93 (3H, s), 4.23 (1H, s), 4.82-5.02 (2H, m), 5.41-5.54 (1H, m), 6.48 (1H, d, J=7.94 Hz), 6.73 (1H, d, J=2.74 Hz), 7.59-7.62 (1H, m), 7.64 (1H, d, J=2.74 Hz), 7.99-8.02 (1H, m), 8.03 (1H, s); ESIMS found for C₂₃H₂₈FN₇O₂ m/z 454.2 (M+1).

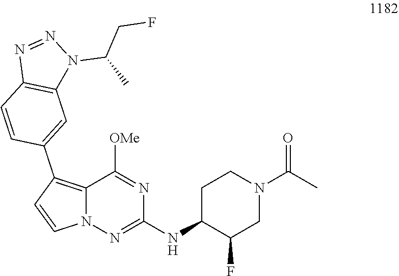 [SMILES: COc1nc(N[C@H]2CCN(C(C)=O)C[C@H]2F)nn2ccc(-c3ccc4nnn([C@@H](C)CF)c4c3)c12]

1-((3R,4S)-3-Fluoro-4-((5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one 1182.

White solid (8 mg, 0.017 mmol, 31.4% yield). ¹H NMR (499 MHz, DMSO-d₆) δ ppm 1.68 (3H, dd, J=6.98, 1.23 Hz), 1.70-1.75 (1H, m), 1.76-1.90 (1H, m), 1.99-2.07 (3H, m), 2.66-2.99 (1H, m), 3.15-3.26 (1H, m), 3.87-4.03 (1H, m), 3.95 (3H, s), 4.05-4.17 (1H, m), 4.40-4.75 (1H, m), 4.83-4.91 (1H, m), 4.92-5.06 (2H, m), 5.41-5.55 (1H, m), 6.77 (1H, d, J=2.74 Hz), 6.80 (1H, dd, J=7.80, 2.60 Hz), 7.61 (1H, dd, J=8.76, 1.37 Hz), 7.64 (1H, d, J=2.74 Hz), 8.02 (1H, d, J=8.76 Hz), 8.04 (1H, s); ESIMS found for C₂₃H₂₆F₂N₈O₂ m/z 485.25 (M+1).

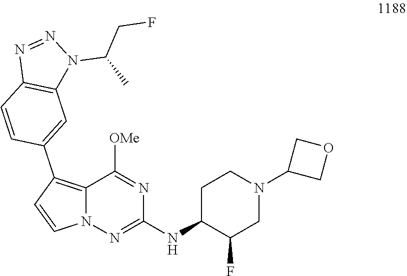 [SMILES: COc1nc(N[C@H]2CCN(C3COC3)C[C@H]2F)nn2ccc(-c3ccc4nnn([C@@H](C)CF)c4c3)c12]

N-((3R,4S)-3-Fluoro-1-(oxetan-3-yl)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine 1188.

Fluffy white solid (10 mg, 0.020 mmol, 47.6% yield). ¹H NMR (499 MHz, DMSO-d₆) δ ppm 1.68 (3H, dd, J=6.98, 1.23 Hz), 1.70-1.76 (1H, m), 1.92 (1H, qd, J=12.09, 3.42 Hz), 1.99-2.06 (1H, m), 2.16 (1H, dd, J=37.00, 12.87 Hz), 2.76 (1H, br d, J=10.68 Hz), 2.99 (1H, br t, J=10.27 Hz), 3.49 (1H, quin, J=6.37 Hz), 3.74-3.90 (1H, m), 3.95 (3H, s), 4.40 (1H, t, J=6.16 Hz), 4.46 (1H, t, J=6.16 Hz), 4.54 (2H, td, J=6.43, 3.01 Hz), 4.83-5.01 (3H, m), 5.47 (1H, dtd, J=16.19, 7.31, 7.31, 4.24 Hz), 6.69 (1H, d, J=7.94 Hz), 6.76 (1H, d, J=2.46 Hz), 7.61 (1H, dd, J=8.76, 1.37 Hz), 7.64 (1H, d, J=2.74 Hz), 8.01 (1H, d, J=8.49 Hz), 8.04 (1H, s); ESIMS found for C₂₄H₂₈F₂N₈O₂ m/z 499.2 (M+1).

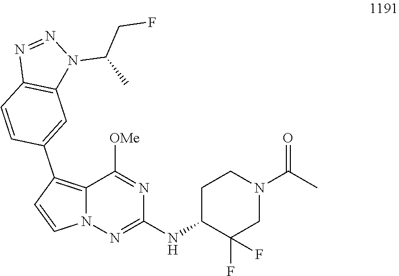 [SMILES: COc1nc(N[C@@H]2CCN(C(C)=O)CC2(F)F)nn2ccc(-c3ccc4nnn([C@@H](C)CF)c4c3)c12]

1-((R)-3,3-Difluoro-4-((5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one 1191.

Fluffy white solid (9 mg, 0.018 mmol, 43.0% yield). ¹H NMR (499 MHz, DMSO-d₆) δ ppm 1.68 (3H, dd, J=7.12, 1.10 Hz), 1.73-1.84 (1H, m), 1.85-1.99 (1H, m), 2.02-2.09 (3H, m), 2.93-3.06 (1H, m), 3.64-3.92 (1H, m), 3.97 (3H, s), 4.07-4.33 (1H, m), 4.44-4.62 (2H, m), 4.83-5.03 (2H, m), 5.42-5.55 (1H, m), 6.79 (1H, d, J=2.74 Hz), 7.00 (1H, d, J=9.31 Hz), 7.62 (1H, dd, J=8.76, 1.37 Hz), 7.65 (1H, dd, J=2.60, 1.23 Hz), 8.02 (1H, d, J=8.49 Hz), 8.05 (1H, s); ESIMS found for C₂₃H₂₅F₃N₈O₂ m/z 503.2 (M+1).

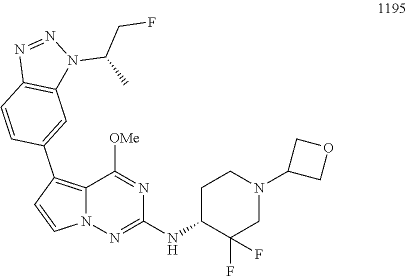 [SMILES: COc1nc(N[C@@H]2CCN(C3COC3)CC2(F)F)nn2ccc(-c3ccc4nnn([C@@H](C)CF)c4c3)c12]

N—((R)-3,3-Difluoro-1-(oxetan-3-yl)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine 1195.

White solid (18 mg, 0.035 mmol, 32.6% yield). ¹H NMR (499 MHz, DMSO-d₆) δ ppm 1.68 (3H, dd, J=7.12, 1.10 Hz), 1.76-1.86 (1H, m), 1.86-1.93 (1H, m), 2.16 (1H, br t, J=10.54 Hz), 2.41 (1H, dd, J=26.85, 11.77 Hz), 2.76 (1H, br d, J=12.05 Hz), 2.94-3.08 (1H, m), 3.60 (1H, quin, J=6.30 Hz), 3.97 (3H, s), 4.23-4.37 (1H, m), 4.44 (2H, dt, J=15.06, 6.16 Hz), 4.55 (2H, td, J=6.57, 3.56 Hz), 4.83-5.02 (2H, m), 5.41-5.53 (1H, m), 6.78 (1H, d, J=2.46 Hz), 6.90 (1H, d, J=9.31 Hz), 7.61 (1H, dd, J=8.76, 1.37 Hz), 7.65 (1H, d, J=2.74 Hz), 8.02 (1H, d, J=8.76 Hz), 8.05 (1H, s); ESIMS found for C₂₄H₂₇F₃N₈O₂ m/z 517.25 (M+1).

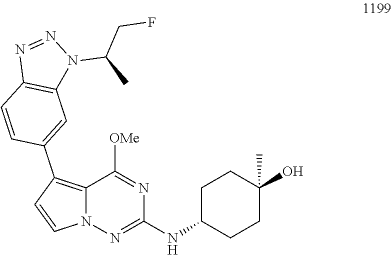 [SMILES: COc1nc(N[C@H]2CC[C@](C)(O)CC2)nn2ccc(-c3ccc4nnn([C@H](C)CF)c4c3)c12]

(1R,4r)-4-((5-(1-((R)-1-Fluoropropan-2-yl)-1H-benzo[d][1,2,3]triazol-6-yl)-4-methoxypyrrolo[2,1-f][1,2,4]triazin-2-yl)amino)-1-methylcyclohexan-1-ol 1199.

Fluffy white solid (15 mg, 0.033 mmol, 68.5% yield). ¹H NMR (499 MHz, DMSO-d₆) δ ppm 1.15 (3H, s), 1.37-1.53 (4H, m), 1.56-1.65 (2H, m), 1.68 (3H, dd, J=7.12, 1.10 Hz), 1.82-1.93 (2H, m), 3.60-3.71 (1H, m), 3.93 (3H, s), 4.23 (1H, s), 4.81-5.03 (2H, m), 5.41-5.54 (1H, m), 6.48 (1H, d, J=7.94 Hz), 6.73 (1H, d, J=2.74 Hz), 7.60 (1H, dd, J=8.62, 1.51 Hz), 7.64 (1H, d, J=2.46 Hz), 8.00 (1H, d, J=8.76 Hz), 8.03 (1H, s); ESIMS found for C₂₃H₂₈FN₇O₂ m/z 454.2 (M+1).

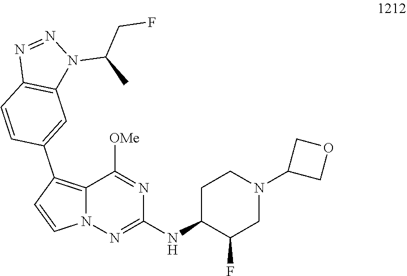 [SMILES: COc1nc(N[C@H]2CCN(C3COC3)C[C@H]2F)nn2ccc(-c3ccc4nnn([C@H](C)CF)c4c3)c12]

N-((3R,4S)-3-Fluoro-1-(oxetan-3-yl)piperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine 1212.

Fluffy white solid (9 mg, 0.018 mmol, 42.8% yield). ¹H NMR (499 MHz, DMSO-d₆) δ ppm 1.68 (3H, dd, J=7.12, 1.10 Hz), 1.70-1.76 (1H, m), 1.92 (1H, qd, J=12.18, 3.42 Hz), 1.99-2.06 (1H, m), 2.16 (1H, dd, J=37.00, 12.59 Hz), 2.76 (1H, br d, J=10.13 Hz), 2.93-3.05 (1H, m), 3.49 (1H, quin, J=6.37 Hz), 3.72-3.92 (1H, m), 3.95 (3H, s), 4.40 (1H, t, J=6.16 Hz), 4.46 (1H, t, J=6.16 Hz), 4.54 (2H, td, J=6.43, 3.01 Hz), 4.83-5.01 (3H, m), 5.41-5.54 (1H, m), 6.69 (1H, d, J=7.94 Hz), 6.76 (1H, d, J=2.74 Hz), 7.61 (1H, dd, J=8.76, 1.37 Hz), 7.64 (1H, d, J=2.74 Hz), 8.01 (1H, d, J=8.49 Hz), 8.04 (1H, s); ESIMS found for C₂₄H₂₈F₂N₈O₂ m/z 499.2 (M+1).

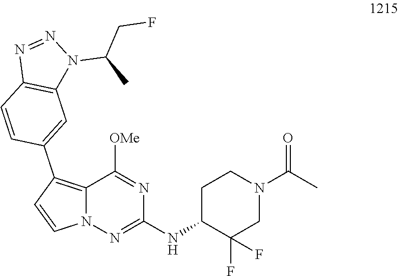 [SMILES: COc1nc(N[C@@H]2CCN(C(C)=O)CC2(F)F)nn2ccc(-c3ccc4nnn([C@H](C)CF)c4c3)c12]

1-((R)-3,3-Difluoro-4-((5-(1-((R)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one 1215.

Fluffy white solid (7 mg, 0.014 mmol, 33.4% yield). ¹H NMR (499 MHz, DMSO-d₆) δ ppm 1.68 (3H, dd, J=6.98, 1.23 Hz), 1.72-1.84 (1H, m), 1.84-2.00 (1H, m), 2.03-2.09 (3H, m), 2.94-3.04 (1H, m), 3.62-3.91 (1H, m), 3.97 (3H, s), 4.10-4.32 (1H, m), 4.45-4.62 (2H, m), 4.82-5.02 (2H, m), 5.41-5.56 (1H, m), 6.79 (1H, d, J=2.74 Hz), 7.00 (1H, d, J=9.31 Hz), 7.62 (1H, dd, J=8.76, 1.37 Hz), 7.65 (1H, dd, J=2.60, 1.23 Hz), 8.02 (1H, d, J=8.49 Hz), 8.05 (1H, s); ESIMS found for C₂₃H₂₅F₃N₈O₂ m/z 503.2 (M+1).

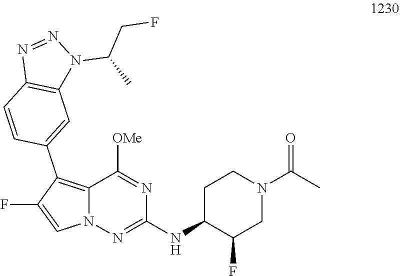 [SMILES: COc1nc(N[C@H]2CCN(C(C)=O)C[C@H]2F)nn2cc(F)c(-c3ccc4nnn([C@@H](C)CF)c4c3)c12]

1-((3R,4S)-3-Fluoro-4-((6-fluoro-5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one 1230.

Fluffy white solid (8 mg, 0.016 mmol, 73.3% yield). ¹H NMR (499 MHz, DMSO-d₆) δ ppm 1.68 (3H, dd, J=7.12, 1.10 Hz), 1.70-1.79 (1H, m), 1.80-1.91 (1H, m), 1.98-2.06 (3H, m), 2.68-2.98 (1H, m), 3.15-3.26 (1H, m), 3.91 (3H, s), 3.94-4.02 (1H, m), 4.02-4.19 (1H, m), 4.41-4.76 (1H, m), 4.83-5.00 (2H, m), 4.91-5.05 (1H, m), 5.42-5.56 (1H, m), 6.88-6.95 (1H, m), 7.54 (1H, d, J=8.76 Hz), 7.83 (1H, d, J=2.74 Hz), 8.03 (1H, s), 8.07 (1H, d, J=8.76 Hz); ESIMS found for C₂₃H₂₅F₃N₈O₂ m/z 503.2 (M+1).

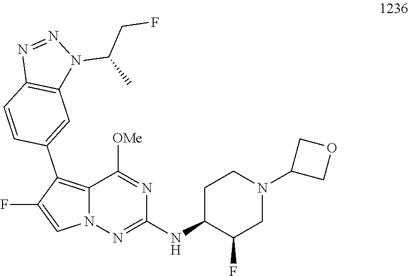 [SMILES: COc1nc(N[C@H]2CCN(C3COC3)C[C@H]2F)nn2cc(F)c(-c3ccc4nnn([C@@H](C)CF)c4c3)c12]

6-fluoro-N-((3R,4S)-3-Fluoro-1-(oxetan-3-yl)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine 1236.

Fluffy white solid (7 mg, 0.014 mmol, 41.6% yield). ¹H NMR (499 MHz, DMSO-d₆) δ ppm 1.68 (3H, dd, J=6.98, 1.23 Hz), 1.70-1.75 (1H, m), 1.87-1.97 (1H, m), 1.98-2.05 (1H, m), 2.15 (1H, dd, J=36.45, 12.87 Hz), 2.76 (1H, br d, J=12.32 Hz), 2.94-3.05 (1H, m), 3.49 (1H, quin, J=6.30 Hz), 3.71-3.87 (1H, m), 3.91 (3H, s), 4.40 (1H, t, J=6.16 Hz), 4.46 (1H, t, J=6.16 Hz), 4.54 (2H, td, J=6.57, 3.01 Hz), 4.81-5.00 (3H, m), 5.43-5.56 (1H, m), 6.81 (1H, d, J=7.94 Hz), 7.54 (1H, d, J=8.76 Hz), 7.83 (1H, d, J=3.01 Hz), 8.03 (1H, s), 8.06 (1H, d, J=8.76 Hz); ESIMS found for C₂₄H₂₇F₃N₈O₂ m/z 517.2 (M+1).

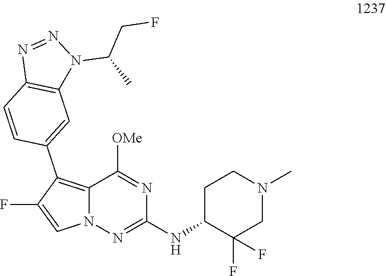 [SMILES: COc1nc(N[C@@H]2CCN(C)CC2(F)F)nn2cc(F)c(-c3ccc4nnn([C@@H](C)CF)c4c3)c12]

N—((R)-3,3-Difluoro-1-methylpiperidin-4-yl)-6-fluoro-5-(1-((S)-1-fluoropropan-2-yl)-1H-benzo[d][1,2,3]triazol-6-yl)-4-methoxypyrrolo[2,1-f][1,2,4]triazin-2-amine 1237.

Fluffy white solid (6 mg, 0.012 mmol, 58.3% yield). ¹H NMR (499 MHz, DMSO-d₆) δ ppm 1.68 (3H, dd, J=6.98, 1.23 Hz), 1.75-1.90 (2H, m), 2.10-2.21 (1H, m), 2.26 (3H, s), 2.37 (1H, dd, J=26.85, 12.32 Hz), 2.72-2.84 (1H, m), 3.00-3.11 (1H, m), 3.92 (3H, s), 4.14-4.30 (1H, m), 4.81-5.02 (2H, m), 5.40-5.57 (1H, m), 6.95 (1H, d, J=9.31 Hz), 7.54 (1H, d, J=8.76 Hz), 7.85 (1H, d, J=3.01 Hz), 8.04 (1H, s), 8.06 (1H, d, J=8.76 Hz); ESIMS found for C₂₂H₂₄F₄N₈O m/z 493.2 (M+1).

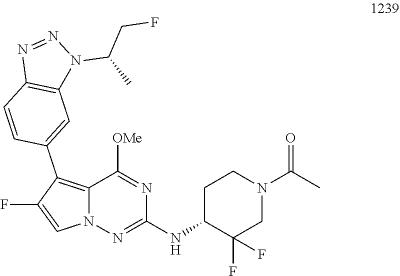 [SMILES: COc1nc(N[C@@H]2CCN(C(C)=O)CC2(F)F)nn2cc(F)c(-c3ccc4nnn([C@@H](C)CF)c4c3)c12]

1-((R)-3,3-Difluoro-4-((6-fluoro-5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one 1239.

Fluffy white solid (9 mg, 0.017 mmol, 82.7% yield). ¹H NMR (499 MHz, DMSO-d₆) δ ppm 1.68 (3H, dd, J=7.12, 1.37 Hz), 1.73-1.84 (1H, m), 1.84-1.99 (1H, m), 2.02-2.09 (3H, m), 2.92-3.04 (1H, m), 3.62-3.90 (1H, m), 3.93 (3H, s), 4.10-4.31 (1H, m), 4.44-4.58 (2H, m), 4.82-5.02 (2H, m), 5.42-5.56 (1H, m), 7.08-7.14 (1H, m), 7.55 (1H, d, J=8.49 Hz), 7.81-7.86 (1H, m), 8.04 (1H, s), 8.07 (1H, d, J=8.76 Hz); ESIMS found for C₂₃H₂₄F₄N₈O₂ m/z 521.2 (M+1).

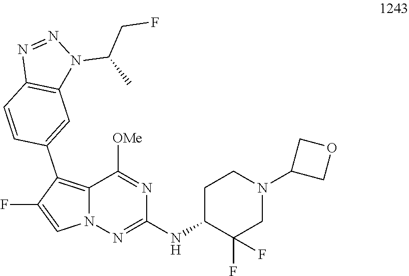 [SMILES: COc1nc(N[C@@H]2CCN(C3COC3)CC2(F)F)nn2cc(F)c(-c3ccc4nnn([C@@H](C)CF)c4c3)c12]

N—((R)-3,3-Difluoro-1-(oxetan-3-yl)piperidin-4-yl)-6-fluoro-5-(1-((S)-1-fluoropropan-2-yl)-1H-benzo[d][1,2,3]triazol-6-yl)-4-methoxypyrrolo[2,1-f][1,2,4]triazin-2-amine 1243.

Fluffy white solid (7 mg, 0.013 mmol, 36.9% yield). ¹H NMR (499 MHz, DMSO-d₆) δ ppm 1.68 (3H, dd, J=7.12, 1.37 Hz), 1.76-1.85 (1H, m), 1.85-1.92 (1H, m), 2.09-2.21 (1H, m), 2.39 (1H, dd, J=27.45, 11.77 Hz), 2.76 (1H, br d, J=11.50 Hz), 2.95-3.08 (1H, m), 3.60 (1H, quin, J=6.37 Hz), 3.93 (3H, s), 4.21-4.37 (1H, m), 4.44 (2H, dt, J=15.19, 6.09 Hz), 4.55 (2H, td, J=6.64, 3.70 Hz), 4.82-5.01 (2H, m), 5.41-5.57 (1H, m), 7.01 (1H, d, J=9.31 Hz), 7.50-7.59 (1H, m), 7.84 (1H, d, J=2.74 Hz), 8.04 (1H, s), 8.06 (1H, d, J=8.49 Hz); ESIMS found for C₂₄H₂₆F₄N₈O₂ m/z 535.2 (M+1).

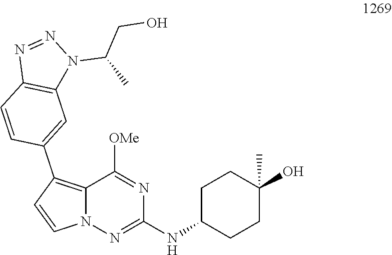 [SMILES: COc1nc(N[C@H]2CC[C@](C)(O)CC2)nn2ccc(-c3ccc4nnn([C@@H](C)CO)c4c3)c12]

(1S,4r)-4-((5-(1-((S)-1-Hydroxypropan-2-yl)-1H-benzo[d][1,2,3]triazol-6-yl)-4-methoxypyrrolo[2,1-f][1,2,4]triazin-2-yl)amino)-1-methylcyclohexan-1-ol 1269.

Fluffy white solid (10 mg, 0.022 mmol, 45.9% yield). ¹H NMR (499 MHz, DMSO-d₆) δ ppm 1.15 (3H, s), 1.37-1.52 (4H, m), 1.57-1.62 (2H, m), 1.63 (3H, d, J=6.84 Hz), 1.82-1.93 (2H, m), 3.65 (1H, dt, J=8.01, 4.07 Hz), 3.83-3.88 (1H, m), 3.89-3.93 (1H, m), 3.94 (3H, s), 4.23 (1H, s), 5.01-5.11 (2H, m), 6.47 (1H, d, J=7.94 Hz), 6.72 (1H, d, J=2.74 Hz), 7.53-7.59 (1H, m), 7.64 (1H, d, J=2.46 Hz), 7.94-8.00 (2H, m); ESIMS found for C₂₃H₂₉N₇O₃ m/z 452.2 (M+1).

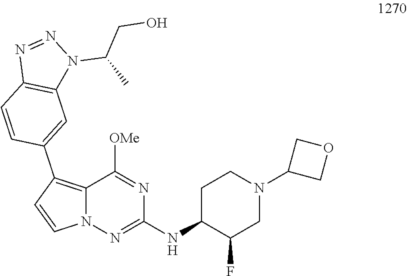 [SMILES: COc1nc(N[C@H]2CCN(C3COC3)C[C@H]2F)nn2ccc(-c3ccc4nnn([C@@H](C)CO)c4c3)c12]

(S)-2-(6-(2-(((3R,4S)-3-Fluoro-1-(oxetan-3-yl)piperidin-4-yl)amino)-4-methoxypyrrolo[2,1-f][1,2,4]triazin-5-yl)-1H-benzo[d][1,2,3]triazol-1-yl)propan-1-ol 1270.

Fluffy white solid (8 mg, 0.016 mmol, 38.2% yield). ¹H NMR (499 MHz, DMSO-d₆) δ ppm 1.63 (3H, d, J=7.12 Hz), 1.68-1.78 (1H, m), 1.92 (1H, qd, J=12.14, 3.29 Hz), 1.98-2.06 (1H, m), 2.16 (1H, dd, J=36.75, 12.87 Hz), 2.76 (1H, br d, J=11.23 Hz), 2.92-3.06 (1H, m), 3.50 (1H, quin, J=6.37 Hz), 3.76-3.87 (1H, m), 3.86 (1H, td, J=10.75, 5.34 Hz), 3.90-3.95 (1H, m), 3.96 (3H, s), 4.40 (1H, t, J=6.16 Hz), 4.46 (1H, t, J=6.16 Hz), 4.54 (2H, td, J=6.57, 3.01 Hz), 4.94 (1H, d, J=49.35 Hz), 5.04-5.11 (2H, m), 6.66-6.69 (1H, m), 6.75 (1H, d, J=2.74 Hz), 7.57 (1H, dd, J=8.62, 1.51 Hz), 7.63 (1H, d, J=2.46 Hz), 7.95-8.00 (2H, m); ESIMS found for C₂₄H₂₉FN₈O₃ m/z 497.2 (M+1).

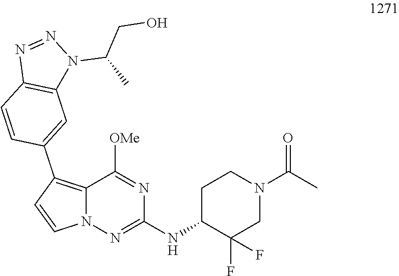 [SMILES: COc1nc(N[C@@H]2CCN(C(C)=O)CC2(F)F)nn2ccc(-c3ccc4nnn([C@@H](C)CO)c4c3)c12]

1-((R)-3,3-Difluoro-4-((5-(1-((S)-1-hydroxypropan-2-yl)-1H-benzo[d][1,2,3]triazol-6-yl)-4-methoxypyrrolo[2,1-f][1,2,4]triazin-2-yl)amino)piperidin-1-yl)ethan-1-one 1271.

Fluffy white solid (8 mg, 0.016 mmol, 38.3% yield). ¹H NMR (499 MHz, DMSO-d₆) δ ppm 1.64 (3H, d, J=6.84 Hz), 1.74-1.84 (1H, m), 1.85-1.99 (1H, m), 2.03-2.09 (3H, m), 2.94-3.04 (1H, m), 3.63-3.75 (1H, m), 3.82-3.89 (1H, m), 3.89-3.95 (1H, m), 3.98 (3H, s), 4.10-4.32 (1H, m), 4.45-4.62 (2H, m), 5.02-5.14 (2H, m), 6.77 (1H, d, J=2.46 Hz), 6.99 (1H, d, J=9.31 Hz), 7.58 (1H, dd, J=8.62, 1.51 Hz), 7.65 (1H, dd, J=2.60, 1.23 Hz), 7.98 (1H, d, J=8.76 Hz), 8.00 (1H, s); ESIMS found for C₂₃H₂₆F₂N₈O₃ m/z 501.2 (M+1).

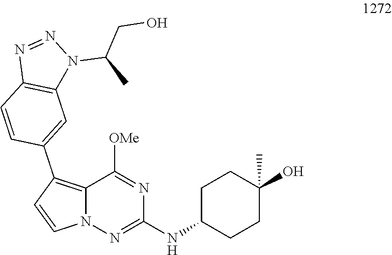 [SMILES: COc1nc(N[C@H]2CC[C@](C)(O)CC2)nn2ccc(-c3ccc4nnn([C@H](C)CO)c4c3)c12]

(1R,4r)-4-((5-(1-((R)-1-Hydroxypropan-2-yl)-1H-benzo[d][1,2,3]triazol-6-yl)-4-methoxypyrrolo[2,1-f][1,2,4]triazin-2-yl)amino)-1-methylcyclohexan-1-ol 1272.

Fluffy white solid (13 mg, 0.029 mmol, 59.7% yield). ¹H NMR (499 MHz, DMSO-d₆) δ ppm 1.15 (3H, s), 1.37-1.52 (4H, m), 1.57-1.62 (2H, m), 1.63 (3H, d, J=6.84 Hz), 1.82-1.93 (2H, m), 3.65 (1H, dt, J=7.80, 4.04 Hz), 3.83-3.88 (1H, m), 3.89-3.93 (1H, m), 3.94 (3H, s), 4.23 (1H, s), 5.01-5.12 (2H, m), 6.47 (1H, d, J=7.94 Hz), 6.72 (1H, d, J=2.74 Hz), 7.54-7.59 (1H, m), 7.64 (1H, d, J=2.46 Hz), 7.94-7.99 (2H, m); ESIMS found for C₂₃H₂₉N₇O₃ m/z 452.2 (M+1).

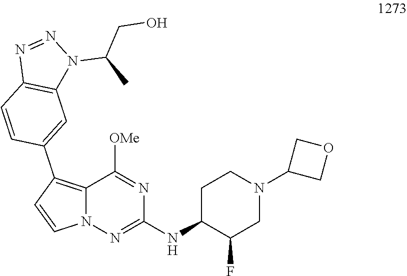 [SMILES: COc1nc(N[C@H]2CCN(C3COC3)C[C@H]2F)nn2ccc(-c3ccc4nnn([C@H](C)CO)c4c3)c12]

(R)-2-(6-(2-(((3R,4S)-3-Fluoro-1-(oxetan-3-yl)piperidin-4-yl)amino)-4-methoxypyrrolo[2,1-f][1,2,4]triazin-5-yl)-1H-benzo[d][1,2,3]triazol-1-yl)propan-1-ol1273.

Fluffy white solid (8 mg, 0.016 mmol, 38.2% yield). ¹H NMR (499 MHz, DMSO-d₆) δ ppm 1.63 (3H, d, J=6.84 Hz), 1.69-1.78 (1H, m), 1.92 (1H, qd, J=12.00, 3.42 Hz), 1.98-2.06 (1H, m), 2.16 (1H, dd, J=37.30, 12.87 Hz), 2.76 (1H, br d, J=10.40 Hz), 2.99 (1H, br t, J=10.54 Hz), 3.50 (1H, quin, J=6.30 Hz), 3.76-3.88 (1H, m), 3.83-3.89 (1H, m), 3.89-3.95 (1H, m), 3.96 (3H, s), 4.40 (1H, t, J=6.16 Hz), 4.46 (1H, t, J=6.16 Hz), 4.54 (2H, td, J=6.57, 3.01 Hz), 4.94 (1H, d, J=49.65 Hz), 5.03-5.11 (2H, m), 6.67 (1H, d, J=7.94 Hz), 6.75 (1H, d, J=2.74 Hz), 7.56-7.59 (1H, m), 7.63 (1H, d, J=2.46 Hz), 7.98 (1H, d, J=8.80 Hz), 7.99 (1H, s); ESIMS found for C₂₄H₂₉FN₈O₃ m/z 497.25 (M+1).

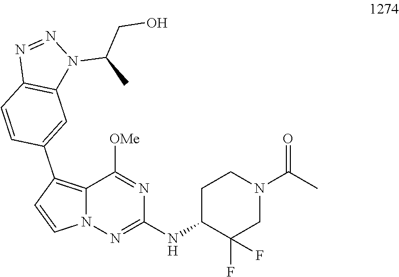 [SMILES: COc1nc(N[C@@H]2CCN(C(C)=O)CC2(F)F)nn2ccc(-c3ccc4nnn([C@H](C)CO)c4c3)c12]

1-((R)-3,3-Difluoro-4-((5-(1-((R)-1-hydroxypropan-2-yl)-1H-benzo[d][1,2,3]triazol-6-yl)-4-methoxypyrrolo[2,1-f][1,2,4]triazin-2-yl)amino)piperidin-1-yl)ethan-1-one 1274.

Fluffy white solid (10 mg, 0.020 mmol, 47.9% yield). ¹H NMR (499 MHz, DMSO-d₆) δ ppm 1.63 (3H, d, J=7.12 Hz), 1.72-1.84 (1H, m), 1.85-2.00 (1H, m), 2.03-2.09 (3H, m), 2.92-3.06 (1H, m), 3.64-3.76 (1H, m), 3.83-3.94 (2H, m), 3.99 (3H, s), 4.08-4.31 (1H, m), 4.45-4.64 (2H, m), 5.02-5.14 (2H, m), 6.77 (1H, d, J=2.46 Hz), 6.99 (1H, d, J=9.58 Hz), 7.58 (1H, dd, J=8.62, 1.51 Hz), 7.64 (1H, dd, J=2.46, 1.37 Hz), 7.98 (1H, d, J=8.49 Hz), 8.00 (1H, s); ESIMS found for C₂₃H₂₆F₂N₈O₃ m/z 501.2 (M+1

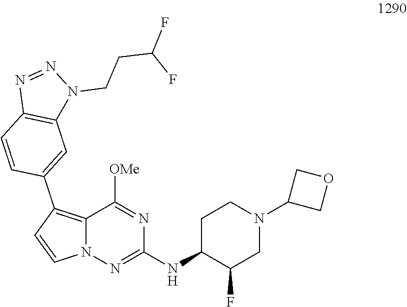 [SMILES: COc1nc(N[C@H]2CCN(C3COC3)C[C@H]2F)nn2ccc(-c3ccc4nnn(CCC(F)F)c4c3)c12]

5-(1-(3,3-Difluoropropyl)-1H-benzo[d][1,2,3]triazol-6-yl)-N-((3R,4S)-3-fluoro-1-(oxetan-3-yl)piperidin-4-yl)-4-methoxypyrrolo[2,1-f][1,2,4]triazin-2-amine 1290.

White solid (3.1 mg, 0.006 mmol, 12.0% yield). ¹H NMR (499 MHz, DMSO-d₆) δ ppm 1.73 (1H, br d, J=9.86 Hz), 1.84-1.97 (1H, m), 1.99-2.07 (1H, m), 2.16 (1H, dd, J=36.75, 12.59 Hz), 2.54-2.65 (2H, m), 2.76 (1H, br d, J=10.95 Hz), 2.99 (1H, br t, J=10.54 Hz), 3.50 (1H, quin, J=6.50 Hz), 3.74-3.91 (1H, m), 3.97 (3H, s), 4.40 (1H, t, J=6.16 Hz), 4.46 (1H, t, J=6.02 Hz), 4.54 (2H, td, J=6.43, 3.01 Hz), 4.89 (2H, t, J=6.84 Hz), 4.94 (1H, d, J=49.90 Hz), 6.24 (1H, tt, J=56.25, 4.24 Hz), 6.70 (1H, d, J=7.67 Hz), 6.77 (1H, d, J=2.46 Hz), 7.62 (1H, dd, J=9.03, 1.10 Hz), 7.64 (1H, d, J=2.46 Hz), 7.98-8.03 (2H, m); ESIMS found for C₂₄H₂₇F₃N₈O₂ m/z 517.2 (M+1).

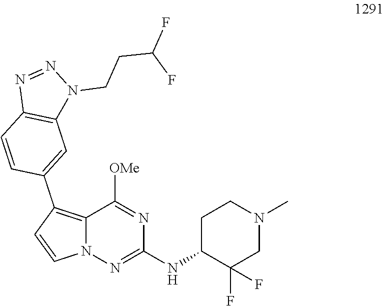

(R)—N-(3,3-Difluoro-1-methylpiperidin-4-yl)-5-(1-(3,3-difluoropropyl)-1H-benzo[d][1,2,3]triazol-6-yl)-4-methoxypyrrolo[2,1-f][1,2,4]triazin-2-amine 1291.

White solid (8.9 mg, 0.018 mmol, 33.3% yield). ¹H NMR (499 MHz, DMSO-d₆) δ ppm 1.77-1.91 (2H, m), 2.13-2.22 (1H, m), 2.26 (3H, s), 2.39 (1H, dd, J=26.30, 11.50 Hz), 2.53-2.66 (2H, m), 2.79 (1H, br d, J=11.77 Hz), 3.05 (1H, td, J=9.79, 7.53 Hz), 3.98 (3H, s), 4.17-4.34 (1H, m), 4.89 (2H, t, J=6.84 Hz), 6.24 (1H, tt, J=56.25, 4.24 Hz), 6.79 (1H, d, J=2.74 Hz), 6.84 (1H, d, J=9.31 Hz), 7.60-7.64 (1H, m), 7.66 (1H, d, J=2.46 Hz), 7.99-8.03 (2H, m); ESIMS found for C₂₂H₂₄F₄N₈O m/z 493.2 (M+1).

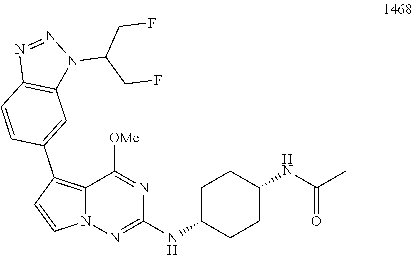 [SMILES: COc1nc(N[C@H]2CC[C@@H](NC(C)=O)CC2)nn2ccc(-c3ccc4nnn(C(CF)CF)c4c3)c12]

N-(cis-4-((5-(1-(1,3-Difluoropropan-2-yl)-1H-benzo[d][1,2,3]triazol-6-yl)-4-methoxypyrrolo[2,1-f][1,2,4]triazin-2-yl)amino)cyclohexyl)acetamide 1468.

White solid (17 mg, 0.034 mmol, 28.8% yield). ¹H NMR (499 MHz, DMSO-d₆) δ ppm 1.51-1.58 (2H, m), 1.60-1.71 (4H, m), 1.72-1.80 (2H, m), 1.81 (3H, s), 3.68 (2H, dt, J=6.84, 3.42 Hz), 3.94 (3H, s), 5.02-5.22 (4H, m), 5.72-5.84 (1H, m), 6.45 (1H, d, J=6.30 Hz), 6.74 (1H, d, J=2.46 Hz), 7.64 (1H, dd, J=8.75, 1.40 Hz), 7.64 (1H, d, J=2.74 Hz), 7.70 (1H, d, J=7.12 Hz), 8.04 (1H, d, J=8.76 Hz), 8.07 (1H, s); ESIMS found for C₂₄H₂₈F₂N₈O₂ m/z 499.3 (M+1).

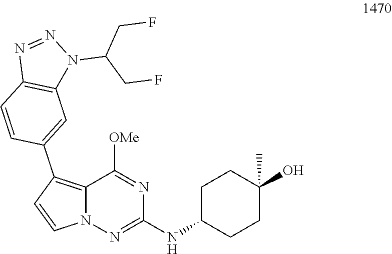 [SMILES: COc1nc(N[C@H]2CC[C@](C)(O)CC2)nn2ccc(-c3ccc4nnn(C(CF)CF)c4c3)c12]

(1r,4r)-4-((5-(1-(1,3-Difluoropropan-2-yl)-1H-benzo[d][1,2,3]triazol-6-yl)-4-methoxypyrrolo[2,1-f][1,2,4]triazin-2-yl)amino)-1-methylcyclohexan-1-ol 1470.

Fluffy white solid (15 mg, 0.032 mmol, 65.9% yield). ¹H NMR (499 MHz, DMSO-d₆) δ ppm 1.15 (3H, s), 1.36-1.54 (4H, m), 1.56-1.67 (2H, m), 1.82-1.94 (2H, m), 3.65 (1H, br dd, J=7.94, 3.83 Hz), 3.93 (3H, s), 4.23 (1H, s), 5.00-5.23 (4H, m), 5.70-5.86 (1H, m), 6.49 (1H, d, J=7.94 Hz), 6.73 (1H, d, J=2.46 Hz), 7.62-7.66 (2H, m), 8.04 (1H, d, J=8.76 Hz), 8.07 (1H, s); ESIMS found for C₂₃H₂₇F₂N₇O₂ m/z 472.2 (M+1).

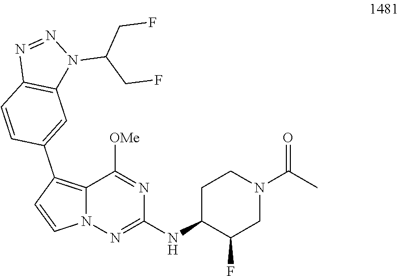 [SMILES: COc1nc(N[C@H]2CCN(C(C)=O)C[C@H]2F)nn2ccc(-c3ccc4nnn(C(CF)CF)c4c3)c12]

1-((3R,4S)-4-((5-(1-(1,3-Difluoropropan-2-yl)-1H-benzo[d][1,2,3]triazol-6-yl)-4-methoxypyrrolo[2,1-f][1,2,4]triazin-2-yl)amino)-3-fluoropiperidin-1-yl)ethan-1-one 1481.

White solid (8 mg, 0.016 mmol, 30.2% yield). ¹H NMR (499 MHz, DMSO-d₆) δ ppm 1.67-1.74 (1H, m), 1.74-1.90 (1H, m), 1.98-2.07 (3H, m), 2.67-2.99 (1H, m), 3.22 (1H, br t, J=11.64 Hz), 3.87-4.03 (1H, m), 3.95 (3H, s), 4.04-4.17 (1H, m), 4.40-4.75 (1H, m), 4.92-5.11 (3H, m), 5.11-5.22 (2H, m), 5.70-5.87 (1H, m), 6.77 (1H, d, J=2.46 Hz), 6.81 (1H, dd, J=7.67, 2.74 Hz), 7.65 (1H, dd, J=8.75, 1.40 Hz), 7.65 (1H, d, J=2.74 Hz), 8.05 (1H, d, J=8.49 Hz), 8.08 (1H, s); ESIMS found for C₂₃H₂₅F₃N₈O₂ m/z 503.2 (M+1).

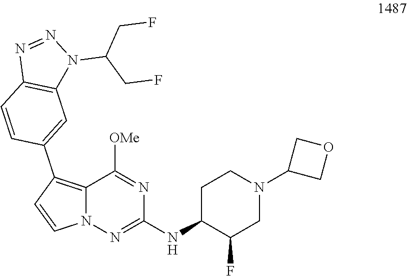 [SMILES: COc1nc(N[C@H]2CCN(C3COC3)C[C@H]2F)nn2ccc(-c3ccc4nnn(C(CF)CF)c4c3)c12]

5-(1-(1,3-Difluoropropan-2-yl)-1H-benzo[d][1,2,3]triazol-6-yl)-N-((3R,4S)-3-fluoro-1-(oxetan-3-yl)piperidin-4-yl)-4-methoxypyrrolo[2,1-f][1,2,4]triazin-2-amine 1487.

Fluffy white solid (10 mg, 0.019 mmol, 45.9% yield). ¹H NMR (499 MHz, DMSO-d₆) δ ppm 1.68-1.77 (1H, m), 1.92 (1H, qd, J=12.00, 3.42 Hz), 1.98-2.06 (1H, m), 2.16 (1H, dd, J=37.00, 12.59 Hz), 2.76 (1H, br d, J=9.58 Hz), 2.94-3.05 (1H, m), 3.49 (1H, quin, J=6.37 Hz), 3.74-3.91 (1H, m), 3.95 (3H, s), 4.40 (1H, t, J=6.16 Hz), 4.46 (1H, t, J=6.16 Hz), 4.54 (2H, td, J=6.43, 3.01 Hz), 4.94 (1H, d, J=49.60 Hz), 5.02-5.22 (4H, m), 5.72-5.86 (1H, m), 6.70 (1H, d, J=7.94 Hz), 6.76 (1H, d, J=2.74 Hz), 7.64 (1H, dd, J=8.76, 1.37 Hz), 7.64 (1H, d, J=2.74 Hz), 8.05 (1H, d, J=8.76 Hz), 8.08 (1H, s); ESIMS found for C₂₄H₂₇F₃N₈O₂ m/z 517.2 (M+1).

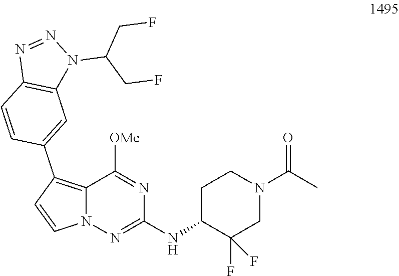 [SMILES: COc1nc(N[C@@H]2CCN(C(C)=O)CC2(F)F)nn2ccc(-c3ccc4nnn(C(CF)CF)c4c3)c12]

(R)-1-(4-((5-(1-(1,3-Difluoropropan-2-yl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one 1495.

Fluffy white solid (8 mg, 0.015 mmol, 36.9% yield). ¹H NMR (499 MHz, DMSO-d₆) δ ppm 1.57-1.83 (1H, m), 1.84-2.01 (1H, m), 2.02-2.09 (3H, m), 2.94-3.05 (1H, m), 3.63-3.90 (1H, m), 3.97 (3H, s), 4.08-4.31 (1H, m), 4.45-4.61 (2H, m), 4.99-5.24 (4H, m), 5.72-5.87 (1H, m), 6.79 (1H, d, J=2.46 Hz), 7.01 (1H, d, J=9.31 Hz), 7.65 (1H, dd, J=6.16, 1.51 Hz), 7.66 (1H, d, J=1.37 Hz), 8.05 (1H, d, J=8.76 Hz), 8.09 (1H, s); ESIMS found for C₂₃H₂₄F₄N₈O₂ m/z 521.2 (M+1).

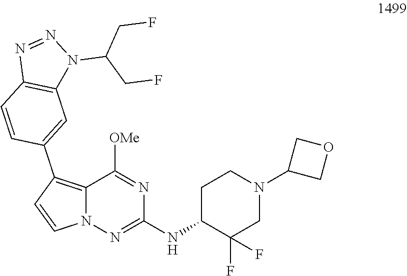 [SMILES: COc1nc(N[C@@H]2CCN(C3COC3)CC2(F)F)nn2ccc(-c3ccc4nnn(C(CF)CF)c4c3)c12]

(R)—N-(3,3-Difluoro-1-(oxetan-3-yl)piperidin-4-yl)-5-(1-(1,3-difluoropropan-2-yl)-1H-benzo[d][1,2,3]triazol-6-yl)-4-methoxypyrrolo[2,1-f][1,2,4]triazin-2-amine 1499.

White solid (6 mg, 0.011 mmol, 10.5% yield). ¹H NMR (499 MHz, DMSO-d₆) δ ppm 1.76-1.86 (1H, m), 1.86-1.93 (1H, m), 2.16 (1H, br t, J=10.54 Hz), 2.40 (1H, dd, J=27.15, 11.50 Hz), 2.76 (1H, br d, J=11.77 Hz), 2.98-3.07 (1H, m), 3.60 (1H, quin, J=6.30 Hz), 3.97 (3H, s), 4.24-4.37 (1H, m), 4.44 (2H, dt, J=14.85, 6.26 Hz), 4.55 (2H, td, J=6.57, 3.56 Hz), 5.02-5.23 (4H, m), 5.72-5.87 (1H, m), 6.78 (1H, d, J=2.74 Hz), 6.91 (1H, d, J=9.58 Hz), 7.62-7.67 (2H, m), 8.05 (1H, d, J=8.49 Hz), 8.09 (1H, s); ESIMS found for C₂₄H₂₆F₄N₈O₂ m/z 535.25 (M+1).

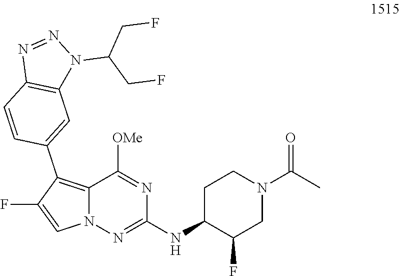 [SMILES: COc1nc(N[C@H]2CCN(C(C)=O)C[C@H]2F)nn2cc(F)c(-c3ccc4nnn(C(CF)CF)c4c3)c12]

1-((3R,4S)-4-((5-(1-(1,3-Difluoropropan-2-yl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one 1515.

White solid (4 mg, 0.008 mmol, 24.5% yield). ¹H NMR (499 MHz, DMSO-d₆) δ ppm 1.64-1.73 (1H, m), 1.74-1.91 (1H, m), 2.00 (3H, s), 2.68-3.00 (1H, m), 3.16-3.25 (1H, m), 3.84-4.02 (2H, m), 3.91 (3H, s), 4.03-4.19 (1H, m), 4.40-4.77 (1H, m), 4.97 (1H, d, J=49.90 Hz), 5.03-5.12 (2H, m), 5.12-5.21 (2H, m), 5.73-5.89 (1H, m), 6.92 (1H, dd, J=7.80, 4.24 Hz), 7.57 (1H, d, J=8.49 Hz), 7.83 (1H, d, J=2.74 Hz), 8.07 (1H, s), 8.10 (1H, d, J=8.76 Hz); ESIMS found for C₂₃H₂₄F₄N₈O₂ m/z 521.2 (M+1).

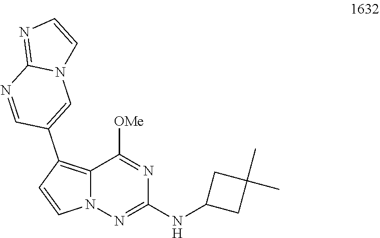 [SMILES: COc1nc(NC2CC(C)(C)C2)nn2ccc(-c3cnc4nccn4c3)c12]

N-(3,3-Dimethylcyclobutyl)-5-(imidazo[1,2-a]pyrimidin-6-yl)-4-methoxypyrrolo[2,1-f][1,2,4]triazin-2-amine 1632.

Off-white solid (17 mg, 0.047 mmol, 28.1% yield). ¹H NMR (499 MHz, DMSO-d₆) δ ppm 1.11 (3H, s), 1.16 (3H, s), 1.76-1.87 (2H, m), 2.09-2.20 (2H, m), 3.97 (3H, s), 4.17 (1H, sxt, J=7.94 Hz), 6.77 (1H, d, J=2.46 Hz), 6.98 (1H, d, J=7.39 Hz), 7.67 (1H, d, J=2.46 Hz), 7.72 (1H, d, J=1.37 Hz), 7.92 (1H, d, J=1.37 Hz), 8.73 (1H, d, J=2.46 Hz), 9.08 (1H, d, J=2.46 Hz); ESIMS found for C₁₉H₂₁N₇O m/z 364.2 (M+1).

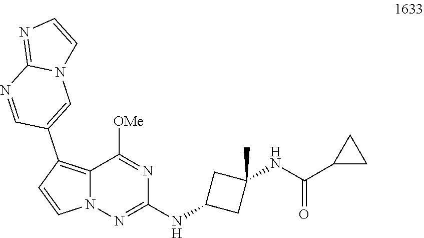 [SMILES: COc1nc(N[C@H]2C[C@@](C)(NC(=O)C3CC3)C2)nn2ccc(-c3cnc4nccn4c3)c12]

N-((1s,3s)-3-((5-(Imidazo[1,2-a]pyrimidin-6-yl)-4-methoxypyrrolo[2,1-f][1,2,4]triazin-2-yl)amino)-1-methylcyclobutyl)cyclopropanecarboxamide 1633.

Off-white solid (11 mg, 0.025 mmol, 92.7% yield). ¹H NMR (499 MHz, DMSO-d₆) δ ppm 0.53-0.67 (4H, m), 1.38 (3H, s), 1.44-1.57 (1H, m), 2.11-2.24 (2H, m), 2.42 (2H, ddd, J=9.58, 7.39, 2.74 Hz), 3.97 (3H, s), 4.03 (1H, dq, J=15.37, 7.93 Hz), 6.78 (1H, d, J=2.46 Hz), 7.04 (1H, d, J=7.12 Hz), 7.68 (1H, d, J=2.46 Hz), 7.72 (1H, d, J=1.37 Hz), 7.93 (1H, d, J=1.37 Hz), 8.23 (1H, s), 8.73 (1H, d, J=2.46 Hz), 9.09 (1H, d, J=2.46 Hz); ESIMS found for C₂₂H₂₄N₈O₂ m/z 433.2 (M+1).

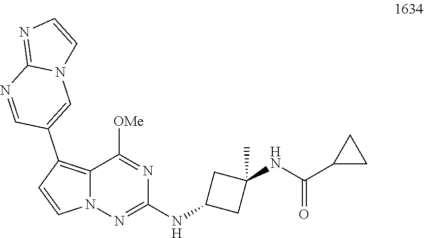 [SMILES: COc1nc(N[C@H]2C[C@](C)(NC(=O)C3CC3)C2)nn2ccc(-c3cnc4nccn4c3)c12]

N-((1r,3r)-3-((5-(Imidazo[1,2-a]pyrimidin-6-yl)-4-methoxypyrrolo[2,1-f][1,2,4]triazin-2-yl)amino)-1-methylcyclobutyl)cyclopropanecarboxamide 1634.

Off-white solid (11 mg, 0.025 mmol, 92.7% yield). ¹H NMR (499 MHz, DMSO-d₆) δ ppm 0.57-0.70 (4H, m), 1.36 (3H, s), 1.54-1.64 (1H, m), 1.90-2.02 (2H, m), 2.60-2.70 (2H, m), 3.97 (3H, s), 4.21 (1H, sxt, J=7.83 Hz), 6.77 (1H, d, J=2.46 Hz), 7.06 (1H, d, J=7.39 Hz), 7.67 (1H, d, J=2.46 Hz), 7.72 (1H, d, J=1.37 Hz), 7.93 (1H, d, J=1.37 Hz), 8.15 (1H, s), 8.73 (1H, d, J=2.46 Hz), 9.08 (1H, d, J=2.46 Hz); ESIMS found for C₂₂H₂₄N₈O₂ m/z 433.2 (M+1).

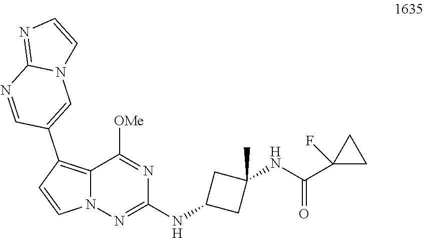 [SMILES: COc1nc(N[C@H]2C[C@@](C)(NC(=O)C3(F)CC3)C2)nn2ccc(-c3cnc4nccn4c3)c12]

1-Fluoro-N-((1s,3s)-3-((5-(imidazo[1,2-a]pyrimidin-6-yl)-4-methoxypyrrolo[2,1-f][1,2,4]triazin-2-yl)amino)-1-methylcyclobutyl)cyclopropane-1-carboxamide 1635.

Beige solid (15 mg, 0.033 mmol, 80.9% yield). ¹H NMR (499 MHz, DMSO-d₆) δ ppm 1.11-1.19 (2H, m), 1.20-1.28 (2H, m), 1.42 (3H, s), 2.23 (2H, br dd, J=11.77, 9.03 Hz), 2.44-2.49 (2H, m), 3.97 (3H, s), 4.00-4.10 (1H, m), 6.78 (1H, d, J=2.74 Hz), 7.06 (1H, d, J=7.12 Hz), 7.68 (1H, d, J=2.74 Hz), 7.72 (1H, d, J=1.37 Hz), 7.93 (1H, d, J=1.37 Hz), 8.48 (1H, s), 8.73 (1H, d, J=2.46 Hz), 9.09 (1H, d, J=2.46 Hz); ESIMS found for C₂₂H₂₃FN₈O₂ m/z 451.2 (M+1).

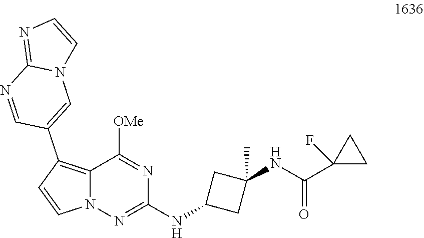 [SMILES: COc1nc(N[C@H]2C[C@](C)(NC(=O)C3(F)CC3)C2)nn2ccc(-c3cnc4nccn4c3)c12]

1-Fluoro-N-((1r,3r)-3-((5-(imidazo[1,2-a]pyrimidin-6-yl)-4-methoxypyrrolo[2,1-f][1,2,4]triazin-2-yl)amino)-1-methylcyclobutyl)cyclopropane-1-carboxamide 1636.

Off-white solid (7 mg, 0.016 mmol, 56.6% yield). ¹H NMR (499 MHz, DMSO-d₆) δ ppm 1.15-1.21 (2H, m), 1.22-1.27 (1H, m), 1.27-1.32 (1H, m), 1.41 (3H, s), 1.96-2.07 (2H, m), 2.74-2.85 (2H, m), 3.97 (3H, s), 4.19 (1H, sxt, J=7.67 Hz), 6.77 (1H, d, J=2.46 Hz), 7.07 (1H, d, J=6.84 Hz), 7.68 (1H, d, J=2.74 Hz), 7.72 (1H, d, J=1.37 Hz), 7.93 (1H, d, J=1.37 Hz), 8.27 (1H, s), 8.73 (1H, d, J=2.46 Hz), 9.08 (1H, d, J=2.74 Hz); ESIMS found for C₂₂H₂₃FN₈O₂ m/z 451.2 (M+1).

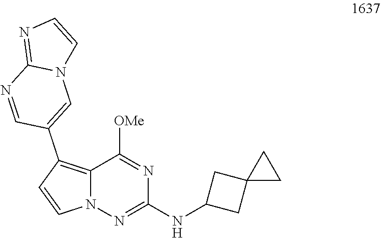 [SMILES: COc1nc(NC2CC3(CC3)C2)nn2ccc(-c3cnc4nccn4c3)c12]

5-(Imidazo[1,2-a]pyrimidin-6-yl)-4-methoxy-N-(spiro[2.3]hexan-5-yl) pyrrolo[2,1-f][1,2,4]triazin-2-amine 1637.

Off-white solid (15 mg, 0.042 mmol, 37.8% yield). ¹H NMR (499 MHz, DMSO-d₆) δ ppm 0.37-0.43 (2H, m), 0.45-0.51 (2H, m), 2.22-2.36 (4H, m), 3.98 (3H, s), 4.37 (1H, sxt, J=7.50 Hz), 6.78 (1H, d, J=2.46 Hz), 7.16 (1H, d, J=7.12 Hz), 7.67 (1H, d, J=2.46 Hz), 7.72 (1H, d, J=1.37 Hz), 7.93 (1H, d, J=1.37 Hz), 8.73 (1H, d, J=2.46 Hz), 9.09 (1H, d, J=2.46 Hz); ESIMS found for C₁₉H₁₉N₇O m/z 362.2 (M+1).

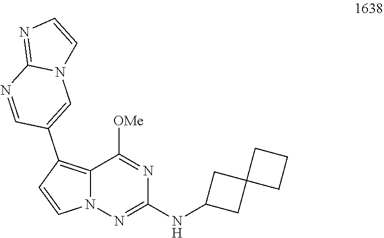 [SMILES: COc1nc(NC2CC3(CCC3)C2)nn2ccc(-c3cnc4nccn4c3)c12]

5-(Imidazo[1,2-a]pyrimidin-6-yl)-4-methoxy-N-(spiro[3.3]heptan-2-yl) pyrrolo[2,1-f][1,2,4]triazin-2-amine 1638.

Off-white solid (43 mg, 0.115 mmol, 57.4% yield). ¹H NMR (499 MHz, DMSO-d₆) δ ppm ¹H NMR (499 MHz, DMSO-d₆) δ ppm 1.76-1.85 (2H, m), 1.87-1.99 (4H, m), 2.04 (2H, t, J=7.12 Hz), 2.34-2.42 (2H, m), 3.96 (3H, s), 4.04 (1H, sxt, J=7.78 Hz), 6.77 (1H, d, J=2.46 Hz), 6.97 (1H, d, J=7.12 Hz), 7.67 (1H, d, J=2.46 Hz), 7.72 (1H, d, J=1.37 Hz), 7.92 (1H, d, J=1.37 Hz), 8.72 (1H, d, J=2.46 Hz), 9.08 (1H, d, J=2.46 Hz); ESIMS found for C₂₀H₂₁N₇O m/z 376.2 (M+1).

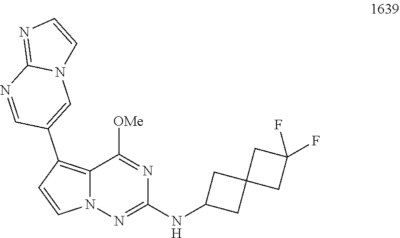 [SMILES: COc1nc(NC2CC3(C2)CC(F)(F)C3)nn2ccc(-c3cnc4nccn4c3)c12]

N-(6,6-Difluorospiro[3.3]heptan-2-yl)-5-(imidazo[1,2-a]pyrimidin-6-yl)-4-methoxypyrrolo[2,1-f][1,2,4]triazin-2-amine 1639.

Off-white solid (30 mg, 0.073 mmol, 36.5% yield). ¹H NMR (499 MHz, DMSO-d₆) δ ppm 2.11-2.22 (2H, m), 2.43-2.49 (2H, m), 2.57 (2H, br t, J=12.59 Hz), 2.69 (2H, br t, J=12.73 Hz), 3.97 (3H, s), 4.14 (1H, sxt, J=7.78 Hz), 6.78 (1H, d, J=2.46 Hz), 7.08 (1H, d, J=7.12 Hz), 7.67 (1H, d, J=2.46 Hz), 7.72 (1H, d, J=1.10 Hz), 7.92 (1H, d, J=1.10 Hz), 8.73 (1H, d, J=2.46 Hz), 9.08 (1H, d, J=2.19 Hz); ESIMS found for C₂₀H₁₉F₂N₇O m/z 412.15 (M+1).

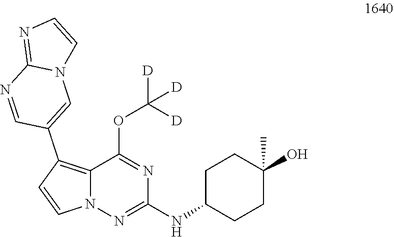 [SMILES: [2H]C([2H])([2H])Oc1nc(N[C@H]2CC[C@](C)(O)CC2)nn2ccc(-c3cnc4nccn4c3)c12]

(1r,4r)-4-((5-(Imidazo[1,2-a]pyrimidin-6-yl)-4-(methoxy-d₃)pyrrolo[2,1-f][1,2,4]triazin-2-yl)amino)-1-methylcyclohexan-1-ol 1640.

Off-white solid (15 mg, 0.038 mmol, 32.1% yield). ¹H NMR (499 MHz, DMSO-d₆) δ ppm 1.14 (3H, s), 1.36-1.52 (4H, m), 1.54-1.68 (2H, m), 1.81-1.93 (2H, m), 3.60-3.70 (1H, m), 4.23 (1H, s), 6.54 (1H, d, J=7.94 Hz), 6.76 (1H, d, J=2.74 Hz), 7.67 (1H, d, J=2.74 Hz), 7.72 (1H, d, J=1.37 Hz), 7.92 (1H, d, J=1.37 Hz), 8.73 (1H, d, J=2.46 Hz), 9.08 (1H, d, J=2.46 Hz); ESIMS found for C₂₀H₂₀[2H₃]N₇O₂ m/z 397.2 (M+1).

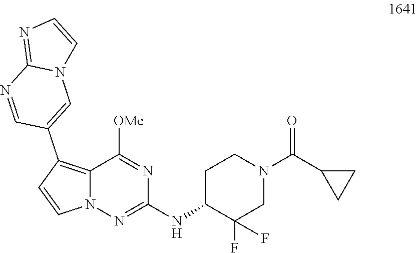 [SMILES: COc1nc(N[C@@H]2CCN(C(=O)C3CC3)CC2(F)F)nn2ccc(-c3cnc4nccn4c3)c12]

(R)-Cyclopropyl(3,3-difluoro-4-((5-(imidazo[1,2-a]pyrimidin-6-yl)-4-methoxypyrrolo[2,1-f][1,2,4]triazin-2-yl)amino)piperidin-1-yl)methanone 1641.

Beige solid (8 mg, 0.017 mmol, 48.8% yield). ¹H NMR (499 MHz, DMSO-d₆) δ ppm 0.67-0.83 (4H, m), 1.57-1.84 (1H, m), 1.86-2.03 (1H, m), 2.07 (1H, quin, J=6.23 Hz), 2.93-3.08 (1H, m), 3.42-3.54 (1H, m), 4.02 (3H, s), 4.27 (1H, br d, J=13.69 Hz), 4.48-4.65 (2H, m), 6.82 (1H, d, J=2.46 Hz), 7.06 (1H, br d, J=9.03 Hz), 7.69 (1H, d, J=2.74 Hz), 7.73 (1H, s), 7.94 (1H, s), 8.75 (1H, d, J=2.19 Hz), 9.11 (1H, d, J=2.46 Hz); ESIMS found for C₂₂H₂₂F₂N₈O₂ m/z 469.2 (M+1).

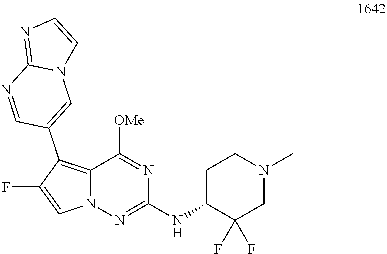 [SMILES: COc1nc(N[C@@H]2CCN(C)CC2(F)F)nn2cc(F)c(-c3cnc4nccn4c3)c12]

(R)—N-(3,3-Difluoro-1-methylpiperidin-4-yl)-6-fluoro-5-(imidazo[1,2-a]pyrimidin-6-yl)-4-methoxypyrrolo[2,1-f][1,2,4]triazin-2-amine 1642.

Fluffy white solid (0.33 mg, 0.0008 mmol, 16.0% yield). ¹H NMR (499 MHz, DMSO-d₆) δ ppm 1.76-1.90 (2H, m), 2.16 (1H, td, J=10.75, 2.87 Hz), 2.25 (3H, s), 2.30-2.45 (1H, m), 2.73-2.84 (1H, m), 2.98-3.08 (1H, m), 3.97 (3H, s), 4.14-4.30 (1H, m), 7.01 (1H, d, J=9.31 Hz), 7.75 (1H, d, J=1.37 Hz), 7.89 (1H, d, J=2.74 Hz), 7.96 (1H, d, J=1.37 Hz), 8.65 (1H, d, J=2.19 Hz), 9.12 (1H, d, J=2.46 Hz); ESIMS found for C₁₉H₁₉F₃N₈O m/z 433.2 (M+1).

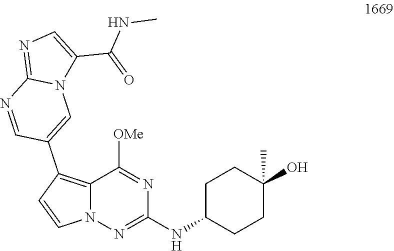 [SMILES: CNC(=O)c1cnc2ncc(-c3ccn4nc(N[C@H]5CC[C@](C)(O)CC5)nc(OC)c34)cn12]

6-(2-(((1r,4r)-4-Hydroxy-4-methylcyclohexyl)amino)-4-methoxypyrrolo[2,1-f][1,2,4]triazin-5-yl)-N-methylimidazo[1,2-a]pyrimidine-3-carboxamide 1669.

Brown solid (20 mg, 0.044 mmol, 34.5% yield). ¹H NMR (499 MHz, DMSO-d₆) δ ppm 1.14 (3H, s), 1.36-1.51 (4H, m), 1.55-1.65 (2H, m), 1.80-1.94 (2H, m), 2.84 (3H, d, J=4.65 Hz), 3.65 (1H, dt, J=7.87, 4.14 Hz), 4.03 (3H, s), 4.23 (1H, s), 6.58 (1H, d, J=7.94 Hz), 6.90 (1H, d, J=2.74 Hz), 7.69 (1H, d, J=2.74 Hz), 8.40 (1H, s), 8.58 (1H, q, J=4.38 Hz), 8.95 (1H, d, J=2.46 Hz), 10.05 (1H, d, J=2.46 Hz); ESIMS found for C₂₂H₂₆N₈O₃ m/z 451.25 (M+1).

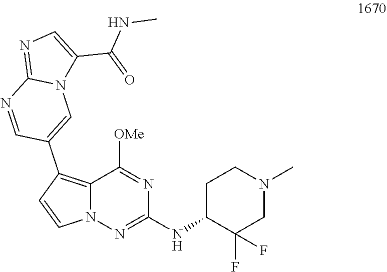 [SMILES: CNC(=O)c1cnc2ncc(-c3ccn4nc(N[C@@H]5CCN(C)CC5(F)F)nc(OC)c34)cn12]

(R)-6-(2-((3,3-Difluoro-1-methylpiperidin-4-yl)amino)-4-methoxypyrrolo [2,1-f][1,2,4]triazin-5-yl)-N-methylimidazo[1,2-a]pyrimidine-3-carboxamide 1670.

White solid (8 mg, 0.017 mmol, 22.5% yield). ¹H NMR (499 MHz, DMSO-d₆) δ ppm 1.75-1.89 (2H, m), 2.12-2.23 (1H, m), 2.26 (3H, s), 2.38 (1H, dd, J=25.50, 12.87 Hz), 2.75-2.81 (1H, m), 2.84 (3H, d, J=4.65 Hz), 2.99-3.12 (1H, m), 4.06 (3H, s), 4.16-4.33 (1H, m), 6.90-6.97 (2H, m), 7.70 (1H, d, J=2.74 Hz), 8.41 (1H, s), 8.54-8.67 (1H, m), 8.96 (1H, d, J=2.46 Hz), 10.06 (1H, d, J=2.46 Hz); ESIMS found for C₂₁H₂₃F₂N₉O₂ m/z 472.2 (M+1).

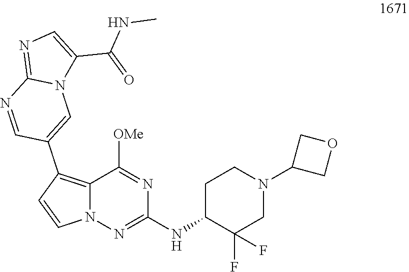 [SMILES: CNC(=O)c1cnc2ncc(-c3ccn4nc(N[C@@H]5CCN(C6COC6)CC5(F)F)nc(OC)c34)cn12]

(R)-6-(2-((3,3-Difluoro-1-(oxetan-3-yl)piperidin-4-yl)amino)-4-methoxypyrrolo[2,1-f][1,2,4]triazin-5-yl)-N-methylimidazo[1,2-a]pyrimidine-3-carboxamide 1671.

Off-white solid (14 mg, 0.027 mmol, 46.3% yield). ¹H NMR (499 MHz, DMSO-d₆) δ ppm 1.75-1.86 (1H, m), 1.87-1.94 (1H, m), 2.12-2.21 (1H, m), 2.40 (1H, dd, J=27.45, 12.32 Hz), 2.76 (1H, br d, J=11.77 Hz), 2.84 (3H, d, J=4.65 Hz), 2.95-3.08 (1H, m), 3.60 (1H, quin, J=6.30 Hz), 4.07 (3H, s), 4.24-4.37 (1H, m), 4.44 (2H, dt, J=15.06, 6.16 Hz), 4.55 (2H, td, J=6.57, 3.56 Hz), 6.95 (1H, d, J=2.74 Hz), 7.00 (1H, d, J=9.31 Hz), 7.70 (1H, d, J=2.74 Hz), 8.41 (1H, s), 8.59 (1H, q, J=4.56 Hz), 8.96 (1H, d, J=2.74 Hz), 10.06 (1H, d, J=2.74 Hz); ESIMS found for C₂₃H₂₅F₂N₉O₃ m/z 514.2 (M+1).

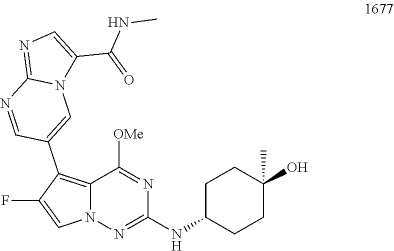 [SMILES: CNC(=O)c1cnc2ncc(-c3c(F)cn4nc(N[C@H]5CC[C@](C)(O)CC5)nc(OC)c34)cn12]

6-(6-Fluoro-2-(((1r,4r)-4-hydroxy-4-methylcyclohexyl)amino)-4-methoxypyrrolo[2,1-f][1,2,4]triazin-5-yl)-N-methylimidazo[1,2-a]pyrimidine-3-carboxamide 1677.

Colorless semi-solid (0.46 mg, 0.001 mmol, 3.7% yield). ¹H NMR (499 MHz, DMSO-d₆) δ ppm 1.14 (3H, s), 1.34-1.52 (4H, m), 1.56-1.66 (2H, m), 1.80-1.94 (2H, m), 2.83 (3H, d, J=4.65 Hz), 3.64 (1H, td, J=8.28, 4.24 Hz), 3.97 (3H, s), 4.24 (1H, s), 6.69 (1H, d, J=7.94 Hz), 7.91 (1H, d, J=2.74 Hz), 8.44 (1H, s), 8.63 (1H, q, J=4.47 Hz), 8.85 (1H, d, J=2.46 Hz), 9.91 (1H, d, J=2.46 Hz); ESIMS found for C₂₂H₂₅FN₈O₃ m/z 469.2 (M+1).

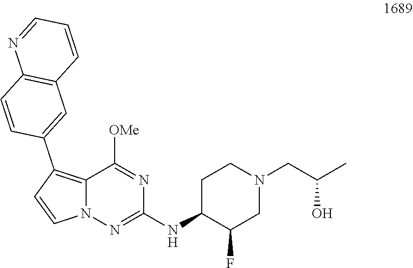 [SMILES: COc1nc(N[C@H]2CCN(C[C@H](C)O)C[C@H]2F)nn2ccc(-c3ccc4ncccc4c3)c12]

(S)-1-((3R,4S)-3-Fluoro-4-((4-methoxy-5-(quinolin-6-yl)pyrrolo[2,1-f][1,2,4]triazin-2-yl)amino)piperidin-1-yl)propan-2-ol 1689.

White solid (15 mg, 0.033 mmol, 32.7% yield). ¹H NMR (499 MHz, DMSO-d₆) δ ppm 1.05 (3H, d, J=6.30 Hz), 1.64-1.74 (1H, m), 1.93 (1H, qd, J=12.32, 3.83 Hz), 2.15-2.38 (4H, m), 2.90 (1H, br d, J=10.40 Hz), 3.12-3.22 (1H, m), 3.71-3.89 (2H, m), 3.95-4.02 (3H, m), 4.30 (1H, d, J=4.11 Hz), 4.90 (1H, d, J=49.65 Hz), 6.62 (1H, d, J=7.94 Hz), 6.79 (1H, d, J=2.74 Hz), 7.53 (1H, dd, J=8.21, 4.11 Hz), 7.65 (1H, d, J=2.74 Hz), 7.95-8.04 (2H, m), 8.11 (1H, d, J=1.37 Hz), 8.36 (1H, dd, J=8.21, 1.64 Hz), 8.86 (1H, dd, J=4.11, 1.64 Hz); ESIMS found for C₂₄H₂₇FN₆O₂ m/z 226.2 (M/2+1).

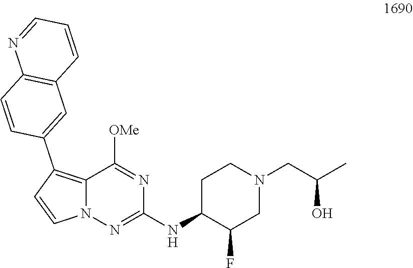 [SMILES: COc1nc(N[C@H]2CCN(C[C@@H](C)O)C[C@H]2F)nn2ccc(-c3ccc4ncccc4c3)c12]

(R)-1-((3R,4S)-3-Fluoro-4-((4-methoxy-5-(quinolin-6-yl)pyrrolo[2,1-f][1,2,4]triazin-2-yl)amino)piperidin-1-yl)propan-2-ol 1690.

White solid (15 mg, 0.033 mmol, 32.7% yield). ¹H NMR (499 MHz, DMSO-d₆) δ ppm 1.05 (3H, d, J=6.30 Hz), 1.65-1.74 (1H, m), 1.87-1.97 (1H, m), 2.17-2.25 (2H, m), 2.27-2.33 (1H, m), 2.36 (1H, dd, J=37.85, 12.87 Hz), 2.87-2.96 (1H, m), 3.08-3.21 (1H, m), 3.71-3.86 (2H, m), 3.94-4.01 (3H, m), 4.27 (1H, d, J=3.83 Hz), 4.90 (1H, d, J=50.20 Hz), 6.63 (1H, d, J=7.94 Hz), 6.79 (1H, d, J=2.74 Hz), 7.53 (1H, dd, J=8.21, 4.11 Hz), 7.65 (1H, d, J=2.74 Hz), 7.94-8.04 (2H, m), 8.11 (1H, s), 8.36 (1H, dd, J=8.21, 1.64 Hz), 8.86 (1H, dd, J=4.11, 1.64 Hz); ESIMS found for C₂₄H₂₇FN₆O₂ m/z 226.2 (M/2+1).

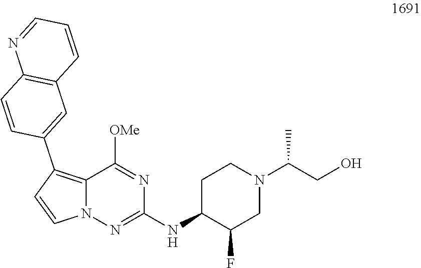 [SMILES: COc1nc(N[C@H]2CCN([C@H](C)CO)C[C@H]2F)nn2ccc(-c3ccc4ncccc4c3)c12]

(R)-2-((3R,4S)-3-Fluoro-4-((4-methoxy-5-(quinolin-6-yl)pyrrolo[2,1-f][1,2,4]triazin-2-yl)amino)piperidin-1-yl)propan-1-ol 1691.

White solid (6 mg, 0.013 mmol, 13.1% yield). ¹H NMR (499 MHz, DMSO-d₆) δ ppm 1.05 (3H, d, J=6.02 Hz), 1.63-1.74 (1H, m), 1.92 (1H, qd, J=12.23, 3.83 Hz), 2.17-2.25 (2H, m), 2.27-2.33 (1H, m), 2.36 (1H, dd, J=37.55, 12.59 Hz), 2.91 (1H, br d, J=11.77 Hz), 3.15 (1H, br t, J=10.54 Hz), 3.72-3.86 (1H, m), 3.76 (1H, sxt, J=6.30 Hz), 3.98 (3H, s), 4.28 (1H, br s), 4.90 (1H, d, J=49.65 Hz), 6.62 (1H, d, J=7.67 Hz), 6.79 (1H, d, J=2.46 Hz), 7.53 (1H, dd, J=8.21, 4.11 Hz), 7.65 (1H, d, J=2.46 Hz), 7.95-8.03 (2H, m), 8.11 (1H, s), 8.36 (1H, dd, J=8.21, 1.37 Hz), 8.86 (1H, dd, J=4.38, 1.64 Hz); ESIMS found for C₂₄H₂₇FN₆O₂ m/z 226.2 (M/2+1).

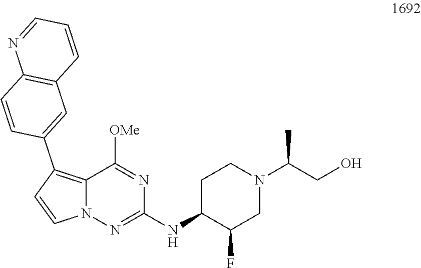 [SMILES: COc1nc(N[C@H]2CCN([C@@H](C)CO)C[C@H]2F)nn2ccc(-c3ccc4ncccc4c3)c12]

(S)-2-((3R,4S)-3-Fluoro-4-((4-methoxy-5-(quinolin-6-yl)pyrrolo[2,1-f][1,2,4]triazin-2-yl)amino)piperidin-1-yl)propan-1-ol 1692.

White solid (15 mg, 0.033 mmol, 32.7% yield). ¹H NMR (499 MHz, DMSO-d₆) δ ppm 1.05 (3H, d, J=6.02 Hz), 1.69 (1H, br dd, J=12.73, 2.33 Hz), 1.87-1.99 (1H, m), 2.16-2.37 (4H, m), 2.90 (1H, br d, J=11.77 Hz), 3.12-3.21 (1H, m), 3.72-3.86 (1H, m), 3.76 (1H, sxt, J=6.30 Hz), 3.98 (3H, s), 4.30 (1H, br s), 4.90 (1H, d, J=50.45 Hz), 6.62 (1H, d, J=7.94 Hz), 6.79 (1H, d, J=2.74 Hz), 7.53 (1H, dd, J=8.21, 4.11 Hz), 7.65 (1H, d, J=2.46 Hz), 7.95-8.04 (2H, m), 8.11 (1H, s), 8.36 (1H, dd, J=8.35, 1.51 Hz), 8.86 (1H, dd, J=4.11, 1.64 Hz); ESIMS found for C₂₄H₂₇FN₆O₂ m/z 226.15 (M/2+1).

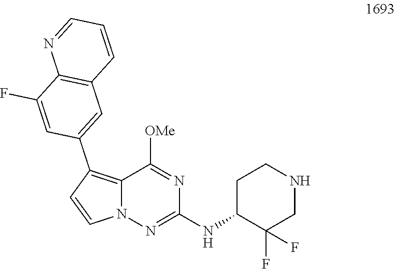 [SMILES: COc1nc(N[C@@H]2CCNCC2(F)F)nn2ccc(-c3cc(F)c4ncccc4c3)c12]

(R)—N-(3,3-Difluoropiperidin-4-yl)-5-(8-fluoroquinolin-6-yl)-4-methoxypyrrolo[2,1-f][1,2,4]triazin-2-amine 1693.

White solid (141 mg, 0.329 mmol, 98.3% yield). ¹H NMR (500 MHz, DMSO-d₆) δ ppm 1.71 (1H, qd, J=12.12, 3.70 Hz), 1.81-1.93 (1H, m), 2.62 (1H, br t, J=11.53 Hz), 2.84 (1H, dd, J=29.40, 13.72 Hz), 2.94 (1H, br d, J=11.80 Hz), 3.09-3.21 (1H, m), 4.02 (3H, s), 4.28-4.44 (1H, m), 6.85 (1H, d, J=2.47 Hz), 6.91 (1H, d, J=9.33 Hz), 7.63 (1H, dd, J=8.51, 4.12 Hz), 7.67 (1H, d, J=2.74 Hz), 7.85 (1H, dd, J=12.62, 1.65 Hz), 7.99 (1H, d, J=1.65 Hz), 8.44 (1H, d, J=8.51 Hz), 8.91 (1H, dd, J=4.12, 1.65 Hz); ESIMS found for C₂₁H₁₉F₃N₆O m/z 429.15 (M+1).

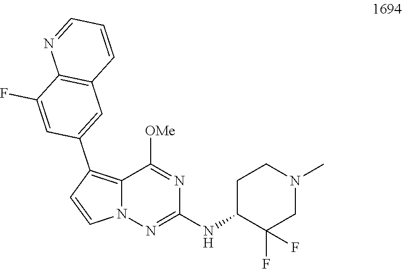 [SMILES: COc1nc(N[C@@H]2CCN(C)CC2(F)F)nn2ccc(-c3cc(F)c4ncccc4c3)c12]

(R)—N-(3,3-Difluoro-1-methylpiperidin-4-yl)-5-(8-fluoroquinolin-6-yl)-4-methoxypyrrolo[2,1-f][1,2,4]triazin-2-amine 1694.

White solid (20 mg, 0.045 mmol, 43.0% yield). ¹H NMR (500 MHz, DMSO-d₆) δ ppm 1.77-1.90 (2H, m), 2.18 (1H, td, J=11.05, 3.16 Hz), 2.26 (3H, s), 2.38 (1H, dd, J=27.70, 11.80 Hz), 2.79 (1H, br d, J=11.53 Hz), 2.98-3.13 (1H, m), 4.02 (3H, s), 4.17-4.32 (1H, m), 6.86 (1H, d, J=2.74 Hz), 6.88 (1H, d, J=9.61 Hz), 7.63 (1H, dd, J=8.37, 4.25 Hz), 7.67 (1H, d, J=2.74 Hz), 7.85 (1H, dd, J=12.49, 1.78 Hz), 7.99 (1H, d, J=1.65 Hz), 8.44 (1H, d, J=8.23 Hz), 8.91 (1H, dd, J=4.12, 1.65 Hz); ESIMS found for C₂₂H₂₁F₃N₆O m/z 443.2 (M+1).

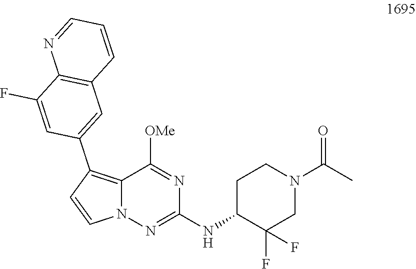 [SMILES: COc1nc(N[C@@H]2CCN(C(C)=O)CC2(F)F)nn2ccc(-c3cc(F)c4ncccc4c3)c12]

(R)-1-(3,3-Difluoro-4-((5-(8-fluoroquinolin-6-yl)-4-methoxypyrrolo[2,1-f][1,2,4]triazin-2-yl)amino)piperidin-1-yl)ethan-1-one 1695.

Off-white solid (23 mg, 0.049 mmol, 46.5% yield). ¹H NMR (500 MHz, DMSO-d₆) δ ppm 1.58-1.85 (1H, m), 1.85-1.98 (1H, m), 2.01-2.09 (3H, m), 2.93-3.05 (1H, m), 3.61-3.93 (1H, m), 4.03 (3H, s), 4.10-4.34 (1H, m), 4.44-4.62 (2H, m), 6.87 (1H, d, J=2.74 Hz), 7.04 (1H, d, J=9.33 Hz), 7.63 (1H, dd, J=8.23, 4.12 Hz), 7.67 (1H, dd, J=2.47, 1.37 Hz), 7.85 (1H, dd, J=12.62, 1.65 Hz), 7.99 (1H, d, J=1.65 Hz), 8.44 (1H, d, J=8.51 Hz), 8.91 (1H, dd, J=4.12, 1.37 Hz); ESIMS found for C₂₃H₂₁F₃N₆O₂ m/z 471.1 (M+1).

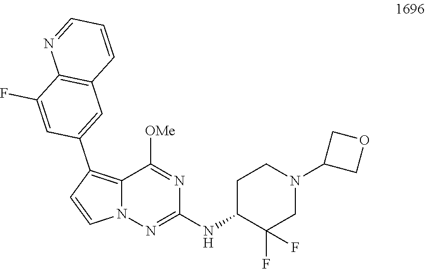 [SMILES: COc1nc(N[C@@H]2CCN(C3COC3)CC2(F)F)nn2ccc(-c3cc(F)c4ncccc4c3)c12]

(R)—N-(3,3-Difluoro-1-(oxetan-3-yl)piperidin-4-yl)-5-(8-fluoroquinolin-6-yl)-4-methoxypyrrolo[2,1-f][1,2,4]triazin-2-amine 1696.

White solid (33 mg, 0.068 mmol, 64.8% yield). ¹H NMR (500 MHz, DMSO-d₆) δ ppm 1.77-1.87 (1H, m), 1.87-1.93 (1H, m), 2.16 (1H, br t, J=10.57 Hz), 2.41 (1H, dd, J=27.00, 11.53 Hz), 2.76 (1H, br d, J=11.53 Hz), 2.96-3.08 (1H, m), 3.60 (1H, quin, J=6.17 Hz), 4.02 (3H, s), 4.23-4.38 (1H, m), 4.44 (2H, dt, J=15.09, 6.17 Hz), 4.55 (2H, td, J=6.52, 3.71 Hz), 6.86 (1H, d, J=2.47 Hz), 6.94 (1H, d, J=9.33 Hz), 7.63 (1H, dd, J=8.23, 4.12 Hz), 7.67 (1H, d, J=2.47 Hz), 7.85 (1H, dd, J=12.49, 1.51 Hz), 7.99 (1H, d, J=1.37 Hz), 8.44 (1H, d, J=8.51 Hz), 8.91 (1H, dd, J=4.12, 1.37 Hz); ESIMS found for C₂₄H₂₃F₃N₆O₂ m/z 485.2 (M+1).

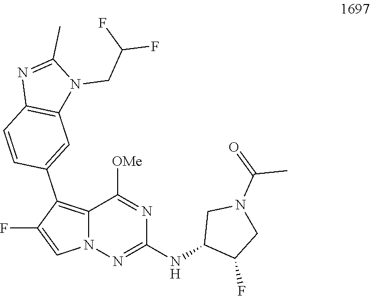 [SMILES: COc1nc(N[C@@H]2CN(C(C)=O)C[C@@H]2F)nn2cc(F)c(-c3ccc4nc(C)n(CC(F)F)c4c3)c12]

1-((3R,4S)-3-((5-(1-(2,2-Difluoroethyl)-2-methyl-1H-benzo[d]imidazol-6-yl)-6-fluoro-4-methoxypyrrolo[2,1-f][1,2,4]triazin-2-yl)amino)-4-fluoropyrrolidin-1-yl)ethan-1-one 1697.

White solid (7.1 mg, 0.014 mmol, 56.6% yield). ¹H NMR (499 MHz, DMSO-d₆) δ ppm 1.97 (3H, d, J=19.16 Hz), 2.58 (3H, s), 3.45-3.54 (1H, m), 3.57-3.75 (1H, m), 3.77-3.84 (1H, m), 3.85-3.96 (3H, m), 3.90-3.92 (1H, m), 4.27-4.56 (1H, m), 4.75 (2H, td, J=16.22, 2.05 Hz), 5.24-5.46 (1H, m), 6.46 (1H, tt, J=54.30, 2.87 Hz), 7.07-7.14 (1H, m), 7.30 (1H, d, J=8.49 Hz), 7.55 (1H, d, J=8.21 Hz), 7.66 (1H, s), 7.77-7.83 (1H, m); ESIMS found for C₂₃H₂₃F₄N₇O₂ m/z 506.2 (M+1).

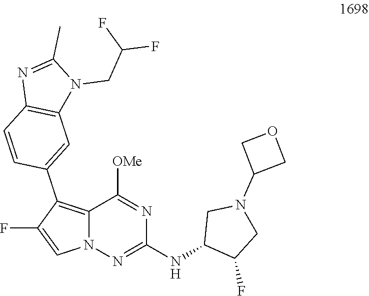 [SMILES: COc1nc(N[C@@H]2CN(C3COC3)C[C@@H]2F)nn2cc(F)c(-c3ccc4nc(C)n(CC(F)F)c4c3)c12]

5-(1-(2,2-Difluoroethyl)-2-methyl-1H-benzo[d]imidazol-6-yl)-6-fluoro-N-((3R,4S)-4-fluoro-1-(oxetan-3-yl)pyrrolidin-3-yl)-4-methoxypyrrolo[2,1-f][1,2,4]triazin-2-amine 1698.

White solid (4.9 mg, 0.009 mmol, 39.2% yield). ¹H NMR (499 MHz, DMSO-d₆) δ ppm 2.58 (3H, s), 2.67-2.92 (2H, m), 2.95-3.09 (1H, m), 3.12-3.22 (1H, m), 3.77-3.87 (1H, m), 3.90 (3H, s), 4.22-4.40 (1H, m), 4.48 (2H, t, J=5.89 Hz), 4.61 (2H, t, J=6.57 Hz), 4.75 (2H, td, J=16.22, 2.33 Hz), 5.25 (1H, d, J=55.95 Hz), 6.46 (1H, tt, J=54.30, 2.87 Hz), 6.81-6.92 (1H, m), 7.30 (1H, d, J=8.21 Hz), 7.55 (1H, d, J=8.21 Hz), 7.65 (1H, s), 7.79 (1H, d, J=3.01 Hz); ESIMS found for C₂₄H₂₅F₄N₇O₂ m/z 520.25 (M+1).

### Example 5

Representative compounds were screened using the assay procedure for DYRK1A kinase activity as described below.

Each compound was dissolved in DMSO as a 10 mM stock and used to prepare compound source plates. Serial dilution (1:3, 11-point dose-response curves from 10 μM to 0.00016 μM) and compound transfer was performed using the ECHO 550 (Labcyte, Sunnyvale, CA) into 1536-well black-walled round bottom plates (Corning).

The DYRK1A kinase assay was run using the Ser/Thr 18 peptide Z-lyte assay kit according to manufacturer's instructions (Life Technologies—a Division of Thermo-Fisher). This is a non-radioactive assay using fluorescence resonance energy transfer (FRET) between coumarin and fluorescein to detect kinase activity which is represented as a ratio of coumarin emission/fluorescein emission.

Briefly, recombinant DYRK1A kinase, ATP and Ser/Thr peptide 18 were prepared in 1×Kinase buffer to final concentrations of 0.25 μg/mL, 15 μM, and 4 μM respectively. The mixture was allowed to incubate with the representative compounds for one hour at room temperature. All reactions were performed in duplicate. Unphosphorylated (“0% Control”) and phosphorylated (“100% control”) forms of Ser/Thr 18 served as control reactions. Additionally, an 11-point dose-response curve of Staurosporine (1 uM top) was run to serve as a positive compound control.

After incubation, Development Reagent A was diluted in Development Buffer then added to the reaction and allowed to further incubate for one hour at room temperature. The plate was read at Ex 400 Em 455 to detect the coumarin signal and Ex 400 Em 520 to measure the signal (EnVision Multilabel Plate Reader, PerkinElmer).

The Emission ratio (Em) was calculated as a ratio ofthe coumarin (C) emission signal (at 445 nm)/Fluorescein (F) emission signal (at 520 nm). The percent phosphorylation was then calculated using the following formula: [1−((Em ratio×F₁₀₀%)−C100%)/((C0%−C100%)+ (Em ratio×(F100%−F0%)))]. Dose-response curves were generated, and inhibitory concentration (IC₅₀) values were calculated using non-linear regression curve fit in the Dotmatics' Studies Software (Bishops Stortford, UK).

Table 3 shows the measured activity for representative compounds of Formula I as described herein.

TABLE 3

| Compound | EC₅₀ (μM) |
|---|---|
| 1 | 0.0016 |
| 2 | 0.0024 |
| 3 | 0.0022 |
| 4 | 0.0047 |
| 5 | 0.0009 |
| 6 | 0.0006 |
| 7 | 0.0010 |
| 9 | 0.0009 |
| 10 | 0.0005 |
| 11 | 0.0012 |
| 12 | 0.0018 |
| 41 | 0.0010 |
| 42 | 0.0006 |
| 44 | 0.0005 |
| 57 | 0.0016 |
| 58 | 0.0020 |
| 76 | 0.0008 |
| 77 | 0.0012 |
| 271 | 0.0024 |
| 299 | 0.0018 |
| 305 | 0.0019 |
| 310 | 0.0037 |
| 313 | 0.0029 |
| 317 | 0.0065 |
| 343 | 0.0011 |
| 468 | 0.0009 |
| 474 | 0.0017 |
| 482 | 0.0038 |
| 535 | 0.0030 |
| 541 | 0.0016 |
| 549 | 0.0025 |
| 553 | 0.0042 |
| 603 | 0.0100 |
| 604 | 0.0068 |
| 605 | 0.0033 |
| 606 | 0.0036 |
| 607 | 0.0018 |
| 608 | 0.0014 |
| 609 | 0.0019 |
| 610 | 0.0011 |
| 611 | 0.0011 |
| 612 | 0.0015 |
| 613 | 0.0004 |
| 616 | 0.0006 |
| 621 | 0.0006 |
| 622 | 0.0011 |
| 623 | 0.0008 |
| 624 | 0.0007 |
| 625 | 0.0015 |
| 626 | 0.0019 |
| 632 | 0.0017 |
| 633 | 0.0009 |
| 635 | 0.0008 |
| 636 | 0.0004 |
| 637 | 0.0003 |
| 686 | 0.0017 |
| 687 | 0.0008 |
| 688 | 0.0014 |
| 689 | 0.0019 |
| 690 | 0.0018 |
| 691 | 0.0016 |
| 699 | 0.0005 |
| 702 | 0.0016 |
| 705 | 0.0008 |
| 710 | 0.0011 |
| 713 | 0.0013 |
| 715 | 0.0042 |
| 716 | 0.0023 |
| 717 | 0.0009 |
| 766 | 0.0017 |
| 772 | 0.0003 |
| 773 | 0.0007 |
| 774 | 0.0010 |
| 775 | 0.0022 |
| 776 | 0.0007 |
| 777 | 0.0013 |
| 778 | 0.0011 |
| 779 | 0.0007 |
| 780 | 0.0015 |
| 787 | 0.0007 |
| 789 | 0.0005 |
| 790 | 0.0005 |
| 791 | 0.0013 |
| 823 | 0.0009 |
| 824 | 0.0012 |
| 825 | 0.0014 |
| 826 | 0.0018 |
| 834 | 0.0006 |
| 841 | 0.0015 |
| 846 | 0.0013 |
| 886 | 0.0056 |
| 887 | 0.0012 |
| 888 | 0.0019 |
| 889 | 0.0017 |
| 890 | 0.0025 |
| 891 | 0.0024 |
| 892 | 0.0011 |
| 893 | 0.0011 |
| 894 | 0.0028 |
| 895 | 0.0007 |
| 899 | 0.0010 |
| 904 | 0.0004 |
| 905 | 0.0011 |
| 906 | 0.0009 |
| 907 | 0.0029 |
| 908 | 0.0013 |
| 909 | 0.0012 |
| 910 | 0.0029 |
| 915 | 0.0008 |
| 917 | 0.0008 |
| 918 | 0.0007 |
| 919 | 0.0009 |
| 920 | 0.0020 |
| 969 | 0.0011 |
| 970 | 0.0017 |
| 981 | 0.0005 |
| 987 | 0.0005 |
| 992 | 0.0021 |
| 995 | 0.0006 |
| 999 | 0.0043 |
| 1025 | 0.0010 |
| 1026 | 0.0003 |
| 1027 | 0.0015 |
| 1044 | 0.0017 |
| 1173 | 0.0011 |
| 1175 | 0.0008 |
| 1182 | 0.0005 |
| 1188 | 0.0018 |
| 1191 | 0.0011 |
| 1195 | 0.0008 |
| 1199 | 0.0010 |
| 1212 | 0.0009 |
| 1215 | 0.0013 |
| 1230 | 0.0019 |
| 1236 | 0.0009 |
| 1237 | 0.0043 |
| 1239 | 0.0012 |
| 1243 | 0.0021 |
| 1269 | 0.0007 |
| 1270 | 0.0036 |
| 1271 | 0.0011 |
| 1272 | 0.0012 |
| 1273 | 0.0018 |
| 1274 | 0.0014 |
| 1290 | 0.0025 |
| 1291 | 0.0007 |
| 1468 | 0.0023 |
| 1470 | 0.0010 |
| 1481 | 0.0008 |
| 1487 | 0.0009 |
| 1495 | 0.0006 |
| 1499 | 0.0021 |
| 1515 | 0.0040 |
| 1632 | 0.0012 |
| 1633 | 0.0006 |
| 1634 | 0.0008 |
| 1635 | 0.0010 |
| 1636 | 0.0003 |
| 1637 | 0.0006 |
| 1638 | 0.0016 |
| 1639 | 0.0013 |
| 1640 | 0.0007 |
| 1641 | 0.0010 |
| 1642 | 0.0028 |
| 1669 | 0.0005 |
| 1670 | 0.0013 |
| 1671 | 0.0008 |
| 1677 | 0.0023 |
| 1689 | 0.0012 |
| 1690 | 0.0021 |
| 1691 | 0.0016 |
| 1692 | 0.0029 |
| 1693 | 0.0019 |
| 1694 | 0.0046 |
| 1695 | 0.0011 |
| 1696 | 0.0017 |
| 1697 | 0.0008 |
| 1698 | 0.0018 |

What is claimed is:

1. A compound, or a pharmaceutically acceptable salt thereof, of Formula I: 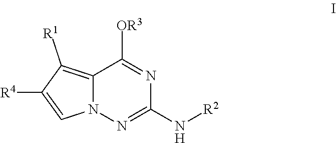
wherein:
R¹ is a 9-10 membered heteroaryl optionally substituted with 1-10 R⁵;
R² is selected from the group consisting of —(C₁₋₅ alkylene)CN, -heterocyclyl optionally substituted with 1-10 R⁶, —(C₁₋₂ alkylene)C(═O)heterocyclyl, and -carbocyclyl optionally substituted with 1-12 R⁷, wherein the —(C₁₋₂ alkylene) or (C₁₋₅ alkylene) is optionally substituted with 1-2 halide and/or 1-2 unsubstituted —(C₁₋₃ alkyl);
R³ is unsubstituted —(C₁₋₃ alkyl);
R⁴ is selected from the group consisting of H and halide;
each R⁵ is independently selected from the group consisting of halide, unsubstituted —(C₁₋₉ alkyl), unsubstituted —(C₁₋₉ haloalkyl), —(C₁₋₄ alkylene)OH, -carbocyclyl optionally substituted with 1-12 halides, —C(═O)N(R⁸)₂, wherein the —(C₁₋₄ alkylene) is optionally substituted with 1-4 halide and/or 1-2 unsubstituted —(C₁₋₃ alkyl);
each R⁶ is independently selected from the group consisting of halide, unsubstituted —(C₁₋₉ alkyl), unsubstituted -heterocyclyl, —(C₁₋₃ alkylene)OR⁹, —SO₂Me, and —(C₁₋₃ alkylene)ₚC(═O)R¹⁰, wherein each —(C₁₋₃ alkylene) is, independently, optionally substituted with 1-2 halide and/or 1-2 unsubstituted —(C₁₋₃ alkyl);
alternatively, two R⁶ attached to the same carbon atom are taken together to form a carbonyl group;
each R⁷ is independently selected from the group consisting of halide, unsubstituted —(C₁₋₉ alkyl), —OH, and —NHC(═O)R¹;
each R⁸ is independently selected from the group consisting of H, unsubstituted —(C₁₋₉ alkyl), and unsubstituted —(C₁₋₉ haloalkyl);
each R⁹ is independently selected from the group consisting of H and unsubstituted —(C₁₋₉ alkyl);
each R¹⁰ is independently selected from the group consisting of unsubstituted —(C₁₋₉ alkyl), —(C₁₋₂ alkylene)ₚN(R¹²)₂, —(C₁₋₂ alkylene)OR⁹, —(C₁₋₂ alkylene)ₚheterocyclyl, and unsubstituted -carbocyclyl, wherein each —(C₁₋₂ alkylene) is, independently, optionally substituted with 1-2 halide and/or 1-2 unsubstituted —(C₁₋₃ alkyl);
each R¹¹ is independently selected from the group consisting of unsubstituted —(C₁₋₉ alkyl), —(C₁₋₂ alkylene)ₚOR⁹, and -carbocyclyl optionally substituted with 1-12 halides, wherein the —(C₁₋₂ alkylene) is optionally substituted with 1-2 halide and/or 1-2 unsubstituted —(C₁₋₃ alkyl);
each R¹² is independently selected from the group consisting of H and unsubstituted —(C₁₋₉ alkyl);
each p is independently 0 or 1; and
wherein each H atom is optionally, independently replaced by ²H (D) (deuterium).

2. The compound of claim 1, wherein R¹ is selected from the group consisting of 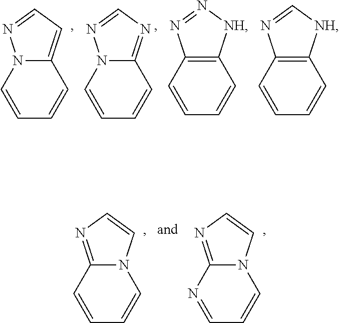
optionally substituted with 1-10 R⁵.

3. The compound of claim 2, wherein R¹ is selected from the group consisting of 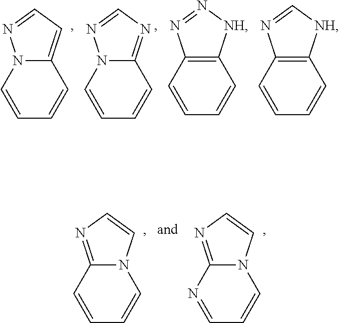
optionally substituted with 1-3 R⁵.

4. The compound of claim 3, wherein R¹ is selected from the group consisting of 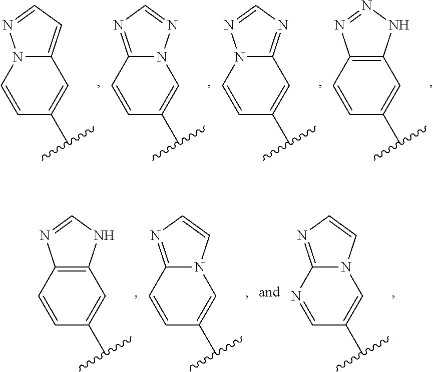
optionally substituted with 1-3 R⁵.

5. The compound of claim 4, wherein R¹ is selected from the group consisting of 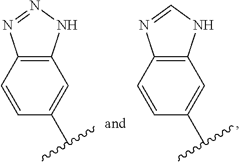 [SMILES: *c1ccc2nn[nH]c2c1 ; *c1ccc2nc[nH]c2c1]
optionally substituted with 1-3 R⁵.

6. The compound of claim 1, wherein each R⁵ is independently selected from the group consisting of F, unsubstituted —(C₁₋₃ alkyl), unsubstituted —(C₁₋₃ haloalkyl), —(C₁₋₃ alkylene)OH, -carbocyclyl optionally substituted with 1-4 fluorines, —C(═O)N(R⁸)₂, wherein the —(C₁₋₃ alkylene) is optionally substituted with 1-2 halide.

7. The compound of claim 5, wherein each R⁵ is independently selected from the group consisting of F, unsubstituted —(C₁₋₃ alkyl), unsubstituted —(C₁₋₃ haloalkyl), —(C₁₋₃ alkylene)OH, -carbocyclyl optionally substituted with 1-4 fluorines, —C(═O)N(R⁸)₂, wherein the —(C₁₋₃ alkylene) is optionally substituted with 1-2 halide.

8. The compound of claim 6, wherein R² is selected from the group consisting of —(C₂-4 alkylene)CN, -heterocyclyl optionally substituted with 1-4 R⁶, —CH₂C(═O)heterocyclyl, and -carbocyclyl optionally substituted with 4 R⁷, wherein the —(C₁₋₂ alkylene) or (C₂₋₄ alkylene) is optionally substituted with 1-2 halide.

9. The compound of claim 7, wherein R² is selected from the group consisting of —(C₂-4 alkylene)CN, -heterocyclyl optionally substituted with 1-4 R⁶, —CH₂C(═O)heterocyclyl, and -carbocyclyl optionally substituted with 4 R⁷, wherein the —(C₁₋₂ alkylene) or (C₂₋₄ alkylene) is optionally substituted with 1-2 halide.

10. The compound of claim 8, wherein R³ is methyl.

11. The compound of claim 9, wherein R³ is methyl.

12. The compound of claim 1, wherein R⁴ is selected from the group consisting of H and F.

13. The compound of claim 10, wherein each R⁶ is independently selected from the group consisting of F, unsubstituted —(C₁₋₃ alkyl), unsubstituted -heterocyclyl, —CH₂CH₂OH, —CH₂CH₂OMe, —SO₂Me, and —(CH₂)ₚC(═O)R¹⁰.

14. The compound of claim 11, wherein each R⁶ is independently selected from the group consisting of F, unsubstituted —(C₁₋₃ alkyl), unsubstituted -heterocyclyl, —CH₂CH₂OH, —CH₂CH₂OMe, —SO₂Me, and —(CH₂)ₚC(═O)R¹⁰.

15. The compound of claim 13, wherein each R is independently selected from the group consisting of F, unsubstituted —(C₁₋₃ alkyl), —OH, and —NHC(═O)R¹¹.

16. The compound of claim 14, wherein each R is independently selected from the group consisting of F, unsubstituted —(C₁₋₃ alkyl), —OH, and —NHC(═O)R¹¹.

17. The compound of claim 1, wherein the compound of Formula I is selected from the group consisting of:
(R)-1-(3,3-difluoro-4-((4-methoxy-5-(pyrazolo[1,5-a]pyridin-5-yl)pyrrolo[2,1-f][1,2,4]triazin-2-yl)amino)piperidin-1-yl)ethan-1-one [1];
(R)—N-(3,3-difluoro-1-(oxetan-3-yl)piperidin-4-yl)-4-methoxy-5-(pyrazolo[1,5-a]pyridin-5-yl)pyrrolo[2,1-f][1,2,4]triazin-2-amine [2];
5-(2-((2-cyano-2-methylpropyl)amino)-4-methoxypyrrolo[2,1-f][1,2,4]triazin-5-yl)-N-methylpyrazolo[1,5-a]pyridine-3-carboxamide [3];
5-(2-((4,4-difluorocyclohexyl)amino)-4-methoxypyrrolo[2,1-f][1,2,4]triazin-5-yl)-N-methylpyrazolo[1,5-a]pyridine-3-carboxamide [4];
5-(2-(((1r,4r)-4-hydroxy-4-methylcyclohexyl)amino)-4-methoxypyrrolo[2,1-f][1,2,4]triazin-5-yl)-N-(methyl-d₃)pyrazolo[1,5-a]pyridine-3-carboxamide [5];
N-ethyl-5-(2-(((1r,4r)-4-hydroxy-4-methylcyclohexyl)amino)-4-methoxypyrrolo[2,1-f][1,2,4]triazin-5-yl)pyrazolo[1,5-a]pyridine-3-carboxamide [6];
N-(2,2-difluoroethyl)-5-(2-(((1r,4r)-4-hydroxy-4-methylcyclohexyl)amino)-4-methoxypyrrolo[2,1-f][1,2,4]triazin-5-yl)pyrazolo[1,5-a]pyridine-3-carboxamide [7];
5-(2-(((1r,4r)-4-hydroxy-4-methylcyclohexyl)amino)-4-methoxypyrrolo[2,1-f][1,2,4]triazin-5-yl)-N-isopropylpyrazolo[1,5-a]pyridine-3-carboxamide [8];
5-(2-(((3R,4S)-3-fluoro-1-(oxetan-3-yl)piperidin-4-yl)amino)-4-methoxypyrrolo[2,1-f][1,2,4]triazin-5-yl)-N-methylpyrazolo[1,5-a]pyridine-3-carboxamide [9];
(R)-5-(2-((3,3-difluoro-1-methylpiperidin-4-yl)amino)-4-methoxypyrrolo[2,1-f][1,2,4]triazin-5-yl)-N-methylpyrazolo[1,5-a]pyridine-3-carboxamide [10];
(R)-5-(2-((1-acetyl-3,3-difluoropiperidin-4-yl)amino)-4-methoxypyrrolo[2,1-f][1,2,4]triazin-5-yl)-N-methylpyrazolo[1,5-a]pyridine-3-carboxamide [11];
(R)-5-(2-((3,3-difluoro-1-(oxetan-3-yl)piperidin-4-yl)amino)-4-methoxypyrrolo[2,1-f][1,2,4]triazin-5-yl)-N-methylpyrazolo[1,5-a]pyridine-3-carboxamide [12];
5-(2-((2-cyano-2-methylpropyl)amino)-6-fluoro-4-methoxypyrrolo[2,1-f][1,2,4]triazin-5-yl)-N-methylpyrazolo[1,5-a]pyridine-3-carboxamide [13];
5-(2-((4,4-difluorocyclohexyl)amino)-6-fluoro-4-methoxypyrrolo[2,1-f][1,2,4]triazin-5-yl)-N-methylpyrazolo[1,5-a]pyridine-3-carboxamide [14];
(R)-5-(2-((4,4-difluoro-1-methylpyrrolidin-3-yl)amino)-6-fluoro-4-methoxypyrrolo[2,1-f][1,2,4]triazin-5-yl)-N-methylpyrazolo[1,5-a]pyridine-3-carboxamide [15];
(R)-5-(2-((4,4-difluoro-1-(oxetan-3-yl)pyrrolidin-3-yl)amino)-6-fluoro-4-methoxypyrrolo[2,1-f][1,2,4]triazin-5-yl)-N-methylpyrazolo[1,5-a]pyridine-3-carboxamide [16];
5-(6-fluoro-4-methoxy-2-((1-(oxetan-3-yl)piperidin-4-yl)amino)pyrrolo[2,1-f][1,2,4]triazin-5-yl)-N-methylpyrazolo[1,5-a]pyridine-3-carboxamide [17];
5-(6-fluoro-2-(((3S,4R)-3-fluoro-1-(oxetan-3-yl)piperidin-4-yl)amino)-4-methoxypyrrolo[2,1-f][1,2,4]triazin-5-yl)-N-methylpyrazolo[1,5-a]pyridine-3-carboxamide [18];
5-(6-fluoro-2-(((3R,4R)-3-fluoro-1-(oxetan-3-yl)piperidin-4-yl)amino)-4-methoxypyrrolo[2,1-f][1,2,4]triazin-5-yl)-N-methylpyrazolo[1,5-a]pyridine-3-carboxamide [19];
5-(6-fluoro-2-(((3S,4S)-3-fluoro-1-(oxetan-3-yl)piperidin-4-yl)amino)-4-methoxypyrrolo[2,1-f][1,2,4]triazin-5-yl)-N-methylpyrazolo[1,5-a]pyridine-3-carboxamide [20];
5-(6-fluoro-2-(((3R,4S)-3-fluoro-1-(oxetan-3-yl)piperidin-4-yl)amino)-4-methoxypyrrolo[2,1-f][1,2,4]triazin-5-yl)-N-methylpyrazolo[1,5-a]pyridine-3-carboxamide [21];
(R)-5-(2-((3,3-difluoro-1-methylpiperidin-4-yl)amino)-6-fluoro-4-methoxypyrrolo[2,1-f][1,2,4]triazin-5-yl)-N-methylpyrazolo[1,5-a]pyridine-3-carboxamide [22];
(R)-5-(2-((3,3-difluoro-1-(methyl-d₃)piperidin-4-yl)amino)-6-fluoro-4-methoxypyrrolo[2,1-f][1,2,4]triazin-5-yl)-N-methylpyrazolo[1,5-a]pyridine-3-carboxamide [23];
(R)-5-(2-((3,3-difluoro-1-(oxetan-3-yl)piperidin-4-yl)amino)-6-fluoro-4-methoxypyrrolo[2,1-f][1,2,4]triazin-5-yl)-N-methylpyrazolo[1,5-a]pyridine-3-carboxamide [24];
5-(2-((2-oxaspiro[3.5]nonan-7-yl)amino)-6-fluoro-4-methoxypyrrolo[2,1-f][1,2,4]triazin-5-yl)-N-methylpyrazolo[1,5-a]pyridine-3-carboxamide [25];
5-(2-((2-cyano-2-methylpropyl)amino)-6-fluoro-4-methoxypyrrolo[2,1-f][1,2,4]triazin-5-yl)-N-isopropylpyrazolo[1,5-a]pyridine-3-carboxamide [26];
5-(2-((4,4-difluorocyclohexyl)amino)-6-fluoro-4-methoxypyrrolo[2,1-f][1,2,4]triazin-5-yl)-N-isopropylpyrazolo[1,5-a]pyridine-3-carboxamide [27];
5-(6-fluoro-2-(((1s,4s)-4-hydroxy-4-methylcyclohexyl)amino)-4-methoxypyrrolo[2,1-f][1,2,4]triazin-5-yl)-N-isopropylpyrazolo[1,5-a]pyridine-3-carboxamide [28];
5-(6-fluoro-2-(((1r,4r)-4-hydroxy-4-methylcyclohexyl)amino)-4-methoxypyrrolo[2,1-f][1,2,4]triazin-5-yl)-N-isopropylpyrazolo[1,5-a]pyridine-3-carboxamide [29];
(R)-5-(2-((4,4-difluoro-1-methylpyrrolidin-3-yl)amino)-6-fluoro-4-methoxypyrrolo[2,1-f][1,2,4]triazin-5-yl)-N-isopropylpyrazolo[1,5-a]pyridine-3-carboxamide [30];
(R)-5-(2-((4,4-difluoro-1-(oxetan-3-yl)pyrrolidin-3-yl)amino)-6-fluoro-4-methoxypyrrolo[2,1-f][1,2,4]triazin-5-yl)-N-isopropylpyrazolo[1,5-a]pyridine-3-carboxamide [31];
5-(6-fluoro-4-methoxy-2-((1-(oxetan-3-yl)piperidin-4-yl)amino)pyrrolo[2,1-f][1,2,4]triazin-5-yl)-N-isopropylpyrazolo[1,5-a]pyridine-3-carboxamide [32];
5-(6-fluoro-2-(((3S,4R)-3-fluoro-1-(oxetan-3-yl)piperidin-4-yl)amino)-4-methoxypyrrolo[2,1-f][1,2,4]triazin-5-yl)-N-isopropylpyrazolo[1,5-a]pyridine-3-carboxamide [33];
5-(6-fluoro-2-(((3R,4R)-3-fluoro-1-(oxetan-3-yl)piperidin-4-yl)amino)-4-methoxypyrrolo[2,1-f][1,2,4]triazin-5-yl)-N-isopropylpyrazolo[1,5-a]pyridine-3-carboxamide [34];
5-(6-fluoro-2-(((3S,4S)-3-fluoro-1-(oxetan-3-yl)piperidin-4-yl)amino)-4-methoxypyrrolo[2,1-f][1,2,4]triazin-5-yl)-N-isopropylpyrazolo[1,5-a]pyridine-3-carboxamide [35];
5-(6-fluoro-2-(((3R,4S)-3-fluoro-1-(oxetan-3-yl)piperidin-4-yl)amino)-4-methoxypyrrolo[2,1-f][1,2,4]triazin-5-yl)-N-isopropylpyrazolo[1,5-a]pyridine-3-carboxamide [36];
(R)-5-(2-((3,3-difluoro-1-methylpiperidin-4-yl)amino)-6-fluoro-4-methoxypyrrolo[2,1-f][1,2,4]triazin-5-yl)-N-isopropylpyrazolo[1,5-a]pyridine-3-carboxamide [37];
(R)-5-(2-((3,3-difluoro-1-(methyl-d3)piperidin-4-yl)amino)-6-fluoro-4-methoxypyrrolo[2,1-f][1,2,4]triazin-5-yl)-N-isopropylpyrazolo[1,5-a]pyridine-3-carboxamide [38];
(R)-5-(2-((3,3-difluoro-1-(oxetan-3-yl)piperidin-4-yl)amino)-6-fluoro-4-methoxypyrrolo[2,1-f][1,2,4]triazin-5-yl)-N-isopropylpyrazolo[1,5-a]pyridine-3-carboxamide [39];
5-(2-((2-oxaspiro[3.5]nonan-7-yl)amino)-6-fluoro-4-methoxypyrrolo[2,1-f][1,2,4]triazin-5-yl)-N-isopropylpyrazolo[1,5-a]pyridine-3-carboxamide [40];
6-(2-((2-cyano-2-methylpropyl)amino)-4-methoxypyrrolo[2,1-f][1,2,4]triazin-5-yl)-N-methylimidazo[1,2-a]pyridine-3-carboxamide [41];
6-(2-(((3R,4S)-3-fluoro-1-(oxetan-3-yl)piperidin-4-yl)amino)-4-methoxypyrrolo[2,1-f][1,2,4]triazin-5-yl)-N-methylimidazo[1,2-a]pyridine-3-carboxamide [42];
(R)-6-(2-((3,3-difluoro-1-methylpiperidin-4-yl)amino)-4-methoxypyrrolo[2,1-f][1,2,4]triazin-5-yl)-N-methylimidazo[1,2-a]pyridine-3-carboxamide [43];
(R)-6-(2-((3,3-difluoro-1-(oxetan-3-yl)piperidin-4-yl)amino)-4-methoxypyrrolo[2,1-f][1,2,4]triazin-5-yl)-N-methylimidazo[1,2-a]pyridine-3-carboxamide [44];
6-(2-((2-cyano-2-methylpropyl)amino)-6-fluoro-4-methoxypyrrolo[2,1-f][1,2,4]triazin-5-yl)-N-methylimidazo[1,2-a]pyridine-3-carboxamide [45];
6-(2-((4,4-difluorocyclohexyl)amino)-6-fluoro-4-methoxypyrrolo[2,1-f][1,2,4]triazin-5-yl)-N-methylimidazo[1,2-a]pyridine-3-carboxamide [46];
(R)-6-(2-((4,4-difluoro-1-methylpyrrolidin-3-yl)amino)-6-fluoro-4-methoxypyrrolo[2,1-f][1,2,4]triazin-5-yl)-N-methylimidazo[1,2-a]pyridine-3-carboxamide [47];
(R)-6-(2-((4,4-difluoro-1-(oxetan-3-yl)pyrrolidin-3-yl)amino)-6-fluoro-4-methoxypyrrolo[2,1-f][1,2,4]triazin-5-yl)-N-methylimidazo[1,2-a]pyridine-3-carboxamide [48];
6-(6-fluoro-4-methoxy-2-((1-(oxetan-3-yl)piperidin-4-yl)amino)pyrrolo[2,1-f][1,2,4]triazin-5-yl)-N-methylimidazo[1,2-a]pyridine-3-carboxamide [49];
6-(6-fluoro-2-(((3S,4R)-3-fluoro-1-(oxetan-3-yl)piperidin-4-yl)amino)-4-methoxypyrrolo[2,1-f][1,2,4]triazin-5-yl)-N-methylimidazo[1,2-a]pyridine-3-carboxamide [50];
6-(6-fluoro-2-(((3R,4R)-3-fluoro-1-(oxetan-3-yl)piperidin-4-yl)amino)-4-methoxypyrrolo[2,1-f][1,2,4]triazin-5-yl)-N-methylimidazo[1,2-a]pyridine-3-carboxamide [51];
6-(6-fluoro-2-(((3S,4S)-3-fluoro-1-(oxetan-3-yl)piperidin-4-yl)amino)-4-methoxypyrrolo[2,1-f][1,2,4]triazin-5-yl)-N-methylimidazo[1,2-a]pyridine-3-carboxamide [52];
6-(6-fluoro-2-(((3R,4S)-3-fluoro-1-(oxetan-3-yl)piperidin-4-yl)amino)-4-methoxypyrrolo[2,1-f][1,2,4]triazin-5-yl)-N-methylimidazo[1,2-a]pyridine-3-carboxamide [53];
(R)-6-(2-((3,3-difluoro-1-methylpiperidin-4-yl)amino)-6-fluoro-4-methoxypyrrolo[2,1-f][1,2,4]triazin-5-yl)-N-methylimidazo[1,2-a]pyridine-3-carboxamide [54];
(R)-6-(2-((3,3-difluoro-1-(methyl-d₃)piperidin-4-yl)amino)-6-fluoro-4-methoxypyrrolo[2,1-f][1,2,4]triazin-5-yl)-N-methylimidazo[1,2-a]pyridine-3-carboxamide [55];
(R)-6-(2-((3,3-difluoro-1-(oxetan-3-yl)piperidin-4-yl)amino)-6-fluoro-4-methoxypyrrolo[2,1-f][1,2,4]triazin-5-yl)-N-methylimidazo[1,2-a]pyridine-3-carboxamide [56];
(1r,4r)-4-((5-(3-(difluoromethyl)-8-fluoroimidazo[1,2-a]pyridin-6-yl)-4-methoxypyrrolo[2,1-f][1,2,4]triazin-2-yl)amino)-1-methylcyclohexan-1-ol [57];
(R)-1-(4-((5-(3-(difluoromethyl)-8-fluoroimidazo[1,2-a]pyridin-6-yl)-4-methoxypyrrolo[2,1-f][1,2,4]triazin-2-yl)amino)-3,3-difluoropiperidin-1-yl)ethan-1-one [58];
3-((5-(3-(difluoromethyl)-8-fluoroimidazo[1,2-a]pyridin-6-yl)-6-fluoro-4-methoxypyrrolo[2,1-f][1,2,4]triazin-2-yl)amino)-2,2-dimethylpropanenitrile [59];
N-(4,4-difluorocyclohexyl)-5-(3-(difluoromethyl)-8-fluoroimidazo[1,2-a]pyridin-6-yl)-6-fluoro-4-methoxypyrrolo[2,1-f][1,2,4]triazin-2-amine [60];
(1s,4s)-4-((5-(3-(difluoromethyl)-8-fluoroimidazo[1,2-a]pyridin-6-yl)-6-fluoro-4-methoxypyrrolo[2,1-f][1,2,4]triazin-2-yl)amino)-1-methylcyclohexan-1-ol [61];
(1r,4r)-4-((5-(3-(difluoromethyl)-8-fluoroimidazo[1,2-a]pyridin-6-yl)-6-fluoro-4-methoxypyrrolo[2,1-f][1,2,4]triazin-2-yl)amino)-1-methylcyclohexan-1-ol [62];
(R)—N-(4,4-difluoro-1-methylpyrrolidin-3-yl)-5-(3-(difluoromethyl)-8-fluoroimidazo[1,2-a]pyridin-6-yl)-6-fluoro-4-methoxypyrrolo[2,1-f][1,2,4]triazin-2-amine [63];
(R)—N-(4,4-difluoro-1-(oxetan-3-yl)pyrrolidin-3-yl)-5-(3-(difluoromethyl)-8-fluoroimidazo[1,2-a]pyridin-6-yl)-6-fluoro-4-methoxypyrrolo[2,1-f][1,2,4]triazin-2-amine [64];
5-(3-(difluoromethyl)-8-fluoroimidazo[1,2-a]pyridin-6-yl)-6-fluoro-4-methoxy-N-(1-(oxetan-3-yl)piperidin-4-yl)pyrrolo[2,1-f][1,2,4]triazin-2-amine [65];
5-(3-(difluoromethyl)-8-fluoroimidazo[1,2-a]pyridin-6-yl)-6-fluoro-N-((3S,4R)-3-fluoro-1-(oxetan-3-yl)piperidin-4-yl)-4-methoxypyrrolo[2,1-f][1,2,4]triazin-2-amine [66];
5-(3-(difluoromethyl)-8-fluoroimidazo[1,2-a]pyridin-6-yl)-6-fluoro-N-((3R,4R)-3-fluoro-1-(oxetan-3-yl)piperidin-4-yl)-4-methoxypyrrolo[2,1-f][1,2,4]triazin-2-amine [67];
5-(3-(difluoromethyl)-8-fluoroimidazo[1,2-a]pyridin-6-yl)-6-fluoro-N-((3S,4S)-3-fluoro-1-(oxetan-3-yl)piperidin-4-yl)-4-methoxypyrrolo[2,1-f][1,2,4]triazin-2-amine [68];
5-(3-(difluoromethyl)-8-fluoroimidazo[1,2-a]pyridin-6-yl)-6-fluoro-N-((3R,4S)-3-fluoro-1-(oxetan-3-yl)piperidin-4-yl)-4-methoxypyrrolo[2,1-f][1,2,4]triazin-2-amine [69];
(R)—N-(3,3-difluoro-1-methylpiperidin-4-yl)-5-(3-(difluoromethyl)-8-fluoroimidazo[1,2-a]pyridin-6-yl)-6-fluoro-4-methoxypyrrolo[2,1-f][1,2,4]triazin-2-amine [70];
(R)—N-(3,3-difluoro-1-(methyl-d₃)piperidin-4-yl)-5-(3-(difluoromethyl)-8-fluoroimidazo[1,2-a]pyridin-6-yl)-6-fluoro-4-methoxypyrrolo[2,1-f][1,2,4]triazin-2-amine [71];
(R)—N-(3,3-difluoro-1-(oxetan-3-yl)piperidin-4-yl)-5-(3-(difluoromethyl)-8-fluoroimidazo[1,2-a]pyridin-6-yl)-6-fluoro-4-methoxypyrrolo[2,1-f][1,2,4]triazin-2-amine [72];
5-(3-(difluoromethyl)-8-fluoroimidazo[1,2-a]pyridin-6-yl)-6-fluoro-4-methoxy-N-(2-oxaspiro[3.5]nonan-7-yl)pyrrolo[2,1-f][1,2,4]triazin-2-amine [73];
6-(2-((4,4-difluorocyclohexyl)amino)-4-methoxypyrrolo[2,1-f][1,2,4]triazin-5-yl)-8-fluoro-N-methylimidazo[1,2-a]pyridine-3-carboxamide [74];
8-fluoro-6-(2-(((3R,4S)-3-fluoro-1-(oxetan-3-yl)piperidin-4-yl)amino)-4-methoxypyrrolo[2,1-f][1,2,4]triazin-5-yl)-N-methylimidazo[1,2-a]pyridine-3-carboxamide [75];
(R)-6-(2-((3,3-difluoro-1-methylpiperidin-4-yl)amino)-4-methoxypyrrolo[2,1-f][1,2,4]triazin-5-yl)-8-fluoro-N-methylimidazo[1,2-a]pyridine-3-carboxamide [76];
(R)-6-(2-((3,3-difluoro-1-(oxetan-3-yl)piperidin-4-yl)amino)-4-methoxypyrrolo[2,1-f][1,2,4]triazin-5-yl)-8-fluoro-N-methylimidazo[1,2-a]pyridine-3-carboxamide [77];
6-(2-((2-cyano-2-methylpropyl)amino)-6-fluoro-4-methoxypyrrolo[2,1-f][1,2,4]triazin-5-yl)-8-fluoro-N-methylimidazo[1,2-a]pyridine-3-carboxamide [78];
6-(2-((4,4-difluorocyclohexyl)amino)-6-fluoro-4-methoxypyrrolo[2,1-f][1,2,4]triazin-5-yl)-8-fluoro-N-methylimidazo[1,2-a]pyridine-3-carboxamide [79];
(R)-6-(2-((4,4-difluoro-1-methylpyrrolidin-3-yl)amino)-6-fluoro-4-methoxypyrrolo[2,1-f][1,2,4]triazin-5-yl)-8-fluoro-N-methylimidazo[1,2-a]pyridine-3-carboxamide [80];
(R)-6-(2-((4,4-difluoro-1-(oxetan-3-yl)pyrrolidin-3-yl)amino)-6-fluoro-4-methoxypyrrolo[2,1-f][1,2,4]triazin-5-yl)-8-fluoro-N-methylimidazo[1,2-a]pyridine-3-carboxamide [81];
8-fluoro-6-(6-fluoro-4-methoxy-2-((1-(oxetan-3-yl)piperidin-4-yl)amino)pyrrolo[2,1-f][1,2,4]triazin-5-yl)-N-methylimidazo[1,2-a]pyridine-3-carboxamide [82];
8-fluoro-6-(6-fluoro-2-(((3S,4R)-3-fluoro-1-(oxetan-3-yl)piperidin-4-yl)amino)-4-methoxypyrrolo[2,1-f][1,2,4]triazin-5-yl)-N-methylimidazo[1,2-a]pyridine-3-carboxamide [83];
8-fluoro-6-(6-fluoro-2-(((3R,4R)-3-fluoro-1-(oxetan-3-yl)piperidin-4-yl)amino)-4-methoxypyrrolo[2,1-f][1,2,4]triazin-5-yl)-N-methylimidazo[1,2-a]pyridine-3-carboxamide [84];
8-fluoro-6-(6-fluoro-2-(((3S,4S)-3-fluoro-1-(oxetan-3-yl)piperidin-4-yl)amino)-4-methoxypyrrolo[2,1-f][1,2,4]triazin-5-yl)-N-methylimidazo[1,2-a]pyridine-3-carboxamide [85];
8-fluoro-6-(6-fluoro-2-(((3R,4S)-3-fluoro-1-(oxetan-3-yl)piperidin-4-yl)amino)-4-methoxypyrrolo[2,1-f][1,2,4]triazin-5-yl)-N-methylimidazo[1,2-a]pyridine-3-carboxamide [86];
(R)-6-(2-((3,3-difluoro-1-methylpiperidin-4-yl)amino)-6-fluoro-4-methoxypyrrolo[2,1-f][1,2,4]triazin-5-yl)-8-fluoro-N-methylimidazo[1,2-a]pyridine-3-carboxamide [87];
(R)-6-(2-((3,3-difluoro-1-(methyl-d₃)piperidin-4-yl)amino)-6-fluoro-4-methoxypyrrolo[2,1-f][1,2,4]triazin-5-yl)-8-fluoro-N-methylimidazo[1,2-a]pyridine-3-carboxamide [88];
(R)-6-(2-((3,3-difluoro-1-(oxetan-3-yl)piperidin-4-yl)amino)-6-fluoro-4-methoxypyrrolo[2,1-f][1,2,4]triazin-5-yl)-8-fluoro-N-methylimidazo[1,2-a]pyridine-3-carboxamide [89];
6-(2-((2-oxaspiro[3.5]nonan-7-yl)amino)-6-fluoro-4-methoxypyrrolo[2,1-f][1,2,4]triazin-5-yl)-8-fluoro-N-methylimidazo[1,2-a]pyridine-3-carboxamide [90];
3-((5-(1-(2-fluoroethyl)-1H-benzo[d]imidazol-6-yl)-4-methoxypyrrolo[2,1-f][1,2,4]triazin-2-yl)amino)-2,2-dimethylpropanenitrile [91];
(1s,4s)-4-((5-(1-(2-fluoroethyl)-1H-benzo[d]imidazol-6-yl)-4-methoxypyrrolo[2,1-f][1,2,4]triazin-2-yl)amino)-1-methylcyclohexan-1-ol [92];
(1r,4r)-4-((5-(1-(2-fluoroethyl)-1H-benzo[d]imidazol-6-yl)-4-methoxypyrrolo[2,1-f][1,2,4]triazin-2-yl)amino)-1-methylcyclohexan-1-ol [93];
(R)—N-(4,4-difluoro-1-methylpyrrolidin-3-yl)-5-(1-(2-fluoroethyl)-1H-benzo[d]imidazol-6-yl)-4-methoxypyrrolo[2,1-f][1,2,4]triazin-2-amine [94];
(R)-1-(3,3-difluoro-4-((5-(1-(2-fluoroethyl)-1H-benzo[d]imidazol-6-yl)-4-methoxypyrrolo[2,1-f][1,2,4]triazin-2-yl)amino)pyrrolidin-1-yl)ethan-1-one [95];
(R)—N-(4,4-difluoro-1-(oxetan-3-yl)pyrrolidin-3-yl)-5-(1-(2-fluoroethyl)-1H-benzo[d]imidazol-6-yl)-4-methoxypyrrolo[2,1-f][1,2,4]triazin-2-amine [96];
5-(1-(2-fluoroethyl)-1H-benzo[d]imidazol-6-yl)-4-methoxy-N-(1-(oxetan-3-yl)piperidin-4-yl)pyrrolo[2,1-f][1,2,4]triazin-2-amine [97];
N-((3S,4R)-3-fluoro-1-methylpiperidin-4-yl)-5-(1-(2-fluoroethyl)-1H-benzo[d]imidazol-6-yl)-4-methoxypyrrolo[2,1-f][1,2,4]triazin-2-amine [98];
N-((3R,4S)-3-fluoro-1-methylpiperidin-4-yl)-5-(1-(2-fluoroethyl)-1H-benzo[d]imidazol-6-yl)-4-methoxypyrrolo[2,1-f][1,2,4]triazin-2-amine [99]; and
1-((3R,4S)-3-fluoro-4-((5-(1-(2-fluoroethyl)-1H-benzo[d]imidazol-6-yl)-4-methoxypyrrolo[2,1-f][1,2,4]triazin-2-yl)amino)piperidin-1-yl)ethan-1-one [100]; or a pharmaceutically acceptable salt thereof.

18. The compound of claim 1, wherein the compound of Formula I is selected from the group consisting of
1-((3R,4S)-3-fluoro-4-((5-(1-(2-fluoroethyl)-1H-benzo[d]imidazol-6-yl)-4-methoxypyrrolo[2,1-f][1,2,4]triazin-2-yl)amino)piperidin-1-yl)ethan-1-one-2,2,2-d₃ [101];
1-((3R,4S)-3-fluoro-4-((5-(1-(2-fluoroethyl)-1H-benzo[d]imidazol-6-yl)-4-methoxypyrrolo[2,1-f][1,2,4]triazin-2-yl)amino)piperidin-1-yl)-2-hydroxyethan-1-one [102];
N-((3S,4R)-3-fluoro-1-(oxetan-3-yl)piperidin-4-yl)-5-(1-(2-fluoroethyl)-1H-benzo[d]imidazol-6-yl)-4-methoxypyrrolo[2,1-f][1,2,4]triazin-2-amine [103];
N-((3R,4R)-3-fluoro-1-(oxetan-3-yl)piperidin-4-yl)-5-(1-(2-fluoroethyl)-1H-benzo[d]imidazol-6-yl)-4-methoxypyrrolo[2,1-f][1,2,4]triazin-2-amine [104];
N-((3S,4S)-3-fluoro-1-(oxetan-3-yl)piperidin-4-yl)-5-(1-(2-fluoroethyl)-1H-benzo[d]imidazol-6-yl)-4-methoxypyrrolo[2,1-f][1,2,4]triazin-2-amine [105];
N-((3R,4S)-3-fluoro-1-(oxetan-3-yl)piperidin-4-yl)-5-(1-(2-fluoroethyl)-1H-benzo[d]imidazol-6-yl)-4-methoxypyrrolo[2,1-f][1,2,4]triazin-2-amine [106];
(R)—N-(3,3-difluoro-1-methylpiperidin-4-yl)-5-(1-(2-fluoroethyl)-1H-benzo[d]imidazol-6-yl)-4-methoxypyrrolo[2,1-f][1,2,4]triazin-2-amine [107];
(R)—N-(3,3-difluoro-1-(methyl-d₃)piperidin-4-yl)-5-(1-(2-fluoroethyl)-1H-benzo[d]imidazol-6-yl)-4-methoxypyrrolo[2,1-f][1,2,4]triazin-2-amine [108];
(R)-1-(3,3-difluoro-4-((5-(1-(2-fluoroethyl)-1H-benzo[d]imidazol-6-yl)-4-methoxypyrrolo[2,1-f][1,2,4]triazin-2-yl)amino)piperidin-1-yl)ethan-1-one [109];
(R)-1-(3,3-difluoro-4-((5-(1-(2-fluoroethyl)-1H-benzo[d]imidazol-6-yl)-4-methoxypyrrolo[2,1-f][1,2,4]triazin-2-yl)amino)piperidin-1-yl)ethan-1-one-2,2,2-d₃ [110];
(R)-1-(3,3-difluoro-4-((5-(1-(2-fluoroethyl)-1H-benzo[d]imidazol-6-yl)-4-methoxypyrrolo[2,1-f][1,2,4]triazin-2-yl)amino)piperidin-1-yl)-2-hydroxyethan-1-one [111];
(R)—N-(3,3-difluoro-1-(methylsulfonyl)piperidin-4-yl)-5-(1-(2-fluoroethyl)-1H-benzo[d]imidazol-6-yl)-4-methoxypyrrolo[2,1-f][1,2,4]triazin-2-amine [112];
(R)—N-(3,3-difluoro-1-(oxetan-3-yl)piperidin-4-yl)-5-(1-(2-fluoroethyl)-1H-benzo[d]imidazol-6-yl)-4-methoxypyrrolo[2,1-f][1,2,4]triazin-2-amine [113];
5-(1-(2-fluoroethyl)-1H-benzo[d]imidazol-6-yl)-4-methoxy-N-(2-oxaspiro[3.5]nonan-7-yl)pyrrolo[2,1-f][1,2,4]triazin-2-amine [114];
3-((6-fluoro-5-(1-(2-fluoroethyl)-1H-benzo[d]imidazol-6-yl)-4-methoxypyrrolo[2,1-f][1,2,4]triazin-2-yl)amino)-2,2-dimethylpropanenitrile [115];
(1s,4s)-4-((6-fluoro-5-(1-(2-fluoroethyl)-1H-benzo[d]imidazol-6-yl)-4-methoxypyrrolo[2,1-f][1,2,4]triazin-2-yl)amino)-1-methylcyclohexan-1-ol [116];
(1r,4r)-4-((6-fluoro-5-(1-(2-fluoroethyl)-1H-benzo[d]imidazol-6-yl)-4-methoxypyrrolo[2,1-f][1,2,4]triazin-2-yl)amino)-1-methylcyclohexan-1-ol [117];
(R)—N-(4,4-difluoro-1-methylpyrrolidin-3-yl)-6-fluoro-5-(1-(2-fluoroethyl)-1H-benzo[d]imidazol-6-yl)-4-methoxypyrrolo[2,1-f][1,2,4]triazin-2-amine [118];
(R)-1-(3,3-difluoro-4-((6-fluoro-5-(1-(2-fluoroethyl)-1H-benzo[d]imidazol-6-yl)-4-methoxypyrrolo[2,1-f][1,2,4]triazin-2-yl)amino)pyrrolidin-1-yl)ethan-1-one [119];
(R)—N-(4,4-difluoro-1-(oxetan-3-yl)pyrrolidin-3-yl)-6-fluoro-5-(1-(2-fluoroethyl)-1H-benzo[d]imidazol-6-yl)-4-methoxypyrrolo[2,1-f][1,2,4]triazin-2-amine [120];
6-fluoro-5-(1-(2-fluoroethyl)-1H-benzo[d]imidazol-6-yl)-4-methoxy-N-(1-(oxetan-3-yl)piperidin-4-yl)pyrrolo[2,1-f][1,2,4]triazin-2-amine [121];
6-fluoro-N-((3S,4R)-3-fluoro-1-methylpiperidin-4-yl)-5-(1-(2-fluoroethyl)-1H-benzo[d]imidazol-6-yl)-4-methoxypyrrolo[2,1-f][1,2,4]triazin-2-amine [122];
6-fluoro-N-((3R,4S)-3-fluoro-1-methylpiperidin-4-yl)-5-(1-(2-fluoroethyl)-1H-benzo[d]imidazol-6-yl)-4-methoxypyrrolo[2,1-f][1,2,4]triazin-2-amine [123];
1-((3R,4S)-3-fluoro-4-((6-fluoro-5-(1-(2-fluoroethyl)-1H-benzo[d]imidazol-6-yl)-4-methoxypyrrolo[2,1-f][1,2,4]triazin-2-yl)amino)piperidin-1-yl)ethan-1-one [124];
1-((3R,4S)-3-fluoro-4-((6-fluoro-5-(1-(2-fluoroethyl)-1H-benzo[d]imidazol-6-yl)-4-methoxypyrrolo[2,1-f][1,2,4]triazin-2-yl)amino)piperidin-1-yl)ethan-1-one-2,2,2-d₃ [125];
1-((3R,4S)-3-fluoro-4-((6-fluoro-5-(1-(2-fluoroethyl)-1H-benzo[d]imidazol-6-yl)-4-methoxypyrrolo[2,1-f][1,2,4]triazin-2-yl)amino)piperidin-1-yl)-2-hydroxyethan-1-one [126];
6-fluoro-N-((3S,4R)-3-fluoro-1-(oxetan-3-yl)piperidin-4-yl)-5-(1-(2-fluoroethyl)-1H-benzo[d]imidazol-6-yl)-4-methoxypyrrolo[2,1-f][1,2,4]triazin-2-amine [127];
6-fluoro-N-((3R,4R)-3-fluoro-1-(oxetan-3-yl)piperidin-4-yl)-5-(1-(2-fluoroethyl)-1H-benzo[d]imidazol-6-yl)-4-methoxypyrrolo[2,1-f][1,2,4]triazin-2-amine [128];
6-fluoro-N-((3S,4S)-3-fluoro-1-(oxetan-3-yl)piperidin-4-yl)-5-(1-(2-fluoroethyl)-1H-benzo[d]imidazol-6-yl)-4-methoxypyrrolo[2,1-f][1,2,4]triazin-2-amine [129];
6-fluoro-N-((3R,4S)-3-fluoro-1-(oxetan-3-yl)piperidin-4-yl)-5-(1-(2-fluoroethyl)-1H-benzo[d]imidazol-6-yl)-4-methoxypyrrolo[2,1-f][1,2,4]triazin-2-amine [130];
(R)—N-(3,3-difluoro-1-methylpiperidin-4-yl)-6-fluoro-5-(1-(2-fluoroethyl)-1H-benzo[d]imidazol-6-yl)-4-methoxypyrrolo[2,1-f][1,2,4]triazin-2-amine [131];
(R)—N-(3,3-difluoro-1-(methyl-d₃)piperidin-4-yl)-6-fluoro-5-(1-(2-fluoroethyl)-1H-benzo[d]imidazol-6-yl)-4-methoxypyrrolo[2,1-f][1,2,4]triazin-2-amine [132];
(R)-1-(3,3-difluoro-4-((6-fluoro-5-(1-(2-fluoroethyl)-1H-benzo[d]imidazol-6-yl)-4-methoxypyrrolo[2,1-f][1,2,4]triazin-2-yl)amino)piperidin-1-yl)ethan-1-one [133];
(R)-1-(3,3-difluoro-4-((6-fluoro-5-(1-(2-fluoroethyl)-1H-benzo[d]imidazol-6-yl)-4-methoxypyrrolo[2,1-f][1,2,4]triazin-2-yl)amino)piperidin-1-yl)ethan-1-one-2,2,2-d₃ [134];
(R)-1-(3,3-difluoro-4-((6-fluoro-5-(1-(2-fluoroethyl)-1H-benzo[d]imidazol-6-yl)-4-methoxypyrrolo[2,1-f][1,2,4]triazin-2-yl)amino)piperidin-1-yl)-2-hydroxyethan-1-one [135];
(R)—N-(3,3-difluoro-1-(methylsulfonyl)piperidin-4-yl)-6-fluoro-5-(1-(2-fluoroethyl)-1H-benzo[d]imidazol-6-yl)-4-methoxypyrrolo[2,1-f][1,2,4]triazin-2-amine [136];
(R)—N-(3,3-difluoro-1-(oxetan-3-yl)piperidin-4-yl)-6-fluoro-5-(1-(2-fluoroethyl)-1H-benzo[d]imidazol-6-yl)-4-methoxypyrrolo[2,1-f][1,2,4]triazin-2-amine [137];
6-fluoro-5-(1-(2-fluoroethyl)-1H-benzo[d]imidazol-6-yl)-4-methoxy-N-(2-oxaspiro[3.5]nonan-7-yl)pyrrolo[2,1-f][1,2,4]triazin-2-amine [138];
3-((5-(1-(2,2-difluoroethyl)-1H-benzo[d]imidazol-6-yl)-4-methoxypyrrolo[2,1-f][1,2,4]triazin-2-yl)amino)-2,2-dimethylpropanenitrile [139];
(1s,4s)-4-((5-(1-(2,2-difluoroethyl)-1H-benzo[d]imidazol-6-yl)-4-methoxypyrrolo[2,1-f][1,2,4]triazin-2-yl)amino)-1-methylcyclohexan-1-ol [140];
(R)—N-(4,4-difluoro-1-methylpyrrolidin-3-yl)-5-(1-(2,2-difluoroethyl)-1H-benzo[d]imidazol-6-yl)-4-methoxypyrrolo[2,1-f][1,2,4]triazin-2-amine [141];
(R)-1-(4-((5-(1-(2,2-difluoroethyl)-1H-benzo[d]imidazol-6-yl)-4-methoxypyrrolo[2,1-f][1,2,4]triazin-2-yl)amino)-3,3-difluoropyrrolidin-1-yl)ethan-1-one [142];
(R)—N-(4,4-difluoro-1-(oxetan-3-yl)pyrrolidin-3-yl)-5-(1-(2,2-difluoroethyl)-1H-benzo[d]imidazol-6-yl)-4-methoxypyrrolo[2,1-f][1,2,4]triazin-2-amine [143];
5-(1-(2,2-difluoroethyl)-1H-benzo[d]imidazol-6-yl)-4-methoxy-N-(1-(oxetan-3-yl)piperidin-4-yl)pyrrolo[2,1-f][1,2,4]triazin-2-amine [144];
5-(1-(2,2-difluoroethyl)-1H-benzo[d]imidazol-6-yl)-N-((3S,4R)-3-fluoro-1-methylpiperidin-4-yl)-4-methoxypyrrolo[2,1-f][1,2,4]triazin-2-amine [145];
5-(1-(2,2-difluoroethyl)-1H-benzo[d]imidazol-6-yl)-N-((3R,4S)-3-fluoro-1-methylpiperidin-4-yl)-4-methoxypyrrolo[2,1-f][1,2,4]triazin-2-amine [146];
5-(1-(2,2-difluoroethyl)-1H-benzo[d]imidazol-6-yl)-N-((3S,4R)-3-fluoro-1-(2-methoxyethyl)piperidin-4-yl)-4-methoxypyrrolo[2,1-f][1,2,4]triazin-2-amine [147];
5-(1-(2,2-difluoroethyl)-1H-benzo[d]imidazol-6-yl)-N-((3R,4R)-3-fluoro-1-(2-methoxyethyl)piperidin-4-yl)-4-methoxypyrrolo[2,1-f][1,2,4]triazin-2-amine [148];
5-(1-(2,2-difluoroethyl)-1H-benzo[d]imidazol-6-yl)-N-((3S,4S)-3-fluoro-1-(2-methoxyethyl)piperidin-4-yl)-4-methoxypyrrolo[2,1-f][1,2,4]triazin-2-amine [149];
5-(1-(2,2-difluoroethyl)-1H-benzo[d]imidazol-6-yl)-N-((3R,4S)-3-fluoro-1-(2-methoxyethyl)piperidin-4-yl)-4-methoxypyrrolo[2,1-f][1,2,4]triazin-2-amine [150];
1-((3R,4S)-4-((5-(1-(2,2-difluoroethyl)-1H-benzo[d]imidazol-6-yl)-4-methoxypyrrolo[2,1-f][1,2,4]triazin-2-yl)amino)-3-fluoropiperidin-1-yl)ethan-1-one [151];
1-((3R,4S)-4-((5-(1-(2,2-difluoroethyl)-1H-benzo[d]imidazol-6-yl)-4-methoxypyrrolo[2,1-f][1,2,4]triazin-2-yl)amino)-3-fluoropiperidin-1-yl)ethan-1-one-2,2,2-d₃ [152];
1-((3R,4S)-4-((5-(1-(2,2-difluoroethyl)-1H-benzo[d]imidazol-6-yl)-4-methoxypyrrolo[2,1-f][1,2,4]triazin-2-yl)amino)-3-fluoropiperidin-1-yl)-2-hydroxyethan-1-one [153];
5-(1-(2,2-difluoroethyl)-1H-benzo[d]imidazol-6-yl)-N-((3S,4R)-3-fluoro-1-(oxetan-3-yl)piperidin-4-yl)-4-methoxypyrrolo[2,1-f][1,2,4]triazin-2-amine [154];
5-(1-(2,2-difluoroethyl)-1H-benzo[d]imidazol-6-yl)-N-((3R,4R)-3-fluoro-1-(oxetan-3-yl)piperidin-4-yl)-4-methoxypyrrolo[2,1-f][1,2,4]triazin-2-amine [155];
5-(1-(2,2-difluoroethyl)-1H-benzo[d]imidazol-6-yl)-N-((3S,4S)-3-fluoro-1-(oxetan-3-yl)piperidin-4-yl)-4-methoxypyrrolo[2,1-f][1,2,4]triazin-2-amine [156];
5-(1-(2,2-difluoroethyl)-1H-benzo[d]imidazol-6-yl)-N-((3S,4R)-3-fluoro-1-(oxetan-3-yl-3-d)piperidin-4-yl)-4-methoxypyrrolo[2,1-f][1,2,4]triazin-2-amine [157];
5-(1-(2,2-difluoroethyl)-1H-benzo[d]imidazol-6-yl)-N-((3R,4R)-3-fluoro-1-(oxetan-3-yl-3-d)piperidin-4-yl)-4-methoxypyrrolo[2,1-f][1,2,4]triazin-2-amine [158];
5-(1-(2,2-difluoroethyl)-1H-benzo[d]imidazol-6-yl)-N-((3S,4S)-3-fluoro-1-(oxetan-3-yl-3-d)piperidin-4-yl)-4-methoxypyrrolo[2,1-f][1,2,4]triazin-2-amine [159];
5-(1-(2,2-difluoroethyl)-1H-benzo[d]imidazol-6-yl)-N-((3R,4S)-3-fluoro-1-(oxetan-3-yl-3-d)piperidin-4-yl)-4-methoxypyrrolo[2,1-f][1,2,4]triazin-2-amine [160];
(R)—N-(3,3-difluoro-1-methylpiperidin-4-yl)-5-(1-(2,2-difluoroethyl)-1H-benzo[d]imidazol-6-yl)-4-methoxypyrrolo[2,1-f][1,2,4]triazin-2-amine [161];
(R)—N-(3,3-difluoro-1-(methyl-d₃)piperidin-4-yl)-5-(1-(2,2-difluoroethyl)-1H-benzo[d]imidazol-6-yl)-4-methoxypyrrolo[2,1-f][1,2,4]triazin-2-amine [162];
(R)—N-(3,3-difluoro-1-(2-methoxyethyl)piperidin-4-yl)-5-(1-(2,2-difluoroethyl)-1H-benzo[d]imidazol-6-yl)-4-methoxypyrrolo[2,1-f][1,2,4]triazin-2-amine [163];
(R)-1-(4-((5-(1-(2,2-difluoroethyl)-1H-benzo[d]imidazol-6-yl)-4-methoxypyrrolo[2,1-f][1,2,4]triazin-2-yl)amino)-3,3-difluoropiperidin-1-yl)ethan-1-one-2,2,2-d₃ [164];
(R)-1-(4-((5-(1-(2,2-difluoroethyl)-1H-benzo[d]imidazol-6-yl)-4-methoxypyrrolo[2,1-f][1,2,4]triazin-2-yl)amino)-3,3-difluoropiperidin-1-yl)-2-hydroxyethan-1-one [165];
(R)—N-(3,3-difluoro-1-(methylsulfonyl)piperidin-4-yl)-5-(1-(2,2-difluoroethyl)-1H-benzo[d]imidazol-6-yl)-4-methoxypyrrolo[2,1-f][1,2,4]triazin-2-amine [166];
(R)—N-(3,3-difluoro-1-(oxetan-3-yl)piperidin-4-yl)-5-(1-(2,2-difluoroethyl)-1H-benzo[d]imidazol-6-yl)-4-methoxypyrrolo[2,1-f][1,2,4]triazin-2-amine [167];
(R)—N-(3,3-difluoro-1-(oxetan-3-yl-3-d)piperidin-4-yl)-5-(1-(2,2-difluoroethyl)-1H-benzo[d]imidazol-6-yl)-4-methoxypyrrolo[2,1-f][1,2,4]triazin-2-amine [168];
5-(1-(2,2-difluoroethyl)-1H-benzo[d]imidazol-6-yl)-4-methoxy-N-(2-oxaspiro[3.5]nonan-7-yl)pyrrolo[2,1-f][1,2,4]triazin-2-amine [169];
3-((5-(1-(2,2-difluoroethyl)-1H-benzo[d]imidazol-6-yl)-6-fluoro-4-methoxypyrrolo[2,1-f][1,2,4]triazin-2-yl)amino)-2,2-dimethylpropanenitrile [170];
(1s,4s)-4-((5-(1-(2,2-difluoroethyl)-1H-benzo[d]imidazol-6-yl)-6-fluoro-4-methoxypyrrolo[2,1-f][1,2,4]triazin-2-yl)amino)-1-methylcyclohexan-1-ol [171];
(1r,4r)-4-((5-(1-(2,2-difluoroethyl)-1H-benzo[d]imidazol-6-yl)-6-fluoro-4-methoxypyrrolo[2,1-f][1,2,4]triazin-2-yl)amino)-1-methylcyclohexan-1-ol [172];
(R)—N-(4,4-difluoro-1-methylpyrrolidin-3-yl)-5-(1-(2,2-difluoroethyl)-1H-benzo[d]imidazol-6-yl)-6-fluoro-4-methoxypyrrolo[2,1-f][1,2,4]triazin-2-amine [173];
(R)-1-(4-((5-(1-(2,2-difluoroethyl)-1H-benzo[d]imidazol-6-yl)-6-fluoro-4-methoxypyrrolo[2,1-f][1,2,4]triazin-2-yl)amino)-3,3-difluoropyrrolidin-1-yl)ethan-1-one [174];
(R)—N-(4,4-difluoro-1-(oxetan-3-yl)pyrrolidin-3-yl)-5-(1-(2,2-difluoroethyl)-1H-benzo[d]imidazol-6-yl)-6-fluoro-4-methoxypyrrolo[2,1-f][1,2,4]triazin-2-amine [175];
5-(1-(2,2-difluoroethyl)-1H-benzo[d]imidazol-6-yl)-6-fluoro-4-methoxy-N-(1-(oxetan-3-yl)piperidin-4-yl)pyrrolo[2,1-f][1,2,4]triazin-2-amine [176];
5-(1-(2,2-difluoroethyl)-1H-benzo[d]imidazol-6-yl)-6-fluoro-N-((3S,4R)-3-fluoro-1-methylpiperidin-4-yl)-4-methoxypyrrolo[2,1-f][1,2,4]triazin-2-amine [177];
5-(1-(2,2-difluoroethyl)-1H-benzo[d]imidazol-6-yl)-6-fluoro-N-((3R,4S)-3-fluoro-1-methylpiperidin-4-yl)-4-methoxypyrrolo[2,1-f][1,2,4]triazin-2-amine [178];
5-(1-(2,2-difluoroethyl)-1H-benzo[d]imidazol-6-yl)-6-fluoro-N-((3S,4R)-3-fluoro-1-(2-methoxyethyl)piperidin-4-yl)-4-methoxypyrrolo[2,1-f][1,2,4]triazin-2-amine [179];
5-(1-(2,2-difluoroethyl)-1H-benzo[d]imidazol-6-yl)-6-fluoro-N-((3R,4R)-3-fluoro-1-(2-methoxyethyl)piperidin-4-yl)-4-methoxypyrrolo[2,1-f][1,2,4]triazin-2-amine [180];
5-(1-(2,2-difluoroethyl)-1H-benzo[d]imidazol-6-yl)-6-fluoro-N-((3S,4S)-3-fluoro-1-(2-methoxyethyl)piperidin-4-yl)-4-methoxypyrrolo[2,1-f][1,2,4]triazin-2-amine [181];
5-(1-(2,2-difluoroethyl)-1H-benzo[d]imidazol-6-yl)-6-fluoro-N-((3R,4S)-3-fluoro-1-(2-methoxyethyl)piperidin-4-yl)-4-methoxypyrrolo[2,1-f][1,2,4]triazin-2-amine [182];
1-((3R,4S)-4-((5-(1-(2,2-difluoroethyl)-1H-benzo[d]imidazol-6-yl)-6-fluoro-4-methoxypyrrolo[2,1-f][1,2,4]triazin-2-yl)amino)-3-fluoropiperidin-1-yl)ethan-1-one [183];
1-((3R,4S)-4-((5-(1-(2,2-difluoroethyl)-1H-benzo[d]imidazol-6-yl)-6-fluoro-4-methoxypyrrolo[2,1-f][1,2,4]triazin-2-yl)amino)-3-fluoropiperidin-1-yl)ethan-1-one-2,2,2-d₃ [184];
1-((3R,4S)-4-((5-(1-(2,2-difluoroethyl)-1H-benzo[d]imidazol-6-yl)-6-fluoro-4-methoxypyrrolo[2,1-f][1,2,4]triazin-2-yl)amino)-3-fluoropiperidin-1-yl)-2-hydroxyethan-1-one [185];
5-(1-(2,2-difluoroethyl)-1H-benzo[d]imidazol-6-yl)-6-fluoro-N-((3S,4R)-3-fluoro-1-(oxetan-3-yl)piperidin-4-yl)-4-methoxypyrrolo[2,1-f][1,2,4]triazin-2-amine [186];
5-(1-(2,2-difluoroethyl)-1H-benzo[d]imidazol-6-yl)-6-fluoro-N-((3R,4R)-3-fluoro-1-(oxetan-3-yl)piperidin-4-yl)-4-methoxypyrrolo[2,1-f][1,2,4]triazin-2-amine [187];
5-(1-(2,2-difluoroethyl)-1H-benzo[d]imidazol-6-yl)-6-fluoro-N-((3S,4S)-3-fluoro-1-(oxetan-3-yl)piperidin-4-yl)-4-methoxypyrrolo[2,1-f][1,2,4]triazin-2-amine [188];
5-(1-(2,2-difluoroethyl)-1H-benzo[d]imidazol-6-yl)-6-fluoro-N-((3R,4S)-3-fluoro-1-(oxetan-3-yl)piperidin-4-yl)-4-methoxypyrrolo[2,1-f][1,2,4]triazin-2-amine [189];
5-(1-(2,2-difluoroethyl)-1H-benzo[d]imidazol-6-yl)-6-fluoro-N-((3S,4R)-3-fluoro-1-(oxetan-3-yl-3-d)piperidin-4-yl)-4-methoxypyrrolo[2,1-f][1,2,4]triazin-2-amine [190];
5-(1-(2,2-difluoroethyl)-1H-benzo[d]imidazol-6-yl)-6-fluoro-N-((3R,4R)-3-fluoro-1-(oxetan-3-yl-3-d)piperidin-4-yl)-4-methoxypyrrolo[2,1-f][1,2,4]triazin-2-amine [191];
5-(1-(2,2-difluoroethyl)-1H-benzo[d]imidazol-6-yl)-6-fluoro-N-((3S,4S)-3-fluoro-1-(oxetan-3-yl-3-d)piperidin-4-yl)-4-methoxypyrrolo[2,1-f][1,2,4]triazin-2-amine [192];
5-(1-(2,2-difluoroethyl)-1H-benzo[d]imidazol-6-yl)-6-fluoro-N-((3R,4S)-3-fluoro-1-(oxetan-3-yl-3-d)piperidin-4-yl)-4-methoxypyrrolo[2,1-f][1,2,4]triazin-2-amine [193];
(R)—N-(3,3-difluoro-1-methylpiperidin-4-yl)-5-(1-(2,2-difluoroethyl)-1H-benzo[d]imidazol-6-yl)-6-fluoro-4-methoxypyrrolo[2,1-f][1,2,4]triazin-2-amine [194];
(R)—N-(3,3-difluoro-1-(methyl-d₃)piperidin-4-yl)-5-(1-(2,2-difluoroethyl)-1H-benzo[d]imidazol-6-yl)-6-fluoro-4-methoxypyrrolo[2,1-f][1,2,4]triazin-2-amine [195];
(R)—N-(3,3-difluoro-1-(2-methoxyethyl)piperidin-4-yl)-5-(1-(2,2-difluoroethyl)-1H-benzo[d]imidazol-6-yl)-6-fluoro-4-methoxypyrrolo[2,1-f][1,2,4]triazin-2-amine [196];
(R)-1-(4-((5-(1-(2,2-difluoroethyl)-1H-benzo[d]imidazol-6-yl)-6-fluoro-4-methoxypyrrolo[2,1-f][1,2,4]triazin-2-yl)amino)-3,3-difluoropiperidin-1-yl)ethan-1-one [197];
(R)-1-(4-((5-(1-(2,2-difluoroethyl)-1H-benzo[d]imidazol-6-yl)-6-fluoro-4-methoxypyrrolo[2,1-f][1,2,4]triazin-2-yl)amino)-3,3-difluoropiperidin-1-yl)ethan-1-one-2,2,2-d₃ [198];
(R)-1-(4-((5-(1-(2,2-difluoroethyl)-1H-benzo[d]imidazol-6-yl)-6-fluoro-4-methoxypyrrolo[2,1-f][1,2,4]triazin-2-yl)amino)-3,3-difluoropiperidin-1-yl)-2-hydroxyethan-1-one [199]; and
(R)—N-(3,3-difluoro-1-(methylsulfonyl)piperidin-4-yl)-5-(1-(2,2-difluoroethyl)-1H-benzo[d]imidazol-6-yl)-6-fluoro-4-methoxypyrrolo[2,1-f][1,2,4]triazin-2-amine [200]; or a pharmaceutically acceptable salt thereof.

19. The compound of claim 1, wherein the compound of Formula I is selected from the group consisting of
(R)—N-(3,3-difluoro-1-(oxetan-3-yl)piperidin-4-yl)-5-(1-(2,2-difluoroethyl)-1H-benzo[d]imidazol-6-yl)-6-fluoro-4-methoxypyrrolo[2,1-f][1,2,4]triazin-2-amine [201];
(R)—N-(3,3-difluoro-1-(oxetan-3-yl-3-d)piperidin-4-yl)-5-(1-(2,2-difluoroethyl)-1H-benzo[d]imidazol-6-yl)-6-fluoro-4-methoxypyrrolo[2,1-f][1,2,4]triazin-2-amine [202];
5-(1-(2,2-difluoroethyl)-1H-benzo[d]imidazol-6-yl)-6-fluoro-4-methoxy-N-(2-oxaspiro[3.5]nonan-7-yl)pyrrolo[2,1-f][1,2,4]triazin-2-amine [203];
3-((5-(4-fluoro-1-(2-fluoroethyl)-1H-benzo[d]imidazol-6-yl)-4-methoxypyrrolo[2,1-f][1,2,4]triazin-2-yl)amino)-2,2-dimethylpropanenitrile [204];
(1s,4s)-4-((5-(4-fluoro-1-(2-fluoroethyl)-1H-benzo[d]imidazol-6-yl)-4-methoxypyrrolo[2,1-f][1,2,4]triazin-2-yl)amino)-1-methylcyclohexan-1-ol [205];
(1r,4r)-4-((5-(4-fluoro-1-(2-fluoroethyl)-1H-benzo[d]imidazol-6-yl)-4-methoxypyrrolo[2,1-f][1,2,4]triazin-2-yl)amino)-1-methylcyclohexan-1-ol [206];
(R)—N-(4,4-difluoro-1-methylpyrrolidin-3-yl)-5-(4-fluoro-1-(2-fluoroethyl)-1H-benzo[d]imidazol-6-yl)-4-methoxypyrrolo[2,1-f][1,2,4]triazin-2-amine [207];
(R)-1-(3,3-difluoro-4-((5-(4-fluoro-1-(2-fluoroethyl)-1H-benzo[d]imidazol-6-yl)-4-methoxypyrrolo[2,1-f][1,2,4]triazin-2-yl)amino)pyrrolidin-1-yl)ethan-1-one [208];
(R)—N-(4,4-difluoro-1-(oxetan-3-yl)pyrrolidin-3-yl)-5-(4-fluoro-1-(2-fluoroethyl)-1H-benzo[d]imidazol-6-yl)-4-methoxypyrrolo[2,1-f][1,2,4]triazin-2-amine [209];
5-(4-fluoro-1-(2-fluoroethyl)-1H-benzo[d]imidazol-6-yl)-4-methoxy-N-(1-(oxetan-3-yl)piperidin-4-yl)pyrrolo[2,1-f][1,2,4]triazin-2-amine [210];
5-(4-fluoro-1-(2-fluoroethyl)-1H-benzo[d]imidazol-6-yl)-N-((3S,4R)-3-fluoro-1-methylpiperidin-4-yl)-4-methoxypyrrolo[2,1-f][1,2,4]triazin-2-amine [211];
5-(4-fluoro-1-(2-fluoroethyl)-1H-benzo[d]imidazol-6-yl)-N-((3R,4S)-3-fluoro-1-methylpiperidin-4-yl)-4-methoxypyrrolo[2,1-f][1,2,4]triazin-2-amine [212];
1-((3R,4S)-3-fluoro-4-((5-(4-fluoro-1-(2-fluoroethyl)-1H-benzo[d]imidazol-6-yl)-4-methoxypyrrolo[2,1-f][1,2,4]triazin-2-yl)amino)piperidin-1-yl)ethan-1-one [213];
1-((3R,4S)-3-fluoro-4-((5-(4-fluoro-1-(2-fluoroethyl)-1H-benzo[d]imidazol-6-yl)-4-methoxypyrrolo[2,1-f][1,2,4]triazin-2-yl)amino)piperidin-1-yl)ethan-1-one-2,2,2-d₃ [214];
1-((3R,4S)-3-fluoro-4-((5-(4-fluoro-1-(2-fluoroethyl)-1H-benzo[d]imidazol-6-yl)-4-methoxypyrrolo[2,1-f][1,2,4]triazin-2-yl)amino)piperidin-1-yl)-2-hydroxyethan-1-one [215];
5-(4-fluoro-1-(2-fluoroethyl)-1H-benzo[d]imidazol-6-yl)-N-((3S,4R)-3-fluoro-1-(oxetan-3-yl)piperidin-4-yl)-4-methoxypyrrolo[2,1-f][1,2,4]triazin-2-amine [216];
5-(4-fluoro-1-(2-fluoroethyl)-1H-benzo[d]imidazol-6-yl)-N-((3R,4R)-3-fluoro-1-(oxetan-3-yl)piperidin-4-yl)-4-methoxypyrrolo[2,1-f][1,2,4]triazin-2-amine [217];
5-(4-fluoro-1-(2-fluoroethyl)-1H-benzo[d]imidazol-6-yl)-N-((3S,4S)-3-fluoro-1-(oxetan-3-yl)piperidin-4-yl)-4-methoxypyrrolo[2,1-f][1,2,4]triazin-2-amine [218];
5-(4-fluoro-1-(2-fluoroethyl)-1H-benzo[d]imidazol-6-yl)-N-((3R,4S)-3-fluoro-1-(oxetan-3-yl)piperidin-4-yl)-4-methoxypyrrolo[2,1-f][1,2,4]triazin-2-amine [219];
(R)—N-(3,3-difluoro-1-methylpiperidin-4-yl)-5-(4-fluoro-1-(2-fluoroethyl)-1H-benzo[d]imidazol-6-yl)-4-methoxypyrrolo[2,1-f][1,2,4]triazin-2-amine [220];
(R)—N-(3,3-difluoro-1-(methyl-d₃)piperidin-4-yl)-5-(4-fluoro-1-(2-fluoroethyl)-1H-benzo[d]imidazol-6-yl)-4-methoxypyrrolo[2,1-f][1,2,4]triazin-2-amine [221];
(R)-1-(3,3-difluoro-4-((5-(4-fluoro-1-(2-fluoroethyl)-1H-benzo[d]imidazol-6-yl)-4-methoxypyrrolo[2,1-f][1,2,4]triazin-2-yl)amino)piperidin-1-yl)ethan-1-one [222];
(R)-1-(3,3-difluoro-4-((5-(4-fluoro-1-(2-fluoroethyl)-1H-benzo[d]imidazol-6-yl)-4-methoxypyrrolo[2,1-f][1,2,4]triazin-2-yl)amino)piperidin-1-yl)ethan-1-one-2,2,2-d₃ [223];
(R)-1-(3,3-difluoro-4-((5-(4-fluoro-1-(2-fluoroethyl)-1H-benzo[d]imidazol-6-yl)-4-methoxypyrrolo[2,1-f][1,2,4]triazin-2-yl)amino)piperidin-1-yl)-2-hydroxyethan-1-one [224];
(R)—N-(3,3-difluoro-1-(methylsulfonyl)piperidin-4-yl)-5-(4-fluoro-1-(2-fluoroethyl)-1H-benzo[d]imidazol-6-yl)-4-methoxypyrrolo[2,1-f][1,2,4]triazin-2-amine [225];
(R)—N-(3,3-difluoro-1-(oxetan-3-yl)piperidin-4-yl)-5-(4-fluoro-1-(2-fluoroethyl)-1H-benzo[d]imidazol-6-yl)-4-methoxypyrrolo[2,1-f][1,2,4]triazin-2-amine [226];
5-(4-fluoro-1-(2-fluoroethyl)-1H-benzo[d]imidazol-6-yl)-4-methoxy-N-(2-oxaspiro[3.5]nonan-7-yl)pyrrolo[2,1-f][1,2,4]triazin-2-amine [227];
3-((6-fluoro-5-(4-fluoro-1-(2-fluoroethyl)-1H-benzo[d]imidazol-6-yl)-4-methoxypyrrolo[2,1-f][1,2,4]triazin-2-yl)amino)-2,2-dimethylpropanenitrile [228];
(1s,4s)-4-((6-fluoro-5-(4-fluoro-1-(2-fluoroethyl)-1H-benzo[d]imidazol-6-yl)-4-methoxypyrrolo[2,1-f][1,2,4]triazin-2-yl)amino)-1-methylcyclohexan-1-ol [229];
(1r,4r)-4-((6-fluoro-5-(4-fluoro-1-(2-fluoroethyl)-1H-benzo[d]imidazol-6-yl)-4-methoxypyrrolo[2,1-f][1,2,4]triazin-2-yl)amino)-1-methylcyclohexan-1-ol [230];
(R)—N-(4,4-difluoro-1-methylpyrrolidin-3-yl)-6-fluoro-5-(4-fluoro-1-(2-fluoroethyl)-1H-benzo[d]imidazol-6-yl)-4-methoxypyrrolo[2,1-f][1,2,4]triazin-2-amine [231];
(R)-1-(3,3-difluoro-4-((6-fluoro-5-(4-fluoro-1-(2-fluoroethyl)-1H-benzo[d]imidazol-6-yl)-4-methoxypyrrolo[2,1-f][1,2,4]triazin-2-yl)amino)pyrrolidin-1-yl)ethan-1-one [232];
(R)—N-(4,4-difluoro-1-(oxetan-3-yl)pyrrolidin-3-yl)-6-fluoro-5-(4-fluoro-1-(2-fluoroethyl)-1H-benzo[d]imidazol-6-yl)-4-methoxypyrrolo[2,1-f][1,2,4]triazin-2-amine [233];
6-fluoro-5-(4-fluoro-1-(2-fluoroethyl)-1H-benzo[d]imidazol-6-yl)-4-methoxy-N-(1-(oxetan-3-yl)piperidin-4-yl)pyrrolo[2,1-f][1,2,4]triazin-2-amine [234];
6-fluoro-5-(4-fluoro-1-(2-fluoroethyl)-1H-benzo[d]imidazol-6-yl)-N-((3S,4R)-3-fluoro-1-methylpiperidin-4-yl)-4-methoxypyrrolo[2,1-f][1,2,4]triazin-2-amine [235];
6-fluoro-5-(4-fluoro-1-(2-fluoroethyl)-1H-benzo[d]imidazol-6-yl)-N-((3R,4S)-3-fluoro-1-methylpiperidin-4-yl)-4-methoxypyrrolo[2,1-f][1,2,4]triazin-2-amine [236];
1-((3R,4S)-3-fluoro-4-((6-fluoro-5-(4-fluoro-1-(2-fluoroethyl)-1H-benzo[d]imidazol-6-yl)-4-methoxypyrrolo[2,1-f][1,2,4]triazin-2-yl)amino)piperidin-1-yl)ethan-1-one [237];
1-((3R,4S)-3-fluoro-4-((6-fluoro-5-(4-fluoro-1-(2-fluoroethyl)-1H-benzo[d]imidazol-6-yl)-4-methoxypyrrolo[2,1-f][1,2,4]triazin-2-yl)amino)piperidin-1-yl)ethan-1-one-2,2,2-d₃ [238];
1-((3R,4S)-3-fluoro-4-((6-fluoro-5-(4-fluoro-1-(2-fluoroethyl)-1H-benzo[d]imidazol-6-yl)-4-methoxypyrrolo[2,1-f][1,2,4]triazin-2-yl)amino)piperidin-1-yl)-2-hydroxyethan-1-one [239];
6-fluoro-5-(4-fluoro-1-(2-fluoroethyl)-1H-benzo[d]imidazol-6-yl)-N-((3S,4R)-3-fluoro-1-(oxetan-3-yl)piperidin-4-yl)-4-methoxypyrrolo[2,1-f][1,2,4]triazin-2-amine [240];
6-fluoro-5-(4-fluoro-1-(2-fluoroethyl)-1H-benzo[d]imidazol-6-yl)-N-((3R,4R)-3-fluoro-1-(oxetan-3-yl)piperidin-4-yl)-4-methoxypyrrolo[2,1-f][1,2,4]triazin-2-amine [241];
6-fluoro-5-(4-fluoro-1-(2-fluoroethyl)-1H-benzo[d]imidazol-6-yl)-N-((3S,4S)-3-fluoro-1-(oxetan-3-yl)piperidin-4-yl)-4-methoxypyrrolo[2,1-f][1,2,4]triazin-2-amine [242];
6-fluoro-5-(4-fluoro-1-(2-fluoroethyl)-1H-benzo[d]imidazol-6-yl)-N-((3R,4S)-3-fluoro-1-(oxetan-3-yl)piperidin-4-yl)-4-methoxypyrrolo[2,1-f][1,2,4]triazin-2-amine [243];
(R)—N-(3,3-difluoro-1-methylpiperidin-4-yl)-6-fluoro-5-(4-fluoro-1-(2-fluoroethyl)-1H-benzo[d]imidazol-6-yl)-4-methoxypyrrolo[2,1-f][1,2,4]triazin-2-amine [244];
(R)—N-(3,3-difluoro-1-(methyl-d₃)piperidin-4-yl)-6-fluoro-5-(4-fluoro-1-(2-fluoroethyl)-1H-benzo[d]imidazol-6-yl)-4-methoxypyrrolo[2,1-f][1,2,4]triazin-2-amine [245];
(R)-1-(3,3-difluoro-4-((6-fluoro-5-(4-fluoro-1-(2-fluoroethyl)-1H-benzo[d]imidazol-6-yl)-4-methoxypyrrolo[2,1-f][1,2,4]triazin-2-yl)amino)piperidin-1-yl)ethan-1-one [246];
(R)-1-(3,3-difluoro-4-((6-fluoro-5-(4-fluoro-1-(2-fluoroethyl)-1H-benzo[d]imidazol-6-yl)-4-methoxypyrrolo[2,1-f][1,2,4]triazin-2-yl)amino)piperidin-1-yl)ethan-1-one-2,2,2-d₃ [247];
(R)-1-(3,3-difluoro-4-((6-fluoro-5-(4-fluoro-1-(2-fluoroethyl)-1H-benzo[d]imidazol-6-yl)-4-methoxypyrrolo[2,1-f][1,2,4]triazin-2-yl)amino)piperidin-1-yl)-2-hydroxyethan-1-one [248];
(R)—N-(3,3-difluoro-1-(methylsulfonyl)piperidin-4-yl)-6-fluoro-5-(4-fluoro-1-(2-fluoroethyl)-1H-benzo[d]imidazol-6-yl)-4-methoxypyrrolo[2,1-f][1,2,4]triazin-2-amine [249];
(R)—N-(3,3-difluoro-1-(oxetan-3-yl)piperidin-4-yl)-6-fluoro-5-(4-fluoro-1-(2-fluoroethyl)-1H-benzo[d]imidazol-6-yl)-4-methoxypyrrolo[2,1-f][1,2,4]triazin-2-amine [250];
6-fluoro-5-(4-fluoro-1-(2-fluoroethyl)-1H-benzo[d]imidazol-6-yl)-4-methoxy-N-(2-oxaspiro[3.5]nonan-7-yl)pyrrolo[2,1-f][1,2,4]triazin-2-amine [251];
3-((5-(1-(2,2-difluoroethyl)-4-fluoro-1H-benzo[d]imidazol-6-yl)-4-methoxypyrrolo[2,1-f][1,2,4]triazin-2-yl)amino)-2,2-dimethylpropanenitrile [252];
(1s,4s)-4-((5-(1-(2,2-difluoroethyl)-4-fluoro-1H-benzo[d]imidazol-6-yl)-4-methoxypyrrolo[2,1-f][1,2,4]triazin-2-yl)amino)-1-methylcyclohexan-1-ol [253];
(1r,4r)-4-((5-(1-(2,2-difluoroethyl)-4-fluoro-1H-benzo[d]imidazol-6-yl)-4-methoxypyrrolo[2,1-f][1,2,4]triazin-2-yl)amino)-1-methylcyclohexan-1-ol [254];
(R)—N-(4,4-difluoro-1-methylpyrrolidin-3-yl)-5-(1-(2,2-difluoroethyl)-4-fluoro-1H-benzo[d]imidazol-6-yl)-4-methoxypyrrolo[2,1-f][1,2,4]triazin-2-amine [255];
(R)-1-(4-((5-(1-(2,2-difluoroethyl)-4-fluoro-1H-benzo[d]imidazol-6-yl)-4-methoxypyrrolo[2,1-f][1,2,4]triazin-2-yl)amino)-3,3-difluoropyrrolidin-1-yl)ethan-1-one [256];
(R)—N-(4,4-difluoro-1-(oxetan-3-yl)pyrrolidin-3-yl)-5-(1-(2,2-difluoroethyl)-4-fluoro-1H-benzo[d]imidazol-6-yl)-4-methoxypyrrolo[2,1-f][1,2,4]triazin-2-amine [257];
5-(1-(2,2-difluoroethyl)-4-fluoro-1H-benzo[d]imidazol-6-yl)-4-methoxy-N-(1-(oxetan-3-yl)piperidin-4-yl)pyrrolo[2,1-f][1,2,4]triazin-2-amine [258];
5-(1-(2,2-difluoroethyl)-4-fluoro-1H-benzo[d]imidazol-6-yl)-N-((3S,4R)-3-fluoro-1-methylpiperidin-4-yl)-4-methoxypyrrolo[2,1-f][1,2,4]triazin-2-amine [259];
5-(1-(2,2-difluoroethyl)-4-fluoro-1H-benzo[d]imidazol-6-yl)-N-((3R,4S)-3-fluoro-1-methylpiperidin-4-yl)-4-methoxypyrrolo[2,1-f][1,2,4]triazin-2-amine [260];
5-(1-(2,2-difluoroethyl)-4-fluoro-1H-benzo[d]imidazol-6-yl)-N-((3S,4R)-3-fluoro-1-(2-methoxyethyl)piperidin-4-yl)-4-methoxypyrrolo[2,1-f][1,2,4]triazin-2-amine [261];
5-(1-(2,2-difluoroethyl)-4-fluoro-1H-benzo[d]imidazol-6-yl)-N-((3R,4R)-3-fluoro-1-(2-methoxyethyl)piperidin-4-yl)-4-methoxypyrrolo[2,1-f][1,2,4]triazin-2-amine [262];
5-(1-(2,2-difluoroethyl)-4-fluoro-1H-benzo[d]imidazol-6-yl)-N-((3S,4S)-3-fluoro-1-(2-methoxyethyl)piperidin-4-yl)-4-methoxypyrrolo[2,1-f][1,2,4]triazin-2-amine [263];
5-(1-(2,2-difluoroethyl)-4-fluoro-1H-benzo[d]imidazol-6-yl)-N-((3R,4S)-3-fluoro-1-(2-methoxyethyl)piperidin-4-yl)-4-methoxypyrrolo[2,1-f][1,2,4]triazin-2-amine [264];
1-((3R,4S)-4-((5-(1-(2,2-difluoroethyl)-4-fluoro-1H-benzo[d]imidazol-6-yl)-4-methoxypyrrolo[2,1-f][1,2,4]triazin-2-yl)amino)-3-fluoropiperidin-1-yl)ethan-1-one [265];
1-((3R,4S)-4-((5-(1-(2,2-difluoroethyl)-4-fluoro-1H-benzo[d]imidazol-6-yl)-4-methoxypyrrolo[2,1-f][1,2,4]triazin-2-yl)amino)-3-fluoropiperidin-1-yl)ethan-1-one-2,2,2-d₃ [266];
1-((3R,4S)-4-((5-(1-(2,2-difluoroethyl)-4-fluoro-1H-benzo[d]imidazol-6-yl)-4-methoxypyrrolo[2,1-f][1,2,4]triazin-2-yl)amino)-3-fluoropiperidin-1-yl)-2-hydroxyethan-1-one [267];
5-(1-(2,2-difluoroethyl)-4-fluoro-1H-benzo[d]imidazol-6-yl)-N-((3S,4R)-3-fluoro-1-(oxetan-3-yl)piperidin-4-yl)-4-methoxypyrrolo[2,1-f][1,2,4]triazin-2-amine [268];
5-(1-(2,2-difluoroethyl)-4-fluoro-1H-benzo[d]imidazol-6-yl)-N-((3R,4R)-3-fluoro-1-(oxetan-3-yl)piperidin-4-yl)-4-methoxypyrrolo[2,1-f][1,2,4]triazin-2-amine [269];
5-(1-(2,2-difluoroethyl)-4-fluoro-1H-benzo[d]imidazol-6-yl)-N-((3S,4S)-3-fluoro-1-(oxetan-3-yl)piperidin-4-yl)-4-methoxypyrrolo[2,1-f][1,2,4]triazin-2-amine [270];
5-(1-(2,2-difluoroethyl)-4-fluoro-1H-benzo[d]imidazol-6-yl)-N-((3R,4S)-3-fluoro-1-(oxetan-3-yl)piperidin-4-yl)-4-methoxypyrrolo[2,1-f][1,2,4]triazin-2-amine [271];
5-(1-(2,2-difluoroethyl)-4-fluoro-1H-benzo[d]imidazol-6-yl)-N-((3S,4R)-3-fluoro-1-(oxetan-3-yl-3-d)piperidin-4-yl)-4-methoxypyrrolo[2,1-f][1,2,4]triazin-2-amine [272];
5-(1-(2,2-difluoroethyl)-4-fluoro-1H-benzo[d]imidazol-6-yl)-N-((3R,4R)-3-fluoro-1-(oxetan-3-yl-3-d)piperidin-4-yl)-4-methoxypyrrolo[2,1-f][1,2,4]triazin-2-amine [273];
5-(1-(2,2-difluoroethyl)-4-fluoro-1H-benzo[d]imidazol-6-yl)-N-((3S,4S)-3-fluoro-1-(oxetan-3-yl-3-d)piperidin-4-yl)-4-methoxypyrrolo[2,1-f][1,2,4]triazin-2-amine [274];
5-(1-(2,2-difluoroethyl)-4-fluoro-1H-benzo[d]imidazol-6-yl)-N-((3R,4S)-3-fluoro-1-(oxetan-3-yl-3-d)piperidin-4-yl)-4-methoxypyrrolo[2,1-f][1,2,4]triazin-2-amine [275];
(R)—N-(3,3-difluoro-1-methylpiperidin-4-yl)-5-(1-(2,2-difluoroethyl)-4-fluoro-1H-benzo[d]imidazol-6-yl)-4-methoxypyrrolo[2,1-f][1,2,4]triazin-2-amine [276];
(R)—N-(3,3-difluoro-1-(methyl-d₃)piperidin-4-yl)-5-(1-(2,2-difluoroethyl)-4-fluoro-1H-benzo[d]imidazol-6-yl)-4-methoxypyrrolo[2,1-f][1,2,4]triazin-2-amine [277];
(R)—N-(3,3-difluoro-1-(2-methoxyethyl)piperidin-4-yl)-5-(1-(2,2-difluoroethyl)-4-fluoro-1H-benzo[d]imidazol-6-yl)-4-methoxypyrrolo[2,1-f][1,2,4]triazin-2-amine [278];
(R)-1-(4-((5-(1-(2,2-difluoroethyl)-4-fluoro-1H-benzo[d]imidazol-6-yl)-4-methoxypyrrolo[2,1-f][1,2,4]triazin-2-yl)amino)-3,3-difluoropiperidin-1-yl)ethan-1-one [279];
(R)-1-(4-((5-(1-(2,2-difluoroethyl)-4-fluoro-1H-benzo[d]imidazol-6-yl)-4-methoxypyrrolo[2,1-f][1,2,4]triazin-2-yl)amino)-3,3-difluoropiperidin-1-yl)ethan-1-one-2,2,2-d₃ [280];
(R)-1-(4-((5-(1-(2,2-difluoroethyl)-4-fluoro-1H-benzo[d]imidazol-6-yl)-4-methoxypyrrolo[2,1-f][1,2,4]triazin-2-yl)amino)-3,3-difluoropiperidin-1-yl)-2-hydroxyethan-1-one [281];
(R)—N-(3,3-difluoro-1-(methylsulfonyl)piperidin-4-yl)-5-(1-(2,2-difluoroethyl)-4-fluoro-1H-benzo[d]imidazol-6-yl)-4-methoxypyrrolo[2,1-f][1,2,4]triazin-2-amine [282];
(R)—N-(3,3-difluoro-1-(oxetan-3-yl)piperidin-4-yl)-5-(1-(2,2-difluoroethyl)-4-fluoro-1H-benzo[d]imidazol-6-yl)-4-methoxypyrrolo[2,1-f][1,2,4]triazin-2-amine [283];
(R)—N-(3,3-difluoro-1-(oxetan-3-yl-3-d)piperidin-4-yl)-5-(1-(2,2-difluoroethyl)-4-fluoro-1H-benzo[d]imidazol-6-yl)-4-methoxypyrrolo[2,1-f][1,2,4]triazin-2-amine [284];
5-(1-(2,2-difluoroethyl)-4-fluoro-1H-benzo[d]imidazol-6-yl)-4-methoxy-N-(2-oxaspiro[3.5]nonan-7-yl)pyrrolo[2,1-f][1,2,4]triazin-2-amine [285];
3-((5-(1-(2,2-difluoroethyl)-4-fluoro-1H-benzo[d]imidazol-6-yl)-6-fluoro-4-methoxypyrrolo[2,1-f][1,2,4]triazin-2-yl)amino)-2,2-dimethylpropanenitrile [286];
(1s,4s)-4-((5-(1-(2,2-difluoroethyl)-4-fluoro-1H-benzo[d]imidazol-6-yl)-6-fluoro-4-methoxypyrrolo[2,1-f][1,2,4]triazin-2-yl)amino)-1-methylcyclohexan-1-ol [287];
(1r,4r)-4-((5-(1-(2,2-difluoroethyl)-4-fluoro-1H-benzo[d]imidazol-6-yl)-6-fluoro-4-methoxypyrrolo[2,1-f][1,2,4]triazin-2-yl)amino)-1-methylcyclohexan-1-ol [288];
(R)—N-(4,4-difluoro-1-methylpyrrolidin-3-yl)-5-(1-(2,2-difluoroethyl)-4-fluoro-1H-benzo[d]imidazol-6-yl)-6-fluoro-4-methoxypyrrolo[2,1-f][1,2,4]triazin-2-amine [289];
(R)-1-(4-((5-(1-(2,2-difluoroethyl)-4-fluoro-1H-benzo[d]imidazol-6-yl)-6-fluoro-4-methoxypyrrolo[2,1-f][1,2,4]triazin-2-yl)amino)-3,3-difluoropyrrolidin-1-yl)ethan-1-one [290];
(R)—N-(4,4-difluoro-1-(oxetan-3-yl)pyrrolidin-3-yl)-5-(1-(2,2-difluoroethyl)-4-fluoro-1H-benzo[d]imidazol-6-yl)-6-fluoro-4-methoxypyrrolo[2,1-f][1,2,4]triazin-2-amine [291];
5-(1-(2,2-difluoroethyl)-4-fluoro-1H-benzo[d]imidazol-6-yl)-6-fluoro-4-methoxy-N-(1-(oxetan-3-yl)piperidin-4-yl)pyrrolo[2,1-f][1,2,4]triazin-2-amine [292];
5-(1-(2,2-difluoroethyl)-4-fluoro-1H-benzo[d]imidazol-6-yl)-6-fluoro-N-((3S,4R)-3-fluoro-1-methylpiperidin-4-yl)-4-methoxypyrrolo[2,1-f][1,2,4]triazin-2-amine [293];
5-(1-(2,2-difluoroethyl)-4-fluoro-1H-benzo[d]imidazol-6-yl)-6-fluoro-N-((3R,4S)-3-fluoro-1-methylpiperidin-4-yl)-4-methoxypyrrolo[2,1-f][1,2,4]triazin-2-amine [294];
5-(1-(2,2-difluoroethyl)-4-fluoro-1H-benzo[d]imidazol-6-yl)-6-fluoro-N-((3S,4R)-3-fluoro-1-(2-methoxyethyl)piperidin-4-yl)-4-methoxypyrrolo[2,1-f][1,2,4]triazin-2-amine [295];
5-(1-(2,2-difluoroethyl)-4-fluoro-1H-benzo[d]imidazol-6-yl)-6-fluoro-N-((3R,4R)-3-fluoro-1-(2-methoxyethyl)piperidin-4-yl)-4-methoxypyrrolo[2,1-f][1,2,4]triazin-2-amine [296];
5-(1-(2,2-difluoroethyl)-4-fluoro-1H-benzo[d]imidazol-6-yl)-6-fluoro-N-((3S,4S)-3-fluoro-1-(2-methoxyethyl)piperidin-4-yl)-4-methoxypyrrolo[2,1-f][1,2,4]triazin-2-amine [297];
5-(1-(2,2-difluoroethyl)-4-fluoro-1H-benzo[d]imidazol-6-yl)-6-fluoro-N-((3R,4S)-3-fluoro-1-(2-methoxyethyl)piperidin-4-yl)-4-methoxypyrrolo[2,1-f][1,2,4]triazin-2-amine [298];
1-((3R,4S)-4-((5-(1-(2,2-difluoroethyl)-4-fluoro-1H-benzo[d]imidazol-6-yl)-6-fluoro-4-methoxypyrrolo[2,1-f][1,2,4]triazin-2-yl)amino)-3-fluoropiperidin-1-yl)ethan-1-one [299]; and
1-((3R,4S)-4-((5-(1-(2,2-difluoroethyl)-4-fluoro-1H-benzo[d]imidazol-6-yl)-6-fluoro-4-methoxypyrrolo[2,1-f][1,2,4]triazin-2-yl)amino)-3-fluoropiperidin-1-yl)ethan-1-one-2,2,2-d₃ [300]; or a pharmaceutically acceptable salt thereof.

20. The compound of claim 1, wherein the compound of Formula I is selected from the group consisting of
1-((3R,4S)-4-((5-(1-(2,2-difluoroethyl)-4-fluoro-1H-benzo[d]imidazol-6-yl)-6-fluoro-4-methoxypyrrolo[2,1-f][1,2,4]triazin-2-yl)amino)-3-fluoropiperidin-1-yl)-2-hydroxyethan-1-one [301];
5-(1-(2,2-difluoroethyl)-4-fluoro-1H-benzo[d]imidazol-6-yl)-6-fluoro-N-((3S,4R)-3-fluoro-1-(oxetan-3-yl)piperidin-4-yl)-4-methoxypyrrolo[2,1-f][1,2,4]triazin-2-amine [302];
5-(1-(2,2-difluoroethyl)-4-fluoro-1H-benzo[d]imidazol-6-yl)-6-fluoro-N-((3R,4R)-3-fluoro-1-(oxetan-3-yl)piperidin-4-yl)-4-methoxypyrrolo[2,1-f][1,2,4]triazin-2-amine [303];
5-(1-(2,2-difluoroethyl)-4-fluoro-1H-benzo[d]imidazol-6-yl)-6-fluoro-N-((3S,4S)-3-fluoro-1-(oxetan-3-yl)piperidin-4-yl)-4-methoxypyrrolo[2,1-f][1,2,4]triazin-2-amine [304];
5-(1-(2,2-difluoroethyl)-4-fluoro-1H-benzo[d]imidazol-6-yl)-6-fluoro-N-((3R,4S)-3-fluoro-1-(oxetan-3-yl)piperidin-4-yl)-4-methoxypyrrolo[2,1-f][1,2,4]triazin-2-amine [305];
5-(1-(2,2-difluoroethyl)-4-fluoro-1H-benzo[d]imidazol-6-yl)-6-fluoro-N-((3S,4R)-3-fluoro-1-(oxetan-3-yl-3-d)piperidin-4-yl)-4-methoxypyrrolo[2,1-f][1,2,4]triazin-2-amine [306];
5-(1-(2,2-difluoroethyl)-4-fluoro-1H-benzo[d]imidazol-6-yl)-6-fluoro-N-((3R,4R)-3-fluoro-1-(oxetan-3-yl-3-d)piperidin-4-yl)-4-methoxypyrrolo[2,1-f][1,2,4]triazin-2-amine [307];
5-(1-(2,2-difluoroethyl)-4-fluoro-1H-benzo[d]imidazol-6-yl)-6-fluoro-N-((3S,4S)-3-fluoro-1-(oxetan-3-yl-3-d)piperidin-4-yl)-4-methoxypyrrolo[2,1-f][1,2,4]triazin-2-amine [308];
5-(1-(2,2-difluoroethyl)-4-fluoro-1H-benzo[d]imidazol-6-yl)-6-fluoro-N-((3R,4S)-3-fluoro-1-(oxetan-3-yl-3-d)piperidin-4-yl)-4-methoxypyrrolo[2,1-f][1,2,4]triazin-2-amine [309];
(R)—N-(3,3-difluoro-1-methylpiperidin-4-yl)-5-(1-(2,2-difluoroethyl)-4-fluoro-1H-benzo[d]imidazol-6-yl)-6-fluoro-4-methoxypyrrolo[2,1-f][1,2,4]triazin-2-amine [310];
(R)—N-(3,3-difluoro-1-(methyl-d₃)piperidin-4-yl)-5-(1-(2,2-difluoroethyl)-4-fluoro-1H-benzo[d]imidazol-6-yl)-6-fluoro-4-methoxypyrrolo[2,1-f][1,2,4]triazin-2-amine [311];
(R)—N-(3,3-difluoro-1-(2-methoxyethyl)piperidin-4-yl)-5-(1-(2,2-difluoroethyl)-4-fluoro-1H-benzo[d]imidazol-6-yl)-6-fluoro-4-methoxypyrrolo[2,1-f][1,2,4]triazin-2-amine [312];
(R)-1-(4-((5-(1-(2,2-difluoroethyl)-4-fluoro-1H-benzo[d]imidazol-6-yl)-6-fluoro-4-methoxypyrrolo[2,1-f][1,2,4]triazin-2-yl)amino)-3,3-difluoropiperidin-1-yl)ethan-1-one [313];
(R)-1-(4-((5-(1-(2,2-difluoroethyl)-4-fluoro-1H-benzo[d]imidazol-6-yl)-6-fluoro-4-methoxypyrrolo[2,1-f][1,2,4]triazin-2-yl)amino)-3,3-difluoropiperidin-1-yl)ethan-1-one-2,2,2-d₃ [314];
(R)-1-(4-((5-(1-(2,2-difluoroethyl)-4-fluoro-1H-benzo[d]imidazol-6-yl)-6-fluoro-4-methoxypyrrolo[2,1-f][1,2,4]triazin-2-yl)amino)-3,3-difluoropiperidin-1-yl)-2-hydroxyethan-1-one [315];
(R)—N-(3,3-difluoro-1-(methylsulfonyl)piperidin-4-yl)-5-(1-(2,2-difluoroethyl)-4-fluoro-1H-benzo[d]imidazol-6-yl)-6-fluoro-4-methoxypyrrolo[2,1-f][1,2,4]triazin-2-amine [316];
(R)—N-(3,3-difluoro-1-(oxetan-3-yl)piperidin-4-yl)-5-(1-(2,2-difluoroethyl)-4-fluoro-1H-benzo[d]imidazol-6-yl)-6-fluoro-4-methoxypyrrolo[2,1-f][1,2,4]triazin-2-amine [317];
(R)—N-(3,3-difluoro-1-(oxetan-3-yl-3-d)piperidin-4-yl)-5-(1-(2,2-difluoroethyl)-4-fluoro-1H-benzo[d]imidazol-6-yl)-6-fluoro-4-methoxypyrrolo[2,1-f][1,2,4]triazin-2-amine [318];
5-(1-(2,2-difluoroethyl)-4-fluoro-1H-benzo[d]imidazol-6-yl)-6-fluoro-4-methoxy-N-(2-oxaspiro[3.5]nonan-7-yl)pyrrolo[2,1-f][1,2,4]triazin-2-amine [319];
(1s,4s)-4-((5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-yl)amino)-1-methylcyclohexan-1-ol [320];
(1r,4r)-4-((5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-yl)amino)-1-methylcyclohexan-1-ol [321];
(R)—N-(4,4-difluoro-1-methylpyrrolidin-3-yl)-5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-amine [322];
(R)-1-(4-((5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-yl)amino)-3,3-difluoropyrrolidin-1-yl)ethan-1-one [323];
(R)—N-(4,4-difluoro-1-(oxetan-3-yl)pyrrolidin-3-yl)-5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-amine [324];
5-(1-(2,2-difluoroethyl)-4-fluoro-1H-benzo[d]imidazol-6-yl)-6-fluoro-4-(methoxy-d₃)—N-(1-(oxetan-3-yl)piperidin-4-yl)pyrrolo[2,1-f][1,2,4]triazin-2-amine [325];
5-(1-(2,2-difluoroethyl)-4-fluoro-1H-benzo[d]imidazol-6-yl)-6-fluoro-N-((3S,4R)-3-fluoro-1-methylpiperidin-4-yl)-4-(methoxy-d₃)pyrrolo[2,1-f][1,2,4]triazin-2-amine [326];
5-(1-(2,2-difluoroethyl)-4-fluoro-1H-benzo[d]imidazol-6-yl)-6-fluoro-N-((3R,4S)-3-fluoro-1-methylpiperidin-4-yl)-4-(methoxy-d₃)pyrrolo[2,1-f][1,2,4]triazin-2-amine [327];
1-((3R,4S)-4-((5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-yl)amino)-3-fluoropiperidin-1-yl)ethan-1-one [328];
1-((3R,4S)-4-((5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-yl)amino)-3-fluoropiperidin-1-yl)ethan-1-one-2,2,2-d₃ [329];
1-((3R,4S)-4-((5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-yl)amino)-3-fluoropiperidin-1-yl)-2-hydroxyethan-1-one [330];
5-(1-(2,2-difluoroethyl)-4-fluoro-1H-benzo[d]imidazol-6-yl)-6-fluoro-N-((3S,4R)-3-fluoro-1-(oxetan-3-yl)piperidin-4-yl)-4-(methoxy-d₃)pyrrolo[2,1-f][1,2,4]triazin-2-amine [331];
5-(1-(2,2-difluoroethyl)-4-fluoro-1H-benzo[d]imidazol-6-yl)-6-fluoro-N-((3R,4R)-3-fluoro-1-(oxetan-3-yl)piperidin-4-yl)-4-(methoxy-d₃)pyrrolo[2,1-f][1,2,4]triazin-2-amine [332];
5-(1-(2,2-difluoroethyl)-4-fluoro-1H-benzo[d]imidazol-6-yl)-6-fluoro-N-((3S,4S)-3-fluoro-1-(oxetan-3-yl)piperidin-4-yl)-4-(methoxy-d₃)pyrrolo[2,1-f][1,2,4]triazin-2-amine [333];
5-(1-(2,2-difluoroethyl)-4-fluoro-1H-benzo[d]imidazol-6-yl)-6-fluoro-N-((3R,4S)-3-fluoro-1-(oxetan-3-yl)piperidin-4-yl)-4-(methoxy-d₃)pyrrolo[2,1-f][1,2,4]triazin-2-amine [334];
(R)—N-(3,3-difluoro-1-methylpiperidin-4-yl)-5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-amine [335];
(R)—N-(3,3-difluoro-1-(methyl-d₃)piperidin-4-yl)-5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-amine [336];
(R)-1-(4-((5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-yl)amino)-3,3-difluoropiperidin-1-yl)ethan-1-one [337];
(R)-1-(4-((5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-yl)amino)-3,3-difluoropiperidin-1-yl)ethan-1-one-2,2,2-d_{3 [338];}
(R)-1-(4-((5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-yl)amino)-3,3-difluoropiperidin-1-yl)-2-hydroxyethan-1-one [339];
(R)—N-(3,3-difluoro-1-(methylsulfonyl)piperidin-4-yl)-5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-amine [340];
(R)—N-(3,3-difluoro-1-(oxetan-3-yl)piperidin-4-yl)-5-(1-(2,2-difluoroethyl)-4-fluoro-1H-benzo[d]imidazol-6-yl)-6-fluoro-4-(methoxy-d₃)pyrrolo[2,1-f][1,2,4]triazin-2-amine [341];
5-(1-(2,2-difluoroethyl)-4-fluoro-1H-benzo[d]imidazol-6-yl)-6-fluoro-4-(methoxy-d₃)—N-(2-oxaspiro[3.5]nonan-7-yl)pyrrolo[2,1-f][1,2,4]triazin-2-amine [342];
(R)-1-(4-((5-(benzo[d]thiazol-6-yl)-4-methoxypyrrolo[2,1-f][1,2,4]triazin-2-yl)amino)-3,3-difluoropiperidin-1-yl)ethan-1-one [343];
3-((5-(1-(2-fluoroethyl)-2-methyl-1H-benzo[d]imidazol-6-yl)-4-methoxypyrrolo[2,1-f][1,2,4]triazin-2-yl)amino)-2,2-dimethylpropanenitrile [344];
(1s,4s)-4-((5-(1-(2-fluoroethyl)-2-methyl-1H-benzo[d]imidazol-6-yl)-4-methoxypyrrolo[2,1-f][1,2,4]triazin-2-yl)amino)-1-methylcyclohexan-1-ol [345];
(1r,4r)-4-((5-(1-(2-fluoroethyl)-2-methyl-1H-benzo[d]imidazol-6-yl)-4-methoxypyrrolo[2,1-f][1,2,4]triazin-2-yl)amino)-1-methylcyclohexan-1-ol [346];
(R)—N-(4,4-difluoro-1-methylpyrrolidin-3-yl)-5-(1-(2-fluoroethyl)-2-methyl-1H-benzo[d]imidazol-6-yl)-4-methoxypyrrolo[2,1-f][1,2,4]triazin-2-amine [347];
(R)-1-(3,3-difluoro-4-((5-(1-(2-fluoroethyl)-2-methyl-1H-benzo[d]imidazol-6-yl)-4-methoxypyrrolo[2,1-f][1,2,4]triazin-2-yl)amino)pyrrolidin-1-yl)ethan-1-one [348];
(R)—N-(4,4-difluoro-1-(oxetan-3-yl)pyrrolidin-3-yl)-5-(1-(2-fluoroethyl)-2-methyl-1H-benzo[d]imidazol-6-yl)-4-methoxypyrrolo[2,1-f][1,2,4]triazin-2-amine [349];
5-(1-(2-fluoroethyl)-2-methyl-1H-benzo[d]imidazol-6-yl)-4-methoxy-N-(1-(oxetan-3-yl)piperidin-4-yl)pyrrolo[2,1-f][1,2,4]triazin-2-amine [350];
N-((3S,4R)-3-fluoro-1-methylpiperidin-4-yl)-5-(1-(2-fluoroethyl)-2-methyl-1H-benzo[d]imidazol-6-yl)-4-methoxypyrrolo[2,1-f][1,2,4]triazin-2-amine [351];
N-((3R,4S)-3-fluoro-1-methylpiperidin-4-yl)-5-(1-(2-fluoroethyl)-2-methyl-1H-benzo[d]imidazol-6-yl)-4-methoxypyrrolo[2,1-f][1,2,4]triazin-2-amine [352];
1-((3R,4S)-3-fluoro-4-((5-(1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one [353];
1-((3R,4S)-3-fluoro-4-((5-(1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one-2,2,2-d₃ [354];
1-((3R,4S)-3-fluoro-4-((5-(1-(2-fluoroethyl)-2-methyl-1H-benzo[d]imidazol-6-yl)-4-methoxypyrrolo[2,1-f][1,2,4]triazin-2-yl)amino)piperidin-1-yl)-2-hydroxyethan-1-one [355];
N-((3S,4R)-3-fluoro-1-(oxetan-3-yl)piperidin-4-yl)-5-(1-(2-fluoroethyl)-2-methyl-1H-benzo[d]imidazol-6-yl)-4-methoxypyrrolo[2,1-f][1,2,4]triazin-2-amine [356];
N-((3R,4R)-3-fluoro-1-(oxetan-3-yl)piperidin-4-yl)-5-(1-(2-fluoroethyl)-2-methyl-1H-benzo[d]imidazol-6-yl)-4-methoxypyrrolo[2,1-f][1,2,4]triazin-2-amine [357];
N-((3S,4S)-3-fluoro-1-(oxetan-3-yl)piperidin-4-yl)-5-(1-(2-fluoroethyl)-2-methyl-1H-benzo[d]imidazol-6-yl)-4-methoxypyrrolo[2,1-f][1,2,4]triazin-2-amine [358];
N-((3R,4S)-3-fluoro-1-(oxetan-3-yl)piperidin-4-yl)-5-(1-(2-fluoroethyl)-2-methyl-1H-benzo[d]imidazol-6-yl)-4-methoxypyrrolo[2,1-f][1,2,4]triazin-2-amine [359];
(R)—N-(3,3-difluoro-1-methylpiperidin-4-yl)-5-(1-(2-fluoroethyl)-2-methyl-1H-benzo[d]imidazol-6-yl)-4-methoxypyrrolo[2,1-f][1,2,4]triazin-2-amine [360];
(R)—N-(3,3-difluoro-1-(methyl-d₃)piperidin-4-yl)-5-(1-(2-fluoroethyl)-2-methyl-1H-benzo[d]imidazol-6-yl)-4-methoxypyrrolo[2,1-f][1,2,4]triazin-2-amine [361];
(R)-1-(3,3-difluoro-4-((5-(1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one [362];
(R)-1-(3,3-difluoro-4-((5-(1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one-2,2,2-d₃ [363];
(R)-1-(3,3-difluoro-4-((5-(1-(2-fluoroethyl)-2-methyl-1H-benzo[d]imidazol-6-yl)-4-methoxypyrrolo[2,1-f][1,2,4]triazin-2-yl)amino)piperidin-1-yl)-2-hydroxyethan-1-one [364];
(R)—N-(3,3-difluoro-1-(methylsulfonyl)piperidin-4-yl)-5-(1-(2-fluoroethyl)-2-methyl-1H-benzo[d]imidazol-6-yl)-4-methoxypyrrolo[2,1-f][1,2,4]triazin-2-amine [365];
(R)—N-(3,3-difluoro-1-(oxetan-3-yl)piperidin-4-yl)-5-(1-(2-fluoroethyl)-2-methyl-1H-benzo[d]imidazol-6-yl)-4-methoxypyrrolo[2,1-f][1,2,4]triazin-2-amine [366];
5-(1-(2-fluoroethyl)-2-methyl-1H-benzo[d]imidazol-6-yl)-4-methoxy-N-(2-oxaspiro[3.5]nonan-7-yl)pyrrolo[2,1-f][1,2,4]triazin-2-amine [367];
3-((6-fluoro-5-(1-(2-fluoroethyl)-2-methyl-1H-benzo[d]imidazol-6-yl)-4-methoxypyrrolo[2,1-f][1,2,4]triazin-2-yl)amino)-2,2-dimethylpropanenitrile [368];
(1s,4s)-4-((6-fluoro-5-(1-(2-fluoroethyl)-2-methyl-1H-benzo[d]imidazol-6-yl)-4-methoxypyrrolo[2,1-f][1,2,4]triazin-2-yl)amino)-1-methylcyclohexan-1-ol [369];
(1r,4r)-4-((6-fluoro-5-(1-(2-fluoroethyl)-2-methyl-1H-benzo[d]imidazol-6-yl)-4-methoxypyrrolo[2,1-f][1,2,4]triazin-2-yl)amino)-1-methylcyclohexan-1-ol [370];
(R)—N-(4,4-difluoro-1-methylpyrrolidin-3-yl)-6-fluoro-5-(1-(2-fluoroethyl)-2-methyl-1H-benzo[d]imidazol-6-yl)-4-methoxypyrrolo[2,1-f][1,2,4]triazin-2-amine [371];
(R)-1-(3,3-difluoro-4-((6-fluoro-5-(1-(2-fluoroethyl)-2-methyl-1H-benzo[d]imidazol-6-yl)-4-methoxypyrrolo[2,1-f][1,2,4]triazin-2-yl)amino)pyrrolidin-1-yl)ethan-1-one [372];
(R)—N-(4,4-difluoro-1-(oxetan-3-yl)pyrrolidin-3-yl)-6-fluoro-5-(1-(2-fluoroethyl)-2-methyl-1H-benzo[d]imidazol-6-yl)-4-methoxypyrrolo[2,1-f][1,2,4]triazin-2-amine [373];
6-fluoro-5-(1-(2-fluoroethyl)-2-methyl-1H-benzo[d]imidazol-6-yl)-4-methoxy-N-(1-(oxetan-3-yl)piperidin-4-yl)pyrrolo[2,1-f][1,2,4]triazin-2-amine [374];
6-fluoro-N-((3S,4R)-3-fluoro-1-methylpiperidin-4-yl)-5-(1-(2-fluoroethyl)-2-methyl-1H-benzo[d]imidazol-6-yl)-4-methoxypyrrolo[2,1-f][1,2,4]triazin-2-amine [375];
6-fluoro-N-((3R,4S)-3-fluoro-1-methylpiperidin-4-yl)-5-(1-(2-fluoroethyl)-2-methyl-1H-benzo[d]imidazol-6-yl)-4-methoxypyrrolo[2,1-f][1,2,4]triazin-2-amine [376];
1-((3R,4S)-3-fluoro-4-((6-fluoro-5-(1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one [377];
1-((3R,4S)-3-fluoro-4-((6-fluoro-5-(1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one-2,2,2-d₃ [378];
1-((3R,4S)-3-fluoro-4-((6-fluoro-5-(1-(2-fluoroethyl)-2-methyl-1H-benzo[d]imidazol-6-yl)-4-methoxypyrrolo[2,1-f][1,2,4]triazin-2-yl)amino)piperidin-1-yl)-2-hydroxyethan-1-one [379];
6-fluoro-N-((3S,4R)-3-fluoro-1-(oxetan-3-yl)piperidin-4-yl)-5-(1-(2-fluoroethyl)-2-methyl-1H-benzo[d]imidazol-6-yl)-4-methoxypyrrolo[2,1-f][1,2,4]triazin-2-amine [380];
6-fluoro-N-((3R,4R)-3-fluoro-1-(oxetan-3-yl)piperidin-4-yl)-5-(1-(2-fluoroethyl)-2-methyl-1H-benzo[d]imidazol-6-yl)-4-methoxypyrrolo[2,1-f][1,2,4]triazin-2-amine [381];
6-fluoro-N-((3S,4S)-3-fluoro-1-(oxetan-3-yl)piperidin-4-yl)-5-(1-(2-fluoroethyl)-2-methyl-1H-benzo[d]imidazol-6-yl)-4-methoxypyrrolo[2,1-f][1,2,4]triazin-2-amine [382];
6-fluoro-N-((3R,4S)-3-fluoro-1-(oxetan-3-yl)piperidin-4-yl)-5-(1-(2-fluoroethyl)-2-methyl-1H-benzo[d]imidazol-6-yl)-4-methoxypyrrolo[2,1-f][1,2,4]triazin-2-amine [383];
(R)—N-(3,3-difluoro-1-methylpiperidin-4-yl)-6-fluoro-5-(1-(2-fluoroethyl)-2-methyl-1H-benzo[d]imidazol-6-yl)-4-methoxypyrrolo[2,1-f][1,2,4]triazin-2-amine [384];
(R)—N-(3,3-difluoro-1-(methyl-d₃)piperidin-4-yl)-6-fluoro-5-(1-(2-fluoroethyl)-2-methyl-1H-benzo[d]imidazol-6-yl)-4-methoxypyrrolo[2,1-f][1,2,4]triazin-2-amine [385];
(R)-1-(3,3-difluoro-4-((6-fluoro-5-(1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one [386];
(R)-1-(3,3-difluoro-4-((6-fluoro-5-(1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one-2,2,2-d₃ [387];
(R)-1-(3,3-difluoro-4-((6-fluoro-5-(1-(2-fluoroethyl)-2-methyl-1H-benzo[d]imidazol-6-yl)-4-methoxypyrrolo[2,1-f][1,2,4]triazin-2-yl)amino)piperidin-1-yl)-2-hydroxyethan-1-one [388];
(R)—N-(3,3-difluoro-1-(methylsulfonyl)piperidin-4-yl)-6-fluoro-5-(1-(2-fluoroethyl)-2-methyl-1H-benzo[d]imidazol-6-yl)-4-methoxypyrrolo[2,1-f][1,2,4]triazin-2-amine [389];
(R)—N-(3,3-difluoro-1-(oxetan-3-yl)piperidin-4-yl)-6-fluoro-5-(1-(2-fluoroethyl)-2-methyl-1H-benzo[d]imidazol-6-yl)-4-methoxypyrrolo[2,1-f][1,2,4]triazin-2-amine [390];
6-fluoro-5-(1-(2-fluoroethyl)-2-methyl-1H-benzo[d]imidazol-6-yl)-4-methoxy-N-(2-oxaspiro[3.5]nonan-7-yl)pyrrolo[2,1-f][1,2,4]triazin-2-amine [391];
3-((5-(4-fluoro-1-(2-fluoroethyl)-2-methyl-1H-benzo[d]imidazol-6-yl)-4-methoxypyrrolo[2,1-f][1,2,4]triazin-2-yl)amino)-2,2-dimethylpropanenitrile [392];
(1s,4s)-4-((5-(4-fluoro-1-(2-fluoroethyl)-2-methyl-1H-benzo[d]imidazol-6-yl)-4-methoxypyrrolo[2,1-f][1,2,4]triazin-2-yl)amino)-1-methylcyclohexan-1-ol [393];
(1r,4r)-4-((5-(4-fluoro-1-(2-fluoroethyl)-2-methyl-1H-benzo[d]imidazol-6-yl)-4-methoxypyrrolo[2,1-f][1,2,4]triazin-2-yl)amino)-1-methylcyclohexan-1-ol [394];
(R)—N-(4,4-difluoro-1-methylpyrrolidin-3-yl)-5-(4-fluoro-1-(2-fluoroethyl)-2-methyl-1H-benzo[d]imidazol-6-yl)-4-methoxypyrrolo[2,1-f][1,2,4]triazin-2-amine [395];
(R)-1-(3,3-difluoro-4-((5-(4-fluoro-1-(2-fluoroethyl)-2-methyl-1H-benzo[d]imidazol-6-yl)-4-methoxypyrrolo[2,1-f][1,2,4]triazin-2-yl)amino)pyrrolidin-1-yl)ethan-1-one [396];
(R)—N-(4,4-difluoro-1-(oxetan-3-yl)pyrrolidin-3-yl)-5-(4-fluoro-1-(2-fluoroethyl)-2-methyl-1H-benzo[d]imidazol-6-yl)-4-methoxypyrrolo[2,1-f][1,2,4]triazin-2-amine [397];
5-(4-fluoro-1-(2-fluoroethyl)-2-methyl-1H-benzo[d]imidazol-6-yl)-4-methoxy-N-(1-(oxetan-3-yl)piperidin-4-yl)pyrrolo[2,1-f][1,2,4]triazin-2-amine [398];
5-(4-fluoro-1-(2-fluoroethyl)-2-methyl-1H-benzo[d]imidazol-6-yl)-N-((3S,4R)-3-fluoro-1-methylpiperidin-4-yl)-4-methoxypyrrolo[2,1-f][1,2,4]triazin-2-amine [399]; and
5-(4-fluoro-1-(2-fluoroethyl)-2-methyl-1H-benzo[d]imidazol-6-yl)-N-((3R,4S)-3-fluoro-1-methylpiperidin-4-yl)-4-methoxypyrrolo[2,1-f][1,2,4]triazin-2-amine [400]; or a pharmaceutically acceptable salt thereof.

21. The compound of claim 1, wherein the compound of Formula I is selected from the group consisting of
1-((3R,4S)-3-fluoro-4-((5-(4-fluoro-1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one [401];
1-((3R,4S)-3-fluoro-4-((5-(4-fluoro-1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one-2,2,2-d₃ [402];
1-((3R,4S)-3-fluoro-4-((5-(4-fluoro-1-(2-fluoroethyl)-2-methyl-1H-benzo[d]imidazol-6-yl)-4-methoxypyrrolo[2,1-f][1,2,4]triazin-2-yl)amino)piperidin-1-yl)-2-hydroxyethan-1-one [403];
5-(4-fluoro-1-(2-fluoroethyl)-2-methyl-1H-benzo[d]imidazol-6-yl)-N-((3S,4R)-3-fluoro-1-(oxetan-3-yl)piperidin-4-yl)-4-methoxypyrrolo[2,1-f][1,2,4]triazin-2-amine [404];
5-(4-fluoro-1-(2-fluoroethyl)-2-methyl-1H-benzo[d]imidazol-6-yl)-N-((3R,4R)-3-fluoro-1-(oxetan-3-yl)piperidin-4-yl)-4-methoxypyrrolo[2,1-f][1,2,4]triazin-2-amine [405];
5-(4-fluoro-1-(2-fluoroethyl)-2-methyl-1H-benzo[d]imidazol-6-yl)-N-((3S,4S)-3-fluoro-1-(oxetan-3-yl)piperidin-4-yl)-4-methoxypyrrolo[2,1-f][1,2,4]triazin-2-amine [406];
5-(4-fluoro-1-(2-fluoroethyl)-2-methyl-1H-benzo[d]imidazol-6-yl)-N-((3R,4S)-3-fluoro-1-(oxetan-3-yl)piperidin-4-yl)-4-methoxypyrrolo[2,1-f][1,2,4]triazin-2-amine [407];
(R)—N-(3,3-difluoro-1-methylpiperidin-4-yl)-5-(4-fluoro-1-(2-fluoroethyl)-2-methyl-1H-benzo[d]imidazol-6-yl)-4-methoxypyrrolo[2,1-f][1,2,4]triazin-2-amine [408];
(R)—N-(3,3-difluoro-1-(methyl-d₃)piperidin-4-yl)-5-(4-fluoro-1-(2-fluoroethyl)-2-methyl-1H-benzo[d]imidazol-6-yl)-4-methoxypyrrolo[2,1-f][1,2,4]triazin-2-amine [409];
(R)-1-(3,3-difluoro-4-((5-(4-fluoro-1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one [410];
(R)-1-(3,3-difluoro-4-((5-(4-fluoro-1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one-2,2,2-d₃ [411];
(R)-1-(3,3-difluoro-4-((5-(4-fluoro-1-(2-fluoroethyl)-2-methyl-1H-benzo[d]imidazol-6-yl)-4-methoxypyrrolo[2,1-f][1,2,4]triazin-2-yl)amino)piperidin-1-yl)-2-hydroxyethan-1-one [412];
(R)—N-(3,3-difluoro-1-(methylsulfonyl)piperidin-4-yl)-5-(4-fluoro-1-(2-fluoroethyl)-2-methyl-1H-benzo[d]imidazol-6-yl)-4-methoxypyrrolo[2,1-f][1,2,4]triazin-2-amine [413];
(R)—N-(3,3-difluoro-1-(oxetan-3-yl)piperidin-4-yl)-5-(4-fluoro-1-(2-fluoroethyl)-2-methyl-1H-benzo[d]imidazol-6-yl)-4-methoxypyrrolo[2,1-f][1,2,4]triazin-2-amine [414];
5-(4-fluoro-1-(2-fluoroethyl)-2-methyl-1H-benzo[d]imidazol-6-yl)-4-methoxy-N-(2-oxaspiro[3.5]nonan-7-yl)pyrrolo[2,1-f][1,2,4]triazin-2-amine [415];
3-((6-fluoro-5-(4-fluoro-1-(2-fluoroethyl)-2-methyl-1H-benzo[d]imidazol-6-yl)-4-methoxypyrrolo[2,1-f][1,2,4]triazin-2-yl)amino)-2,2-dimethylpropanenitrile [416];
(1s,4s)-4-((6-fluoro-5-(4-fluoro-1-(2-fluoroethyl)-2-methyl-1H-benzo[d]imidazol-6-yl)-4-methoxypyrrolo[2,1-f][1,2,4]triazin-2-yl)amino)-1-methylcyclohexan-1-ol [417];
(1r,4r)-4-((6-fluoro-5-(4-fluoro-1-(2-fluoroethyl)-2-methyl-1H-benzo[d]imidazol-6-yl)-4-methoxypyrrolo[2,1-f][1,2,4]triazin-2-yl)amino)-1-methylcyclohexan-1-ol [418];
(R)—N-(4,4-difluoro-1-methylpyrrolidin-3-yl)-6-fluoro-5-(4-fluoro-1-(2-fluoroethyl)-2-methyl-1H-benzo[d]imidazol-6-yl)-4-methoxypyrrolo[2,1-f][1,2,4]triazin-2-amine [419];
(R)-1-(3,3-difluoro-4-((6-fluoro-5-(4-fluoro-1-(2-fluoroethyl)-2-methyl-1H-benzo[d]imidazol-6-yl)-4-methoxypyrrolo[2,1-f][1,2,4]triazin-2-yl)amino)pyrrolidin-1-yl)ethan-1-one [420];
(R)—N-(4,4-difluoro-1-(oxetan-3-yl)pyrrolidin-3-yl)-6-fluoro-5-(4-fluoro-1-(2-fluoroethyl)-2-methyl-1H-benzo[d]imidazol-6-yl)-4-methoxypyrrolo[2,1-f][1,2,4]triazin-2-amine [421];
6-fluoro-5-(4-fluoro-1-(2-fluoroethyl)-2-methyl-1H-benzo[d]imidazol-6-yl)-4-methoxy-N-(1-(oxetan-3-yl)piperidin-4-yl)pyrrolo[2,1-f][1,2,4]triazin-2-amine [422];
6-fluoro-5-(4-fluoro-1-(2-fluoroethyl)-2-methyl-1H-benzo[d]imidazol-6-yl)-N-((3S,4R)-3-fluoro-1-methylpiperidin-4-yl)-4-methoxypyrrolo[2,1-f][1,2,4]triazin-2-amine [423];
6-fluoro-5-(4-fluoro-1-(2-fluoroethyl)-2-methyl-1H-benzo[d]imidazol-6-yl)-N-((3R,4S)-3-fluoro-1-methylpiperidin-4-yl)-4-methoxypyrrolo[2,1-f][1,2,4]triazin-2-amine [424];
1-((3R,4S)-3-fluoro-4-((6-fluoro-5-(4-fluoro-1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one [425];
1-((3R,4S)-3-fluoro-4-((6-fluoro-5-(4-fluoro-1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one-2,2,2-d₃ [426];
1-((3R,4S)-3-fluoro-4-((6-fluoro-5-(4-fluoro-1-(2-fluoroethyl)-2-methyl-1H-benzo[d]imidazol-6-yl)-4-methoxypyrrolo[2,1-f][1,2,4]triazin-2-yl)amino)piperidin-1-yl)-2-hydroxyethan-1-one [427];
6-fluoro-5-(4-fluoro-1-(2-fluoroethyl)-2-methyl-1H-benzo[d]imidazol-6-yl)-N-((3S,4R)-3-fluoro-1-(oxetan-3-yl)piperidin-4-yl)-4-methoxypyrrolo[2,1-f][1,2,4]triazin-2-amine [428];
6-fluoro-5-(4-fluoro-1-(2-fluoroethyl)-2-methyl-1H-benzo[d]imidazol-6-yl)-N-((3R,4R)-3-fluoro-1-(oxetan-3-yl)piperidin-4-yl)-4-methoxypyrrolo[2,1-f][1,2,4]triazin-2-amine [429];
6-fluoro-5-(4-fluoro-1-(2-fluoroethyl)-2-methyl-1H-benzo[d]imidazol-6-yl)-N-((3S,4S)-3-fluoro-1-(oxetan-3-yl)piperidin-4-yl)-4-methoxypyrrolo[2,1-f][1,2,4]triazin-2-amine [430];
6-fluoro-5-(4-fluoro-1-(2-fluoroethyl)-2-methyl-1H-benzo[d]imidazol-6-yl)-N-((3R,4S)-3-fluoro-1-(oxetan-3-yl)piperidin-4-yl)-4-methoxypyrrolo[2,1-f][1,2,4]triazin-2-amine [431];
(R)—N-(3,3-difluoro-1-methylpiperidin-4-yl)-6-fluoro-5-(4-fluoro-1-(2-fluoroethyl)-2-methyl-1H-benzo[d]imidazol-6-yl)-4-methoxypyrrolo[2,1-f][1,2,4]triazin-2-amine [432];
(R)—N-(3,3-difluoro-1-(methyl-d₃)piperidin-4-yl)-6-fluoro-5-(4-fluoro-1-(2-fluoroethyl)-2-methyl-1H-benzo[d]imidazol-6-yl)-4-methoxypyrrolo[2,1-f][1,2,4]triazin-2-amine [433];
(R)-1-(3,3-difluoro-4-((6-fluoro-5-(4-fluoro-1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one [434];
(R)-1-(3,3-difluoro-4-((6-fluoro-5-(4-fluoro-1-(2-fluoroethyl)-2-methyl-1H-benzo[d]imidazol-6-yl)-4-methoxypyrrolo[2,1-f][1,2,4]triazin-2-yl)amino)piperidin-1-yl)ethan-1-one-2,2,2-d₃ [435];
(R)-1-(3,3-difluoro-4-((6-fluoro-5-(4-fluoro-1-(2-fluoroethyl)-2-methyl-1H-benzo[d]imidazol-6-yl)-4-methoxypyrrolo[2,1-f][1,2,4]triazin-2-yl)amino)piperidin-1-yl)-2-hydroxyethan-1-one [436];
(R)—N-(3,3-difluoro-1-(methylsulfonyl)piperidin-4-yl)-6-fluoro-5-(4-fluoro-1-(2-fluoroethyl)-2-methyl-1H-benzo[d]imidazol-6-yl)-4-methoxypyrrolo[2,1-f][1,2,4]triazin-2-amine [437];
(R)—N-(3,3-difluoro-1-(oxetan-3-yl)piperidin-4-yl)-6-fluoro-5-(4-fluoro-1-(2-fluoroethyl)-2-methyl-1H-benzo[d]imidazol-6-yl)-4-methoxypyrrolo[2,1-f][1,2,4]triazin-2-amine [438];
6-fluoro-5-(4-fluoro-1-(2-fluoroethyl)-2-methyl-1H-benzo[d]imidazol-6-yl)-4-methoxy-N-(2-oxaspiro[3.5]nonan-7-yl)pyrrolo[2,1-f][1,2,4]triazin-2-amine [439];
5-(1-(2,2-difluoroethyl)-2-methyl-1H-benzo[d]imidazol-6-yl)-N-((3S,4R)-3-fluoro-1-(2-methoxyethyl)piperidin-4-yl)-4-methoxypyrrolo[2,1-f][1,2,4]triazin-2-amine [440];
5-(1-(2,2-difluoroethyl)-2-methyl-1H-benzo[d]imidazol-6-yl)-N-((3R,4R)-3-fluoro-1-(2-methoxyethyl)piperidin-4-yl)-4-methoxypyrrolo[2,1-f][1,2,4]triazin-2-amine [441];
5-(1-(2,2-difluoroethyl)-2-methyl-1H-benzo[d]imidazol-6-yl)-N-((3S,4S)-3-fluoro-1-(2-methoxyethyl)piperidin-4-yl)-4-methoxypyrrolo[2,1-f][1,2,4]triazin-2-amine [442];
5-(1-(2,2-difluoroethyl)-2-methyl-1H-benzo[d]imidazol-6-yl)-N-((3R,4S)-3-fluoro-1-(2-methoxyethyl)piperidin-4-yl)-4-methoxypyrrolo[2,1-f][1,2,4]triazin-2-amine [443];
1-((3R,4S)-4-((5-(1-(2,2-difluoroethyl)-2-methyl-1H-benzo[d]imidazol-6-yl)-4-methoxypyrrolo[2,1-f][1,2,4]triazin-2-yl)amino)-3-fluoropiperidin-1-yl)ethan-1-one [444];
1-((3R,4S)-4-((5-(1-(2,2-difluoroethyl)-2-methyl-1H-benzo[d]imidazol-6-yl)-4-methoxypyrrolo[2,1-f][1,2,4]triazin-2-yl)amino)-3-fluoropiperidin-1-yl)-2-hydroxyethan-1-one [445];
5-(1-(2,2-difluoroethyl)-2-methyl-1H-benzo[d]imidazol-6-yl)-N-((3S,4R)-3-fluoro-1-(oxetan-3-yl-3-d)piperidin-4-yl)-4-methoxypyrrolo[2,1-f][1,2,4]triazin-2-amine [446];
5-(1-(2,2-difluoroethyl)-2-methyl-1H-benzo[d]imidazol-6-yl)-N-((3R,4R)-3-fluoro-1-(oxetan-3-yl-3-d)piperidin-4-yl)-4-methoxypyrrolo[2,1-f][1,2,4]triazin-2-amine [447];
5-(1-(2,2-difluoroethyl)-2-methyl-1H-benzo[d]imidazol-6-yl)-N-((3S,4S)-3-fluoro-1-(oxetan-3-yl-3-d)piperidin-4-yl)-4-methoxypyrrolo[2,1-f][1,2,4]triazin-2-amine [448];
5-(1-(2,2-difluoroethyl)-2-methyl-1H-benzo[d]imidazol-6-yl)-N-((3R,4S)-3-fluoro-1-(oxetan-3-yl-3-d)piperidin-4-yl)-4-methoxypyrrolo[2,1-f][1,2,4]triazin-2-amine [449];
(R)—N-(3,3-difluoro-1-(2-methoxyethyl)piperidin-4-yl)-5-(1-(2,2-difluoroethyl)-2-methyl-1H-benzo[d]imidazol-6-yl)-4-methoxypyrrolo[2,1-f][1,2,4]triazin-2-amine [450];
(R)-1-(4-((5-(1-(2,2-difluoroethyl)-2-methyl-1H-benzo[d]imidazol-6-yl)-4-methoxypyrrolo[2,1-f][1,2,4]triazin-2-yl)amino)-3,3-difluoropiperidin-1-yl)ethan-1-one [451];
(R)-1-(4-((5-(1-(2,2-difluoroethyl)-2-methyl-1H-benzo[d]imidazol-6-yl)-4-methoxypyrrolo[2,1-f][1,2,4]triazin-2-yl)amino)-3,3-difluoropiperidin-1-yl)-2-hydroxyethan-1-one [452];
(R)—N-(3,3-difluoro-1-(methylsulfonyl)piperidin-4-yl)-5-(1-(2,2-difluoroethyl)-2-methyl-1H-benzo[d]imidazol-6-yl)-4-methoxypyrrolo[2,1-f][1,2,4]triazin-2-amine [453];
(R)—N-(3,3-difluoro-1-(oxetan-3-yl-3-d)piperidin-4-yl)-5-(1-(2,2-difluoroethyl)-2-methyl-1H-benzo[d]imidazol-6-yl)-4-methoxypyrrolo[2,1-f][1,2,4]triazin-2-amine [454];
3-((5-(1-(2,2-difluoroethyl)-2-methyl-1H-benzo[d]imidazol-6-yl)-6-fluoro-4-methoxypyrrolo[2,1-f][1,2,4]triazin-2-yl)amino)-2,2-dimethylpropanenitrile [455];
(1S,4S)-4-((5-(1-(2,2-difluoroethyl)-2-methyl-1H-benzo[d]imidazol-6-yl)-6-fluoro-4-methoxypyrrolo[2,1-f][1,2,4]triazin-2-yl)amino)-1-methylcyclohexan-1-ol [456];
(1R,4R)-4-((5-(1-(2,2-difluoroethyl)-2-methyl-1H-benzo[d]imidazol-6-yl)-6-fluoro-4-methoxypyrrolo[2,1-f][1,2,4]triazin-2-yl)amino)-1-methylcyclohexan-1-ol [457];
(R)—N-(4,4-difluoro-1-methylpyrrolidin-3-yl)-5-(1-(2,2-difluoroethyl)-2-methyl-1H-benzo[d]imidazol-6-yl)-6-fluoro-4-methoxypyrrolo[2,1-f][1,2,4]triazin-2-amine [458];
(R)-1-(4-((5-(1-(2,2-difluoroethyl)-2-methyl-1H-benzo[d]imidazol-6-yl)-6-fluoro-4-methoxypyrrolo[2,1-f][1,2,4]triazin-2-yl)amino)-3,3-difluoropyrrolidin-1-yl)ethan-1-one [459];
(R)—N-(4,4-difluoro-1-(oxetan-3-yl)pyrrolidin-3-yl)-5-(1-(2,2-difluoroethyl)-2-methyl-1H-benzo[d]imidazol-6-yl)-6-fluoro-4-methoxypyrrolo[2,1-f][1,2,4]triazin-2-amine [460];
5-(1-(2,2-difluoroethyl)-2-methyl-1H-benzo[d]imidazol-6-yl)-6-fluoro-4-methoxy-N-(1-(oxetan-3-yl)piperidin-4-yl)pyrrolo[2,1-f][1,2,4]triazin-2-amine [461];
5-(1-(2,2-difluoroethyl)-2-methyl-1H-benzo[d]imidazol-6-yl)-6-fluoro-N-((3S,4R)-3-fluoro-1-methylpiperidin-4-yl)-4-methoxypyrrolo[2,1-f][1,2,4]triazin-2-amine [462];
5-(1-(2,2-difluoroethyl)-2-methyl-1H-benzo[d]imidazol-6-yl)-6-fluoro-N-((3R,4S)-3-fluoro-1-methylpiperidin-4-yl)-4-methoxypyrrolo[2,1-f][1,2,4]triazin-2-amine [463];
5-(1-(2,2-difluoroethyl)-2-methyl-1H-benzo[d]imidazol-6-yl)-6-fluoro-N-((3S,4R)-3-fluoro-1-(2-methoxyethyl)piperidin-4-yl)-4-methoxypyrrolo[2,1-f][1,2,4]triazin-2-amine [464];
5-(1-(2,2-difluoroethyl)-2-methyl-1H-benzo[d]imidazol-6-yl)-6-fluoro-N-((3R,4R)-3-fluoro-1-(2-methoxyethyl)piperidin-4-yl)-4-methoxypyrrolo[2,1-f][1,2,4]triazin-2-amine [465];
5-(1-(2,2-difluoroethyl)-2-methyl-1H-benzo[d]imidazol-6-yl)-6-fluoro-N-((3S,4S)-3-fluoro-1-(2-methoxyethyl)piperidin-4-yl)-4-methoxypyrrolo[2,1-f][1,2,4]triazin-2-amine [466];
5-(1-(2,2-difluoroethyl)-2-methyl-1H-benzo[d]imidazol-6-yl)-6-fluoro-N-((3R,4S)-3-fluoro-1-(2-methoxyethyl)piperidin-4-yl)-4-methoxypyrrolo[2,1-f][1,2,4]triazin-2-amine [467];
1-((3R,4S)-4-((5-(1-(2,2-difluoroethyl)-2-methyl-1H-benzo[d]imidazol-6-yl)-6-fluoro-4-methoxypyrrolo[2,1-f][1,2,4]triazin-2-yl)amino)-3-fluoropiperidin-1-yl)ethan-1-one [468];
1-((3R,4S)-4-((5-(1-(2,2-difluoroethyl)-2-methyl-1H-benzo[d]imidazol-6-yl)-6-fluoro-4-methoxypyrrolo[2,1-f][1,2,4]triazin-2-yl)amino)-3-fluoropiperidin-1-yl)ethan-1-one-2,2,2-d₃ [469];
1-((3R,4S)-4-((5-(1-(2,2-difluoroethyl)-2-methyl-1H-benzo[d]imidazol-6-yl)-6-fluoro-4-methoxypyrrolo[2,1-f][1,2,4]triazin-2-yl)amino)-3-fluoropiperidin-1-yl)-2-hydroxyethan-1-one [470];
5-(1-(2,2-difluoroethyl)-2-methyl-1H-benzo[d]imidazol-6-yl)-6-fluoro-N-((3S,4R)-3-fluoro-1-(oxetan-3-yl)piperidin-4-yl)-4-methoxypyrrolo[2,1-f][1,2,4]triazin-2-amine [471];
5-(1-(2,2-difluoroethyl)-2-methyl-1H-benzo[d]imidazol-6-yl)-6-fluoro-N-((3R,4R)-3-fluoro-1-(oxetan-3-yl)piperidin-4-yl)-4-methoxypyrrolo[2,1-f][1,2,4]triazin-2-amine [472];
5-(1-(2,2-difluoroethyl)-2-methyl-1H-benzo[d]imidazol-6-yl)-6-fluoro-N-((3S,4S)-3-fluoro-1-(oxetan-3-yl)piperidin-4-yl)-4-methoxypyrrolo[2,1-f][1,2,4]triazin-2-amine [473];
5-(1-(2,2-difluoroethyl)-2-methyl-1H-benzo[d]imidazol-6-yl)-6-fluoro-N-((3R,4S)-3-fluoro-1-(oxetan-3-yl)piperidin-4-yl)-4-methoxypyrrolo[2,1-f][1,2,4]triazin-2-amine [474];
5-(1-(2,2-difluoroethyl)-2-methyl-1H-benzo[d]imidazol-6-yl)-6-fluoro-N-((3S,4R)-3-fluoro-1-(oxetan-3-yl-3-d)piperidin-4-yl)-4-methoxypyrrolo[2,1-f][1,2,4]triazin-2-amine [475];
5-(1-(2,2-difluoroethyl)-2-methyl-1H-benzo[d]imidazol-6-yl)-6-fluoro-N-((3R,4R)-3-fluoro-1-(oxetan-3-yl-3-d)piperidin-4-yl)-4-methoxypyrrolo[2,1-f][1,2,4]triazin-2-amine [476];
5-(1-(2,2-difluoroethyl)-2-methyl-1H-benzo[d]imidazol-6-yl)-6-fluoro-N-((3S,4S)-3-fluoro-1-(oxetan-3-yl-3-d)piperidin-4-yl)-4-methoxypyrrolo[2,1-f][1,2,4]triazin-2-amine [477];
5-(1-(2,2-difluoroethyl)-2-methyl-1H-benzo[d]imidazol-6-yl)-6-fluoro-N-((3R,4S)-3-fluoro-1-(oxetan-3-yl-3-d)piperidin-4-yl)-4-methoxypyrrolo[2,1-f][1,2,4]triazin-2-amine [478];
(R)—N-(3,3-difluoro-1-methylpiperidin-4-yl)-5-(1-(2,2-difluoroethyl)-2-methyl-1H-benzo[d]imidazol-6-yl)-6-fluoro-4-methoxypyrrolo[2,1-f][1,2,4]triazin-2-amine [479];
(R)—N-(3,3-difluoro-1-(methyl-d₃)piperidin-4-yl)-5-(1-(2,2-difluoroethyl)-2-methyl-1H-benzo[d]imidazol-6-yl)-6-fluoro-4-methoxypyrrolo[2,1-f][1,2,4]triazin-2-amine [480];
(R)—N-(3,3-difluoro-1-(2-methoxyethyl)piperidin-4-yl)-5-(1-(2,2-difluoroethyl)-2-methyl-1H-benzo[d]imidazol-6-yl)-6-fluoro-4-methoxypyrrolo[2,1-f][1,2,4]triazin-2-amine [481];
(R)-1-(4-((5-(1-(2,2-difluoroethyl)-2-methyl-1H-benzo[d]imidazol-6-yl)-6-fluoro-4-methoxypyrrolo[2,1-f][1,2,4]triazin-2-yl)amino)-3,3-difluoropiperidin-1-yl)ethan-1-one [482];
(R)-1-(4-((5-(1-(2,2-difluoroethyl)-2-methyl-1H-benzo[d]imidazol-6-yl)-6-fluoro-4-methoxypyrrolo[2,1-f][1,2,4]triazin-2-yl)amino)-3,3-difluoropiperidin-1-yl)ethan-1-one-2,2,2-d₃ [483];
(R)-1-(4-((5-(1-(2,2-difluoroethyl)-2-methyl-1H-benzo[d]imidazol-6-yl)-6-fluoro-4-methoxypyrrolo[2,1-f][1,2,4]triazin-2-yl)amino)-3,3-difluoropiperidin-1-yl)-2-hydroxyethan-1-one [484];
(R)—N-(3,3-difluoro-1-(methylsulfonyl)piperidin-4-yl)-5-(1-(2,2-difluoroethyl)-2-methyl-1H-benzo[d]imidazol-6-yl)-6-fluoro-4-methoxypyrrolo[2,1-f][1,2,4]triazin-2-amine [485];
(R)—N-(3,3-difluoro-1-(oxetan-3-yl)piperidin-4-yl)-5-(1-(2,2-difluoroethyl)-2-methyl-1H-benzo[d]imidazol-6-yl)-6-fluoro-4-methoxypyrrolo[2,1-f][1,2,4]triazin-2-amine [486];
(R)—N-(3,3-difluoro-1-(oxetan-3-yl-3-d)piperidin-4-yl)-5-(1-(2,2-difluoroethyl)-2-methyl-1H-benzo[d]imidazol-6-yl)-6-fluoro-4-methoxypyrrolo[2,1-f][1,2,4]triazin-2-amine [487];
5-(1-(2,2-difluoroethyl)-2-methyl-1H-benzo[d]imidazol-6-yl)-6-fluoro-4-methoxy-N-(2-oxaspiro[3.5]nonan-7-yl)pyrrolo[2,1-f][1,2,4]triazin-2-amine [488];
(1S,4S)-4-((5-(1-(2,2-difluoroethyl)-2-methyl-1H-benzo[d]imidazol-6-yl)-6-fluoro-4-(methoxy-d₃)pyrrolo[2,1-f][1,2,4]triazin-2-yl)amino)-1-methylcyclohexan-1-ol [489];
(1R,4R)-4-((5-(1-(2,2-difluoroethyl)-2-methyl-1H-benzo[d]imidazol-6-yl)-6-fluoro-4-(methoxy-d₃)pyrrolo[2,1-f][1,2,4]triazin-2-yl)amino)-1-methylcyclohexan-1-ol [490];
(R)—N-(4,4-difluoro-1-methylpyrrolidin-3-yl)-5-(1-(2,2-difluoroethyl)-2-methyl-1H-benzo[d]imidazol-6-yl)-6-fluoro-4-(methoxy-d₃)pyrrolo[2,1-f][1,2,4]triazin-2-amine [491];
(R)-1-(4-((5-(1-(2,2-difluoroethyl)-2-methyl-1H-benzo[d]imidazol-6-yl)-6-fluoro-4-(methoxy-d₃)pyrrolo[2,1-f][1,2,4]triazin-2-yl)amino)-3,3-difluoropyrrolidin-1-yl)ethan-1-one [492];
(R)—N-(4,4-difluoro-1-(oxetan-3-yl)pyrrolidin-3-yl)-5-(1-(2,2-difluoroethyl)-2-methyl-1H-benzo[d]imidazol-6-yl)-6-fluoro-4-(methoxy-d₃)pyrrolo[2,1-f][1,2,4]triazin-2-amine [493];
5-(1-(2,2-difluoroethyl)-2-methyl-1H-benzo[d]imidazol-6-yl)-6-fluoro-4-(methoxy-d₃)—N-(1-(oxetan-3-yl)piperidin-4-yl)pyrrolo[2,1-f][1,2,4]triazin-2-amine [494];
5-(1-(2,2-difluoroethyl)-2-methyl-1H-benzo[d]imidazol-6-yl)-6-fluoro-N-((3S,4R)-3-fluoro-1-methylpiperidin-4-yl)-4-(methoxy-d₃)pyrrolo[2,1-f][1,2,4]triazin-2-amine [495];
5-(1-(2,2-difluoroethyl)-2-methyl-1H-benzo[d]imidazol-6-yl)-6-fluoro-N-((3R,4S)-3-fluoro-1-methylpiperidin-4-yl)-4-(methoxy-d₃)pyrrolo[2,1-f][1,2,4]triazin-2-amine [496];
1-((3R,4S)-4-((5-(1-(2,2-difluoroethyl)-2-methyl-1H-benzo[d]imidazol-6-yl)-6-fluoro-4-(methoxy-d₃)pyrrolo[2,1-f][1,2,4]triazin-2-yl)amino)-3-fluoropiperidin-1-yl)ethan-1-one [497];
1-((3R,4S)-4-((5-(1-(2,2-difluoroethyl)-2-methyl-1H-benzo[d]imidazol-6-yl)-6-fluoro-4-(methoxy-d₃)pyrrolo[2,1-f][1,2,4]triazin-2-yl)amino)-3-fluoropiperidin-1-yl)ethan-1-one-2,2,2-d₃ [498];
1-((3R,4S)-4-((5-(1-(2,2-difluoroethyl)-2-methyl-1H-benzo[d]imidazol-6-yl)-6-fluoro-4-(methoxy-d₃)pyrrolo[2,1-f][1,2,4]triazin-2-yl)amino)-3-fluoropiperidin-1-yl)-2-hydroxyethan-1-one [499]; and
5-(1-(2,2-difluoroethyl)-2-methyl-1H-benzo[d]imidazol-6-yl)-6-fluoro-N-((3S,4R)-3-fluoro-1-(oxetan-3-yl)piperidin-4-yl)-4-(methoxy-d₃)pyrrolo[2,1-f][1,2,4]triazin-2-amine [500]; or a pharmaceutically acceptable salt thereof.

22. The compound of claim 1, wherein the compound of Formula I is selected from the group consisting of
5-(1-(2,2-difluoroethyl)-2-methyl-1H-benzo[d]imidazol-6-yl)-6-fluoro-N-((3R,4R)-3-fluoro-1-(oxetan-3-yl)piperidin-4-yl)-4-(methoxy-d₃)pyrrolo[2,1-f][1,2,4]triazin-2-amine [501];
5-(1-(2,2-difluoroethyl)-2-methyl-1H-benzo[d]imidazol-6-yl)-6-fluoro-N-((3S,4S)-3-fluoro-1-(oxetan-3-yl)piperidin-4-yl)-4-(methoxy-d₃)pyrrolo[2,1-f][1,2,4]triazin-2-amine [502];
5-(1-(2,2-difluoroethyl)-2-methyl-1H-benzo[d]imidazol-6-yl)-6-fluoro-N-((3R,4S)-3-fluoro-1-(oxetan-3-yl)piperidin-4-yl)-4-(methoxy-d₃)pyrrolo[2,1-f][1,2,4]triazin-2-amine [503];
(R)—N-(3,3-difluoro-1-methylpiperidin-4-yl)-5-(1-(2,2-difluoroethyl)-2-methyl-1H-benzo[d]imidazol-6-yl)-6-fluoro-4-(methoxy-d₃)pyrrolo[2,1-f][1,2,4]triazin-2-amine [504];
(R)—N-(3,3-difluoro-1-(methyl-d₃)piperidin-4-yl)-5-(1-(2,2-difluoroethyl)-2-methyl-1H-benzo[d]imidazol-6-yl)-6-fluoro-4-(methoxy-d₃)pyrrolo[2,1-f][1,2,4]triazin-2-amine [505];
(R)-1-(4-((5-(1-(2,2-difluoroethyl)-2-methyl-1H-benzo[d]imidazol-6-yl)-6-fluoro-4-(methoxy-d₃)pyrrolo[2,1-f][1,2,4]triazin-2-yl)amino)-3,3-difluoropiperidin-1-yl)ethan-1-one [506];
(R)-1-(4-((5-(1-(2,2-difluoroethyl)-2-methyl-1H-benzo[d]imidazol-6-yl)-6-fluoro-4-(methoxy-d₃)pyrrolo[2,1-f][1,2,4]triazin-2-yl)amino)-3,3-difluoropiperidin-1-yl)ethan-1-one-2,2,2-d₃ [507];
(R)-1-(4-((5-(1-(2,2-difluoroethyl)-2-methyl-1H-benzo[d]imidazol-6-yl)-6-fluoro-4-(methoxy-d₃)pyrrolo[2,1-f][1,2,4]triazin-2-yl)amino)-3,3-difluoropiperidin-1-yl)-2-hydroxyethan-1-one [508];
(R)—N-(3,3-difluoro-1-(methylsulfonyl)piperidin-4-yl)-5-(1-(2,2-difluoroethyl)-2-methyl-1H-benzo[d]imidazol-6-yl)-6-fluoro-4-(methoxy-d₃)pyrrolo[2,1-f][1,2,4]triazin-2-amine [509];
(R)—N-(3,3-difluoro-1-(oxetan-3-yl)piperidin-4-yl)-5-(1-(2,2-difluoroethyl)-2-methyl-1H-benzo[d]imidazol-6-yl)-6-fluoro-4-(methoxy-d₃)pyrrolo[2,1-f][1,2,4]triazin-2-amine [510];
5-(1-(2,2-difluoroethyl)-2-methyl-1H-benzo[d]imidazol-6-yl)-6-fluoro-4-(methoxy-d₃)—N-(2-oxaspiro[3.5]nonan-7-yl)pyrrolo[2,1-f][1,2,4]triazin-2-amine [511];
5-(1-(2,2-difluoroethyl)-4-fluoro-2-methyl-1H-benzo[d]imidazol-6-yl)-N-((3S,4R)-3-fluoro-1-(2-methoxyethyl)piperidin-4-yl)-4-methoxypyrrolo[2,1-f][1,2,4]triazin-2-amine [512];
5-(1-(2,2-difluoroethyl)-4-fluoro-2-methyl-1H-benzo[d]imidazol-6-yl)-N-((3R,4R)-3-fluoro-1-(2-methoxyethyl)piperidin-4-yl)-4-methoxypyrrolo[2,1-f][1,2,4]triazin-2-amine [513];
5-(1-(2,2-difluoroethyl)-4-fluoro-2-methyl-1H-benzo[d]imidazol-6-yl)-N-((3S,4S)-3-fluoro-1-(2-methoxyethyl)piperidin-4-yl)-4-methoxypyrrolo[2,1-f][1,2,4]triazin-2-amine [514];
5-(1-(2,2-difluoroethyl)-4-fluoro-2-methyl-1H-benzo[d]imidazol-6-yl)-N-((3R,4S)-3-fluoro-1-(2-methoxyethyl)piperidin-4-yl)-4-methoxypyrrolo[2,1-f][1,2,4]triazin-2-amine [515];
5-(1-(2,2-difluoroethyl)-4-fluoro-2-methyl-1H-benzo[d]imidazol-6-yl)-N-((3S,4R)-3-fluoro-1-(oxetan-3-yl-3-d)piperidin-4-yl)-4-methoxypyrrolo[2,1-f][1,2,4]triazin-2-amine [516];
5-(1-(2,2-difluoroethyl)-4-fluoro-2-methyl-1H-benzo[d]imidazol-6-yl)-N-((3R,4R)-3-fluoro-1-(oxetan-3-yl-3-d)piperidin-4-yl)-4-methoxypyrrolo[2,1-f][1,2,4]triazin-2-amine [517];
5-(1-(2,2-difluoroethyl)-4-fluoro-2-methyl-1H-benzo[d]imidazol-6-yl)-N-((3S,4S)-3-fluoro-1-(oxetan-3-yl-3-d)piperidin-4-yl)-4-methoxypyrrolo[2,1-f][1,2,4]triazin-2-amine [518];
5-(1-(2,2-difluoroethyl)-4-fluoro-2-methyl-1H-benzo[d]imidazol-6-yl)-N-((3R,4S)-3-fluoro-1-(oxetan-3-yl-3-d)piperidin-4-yl)-4-methoxypyrrolo[2,1-f][1,2,4]triazin-2-amine [519];
(R)—N-(3,3-difluoro-1-(2-methoxyethyl)piperidin-4-yl)-5-(1-(2,2-difluoroethyl)-4-fluoro-2-methyl-1H-benzo[d]imidazol-6-yl)-4-methoxypyrrolo[2,1-f][1,2,4]triazin-2-amine [520];
(R)—N-(3,3-difluoro-1-(oxetan-3-yl-3-d)piperidin-4-yl)-5-(1-(2,2-difluoroethyl)-4-fluoro-2-methyl-1H-benzo[d]imidazol-6-yl)-4-methoxypyrrolo[2,1-f][1,2,4]triazin-2-amine [521];
3-((5-(1-(2,2-difluoroethyl)-4-fluoro-2-methyl-1H-benzo[d]imidazol-6-yl)-6-fluoro-4-methoxypyrrolo[2,1-f][1,2,4]triazin-2-yl)amino)-2,2-dimethylpropanenitrile [522];
(1S,4S)-4-((5-(1-(2,2-difluoroethyl)-4-fluoro-2-methyl-1H-benzo[d]imidazol-6-yl)-6-fluoro-4-methoxypyrrolo[2,1-f][1,2,4]triazin-2-yl)amino)-1-methylcyclohexan-1-ol [523];
(1R,4R)-4-((5-(1-(2,2-difluoroethyl)-4-fluoro-2-methyl-1H-benzo[d]imidazol-6-yl)-6-fluoro-4-methoxypyrrolo[2,1-f][1,2,4]triazin-2-yl)amino)-1-methylcyclohexan-1-ol [524];
(R)—N-(4,4-difluoro-1-methylpyrrolidin-3-yl)-5-(1-(2,2-difluoroethyl)-4-fluoro-2-methyl-1H-benzo[d]imidazol-6-yl)-6-fluoro-4-methoxypyrrolo[2,1-f][1,2,4]triazin-2-amine [525];
(R)-1-(4-((5-(1-(2,2-difluoroethyl)-4-fluoro-2-methyl-1H-benzo[d]imidazol-6-yl)-6-fluoro-4-methoxypyrrolo[2,1-f][1,2,4]triazin-2-yl)amino)-3,3-difluoropyrrolidin-1-yl)ethan-1-one [526];
(R)—N-(4,4-difluoro-1-(oxetan-3-yl)pyrrolidin-3-yl)-5-(1-(2,2-difluoroethyl)-4-fluoro-2-methyl-1H-benzo[d]imidazol-6-yl)-6-fluoro-4-methoxypyrrolo[2,1-f][1,2,4]triazin-2-amine [527];
5-(1-(2,2-difluoroethyl)-4-fluoro-2-methyl-1H-benzo[d]imidazol-6-yl)-6-fluoro-4-methoxy-N-(1-(oxetan-3-yl)piperidin-4-yl)pyrrolo[2,1-f][1,2,4]triazin-2-amine [528];
5-(1-(2,2-difluoroethyl)-4-fluoro-2-methyl-1H-benzo[d]imidazol-6-yl)-6-fluoro-N-((3S,4R)-3-fluoro-1-methylpiperidin-4-yl)-4-methoxypyrrolo[2,1-f][1,2,4]triazin-2-amine [529];
5-(1-(2,2-difluoroethyl)-4-fluoro-2-methyl-1H-benzo[d]imidazol-6-yl)-6-fluoro-N-((3R,4S)-3-fluoro-1-methylpiperidin-4-yl)-4-methoxypyrrolo[2,1-f][1,2,4]triazin-2-amine [530];
5-(1-(2,2-difluoroethyl)-4-fluoro-2-methyl-1H-benzo[d]imidazol-6-yl)-6-fluoro-N-((3S,4R)-3-fluoro-1-(2-methoxyethyl)piperidin-4-yl)-4-methoxypyrrolo[2,1-f][1,2,4]triazin-2-amine [531];
5-(1-(2,2-difluoroethyl)-4-fluoro-2-methyl-1H-benzo[d]imidazol-6-yl)-6-fluoro-N-((3R,4R)-3-fluoro-1-(2-methoxyethyl)piperidin-4-yl)-4-methoxypyrrolo[2,1-f][1,2,4]triazin-2-amine [532];
5-(1-(2,2-difluoroethyl)-4-fluoro-2-methyl-1H-benzo[d]imidazol-6-yl)-6-fluoro-N-((3S,4S)-3-fluoro-1-(2-methoxyethyl)piperidin-4-yl)-4-methoxypyrrolo[2,1-f][1,2,4]triazin-2-amine [533];
5-(1-(2,2-difluoroethyl)-4-fluoro-2-methyl-1H-benzo[d]imidazol-6-yl)-6-fluoro-N-((3R,4S)-3-fluoro-1-(2-methoxyethyl)piperidin-4-yl)-4-methoxypyrrolo[2,1-f][1,2,4]triazin-2-amine [534];
1-((3R,4S)-4-((5-(1-(2,2-difluoroethyl)-4-fluoro-2-methyl-1H-benzo[d]imidazol-6-yl)-6-fluoro-4-methoxypyrrolo[2,1-f][1,2,4]triazin-2-yl)amino)-3-fluoropiperidin-1-yl)ethan-1-one [535];
1-((3R,4S)-4-((5-(1-(2,2-difluoroethyl)-4-fluoro-2-methyl-1H-benzo[d]imidazol-6-yl)-6-fluoro-4-methoxypyrrolo[2,1-f][1,2,4]triazin-2-yl)amino)-3-fluoropiperidin-1-yl)ethan-1-one-2,2,2-d₃ [536];
1-((3R,4S)-4-((5-(1-(2,2-difluoroethyl)-4-fluoro-2-methyl-1H-benzo[d]imidazol-6-yl)-6-fluoro-4-methoxypyrrolo[2,1-f][1,2,4]triazin-2-yl)amino)-3-fluoropiperidin-1-yl)-2-hydroxyethan-1-one [537];
5-(1-(2,2-difluoroethyl)-4-fluoro-2-methyl-1H-benzo[d]imidazol-6-yl)-6-fluoro-N-((3S,4R)-3-fluoro-1-(oxetan-3-yl)piperidin-4-yl)-4-methoxypyrrolo[2,1-f][1,2,4]triazin-2-amine [538];
5-(1-(2,2-difluoroethyl)-4-fluoro-2-methyl-1H-benzo[d]imidazol-6-yl)-6-fluoro-N-((3R,4R)-3-fluoro-1-(oxetan-3-yl)piperidin-4-yl)-4-methoxypyrrolo[2,1-f][1,2,4]triazin-2-amine [539];
5-(1-(2,2-difluoroethyl)-4-fluoro-2-methyl-1H-benzo[d]imidazol-6-yl)-6-fluoro-N-((3S,4S)-3-fluoro-1-(oxetan-3-yl)piperidin-4-yl)-4-methoxypyrrolo[2,1-f][1,2,4]triazin-2-amine [540];
5-(1-(2,2-difluoroethyl)-4-fluoro-2-methyl-1H-benzo[d]imidazol-6-yl)-6-fluoro-N-((3R,4S)-3-fluoro-1-(oxetan-3-yl)piperidin-4-yl)-4-methoxypyrrolo[2,1-f][1,2,4]triazin-2-amine [541];
5-(1-(2,2-difluoroethyl)-4-fluoro-2-methyl-1H-benzo[d]imidazol-6-yl)-6-fluoro-N-((3S,4R)-3-fluoro-1-(oxetan-3-yl-3-d)piperidin-4-yl)-4-methoxypyrrolo[2,1-f][1,2,4]triazin-2-amine [542];
5-(1-(2,2-difluoroethyl)-4-fluoro-2-methyl-1H-benzo[d]imidazol-6-yl)-6-fluoro-N-((3R,4R)-3-fluoro-1-(oxetan-3-yl-3-d)piperidin-4-yl)-4-methoxypyrrolo[2,1-f][1,2,4]triazin-2-amine [543];
5-(1-(2,2-difluoroethyl)-4-fluoro-2-methyl-1H-benzo[d]imidazol-6-yl)-6-fluoro-N-((3S,4S)-3-fluoro-1-(oxetan-3-yl-3-d)piperidin-4-yl)-4-methoxypyrrolo[2,1-f][1,2,4]triazin-2-amine [544];
5-(1-(2,2-difluoroethyl)-4-fluoro-2-methyl-1H-benzo[d]imidazol-6-yl)-6-fluoro-N-((3R,4S)-3-fluoro-1-(oxetan-3-yl-3-d)piperidin-4-yl)-4-methoxypyrrolo[2,1-f][1,2,4]triazin-2-amine [545];
(R)—N-(3,3-difluoro-1-methylpiperidin-4-yl)-5-(1-(2,2-difluoroethyl)-4-fluoro-2-methyl-1H-benzo[d]imidazol-6-yl)-6-fluoro-4-methoxypyrrolo[2,1-f][1,2,4]triazin-2-amine [546];
(R)—N-(3,3-difluoro-1-(2-methoxyethyl)piperidin-4-yl)-5-(1-(2,2-difluoroethyl)-4-fluoro-2-methyl-1H-benzo[d]imidazol-6-yl)-6-fluoro-4-methoxypyrrolo[2,1-f][1,2,4]triazin-2-amine [547];
(R)—N-(3,3-difluoro-1-(methyl-d₃)piperidin-4-yl)-5-(1-(2,2-difluoroethyl)-4-fluoro-2-methyl-1H-benzo[d]imidazol-6-yl)-6-fluoro-4-methoxypyrrolo[2,1-f][1,2,4]triazin-2-amine [548];
(R)-1-(4-((5-(1-(2,2-difluoroethyl)-4-fluoro-2-methyl-1H-benzo[d]imidazol-6-yl)-6-fluoro-4-methoxypyrrolo[2,1-f][1,2,4]triazin-2-yl)amino)-3,3-difluoropiperidin-1-yl)ethan-1-one [549];
(R)-1-(4-((5-(1-(2,2-difluoroethyl)-4-fluoro-2-methyl-1H-benzo[d]imidazol-6-yl)-6-fluoro-4-methoxypyrrolo[2,1-f][1,2,4]triazin-2-yl)amino)-3,3-difluoropiperidin-1-yl)ethan-1-one-2,2,2-d₃ [550];
(R)-1-(4-((5-(1-(2,2-difluoroethyl)-4-fluoro-2-methyl-1H-benzo[d]imidazol-6-yl)-6-fluoro-4-methoxypyrrolo[2,1-f][1,2,4]triazin-2-yl)amino)-3,3-difluoropiperidin-1-yl)-2-hydroxyethan-1-one [551];
(R)—N-(3,3-difluoro-1-(methylsulfonyl)piperidin-4-yl)-5-(1-(2,2-difluoroethyl)-4-fluoro-2-methyl-1H-benzo[d]imidazol-6-yl)-6-fluoro-4-methoxypyrrolo[2,1-f][1,2,4]triazin-2-amine [552];
(R)—N-(3,3-difluoro-1-(oxetan-3-yl)piperidin-4-yl)-5-(1-(2,2-difluoroethyl)-4-fluoro-2-methyl-1H-benzo[d]imidazol-6-yl)-6-fluoro-4-methoxypyrrolo[2,1-f][1,2,4]triazin-2-amine [553];
(R)—N-(3,3-difluoro-1-(oxetan-3-yl-3-d)piperidin-4-yl)-5-(1-(2,2-difluoroethyl)-4-fluoro-2-methyl-1H-benzo[d]imidazol-6-yl)-6-fluoro-4-methoxypyrrolo[2,1-f][1,2,4]triazin-2-amine [554];
5-(1-(2,2-difluoroethyl)-4-fluoro-2-methyl-1H-benzo[d]imidazol-6-yl)-6-fluoro-4-methoxy-N-(2-oxaspiro[3.5]nonan-7-yl)pyrrolo[2,1-f][1,2,4]triazin-2-amine [555];
(1S,4S)-4-((5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-yl)amino)-1-methylcyclohexan-1-ol [556];
(1R,4R)-4-((5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-yl)amino)-1-methylcyclohexan-1-ol [557];
(R)—N-(4,4-difluoro-1-methylpyrrolidin-3-yl)-5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-amine [558];
(R)-1-(4-((5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-yl)amino)-3,3-difluoropyrrolidin-1-yl)ethan-1-one [559];
(R)—N-(4,4-difluoro-1-(oxetan-3-yl)pyrrolidin-3-yl)-5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-amine [560];
5-(1-(2,2-difluoroethyl)-4-fluoro-2-methyl-1H-benzo[d]imidazol-6-yl)-6-fluoro-4-(methoxy-d₃)—N-(1-(oxetan-3-yl)piperidin-4-yl)pyrrolo[2,1-f][1,2,4]triazin-2-amine [561];
5-(1-(2,2-difluoroethyl)-4-fluoro-2-methyl-1H-benzo[d]imidazol-6-yl)-6-fluoro-N-((3S,4R)-3-fluoro-1-methylpiperidin-4-yl)-4-(methoxy-d₃)pyrrolo[2,1-f][1,2,4]triazin-2-amine [562];
5-(1-(2,2-difluoroethyl)-4-fluoro-2-methyl-1H-benzo[d]imidazol-6-yl)-6-fluoro-N-((3R,4S)-3-fluoro-1-methylpiperidin-4-yl)-4-(methoxy-d₃)pyrrolo[2,1-f][1,2,4]triazin-2-amine [563];
1-((3R,4S)-4-((5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-yl)amino)-3-fluoropiperidin-1-yl)ethan-1-one [564];
1-((3R,4S)-4-((5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-yl)amino)-3-fluoropiperidin-1-yl)ethan-1-one-2,2,2-d₃ [565];
1-((3R,4S)-4-((5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-yl)amino)-3-fluoropiperidin-1-yl)-2-hydroxyethan-1-one [566];
5-(1-(2,2-difluoroethyl)-4-fluoro-2-methyl-1H-benzo[d]imidazol-6-yl)-6-fluoro-N-((3S,4R)-3-fluoro-1-(oxetan-3-yl)piperidin-4-yl)-4-(methoxy-d₃)pyrrolo[2,1-f][1,2,4]triazin-2-amine [567];
5-(1-(2,2-difluoroethyl)-4-fluoro-2-methyl-1H-benzo[d]imidazol-6-yl)-6-fluoro-N-((3R,4R)-3-fluoro-1-(oxetan-3-yl)piperidin-4-yl)-4-(methoxy-d₃)pyrrolo[2,1-f][1,2,4]triazin-2-amine [568];
5-(1-(2,2-difluoroethyl)-4-fluoro-2-methyl-1H-benzo[d]imidazol-6-yl)-6-fluoro-N-((3S,4S)-3-fluoro-1-(oxetan-3-yl)piperidin-4-yl)-4-(methoxy-d₃)pyrrolo[2,1-f][1,2,4]triazin-2-amine [569];
5-(1-(2,2-difluoroethyl)-4-fluoro-2-methyl-1H-benzo[d]imidazol-6-yl)-6-fluoro-N-((3R,4S)-3-fluoro-1-(oxetan-3-yl)piperidin-4-yl)-4-(methoxy-d₃)pyrrolo[2,1-f][1,2,4]triazin-2-amine [570];
(R)—N-(3,3-difluoro-1-methylpiperidin-4-yl)-5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-amine [571];
(R)—N-(3,3-difluoro-1-(methyl-d₃)piperidin-4-yl)-5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-amine [572];
(R)-1-(4-((5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-yl)amino)-3,3-difluoropiperidin-1-yl)ethan-1-one [573];
(R)-1-(4-((5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-yl)amino)-3,3-difluoropiperidin-1-yl)ethan-1-one-2,2,2-d₃ [574];
(R)-1-(4-((5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-yl)amino)-3,3-difluoropiperidin-1-yl)-2-hydroxyethan-1-one [575];
(R)—N-(3,3-difluoro-1-(methylsulfonyl)piperidin-4-yl)-5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-amine [576];
(R)—N-(3,3-difluoro-1-(oxetan-3-yl)piperidin-4-yl)-5-(1-(2,2-difluoroethyl)-4-fluoro-2-methyl-1H-benzo[d]imidazol-6-yl)-6-fluoro-4-(methoxy-d₃)pyrrolo[2,1-f][1,2,4]triazin-2-amine [577];
5-(1-(2,2-difluoroethyl)-4-fluoro-2-methyl-1H-benzo[d]imidazol-6-yl)-6-fluoro-4-(methoxy-d₃)—N-(2-oxaspiro[3.5]nonan-7-yl)pyrrolo[2,1-f][1,2,4]triazin-2-amine [578];
3-((4-methoxy-5-(2-methyl-1-(2,2,2-trifluoroethyl)-1H-benzo[d]imidazol-6-yl)pyrrolo[2,1-f][1,2,4]triazin-2-yl)amino)-2,2-dimethylpropanenitrile [579];
(1S,4S)-4-((4-methoxy-5-(2-methyl-1-(2,2,2-trifluoroethyl)-1H-benzo[d]imidazol-6-yl)pyrrolo[2,1-f][1,2,4]triazin-2-yl)amino)-1-methylcyclohexan-1-ol [580];
(1R,4R)-4-((4-methoxy-5-(2-methyl-1-(2,2,2-trifluoroethyl)-1H-benzo[d]imidazol-6-yl)pyrrolo[2,1-f][1,2,4]triazin-2-yl)amino)-1-methylcyclohexan-1-ol [581];
(R)—N-(4,4-difluoro-1-methylpyrrolidin-3-yl)-4-methoxy-5-(2-methyl-1-(2,2,2-trifluoroethyl)-1H-benzo[d]imidazol-6-yl)pyrrolo[2,1-f][1,2,4]triazin-2-amine [582];
(R)-1-(3,3-difluoro-4-((4-methoxy-5-(2-methyl-1-(2,2,2-trifluoroethyl)-1H-benzo[d]imidazol-6-yl)pyrrolo[2,1-f][1,2,4]triazin-2-yl)amino)pyrrolidin-1-yl)ethan-1-one [583];
(R)—N-(4,4-difluoro-1-(oxetan-3-yl)pyrrolidin-3-yl)-4-methoxy-5-(2-methyl-1-(2,2,2-trifluoroethyl)-1H-benzo[d]imidazol-6-yl)pyrrolo[2,1-f][1,2,4]triazin-2-amine [584];
4-methoxy-5-(2-methyl-1-(2,2,2-trifluoroethyl)-1H-benzo[d]imidazol-6-yl)-N-(1-(oxetan-3-yl)piperidin-4-yl)pyrrolo[2,1-f][1,2,4]triazin-2-amine [585];
N-((3S,4R)-3-fluoro-1-methylpiperidin-4-yl)-4-methoxy-5-(2-methyl-1-(2,2,2-trifluoroethyl)-1H-benzo[d]imidazol-6-yl)pyrrolo[2,1-f][1,2,4]triazin-2-amine [586];
N-((3R,4S)-3-fluoro-1-methylpiperidin-4-yl)-4-methoxy-5-(2-methyl-1-(2,2,2-trifluoroethyl)-1H-benzo[d]imidazol-6-yl)pyrrolo[2,1-f][1,2,4]triazin-2-amine [587];
1-((3R,4S)-3-fluoro-4-((4-methoxy-5-(2-methyl-1-(2,2,2-trifluoroethyl)-1H-benzo[d]imidazol-6-yl)pyrrolo[2,1-f][1,2,4]triazin-2-yl)amino)piperidin-1-yl)ethan-1-one [588];
1-((3R,4S)-3-fluoro-4-((4-methoxy-5-(2-methyl-1-(2,2,2-trifluoroethyl)-1H-benzo[d]imidazol-6-yl)pyrrolo[2,1-f][1,2,4]triazin-2-yl)amino)piperidin-1-yl)ethan-1-one-2,2,2-d₃ [589];
1-((3R,4S)-3-fluoro-4-((4-methoxy-5-(2-methyl-1-(2,2,2-trifluoroethyl)-1H-benzo[d]imidazol-6-yl)pyrrolo[2,1-f][1,2,4]triazin-2-yl)amino)piperidin-1-yl)-2-hydroxyethan-1-one [590];
N-((3S,4R)-3-fluoro-1-(oxetan-3-yl)piperidin-4-yl)-4-methoxy-5-(2-methyl-1-(2,2,2-trifluoroethyl)-1H-benzo[d]imidazol-6-yl)pyrrolo[2,1-f][1,2,4]triazin-2-amine [591];
N-((3R,4R)-3-fluoro-1-(oxetan-3-yl)piperidin-4-yl)-4-methoxy-5-(2-methyl-1-(2,2,2-trifluoroethyl)-1H-benzo[d]imidazol-6-yl)pyrrolo[2,1-f][1,2,4]triazin-2-amine [592];
N-((3S,4S)-3-fluoro-1-(oxetan-3-yl)piperidin-4-yl)-4-methoxy-5-(2-methyl-1-(2,2,2-trifluoroethyl)-1H-benzo[d]imidazol-6-yl)pyrrolo[2,1-f][1,2,4]triazin-2-amine [593];
N-((3R,4S)-3-fluoro-1-(oxetan-3-yl)piperidin-4-yl)-4-methoxy-5-(2-methyl-1-(2,2,2-trifluoroethyl)-1H-benzo[d]imidazol-6-yl)pyrrolo[2,1-f][1,2,4]triazin-2-amine [594];
(R)—N-(3,3-difluoro-1-methylpiperidin-4-yl)-4-methoxy-5-(2-methyl-1-(2,2,2-trifluoroethyl)-1H-benzo[d]imidazol-6-yl)pyrrolo[2,1-f][1,2,4]triazin-2-amine [595];
(R)—N-(3,3-difluoro-1-(methyl-d₃)piperidin-4-yl)-4-methoxy-5-(2-methyl-1-(2,2,2-trifluoroethyl)-1H-benzo[d]imidazol-6-yl)pyrrolo[2,1-f][1,2,4]triazin-2-amine [596];
(R)-1-(3,3-difluoro-4-((4-methoxy-5-(2-methyl-1-(2,2,2-trifluoroethyl)-1H-benzo[d]imidazol-6-yl)pyrrolo[2,1-f][1,2,4]triazin-2-yl)amino)piperidin-1-yl)ethan-1-one [597];
(R)-1-(3,3-difluoro-4-((4-methoxy-5-(2-methyl-1-(2,2,2-trifluoroethyl)-1H-benzo[d]imidazol-6-yl)pyrrolo[2,1-f][1,2,4]triazin-2-yl)amino)piperidin-1-yl)ethan-1-one-2,2,2-d₃ [598];
(R)-1-(3,3-difluoro-4-((4-methoxy-5-(2-methyl-1-(2,2,2-trifluoroethyl)-1H-benzo[d]imidazol-6-yl)pyrrolo[2,1-f][1,2,4]triazin-2-yl)amino)piperidin-1-yl)-2-hydroxyethan-1-one [599]; and
(R)—N-(3,3-difluoro-1-(methylsulfonyl)piperidin-4-yl)-4-methoxy-5-(2-methyl-1-(2,2,2-trifluoroethyl)-1H-benzo[d]imidazol-6-yl)pyrrolo[2,1-f][1,2,4]triazin-2-amine [600]; or a pharmaceutically acceptable salt thereof.

23. The compound of claim 1, wherein the compound of Formula I is selected from the group consisting of:
(R)—N-(3,3-difluoro-1-(oxetan-3-yl)piperidin-4-yl)-4-methoxy-5-(2-methyl-1-(2,2,2-trifluoroethyl)-1H-benzo[d]imidazol-6-yl)pyrrolo[2,1-f][1,2,4]triazin-2-amine [601];
4-methoxy-5-(2-methyl-1-(2,2,2-trifluoroethyl)-1H-benzo[d]imidazol-6-yl)-N-(2-oxaspiro[3.5]nonan-7-yl)pyrrolo[2,1-f][1,2,4]triazin-2-amine [602];
(R)-1-(3,3-difluoro-4-((4-methoxy-5-(2-methylbenzo[d]oxazol-6-yl)pyrrolo[2,1-f][1,2,4]triazin-2-yl)amino)piperidin-1-yl)ethan-1-one [603];
(R)-1-(3,3-difluoro-4-((4-methoxy-5-(2-methylbenzo[d]thiazol-6-yl)pyrrolo[2,1-f][1,2,4]triazin-2-yl)amino)piperidin-1-yl)ethan-1-one [604];
(1S,3S)-3-((5-([1,2,4]triazolo[1,5-a]pyridin-6-yl)-4-methoxypyrrolo[2,1-f][1,2,4]triazin-2-yl)amino)-1-methylcyclobutan-1-ol [605];
(1R,3R)-3-((5-([1,2,4]triazolo[1,5-a]pyridin-6-yl)-4-methoxypyrrolo[2,1-f][1,2,4]triazin-2-yl)amino)-1-methylcyclobutan-1-ol [606];
N-((1R,3R)-3-((5-([1,2,4]triazolo[1,5-a]pyridin-6-yl)-4-methoxypyrrolo[2,1-f][1,2,4]triazin-2-yl)amino)-1-methylcyclobutyl)-2-hydroxyacetamide [607];
(1R,3S)-1-ethyl-3-((4-methoxy-5-(1-methyl-1H-benzo[d][1,2,3]triazol-6-yl)pyrrolo[2,1-f][1,2,4]triazin-2-yl)amino)cyclobutan-1-ol [608];
(1R,3S)-1-ethyl-3-((5-(1-ethyl-1H-benzo[d][1,2,3]triazol-6-yl)-4-methoxypyrrolo[2,1-f][1,2,4]triazin-2-yl)amino)cyclobutan-1-ol [609];
3-((5-(1-(2-fluoroethyl)-1H-benzo[d][1,2,3]triazol-6-yl)-4-methoxypyrrolo[2,1-f][1,2,4]triazin-2-yl)amino)-2,2-dimethylpropanenitrile [610];
(1S,3R)-1-ethyl-3-((5-(1-(2-fluoroethyl)-1H-benzo[d][1,2,3]triazol-6-yl)-4-methoxypyrrolo[2,1-f][1,2,4]triazin-2-yl)amino)cyclobutan-1-ol [611];
N-((1S,4S)-4-((5-(1-(2-fluoroethyl)-1H-benzo[d][1,2,3]triazol-6-yl)-4-methoxypyrrolo[2,1-f][1,2,4]triazin-2-yl)amino)cyclohexyl)acetamide [612];
1-((3S,4R)-3-fluoro-4-((5-(1-(2-fluoroethyl)-1H-benzo[d][1,2,3]triazol-6-yl)-4-methoxypyrrolo[2,1-f][1,2,4]triazin-2-yl)amino)pyrrolidin-1-yl)propan-1-one [613];
(R)—N-(4,4-difluoro-1-methylpyrrolidin-3-yl)-5-(1-(2-fluoroethyl)-1H-benzo[d][1,2,3]triazol-6-yl)-4-methoxypyrrolo[2,1-f][1,2,4]triazin-2-amine [614];
(R)—N-(4,4-difluoro-1-(oxetan-3-yl)pyrrolidin-3-yl)-5-(1-(2-fluoroethyl)-1H-benzo[d][1,2,3]triazol-6-yl)-4-methoxypyrrolo[2,1-f][1,2,4]triazin-2-amine [615];
2-((3R,4S)-3-fluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-ol [616];
N-((3S,4R)-3-fluoro-1-(2-methoxyethyl)piperidin-4-yl)-5-(1-(2-fluoroethyl)-1H-benzo[d][1,2,3]triazol-6-yl)-4-methoxypyrrolo[2,1-f][1,2,4]triazin-2-amine [617];
N-((3R,4R)-3-fluoro-1-(2-methoxyethyl)piperidin-4-yl)-5-(1-(2-fluoroethyl)-1H-benzo[d][1,2,3]triazol-6-yl)-4-methoxypyrrolo[2,1-f][1,2,4]triazin-2-amine [618];
N-((3S,4S)-3-fluoro-1-(2-methoxyethyl)piperidin-4-yl)-5-(1-(2-fluoroethyl)-1H-benzo[d][1,2,3]triazol-6-yl)-4-methoxypyrrolo[2,1-f][1,2,4]triazin-2-amine [619];
N-((3R,4S)-3-fluoro-1-(2-methoxyethyl)piperidin-4-yl)-5-(1-(2-fluoroethyl)-1H-benzo[d][1,2,3]triazol-6-yl)-4-methoxypyrrolo[2,1-f][1,2,4]triazin-2-amine [620];
2-((3R,4S)-3-fluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)-N,N-dimethylacetamide [621];
1-(azetidin-1-yl)-2-((3R,4S)-3-fluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-one [622];
1-((3R,4S)-3-fluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [623];
N-((3S,4R)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-2-amine [624];
N-((3R,4R)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-2-amine [625];
N-((3S,4S)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-2-amine [626];
N-((3R,4S)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-2-amine [627];
N-((3S,4R)-3-fluoro-1-(oxetan-3-yl-3-d)piperidin-4-yl)-5-(1-(2-fluoroethyl)-1H-benzo[d][1,2,3]triazol-6-yl)-4-methoxypyrrolo[2,1-f][1,2,4]triazin-2-amine [628];
N-((3R,4R)-3-fluoro-1-(oxetan-3-yl-3-d)piperidin-4-yl)-5-(1-(2-fluoroethyl)-1H-benzo[d][1,2,3]triazol-6-yl)-4-methoxypyrrolo[2,1-f][1,2,4]triazin-2-amine [629];
N-((3S,4S)-3-fluoro-1-(oxetan-3-yl-3-d)piperidin-4-yl)-5-(1-(2-fluoroethyl)-1H-benzo[d][1,2,3]triazol-6-yl)-4-methoxypyrrolo[2,1-f][1,2,4]triazin-2-amine [630];
N-((3R,4S)-3-fluoro-1-(oxetan-3-yl-3-d)piperidin-4-yl)-5-(1-(2-fluoroethyl)-1H-benzo[d][1,2,3]triazol-6-yl)-4-methoxypyrrolo[2,1-f][1,2,4]triazin-2-amine [631];
(R)—N-(3,3-difluoro-1-methylpiperidin-4-yl)-5-(1-(2-fluoroethyl)-1H-benzo[d][1,2,3]triazol-6-yl)-4-methoxypyrrolo[2,1-f][1,2,4]triazin-2-amine [632];
(R)-2-(3,3-difluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-ol [633];
(R)—N-(3,3-difluoro-1-(2-methoxyethyl)piperidin-4-yl)-5-(1-(2-fluoroethyl)-1H-benzo[d][1,2,3]triazol-6-yl)-4-methoxypyrrolo[2,1-f][1,2,4]triazin-2-amine [634];
(R)-1-(azetidin-1-yl)-2-(3,3-difluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-one [635];
(R)-1-(3,3-difluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)-2-(dimethylamino)ethan-1-one [636];
(R)-1-(3,3-difluoro-4-((5-(1-(2-fluoroethyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [637];
(R)—N-(3,3-difluoro-1-(oxetan-3-yl-3-d)piperidin-4-yl)-5-(1-(2-fluoroethyl)-1H-benzo[d][1,2,3]triazol-6-yl)-4-methoxypyrrolo[2,1-f][1,2,4]triazin-2-amine [638];
(1S,4S)-4-((5-(1-(2-fluoroethyl)-1H-benzo[d][1,2,3]triazol-6-yl)-4-methoxypyrrolo[2,1-f][1,2,4]triazin-2-yl-7-d)amino)-1-methylcyclohexan-1-ol [639];
(1R,4R)-4-((5-(1-(2-fluoroethyl)-1H-benzo[d][1,2,3]triazol-6-yl)-4-methoxypyrrolo[2,1-f][1,2,4]triazin-2-yl-7-d)amino)-1-methylcyclohexan-1-ol [640];
(R)—N-(4,4-difluoro-1-methylpyrrolidin-3-yl)-5-(1-(2-fluoroethyl)-1H-benzo[d][1,2,3]triazol-6-yl)-4-methoxypyrrolo[2,1-f][1,2,4]triazin-7-d-2-amine [641];
(R)-1-(3,3-difluoro-4-((5-(1-(2-fluoroethyl)-1H-benzo[d][1,2,3]triazol-6-yl)-4-methoxypyrrolo[2,1-f][1,2,4]triazin-2-yl-7-d)amino)pyrrolidin-1-yl)ethan-1-one [642];
(R)—N-(4,4-difluoro-1-(oxetan-3-yl)pyrrolidin-3-yl)-5-(1-(2-fluoroethyl)-1H-benzo[d][1,2,3]triazol-6-yl)-4-methoxypyrrolo[2,1-f][1,2,4]triazin-7-d-2-amine [643];
5-(1-(2-fluoroethyl)-1H-benzo[d][1,2,3]triazol-6-yl)-4-methoxy-N-(1-(oxetan-3-yl)piperidin-4-yl)pyrrolo[2,1-f][1,2,4]triazin-7-d-2-amine [644];
N-((3S,4R)-3-fluoro-1-methylpiperidin-4-yl)-5-(1-(2-fluoroethyl)-1H-benzo[d][1,2,3]triazol-6-yl)-4-methoxypyrrolo[2,1-f][1,2,4]triazin-7-d-2-amine [645];
N-((3R,4S)-3-fluoro-1-methylpiperidin-4-yl)-5-(1-(2-fluoroethyl)-1H-benzo[d][1,2,3]triazol-6-yl)-4-methoxypyrrolo[2,1-f][1,2,4]triazin-7-d-2-amine [646];
1-((3R,4S)-3-fluoro-4-((5-(1-(2-fluoroethyl)-1H-benzo[d][1,2,3]triazol-6-yl)-4-methoxypyrrolo[2,1-f][1,2,4]triazin-2-yl-7-d)amino)piperidin-1-yl)ethan-1-one [647];
1-((3R,4S)-3-fluoro-4-((5-(1-(2-fluoroethyl)-1H-benzo[d][1,2,3]triazol-6-yl)-4-methoxypyrrolo[2,1-f][1,2,4]triazin-2-yl-7-d)amino)piperidin-1-yl)ethan-1-one-2,2,2-d₃ [648];
1-((3R,4S)-3-fluoro-4-((5-(1-(2-fluoroethyl)-1H-benzo[d][1,2,3]triazol-6-yl)-4-methoxypyrrolo[2,1-f][1,2,4]triazin-2-yl-7-d)amino)piperidin-1-yl)-2-hydroxyethan-1-one [649];
N-((3S,4R)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-7-d-2-amine [650];
N-((3R,4R)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-7-d-2-amine [651];
N-((3S,4S)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-7-d-2-amine [652];
N-((3R,4S)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-7-d-2-amine [653];
(R)—N-(3,3-difluoro-1-methylpiperidin-4-yl)-5-(1-(2-fluoroethyl)-1H-benzo[d][1,2,3]triazol-6-yl)-4-methoxypyrrolo[2,1-f][1,2,4]triazin-7-d-2-amine [654];
(R)—N-(3,3-difluoro-1-(methyl-d₃)piperidin-4-yl)-5-(1-(2-fluoroethyl)-1H-benzo[d][1,2,3]triazol-6-yl)-4-methoxypyrrolo[2,1-f][1,2,4]triazin-7-d-2-amine [655];
(R)-1-(3,3-difluoro-4-((5-(1-(2-fluoroethyl)-1H-benzo[d][1,2,3]triazol-6-yl)-4-methoxypyrrolo[2,1-f][1,2,4]triazin-2-yl-7-d)amino)piperidin-1-yl)ethan-1-one [656];
(R)-1-(3,3-difluoro-4-((5-(1-(2-fluoroethyl)-1H-benzo[d][1,2,3]triazol-6-yl)-4-methoxypyrrolo[2,1-f][1,2,4]triazin-2-yl-7-d)amino)piperidin-1-yl)ethan-1-one-2,2,2-d₃ [657];
(R)-1-(3,3-difluoro-4-((5-(1-(2-fluoroethyl)-1H-benzo[d][1,2,3]triazol-6-yl)-4-methoxypyrrolo[2,1-f][1,2,4]triazin-2-yl-7-d)amino)piperidin-1-yl)-2-hydroxyethan-1-one [658];
(R)—N-(3,3-difluoro-1-(methylsulfonyl)piperidin-4-yl)-5-(1-(2-fluoroethyl)-1H-benzo[d][1,2,3]triazol-6-yl)-4-methoxypyrrolo[2,1-f][1,2,4]triazin-7-d-2-amine [659];
(R)—N-(3,3-difluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-7-d-2-amine [660];
5-(1-(2-fluoroethyl)-1H-benzo[d][1,2,3]triazol-6-yl)-4-methoxy-N-(2-oxaspiro[3.5]nonan-7-yl)pyrrolo[2,1-f][1,2,4]triazin-7-d-2-amine [661];
(1S,4S)-4-((5-(1-(2-fluoroethyl)-1H-benzo[d][1,2,3]triazol-6-yl)-4-(methoxy-d₃)pyrrolo[2,1-f][1,2,4]triazin-2-yl)amino)-1-methylcyclohexan-1-ol [662];
(1R,4R)-4-((5-(1-(2-fluoroethyl)-1H-benzo[d][1,2,3]triazol-6-yl)-4-(methoxy-d₃)pyrrolo[2,1-f][1,2,4]triazin-2-yl)amino)-1-methylcyclohexan-1-ol [663];
(R)—N-(4,4-difluoro-1-methylpyrrolidin-3-yl)-5-(1-(2-fluoroethyl)-1H-benzo[d][1,2,3]triazol-6-yl)-4-(methoxy-d₃)pyrrolo[2,1-f][1,2,4]triazin-2-amine [664];
(R)-1-(3,3-difluoro-4-((5-(1-(2-fluoroethyl)-1H-benzo[d][1,2,3]triazol-6-yl)-4-(methoxy-d₃)pyrrolo[2,1-f][1,2,4]triazin-2-yl)amino)pyrrolidin-1-yl)ethan-1-one [665];
(R)—N-(4,4-difluoro-1-(oxetan-3-yl)pyrrolidin-3-yl)-5-(1-(2-fluoroethyl)-1H-benzo[d][1,2,3]triazol-6-yl)-4-(methoxy-d₃)pyrrolo[2,1-f][1,2,4]triazin-2-amine [666];
5-(1-(2-fluoroethyl)-1H-benzo[d][1,2,3]triazol-6-yl)-4-(methoxy-d₃)—N-(1-(oxetan-3-yl)piperidin-4-yl)pyrrolo[2,1-f][1,2,4]triazin-2-amine [667];
N-((3S,4R)-3-fluoro-1-methylpiperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [668];
N-((3R,4S)-3-fluoro-1-methylpiperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [669];
1-((3R,4S)-3-fluoro-4-((5-(1-(2-fluoroethyl)-1H-benzo[d][1,2,3]triazol-6-yl)-4-(methoxy-d₃)pyrrolo[2,1-f][1,2,4]triazin-2-yl)amino)piperidin-1-yl)ethan-1-one [670];
1-((3R,4S)-3-fluoro-4-((5-(1-(2-fluoroethyl)-1H-benzo[d][1,2,3]triazol-6-yl)-4-(methoxy-d₃)pyrrolo[2,1-f][1,2,4]triazin-2-yl)amino)piperidin-1-yl)ethan-1-one-2,2,2-d₃ [671];
1-((3R,4S)-3-fluoro-4-((5-(1-(2-fluoroethyl)-1H-benzo[d][1,2,3]triazol-6-yl)-4-(methoxy-d₃)pyrrolo[2,1-f][1,2,4]triazin-2-yl)amino)piperidin-1-yl)-2-hydroxyethan-1-one [672];
N-((3S,4R)-3-fluoro-1-(oxetan-3-yl)piperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [673];
N-((3R,4R)-3-fluoro-1-(oxetan-3-yl)piperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [674];
N-((3S,4S)-3-fluoro-1-(oxetan-3-yl)piperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [675];
N-((3R,4S)-3-fluoro-1-(oxetan-3-yl)piperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [676];
(R)—N-(3,3-difluoro-1-methylpiperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [677];
(R)—N-(3,3-difluoro-1-(methyl-d₃)piperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [678];
(R)-1-(3,3-difluoro-4-((5-(1-(2-fluoroethyl)-1H-benzo[d][1,2,3]triazol-6-yl)-4-(methoxy-d₃)pyrrolo[2,1-f][1,2,4]triazin-2-yl)amino)piperidin-1-yl)ethan-1-one [679];
(R)-1-(3,3-difluoro-4-((5-(1-(2-fluoroethyl)-1H-benzo[d][1,2,3]triazol-6-yl)-4-(methoxy-d₃)pyrrolo[2,1-f][1,2,4]triazin-2-yl)amino)piperidin-1-yl)ethan-1-one-2,2,2-d₃ [680];
(R)-1-(3,3-difluoro-4-((5-(1-(2-fluoroethyl)-1H-benzo[d][1,2,3]triazol-6-yl)-4-(methoxy-d₃)pyrrolo[2,1-f][1,2,4]triazin-2-yl)amino)piperidin-1-yl)-2-hydroxyethan-1-one [681];
(R)—N-(3,3-difluoro-1-(methylsulfonyl)piperidin-4-yl)-5-(1-(2-fluoroethyl)-1H-benzo[d][1,2,3]triazol-6-yl)-4-(methoxy-d)pyrrolo[2,1-f][1,2,4]triazin-2-amine [682];
(R)—N-(3,3-difluoro-1-(oxetan-3-yl)piperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [683];
5-(1-(2-fluoroethyl)-1H-benzo[d][1,2,3]triazol-6-yl)-4-(methoxy-d₃)—N-(2-oxaspiro[3.5]nonan-7-yl)pyrrolo[2,1-f][1,2,4]triazin-2-amine [684];
3-((6-fluoro-5-(1-(2-fluoroethyl)-1H-benzo[d][1,2,3]triazol-6-yl)-4-methoxypyrrolo[2,1-f][1,2,4]triazin-2-yl)amino)-2,2-dimethylpropanenitrile [685];
(1r,4r)-4-((6-fluoro-5-(1-(2-fluoroethyl)-1H-benzo[d][1,2,3]triazol-6-yl)-4-methoxypyrrolo[2,1-f][1,2,4]triazin-2-yl)amino)-1-methylcyclohexan-1-ol [686];
1-((3S,4R)-3-fluoro-4-((6-fluoro-5-(1-(2-fluoroethyl)-1H-benzo[d][1,2,3]triazol-6-yl)-4-methoxypyrrolo[2,1-f][1,2,4]triazin-2-yl)amino)pyrrolidin-1-yl)ethan-1-one [687];
6-fluoro-N-((3R,4S)-4-fluoro-1-(oxetan-3-yl)pyrrolidin-3-yl)-5-(1-(2-fluoroethyl)-1H-benzo[d][1,2,3]triazol-6-yl)-4-methoxypyrrolo[2,1-f][1,2,4]triazin-2-amine [688];
(R)—N-(4,4-difluoro-1-methylpyrrolidin-3-yl)-6-fluoro-5-(1-(2-fluoroethyl)-1H-benzo[d][1,2,3]triazol-6-yl)-4-methoxypyrrolo[2,1-f][1,2,4]triazin-2-amine [689];
(R)-1-(3,3-difluoro-4-((6-fluoro-5-(1-(2-fluoroethyl)-1H-benzo[d][1,2,3]triazol-6-yl)-4-methoxypyrrolo[2,1-f][1,2,4]triazin-2-yl)amino)pyrrolidin-1-yl)ethan-1-one [690];
(R)—N-(4,4-difluoro-1-(oxetan-3-yl)pyrrolidin-3-yl)-6-fluoro-5-(1-(2-fluoroethyl)-1H-benzo[d][1,2,3]triazol-6-yl)-4-methoxypyrrolo[2,1-f][1,2,4]triazin-2-amine [691];
6-fluoro-5-(1-(2-fluoroethyl)-1H-benzo[d][1,2,3]triazol-6-yl)-4-methoxy-N-(1-(oxetan-3-yl)piperidin-4-yl)pyrrolo[2,1-f][1,2,4]triazin-2-amine [692];
6-fluoro-N-((3S,4R)-3-fluoro-1-methylpiperidin-4-yl)-5-(1-(2-fluoroethyl)-1H-benzo[d][1,2,3]triazol-6-yl)-4-methoxypyrrolo[2,1-f][1,2,4]triazin-2-amine [693];
6-fluoro-N-((3R,4S)-3-fluoro-1-methylpiperidin-4-yl)-5-(1-(2-fluoroethyl)-1H-benzo[d][1,2,3]triazol-6-yl)-4-methoxypyrrolo[2,1-f][1,2,4]triazin-2-amine [694];
6-fluoro-N-((3S,4R)-3-fluoro-1-(2-methoxyethyl)piperidin-4-yl)-5-(1-(2-fluoroethyl)-1H-benzo[d][1,2,3]triazol-6-yl)-4-methoxypyrrolo[2,1-f][1,2,4]triazin-2-amine [695];
6-fluoro-N-((3R,4R)-3-fluoro-1-(2-methoxyethyl)piperidin-4-yl)-5-(1-(2-fluoroethyl)-1H-benzo[d][1,2,3]triazol-6-yl)-4-methoxypyrrolo[2,1-f][1,2,4]triazin-2-amine [696];
6-fluoro-N-((3S,4S)-3-fluoro-1-(2-methoxyethyl)piperidin-4-yl)-5-(1-(2-fluoroethyl)-1H-benzo[d][1,2,3]triazol-6-yl)-4-methoxypyrrolo[2,1-f][1,2,4]triazin-2-amine [697];
6-fluoro-N-((3R,4S)-3-fluoro-1-(2-methoxyethyl)piperidin-4-yl)-5-(1-(2-fluoroethyl)-1H-benzo[d][1,2,3]triazol-6-yl)-4-methoxypyrrolo[2,1-f][1,2,4]triazin-2-amine [698];
1-((3R,4S)-3-fluoro-4-((6-fluoro-5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-one [699]; and
1-((3R,4S)-3-fluoro-4-((6-fluoro-5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [700]; or
a pharmaceutically acceptable salt thereof.

24. The compound of claim 1, wherein the compound of Formula I is selected from the group consisting of:
1-((3R,4S)-3-fluoro-4-((6-fluoro-5-(1-(2-fluoroethyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [701];
6-fluoro-N-((3S,4R)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-2-amine [702];
6-fluoro-N-((3R,4R)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-2-amine [703];
6-fluoro-N-((3S,4S)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-2-amine [704];
6-fluoro-N-((3R,4S)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-2-amine [705];
6-fluoro-N-((3S,4R)-3-fluoro-1-(oxetan-3-yl-3-d)piperidin-4-yl)-5-(1-(2-fluoroethyl)-1H-benzo[d][1,2,3]triazol-6-yl)-4-methoxypyrrolo[2,1-f][1,2,4]triazin-2-amine [706];
6-fluoro-N-((3R,4R)-3-fluoro-1-(oxetan-3-yl-3-d)piperidin-4-yl)-5-(1-(2-fluoroethyl)-1H-benzo[d][1,2,3]triazol-6-yl)-4-methoxypyrrolo[2,1-f][1,2,4]triazin-2-amine [707];
6-fluoro-N-((3S,4S)-3-fluoro-1-(oxetan-3-yl-3-d)piperidin-4-yl)-5-(1-(2-fluoroethyl)-1H-benzo[d][1,2,3]triazol-6-yl)-4-methoxypyrrolo[2,1-f][1,2,4]triazin-2-amine [708];
6-fluoro-N-((3R,4S)-3-fluoro-1-(oxetan-3-yl-3-d)piperidin-4-yl)-5-(1-(2-fluoroethyl)-1H-benzo[d][1,2,3]triazol-6-yl)-4-methoxypyrrolo[2,1-f][1,2,4]triazin-2-amine [709];
(R)—N-(3,3-difluoro-1-methylpiperidin-4-yl)-6-fluoro-5-(1-(2-fluoroethyl)-1H-benzo[d][1,2,3]triazol-6-yl)-4-methoxypyrrolo[2,1-f][1,2,4]triazin-2-amine [710];
(R)—N-(3,3-difluoro-1-(methyl-d₃)piperidin-4-yl)-6-fluoro-5-(1-(2-fluoroethyl)-1H-benzo[d][1,2,3]triazol-6-yl)-4-methoxypyrrolo[2,1-f][1,2,4]triazin-2-amine [711];
(R)—N-(3,3-difluoro-1-(2-methoxyethyl)piperidin-4-yl)-6-fluoro-5-(1-(2-fluoroethyl)-1H-benzo[d][1,2,3]triazol-6-yl)-4-methoxypyrrolo[2,1-f][1,2,4]triazin-2-amine [712];
(R)-1-(3,3-difluoro-4-((6-fluoro-5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-one [713];
(R)-1-(3,3-difluoro-4-((6-fluoro-5-(1-(2-fluoroethyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [714];
(R)-1-(3,3-difluoro-4-((6-fluoro-5-(1-(2-fluoroethyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [715];
(R)—N-(3,3-difluoro-1-(methylsulfonyl)piperidin-4-yl)-6-fluoro-5-(1-(2-fluoroethyl)-1H-benzo[d][1,2,3]triazol-6-yl)-4-methoxypyrrolo[2,1-f][1,2,4]triazin-2-amine [716];
(R)—N-(3,3-difluoro-1-(oxetan-3-yl)piperidin-4-yl)-6-fluoro-5-(1-(2-fluoroethyl)-1H-benzo[d][1,2,3]triazol-6-yl)-4-methoxypyrrolo[2,1-f][1,2,4]triazin-2-amine [717];
(R)—N-(3,3-difluoro-1-(oxetan-3-yl-3-d)piperidin-4-yl)-6-fluoro-5-(1-(2-fluoroethyl)-1H-benzo[d][1,2,3]triazol-6-yl)-4-methoxypyrrolo[2,1-f][1,2,4]triazin-2-amine [718];
6-fluoro-5-(1-(2-fluoroethyl)-1H-benzo[d][1,2,3]triazol-6-yl)-4-methoxy-N-(2-oxaspiro[3.5]nonan-7-yl)pyrrolo[2,1-f][1,2,4]triazin-2-amine [719];
(1S,4S)-4-((6-fluoro-5-(1-(2-fluoroethyl)-1H-benzo[d][1,2,3]triazol-6-yl)-4-methoxypyrrolo[2,1-f][1,2,4]triazin-2-yl-7-d)amino)-1-methylcyclohexan-1-ol [720];
(1R,4R)-4-((6-fluoro-5-(1-(2-fluoroethyl)-1H-benzo[d][1,2,3]triazol-6-yl)-4-methoxypyrrolo[2,1-f][1,2,4]triazin-2-yl-7-d)amino)-1-methylcyclohexan-1-ol [721];
(R)—N-(4,4-difluoro-1-methylpyrrolidin-3-yl)-6-fluoro-5-(1-(2-fluoroethyl)-1H-benzo[d][1,2,3]triazol-6-yl)-4-methoxypyrrolo[2,1-f][1,2,4]triazin-7-d-2-amine [722];
(R)-1-(3,3-difluoro-4-((6-fluoro-5-(1-(2-fluoroethyl)-1H-benzo[d][1,2,3]triazol-6-yl)-4-methoxypyrrolo[2,1-f][1,2,4]triazin-2-yl-7-d)amino)pyrrolidin-1-yl)ethan-1-one [723];
(R)—N-(4,4-difluoro-1-(oxetan-3-yl)pyrrolidin-3-yl)-6-fluoro-5-(1-(2-fluoroethyl)-1H-benzo[d][1,2,3]triazol-6-yl)-4-methoxypyrrolo[2,1-f][1,2,4]triazin-7-d-2-amine [724];
6-fluoro-5-(1-(2-fluoroethyl)-1H-benzo[d][1,2,3]triazol-6-yl)-4-methoxy-N-(1-(oxetan-3-yl)piperidin-4-yl)pyrrolo[2,1-f][1,2,4]triazin-7-d-2-amine [725];
6-fluoro-N-((3S,4R)-3-fluoro-1-methylpiperidin-4-yl)-5-(1-(2-fluoroethyl)-1H-benzo[d][1,2,3]triazol-6-yl)-4-methoxypyrrolo[2,1-f][1,2,4]triazin-7-d-2-amine [726];
6-fluoro-N-((3R,4S)-3-fluoro-1-methylpiperidin-4-yl)-5-(1-(2-fluoroethyl)-1H-benzo[d][1,2,3]triazol-6-yl)-4-methoxypyrrolo[2,1-f][1,2,4]triazin-7-d-2-amine [727];
1-((3R,4S)-3-fluoro-4-((6-fluoro-5-(1-(2-fluoroethyl)-1H-benzo[d][1,2,3]triazol-6-yl)-4-methoxypyrrolo[2,1-f][1,2,4]triazin-2-yl-7-d)amino)piperidin-1-yl)ethan-1-one [728];
1-((3R,4S)-3-fluoro-4-((6-fluoro-5-(1-(2-fluoroethyl)-1H-benzo[d][1,2,3]triazol-6-yl)-4-methoxypyrrolo[2,1-f][1,2,4]triazin-2-yl-7-d)amino)piperidin-1-yl)ethan-1-one-2,2,2-d₃ [729];
1-((3R,4S)-3-fluoro-4-((6-fluoro-5-(1-(2-fluoroethyl)-1H-benzo[d][1,2,3]triazol-6-yl)-4-methoxypyrrolo[2,1-f][1,2,4]triazin-2-yl-7-d)amino)piperidin-1-yl)-2-hydroxyethan-1-one [730];
6-fluoro-N-((3S,4R)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-7-d-2-amine [731];
6-fluoro-N-((3R,4R)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-7-d-2-amine [732];
6-fluoro-N-((3S,4S)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-7-d-2-amine [733];
6-fluoro-N-((3R,4S)-3-fluoro-1-(oxetan-3-yl)piperidin-4-yl)-5-(1-(2-fluoroethyl)-1H-benzo[d][1,2,3]triazol-6-yl)-4-methoxypyrrolo[2,1-f][1,2,4]triazin-7-d-2-amine [734];
(R)—N-(3,3-difluoro-1-methylpiperidin-4-yl)-6-fluoro-5-(1-(2-fluoroethyl)-1H-benzo[d][1,2,3]triazol-6-yl)-4-methoxypyrrolo[2,1-f][1,2,4]triazin-7-d-2-amine [735];
(R)—N-(3,3-difluoro-1-(methyl-d₃)piperidin-4-yl)-6-fluoro-5-(1-(2-fluoroethyl)-1H-benzo[d][1,2,3]triazol-6-yl)-4-methoxypyrrolo[2,1-f][1,2,4]triazin-7-d-2-amine [736];
(R)-1-(3,3-difluoro-4-((6-fluoro-5-(1-(2-fluoroethyl)-1H-benzo[d][1,2,3]triazol-6-yl)-4-methoxypyrrolo[2,1-f][1,2,4]triazin-2-yl-7-d)amino)piperidin-1-yl)ethan-1-one [737];
(R)-1-(3,3-difluoro-4-((6-fluoro-5-(1-(2-fluoroethyl)-1H-benzo[d][1,2,3]triazol-6-yl)-4-methoxypyrrolo[2,1-f][1,2,4]triazin-2-yl-7-d)amino)piperidin-1-yl)ethan-1-one-2,2,2-d₃ [738];
(R)-1-(3,3-difluoro-4-((6-fluoro-5-(1-(2-fluoroethyl)-1H-benzo[d][1,2,3]triazol-6-yl)-4-methoxypyrrolo[2,1-f][1,2,4]triazin-2-yl-7-d)amino)piperidin-1-yl)-2-hydroxyethan-1-one [739];
(R)—N-(3,3-difluoro-1-(methylsulfonyl)piperidin-4-yl)-6-fluoro-5-(1-(2-fluoroethyl)-1H-benzo[d][1,2,3]triazol-6-yl)-4-methoxypyrrolo[2,1-f][1,2,4]triazin-7-d-2-amine [740];
(R)—N-(3,3-difluoro-1-(oxetan-3-yl)piperidin-4-yl)-6-fluoro-5-(1-(2-fluoroethyl)-1H-benzo[d][1,2,3]triazol-6-yl)-4-methoxypyrrolo[2,1-f][1,2,4]triazin-7-d-2-amine [741];
6-fluoro-5-(1-(2-fluoroethyl)-1H-benzo[d][1,2,3]triazol-6-yl)-4-methoxy-N-(2-oxaspiro[3.5]nonan-7-yl)pyrrolo[2,1-f][1,2,4]triazin-7-d-2-amine [742];
(1S,4S)-4-((6-fluoro-5-(1-(2-fluoroethyl)-1H-benzo[d][1,2,3]triazol-6-yl)-4-(methoxy-d₃)pyrrolo[2,1-f][1,2,4]triazin-2-yl)amino)-1-methylcyclohexan-1-ol [743];
(1R,4R)-4-((6-fluoro-5-(1-(2-fluoroethyl)-1H-benzo[d][1,2,3]triazol-6-yl)-4-(methoxy-d₃)pyrrolo[2,1-f][1,2,4]triazin-2-yl)amino)-1-methylcyclohexan-1-ol [744];
(R)—N-(4,4-difluoro-1-methylpyrrolidin-3-yl)-6-fluoro-5-(1-(2-fluoroethyl)-1H-benzo[d][1,2,3]triazol-6-yl)-4-(methoxy-d₃)pyrrolo[2,1-f][1,2,4]triazin-2-amine [745];
(R)-1-(3,3-difluoro-4-((6-fluoro-5-(1-(2-fluoroethyl)-1H-benzo[d][1,2,3]triazol-6-yl)-4-(methoxy-d₃)pyrrolo[2,1-f][1,2,4]triazin-2-yl)amino)pyrrolidin-1-yl)ethan-1-one [746];
(R)—N-(4,4-difluoro-1-(oxetan-3-yl)pyrrolidin-3-yl)-6-fluoro-5-(1-(2-fluoroethyl)-1H-benzo[d][1,2,3]triazol-6-yl)-4-(methoxy-d₃)pyrrolo[2,1-f][1,2,4]triazin-2-amine [747];
6-fluoro-5-(1-(2-fluoroethyl)-1H-benzo[d][1,2,3]triazol-6-yl)-4-(methoxy-d)-N-(1-(oxetan-3-yl)piperidin-4-yl)pyrrolo[2,1-f][1,2,4]triazin-2-amine [748];
6-fluoro-N-((3S,4R)-3-fluoro-1-methylpiperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [749];
6-fluoro-N-((3R,4S)-3-fluoro-1-methylpiperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [750];
1-((3R,4S)-3-fluoro-4-((6-fluoro-5-(1-(2-fluoroethyl)-1H-benzo[d][1,2,3]triazol-6-yl)-4-(methoxy-d₃)pyrrolo[2,1-f][1,2,4]triazin-2-yl)amino)piperidin-1-yl)ethan-1-one [751];
1-((3R,4S)-3-fluoro-4-((6-fluoro-5-(1-(2-fluoroethyl)-1H-benzo[d][1,2,3]triazol-6-yl)-4-(methoxy-d₃)pyrrolo[2,1-f][1,2,4]triazin-2-yl)amino)piperidin-1-yl)ethan-1-one-2,2,2-d₃ [752];
1-((3R,4S)-3-fluoro-4-((6-fluoro-5-(1-(2-fluoroethyl)-1H-benzo[d][1,2,3]triazol-6-yl)-4-(methoxy-d₃)pyrrolo[2,1-f][1,2,4]triazin-2-yl)amino)piperidin-1-yl)-2-hydroxyethan-1-one [753];
6-fluoro-N-((3S,4R)-3-fluoro-1-(oxetan-3-yl)piperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [754];
6-fluoro-N-((3R,4R)-3-fluoro-1-(oxetan-3-yl)piperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [755];
6-fluoro-N-((3S,4S)-3-fluoro-1-(oxetan-3-yl)piperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [756];
6-fluoro-N-((3R,4S)-3-fluoro-1-(oxetan-3-yl)piperidin-4-yl)-5-(1-(2-fluoroethyl)-1H-benzo[d][1,2,3]triazol-6-yl)-4-(methoxy-d₃)pyrrolo[2,1-f][1,2,4]triazin-2-amine [757];
(R)—N-(3,3-difluoro-1-methylpiperidin-4-yl)-6-fluoro-5-(1-(2-fluoroethyl)-1H-benzo[d][1,2,3]triazol-6-yl)-4-(methoxy-d₃)pyrrolo[2,1-f][1,2,4]triazin-2-amine [758];
(R)—N-(3,3-difluoro-1-(methyl-d₃)piperidin-4-yl)-6-fluoro-5-(1-(2-fluoroethyl)-1H-benzo[d][1,2,3]triazol-6-yl)-4-(methoxy-d₃)pyrrolo[2,1-f][1,2,4]triazin-2-amine [759];
(R)-1-(3,3-difluoro-4-((6-fluoro-5-(1-(2-fluoroethyl)-1H-benzo[d][1,2,3]triazol-6-yl)-4-(methoxy-d₃)pyrrolo[2,1-f][1,2,4]triazin-2-yl)amino)piperidin-1-yl)ethan-1-one [760];
(R)-1-(3,3-difluoro-4-((6-fluoro-5-(1-(2-fluoroethyl)-1H-benzo[d][1,2,3]triazol-6-yl)-4-(methoxy-d₃)pyrrolo[2,1-f][1,2,4]triazin-2-yl)amino)piperidin-1-yl)ethan-1-one-2,2,2-d₃ [761];
(R)-1-(3,3-difluoro-4-((6-fluoro-5-(1-(2-fluoroethyl)-1H-benzo[d][1,2,3]triazol-6-yl)-4-(methoxy-d₃)pyrrolo[2,1-f][1,2,4]triazin-2-yl)amino)piperidin-1-yl)-2-hydroxyethan-1-one [762];
(R)—N-(3,3-difluoro-1-(methylsulfonyl)piperidin-4-yl)-6-fluoro-5-(1-(2-fluoroethyl)-1H-benzo[d][1,2,3]triazol-6-yl)-4-(methoxy-d₃)pyrrolo[2,1-f][1,2,4]triazin-2-amine [763];
(R)—N-(3,3-difluoro-1-(oxetan-3-yl)piperidin-4-yl)-6-fluoro-5-(1-(2-fluoroethyl)-1H-benzo[d][1,2,3]triazol-6-yl)-4-(methoxy-d₃)pyrrolo[2,1-f][1,2,4]triazin-2-amine [764];
6-fluoro-5-(1-(2-fluoroethyl)-1H-benzo[d][1,2,3]triazol-6-yl)-4-(methoxy-d₃)—N-(2-oxaspiro[3.5]nonan-7-yl)pyrrolo[2,1-f][1,2,4]triazin-2-amine [765];
2-((5-(1-(2,2-difluoroethyl)-1H-benzo[d][1,2,3]triazol-6-yl)-4-methoxypyrrolo[2,1-f][1,2,4]triazin-2-yl)amino)-1-morpholinoethan-1-one [766];
(R)—N-(4,4-difluoro-1-(oxetan-3-yl)pyrrolidin-3-yl)-5-(1-(2,2-difluoroethyl)-1H-benzo[d][1,2,3]triazol-6-yl)-4-methoxypyrrolo[2,1-f][1,2,4]triazin-2-amine [767];
5-(1-(2,2-difluoroethyl)-1H-benzo[d][1,2,3]triazol-6-yl)-N-((3S,4R)-3-fluoro-1-(2-methoxyethyl)piperidin-4-yl)-4-methoxypyrrolo[2,1-f][1,2,4]triazin-2-amine [768];
5-(1-(2,2-difluoroethyl)-1H-benzo[d][1,2,3]triazol-6-yl)-N-((3R,4R)-3-fluoro-1-(2-methoxyethyl)piperidin-4-yl)-4-methoxypyrrolo[2,1-f][1,2,4]triazin-2-amine [769];
5-(1-(2,2-difluoroethyl)-1H-benzo[d][1,2,3]triazol-6-yl)-N-((3S,4S)-3-fluoro-1-(2-methoxyethyl)piperidin-4-yl)-4-methoxypyrrolo[2,1-f][1,2,4]triazin-2-amine [770];
5-(1-(2,2-difluoroethyl)-1H-benzo[d][1,2,3]triazol-6-yl)-N-((3R,4S)-3-fluoro-1-(2-methoxyethyl)piperidin-4-yl)-4-methoxypyrrolo[2,1-f][1,2,4]triazin-2-amine [771];
2-((3R,4S)-4-((5-(1-(2,2-difluoroethyl)-1H-benzo[d][1,2,3]triazol-6-yl)-4-methoxypyrrolo[2,1-f][1,2,4]triazin-2-yl)amino)-3-fluoropiperidin-1-yl)-N,N-dimethylacetamide [772];
1-(azetidin-1-yl)-2-((3R,4S)-4-((5-(1-(2,2-difluoroethyl)-1H-benzo[d][1,2,3]triazol-6-yl)-4-methoxypyrrolo[2,1-f][1,2,4]triazin-2-yl)amino)-3-fluoropiperidin-1-yl)ethan-1-one [773];
1-((3R,4S)-4-((5-(1-(2,2-difluoroethyl)-1H-benzo[d][1,2,3]triazol-6-yl)-4-methoxypyrrolo[2,1-f][1,2,4]triazin-2-yl)amino)-3-fluoropiperidin-1-yl)-2-hydroxyethan-1-one [774];
1-((3R,4S)-4-((5-(1-(2,2-difluoroethyl)-1H-benzo[d][1,2,3]triazol-6-yl)-4-methoxypyrrolo[2,1-f][1,2,4]triazin-2-yl)amino)-3-fluoropiperidin-1-yl)-2-(pyrrolidin-1-yl)ethan-1-one [775];
(3R,4S)-4-((5-(1-(2,2-difluoroethyl)-1H-benzo[d][1,2,3]triazol-6-yl)-4-methoxypyrrolo[2,1-f][1,2,4]triazin-2-yl)amino)-3-fluoro-N-methylpiperidine-1-carboxamide [776];
(3R,4S)-4-((5-(1-(2,2-difluoroethyl)-1H-benzo[d][1,2,3]triazol-6-yl)-4-methoxypyrrolo[2,1-f][1,2,4]triazin-2-yl)amino)-3-fluoro-N,N-dimethylpiperidine-1-carboxamide [777];
((3R,4S)-4-((5-(1-(2,2-difluoroethyl)-1H-benzo[d][1,2,3]triazol-6-yl)-4-methoxypyrrolo[2,1-f][1,2,4]triazin-2-yl)amino)-3-fluoropiperidin-1-yl)(pyrrolidin-1-yl)methanone [778];
5-(1-(2,2-difluoroethyl)-1H-benzo[d][1,2,3]triazol-6-yl)-N-((3R,4R)-3-fluoro-1-(oxetan-3-yl)piperidin-4-yl)-4-methoxypyrrolo[2,1-f][1,2,4]triazin-2-amine [779];
5-(1-(2,2-difluoroethyl)-1H-benzo[d][1,2,3]triazol-6-yl)-N-((3S,4S)-3-fluoro-1-(oxetan-3-yl)piperidin-4-yl)-4-methoxypyrrolo[2,1-f][1,2,4]triazin-2-amine [780];
5-(1-(2,2-difluoroethyl)-1H-benzo[d][1,2,3]triazol-6-yl)-N-((3S,4R)-3-fluoro-1-(oxetan-3-yl-3-d)piperidin-4-yl)-4-methoxypyrrolo[2,1-f][1,2,4]triazin-2-amine [781];
5-(1-(2,2-difluoroethyl)-1H-benzo[d][1,2,3]triazol-6-yl)-N-((3R,4R)-3-fluoro-1-(oxetan-3-yl-3-d)piperidin-4-yl)-4-methoxypyrrolo[2,1-f][1,2,4]triazin-2-amine [782];
5-(1-(2,2-difluoroethyl)-1H-benzo[d][1,2,3]triazol-6-yl)-N-((3S,4S)-3-fluoro-1-(oxetan-3-yl-3-d)piperidin-4-yl)-4-methoxypyrrolo[2,1-f][1,2,4]triazin-2-amine [783];
5-(1-(2,2-difluoroethyl)-1H-benzo[d][1,2,3]triazol-6-yl)-N-((3R,4S)-3-fluoro-1-(oxetan-3-yl-3-d)piperidin-4-yl)-4-methoxypyrrolo[2,1-f][1,2,4]triazin-2-amine [784];
(S)—N-(3,3-difluoro-1-methylpiperidin-4-yl)-5-(1-(2,2-difluoroethyl)-1H-benzo[d][1,2,3]triazol-6-yl)-4-methoxypyrrolo[2,1-f][1,2,4]triazin-2-amine [785];
(R)—N-(3,3-difluoro-1-(2-methoxyethyl)piperidin-4-yl)-5-(1-(2,2-difluoroethyl)-1H-benzo[d][1,2,3]triazol-6-yl)-4-methoxypyrrolo[2,1-f][1,2,4]triazin-2-amine [786];
(R)-1-(azetidin-1-yl)-2-(4-((5-(1-(2,2-difluoroethyl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one [787];
(S)-1-(4-((5-(1-(2,2-difluoroethyl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one [788];
(R)-1-(4-((5-(1-(2,2-difluoroethyl)-1H-benzo[d][1,2,3]triazol-6-yl)-4-methoxypyrrolo[2,1-f][1,2,4]triazin-2-yl)amino)-3,3-difluoropiperidin-1-yl)-2-hydroxyethan-1-one [789];
(R)-1-(4-((5-(1-(2,2-difluoroethyl)-1H-benzo[d][1,2,3]triazol-6-yl)-4-methoxypyrrolo[2,1-f][1,2,4]triazin-2-yl)amino)-3,3-difluoropiperidin-1-yl)-2-(dimethylamino)ethan-1-one [790];
(R)-(4-((5-(1-(2,2-difluoroethyl)-1H-benzo[d][1,2,3]triazol-6-yl)-4-methoxypyrrolo[2,1-f][1,2,4]triazin-2-yl)amino)-3,3-difluoropiperidin-1-yl)(pyrrolidin-1-yl)methanone [791];
(R)—N-(3,3-difluoro-1-(oxetan-3-yl-3-d)piperidin-4-yl)-5-(1-(2,2-difluoroethyl)-1H-benzo[d][1,2,3]triazol-6-yl)-4-methoxypyrrolo[2,1-f][1,2,4]triazin-2-amine [792];
(S)-4-((5-(1-(2,2-difluoroethyl)-1H-benzo[d][1,2,3]triazol-6-yl)-4-methoxypyrrolo[2,1-f][1,2,4]triazin-2-yl)amino)-1-methylpiperidin-2-one [793];
(R)—N-(4,4-difluoro-1-methylpyrrolidin-3-yl)-5-(1-(2,2-difluoroethyl)-1H-benzo[d][1,2,3]triazol-6-yl)-4-methoxypyrrolo[2,1-f][1,2,4]triazin-7-d-2-amine [794];
(R)-1-(4-((5-(1-(2,2-difluoroethyl)-1H-benzo[d][1,2,3]triazol-6-yl)-4-methoxypyrrolo[2,1-f][1,2,4]triazin-2-yl-7-d)amino)-3,3-difluoropyrrolidin-1-yl)ethan-1-one [795];
(R)—N-(4,4-difluoro-1-(oxetan-3-yl)pyrrolidin-3-yl)-5-(1-(2,2-difluoroethyl)-1H-benzo[d][1,2,3]triazol-6-yl)-4-methoxypyrrolo[2,1-f][1,2,4]triazin-7-d-2-amine [796];
1-((3R,4S)-4-((5-(1-(2,2-difluoroethyl)-1H-benzo[d][1,2,3]triazol-6-yl)-4-methoxypyrrolo[2,1-f][1,2,4]triazin-2-yl-7-d)amino)-3-fluoropiperidin-1-yl)ethan-1-one-2,2,2-d₃ [797];
1-((3R,4S)-4-((5-(1-(2,2-difluoroethyl)-1H-benzo[d][1,2,3]triazol-6-yl)-4-methoxypyrrolo[2,1-f][1,2,4]triazin-2-yl-7-d)amino)-3-fluoropiperidin-1-yl)-2-hydroxyethan-1-one [798];
5-(1-(2,2-difluoroethyl)-1H-benzo[d][1,2,3]triazol-6-yl)-N-((3S,4R)-3-fluoro-1-(oxetan-3-yl)piperidin-4-yl)-4-methoxypyrrolo[2,1-f][1,2,4]triazin-7-d-2-amine [799]; and
5-(1-(2,2-difluoroethyl)-1H-benzo[d][1,2,3]triazol-6-yl)-N-((3R,4R)-3-fluoro-1-(oxetan-3-yl)piperidin-4-yl)-4-methoxypyrrolo[2,1-f][1,2,4]triazin-7-d-2-amine [800]; or a pharmaceutically acceptable salt thereof.

25. The compound of claim 1, wherein the compound of Formula I is selected from the group consisting of
5-(1-(2,2-difluoroethyl)-1H-benzo[d][1,2,3]triazol-6-yl)-N-((3S,4S)-3-fluoro-1-(oxetan-3-yl)piperidin-4-yl)-4-methoxypyrrolo[2,1-f][1,2,4]triazin-7-d-2-amine [801];
(R)—N-(3,3-difluoro-1-methylpiperidin-4-yl)-5-(1-(2,2-difluoroethyl)-1H-benzo[d][1,2,3]triazol-6-yl)-4-methoxypyrrolo[2,1-f][1,2,4]triazin-7-d-2-amine [802];
(R)—N-(3,3-difluoro-1-(methyl-d₃)piperidin-4-yl)-5-(1-(2,2-difluoroethyl)-1H-benzo[d][1,2,3]triazol-6-yl)-4-methoxypyrrolo[2,1-f][1,2,4]triazin-7-d-2-amine [803];
(R)-1-(4-((5-(1-(2,2-difluoroethyl)-1H-benzo[d][1,2,3]triazol-6-yl)-4-methoxypyrrolo[2,1-f][1,2,4]triazin-2-yl-7-d)amino)-3,3-difluoropiperidin-1-yl)ethan-1-one-2,2,2-d₃ [804];
(R)-1-(4-((5-(1-(2,2-difluoroethyl)-1H-benzo[d][1,2,3]triazol-6-yl)-4-methoxypyrrolo[2,1-f][1,2,4]triazin-2-yl-7-d)amino)-3,3-difluoropiperidin-1-yl)-2-hydroxyethan-1-one [805];
(R)—N-(3,3-difluoro-1-(methylsulfonyl)piperidin-4-yl)-5-(1-(2,2-difluoroethyl)-1H-benzo[d][1,2,3]triazol-6-yl)-4-methoxypyrrolo[2,1-f][1,2,4]triazin-7-d-2-amine [806];
5-(1-(2,2-difluoroethyl)-1H-benzo[d][1,2,3]triazol-6-yl)-4-methoxy-N-(2-oxaspiro[3.5]nonan-7-yl)pyrrolo[2,1-f][1,2,4]triazin-7-d-2-amine [807];
(R)—N-(4,4-difluoro-1-methylpyrrolidin-3-yl)-5-(1-(2,2-difluoroethyl)-1H-benzo[d][1,2,3]triazol-6-yl)-4-(methoxy-d₃)pyrrolo[2,1-f][1,2,4]triazin-2-amine [808];
(R)-1-(4-((5-(1-(2,2-difluoroethyl)-1H-benzo[d][1,2,3]triazol-6-yl)-4-(methoxy-d₃)pyrrolo[2,1-f][1,2,4]triazin-2-yl)amino)-3,3-difluoropyrrolidin-1-yl)ethan-1-one [809];
(R)—N-(4,4-difluoro-1-(oxetan-3-yl)pyrrolidin-3-yl)-5-(1-(2,2-difluoroethyl)-1H-benzo[d][1,2,3]triazol-6-yl)-4-(methoxy-d₃)pyrrolo[2,1-f][1,2,4]triazin-2-amine [810];
1-((3R,4S)-4-((5-(1-(2,2-difluoroethyl)-1H-benzo[d][1,2,3]triazol-6-yl)-4-(methoxy-d₃)pyrrolo[2,1-f][1,2,4]triazin-2-yl)amino)-3-fluoropiperidin-1-yl)ethan-1-one-2,2,2-d₃ [811];
1-((3R,4S)-4-((5-(1-(2,2-difluoroethyl)-1H-benzo[d][1,2,3]triazol-6-yl)-4-(methoxy-d₃)pyrrolo[2,1-f][1,2,4]triazin-2-yl)amino)-3-fluoropiperidin-1-yl)-2-hydroxyethan-1-one [812];
5-(1-(2,2-difluoroethyl)-1H-benzo[d][1,2,3]triazol-6-yl)-N-((3S,4R)-3-fluoro-1-(oxetan-3-yl)piperidin-4-yl)-4-(methoxy-d₃)pyrrolo[2,1-f][1,2,4]triazin-2-amine [813];
5-(1-(2,2-difluoroethyl)-1H-benzo[d][1,2,3]triazol-6-yl)-N-((3R,4R)-3-fluoro-1-(oxetan-3-yl)piperidin-4-yl)-4-(methoxy-d₃)pyrrolo[2,1-f][1,2,4]triazin-2-amine [814];
5-(1-(2,2-difluoroethyl)-1H-benzo[d][1,2,3]triazol-6-yl)-N-((3S,4S)-3-fluoro-1-(oxetan-3-yl)piperidin-4-yl)-4-(methoxy-d₃)pyrrolo[2,1-f][1,2,4]triazin-2-amine [815];
(R)—N-(3,3-difluoro-1-methylpiperidin-4-yl)-5-(1-(2,2-difluoroethyl)-1H-benzo[d][1,2,3]triazol-6-yl)-4-(methoxy-d₃)pyrrolo[2,1-f][1,2,4]triazin-2-amine [816];
(R)—N-(3,3-difluoro-1-(methyl-d₃)piperidin-4-yl)-5-(1-(2,2-difluoroethyl)-1H-benzo[d][1,2,3]triazol-6-yl)-4-(methoxy-d₃)pyrrolo[2,1-f][1,2,4]triazin-2-amine [817];
(R)-1-(4-((5-(1-(2,2-difluoroethyl)-1H-benzo[d][1,2,3]triazol-6-yl)-4-(methoxy-d₃)pyrrolo[2,1-f][1,2,4]triazin-2-yl)amino)-3,3-difluoropiperidin-1-yl)ethan-1-one-2,2,2-d₃ [818];
(R)-1-(4-((5-(1-(2,2-difluoroethyl)-1H-benzo[d][1,2,3]triazol-6-yl)-4-(methoxy-d₃)pyrrolo[2,1-f][1,2,4]triazin-2-yl)amino)-3,3-difluoropiperidin-1-yl)-2-hydroxyethan-1-one [819];
(R)—N-(3,3-difluoro-1-(methylsulfonyl)piperidin-4-yl)-5-(1-(2,2-difluoroethyl)-1H-benzo[d][1,2,3]triazol-6-yl)-4-(methoxy-d₃)pyrrolo[2,1-f][1,2,4]triazin-2-amine [820];
5-(1-(2,2-difluoroethyl)-1H-benzo[d][1,2,3]triazol-6-yl)-4-(methoxy-d)-N-(2-oxaspiro[3.5]nonan-7-yl)pyrrolo[2,1-f][1,2,4]triazin-2-amine [821];
3-((5-(1-(2,2-difluoroethyl)-1H-benzo[d][1,2,3]triazol-6-yl)-6-fluoro-4-methoxypyrrolo[2,1-f][1,2,4]triazin-2-yl)amino)-2,2-dimethylpropanenitrile [822];
5-(1-(2,2-difluoroethyl)-1H-benzo[d][1,2,3]triazol-6-yl)-6-fluoro-N-((3R,4S)-4-fluoro-1-(oxetan-3-yl)pyrrolidin-3-yl)-4-methoxypyrrolo[2,1-f][1,2,4]triazin-2-amine [823];
(R)—N-(4,4-difluoro-1-methylpyrrolidin-3-yl)-5-(1-(2,2-difluoroethyl)-1H-benzo[d][1,2,3]triazol-6-yl)-6-fluoro-4-methoxypyrrolo[2,1-f][1,2,4]triazin-2-amine [824];
(R)-1-(4-((5-(1-(2,2-difluoroethyl)-1H-benzo[d][1,2,3]triazol-6-yl)-6-fluoro-4-methoxypyrrolo[2,1-f][1,2,4]triazin-2-yl)amino)-3,3-difluoropyrrolidin-1-yl)ethan-1-one [825];
(R)—N-(4,4-difluoro-1-(oxetan-3-yl)pyrrolidin-3-yl)-5-(1-(2,2-difluoroethyl)-1H-benzo[d][1,2,3]triazol-6-yl)-6-fluoro-4-methoxypyrrolo[2,1-f][1,2,4]triazin-2-amine [826];
5-(1-(2,2-difluoroethyl)-1H-benzo[d][1,2,3]triazol-6-yl)-6-fluoro-N-((3S,4R)-3-fluoro-1-methylpiperidin-4-yl)-4-methoxypyrrolo[2,1-f][1,2,4]triazin-2-amine [827];
5-(1-(2,2-difluoroethyl)-1H-benzo[d][1,2,3]triazol-6-yl)-6-fluoro-N-((3S,4R)-3-fluoro-1-(2-methoxyethyl)piperidin-4-yl)-4-methoxypyrrolo[2,1-f][1,2,4]triazin-2-amine [828];
5-(1-(2,2-difluoroethyl)-1H-benzo[d][1,2,3]triazol-6-yl)-6-fluoro-N-((3R,4R)-3-fluoro-1-(2-methoxyethyl)piperidin-4-yl)-4-methoxypyrrolo[2,1-f][1,2,4]triazin-2-amine [829];
5-(1-(2,2-difluoroethyl)-1H-benzo[d][1,2,3]triazol-6-yl)-6-fluoro-N-((3S,4S)-3-fluoro-1-(2-methoxyethyl)piperidin-4-yl)-4-methoxypyrrolo[2,1-f][1,2,4]triazin-2-amine [830];
5-(1-(2,2-difluoroethyl)-1H-benzo[d][1,2,3]triazol-6-yl)-6-fluoro-N-((3R,4S)-3-fluoro-1-(2-methoxyethyl)piperidin-4-yl)-4-methoxypyrrolo[2,1-f][1,2,4]triazin-2-amine [831];
1-((3R,4S)-4-((5-(1-(2,2-difluoroethyl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one-2,2,2-d₃ [832];
1-((3R,4S)-4-((5-(1-(2,2-difluoroethyl)-1H-benzo[d][1,2,3]triazol-6-yl)-6-fluoro-4-methoxypyrrolo[2,1-f][1,2,4]triazin-2-yl)amino)-3-fluoropiperidin-1-yl)-2-hydroxyethan-1-one [833];
5-(1-(2,2-difluoroethyl)-1H-benzo[d][1,2,3]triazol-6-yl)-6-fluoro-N-((3S,4R)-3-fluoro-1-(oxetan-3-yl)piperidin-4-yl)-4-methoxypyrrolo[2,1-f][1,2,4]triazin-2-amine [834];
5-(1-(2,2-difluoroethyl)-1H-benzo[d][1,2,3]triazol-6-yl)-6-fluoro-N-((3R,4R)-3-fluoro-1-(oxetan-3-yl)piperidin-4-yl)-4-methoxypyrrolo[2,1-f][1,2,4]triazin-2-amine [835];
5-(1-(2,2-difluoroethyl)-1H-benzo[d][1,2,3]triazol-6-yl)-6-fluoro-N-((3S,4S)-3-fluoro-1-(oxetan-3-yl)piperidin-4-yl)-4-methoxypyrrolo[2,1-f][1,2,4]triazin-2-amine [836];
5-(1-(2,2-difluoroethyl)-1H-benzo[d][1,2,3]triazol-6-yl)-6-fluoro-N-((3S,4R)-3-fluoro-1-(oxetan-3-yl-3-d)piperidin-4-yl)-4-methoxypyrrolo[2,1-f][1,2,4]triazin-2-amine [837];
5-(1-(2,2-difluoroethyl)-1H-benzo[d][1,2,3]triazol-6-yl)-6-fluoro-N-((3R,4R)-3-fluoro-1-(oxetan-3-yl-3-d)piperidin-4-yl)-4-methoxypyrrolo[2,1-f][1,2,4]triazin-2-amine [838];
5-(1-(2,2-difluoroethyl)-1H-benzo[d][1,2,3]triazol-6-yl)-6-fluoro-N-((3S,4S)-3-fluoro-1-(oxetan-3-yl-3-d)piperidin-4-yl)-4-methoxypyrrolo[2,1-f][1,2,4]triazin-2-amine [839];
5-(1-(2,2-difluoroethyl)-1H-benzo[d][1,2,3]triazol-6-yl)-6-fluoro-N-((3R,4S)-3-fluoro-1-(oxetan-3-yl-3-d)piperidin-4-yl)-4-methoxypyrrolo[2,1-f][1,2,4]triazin-2-amine [840];
(R)—N-(3,3-difluoro-1-methylpiperidin-4-yl)-5-(1-(2,2-difluoroethyl)-1H-benzo[d][1,2,3]triazol-6-yl)-6-fluoro-4-methoxypyrrolo[2,1-f][1,2,4]triazin-2-amine [841];
(R)—N-(3,3-difluoro-1-(methyl-d₃)piperidin-4-yl)-5-(1-(2,2-difluoroethyl)-1H-benzo[d][1,2,3]triazol-6-yl)-6-fluoro-4-methoxypyrrolo[2,1-f][1,2,4]triazin-2-amine [842];
(R)—N-(3,3-difluoro-1-(2-methoxyethyl)piperidin-4-yl)-5-(1-(2,2-difluoroethyl)-1H-benzo[d][1,2,3]triazol-6-yl)-6-fluoro-4-methoxypyrrolo[2,1-f][1,2,4]triazin-2-amine [843];
(R)-1-(4-((5-(1-(2,2-difluoroethyl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)ethan-1-one-2,2,2-d₃ [844];
(R)-1-(4-((5-(1-(2,2-difluoroethyl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)-2-hydroxyethan-1-one [845];
(R)—N-(3,3-difluoro-1-(methylsulfonyl)piperidin-4-yl)-5-(1-(2,2-difluoroethyl)-1H-benzo[d][1,2,3]triazol-6-yl)-6-fluoro-4-methoxypyrrolo[2,1-f][1,2,4]triazin-2-amine [846];
(R)—N-(3,3-difluoro-1-(oxetan-3-yl-3-d)piperidin-4-yl)-5-(1-(2,2-difluoroethyl)-1H-benzo[d][1,2,3]triazol-6-yl)-6-fluoro-4-methoxypyrrolo[2,1-f][1,2,4]triazin-2-amine [847];
5-(1-(2,2-difluoroethyl)-1H-benzo[d][1,2,3]triazol-6-yl)-6-fluoro-4-methoxy-N-(2-oxaspiro[3.5]nonan-7-yl)pyrrolo[2,1-f][1,2,4]triazin-2-amine [848];
(R)—N-(4,4-difluoro-1-methylpyrrolidin-3-yl)-5-(1-(2,2-difluoroethyl)-1H-benzo[d][1,2,3]triazol-6-yl)-6-fluoro-4-methoxypyrrolo[2,1-f][1,2,4]triazin-7-d-2-amine [849];
(R)-1-(4-((5-(1-(2,2-difluoroethyl)-1H-benzo[d][1,2,3]triazol-6-yl)-6-fluoro-4-methoxypyrrolo[2,1-f][1,2,4]triazin-2-yl-7-d)amino)-3,3-difluoropyrrolidin-1-yl)ethan-1-one [850];
(R)—N-(4,4-difluoro-1-(oxetan-3-yl)pyrrolidin-3-yl)-5-(1-(2,2-difluoroethyl)-1H-benzo[d][1,2,3]triazol-6-yl)-6-fluoro-4-methoxypyrrolo[2,1-f][1,2,4]triazin-7-d-2-amine [851];
1-((3R,4S)-4-((5-(1-(2,2-difluoroethyl)-1H-benzo[d][1,2,3]triazol-6-yl)-6-fluoro-4-methoxypyrrolo[2,1-f][1,2,4]triazin-2-yl-7-d)amino)-3-fluoropiperidin-1-yl)ethan-1-one-2,2,2-d₃ [852];
1-((3R,4S)-4-((5-(1-(2,2-difluoroethyl)-1H-benzo[d][1,2,3]triazol-6-yl)-6-fluoro-4-methoxypyrrolo[2,1-f][1,2,4]triazin-2-yl-7-d)amino)-3-fluoropiperidin-1-yl)-2-hydroxyethan-1-one [853];
5-(1-(2,2-difluoroethyl)-1H-benzo[d][1,2,3]triazol-6-yl)-6-fluoro-N-((3S,4R)-3-fluoro-1-(oxetan-3-yl)piperidin-4-yl)-4-methoxypyrrolo[2,1-f][1,2,4]triazin-7-d-2-amine [854];
5-(1-(2,2-difluoroethyl)-1H-benzo[d][1,2,3]triazol-6-yl)-6-fluoro-N-((3R,4R)-3-fluoro-1-(oxetan-3-yl)piperidin-4-yl)-4-methoxypyrrolo[2,1-f][1,2,4]triazin-7-d-2-amine [855];
5-(1-(2,2-difluoroethyl)-1H-benzo[d][1,2,3]triazol-6-yl)-6-fluoro-N-((3S,4S)-3-fluoro-1-(oxetan-3-yl)piperidin-4-yl)-4-methoxypyrrolo[2,1-f][1,2,4]triazin-7-d-2-amine [856];
(R)—N-(3,3-difluoro-1-methylpiperidin-4-yl)-5-(1-(2,2-difluoroethyl)-1H-benzo[d][1,2,3]triazol-6-yl)-6-fluoro-4-methoxypyrrolo[2,1-f][1,2,4]triazin-7-d-2-amine [857];
(R)—N-(3,3-difluoro-1-(methyl-d₃)piperidin-4-yl)-5-(1-(2,2-difluoroethyl)-1H-benzo[d][1,2,3]triazol-6-yl)-6-fluoro-4-methoxypyrrolo[2,1-f][1,2,4]triazin-7-d-2-amine [858];
(R)-1-(4-((5-(1-(2,2-difluoroethyl)-1H-benzo[d][1,2,3]triazol-6-yl)-6-fluoro-4-methoxypyrrolo[2,1-f][1,2,4]triazin-2-yl-7-d)amino)-3,3-difluoropiperidin-1-yl)ethan-1-one-2,2,2-d₃ [859];
(R)-1-(4-((5-(1-(2,2-difluoroethyl)-1H-benzo[d][1,2,3]triazol-6-yl)-6-fluoro-4-methoxypyrrolo[2,1-f][1,2,4]triazin-2-yl-7-d)amino)-3,3-difluoropiperidin-1-yl)-2-hydroxyethan-1-one [860];
(R)—N-(3,3-difluoro-1-(methylsulfonyl)piperidin-4-yl)-5-(1-(2,2-difluoroethyl)-1H-benzo[d][1,2,3]triazol-6-yl)-6-fluoro-4-methoxypyrrolo[2,1-f][1,2,4]triazin-7-d-2-amine [861];
5-(1-(2,2-difluoroethyl)-1H-benzo[d][1,2,3]triazol-6-yl)-6-fluoro-4-methoxy-N-(2-oxaspiro[3.5]nonan-7-yl)pyrrolo[2,1-f][1,2,4]triazin-7-d-2-amine [862];
(1s,4s)-4-((5-(1-(2,2-difluoroethyl)-1H-benzo[d][1,2,3]triazol-6-yl)-6-fluoro-4-(methoxy-d₃)pyrrolo[2,1-f][1,2,4]triazin-2-yl)amino)-1-methylcyclohexan-1-ol [863];
(1r,4r)-4-((5-(1-(2,2-difluoroethyl)-1H-benzo[d][1,2,3]triazol-6-yl)-6-fluoro-4-(methoxy-d₃)pyrrolo[2,1-f][1,2,4]triazin-2-yl)amino)-1-methylcyclohexan-1-ol [864];
(R)—N-(4,4-difluoro-1-methylpyrrolidin-3-yl)-5-(1-(2,2-difluoroethyl)-1H-benzo[d][1,2,3]triazol-6-yl)-6-fluoro-4-(methoxy-d₃)pyrrolo[2,1-f][1,2,4]triazin-2-amine [865];
(R)-1-(4-((5-(1-(2,2-difluoroethyl)-1H-benzo[d][1,2,3]triazol-6-yl)-6-fluoro-4-(methoxy-d₃)pyrrolo[2,1-f][1,2,4]triazin-2-yl)amino)-3,3-difluoropyrrolidin-1-yl)ethan-1-one [866];
(R)—N-(4,4-difluoro-1-(oxetan-3-yl)pyrrolidin-3-yl)-5-(1-(2,2-difluoroethyl)-1H-benzo[d][1,2,3]triazol-6-yl)-6-fluoro-4-(methoxy-d₃)pyrrolo[2,1-f][1,2,4]triazin-2-amine [867];
5-(1-(2,2-difluoroethyl)-1H-benzo[d][1,2,3]triazol-6-yl)-6-fluoro-4-(methoxy-d₃)—N-(1-(oxetan-3-yl)piperidin-4-yl)pyrrolo[2,1-f][1,2,4]triazin-2-amine [868];
5-(1-(2,2-difluoroethyl)-1H-benzo[d][1,2,3]triazol-6-yl)-6-fluoro-N-((3S,4R)-3-fluoro-1-methylpiperidin-4-yl)-4-(methoxy-d₃)pyrrolo[2,1-f][1,2,4]triazin-2-amine [869];
5-(1-(2,2-difluoroethyl)-1H-benzo[d][1,2,3]triazol-6-yl)-6-fluoro-N-((3R,4S)-3-fluoro-1-methylpiperidin-4-yl)-4-(methoxy-d₃)pyrrolo[2,1-f][1,2,4]triazin-2-amine [870];
1-((3R,4S)-4-((5-(1-(2,2-difluoroethyl)-1H-benzo[d][1,2,3]triazol-6-yl)-6-fluoro-4-(methoxy-d₃)pyrrolo[2,1-f][1,2,4]triazin-2-yl)amino)-3-fluoropiperidin-1-yl)ethan-1-one [871];
1-((3R,4S)-4-((5-(1-(2,2-difluoroethyl)-1H-benzo[d][1,2,3]triazol-6-yl)-6-fluoro-4-(methoxy-d₃)pyrrolo[2,1-f][1,2,4]triazin-2-yl)amino)-3-fluoropiperidin-1-yl)ethan-1-one-2,2,2-d₃ [872];
1-((3R,4S)-4-((5-(1-(2,2-difluoroethyl)-1H-benzo[d][1,2,3]triazol-6-yl)-6-fluoro-4-(methoxy-d₃)pyrrolo[2,1-f][1,2,4]triazin-2-yl)amino)-3-fluoropiperidin-1-yl)-2-hydroxyethan-1-one [873];
5-(1-(2,2-difluoroethyl)-1H-benzo[d][1,2,3]triazol-6-yl)-6-fluoro-N-((3S,4R)-3-fluoro-1-(oxetan-3-yl)piperidin-4-yl)-4-(methoxy-d₃)pyrrolo[2,1-f][1,2,4]triazin-2-amine [874];
5-(1-(2,2-difluoroethyl)-1H-benzo[d][1,2,3]triazol-6-yl)-6-fluoro-N-((3R,4R)-3-fluoro-1-(oxetan-3-yl)piperidin-4-yl)-4-(methoxy-d₃)pyrrolo[2,1-f][1,2,4]triazin-2-amine [875];
5-(1-(2,2-difluoroethyl)-1H-benzo[d][1,2,3]triazol-6-yl)-6-fluoro-N-((3S,4S)-3-fluoro-1-(oxetan-3-yl)piperidin-4-yl)-4-(methoxy-d₃)pyrrolo[2,1-f][1,2,4]triazin-2-amine [876];
5-(1-(2,2-difluoroethyl)-1H-benzo[d][1,2,3]triazol-6-yl)-6-fluoro-N-((3R,4S)-3-fluoro-1-(oxetan-3-yl)piperidin-4-yl)-4-(methoxy-d₃)pyrrolo[2,1-f][1,2,4]triazin-2-amine [877];
(R)—N-(3,3-difluoro-1-methylpiperidin-4-yl)-5-(1-(2,2-difluoroethyl)-1H-benzo[d][1,2,3]triazol-6-yl)-6-fluoro-4-(methoxy-d₃)pyrrolo[2,1-f][1,2,4]triazin-2-amine [878];
(R)—N-(3,3-difluoro-1-(methyl-d₃)piperidin-4-yl)-5-(1-(2,2-difluoroethyl)-1H-benzo[d][1,2,3]triazol-6-yl)-6-fluoro-4-(methoxy-d₃)pyrrolo[2,1-f][1,2,4]triazin-2-amine [879];
(R)-1-(4-((5-(1-(2,2-difluoroethyl)-1H-benzo[d][1,2,3]triazol-6-yl)-6-fluoro-4-(methoxy-d₃)pyrrolo[2,1-f][1,2,4]triazin-2-yl)amino)-3,3-difluoropiperidin-1-yl)ethan-1-one [880];
(R)-1-(4-((5-(1-(2,2-difluoroethyl)-1H-benzo[d][1,2,3]triazol-6-yl)-6-fluoro-4-(methoxy-d₃)pyrrolo[2,1-f][1,2,4]triazin-2-yl)amino)-3,3-difluoropiperidin-1-yl)ethan-1-one-2,2,2-d₃ [881];
(R)-1-(4-((5-(1-(2,2-difluoroethyl)-1H-benzo[d][1,2,3]triazol-6-yl)-6-fluoro-4-(methoxy-d₃)pyrrolo[2,1-f][1,2,4]triazin-2-yl)amino)-3,3-difluoropiperidin-1-yl)-2-hydroxyethan-1-one [882];
(R)—N-(3,3-difluoro-1-(methylsulfonyl)piperidin-4-yl)-5-(1-(2,2-difluoroethyl)-1H-benzo[d][1,2,3]triazol-6-yl)-6-fluoro-4-(methoxy-d₃)pyrrolo[2,1-f][1,2,4]triazin-2-amine [883];
(R)—N-(3,3-difluoro-1-(oxetan-3-yl)piperidin-4-yl)-5-(1-(2,2-difluoroethyl)-1H-benzo[d][1,2,3]triazol-6-yl)-6-fluoro-4-(methoxy-d₃)pyrrolo[2,1-f][1,2,4]triazin-2-amine [884];
5-(1-(2,2-difluoroethyl)-1H-benzo[d][1,2,3]triazol-6-yl)-6-fluoro-4-(methoxy-d₃)—N-(2-oxaspiro[3.5]nonan-7-yl)pyrrolo[2,1-f][1,2,4]triazin-2-amine 885];
N-((1s,4s)-4-((5-(1-(2,2-difluoroethyl)-4-fluoro-1H-benzo[d][1,2,3]triazol-6-yl)-4-methoxypyrrolo[2,1-f][1,2,4]triazin-2-yl)amino)cyclohexyl)acetamide [886];
2-((3R,4S)-4-((5-(1-(2,2-difluoroethyl)-4-fluoro-1H-benzo[d][1,2,3]triazol-6-yl)-4-methoxypyrrolo[2,1-f][1,2,4]triazin-2-yl)amino)-3-fluoropiperidin-1-yl)ethan-1-ol [887];
1-((3R,4S)-4-((5-(1-(2,2-difluoroethyl)-4-fluoro-1H-benzo[d][1,2,3]triazol-6-yl)-4-methoxypyrrolo[2,1-f][1,2,4]triazin-2-yl)amino)-3-fluoropiperidin-1-yl)-2-hydroxyethan-1-one [888];
5-(1-(2,2-difluoroethyl)-4-fluoro-1H-benzo[d][1,2,3]triazol-6-yl)-N-((3S,4R)-3-fluoro-1-(oxetan-3-yl)piperidin-4-yl)-4-methoxypyrrolo[2,1-f][1,2,4]triazin-2-amine [889];
5-(1-(2,2-difluoroethyl)-4-fluoro-1H-benzo[d][1,2,3]triazol-6-yl)-N-((3R,4R)-3-fluoro-1-(oxetan-3-yl)piperidin-4-yl)-4-methoxypyrrolo[2,1-f][1,2,4]triazin-2-amine [890];
5-(1-(2,2-difluoroethyl)-4-fluoro-1H-benzo[d][1,2,3]triazol-6-yl)-N-((3S,4S)-3-fluoro-1-(oxetan-3-yl)piperidin-4-yl)-4-methoxypyrrolo[2,1-f][1,2,4]triazin-2-amine [891];
(R)-2-(4-((5-(1-(2,2-difluoroethyl)-4-fluoro-1H-benzo[d][1,2,3]triazol-6-yl)-4-methoxypyrrolo[2,1-f][1,2,4]triazin-2-yl)amino)-3,3-difluoropiperidin-1-yl)ethan-1-ol [892];
(R)-1-(4-((5-(1-(2,2-difluoroethyl)-4-fluoro-1H-benzo[d][1,2,3]triazol-6-yl)-4-methoxypyrrolo[2,1-f][1,2,4]triazin-2-yl)amino)-3,3-difluoropiperidin-1-yl)-2-hydroxyethan-1-one [893];
3-((4-methoxy-5-(1-(2,2,2-trifluoroethyl)-1H-benzo[d][1,2,3]triazol-6-yl)pyrrolo[2,1-f][1,2,4]triazin-2-yl)amino)-2,2-dimethylpropanenitrile [894];
N-((1s,4s)-4-((4-methoxy-5-(1-(2,2,2-trifluoroethyl)-1H-benzo[d][1,2,3]triazol-6-yl)pyrrolo[2,1-f][1,2,4]triazin-2-yl)amino)cyclohexyl)acetamide [895];
(R)—N-(4,4-difluoro-1-methylpyrrolidin-3-yl)-4-methoxy-5-(1-(2,2,2-trifluoroethyl)-1H-benzo[d][1,2,3]triazol-6-yl)pyrrolo[2,1-f][1,2,4]triazin-2-amine [896];
(R)-1-(3,3-difluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)pyrrolidin-1-yl)ethan-1-one [897];
(R)—N-(4,4-difluoro-1-(oxetan-3-yl)pyrrolidin-3-yl)-4-methoxy-5-(1-(2,2,2-trifluoroethyl)-1H-benzo[d][1,2,3]triazol-6-yl)pyrrolo[2,1-f][1,2,4]triazin-2-amine [898];
2-((3R,4S)-3-fluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-ol [899]; and
N-((3S,4R)-3-fluoro-1-(2-methoxyethyl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [900]; or a pharmaceutically acceptable salt thereof.

26. The compound of claim 1, wherein the compound of Formula I is selected from the group consisting of
N-((3R,4R)-3-fluoro-1-(2-methoxyethyl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [901];
N-((3S,4S)-3-fluoro-1-(2-methoxyethyl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [902];
N-((3R,4S)-3-fluoro-1-(2-methoxyethyl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [903];
2-((3R,4S)-3-fluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)-N,N-dimethylacetamide [904];
1-((3R,4S)-3-fluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-hydroxyethan-1-one [905];
1-((3R,4S)-3-fluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-(pyrrolidin-1-yl)ethan-1-one [906];
((3R,4S)-3-fluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)(pyrrolidin-1-yl)methanone [907];
N-((3S,4R)-3-fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [908];
N-((3R,4R)-3-fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [909];
N-((3S,4S)-3-fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [910];
N-((3S,4R)-3-fluoro-1-(oxetan-3-yl-3-d)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [911];
N-((3R,4R)-3-fluoro-1-(oxetan-3-yl-3-d)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [912];
N-((3S,4S)-3-fluoro-1-(oxetan-3-yl-3-d)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [913];
N-((3R,4S)-3-fluoro-1-(oxetan-3-yl-3-d)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [914];
(R)-2-(3,3-difluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-ol [915];
(R)—N-(3,3-difluoro-1-(2-methoxyethyl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [916];
(R)-1-(3,3-difluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-hydroxyethan-1-one [917];
(R)-1-(3,3-difluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-(dimethylamino)ethan-1-one [918];
(R)-1-(3,3-difluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-(pyrrolidin-1-yl)ethan-1-one [919];
(R)-(3,3-difluoro-4-((4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)(pyrrolidin-1-yl)methanone [920];
(R)—N-(3,3-difluoro-1-(oxetan-3-yl-3-d)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [921];
(1s,4s)-4-((4-methoxy-5-(1-(2,2,2-trifluoroethyl)-1H-benzo[d][1,2,3]triazol-6-yl)pyrrolo[2,1-f][1,2,4]triazin-2-yl-7-d)amino)-1-methylcyclohexan-1-ol [922];
(1r,4r)-4-((4-methoxy-5-(1-(2,2,2-trifluoroethyl)-1H-benzo[d][1,2,3]triazol-6-yl)pyrrolo[2,1-f][1,2,4]triazin-2-yl-7-d)amino)-1-methylcyclohexan-1-ol [923];
(R)—N-(4,4-difluoro-1-methylpyrrolidin-3-yl)-4-methoxy-5-(1-(2,2,2-trifluoroethyl)-1H-benzo[d][1,2,3]triazol-6-yl)pyrrolo[2,1-f][1,2,4]triazin-7-d-2-amine [924];
(R)-1-(3,3-difluoro-4-((4-methoxy-5-(1-(2,2,2-trifluoroethyl)-1H-benzo[d][1,2,3]triazol-6-yl)pyrrolo[2,1-f][1,2,4]triazin-2-yl-7-d)amino)pyrrolidin-1-yl)ethan-1-one [925];
(R)—N-(4,4-difluoro-1-(oxetan-3-yl)pyrrolidin-3-yl)-4-methoxy-5-(1-(2,2,2-trifluoroethyl)-1H-benzo[d][1,2,3]triazol-6-yl)pyrrolo[2,1-f][1,2,4]triazin-7-d-2-amine [926];
4-methoxy-N-(1-(oxetan-3-yl)piperidin-4-yl)-5-(1-(2,2,2-trifluoroethyl)-1H-benzo[d][1,2,3]triazol-6-yl)pyrrolo[2,1-f][1,2,4]triazin-7-d-2-amine [927];
N-((3S,4R)-3-fluoro-1-methylpiperidin-4-yl)-4-methoxy-5-(1-(2,2,2-trifluoroethyl)-1H-benzo[d][1,2,3]triazol-6-yl)pyrrolo[2,1-f][1,2,4]triazin-7-d-2-amine [928];
N-((3R,4S)-3-fluoro-1-methylpiperidin-4-yl)-4-methoxy-5-(1-(2,2,2-trifluoroethyl)-1H-benzo[d][1,2,3]triazol-6-yl)pyrrolo[2,1-f][1,2,4]triazin-7-d-2-amine [929];
1-((3R,4S)-3-fluoro-4-((4-methoxy-5-(1-(2,2,2-trifluoroethyl)-1H-benzo[d][1,2,3]triazol-6-yl)pyrrolo[2,1-f][1,2,4]triazin-2-yl-7-d)amino)piperidin-1-yl)ethan-1-one [930];
1-((3R,4S)-3-fluoro-4-((4-methoxy-5-(1-(2,2,2-trifluoroethyl)-1H-benzo[d][1,2,3]triazol-6-yl)pyrrolo[2,1-f][1,2,4]triazin-2-yl-7-d)amino)piperidin-1-yl)ethan-1-one-2,2,2-d₃ [931];
1-((3R,4S)-3-fluoro-4-((4-methoxy-5-(1-(2,2,2-trifluoroethyl)-1H-benzo[d][1,2,3]triazol-6-yl)pyrrolo[2,1-f][1,2,4]triazin-2-yl-7-d)amino)piperidin-1-yl)-2-hydroxyethan-1-one [932];
N-((3S,4R)-3-fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-7-d-2-amine [933];
N-((3R,4R)-3-fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-7-d-2-amine [934];
N-((3S,4S)-3-fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-7-d-2-amine [935];
N-((3R,4S)-3-fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-7-d-2-amine [936];
(R)—N-(3,3-difluoro-1-methylpiperidin-4-yl)-4-methoxy-5-(1-(2,2,2-trifluoroethyl)-1H-benzo[d][1,2,3]triazol-6-yl)pyrrolo[2,1-f][1,2,4]triazin-7-d-2-amine [937];
(R)—N-(3,3-difluoro-1-(methyl-d₃)piperidin-4-yl)-4-methoxy-5-(1-(2,2,2-trifluoroethyl)-1H-benzo[d][1,2,3]triazol-6-yl)pyrrolo[2,1-f][1,2,4]triazin-7-d-2-amine [938];
(R)-1-(3,3-difluoro-4-((4-methoxy-5-(1-(2,2,2-trifluoroethyl)-1H-benzo[d][1,2,3]triazol-6-yl)pyrrolo[2,1-f][1,2,4]triazin-2-yl-7-d)amino)piperidin-1-yl)ethan-1-one [939];
(R)-1-(3,3-difluoro-4-((4-methoxy-5-(1-(2,2,2-trifluoroethyl)-1H-benzo[d][1,2,3]triazol-6-yl)pyrrolo[2,1-f][1,2,4]triazin-2-yl-7-d)amino)piperidin-1-yl)ethan-1-one-2,2,2-d₃ [940];
(R)-1-(3,3-difluoro-4-((4-methoxy-5-(1-(2,2,2-trifluoroethyl)-1H-benzo[d][1,2,3]triazol-6-yl)pyrrolo[2,1-f][1,2,4]triazin-2-yl-7-d)amino)piperidin-1-yl)-2-hydroxyethan-1-one [941];
(R)—N-(3,3-difluoro-1-(methylsulfonyl)piperidin-4-yl)-4-methoxy-5-(1-(2,2,2-trifluoroethyl)-1H-benzo[d][1,2,3]triazol-6-yl)pyrrolo[2,1-f][1,2,4]triazin-7-d-2-amine [942];
(R)—N-(3,3-difluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-7-d-2-amine [943];
4-methoxy-N-(2-oxaspiro[3.5]nonan-7-yl)-5-(1-(2,2,2-trifluoroethyl)-1H-benzo[d][1,2,3]triazol-6-yl)pyrrolo[2,1-f][1,2,4]triazin-7-d-2-amine [944];
(1s,4s)-4-((4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)-1-methylcyclohexan-1-ol [945];
(1r,4r)-4-((4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)-1-methylcyclohexan-1-ol [946];
(R)—N-(4,4-difluoro-1-methylpyrrolidin-3-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [947];
(R)-1-(3,3-difluoro-4-((4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)pyrrolidin-1-yl)ethan-1-one [948];
(R)—N-(4,4-difluoro-1-(oxetan-3-yl)pyrrolidin-3-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [949];
4-(methoxy-d₃)—N-(1-(oxetan-3-yl)piperidin-4-yl)-5-(1-(2,2,2-trifluoroethyl)-1H-benzo[d][1,2,3]triazol-6-yl)pyrrolo[2,1-f][1,2,4]triazin-2-amine [950];
N-((3S,4R)-3-fluoro-1-methylpiperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [951];
N-((3R,4S)-3-fluoro-1-methylpiperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [952];
1-((3R,4S)-3-fluoro-4-((4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one [953];
1-((3R,4S)-3-fluoro-4-((4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one-2,2,2-d₃ [954];
1-((3R,4S)-3-fluoro-4-((4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-hydroxyethan-1-one [955];
N-((3S,4R)-3-fluoro-1-(oxetan-3-yl)piperidin-4-yl)-4-(methoxy-d)-5-(1-(2,2,2-trifluoroethyl)-1H-benzo[d][1,2,3]triazol-6-yl)pyrrolo[2,1-f][1,2,4]triazin-2-amine [956];
N-((3R,4R)-3-fluoro-1-(oxetan-3-yl)piperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [957];
N-((3S,4S)-3-fluoro-1-(oxetan-3-yl)piperidin-4-yl)-4-(methoxy-d)-5-(1-(2,2,2-trifluoroethyl)-1H-benzo[d][1,2,3]triazol-6-yl)pyrrolo[2,1-f][1,2,4]triazin-2-amine [958];
N-((3R,4S)-3-fluoro-1-(oxetan-3-yl)piperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [959];
(R)—N-(3,3-difluoro-1-methylpiperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [960];
(R)—N-(3,3-difluoro-1-(methyl-d₃)piperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [961];
(R)-1-(3,3-difluoro-4-((4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one [962];
(R)-1-(3,3-difluoro-4-((4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one-2,2,2-d₃ [963];
(R)-1-(3,3-difluoro-4-((4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-hydroxyethan-1-one [964];
(R)—N-(3,3-difluoro-1-(methylsulfonyl)piperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [965];
(R)—N-(3,3-difluoro-1-(oxetan-3-yl)piperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [966];
4-(methoxy-d₃)—N-(2-oxaspiro[3.5]nonan-7-yl)-5-(1-(2,2,2-trifluoroethyl)-1H-benzo[d][1,2,3]triazol-6-yl)pyrrolo[2,1-f][1,2,4]triazin-2-amine [967];
3-((6-fluoro-4-methoxy-5-(1-(2,2,2-trifluoroethyl)-1H-benzo[d][1,2,3]triazol-6-yl)pyrrolo[2,1-f][1,2,4]triazin-2-yl)amino)-2,2-dimethylpropanenitrile [968];
1-((3S,4R)-3-fluoro-4-((6-fluoro-4-methoxy-5-(1-(2,2,2-trifluoroethyl)-1H-benzo[d][1,2,3]triazol-6-yl)pyrrolo[2,1-f][1,2,4]triazin-2-yl)amino)pyrrolidin-1-yl)ethan-1-one [969];
6-fluoro-N-((3R,4S)-4-fluoro-1-(oxetan-3-yl)pyrrolidin-3-yl)-4-methoxy-5-(1-(2,2,2-trifluoroethyl)-1H-benzo[d][1,2,3]triazol-6-yl)pyrrolo[2,1-f][1,2,4]triazin-2-amine [970];
(R)—N-(4,4-difluoro-1-methylpyrrolidin-3-yl)-6-fluoro-4-methoxy-5-(1-(2,2,2-trifluoroethyl)-1H-benzo[d][1,2,3]triazol-6-yl)pyrrolo[2,1-f][1,2,4]triazin-2-amine [971];
(R)-1-(3,3-difluoro-4-((6-fluoro-4-methoxy-5-(1-(2,2,2-trifluoroethyl)-1H-benzo[d][1,2,3]triazol-6-yl)pyrrolo[2,1-f][1,2,4]triazin-2-yl)amino)pyrrolidin-1-yl)ethan-1-one [972];
(R)—N-(4,4-difluoro-1-(oxetan-3-yl)pyrrolidin-3-yl)-6-fluoro-4-methoxy-5-(1-(2,2,2-trifluoroethyl)-1H-benzo[d][1,2,3]triazol-6-yl)pyrrolo[2,1-f][1,2,4]triazin-2-amine [973];
6-fluoro-4-methoxy-N-(1-(oxetan-3-yl)piperidin-4-yl)-5-(1-(2,2,2-trifluoroethyl)-1H-benzo[d][1,2,3]triazol-6-yl)pyrrolo[2,1-f][1,2,4]triazin-2-amine [974];
6-fluoro-N-((3S,4R)-3-fluoro-1-methylpiperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [975];
6-fluoro-N-((3R,4S)-3-fluoro-1-methylpiperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [976];
6-fluoro-N-((3S,4R)-3-fluoro-1-(2-methoxyethyl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [977];
6-fluoro-N-((3R,4R)-3-fluoro-1-(2-methoxyethyl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [978];
6-fluoro-N-((3S,4S)-3-fluoro-1-(2-methoxyethyl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [979];
6-fluoro-N-((3R,4S)-3-fluoro-1-(2-methoxyethyl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [980];
1-((3R,4S)-3-fluoro-4-((6-fluoro-4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one [981];
1-((3R,4S)-3-fluoro-4-((6-fluoro-4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one-2,2,2-d₃ [982];
1-((3R,4S)-3-fluoro-4-((6-fluoro-4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-hydroxyethan-1-one [983];
6-fluoro-N-((3S,4R)-3-fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [984];
6-fluoro-N-((3R,4R)-3-fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [985];
6-fluoro-N-((3S,4S)-3-fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [986];
6-fluoro-N-((3R,4S)-3-fluoro-1-(oxetan-3-yl)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [987];
6-fluoro-N-((3S,4R)-3-fluoro-1-(oxetan-3-yl-3-d)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [988];
6-fluoro-N-((3R,4R)-3-fluoro-1-(oxetan-3-yl-3-d)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [989];
6-fluoro-N-((3S,4S)-3-fluoro-1-(oxetan-3-yl-3-d)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [990];
6-fluoro-N-((3R,4S)-3-fluoro-1-(oxetan-3-yl-3-d)piperidin-4-yl)-4-methoxy-5-(1-(2,2,2-trifluoroethyl)-1H-benzo[d][1,2,3]triazol-6-yl)pyrrolo[2,1-f][1,2,4]triazin-2-amine [991];
(R)—N-(3,3-difluoro-1-methylpiperidin-4-yl)-6-fluoro-4-methoxy-5-(1-(2,2,2-trifluoroethyl)-1H-benzo[d][1,2,3]triazol-6-yl)pyrrolo[2,1-f][1,2,4]triazin-2-amine [992];
(R)—N-(3,3-difluoro-1-(methyl-d₃)piperidin-4-yl)-6-fluoro-4-methoxy-5-(1-(2,2,2-trifluoroethyl)-1H-benzo[d][1,2,3]triazol-6-yl)pyrrolo[2,1-f][1,2,4]triazin-2-amine [993];
(R)—N-(3,3-difluoro-1-(2-methoxyethyl)piperidin-4-yl)-6-fluoro-4-methoxy-5-(1-(2,2,2-trifluoroethyl)-1H-benzo[d][1,2,3]triazol-6-yl)pyrrolo[2,1-f][1,2,4]triazin-2-amine [994];
(R)-1-(3,3-difluoro-4-((6-fluoro-4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one [995];
(R)-1-(3,3-difluoro-4-((6-fluoro-4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one-2,2,2-d₃ [996];
(R)-1-(3,3-difluoro-4-((6-fluoro-4-methoxy-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-hydroxyethan-1-one [997];
(R)—N-(3,3-difluoro-1-(methylsulfonyl)piperidin-4-yl)-6-fluoro-4-methoxy-5-(1-(2,2,2-trifluoroethyl)-1H-benzo[d][1,2,3]triazol-6-yl)pyrrolo[2,1-f][1,2,4]triazin-2-amine [998];
(R)—N-(3,3-difluoro-1-(oxetan-3-yl)piperidin-4-yl)-6-fluoro-4-methoxy-5-(1-(2,2,2-trifluoroethyl)-1H-benzo[d][1,2,3]triazol-6-yl)pyrrolo[2,1-f][1,2,4]triazin-2-amine [999]; and
(R)—N-(3,3-difluoro-1-(oxetan-3-yl-3-d)piperidin-4-yl)-6-fluoro-4-methoxy-5-(1-(2,2,2-trifluoroethyl)-1H-benzo[d][1,2,3]triazol-6-yl)pyrrolo[2,1-f][1,2,4]triazin-2-amine [1000]; or
a pharmaceutically acceptable salt thereof.

27. The compound of claim 1, wherein the compound of Formula I is selected from the group consisting of:
6-fluoro-4-methoxy-N-(2-oxaspiro[3.5]nonan-7-yl)-5-(1-(2,2,2-trifluoroethyl)-1H-benzo[d][1,2,3]triazol-6-yl)pyrrolo[2,1-f][1,2,4]triazin-2-amine [1001];
(1s,4s)-4-((6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)-1-methylcyclohexan-1-ol [1002];
(1r,4r)-4-((6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)-1-methylcyclohexan-1-ol [1003];
(R)—N-(4,4-difluoro-1-methylpyrrolidin-3-yl)-6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-amine [1004];
(R)-1-(3,3-difluoro-4-((6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)pyrrolidin-1-yl)ethan-1-one [1005];
(R)—N-(4,4-difluoro-1-(oxetan-3-yl)pyrrolidin-3-yl)-6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-amine [1006];
6-fluoro-4-(methoxy-d₃)—N-(1-(oxetan-3-yl)piperidin-4-yl)-5-(1-(2,2,2-trifluoroethyl)-1H-benzo[d][1,2,3]triazol-6-yl)pyrrolo[2,1-f][1,2,4]triazin-2-amine [1007];
6-fluoro-N-((3S,4R)-3-fluoro-1-methylpiperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [1008];
6-fluoro-N-((3R,4S)-3-fluoro-1-methylpiperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [1009];
1-((3R,4S)-3-fluoro-4-((6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one [1010];
1-((3R,4S)-3-fluoro-4-((6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one-2,2,2-d₃ [1011];
1-((3R,4S)-3-fluoro-4-((6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-hydroxyethan-1-one [1012];
6-fluoro-N-((3S,4R)-3-fluoro-1-(oxetan-3-yl)piperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [1013];
6-fluoro-N-((3R,4R)-3-fluoro-1-(oxetan-3-yl)piperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [1014];
6-fluoro-N-((3S,4S)-3-fluoro-1-(oxetan-3-yl)piperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [1015];
6-fluoro-N-((3R,4S)-3-fluoro-1-(oxetan-3-yl)piperidin-4-yl)-4-(methoxy-d₃)-5-(1-(2,2,2-trifluoroethyl)-1H-benzo[d][1,2,3]triazol-6-yl)pyrrolo[2,1-f][1,2,4]triazin-2-amine [1016];
(R)—N-(3,3-difluoro-1-methylpiperidin-4-yl)-6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-amine [1017];
(R)—N-(3,3-difluoro-1-(methyl-d₃)piperidin-4-yl)-6-fluoro-4-(methoxy-d)-5-(1-(2,2,2-trifluoroethyl)-1H-benzo[d][1,2,3]triazol-6-yl)pyrrolo[2,1-f][1,2,4]triazin-2-amine [1018];
(R)-1-(3,3-difluoro-4-((6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one [109];
(R)-1-(3,3-difluoro-4-((6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)piperidin-1-yl)ethan-1-one-2,2,2-d₃ [1020];
(R)-1-(3,3-difluoro-4-((6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-yl)amino)piperidin-1-yl)-2-hydroxyethan-1-one [1021];
(R)—N-(3,3-difluoro-1-(methylsulfonyl)piperidin-4-yl)-6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-amine [1022];
(R)—N-(3,3-difluoro-1-(oxetan-3-yl)piperidin-4-yl)-6-fluoro-4-(methoxy-d₃)-5-(1-(2,2,2-trifluoroethyl)-1H-benzo[d][1,2,3]triazol-6-yl)pyrrolo[2,1-f][1,2,4]triazin-2-amine [1023];
6-fluoro-4-(methoxy-d₃)—N-(2-oxaspiro[3.5]nonan-7-yl)-5-(1-(2,2,2-trifluoroethyl)-1H-benzo[d][1,2,3]triazol-6-yl)pyrrolo[2,1-f][1,2,4]triazin-2-amine [1024];
(1r,4r)-4-((4-methoxy-5-(1-propyl-1H-benzo[d][1,2,3]triazol-6-yl)pyrrolo[2,1-f][1,2,4]triazin-2-yl)amino)-1-methylcyclohexan-1-ol [1025];
(R)-1-(3,3-difluoro-4-((4-methoxy-5-(1-propyl-1H-benzo[d][1,2,3]triazol-6-yl)pyrrolo[2,1-f][1,2,4]triazin-2-yl)amino)piperidin-1-yl)ethan-1-one [1026];
N-((3R,4S)-3-fluoro-1-(oxetan-3-yl)piperidin-4-yl)-4-methoxy-5-(1-propyl-1H-benzo[d][1,2,3]triazol-6-yl)pyrrolo[2,1-f][1,2,4]triazin-2-amine [1027];
3-((5-(1-(3-fluoropropyl)-1H-benzo[d][1,2,3]triazol-6-yl)-4-methoxypyrrolo[2,1-f][1,2,4]triazin-2-yl)amino)-2,2-dimethylpropanenitrile [1028];
(1s,4s)-4-((5-(1-(3-fluoropropyl)-1H-benzo[d][1,2,3]triazol-6-yl)-4-methoxypyrrolo[2,1-f][1,2,4]triazin-2-yl)amino)-1-methylcyclohexan-1-ol [1029];
(1r,4r)-4-((5-(1-(3-fluoropropyl)-1H-benzo[d][1,2,3]triazol-6-yl)-4-methoxypyrrolo[2,1-f][1,2,4]triazin-2-yl)amino)-1-methylcyclohexan-1-ol [1030];
(R)—N-(4,4-difluoro-1-methylpyrrolidin-3-yl)-5-(1-(3-fluoropropyl)-1H-benzo[d][1,2,3]triazol-6-yl)-4-methoxypyrrolo[2,1-f][1,2,4]triazin-2-amine [1031];
(R)-1-(3,3-difluoro-4-((5-(1-(3-fluoropropyl)-1H-benzo[d][1,2,3]triazol-6-yl)-4-methoxypyrrolo[2,1-f][1,2,4]triazin-2-yl)amino)pyrrolidin-1-yl)ethan-1-one [1032];
(R)—N-(4,4-difluoro-1-(oxetan-3-yl)pyrrolidin-3-yl)-5-(1-(3-fluoropropyl)-1H-benzo[d][1,2,3]triazol-6-yl)-4-methoxypyrrolo[2,1-f][1,2,4]triazin-2-amine [1033];
5-(1-(3-fluoropropyl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1034];
N-((3S,4R)-3-fluoro-1-methylpiperidin-4-yl)-5-(1-(3-fluoropropyl)-1H-benzo[d][1,2,3]triazol-6-yl)-4-methoxypyrrolo[2,1-f][1,2,4]triazin-2-amine [1035];
N-((3R,4S)-3-fluoro-1-methylpiperidin-4-yl)-5-(1-(3-fluoropropyl)-1H-benzo[d][1,2,3]triazol-6-yl)-4-methoxypyrrolo[2,1-f][1,2,4]triazin-2-amine [1036];
1-((3R,4S)-3-fluoro-4-((5-(1-(3-fluoropropyl)-1H-benzo[d][1,2,3]triazol-6-yl)-4-methoxypyrrolo[2,1-f][1,2,4]triazin-2-yl)amino)piperidin-1-yl)ethan-1-one [1037];
1-((3R,4S)-3-fluoro-4-((5-(1-(3-fluoropropyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1038];
1-((3R,4S)-3-fluoro-4-((5-(1-(3-fluoropropyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1039];
N-((3S,4R)-3-fluoro-1-(oxetan-3-yl)piperidin-4-yl)-5-(1-(3-fluoropropyl)-1H-benzo[d][1,2,3]triazol-6-yl)-4-methoxypyrrolo[2,1-f][1,2,4]triazin-2-amine [1040];
N-((3R,4R)-3-fluoro-1-(oxetan-3-yl)piperidin-4-yl)-5-(1-(3-fluoropropyl)-1H-benzo[d][1,2,3]triazol-6-yl)-4-methoxypyrrolo[2,1-f][1,2,4]triazin-2-amine [1041];
N-((3S,4S)-3-fluoro-1-(oxetan-3-yl)piperidin-4-yl)-5-(1-(3-fluoropropyl)-1H-benzo[d][1,2,3]triazol-6-yl)-4-methoxypyrrolo[2,1-f][1,2,4]triazin-2-amine [1042];
N-((3R,4S)-3-fluoro-1-(oxetan-3-yl)piperidin-4-yl)-5-(1-(3-fluoropropyl)-1H-benzo[d][1,2,3]triazol-6-yl)-4-methoxypyrrolo[2,1-f][1,2,4]triazin-2-amine [1043];
(R)—N-(3,3-difluoro-1-methylpiperidin-4-yl)-5-(1-(3-fluoropropyl)-1H-benzo[d][1,2,3]triazol-6-yl)-4-methoxypyrrolo[2,1-f][1,2,4]triazin-2-amine [1044];
(R)—N-(3,3-difluoro-1-(methyl-d₃)piperidin-4-yl)-5-(1-(3-fluoropropyl)-1H-benzo[d][1,2,3]triazol-6-yl)-4-methoxypyrrolo[2,1-f][1,2,4]triazin-2-amine [1045];
(R)-1-(3,3-difluoro-4-((5-(1-(3-fluoropropyl)-1H-benzo[d][1,2,3]triazol-6-yl)-4-methoxypyrrolo[2,1-f][1,2,4]triazin-2-yl)amino)piperidin-1-yl)ethan-1-one [1046];
(R)-1-(3,3-difluoro-4-((5-(1-(3-fluoropropyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1047];
(R)-1-(3,3-difluoro-4-((5-(1-(3-fluoropropyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1048];
(R)—N-(3,3-difluoro-1-(methylsulfonyl)piperidin-4-yl)-5-(1-(3-fluoropropyl)-1H-benzo[d][1,2,3]triazol-6-yl)-4-methoxypyrrolo[2,1-f][1,2,4]triazin-2-amine [1049];
(R)—N-(3,3-difluoro-1-(oxetan-3-yl)piperidin-4-yl)-5-(1-(3-fluoropropyl)-1H-benzo[d][1,2,3]triazol-6-yl)-4-methoxypyrrolo[2,1-f][1,2,4]triazin-2-amine [1050];
5-(1-(3-fluoropropyl)-1H-benzo[d][1,2,3]triazol-6-yl)-4-methoxy-N-(2-oxaspiro[3.5]nonan-7-yl)pyrrolo[2,1-f][1,2,4]triazin-2-amine [1051];
3-((6-fluoro-5-(1-(3-fluoropropyl)-1H-benzo[d][1,2,3]triazol-6-yl)-4-methoxypyrrolo[2,1-f][1,2,4]triazin-2-yl)amino)-2,2-dimethylpropanenitrile [1052];
(1s,4s)-4-((6-fluoro-5-(1-(3-fluoropropyl)-1H-benzo[d][1,2,3]triazol-6-yl)-4-methoxypyrrolo[2,1-f][1,2,4]triazin-2-yl)amino)-1-methylcyclohexan-1-ol [1053];
(1r,4r)-4-((6-fluoro-5-(1-(3-fluoropropyl)-1H-benzo[d][1,2,3]triazol-6-yl)-4-methoxypyrrolo[2,1-f][1,2,4]triazin-2-yl)amino)-1-methylcyclohexan-1-ol [1054];
(R)—N-(4,4-difluoro-1-methylpyrrolidin-3-yl)-6-fluoro-5-(1-(3-fluoropropyl)-1H-benzo[d][1,2,3]triazol-6-yl)-4-methoxypyrrolo[2,1-f][1,2,4]triazin-2-amine [1055];
(R)-1-(3,3-difluoro-4-((6-fluoro-5-(1-(3-fluoropropyl)-1H-benzo[d][1,2,3]triazol-6-yl)-4-methoxypyrrolo[2,1-f][1,2,4]triazin-2-yl)amino)pyrrolidin-1-yl)ethan-1-one [1056];
(R)—N-(4,4-difluoro-1-(oxetan-3-yl)pyrrolidin-3-yl)-6-fluoro-5-(1-(3-fluoropropyl)-1H-benzo[d][1,2,3]triazol-6-yl)-4-methoxypyrrolo[2,1-f][1,2,4]triazin-2-amine [1057];
6-fluoro-5-(1-(3-fluoropropyl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1058];
6-fluoro-N-((3S,4R)-3-fluoro-1-methylpiperidin-4-yl)-5-(1-(3-fluoropropyl)-1H-benzo[d][1,2,3]triazol-6-yl)-4-methoxypyrrolo[2,1-f][1,2,4]triazin-2-amine [1059];
6-fluoro-N-((3R,4S)-3-fluoro-1-methylpiperidin-4-yl)-5-(1-(3-fluoropropyl)-1H-benzo[d][1,2,3]triazol-6-yl)-4-methoxypyrrolo[2,1-f][1,2,4]triazin-2-amine [1060];
1-((3R,4S)-3-fluoro-4-((6-fluoro-5-(1-(3-fluoropropyl)-1H-benzo[d][1,2,3]triazol-6-yl)-4-methoxypyrrolo[2,1-f][1,2,4]triazin-2-yl)amino)piperidin-1-yl)ethan-1-one [1061];
1-((3R,4S)-3-fluoro-4-((6-fluoro-5-(1-(3-fluoropropyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1062];
1-((3R,4S)-3-fluoro-4-((6-fluoro-5-(1-(3-fluoropropyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1063];
6-fluoro-N-((3S,4R)-3-fluoro-1-(oxetan-3-yl)piperidin-4-yl)-5-(1-(3-fluoropropyl)-1H-benzo[d][1,2,3]triazol-6-yl)-4-methoxypyrrolo[2,1-f][1,2,4]triazin-2-amine [1064];
6-fluoro-N-((3R,4R)-3-fluoro-1-(oxetan-3-yl)piperidin-4-yl)-5-(1-(3-fluoropropyl)-1H-benzo[d][1,2,3]triazol-6-yl)-4-methoxypyrrolo[2,1-f][1,2,4]triazin-2-amine [1065];
6-fluoro-N-((3S,4S)-3-fluoro-1-(oxetan-3-yl)piperidin-4-yl)-5-(1-(3-fluoropropyl)-1H-benzo[d][1,2,3]triazol-6-yl)-4-methoxypyrrolo[2,1-f][1,2,4]triazin-2-amine [1066];
6-fluoro-N-((3R,4S)-3-fluoro-1-(oxetan-3-yl)piperidin-4-yl)-5-(1-(3-fluoropropyl)-1H-benzo[d][1,2,3]triazol-6-yl)-4-methoxypyrrolo[2,1-f][1,2,4]triazin-2-amine [1067];
(R)—N-(3,3-difluoro-1-methylpiperidin-4-yl)-6-fluoro-5-(1-(3-fluoropropyl)-1H-benzo[d][1,2,3]triazol-6-yl)-4-methoxypyrrolo[2,1-f][1,2,4]triazin-2-amine [1068];
(R)—N-(3,3-difluoro-1-(methyl-d₃)piperidin-4-yl)-6-fluoro-5-(1-(3-fluoropropyl)-1H-benzo[d][1,2,3]triazol-6-yl)-4-methoxypyrrolo[2,1-f][1,2,4]triazin-2-amine [1069];
(R)-1-(3,3-difluoro-4-((6-fluoro-5-(1-(3-fluoropropyl)-1H-benzo[d][1,2,3]triazol-6-yl)-4-methoxypyrrolo[2,1-f][1,2,4]triazin-2-yl)amino)piperidin-1-yl)ethan-1-one [1070];
(R)-1-(3,3-difluoro-4-((6-fluoro-5-(1-(3-fluoropropyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1071];
(R)-1-(3,3-difluoro-4-((6-fluoro-5-(1-(3-fluoropropyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1072];
(R)—N-(3,3-difluoro-1-(methylsulfonyl)piperidin-4-yl)-6-fluoro-5-(1-(3-fluoropropyl)-1H-benzo[d][1,2,3]triazol-6-yl)-4-methoxypyrrolo[2,1-f][1,2,4]triazin-2-amine [1073];
(R)—N-(3,3-difluoro-1-(oxetan-3-yl)piperidin-4-yl)-6-fluoro-5-(1-(3-fluoropropyl)-1H-benzo[d][1,2,3]triazol-6-yl)-4-methoxypyrrolo[2,1-f][1,2,4]triazin-2-amine [1074];
6-fluoro-5-(1-(3-fluoropropyl)-1H-benzo[d][1,2,3]triazol-6-yl)-4-methoxy-N-(2-oxaspiro[3.5]nonan-7-yl)pyrrolo[2,1-f][1,2,4]triazin-2-amine [1075];
(S)-3-((5-(1-(2-fluoropropyl)-1H-benzo[d][1,2,3]triazol-6-yl)-4-methoxypyrrolo[2,1-f][1,2,4]triazin-2-yl)amino)-2,2-dimethylpropanenitrile [1076];
(1R,4s)-4-((5-(1-((S)-2-fluoropropyl)-1H-benzo[d][1,2,3]triazol-6-yl)-4-methoxypyrrolo[2,1-f][1,2,4]triazin-2-yl)amino)-1-methylcyclohexan-1-ol [1077];
(1S,4r)-4-((5-(1-((S)-2-fluoropropyl)-1H-benzo[d][1,2,3]triazol-6-yl)-4-methoxypyrrolo[2,1-f][1,2,4]triazin-2-yl)amino)-1-methylcyclohexan-1-ol [1078];
N—((R)-4,4-difluoro-1-methylpyrrolidin-3-yl)-5-(1-((S)-2-fluoropropyl)-1H-benzo[d][1,2,3]triazol-6-yl)-4-methoxypyrrolo[2,1-f][1,2,4]triazin-2-amine [1079];
1-((R)-3,3-difluoro-4-((5-(1-((S)-2-fluoropropyl)-1H-benzo[d][1,2,3]triazol-6-yl)-4-methoxypyrrolo[2,1-f][1,2,4]triazin-2-yl)amino)pyrrolidin-1-yl)ethan-1-one [1080];
N—((R)-4,4-difluoro-1-(oxetan-3-yl)pyrrolidin-3-yl)-5-(1-((S)-2-fluoropropyl)-1H-benzo[d][1,2,3]triazol-6-yl)-4-methoxypyrrolo[2,1-f][1,2,4]triazin-2-amine [1081];
(S)-5-(1-(2-fluoropropyl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1082];
N-((3S,4R)-3-fluoro-1-methylpiperidin-4-yl)-5-(1-((S)-2-fluoropropyl)-1H-benzo[d][1,2,3]triazol-6-yl)-4-methoxypyrrolo[2,1-f][1,2,4]triazin-2-amine [1083];
N-((3R,4S)-3-fluoro-1-methylpiperidin-4-yl)-5-(1-((S)-2-fluoropropyl)-1H-benzo[d][1,2,3]triazol-6-yl)-4-methoxypyrrolo[2,1-f][1,2,4]triazin-2-amine [1084];
1-((3R,4S)-3-fluoro-4-((5-(1-((S)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one [1085];
1-((3R,4S)-3-fluoro-4-((5-(1-((S)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1086];
1-((3R,4S)-3-fluoro-4-((5-(1-((S)-2-fluoropropyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1087];
N-((3S,4R)-3-fluoro-1-(oxetan-3-yl)piperidin-4-yl)-5-(1-((S)-2-fluoropropyl)-1H-benzo[d][1,2,3]triazol-6-yl)-4-methoxypyrrolo[2,1-f][1,2,4]triazin-2-amine [1088];
N-((3R,4R)-3-fluoro-1-(oxetan-3-yl)piperidin-4-yl)-5-(1-((S)-2-fluoropropyl)-1H-benzo[d][1,2,3]triazol-6-yl)-4-methoxypyrrolo[2,1-f][1,2,4]triazin-2-amine [1089];
N-((3S,4S)-3-fluoro-1-(oxetan-3-yl)piperidin-4-yl)-5-(1-((S)-2-fluoropropyl)-1H-benzo[d][1,2,3]triazol-6-yl)-4-methoxypyrrolo[2,1-f][1,2,4]triazin-2-amine [1090];
N-((3R,4S)-3-fluoro-1-(oxetan-3-yl)piperidin-4-yl)-5-(1-((S)-2-fluoropropyl)-1H-benzo[d][1,2,3]triazol-6-yl)-4-methoxypyrrolo[2,1-f][1,2,4]triazin-2-amine [1091];
N—((R)-3,3-difluoro-1-methylpiperidin-4-yl)-5-(1-((S)-2-fluoropropyl)-1H-benzo[d][1,2,3]triazol-6-yl)-4-methoxypyrrolo[2,1-f][1,2,4]triazin-2-amine [1092];
N—((R)-3,3-difluoro-1-(methyl-d₃)piperidin-4-yl)-5-(1-((S)-2-fluoropropyl)-1H-benzo[d][1,2,3]triazol-6-yl)-4-methoxypyrrolo[2,1-f][1,2,4]triazin-2-amine [1093];
1-((R)-3,3-difluoro-4-((5-(1-((S)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one [1094];
1-((R)-3,3-difluoro-4-((5-(1-((S)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1095];
1-((R)-3,3-difluoro-4-((5-(1-((S)-2-fluoropropyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1096];
N—((R)-3,3-difluoro-1-(methylsulfonyl)piperidin-4-yl)-5-(1-((S)-2-fluoropropyl)-1H-benzo[d][1,2,3]triazol-6-yl)-4-methoxypyrrolo[2,1-f][1,2,4]triazin-2-amine [1097];
N—((R)-3,3-difluoro-1-(oxetan-3-yl)piperidin-4-yl)-5-(1-((S)-2-fluoropropyl)-1H-benzo[d][1,2,3]triazol-6-yl)-4-methoxypyrrolo[2,1-f][1,2,4]triazin-2-amine [1098];
(S)-5-(1-(2-fluoropropyl)-1H-benzo[d][1,2,3]triazol-6-yl)-4-methoxy-N-(2-oxaspiro[3.5]nonan-7-yl)pyrrolo[2,1-f][1,2,4]triazin-2-amine [1099]; and
(R)-3-((5-(1-(2-fluoropropyl)-1H-benzo[d][1,2,3]triazol-6-yl)-4-methoxypyrrolo[2,1-f][1,2,4]triazin-2-yl)amino)-2,2-dimethylpropanenitrile [1100]; or a pharmaceutically acceptable salt thereof.

28. The compound of claim 1, wherein the compound of Formula I is selected from the group consisting of
(1S,4s)-4-((5-(1-((R)-2-fluoropropyl)-1H-benzo[d][1,2,3]triazol-6-yl)-4-methoxypyrrolo[2,1-f][1,2,4]triazin-2-yl)amino)-1-methylcyclohexan-1-ol [1101];
(1R,4r)-4-((5-(1-((R)-2-fluoropropyl)-1H-benzo[d][1,2,3]triazol-6-yl)-4-methoxypyrrolo[2,1-f][1,2,4]triazin-2-yl)amino)-1-methylcyclohexan-1-ol [1102];
N—((R)-4,4-difluoro-1-methylpyrrolidin-3-yl)-5-(1-((R)-2-fluoropropyl)-1H-benzo[d][1,2,3]triazol-6-yl)-4-methoxypyrrolo[2,1-f][1,2,4]triazin-2-amine [1103];
1-((R)-3,3-difluoro-4-((5-(1-((R)-2-fluoropropyl)-1H-benzo[d][1,2,3]triazol-6-yl)-4-methoxypyrrolo[2,1-f][1,2,4]triazin-2-yl)amino)pyrrolidin-1-yl)ethan-1-one [1104];
N—((R)-4,4-difluoro-1-(oxetan-3-yl)pyrrolidin-3-yl)-5-(1-((R)-2-fluoropropyl)-1H-benzo[d][1,2,3]triazol-6-yl)-4-methoxypyrrolo[2,1-f][1,2,4]triazin-2-amine [1105];
(R)-5-(1-(2-fluoropropyl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1106];
N-((3S,4R)-3-fluoro-1-methylpiperidin-4-yl)-5-(1-((R)-2-fluoropropyl)-1H-benzo[d][1,2,3]triazol-6-yl)-4-methoxypyrrolo[2,1-f][1,2,4]triazin-2-amine [1107];
N-((3R,4S)-3-fluoro-1-methylpiperidin-4-yl)-5-(1-((R)-2-fluoropropyl)-1H-benzo[d][1,2,3]triazol-6-yl)-4-methoxypyrrolo[2,1-f][1,2,4]triazin-2-amine [1108];
1-((3R,4S)-3-fluoro-4-((5-(1-((R)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one [1109];
1-((3R,4S)-3-fluoro-4-((5-(1-((R)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1110];
1-((3R,4S)-3-fluoro-4-((5-(1-((R)-2-fluoropropyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1111];
N-((3S,4R)-3-fluoro-1-(oxetan-3-yl)piperidin-4-yl)-5-(1-((R)-2-fluoropropyl)-1H-benzo[d][1,2,3]triazol-6-yl)-4-methoxypyrrolo[2,1-f][1,2,4]triazin-2-amine [1112];
N-((3R,4R)-3-fluoro-1-(oxetan-3-yl)piperidin-4-yl)-5-(1-((R)-2-fluoropropyl)-1H-benzo[d][1,2,3]triazol-6-yl)-4-methoxypyrrolo[2,1-f][1,2,4]triazin-2-amine [1113];
N-((3S,4S)-3-fluoro-1-(oxetan-3-yl)piperidin-4-yl)-5-(1-((R)-2-fluoropropyl)-1H-benzo[d][1,2,3]triazol-6-yl)-4-methoxypyrrolo[2,1-f][1,2,4]triazin-2-amine [1114];
N-((3R,4S)-3-fluoro-1-(oxetan-3-yl)piperidin-4-yl)-5-(1-((R)-2-fluoropropyl)-1H-benzo[d][1,2,3]triazol-6-yl)-4-methoxypyrrolo[2,1-f][1,2,4]triazin-2-amine [1115];
N—((R)-3,3-difluoro-1-methylpiperidin-4-yl)-5-(1-((R)-2-fluoropropyl)-1H-benzo[d][1,2,3]triazol-6-yl)-4-methoxypyrrolo[2,1-f][1,2,4]triazin-2-amine [1116];
N—((R)-3,3-difluoro-1-(methyl-d₃)piperidin-4-yl)-5-(1-((R)-2-fluoropropyl)-1H-benzo[d][1,2,3]triazol-6-yl)-4-methoxypyrrolo[2,1-f][1,2,4]triazin-2-amine [1117];
1-((R)-3,3-difluoro-4-((5-(1-((R)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one [1118];
1-((R)-3,3-difluoro-4-((5-(1-((R)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1119];
1-((R)-3,3-difluoro-4-((5-(1-((R)-2-fluoropropyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1120];
N—((R)-3,3-difluoro-1-(methylsulfonyl)piperidin-4-yl)-5-(1-((R)-2-fluoropropyl)-1H-benzo[d][1,2,3]triazol-6-yl)-4-methoxypyrrolo[2,1-f][1,2,4]triazin-2-amine [1121];
N—((R)-3,3-difluoro-1-(oxetan-3-yl)piperidin-4-yl)-5-(1-((R)-2-fluoropropyl)-1H-benzo[d][1,2,3]triazol-6-yl)-4-methoxypyrrolo[2,1-f][1,2,4]triazin-2-amine [1122];
(R)-5-(1-(2-fluoropropyl)-1H-benzo[d][1,2,3]triazol-6-yl)-4-methoxy-N-(2-oxaspiro[3.5]nonan-7-yl)pyrrolo[2,1-f][1,2,4]triazin-2-amine [1123];
(S)-3-((6-fluoro-5-(1-(2-fluoropropyl)-1H-benzo[d][1,2,3]triazol-6-yl)-4-methoxypyrrolo[2,1-f][1,2,4]triazin-2-yl)amino)-2,2-dimethylpropanenitrile [1124];
(1R,4s)-4-((6-fluoro-5-(1-((S)-2-fluoropropyl)-1H-benzo[d][1,2,3]triazol-6-yl)-4-methoxypyrrolo[2,1-f][1,2,4]triazin-2-yl)amino)-1-methylcyclohexan-1-ol [1125];
(1S,4r)-4-((6-fluoro-5-(1-((S)-2-fluoropropyl)-1H-benzo[d][1,2,3]triazol-6-yl)-4-methoxypyrrolo[2,1-f][1,2,4]triazin-2-yl)amino)-1-methylcyclohexan-1-ol [1126];
N—((R)-4,4-difluoro-1-methylpyrrolidin-3-yl)-6-fluoro-5-(1-((S)-2-fluoropropyl)-1H-benzo[d][1,2,3]triazol-6-yl)-4-methoxypyrrolo[2,1-f][1,2,4]triazin-2-amine [1127];
1-((R)-3,3-difluoro-4-((6-fluoro-5-(1-((S)-2-fluoropropyl)-1H-benzo[d][1,2,3]triazol-6-yl)-4-methoxypyrrolo[2,1-f][1,2,4]triazin-2-yl)amino)pyrrolidin-1-yl)ethan-1-one [1128];
N—((R)-4,4-difluoro-1-(oxetan-3-yl)pyrrolidin-3-yl)-6-fluoro-5-(1-((S)-2-fluoropropyl)-1H-benzo[d][1,2,3]triazol-6-yl)-4-methoxypyrrolo[2,1-f][1,2,4]triazin-2-amine [1129];
(S)-6-fluoro-5-(1-(2-fluoropropyl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1130];
6-fluoro-N-((3S,4R)-3-fluoro-1-methylpiperidin-4-yl)-5-(1-((S)-2-fluoropropyl)-1H-benzo[d][1,2,3]triazol-6-yl)-4-methoxypyrrolo[2,1-f][1,2,4]triazin-2-amine [1131];
6-fluoro-N-((3R,4S)-3-fluoro-1-methylpiperidin-4-yl)-5-(1-((S)-2-fluoropropyl)-1H-benzo[d][1,2,3]triazol-6-yl)-4-methoxypyrrolo[2,1-f][1,2,4]triazin-2-amine [1132];
1-((3R,4S)-3-fluoro-4-((6-fluoro-5-(1-((S)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one [1133];
1-((3R,4S)-3-fluoro-4-((6-fluoro-5-(1-((S)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1134];
1-((3R,4S)-3-fluoro-4-((6-fluoro-5-(1-((S)-2-fluoropropyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1135];
6-fluoro-N-((3S,4R)-3-fluoro-1-(oxetan-3-yl)piperidin-4-yl)-5-(1-((S)-2-fluoropropyl)-1H-benzo[d][1,2,3]triazol-6-yl)-4-methoxypyrrolo[2,1-f][1,2,4]triazin-2-amine [1136];
6-fluoro-N-((3R,4R)-3-fluoro-1-(oxetan-3-yl)piperidin-4-yl)-5-(1-((S)-2-fluoropropyl)-1H-benzo[d][1,2,3]triazol-6-yl)-4-methoxypyrrolo[2,1-f][1,2,4]triazin-2-amine [1137];
6-fluoro-N-((3S,4S)-3-fluoro-1-(oxetan-3-yl)piperidin-4-yl)-5-(1-((S)-2-fluoropropyl)-1H-benzo[d][1,2,3]triazol-6-yl)-4-methoxypyrrolo[2,1-f][1,2,4]triazin-2-amine [1138];
6-fluoro-N-((3R,4S)-3-fluoro-1-(oxetan-3-yl)piperidin-4-yl)-5-(1-((S)-2-fluoropropyl)-1H-benzo[d][1,2,3]triazol-6-yl)-4-methoxypyrrolo[2,1-f][1,2,4]triazin-2-amine [1139];
N—((R)-3,3-difluoro-1-methylpiperidin-4-yl)-6-fluoro-5-(1-((S)-2-fluoropropyl)-1H-benzo[d][1,2,3]triazol-6-yl)-4-methoxypyrrolo[2,1-f][1,2,4]triazin-2-amine [1140];
N—((R)-3,3-difluoro-1-(methyl-d₃)piperidin-4-yl)-6-fluoro-5-(1-((S)-2-fluoropropyl)-1H-benzo[d][1,2,3]triazol-6-yl)-4-methoxypyrrolo[2,1-f][1,2,4]triazin-2-amine [1141];
1-((R)-3,3-difluoro-4-((6-fluoro-5-(1-((S)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one [1142];
1-((R)-3,3-difluoro-4-((6-fluoro-5-(1-((S)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1143];
1-((R)-3,3-difluoro-4-((6-fluoro-5-(1-((S)-2-fluoropropyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1144];
N—((R)-3,3-difluoro-1-(methylsulfonyl)piperidin-4-yl)-6-fluoro-5-(1-((S)-2-fluoropropyl)-1H-benzo[d][1,2,3]triazol-6-yl)-4-methoxypyrrolo[2,1-f][1,2,4]triazin-2-amine [1145];
N—((R)-3,3-difluoro-1-(oxetan-3-yl)piperidin-4-yl)-6-fluoro-5-(1-((S)-2-fluoropropyl)-1H-benzo[d][1,2,3]triazol-6-yl)-4-methoxypyrrolo[2,1-f][1,2,4]triazin-2-amine [1146];
(S)-6-fluoro-5-(1-(2-fluoropropyl)-1H-benzo[d][1,2,3]triazol-6-yl)-4-methoxy-N-(2-oxaspiro[3.5]nonan-7-yl)pyrrolo[2,1-f][1,2,4]triazin-2-amine [1147];
(R)-3-((6-fluoro-5-(1-(2-fluoropropyl)-1H-benzo[d][1,2,3]triazol-6-yl)-4-methoxypyrrolo[2,1-f][1,2,4]triazin-2-yl)amino)-2,2-dimethylpropanenitrile [1148];
(1S,4s)-4-((6-fluoro-5-(1-((R)-2-fluoropropyl)-1H-benzo[d][1,2,3]triazol-6-yl)-4-methoxypyrrolo[2,1-f][1,2,4]triazin-2-yl)amino)-1-methylcyclohexan-1-ol [1149];
(1R,4r)-4-((6-fluoro-5-(1-((R)-2-fluoropropyl)-1H-benzo[d][1,2,3]triazol-6-yl)-4-methoxypyrrolo[2,1-f][1,2,4]triazin-2-yl)amino)-1-methylcyclohexan-1-ol [1150];
N—((R)-4,4-difluoro-1-methylpyrrolidin-3-yl)-6-fluoro-5-(1-((R)-2-fluoropropyl)-1H-benzo[d][1,2,3]triazol-6-yl)-4-methoxypyrrolo[2,1-f][1,2,4]triazin-2-amine [1151];
1-((R)-3,3-difluoro-4-((6-fluoro-5-(1-((R)-2-fluoropropyl)-1H-benzo[d][1,2,3]triazol-6-yl)-4-methoxypyrrolo[2,1-f][1,2,4]triazin-2-yl)amino)pyrrolidin-1-yl)ethan-1-one [1152];
N—((R)-4,4-difluoro-1-(oxetan-3-yl)pyrrolidin-3-yl)-6-fluoro-5-(1-((R)-2-fluoropropyl)-1H-benzo[d][1,2,3]triazol-6-yl)-4-methoxypyrrolo[2,1-f][1,2,4]triazin-2-amine [1153];
(R)-6-fluoro-5-(1-(2-fluoropropyl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1154];
6-fluoro-N-((3S,4R)-3-fluoro-1-methylpiperidin-4-yl)-5-(1-((R)-2-fluoropropyl)-1H-benzo[d][1,2,3]triazol-6-yl)-4-methoxypyrrolo[2,1-f][1,2,4]triazin-2-amine [1155];
6-fluoro-N-((3R,4S)-3-fluoro-1-methylpiperidin-4-yl)-5-(1-((R)-2-fluoropropyl)-1H-benzo[d][1,2,3]triazol-6-yl)-4-methoxypyrrolo[2,1-f][1,2,4]triazin-2-amine [1156];
1-((3R,4S)-3-fluoro-4-((6-fluoro-5-(1-((R)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one [1157];
1-((3R,4S)-3-fluoro-4-((6-fluoro-5-(1-((R)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1158];
1-((3R,4S)-3-fluoro-4-((6-fluoro-5-(1-((R)-2-fluoropropyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1159];
6-fluoro-N-((3S,4R)-3-fluoro-1-(oxetan-3-yl)piperidin-4-yl)-5-(1-((R)-2-fluoropropyl)-1H-benzo[d][1,2,3]triazol-6-yl)-4-methoxypyrrolo[2,1-f][1,2,4]triazin-2-amine [1160];
6-fluoro-N-((3R,4R)-3-fluoro-1-(oxetan-3-yl)piperidin-4-yl)-5-(1-((R)-2-fluoropropyl)-1H-benzo[d][1,2,3]triazol-6-yl)-4-methoxypyrrolo[2,1-f][1,2,4]triazin-2-amine [1161];
6-fluoro-N-((3S,4S)-3-fluoro-1-(oxetan-3-yl)piperidin-4-yl)-5-(1-((R)-2-fluoropropyl)-1H-benzo[d][1,2,3]triazol-6-yl)-4-methoxypyrrolo[2,1-f][1,2,4]triazin-2-amine [1162];
6-fluoro-N-((3R,4S)-3-fluoro-1-(oxetan-3-yl)piperidin-4-yl)-5-(1-((R)-2-fluoropropyl)-1H-benzo[d][1,2,3]triazol-6-yl)-4-methoxypyrrolo[2,1-f][1,2,4]triazin-2-amine [1163];
N—((R)-3,3-difluoro-1-methylpiperidin-4-yl)-6-fluoro-5-(1-((R)-2-fluoropropyl)-1H-benzo[d][1,2,3]triazol-6-yl)-4-methoxypyrrolo[2,1-f][1,2,4]triazin-2-amine [1164];
N—((R)-3,3-difluoro-1-(methyl-d₃)piperidin-4-yl)-6-fluoro-5-(1-((R)-2-fluoropropyl)-1H-benzo[d][1,2,3]triazol-6-yl)-4-methoxypyrrolo[2,1-f][1,2,4]triazin-2-amine [1165];
1-((R)-3,3-difluoro-4-((6-fluoro-5-(1-((R)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one [1166];
1-((R)-3,3-difluoro-4-((6-fluoro-5-(1-((R)-2-fluoropropyl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1167];
1-((R)-3,3-difluoro-4-((6-fluoro-5-(1-((R)-2-fluoropropyl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1168];
N—((R)-3,3-difluoro-1-(methylsulfonyl)piperidin-4-yl)-6-fluoro-5-(1-((R)-2-fluoropropyl)-1H-benzo[d][1,2,3]triazol-6-yl)-4-methoxypyrrolo[2,1-f][1,2,4]triazin-2-amine [1169];
N—((R)-3,3-difluoro-1-(oxetan-3-yl)piperidin-4-yl)-6-fluoro-5-(1-((R)-2-fluoropropyl)-1H-benzo[d][1,2,3]triazol-6-yl)-4-methoxypyrrolo[2,1-f][1,2,4]triazin-2-amine [1170];
(R)-6-fluoro-5-(1-(2-fluoropropyl)-1H-benzo[d][1,2,3]triazol-6-yl)-4-methoxy-N-(2-oxaspiro[3.5]nonan-7-yl)pyrrolo[2,1-f][1,2,4]triazin-2-amine [1171];
(S)-3-((5-(1-(1-fluoropropan-2-yl)-1H-benzo[d][1,2,3]triazol-6-yl)-4-methoxypyrrolo[2,1-f][1,2,4]triazin-2-yl)amino)-2,2-dimethylpropanenitrile [1172];
N-((1R,4s)-4-((5-(1-((S)-1-fluoropropan-2-yl)-1H-benzo[d][1,2,3]triazol-6-yl)-4-methoxypyrrolo[2,1-f][1,2,4]triazin-2-yl)amino)cyclohexyl)acetamide [1173];
(1R,4s)-4-((5-(1-((S)-1-fluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1174];
(1S,4r)-4-((5-(1-((S)-1-fluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1175];
N—((R)-4,4-difluoro-1-methylpyrrolidin-3-yl)-5-(1-((S)-1-fluoropropan-2-yl)-1H-benzo[d][1,2,3]triazol-6-yl)-4-methoxypyrrolo[2,1-f][1,2,4]triazin-2-amine [1176];
1-((R)-3,3-difluoro-4-((5-(1-((S)-1-fluoropropan-2-yl)-1H-benzo[d][1,2,3]triazol-6-yl)-4-methoxypyrrolo[2,1-f][1,2,4]triazin-2-yl)amino)pyrrolidin-1-yl)ethan-1-one [1177];
N—((R)-4,4-difluoro-1-(oxetan-3-yl)pyrrolidin-3-yl)-5-(1-((S)-1-fluoropropan-2-yl)-1H-benzo[d][1,2,3]triazol-6-yl)-4-methoxypyrrolo[2,1-f][1,2,4]triazin-2-amine [1178];
(S)-5-(1-(1-fluoropropan-2-yl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1179];
N-((3S,4R)-3-fluoro-1-methylpiperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1180];
N-((3R,4S)-3-fluoro-1-methylpiperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1181];
1-((3R,4S)-3-fluoro-4-((5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one [1182];
1-((3R,4S)-3-fluoro-4-((5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1183];
1-((3R,4S)-3-fluoro-4-((5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1184];
N-((3S,4R)-3-fluoro-1-(oxetan-3-yl)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1185];
N-((3R,4R)-3-fluoro-1-(oxetan-3-yl)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1186];
N-((3S,4S)-3-fluoro-1-(oxetan-3-yl)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1187];
N-((3R,4S)-3-fluoro-1-(oxetan-3-yl)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1188];
N—((R)-3,3-difluoro-1-methylpiperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1189];
N—((R)-3,3-difluoro-1-(methyl-d₃)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1190];
1-((R)-3,3-difluoro-4-((5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one [1191];
1-((R)-3,3-difluoro-4-((5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1192];
1-((R)-3,3-difluoro-4-((5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1193];
N—((R)-3,3-difluoro-1-(methylsulfonyl)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1194];
N—((R)-3,3-difluoro-1-(oxetan-3-yl)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1195];
(S)-5-(1-(1-fluoropropan-2-yl)-1H-benzo[d][1,2,3]triazol-6-yl)-4-methoxy-N-(2-oxaspiro[3.5]nonan-7-yl)pyrrolo[2,1-f][1,2,4]triazin-2-amine [1196];
(R)-3-((5-(1-(1-fluoropropan-2-yl)-1H-benzo[d][1,2,3]triazol-6-yl)-4-methoxypyrrolo[2,1-f][1,2,4]triazin-2-yl)amino)-2,2-dimethylpropanenitrile [1197];
(1S,4s)-4-((5-(1-((R)-1-fluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1198];
(1R,4r)-4-((5-(1-((R)-1-fluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1199]; and
N—((R)-4,4-difluoro-1-methylpyrrolidin-3-yl)-5-(1-((R)-1-fluoropropan-2-yl)-1H-benzo[d][1,2,3]triazol-6-yl)-4-methoxypyrrolo[2,1-f][1,2,4]triazin-2-amine [1200]; or a pharmaceutically acceptable salt thereof.

29. The compound of claim 1, wherein the compound of Formula I is selected from the group consisting of
1-((R)-3,3-difluoro-4-((5-(1-((R)-1-fluoropropan-2-yl)-1H-benzo[d][1,2,3]triazol-6-yl)-4-methoxypyrrolo[2,1-f][1,2,4]triazin-2-yl)amino)pyrrolidin-1-yl)ethan-1-one [1201];
N—((R)-4,4-difluoro-1-(oxetan-3-yl)pyrrolidin-3-yl)-5-(1-((R)-1-fluoropropan-2-yl)-1H-benzo[d][1,2,3]triazol-6-yl)-4-methoxypyrrolo[2,1-f][1,2,4]triazin-2-amine [1202];
(R)-5-(1-(1-fluoropropan-2-yl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1203];
N-((3S,4R)-3-fluoro-1-methylpiperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1204];
N-((3R,4S)-3-fluoro-1-methylpiperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1205];
1-((3R,4S)-3-fluoro-4-((5-(1-((R)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one [1206];
1-((3R,4S)-3-fluoro-4-((5-(1-((R)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1207];
1-((3R,4S)-3-fluoro-4-((5-(1-((R)-1-fluoropropan-2-yl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1208];
N-((3S,4R)-3-fluoro-1-(oxetan-3-yl)piperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1209];
N-((3R,4R)-3-fluoro-1-(oxetan-3-yl)piperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1210];
N-((3S,4S)-3-fluoro-1-(oxetan-3-yl)piperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1211];
N-((3R,4S)-3-fluoro-1-(oxetan-3-yl)piperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1212];
N—((R)-3,3-difluoro-1-methylpiperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1213];
N—((R)-3,3-difluoro-1-(methyl-d₃)piperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1214];
1-((R)-3,3-difluoro-4-((5-(1-((R)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one [1215];
1-((R)-3,3-difluoro-4-((5-(1-((R)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1216];
1-((R)-3,3-difluoro-4-((5-(1-((R)-1-fluoropropan-2-yl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1217];
N—((R)-3,3-difluoro-1-(methylsulfonyl)piperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1218];
N—((R)-3,3-difluoro-1-(oxetan-3-yl)piperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1219];
(R)-5-(1-(1-fluoropropan-2-yl)-1H-benzo[d][1,2,3]triazol-6-yl)-4-methoxy-N-(2-oxaspiro[3.5]nonan-7-yl)pyrrolo[2,1-f][1,2,4]triazin-2-amine [1220];
(S)-3-((6-fluoro-5-(1-(1-fluoropropan-2-yl)-1H-benzo[d][1,2,3]triazol-6-yl)-4-methoxypyrrolo[2,1-f][1,2,4]triazin-2-yl)amino)-2,2-dimethylpropanenitrile [1221];
(1R,4s)-4-((6-fluoro-5-(1-((S)-1-fluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1222];
(1S,4r)-4-((6-fluoro-5-(1-((S)-1-fluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1223];
N—((R)-4,4-difluoro-1-methylpyrrolidin-3-yl)-6-fluoro-5-(1-((S)-1-fluoropropan-2-yl)-1H-benzo[d][1,2,3]triazol-6-yl)-4-methoxypyrrolo[2,1-f][1,2,4]triazin-2-amine [1224];
1-((R)-3,3-difluoro-4-((6-fluoro-5-(1-((S)-1-fluoropropan-2-yl)-1H-benzo[d][1,2,3]triazol-6-yl)-4-methoxypyrrolo[2,1-f][1,2,4]triazin-2-yl)amino)pyrrolidin-1-yl)ethan-1-one [1225];
N—((R)-4,4-difluoro-1-(oxetan-3-yl)pyrrolidin-3-yl)-6-fluoro-5-(1-((S)-1-fluoropropan-2-yl)-1H-benzo[d][1,2,3]triazol-6-yl)-4-methoxypyrrolo[2,1-f][1,2,4]triazin-2-amine [1226];
(S)-6-fluoro-5-(1-(1-fluoropropan-2-yl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1227];
6-fluoro-N-((3S,4R)-3-fluoro-1-methylpiperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1228];
6-fluoro-N-((3R,4S)-3-fluoro-1-methylpiperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1229];
1-((3R,4S)-3-fluoro-4-((6-fluoro-5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one [1230];
1-((3R,4S)-3-fluoro-4-((6-fluoro-5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1231];
1-((3R,4S)-3-fluoro-4-((6-fluoro-5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1232];
6-fluoro-N-((3S,4R)-3-fluoro-1-(oxetan-3-yl)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1233];
6-fluoro-N-((3R,4R)-3-fluoro-1-(oxetan-3-yl)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1234];
6-fluoro-N-((3S,4S)-3-fluoro-1-(oxetan-3-yl)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1235];
6-fluoro-N-((3R,4S)-3-fluoro-1-(oxetan-3-yl)piperidin-4-yl)-5-(1-((S)-1-fluoropropan-2-yl)-1H-benzo[d][1,2,3]triazol-6-yl)-4-methoxypyrrolo[2,1-f][1,2,4]triazin-2-amine [1236];
N—((R)-3,3-difluoro-1-methylpiperidin-4-yl)-6-fluoro-5-(1-((S)-1-fluoropropan-2-yl)-1H-benzo[d][1,2,3]triazol-6-yl)-4-methoxypyrrolo[2,1-f][1,2,4]triazin-2-amine [1237];
N—((R)-3,3-difluoro-1-(methyl-d₃)piperidin-4-yl)-6-fluoro-5-(1-((S)-1-fluoropropan-2-yl)-1H-benzo[d][1,2,3]triazol-6-yl)-4-methoxypyrrolo[2,1-f][1,2,4]triazin-2-amine [1238];
1-((R)-3,3-difluoro-4-((6-fluoro-5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one [1239];
1-((R)-3,3-difluoro-4-((6-fluoro-5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1240];
1-((R)-3,3-difluoro-4-((6-fluoro-5-(1-((S)-1-fluoropropan-2-yl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1241];
N—((R)-3,3-difluoro-1-(methylsulfonyl)piperidin-4-yl)-6-fluoro-5-(1-((S)-1-fluoropropan-2-yl)-1H-benzo[d][1,2,3]triazol-6-yl)-4-methoxypyrrolo[2,1-f][1,2,4]triazin-2-amine [1242];
N—((R)-3,3-difluoro-1-(oxetan-3-yl)piperidin-4-yl)-6-fluoro-5-(1-((S)-1-fluoropropan-2-yl)-1H-benzo[d][1,2,3]triazol-6-yl)-4-methoxypyrrolo[2,1-f][1,2,4]triazin-2-amine [1243];
(S)-6-fluoro-5-(1-(1-fluoropropan-2-yl)-1H-benzo[d][1,2,3]triazol-6-yl)-4-methoxy-N-(2-oxaspiro[3.5]nonan-7-yl)pyrrolo[2,1-f][1,2,4]triazin-2-amine [1244];
(R)-3-((6-fluoro-5-(1-(1-fluoropropan-2-yl)-1H-benzo[d][1,2,3]triazol-6-yl)-4-methoxypyrrolo[2,1-f][1,2,4]triazin-2-yl)amino)-2,2-dimethylpropanenitrile [1245];
(1S,4s)-4-((6-fluoro-5-(1-((R)-1-fluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1246];
(1R,4r)-4-((6-fluoro-5-(1-((R)-1-fluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1247];
N—((R)-4,4-difluoro-1-methylpyrrolidin-3-yl)-6-fluoro-5-(1-((R)-1-fluoropropan-2-yl)-1H-benzo[d][1,2,3]triazol-6-yl)-4-methoxypyrrolo[2,1-f][1,2,4]triazin-2-amine [1248];
1-((R)-3,3-difluoro-4-((6-fluoro-5-(1-((R)-1-fluoropropan-2-yl)-1H-benzo[d][1,2,3]triazol-6-yl)-4-methoxypyrrolo[2,1-f][1,2,4]triazin-2-yl)amino)pyrrolidin-1-yl)ethan-1-one [1249];
N—((R)-4,4-difluoro-1-(oxetan-3-yl)pyrrolidin-3-yl)-6-fluoro-5-(1-((R)-1-fluoropropan-2-yl)-1H-benzo[d][1,2,3]triazol-6-yl)-4-methoxypyrrolo[2,1-f][1,2,4]triazin-2-amine [1250];
(R)-6-fluoro-5-(1-(1-fluoropropan-2-yl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1251];
6-fluoro-N-((3S,4R)-3-fluoro-1-methylpiperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1252];
6-fluoro-N-((3R,4S)-3-fluoro-1-methylpiperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1253];
1-((3R,4S)-3-fluoro-4-((6-fluoro-5-(1-((R)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one [1254];
1-((3R,4S)-3-fluoro-4-((6-fluoro-5-(1-((R)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1255];
1-((3R,4S)-3-fluoro-4-((6-fluoro-5-(1-((R)-1-fluoropropan-2-yl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1256];
6-fluoro-N-((3S,4R)-3-fluoro-1-(oxetan-3-yl)piperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1257];
6-fluoro-N-((3R,4R)-3-fluoro-1-(oxetan-3-yl)piperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1258];
6-fluoro-N-((3S,4S)-3-fluoro-1-(oxetan-3-yl)piperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1259];
6-fluoro-N-((3R,4S)-3-fluoro-1-(oxetan-3-yl)piperidin-4-yl)-5-(1-((R)-1-fluoropropan-2-yl)-1H-benzo[d][1,2,3]triazol-6-yl)-4-methoxypyrrolo[2,1-f][1,2,4]triazin-2-amine [1260];
N—((R)-3,3-difluoro-1-methylpiperidin-4-yl)-6-fluoro-5-(1-((R)-1-fluoropropan-2-yl)-1H-benzo[d][1,2,3]triazol-6-yl)-4-methoxypyrrolo[2,1-f][1,2,4]triazin-2-amine [1261];
N—((R)-3,3-difluoro-1-(methyl-d₃)piperidin-4-yl)-6-fluoro-5-(1-((R)-1-fluoropropan-2-yl)-1H-benzo[d][1,2,3]triazol-6-yl)-4-methoxypyrrolo[2,1-f][1,2,4]triazin-2-amine [1262];
1-((R)-3,3-difluoro-4-((6-fluoro-5-(1-((R)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one [1263];
1-((R)-3,3-difluoro-4-((6-fluoro-5-(1-((R)-1-fluoropropan-2-yl)-1H-benzo[d][1,2,3]triazol-6-yl)-4-methoxypyrrolo[2,1-f][1,2,4]triazin-2-yl)amino)piperidin-1-yl)ethan-1-one-2,2,2-d₃ [1264];
1-((R)-3,3-difluoro-4-((6-fluoro-5-(1-((R)-1-fluoropropan-2-yl)-1H-benzo[d][1,2,3]triazol-6-yl)-4-methoxypyrrolo[2,1-f][1,2,4]triazin-2-yl)amino)piperidin-1-yl)-2-hydroxyethan-1-one [1265];
N—((R)-3,3-difluoro-1-(methylsulfonyl)piperidin-4-yl)-6-fluoro-5-(1-((R)-1-fluoropropan-2-yl)-1H-benzo[d][1,2,3]triazol-6-yl)-4-methoxypyrrolo[2,1-f][1,2,4]triazin-2-amine [1266];
N—((R)-3,3-difluoro-1-(oxetan-3-yl)piperidin-4-yl)-6-fluoro-5-(1-((R)-1-fluoropropan-2-yl)-1H-benzo[d][1,2,3]triazol-6-yl)-4-methoxypyrrolo[2,1-f][1,2,4]triazin-2-amine [1267];
(R)-6-fluoro-5-(1-(1-fluoropropan-2-yl)-1H-benzo[d][1,2,3]triazol-6-yl)-4-methoxy-N-(2-oxaspiro[3.5]nonan-7-yl)pyrrolo[2,1-f][1,2,4]triazin-2-amine [1268];
(1S,4r)-4-((5-(1-((S)-1-hydroxypropan-2-yl)-1H-benzo[d][1,2,3]triazol-6-yl)-4-methoxypyrrolo[2,1-f][1,2,4]triazin-2-yl)amino)-1-methylcyclohexan-1-ol [1269];
(S)-2-(6-(2-(((3R,4S)-3-fluoro-1-(oxetan-3-yl)piperidin-4-yl)amino)-4-methoxypyrrolo[2,1-f][1,2,4]triazin-5-yl)-1H-benzo[d][1,2,3]triazol-1-yl)propan-1-ol [1270];
1-((R)-3,3-difluoro-4-((5-(1-((S)-1-hydroxypropan-2-yl)-1H-benzo[d][1,2,3]triazol-6-yl)-4-methoxypyrrolo[2,1-f][1,2,4]triazin-2-yl)amino)piperidin-1-yl)ethan-1-one [1271];
(1R,4r)-4-((5-(1-((R)-1-hydroxypropan-2-yl)-1H-benzo[d][1,2,3]triazol-6-yl)-4-methoxypyrrolo[2,1-f][1,2,4]triazin-2-yl)amino)-1-methylcyclohexan-1-ol [1272];
(R)-2-(6-(2-(((3R,4S)-3-fluoro-1-(oxetan-3-yl)piperidin-4-yl)amino)-4-methoxypyrrolo[2,1-f][1,2,4]triazin-5-yl)-1H-benzo[d][1,2,3]triazol-1-yl)propan-1-ol [1273];
1-((R)-3,3-difluoro-4-((5-(1-((R)-1-hydroxypropan-2-yl)-1H-benzo[d][1,2,3]triazol-6-yl)-4-methoxypyrrolo[2,1-f][1,2,4]triazin-2-yl)amino)piperidin-1-yl)ethan-1-one [1274];
3-((5-(1-(3,3-difluoropropyl)-1H-benzo[d][1,2,3]triazol-6-yl)-4-methoxypyrrolo[2,1-f][1,2,4]triazin-2-yl)amino)-2,2-dimethylpropanenitrile [1275];
(1s,4s)-4-((5-(1-(3,3-difluoropropyl)-1H-benzo[d][1,2,3]triazol-6-yl)-4-methoxypyrrolo[2,1-f][1,2,4]triazin-2-yl)amino)-1-methylcyclohexan-1-ol [1276];
(1r,4r)-4-((5-(1-(3,3-difluoropropyl)-1H-benzo[d][1,2,3]triazol-6-yl)-4-methoxypyrrolo[2,1-f][1,2,4]triazin-2-yl)amino)-1-methylcyclohexan-1-ol [1277];
(R)—N-(4,4-difluoro-1-methylpyrrolidin-3-yl)-5-(1-(3,3-difluoropropyl)-1H-benzo[d][1,2,3]triazol-6-yl)-4-methoxypyrrolo[2,1-f][1,2,4]triazin-2-amine [1278];
(R)-1-(4-((5-(1-(3,3-difluoropropyl)-1H-benzo[d][1,2,3]triazol-6-yl)-4-methoxypyrrolo[2,1-f][1,2,4]triazin-2-yl)amino)-3,3-difluoropyrrolidin-1-yl)ethan-1-one [1279];
(R)—N-(4,4-difluoro-1-(oxetan-3-yl)pyrrolidin-3-yl)-5-(1-(3,3-difluoropropyl)-1H-benzo[d][1,2,3]triazol-6-yl)-4-methoxypyrrolo[2,1-f][1,2,4]triazin-2-amine [1280];
5-(1-(3,3-difluoropropyl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1281];
5-(1-(3,3-difluoropropyl)-1H-benzo[d][1,2,3]triazol-6-yl)-N-((3S,4R)-3-fluoro-1-methylpiperidin-4-yl)-4-methoxypyrrolo[2,1-f][1,2,4]triazin-2-amine [1282];
5-(1-(3,3-difluoropropyl)-1H-benzo[d][1,2,3]triazol-6-yl)-N-((3R,4S)-3-fluoro-1-methylpiperidin-4-yl)-4-methoxypyrrolo[2,1-f][1,2,4]triazin-2-amine [1283];
1-((3R,4S)-4-((5-(1-(3,3-difluoropropyl)-1H-benzo[d][1,2,3]triazol-6-yl)-4-methoxypyrrolo[2,1-f][1,2,4]triazin-2-yl)amino)-3-fluoropiperidin-1-yl)ethan-1-one [1284];
1-((3R,4S)-4-((5-(1-(3,3-difluoropropyl)-1H-benzo[d][1,2,3]triazol-6-yl)-4-methoxypyrrolo[2,1-f][1,2,4]triazin-2-yl)amino)-3-fluoropiperidin-1-yl)ethan-1-one-2,2,2-d₃ [1285];
1-((3R,4S)-4-((5-(1-(3,3-difluoropropyl)-1H-benzo[d][1,2,3]triazol-6-yl)-4-methoxypyrrolo[2,1-f][1,2,4]triazin-2-yl)amino)-3-fluoropiperidin-1-yl)-2-hydroxyethan-1-one [1286];
5-(1-(3,3-difluoropropyl)-1H-benzo[d][1,2,3]triazol-6-yl)-N-((3S,4R)-3-fluoro-1-(oxetan-3-yl)piperidin-4-yl)-4-methoxypyrrolo[2,1-f][1,2,4]triazin-2-amine [1287];
5-(1-(3,3-difluoropropyl)-1H-benzo[d][1,2,3]triazol-6-yl)-N-((3R,4R)-3-fluoro-1-(oxetan-3-yl)piperidin-4-yl)-4-methoxypyrrolo[2,1-f][1,2,4]triazin-2-amine [1288];
5-(1-(3,3-difluoropropyl)-1H-benzo[d][1,2,3]triazol-6-yl)-N-((3S,4S)-3-fluoro-1-(oxetan-3-yl)piperidin-4-yl)-4-methoxypyrrolo[2,1-f][1,2,4]triazin-2-amine [1289];
5-(1-(3,3-difluoropropyl)-1H-benzo[d][1,2,3]triazol-6-yl)-N-((3R,4S)-3-fluoro-1-(oxetan-3-yl)piperidin-4-yl)-4-methoxypyrrolo[2,1-f][1,2,4]triazin-2-amine [1290];
(R)—N-(3,3-difluoro-1-methylpiperidin-4-yl)-5-(1-(3,3-difluoropropyl)-1H-benzo[d][1,2,3]triazol-6-yl)-4-methoxypyrrolo[2,1-f][1,2,4]triazin-2-amine [1291];
(R)—N-(3,3-difluoro-1-(methyl-d₃)piperidin-4-yl)-5-(1-(3,3-difluoropropyl)-1H-benzo[d][1,2,3]triazol-6-yl)-4-methoxypyrrolo[2,1-f][1,2,4]triazin-2-amine [1292];
(R)-1-(4-((5-(1-(3,3-difluoropropyl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one [1293];
(R)-1-(4-((5-(1-(3,3-difluoropropyl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one-2,2,2-d₃ [1294];
(R)-1-(4-((5-(1-(3,3-difluoropropyl)-1H-benzo[d][1,2,3]triazol-6-yl)-4-methoxypyrrolo[2,1-f][1,2,4]triazin-2-yl)amino)-3,3-difluoropiperidin-1-yl)-2-hydroxyethan-1-one [1295];
(R)—N-(3,3-difluoro-1-(methylsulfonyl)piperidin-4-yl)-5-(1-(3,3-difluoropropyl)-1H-benzo[d][1,2,3]triazol-6-yl)-4-methoxypyrrolo[2,1-f][1,2,4]triazin-2-amine [1296];
(R)—N-(3,3-difluoro-1-(oxetan-3-yl)piperidin-4-yl)-5-(1-(3,3-difluoropropyl)-1H-benzo[d][1,2,3]triazol-6-yl)-4-methoxypyrrolo[2,1-f][1,2,4]triazin-2-amine [1297];
5-(1-(3,3-difluoropropyl)-1H-benzo[d][1,2,3]triazol-6-yl)-4-methoxy-N-(2-oxaspiro[3.5]nonan-7-yl)pyrrolo[2,1-f][1,2,4]triazin-2-amine [1298];
3-((5-(1-(3,3-difluoropropyl)-1H-benzo[d][1,2,3]triazol-6-yl)-6-fluoro-4-methoxypyrrolo[2,1-f][1,2,4]triazin-2-yl)amino)-2,2-dimethylpropanenitrile [1299]; and
(1s,4s)-4-((5-(1-(3,3-difluoropropyl)-1H-benzo[d][1,2,3]triazol-6-yl)-6-fluoro-4-methoxypyrrolo[2,1-f][1,2,4]triazin-2-yl)amino)-1-methylcyclohexan-1-ol[1300]; or a pharmaceutically acceptable salt thereof.

30. The compound of claim 1, wherein the compound of Formula I is selected from the group consisting of
(1r,4r)-4-((5-(1-(3,3-difluoropropyl)-1H-benzo[d][1,2,3]triazol-6-yl)-6-fluoro-4-methoxypyrrolo[2,1-f][1,2,4]triazin-2-yl)amino)-1-methylcyclohexan-1-ol [1301];
(R)—N-(4,4-difluoro-1-methylpyrrolidin-3-yl)-5-(1-(3,3-difluoropropyl)-1H-benzo[d][1,2,3]triazol-6-yl)-6-fluoro-4-methoxypyrrolo[2,1-f][1,2,4]triazin-2-amine [1302];
(R)-1-(4-((5-(1-(3,3-difluoropropyl)-1H-benzo[d][1,2,3]triazol-6-yl)-6-fluoro-4-methoxypyrrolo[2,1-f][1,2,4]triazin-2-yl)amino)-3,3-difluoropyrrolidin-1-yl)ethan-1-one [1303];
(R)—N-(4,4-difluoro-1-(oxetan-3-yl)pyrrolidin-3-yl)-5-(1-(3,3-difluoropropyl)-1H-benzo[d][1,2,3]triazol-6-yl)-6-fluoro-4-methoxypyrrolo[2,1-f][1,2,4]triazin-2-amine [1304];
5-(1-(3,3-difluoropropyl)-1H-benzo[d][1,2,3]triazol-6-yl)-6-fluoro-4-methoxy-N-(1-(oxetan-3-yl)piperidin-4-yl)pyrrolo[2,1-f][1,2,4]triazin-2-amine [1305];
5-(1-(3,3-difluoropropyl)-1H-benzo[d][1,2,3]triazol-6-yl)-6-fluoro-N-((3S,4R)-3-fluoro-1-methylpiperidin-4-yl)-4-methoxypyrrolo[2,1-f][1,2,4]triazin-2-amine [1306];
5-(1-(3,3-difluoropropyl)-1H-benzo[d][1,2,3]triazol-6-yl)-6-fluoro-N-((3R,4S)-3-fluoro-1-methylpiperidin-4-yl)-4-methoxypyrrolo[2,1-f][1,2,4]triazin-2-amine [1307];
1-((3R,4S)-4-((5-(1-(3,3-difluoropropyl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one [1308];
1-((3R,4S)-4-((5-(1-(3,3-difluoropropyl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one-2,2,2-d₃ [1309];
1-((3R,4S)-4-((5-(1-(3,3-difluoropropyl)-1H-benzo[d][1,2,3]triazol-6-yl)-6-fluoro-4-methoxypyrrolo[2,1-f][1,2,4]triazin-2-yl)amino)-3-fluoropiperidin-1-yl)-2-hydroxyethan-1-one [1310];
5-(1-(3,3-difluoropropyl)-1H-benzo[d][1,2,3]triazol-6-yl)-6-fluoro-N-((3S,4R)-3-fluoro-1-(oxetan-3-yl)piperidin-4-yl)-4-methoxypyrrolo[2,1-f][1,2,4]triazin-2-amine [1311];
5-(1-(3,3-difluoropropyl)-1H-benzo[d][1,2,3]triazol-6-yl)-6-fluoro-N-((3R,4R)-3-fluoro-1-(oxetan-3-yl)piperidin-4-yl)-4-methoxypyrrolo[2,1-f][1,2,4]triazin-2-amine [1312];
5-(1-(3,3-difluoropropyl)-1H-benzo[d][1,2,3]triazol-6-yl)-6-fluoro-N-((3S,4S)-3-fluoro-1-(oxetan-3-yl)piperidin-4-yl)-4-methoxypyrrolo[2,1-f][1,2,4]triazin-2-amine [1313];
5-(1-(3,3-difluoropropyl)-1H-benzo[d][1,2,3]triazol-6-yl)-6-fluoro-N-((3R,4S)-3-fluoro-1-(oxetan-3-yl)piperidin-4-yl)-4-methoxypyrrolo[2,1-f][1,2,4]triazin-2-amine [1314];
(R)—N-(3,3-difluoro-1-methylpiperidin-4-yl)-5-(1-(3,3-difluoropropyl)-1H-benzo[d][1,2,3]triazol-6-yl)-6-fluoro-4-methoxypyrrolo[2,1-f][1,2,4]triazin-2-amine [1315];
(R)—N-(3,3-difluoro-1-(methyl-d₃)piperidin-4-yl)-5-(1-(3,3-difluoropropyl)-1H-benzo[d][1,2,3]triazol-6-yl)-6-fluoro-4-methoxypyrrolo[2,1-f][1,2,4]triazin-2-amine [1316];
(R)-1-(4-((5-(1-(3,3-difluoropropyl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)ethan-1-one [1317];
(R)-1-(4-((5-(1-(3,3-difluoropropyl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)ethan-1-one-2,2,2-d₃ [1318];
(R)-1-(4-((5-(1-(3,3-difluoropropyl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)-2-hydroxyethan-1-one [1319];
(R)—N-(3,3-difluoro-1-(methylsulfonyl)piperidin-4-yl)-5-(1-(3,3-difluoropropyl)-1H-benzo[d][1,2,3]triazol-6-yl)-6-fluoro-4-methoxypyrrolo[2,1-f][1,2,4]triazin-2-amine [1320];
(R)—N-(3,3-difluoro-1-(oxetan-3-yl)piperidin-4-yl)-5-(1-(3,3-difluoropropyl)-1H-benzo[d][1,2,3]triazol-6-yl)-6-fluoro-4-methoxypyrrolo[2,1-f][1,2,4]triazin-2-amine [1321];
5-(1-(3,3-difluoropropyl)-1H-benzo[d][1,2,3]triazol-6-yl)-6-fluoro-4-methoxy-N-(2-oxaspiro[3.5]nonan-7-yl)pyrrolo[2,1-f][1,2,4]triazin-2-amine [1322];
3-((5-(1-(2,2-difluoropropyl)-1H-benzo[d][1,2,3]triazol-6-yl)-4-methoxypyrrolo[2,1-f][1,2,4]triazin-2-yl)amino)-2,2-dimethylpropanenitrile [1323];
(1s,4s)-4-((5-(1-(2,2-difluoropropyl)-1H-benzo[d][1,2,3]triazol-6-yl)-4-methoxypyrrolo[2,1-f][1,2,4]triazin-2-yl)amino)-1-methylcyclohexan-1-ol [1324];
(1r,4r)-4-((5-(1-(2,2-difluoropropyl)-1H-benzo[d][1,2,3]triazol-6-yl)-4-methoxypyrrolo[2,1-f][1,2,4]triazin-2-yl)amino)-1-methylcyclohexan-1-ol [1325];
(R)—N-(4,4-difluoro-1-methylpyrrolidin-3-yl)-5-(1-(2,2-difluoropropyl)-1H-benzo[d][1,2,3]triazol-6-yl)-4-methoxypyrrolo[2,1-f][1,2,4]triazin-2-amine [1326];
(R)-1-(4-((5-(1-(2,2-difluoropropyl)-1H-benzo[d][1,2,3]triazol-6-yl)-4-methoxypyrrolo[2,1-f][1,2,4]triazin-2-yl)amino)-3,3-difluoropyrrolidin-1-yl)ethan-1-one [1327];
(R)—N-(4,4-difluoro-1-(oxetan-3-yl)pyrrolidin-3-yl)-5-(1-(2,2-difluoropropyl)-1H-benzo[d][1,2,3]triazol-6-yl)-4-methoxypyrrolo[2,1-f][1,2,4]triazin-2-amine [1328];
5-(1-(2,2-difluoropropyl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1329];
5-(1-(2,2-difluoropropyl)-1H-benzo[d][1,2,3]triazol-6-yl)-N-((3S,4R)-3-fluoro-1-methylpiperidin-4-yl)-4-methoxypyrrolo[2,1-f][1,2,4]triazin-2-amine [1330];
5-(1-(2,2-difluoropropyl)-1H-benzo[d][1,2,3]triazol-6-yl)-N-((3R,4S)-3-fluoro-1-methylpiperidin-4-yl)-4-methoxypyrrolo[2,1-f][1,2,4]triazin-2-amine [1331];
1-((3R,4S)-4-((5-(1-(2,2-difluoropropyl)-1H-benzo[d][1,2,3]triazol-6-yl)-4-methoxypyrrolo[2,1-f][1,2,4]triazin-2-yl)amino)-3-fluoropiperidin-1-yl)ethan-1-one [1332];
1-((3R,4S)-4-((5-(1-(2,2-difluoropropyl)-1H-benzo[d][1,2,3]triazol-6-yl)-4-methoxypyrrolo[2,1-f][1,2,4]triazin-2-yl)amino)-3-fluoropiperidin-1-yl)ethan-1-one-2,2,2-d₃ [1333];
1-((3R,4S)-4-((5-(1-(2,2-difluoropropyl)-1H-benzo[d][1,2,3]triazol-6-yl)-4-methoxypyrrolo[2,1-f][1,2,4]triazin-2-yl)amino)-3-fluoropiperidin-1-yl)-2-hydroxyethan-1-one [1334];
5-(1-(2,2-difluoropropyl)-1H-benzo[d][1,2,3]triazol-6-yl)-N-((3S,4R)-3-fluoro-1-(oxetan-3-yl)piperidin-4-yl)-4-methoxypyrrolo[2,1-f][1,2,4]triazin-2-amine [1335];
5-(1-(2,2-difluoropropyl)-1H-benzo[d][1,2,3]triazol-6-yl)-N-((3R,4R)-3-fluoro-1-(oxetan-3-yl)piperidin-4-yl)-4-methoxypyrrolo[2,1-f][1,2,4]triazin-2-amine [1336];
5-(1-(2,2-difluoropropyl)-1H-benzo[d][1,2,3]triazol-6-yl)-N-((3S,4S)-3-fluoro-1-(oxetan-3-yl)piperidin-4-yl)-4-methoxypyrrolo[2,1-f][1,2,4]triazin-2-amine [1337];
5-(1-(2,2-difluoropropyl)-1H-benzo[d][1,2,3]triazol-6-yl)-N-((3R,4S)-3-fluoro-1-(oxetan-3-yl)piperidin-4-yl)-4-methoxypyrrolo[2,1-f][1,2,4]triazin-2-amine [1338];
(R)—N-(3,3-difluoro-1-methylpiperidin-4-yl)-5-(1-(2,2-difluoropropyl)-1H-benzo[d][1,2,3]triazol-6-yl)-4-methoxypyrrolo[2,1-f][1,2,4]triazin-2-amine [1339];
(R)—N-(3,3-difluoro-1-(methyl-d₃)piperidin-4-yl)-5-(1-(2,2-difluoropropyl)-1H-benzo[d][1,2,3]triazol-6-yl)-4-methoxypyrrolo[2,1-f][1,2,4]triazin-2-amine [1340];
(R)-1-(4-((5-(1-(2,2-difluoropropyl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one [1341];
(R)-1-(4-((5-(1-(2,2-difluoropropyl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one-2,2,2-d₃ [1342];
(R)-1-(4-((5-(1-(2,2-difluoropropyl)-1H-benzo[d][1,2,3]triazol-6-yl)-4-methoxypyrrolo[2,1-f][1,2,4]triazin-2-yl)amino)-3,3-difluoropiperidin-1-yl)-2-hydroxyethan-1-one [1343];
(R)—N-(3,3-difluoro-1-(methylsulfonyl)piperidin-4-yl)-5-(1-(2,2-difluoropropyl)-1H-benzo[d][1,2,3]triazol-6-yl)-4-methoxypyrrolo[2,1-f][1,2,4]triazin-2-amine [1344];
(R)—N-(3,3-difluoro-1-(oxetan-3-yl)piperidin-4-yl)-5-(1-(2,2-difluoropropyl)-1H-benzo[d][1,2,3]triazol-6-yl)-4-methoxypyrrolo[2,1-f][1,2,4]triazin-2-amine [1345];
5-(1-(2,2-difluoropropyl)-1H-benzo[d][1,2,3]triazol-6-yl)-4-methoxy-N-(2-oxaspiro[3.5]nonan-7-yl)pyrrolo[2,1-f][1,2,4]triazin-2-amine [1346];
3-((5-(1-(2,2-difluoropropyl)-1H-benzo[d][1,2,3]triazol-6-yl)-6-fluoro-4-methoxypyrrolo[2,1-f][1,2,4]triazin-2-yl)amino)-2,2-dimethylpropanenitrile [1347];
(1s,4s)-4-((5-(1-(2,2-difluoropropyl)-1H-benzo[d][1,2,3]triazol-6-yl)-6-fluoro-4-methoxypyrrolo[2,1-f][1,2,4]triazin-2-yl)amino)-1-methylcyclohexan-1-ol [1348];
(1r,4r)-4-((5-(1-(2,2-difluoropropyl)-1H-benzo[d][1,2,3]triazol-6-yl)-6-fluoro-4-methoxypyrrolo[2,1-f][1,2,4]triazin-2-yl)amino)-1-methylcyclohexan-1-ol [1349];
(R)—N-(4,4-difluoro-1-methylpyrrolidin-3-yl)-5-(1-(2,2-difluoropropyl)-1H-benzo[d][1,2,3]triazol-6-yl)-6-fluoro-4-methoxypyrrolo[2,1-f][1,2,4]triazin-2-amine [1350];
(R)-1-(4-((5-(1-(2,2-difluoropropyl)-1H-benzo[d][1,2,3]triazol-6-yl)-6-fluoro-4-methoxypyrrolo[2,1-f][1,2,4]triazin-2-yl)amino)-3,3-difluoropyrrolidin-1-yl)ethan-1-one [1351];
(R)—N-(4,4-difluoro-1-(oxetan-3-yl)pyrrolidin-3-yl)-5-(1-(2,2-difluoropropyl)-1H-benzo[d][1,2,3]triazol-6-yl)-6-fluoro-4-methoxypyrrolo[2,1-f][1,2,4]triazin-2-amine [1352];
5-(1-(2,2-difluoropropyl)-1H-benzo[d][1,2,3]triazol-6-yl)-6-fluoro-4-methoxy-N-(1-(oxetan-3-yl)piperidin-4-yl)pyrrolo[2,1-f][1,2,4]triazin-2-amine [1353];
5-(1-(2,2-difluoropropyl)-1H-benzo[d][1,2,3]triazol-6-yl)-6-fluoro-N-((3S,4R)-3-fluoro-1-methylpiperidin-4-yl)-4-methoxypyrrolo[2,1-f][1,2,4]triazin-2-amine [1354];
5-(1-(2,2-difluoropropyl)-1H-benzo[d][1,2,3]triazol-6-yl)-6-fluoro-N-((3R,4S)-3-fluoro-1-methylpiperidin-4-yl)-4-methoxypyrrolo[2,1-f][1,2,4]triazin-2-amine [1355];
1-((3R,4S)-4-((5-(1-(2,2-difluoropropyl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one [1356];
1-((3R,4S)-4-((5-(1-(2,2-difluoropropyl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one-2,2,2-d₃ [1357];
1-((3R,4S)-4-((5-(1-(2,2-difluoropropyl)-1H-benzo[d][1,2,3]triazol-6-yl)-6-fluoro-4-methoxypyrrolo[2,1-f][1,2,4]triazin-2-yl)amino)-3-fluoropiperidin-1-yl)-2-hydroxyethan-1-one [1358];
5-(1-(2,2-difluoropropyl)-1H-benzo[d][1,2,3]triazol-6-yl)-6-fluoro-N-((3S,4R)-3-fluoro-1-(oxetan-3-yl)piperidin-4-yl)-4-methoxypyrrolo[2,1-f][1,2,4]triazin-2-amine [1359];
5-(1-(2,2-difluoropropyl)-1H-benzo[d][1,2,3]triazol-6-yl)-6-fluoro-N-((3R,4R)-3-fluoro-1-(oxetan-3-yl)piperidin-4-yl)-4-methoxypyrrolo[2,1-f][1,2,4]triazin-2-amine [1360];
5-(1-(2,2-difluoropropyl)-1H-benzo[d][1,2,3]triazol-6-yl)-6-fluoro-N-((3S,4S)-3-fluoro-1-(oxetan-3-yl)piperidin-4-yl)-4-methoxypyrrolo[2,1-f][1,2,4]triazin-2-amine [1361];
5-(1-(2,2-difluoropropyl)-1H-benzo[d][1,2,3]triazol-6-yl)-6-fluoro-N-((3R,4S)-3-fluoro-1-(oxetan-3-yl)piperidin-4-yl)-4-methoxypyrrolo[2,1-f][1,2,4]triazin-2-amine [1362];
(R)—N-(3,3-difluoro-1-methylpiperidin-4-yl)-5-(1-(2,2-difluoropropyl)-1H-benzo[d][1,2,3]triazol-6-yl)-6-fluoro-4-methoxypyrrolo[2,1-f][1,2,4]triazin-2-amine [1363];
(R)—N-(3,3-difluoro-1-(methyl-d₃)piperidin-4-yl)-5-(1-(2,2-difluoropropyl)-1H-benzo[d][1,2,3]triazol-6-yl)-6-fluoro-4-methoxypyrrolo[2,1-f][1,2,4]triazin-2-amine [1364];
(R)-1-(4-((5-(1-(2,2-difluoropropyl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)ethan-1-one [1365];
(R)-1-(4-((5-(1-(2,2-difluoropropyl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)ethan-1-one-2,2,2-d₃ [1366];
(R)-1-(4-((5-(1-(2,2-difluoropropyl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)-2-hydroxyethan-1-one [1367];
(R)—N-(3,3-difluoro-1-(methylsulfonyl)piperidin-4-yl)-5-(1-(2,2-difluoropropyl)-1H-benzo[d][1,2,3]triazol-6-yl)-6-fluoro-4-methoxypyrrolo[2,1-f][1,2,4]triazin-2-amine [1368];
(R)—N-(3,3-difluoro-1-(oxetan-3-yl)piperidin-4-yl)-5-(1-(2,2-difluoropropyl)-1H-benzo[d][1,2,3]triazol-6-yl)-6-fluoro-4-methoxypyrrolo[2,1-f][1,2,4]triazin-2-amine [1369];
5-(1-(2,2-difluoropropyl)-1H-benzo[d][1,2,3]triazol-6-yl)-6-fluoro-4-methoxy-N-(2-oxaspiro[3.5]nonan-7-yl)pyrrolo[2,1-f][1,2,4]triazin-2-amine [1370];
(S)-3-((5-(1-(1,1-difluoropropan-2-yl)-1H-benzo[d][1,2,3]triazol-6-yl)-4-methoxypyrrolo[2,1-f][1,2,4]triazin-2-yl)amino)-2,2-dimethylpropanenitrile [1371];
(1R,4s)-4-((5-(1-((S)-1,1-difluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1372];
(1S,4r)-4-((5-(1-((S)-1,1-difluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1373];
N—((R)-4,4-difluoro-1-methylpyrrolidin-3-yl)-5-(1-((S)-1,1-difluoropropan-2-yl)-1H-benzo[d][1,2,3]triazol-6-yl)-4-methoxypyrrolo[2,1-f][1,2,4]triazin-2-amine [1374];
1-((R)-4-((5-(1-((S)-1,1-difluoropropan-2-yl)-1H-benzo[d][1,2,3]triazol-6-yl)-4-methoxypyrrolo[2,1-f][1,2,4]triazin-2-yl)amino)-3,3-difluoropyrrolidin-1-yl)ethan-1-one [1375];
N—((R)-4,4-difluoro-1-(oxetan-3-yl)pyrrolidin-3-yl)-5-(1-((S)-1,1-difluoropropan-2-yl)-1H-benzo[d][1,2,3]triazol-6-yl)-4-methoxypyrrolo[2,1-f][1,2,4]triazin-2-amine [1376];
(S)-5-(1-(1,1-difluoropropan-2-yl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1377];
5-(1-((S)-1,1-difluoropropan-2-yl)-1H-benzo[d][1,2,3]triazol-6-yl)-N-((3S,4R)-3-fluoro-1-methylpiperidin-4-yl)-4-methoxypyrrolo[2,1-f][1,2,4]triazin-2-amine [1378];
5-(1-((S)-1,1-difluoropropan-2-yl)-1H-benzo[d][1,2,3]triazol-6-yl)-N-((3R,4S)-3-fluoro-1-methylpiperidin-4-yl)-4-methoxypyrrolo[2,1-f][1,2,4]triazin-2-amine [1379];
1-((3R,4S)-4-((5-(1-((S)-1,1-difluoropropan-2-yl)-1H-benzo[d][1,2,3]triazol-6-yl)-4-methoxypyrrolo[2,1-f][1,2,4]triazin-2-yl)amino)-3-fluoropiperidin-1-yl)ethan-1-one [1380];
1-((3R,4S)-4-((5-(1-((S)-1,1-difluoropropan-2-yl)-1H-benzo[d][1,2,3]triazol-6-yl)-4-methoxypyrrolo[2,1-f][1,2,4]triazin-2-yl)amino)-3-fluoropiperidin-1-yl)ethan-1-one-2,2,2-d₃ [1381];
1-((3R,4S)-4-((5-(1-((S)-1,1-difluoropropan-2-yl)-1H-benzo[d][1,2,3]triazol-6-yl)-4-methoxypyrrolo[2,1-f][1,2,4]triazin-2-yl)amino)-3-fluoropiperidin-1-yl)-2-hydroxyethan-1-one [1382];
5-(1-((S)-1,1-difluoropropan-2-yl)-1H-benzo[d][1,2,3]triazol-6-yl)-N-((3S,4R)-3-fluoro-1-(oxetan-3-yl)piperidin-4-yl)-4-methoxypyrrolo[2,1-f][1,2,4]triazin-2-amine [1383];
5-(1-((S)-1,1-difluoropropan-2-yl)-1H-benzo[d][1,2,3]triazol-6-yl)-N-((3R,4R)-3-fluoro-1-(oxetan-3-yl)piperidin-4-yl)-4-methoxypyrrolo[2,1-f][1,2,4]triazin-2-amine [1384];
5-(1-((S)-1,1-difluoropropan-2-yl)-1H-benzo[d][1,2,3]triazol-6-yl)-N-((3S,4S)-3-fluoro-1-(oxetan-3-yl)piperidin-4-yl)-4-methoxypyrrolo[2,1-f][1,2,4]triazin-2-amine [1385];
5-(1-((S)-1,1-difluoropropan-2-yl)-1H-benzo[d][1,2,3]triazol-6-yl)-N-((3R,4S)-3-fluoro-1-(oxetan-3-yl)piperidin-4-yl)-4-methoxypyrrolo[2,1-f][1,2,4]triazin-2-amine [1386];
N—((R)-3,3-difluoro-1-methylpiperidin-4-yl)-5-(1-((S)-1,1-difluoropropan-2-yl)-1H-benzo[d][1,2,3]triazol-6-yl)-4-methoxypyrrolo[2,1-f][1,2,4]triazin-2-amine [1387];
N—((R)-3,3-difluoro-1-(methyl-d₃)piperidin-4-yl)-5-(1-((S)-1,1-difluoropropan-2-yl)-1H-benzo[d][1,2,3]triazol-6-yl)-4-methoxypyrrolo[2,1-f][1,2,4]triazin-2-amine [1388];
1-((R)-4-((5-(1-((S)-1,1-difluoropropan-2-yl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one [1389];
1-((R)-4-((5-(1-((S)-1,1-difluoropropan-2-yl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one-2,2,2-d₃ [1390];
1-((R)-4-((5-(1-((S)-1,1-difluoropropan-2-yl)-1H-benzo[d][1,2,3]triazol-6-yl)-4-methoxypyrrolo[2,1-f][1,2,4]triazin-2-yl)amino)-3,3-difluoropiperidin-1-yl)-2-hydroxyethan-1-one [1391];
N—((R)-3,3-difluoro-1-(methylsulfonyl)piperidin-4-yl)-5-(1-((S)-1,1-difluoropropan-2-yl)-1H-benzo[d][1,2,3]triazol-6-yl)-4-methoxypyrrolo[2,1-f][1,2,4]triazin-2-amine [1392];
N—((R)-3,3-difluoro-1-(oxetan-3-yl)piperidin-4-yl)-5-(1-((S)-1,1-difluoropropan-2-yl)-1H-benzo[d][1,2,3]triazol-6-yl)-4-methoxypyrrolo[2,1-f][1,2,4]triazin-2-amine [1393];
(S)-5-(1-(1,1-difluoropropan-2-yl)-1H-benzo[d][1,2,3]triazol-6-yl)-4-methoxy-N-(2-oxaspiro[3.5]nonan-7-yl)pyrrolo[2,1-f][1,2,4]triazin-2-amine [1394];
(S)-3-((5-(1-(1,1-difluoropropan-2-yl)-1H-benzo[d][1,2,3]triazol-6-yl)-6-fluoro-4-methoxypyrrolo[2,1-f][1,2,4]triazin-2-yl)amino)-2,2-dimethylpropanenitrile [1395];
(1R,4s)-4-((5-(1-((S)-1,1-difluoropropan-2-yl)-1H-benzo[d][1,2,3]triazol-6-yl)-6-fluoro-4-methoxypyrrolo[2,1-f][1,2,4]triazin-2-yl)amino)-1-methylcyclohexan-1-ol [1396];
(1S,4r)-4-((5-(1-((S)-1,1-difluoropropan-2-yl)-1H-benzo[d][1,2,3]triazol-6-yl)-6-fluoro-4-methoxypyrrolo[2,1-f][1,2,4]triazin-2-yl)amino)-1-methylcyclohexan-1-ol [1397];
N—((R)-4,4-difluoro-1-methylpyrrolidin-3-yl)-5-(1-((S)-1,1-difluoropropan-2-yl)-1H-benzo[d][1,2,3]triazol-6-yl)-6-fluoro-4-methoxypyrrolo[2,1-f][1,2,4]triazin-2-amine [1398];
1-((R)-4-((5-(1-((S)-1,1-difluoropropan-2-yl)-1H-benzo[d][1,2,3]triazol-6-yl)-6-fluoro-4-methoxypyrrolo[2,1-f][1,2,4]triazin-2-yl)amino)-3,3-difluoropyrrolidin-1-yl)ethan-1-one [1399]; and
N—((R)-4,4-difluoro-1-(oxetan-3-yl)pyrrolidin-3-yl)-5-(1-((S)-1,1-difluoropropan-2-yl)-1H-benzo[d][1,2,3]triazol-6-yl)-6-fluoro-4-methoxypyrrolo[2,1-f][1,2,4]triazin-2-amine [1400]; or a pharmaceutically acceptable salt thereof.

31. The compound of claim 1, wherein the compound of Formula I is selected from the group consisting of
(S)-5-(1-(1,1-difluoropropan-2-yl)-1H-benzo[d][1,2,3]triazol-6-yl)-6-fluoro-4-methoxy-N-(1-(oxetan-3-yl)piperidin-4-yl)pyrrolo[2,1-f][1,2,4]triazin-2-amine [1401];
5-(1-((S)-1,1-difluoropropan-2-yl)-1H-benzo[d][1,2,3]triazol-6-yl)-6-fluoro-N-((3S,4R)-3-fluoro-1-methylpiperidin-4-yl)-4-methoxypyrrolo[2,1-f][1,2,4]triazin-2-amine [1402];
5-(1-((S)-1,1-difluoropropan-2-yl)-1H-benzo[d][1,2,3]triazol-6-yl)-6-fluoro-N-((3R,4S)-3-fluoro-1-methylpiperidin-4-yl)-4-methoxypyrrolo[2,1-f][1,2,4]triazin-2-amine [1403];
1-((3R,4S)-4-((5-(1-((S)-1,1-difluoropropan-2-yl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one [1404];
1-((3R,4S)-4-((5-(1-((S)-1,1-difluoropropan-2-yl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one-2,2,2-d₃ [1405];
1-((3R,4S)-4-((5-(1-((S)-1,1-difluoropropan-2-yl)-1H-benzo[d][1,2,3]triazol-6-yl)-6-fluoro-4-methoxypyrrolo[2,1-f][1,2,4]triazin-2-yl)amino)-3-fluoropiperidin-1-yl)-2-hydroxyethan-1-one [1406];
5-(1-((S)-1,1-difluoropropan-2-yl)-1H-benzo[d][1,2,3]triazol-6-yl)-6-fluoro-N-((3S,4R)-3-fluoro-1-(oxetan-3-yl)piperidin-4-yl)-4-methoxypyrrolo[2,1-f][1,2,4]triazin-2-amine [1407];
5-(1-((S)-1,1-difluoropropan-2-yl)-1H-benzo[d][1,2,3]triazol-6-yl)-6-fluoro-N-((3R,4R)-3-fluoro-1-(oxetan-3-yl)piperidin-4-yl)-4-methoxypyrrolo[2,1-f][1,2,4]triazin-2-amine [1408];
5-(1-((S)-1,1-difluoropropan-2-yl)-1H-benzo[d][1,2,3]triazol-6-yl)-6-fluoro-N-((3S,4S)-3-fluoro-1-(oxetan-3-yl)piperidin-4-yl)-4-methoxypyrrolo[2,1-f][1,2,4]triazin-2-amine [1409];
5-(1-((S)-1,1-difluoropropan-2-yl)-1H-benzo[d][1,2,3]triazol-6-yl)-6-fluoro-N-((3R,4S)-3-fluoro-1-(oxetan-3-yl)piperidin-4-yl)-4-methoxypyrrolo[2,1-f][1,2,4]triazin-2-amine [1410];
N—((R)-3,3-difluoro-1-methylpiperidin-4-yl)-5-(1-((S)-1,1-difluoropropan-2-yl)-1H-benzo[d][1,2,3]triazol-6-yl)-6-fluoro-4-methoxypyrrolo[2,1-f][1,2,4]triazin-2-amine [1411];
N—((R)-3,3-difluoro-1-(methyl-d₃)piperidin-4-yl)-5-(1-((S)-1,1-difluoropropan-2-yl)-1H-benzo[d][1,2,3]triazol-6-yl)-6-fluoro-4-methoxypyrrolo[2,1-f][1,2,4]triazin-2-amine [1412];
1-((R)-4-((5-(1-((S)-1,1-difluoropropan-2-yl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)ethan-1-one [1413];
1-((R)-4-((5-(1-((S)-1,1-difluoropropan-2-yl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)ethan-1-one-2,2,2-d₃ [1414];
1-((R)-4-((5-(1-((S)-1,1-difluoropropan-2-yl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)-2-hydroxyethan-1-one [1415];
N—((R)-3,3-difluoro-1-(methylsulfonyl)piperidin-4-yl)-5-(1-((S)-1,1-difluoropropan-2-yl)-1H-benzo[d][1,2,3]triazol-6-yl)-6-fluoro-4-methoxypyrrolo[2,1-f][1,2,4]triazin-2-amine [1416];
N—((R)-3,3-difluoro-1-(oxetan-3-yl)piperidin-4-yl)-5-(1-((S)-1,1-difluoropropan-2-yl)-1H-benzo[d][1,2,3]triazol-6-yl)-6-fluoro-4-methoxypyrrolo[2,1-f][1,2,4]triazin-2-amine [1417];
(S)-5-(1-(1,1-difluoropropan-2-yl)-1H-benzo[d][1,2,3]triazol-6-yl)-6-fluoro-4-methoxy-N-(2-oxaspiro[3.5]nonan-7-yl)pyrrolo[2,1-f][1,2,4]triazin-2-amine [1418];
(R)-3-((5-(1-(1,1-difluoropropan-2-yl)-1H-benzo[d][1,2,3]triazol-6-yl)-4-methoxypyrrolo[2,1-f][1,2,4]triazin-2-yl)amino)-2,2-dimethylpropanenitrile [1419];
(1S,4s)-4-((5-(1-((R)-1,1-difluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1420];
(1R,4r)-4-((5-(1-((R)-1,1-difluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1421];
N—((R)-4,4-difluoro-1-methylpyrrolidin-3-yl)-5-(1-((R)-1,1-difluoropropan-2-yl)-1H-benzo[d][1,2,3]triazol-6-yl)-4-methoxypyrrolo[2,1-f][1,2,4]triazin-2-amine [1422];
1-((R)-4-((5-(1-((R)-1,1-difluoropropan-2-yl)-1H-benzo[d][1,2,3]triazol-6-yl)-4-methoxypyrrolo[2,1-f][1,2,4]triazin-2-yl)amino)-3,3-difluoropyrrolidin-1-yl)ethan-1-one [1423];
N—((R)-4,4-difluoro-1-(oxetan-3-yl)pyrrolidin-3-yl)-5-(1-((R)-1,1-difluoropropan-2-yl)-1H-benzo[d][1,2,3]triazol-6-yl)-4-methoxypyrrolo[2,1-f][1,2,4]triazin-2-amine [1424];
(R)-5-(1-(1,1-difluoropropan-2-yl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1425];
5-(1-((R)-1,1-difluoropropan-2-yl)-1H-benzo[d][1,2,3]triazol-6-yl)-N-((3S,4R)-3-fluoro-1-methylpiperidin-4-yl)-4-methoxypyrrolo[2,1-f][1,2,4]triazin-2-amine [1426];
5-(1-((R)-1,1-difluoropropan-2-yl)-1H-benzo[d][1,2,3]triazol-6-yl)-N-((3R,4S)-3-fluoro-1-methylpiperidin-4-yl)-4-methoxypyrrolo[2,1-f][1,2,4]triazin-2-amine [1427];
1-((3R,4S)-4-((5-(1-((R)-1,1-difluoropropan-2-yl)-1H-benzo[d][1,2,3]triazol-6-yl)-4-methoxypyrrolo[2,1-f][1,2,4]triazin-2-yl)amino)-3-fluoropiperidin-1-yl)ethan-1-one [1428];
1-((3R,4S)-4-((5-(1-((R)-1,1-difluoropropan-2-yl)-1H-benzo[d][1,2,3]triazol-6-yl)-4-methoxypyrrolo[2,1-f][1,2,4]triazin-2-yl)amino)-3-fluoropiperidin-1-yl)ethan-1-one-2,2,2-d₃ [1429];
1-((3R,4S)-4-((5-(1-((R)-1,1-difluoropropan-2-yl)-1H-benzo[d][1,2,3]triazol-6-yl)-4-methoxypyrrolo[2,1-f][1,2,4]triazin-2-yl)amino)-3-fluoropiperidin-1-yl)-2-hydroxyethan-1-one [1430];
5-(1-((R)-1,1-difluoropropan-2-yl)-1H-benzo[d][1,2,3]triazol-6-yl)-N-((3S,4R)-3-fluoro-1-(oxetan-3-yl)piperidin-4-yl)-4-methoxypyrrolo[2,1-f][1,2,4]triazin-2-amine [1431];
5-(1-((R)-1,1-difluoropropan-2-yl)-1H-benzo[d][1,2,3]triazol-6-yl)-N-((3R,4R)-3-fluoro-1-(oxetan-3-yl)piperidin-4-yl)-4-methoxypyrrolo[2,1-f][1,2,4]triazin-2-amine [1432];
5-(1-((R)-1,1-difluoropropan-2-yl)-1H-benzo[d][1,2,3]triazol-6-yl)-N-((3S,4S)-3-fluoro-1-(oxetan-3-yl)piperidin-4-yl)-4-methoxypyrrolo[2,1-f][1,2,4]triazin-2-amine [1433];
5-(1-((R)-1,1-difluoropropan-2-yl)-1H-benzo[d][1,2,3]triazol-6-yl)-N-((3R,4S)-3-fluoro-1-(oxetan-3-yl)piperidin-4-yl)-4-methoxypyrrolo[2,1-f][1,2,4]triazin-2-amine [1434];
N—((R)-3,3-difluoro-1-methylpiperidin-4-yl)-5-(1-((R)-1,1-difluoropropan-2-yl)-1H-benzo[d][1,2,3]triazol-6-yl)-4-methoxypyrrolo[2,1-f][1,2,4]triazin-2-amine [1435];
N—((R)-3,3-difluoro-1-(methyl-d₃)piperidin-4-yl)-5-(1-((R)-1,1-difluoropropan-2-yl)-1H-benzo[d][1,2,3]triazol-6-yl)-4-methoxypyrrolo[2,1-f][1,2,4]triazin-2-amine [1436];
1-((R)-4-((5-(1-((R)-1,1-difluoropropan-2-yl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one [1437];
1-((R)-4-((5-(1-((R)-1,1-difluoropropan-2-yl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one-2,2,2-d₃ [1438];
1-((R)-4-((5-(1-((R)-1,1-difluoropropan-2-yl)-1H-benzo[d][1,2,3]triazol-6-yl)-4-methoxypyrrolo[2,1-f][1,2,4]triazin-2-yl)amino)-3,3-difluoropiperidin-1-yl)-2-hydroxyethan-1-one [1439];
N—((R)-3,3-difluoro-1-(methylsulfonyl)piperidin-4-yl)-5-(1-((R)-1,1-difluoropropan-2-yl)-1H-benzo[d][1,2,3]triazol-6-yl)-4-methoxypyrrolo[2,1-f][1,2,4]triazin-2-amine [1440];
N—((R)-3,3-difluoro-1-(oxetan-3-yl)piperidin-4-yl)-5-(1-((R)-1,1-difluoropropan-2-yl)-1H-benzo[d][1,2,3]triazol-6-yl)-4-methoxypyrrolo[2,1-f][1,2,4]triazin-2-amine [1441];
(R)-5-(1-(1,1-difluoropropan-2-yl)-1H-benzo[d][1,2,3]triazol-6-yl)-4-methoxy-N-(2-oxaspiro[3.5]nonan-7-yl)pyrrolo[2,1-f][1,2,4]triazin-2-amine [1442];
(R)-3-((5-(1-(1,1-difluoropropan-2-yl)-1H-benzo[d][1,2,3]triazol-6-yl)-6-fluoro-4-methoxypyrrolo[2,1-f][1,2,4]triazin-2-yl)amino)-2,2-dimethylpropanenitrile [1443];
(1S,4s)-4-((5-(1-((R)-1,1-difluoropropan-2-yl)-1H-benzo[d][1,2,3]triazol-6-yl)-6-fluoro-4-methoxypyrrolo[2,1-f][1,2,4]triazin-2-yl)amino)-1-methylcyclohexan-1-ol [1444];
(1R,4r)-4-((5-(1-((R)-1,1-difluoropropan-2-yl)-1H-benzo[d][1,2,3]triazol-6-yl)-6-fluoro-4-methoxypyrrolo[2,1-f][1,2,4]triazin-2-yl)amino)-1-methylcyclohexan-1-ol [1445];
N—((R)-4,4-difluoro-1-methylpyrrolidin-3-yl)-5-(1-((R)-1,1-difluoropropan-2-yl)-1H-benzo[d][1,2,3]triazol-6-yl)-6-fluoro-4-methoxypyrrolo[2,1-f][1,2,4]triazin-2-amine [1446];
1-((R)-4-((5-(1-((R)-1,1-difluoropropan-2-yl)-1H-benzo[d][1,2,3]triazol-6-yl)-6-fluoro-4-methoxypyrrolo[2,1-f][1,2,4]triazin-2-yl)amino)-3,3-difluoropyrrolidin-1-yl)ethan-1-one [1447];
N—((R)-4,4-difluoro-1-(oxetan-3-yl)pyrrolidin-3-yl)-5-(1-((R)-1,1-difluoropropan-2-yl)-1H-benzo[d][1,2,3]triazol-6-yl)-6-fluoro-4-methoxypyrrolo[2,1-f][1,2,4]triazin-2-amine [1448];
(R)-5-(1-(1,1-difluoropropan-2-yl)-1H-benzo[d][1,2,3]triazol-6-yl)-6-fluoro-4-methoxy-N-(1-(oxetan-3-yl)piperidin-4-yl)pyrrolo[2,1-f][1,2,4]triazin-2-amine [1449];
5-(1-((R)-1,1-difluoropropan-2-yl)-1H-benzo[d][1,2,3]triazol-6-yl)-6-fluoro-N-((3S,4R)-3-fluoro-1-methylpiperidin-4-yl)-4-methoxypyrrolo[2,1-f][1,2,4]triazin-2-amine [1450];
5-(1-((R)-1,1-difluoropropan-2-yl)-1H-benzo[d][1,2,3]triazol-6-yl)-6-fluoro-N-((3R,4S)-3-fluoro-1-methylpiperidin-4-yl)-4-methoxypyrrolo[2,1-f][1,2,4]triazin-2-amine [1451];
1-((3R,4S)-4-((5-(1-((R)-1,1-difluoropropan-2-yl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one [1452];
1-((3R,4S)-4-((5-(1-((R)-1,1-difluoropropan-2-yl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one-2,2,2-d₃ [1453];
1-((3R,4S)-4-((5-(1-((R)-1,1-difluoropropan-2-yl)-1H-benzo[d][1,2,3]triazol-6-yl)-6-fluoro-4-methoxypyrrolo[2,1-f][1,2,4]triazin-2-yl)amino)-3-fluoropiperidin-1-yl)-2-hydroxyethan-1-one [1454];
5-(1-((R)-1,1-difluoropropan-2-yl)-1H-benzo[d][1,2,3]triazol-6-yl)-6-fluoro-N-((3S,4R)-3-fluoro-1-(oxetan-3-yl)piperidin-4-yl)-4-methoxypyrrolo[2,1-f][1,2,4]triazin-2-amine [1455];
5-(1-((R)-1,1-difluoropropan-2-yl)-1H-benzo[d][1,2,3]triazol-6-yl)-6-fluoro-N-((3R,4R)-3-fluoro-1-(oxetan-3-yl)piperidin-4-yl)-4-methoxypyrrolo[2,1-f][1,2,4]triazin-2-amine [1456];
5-(1-((R)-1,1-difluoropropan-2-yl)-1H-benzo[d][1,2,3]triazol-6-yl)-6-fluoro-N-((3S,4S)-3-fluoro-1-(oxetan-3-yl)piperidin-4-yl)-4-methoxypyrrolo[2,1-f][1,2,4]triazin-2-amine [1457];
5-(1-((R)-1,1-difluoropropan-2-yl)-1H-benzo[d][1,2,3]triazol-6-yl)-6-fluoro-N-((3R,4S)-3-fluoro-1-(oxetan-3-yl)piperidin-4-yl)-4-methoxypyrrolo[2,1-f][1,2,4]triazin-2-amine [1458];
N—((R)-3,3-difluoro-1-methylpiperidin-4-yl)-5-(1-((R)-1,1-difluoropropan-2-yl)-1H-benzo[d][1,2,3]triazol-6-yl)-6-fluoro-4-methoxypyrrolo[2,1-f][1,2,4]triazin-2-amine [1459];
N—((R)-3,3-difluoro-1-(methyl-d₃)piperidin-4-yl)-5-(1-((R)-1,1-difluoropropan-2-yl)-1H-benzo[d][1,2,3]triazol-6-yl)-6-fluoro-4-methoxypyrrolo[2,1-f][1,2,4]triazin-2-amine [1460];
1-((R)-4-((5-(1-((R)-1,1-difluoropropan-2-yl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)ethan-1-one [1461];
1-((R)-4-((5-(1-((R)-1,1-difluoropropan-2-yl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)ethan-1-one-2,2,2-d₃ [1462];
1-((R)-4-((5-(1-((R)-1,1-difluoropropan-2-yl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)-2-hydroxyethan-1-one [1463];
N—((R)-3,3-difluoro-1-(methylsulfonyl)piperidin-4-yl)-5-(1-((R)-1,1-difluoropropan-2-yl)-1H-benzo[d][1,2,3]triazol-6-yl)-6-fluoro-4-methoxypyrrolo[2,1-f][1,2,4]triazin-2-amine [1464];
N—((R)-3,3-difluoro-1-(oxetan-3-yl)piperidin-4-yl)-5-(1-((R)-1,1-difluoropropan-2-yl)-1H-benzo[d][1,2,3]triazol-6-yl)-6-fluoro-4-methoxypyrrolo[2,1-f][1,2,4]triazin-2-amine [1465];
(R)-5-(1-(1,1-difluoropropan-2-yl)-1H-benzo[d][1,2,3]triazol-6-yl)-6-fluoro-4-methoxy-N-(2-oxaspiro[3.5]nonan-7-yl)pyrrolo[2,1-f][1,2,4]triazin-2-amine [1466];
3-((5-(1-(1,3-difluoropropan-2-yl)-1H-benzo[d][1,2,3]triazol-6-yl)-4-methoxypyrrolo[2,1-f][1,2,4]triazin-2-yl)amino)-2,2-dimethylpropanenitrile [1467];
N-((1s,4s)-4-((5-(1-(1,3-difluoropropan-2-yl)-1H-benzo[d][1,2,3]triazol-6-yl)-4-methoxypyrrolo[2,1-f][1,2,4]triazin-2-yl)amino)cyclohexyl)acetamide [1468];
(1s,4s)-4-((5-(1-(1,3-difluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1469];
(1r,4r)-4-((5-(1-(1,3-difluoropropan-2-yl)-1H-benzo[d][1,2,3]triazol-6-yl)-4-methoxypyrrolo[2,1-f][1,2,4]triazin-2-yl)amino)-1-methylcyclohexan-1-ol [1470];
(R)—N-(4,4-difluoro-1-methylpyrrolidin-3-yl)-5-(1-(1,3-difluoropropan-2-yl)-1H-benzo[d][1,2,3]triazol-6-yl)-4-methoxypyrrolo[2,1-f][1,2,4]triazin-2-amine [1471];
(R)-1-(4-((5-(1-(1,3-difluoropropan-2-yl)-1H-benzo[d][1,2,3]triazol-6-yl)-4-methoxypyrrolo[2,1-f][1,2,4]triazin-2-yl)amino)-3,3-difluoropyrrolidin-1-yl)ethan-1-one [1472];
(R)—N-(4,4-difluoro-1-(oxetan-3-yl)pyrrolidin-3-yl)-5-(1-(1,3-difluoropropan-2-yl)-1H-benzo[d][1,2,3]triazol-6-yl)-4-methoxypyrrolo[2,1-f][1,2,4]triazin-2-amine [1473];
5-(1-(1,3-difluoropropan-2-yl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1474];
5-(1-(1,3-difluoropropan-2-yl)-1H-benzo[d][1,2,3]triazol-6-yl)-N-((3S,4R)-3-fluoro-1-methylpiperidin-4-yl)-4-methoxypyrrolo[2,1-f][1,2,4]triazin-2-amine [1475];
5-(1-(1,3-difluoropropan-2-yl)-1H-benzo[d][1,2,3]triazol-6-yl)-N-((3R,4S)-3-fluoro-1-methylpiperidin-4-yl)-4-methoxypyrrolo[2,1-f][1,2,4]triazin-2-amine [1476];
5-(1-(1,3-difluoropropan-2-yl)-1H-benzo[d][1,2,3]triazol-6-yl)-N-((3S,4R)-3-fluoro-1-(2-methoxyethyl)piperidin-4-yl)-4-methoxypyrrolo[2,1-f][1,2,4]triazin-2-amine [1477];
5-(1-(1,3-difluoropropan-2-yl)-1H-benzo[d][1,2,3]triazol-6-yl)-N-((3R,4R)-3-fluoro-1-(2-methoxyethyl)piperidin-4-yl)-4-methoxypyrrolo[2,1-f][1,2,4]triazin-2-amine [1478];
5-(1-(1,3-difluoropropan-2-yl)-1H-benzo[d][1,2,3]triazol-6-yl)-N-((3S,4S)-3-fluoro-1-(2-methoxyethyl)piperidin-4-yl)-4-methoxypyrrolo[2,1-f][1,2,4]triazin-2-amine [1479];
5-(1-(1,3-difluoropropan-2-yl)-1H-benzo[d][1,2,3]triazol-6-yl)-N-((3R,4S)-3-fluoro-1-(2-methoxyethyl)piperidin-4-yl)-4-methoxypyrrolo[2,1-f][1,2,4]triazin-2-amine [1480];
1-((3R,4S)-4-((5-(1-(1,3-difluoropropan-2-yl)-1H-benzo[d][1,2,3]triazol-6-yl)-4-methoxypyrrolo[2,1-f][1,2,4]triazin-2-yl)amino)-3-fluoropiperidin-1-yl)ethan-1-one [1481];
1-((3R,4S)-4-((5-(1-(1,3-difluoropropan-2-yl)-1H-benzo[d][1,2,3]triazol-6-yl)-4-methoxypyrrolo[2,1-f][1,2,4]triazin-2-yl)amino)-3-fluoropiperidin-1-yl)ethan-1-one-2,2,2-d₃ [1482];
1-((3R,4S)-4-((5-(1-(1,3-difluoropropan-2-yl)-1H-benzo[d][1,2,3]triazol-6-yl)-4-methoxypyrrolo[2,1-f][1,2,4]triazin-2-yl)amino)-3-fluoropiperidin-1-yl)-2-hydroxyethan-1-one [1483];
5-(1-(1,3-difluoropropan-2-yl)-1H-benzo[d][1,2,3]triazol-6-yl)-N-((3S,4R)-3-fluoro-1-(oxetan-3-yl)piperidin-4-yl)-4-methoxypyrrolo[2,1-f][1,2,4]triazin-2-amine [1484];
5-(1-(1,3-difluoropropan-2-yl)-1H-benzo[d][1,2,3]triazol-6-yl)-N-((3R,4R)-3-fluoro-1-(oxetan-3-yl)piperidin-4-yl)-4-methoxypyrrolo[2,1-f][1,2,4]triazin-2-amine [1485];
5-(1-(1,3-difluoropropan-2-yl)-1H-benzo[d][1,2,3]triazol-6-yl)-N-((3S,4S)-3-fluoro-1-(oxetan-3-yl)piperidin-4-yl)-4-methoxypyrrolo[2,1-f][1,2,4]triazin-2-amine [1486];
5-(1-(1,3-difluoropropan-2-yl)-1H-benzo[d][1,2,3]triazol-6-yl)-N-((3R,4S)-3-fluoro-1-(oxetan-3-yl)piperidin-4-yl)-4-methoxypyrrolo[2,1-f][1,2,4]triazin-2-amine [1487];
5-(1-(1,3-difluoropropan-2-yl)-1H-benzo[d][1,2,3]triazol-6-yl)-N-((3S,4R)-3-fluoro-1-(oxetan-3-yl-3-d)piperidin-4-yl)-4-methoxypyrrolo[2,1-f][1,2,4]triazin-2-amine [1488];
5-(1-(1,3-difluoropropan-2-yl)-1H-benzo[d][1,2,3]triazol-6-yl)-N-((3R,4R)-3-fluoro-1-(oxetan-3-yl-3-d)piperidin-4-yl)-4-methoxypyrrolo[2,1-f][1,2,4]triazin-2-amine [1489];
5-(1-(1,3-difluoropropan-2-yl)-1H-benzo[d][1,2,3]triazol-6-yl)-N-((3S,4S)-3-fluoro-1-(oxetan-3-yl-3-d)piperidin-4-yl)-4-methoxypyrrolo[2,1-f][1,2,4]triazin-2-amine [1490];
5-(1-(1,3-difluoropropan-2-yl)-1H-benzo[d][1,2,3]triazol-6-yl)-N-((3R,4S)-3-fluoro-1-(oxetan-3-yl-3-d)piperidin-4-yl)-4-methoxypyrrolo[2,1-f][1,2,4]triazin-2-amine [1491];
(R)—N-(3,3-difluoro-1-methylpiperidin-4-yl)-5-(1-(1,3-difluoropropan-2-yl)-1H-benzo[d][1,2,3]triazol-6-yl)-4-methoxypyrrolo[2,1-f][1,2,4]triazin-2-amine [1492];
(R)—N-(3,3-difluoro-1-(methyl-d₃)piperidin-4-yl)-5-(1-(1,3-difluoropropan-2-yl)-1H-benzo[d][1,2,3]triazol-6-yl)-4-methoxypyrrolo[2,1-f][1,2,4]triazin-2-amine [1493];
(R)—N-(3,3-difluoro-1-(2-methoxyethyl)piperidin-4-yl)-5-(1-(1,3-difluoropropan-2-yl)-1H-benzo[d][1,2,3]triazol-6-yl)-4-methoxypyrrolo[2,1-f][1,2,4]triazin-2-amine [1494];
(R)-1-(4-((5-(1-(1,3-difluoropropan-2-yl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one [1495];
(R)-1-(4-((5-(1-(1,3-difluoropropan-2-yl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one-2,2,2-d₃ [1496];
(R)-1-(4-((5-(1-(1,3-difluoropropan-2-yl)-1H-benzo[d][1,2,3]triazol-6-yl)-4-methoxypyrrolo[2,1-f][1,2,4]triazin-2-yl)amino)-3,3-difluoropiperidin-1-yl)-2-hydroxyethan-1-one [1497];
(R)—N-(3,3-difluoro-1-(methylsulfonyl)piperidin-4-yl)-5-(1-(1,3-difluoropropan-2-yl)-1H-benzo[d][1,2,3]triazol-6-yl)-4-methoxypyrrolo[2,1-f][1,2,4]triazin-2-amine [1498];
(R)—N-(3,3-difluoro-1-(oxetan-3-yl)piperidin-4-yl)-5-(1-(1,3-difluoropropan-2-yl)-1H-benzo[d][1,2,3]triazol-6-yl)-4-methoxypyrrolo[2,1-f][1,2,4]triazin-2-amine [1499]; and
(R)—N-(3,3-difluoro-1-(oxetan-3-yl-3-d)piperidin-4-yl)-5-(1-(1,3-difluoropropan-2-yl)-1H-benzo[d][1,2,3]triazol-6-yl)-4-methoxypyrrolo[2,1-f][1,2,4]triazin-2-amine [1500]; or a pharmaceutically acceptable salt thereof.

32. The compound of claim 1, wherein the compound of Formula I is selected from the group consisting of:
5-(1-(1,3-difluoropropan-2-yl)-1H-benzo[d][1,2,3]triazol-6-yl)-4-methoxy-N-(2-oxaspiro[3.5]nonan-7-yl)pyrrolo[2,1-f][1,2,4]triazin-2-amine [1501];
3-((5-(1-(1,3-difluoropropan-2-yl)-1H-benzo[d][1,2,3]triazol-6-yl)-6-fluoro-4-methoxypyrrolo[2,1-f][1,2,4]triazin-2-yl)amino)-2,2-dimethylpropanenitrile [1502];
(1s,4s)-4-((5-(1-(1,3-difluoropropan-2-yl)-1H-benzo[d][1,2,3]triazol-6-yl)-6-fluoro-4-methoxypyrrolo[2,1-f][1,2,4]triazin-2-yl)amino)-1-methylcyclohexan-1-ol [1503];
(1r,4r)-4-((5-(1-(1,3-difluoropropan-2-yl)-1H-benzo[d][1,2,3]triazol-6-yl)-6-fluoro-4-methoxypyrrolo[2,1-f][1,2,4]triazin-2-yl)amino)-1-methylcyclohexan-1-ol [1504];
(R)—N-(4,4-difluoro-1-methylpyrrolidin-3-yl)-5-(1-(1,3-difluoropropan-2-yl)-1H-benzo[d][1,2,3]triazol-6-yl)-6-fluoro-4-methoxypyrrolo[2,1-f][1,2,4]triazin-2-amine [1505];
(R)-1-(4-((5-(1-(1,3-difluoropropan-2-yl)-1H-benzo[d][1,2,3]triazol-6-yl)-6-fluoro-4-methoxypyrrolo[2,1-f][1,2,4]triazin-2-yl)amino)-3,3-difluoropyrrolidin-1-yl)ethan-1-one [1506];
(R)—N-(4,4-difluoro-1-(oxetan-3-yl)pyrrolidin-3-yl)-5-(1-(1,3-difluoropropan-2-yl)-1H-benzo[d][1,2,3]triazol-6-yl)-6-fluoro-4-methoxypyrrolo[2,1-f][1,2,4]triazin-2-amine [1507];
5-(1-(1,3-difluoropropan-2-yl)-1H-benzo[d][1,2,3]triazol-6-yl)-6-fluoro-4-methoxy-N-(1-(oxetan-3-yl)piperidin-4-yl)pyrrolo[2,1-f][1,2,4]triazin-2-amine [1508];
5-(1-(1,3-difluoropropan-2-yl)-1H-benzo[d][1,2,3]triazol-6-yl)-6-fluoro-N-((3S,4R)-3-fluoro-1-methylpiperidin-4-yl)-4-methoxypyrrolo[2,1-f][1,2,4]triazin-2-amine [1509];
5-(1-(1,3-difluoropropan-2-yl)-1H-benzo[d][1,2,3]triazol-6-yl)-6-fluoro-N-((3R,4S)-3-fluoro-1-methylpiperidin-4-yl)-4-methoxypyrrolo[2,1-f][1,2,4]triazin-2-amine [1510];
5-(1-(1,3-difluoropropan-2-yl)-1H-benzo[d][1,2,3]triazol-6-yl)-6-fluoro-N-((3S,4R)-3-fluoro-1-(2-methoxyethyl)piperidin-4-yl)-4-methoxypyrrolo[2,1-f][1,2,4]triazin-2-amine [1511];
5-(1-(1,3-difluoropropan-2-yl)-1H-benzo[d][1,2,3]triazol-6-yl)-6-fluoro-N-((3R,4R)-3-fluoro-1-(2-methoxyethyl)piperidin-4-yl)-4-methoxypyrrolo[2,1-f][1,2,4]triazin-2-amine [1512];
5-(1-(1,3-difluoropropan-2-yl)-1H-benzo[d][1,2,3]triazol-6-yl)-6-fluoro-N-((3S,4S)-3-fluoro-1-(2-methoxyethyl)piperidin-4-yl)-4-methoxypyrrolo[2,1-f][1,2,4]triazin-2-amine [1513];
5-(1-(1,3-difluoropropan-2-yl)-1H-benzo[d][1,2,3]triazol-6-yl)-6-fluoro-N-((3R,4S)-3-fluoro-1-(2-methoxyethyl)piperidin-4-yl)-4-methoxypyrrolo[2,1-f][1,2,4]triazin-2-amine [1514];
1-((3R,4S)-4-((5-(1-(1,3-difluoropropan-2-yl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one [1515];
1-((3R,4S)-4-((5-(1-(1,3-difluoropropan-2-yl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one-2,2,2-d₃ [1516];
1-((3R,4S)-4-((5-(1-(1,3-difluoropropan-2-yl)-1H-benzo[d][1,2,3]triazol-6-yl)-6-fluoro-4-methoxypyrrolo[2,1-f][1,2,4]triazin-2-yl)amino)-3-fluoropiperidin-1-yl)-2-hydroxyethan-1-one [1517];
5-(1-(1,3-difluoropropan-2-yl)-1H-benzo[d][1,2,3]triazol-6-yl)-6-fluoro-N-((3S,4R)-3-fluoro-1-(oxetan-3-yl)piperidin-4-yl)-4-methoxypyrrolo[2,1-f][1,2,4]triazin-2-amine [1518];
5-(1-(1,3-difluoropropan-2-yl)-1H-benzo[d][1,2,3]triazol-6-yl)-6-fluoro-N-((3R,4R)-3-fluoro-1-(oxetan-3-yl)piperidin-4-yl)-4-methoxypyrrolo[2,1-f][1,2,4]triazin-2-amine [1519];
5-(1-(1,3-difluoropropan-2-yl)-1H-benzo[d][1,2,3]triazol-6-yl)-6-fluoro-N-((3S,4S)-3-fluoro-1-(oxetan-3-yl)piperidin-4-yl)-4-methoxypyrrolo[2,1-f][1,2,4]triazin-2-amine [1520];
5-(1-(1,3-difluoropropan-2-yl)-1H-benzo[d][1,2,3]triazol-6-yl)-6-fluoro-N-((3R,4S)-3-fluoro-1-(oxetan-3-yl)piperidin-4-yl)-4-methoxypyrrolo[2,1-f][1,2,4]triazin-2-amine [1521];
5-(1-(1,3-difluoropropan-2-yl)-1H-benzo[d][1,2,3]triazol-6-yl)-6-fluoro-N-((3S,4R)-3-fluoro-1-(oxetan-3-yl-3-d)piperidin-4-yl)-4-methoxypyrrolo[2,1-f][1,2,4]triazin-2-amine [1522];
5-(1-(1,3-difluoropropan-2-yl)-1H-benzo[d][1,2,3]triazol-6-yl)-6-fluoro-N-((3R,4R)-3-fluoro-1-(oxetan-3-yl-3-d)piperidin-4-yl)-4-methoxypyrrolo[2,1-f][1,2,4]triazin-2-amine [1523];
5-(1-(1,3-difluoropropan-2-yl)-1H-benzo[d][1,2,3]triazol-6-yl)-6-fluoro-N-((3S,4S)-3-fluoro-1-(oxetan-3-yl-3-d)piperidin-4-yl)-4-methoxypyrrolo[2,1-f][1,2,4]triazin-2-amine [1524];
5-(1-(1,3-difluoropropan-2-yl)-1H-benzo[d][1,2,3]triazol-6-yl)-6-fluoro-N-((3R,4S)-3-fluoro-1-(oxetan-3-yl-3-d)piperidin-4-yl)-4-methoxypyrrolo[2,1-f][1,2,4]triazin-2-amine [1525];
(R)—N-(3,3-difluoro-1-methylpiperidin-4-yl)-5-(1-(1,3-difluoropropan-2-yl)-1H-benzo[d][1,2,3]triazol-6-yl)-6-fluoro-4-methoxypyrrolo[2,1-f][1,2,4]triazin-2-amine [1526];
(R)—N-(3,3-difluoro-1-(methyl-d₃)piperidin-4-yl)-5-(1-(1,3-difluoropropan-2-yl)-1H-benzo[d][1,2,3]triazol-6-yl)-6-fluoro-4-methoxypyrrolo[2,1-f][1,2,4]triazin-2-amine [1527];
(R)—N-(3,3-difluoro-1-(2-methoxyethyl)piperidin-4-yl)-5-(1-(1,3-difluoropropan-2-yl)-1H-benzo[d][1,2,3]triazol-6-yl)-6-fluoro-4-methoxypyrrolo[2,1-f][1,2,4]triazin-2-amine [1528];
(R)-1-(4-((5-(1-(1,3-difluoropropan-2-yl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)ethan-1-one [1529];
(R)-1-(4-((5-(1-(1,3-difluoropropan-2-yl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)ethan-1-one-2,2,2-d₃ [1530];
(R)-1-(4-((5-(1-(1,3-difluoropropan-2-yl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)-2-hydroxyethan-1-one [1531];
(R)—N-(3,3-difluoro-1-(methylsulfonyl)piperidin-4-yl)-5-(1-(1,3-difluoropropan-2-yl)-1H-benzo[d][1,2,3]triazol-6-yl)-6-fluoro-4-methoxypyrrolo[2,1-f][1,2,4]triazin-2-amine [1532];
(R)—N-(3,3-difluoro-1-(oxetan-3-yl)piperidin-4-yl)-5-(1-(1,3-difluoropropan-2-yl)-1H-benzo[d][1,2,3]triazol-6-yl)-6-fluoro-4-methoxypyrrolo[2,1-f][1,2,4]triazin-2-amine [1533];
(R)—N-(3,3-difluoro-1-(oxetan-3-yl-3-d)piperidin-4-yl)-5-(1-(1,3-difluoropropan-2-yl)-1H-benzo[d][1,2,3]triazol-6-yl)-6-fluoro-4-methoxypyrrolo[2,1-f][1,2,4]triazin-2-amine [1534];
5-(1-(1,3-difluoropropan-2-yl)-1H-benzo[d][1,2,3]triazol-6-yl)-6-fluoro-4-methoxy-N-(2-oxaspiro[3.5]nonan-7-yl)pyrrolo[2,1-f][1,2,4]triazin-2-amine [1535];
(S)-3-((4-methoxy-5-(1-(1,1,1-trifluoropropan-2-yl)-1H-benzo[d][1,2,3]triazol-6-yl)pyrrolo[2,1-f][1,2,4]triazin-2-yl)amino)-2,2-dimethylpropanenitrile [1536];
(1R,4s)-4-((4-methoxy-5-(1-((S)-1,1,1-trifluoropropan-2-yl)-1H-benzo[d][1,2,3]triazol-6-yl)pyrrolo[2,1-f][1,2,4]triazin-2-yl)amino)-1-methylcyclohexan-1-ol [1537];
(1S,4r)-4-((4-methoxy-5-(1-((S)-1,1,1-trifluoropropan-2-yl)-1H-benzo[d][1,2,3]triazol-6-yl)pyrrolo[2,1-f][1,2,4]triazin-2-yl)amino)-1-methylcyclohexan-1-ol [1538];
N—((R)-4,4-difluoro-1-methylpyrrolidin-3-yl)-4-methoxy-5-(1-((S)-1,1,1-trifluoropropan-2-yl)-1H-benzo[d][1,2,3]triazol-6-yl)pyrrolo[2,1-f][1,2,4]triazin-2-amine [1539];
1-((R)-3,3-difluoro-4-((4-methoxy-5-(1-((S)-1,1,1-trifluoropropan-2-yl)-1H-benzo[d][1,2,3]triazol-6-yl)pyrrolo[2,1-f][1,2,4]triazin-2-yl)amino)pyrrolidin-1-yl)ethan-1-one [1540];
N—((R)-4,4-difluoro-1-(oxetan-3-yl)pyrrolidin-3-yl)-4-methoxy-5-(1-((S)-1,1,1-trifluoropropan-2-yl)-1H-benzo[d][1,2,3]triazol-6-yl)pyrrolo[2,1-f][1,2,4]triazin-2-amine [1541];
(S)-4-methoxy-N-(1-(oxetan-3-yl)piperidin-4-yl)-5-(1-(1,1,1-trifluoropropan-2-yl)-1H-benzo[d][1,2,3]triazol-6-yl)pyrrolo[2,1-f][1,2,4]triazin-2-amine [1542];
N-((3S,4R)-3-fluoro-1-methylpiperidin-4-yl)-4-methoxy-5-(1-((S)-1,1,1-trifluoropropan-2-yl)-1H-benzo[d][1,2,3]triazol-6-yl)pyrrolo[2,1-f][1,2,4]triazin-2-amine [1543];
N-((3R,4S)-3-fluoro-1-methylpiperidin-4-yl)-4-methoxy-5-(1-((S)-1,1,1-trifluoropropan-2-yl)-1H-benzo[d][1,2,3]triazol-6-yl)pyrrolo[2,1-f][1,2,4]triazin-2-amine [1544];
1-((3R,4S)-3-fluoro-4-((4-methoxy-5-(1-((S)-1,1,1-trifluoropropan-2-yl)-1H-benzo[d][1,2,3]triazol-6-yl)pyrrolo[2,1-f][1,2,4]triazin-2-yl)amino)piperidin-1-yl)ethan-1-one [1545];
1-((3R,4S)-3-fluoro-4-((4-methoxy-5-(1-((S)-1,1,1-trifluoropropan-2-yl)-1H-benzo[d][1,2,3]triazol-6-yl)pyrrolo[2,1-f][1,2,4]triazin-2-yl)amino)piperidin-1-yl)ethan-1-one-2,2,2-d₃ [1546];
1-((3R,4S)-3-fluoro-4-((4-methoxy-5-(1-((S)-1,1,1-trifluoropropan-2-yl)-1H-benzo[d][1,2,3]triazol-6-yl)pyrrolo[2,1-f][1,2,4]triazin-2-yl)amino)piperidin-1-yl)-2-hydroxyethan-1-one [1547];
N-((3S,4R)-3-fluoro-1-(oxetan-3-yl)piperidin-4-yl)-4-methoxy-5-(1-((S)-1,1,1-trifluoropropan-2-yl)-1H-benzo[d][1,2,3]triazol-6-yl)pyrrolo[2,1-f][1,2,4]triazin-2-amine [1548];
N-((3R,4R)-3-fluoro-1-(oxetan-3-yl)piperidin-4-yl)-4-methoxy-5-(1-((S)-1,1,1-trifluoropropan-2-yl)-1H-benzo[d][1,2,3]triazol-6-yl)pyrrolo[2,1-f][1,2,4]triazin-2-amine [1549];
N-((3S,4S)-3-fluoro-1-(oxetan-3-yl)piperidin-4-yl)-4-methoxy-5-(1-((S)-1,1,1-trifluoropropan-2-yl)-1H-benzo[d][1,2,3]triazol-6-yl)pyrrolo[2,1-f][1,2,4]triazin-2-amine [1550];
N-((3R,4S)-3-fluoro-1-(oxetan-3-yl)piperidin-4-yl)-4-methoxy-5-(1-((S)-1,1,1-trifluoropropan-2-yl)-1H-benzo[d][1,2,3]triazol-6-yl)pyrrolo[2,1-f][1,2,4]triazin-2-amine [1551];
N—((R)-3,3-difluoro-1-methylpiperidin-4-yl)-4-methoxy-5-(1-((S)-1,1,1-trifluoropropan-2-yl)-1H-benzo[d][1,2,3]triazol-6-yl)pyrrolo[2,1-f][1,2,4]triazin-2-amine [1552];
N—((R)-3,3-difluoro-1-(methyl-d₃)piperidin-4-yl)-4-methoxy-5-(1-((S)-1,1,1-trifluoropropan-2-yl)-1H-benzo[d][1,2,3]triazol-6-yl)pyrrolo[2,1-f][1,2,4]triazin-2-amine [1553];
1-((R)-3,3-difluoro-4-((4-methoxy-5-(1-((S)-1,1,1-trifluoropropan-2-yl)-1H-benzo[d][1,2,3]triazol-6-yl)pyrrolo[2,1-f][1,2,4]triazin-2-yl)amino)piperidin-1-yl)ethan-1-one [1554];
1-((R)-3,3-difluoro-4-((4-methoxy-5-(1-((S)-1,1,1-trifluoropropan-2-yl)-1H-benzo[d][1,2,3]triazol-6-yl)pyrrolo[2,1-f][1,2,4]triazin-2-yl)amino)piperidin-1-yl)ethan-1-one-2,2,2-d₃ [1555];
1-((R)-3,3-difluoro-4-((4-methoxy-5-(1-((S)-1,1,1-trifluoropropan-2-yl)-1H-benzo[d][1,2,3]triazol-6-yl)pyrrolo[2,1-f][1,2,4]triazin-2-yl)amino)piperidin-1-yl)-2-hydroxyethan-1-one [1556];
N—((R)-3,3-difluoro-1-(methylsulfonyl)piperidin-4-yl)-4-methoxy-5-(1-((S)-1,1,1-trifluoropropan-2-yl)-1H-benzo[d][1,2,3]triazol-6-yl)pyrrolo[2,1-f][1,2,4]triazin-2-amine [1557];
N—((R)-3,3-difluoro-1-(oxetan-3-yl)piperidin-4-yl)-4-methoxy-5-(1-((S)-1,1,1-trifluoropropan-2-yl)-1H-benzo[d][1,2,3]triazol-6-yl)pyrrolo[2,1-f][1,2,4]triazin-2-amine [1558];
(S)-4-methoxy-N-(2-oxaspiro[3.5]nonan-7-yl)-5-(1-(1,1,1-trifluoropropan-2-yl)-1H-benzo[d][1,2,3]triazol-6-yl)pyrrolo[2,1-f][1,2,4]triazin-2-amine [1559];
(R)-3-((4-methoxy-5-(1-(1,1,1-trifluoropropan-2-yl)-1H-benzo[d][1,2,3]triazol-6-yl)pyrrolo[2,1-f][1,2,4]triazin-2-yl)amino)-2,2-dimethylpropanenitrile [1560];
(1S,4s)-4-((4-methoxy-5-(1-((R)-1,1,1-trifluoropropan-2-yl)-1H-benzo[d][1,2,3]triazol-6-yl)pyrrolo[2,1-f][1,2,4]triazin-2-yl)amino)-1-methylcyclohexan-1-ol [1561];
(1R,4r)-4-((4-methoxy-5-(1-((R)-1,1,1-trifluoropropan-2-yl)-1H-benzo[d][1,2,3]triazol-6-yl)pyrrolo[2,1-f][1,2,4]triazin-2-yl)amino)-1-methylcyclohexan-1-ol [1562];
N—((R)-4,4-difluoro-1-methylpyrrolidin-3-yl)-4-methoxy-5-(1-((R)-1,1,1-trifluoropropan-2-yl)-1H-benzo[d][1,2,3]triazol-6-yl)pyrrolo[2,1-f][1,2,4]triazin-2-amine [1563];
1-((R)-3,3-difluoro-4-((4-methoxy-5-(1-((R)-1,1,1-trifluoropropan-2-yl)-1H-benzo[d][1,2,3]triazol-6-yl)pyrrolo[2,1-f][1,2,4]triazin-2-yl)amino)pyrrolidin-1-yl)ethan-1-one [1564];
N—((R)-4,4-difluoro-1-(oxetan-3-yl)pyrrolidin-3-yl)-4-methoxy-5-(1-((R)-1,1,1-trifluoropropan-2-yl)-1H-benzo[d][1,2,3]triazol-6-yl)pyrrolo[2,1-f][1,2,4]triazin-2-amine [1565];
(R)-4-methoxy-N-(1-(oxetan-3-yl)piperidin-4-yl)-5-(1-(1,1,1-trifluoropropan-2-yl)-1H-benzo[d][1,2,3]triazol-6-yl)pyrrolo[2,1-f][1,2,4]triazin-2-amine [1566];
N-((3S,4R)-3-fluoro-1-methylpiperidin-4-yl)-4-methoxy-5-(1-((R)-1,1,1-trifluoropropan-2-yl)-1H-benzo[d][1,2,3]triazol-6-yl)pyrrolo[2,1-f][1,2,4]triazin-2-amine [1567];
N-((3R,4S)-3-fluoro-1-methylpiperidin-4-yl)-4-methoxy-5-(1-((R)-1,1,1-trifluoropropan-2-yl)-1H-benzo[d][1,2,3]triazol-6-yl)pyrrolo[2,1-f][1,2,4]triazin-2-amine [1568];
1-((3R,4S)-3-fluoro-4-((4-methoxy-5-(1-((R)-1,1,1-trifluoropropan-2-yl)-1H-benzo[d][1,2,3]triazol-6-yl)pyrrolo[2,1-f][1,2,4]triazin-2-yl)amino)piperidin-1-yl)ethan-1-one [1569];
1-((3R,4S)-3-fluoro-4-((4-methoxy-5-(1-((R)-1,1,1-trifluoropropan-2-yl)-1H-benzo[d][1,2,3]triazol-6-yl)pyrrolo[2,1-f][1,2,4]triazin-2-yl)amino)piperidin-1-yl)ethan-1-one-2,2,2-d₃ [1570];
1-((3R,4S)-3-fluoro-4-((4-methoxy-5-(1-((R)-1,1,1-trifluoropropan-2-yl)-1H-benzo[d][1,2,3]triazol-6-yl)pyrrolo[2,1-f][1,2,4]triazin-2-yl)amino)piperidin-1-yl)-2-hydroxyethan-1-one [1571];
N-((3S,4R)-3-fluoro-1-(oxetan-3-yl)piperidin-4-yl)-4-methoxy-5-(1-((R)-1,1,1-trifluoropropan-2-yl)-1H-benzo[d][1,2,3]triazol-6-yl)pyrrolo[2,1-f][1,2,4]triazin-2-amine [1572];
N-((3R,4R)-3-fluoro-1-(oxetan-3-yl)piperidin-4-yl)-4-methoxy-5-(1-((R)-1,1,1-trifluoropropan-2-yl)-1H-benzo[d][1,2,3]triazol-6-yl)pyrrolo[2,1-f][1,2,4]triazin-2-amine [1573];
N-((3S,4S)-3-fluoro-1-(oxetan-3-yl)piperidin-4-yl)-4-methoxy-5-(1-((R)-1,1,1-trifluoropropan-2-yl)-1H-benzo[d][1,2,3]triazol-6-yl)pyrrolo[2,1-f][1,2,4]triazin-2-amine [1574];
N-((3R,4S)-3-fluoro-1-(oxetan-3-yl)piperidin-4-yl)-4-methoxy-5-(1-((R)-1,1,1-trifluoropropan-2-yl)-1H-benzo[d][1,2,3]triazol-6-yl)pyrrolo[2,1-f][1,2,4]triazin-2-amine [1575];
N—((R)-3,3-difluoro-1-methylpiperidin-4-yl)-4-methoxy-5-(1-((R)-1,1,1-trifluoropropan-2-yl)-1H-benzo[d][1,2,3]triazol-6-yl)pyrrolo[2,1-f][1,2,4]triazin-2-amine [1576];
N—((R)-3,3-difluoro-1-(methyl-d₃)piperidin-4-yl)-4-methoxy-5-(1-((R)-1,1,1-trifluoropropan-2-yl)-1H-benzo[d][1,2,3]triazol-6-yl)pyrrolo[2,1-f][1,2,4]triazin-2-amine [1577];
1-((R)-3,3-difluoro-4-((4-methoxy-5-(1-((R)-1,1,1-trifluoropropan-2-yl)-1H-benzo[d][1,2,3]triazol-6-yl)pyrrolo[2,1-f][1,2,4]triazin-2-yl)amino)piperidin-1-yl)ethan-1-one [1578];
1-((R)-3,3-difluoro-4-((4-methoxy-5-(1-((R)-1,1,1-trifluoropropan-2-yl)-1H-benzo[d][1,2,3]triazol-6-yl)pyrrolo[2,1-f][1,2,4]triazin-2-yl)amino)piperidin-1-yl)ethan-1-one-2,2,2-d₃ [1579];
1-((R)-3,3-difluoro-4-((4-methoxy-5-(1-((R)-1,1,1-trifluoropropan-2-yl)-1H-benzo[d][1,2,3]triazol-6-yl)pyrrolo[2,1-f][1,2,4]triazin-2-yl)amino)piperidin-1-yl)-2-hydroxyethan-1-one [1580];
N—((R)-3,3-difluoro-1-(methylsulfonyl)piperidin-4-yl)-4-methoxy-5-(1-((R)-1,1,1-trifluoropropan-2-yl)-1H-benzo[d][1,2,3]triazol-6-yl)pyrrolo[2,1-f][1,2,4]triazin-2-amine [1581];
N—((R)-3,3-difluoro-1-(oxetan-3-yl)piperidin-4-yl)-4-methoxy-5-(1-((R)-1,1,1-trifluoropropan-2-yl)-1H-benzo[d][1,2,3]triazol-6-yl)pyrrolo[2,1-f][1,2,4]triazin-2-amine [1582];
(R)-4-methoxy-N-(2-oxaspiro[3.5]nonan-7-yl)-5-(1-(1,1,1-trifluoropropan-2-yl)-1H-benzo[d][1,2,3]triazol-6-yl)pyrrolo[2,1-f][1,2,4]triazin-2-amine [1583];
3-((5-(1-(3,3-difluorocyclobutyl)-1H-benzo[d][1,2,3]triazol-6-yl)-4-methoxypyrrolo[2,1-f][1,2,4]triazin-2-yl)amino)-2,2-dimethylpropanenitrile [1584];
(1s,4s)-4-((5-(1-(3,3-difluorocyclobutyl)-1H-benzo[d][1,2,3]triazol-6-yl)-4-methoxypyrrolo[2,1-f][1,2,4]triazin-2-yl)amino)-1-methylcyclohexan-1-ol [1585];
(1r,4r)-4-((5-(1-(3,3-difluorocyclobutyl)-1H-benzo[d][1,2,3]triazol-6-yl)-4-methoxypyrrolo[2,1-f][1,2,4]triazin-2-yl)amino)-1-methylcyclohexan-1-ol [1586];
(R)—N-(4,4-difluoro-1-methylpyrrolidin-3-yl)-5-(1-(3,3-difluorocyclobutyl)-1H-benzo[d][1,2,3]triazol-6-yl)-4-methoxypyrrolo[2,1-f][1,2,4]triazin-2-amine [1587];
(R)-1-(4-((5-(1-(3,3-difluorocyclobutyl)-1H-benzo[d][1,2,3]triazol-6-yl)-4-methoxypyrrolo[2,1-f][1,2,4]triazin-2-yl)amino)-3,3-difluoropyrrolidin-1-yl)ethan-1-one [1588];
(R)—N-(4,4-difluoro-1-(oxetan-3-yl)pyrrolidin-3-yl)-5-(1-(3,3-difluorocyclobutyl)-1H-benzo[d][1,2,3]triazol-6-yl)-4-methoxypyrrolo[2,1-f][1,2,4]triazin-2-amine [1589];
5-(1-(3,3-difluorocyclobutyl)-1H-benzo[d][1,2,3]triazol-6-yl)-4-methoxy-N-(1-(oxetan-3-yl)piperidin-4-yl)pyrrolo[2,1-f][1,2,4]triazin-2-amine [1590];
5-(1-(3,3-difluorocyclobutyl)-1H-benzo[d][1,2,3]triazol-6-yl)-N-((3S,4R)-3-fluoro-1-methylpiperidin-4-yl)-4-methoxypyrrolo[2,1-f][1,2,4]triazin-2-amine [1591];
5-(1-(3,3-difluorocyclobutyl)-1H-benzo[d][1,2,3]triazol-6-yl)-N-((3R,4S)-3-fluoro-1-methylpiperidin-4-yl)-4-methoxypyrrolo[2,1-f][1,2,4]triazin-2-amine [1592];
1-((3R,4S)-4-((5-(1-(3,3-difluorocyclobutyl)-1H-benzo[d][1,2,3]triazol-6-yl)-4-methoxypyrrolo[2,1-f][1,2,4]triazin-2-yl)amino)-3-fluoropiperidin-1-yl)ethan-1-one [1593];
1-((3R,4S)-4-((5-(1-(3,3-difluorocyclobutyl)-1H-benzo[d][1,2,3]triazol-6-yl)-4-methoxypyrrolo[2,1-f][1,2,4]triazin-2-yl)amino)-3-fluoropiperidin-1-yl)ethan-1-one-2,2,2-d₃ [1594];
1-((3R,4S)-4-((5-(1-(3,3-difluorocyclobutyl)-1H-benzo[d][1,2,3]triazol-6-yl)-4-methoxypyrrolo[2,1-f][1,2,4]triazin-2-yl)amino)-3-fluoropiperidin-1-yl)-2-hydroxyethan-1-one [1595];
5-(1-(3,3-difluorocyclobutyl)-1H-benzo[d][1,2,3]triazol-6-yl)-N-((3S,4R)-3-fluoro-1-(oxetan-3-yl)piperidin-4-yl)-4-methoxypyrrolo[2,1-f][1,2,4]triazin-2-amine [1596];
5-(1-(3,3-difluorocyclobutyl)-1H-benzo[d][1,2,3]triazol-6-yl)-N-((3R,4R)-3-fluoro-1-(oxetan-3-yl)piperidin-4-yl)-4-methoxypyrrolo[2,1-f][1,2,4]triazin-2-amine [1597];
5-(1-(3,3-difluorocyclobutyl)-1H-benzo[d][1,2,3]triazol-6-yl)-N-((3S,4S)-3-fluoro-1-(oxetan-3-yl)piperidin-4-yl)-4-methoxypyrrolo[2,1-f][1,2,4]triazin-2-amine [1598];
5-(1-(3,3-difluorocyclobutyl)-1H-benzo[d][1,2,3]triazol-6-yl)-N-((3R,4S)-3-fluoro-1-(oxetan-3-yl)piperidin-4-yl)-4-methoxypyrrolo[2,1-f][1,2,4]triazin-2-amine [1599]; and
(R)—N-(3,3-difluoro-1-methylpiperidin-4-yl)-5-(1-(3,3-difluorocyclobutyl)-1H-benzo[d][1,2,3]triazol-6-yl)-4-methoxypyrrolo[2,1-f][1,2,4]triazin-2-amine [1600]; or a pharmaceutically acceptable salt thereof.

33. The compound of claim 1, wherein the compound of Formula I is selected from the group consisting of
(R)—N-(3,3-difluoro-1-(methyl-d₃)piperidin-4-yl)-5-(1-(3,3-difluorocyclobutyl)-1H-benzo[d][1,2,3]triazol-6-yl)-4-methoxypyrrolo[2,1-f][1,2,4]triazin-2-amine [1601];
(R)-1-(4-((5-(1-(3,3-difluorocyclobutyl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one [1602];
(R)-1-(4-((5-(1-(3,3-difluorocyclobutyl)-1H-benzo[d][1,2,3]triazol-6-yl)-4-methoxypyrrolo[2,1-f][1,2,4]triazin-2-yl)amino)-3,3-difluoropiperidin-1-yl)ethan-1-one-2,2,2-d₃ [1603];
(R)-1-(4-((5-(1-(3,3-difluorocyclobutyl)-1H-benzo[d][1,2,3]triazol-6-yl)-4-methoxypyrrolo[2,1-f][1,2,4]triazin-2-yl)amino)-3,3-difluoropiperidin-1-yl)-2-hydroxyethan-1-one [1604];
(R)—N-(3,3-difluoro-1-(methylsulfonyl)piperidin-4-yl)-5-(1-(3,3-difluorocyclobutyl)-1H-benzo[d][1,2,3]triazol-6-yl)-4-methoxypyrrolo[2,1-f][1,2,4]triazin-2-amine [1605];
(R)—N-(3,3-difluoro-1-(oxetan-3-yl)piperidin-4-yl)-5-(1-(3,3-difluorocyclobutyl)-1H-benzo[d][1,2,3]triazol-6-yl)-4-methoxypyrrolo[2,1-f][1,2,4]triazin-2-amine [1606];
5-(1-(3,3-difluorocyclobutyl)-1H-benzo[d][1,2,3]triazol-6-yl)-4-methoxy-N-(2-oxaspiro[3.5]nonan-7-yl)pyrrolo[2,1-f][1,2,4]triazin-2-amine [1607];
3-((5-(1-(3,3-difluorocyclobutyl)-1H-benzo[d][1,2,3]triazol-6-yl)-6-fluoro-4-methoxypyrrolo[2,1-f][1,2,4]triazin-2-yl)amino)-2,2-dimethylpropanenitrile [1608];
(1s,4s)-4-((5-(1-(3,3-difluorocyclobutyl)-1H-benzo[d][1,2,3]triazol-6-yl)-6-fluoro-4-methoxypyrrolo[2,1-f][1,2,4]triazin-2-yl)amino)-1-methylcyclohexan-1-ol [1609];
(1r,4r)-4-((5-(1-(3,3-difluorocyclobutyl)-1H-benzo[d][1,2,3]triazol-6-yl)-6-fluoro-4-methoxypyrrolo[2,1-f][1,2,4]triazin-2-yl)amino)-1-methylcyclohexan-1-ol [1610];
(R)—N-(4,4-difluoro-1-methylpyrrolidin-3-yl)-5-(1-(3,3-difluorocyclobutyl)-1H-benzo[d][1,2,3]triazol-6-yl)-6-fluoro-4-methoxypyrrolo[2,1-f][1,2,4]triazin-2-amine [1611];
(R)-1-(4-((5-(1-(3,3-difluorocyclobutyl)-1H-benzo[d][1,2,3]triazol-6-yl)-6-fluoro-4-methoxypyrrolo[2,1-f][1,2,4]triazin-2-yl)amino)-3,3-difluoropyrrolidin-1-yl)ethan-1-one [1612];
(R)—N-(4,4-difluoro-1-(oxetan-3-yl)pyrrolidin-3-yl)-5-(1-(3,3-difluorocyclobutyl)-1H-benzo[d][1,2,3]triazol-6-yl)-6-fluoro-4-methoxypyrrolo[2,1-f][1,2,4]triazin-2-amine [1613];
5-(1-(3,3-difluorocyclobutyl)-1H-benzo[d][1,2,3]triazol-6-yl)-6-fluoro-4-methoxy-N-(1-(oxetan-3-yl)piperidin-4-yl)pyrrolo[2,1-f][1,2,4]triazin-2-amine [1614];
5-(1-(3,3-difluorocyclobutyl)-1H-benzo[d][1,2,3]triazol-6-yl)-6-fluoro-N-((3S,4R)-3-fluoro-1-methylpiperidin-4-yl)-4-methoxypyrrolo[2,1-f][1,2,4]triazin-2-amine [1615];
5-(1-(3,3-difluorocyclobutyl)-1H-benzo[d][1,2,3]triazol-6-yl)-6-fluoro-N-((3R,4S)-3-fluoro-1-methylpiperidin-4-yl)-4-methoxypyrrolo[2,1-f][1,2,4]triazin-2-amine [1616];
1-((3R,4S)-4-((5-(1-(3,3-difluorocyclobutyl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one [1617];
1-((3R,4S)-4-((5-(1-(3,3-difluorocyclobutyl)-1H-benzo[d][1,2,3]triazol-6-yl)-6-fluoro-4-methoxypyrrolo[2,1-f][1,2,4]triazin-2-yl)amino)-3-fluoropiperidin-1-yl)ethan-1-one-2,2,2-d₃ [1618];
1-((3R,4S)-4-((5-(1-(3,3-difluorocyclobutyl)-1H-benzo[d][1,2,3]triazol-6-yl)-6-fluoro-4-methoxypyrrolo[2,1-f][1,2,4]triazin-2-yl)amino)-3-fluoropiperidin-1-yl)-2-hydroxyethan-1-one [1619];
5-(1-(3,3-difluorocyclobutyl)-1H-benzo[d][1,2,3]triazol-6-yl)-6-fluoro-N-((3S,4R)-3-fluoro-1-(oxetan-3-yl)piperidin-4-yl)-4-methoxypyrrolo[2,1-f][1,2,4]triazin-2-amine [1620];
5-(1-(3,3-difluorocyclobutyl)-1H-benzo[d][1,2,3]triazol-6-yl)-6-fluoro-N-((3R,4R)-3-fluoro-1-(oxetan-3-yl)piperidin-4-yl)-4-methoxypyrrolo[2,1-f][1,2,4]triazin-2-amine [1621];
5-(1-(3,3-difluorocyclobutyl)-1H-benzo[d][1,2,3]triazol-6-yl)-6-fluoro-N-((3S,4S)-3-fluoro-1-(oxetan-3-yl)piperidin-4-yl)-4-methoxypyrrolo[2,1-f][1,2,4]triazin-2-amine [1622];
5-(1-(3,3-difluorocyclobutyl)-1H-benzo[d][1,2,3]triazol-6-yl)-6-fluoro-N-((3R,4S)-3-fluoro-1-(oxetan-3-yl)piperidin-4-yl)-4-methoxypyrrolo[2,1-f][1,2,4]triazin-2-amine [1623];
(R)—N-(3,3-difluoro-1-methylpiperidin-4-yl)-5-(1-(3,3-difluorocyclobutyl)-1H-benzo[d][1,2,3]triazol-6-yl)-6-fluoro-4-methoxypyrrolo[2,1-f][1,2,4]triazin-2-amine [1624];
(R)—N-(3,3-difluoro-1-(methyl-d₃)piperidin-4-yl)-5-(1-(3,3-difluorocyclobutyl)-1H-benzo[d][1,2,3]triazol-6-yl)-6-fluoro-4-methoxypyrrolo[2,1-f][1,2,4]triazin-2-amine [1625];
(R)-1-(4-((5-(1-(3,3-difluorocyclobutyl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)ethan-1-one [1626];
(R)-1-(4-((5-(1-(3,3-difluorocyclobutyl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)ethan-1-one-2,2,2-d₃ [1627];
(R)-1-(4-((5-(1-(3,3-difluorocyclobutyl)-1H-benzo[d][1,2,3]triazol-6-yl)-6-fluoro-4-methoxypyrrolo[2,1-f][1,2,4]triazin-2-yl)amino)-3,3-difluoropiperidin-1-yl)-2-hydroxyethan-1-one [1628];
(R)—N-(3,3-difluoro-1-(methylsulfonyl)piperidin-4-yl)-5-(1-(3,3-difluorocyclobutyl)-1H-benzo[d][1,2,3]triazol-6-yl)-6-fluoro-4-methoxypyrrolo[2,1-f][1,2,4]triazin-2-amine [1629];
(R)—N-(3,3-difluoro-1-(oxetan-3-yl)piperidin-4-yl)-5-(1-(3,3-difluorocyclobutyl)-1H-benzo[d][1,2,3]triazol-6-yl)-6-fluoro-4-methoxypyrrolo[2,1-f][1,2,4]triazin-2-amine [1630];
5-(1-(3,3-difluorocyclobutyl)-1H-benzo[d][1,2,3]triazol-6-yl)-6-fluoro-4-methoxy-N-(2-oxaspiro[3.5]nonan-7-yl)pyrrolo[2,1-f][1,2,4]triazin-2-amine [1631];
N-(3,3-dimethylcyclobutyl)-5-(imidazo[1,2-a]pyrimidin-6-yl)-4-methoxypyrrolo[2,1-f][1,2,4]triazin-2-amine [1632];
N-((1s,3s)-3-((5-(imidazo[1,2-a]pyrimidin-6-yl)-4-methoxypyrrolo[2,1-f][1,2,4]triazin-2-yl)amino)-1-methylcyclobutyl)cyclopropanecarboxamide [1633];
N-((1r,3r)-3-((5-(imidazo[1,2-a]pyrimidin-6-yl)-4-methoxypyrrolo[2,1-f][1,2,4]triazin-2-yl)amino)-1-methylcyclobutyl)cyclopropanecarboxamide [1634];
1-fluoro-N-((1s,3s)-3-((5-(imidazo[1,2-a]pyrimidin-6-yl)-4-methoxypyrrolo[2,1-f][1,2,4]triazin-2-yl)amino)-1-methylcyclobutyl)cyclopropane-1-carboxamide [1635];
1-fluoro-N-((1r,3r)-3-((5-(imidazo[1,2-a]pyrimidin-6-yl)-4-methoxypyrrolo[2,1-f][1,2,4]triazin-2-yl)amino)-1-methylcyclobutyl)cyclopropane-1-carboxamide [1636];
5-(imidazo[1,2-a]pyrimidin-6-yl)-4-methoxy-N-(spiro[2.3]hexan-5-yl)pyrrolo[2,1-f][1,2,4]triazin-2-amine [1637];
5-(imidazo[1,2-a]pyrimidin-6-yl)-4-methoxy-N-(spiro[3.3]heptan-2-yl)pyrrolo[2,1-f][1,2,4]triazin-2-amine [1638];
N-(6,6-difluorospiro[3.3]heptan-2-yl)-5-(imidazo[1,2-a]pyrimidin-6-yl)-4-methoxypyrrolo[2,1-f][1,2,4]triazin-2-amine [1639];
(1r,4r)-4-((5-(imidazo[1,2-a]pyrimidin-6-yl)-4-(methoxy-d₃)pyrrolo[2,1-f][1,2,4]triazin-2-yl)amino)-1-methylcyclohexan-1-ol [1640];
(R)-cyclopropyl(3,3-difluoro-4-((5-(imidazo[1,2-a]pyrimidin-6-yl)-4-methoxypyrrolo[2,1-f][1,2,4]triazin-2-yl)amino)piperidin-1-yl)methanone [1641];
(R)—N-(3,3-difluoro-1-methylpiperidin-4-yl)-6-fluoro-5-(imidazo[1,2-a]pyrimidin-6-yl)-4-methoxypyrrolo[2,1-f][1,2,4]triazin-2-amine [1642];
(R)—N-(3,3-difluoro-1-(oxetan-3-yl)piperidin-4-yl)-6-fluoro-5-(imidazo[1,2-a]pyrimidin-6-yl)-4-methoxypyrrolo[2,1-f][1,2,4]triazin-2-amine [1643];
N-(4,4-difluorocyclohexyl)-5-(3-(difluoromethyl)imidazo[1,2-a]pyrimidin-6-yl)-4-methoxypyrrolo[2,1-f][1,2,4]triazin-2-amine [1644];
(1r,4r)-4-((5-(3-(difluoromethyl)imidazo[1,2-a]pyrimidin-6-yl)-4-methoxypyrrolo[2,1-f][1,2,4]triazin-2-yl)amino)-1-methylcyclohexan-1-ol [1645];
5-(3-(difluoromethyl)imidazo[1,2-a]pyrimidin-6-yl)-N-((3R,4S)-3-fluoro-1-(oxetan-3-yl)piperidin-4-yl)-4-methoxypyrrolo[2,1-f][1,2,4]triazin-2-amine [1646];
(R)—N-(3,3-difluoro-1-methylpiperidin-4-yl)-5-(3-(difluoromethyl)imidazo[1,2-a]pyrimidin-6-yl)-4-methoxypyrrolo[2,1-f][1,2,4]triazin-2-amine [1647];
(R)-1-(4-((5-(3-(difluoromethyl)imidazo[1,2-a]pyrimidin-6-yl)-4-methoxypyrrolo[2,1-f][1,2,4]triazin-2-yl)amino)-3,3-difluoropiperidin-1-yl)ethan-1-one [1648];
(R)—N-(3,3-difluoro-1-(oxetan-3-yl)piperidin-4-yl)-5-(3-(difluoromethyl)imidazo[1,2-a]pyrimidin-6-yl)-4-methoxypyrrolo[2,1-f][1,2,4]triazin-2-amine [1649];
3-((5-(3-(difluoromethyl)imidazo[1,2-a]pyrimidin-6-yl)-6-fluoro-4-methoxypyrrolo[2,1-f][1,2,4]triazin-2-yl)amino)-2,2-dimethylpropanenitrile [1650];
N-(4,4-difluorocyclohexyl)-5-(3-(difluoromethyl)imidazo[1,2-a]pyrimidin-6-yl)-6-fluoro-4-methoxypyrrolo[2,1-f][1,2,4]triazin-2-amine [1651];
(1s,4s)-4-((5-(3-(difluoromethyl)imidazo[1,2-a]pyrimidin-6-yl)-6-fluoro-4-methoxypyrrolo[2,1-f][1,2,4]triazin-2-yl)amino)-1-methylcyclohexan-1-ol [1652];
(1r,4r)-4-((5-(3-(difluoromethyl)imidazo[1,2-a]pyrimidin-6-yl)-6-fluoro-4-methoxypyrrolo[2,1-f][1,2,4]triazin-2-yl)amino)-1-methylcyclohexan-1-ol [1653];
(R)—N-(4,4-difluoro-1-methylpyrrolidin-3-yl)-5-(3-(difluoromethyl)imidazo[1,2-a]pyrimidin-6-yl)-6-fluoro-4-methoxypyrrolo[2,1-f][1,2,4]triazin-2-amine [1654];
(R)—N-(4,4-difluoro-1-(oxetan-3-yl)pyrrolidin-3-yl)-5-(3-(difluoromethyl)imidazo[1,2-a]pyrimidin-6-yl)-6-fluoro-4-methoxypyrrolo[2,1-f][1,2,4]triazin-2-amine [1655];
(R)-1-(4-((5-(3-(difluoromethyl)imidazo[1,2-a]pyrimidin-6-yl)-6-fluoro-4-methoxypyrrolo[2,1-f][1,2,4]triazin-2-yl)amino)-3,3-difluoropiperidin-1-yl)ethan-1-one [1656];
5-(3-(difluoromethyl)imidazo[1,2-a]pyrimidin-6-yl)-6-fluoro-4-methoxy-N-(1-(oxetan-3-yl)piperidin-4-yl)pyrrolo[2,1-f][1,2,4]triazin-2-amine [1657];
5-(3-(difluoromethyl)imidazo[1,2-a]pyrimidin-6-yl)-6-fluoro-N-((3S,4R)-3-fluoro-1-(oxetan-3-yl)piperidin-4-yl)-4-methoxypyrrolo[2,1-f][1,2,4]triazin-2-amine [1658];
5-(3-(difluoromethyl)imidazo[1,2-a]pyrimidin-6-yl)-6-fluoro-N-((3R,4R)-3-fluoro-1-(oxetan-3-yl)piperidin-4-yl)-4-methoxypyrrolo[2,1-f][1,2,4]triazin-2-amine [1659];
5-(3-(difluoromethyl)imidazo[1,2-a]pyrimidin-6-yl)-6-fluoro-N-((3S,4S)-3-fluoro-1-(oxetan-3-yl)piperidin-4-yl)-4-methoxypyrrolo[2,1-f][1,2,4]triazin-2-amine [1660];
5-(3-(difluoromethyl)imidazo[1,2-a]pyrimidin-6-yl)-6-fluoro-N-((3R,4S)-3-fluoro-1-(oxetan-3-yl)piperidin-4-yl)-4-methoxypyrrolo[2,1-f][1,2,4]triazin-2-amine [1661];
(R)—N-(3,3-difluoro-1-methylpiperidin-4-yl)-5-(3-(difluoromethyl)imidazo[1,2-a]pyrimidin-6-yl)-6-fluoro-4-methoxypyrrolo[2,1-f][1,2,4]triazin-2-amine [1662];
(R)—N-(3,3-difluoro-1-(methyl-d₃)piperidin-4-yl)-5-(3-(difluoromethyl)imidazo[1,2-a]pyrimidin-6-yl)-6-fluoro-4-methoxypyrrolo[2,1-f][1,2,4]triazin-2-amine [1663];
(R)—N-(3,3-difluoro-1-(oxetan-3-yl)piperidin-4-yl)-5-(3-(difluoromethyl)imidazo[1,2-a]pyrimidin-6-yl)-6-fluoro-4-methoxypyrrolo[2,1-f][1,2,4]triazin-2-amine [1664];
5-(3-(difluoromethyl)imidazo[1,2-a]pyrimidin-6-yl)-6-fluoro-4-methoxy-N-(2-oxaspiro[3.5]nonan-7-yl)pyrrolo[2,1-f][1,2,4]triazin-2-amine [1665];
6-(2-((4,4-difluorocyclohexyl)amino)-4-methoxypyrrolo[2,1-f][1,2,4]triazin-5-yl)-N-methylimidazo[1,2-a]pyrimidine-3-carboxamide [1666];
6-(2-((4,4-dimethylcyclohexyl)amino)-4-methoxypyrrolo[2,1-f][1,2,4]triazin-5-yl)-N-methylimidazo[1,2-a]pyrimidine-3-carboxamide [1667];
6-(4-methoxy-2-(spiro[2.5]octan-6-ylamino)pyrrolo[2,1-f][1,2,4]triazin-5-yl)-N-methylimidazo[1,2-a]pyrimidine-3-carboxamide [1668];
6-(2-(((1r,4r)-4-hydroxy-4-methylcyclohexyl)amino)-4-methoxypyrrolo[2,1-f][1,2,4]triazin-5-yl)-N-methylimidazo[1,2-a]pyrimidine-3-carboxamide [1669];
(R)-6-(2-((3,3-difluoro-1-methylpiperidin-4-yl)amino)-4-methoxypyrrolo[2,1-f][1,2,4]triazin-5-yl)-N-methylimidazo[1,2-a]pyrimidine-3-carboxamide [1670];
(R)-6-(2-((3,3-difluoro-1-(oxetan-3-yl)piperidin-4-yl)amino)-4-methoxypyrrolo[2,1-f][1,2,4]triazin-5-yl)-N-methylimidazo[1,2-a]pyrimidine-3-carboxamide [1671];
6-(2-((2-cyano-2-methylpropyl)amino)-6-fluoro-4-methoxypyrrolo[2,1-f][1,2,4]triazin-5-yl)-N-methylimidazo[1,2-a]pyrimidine-3-carboxamide [1672];
6-(2-((4,4-dimethylcyclohexyl)amino)-6-fluoro-4-methoxypyrrolo[2,1-f][1,2,4]triazin-5-yl)-N-methylimidazo[1,2-a]pyrimidine-3-carboxamide [1673];
6-(6-fluoro-4-methoxy-2-(spiro[2.5]octan-6-ylamino)pyrrolo[2,1-f][1,2,4]triazin-5-yl)-N-methylimidazo[1,2-a]pyrimidine-3-carboxamide [1674];
6-(2-((4,4-difluorocyclohexyl)amino)-6-fluoro-4-methoxypyrrolo[2,1-f][1,2,4]triazin-5-yl)-N-methylimidazo[1,2-a]pyrimidine-3-carboxamide [1675];
6-(6-fluoro-2-(((1s,4s)-4-hydroxy-4-methylcyclohexyl)amino)-4-methoxypyrrolo[2,1-f][1,2,4]triazin-5-yl)-N-methylimidazo[1,2-a]pyrimidine-3-carboxamide [1676];
6-(6-fluoro-2-(((1r,4r)-4-hydroxy-4-methylcyclohexyl)amino)-4-methoxypyrrolo[2,1-f][1,2,4]triazin-5-yl)-N-methylimidazo[1,2-a]pyrimidine-3-carboxamide [1677];
(R)-6-(2-((4,4-difluoro-1-methylpyrrolidin-3-yl)amino)-6-fluoro-4-methoxypyrrolo[2,1-f][1,2,4]triazin-5-yl)-N-methylimidazo[1,2-a]pyrimidine-3-carboxamide [1678];
(R)-6-(2-((4,4-difluoro-1-(oxetan-3-yl)pyrrolidin-3-yl)amino)-6-fluoro-4-methoxypyrrolo[2,1-f][1,2,4]triazin-5-yl)-N-methylimidazo[1,2-a]pyrimidine-3-carboxamide [1679];
6-(6-fluoro-4-methoxy-2-((1-(oxetan-3-yl)piperidin-4-yl)amino)pyrrolo[2,1-f][1,2,4]triazin-5-yl)-N-methylimidazo[1,2-a]pyrimidine-3-carboxamide [1680];
6-(6-fluoro-2-(((3S,4R)-3-fluoro-1-(oxetan-3-yl)piperidin-4-yl)amino)-4-methoxypyrrolo[2,1-f][1,2,4]triazin-5-yl)-N-methylimidazo[1,2-a]pyrimidine-3-carboxamide [1681];
6-(6-fluoro-2-(((3R,4R)-3-fluoro-1-(oxetan-3-yl)piperidin-4-yl)amino)-4-methoxypyrrolo[2,1-f][1,2,4]triazin-5-yl)-N-methylimidazo[1,2-a]pyrimidine-3-carboxamide [1682];
6-(6-fluoro-2-(((3S,4S)-3-fluoro-1-(oxetan-3-yl)piperidin-4-yl)amino)-4-methoxypyrrolo[2,1-f][1,2,4]triazin-5-yl)-N-methylimidazo[1,2-a]pyrimidine-3-carboxamide [1683];
6-(6-fluoro-2-(((3R,4S)-3-fluoro-1-(oxetan-3-yl)piperidin-4-yl)amino)-4-methoxypyrrolo[2,1-f][1,2,4]triazin-5-yl)-N-methylimidazo[1,2-a]pyrimidine-3-carboxamide [1684];
(R)-6-(2-((3,3-difluoro-1-methylpiperidin-4-yl)amino)-6-fluoro-4-methoxypyrrolo[2,1-f][1,2,4]triazin-5-yl)-N-methylimidazo[1,2-a]pyrimidine-3-carboxamide [1685];
(R)-6-(2-((3,3-difluoro-1-(methyl-d₃)piperidin-4-yl)amino)-6-fluoro-4-methoxypyrrolo[2,1-f][1,2,4]triazin-5-yl)-N-methylimidazo[1,2-a]pyrimidine-3-carboxamide [1686];
(R)-6-(2-((3,3-difluoro-1-(oxetan-3-yl)piperidin-4-yl)amino)-6-fluoro-4-methoxypyrrolo[2,1-f][1,2,4]triazin-5-yl)-N-methylimidazo[1,2-a]pyrimidine-3-carboxamide [1687];
6-(2-((2-oxaspiro[3.5]nonan-7-yl)amino)-6-fluoro-4-methoxypyrrolo[2,1-f][1,2,4]triazin-5-yl)-N-methylimidazo[1,2-a]pyrimidine-3-carboxamide [1688];
(S)-1-((3R,4S)-3-fluoro-4-((4-methoxy-5-(quinolin-6-yl)pyrrolo[2,1-f][1,2,4]triazin-2-yl)amino)piperidin-1-yl)propan-2-ol [1689];
(R)-1-((3R,4S)-3-fluoro-4-((4-methoxy-5-(quinolin-6-yl)pyrrolo[2,1-f][1,2,4]triazin-2-yl)amino)piperidin-1-yl)propan-2-ol [1690];
(R)-2-((3R,4S)-3-fluoro-4-((4-methoxy-5-(quinolin-6-yl)pyrrolo[2,1-f][1,2,4]triazin-2-yl)amino)piperidin-1-yl)propan-1-ol [1691];
(S)-2-((3R,4S)-3-fluoro-4-((4-methoxy-5-(quinolin-6-yl)pyrrolo[2,1-f][1,2,4]triazin-2-yl)amino)piperidin-1-yl)propan-1-ol [1692];
(R)—N-(3,3-difluoropiperidin-4-yl)-5-(8-fluoroquinolin-6-yl)-4-methoxypyrrolo[2,1-f][1,2,4]triazin-2-amine [1693];
(R)—N-(3,3-difluoro-1-methylpiperidin-4-yl)-5-(8-fluoroquinolin-6-yl)-4-methoxypyrrolo[2,1-f][1,2,4]triazin-2-amine [1694];
(R)-1-(3,3-difluoro-4-((5-(8-fluoroquinolin-6-yl)-4-methoxypyrrolo[2,1-f][1,2,4]triazin-2-yl)amino)piperidin-1-yl)ethan-1-one [1695];
(R)—N-(3,3-difluoro-1-(oxetan-3-yl)piperidin-4-yl)-5-(8-fluoroquinolin-6-yl)-4-methoxypyrrolo[2,1-f][1,2,4]triazin-2-amine [1696];
1-((3R,4S)-3-((5-(1-(2,2-difluoroethyl)-2-methyl-1H-benzo[d]imidazol-6-yl)-6-fluoro-4-methoxypyrrolo[2,1-f][1,2,4]triazin-2-yl)amino)-4-fluoropyrrolidin-1-yl)ethan-1-one [1697];
5-(1-(2,2-difluoroethyl)-2-methyl-1H-benzo[d]imidazol-6-yl)-6-fluoro-N-((3R,4S)-4-fluoro-1-(oxetan-3-yl)pyrrolidin-3-yl)-4-methoxypyrrolo[2,1-f][1,2,4]triazin-2-amine [1698];

34. A pharmaceutical composition comprising a therapeutically effective amount of a compound of claim 1, or a pharmaceutically acceptable salt thereof, and a pharmaceutically acceptable excipient.

35. A method of treating a disorder or disease in a patient, wherein the disorder or disease is selected from the group consisting of: a neurological disorder, diabetes, and cancer, the method comprising administering to the patient a therapeutically effective amount of a compound of claim 1 or a pharmaceutically acceptable salt, or the pharmaceutical composition of claim 34.

36. The method of claim 35, wherein the disorder or disease is cancer.

37. The method of claim 35, wherein the disorder or disease is diabetes.

38. The method of claim 35, wherein the disorder or disease is a neurological disorder.

39. The method of claim 36, wherein the cancer is selected from the group consisting of: brain tumors, glioblastoma, ovarian, breast, head and neck squamous cell carcinoma, hepatocellular carcinoma, pancreatic cancer, acute lymphoblastic leukemia, acute megakaryoblastic leukemia, and chronic myeloid leukemia.

40. The method of claim 38, wherein the disorder or disease is a neurological disorder, wherein the neurological disorder is selected from the group consisting of: Alzheimer's disease, amyotrophic lateral sclerosis, CDKL5 deficiency disorder, Down syndrome, frontotemporal dementia with parkinsonism-17 (FTDP-17), Lewy body dementia, Parkinson's disease, Pick's disease, and additional diseases with pronounced neurodegeneration such as autism, dementia, epilepsy, Huntington's disease, multiple sclerosis; diseases and disorders associated with acquired brain injury such as chronic traumatic encephalopathy, traumatic brain injury, tumor, stroke, Pick disease, progressive supranuclear palsy, corticobasal degeneration, argyrophilic grain disease, globular glial tauopathies, primary age-related tauopathy, neurofibrillary tangle dementia, chronic traumatic encephalopathy (CTE), frontotemporal lobar degeneration with tau inclusions (FTLD-tau), and aging-related tau astrogliopathy.

41. The method of claim 40, wherein the disorder or disease is Alzheimer's disease.

42. The method of claim 35, wherein the patient is a human.
